(19) United States
(12) Patent Application Publication
(10) Pub. No.: US 2024/0150848 A1
(43) Pub. Date: May 9, 2024

(54) METHODS AND SYSTEMS FOR DIAGNOSIS, CLASSIFICATION, AND TREATMENT OF SMALL CELL LUNG CANCER AND OTHER HIGH-GRADE NEUROENDOCRINE CARCINOMAS

(71) Applicant: BOARD OF REGENTS, THE UNIVERSITY OF TEXAS SYSTEM, Austin, TX (US)

(72) Inventors: John V. HEYMACH, Houston, TX (US); Simon HEEKE, Houston, TX (US); Lauren A. Byers, Houston, TX (US); Carl M. GAY, Houston (CO)

(73) Assignee: BOARD OF REGENTS, THE UNIVERSITY OF TEXAS SYSTEM, Austin, TX (US)

(21) Appl. No.: 18/548,865

(22) PCT Filed: Mar. 3, 2022
(86) PCT No.: PCT/US2022/018797

§ 371 Date: Sep. 1, 2023

Related U.S. Application Data
(60) Provisional application No. 63/156,005, filed on Mar. 3, 2021.
(60) Provisional application No. 63/276,475, filed on Nov. 5, 2021.

(51) Int. Cl. C12Q 1/6886

(52) U.S. Cl. CPC C12Q 1/6886; C12Q 2600/106; C12Q 2600/154

(57) ABSTRACT

Aspects of the disclosure are directed to methods and systems for diagnosis, classification, and treatment of small cell lung cancer and high grade neuroendocrine carcinomas. Certain aspects comprise detection and analysis of tumor DNA methylation. Included in the disclosure are methods for identifying a subject as having a particular small cell lung cancer subtype based on DNA N methylation analysis, as well as methods and compositions for treatment of a subject based on subtype classification.

### CROSS-REFERENCE TO RELATED APPLICATIONS

The present application claims the priority benefit of U.S. provisional application No. 63/156,005, filed Mar. 3, 2021, and U.S. provisional application No. 63/276,475, filed Nov. 5, 2021, which applications are incorporated herein by reference in their entirety.

This invention was made with government support under grant numbers CA213273 and CA256780 awarded by the National Institutes of Health. The government has certain rights in the invention.

### BACKGROUND

### I. Field of the Invention

Aspects of this invention relate to at least the fields of cancer biology and medicine.

### II. Background

For patients with small cell lung cancer (SCLC) or other high-grade neuroendocrine carcinomas (HGNEC), there are currently no validated biomarkers in routine use for classifying the disease into therapeutically relevant subgroups and predicting response to different therapies; nor are there blood-based biomarkers for diagnosing SCLC or HGNEC and distinguishing it from other types of cancers. Unlike non-small cell lung cancer (NSCLC), for which there are well established genomic markers (e.g. EGFR mutation) and proteomic markers (e.g. PD-L1) for selecting treatments, there are currently no similar markers in SCLC or HGNEC. One challenge in diagnosis, treatment, and analysis of SCLC and HGNEC is that there is usually limited tumor tissue available for analysis. The identification of biomarkers that can help classify SCLC and HGNEC, predict response to therapy, and distinguish it from other tumor types therefore represents a fundamental unmet need for SCLC, particularly if such analysis could be performed using minimal tissue or other sources more readily available such as blood.

### SUMMARY

The present disclosure fulfills certain needs in the fields of cancer biology and medicine by providing methods and compositions for diagnosis, classification, and treatment of SCLC and SCLC subtypes. Embodiments are directed to compositions and methods for identifying a subject as having SCLC-A, SCLC-N, SCLC-P. or SCLC-I based on analysis of methylation sites from tumor DNA from the subject. Also disclosed are methods for treatment of a subject having SCLC-A, SCLC-N, SCLC-P, or SCLC-I.

Embodiments of the present disclosure include methods for treating a subject for SCLC, methods for diagnosing a subject with SCLC, methods for identifying a subject with cancer as having SCLC, methods for classifying SCLC of a subject, methods for identifying a subject as having SCLC-A, methods for identifying a subject as having SCLC-N, methods for identifying a subject as having SCLC-P, methods for identifying a subject as having SCLC-I, methods for analysis of tumor methylation, methods for prognosing a subject with SCLC, methods for selecting a treatment for a subject with SCLC, and kits for nucleic acid analysis. Methods of the present disclosure can include at least 1, 2, 3, 4, 5, or more of the following steps: obtaining a biological sample from a subject, obtaining tumor DNA from a subject, classifying a subject as having SCLC-A, classifying a subject as having SCLC-N, classifying a subject as having SCLC-P, classifying a subject as having SCLC-I, determining a methylation status of a methylation site, analyzing tumor DNA from a subject, diagnosing a subject for SCLC, treating a subject for SCLC, administering a BCL2 inhibitor to a subject, administering a DLL3-targeted therapy to a subject, administering an AURK inhibitor to a subject, administering a platinum-based chemotherapeutic agent to a subject, administering a PARP inhibitor to a subject, administering an anti-metabolite to a subject, administering a nucleoside analog to a subject, administering an immunotherapy to a subject, administering a chemotherapy to a subject, and administering a radiotherapy to a subject. One or more of the following steps may be included from embodiments of the disclosure.

Embodiments of the disclosure are directed to a method of treating a subject for small cell lung cancer (SCLC), the method comprising administering a BCL2 inhibitor or a DLL3-targeted therapy to a subject determined, from analysis of tumor DNA from the subject, to have differential methylation at two or more methylation sites selected from the methylation sites of Table 2, Table 7, Table 15, Table 20, and Table 27 compared to a reference or control sample. In some embodiments, the two or more methylation sites are at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range or value derivable therein) selected from the methyation sites of Table 2, Table 7, Table 15, Table 20, and Table 27, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 2, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 7, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 15, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 20, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 27, or more. In some embodiments, the method comprises administering the BCL2 inhibitor to the subject. In some embodiments, the BCL2 inhibitor is ABT-737 or navitoclax. In some embodiments, the method comprises administering the DLL3-targeted therapy to the subject. In some embodiments, the DLL3-targeted therapeutic comprises an anti-DLL3 antibody or fragment thereof. In some embodiments, the anti-DLL3 antibody or fragment thereof is rovalpituzumab. In some embodiments, the DLL3-targeted therapeutic is an antibody-drug conjugate. In some embodiments, the antibody-drug conjugate is rovalpituzumab tesirine. In some embodiments, the DLL3-targeted therapeutic is a DLL3-targeted cellular therapy. A DLL3-targeted cellular therapy can include any cell-based therapy for targeting DLL3. In some embodiments, the DLL3-targeted cellular therapy is a DLL3-targeted chimeric antigen receptor (CAR) T-cell. In some embodiments, the DLL3-targeted cellular therapy is a DLL3-targeted CAR NK cell. In some embodiments, the subject was determined to have SCLC-A based on the analysis of the tumor DNA. It is specifically contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range derivable therein), or more, selected from the methyation sites of Table 2, Table 7, Table 15, Table 20, and Table 27, may be excluded in embodiments described herein.

Embodiments of the disclosure are directed to a method of treating a subject for SCLC, the method comprising administering an AURK inhibitor to a subject determined, from analysis of tumor DNA from the subject, to have differential methylation at two or more methylation sites selected from the methylation sites of Table 3, Table 8, Table 16, Table 21, and Table 28 compared to a reference or control sample. In some embodiments, the two or more methylation sites are at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range or value derivable therein) selected from the methyation sites of Table 3, Table 8, Table 16, Table 21, and Table 28. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 3, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 8, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 16, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 21, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 28, or more. In some embodiments, the AURK inhibitor is CYC-116, alisertib, or AS-703569. In some embodiments, the subject was determined to have SCLC-N based on the analysis of the tumor DNA. It is specifically contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range derivable therein), or more, selected from the methyation sites of Table 3, Table 8, Table 16, Table 21, and Table 28, may be excluded in embodiments described herein.

Embodiments of the disclosure are directed to a method of treating a subject for SCLC, the method comprising administering a platinum-based chemotherapeutic agent, a PARP inhibitor, an anti-metabolite, or a nucleoside analog to a subject determined, from analysis of tumor DNA from the subject, to have differential methylation at two or more methylation sites selected from the methylation sites of Table 4, Table 9, Table 17, Table 22, and Table 29 compared to a reference or control sample. In some embodiments, the two or more methylation sites are at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range or value derivable therein) selected from the methyation sites of Table 4, Table 9, Table 17, Table 22, and Table 29, or more. In some embodiments, the two or more methylation sites are at least or at most 2, 3, 4, 5, 6, 7, 8, 9, 10, 15, or 20 (or any range derivable therein) of the methylation sites of Table 4, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 9, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 17, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 22, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 29, or more. In some embodiments, the method comprises administering to the subject the platinum-containing chemotherapeutic agent. In some embodiments, the platinum-containing chemotherapeutic agent is cisplatin, carboplatin, oxaliplatin, nedaplatin, picoplatin, or satraplatin. In some embodiments, the method comprises administering to the subject the PARP inhibitor. In some embodiments, the PARP inhibitor is talazoparib, olaparib, niraparib, AZD-2461, or rucaparib. In some embodiments, the method comprises administering to the subject the anti-metabolite. In some embodiments, the anti-metabolite is pemetrexed, methotrexate, or pralatrexate. In some embodiments, the method comprises administering to the subject the nucleoside analog. In some embodiments, the nucleoside analog is floxuridine, cytarabine, clofarabine, or fludarabine. In some embodiments, the subject was determined to have SCLC-P based on the analysis of the tumor DNA. It is specifically contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range derivable therein), or more, selected from the methyation sites of Table 4, Table 9, Table 17, Table 22, and Table 29, may be excluded in embodiments described herein.

Embodiments of the disclosure are directed to a method of treating a subject for SCLC, the method comprising administering an immunotherapy to a subject determined, from analysis of tumor DNA from the subject, to have differential methylation at two or more methylation sites selected from the methylation sites of Table 5. Table 10, Table 18, Table 23, and Table 30 compared to a reference or control sample. In some embodiments, the two or more methylation sites are at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range or value derivable therein) selected from the methyation sites of Table 5. Table 10, Table 18, Table 23, and Table 30, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 5, or more. In some embodiments, the two or more methylation sites are at least or at most 2, 3, 4, 5, 6, 7, 8, 9, 10, 11, 12, 13, 14, 15, 16, 17, 18, 19, or 20 (or any range derivable therein) of the methylation sites of Table 10, or more. In some embodiments, the two or more methylation sites are at least 2, 3, 4, 5, 6, 7, 8, 9, 10, 11, 12, 13, 14, 15, 16, 17, 18, 19, or 20 (or any range derivable therein) of the methylation sites of Table 18, or more. In some embodiments, the two or more methylation sites are at least 2, 3, 4, 5, 6, 7, 8, 9, 10, 11, 12, 13, 14, 15, 16, 17, 18, 19, or 20 (or any range derivable therein) of the methylation sites of Table 23, or more. In some embodiments, the two or more methylation sites are at least 2, 3, 4, 5, 6, 7, 8, 9, 10, 11, 12, 13, 14, 15, 16, 17, 18, 19, or 20 (or any range derivable therein) of the methylation sites of Table 30, or more. In some embodiments, the immunotherapy is an immune checkpoint inhibitor therapy. In some embodiments, the subject was determined to have SCLC-I based on the analysis of the tumor DNA. It is specifically contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 methylation sites (or any range derivable therein), or more, selected from the methyation sites of Table 5, Table 10, Table 18, Table 23, and Table 30, may be excluded in embodiments described herein.

In some embodiments, the method further comprises administering to the subject an additional cancer therapy. In some embodiments, the additional cancer therapy comprises chemotherapy, radiotherapy, immunotherapy, or a combination thereof. In some embodiments, the subject was previously treated for SCLC. In some embodiments, the subject was resistant to the previous treatment.

In some embodiments, the subject was further determined, from the analysis of the tumor DNA from the subject, to have differential methylation of one or more methylation sites of Table 13 compared to the reference or control sample. In some embodiments, the reference or control sample is a DNA sample obtained from healthy cells from the subject. In some embodiments, the reference or control sample is a DNA sample obtained from a cell-free sample (e.g., plasma, serum) from a reference subject. In some embodiments, the reference or control sample is a DNA sample obtained from healthy cells from a reference subject. In some embodiments, the reference or control sample is a DNA sample obtained from a cell-free sample (e.g., plasma, serum) from a reference subject.

Embodiments of the disclosure are directed to a method for classifying a subject having SCLC, the method comprising (a) determining, from DNA from the subject, a methylation status of one or more methylation sites selected from the methylation sites of Tables 1-10, 15-18, 20-23, and 27-30; and (b) classifying the subject as having SCLC-A, SCLC-N, SCLC-P, or SCLC-I based on the methylation status of the one or more methylation sites. In some embodiments, the one or more methylation sites are at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 150, or 200 of the methylation sites (or more) selected from the methylation sites of Tables 1-10, 15-18, 20-23, and 27-30.

In some embodiments, (b) comprises classifying the subject as having SCLC-A. In some embodiments, the one or more methylation sites are at least 2, 3, 4, 5, 6, 7, 8, 9, 10, 11, 12, 13, 14, 15, 16, 17, 18, 19, or 20 of the methylation sites selected from the methylation sites of Table 2, Table 7, Table 15, Table 20, and Table 27 or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 2, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 7, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 15, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 20, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 27, or more. In some embodiments, the method further comprises administering to the subject a therapeutically effective amount of a BCL2 inhibitor. In some embodiments, the BCL2 inhibitor is ABT-737 or navitoclax. In some embodiments, the method further comprises administering to the subject a therapeutically effective amount of a DLL3-targeted therapeutic. In some embodiments, the DLL3-targeted therapeutic comprises an anti-DLL3 antibody or fragment thereof. In some embodiments, the anti-DLL3 antibody or fragment thereof is rovalpituzumab. In some embodiments, the DLL3-targeted therapeutic is an antibody-drug conjugate. In some embodiments, the antibody-drug conjugate is rovalpituzumab tesirine.

In some embodiments, (b) comprises classifying the subject as having SCLC-N. In some embodiments, the one or more methylation sites are at least 2, 3, 4, 5, 6, 7, 8, 9, 10, 11, 12, 13, 14, 15, 16, 17, 18, 19, or 20 of the methylation sites selected from the methylation sites of Table 3, Table 8, Table 16, Table 21, and Table 28, or more. In some embodiments, the one or more methylation sites are at least 2, 3, 4, 5, 6, 7, 8, 9, 10, 15, or 20 of the methylation sites of Table 3, or more. In some embodiments, the one or more methylation sites are at least 2, 3, 4, 5, 6, 7, 8, 9, 10, 15, or 20 of the methylation sites of Table 8, or more. In some embodiments, the one or more methylation sites are at least 2, 3, 4, 5, 6, 7, 8, 9, 10, 15, or 20 of the methylation sites of Table 16, or more. In some embodiments, the one or more methylation sites are at least 2, 3, 4, 5, 6, 7, 8, 9, 10, 15, or 20 of the methylation sites of Table 21, or more. In some embodiments, the one or more methylation sites are at least 2, 3, 4, 5, 6, 7, 8, 9, 10, 15, or 20 of the methylation sites of Table 28, or more. In some embodiments, the method further comprises administering to the subject a therapeutically effective amount of an AURK inhibitor. In some embodiments, the AURK inhibitor is CYC-116, alisertib, or AS-703569.

In some embodiments, (b) comprises classifying the subject as having SCLC-P. In some embodiments, the one or more methylation sites are at least 2, 3, 4, 5, 6, 7, 8, 9, 10, 11, 12, 13, 14, 15, 16, 17, 18, 19, or 20 of the methylation sites selected from the methylation sites of Table 4, Table 9, Table 17, Table 22, and Table 29, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 4, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 9, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 17, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 22, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 29, or more. In some embodiments, the method further comprises administering to the subject a therapeutically effective amount of a platinum-containing chemotherapeutic agent. In some embodiments, the platinum-containing chemotherapeutic agent is cisplatin, carboplatin, oxaliplatin, nedaplatin, picoplatin, or satraplatin. In some embodiments, the method further comprises administering to the subject a therapeutically effective amount of a PARP inhibitor. In some embodiments, the PARP inhibitor is talazoparib, olaparib, niraparib, AZD-2461, or rucaparib. In some embodiments, the method further comprises administering to the subject a therapeutically effective amount of an anti-metabolite. In some embodiments, the anti-metabolite is pemetrexed, methotrexate, or pralatrexate. In some embodiments, the method further comprises administering to the subject a therapeutically effective amount of a nucleoside analog. In some embodiments, the nucleoside analog is floxuridine, cytarabine, clofarabine, or fludarabine.

In some embodiments, (b) comprises classifying the subject as having SCLC-I. In some embodiments, the one or more methylation sites are at least 2, 3, 4, 5, 6, 7, 8, 9, 10, 11, 12, 13, 14, 15, 16, 17, 18, 19, or 20 of the methylation sites selected from the methyation sites of Table 5, Table 10, Table 18, Table 23, and Table 30, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 5, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 10, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 18, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 23, or more. In some embodiments, the one or more methylation sites are at least 2, 3, 4, 5, 6, 7, 8, 9, 10, 11, 12, 13, 14, 15, 16, 17, 18, 19, or 20 of the methylation sites of Table 30, or more. In some embodiments, the method further comprises administering to the subject a therapeutically effective amount of an immunotherapy. In some embodiments, the immunotherapy is a checkpoint blockade therapy.

In some embodiments, DNA from the subject is obtained from blood or plasma from the subject. In some embodiments, the DNA is circulating tumor DNA (ctDNA). In some embodiments, the DNA is obtained from cancer tissue from the subject.

In some embodiments, the method further comprises determining, from the DNA from the subject, a methylation status of one or more methylation sites of Table 13. In some embodiments, the one or more methylation sites comprise, comprise at least, or comprise at most 1, 2, 3, 4, 5, 6, 7, 8, 9, 10, 11, 12, 13, 14, 15, 16, 17, 18, 19, 20, 21, 22, 23, 24, 25, 26, or 27 of the methylation sites of Table 13.

Embodiments are directed to a method of identifying a subject with cancer as having SCLC, the method comprising (a) determining, from DNA from the subject, a methylation status of one or more methylation sites of Table 13; and (b) identifying the subject as having SCLC based on the methylation status of the two or more methylation sites. In some embodiments, the one or more methylation sites are, are at least, or are at most 1, 2, 3, 4, 5, 6, 7, 8, 9, 10, 11, 12, 13, 14, 15, 16, 17, 18, 19, 20, 21, 22, 23, 24, 25, 26, or 27 of the methylation sites of Table 13.

Embodiments are directed to a method for treating a subject for SCLC comprising administering an SCLC therapy to a subject determined, from analysis of tumor DNA from the subject, to have differential methylation at one or more methylation sites of Table 13 compared to a reference or control sample. In some embodiments, the SCLC therapy comprises chemotherapy, immunotherapy, radiotherapy, or a combination thereof. In some embodiments, the SCLC therapy comprises a platinum-containing chemotherapeutic agent. In some embodiments, the platinum-containing chemotherapeutic agent is cisplatin, carboplatin, oxaliplatin, nedaplatin, picoplatin, or satraplatin.

Further embodiments include use of an SCLC therapy for treatment of a subject having differential methylation at one or more methylation sites of Table 13 compared to a reference or control sample. In some embodiments, the one or more methylation sites are, are at least, or are at most 1, 2, 3, 4, 5, 6, 7, 8, 9, 10, 11, 12, 13, 14, 15, 16, 17, 18, 19, 20, 21, 22, 23, 24, 25, 26, or 27 of the methylation sites of Table 13. In some embodiments, the SCLC therapy comprises a platinum-containing chemotherapeutic agent. In some embodiments, the platinum-containing chemotherapeutic agent is cisplatin, carboplatin, oxaliplatin, nedaplatin, picoplatin, or satraplatin.

Additional aspects of the disclosure include methods for treating a subject for SCLC comprising administering a therapeutically effective amount of HG-5-88-01, ZG-10, BI-2536, Dinaciclib, GW843682X, OTX015, Sinularin, Sunitinib, ULK1 4989, GSK591, or JAK1 8709 to a subject determined, from analysis of tumor DNA from the subject, to have differential methylation at chr4:152172102 (cg06883206), chr1:155177783 (cg02516288), chr17:46804309 (cg16557178), chr14:56604516 (cg16770832), chr17:46804309 (cg16557178), chr7:26192756 (cg14644871), chr9:130659142 (cg03083695), chr1:15272238 (cg11648522), chr14:63872285 (cg02929982), chr8:42132866 (cg05570682), or chr19:47156401 (cg22981158), respectively, compared to a reference or control sample.

Where the present application refers to methods performed on a subject determined, from analysis of tumor DNA from the subject, to have differential methylation, embodiments are also directed to those same methods performed on a subject having that differential methylation. In these embodiments the differential methylation may be determined by analyzing tumor DNA from the subject.

Further embodiments include a method of diagnosing small cell lung cancer (SCLC) in a subject, comprising analyzing tumor DNA from the subject, wherein differential methylation compared to a reference or control sample at one or more methylation sites selected from the methylation sites listed in Tables 2, 3, 4, 5, 7, 8, 9, 10, 15, 16, 17, 18, 20, 21, 22, 23, 27, 28, 29, and/or 30 indicates that the subject has SCLC.

Further embodiments include the use of differential methylation compared to a reference or control sample at one or more methylation sites selected from the methylation sites listed in Tables 2, 3, 4, 5, 7, 8, 9, 10, 15, 16, 17, 18, 20, 21, 22, 23, 27, 28, 29, and/or 30 in a method of diagnosing small cell lung cancer (SCLC) in a subject. In certain embodiments, the use comprises analyzing tumor DNA from the subject, wherein differential methylation compared to a reference or control sample at one or more methylation sites selected from the methylation sites listed in Tables 2, 3, 4, 5, 7, 8, 9, 10, 15, 16, 17, 18, 20, 21, 22, 23, 27, 28, 29, and/or 30 indicates that the subject has SCLC.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A. B, and/or C includes: A alone, B alone, C alone, a combination of A and B, a combination of A and C, a combination of B and C. or a combination of A. B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject,” and “patient” are used interchangeably and can refer to a human or non-human.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary, Detailed Description. Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and 1B shows clustering of methylation sites and SCLC-subtypes in the GDSC (A) and the NCI (B) dataset. The top 1000 methylation sites for each subtype defined by AUROC analysis were selected for the analysis.

FIGS. 2A and 2B demonstrate the diagnostic performance of the models trained to predict SCLC subtypes using the top 15,000 methylation sites for each subtype. Models were trained on the NCI dataset and used to predict SCLC subtypes in the GDSC dataset in the whole dataset (solid line) and the cell lines that were unique to the GDSC dataset (dotted line). Only models created to predict the SCLC-A (A) and the SCLC-N (B) subtypes are shown. Two different models, net-elastic logistic regression (GLM) and random forest (RF), were used.

FIG. 3 demonstrates the use of combinations of two markers to predict the SCLC-A subset with models trained using the GDSC dataset. 1378 combinations were tested and results are ordered by the highest AUROC (ROC). Sensitivity (Sens) and specificity (Spec) is highlighted with the 95% CI in blue bars.

FIG. 4 demonstrates the use of combinations of two markers to predict the SCLC-N subset with models trained using the GDSC dataset. 2211 combinations were tested and results are ordered by the highest AUROC (ROC). Sensitivity (Sens) and specificity (Spec) is highlighted with the 95% CI in blue bars.

FIG. 5 shows the prediction of different SCLC subtypes using the GSE56044 dataset consisting of 124 lung cancers of different histology. AC: Adenocarcinoma. AdenoSq: Adenosquamous carcinoma. LC: Large cell carcinoma. LCNEC: Large cell neuroendocrine carcinoma. SCLC: Small cell lung cancer. SqCC: Squamous cell carcinoma.

FIG. 6 shows the prediction of subtypes using the GSE56044 dataset using large models (including 15,000 Methylation markers) and reduced panels consisting of two markers (upper heatmap) and three markers (middle heatmap) using models to specifically predict the SCLC-A subtype. AC: Adenocarcinoma. AdenoSq: Adenosquamous carcinoma. LC: Large cell carcinoma. LCNEC: Large cell neuroendocrine carcinoma. SCLC: Small cell lung cancer. SqCC: Squamous cell carcinoma.

FIG. 7 shows the prediction of subtypes using the GSE56044 dataset using large models (including 15,000 Methylation markers) and reduced panels consisting of two markers (upper heatmap) and three markers (middle heatmap) using models to specifically predict the SCLC-N subtype. AC: Adenocarcinoma. AdenoSq: Adenosquamous carcinoma. LC: Large cell carcinoma. LCNEC: Large cell neuroendocrine carcinoma. SCLC: Small cell lung cancer. SqCC: Squamous cell carcinoma.

FIG. 8 shows the prediction of subtypes using the GSE56044 dataset using large models (including 15,000 Methylation markers) and reduced panels consisting of two markers (upper heatmap) and three markers (middle heatmap) using models to specifically predict the SCLC-P subtype. AC: Adenocarcinoma. AdenoSq: Adenosquamous carcinoma. LC: Large cell carcinoma. LCNEC: Large cell neuroendocrine carcinoma. SCLC: Small cell lung cancer. SqCC: Squamous cell carcinoma.

FIG. 9 shows the prediction of subtypes using the GSE56044 dataset using large models (including 15,000 Methylation markers) and reduced panels consisting of two markers (upper heatmap) and three markers (middle heatmap) using models to specifically predict the SCLC-I subtype. AC: Adenocarcinoma. AdenoSq: Adenosquamous carcinoma. LC: Large cell carcinoma. LCNEC: Large cell neuroendocrine carcinoma. SCLC: Small cell lung cancer. SqCC: Squamous cell carcinoma.

FIGS. 10A-10C show assessment of methylation sites for their suitability in a liquid biopsy assay. Methylation levels were compared between groups and also compared to blood cells and other potential sources of cfDNA as well as real liquid biopsy samples in various cancers.

FIG. 11 shows methylation levels of markers selected to be specific for SCLC and other HGNEC. The data from the GSE60644 dataset was used consisting of 124 lung cancer samples. AC: Adenocarcinoma. AdenoSq: Adenosquamous carcinoma. LC: Large cell carcinoma. LCNEC: Large cell neuroendocrine carcinoma. SCLC: Small cell lung cancer. SqCC: Squamous cell carcinoma.

FIG. 12 shows methylation levels of markers selected to be specific for SCLC/HGNEC. The data from the pan cancer TCGA dataset was used. There are no SCLC samples in this dataset.

FIG. 13 shows correlation of methylation sites with drug response. The drug tested (IC₅₀) is marked on the x-axis while the methylation beta value derived for each methylation site is shown on the y-axis. A trendline calculated by a linear model is added to highlight the association.

FIG. 14 shows a diagram of the analytical strategy for the selection of the respective markers following RRBS analysis described in Example 4. The markers in the final selection were further validated to be suitable in a liquid biopsy assay.

FIG. 15 shows classification of cell lines using a logistic regression model trained on the selected methylation sites based on the RRBS analysis described in Example 4. In the top row of the heatmap, the subtype classification based on the RNA signature is highlighted. The lower four rows highlight the prediction probability (Prob) for the four different SCLC subtypes (A=SCLC-A, N=SCLC-N, P=SCLC-P, I=SCLC-I). All cell lines were correctly classified.

FIG. 16 shows classification of patient-derived xenograft samples using a logistic regression model trained on the selected methylation sites from cell lines based on the RRBS analysis described in Example 4. In the top row of the heatmap, the subtype classification based on the RNA signature is highlighted. The lower four rows highlight the prediction probability (Prob) for the four different SCLC subtypes (A=SCLC-A, N=SCLC-N, P=SCLC-P, I=SCLC-I).

FIGS. 17A and 17B show a distribution of methylation sites considered suitable for a liquid biopsy across the human genome. Each chromosome is shown individually, and the sites derived from microarray analysis described in Examples 1 and 2 and from RRBS analysis described in Example 4 are highlighted according to their coordinates on the respective chromosome.

FIGS. 18A and 18B show a distribution of methylation sites that were derived from the xenograft analysis described in Example 4 and were considered useful for a liquid biopsy across the human genome. Each chromosome is shown individually and the methylation sites from RRBS analysis are highlighted according to their coordinates on the respective chromosome.

FIGS. 19A-19C show detection and classification of SCLC. FIG. 19A. Predictive models were generated to classify SCLC based on RNAseq and consensus of several combined predictive models is shown. A subtype was called when the consensus >0.5, else a sample was called equivocal. If two subtypes had consensus >50%, the sample was called both subtype (top annotation row). In addition, the expression of the three transcription factor ASCL1 (for SCLC-A), NEUROD1 (for SCLC-N) and POU2F3 (for SCLC-P) is shown with good concordance to the predictions. FIG. 19B. While the SCLC-A and SCLC-N subtypes showed a strong expression of neuroendocrine genes (NE) in RNAseq, non-neuroendocrine genes (NonNE) were enriched in the SCLC-I and SCLC-P subtypes.

FIG. 19C. The Epithelial-to-Mesenchymal (EMT) score calculated for the four subtypes highlights profound differences with a more mesenchymal phenotype in SCLC-I and a more epithelial phenotype in SCLC-P and SCLC-A. The box plot is highlighting the median as well as the 25^{th} and 75^{th} percentile in the box extended by 1.5× the IQR with bars.

FIGS. 20A-20G show subtype-specific DNA methylation in SCLC. FIG. 20A. DNA methylation was assessed using reduced-representation bisulfite sequencing (RRBS) and DNA methylation was averaged per sample and subtype over 100 kbp bins (represented by a dot) and the rolling average over 500 bins (=50 mbp) is highlighted in the FFPE tumor samples. DNA methylation sites were further annotated by their association with genes (including promoters, exons, introns, 1-5 kb upstream, 5UTRs, intergenic, 3UTRs, first exons, intron-exon boundaries & exon-intron boundaries) and the number of hypermethylated regions (>90% DNA methylation; FIG. 20B) per subtype and hypomethylated regions (<10% DNA methylation; FIG. 20C) is shown. Furthermore, regions which are associated with one of the four subtypes (AUROC >0.8) are highlighted in FIGS. 20D-G for SCLC-A (FIG. 20D), SCLC-N (FIG. 20E), SCLC-P (FIG. 20F) and SCLC-I (FIG. 20G).

FIGS. 21A-21C show DNA methylation-based subtyping in SCLC. FIG. 21A. A classifier using predictive models was created to predict the SCLC subtypes in FFPE samples using DNA methylation (SCLC-DMC). A subtype was called when the consensus >0.5, else a sample was called equivocal (top annotation row). Classification was compared to the RNA-based classification (top annotation row). FIG. 21B. Global DNA methylation in cfDNA of matched samples per subtype. FIG. 21C. Prediction of subtypes in cfDNA of matched samples using the SCLC-DMC.

FIGS. 22A-22D show comparison of IC50 values for the BCL2i ABT-737 (FIG. 22A) and the AURKi CYC-116 (FIG. 22B) between cell lines assigned to SCLC-A and SCLC-N using SCLC-DMC. Clinical outcome depending on classification method used. Overall survival of ES-SCLC patients stratified by classification using the RNAseq signature or SCLC-DMC for SCLC-A (FIG. 22C) and SCLC-N(FIG. 22D). The box plot is highlighting the median as well as the 25^{th} and 75^{th} percentile in the box extended by 1.5× the IQR with bars.

FIG. 23 shows an overview on samples used in the clinical cohort. The Size (assessed by visual inspection during extraction), whether macro dissection prior to extraction was performed as well as the finally attributed subtypes are highlighted on top. In addition the concentration of extracted RNA as well as the DV200, which is defined by the percentage of RNA fragments with a length >200 bp is shown together with the information whether the sample was used in RNAseq. Furthermore, the DNA concentration of the extracted DNA together with the information if the samples was used in RRBS DNA methylation analysis is shown. All samples were profiled by qPCR and the expression of the three transcription factors, ASCL1, NEUROD1 and POU2F3 is highlighted in the bottom, normalized to GAPDH expression.

FIGS. 24A-24C show correlation analysis of RNAseq and qPCR. Due to the low sample input and high DV200 leading to low mapping rates, the results of the RNAseq was correlated to the qPCR results for ASCL1 (FIG. 24A), NEUROD1 (FIG. 24B) and POU2F3 (FIG. 24C). The correlation coefficient using Pearson correlation as well as the p-value is highlighted for each correlation.

FIGS. 25A and 25B show expression of immune related genes in SCLC clinical specimen. FIG. 25A. Expression of different HLA genes ordered by different subtypes. Expression of HLA genes is enriched in SCLC-P and SCLC-I. FIG. 25B. Expression of different immune genes ordered by different subtypes. The expression of immune-related genes are enriched in SCLC-P and SCLC-I highlighting a more immunogenic subtype.

FIGS. 26A-26D show global DNA methylation across cell lines and xenograft models. DNA methylation was assessed using reduced-representation bisulfite sequencing (RRBS) or Methylation arrays and DNA methylation was averaged per sample and subtype over 100 kbp bins (represented by a dot) and the rolling average over 500 bins (=50 mbp) is highlighted for cell lines from the investigators provided analyzed using RRBS (FIG. 26A), cell lines derived from the NIC SCLC cell miner project (Tlemsani et al., 2020, Cell Reports 33, 108296) using the illumina EPIC array (FIG. 26B), cell lines derived from the GDSC dataset (Iorio et al., 2016, Cell, 166(3), 740-754) using the illumina 450K array (FIG. 26C), and in-house samples of patient-derived xenografts using RRBS (FIG. 26D).

FIGS. 27A-27D show a comparison of predictive DNA methylation sites between FFPE clinical samples and cell lines. DNA methylation sites that were highly associated with one of the four subtypes in cell lines and FFPE samples (AUROC >0.8) were selected and filtered for DNA methylation sites for which information was present in both datasets. DNA methylation sites which are shared among both sample types or which are unique to each are shown for SCLC-A (FIG. 27A), SCLC-N (FIG. 27B), SCLC-P (FIG. 27C) and SCLC-I (FIG. 27D).

FIGS. 28A-28C show DNA methylation sites of the three transcription factors per subtype. The four subtypes are defined by the expression of the three transcription factors ASCL1 (SCLC-A), NEUROD1 (SCLC-N) and POU2F3 (SCLC-P) as well as by absence of the three (SCLC-I). Consequently, differences in DNA methylation for different regions for each of the three transcription factors are shown per subtype for ASCL1 (FIG. 28A), NEUROD1 (FIG. 28B) and POU2F3 (FIG. 28C). The box plot is highlighting the median as well as the 25th and 75th percentile in the box extended by 1.5× the IQR with bars. Significance was calculated using two-sided student's t-test and provided for each comparison above the boxplots.

FIG. 29 shows DNA methylation sites per region per subtype. DNA methylation sites were analyzed according to their region next to a respective gene to allow further functional assessment. For each of the four subtypes, the total DNA methylation level for each of the regions is highlighted. The box plot is highlighting the median as well as the 25th and 75th percentile in the box extended by 1.5× the IQR with bars. Significance is calculated using two-sided student's t-test and provided for each comparison above the boxplots.

FIG. 30 shows classification of cell lines using the SCLC-DMC. The same model that was trained on the clinical specimens was used to predict the subtype in 59 cell lines. The heatmap highlights the consensus prediction of the models.

FIGS. 31A and 31B show global DNA methylation across cfDNA samples. DNA methylation was assessed using reduced-representation bisulfite sequencing (RRBS) and DNA methylation was averaged per sample and subtype over 100 kbp bins. FIG. 31A. To assess the differences of cfDNA to FFPE DNA, matched samples were selected and the differences for each bin is highlighted in (represented by a dot) and the rolling average over 500 bins (=50 mbp) for each sample individually. FIG. 31B. The global methylation pattern in cfDNA is highlighted for each sample individually likewise with each dot representing a bin of 100 kbp and the line the rolling average of 500 bins. In addition to comparing the results to cfDNA from healthy donors, data was retrieved from (Van Paemel et al., 2021, Epigenetics, 33074045) and is highlighted by average across all provided samples.

### DETAILED DESCRIPTION

Using mRNA gene expression patterns, tumors from SCLC patients can be classified into four major subtypes of SCLC. Three of them are defined by differential expression of the transcription factors ASCL1 (SCLC-A), NEUROD1 (SCLC-N), and POU2F3 (SCLC-P), and a fourth group is characterized for having high expression of inflammatory-related genes (SCLC-I). Importantly, these subtypes have distinct therapeutic vulnerabilities and show differential response patterns to standard of care and investigation agents. Similar subgroups also exist in other HGNEC, such as Large Cell Neuroendocrine Carcinoma of the lung. Certain methods for such classification and treatment of SCLC and HGNEC are described in U.S. Patent Application Publication 2021/0062274 and Gay C M, et al., Cancer Cell. 2021 Jan. 5:S1535-6108(20)30662-0, each incorporated by reference herein in its entirety.

The present disclosure is based, at least in part, on the discovery that certain DNA methylation sites can be used to identify and classify small cell lung cancer and its subtypes (SCLC-A, SCLC-N, SCLC-P, and SCLC-I), and can also be used to inform treatment decisions for SCLC patients. Aspects of the present disclosure describe analysis of DNA methylation from tumor tissue, blood, or other sources for classification and treatment of SCLC and HGNEC. Certain aspects are directed to methods for identifying a patient as having SCLC-A, SCLC-N, SCLC-P, or SCLC-I based on analysis of methylation sites of tumor DNA from the subject including, for example, the methylation sites of Table 1, 2, 3, 4, 5, 6, 7, 8, 9, 10, 15, 16, 17, 18, 19, 20, 21, 22, 23, 24, 26, 27, 28, 29, and/or 30. For example, a subject may be identified as having a particular SCLC subtype based on identifying differential methylation of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000 methylation sites (or any range or value derivable therein) of Table 1, 2, 3, 4, 5, 6, 7, 8, 9, 10, 15, 16, 17, 18, 19, 20, 21, 22, 23, 24, 26, 27, 28, 29, and/or 30. It is specifically contemplated that any combination of any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000 methylation sites (or any range or value derivable therein) of Table 1, 2, 3, 4, 5, 6, 7, 8, 9, 10, 15, 16, 17, 18, 19, 20, 21, 22, 23, 24, 26, 27, 28, 29, and/or 30 may be included in a method, composition, or kit of the present disclosure. It is specifically contemplated that any 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000 methylation sites (or any range or value derivable therein), or more, methylation sites of Table 1, 2, 3, 4, 5, 6, 7, 8, 9, 10, 15, 16, 17, 18, 19, 20, 21, 22, 23, 24, 26, 27, 28, 29, and/or 30 may be excluded in embodiments described herein.

Additional aspects are directed to methods for identifying a subject with cancer as having SCLC based on analysis of methylation sites of tumor DNA from the subject including, for example, the methylation sites of Table 13. For example, a subject may be identifying as having SCLC, and not as having a different cancer type, based on identification of differential methylation of one or more of the methylation sites of Table 13.

Further aspects relate to methods for treatment of SCLC or HGNEC based on analysis of methylation sites of tumor DNA from the subject including, for example, the methylation sites of Table 1, 2, 3, 4, 5, 6, 7, 8, 9, 10, 15, 16, 17, 18, 19, 20, 21, 22, 23, 24, 26, 27, 28, 29, and/or 30. For example, a subject may be treated for SCLC with a particular treatment based on identifying differential methylation of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000 of the methylation sites of Tables 1, 2, 3, 4, 5, 6, 7, 8, 9, 10, 15, 16, 17, 18, 19, 20, 21, 22, 23, 24, 26, 27, 28, 29, and/or 30. Example therapies useful in treatment of a particular SCLC subtype are described herein. It is specifically contemplated that 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000 methylation sites (or any range or value derivable therein), or more, methylation sites of Tables 1, 2, 3, 4, 5, 6, 7, 8, 9, 10, 15, 16, 17, 18, 19, 20, 21, 22, 23, 24, 26, 27, 28, 29, and/or 30 may be excluded in embodiments described herein.

### I. Treatment Methods

Aspects of the present disclosure include methods of treating a patient with small cell lung cancer (SCLC) or other high-grade neuroendocrine carcinoma (HGNEC). Certain aspects are directed to methods for treatment of a subject for SCLC, where the treatment is selected based on the SCLC subtype of the subject. As described herein, a subject may have SCLC, where the SCLC can be classified as one of four subtypes: SCLC-A, SCLC-N, SCLC-P, or SCLC-I.

In some embodiments, the subject is identified as having an SCLC subtype based on the expression or methylation status of ASCL1, NEUROD1, and POU2F3 in nucleic acid from cancer tissue from the subject. SCLC-A may be identified based on expression of ASCL1 and lack of expression of NEUROD1 or POU2F3. SCLC-N may be identified based on expression of NEUROD1 and lack of expression of either ASCL1 or POU2F3. SCLC-P may be identified based on expression of POU2F3 and lack of expression of either ASCL1 or NEUROD1. SCLC-I may be identified based on lack of expression of any of ASCL1, NEUROD1, and POU2F3.

In some embodiments, the subject is identified as having an SCLC subtype based on analysis of the methylation status of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Tables 1, 2, 3, 4, 5, 6, 7, 8, 9, 10, 15, 16, 17, 18, 19, 20, 21, 22, 23, 24, 26, 27, 28, 29, and/or 30 from tumor DNA from the subject. Analyses of each and every specific combination of the methylation sites of Tables 1, 2, 3, 4, 5, 6, 7, 8, 9, 10, 15, 16, 17, 18, 19, 20, 21, 22, 23, 24, 26, 27, 28, 29, and/or 30 are contemplated herein. It is specifically contemplated that any one or more of the methylation sites of Tables 1, 2. 3, 4, 5, 6, 7, 8, 9, 10, 15, 16, 17, 18, 19, 20, 21, 22, 23, 24, 26, 27, 28, 29, and/or 30 may be excluded in embodiments described herein. In some embodiments, tumor DNA is obtained or derived from a tissue sample from the subject. In some embodiments, tumor DNA is obtained or derived from a blood sample from a subject. In some embodiments, tumor DNA is obtained or derived from a plasma sample from a subject. In some embodiments, the tumor DNA is circulating tumor DNA (ctDNA).

In some embodiments, SCLC-A is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 2. Analyses of each and every specific combination of the methylation sites of Table 2 are contemplated herein. It is specifically contemplated that any one or more of the methylation sites of Table 2 may be excluded in embodiments described herein. In some embodiments, SCLC-A is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 methylation sites of Table 7. Analyses of each and every specific combination of the methylation sites of Table 7 are contemplated herein. It is specifically contemplated that any one or more of the methylation sites of Table 7 may be excluded in embodiments described herein. In some embodiments, SCLC-A is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29.30 methylation sites of Table 15. Analyses of each and every specific combination of the methylation sites of Table 15 are contemplated herein. It is specifically contemplated that any one or more of the methylation sites of Table 15 may be excluded in embodiments described herein. In some embodiments, SCLC-A is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, or 1340 methylation sites of Table 20. Analyses of each and every specific combination of the methylation sites of Table 20 are contemplated herein. It is specifically contemplated that any one or more of the methylation sites of Table 20 may be excluded in embodiments described herein. In some embodiments, SCLC-A is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 27. Analyses of each and every specific combination of the methylation sites of Table 27 are contemplated herein. It is specifically contemplated that any one or more of the methylation sites of Table 27 may be excluded in embodiments described herein. In some embodiments, SCLC-A is identified based on detection of differential methylation of cg00799539, cg04610718, cg10672201, cg17277939, cg11201256, cg07639982, cg01566028, cg06043710, cg01154505, cg09643186, cg03817675, cg00090674, cg02639667, cg22053861, cg14338051, cg00794178, cg16860004, cg04317756, cg01942646, cg11249931, cg08279731, cg05161074, cg00773370, cg07444408, cg08758185, cg27119612, cg22904437, cg04506569, cg04877165, and/or cg01610165. In some embodiments, SCLC-A is identified based on detection of differential methylation of chr17:74961036, chr17:74961013, chr18:59062159, chr19:13506705, chr9:134815629, chr21:41180000, chr9:93014411, chr9:114211349, chr9:134810271, chr19:3385734, chr6:51213948, chr18:27786347, chr6:168638645, chr9:134815657, chr9:134815611, chr16:84519934, chr20:20364625, chr5:172103442, chr20:22598951, chr16:85355101, chr9:134815628, chr20:20364629, chr19:511206, chr6:168149181, chr7:100122306, chr16:84519930, chr8:58989473, chr18:27786294, chr20:22557408, chr9:128177708, chr7:157633760, chr6:40349351, chr6:157229362, chr13:84927994, chr4:8101279, chr22:41667328, chr5:176843804, chr5:176529007, chr4:137426451, chr6:40349318, chr11:11578650, chr20:20364628, chr6:40349319, chr17:48190199, chr7:100122301, chr1:226589216, chr22:40022901, chr6:37078047, chr9:134765725, chr20:20364641, chr18:77020770, chr17:3695881, chr19:38253123, chr9:114255802, chr7:157017749, chr17:74823356, chr9:117355642, chr6:51213976, chr7:151045548, chr16:4537495, chr2:45274755, chr8:139674518, chr12:132134577, chr9:134885740, chr16:4537572, chr17:1961091, chr6:51213977, chr11:888785, chr9:134815626, chr13:110011941, chr19:13372294, chr16:84519955, chr20:20364626, chr20:20364640, chr17:57874806, chr6:168638634, chr7:14372215, chr22:50010360, chr14:56814573, chr13:112641259, chr7:146633722, chr7:157633743, chr21:43758530, chr6:168538915, chr5:176843791, chr2:46983311, chr2:239300749, chr7:157998148, chr6:34128215, chr13:112106712, chr16:3999700, chr9:114172063, chr9:134792142, chr11:11578688, chr7:157833048, chr1:151346323, chr21:38661697, chr1:226588954, chr20:6232329, chr9:134792130, chr9:134785070, chr9:134815627, chr9:134592825, chr16:84519980, chr7:100122290, chr11:11973687, chr19:38253193, chr14:44405368, chr16:4260161, chr2:28411073, chr1:50214861, chr16:84519943, chr22:19933408, chr11:68833022, chr7:73985377, chr9:93014375, chr9:114183920, chr16:4260632, chr9:133558991, chr6:168638701, chr7:157658598, chr19:13506729, chr19:13483181, chr16:84519942, chr11:76784947, chr11:65551424, chr7:2193699, chr5:179258981, chr14:99518884, chr18:27786301, chr11:64056580, chr6:169251928, chr9:134486435, chr12:132134595, chr16:84519935, chr21:41179996, chr14:105392911, chr7:40330569, chr13:111622152, chr2:98823202, chr17:29972557, chr9:134815918, chr1:213905614, chr7:2607131, chr4:8101234, chr12:103012503, chr14:44262090, chr6:82363058, chr9:114170339, chr6:168138608, chr12:11856447, chr19:13634667, chr9:93722421, chr9:114172123, chr17:29565848, chr11:64353390, chr9:90908530, chr16:3999757, chr11:65551425, chr9:114306707, chr19:2354863, chr9:136289024, chr6:107633714, chr7:157017791, chr6:168496984, chr3:45819148, chr18:77102188, chr12:105784446, chr17:29972550, chr11:119702010, chr5:1316124, chr7:73966925, chr3:53702765, chr11:118924054, chr19:51077543, chr17:81246338, chr12:3077928, chr12:130460997, chr7:157963798, chr12:130483815, chr7:157963771, chr14:64942593, chr18:59203056, chr19:13463517, chr20:20364646, chr7:157987469, chr2:216380208, chr20:16806905, chr17:74961041, chr3:176190636, chr20:22652824, chr6:37078083, chr14:44405319, chr18:72677631, chr9:136686592, chr2:216986073, chr2:10040203, chr21:38636406, chr9:134824994, chr12:119158214, chr7:5795946, chr22:19933350, chr9:134785048, chr13:111559772, chr16:88640994, chr17:29972549, chr6:168130574, chr6:168130602, chr9:114183394, chr9:120326115, chr3:177516507, chr7:110084403, chr22:50010361, chr18:76681573, chr7:158485004, chr21:38661630, chr15:53659351, chr19:13347369, chr19:39163809, chr17:48620272, chr18:71652249, chr6:168667391, chr10:133309889, chr1:241210311, chr19:46770637, chr20:22556743, chr2:47227055, chr7:150974698, chr20:22653042, chr14:73228468, chr19:18422970, chr21:40144935, chr16:85355024, chr19:13575256, chr9:134690692, chr6:82363052, chr7:122307220, chr7:157633801, chr1:43439683, chr16:64387, chr2:216986079, chr9:134885778, chr4:128846433, chr1:43550363, chr3:9180988, chr19:511219, chr20:62711715, chr11:47327260, chr6:51214001, chr5:10652237, chr4:6944534, chr21:38599607, chr6:168440545, chr1:18794157, chr8:52150618, chr9:133662377, chr16:4537619, chr19:39307494, chr19:55254005, chr12:130535047, chr5:82804, chr6:34128233, chr12:11856451, chr19:13506452, chr17:77452817, chr6:109290309, chr21:38661664, chr10:133309857, chr21:34667592, chr7:100477920, chr20:38294975, chr21:41648785, chr21:43758525, chr9:134822835, chr16:3728390, chr16:29604945, chr1:41727346, chr21:45032479, chr1:39954061, chr17:29972554, chr6:151809197, chr12:111287675, chr19:13463510, chr8:52150574, chr19:13014442, chr4:8164496, chr7:8417325, chr7:157673872, chr2:107461969, chr9:36781051, chr4:138912435, chr7:157594362, chr2:237733847, chr20:22652808, chr14:64942648, chr16:4260588, chr7:157603987, chr7:157540317, chr13:111515678, chr17:67488327, chr7:73990185, chr10:43849791, chr17:1961149, chr6:82585382, chr1:224184849, chr2:182053988, chr21:41508507, chr15:85942771, chr10:78139388, chr9:126420967, chr15:67806290, chr7:157963797, chr9:113593968, chr7:157875450, chr7:157875495, chr14:64942637, chr19:55254025, chr1:205447752, chr16:84519956, chr9:134797123, chr18:63207225, chr9:134772536, chr8:38495018, chr13:112107447, chr5:100864671, chr14:91418510, chr9:134768753, chr19:13245332, chr11:47327256, chr8:42275934, chr12:103165897, chr18:38714972, chr7:157824560, chr14:75978696, chr15:40873466, chr4:104663323, chr5:1316150, chr4:189096959, chr9:134750430, chr13:98405890, chr9:134825870, chr16:52621839, chr17:62653135, chr9:134881575, chr7:157633794, chr9:114170237, chr18:77020828, chr2:216840164, chr2:181684626, chr9:134819274, chr21:34743983, chr17:57874702, chr22:28923959, chr21:44698714, chr11:65551324, chr9:113593972, chr7:2193759, chr6:168440498, chr8:141264221, chr16:4260115, chr16:4537502, chr20:20365491, chr1:7491237, chr20:20364647, chr11:31893506, chr19:41704632, chr20:17068564, chr5:6543207, chr19:41323849, chr2:85733778, chr7:157685012, chr11:75240135, chr6:168315218, chr14:64633473, chr11:20056293, chr14:44379797, chr16:4537618, chr7:157864088, chr10:133309864, chr14:88256419, chr16:19425106, chr6:168355227, chr9:134765734, chr6:168453036, chr21:41490949, chr7:73989381, chr12:3077894, chr1:53462420, chr9:113593928, chr10:133309740, chr7:101666174, chr11:14974476, chr5:100944956, chr20:22557482, chr18:74782855, chr19:3656386, chr7:26397761, chr19:13463491, chr22:38087144, chr9:130535984, chr9:134892203, chr13:107429178, chr19:38253194, chr17:82078952, chr1:1174126, chr1:232307467, chr6:14499964, chr16:4260566, chr16:85286422, chr17:74006568, chr7:101707772, chr6:168496991, chr18:27786339, chr13:113793557, chr19:47209566, chr2:113325550, chr5:176529022, chr8:52150549, chr9:113594013, chr9:134490841, chr7:1620862, chr9:134179005, chr15:63053313, chr12:124617614, chr21:45032480, chr13:113723025, chr7:150945169, chr12:6961918, chr19:39307859, chr16:89948104, chr19:8153491, chr20:20365487, chr20:38294974, chr3:73790643, chr6:153237290, chr6:168130594, chr7:2524564, chr9:134785301, chr9:136367817, chr19:38253176, chr19:406561, chr1:52633297, chr9:93819344, chr11:76784948, chr14:36524358, chr13:112106739, chr6:157228665, chr7:157953525, chr10:101780858, chr12:110942244, chr6:43578006, chr2:51027486, chr7:157685871, chr21:45032448, chr21:45032452, chr7:157017829, chr11:65551413, chr7:157544388, chr6:50845964, chr5:138650055, chr1:229147368, chr9:134782536, chr9:134316337, chr12:124259764, chr11:11578685, chr20:17068489, chr20:20365493, chr22:28923940, chr22:28923966, chr1:153536136, chr9:93861253, chr11:64540473, chr2:229672283, chr7:157633759, chr9:134534796, chr9:114170276, chr19:5950505, chr1:32885121, chr12:6962651, chr18:27786338, chr18:63207261, chr19:41704681, chr8:141289585, chr11:124895535, chr17:57874844, chr11:75335191, chr18:71337086, chr2:46983412, chr1:212589163, chr20:48751499, chr4:78056611, chr3:176814702, chr17:81246299, chr2:3940818, chr12:98238503, chr9:114170300, chr11:924647, chr19:55254024, chr2:208703401, and/or chr20:22598941, including any combination thereof.

In some embodiments, SCLC-N is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 3. Analyses of each and every specific combination of the methylation sites of Table 3 are contemplated herein. It is specifically contemplated that any one or more of the methylation sites of Table 3 may be excluded in embodiments described herein. In some embodiments, SCLC-N is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 methylation sites of Table 8. Analyses of each and every specific combination of the methylation sites of Table 8 are contemplated herein. It is specifically contemplated that any one or more of the methylation sites of Table 8 may be excluded in embodiments described herein. In some embodiments, SCLC-N is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 methylation sites of Table 16. Analyses of each and every specific combination of the methylation sites of Table 16 are contemplated herein. It is specifically contemplated that any one or more of the methylation sites of Table 16 may be excluded in embodiments described herein. In some embodiments, SCLC-N is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 650, or 695 methylation sites of Table 21. It is specifically contemplated that any one or more of the methylation sites of Table 21 may be excluded in embodiments described herein. In some embodiments, SCLC-N is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 28. It is specifically contemplated that any one or more of the methylation sites of Table 28 may be excluded in embodiments described herein. In some embodiments, SCLC-N is identified based on detection of differential methylation of cg20505457, cg04220881, cg14755690, cg19798702, cg03920522, cg12187160, cg02531277, cg18780243, cg19028997, cg02314596, cg16498113, cg19513004, cg17401435, cg25043538, cg00648184, cg10414350, cg09309024, cg06440275, cg01431993, cg11528849, cg16640855, cg00369376, cg02836529, cg20806345, cg22976218, cg13538006, cg11887270, cg23731742, cg02622825, cg00284708, cg05234415, cg24657047, cg23994112, cg08399017, cg14647703, cg13969327, cg05214130, cg08573199, cg09621610, cg13417891, cg02902261, cg00678971, cg02243624, cg13808641, cg15076368, and/or cg11964216. In some embodiments, SCLC-N is identified based on detection of differential methylation of chr1:54356529, chr1:109296245, chr1:109296247, chr1:220653171, chr1:220653195, chr11:35853956, chr11:64154503, chr11:64154571, chr11:64154574, chr12:108792675, chr17:72440313, chr17:72440361, chr2:223901322, chr20:19919227, chr3:52468694, chr5:160245925, chr6:44261803, chr8:42275934, chr9:131721310, chr9:131721341, chr9:131721355, chr9:131721389, chr2:216909627, chr20:19919173, chr9:131721356, chr22:50276667, chr17:72440360, chr6:37537738, chr1:220653165, chr1:220653180, chr8:140270696, chr12:108792684, chr19:3656274, chr1:54356527, chr19:47650943, chr7:149261799, chr2:216380208, chr17:72440372, chr9:131721340, chr9:131721342, chr2:216380274, chr11:67493309, chr2:65583301, chr5:160245988, chr3:52468622, chr17:74823299, chr1:160362850, chr12:31009628, chr14:64633473, chr18:59049918, chr20:63878908, chr5:16538225, chr6:19719869, chr6:34128215, chr6:34128233, chr1:109296235, chr3:52522225, chr8:134941130, chr7:149261769, chr19:3656352, chr2:85733778, chr4:850796, chr3:126013960, chr19:3655899, chr22:44049853, chr9:120242580, chr1:41657833, chr1:220653181, chr11:8239208, chr2:65583300, chr5:16538207, chr6:34128222, chr9:131721343, chr7:149261778, chr14:105178678, chr12:31009602, chr1:28869236, chr1:220653223, chr7:44254153, chr6:43475701, chr15:68347588, chr20:49345755, chr5:160245950, chr19:3656298, chr19:3655920, chr20:49345760, chr6:34128216, chr3:52468649, chr1:944700, chr1:54356503, chr1:54356526, chr1:54356528, chr2:216909691, chr5:160245949, chr21:41490949, chr17:50076230, chr2:25702014, chr19:3655770, chr9:120242557, chr16:85439239, chr1:53320062, chr5:16538206, chr6:19719865, chr11:855989, chr19:3655921, chr6:107633786, chr19:14182660, chr19:48350598, chr3:52468650, chr16:10622418, chr3:52468678, chr11:20055879, chr2:216909621, chr6:34128193, chr16:85287295, chr1:53320061, chr7:100890535, chr12:48675723, chr2:25701951, chr20:63835624, chr4:76612797, chr6:43475769, chr3:16132696, chr11:66866493, chr12:31009572, chr1:226588954, chr16:66960251, chr19:3656369, chr17:4809230, chr7:157018053, chr20:11271495, chr17:82001560, chr17:82001559, chr2:85770228, chr20:49345801, chr21:41458778, chr6:34128249, chr11:44963230, chr1:944730, chr12:108792716, chr22:44049787, chr3:52522201, chr4:6308154, chr2:85733793, chr19:12953781, chr1:43550353, chr21:41490965, chr12:108792685, chr3:16132702, chr1:15723882, chr1:54356555, chr12:130484433, chr17:4809161, chr6:19719861, chr6:26385134, chr6:168130602, chr19:13913675, chr16:85287080, chr11:888785, chr19:3655898, chr1:1921168, chr2:200087052, chr19:2354832, chr11:129699789, chr1:28869229, chr19:18422970, chr1:944737, chr22:44049781, chr20:25239829, chr9:120242569, chr6:148509695, chr1:153536157, chr9:120242617, chr12:31009603, chr18:62333487, chr8:140270737, chr19:3655888, chr17:82001529, chr7:102424329, chr2:200087071, chr16:85287095, chr11:855972, chr11:855981, chr9:120242622, chr19:3656250, chr20:19836028, chr1:944699, chr17:2418940, chr3:185397845, chr7:44151120, chr11:35853892, chr12:123253744, chr20:49345788, chr6:34128234, chr7:750747, chr2:205764439, chr18:59050139, chr5:172103442, chr9:120242544, chr1:28869207, chr16:11997774, chr8:134499858, chr17:29158240, chr1:54356559, chr6:34545466, chr8:134941060, chr10:101780858, chr19:12953855, chr3:53702770, chr12:56997304, chr6:34128223, chr18:77053845, chr20:22557408, chr6:110414306, chr9:136013178, chr1:230759612, chr6:44261814, chr6:42963345, chr9:95738191, chr16:85355101, chr19:2354863, chr1:53320016, chr16:70318894, chr16:85696894, chr17:82001584, chr2:200087032, chr7:128075308, chr1:32885121, chr21:41665312, chr7:128881062, chr6:169160984, chr3:196992182, chr6:40349319, chr7:44151126, chr1:54356214, chr7:5276684, chr19:38253104, chr1:204921255, chr1:224184849, chr11:48025532, chr5:16538197, chr1:153536113, chr1:153536123, chr9:124820965, chr21:41639350, chr9:113901422, chr1:11965505, chr6:168638645, chr17:29582881, chr3:71074388, chr9:134486435, chr19:48350605, chr19:3656178, chr11:118901571, chr1:43550383, chr12:123253763, chr16:30030208, chr21:41648785, chr9:91810447, chr10:79313956, chr6:169190341, chr16:85436880, chr1:1921165, chr1:205447760, chr1:153536136, chr1:153536146, chr11:19730746, chr3:141093639, chr2:159228539, chr21:17508899, chr11:66866541, chr17:66830069, chr20:49345770, chr20:63835571, chr9:127200650, chr19:4055918, chr9:131810494, chr19:38253123, chr1:60903781, chr22:20014786, chr1:25771857, chr17:67652635, chr9:93166951, chr13:73218843, chr7:136045406, chr14:73228468, chr17:50831246, chr7:30171980, chr17:67652633, chr1:28869065, chr13:113818528, chr2:200087031, chr2:200087051, chr6:168517547, chr6:35534674, chr6:35534714, chr6:148509713, chr19:3656386, chr11:19832444, chr13:112775959, chr22:19632813, chr5:760546, chr19:3950318, chr1:60903756, chr7:8070969, chr16:70730157, chr20:49345761, chr3:45819148, chr6:19719834, chr6:34128254, chr6:34128257, chr6:168130634, chr1:15723893, chr16:85287162, chr7:1815269, chr9:93166950, chr16:85287116, chr17:67652568, chr12:123253794, chr16:85287098, chr18:77077978, chr19:46714207, chr3:53702765, chr7:3998211, chr9:92729620, chr16:85436709, chr11:66866538, chr22:44248796, chr6:34545471, chr7:102424320, chr17:602149, chr20:22557482, chr6:3444679, chr17:57707866, chr16:11654442, chr19:3655812, chr13:109334658, chr1:43550373, chr2:73080399, chr9:131810462, chr1:227920141, chr1:153536121, chr20:50035301, chr20:38700627, chr21:41553133, chr21:41648778, chr6:168130595, chr6:168539582, chr7:5276649, chr21:40144935, chr19:3656261, chr3:139718818, chr1:10625828, chr3:185397835, chr7:110084403, chr5:176843791, chr6:40349351, chr9:113901566, chr21:41179996, chr4:528986, chr12:48675786, chr19:13634667, chr6:168130574, chr19:3656190, chr1:202168439, chr12:124428932, chr6:34545461, chr7:3998240, chr3:127304410, chr16:89847327, chr1:226638169, chr1:181128695, chr1:54356181, chr12:123253764, chr11:64056580, chr3:47236186, chr16:85436855, chr11:888597, chr19:4010290, chr1:53320091, chr1:205257276, chr19:4010298, chr21:41648770, chr7:122306890, chr20:11271554, chr4:7911302, chr1:28869195, chr16:2455154, chr5:176843804, chr7:5276677, chr1:220653196, chr11:888641, chr19:38253076, chr4:1858204, chr8:38495138, chr3:141093636, chr1:31627824, chr12:48675768, chr2:190034714, chr6:168638690, chr19:3656351, chr12:131864061, chr9:89591607, chr17:74823356, chr16:85287017, chr16:88887476, chr2:96174325, chr6:3444622, chr1:5917479, chr1:1240684, chr12:130483799, chr13:110123705, chr6:168496991, chr8:53733503, chr7:105689129, chr2:113974997, chr12:591924, chr15:65333873, chr3:194305584, chr19:4059381, chr18:59049874, chr1:224184822, chr11:76175216, chr6:168130594, chr19:3655954, chr19:34999970, chr6:26614205, chr10:78139388, chr11:122984321, chr19:3656172, chr1:43550344, chr12:129694731, chr19:3655828, chr7:151045548, chr12:130483815, chr5:78519838, chr11:888359, chr1:205032318, chr9:120242579, chr1:16486605, chr1:20348597, chr2:43203382, chr6:168130603, chr17:3695871, chr11:19714522, chr16:85439179, chr7:122306891, chr9:130233164, chr2:47020392, chr19:3655938, chr8:58989422, chr17:80001489, chr2:5530198, chr19:3656393, chr7:1815299, chr21:43860194, chr6:168355227, chr1:20348596, chr7:5478011, chr2:200087040, chr19:48371166, chr16:22288921, chr19:12953833, chr11:888360, chr5:16538196, chr1:9251757, chr11:924646, chr12:131864094, chr2:25701937, chr6:168538915, chr9:127636823, chr1:22809032, chr17:29065994, chr6:168517537, chr4:6308178, chr1:61198659, chr20:11271453, chr7:44151141, chr19:18745096, chr7:30172111, chr6:34433316, chr12:119869919, chr13:110123752, chr17:29582888, chr18:36468445, chr19:4010297, chr19:39307494, chr20:63835620, chr21:41375919, chr5:6859175, chr6:37537680, chr7:100122236, chr9:93959419, chr9:91810580, chr17:73726103, chr1:226589216, chr9:23452269, chr6:42963307, and/or chr8:140321186, chr16:85287074, including any combination thereof.

In some embodiments, SCLC-P is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 4. Analyses of each and every specific combination of the methylation sites of Table 4 are contemplated herein. It is specifically contemplated that any one or more of the methylation sites of Table 4 may be excluded in embodiments described herein. In some embodiments, SCLC-P is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, or 93 methylation sites of Table 9. Analyses of each and every specific combination of the methylation sites of Table 9 are contemplated herein. It is specifically contemplated that any one or more of the methylation sites of Table 9 may be excluded in embodiments described herein. In some embodiments, SCLC-P is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 methylation sites of Table 17. Analyses of each and every specific combination of the methylation sites of Table 17 are contemplated herein. It is specifically contemplated that any one or more of the methylation sites of Table 17 may be excluded in embodiments described herein. In some embodiments, SCLC-P is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 989 methylation sites of Table 22. Analyses of each and every specific combination of the methylation sites of Table 22 are contemplated herein. It is specifically contemplated that any one or more of the methylation sites of Table 22 may be excluded in embodiments described herein. In some embodiments, SCLC-P is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 29. Analyses of each and every specific combination of the methylation sites of Table 29 are contemplated herein. It is specifically contemplated that any one or more of the methylation sites of Table 29 may be excluded in embodiments described herein. In some embodiments, SCLC-P is identified based on detection of differential methylation of cg27232389, cg03020852, cg11691710, cg14197123, cg17885507, cg03517570, cg13297671, cg18795320, cg03075214, cg06832246, cg05683632, cg00704369, cg22171098, cg09934399, cg02817764, cg24677222, cg16770048, cg14701925, cg04182076, cg18969798, cg03803789, cg13240089, cg17758792, cg06923861, cg07956003, cg08275278, cg04983681, cg17728697, cg09472222, cg02220284, cg05366160, cg13705753, cg06839854, cg02675973, cg05395645, cg25601286, cg11701604, cg07825782, cg00600617, cg16054907, cg01489441, cg15564226, cg10026427, cg01149239, cg04479757, cg11019743, cg13207778, cg25296465, cg03387092, cg14175330, cg05999426, cg19011089, cg14313328, cg18529415, cg04563108, cg25874150, cg16318349, cg13081156, cg14088354, cg03264729, cg01580044, cg07313835, cg11597418, cg26855672, cg03356778, cg05216984, cg02593168, cg20314331, cg26797073, cg15873449, cg18983310, cg08265811, cg22340762, cg00618183, cg12452539, cg15786168, cg06669752, cg24136932, cg24272324, cg04619304, cg01772824, cg13921012, cg06542565, cg10288437, cg10609655, cg07262682, cg21289124, cg07325342, cg14211075, cg00719067, cg05554594, cg08368885, and/or cg21876001. In some embodiments, SCLC-P is identified based on detection of differential methylation of chr1:11032833, chr1:15207848, chr1:15945017, chr1:17026179, chr1:25741499, chr1:27551002, chr1:32327585, chr1:42682988, chr1:52365896, chr1:56552275, chr1:116407596, chr1:147829076, chr1:156538486, chr1:156750879, chr1:164576909, chr1:197915932, chr1:204289083, chr1:204571233, chr1:212618744, chr1:212618752, chr1:212638013, chr1:231162869, chr1:231162872, chr1:231162880, chr10:14365352, chr10:55631088, chr10:55631097, chr10:97713353, chr10:97713362, chr10:123017613, chr11:63997490, chr11:65536690, chr11:75410397, chr11:77750252, chr11:126313324, chr12:433809, chr12:7291128, chr12:57128650, chr12:67389978, chr12:75861004, chr12:81759542, chr12:85489437, chr12:101309330, chr12:103993054, chr12:104599362, chr12:104599370, chr12:105977083, chr12:110245057, chr12:110245058, chr12:110245093, chr12:111841988, chr12:120456258, chr12:122032753, chr12:122032775, chr12:124497504, chr12:124524949, chr12:124631301, chr13:20345144, chr14:89701670, chr14:105392906, chr14:105392911, chr15:43746172, chr15:58701617, chr15:74789232, chr15:74789276, chr15:74789281, chr16:234790, chr16:234826, chr16:648840, chr16:1498961, chr16:1746192, chr16:2075993, chr16:2964040, chr16:4767447, chr16:8621572, chr16:28193303, chr16:28863649, chr16:29906931, chr16:30125736, chr16:30973692, chr16:66696829, chr16:68341637, chr16:85611879, chr17:21665928, chr17:29576080, chr17:34252590, chr17:43187274, chr17:48932449, chr17:49816143, chr17:63383824, chr17:63383830, chr17:63739476, chr17:64661931, chr17:68205724, chr17:72165644, chr17:76420086, chr17:77439476, chr17:77439513, chr17:81248481, chr17:81248517, chr18:5543758, chr18:5543760, chr18:5543767, chr18:5543774, chr18:14907472, chr18:28175723, chr18:28175730, chr18:28175734, chr18:28175743, chr18:57574427, chr19:807444, chr19:811705, chr19:991153, chr19:991161, chr19:1633524, chr19:1993008, chr19:2115209, chr19:5618219, chr19:11483864, chr19:11483872, chr19:11483877, chr19:11483882, chr19:11483887, chr19:11483902, chr19:11483907, chr19:11483912, chr19:14206288, chr19:14559510, chr19:16628375, chr19:17209718, chr19:17209725, chr19:17209733, chr19:17209752, chr19:18778224, chr19:35721194, chr19:35944911, chr19:38907710, chr19:42211188, chr19:45712682, chr19:49464557, chr19:49881697, chr19:55284281, chr19:55615832, chr2:9003857, chr2:61166980, chr2:89848744, chr2:190440760, chr2:200981406, chr2:202250239, chr2:209772183, chr2:224690031, chr20:1185694, chr20:62122215, chr20:62309811, chr20:62328882, chr20:63667368, chr21:5137123, chr21:25735328, chr21:41879374, chr21:43882191, chr21:45277832, chr22:31098838, chr22:33058579, chr22:40026246, chr22:49884754, chr3:13366052, chr3:112523580, chr3:112523614, chr3:128428346, chr3:138798882, chr3:150408978, chr3:157067444, chr3:165348114, chr3:165348116, chr3:186783828, chr3:197297920, chr4:1229607, chr4:4438102, chr5:3750940, chr5:6615113, chr5:16179849, chr5:16179857, chr5:16179865, chr5:16179878, chr5:16179881, chr5:16179883, chr5:16180008, chr5:178204129, chr5:178204131, chr6:3577167, chr6:27279630, chr6:73310426, chr6:73310429, chr6:73310454, chr6:73310463, chr6:125791186, chr6:136290429, chr7:996193, chr7:1551166, chr7:1849861, chr7:1854236, chr7:4730195, chr7:5070131, chr7:6333296, chr7:15848453, chr7:26247657, chr7:29643153, chr7:30043812, chr7:43877706, chr7:44146254, chr7:44748739, chr7:149022546, chr8:31640044, chr8:31640053, chr8:31640067, chr8:42842942, chr8:80578027, chr9:107245787, chr9:120913537, chr9:126408197, chr9:131584010, chr9:136197462, chr9:136451036, chr9:136451222, chr9:136451519, chr9:137013461, chr9:137013470, chr9:137013473, chr9:137013486, chr9:137278360, chr9:137278384, chr9:137278402, chr9:137278488, chrX:150899022, chr6:34553001, chr12:118980854, chr19:811773, chr2:130345606, chr2:202250236, chr7:1005784, chr19:7934275, chr14:34024477, chr7:107390004, chr1:25216043, chr15:85610808, chr19:5668214, chr7:5297184, chr1:241356405, chr10:102714534, chr8:31640064, chr12:120967099, chr19:1393965, chr15:25438570, chr15:74789270, chr7:5297124, chr8:123443315, chr1:180269892, chr19:1456332, chr16:73028748, chr2:75056446, chr19:18543346, chr2:75056435, chr2:75056445, chr16:29876586, chr6:43771035, chr12:124344199, chr8:139649190, chr2:230068915, chr1:6461087, chr1:234661304, chr12:7824990, chr16:71931876, chr21:36736273, chr3:183636771, chr5:143405413, chr10:69402478, chr14:104887313, chr16:1315523, chr7:2295681, chr1:214108494, chr1:236064214, chr2:44168465, chr2:114662163, chr3:107999730, chr6:3577155, chr6:3577177, chr7:57818168, chr1:41324219, chr1:213051051, chr11:4521815, chr12:43552603, chr17:35488134, chr3:14401986, chr6:13450944, chr9:101738883, chr1:167221470, chr16:88880826, chr19:3652127, chr20:17396399, chr12:104599364, chr16:87827109, chr19:17873403, chr19:17873409, chr19:17873411, chr6:63656136, chr12:45216305, chr12:57837371, chr16:21557088, chr17:34331252, chr21:39054428, chr7:3910633, chr16:3655081, chr18:12324746, chr9:76701152, chr19:1648427, chr20:47468584, chr3:96841981, chr1:239386481, chr19:3088153, chr20:17396185, chr6:27557116, chr6:55680614, chr7:5297111, chr19:58440360, chr7:151111578, chr8:139649329, chr19:28520192, chr6:34778112, chr9:131112163, chr7:102201787, chr8:42153025, chr8:42622755, chr1:154174789, chr17:49816136, chr19:7934266, chr19:13207507, chr2:17317571, chr5:3754702, chr6:24626028, chr6:44653805, chr2:173307231, chr6:22509101, chr8:107889762, chr5:128879466, chr19:3094373, chr17:72235698, chr21:34510358, chr9:137013468, chr9:137013480, chr1:220001751, chr5:308909, chr1:155544582, chr1:204089544, chr11:107970310, chr12:95066688, chr19:16553992, chr5:16179995, chr5:16180003, chr5:25900907, chr8:35235230, chr16:28617820, chr17:76419894, chr19:48194427, chr7:14695759, chr1:6470978, chr1:23636028, chr20:350196, chr1:31193833, chr8:126862783, chr11:78046094, chr12:132528585, chr18:79375872, chr19:652099, chr21:9873175, chr7:57821358, chr7:101967581, chr8:144107705, chr12:62602516, chr1:6460819, chr1:244198034, chr19:5704451, chr9:137278356, chr10:76887333, chr19:1433766, chr19:13830675, chr20:37985816, chr1:15945048, chr14:23078131, chr9:133459357, chr8:31640070, chr12:62799867, chr5:32446706, chr12:120967103, chr5:128879583, chr15:69087040, chr20:347269, chr9:132325224, chr16:78195948, chr19:28824412, chr19:44753544, chr22:11288465, chr17:72123931, chr17:72124220, chr17:72124238, chr2:36973823, chr5:16179886, chr20:44560834, chr12:50057840, chr19:1456317, chr7:1421100, chr8:123443370, chr17:82626176, chr18:63283742, chr11:65650576, chr12:31591065, chr16:73028716, chr6:106135145, chr1:230399219, chr17:80599748, chr3:136614019, chr9:93826472, chr19:812407, chr6:135771171, chr9:137278500, chr16:3987194, chr17:49810078, chr2:164717792, chr22:40600900, chr3:123976907, chr3:123976934, chr2:89244905, chr19:5668194, chr11:78046073, chr9:127507984, chr9:120877473, chr1:159210159, chr6:44441882, chr8:101950035, chr1:11331701, chr19:1992998, chr19:1993003, chr9:128704978, chr9:124453638, chr19:1468243, chr16:2429260, chr20:62309824, chr7:44146237, chr8:31640050, chr9:94914976, chr12:37419025, chr12:45776142, chr17:42670166, chr19:1633551, chr9:137281710, chr1:214281447, chr6:34553086, chr1:167918494, chr17:72123590, chr19:45067160, chr6:157300486, chr7:1734991, chr7:1734996, chr1:70338695, chr19:18868002, chr9:14041343, chr10:76887324, chr19:7934351, chr19:13644740, chr2:5691280, chr5:134550960, chr12:95633568, chr1:6460956, chr16:17134583, chr16:17134595, chr16:61149835, chr19:4101376, chr19:37692212, chr6:1494847, chr7:38178231, chr7:71132295, chr11:134072686, chr16:2720277, chr16:11345580, chr17:29613358, chr18:14867181, chr19:3613344, chr2:209772188, chr6:34552991, chr6:88962788, chr12:105977085, chr17:7479367, chr17:21379559, chr19:10502839, chr19:39178093, chr21:43313606, chr22:31306646, chr3:11717633, chr3:139539693, chr7:4730228, and/or chr4:154375969, including any combination thereof.

In some embodiments, SCLC-I is identified based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 5. Analyses of each and every specific combination of the methylation sites of Table 5 are contemplated herein. It is specifically contemplated that any one or more of the methylation sites of Table 5 may be excluded in embodiments described herein. In some embodiments, SCLC-I is identified based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 methylation sites of Table 10. Analyses of each and every specific combination of the methylation sites of Table 10 are contemplated herein. It is specifically contemplated that any one or more of the methylation sites of Table 10 may be excluded in embodiments described herein. In some embodiments, SCLC-I is identified based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30 methylation sites of Table 18. Analyses of each and every specific combination of the methylation sites of Table 18 are contemplated herein. It is specifically contemplated that any one or more of the methylation sites of Table 18 may be excluded in embodiments described herein. In some embodiments, SCLC-I is identified based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, or 848 methylation sites of Table 23. Analyses of each and every specific combination of the methylation sites of Table 23 are contemplated herein. It is specifically contemplated that any one or more of the methylation sites of Table 23 may be excluded in embodiments described herein. In some embodiments, SCLC-I is identified based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 30. Analyses of each and every specific combination of the methylation sites of Table 30 are contemplated herein. It is specifically contemplated that any one or more of the methylation sites of Table 30 may be excluded in embodiments described herein. In some embodiments, SCLC-I is identified based on detection of differential methylation of cg09248054, cg02379560, cg04917391, cg05020685, cg06485940, cg16364495, cg05857126, cg13780782, cg02008691, cg24664798, cg17265693, cg06508056, cg15932065, cg16405211, cg18804920, cg23889772, cg07270851, cg24238564, cg03850035, cg02659920, cg05651265, cg10976861, cg24127874, cg02365303, cg11799006, cg00336977, cg02125259, cg09433131, cg14865862, cg20136513, cg00378510, cg06473097, cg23844705, cg19741167, cg16769791, cg00448761, cg04303901, cg13077545, cg15559684, cg07035875, cg19115272, cg06093379, cg19154027, and/or cg22073838. In some embodiments, SCLC-I is identified based on detection of differential methylation of chr1:1040462, chr1:1040475, chr1:1682754, chr1:6249894, chr1:6249902, chr1:6249914, chr1:6249917, chr1:12191832, chr1:15741578, chr1:16980570, chr1:16980579, chr1:16980593, chr1:18630506, chr1:20487144, chr1:21574155, chr1:23801501, chr1:24730777, chr1:24730787, chr1:24730788, chr1:24730796, chr1:24730797, chr1:25195867, chr1:31065986, chr1:36323440, chr1:39491967, chr1:43285575, chr1:43285577, chr1:43369421, chr1:89843439, chr1:89843473, chr1:145960916, chr1:153532886, chr1:153532890, chr1:156908050, chr1:163404039, chr1:163423088, chr1:163423133, chr1:163423188, chr1:163423194, chr1:163423199, chr1:163546298, chr1:164136579, chr1:164136587, chr1:164136632, chr1:164236230, chr1:164236242, chr1:165235591, chr1:167796909, chr1:208601823, chr1:213771516, chr1:213820223, chr1:213861854, chr1:213861907, chr1:213910457, chr1:225962005, chr1:230322908, chr1:240992226, chr1:241128149, chr1:243916475, chr10:99431109, chr10:132518774, chr11:366066, chr11:438565, chr11:438607, chr11:726201, chr11:848890, chr11:848923, chr11:848927, chr11:848933, chr11:849115, chr11:849137, chr11:849141, chr11:849142, chr11:849148, chr11:849149, chr11:17734970, chr11:44566012, chr11:45922046, chr11:45922074, chr11:45922076, chr11:45922090, chr11:45922100, chr11:45922275, chr11:45922276, chr11:45923059, chr11:45923077, chr11:46381467, chr11:64299743, chr11:64299761, chr11:64300844, chr11:64300851, chr11:64300858, chr11:64300884, chr11:64490631, chr11:64490632, chr11:64712787, chr11:65355635, chr11:65355646, chr11:65355670, chr11:65551424, chr11:65551425, chr11:67023317, chr11:67464932, chr11:67464964, chr11:73264689, chr11:75240135, chr11:85811258, chr11:89593934, chr11:92709117, chr11:93542959, chr11:114059499, chr11:114059530, chr11:114059757, chr11:114061199, chr11:114061210, chr11:114061212, chr11:114061215, chr11:114061239, chr11:114631735, chr11:115504986, chr11:115504997, chr11:115505001, chr11:117432347, chr11:117795858, chr11:118956922, chr11:119182275, chr11:119206066, chr11:119729116, chr11:120284546, chr11:124746873, chr11:124746909, chr11:124746913, chr11:124863373, chr11:124863394, chr11:125263544, chr11:129316134, chr11:130070945, chr11:130159700, chr11:130159829, chr11:130159851, chr11:130159854, chr11:133064200, chr11:133064212, chr11:133064221, chr11:133064231, chr11:133064233, chr11:134311382, chr12:6961918, chr12:6962633, chr12:6962646, chr12:6962648, chr12:6964182, chr12:16504002, chr12:16714352, chr12:16714353, chr12:25939377, chr12:32470696, chr12:32646853, chr12:42238116, chr12:49346692, chr12:51392037, chr12:52903995, chr12:52904021, chr12:52948914, chr12:96738497, chr12:96738507, chr12:97138576, chr12:97324958, chr12:97325039, chr12:97607417, chr12:97619683, chr12:97755215, chr12:97855295, chr12:97924591, chr12:98350733, chr12:103100480, chr12:103165897, chr12:103165901, chr12:103165909, chr12:103165933, chr12:108310791, chr12:108310792, chr12:108310807, chr12:109291976, chr12:109292004, chr12:119258242, chr12:119258280, chr12:132134549, chr12:132134577, chr12:132134595, chr12:132134596, chr12:132134600, chr12:132134601, chr12:132134630, chr13:21298314, chr13:34918433, chr13:35920681, chr13:38494047, chr13:38545892, chr13:39205350, chr13:39300854, chr13:42040772, chr13:54129564, chr13:54844155, chr13:55129970, chr13:55166399, chr13:59341409, chr13:70033781, chr13:73632816, chr13:73632851, chr13:78840351, chr13:101796812, chr13:101927978, chr13:101933364, chr13:102060417, chr13:102060419, chr13:102060451, chr13:102068532, chr13:102068551, chr13:102068563, chr13:102068572, chr13:102390033, chr13:110473172, chr13:110536576, chr13:110825816, chr13:110830973, chr13:112988667, chr13:113016746, chr13:113433650, chr14:21069139, chr14:21069177, chr14:21069183, chr14:22883379, chr14:26553939, chr14:29450905, chr14:29450950, chr14:29523990, chr14:30391455, chr14:37657127, chr14:37751915, chr14:37751924, chr14:37751973, chr14:37751985, chr14:37772760, chr14:37772778, chr14:37772795, chr14:37802591, chr14:38033650, chr14:38033658, chr14:38115498, chr14:38115499, chr14:38115532, chr14:38129918, chr14:39442503, chr14:44156407, chr14:44156411, chr14:44156431, chr14:44379746, chr14:44379797, chr14:53956630, chr14:53956642, chr14:53956653, chr14:53956667, chr14:56506990, chr14:56534824, chr14:56832505, chr14:56992482, chr14:56992483, chr14:61348113, chr14:62736524, chr14:63064707, chr14:63114304, chr14:63114311, chr14:63114337, chr14:64741044, chr14:64942593, chr14:64942636, chr14:67241487, chr14:88791865, chr14:88791879, chr14:104827290, chr14:104895812, chr14:104895822, chr15:22702324, chr15:43776557, chr15:43776576, chr15:53791638, chr16:64387, chr16:561080, chr16:561087, chr16:677534, chr16:2148616, chr16:4260566, chr16:14303673, chr16:14303701, chr16:14303702, chr16:14303720, chr16:19522006, chr16:52395111, chr16:52395146, chr16:52504927, chr16:52504937, chr16:52504992, chr16:52621872, chr16:56191818, chr16:58500253, chr16:58500281, chr16:58500286, chr16:67666791, chr16:67666821, chr16:67666825, chr16:67666999, chr16:67667017, chr16:67667021, chr16:71626053, chr16:84312964, chr16:84312972, chr16:84312978, chr16:84519935, chr16:84659693, chr16:84842521, chr16:85449391, chr16:85449403, chr16:85449420, chr16:86467633, chr16:88291496, chr16:88302508, chr16:88737568, chr17:4263552, chr17:4449947, chr17:7044006, chr17:17403801, chr17:17403831, chr17:19379152, chr17:19379174, chr17:19379191, chr17:29176890, chr17:29565877, chr17:29565896, chr17:29565920, chr17:31561710, chr17:41527810, chr17:42951031, chr17:43720600, chr17:48892861, chr17:65625809, chr17:68870337, chr17:74213330, chr17:74961013, chr17:74961036, chr17:74972136, chr17:79794767, chr17:81397114, chr17:81397126, chr17:81397150, chr17:81514240, chr17:81902419, chr17:81902429, chr17:81902430, chr17:81902433, chr17:81902434, chr17:81902440, chr17:81902441, chr18:6929505, chr18:7327489, chr18:7327495, chr18:32928012, chr18:32928017, chr18:32928020, chr18:32928024, chr18:33167723, chr18:33167727, chr18:33225321, chr18:33317997, chr18:33318065, chr18:33570344, chr18:34609374, chr18:34639003, chr18:38714972, chr18:38714994, chr18:43102815, chr18:47748194, chr18:59148758, chr18:59148765, chr18:63207225, chr18:63207233, chr18:63207260, chr18:71409912, chr18:71652314, chr18:71652318, chr18:72662276, chr18:72677583, chr18:76446564, chr18:76446573, chr18:76446574, chr18:76446612, chr18:76472706, chr18:76580424, chr18:76590712, chr18:76590755, chr18:76590771, chr18:77020807, chr18:77132839, chr18:79334799, chr19:460731, chr19:460763, chr19:511206, chr19:511219, chr19:537165, chr19:1082036, chr19:1082073, chr19:1082079, chr19:1083180, chr19:2624624, chr19:4147445, chr19:4566632, chr19:4566647, chr19:4955146, chr19:6464556, chr19:6551811, chr19:6583884, chr19:7115981, chr19:7904264, chr19:9953412, chr19:9953424, chr19:9961699, chr19:9966503, chr19:9966579, chr19:9966586, chr19:9997385, chr19:10625441, chr19:10625699, chr19:11418683, chr19:13099153, chr19:13248768, chr19:14433259, chr19:15195814, chr19:15195823, chr19:15195830, chr19:15195831, chr19:15195838, chr19:15195844, chr19:15195847, chr19:15195851, chr19:17041847, chr19:17251851, chr19:28293220, chr19:29892627, chr19:33260980, chr19:33393584, chr19:36008846, chr19:36009562, chr19:42242259, chr19:43757320, chr19:44796859, chr19:45444316, chr19:45598430, chr19:46016118, chr19:46016119, chr19:46016123, chr19:46016124, chr19:46016125, chr19:46016126, chr19:46016130, chr19:46016136, chr19:46016138, chr19:46016139, chr19:46016142, chr19:46016144, chr19:46016145, chr19:46016146, chr19:46016147, chr19:46016148, chr19:46016149, chr19:46016465, chr19:48752569, chr19:48752624, chr19:48752637, chr19:48753115, chr19:48753117, chr19:48753159, chr19:48753363, chr19:48753390, chr19:50417522, chr19:50418925, chr19:51030848, chr19:51077450, chr19:52028350, chr19:53991315, chr19:55374611, chr19:55554857, chr2:3303753, chr2:4107720, chr2:4798818, chr2:6987052, chr2:7032109, chr2:8457740, chr2:14507507, chr2:25190664, chr2:47369968.

In some embodiments, tumor DNA from a subject is further determined to have differential methylation at one or more methylation sites of Table 13. In some embodiments, the subject is further determined to have differential methylation at at least or at most 1, 2, 3, 4, 5, 6, 7, 8, 9, 10, 11, 12, 13, 14, 15, 16, 17, 18, 19, 20, 21, 22, 23, 24, 25, 26, or 27 methylation sites of Table 13. Analyses of each and every specific combination of the methylation sites of Table 13 are contemplated herein. It is specifically contemplated that any one or more of the methylation sites of Table 13 may be excluded in embodiments described herein. Determining tumor DNA from a subject to have differential methylation at one or more methylation sites of Table 13 may be useful in, for example, confirming that the subject has SCLC.

A treatment for the subject may be determined based on the subtype determination. Such treatment may also be in combination with another therapeutic regime, such as chemotherapy or immunotherapy. In addition, the treatment may be in combination due to a subject's cancer falling into more than one subtype, such as, for example, if one portion of the cancer cells fall into the SCLC-A subtype (e.g., express ASCL1 and/or comprise differential methylation at two or more methylation sites from Tables 2 and/or 7) and another portion of the cancer cells fall into the SCLC-N subtype (e.g., express NEUROD1 and/or comprise differential methylation at two or more methylation sites from Tables 3 and/or 8). The type and/or subtype of a given cancer may change over time, and in some embodiments the present methods regarding identifying the type and/or subtype and selecting an appropriate treatment are performed more than once, such as repeating the methods after a patient develops resistance to a selected therapy, or after a predetermined period of time, and modifying the therapy accordingly.

In some embodiments, a subject is or was determined to have a cancer of the SCLC-A subtype (e.g., determined to have differential methylation at two or more methylation sites from Tables 2, 7, 15, 20 and/or 27). In some embodiments, the subject is administered a B-cell lymphoma 2 (BCL-2) inhibitor. A BCL-2 inhibitor may describe any agent, molecule, or compound capable of inhibiting the activity of a BCL-2 family protein. Examples of BCL-2 inhibitors include ABT-737, ABT-263 (navitoclax), ABT-199 (venetoclax), GX15-070 (obatoclax), HA14-1, TW-37, AT101, and BI-97C1 (sabutoclax). In some embodiments, the BCL-2 inhibitor is ABT-737 or navitoclax. In some embodiments, the subject is administered a DLL3-targeted therapeutic. A DLL3-targeted therapeutic, as used herein, describes any agent, molecule, or compound capable of binding to a DLL3 protein and having therapeutic properties in treating cancer, including small cell lung cancer such as SCLC-A. In some embodiments, the DLL3-targeted therapeutic is an anti-DLL3 antibody or fragment thereof. In some embodiments, the DLL3-targeted therapeutic is rovalpituzumab. In some embodiments, the DLL3-targeted therapeutic is an antibody-drug conjugate. In some embodiments, the DLL3-targeted therapeutic is rovalpituzumab tesirine. In some embodiments, the DLL3-targeted therapeutic is a DLL3-targeted cellular therapy. DLL3-targeted cellular therapies include any cell-based therapeutic capable of binding to DLL3. A DLL3-targeted therapeutic may be an immune cell capable of targeting DLL3-expressing cells, for example, via expression of a DLL3-binding agent such as a DLL3-targeted chimeric antigen receptor (CAR) or T cell recept (TCR). In some embodiments, the DLL3-targeted cellular therapy is a DLL3-targeted CAR T cell. In some embodiments, the DLL3-targeted cellular therapy is a DLL3-targeted CAR NK cell.

In some embodiments, a subject is or was determined to have a cancer of the SCLC-N subtype (e.g., determined to have differential methylation at two or more methylation sites from Tables 3, 8, 16 21, and/or 28). In some embodiments, the subject is administered an Aurora kinase (AURK) inhibitor, a JAK inhibitor, or a c-Met inhibitor. In some embodiments, the subject is administered an AURK inhibitor. Examples of AURK inhibitors include alisertib, ZM447439, hesperidin, ilorasertib, VX-680, CCT 137690, lestaurtinib, NU 6140, PF 03814735, SNS 314 mesylate, TC-A 2317 hydrochloride, TAK-901, AMG-900, AS-703569, AT-9283, CYC-116, SCH-1473759, and TC-S 7010. In some embodiments, the AURK inhibitor is CYC-116, alisertib, or AS-703569. Examples of JAK inhibitors include ruxolitinib, tofacitinib, oclacitinib, baricitinib, peficitinib, fedratinib, upadacitinib, filgotinib, cerdulatinib, gandotinib, lestaurtinib, momelotinib, pacritinib, and PF-04975842. Examples of c-Met inhibitors include BMS-777607, cabozantinib, MK-2461, AMG-458, JNJ-38877605, PF-04217903, and GSK-1363089. Other drugs to which subjects having a cancer of the SCLC-N subtype may be sensitive include PF-562271, VS-507, KW-2449, pimozide, CB-64D, AC-220, omacetaxine mepasuccinate, XL-888, XL-880, ifosfamide, SL-0101, GW-5074, letrozole, CYC-202, and BIM-46187.

In some embodiments, a subject is or was determined to have a cancer of the SCLC-P subtype (e.g., determined to have differential methylation at two or more methylation sites from Tables 4, 9, 17, 22, and/or 29). In some embodiments, the subject is administered a PARP inhibitor, an AKT inhibitor, a Sky inhibitor, a JAK inhibitor, a SRC inhibitor, a BET inhibitor, an ERK inhibitor, an mTor inhibitor, an HSP90 inhibitor, a PI3K inhibitor, a CDK inhibitor, a topoisomerase inhibitor, a nucleoside analogue, an anti-metabolite, or a platinum-containing chemotherapeutic agent. Examples of PARP inhibitors include olaparib, rucaparib, niraparib, talazoparib, veliparib, pamiparib, CEP 9722, E7016, iniparib, AZD2461, and 3-aminobenzamide. In some embodiments, the PARP inhibitor is talazoparib, olaparib, niraparib, AZD-2461, or rucaparib. Examples of AKT inhibitors include CCT-128930, GSK690693, MK 2206, SC79, capivasertib, ipatasertib, borussertib, uprosertib, perifosine, AZD-5363, and A-443654. Examples of Syk inhibitors include R-406, R-788 (fostamatinib), BAY 61-3606, and nilvadipine. Examples of JAK inhibitors include ruxolitinib, tofacitinib, oclacitinib, baricitinib, peficitinib, fedratinib, upadacitinib, filgotinib, cerdulatinib, gandotinib, lestaurtinib, momelotinib, pacritinib, AZD-1480, XL-019, SB-1578, WL-1034, and PF-04975842. Examples of SRC inhibitors include dasatinib, AZD-0530, KX2-391, bosutinib, saracatinib, and quercetin. Examples of BET inhibitors include GSK1210151A, GSK525762, (+)-JQ1, OTX-015, TEN-010, CPI-203, CPI-0610, LY294002, AZD5153, MT-1, and MS645. Examples of ERK inhibitors include SC-1 (pluripotin), AX 15836, BIX 02189, ERK5-IN-1, FR 180204, TCS ERK 11e, TMCB, and XMD 8-92. Examples of CDK inhibitors include R-547, palbociclib, LY-2835219, CYC-202, ribociclib, abemaciclib, and trilaciclib. Examples of mTor inhibitors include PF-04212384, OSI-027, rapamycin, AZD-2014, RG-7603, BGT-226, PI-103, GSK-2126458, everolimus, temsirolimus, ridaforolimus, sirolimus, dactolisib, and sapanisertib. Examples of anti-metabolites and nucleoside analogues include teriflunomide, pemetrexed, ONX-0801, fluorouracil, cladribine, methotrexate, mercaptopurine, gemcitabine, capecitabine, hydroxyurea, fludarabine, 2-fluoroadenosine, pralatrexate, nelarabine, cladribine, clofarabine, decitabine, azacitidine, cytarabine, floxuridine, and thioguanine. In some embodiments, the anti-metabolite is pemetrexed, methotrexate, or pralatrexate. In some embodiments, the nucleoside analog is floxuridine, cytarabine, clofarabine, or fludarabine. Examples of platinum-containing chemotherapeutic agents include cisplatin, carboplatin, oxaliplatin, nedaplatin, picoplatin, and satraplatin. In some embodiments, the platinum-containing chemotherapeutic agent is cisplatin, carboplatin, oxaliplatin, nedaplatin, picoplatin, or satraplatin. Other drugs to which patients having a cancer of the SCLC-P subtype may be sensitive include ENMD-2076, HPI-1, CP-868596, TL-32711, FGF inhibitor, AS-703569, vandetanib, CYC-116, KW-2499, GSK-2334470, BMS-582664, AEG-40730, ICG-001, CB-64D, SCH-1473759, MK-2461, CH-5132799, dovitinib, AM-2282, PP-242, ZSTK-474, crizotinib, apitolisib, AT-9283, MPC-3100, alisertib, LOR-253, INK-128, AZD-8055, omacetaxine mepasuccinate, everolimus, XL-888, XL-880, PF-04929113, PF-4942847, dactolisib, PF-04691502, TAK-901, CUDC-305, tretinoin, GSK-461364, BAY-80-6946, danorubicin, doxorubicin, valrubicin, YK-4-279, PF-4176340, BKM-120, APO-866, EB-1627, axitinib, XR-5944, XR-5000, BX-912, mitoxantrone, LY-294002, ixabepilone, GDC-0941, BMS-536924, 3-AP, thiotepa, belinostat, and ABT-348.

In some embodiments, a subject is or was determined to have a cancer of the SCLC-I subtype (e.g., determined to have differential methylation at two or more methylation sites from Tables 5, 10, 18, 23 and/or 30). These cells may express immune checkpoint proteins, inflammatory markers, STING pathway proteins, CCL5, CXCL10, MHC proteins, CD274 (PD-L1), LAG3, C10orf54 (VISTA), IDO1, CD38, and ICOS. In this case, the patient is selected for treatment with an immune checkpoint inhibitor, a BTK inhibitor, a Syk inhibitor, a multikinase inhibitor, an ERK inhibitor, an VEGFR inhibitor, a MEK inhibitor, and/or a FGFR inhibitor. Examples of BTK inhibitors include ibrutinib, LCB 03-0110, LFM-A13, PCI 29732, PF 06465469, and terreic acid. Examples of Syk inhibitors include R-406, R-788 (fostamatinib), BAY 61-3606, and nilvadipine. Examples of multikinase inhibitors include LY-2801653, ENMD-2076, ponatinib, and pazopanib. Examples of ERK inhibitors include SC-1 (pluripotin), AX 15836, BIX 02189, ERK5-IN-1, FR 180204, TCS ERK Ile, TMCB, and XMD 8-92. Examples of VEGFR inhibitors include ASP-4130 (tivozanib), lenvatinib, RG-7167, sorafenib, sunitinib, bevacizumab, cabozantinib, regorafenib, nintedanib, and apatinib. Examples of MEK inhibitors include RO-5126766, AZD-8330, TAK-733, XL-518, PD-0325901, ARRY-162, trametinib, pimasertib, cobimetinib, binimetinib, and selumetinib. Examples of FGFR inhibitors include AZD-4547, PD-173074, LY-2874455, BGJ-398, ponatinib, nintedanib, dovitinib, danusertib, and brivanib. Other drugs to which patients having a cancer of the SCLC-I subtype may be sensitive include AZD-1480, AZD-0530, ASP-3026, fulvestrant, SCH-1473759, MK-2461, LY-2090314, PP-242, 17-AAG, BPR1J-097, INK-128, AZD-8055, omacetaxine mepasuccinate, everolimus, XL-888. XL-880, dactolisib, PF-04691502. OSI-027, rapamycin, CUDC-305, and bleomycin.

### II. Classification

Aspects of the present disclosure are directed to methods for classification of a subject as having a small cell lung cancer (SCLC) of one of four subtypes: SCLC-A, SCLC-N, SCLC-P, or SCLC-I. In some embodiments, the subject is classified as having SCLC-A, SCLC-N, SCLC-P, or SCLC-I based, at least in part, on determining a methylation status of two or more methylation sites of Tables 1, 2, 3, 4, 5, 6, 7, 8, 9, 10, 15, 16, 17, 18, 19, 20, 21, 22, 23, 24, 26, 27, 28, 29, and/or 30. In some embodiments, the subject is classified as having SCLC-A, SCLC-N, SCLC-P, or SCLC-I based on determining a methylation status of at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 1, Table 20, Table 21, Table 22, Table 23, Table 27, Table 28, Table 29, or Table 30.

In some embodiments, a subject is classified as having SCLC-A. In some embodiments, a subject is classified as having SCLC-A based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 2. Analyses of each and every specific combination of the methylation sites of Table 2 are contemplated herein. In some embodiments, a subject is classified as having SCLC-A based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, or 30 methylation sites of Table 7. Analyses of each and every specific combination of the methylation sites of Table 7 are contemplated herein. In some embodiments, a subject is classified as having SCLC-A based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, or 30 methylation sites of Table 15. Analyses of each and every specific combination of the methylation sites of Table 15 are contemplated herein. In some embodiments, a subject is classified as having SCLC-A based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, 1000, 1100, 1200, 1300, or 1340 methylation sites of Table 20. Analyses of each and every specific combination of the methylation sites of Table 20 are contemplated herein. In some embodiments, SCLC-A is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 27. Analyses of each and every specific combination of the methylation sites of Table 27 are contemplated herein. It is specifically contemplated that any one or more of the methylation sites of Table 27 may be excluded in embodiments described herein. In some embodiments, SCLC-A is identified based on detection of differential methylation of cg00799539, cg04610718, cg10672201, cg17277939, cg11201256, cg07639982, cg01566028, cg06043710, cg01154505, cg09643186, cg03817675, cg00090674, cg02639667, cg22053861, cg14338051, cg00794178, cg16860004, cg04317756, cg01942646, cg11249931, cg08279731, cg05161074, cg00773370, cg07444408, cg08758185, cg27119612, cg22904437, cg04506569, cg04877165, and/or cg01610165. In some embodiments, SCLC-A is identified based on detection of differential methylation of chr17:74961036, chr17:74961013, chr18:59062159, chr19:13506705, chr9:134815629, chr21:41180000, chr9:93014411, chr9:114211349, chr9:134810271, chr19:3385734, chr6:51213948, chr18:27786347, chr6:168638645, chr9:134815657, chr9:134815611, chr16:84519934, chr20:20364625, chr5:172103442, chr20:22598951, chr16:85355101, chr9:134815628, chr20:20364629, chr19:511206, chr6:168149181, chr7:100122306, chr16:84519930, chr8:58989473, chr18:27786294, chr20:22557408, chr9:128177708, chr7:157633760, chr6:40349351, chr6:157229362, chr13:84927994, chr4:8101279, chr22:41667328, chr5:176843804, chr5:176529007, chr4:137426451, chr6:40349318, chr11:11578650, chr20:20364628, chr6:40349319, chr17:48190199, chr7:100122301, chr1:226589216, chr22:40022901, chr6:37078047, chr9:134765725, chr20:20364641, chr18:77020770, chr17:3695881, chr19:38253123, chr9:114255802, chr7:157017749, chr17:74823356, chr9:117355642, chr6:51213976, chr7:151045548, chr16:4537495, chr2:45274755, chr8:139674518, chr12:132134577, chr9:134885740, chr16:4537572, chr17:1961091, chr6:51213977, chr11:888785, chr9:134815626, chr13:110011941, chr19:13372294, chr16:84519955, chr20:20364626, chr20:20364640, chr17:57874806, chr6:168638634, chr7:14372215, chr22:50010360, chr14:56814573, chr13:112641259, chr7:146633722, chr7:157633743, chr21:43758530, chr6:168538915, chr5:176843791, chr2:46983311, chr2:239300749, chr7:157998148, chr6:34128215, chr13:112106712, chr16:3999700, chr9:114172063, chr9:134792142, chr11:11578688, chr7:157833048, chr1:151346323, chr21:38661697, chr1:226588954, chr20:6232329, chr9:134792130, chr9:134785070, chr9:134815627, chr9:134592825, chr16:84519980, chr7:100122290, chr11:11973687, chr19:38253193, chr14:44405368, chr16:4260161, chr2:28411073, chr1:50214861, chr16:84519943, chr22:19933408, chr11:68833022, chr7:73985377, chr9:93014375, chr9:114183920, chr16:4260632, chr9:133558991, chr6:168638701, chr7:157658598, chr19:13506729, chr19:13483181, chr16:84519942, chr11:76784947, chr11:65551424, chr7:2193699, chr5:179258981, chr14:99518884, chr18:27786301, chr11:64056580, chr6:169251928, chr9:134486435, chr12:132134595, chr16:84519935, chr21:41179996, chr14:105392911, chr7:40330569, chr13:111622152, chr2:98823202, chr17:29972557, chr9:134815918, chr1:213905614, chr7:2607131, chr4:8101234, chr12:103012503, chr14:44262090, chr6:82363058, chr9:114170339, chr6:168138608, chr12:11856447, chr19:13634667, chr9:93722421, chr9:114172123, chr17:29565848, chr11:64353390, chr9:90908530, chr16:3999757, chr11:65551425, chr9:114306707, chr19:2354863, chr9:136289024, chr6:107633714, chr7:157017791, chr6:168496984, chr3:45819148, chr18:77102188, chr12:105784446, chr17:29972550, chr11:119702010, chr5:1316124, chr7:73966925, chr3:53702765, chr11:118924054, chr19:51077543, chr17:81246338, chr12:3077928, chr12:130460997, chr7:157963798, chr12:130483815, chr7:157963771, chr14:64942593, chr18:59203056, chr19:13463517, chr20:20364646, chr7:157987469, chr2:216380208, chr20:16806905, chr17:74961041, chr3:176190636, chr20:22652824, chr6:37078083, chr14:44405319, chr18:72677631, chr9:136686592, chr2:216986073, chr2:10040203, chr21:38636406, chr9:134824994, chr12:119158214, chr7:5795946, chr22:19933350, chr9:134785048, chr13:111559772, chr16:88640994, chr17:29972549, chr6:168130574, chr6:168130602, chr9:114183394, chr9:120326115, chr3:177516507, chr7:110084403, chr22:50010361, chr18:76681573, chr7:158485004, chr21:38661630, chr15:53659351, chr19:13347369, chr19:39163809, chr17:48620272, chr18:71652249, chr6:168667391, chr10:133309889, chr1:241210311, chr19:46770637, chr20:22556743, chr2:47227055, chr7:150974698, chr20:22653042, chr14:73228468, chr19:18422970, chr21:40144935, chr16:85355024, chr19:13575256, chr9:134690692, chr6:82363052, chr7:122307220, chr7:157633801, chr1:43439683, chr16:64387, chr2:216986079, chr9:134885778, chr4:128846433, chr1:43550363, chr3:9180988, chr19:511219, chr20:62711715, chr11:47327260, chr6:51214001, chr5:10652237, chr4:6944534, chr21:38599607, chr6:168440545, chr1:18794157, chr8:52150618, chr9:133662377, chr16:4537619, chr19:39307494, chr19:55254005, chr12:130535047, chr5:82804, chr6:34128233, chr12:11856451, chr19:13506452, chr17:77452817, chr6:109290309, chr21:38661664, chr10:133309857, chr21:34667592, chr7:100477920, chr20:38294975, chr21:41648785, chr21:43758525, chr9:134822835, chr16:3728390, chr16:29604945, chr1:41727346, chr21:45032479, chr1:39954061, chr17:29972554, chr6:151809197, chr12:111287675, chr19:13463510, chr8:52150574, chr19:13014442, chr4:8164496, chr7:8417325, chr7:157673872, chr2:107461969, chr9:36781051, chr4:138912435, chr7:157594362, chr2:237733847, chr20:22652808, chr14:64942648, chr16:4260588, chr7:157603987, chr7:157540317, chr13:111515678, chr17:67488327, chr7:73990185, chr10:43849791, chr17:1961149, chr6:82585382, chr1:224184849, chr2:182053988, chr21:41508507, chr15:85942771, chr10:78139388, chr9:126420967, chr15:67806290, chr7:157963797, chr9:113593968, chr7:157875450, chr7:157875495, chr14:64942637, chr19:55254025, chr1:205447752, chr16:84519956, chr9:134797123, chr18:63207225, chr9:134772536, chr8:38495018, chr13:112107447, chr5:100864671, chr14:91418510, chr9:134768753, chr19:13245332, chr11:47327256, chr8:42275934, chr12:103165897, chr18:38714972, chr7:157824560, chr14:75978696, chr15:40873466, chr4:104663323, chr5:1316150, chr4:189096959, chr9:134750430, chr13:98405890, chr9:134825870, chr16:52621839, chr17:62653135, chr9:134881575, chr7:157633794, chr9:114170237, chr18:77020828, chr2:216840164, chr2:181684626, chr9:134819274, chr21:34743983, chr17:57874702, chr22:28923959, chr21:44698714, chr11:65551324, chr9:113593972, chr7:2193759, chr6:168440498, chr8:141264221, chr16:4260115, chr16:4537502, chr20:20365491, chr1:7491237, chr20:20364647, chr11:31893506, chr19:41704632, chr20:17068564, chr5:6543207, chr19:41323849, chr2:85733778, chr7:157685012, chr11:75240135, chr6:168315218, chr14:64633473, chr11:20056293, chr14:44379797, chr16:4537618, chr7:157864088, chr10:133309864, chr14:88256419, chr16:19425106, chr6:168355227, chr9:134765734, chr6:168453036, chr21:41490949, chr7:73989381, chr12:3077894, chr1:53462420, chr9:113593928, chr10:133309740, chr7:101666174, chr11:14974476, chr5:100944956, chr20:22557482, chr18:74782855, chr19:3656386, chr7:26397761, chr19:13463491, chr22:38087144, chr9:130535984, chr9:134892203, chr13:107429178, chr19:38253194, chr17:82078952, chr1:1174126, chr1:232307467, chr6:14499964, chr16:4260566, chr16:85286422, chr17:74006568, chr7:101707772, chr6:168496991, chr18:27786339, chr13:113793557, chr19:47209566, chr2:113325550, chr5:176529022, chr8:52150549, chr9:113594013, chr9:134490841, chr7:1620862, chr9:134179005, chr15:63053313, chr12:124617614, chr21:45032480, chr13:113723025, chr7:150945169, chr12:6961918, chr19:39307859, chr16:89948104, chr19:8153491, chr20:20365487, chr20:38294974, chr3:73790643, chr6:153237290, chr6:168130594, chr7:2524564, chr9:134785301, chr9:136367817, chr19:38253176, chr19:406561, chr1:52633297, chr9:93819344, chr11:76784948, chr14:36524358, chr13:112106739, chr6:157228665, chr7:157953525, chr10:101780858, chr12:110942244, chr6:43578006, chr2:51027486, chr7:157685871, chr21:45032448, chr21:45032452, chr7:157017829, chr11:65551413, chr7:157544388, chr6:50845964, chr5:138650055, chr1:229147368, chr9:134782536, chr9:134316337, chr12:124259764, chr11:11578685, chr20:17068489, chr20:20365493, chr22:28923940, chr22:28923966, chr1:153536136, chr9:93861253, chr11:64540473, chr2:229672283, chr7:157633759, chr9:134534796, chr9:114170276, chr19:5950505, chr1:32885121, chr12:6962651, chr18:27786338, chr18:63207261, chr19:41704681, chr8:141289585, chr11:124895535, chr17:57874844, chr11:75335191, chr18:71337086, chr2:46983412, chr1:212589163, chr20:48751499, chr4:78056611, chr3:176814702, chr17:81246299, chr2:3940818, chr12:98238503, chr9:114170300, chr11:924647, chr19:55254024, chr2:208703401, and/or chr20:22598941, including any combination thereof.

In some embodiments, a subject is classified as having SCLC-N. In some embodiments, a subject is classified as having SCLC-N based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 3. Analyses of each and every specific combination of the methylation sites of Table 3 are contemplated herein. In some embodiments, a subject is classified as having SCLC-N based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 methylation sites of Table 8. Analyses of each and every specific combination of the methylation sites of Table 8 are contemplated herein. In some embodiments, a subject is classified as having SCLC-A based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, or 30 methylation sites of Table 16. Analyses of each and every specific combination of the methylation sites of Table 16 are contemplated herein. In some embodiments, a subject is classified as having SCLC-N based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 650, or 695 methylation sites of Table 21. Analyses of each and every specific combination of the methylation sites of Table 21 are contemplated herein. In some embodiments, SCLC-N is identified based on detection of differential methylation at at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 28. It is specifically contemplated that any one or more of the methylation sites of Table 28 may be excluded in embodiments described herein. In some embodiments, SCLC-N is identified based on detection of differential methylation of cg20505457, cg04220881, cg14755690, cg19798702, cg03920522, cg12187160, cg02531277, cg18780243, cg19028997, cg02314596, cg16498113, cg19513004, cg17401435, cg25043538, cg00648184, cg10414350, cg09309024, cg06440275, cg01431993, cg11528849, cg16640855, cg00369376, cg02836529, cg20806345, cg22976218, cg13538006, cg11887270, cg23731742, cg02622825, cg00284708, cg05234415, cg24657047, cg23994112, cg08399017, cg14647703, cg13969327, cg05214130, cg08573199, cg09621610, cg13417891, cg02902261, cg00678971, cg02243624, cg13808641, cg15076368, and/or cg11964216. In some embodiments, SCLC-N is identified based on detection of differential methylation of chr1:54356529, chr1:109296245, chr1:109296247, chr1:220653171, chr1:220653195, chr11:35853956, chr11:64154503, chr11:64154571, chr11:64154574, chr12:108792675, chr17:72440313, chr17:72440361, chr2:223901322, chr20:19919227, chr3:52468694, chr5:160245925, chr6:44261803, chr8:42275934, chr9:131721310, chr9:131721341, chr9:131721355, chr9:131721389, chr2:216909627, chr20:19919173, chr9:131721356, chr22:50276667, chr17:72440360, chr6:37537738, chr1:220653165, chr1:220653180, chr8:140270696, chr12:108792684, chr19:3656274, chr1:54356527, chr19:47650943, chr7:149261799, chr2:216380208, chr17:72440372, chr9:131721340, chr9:131721342, chr2:216380274, chr11:67493309, chr2:65583301, chr5:160245988, chr3:52468622, chr17:74823299, chr1:160362850, chr12:31009628, chr14:64633473, chr18:59049918, chr20:63878908, chr5:16538225, chr6:19719869, chr6:34128215, chr6:34128233, chr1:109296235, chr3:52522225, chr8:134941130, chr7:149261769, chr19:3656352, chr2:85733778, chr4:850796, chr3:126013960, chr19:3655899, chr22:44049853, chr9:120242580, chr1:41657833, chr1:220653181, chr11:8239208, chr2:65583300, chr5:16538207, chr6:34128222, chr9:131721343, chr7:149261778, chr14:105178678, chr12:31009602, chr1:28869236, chr1:220653223, chr7:44254153, chr6:43475701, chr15:68347588, chr20:49345755, chr5:160245950, chr19:3656298, chr19:3655920, chr20:49345760, chr6:34128216, chr3:52468649, chr1:944700, chr1:54356503, chr1:54356526, chr1:54356528, chr2:216909691, chr5:160245949, chr21:41490949, chr17:50076230, chr2:25702014, chr19:3655770, chr9:120242557, chr16:85439239, chr1:53320062, chr5:16538206, chr6:19719865, chr11:855989, chr19:3655921, chr6:107633786, chr19:14182660, chr19:48350598, chr3:52468650, chr16:10622418, chr3:52468678, chr11:20055879, chr2:216909621, chr6:34128193, chr16:85287295, chr1:53320061, chr7:100890535, chr12:48675723, chr2:25701951, chr20:63835624, chr4:76612797, chr6:43475769, chr3:16132696, chr11:66866493, chr12:31009572, chr1:226588954, chr16:66960251, chr19:3656369, chr17:4809230, chr7:157018053, chr20:11271495, chr17:82001560, chr17:82001559, chr2:85770228, chr20:49345801, chr21:41458778, chr6:34128249, chr11:44963230, chr1:944730, chr12:108792716, chr22:44049787, chr3:52522201, chr4:6308154, chr2:85733793, chr19:12953781, chr1:43550353, chr21:41490965, chr12:108792685, chr3:16132702, chr1:15723882, chr1:54356555, chr12:130484433, chr17:4809161, chr6:19719861, chr6:26385134, chr6:168130602, chr19:13913675, chr16:85287080, chr11:888785, chr19:3655898, chr1:1921168, chr2:200087052, chr19:2354832, chr11:129699789, chr1:28869229, chr19:18422970, chr1:944737, chr22:44049781, chr20:25239829, chr9:120242569, chr6:148509695, chr1:153536157, chr9:120242617, chr12:31009603, chr18:62333487, chr8:140270737, chr19:3655888, chr17:82001529, chr7:102424329, chr2:200087071, chr16:85287095, chr11:855972, chr11:855981, chr9:120242622, chr19:3656250, chr20:19836028, chr1:944699, chr17:2418940, chr3:185397845, chr7:44151120, chr11:35853892, chr12:123253744, chr20:49345788, chr6:34128234, chr7:750747, chr2:205764439, chr18:59050139, chr5:172103442, chr9:120242544, chr1:28869207, chr16:11997774, chr8:134499858, chr17:29158240, chr1:54356559, chr6:34545466, chr8:134941060, chr10:101780858, chr19:12953855, chr3:53702770, chr12:56997304, chr6:34128223, chr18:77053845, chr20:22557408, chr6:110414306, chr9:136013178, chr1:230759612, chr6:44261814, chr6:42963345, chr9:95738191, chr16:85355101, chr19:2354863, chr1:53320016, chr16:70318894, chr16:85696894, chr17:82001584, chr2:200087032, chr7:128075308, chr1:32885121, chr21:41665312, chr7:128881062, chr6:169160984, chr3:196992182, chr6:40349319, chr7:44151126, chr1:54356214, chr7:5276684, chr19:38253104, chr1:204921255, chr1:224184849, chr11:48025532, chr5:16538197, chr1:153536113, chr1:153536123, chr9:124820965, chr21:41639350, chr9:113901422, chr1:11965505, chr6:168638645, chr17:29582881, chr3:71074388, chr9:134486435, chr19:48350605, chr19:3656178, chr11:118901571, chr1:43550383, chr12:123253763, chr16:30030208, chr21:41648785, chr9:91810447, chr10:79313956, chr6:169190341, chr16:85436880, chr1:1921165, chr1:205447760, chr1:153536136, chr1:153536146, chr11:19730746, chr3:141093639, chr2:159228539, chr21:17508899, chr11:66866541, chr17:66830069, chr20:49345770, chr20:63835571, chr9:127200650, chr19:4055918, chr9:131810494, chr19:38253123, chr1:60903781, chr22:20014786, chr1:25771857, chr17:67652635, chr9:93166951, chr13:73218843, chr7:136045406, chr14:73228468, chr17:50831246, chr7:30171980, chr17:67652633, chr1:28869065, chr13:113818528, chr2:200087031, chr2:200087051, chr6:168517547, chr6:35534674, chr6:35534714, chr6:148509713, chr19:3656386, chr11:19832444, chr13:112775959, chr22:19632813, chr5:760546, chr19:3950318, chr1:60903756, chr7:8070969, chr16:70730157, chr20:49345761, chr3:45819148, chr6:19719834, chr6:34128254, chr6:34128257, chr6:168130634, chr1:15723893, chr16:85287162, chr7:1815269, chr9:93166950, chr16:85287116, chr17:67652568, chr12:123253794, chr16:85287098, chr18:77077978, chr19:46714207, chr3:53702765, chr7:3998211, chr9:92729620, chr16:85436709, chr11:66866538, chr22:44248796, chr6:34545471, chr7:102424320, chr17:602149, chr20:22557482, chr6:3444679, chr17:57707866, chr16:11654442, chr19:3655812, chr13:109334658, chr1:43550373, chr2:73080399, chr9:131810462, chr1:227920141, chr1:153536121, chr20:50035301, chr20:38700627, chr21:41553133, chr21:41648778, chr6:168130595, chr6:168539582, chr7:5276649, chr21:40144935, chr19:3656261, chr3:139718818, chr1:10625828, chr3:185397835, chr7:110084403, chr5:176843791, chr6:40349351, chr9:113901566, chr21:41179996, chr4:528986, chr12:48675786, chr19:13634667, chr6:168130574, chr19:3656190, chr1:202168439, chr12:124428932, chr6:34545461, chr7:3998240, chr3:127304410, chr16:89847327, chr1:226638169, chr1:181128695, chr1:54356181, chr12:123253764, chr11:64056580, chr3:47236186, chr16:85436855, chr11:888597, chr19:4010290, chr1:53320091, chr1:205257276, chr19:4010298, chr21:41648770, chr7:122306890, chr20:11271554, chr4:7911302, chr1:28869195, chr16:2455154, chr5:176843804, chr7:5276677, chr1:220653196, chr11:888641, chr19:38253076, chr4:1858204, chr8:38495138, chr3:141093636, chr1:31627824, chr12:48675768, chr2:190034714, chr6:168638690, chr19:3656351, chr12:131864061, chr9:89591607, chr17:74823356, chr16:85287017, chr16:88887476, chr2:96174325, chr6:3444622, chr1:5917479, chr1:1240684, chr12:130483799, chr13:110123705, chr6:168496991, chr8:53733503, chr7:105689129, chr2:113974997, chr12:591924, chr15:65333873, chr3:194305584, chr19:4059381, chr18:59049874, chr1:224184822, chr11:76175216, chr6:168130594, chr19:3655954, chr19:34999970, chr6:26614205, chr10:78139388, chr11:122984321, chr19:3656172, chr1:43550344, chr12:129694731, chr19:3655828, chr7:151045548, chr12:130483815, chr5:78519838, chr11:888359, chr1:205032318, chr9:120242579, chr1:16486605, chr1:20348597, chr2:43203382, chr6:168130603, chr17:3695871, chr11:19714522, chr16:85439179, chr7:122306891, chr9:130233164, chr2:47020392, chr19:3655938, chr8:58989422, chr17:80001489, chr2:5530198, chr19:3656393, chr7:1815299, chr21:43860194, chr6:168355227, chr1:20348596, chr7:5478011, chr2:200087040, chr19:48371166, chr16:22288921, chr19:12953833, chr11:888360, chr5:16538196, chr1:9251757, chr11:924646, chr12:131864094, chr2:25701937, chr6:168538915, chr9:127636823, chr1:22809032, chr17:29065994, chr6:168517537, chr4:6308178, chr1:61198659, chr20:11271453, chr7:44151141, chr19:18745096, chr7:30172111, chr6:34433316, chr12:119869919, chr13:110123752, chr17:29582888, chr18:36468445, chr19:4010297, chr19:39307494, chr20:63835620, chr21:41375919, chr5:6859175, chr6:37537680, chr7:100122236, chr9:93959419, chr9:91810580, chr17:73726103, chr1:226589216, chr9:23452269, chr6:42963307, chr8:140321186, and/or chr16:85287074, including any combination thereof.

In some embodiments, a subject is classified as having SCLC-P. In some embodiments, a subject is classified as having SCLC-P based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 4. Analyses of each and every specific combination of the methylation sites of Table 4 are contemplated herein. In some embodiments, a subject is classified as having SCLC-P based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, or 93 methylation sites of Table 9. Analyses of each and every specific combination of the methylation sites of Table 9 are contemplated herein. In some embodiments, a subject is classified as having SCLC-A based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, or 30 methylation sites of Table 17. Analyses of each and every specific combination of the methylation sites of Table 17 are contemplated herein. In some embodiments, a subject is classified as having SCLC-P based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 989 methylation sites of Table 22. Analyses of each and every specific combination of the methylation sites of Table 22 are contemplated herein. In some embodiments, SCLC-P is identified based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 29. Analyses of each and every specific combination of the methylation sites of Table 29 are contemplated herein. It is specifically contemplated that any one or more of the methylation sites of Table 29 may be excluded in embodiments described herein. In some embodiments, SCLC-P is identified based on detection of differential methylation of cg27232389, cg03020852, cg11691710, cg14197123, cg17885507, cg03517570, cg13297671, cg18795320, cg03075214, cg06832246, cg05683632, cg00704369, cg22171098, cg09934399, cg02817764, cg24677222, cg16770048, cg14701925, cg04182076, cg18969798, cg03803789, cg13240089, cg17758792, cg06923861, cg07956003, cg08275278, cg04983681, cg17728697, cg09472222, cg02220284, cg05366160, cg13705753, cg06839854, cg02675973, cg05395645, cg25601286, cg11701604, cg07825782, cg00600617, cg16054907, cg01489441, cg15564226, cg10026427, cg01149239, cg04479757, cg11019743, cg13207778, cg25296465, cg03387092, cg14175330, cg05999426, cg19011089, cg14313328, cg18529415, cg04563108, cg25874150, cg16318349, cg13081156, cg14088354, cg03264729, cg01580044, cg07313835, cg11597418, cg26855672, cg03356778, cg05216984, cg02593168, cg20314331, cg26797073, cg15873449, cg18983310, cg08265811, cg22340762, cg00618183, cg12452539, cg15786168, cg06669752, cg24136932, cg24272324, cg04619304, cg01772824, cg13921012, cg06542565, cg10288437, cg10609655, cg07262682, cg21289124, cg07325342, cg14211075, cg00719067, cg05554594, cg08368885, and/or cg21876001. In some embodiments, SCLC-P is identified based on detection of differential methylation of chr1:11032833, chr1:15207848, chr1:15945017, chr1:17026179, chr1:25741499, chr1:27551002, chr1:32327585, chr1:42682988, chr1:52365896, chr1:56552275, chr1:116407596, chr1:147829076, chr1:156538486, chr1:156750879, chr1:164576909, chr1:197915932, chr1:204289083, chr1:204571233, chr1:212618744, chr1:212618752, chr1:212638013, chr1:231162869, chr1:231162872, chr1:231162880, chr10:14365352, chr10:55631088, chr10:55631097, chr10:97713353, chr10:97713362, chr10:123017613, chr11:63997490, chr11:65536690, chr11:75410397, chr11:77750252, chr11:126313324, chr12:433809, chr12:7291128, chr12:57128650, chr12:67389978, chr12:75861004, chr12:81759542, chr12:85489437, chr12:101309330, chr12:103993054, chr12:104599362, chr12:104599370, chr12:105977083, chr12:110245057, chr12:110245058, chr12:110245093, chr12:111841988, chr12:120456258, chr12:122032753, chr12:122032775, chr12:124497504, chr12:124524949, chr12:124631301, chr13:20345144, chr14:89701670, chr14:105392906, chr14:105392911, chr15:43746172, chr15:58701617, chr15:74789232, chr15:74789276, chr15:74789281, chr16:234790, chr16:234826, chr16:648840, chr16:1498961, chr16:1746192, chr16:2075993, chr16:2964040, chr16:4767447, chr16:8621572, chr16:28193303, chr16:28863649, chr16:29906931, chr16:30125736, chr16:30973692, chr16:66696829, chr16:68341637, chr16:85611879, chr17:21665928, chr17:29576080, chr17:34252590, chr17:43187274, chr17:48932449, chr17:49816143, chr17:63383824, chr17:63383830, chr17:63739476, chr17:64661931, chr17:68205724, chr17:72165644, chr17:76420086, chr17:77439476, chr17:77439513, chr17:81248481, chr17:81248517, chr18:5543758, chr18:5543760, chr18:5543767, chr18:5543774, chr18:14907472, chr18:28175723, chr18:28175730, chr18:28175734, chr18:28175743, chr18:57574427, chr19:807444, chr19:811705, chr19:991153, chr19:991161, chr19:1633524, chr19:1993008, chr19:2115209, chr19:5618219, chr19:11483864, chr19:11483872, chr19:11483877, chr19:11483882, chr19:11483887, chr19:11483902, chr19:11483907, chr19:11483912, chr19:14206288, chr19:14559510, chr19:16628375, chr19:17209718, chr19:17209725, chr19:17209733, chr19:17209752, chr19:18778224, chr19:35721194, chr19:35944911, chr19:38907710, chr19:42211188, chr19:45712682, chr19:49464557, chr19:49881697, chr19:55284281, chr19:55615832, chr2:9003857, chr2:61166980, chr2:89848744, chr2:190440760, chr2:200981406, chr2:202250239, chr2:209772183, chr2:224690031, chr20:1185694, chr20:62122215, chr20:62309811, chr20:62328882, chr20:63667368, chr21:5137123, chr21:25735328, chr21:41879374, chr21:43882191, chr21:45277832, chr22:31098838, chr22:33058579, chr22:40026246, chr22:49884754, chr3:13366052, chr3:112523580, chr3:112523614, chr3:128428346, chr3:138798882, chr3:150408978, chr3:157067444, chr3:165348114, chr3:165348116, chr3:186783828, chr3:197297920, chr4:1229607, chr4:4438102, chr5:3750940, chr5:6615113, chr5:16179849, chr5:16179857, chr5:16179865, chr5:16179878, chr5:16179881, chr5:16179883, chr5:16180008, chr5:178204129, chr5:178204131, chr6:3577167, chr6:27279630, chr6:73310426, chr6:73310429, chr6:73310454, chr6:73310463, chr6:125791186, chr6:136290429, chr7:996193, chr7:1551166, chr7:1849861, chr7:1854236, chr7:4730195, chr7:5070131, chr7:6333296, chr7:15848453, chr7:26247657, chr7:29643153, chr7:30043812, chr7:43877706, chr7:44146254, chr7:44748739, chr7:149022546, chr8:31640044, chr8:31640053, chr8:31640067, chr8:42842942, chr8:80578027, chr9:107245787, chr9:120913537, chr9:126408197, chr9:131584010, chr9:136197462, chr9:136451036, chr9:136451222, chr9:136451519, chr9:137013461, chr9:137013470, chr9:137013473, chr9:137013486, chr9:137278360, chr9:137278384, chr9:137278402, chr9:137278488, chrX:150899022, chr6:34553001, chr12:118980854, chr19:811773, chr2:130345606, chr2:202250236, chr7:1005784, chr19:7934275, chr14:34024477, chr7:107390004, chr1:25216043, chr15:85610808, chr19:5668214, chr7:5297184, chr1:241356405, chr10:102714534, chr8:31640064, chr12:120967099, chr19:1393965, chr15:25438570, chr15:74789270, chr7:5297124, chr8:123443315, chr1:180269892, chr19:1456332, chr16:73028748, chr2:75056446, chr19:18543346, chr2:75056435, chr2:75056445, chr16:29876586, chr6:43771035, chr12:124344199, chr8:139649190, chr2:230068915, chr1:6461087, chr1:234661304, chr12:7824990, chr16:71931876, chr21:36736273, chr3:183636771, chr5:143405413, chr10:69402478, chr14:104887313, chr16:1315523, chr7:2295681, chr1:214108494, chr1:236064214, chr2:44168465, chr2:114662163, chr3:107999730, chr6:3577155, chr6:3577177, chr7:57818168, chr1:41324219, chr1:213051051, chr11:4521815, chr12:43552603, chr17:35488134, chr3:14401986, chr6:13450944, chr9:101738883, chr1:167221470, chr16:88880826, chr19:3652127, chr20:17396399, chr12:104599364, chr16:87827109, chr19:17873403, chr19:17873409, chr19:17873411, chr6:63656136, chr12:45216305, chr12:57837371, chr16:21557088, chr17:34331252, chr21:39054428, chr7:3910633, chr16:3655081, chr18:12324746, chr9:76701152, chr19:1648427, chr20:47468584, chr3:96841981, chr1:239386481, chr19:3088153, chr20:17396185, chr6:27557116, chr6:55680614, chr7:5297111, chr19:58440360, chr7:151111578, chr8:139649329, chr19:28520192, chr6:34778112, chr9:131112163, chr7:102201787, chr8:42153025, chr8:42622755, chr1:154174789, chr17:49816136, chr19:7934266, chr19:13207507, chr2:17317571, chr5:3754702, chr6:24626028, chr6:44653805, chr2:173307231, chr6:22509101, chr8:107889762, chr5:128879466, chr19:3094373, chr17:72235698, chr21:34510358, chr9:137013468, chr9:137013480, chr1:220001751, chr5:308909, chr1:155544582, chr1:204089544, chr11:107970310, chr12:95066688, chr19:16553992, chr5:16179995, chr5:16180003, chr5:25900907, chr8:35235230, chr16:28617820, chr17:76419894, chr19:48194427, chr7:14695759, chr1:6470978, chr1:23636028, chr20:350196, chr1:31193833, chr8:126862783, chr11:78046094, chr12:132528585, chr18:79375872, chr19:652099, chr21:9873175, chr7:57821358, chr7:101967581, chr8:144107705, chr12:62602516, chr1:6460819, chr1:244198034, chr19:5704451, chr9:137278356, chr10:76887333, chr19:1433766, chr19:13830675, chr20:37985816, chr1:15945048, chr14:23078131, chr9:133459357, chr8:31640070, chr12:62799867, chr5:32446706, chr12:120967103, chr5:128879583, chr15:69087040, chr20:347269, chr9:132325224, chr16:78195948, chr19:28824412, chr19:44753544, chr22:11288465, chr17:72123931, chr17:72124220, chr17:72124238, chr2:36973823, chr5:16179886, chr20:44560834, chr12:50057840, chr19:1456317, chr7:1421100, chr8:123443370, chr17:82626176, chr18:63283742, chr11:65650576, chr12:31591065, chr16:73028716, chr6:106135145, chr1:230399219, chr17:80599748, chr3:136614019, chr9:93826472, chr19:812407, chr6:135771171, chr9:137278500, chr16:3987194, chr17:49810078, chr2:164717792, chr22:40600900, chr3:123976907, chr3:123976934, chr2:89244905, chr19:5668194, chr11:78046073, chr9:127507984, chr9:120877473, chr1:159210159, chr6:44441882, chr8:101950035, chr1:11331701, chr19:1992998, chr19:1993003, chr9:128704978, chr9:124453638, chr19:1468243, chr16:2429260, chr20:62309824, chr7:44146237, chr8:31640050, chr9:94914976, chr12:37419025, chr12:45776142, chr17:42670166, chr19:1633551, chr9:137281710, chr1:214281447, chr6:34553086, chr1:167918494, chr17:72123590, chr19:45067160, chr6:157300486, chr7:1734991, chr7:1734996, chr1:70338695, chr19:18868002, chr9:14041343, chr10:76887324, chr19:7934351, chr19:13644740, chr2:5691280, chr5:134550960, chr12:95633568, chr1:6460956, chr16:17134583, chr16:17134595, chr16:61149835, chr19:4101376, chr19:37692212, chr6:1494847, chr7:38178231, chr7:71132295, chr11:134072686, chr16:2720277, chr16:11345580, chr17:29613358, chr18:14867181, chr19:3613344, chr2:209772188, chr6:34552991, chr6:88962788, chr12:105977085, chr17:7479367, chr17:21379559, chr19:10502839, chr19:39178093, chr21:43313606, chr22:31306646, chr3:11717633, chr3:139539693, chr7:4730228, chr4:154375969.

In some embodiments, a subject is classified as having SCLC-I. In some embodiments, a subject is classified as having SCLC-I based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, 900, or 1000 methylation sites of Table 5. Analyses of each and every specific combination of the methylation sites of Table 5 are contemplated herein. In some embodiments, a subject is classified as having SCLC-I based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 methylation sites of Table 10. Analyses of each and every specific combination of the methylation sites of Table 10 are contemplated herein. In some embodiments, a subject is classified as having SCLC-A based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, or 30 methylation sites of Table 18. Analyses of each and every specific combination of the methylation sites of Table 18 are contemplated herein. In some embodiments, a subject is classified as having SCLC-I based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 600, 700, 800, or 848 methylation sites of Table 23. Analyses of each and every specific combination of the methylation sites of Table 23 are contemplated herein. In some embodiments, SCLC-I is identified based on detection of differential methylation at at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 methylation sites of Table 30. Analyses of each and every specific combination of the methylation sites of Table 30 are contemplated herein. It is specifically contemplated that any one or more of the methylation sites of Table 30 may be excluded in embodiments described herein. In some embodiments, SCLC-I is identified based on detection of differential methylation of cg09248054, cg02379560, cg04917391, cg05020685, cg06485940, cg16364495, cg05857126, cg13780782, cg02008691, cg24664798, cg17265693, cg06508056, cg15932065, cg16405211, cg18804920, cg23889772, cg07270851, cg24238564, cg03850035, cg02659920, cg05651265, cg10976861, cg24127874, cg02365303, cg11799006, cg00336977, cg02125259, cg09433131, cg14865862, cg20136513, cg00378510, cg06473097, cg23844705, cg19741167, cg16769791, cg00448761, cg04303901, cg13077545, cg15559684, cg07035875, cg19115272, cg06093379, cg19154027, and/or cg22073838. In some embodiments, SCLC-I is identified based on detection of differential methylation of chr1:1040462, chr1:1040475, chr1:1682754, chr1:6249894, chr1:6249902, chr1:6249914, chr1:6249917, chr1:12191832, chr1:15741578, chr1:16980570, chr1:16980579, chr1:16980593, chr1:18630506, chr1:20487144, chr1:21574155, chr1:23801501, chr1:24730777, chr1:24730787, chr1:24730788, chr1:24730796, chr1:24730797, chr1:25195867, chr1:31065986, chr1:36323440, chr1:39491967, chr1:43285575, chr1:43285577, chr1:43369421, chr1:89843439, chr1:89843473, chr1:145960916, chr1:153532886, chr1:153532890, chr1:156908050, chr1:163404039, chr1:163423088, chr1:163423133, chr1:163423188, chr1:163423194, chr1:163423199, chr1:163546298, chr1:164136579, chr1:164136587, chr1:164136632, chr1:164236230, chr1:164236242, chr1:165235591, chr1:167796909, chr1:208601823, chr1:213771516, chr1:213820223, chr1:213861854, chr1:213861907, chr1:213910457, chr1:225962005, chr1:230322908, chr1:240992226, chr1:241128149, chr1:243916475, chr10:99431109, chr10:132518774, chr11:366066, chr11:438565, chr11:438607, chr11:726201, chr11:848890, chr11:848923, chr11:848927, chr11:848933, chr11:849115, chr11:849137, chr11:849141, chr11:849142, chr11:849148, chr11:849149, chr11:17734970, chr11:44566012, chr11:45922046, chr11:45922074, chr11:45922076, chr11:45922090, chr11:45922100, chr11:45922275, chr11:45922276, chr11:45923059, chr11:45923077, chr11:46381467, chr11:64299743, chr11:64299761, chr11:64300844, chr11:64300851, chr11:64300858, chr11:64300884, chr11:64490631, chr11:64490632, chr11:64712787, chr11:65355635, chr11:65355646, chr11:65355670, chr11:65551424, chr11:65551425, chr11:67023317, chr11:67464932, chr11:67464964, chr11:73264689, chr11:75240135, chr11:85811258, chr11:89593934, chr11:92709117, chr11:93542959, chr11:114059499, chr11:114059530, chr11:114059757, chr11:114061199, chr11:114061210, chr11:114061212, chr11:114061215, chr11:114061239, chr11:114631735, chr11:115504986, chr11:115504997, chr11:115505001, chr11:117432347, chr11:117795858, chr11:118956922, chr11:119182275, chr11:119206066, chr11:119729116, chr11:120284546, chr11:124746873, chr11:124746909, chr11:124746913, chr11:124863373, chr11:124863394, chr11:125263544, chr11:129316134, chr11:130070945, chr11:130159700, chr11:130159829, chr11:130159851, chr11:130159854, chr11:133064200, chr11:133064212, chr11:133064221, chr11:133064231, chr11:133064233, chr11:134311382, chr12:6961918, chr12:6962633, chr12:6962646, chr12:6962648, chr12:6964182, chr12:16504002, chr12:16714352, chr12:16714353, chr12:25939377, chr12:32470696, chr12:32646853, chr12:42238116, chr12:49346692, chr12:51392037, chr12:52903995, chr12:52904021, chr12:52948914, chr12:96738497, chr12:96738507, chr12:97138576, chr12:97324958, chr12:97325039, chr12:97607417, chr12:97619683, chr12:97755215, chr12:97855295, chr12:97924591, chr12:98350733, chr12:103100480, chr12:103165897, chr12:103165901, chr12:103165909, chr12:103165933, chr12:108310791, chr12:108310792, chr12:108310807, chr12:109291976, chr12:109292004, chr12:119258242, chr12:119258280, chr12:132134549, chr12:132134577, chr12:132134595, chr12:132134596, chr12:132134600, chr12:132134601, chr12:132134630, chr13:21298314, chr13:34918433, chr13:35920681, chr13:38494047, chr13:38545892, chr13:39205350, chr13:39300854, chr13:42040772, chr13:54129564, chr13:54844155, chr13:55129970, chr13:55166399, chr13:59341409, chr13:70033781, chr13:73632816, chr13:73632851, chr13:78840351, chr13:101796812, chr13:101927978, chr13:101933364, chr13:102060417, chr13:102060419, chr13:102060451, chr13:102068532, chr13:102068551, chr13:102068563, chr13:102068572, chr13:102390033, chr13:110473172, chr13:110536576, chr13:110825816, chr13:110830973, chr13:112988667, chr13:113016746, chr13:113433650, chr14:21069139, chr14:21069177, chr14:21069183, chr14:22883379, chr14:26553939, chr14:29450905, chr14:29450950, chr14:29523990, chr14:30391455, chr14:37657127, chr14:37751915, chr14:37751924, chr14:37751973, chr14:37751985, chr14:37772760, chr14:37772778, chr14:37772795, chr14:37802591, chr14:38033650, chr14:38033658, chr14:38115498, chr14:38115499, chr14:38115532, chr14:38129918, chr14:39442503, chr14:44156407, chr14:44156411, chr14:44156431, chr14:44379746, chr14:44379797, chr14:53956630, chr14:53956642, chr14:53956653, chr14:53956667, chr14:56506990, chr14:56534824, chr14:56832505, chr14:56992482, chr14:56992483, chr14:61348113, chr14:62736524, chr14:63064707, chr14:63114304, chr14:63114311, chr14:63114337, chr14:64741044, chr14:64942593, chr14:64942636, chr14:67241487, chr14:88791865, chr14:88791879, chr14:104827290, chr14:104895812, chr14:104895822, chr15:22702324, chr15:43776557, chr15:43776576, chr15:53791638, chr16:64387, chr16:561080, chr16:561087, chr16:677534, chr16:2148616, chr16:4260566, chr16:14303673, chr16:14303701, chr16:14303702, chr16:14303720, chr16:19522006, chr16:52395111, chr16:52395146, chr16:52504927, chr16:52504937, chr16:52504992, chr16:52621872, chr16:56191818, chr16:58500253, chr16:58500281, chr16:58500286, chr16:67666791, chr16:67666821, chr16:67666825, chr16:67666999, chr16:67667017, chr16:67667021, chr16:71626053, chr16:84312964, chr16:84312972, chr16:84312978, chr16:84519935, chr16:84659693, chr16:84842521, chr16:85449391, chr16:85449403, chr16:85449420, chr16:86467633, chr16:88291496, chr16:88302508, chr16:88737568, chr17:4263552, chr17:4449947, chr17:7044006, chr17:17403801, chr17:17403831, chr17:19379152, chr17:19379174, chr17:19379191, chr17:29176890, chr17:29565877, chr17:29565896, chr17:29565920, chr17:31561710, chr17:41527810, chr17:42951031, chr17:43720600, chr17:48892861, chr17:65625809, chr17:68870337, chr17:74213330, chr17:74961013, chr17:74961036, chr17:74972136, chr17:79794767, chr17:81397114, chr17:81397126, chr17:81397150, chr17:81514240, chr17:81902419, chr17:81902429, chr17:81902430, chr17:81902433, chr17:81902434, chr17:81902440, chr17:81902441, chr18:6929505, chr18:7327489, chr18:7327495, chr18:32928012, chr18:32928017, chr18:32928020, chr18:32928024, chr18:33167723, chr18:33167727, chr18:33225321, chr18:33317997, chr18:33318065, chr18:33570344, chr18:34609374, chr18:34639003, chr18:38714972, chr18:38714994, chr18:43102815, chr18:47748194, chr18:59148758, chr18:59148765, chr18:63207225, chr18:63207233, chr18:63207260, chr18:71409912, chr18:71652314, chr18:71652318, chr18:72662276, chr18:72677583, chr18:76446564, chr18:76446573, chr18:76446574, chr18:76446612, chr18:76472706, chr18:76580424, chr18:76590712, chr18:76590755, chr18:76590771, chr18:77020807, chr18:77132839, chr18:79334799, chr19:460731, chr19:460763, chr19:511206, chr19:511219, chr19:537165, chr19:1082036, chr19:1082073, chr19:1082079, chr19:1083180, chr19:2624624, chr19:4147445, chr19:4566632, chr19:4566647, chr19:4955146, chr19:6464556, chr19:6551811, chr19:6583884, chr19:7115981, chr19:7904264, chr19:9953412, chr19:9953424, chr19:9961699, chr19:9966503, chr19:9966579, chr19:9966586, chr19:9997385, chr19:10625441, chr19:10625699, chr19:11418683, chr19:13099153, chr19:13248768, chr19:14433259, chr19:15195814, chr19:15195823, chr19:15195830, chr19:15195831, chr19:15195838, chr19:15195844, chr19:15195847, chr19:15195851, chr19:17041847, chr19:17251851, chr19:28293220, chr19:29892627, chr19:33260980, chr19:33393584, chr19:36008846, chr19:36009562, chr19:42242259, chr19:43757320, chr19:44796859, chr19:45444316, chr19:45598430, chr19:46016118, chr19:46016119, chr19:46016123, chr19:46016124, chr19:46016125, chr19:46016126, chr19:46016130, chr19:46016136, chr19:46016138, chr19:46016139, chr19:46016142, chr19:46016144, chr19:46016145, chr19:46016146, chr19:46016147, chr19:46016148, chr19:46016149, chr19:46016465, chr19:48752569, chr19:48752624, chr19:48752637, chr19:48753115, chr19:48753117, chr19:48753159, chr19:48753363, chr19:48753390, chr19:50417522, chr19:50418925, chr19:51030848, chr19:51077450, chr19:52028350, chr19:53991315, chr19:55374611, chr19:55554857, chr2:3303753, chr2:4107720, chr2:4798818, chr2:6987052, chr2:7032109, chr2:8457740, chr2:14507507, chr2:25190664, chr2:47369968.

In some embodiments, methylation status of various methylation sites (e.g., methylation sites of Tables 1, 2.3.4.5.6.7, 8, 9, 10, 15, 16, 17, 18, 19, 20, 21, 22, 23, 24, 26, 27, 28, 29, and/or 30) is analyzed from tumor DNA from the subject. In some embodiments, tumor DNA is obtained or derived from a tissue sample from the subject. In some embodiments, tumor DNA is obtained or derived from a blood sample from a subject. In some embodiments, tumor DNA is obtained or derived from a plasma sample from a subject. In some embodiments, the tumor DNA is circulating tumor DNA (ctDNA).

Following classification of a subject as having SCLC-A, SCLC-N, SCLC-P, or SCLC-I, the subject may be administered one or more cancer therapies. Example cancer therapies useful for treatment of specific SCLC subtypes are described elsewhere herein.

### III. Detection, Diagnosis, and Monitoring

Aspects of the present disclosure comprise diagnosis of a subject with small cell lung cancer (SCLC). In some embodiments, disclosed are methods for diagnosing a subject with SCLC. In some embodiments, disclosed are methods for identifying a subject with cancer as having SCLC. For example, certain aspects are directed to methods for identifying a subject as having SCLC comprising determining the subject to have differential methylation of one or more methylation sites of Table 13, based on analysis of DNA from the subject. A subject may be a subject having cancer. A subject may be as subject suspected of having cancer. A subject may have an unknown cancer type. A subject may have lung cancer of an unknown type, where the disclosed methods are useful in identifying the subject as having SCLC and not as having non-small cell lung cancer (NSCLC).

In some embodiments, the subject is determined to have SCLC based on analysis of at least or at most 1, 2, 3, 4, 5, 6, 7, 8, 9, 10, 11, 12, 13, 14, 15, 16, 17, 18, 19, 20, 21, 22, 23, 24, 25, 26, and 27 methylation sites of Table 13. Methylation sites may be analyzed from DNA from the subject. In some embodiments, the DNA is tumor DNA. In some embodiments, the DNA is circulating tumor DNA (ctDNA). Analyses of each and every combination of methylation sites from Table 13 are contemplated herein. For example, a subject may be determined to have differential methylation of 2, 3, 4, 5, 6, 7, 8, 9, 10, 11, 12, 13, 14, 15, 16, 17, 18, 19, 20, 21, 22, 23, 24, 25, 26, or 27 of the methylation sites of Table 13, thereby identifying the subject as having SCLC. In some embodiments, the disclosed methods comprise determining, based on analysis of tumor DNA from a subject, the subject to have differential methylation at cg09052983, cg03196720, cg03851835, cg23847017, cg06029700, cg16955166, cg00956142, cg07101841, cg22099241, cg00233633, cg02339793, cg07093324, cg18166947, cg21055554, cg18474885, cg19166875, cg24473500, cg22234930, cg23715728, cg04650676, cg00134210, cg04387396, cg01807820, cg15689991, cg03577157, cg11708454, and/or cg08962271.

After identifying the subject as having SCLC, the subject may be administered one or more SCLC therapies. SCLC therapies are known in the art, and certain examples are described herein.

Additional aspects of the disclosure relate to evaluation of tumor burden in a subject having SCLC, monitoring SCLC treatment efficacy, and evaluating and adjusting SCLC treatment strategy. As described herein, certain methylation sites, including the methylation sites of Table 24 (chr5:77844815, chr5:77844832, chr5:77844821, chr5:132257679, chr16:49280789, chr21:34670607, chr21:34670604, chr21:34670609, chr6:108176627, chr10:71638792, chr5:132257648, chr5:132257653, chr5:132257685, chr5:132257670, chr5:138274351, chr8:22139065, chr5:132257664, chr5:132257652, chr5:132257666, chr3:38039198, chr9:137591221, chr9:131444459, chr6:108176609, chr5:132257661, chr5:132257650, chr5:132257703, chr5:132257682, chr5:178860732, chr5:132257676, chr20:22582930, chr2:10043081, chr2:98347974, chr5:132257649, chr5:178860742, chr5:138274357, chr5:138274360, chr10:75408778, chr5:132257647, chr5:132257637, chr6:108176612, chr22:50251762, chr21:34669575) correlate with general tumor burden in SCLC and thus can be used to evaluate tumor burden and monitor treatment response. Accordingly, disclosed are methods comprising determining, from DNA from a subject, a methylation status of two or more methylation sites of Table 24. A methylation status may be determined for at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of the methylation sites of Table 24. In some aspects, disclosed are methods comprising determining a methylation status of at least or at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 of the following methylation sites: chr5:77844815, chr5:77844832, chr5:77844821, chr5:132257679, chr16:49280789, chr21:34670607, chr21:34670604, chr21:34670609, chr6:108176627, chr10:71638792, chr5:132257648, chr5:132257653, chr5:132257685, chr5:132257670, chr5:138274351, chr8:22139065, chr5:132257664, chr5:132257652, chr5:132257666, chr3:38039198, chr9:137591221, chr9:131444459, chr6:108176609, chr5:132257661, chr5:132257650, chr5:132257703, chr5:132257682, chr5:178860732, chr5:132257676, chr20:22582930, chr2:10043081, chr2:98347974, chr5:132257649, chr5:178860742, chr5:138274357, chr5:138274360, chr10:75408778, chr5:132257647, chr5:132257637, chr6:108176612, chr22:50251762, and chr21:34669575. It is specifically contemplated that any one or more of these methylation sites may be excluded from certain embodiments.

In some aspects, the disclosed methods comprise evaluating tumor DNA from a subject having SCLC who is currently receiving or has previously received SCLC therapy. Also disclosed are treatment methods comprising determining a methylation level of two or more methylation sites of Table 24, administering a cancer therapy to a subject, then determining an additional methylation level of the same two or more methylation sites of Table 24 and comparing the methylation levels. A decreased methylation level may indicate a reduced tumor burden and, thus, that the cancer therapy is effective. An increased or unchanged methylation level may indicate an increased or unchanged tumor burden and, thus, that the cancer therapy is ineffective. Accordingly, if a decreased methylation level is measured following treatment, the same treatment may be continued, while if an increased or unchanged methylation level is measured following treatment, a different treatment may be selected and administered.

### IV. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy, or skin biopsy. In certain embodiments the sample is obtained from a biopsy from lung tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, plasma, serum, pleural fluid, pericardial fluid, spinal fluid, ascitic fluid, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. A sample may also include a sample devoid of cells, for example a cell-free sample comprising cell-free nucleic acid, such as a serum sample. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, blood collection, plasma collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple lung samples or multiple blood or plasma samples, may be obtained for diagnosis by the methods described herein. In other cases, multiple samples, such as one or more samples from one tissue type (for example lung) and one or more samples from another specimen (for example serum) may be obtained for diagnosis by the methods. In some cases, multiple samples such as one or more samples from one tissue type (e.g. lung) and one or more samples from another specimen (e.g. serum) may be obtained at the same or different times.

In some embodiments, a biological sample analyzed here is a liquid sample. In some embodiments, the sample is a blood sample. In some embodiments, the sample is a plasma sample. In some embodiments, the sample is a serum sample. A liquid sample may comprise tumor DNA. As discussed herein, “tumor DNA” describes any DNA derived from a tumor, and includes tumor DNA derived from a solid tumor sample (e.g., a solid biopsy) and tumor DNA obtained from cell-free sample (e.g., plasma, blood, etc.). Tumor DNA from a liquid sample may be cell-free DNA (cfDNA) and/or DNA from circulating tumor cells. As described herein. “circulating tumor DNA,” or “ctDNA” describes tumor DNA obtained from blood or a blood component (e.g., plasma, serum) from a subject. Tumor DNA, including circulating tumor DNA (ctDNA), may be isolated from a sample and analysed as disclosed herein (e.g., by sequcing such as bisulfite sequencing).

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### V. Assay Methods

A. Detection of Methylated DNA

Aspects of the methods include assaying nucleic acids (e.g., tumor DNA) to determine expression levels and/or methylation levels of nucleic acids. In some embodiments, disclosed are methods comprising determining a methylation status of one or more methylation sites from methylated DNA. The disclosed methods may comprise determining a subject (i.e., DNA from a subject such as tumor DNA) to have differential methylation at one or more methylation sites. As used herein, “differential methylation” of a methylation site describes a significant difference in methylation status of the methylation site in a sample (e.g., a sample comprising tumor DNA from a subject having cancer) as compared to a control or reference (e.g., DNA from a healthy subject). For example, in some embodiments, a methylation site from a sample comprising tumor DNA has significantly increased methylation levels compared to the same methylation site from control (e.g., healthy, non-tumor) DNA. In some embodiments, a methylation site from a sample comprising tumor DNA has significantly decreased methylation levels compared to the same methylation site from control (e.g., healthy, non-tumor) DNA. Assays for the detection of methylated DNA are known in the art. Methylated DNA includes, for example, methylated circulating tumor DNA. Certain, non-limiting examples of such methods are described herein.

1. HPLC-UV

The technique of HPLC-UV (high performance liquid chromatography-ultraviolet), developed by Kuo and colleagues in 1980 (described further in Kuo K. C. et al., Nucleic Acids Res. 1980; 8:4763-4776, which is herein incorporated by reference) can be used to quantify the amount of deoxycytidine (dC) and methylated cytosines (5 mC) present in a hydrolysed DNA sample. The method includes hydrolyzing the DNA into its constituent nucleoside bases, the 5 mC and dC bases are separated chromatographically and, then, the fractions are measured. Then, the 5 mC/dC ratio can be calculated for each sample, and this can be compared between the experimental and control samples.

2. LC-MS/MS

Liquid chromatography coupled with tandem mass spectrometry (LC-MS/MS) is an high-sensitivity approach to HPLC-UV, which requires much smaller quantities of the hydrolysed DNA sample. In the case of mammalian DNA, of which ˜2%-5% of all cytosine residues are methylated, LC-MS/MS has been validated for detecting levels of methylation levels ranging from 0.05%-10%, and it can confidently detect differences between samples as small as ˜0.25% of the total cytosine residues, which corresponds to ˜5% differences in global DNA methylation. The procedure routinely requires 50-100 ng of DNA sample, although much smaller amounts (as low as 5 ng) have been successfully profiled. Another major benefit of this method is that it is not adversely affected by poor-quality DNA (e.g., DNA derived from FFPE samples).

3. ELISA-Based Methods

There are several commercially available kits, all enzyme-linked immunosorbent assay (ELISA) based, that enable the quick assessment of DNA methylation status. These assays include Global DNA Methylation ELISA, available from Cell Biolabs; Imprint Methylated DNA Quantification kit (sandwich ELISA), available from Sigma-Aldrich; EpiSeeker methylated DNA Quantification Kit, available from abcam; Global DNA Methylation Assay—LINE-1, available from Active Motif: 5-mC DNA ELISA Kit, available from Zymo Research: MethylFlash Methylated DNAS-mC Quantification Kit and MethylFlash Methylated DNAS-mC Quantification Kit, available from Epigentek.

Briefly, the DNA sample is captured on an ELISA plate, and the methylated cytosines are detected through sequential incubations steps with: (1) a primary antibody raised against 5 Mc; (2) a labelled secondary antibody: and then (3) colorimetric/fluorometric detection reagents.

The Global DNA Methylation Assay—LINE-1 specifically determines the methylation levels of LINE-1 (long interspersed nuclear elements-1) retrotransposons, of which ˜17% of the human genome is composed. These are well established as a surrogate for global DNA methylation. Briefly, fragmented DNA is hybridized to biotinylated LINE-1 probes, which are then subsequently immobilized to a streptavidin-coated plate. Following washing and blocking steps, methylated cytosines are quantified using an anti-5 mC antibody, HRP-conjugated secondary antibody and chemiluminescent detection reagents. Samples are quantified against a standard curve generated from standards with known LINE-1 methylation levels. The manufacturers claim the assay can detect DNA methylation levels as low as 0.5%. Thus, by analyzing a fraction of the genome, it is possible to achieve better accuracy in quantification.

4. LINE-1 Pyrosequencing

Levels of LINE-1 methylation can alternatively be assessed by another method that involves the bisulfite conversion of DNA, followed by the PCR amplification of LINE-1 conservative sequences. The methylation status of the amplified fragments is then quantified by pyrosequencing, which is able to resolve differences between DNA samples as small as ˜5%. Even though the technique assesses LINE-1 elements and therefore relatively few CpG sites, this has been shown to reflect global DNA methylation changes very well. The method is particularly well suited for high throughput analysis of cancer samples, where hypomethylation is very often associated with poor prognosis. This method is particularly suitable for human DNA, but there are also versions adapted to rat and mouse genomes.

5. AFLP and RFLP

Detection of fragments that are differentially methylated could be achieved by traditional PCR-based amplification fragment length polymorphism (AFLP), restriction fragment length polymorphism (RFLP) or protocols that employ a combination of both.

6. LUMA

The LUMA (luminometric methylation assay) technique utilizes a combination of two DNA restriction digest reactions performed in parallel and subsequent pyrosequencing reactions to fill-in the protruding ends of the digested DNA strands. One digestion reaction is performed with the CpG methylation-sensitive enzyme HpaII; while the parallel reaction uses the methylation-insensitive enzyme MspI, which will cut at all CCGG sites. The enzyme EcoRI is included in both reactions as an internal control. Both MspI and HpaII generate 5′-CG overhangs after DNA cleavage, whereas EcoRI produces 5′-AATT overhangs, which are then filled in with the subsequent pyrosequencing-based extension assay. Essentially, the measured light signal calculated as the HpaII/MspI ratio is proportional to the amount of unmethylated DNA present in the sample. As the sequence of nucleotides that are added in pyrosequencing reaction is known, the specificity of the method is very high and the variability is low, which is essential for the detection of small changes in global methylation. LUMA requires only a relatively small amount of DNA (250-500 ng), demonstrates little variability and has the benefit of an internal control to account for variability in the amount of DNA input.

7. Bisulfite Sequencing

The bisulfite treatment of DNA mediates the deamination of cytosine into uracil, and these converted residues will be read as thymine, as determined by PCR-amplification and subsequent Sanger sequencing analysis. However, 5 mC residues are resistant to this conversion and, so, will remain read as cytosine. Thus, comparing the Sanger sequencing read from an untreated DNA sample to the same sample following bisulfite treatment enables the detection of the methylated cytosines. With the advent of next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5 mC-enriched fraction of the genome is not only a less expensive approach, but it also allows one to increase the sequencing coverage and, therefore, precision in revealing differentially-methylated regions. Sequencing could be done using any existing NGS platform; Illumina and Life Technologies both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after MspI digestion that recognizes CCGG sites (and it cut both methylated and unmethylated sites). It ensures isolation of ˜85% of CpG islands in the human genome. Then, the same bisulfite conversion and library preparation is performed as for WGBS. The RRBS procedure normally requires ˜100 ng-1 μg of DNA.

8. Methods that Exclude Bisulfite Conversion

In some aspects, direct detection of modified bases without bisulfite conversion may be used to detect methylation. For example, Pacific Biosciences company has developed a way to detect methylated bases directly by monitoring the kinetics of polymerase during single molecule sequencing and offers a commercial product for such sequencing (further described in Flusberg B. A., et al., Nat. Methods. 2010; 7:461-465, which is herein incorporated by reference). Other methods include nanopore-based single-molecule real-time sequencing technology (SMRT), which is able to detect modified bases directly (described in Laszlo A. H. et al., Proc. Natl. Acad. Sci. USA. 2013 and Schreiber J., et al., Proc. Natl. Acad. Sci. USA. 2013, which are herein incorporated by reference).

9. Array or Bead Hybridization

Methylated DNA fractions of the genome, usually obtained by immunoprecipitation, could be used for hybridization with microarrays. Currently available examples of such arrays include: the Human CpG Island Microarray Kit (Agilent), the GeneChip Human Promoter 1.0R Array and the GeneChip Human Tiling 2.0R Array Set (Affymetrix).

The search for differentially-methylated regions using bisulfite-converted DNA could be done with the use of different techniques. Some of them are easier to perform and analyse than others, because only a fraction of the genome is used. The most pronounced functional effect of DNA methylation occurs within gene promoter regions, enhancer regulatory elements and 3′ untranslated regions (3′UTRs). Assays that focus on these specific regions, such as the Infinium HumanMethylation450 Bead Chip array by Illumina, can be used. The arrays can be used to detect methylation status of genes, including miRNA promoters, 5′ UTR, 3′ UTR, coding regions (˜17 CpG per gene) and island shores (regions ˜2 kb upstream of the CpG islands).

Briefly, bisulfite-treated genomic DNA is mixed with assay oligos, one of which is complimentary to uracil (converted from original unmethylated cytosine), and another is complimentary to the cytosine of the methylated (and therefore protected from conversion) site. Following hybridization, primers are extended and ligated to locus-specific oligos to create a template for universal PCR. Finally, labelled PCR primers are used to create detectable products that are immobilized to bar-coded beads, and the signal is measured. The ratio between two types of beads for each locus (individual CpG) is an indicator of its methylation level.

It is possible to purchase kits that utilize the extension of methylation-specific primers for validation studies. In the VeraCode Methylation assay from Illumina, 96 or 384 user-specified CpG loci are analysed with the GoldenGate Assay for Methylation. Differently from the BeadChip assay, the VeraCode assay requires the BeadXpress Reader for scanning.

10. Methyl-Sensitive Cut Counting: Endonuclease Digestion Followed by Sequencing

As an alternative to sequencing a substantial amount of methylated (or unmethylated) DNA, one could generate snippets from these regions and map them back to the genome after sequencing. The technique of serial analysis of gene expression (SAGE) has been adapted for this purpose and is known as methylation-specific digital karyotyping, as well as a similar technique, called methyl-sensitive cut counting (MSCC).

In summary, in all of these methods, methylation-sensitive endonuclease(s), e.g., HpaII is used for initial digestion of genomic DNA in unmethylated sites followed by adaptor ligation that contains the site for another digestion enzyme that is cut outside of its recognized site, e.g., EcoP15I or MmeI. These ways, small fragments are generated that are located in close proximity to the original HpaII site. Then. NGS and mapping to the genome are performed. The number of reads for each HpaII site correlates with its methylation level.

A number of restriction enzymes have been discovered that use methylated DNA as a substrate (methylation-dependent endonucleases). Most of them were discovered and are sold by SibEnzyme: BisI, BIsI, GlaI. GluI, KroI, MteI, PcsI, PkrI. The unique ability of these enzymes to cut only methylated sites has been utilized in the method that achieved selective amplification of methylated DNA. Three methylation-dependent endonucleases that are available from New England Biolabs (FspEI, MspJI and LpnPI) are type IIS enzymes that cut outside of the recognition site and, therefore, are able to generate snippets of 32 bp around the fully-methylated recognition site that contains CpG. These short fragments could be sequences and aligned to the reference genome. The number of reads obtained for each specific 32-bp fragment could be an indicator of its methylation level. Similarly, short fragments could be generated from methylated CpG islands with Escherichia coli's methyl-specific endonuclease McrBC, which cuts DNA between two half-sites of (G/A) mC that are lying within 50 bp-3000 bp from each other. This is a very useful tool for isolation of methylated CpG islands that again can be combined with NGS. Being bisulfite-free, these three approaches have a great potential for quick whole genome methylome profiling.

B. Sequencing

DNA, including bisulfite-converted DNA, may be used for amplification of a region of interest followed by sequencing. Primers may designed around a methylation site of interest and used for PCR amplification of bisulfite-converted DNA. The resulting PCR products may be cloned and sequenced. Accordingly, aspects of the disclosure may include sequencing nucleic acids to detect methylation of nucleic acids and/or biomarkers. In some embodiments, the methods of the disclosure include a sequencing method. Example sequencing methods include those described below.

1. Massively parallel signature sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex. MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine. The essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing.

3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massivelly parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

5. Solid Sequencing.

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent Semiconductor Sequencing.

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

### VI. Cancer Therapy

In some embodiments, the disclosed methods comprise administering a cancer therapy to a subject or patient. The cancer therapy may be chosen based on expression level measurements, methylation status measurements, and/or other factors such as a clinical risk score calculated for the subject. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be a checkpoint inhibitor therapy. Any of these cancer therapies may also be excluded. Combinations of these therapies may also be administered.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

In some embodiments, disclosed are methods for treating cancer originating from the lung. In some embodiments, the cancer is lung cancer. In some embodiments, the cancer is small cell lung cancer (SCLC). In some embodiments, disclosed are methods for treating high-grade neuroendocrine carcinomas.

A. Chemotherapies

In some embodiments, methods of the disclosure comprise administering a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent. Other suitable chemotherapeutic agents include antimicrotubule agents, e.g., Paclitaxel (“Taxol”) and doxorubicin hydrochloride (“doxorubicin”).

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

The amount of the chemotherapeutic agent delivered to the patient may be variable. In one suitable embodiment, the chemotherapeutic agent may be administered in an amount effective to cause arrest or regression of the cancer in a host. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

B. Surgery

In some embodiments, the disclosed methods comprise surgery. Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an anti-cancer therapy, such as a chemotherapeutic. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

C. Immunotherapy

In some embodiments, the disclosed methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below. In some embodiments, a cancer immunotherapy is administered to a subject having been determined to have a cancer of the SCLC-I subtype. In some embodiments, a cancer immunotherapy is administered to a subject having been determined to have a cancer of the SCLC-A subtype. In some embodiments, a cancer immunotherapy is administered to a subject having been determined to have a cancer of the SCLC-N subtype. In some embodiments, a cancer immunotherapy is administered to a subject having been determined to have a cancer of the SCLC-P subtype. In some embodiments, a cancer immunotherapy is administered to a subject in combination with one or more additional cancer therapies.

1. Checkpoint Inhibitors and Combination Treatment

Embodiments of the disclosure may include administration of immune checkpoint inhibitors, which are further described below.

a. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1. PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein. CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998: can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1. B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

c. LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells. B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8+ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu. X., et al., mAbs, 2019: 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

d. TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD366. The complete mRNA sequence of human TIM-3 has the Genbank accession number NM_032782. TIM-3 is found on the surface IFNγ-producing CD4+Th1 and CD8+Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to TIM-3 also can be used.

In some embodiments, the inhibitor comprises the heavy and light chain CDRs or VRs of an anti-TIM-3 antibody. Accordingly, in one embodiment, the inhibitor comprises the CDR1. CDR2, and CDR3 domains of the V_{H} region of an anti-TIM-3 antibody, and the CDR1, CDR2 and CDR3 domains of the V_{L} region of an anti-TIM-3 antibody. In another embodiment, the antibody has at least about 70, 75, 80, 85, 90, 95, 97, or 99% (or any derivable range or value therein) variable region amino acid sequence identity with the above-mentioned antibodies.

2. Activation of Co-Stimulatory Molecules

In some embodiments, the immunotherapy comprises an activator of a co-stimulatory molecule. In some embodiments, the activator comprises an agonist of B7-1 (CD80), B7-2 (CD86), CD28, ICOS, OX40 (TNFRSF4), 4-1BB (CD137: TNFRSF9), CD40L (CD40LG), GITR (TNFRSF18), and combinations thereof. Activators include agonistic antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

4. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell, NK cell, or other immune cell. The receptors are called chimeric because they are fused of parts from different sources. CAR-T cell therapy refers to a treatment that uses such transformed T-cells for cancer therapy. Similarly, CAR-NK cell therapy refers to a treatment that uses such transformed NK cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

5. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fail in three groups: type I (IFNα and IFNβ), type II (IFNγ) and type III (IFNλ).

Interleukins have an array of immune system effects. IL-2 is an exemplary interleukin cytokine therapy.

6. Adoptive T-cell therapy

Adoptive T cell therapy is a form of passive immunization by the transfusion of T-cells (adoptive cell transfer). They are found in blood and tissue and usually activate when they find foreign pathogens. Specifically they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumour death.

Multiple ways of producing and obtaining tumour targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein. VII. Administration of Therapeutic Combinations

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

Embodiments of the disclosure relate to compositions and methods comprising therapeutic compositions. Different therapies may be administered in one composition or in more than one composition, such as 2 compositions, 3 compositions, or 4 compositions. Various combinations of the agents may be employed.

Therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. Kits

Certain aspects of the present disclosure also concern kits containing compositions of the disclosure or compositions to implement methods disclosed herein. In some embodiments, kits can be used to evaluate one or more biomarkers, such as methylation levels. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, there are kits for evaluating methylation levels of tumor DNA.

Kits may comprise components, which may be individually packaged or placed in a container, such as a tube, bottle, vial, syringe, or other suitable container means.

Individual components may also be provided in a kit in concentrated amounts: in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

In certain aspects, negative and/or positive control nucleic acids, probes, and inhibitors are included in some kit embodiments. In addition, a kit may include a sample that is a negative or positive control for methylation of one or more biomarkers. In some embodiments, a control includes a nucleic acid that contains at least one CpG or is capable of identifying a CpG methylation site.

Any embodiment of the disclosure involving specific biomarker by name is contemplated also to cover embodiments involving biomarkers whose sequences are at least 80, 81, 82, 83, 84, 85, 86, 87, 88, 89, 90, 91, 92, 93, 94, 95, 96, 97, 98, 99% identical to the mature sequence of the specified nucleic acid.

Embodiments of the disclosure include kits for analysis of a pathological sample by assessing biomarker profile for a sample comprising, in suitable container means, two or more biomarker probes, wherein the biomarker probes detect one or more of the biomarkers identified herein. The kit can further comprise reagents for labeling nucleic acids in the sample. The kit may also include labeling reagents, including at least one of amine-modified nucleotide, poly(A) polymerase, and poly(A) polymerase buffer. Labeling reagents can include an amine-reactive dye.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate certain embodiments of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1—Classification of SCLC Based on Methylation Markers Derived from Cell Lines

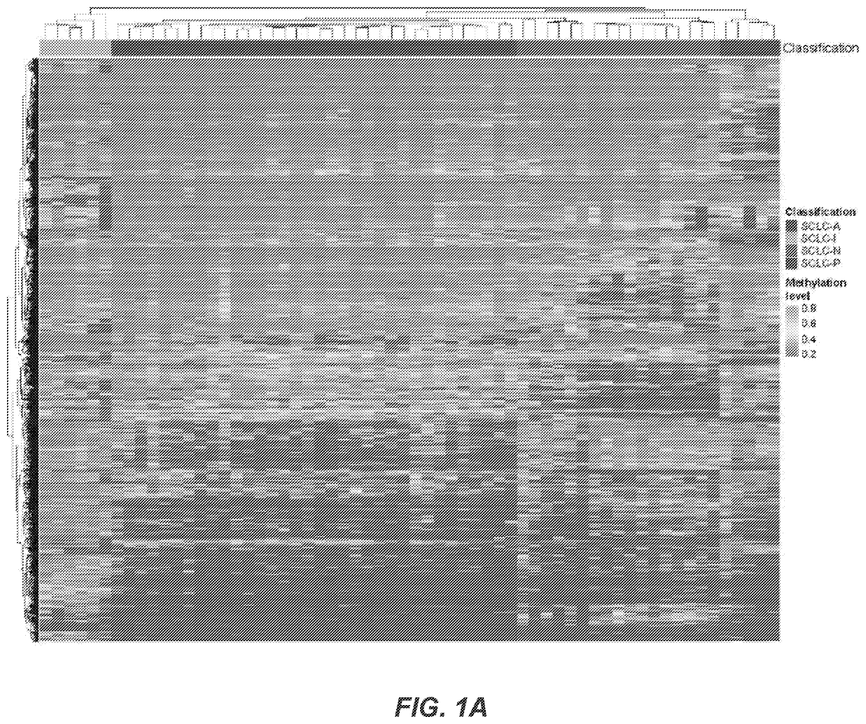

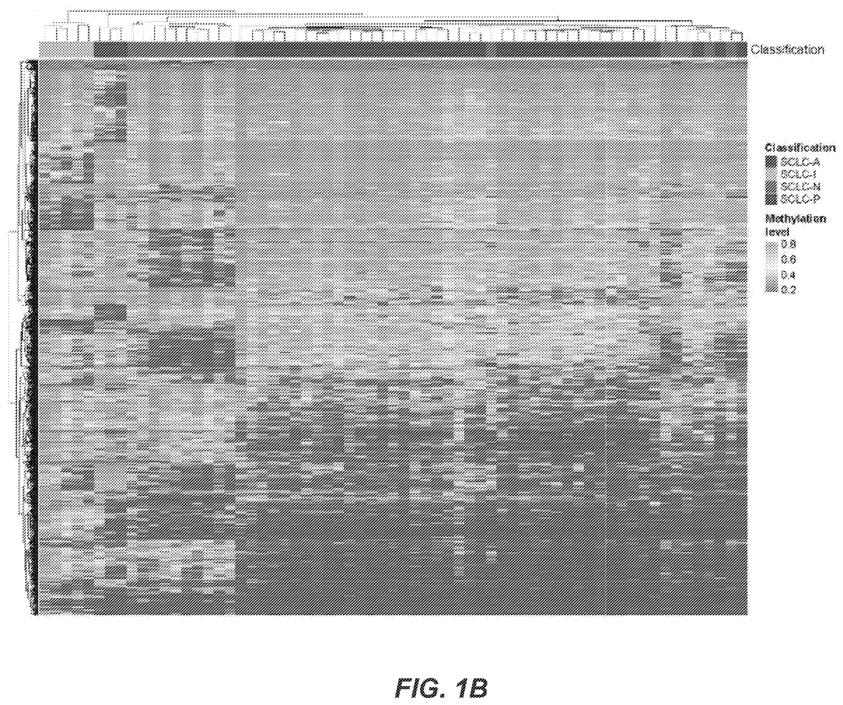

For the identification of distinct methylation sites, two published datasets were used, derived from the GDSC project (described in Iorio et al., Cell. 2016 Jul. 28; 166(3):740-754. doi: 10.1016/j.cell.2016.06.017, incorporated herein by reference in its entirety) and the NCI DTP SCLC project (available from the World Wide Web at sclccelflines.cancer.gov/sclc/). Association of methylation sites with SCLC subtype was assessed using Area Under the Receiver Operating Characteristics (AUROC) and the top 15,000 Methylation sites were used for further analysis. The general feasibility of using methylation to distinguish SCLC-subtypes is highlighted in FIGS. 1A-1B.

The clustering in FIGS. 1A-1B revealed that the top 1000 methylation sites were indeed able to distinguish the different subtypes, however clustering separated the subtypes less in the NCI dataset (FIG. 1B) than in the GDSC dataset (FIG. 1A). Consequently, a machine learning based algorithm was used to improve classification of the subtypes. Those models were validated only for the SCLC-A and SCLC-N subtype across the two datasets as there was limited data present for the SCLC-P and the SCLC-I dataset. However, several markers with an AUROC=1 have been identified in the latter two subsets highlighting their validity to distinguish the subsets.

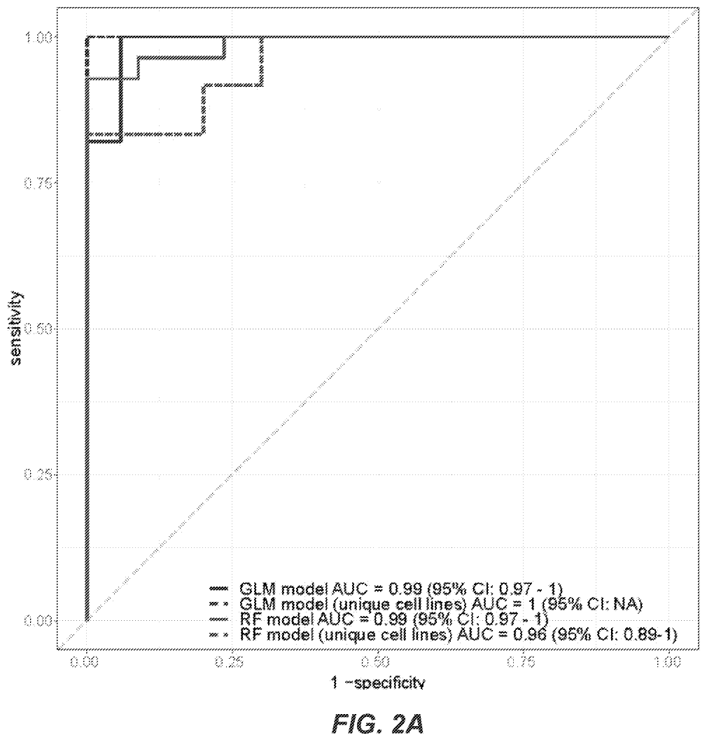

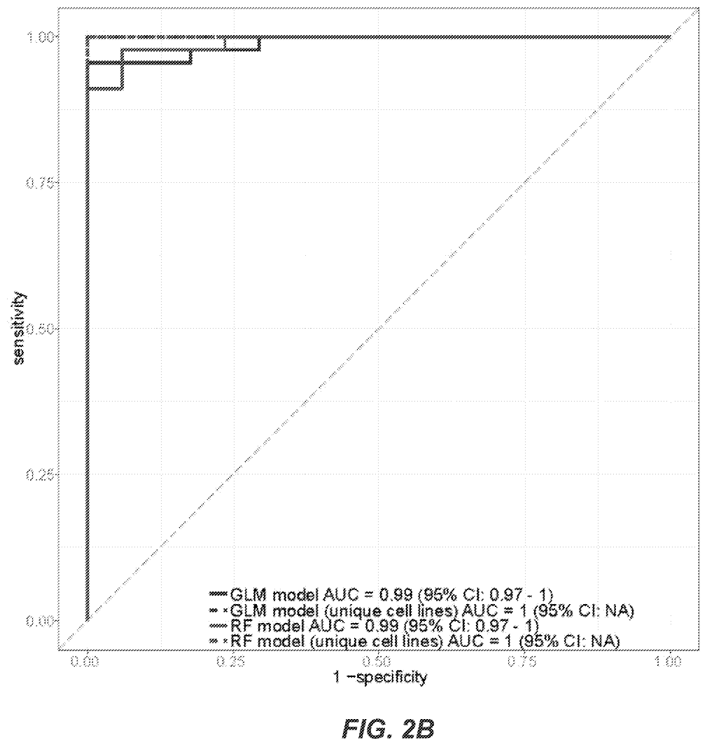

Importantly, the models demonstrated an impressive prediction for distinguishing the SCLC-A (FIG. 2A) and the SCLC-N subtypes (FIG. 2B). Interestingly, models generated using net-elastic logistic regression outperformed the models built using the random forest approach. Importantly, those results clearly highlight that methylation-based markers can be used for the classification of SCLC subtypes.

The differentiation of the respective subsets using a large number of methylation markers was demonstrated; however, the inventors hypothesized that a comparable prediction of the SCLC subset was also possible with a dramatically reduced set of methylation sites using only 2 instead of 15,000 methylation sites per subtype. Therefore, the most promising methylation sites (top 20 sites based on ROC analysis) were selected and all possible combinations of two markers were tested to assess if this approach was feasible to reduce the marker set.

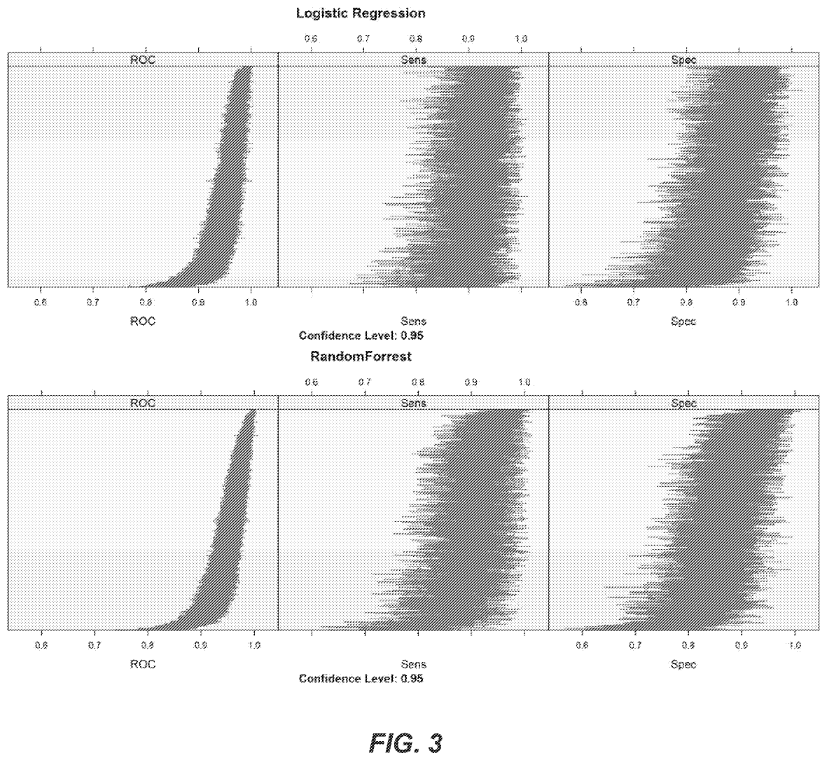

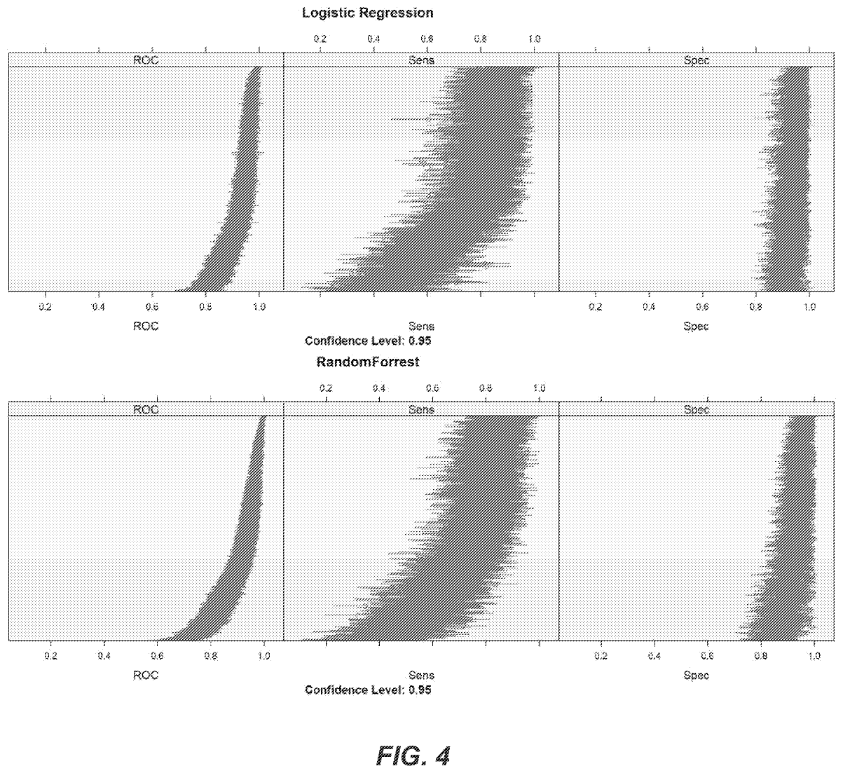

Importantly, as highlighted in FIG. 3 for the SCLC-A models and FIG. 4 for SCLC-N models, the approach of reducing the marker set to only two per subtypes was very successful in the SCLC-A and SCLC-N subtypes demonstrating that a reduced marker set was indeed capable of distinguishing the subtypes with comparable predictive performance. The top ten two-gene combinations for the SCLC-A and SCLC-N subtypes are provided in Tables 11 and 12.

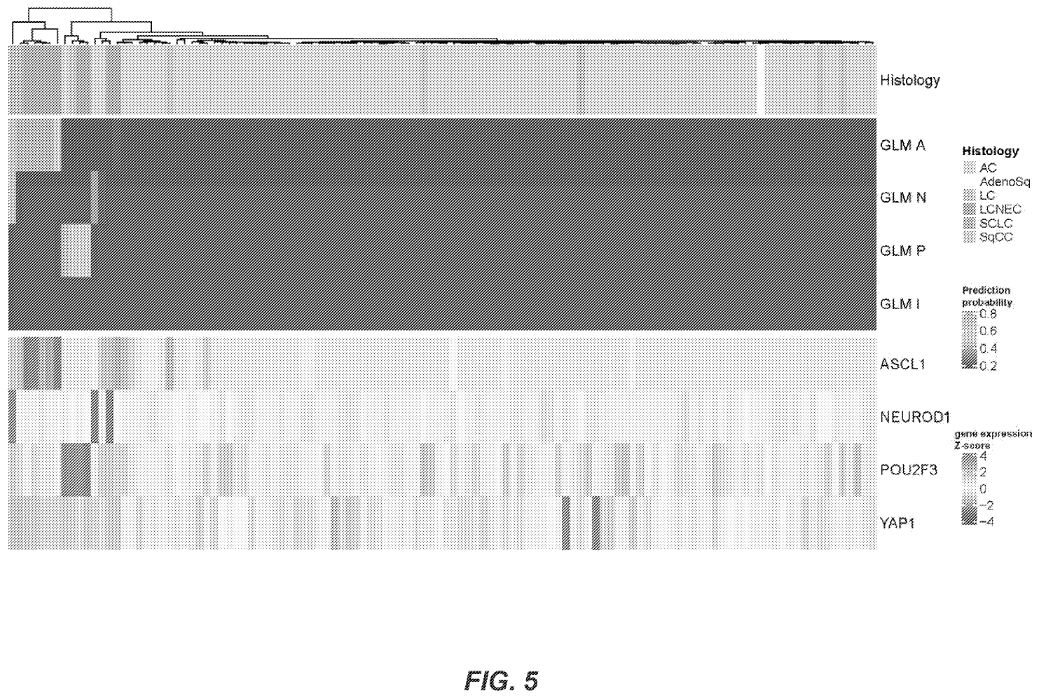

The models were initially exclusively trained on cell line samples and thus it was important to confirm how well they generalized over real tumor samples. Additionally, was is important to assess how the models compare on other tumor samples. Prediction on tumor samples was performed with coexisting gene expression data using the GSE56044 dataset. Prediction of subtypes was done using the models trained on the cell line data, which had never seen original tumor data. Results are highlighted in FIG. 5.

As seen in FIG. 5, the created models indeed generalized very well and were able to classify both SCLC and other HGNEC, such as LCNEC/LC. The results of the subgroups were concordant with the expression of the underlying markers highlighting the good reliability. Importantly, non-SCLC/LCNEC tumors showed no signal highlighting a certain specificity for SCLC/LCNEC.

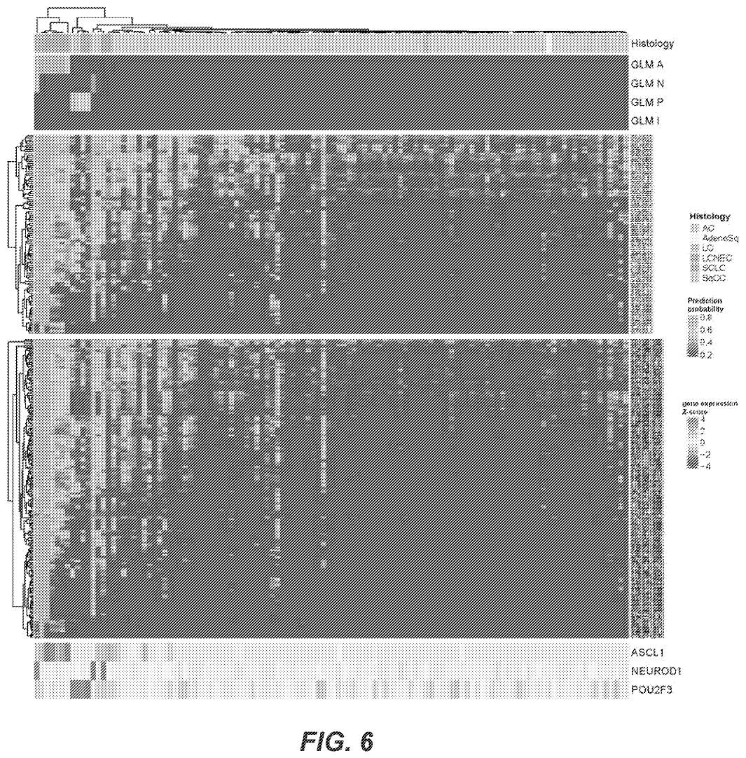

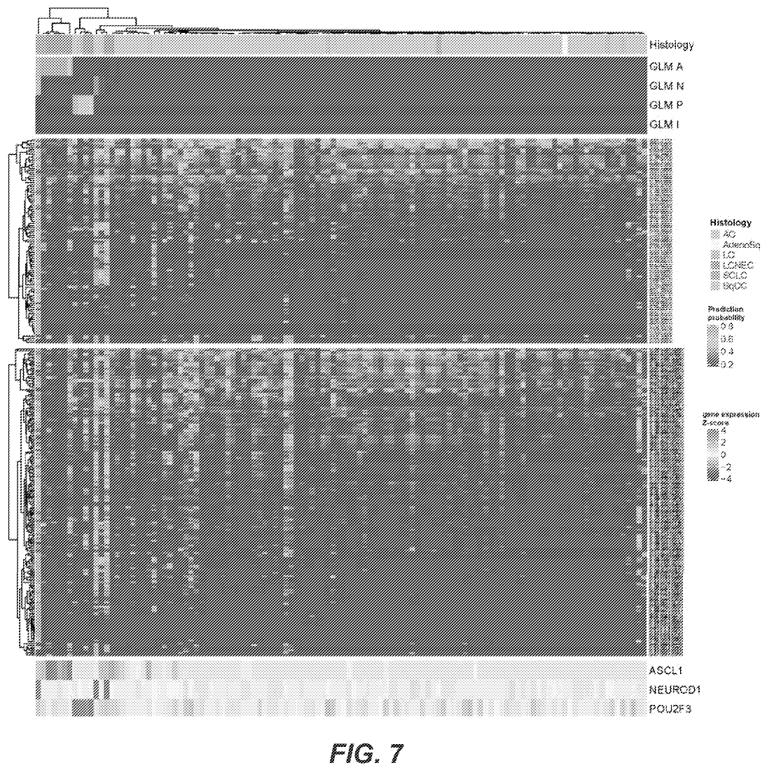

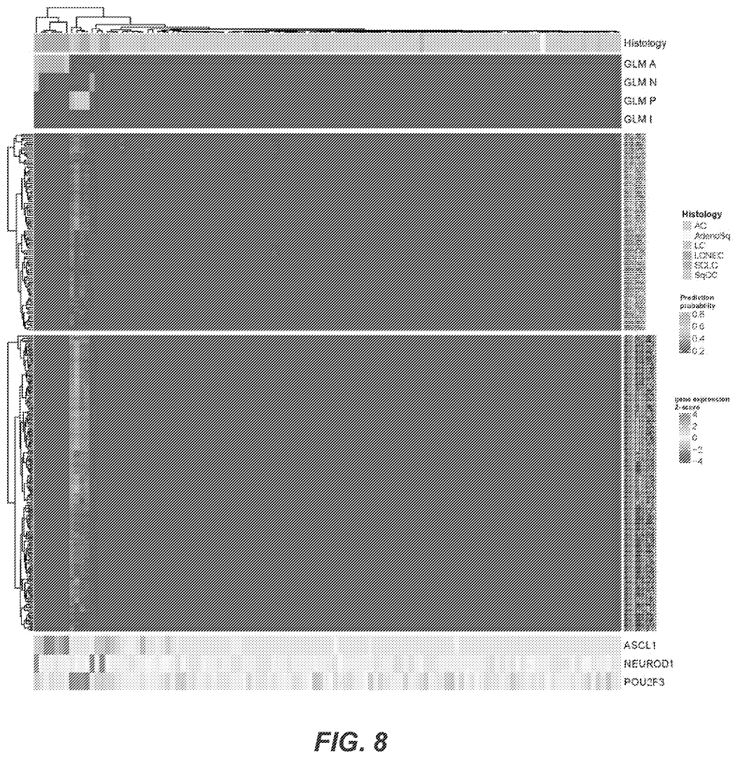

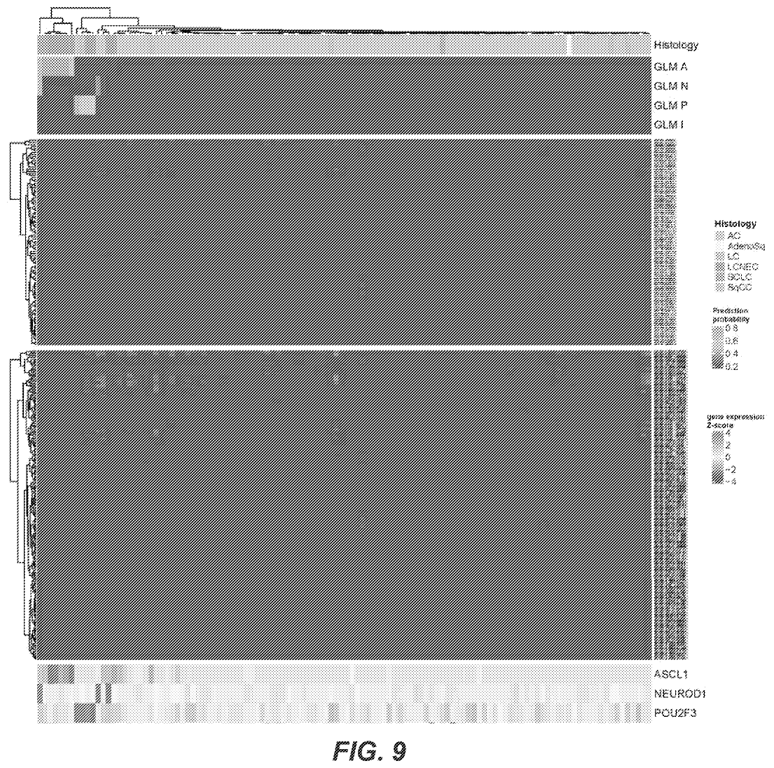

Importantly, the models were trained using 15,000 methylation markers per subtype. To analyze whether the reduction to 2-3 markers was feasible, the inventors generated models using only the reduced marker-set by assessing all combinations of the top markers that were selected for further analysis in plasma (Table 6). The results are highlighted in FIG. 6 (SCLC-A), FIG. 7 (SCLC-N), FIG. 8 (SCLC-P), and FIG. 9 (SCLC-I). Importantly, the reduced models using only 2-3 markers aligned well with the expression of the markers and showed concordant results with the larger models incorporating 15.000 methylation sites. This clearly demonstrated that reducing the markers was a feasible approach.

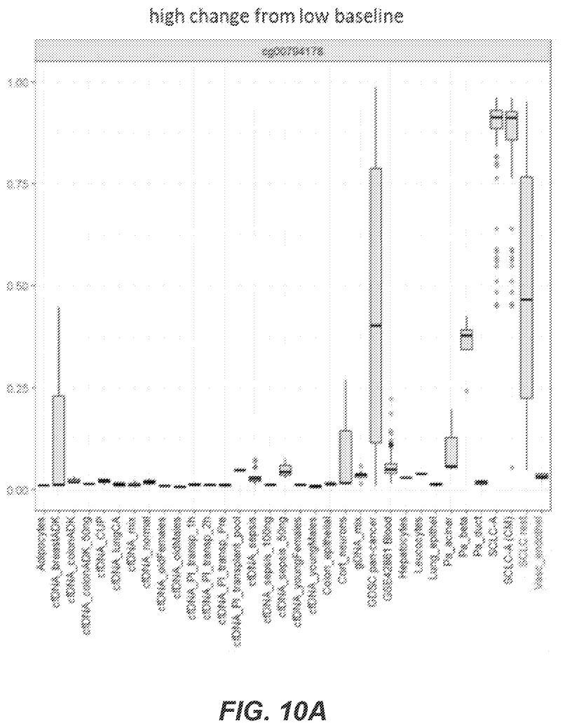

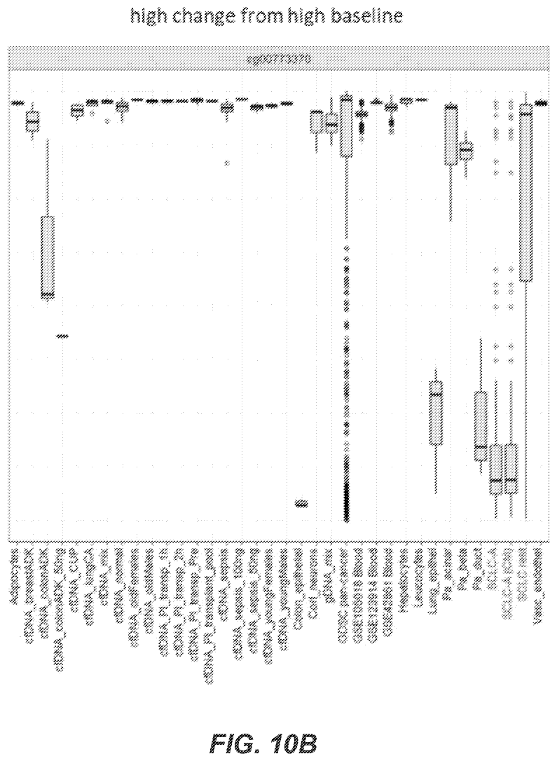

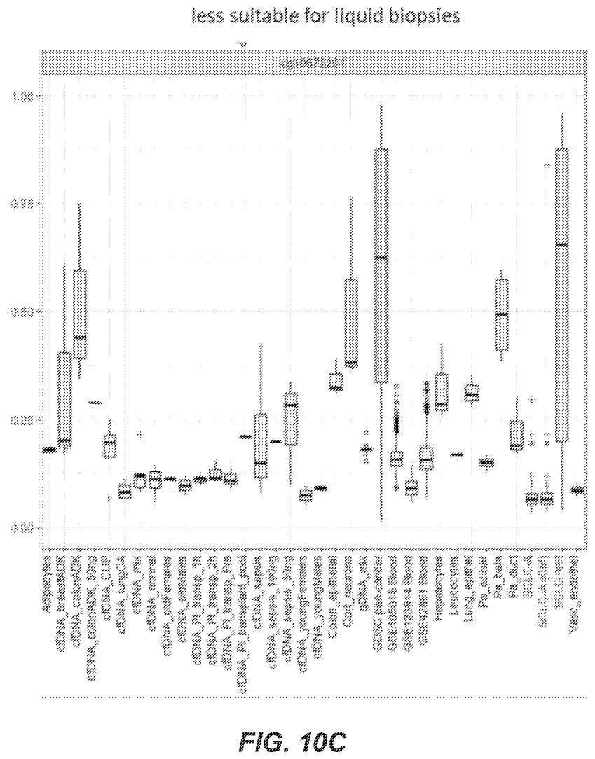

All methylation sites were assessed for their suitability in a liquid biopsy assay. Therefore, several resources comprising of DNA methylation from blood samples (GSE15018, GSE42861, GSE123914) and from various tissue including ctDNA (cfDNA Atlas Project). Methylation sites with a large difference from background methylation (defined by the various sources) were defined to be suitable in a liquid biopsy assay. Some examples are given in FIG. 10. Methylation sites suitable for analysis using a liquid biopsy assay are shown in Tables 6, 7, 8, 9, and 10.

TABLE 1
Top 1000 methylation sites associated with each of the SCLC-A, -N, -P, and -I subtypes

| Methylation | Position | | Associated | | | Relation to |
|---|---|---|---|---|---|---|
| Site | (GRCh 37) | Gene | Subgroup | Strand | CpG Island Name | CpG Island |
| cg20728514 | chr22: 44568423 | PARVG.TSS1500 | SCLC-A | R | | |
| cg13429423 | chr22: 44568775 | PARVG.TSS200 | SCLC-A | F | | |
| cg04506569 | chr2: 234837977 | TRPM8.GENEBODY | SCLC-A | F | | |
| cg17542751 | chr22: 44568725 | PARVG.TSS200 | SCLC-A | R | | |
| cg12688942 | chr1: 19680353 | CAPZB.GENEBODY | SCLC-A | R | | |
| cg25353896 | chr22: 44568753 | PARVG.TSS200 | SCLC-A | F | | |
| cg02911509 | chr22: 44568770 | PARVG.TSS200 | SCLC-A | F | | |
| cg16873414 | chr22: 44568699 | PARVG.TSS200 | SCLC-A | R | | |
| cg25901559 | chr7: 157585255 | PTPRN2.GENEBODY | SCLC-A | F | chr7: 157586711- | N_Shore |
| | | | | | 157587112 | |
| cg07639982 | chr10: 12555079 | CAMK1D.GENEBODY | SCLC-A | F | | |
| cg08271031 | chr22: 44568812 | PARVG.TSS200 | SCLC-A | R | | |
| cg17622855 | chr6: 157982358 | ZDHHC14.GENEBODY | SCLC-A | R | | |
| cg03817675 | chr7: 139299053 | HIPK2.GENEBODY | SCLC-A | R | | |
| cg17252884 | chr3: 15369681 | SH3BP5.GENEBODY | SCLC-A | F | chr3: 15373314- | N_Shelf |
| | | | | | 15374209 | |
| cg07213482 | chr4: 89660237 | FAM13A.GENEBODY | SCLC-A | R | | |
| cg12907969 | chr5: 89815060 | LYSMD3.GENEBODY | SCLC-A | R | | |
| cg25694807 | chr2: 161329607 | RBMS1.GENEBODY | SCLC-A | F | | |
| cg00090674 | chr22: 50446678 | IL17REL.UTR5 | SCLC-A | F | chr22: 50448567- | N_Shore |
| | | | | | 50449521 | |
| cg23317857 | chr6: 41696297 | TFEB.UTR5 | SCLC-A | F | | |
| cg11201256 | chr1: 184403226 | C1orf21.UTR5 | SCLC-A | R | | |
| cg16860004 | chr8: 103137229 | NCALD.TSS1500 | SCLC-A | F | chr8: 103135914- | S_Shore |
| | | | | | 103136775 | |
| cg15892280 | chr21: 40180000 | ETS2.UTR5 | SCLC-A | F | chr21: 40177002- | S_Shore |
| | | | | | 40178667 | |
| cg03568507 | chr2: 240153791 | HDAC4.GENEBODY | SCLC-A | F | chr2: 240153476- | Island |
| | | | | | 240153792 | |
| cg19437757 | chr7: 157697057 | PTPRN2.GENEBODY | SCLC-A | R | chr7: 157695025- | S_Shore |
| | | | | | 157695358 | |
| cg27550325 | chr7: 157915584 | PTPRN2.GENEBODY | SCLC-A | R | chr7: 157918704- | N_Shelf |
| | | | | | 157919409 | |
| cg27122614 | chr7: 157913741 | PTPRN2.GENEBODY | SCLC-A | R | | |
| cg27110054 | chr8: 103137274 | NCALD.TSS1500 | SCLC-A | R | chr8: 103135914- | S_Shore |
| | | | | | 103136775 | |
| cg07572930 | chr16: 81527107 | CMIP.GENEBODY | SCLC-A | F | | |
| cg17479501 | chr17: 80806191 | TBCD.GENEBODY | SCLC-A | R | | |
| cg02161503 | chr7: 157955692 | PTPRN2.GENEBODY | SCLC-A | R | chr7: 157956911- | N_Shore |
| | | | | | 157957273 | |
| cg13287553 | chr7: 4784419 | FOXK1.GENEBODY | SCLC-A | F | chr7: 4784820-4785058 | N_Shore |
| cg25746499 | chr2: 240153778 | HDAC4.GENEBODY | SCLC-A | F | chr2: 240153476- | Island |
| | | | | | 240153792 | |
| cg10672201 | chr12: 103353320 | ASCL1.UTR3 | SCLC-A | F | chr12: 103351579- | S_Shore |
| | | | | | 103352695 | |
| cg06043710 | chr10: 560371 | DIP2C.GENEBODY | SCLC-A | F | | |
| cg18966819 | chr7: 4784325 | FOXK1.GENEBODY | SCLC-A | R | chr7: 4784820-4785058 | N_Shore |
| cg18578954 | chr6: 28887247 | TRIM27.GENEBODY | SCLC-A | F | chr6: 28890951- | N_Shelf |
| | | | | | 28892013 | |
| cg06228260 | chr7: 157854729 | PTPRN2.GENEBODY | SCLC-A | R | | |
| cg03899215 | chr7: 157996423 | PTPRN2.GENEBODY | SCLC-A | R | chr7: 157996642- | N_Shore |
| | | | | | 157997027 | |
| cg08946713 | chr2: 191844998 | STAT1.GENEBODY | SCLC-A | F | | |
| cg18424850 | chr10: 132945786 | TCERG1L.GENEBODY | SCLC-A | R | | |
| cg21167502 | chr14: 104195666 | ZFYVE21.GENEBODY | SCLC-A | R | chr14: 104193901- | S_Shore |
| | | | | | 104194627 | |
| cg27119612 | chr15: 57491001 | TCF12.GENEBODY | SCLC-A | R | | |
| cg04921578 | chr13: 101143257 | PCCA.GENEBODY | SCLC-A | F | | |
| cg08277369 | chr16: 84538885 | KIAA1609.TSS1500 | SCLC-A | F | chr16: 84538884- | Island |
| | | | | | 84539115 | |
| cg08448341 | chr6: 134324873 | SLC2A12.GENEBODY | SCLC-A | F | | |
| cg15193475 | chr15: 80998711 | FAM108C1.GENEBODY | SCLC-A | F | | |
| cg17468616 | chr1: 226013212 | EPHX1.EXON1 | SCLC-A | F | | |
| cg18730511 | chr22: 50435413 | IL17REL.UTR3 | SCLC-A | F | chr22: 50438257- | N_Shelf |
| | | | | | 50438776 | |
| cg26239495 | chr11: 122791447 | C11orf63.GENEBODY | SCLC-A | R | | |
| cg01441025 | chr8: 103136942 | NCALD.EXON1 | SCLC-A | F | chr8: 103135914- | S_Shore |
| | | | | | 103136775 | |
| cg15577272 | chr7: 157932397 | PTPRN2.GENEBODY | SCLC-A | F | | |
| cg21373996 | chr10: 560669 | DIP2C.GENEBODY | SCLC-A | R | | |
| cg06650067 | chr15: 66084924 | DENND4A.TSS1500 | SCLC-A | R | chr15: 66083839- | Island |
| | | | | | 66084925 | |
| cg08951403 | chr7: 157960420 | PTPRN2.GENEBODY | SCLC-A | F | chr7: 157956911- | S_Shelf |
| | | | | | 157957273 | |
| cg09500207 | chr11: 68249304 | SAPS3.UTR5 | SCLC-A | R | | |
| cg16896647 | chr9: 93563776 | SYK.TSS1500 | SCLC-A | R | chr9: 93563775- | Island |
| | | | | | 93564546 | |
| cg10227678 | chr16: 54096340 | FTO.GENEBODY | SCLC-A | F | | |
| cg18883682 | chr7: 36655667 | AOAH.GENEBODY | SCLC-A | R | | |
| cg03558805 | chr7: 73184883 | CLDN3.TSS1500 | SCLC-A | F | chr7: 73183379- | Island |
| | | | | | 73185115 | |
| cg03856153 | chr3: 61904580 | PTPRG.GENEBODY | SCLC-A | R | | |
| cg04610718 | chr12: 103353362 | ASCL1.UTR3 | SCLC-A | F | chr12: 103351579- | S_Shore |
| | | | | | 103352695 | |
| cg06946543 | chr13: 76377366 | LMO7.UTR5 | SCLC-A | F | | |
| cg09845489 | chr7: 157667853 | PTPRN2.GENEBODY | SCLC-A | R | chr7: 157666069- | S_Shore |
| | | | | | 157666399 | |
| cg10919624 | chr7: 158279891 | PTPRN2.GENEBODY | SCLC-A | F | chr7: 158281373- | N_Shore |
| | | | | | 158281734 | |
| cg00799539 | chr12: 103353183 | ASCL1.UTR3 | SCLC-A | R | chr12: 103351579- | S_Shore |
| | | | | | 103352695 | |
| cg06663149 | chr4: 41457558 | LIMCH1.GENEBODY | SCLC-A | F | | |
| cg13656878 | chr9: 119048807 | PAPPA.GENEBODY | SCLC-A | F | | |
| cg14207833 | chr19: 18945153 | UPF1.GENEBODY | SCLC-A | F | chr19: 18942432- | S_Shelf |
| | | | | | 18943127 | |
| cg16762386 | chr1: 27240514 | NROB2.EXON1 | SCLC-A | F | | |
| cg26350286 | chr8: 95652691 | ESRP1.TSS1500 | SCLC-A | R | chr8: 95652455- | Island |
| | | | | | 95652873 | |
| cg00624799 | chr15: 90605618 | ZNF710.UTR5 | SCLC-A | R | | |
| cg03519907 | chr5: 154092426 | LARP1.TSS200 | SCLC-A | F | | |
| cg09176199 | chr1: 246383021 | SMYD3.GENEBODY | SCLC-A | F | | |
| cg15192120 | chr7: 157626176 | PTPRN2.GENEBODY | SCLC-A | R | | |
| cg16048383 | chr12: 6483708 | SCNN1A.GENEBODY | SCLC-A | R | | |
| cg22550299 | chr6: 169654842 | THBS2.TSS1500 | SCLC-A | R | | |
| cg23936766 | chr1: 230406371 | GALNT2.GENEBODY | SCLC-A | R | | |
| cg02390801 | chr15: 74528565 | CCDC33.TSS200 | SCLC-A | F | | |
| cg05503433 | chr19: 42409117 | ARHGEF1.GENEBODY | SCLC-A | F | chr19: 42408315- | S_Shore |
| | | | | | 42408535 | |
| cg09259081 | chr16: 84538889 | KIAA1609.TSS1500 | SCLC-A | F | chr16: 84538884- | Island |
| | | | | | 84539115 | |
| cg13547053 | chr2: 179184864 | OSBPL6.TSS200 | SCLC-A | R | | |
| cg25037165 | chr11: 12824283 | TEAD1.GENEBODY | SCLC-A | R | | |
| cg01187496 | chr5: 167718124 | WWC1.TSS1500 | SCLC-A | F | chr5: 167718523- | N_Shore |
| | | | | | 167719688 | |
| cg06338523 | chr7: 158279818 | PTPRN2.GENEBODY | SCLC-A | F | chr7: 158281373- | N_Shore |
| | | | | | 158281734 | |
| cg14947787 | chr14: 65405931 | GPX2.UTR3 | SCLC-A | F | | |
| cg16311186 | chr7: 140114137 | RAB19.GENEBODY | SCLC-A | F | | |
| cg17176609 | chr2: 74135217 | ACTG2.GENEBODY | SCLC-A | R | | |
| cg19648306 | chr2: 179184854 | OSBPL6.TSS200 | SCLC-A | R | | |
| cg19747960 | chr8: 11269117 | C8orf12.GENEBODY | SCLC-A | R | | |
| cg21867891 | chr18: 56529835 | ZNF532.TSS1500 | SCLC-A | R | chr18: 56530395- | N_Shore |
| | | | | | 56531288 | |
| cg07124138 | chr16: 88683903 | ZC3H18.GENEBODY | SCLC-A | R | | |
| cg27336068 | chr10: 1336103 | ADARB2.GENEBODY | SCLC-A | R | | |
| cg19954227 | chr7: 158113303 | PTPRN2.GENEBODY | SCLC-A | F | chr7: 158109334- | S_Shelf |
| | | | | | 158109594 | |
| cg20802509 | chr1: 229764652 | URB2.GENEBODY | SCLC-A | F | chr1: 229761128- | S_Shelf |
| | | | | | 229762299 | |
| cg21679730 | chr1: 65047485 | CACHD1.GENEBODY | SCLC-A | R | | |
| cg21732383 | chr6: 169654692 | THBS2.TSS1500 | SCLC-A | F | | |
| cg02428178 | chr15: 49104237 | CEP152.TSS1500 | SCLC-A | R | chr15: 49102731- | S_Shore |
| | | | | | 49102938 | |
| cg09450352 | chr7: 157495481 | PTPRN2.GENEBODY | SCLC-A | R | chr7: 157494510- | S_Shore |
| | | | | | 157494739 | |
| cg13579752 | chr5: 153577937 | GALNT10.GENEBODY | SCLC-A | F | | |
| cg14647989 | chr22: 43824766 | MPPED1.GENEBODY | SCLC-A | R | chr22: 43820887- | S_Shelf |
| | | | | | 43821136 | |
| cg14667175 | chr2: 111623300 | ACOXL.GENEBODY | SCLC-A | F | | |
| cg19212465 | chr7: 157654170 | PTPRN2.GENEBODY | SCLC-A | F | | |
| cg25029446 | chr7: 157495282 | PTPRN2.GENEBODY | SCLC-A | F | chr7: 157494510- | S_Shore |
| | | | | | 157494739 | |
| cg00064840 | chr7: 4784485 | FOXK1.GENEBODY | SCLC-A | F | chr7: 4784820-4785058 | N_Shore |
| cg02473460 | chr8: 93074521 | RUNX1T1.GENEBODY | SCLC-A | R | | |
| cg10880973 | chr6: 157982213 | ZDHHC14.GENEBODY | SCLC-A | R | | |
| cg15046304 | chr17: 75205489 | SEC14L1.GENEBODY | SCLC-A | F | chr17: 75209459- | N_Shelf |
| | | | | | 75209672 | |
| cg22598247 | chr7: 157614399 | PTPRN2.GENEBODY | SCLC-A | R | chr7: 157612036- | S_Shelf |
| | | | | | 157612316 | |
| cg26247373 | chr12: 57655696 | R3HDM2.GENEBODY | SCLC-A | F | | |
| cg00911446 | chr1: 7520079 | CAMTA1.GENEBODY | SCLC-A | F | | |
| cg12761282 | chr20: 2276571 | TGM3.TSS200 | SCLC-A | R | | |
| cg16645907 | chr5: 154092360 | LARP1.TSS200 | SCLC-A | F | | |
| cg22057874 | chr2: 179184866 | OSBPL6.TSS200 | SCLC-A | R | | |
| cg24928687 | chr1: 226012913 | EPHX1.TSS200 | SCLC-A | R | | |
| cg00668890 | chr12: 6449990 | TNFRSF1A.GENEBODY | SCLC-A | R | | |
| cg02901644 | chr2: 234369787 | DGKD.GENEBODY | SCLC-A | R | chr2: 234369786- | Island |
| | | | | | 234370099 | |
| cg07696006 | chr15: 74528563 | CCDC33.TSS200 | SCLC-A | F | | |
| cg11575295 | chr7: 158250911 | PTPRN2.GENEBODY | SCLC-A | F | chr7: 158247888- | S_Shore |
| | | | | | 158249312 | |
| cg15228639 | chr15: 42289939 | PLA2G4E.GENEBODY | SCLC-A | R | | |
| cg24241530 | chr7: 158061820 | PTPRN2.GENEBODY | SCLC-A | R | chr7: 158060894- | S_Shore |
| | | | | | 158061100 | |
| cg27555529 | chr19: 13617518 | CACNA1A.TSS1500 | SCLC-A | F | chr19: 13616752- | S_Shore |
| | | | | | 13617267 | |
| cg08758185 | chr5: 150849936 | SLC36A1.GENEBODY | SCLC-A | F | | |
| cg10899175 | chr2: 11680144 | GREB1.EXON1 | SCLC-A | F | | |
| cg15298059 | chr7: 157668141 | PTPRN2.GENEBODY | SCLC-A | F | chr7: 157669879- | N_Shore |
| | | | | | 157670095 | |
| cg18635670 | chr6: 26539327 | HMGN4.UTR5 | SCLC-A | R | chr6: 26538496- | S_Shore |
| | | | | | 26539015 | |
| cg20149780 | chr1: 15751063 | EFHD2.GENEBODY | SCLC-A | F | | |
| cg03877767 | chr2: 11680057 | GREB1.UTR5 | SCLC-A | F | | |
| cg10126234 | chr14: 104195533 | ZFYVE21.GENEBODY | SCLC-A | R | chr14: 104193901- | S_Shore |
| | | | | | 104194627 | |
| cg11035519 | chr11: 130051458 | ST14.GENEBODY | SCLC-A | F | | |
| cg11281961 | chr2: 97498902 | CNNM3.UTR3 | SCLC-A | R | | |
| cg12098949 | chr17: 75367442 | SEPT9.UTR5 | SCLC-A | F | chr17: 75368688- | N_Shore |
| | | | | | 75370506 | |
| cg18738906 | chr12: 6483739 | SCNN1A.GENEBODY | SCLC-A | R | | |
| cg24034277 | chr14: 102467421 | DYNC1H1.GENEBODY | SCLC-A | F | | |
| cg25264630 | chr7: 157849744 | PTPRN2.GENEBODY | SCLC-A | F | | |
| cg26849830 | chr12: 109178373 | SSH1.UTR3 | SCLC-A | R | | |
| cg06221087 | chr1: 164659666 | PBX1.GENEBODY | SCLC-A | F | | |
| cg06694040 | chr11: 11301699 | GALNTL4.GENEBODY | SCLC-A | F | | |
| cg07875069 | chr7: 129985025 | CPA5.EXON1 | SCLC-A | R | | |
| cg19308132 | chr8: 103137173 | NCALD.TSS1500 | SCLC-A | F | chr8: 103135914- | S_Shore |
| | | | | | 103136775 | |
| cg19897017 | chr7: 2163380 | MAD1L1.GENEBODY | SCLC-A | F | | |
| cg27655671 | chr6: 38207741 | BTBD9.GENEBODY | SCLC-A | F | | |
| cg05800761 | chr18: 56529852 | ZNF532.TSS1500 | SCLC-A | F | chr18: 56530395- | N_Shore |
| | | | | | 56531288 | |
| cg20371765 | chr7: 8302271 | ICA1.TSS200 | SCLC-A | F | chr7: 8301031-8302252 | S_Shore |
| cg02716952 | chr7: 148504681 | EZH2.UTR3 | SCLC-A | R | | |
| cg14592260 | chr11: 108798655 | DDX10.GENEBODY | SCLC-A | F | | |
| cg16937735 | chr7: 157562675 | PTPRN2.GENEBODY | SCLC-A | R | chr7: 157560974- | S_Shore |
| | | | | | 157561195 | |
| cg17208060 | chr2: 11680086 | GREB1.EXON1 | SCLC-A | R | | |
| cg22968401 | chr17: 27370009 | PIPOX.UTR5 | SCLC-A | F | | |
| cg23884690 | chr13: 113678926 | MCF2L.GENEBODY | SCLC-A | R | chr13: 113676781- | S_Shore |
| | | | | | 113677120 | |
| cg27431545 | chr2: 173607996 | RAPGEF4.GENEBODY | SCLC-A | F | | |
| cg00283887 | chr7: 157935557 | PTPRN2.GENEBODY | SCLC-A | F | chr7: 157937614- | N_Shelf |
| | | | | | 157937841 | |
| cg00773370 | chr12: 6483772 | SCNN1A.GENEBODY | SCLC-A | R | | |
| cg02842704 | chr5: 151305746 | GLRA1.TSS1500 | SCLC-A | R | chr5: 151304226- | S_Shore |
| | | | | | 151304824 | |
| cg02865101 | chr7: 157779894 | PTPRN2.GENEBODY | SCLC-A | R | | |
| cg05579861 | chr7: 157631040 | PTPRN2.GENEBODY | SCLC-A | F | chr7: 157631319- | N_Shore |
| | | | | | 157631615 | |
| cg06650260 | chr1: 27240478 | NROB2.EXON1 | SCLC-A | R | | |
| cg07685977 | chr10: 133036507 | TCERG1L.GENEBODY | SCLC-A | F | | |
| cg13213923 | chr17: 60885491 | MARCH10.UTR5 | SCLC-A | R | chr17: 60885408- | Island |
| | | | | | 60885857 | |
| cg19809865 | chr14: 44976233 | FSCB.EXON1 | SCLC-A | R | | |
| cg20986887 | chr6: 28887284 | TRIM27.GENEBODY | SCLC-A | R | chr6: 28890951- | N_Shelf |
| | | | | | 28892013 | |
| cg23245485 | chr12: 6484219 | SCNN1A.UTR5 | SCLC-A | R | | |
| cg24085344 | chr17: 47485676 | PHB.GENEBODY | SCLC-A | R | | |
| cg25148025 | chr1: 164681267 | PBX1.GENEBODY | SCLC-A | F | | |
| cg00787726 | chr10: 86113284 | FAM190B.UTR5 | SCLC-A | F | | |
| cg03900492 | chr7: 157345659 | PTPRN2.GENEBODY | SCLC-A | R | chr7: 157346613- | N_Shore |
| | | | | | 157347069 | |
| cg04892919 | chr2: 191272604 | MFSD6.TSS1500 | SCLC-A | R | chr2: 191272484- | Island |
| | | | | | 191273671 | |
| cg07942479 | chr4: 185747981 | ACSL1.TSS1500 | SCLC-A | R | chr4: 185746557- | S_Shore |
| | | | | | 185747827 | |
| cg00448761 | chr19: 6066102 | RFX2.UTR5 | SCLC-A | R | | |
| cg01792117 | chr17: 27088227 | C17orf63.GENEBODY | SCLC-A | F | | |
| cg02567323 | chr2: 48671699 | KLRAQ1.GENEBODY | SCLC-A | R | chr2: 48667559- | S_Shelf |
| | | | | | 48668355 | |
| cg08932665 | chr11: 71937515 | INPPL1.GENEBODY | SCLC-A | F | chr11: 71934247- | S_Shore |
| | | | | | 71936321 | |
| cg21380084 | chr7: 157937891 | PTPRN2.GENEBODY | SCLC-A | R | chr7: 157937614- | S_Shore |
| | | | | | 157937841 | |
| cg23632237 | chr20: 30560483 | XKR7.GENEBODY | SCLC-A | F | chr20: 30555901- | S_Shelf |
| | | | | | 30556502 | |
| cg25401010 | chr18: 56529878 | ZNF532.TSS200 | SCLC-A | F | chr18: 56530395- | N_Shore |
| | | | | | 56531288 | |
| cg26312133 | chr17: 71428204 | SDK2.GENEBODY | SCLC-A | F | chr17: 71431621- | N_Shelf |
| | | | | | 71431878 | |
| cg01321816 | chr15: 91358514 | BLM.UTR3 | SCLC-A | R | chr15: 91362445- | N_Shelf |
| | | | | | 91362658 | |
| cg03653377 | chr7: 158107573 | PTPRN2.GENEBODY | SCLC-A | F | chr7: 158109334- | N_Shore |
| | | | | | 158109594 | |
| cg24216770 | chr20: 35091947 | DLGAP4.GENEBODY | SCLC-A | F | chr20: 35089448- | S_Shore |
| | | | | | 35089998 | |
| cg25766748 | chr19: 13614882 | CACNA1A.GENEBODY | SCLC-A | F | chr19: 13616752- | N_Shore |
| | | | | | 13617267 | |
| cg26639747 | chr7: 157590318 | PTPRN2.GENEBODY | SCLC-A | R | chr7: 157593887- | N_Shelf |
| | | | | | 157594128 | |
| cg27388763 | chr17: 44648385 | ARL17A.GENEBODY | SCLC-A | R | | |
| cg00362322 | chr16: 88704230 | IL17C.TSS1500 | SCLC-A | F | chr16: 88706211- | N_Shore |
| | | | | | 88706492 | |
| cg04874261 | chr3: 71773745 | EIF4E3.GENEBODY | SCLC-A | F | chr3: 71773826- | N_Shore |
| | | | | | 71774873 | |
| cg09130676 | chr7: 158220856 | PTPRN2.GENEBODY | SCLC-A | R | chr7: 158217851- | S_Shore |
| | | | | | 158220709 | |
| cg18011401 | chr19: 13617366 | CACNA1A.TSS200 | SCLC-A | F | chr19: 13616752- | S_Shore |
| | | | | | 13617267 | |
| cg19307406 | chr1: 167874258 | ADCY10.UTR5 | SCLC-A | R | | |
| cg19404354 | chr1: 202093479 | GPR37L1.GENEBODY | SCLC-A | F | | |
| cg19637113 | chr15: 74528522 | CCDC33.TSS200 | SCLC-A | F | | |
| cg19726712 | chr8: 98790327 | LAPTM4B.GENEBODY | SCLC-A | F | chr8: 98787835- | S_Shore |
| | | | | | 98788874 | |
| cg23969274 | chr6: 116851736 | FAM26D.UTR5 | SCLC-A | R | | |
| cg13024807 | chr6: 53152718 | ELOVL5.GENEBODY | SCLC-A | R | | |
| cg01100465 | chr7: 157585095 | PTPRN2.GENEBODY | SCLC-A | F | chr7: 157586711- | N_Shore |
| | | | | | 157587112 | |
| cg04373993 | chr7: 157570916 | PTPRN2.GENEBODY | SCLC-A | F | chr7: 157568163- | S_Shelf |
| | | | | | 157568404 | |
| cg05046377 | chr11: 4927915 | OR51A7.TSS1500 | SCLC-A | F | | |
| cg06160809 | chr2: 217559688 | IGFBP5.UTR5 | SCLC-A | F | chr2: 217559039- | S_Shore |
| | | | | | 217559484 | |
| cg06676049 | chr2: 23839842 | KLHL29.GENEBODY | SCLC-A | R | | |
| cg06912665 | chr5: 154092290 | LARP1.TSS200 | SCLC-A | R | | |
| cg07927098 | chr7: 157866704 | PTPRN2.GENEBODY | SCLC-A | R | | |
| cg09644722 | chr17: 60885519 | MARCH10.UTR5 | SCLC-A | R | chr17: 60885408- | Island |
| | | | | | 60885857 | |
| cg15726025 | chr1: 32681995 | C1orf91.UTR3 | SCLC-A | F | | |
| cg19188359 | chr20: 2312099 | TGM3.GENEBODY | SCLC-A | R | chr20: 2308778-2309042 | S_Shelf |
| cg19890739 | chr16: 85723539 | GINS2.TSS1500 | SCLC-A | R | chr16: 85722209- | S_Shore |
| | | | | | 85722941 | |
| cg23309969 | chr18: 56529866 | ZNF532.TSS200 | SCLC-A | F | chr18: 56530395- | N_Shore |
| | | | | | 56531288 | |
| cg25507001 | chr5: 137475288 | NME5.TSS200 | SCLC-A | R | | |
| cg26546155 | chr16: 57269635 | RSPRY1.GENEBODY | SCLC-A | F | | |
| cg01520105 | chr6: 111887243 | TRAF3IP2.GENEBODY | SCLC-A | R | | |
| cg04911819 | chr10: 24504859 | KIAA1217.GENEBODY | SCLC-A | F | | |
| cg08471739 | chr3: 129312902 | PLXND1.GENEBODY | SCLC-A | F | | |
| cg15120341 | chr13: 114238565 | TFDP1.TSS1500 | SCLC-A | F | | |
| cg01277983 | chr22: 43835600 | MPPED1.GENEBODY | SCLC-A | F | | |
| cg01942646 | chr1: 27240694 | NROB2.TSS200 | SCLC-A | R | | |
| cg02639667 | chr5: 154092321 | LARP1.TSS200 | SCLC-A | F | | |
| cg08353938 | chr2: 217227472 | MARCH4.GENEBODY | SCLC-A | R | | |
| cg26912251 | chr6: 168970824 | SMOC2.GENEBODY | SCLC-A | R | chr6: 168972516- | N_Shore |
| | | | | | 168974109 | |
| cg03308120 | chr7: 157933784 | PTPRN2.GENEBODY | SCLC-A | F | chr7: 157937614- | N_Shelf |
| | | | | | 157937841 | |
| cg07015608 | chr7: 158114303 | PTPRN2.GENEBODY | SCLC-A | R | chr7: 158110569- | S_Shelf |
| | | | | | 158110881 | |
| cg08698159 | chr17: 27294743 | SEZ6.GENEBODY | SCLC-A | R | | |
| cg08725319 | chr7: 157334751 | PTPRN2.GENEBODY | SCLC-A | R | | |
| cg13531977 | chr9: 112013420 | EPB41L4B.GENEBODY | SCLC-A | R | | |
| cg19779605 | chr11: 4703553 | OR51E2.GENEBODY | SCLC-A | R | | |
| cg20448594 | chr7: 73940352 | GTF2IRD1.GENEBODY | SCLC-A | R | | |
| cg21857959 | chr3: 182896995 | MCF2L2.UTR3 | SCLC-A | F | chr3: 182896920- | Island |
| | | | | | 182897381 | |
| cg26572499 | chr15: 22929910 | CYFIP1.GENEBODY | SCLC-A | R | | |
| cg04198210 | chr15: 74528606 | CCDC33.TSS200 | SCLC-A | F | | |
| cg05204104 | chr2: 235403141 | ARL4C.UTR3 | SCLC-A | R | chr2: 235404502- | N_Shore |
| | | | | | 235406541 | |
| cg05822926 | chr7: 8302235 | ICA1.TSS200 | SCLC-A | F | chr7: 8301031-8302252 | Island |
| cg18360873 | chr10: 133051669 | TCERG1L.GENEBODY | SCLC-A | F | | |
| cg00610577 | chr12: 12008666 | ETV6.GENEBODY | SCLC-A | R | | |
| cg07675656 | chr16: 2105015 | TSC2.GENEBODY | SCLC-A | R | chr16: 2105014-2105390 | Island |
| cg11812748 | chr1: 246733057 | CNST.UTR5 | SCLC-A | F | chr1: 246729207- | S_Shelf |
| | | | | | 246730276 | |
| cg14178173 | chr7: 157495532 | PTPRN2.GENEBODY | SCLC-A | F | chr7: 157494510- | S_Shore |
| | | | | | 157494739 | |
| cg20724810 | chr3: 182897196 | MCF2L2.GENEBODY | SCLC-A | R | chr3: 182896920- | Island |
| | | | | | 182897381 | |
| cg21831937 | chr15: 57519802 | TCF12.GENEBODY | SCLC-A | F | | |
| cg22049038 | chr7: 146332766 | CNTNAP2.GENEBODY | SCLC-A | R | | |
| cg23613317 | chr5: 154092109 | LARP1.TSS1500 | SCLC-A | R | | |
| cg24580782 | chr1: 27240365 | NROB2.EXON1 | SCLC-A | R | | |
| cg25975382 | chr7: 157532236 | PTPRN2.GENEBODY | SCLC-A | F | | |
| cg01536803 | chr7: 157968578 | PTPRN2.GENEBODY | SCLC-A | F | | |
| cg06070324 | chr15: 75322058 | PPCDC.GENEBODY | SCLC-A | R | | |
| cg06413087 | chr7: 157929609 | PTPRN2.GENEBODY | SCLC-A | F | | |
| cg08203715 | chr11: 126226154 | ST3GAL4.EXON1 | SCLC-A | R | chr11: 126225355- | S_Shore |
| | | | | | 126226073 | |
| cg15067687 | chr7: 127658301 | SND1.GENEBODY | SCLC-A | F | | |
| cg15428620 | chr10: 102792835 | SFXN3.GENEBODY | SCLC-A | F | chr10: 102790820- | S_Shore |
| | | | | | 102791049 | |
| cg16681199 | chr6: 70990268 | COL9A1.GENEBODY | SCLC-A | R | chr6: 70992040- | N_Shore |
| | | | | | 70992912 | |
| cg17976873 | chr14: 23352295 | REM2.TSS200 | SCLC-A | F | chr14: 23355738- | N_Shelf |
| | | | | | 23356402 | |
| cg18717689 | chr9: 116971578 | MIR455.TSS200 | SCLC-A | R | | |
| cg25145687 | chr11: 1606664 | HCCA2.GENEBODY | SCLC-A | R | | |
| cg01154505 | chr2: 112940409 | FBLN7.GENEBODY | SCLC-A | F | chr2: 112939287- | S_Shore |
| | | | | | 112939563 | |
| cg03071245 | chr13: 37463034 | SMAD9.UTR5 | SCLC-A | F | | |
| cg03527077 | chr7: 158059704 | PTPRN2.GENEBODY | SCLC-A | R | chr7: 158060894- | N_Shore |
| | | | | | 158061100 | |
| cg07979652 | chr7: 157934005 | PTPRN2.GENEBODY | SCLC-A | R | chr7: 157937614- | N_Shelf |
| | | | | | 157937841 | |
| cg09409457 | chr7: 157954722 | PTPRN2.GENEBODY | SCLC-A | F | chr7: 157956911- | N_Shelf |
| | | | | | 157957273 | |
| cg13169065 | chr2: 166650947 | GALNT3.TSS200 | SCLC-A | F | chr2: 166649909- | Island |
| | | | | | 166650966 | |
| cg13619044 | chr7: 30202350 | C7orf41.UTR3 | SCLC-A | R | | |
| cg15707428 | chr2: 11679845 | GREB1.TSS1500 | SCLC-A | R | | |
| cg16287740 | chr7: 128595046 | TNPO3.UTR3 | SCLC-A | R | | |
| cg19343518 | chr6: 157508679 | ARID1B.GENEBODY | SCLC-A | F | | |
| cg20632462 | chr11: 20882850 | NELL1.GENEBODY | SCLC-A | F | | |
| cg24129115 | chr14: 23352488 | REM2.EXON1 | SCLC-A | F | chr14: 23355738- | N_Shelf |
| | | | | | 23356402 | |
| cg25439479 | chr8: 92971562 | RUNX1T1.UTR3 | SCLC-A | F | | |
| cg02873384 | chr11: 4936857 | OR51G2.EXON1 | SCLC-A | F | | |
| cg03892543 | chr16: 84650330 | COTL1.GENEBODY | SCLC-A | R | chr16: 84650960- | N_Shore |
| | | | | | 84651565 | |
| cg04466743 | chr20: 19915794 | RIN2.GENEBODY | SCLC-A | F | | |
| cg06424550 | chr10: 12808422 | CAMK1D.GENEBODY | SCLC-A | R | | |
| cg08886790 | chr2: 111857973 | ACOXL.GENEBODY | SCLC-A | LL | | |
| cg11959316 | chr1: 57001742 | PPAP2B.GENEBODY | SCLC-A | R | | |
| cg16396191 | chr13: 42184765 | KIAA0564.GENEBODY | SCLC-A | R | | |
| cg18352935 | chr11: 4843021 | OR51F2.EXON1 | SCLC-A | R | | |
| cg21185990 | chr7: 157835049 | PTPRN2.GENEBODY | SCLC-A | F | | |
| cg07056138 | chr8: 103120260 | NCALD.UTR5 | SCLC-A | F | | |
| cg07444408 | chr4: 141218466 | SCOC.UTR5 | SCLC-A | F | | |
| cg08871354 | chr16: 85723489 | GINS2.TSS1500 | SCLC-A | R | chr16: 85722209- | S_Shore |
| | | | | | 85722941 | |
| cg09027472 | chr7: 157981337 | PTPRN2.GENEBODY | SCLC-A | F | chr7: 157980786- | Island |
| | | | | | 157981462 | |
| cg11786338 | chr18: 56809717 | SEC11C.GENEBODY | SCLC-A | F | chr18: 56806608- | S_Shelf |
| | | | | | 56807425 | |
| cg14537549 | chr10: 12393185 | CAMK1D.GENEBODY | SCLC-A | R | chr10: 12390802- | S_Shore |
| | | | | | 12392589 | |
| cg16696476 | chr17: 8191344 | RANGRF.TSS1500 | SCLC-A | R | chr17: 8191864-8192495 | N_Shore |
| cg20239333 | chr10: 133111331 | TCERG1L.TSS1500 | SCLC-A | F | chr10: 133109193- | S_Shore |
| | | | | | 133111052 | |
| cg21088119 | chr8: 11251471 | C8orf12.GENEBODY | SCLC-A | F | | |
| cg23758354 | chr7: 157434590 | PTPRN2.GENEBODY | SCLC-A | R | | |
| cg27266257 | chr10: 5707799 | ASB13.GENEBODY | SCLC-A | F | chr10: 5707980-5708798 | N_Shore |
| cg03345925 | chr8: 144599347 | ZC3H3.GENEBODY | SCLC-A | F | chr8: 144601691- | N_Shelf |
| | | | | | 144601895 | |
| cg07998072 | chr16: 19425853 | TMC5.UTR5 | SCLC-A | F | chr16: 19421816- | S_Shelf |
| | | | | | 19422498 | |
| cg08917718 | chr3: 52002169 | PCBP4.TSS1500 | SCLC-A | R | | |
| cg09127540 | chr7: 157671453 | PTPRN2.GENEBODY | SCLC-A | R | chr7: 157669879- | S_Shore |
| | | | | | 157670095 | |
| cg10633463 | chr17: 79197669 | AZI1.TSS1500 | SCLC-A | R | chr17: 79196345- | S_Shore |
| | | | | | 79197419 | |
| cg11803278 | chr7: 157951799 | PTPRN2.GENEBODY | SCLC-A | F | chr7: 157951254- | Island |
| | | | | | 157951800 | |
| cg22797644 | chr6: 34206491 | HMGA1.UTR5 | SCLC-A | F | chr6: 34202567- | S_Shore |
| | | | | | 34206193 | |
| cg22994649 | chr7: 157641916 | PTPRN2.GENEBODY | SCLC-A | R | | |
| cg00572756 | chr7: 157863106 | PTPRN2.GENEBODY | SCLC-A | R | | |
| cg02079584 | chr15: 74528719 | CCDC33.EXON1 | SCLC-A | F | | |
| cg02111655 | chr10: 30338246 | KIAA1462.UTR5 | SCLC-A | R | | |
| cg03332546 | chr19: 16553887 | EPS15L1.GENEBODY | SCLC-A | F | | |
| cg03660606 | chr7: 157659983 | PTPRN2.GENEBODY | SCLC-A | F | chr7: 157659433- | Island |
| | | | | | 157660322 | |
| cg11857514 | chr12: 122151762 | TMEM120B.GENEBODY | SCLC-A | F | chr12: 122150513- | S_Shore |
| | | | | | 122151091 | |
| cg14037240 | chr10: 5707981 | ASB13.GENEBODY | SCLC-A | R | chr10: 5707980-5708798 | Island |
| cg17356036 | chr2: 240213057 | HDAC4.GENEBODY | SCLC-A | R | | |
| cg17985311 | chr1: 42801965 | FOXJ3.TSS1500 | SCLC-A | R | chr1: 42800265- | S_Shore |
| | | | | | 42801370 | |
| cg19126954 | chr7: 157616612 | PTPRN2.GENEBODY | SCLC-A | F | chr7: 157618856- | N_Shelf |
| | | | | | 157619302 | |
| cg25743482 | chr11: 19775505 | NAV2.GENEBODY | SCLC-A | F | | |
| cg02007867 | chr7: 157786536 | PTPRN2.GENEBODY | SCLC-A | R | | |
| cg02902261 | chr7: 72991150 | TBL2.GENEBODY | SCLC-A | R | chr7: 72992434- | N_Shore |
| | | | | | 72993397 | |
| cg06938143 | chr8: 11251609 | C8orf12.GENEBODY | SCLC-A | R | | |
| cg08850729 | chr1: 60036715 | FGGY.GENEBODY | SCLC-A | R | | |
| cg08989212 | chr21: 36096051 | NCRNA00160.TSS200 | SCLC-A | R | | |
| cg12193099 | chr6: 169654018 | THBS2.UTR5 | SCLC-A | R | | |
| cg12275410 | chr10: 135123239 | ZNF511.GENEBODY | SCLC-A | R | chr10: 135123238- | Island |
| | | | | | 135123448 | |
| cg12356107 | chr20: 22567037 | FOXA2.TSS1500 | SCLC-A | R | chr20: 22566821- | Island |
| | | | | | 22567055 | |
| cg15252509 | chr18: 56337165 | MALT1.TSS1500 | SCLC-A | R | chr18: 56338211- | N_Shore |
| | | | | | 56339277 | |
| cg16289848 | chr4: 79231477 | FRAS1.GENEBODY | SCLC-A | F | | |
| cg00827176 | chr8: 21885702 | NPM2.GENEBODY | SCLC-A | R | chr8: 21882245- | S_Shelf |
| | | | | | 21882883 | |
| cg01511231 | chr1: 1109676 | TTLL10.UTR5 | SCLC-A | F | chr1: 1109314-1110145 | Island |
| cg08202399 | chr16: 14280512 | MKL2.GENEBODY | SCLC-A | F | | |
| cg14122506 | chr9: 116060914 | RNF183.UTR5 | SCLC-A | R | | |
| cg16934969 | chr2: 173607848 | RAPGEF4.GENEBODY | SCLC-A | R | | |
| cg19755171 | chr17: 79197747 | AZI1.TSS1500 | SCLC-A | R | chr17: 79196345- | S_Shore |
| | | | | | 79197419 | |
| cg20649298 | chr20: 22545407 | C20orf56.GENEBODY | SCLC-A | R | chr20: 22548967- | N_Shelf |
| | | | | | 22549720 | |
| cg22420514 | chr7: 157997479 | PTPRN2.GENEBODY | SCLC-A | F | chr7: 157996642- | S_Shore |
| | | | | | 157997027 | |
| cg23997290 | chr20: 22402746 | LOC284788.TSS1500 | SCLC-A | F | | |
| cg27308682 | chr7: 157637500 | PTPRN2.GENEBODY | SCLC-A | F | | |
| cg01310118 | chr6: 168855168 | SMOC2.GENEBODY | SCLC-A | R | | |
| cg06155341 | chr11: 66048759 | CNIH2.GENEBODY | SCLC-A | R | chr11: 66049750- | N_Shore |
| | | | | | 66050229 | |
| cg07143635 | chr16: 11681487 | LITAF.TSS200 | SCLC-A | F | chr16: 11680008- | S_Shore |
| | | | | | 11681275 | |
| cg07551677 | chr8: 28570561 | EXTL3.UTR5 | SCLC-A | R | chr8: 28573597- | N_Shelf |
| | | | | | 28573840 | |
| cg13071618 | chr8: 144599125 | ZC3H3.GENEBODY | SCLC-A | R | chr8: 144601691- | N_Shelf |
| | | | | | 144601895 | |
| cg14706245 | chr7: 157536727 | PTPRN2.GENEBODY | SCLC-A | F | | |
| cg15175716 | chr11: 64120685 | CCDC88B.GENEBODY | SCLC-A | R | | |
| cg15504747 | chr3: 129312979 | PLXND1.GENEBODY | SCLC-A | R | | |
| cg20573218 | chr3: 181409863 | SOX2OT.GENEBODY | SCLC-A | F | chr3: 181413014- | N_Shelf |
| | | | | | 181414022 | |
| cg20825710 | chr11: 1620352 | HCCA2.GENEBODY | SCLC-A | R | | |
| cg23869743 | chr10: 99611477 | GOLGA7B.GENEBODY | SCLC-A | F | chr10: 99609425- | S_Shore |
| | | | | | 99610116 | |
| cg24119607 | chr5: 139047739 | CXXC5.UTR5 | SCLC-A | F | chr5: 139047905- | N_Shore |
| | | | | | 139048235 | |
| cg25809301 | chr3: 71179811 | FOXP1.GENEBODY | SCLC-A | F | | |
| cg00781208 | chr12: 64433590 | SRGAP1.GENEBODY | SCLC-A | R | | |
| cg01282269 | chr21: 41515306 | DSCAM.GENEBODY | SCLC-A | F | chr21: 41516237- | N_Shore |
| | | | | | 41516472 | |
| cg02098619 | chr1: 8689772 | RERE.GENEBODY | SCLC-A | R | | |
| cg02704103 | chr3: 72846991 | SHQ1.GENEBODY | SCLC-A | R | | |
| cg02704570 | chr7: 157647109 | PTPRN2.GENEBODY | SCLC-A | F | chr7: 157647800- | N_Shore |
| | | | | | 157648007 | |
| cg04188351 | chr11: 33744823 | CD59.UTR5 | SCLC-A | R | | |
| cg04545268 | chr7: 157653809 | PTPRN2.GENEBODY | SCLC-A | F | | |
| cg04583813 | chr10: 560323 | DIP2C.GENEBODY | SCLC-A | F | | |
| cg06287775 | chr7: 4784174 | FOXK1.GENEBODY | SCLC-A | R | chr7: 4784820-4785058 | N_Shore |
| cg06324554 | chr7: 157495841 | PTPRN2.GENEBODY | SCLC-A | R | chr7: 157494510- | S_Shore |
| | | | | | 157494739 | |
| cg07530194 | chr17: 78808450 | RPTOR.GENEBODY | SCLC-A | R | | |
| cg09215582 | chr4: 71575507 | RUFY3.GENEBODY | SCLC-A | R | chr4: 71570147- | S_Shelf |
| | | | | | 71571626 | |
| cg16177693 | chr1: 27240669 | NROB2.TSS200 | SCLC-A | R | | |
| cg18587271 | chr16: 46917496 | GPT2.TSS1500 | SCLC-A | F | chr16: 46917849- | N_Shore |
| | | | | | 46918957 | |
| cg19149693 | chr3: 114599007 | ZBTB20.UTR5 | SCLC-A | F | | |
| cg23694187 | chr7: 157335188 | PTPRN2.GENEBODY | SCLC-A | F | | |
| cg23861617 | chr4: 83422170 | TMEM150C.GENEBODY | SCLC-A | F | | |
| cg00909926 | chr1: 216774208 | ESRRG.GENEBODY | SCLC-A | R | chr1: 216774339- | N_Shore |
| | | | | | 216774734 | |
| cg00932063 | chr10: 126234138 | LHPP.GENEBODY | SCLC-A | R | | |
| cg02323550 | chr7: 157660321 | PTPRN2.GENEBODY | SCLC-A | F | chr7: 157659433- | Island |
| | | | | | 157660322 | |
| cg03170878 | chr7: 157630174 | PTPRN2.GENEBODY | SCLC-A | F | chr7: 157631319- | N_Shore |
| | | | | | 157631615 | |
| cg05104995 | chr10: 49460249 | FRMPD2.GENEBODY | SCLC-A | F | | |
| cg05336188 | chr19: 18959506 | UPF1.GENEBODY | SCLC-A | F | | |
| cg05987961 | chr7: 158059396 | PTPRN2.GENEBODY | SCLC-A | F | chr7: 158060894- | N_Shore |
| | | | | | 158061100 | |
| cg11376706 | chr7: 157514626 | PTPRN2.GENEBODY | SCLC-A | F | | |
| cg15752885 | chr7: 158255389 | PTPRN2.GENEBODY | SCLC-A | F | | |
| cg19487718 | chr4: 185747708 | ACSL1.TSS1500 | SCLC-A | R | chr4: 185746557- | Island |
| | | | | | 185747827 | |
| cg27282206 | chr7: 158179313 | PTPRN2.GENEBODY | SCLC-A | R | | |
| cg00108938 | chr7: 157633252 | PTPRN2.GENEBODY | SCLC-A | F | chr7: 157631319- | S_Shore |
| | | | | | 157631615 | |
| cg11111139 | chr7: 157620685 | PTPRN2.GENEBODY | SCLC-A | R | chr7: 157618856- | S_Shore |
| | | | | | 157619302 | |
| cg03347095 | chr7: 158157967 | PTPRN2.GENEBODY | SCLC-A | R | | |
| cg04772948 | chr20: 22401407 | LOC284788.TSS200 | SCLC-A | R | | |
| cg07011711 | chr11: 61467286 | DAGLA.UTR5 | SCLC-A | R | | |
| cg07037223 | chr7: 157618857 | PTPRN2.GENEBODY | SCLC-A | R | chr7: 157618856- | Island |
| | | | | | 157619302 | |
| cg10798171 | chr7: 8302301 | ICA1.TSS200 | SCLC-A | F | chr7: 8301031-8302252 | S_Shore |
| cg10817855 | chr7: 99019985 | PTCD1.GENEBODY | SCLC-A | F | chr7: 99016967- | S_Shelf |
| | | | | | 99017289 | |
| cg10932907 | chr11: 129723947 | TMEM45B.GENEBODY | SCLC-A | F | | |
| cg12030523 | chr5: 126984765 | CTXN3.EXON1 | SCLC-A | R | | |
| cg13808071 | chr2: 11679872 | GREB1.TSS1500 | SCLC-A | R | | |
| cg19285478 | chr11: 63849412 | MACROD1.GENEBODY | SCLC-A | F | chr11: 63852803- | N_Shelf |
| | | | | | 63853082 | |
| cg19564367 | chr4: 7941852 | AFAP1.TSS200 | SCLC-A | F | chr4: 7940563-7941853 | Island |
| cg24324985 | chr20: 22566884 | FOXA2.TSS1500 | SCLC-A | R | chr20: 22566821- | Island |
| | | | | | 22567055 | |
| cg26160008 | chr12: 6451035 | TNFRSF1A.UTR5 | SCLC-A | R | | |
| cg05358575 | chr2: 26764961 | OTOF.GENEBODY | SCLC-A | F | | |
| cg11855643 | chr1: 184473341 | C1orf21.GENEBODY | SCLC-A | R | | |
| cg13810707 | chr7: 122018993 | CADPS2.GENEBODY | SCLC-A | R | | |
| cg14974147 | chr3: 121310923 | FBXO40.TSS1500 | SCLC-A | F | | |
| cg00035220 | chr7: 158197980 | PTPRN2.GENEBODY | SCLC-A | F | chr7: 158198545- | N_Shore |
| | | | | | 158198907 | |
| cg04112626 | chr4: 79861272 | PAQR3.TSS1500 | SCLC-A | F | chr4: 79860213- | S_Shore |
| | | | | | 79860745 | |
| cg04873861 | chr3: 12525454 | TSEN2.TSS1500 | SCLC-A | F | chr3: 12525749- | N_Shore |
| | | | | | 12526346 | |
| cg07011231 | chr2: 73329813 | RAB11FIP5.GENEBODY | SCLC-A | F | | |
| cg15000071 | chr3: 134889493 | EPHB1.GENEBODY | SCLC-A | F | | |
| cg15090562 | chr1: 6534449 | PLEKHG5.GENEBODY | SCLC-A | F | chr1: 6530536-6531814 | S_Shelf |
| cg18187658 | chr1: 230294691 | GALNT2.GENEBODY | SCLC-A | R | | |
| cg21147709 | chr1: 12004662 | PLOD1.GENEBODY | SCLC-A | R | | |
| cg23313445 | chr11: 4792272 | OR51F1.TSS1500 | SCLC-A | F | | |
| cg23636833 | chr7: 157514822 | PTPRN2.GENEBODY | SCLC-A | F | | |
| cg26713273 | chr12: 11906243 | ETV6.GENEBODY | SCLC-A | R | | |
| cg01431993 | chr2: 182542864 | NEUROD1.GENEBODY | SCLC-A | R | | |
| cg01570265 | chr14: 70180751 | KIAA0247.UTR3 | SCLC-A | R | | |
| cg03599855 | chr8: 119073725 | EXT1.GENEBODY | SCLC-A | R | | |
| cg04303033 | chr7: 157586345 | PTPRN2.GENEBODY | SCLC-A | F | chr7: 157586711- | N_Shore |
| | | | | | 157587112 | |
| cg05241143 | chr7: 157916636 | PTPRN2.GENEBODY | SCLC-A | F | chr7: 157918704- | N_Shelf |
| | | | | | 157919409 | |
| cg05430989 | chr3: 133749309 | SLCO2A1.TSS1500 | SCLC-A | F | chr3: 133747920- | S_Shore |
| | | | | | 133748266 | |
| cg05998486 | chr7: 150645092 | KCNH2.GENEBODY | SCLC-A | R | chr7: 150644455- | S_Shore |
| | | | | | 150644767 | |
| cg14171944 | chr9: 126323477 | DENND1A.GENEBODY | SCLC-A | F | | |
| cg21443274 | chr8: 106782921 | ZFPM2.GENEBODY | SCLC-A | R | | |
| cg11717251 | chr7: 157589770 | PTPRN2.GENEBODY | SCLC-A | F | chr7: 157586711- | S_Shelf |
| | | | | | 157587112 | |
| cg25481630 | chr7: 157499673 | PTPRN2.GENEBODY | SCLC-A | R | chr7: 157503469- | N_Shelf |
| | | | | | 157504144 | |
| cg02367316 | chr7: 39125198 | POU6F2.GENEBODY | SCLC-A | F | | |
| cg03697708 | chr19: 13617549 | CACNA1A.TSS1500 | SCLC-A | F | chr19: 13616752- | S_Shore |
| | | | | | 13617267 | |
| cg04983349 | chr6: 80381447 | SH3BGRL2.GENEBODY | SCLC-A | R | | |
| cg08570034 | chr5: 175227702 | CPLX2.UTR5 | SCLC-A | F | chr5: 175223609- | S_Shelf |
| | | | | | 175224679 | |
| cg09805271 | chr11: 5010562 | MMP26.GENEBODY | SCLC-A | R | | |
| cg10531355 | chr5: 79489249 | SERINC5.GENEBODY | SCLC-A | R | | |
| cg13649658 | chr9: 140680393 | EHMT1.GENEBODY | SCLC-A | F | chr9: 140683120- | N_Shelf |
| | | | | | 140683774 | |
| cg14779376 | chr7: 157341829 | PTPRN2.GENEBODY | SCLC-A | R | | |
| cg18769909 | chr7: 157616107 | PTPRN2.GENEBODY | SCLC-A | F | chr7: 157618856- | N_Shelf |
| | | | | | 157619302 | |
| cg20808813 | chr7: 157663646 | PTPRN2.GENEBODY | SCLC-A | F | chr7: 157666069- | N_Shelf |
| | | | | | 157666399 | |
| cg26562638 | chr1: 226130366 | LEFTY2.TSS1500 | SCLC-A | F | chr1: 226127112- | S_Shelf |
| | | | | | 226127695 | |
| cg01863682 | chr2: 182545771 | NEUROD1.TSS1500 | SCLC-A | R | chr2: 182547873- | N_Shelf |
| | | | | | 182549177 | |
| cg02573468 | chr15: 41224537 | DLL4.GENEBODY | SCLC-A | F | chr15: 41217789- | S_Shore |
| | | | | | 41223180 | |
| cg02860921 | chr7: 157506297 | PTPRN2.GENEBODY | SCLC-A | F | chr7: 157503469- | S_Shelf |
| | | | | | 157504144 | |
| cg04087039 | chr7: 157697459 | PTPRN2.GENEBODY | SCLC-A | R | chr7: 157694387- | S_Shelf |
| | | | | | 157694661 | |
| cg06455686 | chr6: 168971081 | SMOC2.GENEBODY | SCLC-A | F | chr6: 168972516- | N_Shore |
| | | | | | 168974109 | |
| cg10122877 | chr4: 37585361 | C4orf19.TSS1500 | SCLC-A | F | | |
| cg10288307 | chr7: 157563602 | PTPRN2.GENEBODY | SCLC-A | R | chr7: 157560974- | S_Shelf |
| | | | | | 157561195 | |
| cg11261691 | chr20: 30914444 | KIF3B.GENEBODY | SCLC-A | R | | |
| cg13915179 | chr7: 158038007 | PTPRN2.GENEBODY | SCLC-A | R | chr7: 158037708- | Island |
| | | | | | 158038048 | |
| cg19280540 | chr19: 16553711 | EPS15L1.GENEBODY | SCLC-A | R | | |
| cg22053861 | chr11: 65318683 | LTBP3.GENEBODY | SCLC-A | R | chr11: 65321225- | N_Shelf |
| | | | | | 65321823 | |
| cg22705918 | chr2: 25518238 | DNMT3A.GENEBODY | SCLC-A | F | | |
| cg22719871 | chr11: 66105148 | BRMS1.UTR3 | SCLC-A | R | chr11: 66102024- | S_Shelf |
| | | | | | 66102279 | |
| cg24060173 | chr1: 205488243 | CDK18.UTR5 | SCLC-A | R | | |
| cg25001924 | chr7: 157919982 | PTPRN2.GENEBODY | SCLC-A | R | chr7: 157918704- | S_Shore |
| | | | | | 157919409 | |
| cg26816432 | chr7: 158165951 | PTPRN2.GENEBODY | SCLC-A | F | | |
| cg27099497 | chr7: 158269297 | PTPRN2.GENEBODY | SCLC-A | F | | |
| cg27324698 | chr7: 157916687 | PTPRN2.GENEBODY | SCLC-A | F | chr7: 157918704- | N_Shelf |
| | | | | | 157919409 | |
| cg01181415 | chr12: 16757954 | LMO3.UTR5 | SCLC-A | R | | |
| cg05552035 | chr8: 37699239 | GPR124.GENEBODY | SCLC-A | F | chr8: 37698846- | Island |
| | | | | | 37699931 | |
| cg06818217 | chr12: 97887662 | RMST.GENEBODY | SCLC-A | R | | |
| cg08141495 | chr18: 31739521 | NOL4.GENEBODY | SCLC-A | F | chr18: 31739047- | Island |
| | | | | | 31739522 | |
| cg09643186 | chr14: 65409452 | GPX2.EXON1 | SCLC-A | F | | |
| cg13432032 | chr15: 76692444 | SCAPER.GENEBODY | SCLC-A | F | | |
| cg17611686 | chr20: 22547532 | C20orf56.GENEBODY | SCLC-A | F | chr20: 22548967- | N_Shore |
| | | | | | 22549720 | |
| cg20821442 | chr7: 105279882 | ATXN7L1.GENEBODY | SCLC-A | F | | |
| cg24040043 | chr1: 207224982 | YOD1.TSS1500 | SCLC-A | F | chr1: 207226281- | N_Shore |
| | | | | | 207226842 | |
| cg24524099 | chr7: 157643007 | PTPRN2.GENEBODY | SCLC-A | R | | |
| cg02546690 | chr11: 17834480 | SERGEF.GENEBODY | SCLC-A | R | | |
| cg02932780 | chr6: 31762353 | VARS.GENEBODY | SCLC-A | R | chr6: 31763240- | N_Shore |
| | | | | | 31763905 | |
| cg04983296 | chr4: 79861284 | PAQR3.TSS1500 | SCLC-A | F | chr4: 79860213- | S_Shore |
| | | | | | 79860745 | |
| cg09367807 | chr11: 1577706 | HCCA2.GENEBODY | SCLC-A | R | chr11: 1577705-1579091 | Island |
| cg11199014 | chr19: 39798563 | LRFN1.GENEBODY | SCLC-A | R | chr19: 39797786- | Island |
| | | | | | 39799250 | |
| cg14468481 | chr7: 157561104 | PTPRN2.GENEBODY | SCLC-A | R | chr7: 157560974- | Island |
| | | | | | 157561195 | |
| cg21759685 | chr3: 46400618 | CCR2.UTR3 | SCLC-A | F | | |
| cg23629187 | chr8: 125576109 | MTSS1.GENEBODY | SCLC-A | R | | |
| cg00341732 | chr22: 31479561 | SMTN.GENEBODY | SCLC-A | F | chr22: 31480774- | N_Shore |
| | | | | | 31481373 | |
| cg00362285 | chr1: 236444212 | ERO1LB.GENEBODY | SCLC-A | F | chr1: 236444603- | N_Shore |
| | | | | | 236445700 | |
| cg01387450 | chr2: 23839710 | KLHL29.GENEBODY | SCLC-A | R | | |
| cg01557792 | chr14: 70162755 | KIAA0247.GENEBODY | SCLC-A | F | | |
| cg07926025 | chr9: 14722864 | CER1.TSS200 | SCLC-A | R | | |
| cg11939450 | chr20: 18024215 | OVOL2.GENEBODY | SCLC-A | R | | |
| cg14119936 | chr11: 4791012 | OR51F1.EXON1 | SCLC-A | F | | |
| cg15727524 | chr7: 157531216 | PTPRN2.GENEBODY | SCLC-A | F | | |
| cg16615040 | chr21: 41516861 | DSCAM.GENEBODY | SCLC-A | R | chr21: 41516237- | S_Shore |
| | | | | | 41516472 | |
| cg17491449 | chr4: 3344154 | RGS12.GENEBODY | SCLC-A | F | chr4: 3341783-3342021 | S_Shelf |
| cg17599372 | chr7: 157894874 | PTPRN2.GENEBODY | SCLC-A | F | chr7: 157890656- | S_Shelf |
| | | | | | 157891557 | |
| cg22016731 | chr1: 40311142 | TRIT1.GENEBODY | SCLC-A | F | | |
| cg22792988 | chr5: 76376904 | SNORA47.TSS1500 | SCLC-A | F | chr5: 76372882- | S_Shelf |
| | | | | | 76373720 | |
| cg23919844 | chr13: 111819481 | ARHGEF7.GENEBODY | SCLC-A | R | | |
| cg25680145 | chr6: 125623414 | HDDC2.TSS200 | SCLC-A | F | chr6: 125622704- | S_Shore |
| | | | | | 125623117 | |
| cg26480662 | chr7: 157778593 | PTPRN2.GENEBODY | SCLC-A | R | | |
| cg26674966 | chr15: 22929993 | CYFIP1.GENEBODY | SCLC-A | R | | |
| cg01782132 | chr16: 28936054 | RABEP2.GENEBODY | SCLC-A | R | chr16: 28936053- | Island |
| | | | | | 28937036 | |
| cg03788215 | chr18: 76757841 | SALL3.UTR3 | SCLC-A | F | chr18: 76751939- | S_Shelf |
| | | | | | 76755549 | |
| cg06899237 | chr6: 153353519 | RGS17.GENEBODY | SCLC-A | R | | |
| cg08278648 | chr17: 27503704 | MYO18A.UTR5 | SCLC-A | R | chr17: 27503598- | Island |
| | | | | | 27504014 | |
| cg08450080 | chr7: 157919807 | PTPRN2.GENEBODY | SCLC-A | R | chr7: 157918704- | S_Shore |
| | | | | | 157919409 | |
| cg12699647 | chr17: 80401857 | C17orf62.UTR3 | SCLC-A | R | chr17: 80401644- | Island |
| | | | | | 80401858 | |
| cg13917127 | chr9: 116932436 | COL27A1.GENEBODY | SCLC-A | F | chr9: 116930004- | S_Shelf |
| | | | | | 116930368 | |
| cg15578140 | chr7: 147718109 | MIR548F3.GENEBODY | SCLC-A | R | | |
| cg17323383 | chr3: 182896670 | MCF2L2.UTR3 | SCLC-A | F | chr3: 182896920- | N_Shore |
| | | | | | 182897381 | |
| cg18284875 | chr7: 157777033 | PTPRN2.GENEBODY | SCLC-A | F | | |
| cg20018344 | chr20: 3204138 | ITPA.UTR3 | SCLC-A | R | | |
| cg21733013 | chr7: 22359568 | RAPGEF5.UTR5 | SCLC-A | R | | |
| cg23606244 | chr11: 4704115 | OR51E2.UTR5 | SCLC-A | F | | |
| cg23808337 | chr7: 158267402 | PTPRN2.GENEBODY | SCLC-A | R | | |
| cg24424217 | chr10: 135123421 | ZNF511.GENEBODY | SCLC-A | F | chr10: 135123238- | Island |
| | | | | | 135123448 | |
| cg25455408 | chr19: 42274924 | CEACAM6.UTR3 | SCLC-A | R | | |
| cg17015844 | chr12: 103352694 | ASCL1.EXON1 | SCLC-A | F | chr12: 103351579- | Island |
| | | | | | 103352695 | |
| cg00574412 | chr17: 27892866 | ABHD15.GENEBODY | SCLC-A | F | chr17: 27893086- | N_Shore |
| | | | | | 27896078 | |
| cg05012972 | chr7: 158250129 | PTPRN2.GENEBODY | SCLC-A | R | chr7: 158250128- | Island |
| | | | | | 158250497 | |
| cg10801633 | chr1: 10579190 | PEX14.GENEBODY | SCLC-A | R | | |
| cg11623476 | chr7: 2566027 | LFNG.GENEBODY | SCLC-A | F | chr7: 2565872-2566114 | Island |
| cg12661624 | chr7: 157957646 | PTPRN2.GENEBODY | SCLC-A | F | chr7: 157956911- | S_Shore |
| | | | | | 157957273 | |
| cg13102244 | chr7: 158198569 | PTPRN2.GENEBODY | SCLC-A | R | chr7: 158198545- | Island |
| | | | | | 158198907 | |
| cg14527645 | chr9: 116977924 | COL27A1.GENEBODY | SCLC-A | F | | |
| cg14670303 | chr6: 169654035 | THBS2.UTR5 | SCLC-A | R | | |
| cg18280382 | chr11: 126225996 | ST3GAL4.TSS200 | SCLC-A | F | chr11: 126225355- | Island |
| | | | | | 126226073 | |
| cg18438823 | chr11: 4676365 | OR51E1.UTR3 | SCLC-A | R | | |
| cg19268477 | chr7: 157951471 | PTPRN2.GENEBODY | SCLC-A | F | chr7: 157951254- | Island |
| | | | | | 157951800 | |
| cg21413492 | chr6: 70990226 | COL9A1.GENEBODY | SCLC-A | F | chr6: 70992040- | N_Shore |
| | | | | | 70992912 | |
| cg22868951 | chr4: 129767588 | PHF17.GENEBODY | SCLC-A | R | | |
| cg27572053 | chr7: 157513908 | PTPRN2.GENEBODY | SCLC-A | F | | |
| cg00161124 | chr1: 234366259 | SLC35F3.GENEBODY | SCLC-A | R | chr1: 234367135- | N_Shore |
| | | | | | 234367496 | |
| cg00973286 | chr12: 6451130 | TNFRSF1A.UTR5 | SCLC-A | F | | |
| cg02544257 | chr16: 70510663 | FUK.GENEBODY | SCLC-A | R | chr16: 70507026- | S_Shelf |
| | | | | | 70507246 | |
| cg03561416 | chr6: 157254584 | ARID1B.GENEBODY | SCLC-A | F | | |
| cg07773582 | chr16: 4845285 | LOC440335.TSS200 | SCLC-A | R | | |
| cg08335767 | chr6: 34206495 | HMGA1.UTR5 | SCLC-A | F | chr6: 34202567- | S_Shore |
| | | | | | 34206193 | |
| cg08514594 | chr7: 157874020 | PTPRN2.GENEBODY | SCLC-A | F | | |
| cg09046979 | chr16: 28333134 | SBK1.UTR3 | SCLC-A | R | chr16: 28331225- | S_Shore |
| | | | | | 28332489 | |
| cg10055471 | chr1: 27240319 | NROB2.EXON1 | SCLC-A | R | | |
| cg11809826 | chr6: 168918901 | SMOC2.GENEBODY | SCLC-A | F | chr6: 168918596- | Island |
| | | | | | 168918902 | |
| cg12286857 | chr7: 157530016 | PTPRN2.GENEBODY | SCLC-A | F | chr7: 157524157- | S_Shelf |
| | | | | | 157526741 | |
| cg14495729 | chr7: 158283266 | PTPRN2.GENEBODY | SCLC-A | R | chr7: 158282214- | Island |
| | | | | | 158283382 | |
| cg15408722 | chr7: 157866922 | PTPRN2.GENEBODY | SCLC-A | F | | |
| cg16025118 | chr12: 114273678 | RBM19.GENEBODY | SCLC-A | F | | |
| cg18789624 | chr7: 30516742 | NOD1.UTR5 | SCLC-A | F | chr7: 30517987- | N_Shore |
| | | | | | 30518610 | |
| cg20673767 | chr7: 158061805 | PTPRN2.GENEBODY | SCLC-A | R | chr7: 158060894- | S_Shore |
| | | | | | 158061100 | |
| cg20979979 | chr4: 141218072 | SCOC.UTR5 | SCLC-A | R | | |
| cg22004830 | chr7: 157595428 | PTPRN2.GENEBODY | SCLC-A | F | chr7: 157593887- | S_Shore |
| | | | | | 157594128 | |
| cg24438178 | chr11: 64120354 | CCDC88B.GENEBODY | SCLC-A | F | | |
| cg25023666 | chr21: 41585021 | DSCAM.GENEBODY | SCLC-A | F | | |
| cg00235442 | chr7: 157408294 | PTPRN2.GENEBODY | SCLC-A | F | chr7: 157409846- | N_Shore |
| | | | | | 157410241 | |
| cg04435975 | chr17: 46683047 | LOC404266.GENEBODY | SCLC-A | F | chr17: 46685244- | N_Shelf |
| | | | | | 46685449 | |
| cg08462055 | chr15: 64944023 | ZNF609.GENEBODY | SCLC-A | R | | |
| cg09035925 | chr20: 19915769 | RIN2.GENEBODY | SCLC-A | R | | |
| cg13889415 | chr9: 116943379 | COL27A1.GENEBODY | SCLC-A | R | | |
| cg14799696 | chr7: 157571491 | PTPRN2.GENEBODY | SCLC-A | R | chr7: 157568163- | S_Shelf |
| | | | | | 157568404 | |
| cg18522715 | chr7: 158298958 | PTPRN2.GENEBODY | SCLC-A | F | chr7: 158301266- | N_Shelf |
| | | | | | 158301610 | |
| cg20139338 | chr11: 64120656 | CCDC88B.GENEBODY | SCLC-A | F | | |
| cg23178546 | chr7: 157710554 | PTPRN2.GENEBODY | SCLC-A | R | | |
| cg25613641 | chr4: 140587143 | MGST2.EXON1 | SCLC-A | R | | |
| cg25710298 | chr2: 286830 | FAM150B.GENEBODY | SCLC-A | F | chr2: 287353-290099 | N_Shore |
| cg01103601 | chr2: 239288872 | TRAF3IP1.GENEBODY | SCLC-A | R | | |
| cg01371493 | chr6: 168916145 | SMOC2.GENEBODY | SCLC-A | F | chr6: 168918596- | N_Shelf |
| | | | | | 168918902 | |
| cg01384111 | chr2: 11679605 | GREB1.TSS1500 | SCLC-A | F | | |
| cg02651776 | chr10: 27447805 | MASTL.GENEBODY | SCLC-A | F | chr10: 27444133- | S_Shelf |
| | | | | | 27444494 | |
| cg03021296 | chr7: 158060614 | PTPRN2.GENEBODY | SCLC-A | F | chr7: 158060894- | N_Shore |
| | | | | | 158061100 | |
| cg04750536 | chr20: 19915857 | RIN2.GENEBODY | SCLC-A | R | | |
| cg07399355 | chr1: 155163013 | MUC1.TSS1500 | SCLC-A | R | chr1: 155163434- | N_Shore |
| | | | | | 155163818 | |
| cg08271443 | chr16: 4845348 | LOC440335.TSS200 | SCLC-A | R | | |
| cg08515646 | chr7: 157394337 | PTPRN2.GENEBODY | SCLC-A | F | | |
| cg11206084 | chr7: 157780161 | PTPRN2.GENEBODY | SCLC-A | F | | |
| cg13178597 | chr6: 153405859 | RGS17.UTR5 | SCLC-A | F | | |
| cg14603031 | chr7: 2563184 | LFNG.GENEBODY | SCLC-A | R | chr7: 2563610-2564056 | N_Shore |
| cg16848873 | chr17: 46682308 | LOC404266.GENEBODY | SCLC-A | F | chr17: 46685244- | N_Shelf |
| | | | | | 46685449 | |
| cg18780243 | chr13: 32487268 | EEF1DP3.GENEBODY | SCLC-A | R | | |
| cg20146030 | chr2: 166650965 | GALNT3.TSS200 | SCLC-A | F | chr2: 166649909- | Island |
| | | | | | 166650966 | |
| cg25430878 | chr10: 1257999 | ADARB2.GENEBODY | SCLC-A | R | | |
| cg26808921 | chr6: 134373925 | SLC2A12.TSS200 | SCLC-A | F | | |
| cg02660277 | chr7: 157438616 | PTPRN2.GENEBODY | SCLC-A | F | chr7: 157440024- | N_Shore |
| | | | | | 157442721 | |
| cg03204605 | chr4: 7941837 | AFAP1.TSS200 | SCLC-A | F | chr4: 7940563-7941853 | Island |
| cg03637218 | chr5: 115209107 | AP3S1.GENEBODY | SCLC-A | F | | |
| cg06233593 | chr16: 337645 | AXIN1.UTR3 | SCLC-A | F | chr16: 335829-336034 | S_Shore |
| cg07267845 | chr7: 157345566 | PTPRN2.GENEBODY | SCLC-A | R | chr7: 157346613- | N_Shore |
| | | | | | 157347069 | |
| cg07323947 | chr10: 103896068 | PPRC1.GENEBODY | SCLC-A | R | chr10: 103892659- | S_Shelf |
| | | | | | 103893590 | |
| cg08482167 | chr6: 144066847 | PHACTR2.GENEBODY | SCLC-A | R | | |
| cg08930672 | chr7: 157932168 | PTPRN2.GENEBODY | SCLC-A | R | | |
| cg09984136 | chr6: 37190074 | TMEM217.UTR5 | SCLC-A | F | | |
| cg13847963 | chr1: 24514220 | IL28RA.TSS1500 | SCLC-A | F | chr1: 24513540- | Island |
| | | | | | 24514477 | |
| cg14653284 | chr2: 11679879 | GREB1.UTR5 | SCLC-A | R | | |
| cg14800014 | chr5: 125800764 | GRAMD3.GENEBODY | SCLC-A | R | | |
| cg16872357 | chr7: 157529444 | PTPRN2.GENEBODY | SCLC-A | R | chr7: 157524157- | S_Shelf |
| | | | | | 157526741 | |
| cg17086205 | chr7: 157543597 | PTPRN2.GENEBODY | SCLC-A | R | | |
| cg17284536 | chr6: 166891902 | RPS6KA2.GENEBODY | SCLC-A | R | | |
| cg18174678 | chr1: 42801761 | FOXJ3.TSS1500 | SCLC-A | F | chr1: 42800265- | S_Shore |
| | | | | | 42801370 | |
| cg19476622 | chr18: 20714485 | CABLES1.TSS1500 | SCLC-A | R | chr18: 20714968- | N_Shore |
| | | | | | 20717071 | |
| cg19871235 | chr12: 71552569 | TSPAN8.TSS1500 | SCLC-A | F | | |
| cg23483563 | chr2: 163625290 | KCNH7.GENEBODY | SCLC-A | F | | |
| cg00332882 | chr20: 22401271 | LOC284788.GENEBODY | SCLC-A | F | | |
| cg01076708 | chr11: 129689897 | TMEM45B.UTR5 | SCLC-A | F | chr11: 129685737- | S_Shelf |
| | | | | | 129686211 | |
| cg02708758 | chr15: 74545009 | CCDC33.GENEBODY | SCLC-A | R | | |
| cg02978297 | chr1: 94057587 | BCAR3.GENEBODY | SCLC-A | R | | |
| cg06579626 | chr2: 73495921 | FBXO41.GENEBODY | SCLC-A | R | chr2: 73495920- | Island |
| | | | | | 73496910 | |
| cg06998965 | chr5: 68710808 | MARVELD2.TSS200 | SCLC-A | R | chr5: 68710807- | Island |
| | | | | | 68711520 | |
| cg07520608 | chr2: 23627401 | KLHL29.UTR5 | SCLC-A | R | | |
| cg07696757 | chr7: 44229001 | GCK.EXON1 | SCLC-A | R | | |
| cg08826152 | chr17: 15869607 | ADORA2B.GENEBODY | SCLC-A | R | | |
| cg09967440 | chr10: 127444969 | C10orf137.GENEBODY | SCLC-A | F | | |
| cg15016771 | chr2: 235403218 | ARL4C.UTR3 | SCLC-A | R | chr2: 235404502- | N_Shore |
| | | | | | 235406541 | |
| cg15553397 | chr1: 76695186 | ST6GALNAC3.GENEBODY | SCLC-A | R | | |
| cg16666708 | chr19: 17797733 | UNC13A.GENEBODY | SCLC-A | F | chr19: 17797530- | Island |
| | | | | | 17797748 | |
| cg18351607 | chr10: 124739543 | PSTK.TSS200 | SCLC-A | R | chr10: 124739792- | N_Shore |
| | | | | | 124740292 | |
| cg19502457 | chr14: 65409354 | GPX2.EXON1 | SCLC-A | R | | |
| cg20464246 | chr1: 216774397 | ESRRG.GENEBODY | SCLC-A | R | chr1: 216774339- | Island |
| | | | | | 216774734 | |
| cg00119314 | chr17: 48555610 | RSAD1.TSS1500 | SCLC-A | F | chr17: 48556004- | N_Shore |
| | | | | | 48556509 | |
| cg01861131 | chr16: 4845708 | LOC440335.GENEBODY | SCLC-A | R | | |
| cg02356647 | chr7: 158102787 | PTPRN2.GENEBODY | SCLC-A | R | chr7: 158103336- | N_Shore |
| | | | | | 158103601 | |
| cg03391854 | chr7: 157961629 | PTPRN2.GENEBODY | SCLC-A | F | | |
| cg04476846 | chr5: 78791415 | HOMER1.GENEBODY | SCLC-A | R | | |
| cg05234415 | chr19: 38743744 | PPP1R14A.GENEBODY | SCLC-A | R | chr19: 38746638- | N_Shelf |
| | | | | | 38747379 | |
| cg10471944 | chr7: 157345246 | PTPRN2.GENEBODY | SCLC-A | F | chr7: 157346613- | N_Shore |
| | | | | | 157347069 | |
| cg10481560 | chr11: 1597444 | HCCA2.GENEBODY | SCLC-A | R | chr11: 1595809-1596143 | S_Shore |
| cg14249078 | chr11: 117665480 | DSCAML1.GENEBODY | SCLC-A | F | chr11: 117666634- | N_Shore |
| | | | | | 117668141 | |
| cg17114257 | chr11: 72854156 | FCHSD2.TSS1500 | SCLC-A | F | chr11: 72852176- | Island |
| | | | | | 72854160 | |
| cg18645642 | chr1: 8631756 | RERE.GENEBODY | SCLC-A | R | | |
| cg19948253 | chr11: 65318624 | LTBP3.GENEBODY | SCLC-A | R | chr11: 65321225- | N_Shelf |
| | | | | | 65321823 | |
| cg23097035 | chr7: 28396164 | CREB5.UTR5 | SCLC-A | R | | |
| cg23381267 | chr14: 88787370 | KCNK10.GENEBODY | SCLC-A | F | chr14: 88789600- | N_Shelf |
| | | | | | 88790493 | |
| cg25167574 | chr4: 79861398 | PAQR3.TSS1500 | SCLC-A | F | chr4: 79860213- | S_Shore |
| | | | | | 79860745 | |
| cg25644224 | chr7: 158195876 | PTPRN2.GENEBODY | SCLC-A | R | chr7: 158198545- | N_Shelf |
| | | | | | 158198907 | |
| cg26527638 | chr17: 79197398 | AZI1.TSS1500 | SCLC-A | R | chr17: 79196345- | Island |
| | | | | | 79197419 | |
| cg00405069 | chr1: 52455390 | RAB3B.UTR5 | SCLC-A | R | chr1: 52455970- | N_Shore |
| | | | | | 52456453 | |
| cg03873826 | chr16: 68407311 | SMPD3.UTR5 | SCLC-A | F | chr16: 68404907- | S_Shore |
| | | | | | 68406273 | |
| cg04937416 | chr7: 157479272 | PTPRN2.GENEBODY | SCLC-A | F | chr7: 157476886- | Island |
| | | | | | 157486719 | |
| cg04977856 | chr14: 89886941 | FOXN3.UTR5 | SCLC-A | F | chr14: 89882421- | S_Shelf |
| | | | | | 89884278 | |
| cg11115622 | chr14: 65169958 | PLEKHG3.TSS1500 | SCLC-A | R | chr14: 65170876- | N_Shore |
| | | | | | 65172007 | |
| cg16177633 | chr1: 232172585 | DISC1.UTR3 | SCLC-A | R | | |
| cg16532223 | chr1: 6542083 | PLEKHG5.UTR5 | SCLC-A | F | chr1: 6545143-6545559 | N_Shelf |
| cg17429772 | chr7: 158269409 | PTPRN2.GENEBODY | SCLC-A | F | | |
| cg18943033 | chr6: 168993309 | SMOC2.GENEBODY | SCLC-A | R | | |
| cg21240812 | chr8: 143483748 | TSNARE1.UTR5 | SCLC-A | F | chr8: 143484018- | N_Shore |
| | | | | | 143485051 | |
| cg23430388 | chr3: 63429828 | SYNPR.GENEBODY | SCLC-A | F | | |
| cg25503804 | chr11: 46739832 | F2.TSS1500 | SCLC-A | R | | |
| cg02694994 | chr7: 157612661 | PTPRN2.GENEBODY | SCLC-A | R | chr7: 157612036- | S_Shore |
| | | | | | 157612316 | |
| cg05212138 | chr20: 23063119 | CD93.UTR3 | SCLC-A | R | | |
| cg07265549 | chr16: 27607018 | KIAA0556.GENEBODY | SCLC-A | F | | |
| cg09350411 | chr7: 157667935 | PTPRN2.GENEBODY | SCLC-A | R | chr7: 157669879- | N_Shore |
| | | | | | 157670095 | |
| cg13794807 | chr4: 153871752 | FHDC1.GENEBODY | SCLC-A | R | | |
| cg15374582 | chr7: 158250524 | PTPRN2.GENEBODY | SCLC-A | F | chr7: 158247888- | S_Shore |
| | | | | | 158249312 | |
| cg25014026 | chr8: 73735989 | KCNB2.GENEBODY | SCLC-A | F | | |
| cg26775087 | chr3: 15382808 | SH3BP5.EXON1 | SCLC-A | R | | |
| cg27373591 | chr18: 70208064 | CBLN2.GENEBODY | SCLC-A | F | chr18: 70208974- | N_Shore |
| | | | | | 70211790 | |
| cg01008164 | chr11: 4825232 | OR52R1.EXON1 | SCLC-A | F | | |
| cg01008405 | chr5: 139039026 | CXXC5.UTR5 | SCLC-A | F | chr5: 139040819- | N_Shore |
| | | | | | 139041028 | |
| cg01338378 | chr7: 102036355 | PRKRIP1.TSS1500 | SCLC-A | F | chr7: 102036470- | N_Shore |
| | | | | | 102037017 | |
| cg01789980 | chr5: 167743144 | WWC1.GENEBODY | SCLC-A | R | | |
| cg04682791 | chr6: 25313030 | LRRC16A.GENEBODY | SCLC-A | F | | |
| cg08082862 | chr16: 67807580 | RANBP10.GENEBODY | SCLC-A | F | | |
| cg13917863 | chr11: 4945892 | OR51G1.TSS1500 | SCLC-A | F | | |
| cg16642299 | chr12: 7071383 | MIR200C.TSS1500 | SCLC-A | F | | |
| cg21126943 | chr19: 42259395 | CEACAM6.TSS200 | SCLC-A | F | | |
| cg21316923 | chr2: 121983716 | TFCP2L1.GENEBODY | SCLC-A | F | chr2: 121983326- | Island |
| | | | | | 121983740 | |
| cg22054191 | chr19: 42637630 | POU2F2.TSS1500 | SCLC-A | F | chr19: 42637277- | Island |
| | | | | | 42637684 | |
| cg22968167 | chr11: 4945873 | OR51G1.TSS1500 | SCLC-A | F | | |
| cg23447996 | chr9: 93564603 | SYK.GENEBODY | SCLC-A | R | chr9: 93563775- | S_Shore |
| | | | | | 93564546 | |
| cg00537335 | chr7: 157989340 | PTPRN2.GENEBODY | SCLC-A | R | | |
| cg05161074 | chr15: 42289885 | PLA2G4E.GENEBODY | SCLC-A | F | | |
| cg13068215 | chr1: 240286481 | FMN2.GENEBODY | SCLC-A | R | | |
| cg16670736 | chr7: 157777149 | PTPRN2.GENEBODY | SCLC-A | F | | |
| cg21815508 | chr7: 157530036 | PTPRN2.GENEBODY | SCLC-A | R | chr7: 157524157- | S_Shelf |
| | | | | | 157526741 | |
| cg21881338 | chr12: 125566189 | AACS.GENEBODY | SCLC-A | F | chr12: 125569828- | N_Shelf |
| | | | | | 125570140 | |
| cg22123255 | chr6: 30182088 | TRIM26.TSS1500 | SCLC-A | F | chr6: 30180933- | Island |
| | | | | | 30182187 | |
| cg23087306 | chr10: 119294055 | EMX2OS.GENEBODY | SCLC-A | F | chr10: 119293917- | Island |
| | | | | | 119297675 | |
| cg00455386 | chr7: 140104086 | RAB19.UTR5 | SCLC-A | F | | |
| cg00679546 | chr17: 60729882 | MRC2.GENEBODY | SCLC-A | R | chr17: 60729645- | Island |
| | | | | | 60730269 | |
| cg01807748 | chr1: 94534645 | ABCA4.GENEBODY | SCLC-A | R | | |
| cg03758241 | chr9: 116929797 | COL27A1.GENEBODY | SCLC-A | R | chr9: 116930004- | N_Shore |
| | | | | | 116930368 | |
| cg13151425 | chr7: 2561116 | LFNG.GENEBODY | SCLC-A | R | chr7: 2558371-2559967 | S_Shore |
| cg14415844 | chr14: 91884659 | CCDC88C.TSS1500 | SCLC-A | F | chr14: 91883672- | Island |
| | | | | | 91885367 | |
| cg19513004 | chr6: 34206683 | HMGA1.UTR5 | SCLC-A | F | chr6: 34202567- | S_Shore |
| | | | | | 34206193 | |
| cg20217702 | chr3: 134094099 | AMOTL2.TSS1500 | SCLC-A | F | chr3: 134092257- | S_Shore |
| | | | | | 134093741 | |
| cg21279806 | chr6: 169654719 | THBS2.TSS1500 | SCLC-A | R | | |
| cg22017213 | chr4: 86851225 | ARHGAP24.GENEBODY | SCLC-A | F | | |
| cg22231602 | chr1: 241176676 | RGS7.GENEBODY | SCLC-A | F | | |
| cg23358740 | chr14: 102198157 | C14orf72.GENEBODY | SCLC-A | R | | |
| cg27096172 | chr7: 158350433 | PTPRN2.GENEBODY | SCLC-A | R | chr7: 158349387- | S_Shore |
| | | | | | 158350091 | |
| cg00590039 | chr6: 111888683 | TRAF3IP2.GENEBODY | SCLC-A | F | | |
| cg03653855 | chr5: 146436093 | PPP2R2B.UTR5 | SCLC-A | R | | |
| cg03775459 | chr7: 105278448 | ATXN7L1.GENEBODY | SCLC-A | R | | |
| cg07602073 | chr1: 228595680 | TRIM17.UTR3 | SCLC-A | F | chr1: 228593811- | S_Shore |
| | | | | | 228594713 | |
| cg09682426 | chr6: 168901167 | SMOC2.GENEBODY | SCLC-A | F | | |
| cg10896862 | chr1: 161275561 | MPZ.UTR3 | SCLC-A | R | | |
| cg11534680 | chr2: 11680020 | GREB1.UTR5 | SCLC-A | R | | |
| cg14666310 | chr19: 42211479 | CEACAM5.TSS1500 | SCLC-A | F | | |
| cg16748524 | chr7: 157568392 | PTPRN2.GENEBODY | SCLC-A | F | chr7: 157568163- | Island |
| | | | | | 157568404 | |
| cg22539431 | chr7: 127423253 | SND1.GENEBODY | SCLC-A | R | | |
| cg23447239 | chr7: 157697628 | PTPRN2.GENEBODY | SCLC-A | F | chr7: 157694387- | S_Shelf |
| | | | | | 157694661 | |
| cg24996491 | chr7: 157535296 | PTPRN2.GENEBODY | SCLC-A | R | | |
| cg01259220 | chr7: 914964 | C7orf20.TSS1500 | SCLC-A | F | chr7: 915753-916644 | N_Shore |
| cg01625621 | chr15: 34260433 | AVEN.GENEBODY | SCLC-A | R | | |
| cg02671204 | chr16: 84076834 | SLC38A8.TSS1500 | SCLC-A | F | | |
| cg03512414 | chr20: 19915874 | RIN2.GENEBODY | SCLC-A | R | | |
| cg04982488 | chr10: 116103698 | AFAP1L2.GENEBODY | SCLC-A | F | | |
| cg06612016 | chr7: 158113184 | PTPRN2.GENEBODY | SCLC-A | R | chr7: 158109334- | S_Shelf |
| | | | | | 158109594 | |
| cg07543823 | chr22: 20254968 | RTN4R.GENEBODY | SCLC-A | R | chr22: 20254889- | Island |
| | | | | | 20255692 | |
| cg09985802 | chr10: 126234119 | LHPP.GENEBODY | SCLC-A | R | | |
| cg12057576 | chr15: 78918529 | CHRNB4.GENEBODY | SCLC-A | R | | |
| cg12492087 | chr15: 42749885 | ZFP106.TSS200 | SCLC-A | F | | |
| cg17772793 | chr6: 168901202 | SMOC2.GENEBODY | SCLC-A | R | | |
| cg19681352 | chr1: 27282675 | C1orf172.UTR5 | SCLC-A | R | chr1: 27286065- | N_Shelf |
| | | | | | 27287101 | |
| cg23312425 | chr11: 64120623 | CCDC88B.GENEBODY | SCLC-A | F | | |
| cg25513853 | chr17: 72272298 | DNAI2.UTR5 | SCLC-A | F | chr17: 72270301- | S_Shore |
| | | | | | 72270512 | |
| cg01312997 | chr2: 128180646 | PROC.GENEBODY | SCLC-A | R | chr2: 128180294- | Island |
| | | | | | 128181381 | |
| cg02641539 | chr8: 105351190 | TM7SF4.TSS1500 | SCLC-A | R | | |
| cg04432875 | chr13: 113411561 | ATP11A.GENEBODY | SCLC-A | R | chr13: 113410004- | S_Shore |
| | | | | | 113410362 | |
| cg06626786 | chr21: 36094858 | NCRNA00160.TSS1500 | SCLC-A | R | | |
| cg06977987 | chr11: 4902464 | OR51T1.TSS1500 | SCLC-A | R | | |
| cg07926178 | chr7: 158130463 | PTPRN2.GENEBODY | SCLC-A | R | | |
| cg09001440 | chr16: 87952767 | CASA.GENEBODY | SCLC-A | F | | |
| cg10926554 | chr6: 111888540 | TRAF3IP2.GENEBODY | SCLC-A | R | | |
| cg11863717 | chr10: 73725041 | CHST3.UTR5 | SCLC-A | R | chr10: 73723711- | S_Shore |
| | | | | | 73724770 | |
| cg12109740 | chr11: 72084034 | CLPB.GENEBODY | SCLC-A | R | | |
| cg13427810 | chr9: 116990636 | COL27A1.GENEBODY | SCLC-A | F | | |
| cg23234179 | chr12: 88420834 | C12orf50.UTR5 | SCLC-A | R | | |
| cg23520574 | chr11: 4928572 | OR51A7.TSS200 | SCLC-A | R | | |
| cg24500959 | chr10: 99474813 | MARVELD1.GENEBODY | SCLC-A | R | chr10: 99473084- | S_Shore |
| | | | | | 99473291 | |
| cg21756355 | chr7: 2233135 | MAD1L1.GENEBODY | SCLC-A | F | | |
| cg00794178 | chr3: 182897283 | MCF2L2.GENEBODY | SCLC-A | F | chr3: 182896920- | Island |
| | | | | | 182897381 | |
| cg04336561 | chr7: 157548939 | PTPRN2.GENEBODY | SCLC-A | F | chr7: 157550547- | N_Shore |
| | | | | | 157551025 | |
| cg05064223 | chr7: 157611269 | PTPRN2.GENEBODY | SCLC-A | R | chr7: 157612036- | N_Shore |
| | | | | | 157612316 | |
| cg07047280 | chr21: 41516394 | DSCAM.GENEBODY | SCLC-A | F | chr21: 41516237- | Island |
| | | | | | 41516472 | |
| cg07099407 | chr6: 43027930 | KLC4.TSS1500 | SCLC-A | R | chr6: 43026939- | S_Shore |
| | | | | | 43027841 | |
| cg07576632 | chr6: 168844079 | SMOC2.GENEBODY | SCLC-A | R | chr6: 168841818- | S_Shore |
| | | | | | 168843100 | |
| cg07865091 | chr1: 43814306 | MPL.GENEBODY | SCLC-A | R | chr1: 43814305- | Island |
| | | | | | 43815277 | |
| cg11528849 | chr2: 182542901 | NEUROD1.GENEBODY | SCLC-A | R | | |
| cg12510724 | chr12: 6484607 | SCNN1A.GENEBODY | SCLC-A | F | | |
| cg13049992 | chr5: 125800778 | GRAMD3.GENEBODY | SCLC-A | F | | |
| cg17027260 | chr2: 109892050 | SH3RF3.GENEBODY | SCLC-A | R | | |
| cg19713567 | chr17: 37895429 | GRB7.UTR5 | SCLC-A | F | | |
| cg21850830 | chr8: 124553593 | FBXO32.TSS200 | SCLC-A | R | chr8: 124552539- | Island |
| | | | | | 124554017 | |
| cg22139166 | chr5: 124071107 | ZNF608.GENEBODY | SCLC-A | F | | |
| cg22211507 | chr3: 108564871 | TRAT1.GENEBODY | SCLC-A | F | | |
| cg22676000 | chr19: 42259081 | CEACAM6.TSS1500 | SCLC-A | F | | |
| cg22862420 | chr6: 153368919 | RGS17.UTR5 | SCLC-A | R | | |
| cg25098281 | chr12: 6484713 | SCNN1A.GENEBODY | SCLC-A | F | | |
| cg25735076 | chr7: 158157881 | PTPRN2.GENEBODY | SCLC-A | R | | |
| cg26265390 | chr6: 168867606 | SMOC2.GENEBODY | SCLC-A | F | | |
| cg26308909 | chr7: 2233090 | MAD1L1.GENEBODY | SCLC-A | F | | |
| cg12487162 | chr11: 12845864 | TEAD1.GENEBODY | SCLC-A | R | | |
| cg00467296 | chr11: 4928760 | OR51A7.EXON1 | SCLC-A | R | | |
| cg00751271 | chr21: 36095899 | NCRNA00160.TSS1500 | SCLC-A | R | | |
| cg02964356 | chr12: 49883750 | SPATS2.GENEBODY | SCLC-A | R | | |
| cg03521982 | chr18: 70206471 | CBLN2.GENEBODY | SCLC-A | F | chr18: 70208974- | N_Shelf |
| | | | | | 70211790 | |
| cg05004506 | chr11: 4945422 | OR51G1.EXON1 | SCLC-A | F | | |
| cg07157169 | chr5: 126984994 | CTXN3.EXON1 | SCLC-A | F | | |
| cg08867933 | chr15: 89150842 | MIR1179.TSS1500 | SCLC-A | F | chr15: 89147660- | S_Shore |
| | | | | | 89149198 | |
| cg11686009 | chr20: 2305747 | TGM3.GENEBODY | SCLC-A | R | chr20: 2308778-2309042 | N_Shelf |
| cg12812834 | chr11: 4870255 | OR51S1.EXON1 | SCLC-A | F | | |
| cg13464573 | chr2: 7172097 | RNF144A.GENEBODY | SCLC-A | F | chr2: 7172296-7172512 | N_Shore |
| cg16826777 | chr17: 43376882 | MAP3K14.UTR5 | SCLC-A | F | | |
| cg17898069 | chr1: 234445489 | SLC35F3.GENEBODY | SCLC-A | F | | |
| cg18705776 | chr10: 5565829 | CALML3.TSS1500 | SCLC-A | R | chr10: 5567122-5567828 | N_Shore |
| cg20235871 | chr7: 158047359 | PTPRN2.GENEBODY | SCLC-A | F | | |
| cg20444218 | chr11: 73024037 | ARHGEF17.GENEBODY | SCLC-A | F | chr11: 73018452- | S_Shelf |
| | | | | | 73020563 | |
| cg20786131 | chr18: 20714496 | CABLES1.TSS1500 | SCLC-A | F | chr18: 20714968- | N_Shore |
| | | | | | 20717071 | |
| cg23181133 | chr19: 42300812 | CEACAM3.GENEBODY | SCLC-A | F | | |
| cg26169081 | chr10: 12648338 | CAMK1D.GENEBODY | SCLC-A | R | | |
| cg26430059 | chr14: 89770384 | FOXN3.GENEBODY | SCLC-A | R | | |
| cg00172803 | chr16: 84076941 | SLC38A8.TSS1500 | SCLC-A | R | | |
| cg00832555 | chr3: 169529716 | LRRC34.GENEBODY | SCLC-A | F | chr3: 169529875- | N_Shore |
| | | | | | 169530435 | |
| cg05214130 | chr12: 123945990 | SNRNP35.GENEBODY | SCLC-A | F | chr12: 123942639- | S_Shelf |
| | | | | | 123943001 | |
| cg06656027 | chr12: 50016493 | PRPF40B.TSS1500 | SCLC-A | R | chr12: 50016973- | N_Shore |
| | | | | | 50017466 | |
| cg07160163 | chr9: 93563778 | SYK.TSS1500 | SCLC-A | R | chr9: 93563775- | Island |
| | | | | | 93564546 | |
| cg08692006 | chr19: 4639992 | TNFAIP8L1.TSS200 | SCLC-A | F | chr19: 4639218-4639750 | S_Shore |
| cg11028923 | chr5: 43557644 | PAIP1.TSS1500 | SCLC-A | R | chr5: 43556419- | S_Shore |
| | | | | | 43557603 | |
| cg12364943 | chr10: 134108614 | STK32C.GENEBODY | SCLC-A | F | | |
| cg12581598 | chr1: 202537837 | PPP1R12B.GENEBODY | SCLC-A | F | | |
| cg14199629 | chr9: 84202866 | TLE1.GENEBODY | SCLC-A | F | | |
| cg16063747 | chr10: 119294173 | EMX2OS.GENEBODY | SCLC-A | R | chr10: 119293917- | Island |
| | | | | | 119297675 | |
| cg20443278 | chr17: 77962098 | TBC1D16.GENEBODY | SCLC-A | R | chr17: 77965805- | N_Shelf |
| | | | | | 77966296 | |
| cg21436938 | chr9: 116061416 | RNF183.TSS200 | SCLC-A | F | | |
| cg23235971 | chr7: 157413057 | PTPRN2.GENEBODY | SCLC-A | F | chr7: 157413901- | N_Shore |
| | | | | | 157414146 | |
| cg23361275 | chr8: 8749919 | MFHAS1.EXON1 | SCLC-A | R | chr8: 8748652-8751478 | Island |
| cg24186774 | chr14: 63757917 | RHOJ.UTR3 | SCLC-A | F | | |
| cg25223634 | chr10: 104536695 | C10orf26.GENEBODY | SCLC-A | R | | |
| cg00283524 | chr22: 44639906 | KIAA1644.UTR3 | SCLC-A | F | | |
| cg01582616 | chr7: 157595153 | PTPRN2.GENEBODY | SCLC-A | R | chr7: 157593887- | S_Shore |
| | | | | | 157594128 | |
| cg04593696 | chr16: 84076900 | SLC38A8.TSS1500 | SCLC-A | F | | |
| cg05233289 | chr11: 64120728 | CCDC88B.GENEBODY | SCLC-A | R | | |
| cg06721096 | chr7: 157336864 | PTPRN2.GENEBODY | SCLC-A | F | | |
| cg08006727 | chr9: 116061614 | RNF183.TSS1500 | SCLC-A | R | | |
| cg10173620 | chr15: 89658904 | ABHD2.UTR5 | SCLC-A | R | | |
| cg12551557 | chr12: 32308125 | BICD1.GENEBODY | SCLC-A | R | | |
| cg14638988 | chr11: 62476544 | BSCL2.TSS1500 | SCLC-A | F | chr11: 62476770- | N_Shore |
| | | | | | 62477481 | |
| cg16288617 | chr8: 61190272 | CA8.GENEBODY | SCLC-A | F | chr8: 61193312- | N_Shelf |
| | | | | | 61194195 | |
| cg16347213 | chr21: 42687940 | FAM3B.TSS1500 | SCLC-A | F | chr21: 42688746- | N_Shore |
| | | | | | 42689085 | |
| cg17801546 | chr1: 167090974 | DUSP27.GENEBODY | SCLC-A | R | chr1: 167090575- | Island |
| | | | | | 167091010 | |
| cg19434087 | chr7: 157696664 | PTPRN2.GENEBODY | SCLC-A | F | chr7: 157695025- | S_Shore |
| | | | | | 157695358 | |
| cg22797514 | chr1: 44016023 | PTPRF.GENEBODY | SCLC-A | F | | |
| cg23632744 | chr11: 4674353 | OR51E1.GENEBODY | SCLC-A | R | | |
| cg24575128 | chr3: 52502445 | NISCH.GENEBODY | SCLC-A | R | | |
| cg25641451 | chr11: 4976263 | OR51A2.EXON1 | SCLC-A | F | | |
| cg00648955 | chr2: 31456217 | EHD3.TSS1500 | SCLC-A | R | chr2: 31456740- | N_Shore |
| | | | | | 31457653 | |
| cg01759986 | chr20: 22401074 | LOC284788.GENEBODY | SCLC-A | F | | |
| cg06683080 | chr15: 40646549 | PHGR1.GENEBODY | SCLC-A | R | chr15: 40650055- | N_Shelf |
| | | | | | 40651102 | |
| cg08339915 | chr7: 157394582 | PTPRN2.GENEBODY | SCLC-A | F | | |
| cg08356705 | chr11: 68148006 | LRP5.GENEBODY | SCLC-A | R | | |
| cg09833475 | chr7: 157463804 | PTPRN2.GENEBODY | SCLC-A | R | | |
| cg14583225 | chr17: 40074010 | ACLY.UTR5 | SCLC-A | R | chr17: 40074291- | N_Shore |
| | | | | | 40075380 | |
| cg15331500 | chr22: 41779351 | TEF.GENEBODY | SCLC-A | F | chr22: 41777430- | S_Shore |
| | | | | | 41778281 | |
| cg19138905 | chr11: 4902583 | OR51T1.TSS1500 | SCLC-A | R | | |
| cg20083900 | chr7: 157624841 | PTPRN2.GENEBODY | SCLC-A | F | | |
| cg20984904 | chr7: 157529335 | PTPRN2.GENEBODY | SCLC-A | R | chr7: 157524157- | S_Shelf |
| | | | | | 157526741 | |
| cg21602520 | chr18: 60985380 | BCL2.GENEBODY | SCLC-A | F | chr18: 60985503- | N_Shore |
| | | | | | 60985741 | |
| cg24939194 | chr7: 2563611 | LFNG.GENEBODY | SCLC-A | R | chr7: 2563610-2564056 | Island |
| cg26434090 | chr11: 117665395 | DSCAML1.GENEBODY | SCLC-A | R | chr11: 117666634- | N_Shore |
| | | | | | 117668141 | |
| cg00833703 | chr16: 57809842 | KIFC3.GENEBODY | SCLC-A | R | | |
| cg03916382 | chr7: 157935049 | PTPRN2.GENEBODY | SCLC-A | R | chr7: 157937614- | N_Shelf |
| | | | | | 157937841 | |
| cg04057488 | chr6: 168920227 | SMOC2.GENEBODY | SCLC-A | R | chr6: 168918596- | S_Shore |
| | | | | | 168918902 | |
| cg05310309 | chr2: 109257429 | LIMS1.UTR5 | SCLC-A | F | | |
| cg06649856 | chr7: 158355003 | PTPRN2.GENEBODY | SCLC-A | R | | |
| cg09230014 | chr7: 157876369 | PTPRN2.GENEBODY | SCLC-A | F | | |
| cg10178498 | chr3: 124103021 | KALRN.GENEBODY | SCLC-A | F | | |
| cg11374256 | chr8: 144888445 | SCRIB.GENEBODY | SCLC-A | R | chr8: 144887111- | S_Shore |
| | | | | | 144887662 | |
| cg13393785 | chr19: 8317932 | LASS4.GENEBODY | SCLC-A | R | | |
| cg18406801 | chr2: 109294820 | LIMS1.GENEBODY | SCLC-A | F | | |
| cg22297710 | chr8: 79717861 | IL7.TSS200 | SCLC-A | F | chr8: 79716681- | S_Shore |
| | | | | | 79717403 | |
| cg24248505 | chr7: 105279790 | ATXN7L1.GENEBODY | SCLC-A | R | | |
| cg24938166 | chr2: 70941682 | ADD2.UTR5 | SCLC-A | R | | |
| cg26396492 | chr20: 19915762 | RIN2.GENEBODY | SCLC-A | R | | |
| cg09279037 | chr7: 158103293 | PTPRN2.GENEBODY | SCLC-A | F | chr7: 158103336- | N_Shore |
| | | | | | 158103601 | |
| cg16284674 | chr10: 135123337 | ZNF511.GENEBODY | SCLC-A | F | chr10: 135123238- | Island |
| | | | | | 135123448 | |
| cg01089425 | chr5: 10655518 | ANKRD33B.UTR3 | SCLC-A | R | | |
| cg03315058 | chr11: 62476542 | BSCL2.TSS1500 | SCLC-A | F | chr11: 62476770- | N_Shore |
| | | | | | 62477481 | |
| cg04713531 | chr7: 158197288 | PTPRN2.GENEBODY | SCLC-A | F | chr7: 158198545- | N_Shore |
| | | | | | 158198907 | |
| cg07285983 | chr1: 174844490 | RABGAP1L.GENEBODY | SCLC-A | F | | |
| cg10188797 | chr17: 77962048 | TBC1D16.GENEBODY | SCLC-A | F | chr17: 77965805- | N_Shelf |
| | | | | | 77966296 | |
| cg10825530 | chr11: 16023708 | SOX6.GENEBODY | SCLC-A | R | | |
| cg13685679 | chr17: 79231688 | SLC38A10.GENEBODY | SCLC-A | F | chr17: 79235486- | N_Shelf |
| | | | | | 79235751 | |
| cg22209929 | chr3: 172394700 | NCEH1.UTR5 | SCLC-A | R | | |
| cg23735602 | chr3: 178276672 | KCNMB2.UTR5 | SCLC-A | R | | |
| cg24153714 | chr10: 78057958 | C10orf11.GENEBODY | SCLC-A | F | | |
| cg24576995 | chr7: 157854008 | PTPRN2.GENEBODY | SCLC-A | F | | |
| cg24813588 | chr20: 62606499 | SAMD10.UTR3 | SCLC-A | R | chr20: 62605261- | S_Shore |
| | | | | | 62605463 | |
| cg26967186 | chr7: 157919649 | PTPRN2.GENEBODY | SCLC-A | F | chr7: 157918704- | S_Shore |
| | | | | | 157919409 | |
| cg01610165 | chr5: 124071029 | ZNF608.GENEBODY | SCLC-A | R | | |
| cg01083884 | chr13: 113641411 | MCF2L.GENEBODY | SCLC-A | R | | |
| cg08102294 | chr3: 13063918 | IQSEC1.GENEBODY | SCLC-A | F | | |
| cg08648047 | chr1: 11028561 | C1orf127.GENEBODY | SCLC-A | F | | |
| cg09337391 | chr20: 45871363 | ZMYND8.GENEBODY | SCLC-A | R | chr20: 45867654- | S_Shelf |
| | | | | | 45868111 | |
| cg10761558 | chr1: 54842205 | SSBP3.GENEBODY | SCLC-A | F | | |
| cg11747695 | chr13: 111164966 | COL4A2.UTR3 | SCLC-A | F | chr13: 111164284- | S_Shore |
| | | | | | 111164547 | |
| cg12019520 | chr4: 8012176 | ABLIM2.GENEBODY | SCLC-A | F | | |
| cg15061647 | chr7: 158249209 | PTPRN2.GENEBODY | SCLC-A | F | chr7: 158247888- | Island |
| | | | | | 158249312 | |
| cg17658976 | chr7: 158111178 | PTPRN2.GENEBODY | SCLC-A | F | chr7: 158109334- | S_Shore |
| | | | | | 158109594 | |
| cg22021832 | chr19: 42944717 | CXCL17.GENEBODY | SCLC-A | R | | |
| cg24283093 | chr3: 14481550 | SLC6A6.UTR5 | SCLC-A | F | | |
| cg25291404 | chr20: 30252580 | BCL2L1.UTR3 | SCLC-A | R | | |
| cg27096807 | chr3: 111442336 | PLCXD2.UTR3 | SCLC-A | R | | |
| cg10095968 | chr21: 45847903 | TRPM2.GENEBODY | SCLC-A | R | chr21: 45844992- | S_Shelf |
| | | | | | 45845693 | |
| cg15691129 | chrX: 68725815 | FAM155B.EXON1 | SCLC-A | R | chrX: 68723731- | Island |
| | | | | | 68726051 | |
| cg23631070 | chrX: 17652804 | NHS.TSS1500 | SCLC-A | R | | |
| cg00254714 | chr20: 2309923 | TGM3.GENEBODY | SCLC-A | R | chr20: 2308778-2309042 | S_Shore |
| cg02624246 | chr12: 7073264 | MIR141.GENEBODY | SCLC-A | F | | |
| cg03079132 | chr16: 727212 | RHBDL1.GENEBODY | SCLC-A | R | chr16: 729438-735815 | N_Shelf |
| cg05195085 | chr7: 157951403 | PTPRN2.GENEBODY | SCLC-A | F | chr7: 157951254- | Island |
| | | | | | 157951800 | |
| cg05308556 | chr7: 157408770 | PTPRN2.GENEBODY | SCLC-A | F | chr7: 157409846- | N_Shore |
| | | | | | 157410241 | |
| cg06164804 | chr2: 240044389 | HDAC4.GENEBODY | SCLC-A | R | chr2: 240046200- | N_Shore |
| | | | | | 240046736 | |
| cg06675455 | chr11: 4902491 | OR51T1.TSS1500 | SCLC-A | R | | |
| cg09958065 | chr10: 12648526 | CAMK1D.GENEBODY | SCLC-A | R | | |
| cg10249637 | chr7: 157655436 | PTPRN2.GENEBODY | SCLC-A | F | chr7: 157659433- | N_Shelf |
| | | | | | 157660322 | |
| cg10999000 | chr13: 111180025 | RAB20.GENEBODY | SCLC-A | F | | |
| cg11161846 | chr1: 160990452 | F11R.GENEBODY | SCLC-A | R | chr1: 160990718- | N_Shore |
| | | | | | 160991225 | |
| cg11859489 | chr15: 92459058 | SLCO3A1.GENEBODY | SCLC-A | F | chr15: 92459266- | N_Shore |
| | | | | | 92459678 | |
| cg12817840 | chr5: 124070989 | ZNF608.GENEBODY | SCLC-A | F | | |
| cg16964025 | chr7: 157482215 | PTPRN2.GENEBODY | SCLC-A | F | chr7: 157476886- | Island |
| | | | | | 157486719 | |
| cg19404444 | chr1: 2164602 | SKI.GENEBODY | SCLC-A | R | chr1: 2158212-2161173 | S_Shelf |
| cg22531685 | chr7: 158264663 | PTPRN2.GENEBODY | SCLC-A | R | | |
| cg25003275 | chr4: 7940128 | AFAP1.UTR5 | SCLC-A | F | chr4: 7940563-7941853 | N_Shore |
| cg25541259 | chr2: 230576135 | DNER.GENEBODY | SCLC-A | R | chr2: 230578062- | N_Shore |
| | | | | | 230579981 | |
| cg00866953 | chr1: 15417078 | KIAA1026.GENEBODY | SCLC-A | F | | |
| cg01315919 | chr20: 22401404 | LOC284788.TSS200 | SCLC-A | F | | |
| cg08705382 | chr1: 230322024 | GALNT2.GENEBODY | SCLC-A | F | | |
| cg15572489 | chr7: 157481091 | PTPRN2.GENEBODY | SCLC-A | R | chr7: 157476886- | Island |
| | | | | | 157486719 | |
| cg21759331 | chr2: 209352598 | PTH2R.GENEBODY | SCLC-A | R | | |
| cg05439204 | chr7: 157854693 | PTPRN2.GENEBODY | SCLC-A | F | | |
| cg08240778 | chr11: 1643787 | HCCA2.GENEBODY | SCLC-A | F | | |
| cg08515811 | chr17: 77921489 | TBC1D16.GENEBODY | SCLC-A | R | chr17: 77922721- | N_Shore |
| | | | | | 77922973 | |
| cg08845722 | chr2: 46041801 | PRKCE.GENEBODY | SCLC-A | F | | |
| cg09314984 | chr19: 5627625 | SAFB.GENEBODY | SCLC-A | R | chr19: 5622248-5623712 | S_Shelf |
| cg09353345 | chr15: 78918560 | CHRNB4.GENEBODY | SCLC-A | R | | |
| cg10520790 | chr4: 16030846 | PROM1.GENEBODY | SCLC-A | R | | |
| cg14157855 | chr1: 32226424 | BAI2.UTR5 | SCLC-A | R | chr1: 32226146- | Island |
| | | | | | 32226535 | |
| cg16717192 | chr7: 4265800 | SDK1.GENEBODY | SCLC-A | R | | |
| cg19307500 | chr19: 1083193 | HMHA1.GENEBODY | SCLC-A | R | chr19: 1082576-1083430 | Island |
| cg21660130 | chr1: 16370243 | CLCNKB.TSS200 | SCLC-A | R | | |
| cg22531371 | chr1: 155162756 | MUC1.TSS200 | SCLC-A | F | chr1: 155163434- | N_Shore |
| | | | | | 155163818 | |
| cg02690968 | chr11: 35683923 | TRIM44.TSS1500 | SCLC-A | F | chr11: 35684421- | N_Shore |
| | | | | | 35684846 | |
| cg03337035 | chr19: 42258336 | CEACAM6.TSS1500 | SCLC-A | R | | |
| cg03694261 | chr11: 122753977 | C11orf63.UTR5 | SCLC-A | F | chr11: 122753386- | S_Shore |
| | | | | | 122753951 | |
| cg04701181 | chr11: 1606344 | HCCA2.GENEBODY | SCLC-A | F | | |
| cg05327192 | chr11: 75133593 | KLHL35.UTR3 | SCLC-A | F | chr11: 75136407- | N_Shelf |
| | | | | | 75136689 | |
| cg06294561 | chr3: 141161580 | ZBTB38.GENEBODY | SCLC-A | F | | |
| cg12149911 | chr8: 61194292 | CA8.TSS1500 | SCLC-A | R | chr8: 61193312- | S_Shore |
| | | | | | 61194195 | |
| cg14651919 | chr17: 74075189 | ZACN.TSS200 | SCLC-A | R | chr17: 74070404- | S_Shore |
| | | | | | 74073530 | |
| cg15221831 | chr19: 42408464 | ARHGEF1.GENEBODY | SCLC-A | F | chr19: 42408315- | Island |
| | | | | | 42408535 | |
| cg16537859 | chrX: 70440209 | BCYRN1.GENEBODY | SCLC-A | F | chrX: 70444046- | N_Shelf |
| | | | | | 70444270 | |
| cg22544881 | chr7: 130712346 | FLJ43663.GENEBODY | SCLC-A | R | | |
| cg24178676 | chr5: 172264981 | ERGIC1.GENEBODY | SCLC-A | F | chr5: 172261145- | S_Shelf |
| | | | | | 172261671 | |
| cg25205321 | chr13: 111096339 | COL4A2.GENEBODY | SCLC-A | R | | |
| cg00234370 | chr1: 36021674 | KIAA0319L.UTR5 | SCLC-A | F | chr1: 36022680- | N_Shore |
| | | | | | 36023834 | |
| cg00806680 | chr6: 41561085 | FOXP4.GENEBODY | SCLC-A | F | chr6: 41561221- | N_Shore |
| | | | | | 41561473 | |
| cg06716730 | chr17: 35851459 | DUSP14.UTR5 | SCLC-A | R | chr17: 35849290- | S_Shore |
| | | | | | 35850483 | |
| cg21521989 | chr7: 158269194 | PTPRN2.GENEBODY | SCLC-A | F | | |
| cg02855558 | chr7: 158107723 | PTPRN2.GENEBODY | SCLC-A | F | chr7: 158109334- | N_Shore |
| | | | | | 158109594 | |
| cg04204246 | chr6: 112537238 | LAMA4.GENEBODY | SCLC-A | R | | |
| cg05087455 | chr2: 7171869 | RNF144A.GENEBODY | SCLC-A | R | chr2: 7172296-7172512 | N_Shore |
| cg05295557 | chr14: 36988446 | NKX2-1.GENEBODY | SCLC-A | F | chr14: 36986362- | Island |
| | | | | | 36990576 | |
| cg05913555 | chr7: 158158760 | PTPRN2.GENEBODY | SCLC-A | R | | |
| cg11938455 | chr6: 15622032 | DTNBP1.GENEBODY | SCLC-A | R | | |
| cg13417891 | chr17: 35836996 | TADA2A.GENEBODY | SCLC-A | F | | |
| cg14672994 | chr17: 48503057 | ACSF2.TSS1500 | SCLC-A | R | chr17: 48503056- | Island |
| | | | | | 48503887 | |
| cg20313969 | chr2: 112939562 | FBLN7.GENEBODY | SCLC-A | F | chr2: 112939287- | Island |
| | | | | | 112939563 | |
| cg23540651 | chr1: 89490034 | GBP3.TSS1500 | SCLC-A | R | | |
| cg24611996 | chr10: 120353499 | PRLHR.UTR3 | SCLC-A | R | chr10: 120353692- | N_Shore |
| | | | | | 120355821 | |
| cg00735611 | chr17: 37895886 | GRB7.UTR5 | SCLC-A | R | | |
| cg04645676 | chr5: 10655569 | ANKRD33B.UTR3 | SCLC-A | F | | |
| cg04854917 | chr10: 131378330 | MGMT.GENEBODY | SCLC-A | R | | |
| cg09149842 | chr20: 31351091 | DNMT3B.UTR5 | SCLC-A | R | chr20: 31350149- | Island |
| | | | | | 31351263 | |
| cg13167816 | chr14: 24601808 | FITM1.GENEBODY | SCLC-A | F | chr14: 24601498- | S_Shore |
| | | | | | 24601700 | |
| cg14753321 | chr5: 176784914 | RGS14.UTR5 | SCLC-A | F | | |
| cg15736062 | chr7: 158136485 | PTPRN2.GENEBODY | SCLC-A | R | | |
| cg15978565 | chr2: 11894464 | LPIN1.UTR5 | SCLC-A | R | | |
| cg16427983 | chr12: 123453858 | ABCB9.UTR5 | SCLC-A | F | chr12: 123450868- | S_Shelf |
| | | | | | 123451207 | |
| cg16809526 | chr16: 46917681 | GPT2.TSS1500 | SCLC-A | F | chr16: 46917849- | N_Shore |
| | | | | | 46918957 | |
| cg17019292 | chr5: 68710813 | MARVELD2.TSS200 | SCLC-A | R | chr5: 68710807- | Island |
| | | | | | 68711520 | |
| cg27270684 | chr7: 55757158 | FKBP9L.TSS200 | SCLC-A | R | | |
| cg27470486 | chr17: 40073688 | ACLY.UTR5 | SCLC-A | F | chr17: 40074291- | N_Shore |
| | | | | | 40075380 | |
| cg02711212 | chr15: 69854024 | LOC145837.TSS200 | SCLC-A | F | | |
| cg04810063 | chr16: 88703634 | IL17C.TSS1500 | SCLC-A | F | chr16: 88706211- | N_Shelf |
| | | | | | 88706492 | |
| cg06313349 | chr7: 157494424 | PTPRN2.GENEBODY | SCLC-A | R | chr7: 157494510- | N_Shore |
| | | | | | 157494739 | |
| cg11577716 | chr20: 20258001 | C20orf26.GENEBODY | SCLC-A | F | | |
| cg12144100 | chr7: 157890277 | PTPRN2.GENEBODY | SCLC-A | R | chr7: 157890656- | N_Shore |
| | | | | | 157891557 | |
| cg14689490 | chr7: 158210489 | PTPRN2.GENEBODY | SCLC-A | R | | |
| cg17959824 | chr11: 70391706 | SHANK2.GENEBODY | SCLC-A | F | | |
| cg21781706 | chr1: 6438350 | ACOT7.GENEBODY | SCLC-A | F | | |
| cg24634333 | chr6: 111887834 | TRAF3IP2.GENEBODY | SCLC-A | R | | |
| cg06520307 | chr7: 149417640 | KRBA1.GENEBODY | SCLC-A | F | chr7: 149416662- | S_Shore |
| | | | | | 149416974 | |
| cg07780818 | chr3: 133749284 | SLCO2A1.TSS1500 | SCLC-A | R | chr3: 133747920- | S_Shore |
| | | | | | 133748266 | |
| cg07790176 | chr13: 92601293 | GPC5.GENEBODY | SCLC-A | R | | |
| cg09635395 | chr4: 146601470 | C4orf51.EXON1 | SCLC-A | F | | |
| cg14497491 | chr17: 77982477 | TBC1D16.GENEBODY | SCLC-A | F | chr17: 77983980- | N_Shore |
| | | | | | 77984482 | |
| cg15318176 | chr7: 157345827 | PTPRN2.GENEBODY | SCLC-A | F | chr7: 157346613- | N_Shore |
| | | | | | 157347069 | |
| cg16792062 | chr11: 128458153 | ETS1.TSS1500 | SCLC-A | R | | |
| cg17602451 | chr18: 60985645 | BCL2.GENEBODY | SCLC-A | R | chr18: 60985503- | Island |
| | | | | | 60985741 | |
| cg19628299 | chr6: 168840641 | SMOC2.TSS1500 | SCLC-A | F | chr6: 168841438- | N_Shore |
| | | | | | 168841699 | |
| cg20731121 | chr3: 111636305 | PHLDB2.GENEBODY | SCLC-A | F | | |
| cg25871713 | chr3: 37823949 | ITGA9.GENEBODY | SCLC-A | R | | |
| cg26924822 | chr4: 26332763 | RBPJ.GENEBODY | SCLC-A | F | | |
| cg00542975 | chr2: 122392291 | CLASP1.UTR5 | SCLC-A | R | | |
| cg01961752 | chr1: 1109012 | TTLL10.TSS1500 | SCLC-A | R | chr1: 1109314-1110145 | N_Shore |
| cg02369113 | chr7: 157637560 | PTPRN2.GENEBODY | SCLC-A | R | | |
| cg03486974 | chr16: 2199702 | RAB26.GENEBODY | SCLC-A | R | chr16: 2198455-2199129 | S_Shore |
| cg03767531 | chr2: 182545572 | NEUROD1.TSS200 | SCLC-A | F | chr2: 182547873- | N_Shelf |
| | | | | | 182549177 | |
| cg04143211 | chr14: 91087927 | TTC7B.GENEBODY | SCLC-A | R | chr14: 91087475- | S_Shore |
| | | | | | 91087796 | |
| cg04489012 | chr1: 151812710 | LOC100132111.GENEBODY | SCLC-A | F | chr1: 151810460- | S_Shore |
| | | | | | 151811684 | |
| cg05942111 | chr12: 65044027 | RASSF3.GENEBODY | SCLC-A | R | | |
| cg06497408 | chr7: 158046838 | PTPRN2.GENEBODY | SCLC-A | F | | |
| cg07243405 | chr7: 915203 | C7orf20.TSS1500 | SCLC-A | R | chr7: 915753-916644 | N_Shore |
| cg15490801 | chr14: 30054523 | PRKD1.GENEBODY | SCLC-A | F | | |
| cg17316096 | chr7: 158158218 | PTPRN2.GENEBODY | SCLC-A | R | | |
| cg18470891 | chr12: 2986957 | FOXM1.TSS1500 | SCLC-A | R | chr12: 2985978-2986639 | S_Shore |
| cg21790851 | chr13: 73634643 | KLF5.GENEBODY | SCLC-A | F | chr13: 73632860- | S_Shore |
| | | | | | 73634370 | |
| cg24580076 | chr7: 915073 | C7orf20.TSS1500 | SCLC-A | F | chr7: 915753-916644 | N_Shore |
| cg25727520 | chr2: 37576821 | QPCT.GENEBODY | SCLC-A | R | | |
| cg26003056 | chr5: 89908195 | GPR98.GENEBODY | SCLC-A | F | | |
| cg26231267 | chr10: 12648032 | CAMK1D.GENEBODY | SCLC-A | R | | |
| cg00072720 | chr17: 7165831 | CLDN7.TSS200 | SCLC-A | R | chr17: 7164285-7166245 | Island |
| cg02364642 | chr12: 58005758 | GEFT.EXON1 | SCLC-A | R | chr12: 58003880- | S_Shore |
| | | | | | 58004249 | |
| cg02643580 | chr12: 95671146 | VEZT.GENEBODY | SCLC-A | R | | |
| cg02836529 | chr2: 182544413 | NEUROD1.UTR5 | SCLC-A | R | chr2: 182547873- | N_Shelf |
| | | | | | 182549177 | |
| cg03641492 | chr6: 168859954 | SMOC2.GENEBODY | SCLC-A | R | | |
| cg04048740 | chr2: 236442999 | AGAP1.GENEBODY | SCLC-A | F | chr2: 236443815- | N_Shore |
| | | | | | 236444025 | |
| cg04162999 | chr11: 64120313 | CCDC88B.GENEBODY | SCLC-A | R | | |
| cg06400336 | chr8: 103136701 | NCALD.TSS200 | SCLC-A | R | chr8: 103135914- | Island |
| | | | | | 103136775 | |
| cg10198932 | chr21: 43136287 | C21orf129.TSS1500 | SCLC-A | F | | |
| cg10308629 | chr7: 134354803 | BPGM.GENEBODY | SCLC-A | R | | |
| cg12813108 | chr7: 99719912 | CNPY4.GENEBODY | SCLC-A | R | | |
| cg15906675 | chr4: 90171420 | GPRIN3.UTR5 | SCLC-A | R | | |
| cg17093877 | chr17: 72206412 | MGC16275.GENEBODY | SCLC-A | F | chr17: 72208474- | N_Shelf |
| | | | | | 72208902 | |
| cg18214661 | chr8: 17471997 | PDGFRL.GENEBODY | SCLC-A | R | | |
| cg00279797 | chrX: 109561918 | AMMECR1.UTR5 | SCLC-A | R | chrX: 109560703- | S_Shore |
| | | | | | 109561534 | |
| cg01138652 | chr22: 19973978 | ARVCF.GENEBODY | SCLC-A | F | chr22: 19974274- | N_Shore |
| | | | | | 19974756 | |
| cg04457354 | chr6: 20447442 | E2F3.GENEBODY | SCLC-A | F | | |
| cg05564438 | chr11: 64120286 | CCDC88B.GENEBODY | SCLC-A | R | | |
| cg07120097 | chr14: 37257605 | SLC25A21.GENEBODY | SCLC-A | R | | |
| cg14362920 | chr7: 157332376 | PTPRN2.UTR3 | SCLC-A | R | | |
| cg21763058 | chr12: 49740976 | DNAJC22.TSS200 | SCLC-A | R | chr12: 49738680- | S_Shore |
| | | | | | 49740841 | |
| cg22193393 | chr15: 42290845 | PLA2G4E.GENEBODY | SCLC-A | R | | |
| cg23392097 | chr11: 4704749 | OR51E2.UTR5 | SCLC-A | R | | |
| cg26379258 | chr6: 111888446 | TRAF3IP2.GENEBODY | SCLC-A | R | | |
| cg02753354 | chr19: 1074727 | HMHA1.GENEBODY | SCLC-A | R | chr19: 1074726-1075071 | Island |
| cg04531425 | chr2: 182322841 | ITGA4.GENEBODY | SCLC-A | F | chr2: 182321761- | Island |
| | | | | | 182323029 | |
| cg04955511 | chr7: 14801527 | DGKB.GENEBODY | SCLC-A | F | | |
| cg05093113 | chr20: 49200925 | MIR645.TSS1500 | SCLC-A | F | | |
| cg06979020 | chr16: 69660388 | NFAT5.UTR5 | SCLC-A | R | | |
| cg07756135 | chr4: 86748944 | ARHGAP24.GENEBODY | SCLC-A | F | | |
| cg09832443 | chr7: 28612680 | CREB5.GENEBODY | SCLC-A | F | | |
| cg13464117 | chr7: 157348161 | PTPRN2.GENEBODY | SCLC-A | R | chr7: 157346613- | S_Shore |
| | | | | | 157347069 | |
| cg14141549 | chr7: 157919408 | PTPRN2.GENEBODY | SCLC-A | F | chr7: 157918704- | Island |
| | | | | | 157919409 | |
| cg15353612 | chr4: 52904965 | SGCB.TSS1500 | SCLC-A | F | chr4: 52904454- | S_Shore |
| | | | | | 52904912 | |
| cg20978600 | chr3: 40566157 | ZNF621.TSS1500 | SCLC-A | R | chr3: 40566129- | Island |
| | | | | | 40566689 | |
| cg21191737 | chr9: 15306337 | TTC39B.GENEBODY | SCLC-A | F | chr9: 15306722- | N_Shore |
| | | | | | 15307386 | |
| cg23574281 | chr2: 47716 | FAM110C.TSS1500 | SCLC-A | R | chr2: 45510-46559 | S_Shore |
| cg00459232 | chr12: 6309025 | CD9.TSS1500 | SCLC-A | R | chr12: 6309152-6310392 | N_Shore |
| cg02479744 | chr6: 16303089 | ATXN1.UTR3 | SCLC-A | R | | |
| cg02735652 | chr1: 32226179 | BAI2.UTR5 | SCLC-A | R | chr1: 32226146- | Island |
| | | | | | 32226535 | |
| cg05175762 | chr7: 157987088 | PTPRN2.GENEBODY | SCLC-A | F | | |
| cg05722741 | chr6: 168897588 | SMOC2.GENEBODY | SCLC-A | F | | |
| cg08134671 | chr19: 2542837 | GNG7.UTR5 | SCLC-A | R | chr19: 2543638-2543878 | N_Shore |
| cg09614629 | chr7: 157985057 | PTPRN2.GENEBODY | SCLC-A | R | chr7: 157980786- | S_Shelf |
| | | | | | 157981462 | |
| cg10536786 | chr1: 7550036 | CAMTA1.GENEBODY | SCLC-A | R | | |
| cg10754395 | chr7: 158109694 | PTPRN2.GENEBODY | SCLC-A | F | chr7: 158110569- | N_Shore |
| | | | | | 158110881 | |
| cg11980090 | chr6: 30181771 | TRIM26.TSS1500 | SCLC-A | R | chr6: 30180933- | Island |
| | | | | | 30182187 | |
| cg12419766 | chr7: 157620445 | PTPRN2.GENEBODY | SCLC-A | R | chr7: 157618856- | S_Shore |
| | | | | | 157619302 | |
| cg12882572 | chr17: 35851204 | DUSP14.UTR5 | SCLC-A | R | chr17: 35849290- | S_Shore |
| | | | | | 35850483 | |
| cg18822036 | chr8: 131371659 | ASAP1.GENEBODY | SCLC-A | R | chr8: 131370087- | S_Shore |
| | | | | | 131371317 | |
| cg20535931 | chr7: 97615658 | OCM2.GENEBODY | SCLC-A | F | | |
| cg22356339 | chr12: 103352454 | ASCL1.EXON1 | SCLC-A | R | chr12: 103351579- | Island |
| | | | | | 103352695 | |
| cg26085804 | chr11: 72854242 | FCHSD2.TSS1500 | SCLC-A | F | chr11: 72852176- | S_Shore |
| | | | | | 72854160 | |
| cg27567922 | chr3: 195480636 | MUC4.GENEBODY | SCLC-A | F | chr3: 195477711- | S_Shelf |
| | | | | | 195477983 | |
| cg00816406 | chr1: 240286626 | FMN2.GENEBODY | SCLC-A | F | | |
| cg01718785 | chr7: 157916279 | PTPRN2.GENEBODY | SCLC-A | F | chr7: 157918704- | N_Shelf |
| | | | | | 157919409 | |
| cg02601893 | chr22: 43523016 | BIK.GENEBODY | SCLC-A | F | | |
| cg13374528 | chr7: 129414627 | MIR96.TSS200 | SCLC-A | R | chr7: 129418286- | N_Shelf |
| | | | | | 129422509 | |
| cg13689591 | chr3: 124239629 | KALRN.GENEBODY | SCLC-A | F | | |
| cg14575854 | chr17: 37896175 | GRB7.UTR5 | SCLC-A | F | | |
| cg20208009 | chr22: 19974048 | ARVCF.GENEBODY | SCLC-A | F | chr22: 19974274- | N_Shore |
| | | | | | 19974756 | |
| cg26572392 | chr10: 24496943 | KIAA1217.UTR5 | SCLC-A | R | | |
| cg26572992 | chr19: 18876246 | CRTC1.GENEBODY | SCLC-A | F | chr19: 18873019- | S_Shelf |
| | | | | | 18873349 | |
| cg26593067 | chr7: 157446092 | PTPRN2.GENEBODY | SCLC-A | R | chr7: 157448240- | N_Shelf |
| | | | | | 157448566 | |
| cg00325866 | chr15: 62359130 | C2CD4A.TSS200 | SCLC-A | F | chr15: 62359827- | N_Shore |
| | | | | | 62360995 | |
| cg00690082 | chr17: 40438511 | STAT5A.TSS1500 | SCLC-A | R | chr17: 40440188- | N_Shore |
| | | | | | 40441014 | |
| cg00929655 | chr15: 92459909 | SLCO3A1.GENEBODY | SCLC-A | R | chr15: 92459266- | S_Shore |
| | | | | | 92459678 | |
| cg01760027 | chrX: 49832257 | CLCN5.GENEBODY | SCLC-A | R | | |
| cg01983725 | chr1: 227161799 | CABC1.GENEBODY | SCLC-A | F | chr1: 227163739- | N_Shore |
| | | | | | 227164237 | |
| cg04868764 | chr15: 41137019 | SPINT1.GENEBODY | SCLC-A | F | chr15: 41135719- | Island |
| | | | | | 41137210 | |
| cg13700912 | chr7: 1968348 | MAD1L1.GENEBODY | SCLC-A | R | chr7: 1967664-1967994 | S_Shore |
| cg14817241 | chr19: 4851575 | PLIN3.GENEBODY | SCLC-A | F | chr19: 4851962-4852248 | N_Shore |
| cg16872071 | chr9: 135997155 | RALGDS.GENEBODY | SCLC-A | F | chr9: 135995969- | S_Shore |
| | | | | | 135996954 | |
| cg20867746 | chr11: 4871813 | OR51S1.TSS1500 | SCLC-A | R | | |
| cg21376148 | chr17: 10276260 | MYH13.UTR5 | SCLC-A | R | | |
| cg01352090 | chr16: 4103533 | ADCY9.GENEBODY | SCLC-A | F | | |
| cg01808739 | chr1: 93912802 | FNBP1L.TSS1500 | SCLC-A | F | chr1: 93913636- | N_Shore |
| | | | | | 93914524 | |
| cg02058628 | chr1: 2164542 | SKI.GENEBODY | SCLC-A | F | chr1: 2158212-2161173 | S_Shelf |
| cg07219844 | chr10: 135123764 | ZNF511.GENEBODY | SCLC-A | F | chr10: 135121991- | S_Shore |
| | | | | | 135122669 | |
| cg09526758 | chr12: 16758047 | LMO3.UTR5 | SCLC-A | F | | |
| cg14507792 | chr9: 95574913 | ANKRD19.GENEBODY | SCLC-A | R | chr9: 95569429- | S_Shelf |
| | | | | | 95572255 | |
| cg16609181 | chr19: 1083007 | HMHA1.GENEBODY | SCLC-A | R | chr19: 1082576-1083430 | Island |
| cg22337375 | chr7: 157351042 | PTPRN2.GENEBODY | SCLC-A | R | chr7: 157352995- | N_Shore |
| | | | | | 157353232 | |
| cg00926318 | chr20: 25177340 | ENTPD6.UTR5 | SCLC-A | R | chr20: 25176027- | S_Shore |
| | | | | | 25176620 | |
| cg02051806 | chr12: 86650107 | MGAT4C.UTR5 | SCLC-A | R | | |
| cg02051924 | chr16: 11681482 | LITAF.TSS200 | SCLC-A | R | chr16: 11680008- | S_Shore |
| | | | | | 11681275 | |
| cg05767421 | chr8: 120221185 | MAL2.GENEBODY | SCLC-A | R | chr8: 120220388- | Island |
| | | | | | 120221222 | |
| cg06779248 | chr7: 95458266 | DYNC111.GENEBODY | SCLC-A | R | | |
| cg10709461 | chr20: 22402729 | LOC284788.TSS1500 | SCLC-A | F | | |
| cg12379775 | chr20: 62669791 | NCRNA00176.GENEBODY | SCLC-A | F | chr20: 62669304- | Island |
| | | | | | 62670730 | |
| cg13265740 | chr6: 139349120 | C6orf115.TSS1500 | SCLC-A | R | chr6: 139349955- | N_Shore |
| | | | | | 139350740 | |
| cg14192174 | chr2: 29204117 | FAM179A.TSS200 | SCLC-A | F | | |
| cg18856478 | chr1: 43814358 | MPL.GENEBODY | SCLC-A | R | chr1: 43814305- | Island |
| | | | | | 43815277 | |
| cg20697464 | chr2: 218793716 | TNS1.UTR5 | SCLC-A | R | | |
| cg23637314 | chr1: 216774226 | ESRRG.GENEBODY | SCLC-A | F | chr1: 216774339- | N_Shore |
| | | | | | 216774734 | |
| cg24728949 | chr5: 106820169 | EFNA5.GENEBODY | SCLC-A | F | | |
| cg26215113 | chr2: 234369962 | DGKD.GENEBODY | SCLC-A | R | chr2: 234369786- | Island |
| | | | | | 234370099 | |
| cg00008819 | chr4: 3257791 | C4orf44.UTR3 | SCLC-A | F | chr4: 3254957-3255175 | S_Shelf |
| cg00115178 | chr10: 12648176 | CAMK1D.GENEBODY | SCLC-A | R | | |
| cg02276086 | chr2: 163175335 | IFIH1.TSS1500 | SCLC-A | F | | |
| cg02707152 | chr11: 64980819 | SLC22A20.TSS1500 | SCLC-A | R | | |
| cg12538248 | chr8: 61194072 | CA8.TSS200 | SCLC-A | R | chr8: 61193312- | Island |
| | | | | | 61194195 | |
| cg12806381 | chr1: 27114177 | PIGV.TSS1500 | SCLC-A | F | chr1: 27114118- | Island |
| | | | | | 27114692 | |
| cg21292473 | chr1: 181683239 | CACNA1E.GENEBODY | SCLC-A | R | | |
| cg26392737 | chr8: 21916853 | EPB49.EXON1 | SCLC-A | F | chr8: 21914275- | S_Shelf |
| | | | | | 21914525 | |
| cg00797278 | chr3: 32442928 | CMTM7.GENEBODY | SCLC-A | F | chr3: 32443152- | N_Shore |
| | | | | | 32443455 | |
| cg01435515 | chr1: 151255742 | ZNF687.UTR5 | SCLC-A | R | chr1: 151253658- | Island |
| | | | | | 151255769 | |
| cg02710090 | chr1: 207225128 | PFKFB2.TSS1500 | SCLC-A | F | chr1: 207226281- | N_Shore |
| | | | | | 207226842 | |
| cg04633683 | chr16: 29837056 | MVP.UTR5 | SCLC-A | R | | |
| cg04905719 | chr6: 134617250 | SGK1.GENEBODY | SCLC-A | F | | |
| cg09035736 | chr22: 38599166 | MAFF.UTR5 | SCLC-A | F | chr22: 38597785- | S_Shore |
| | | | | | 38598988 | |
| cg09556527 | chr1: 1239259 | ACAP3.GENEBODY | SCLC-A | F | chr1: 1242400-1245185 | N_Shelf |
| cg11315900 | chr7: 157605970 | PTPRN2.GENEBODY | SCLC-A | R | | |
| cg22581896 | chr7: 4779225 | FOXK1.GENEBODY | SCLC-A | R | | |
| cg25124966 | chr11: 129723987 | TMEM45B.GENEBODY | SCLC-A | R | | |
| cg25718402 | chr17: 7165909 | CLDN7.TSS200 | SCLC-A | F | chr17: 7164285-7166245 | Island |
| cg02925076 | chr7: 158251297 | PTPRN2.GENEBODY | SCLC-A | F | chr7: 158247888- | S_Shore |
| | | | | | 158249312 | |
| cg05379127 | chr19: 12885939 | HOOK2.GENEBODY | SCLC-A | F | chr19: 12885756- | Island |
| | | | | | 12886519 | |
| cg09176256 | chr10: 119293967 | EMX2OS.GENEBODY | SCLC-A | F | chr10: 119293917- | Island |
| | | | | | 119297675 | |
| cg11944076 | chr15: 51997390 | SCG3.GENEBODY | SCLC-A | R | | |
| cg17519949 | chr19: 15292440 | NOTCH3.GENEBODY | SCLC-A | R | chr19: 15292399- | Island |
| | | | | | 15292632 | |
| cg18099070 | chr7: 157845265 | PTPRN2.GENEBODY | SCLC-A | R | | |
| cg18777231 | chr20: 62669807 | NCRNA00176.GENEBODY | SCLC-A | F | chr20: 62669304- | Island |
| | | | | | 62670730 | |
| cg26425555 | chr5: 16616459 | FAM134B.GENEBODY | SCLC-A | F | chr5: 16616509- | N_Shore |
| | | | | | 16617428 | |
| cg26532826 | chr22: 31003127 | TCN2.TSS200 | SCLC-A | R | | |
| cg27269561 | chr16: 3072713 | HCFC1R1.UTR3 | SCLC-A | F | chr16: 3073686-3074443 | N_Shore |
| cg26147269 | chr3: 13520256 | HDAC11.TSS1500 | SCLC-A | F | chr3: 13520912- | N_Shore |
| | | | | | 13521319 | |
| cg14632140 | chr12: 16758112 | LMO3.UTR5 | SCLC-A | R | | |
| cg17107112 | chr6: 34021546 | GRM4.GENEBODY | SCLC-A | R | chr6: 34024201- | N_Shelf |
| | | | | | 34024457 | |
| cg19091386 | chr7: 157435297 | PTPRN2.GENEBODY | SCLC-A | F | | |
| cg04657700 | chr2: 223535084 | MOGAT1.TSS1500 | SCLC-A | F | chr2: 223536122- | N_Shore |
| | | | | | 223536565 | |
| cg09890699 | chr1: 54821853 | SSBP3.GENEBODY | SCLC-A | R | chr1: 54821990- | N_Shore |
| | | | | | 54822333 | |
| cg12132731 | chr1: 205313575 | KLHDC8A.UTR5 | SCLC-A | F | chr1: 205312493- | Island |
| | | | | | 205313576 | |
| cg14882082 | chr14: 100845427 | WDR25.UTR5 | SCLC-A | F | chr14: 100842339- | S_Shelf |
| | | | | | 100842931 | |
| cg16071713 | chr21: 36412456 | C21orf96.TSS1500 | SCLC-A | F | | |
| cg17956079 | chr11: 73358572 | PLEKHB1.TSS200 | SCLC-A | R | | |
| cg18831409 | chr6: 10585851 | GCNT2.GENEBODY | SCLC-A | F | | |
| cg23674344 | chr17: 48503163 | ACSF2.TSS1500 | SCLC-A | F | chr17: 48503056- | Island |
| | | | | | 48503887 | |
| cg00489219 | chr3: 40566141 | ZNF621.TSS1500 | SCLC-A | F | chr3: 40566129- | Island |
| | | | | | 40566689 | |
| cg01731355 | chr7: 158103582 | PTPRN2.GENEBODY | SCLC-A | F | chr7: 158103336- | Island |
| | | | | | 158103601 | |
| cg05116002 | chr3: 58151255 | FLNB.GENEBODY | SCLC-A | R | | |
| cg05264223 | chr15: 70126776 | C15orf50.TSS1500 | SCLC-A | F | | |
| cg05518803 | chr10: 124739553 | PSTK.TSS200 | SCLC-A | R | chr10: 124739792- | N_Shore |
| | | | | | 124740292 | |
| cg08230118 | chr6: 30181847 | TRIM26.TSS1500 | SCLC-A | R | chr6: 30180933- | Island |
| | | | | | 30182187 | |
| cg10451262 | chr8: 124284176 | ZHX1.UTR5 | SCLC-A | R | chr8: 124286209- | N_Shelf |
| | | | | | 124287666 | |
| cg12233379 | chr1: 151812524 | LOC100132111.GENEBODY | SCLC-A | F | chr1: 151812253- | Island |
| | | | | | 151812525 | |
| cg13275679 | chr17: 73151149 | HN1.TSS1500 | SCLC-A | R | chr17: 73149830- | S_Shore |
| | | | | | 73150885 | |
| cg16522557 | chr13: 111159111 | COL4A2.GENEBODY | SCLC-A | F | | |
| cg20427144 | chr7: 5518918 | FBXL18.UTR3 | SCLC-A | F | chr7: 5521368-5521615 | N_Shelf |
| cg20806345 | chr2: 182543706 | NEUROD1.UTR5 | SCLC-A | F | | |
| cg09565319 | chr6: 168963731 | SMOC2.GENEBODY | SCLC-A | R | | |
| cg02185402 | chr20: 400361 | RBCK1.GENEBODY | SCLC-A | F | chr20: 400244-400469 | Island |
| cg03721064 | chr7: 50535834 | DDC.GENEBODY | SCLC-A | R | chr7: 50535740- | Island |
| | | | | | 50535953 | |
| cg07908874 | chr10: 135123006 | ZNF511.GENEBODY | SCLC-A | R | chr10: 135122851- | Island |
| | | | | | 135123109 | |
| cg11785537 | chr12: 6484729 | SCNN1A.GENEBODY | SCLC-A | F | | |
| cg18011899 | chr7: 158124253 | PTPRN2.GENEBODY | SCLC-A | F | | |
| cg21913981 | chr12: 120151706 | MIR1178.TSS200 | SCLC-A | R | | |
| cg23967873 | chr10: 72977537 | UNC5B.GENEBODY | SCLC-A | R | chr10: 72977123- | Island |
| | | | | | 72977774 | |
| cg26363039 | chr12: 1609549 | LOC100292680.TSS200 | SCLC-A | R | | |
| cg27430662 | chr1: 109656708 | KIAA1324.UTR5 | SCLC-A | R | chr1: 109656095- | S_Shore |
| | | | | | 109656611 | |
| cg00870849 | chr10: 94003008 | CPEB3.UTR5 | SCLC-A | F | chr10: 93999131- | S_Shelf |
| | | | | | 94000015 | |
| cg05765011 | chr16: 4103225 | ADCY9.GENEBODY | SCLC-A | F | | |
| cg08521073 | chr12: 117314052 | HRK.UTR3 | SCLC-A | R | chr12: 117316389- | N_Shelf |
| | | | | | 117317611 | |
| cg12218991 | chr4: 152042934 | SH3D19.UTR3 | SCLC-A | F | | |
| cg14014950 | chr3: 64125334 | PRICKLE2.GENEBODY | SCLC-A | R | | |
| cg14220262 | chr1: 1114963 | TTLL10.GENEBODY | SCLC-A | F | chr1: 1115574-1116006 | N_Shore |
| cg18104606 | chr6: 70990361 | COL9A1.GENEBODY | SCLC-A | R | chr6: 70992040- | N_Shore |
| | | | | | 70992912 | |
| cg20417351 | chr17: 424358 | VPS53.GENEBODY | SCLC-A | R | | |
| cg01154445 | chr2: 206548171 | NRP2.GENEBODY | SCLC-A | F | chr2: 206549599- | N_Shore |
| | | | | | 206551316 | |
| cg01804664 | chr5: 149669414 | CAMK2A.TSS200 | SCLC-A | F | | |
| cg04569165 | chrX: 153688677 | PLXNA3.GENEBODY | SCLC-A | F | chrX: 153688592- | Island |
| | | | | | 153688860 | |
| cg04957757 | chr16: 70463218 | ST3GAL2.UTR5 | SCLC-A | R | | |
| cg07660671 | chr8: 10586967 | SOX7.GENEBODY | SCLC-A | F | chr8: 10587304- | N_Shore |
| | | | | | 10591109 | |
| cg09354317 | chr3: 52869307 | MUSTN1.TSS200 | SCLC-A | F | | |
| cg10724947 | chr15: 62359485 | C2CD4A.UTR5 | SCLC-A | F | chr15: 62359827- | N_Shore |
| | | | | | 62360995 | |
| cg11484283 | chr4: 159816181 | FNIP2.GENEBODY | SCLC-A | F | | |
| cg12271047 | chr7: 157387106 | PTPRN2.GENEBODY | SCLC-A | R | chr7: 157387053- | Island |
| | | | | | 157387322 | |
| cg17833037 | chr12: 123259751 | CCDC62.GENEBODY | SCLC-A | R | | |
| cg19563107 | chr14: 53418879 | FERMT2.TSS1500 | SCLC-A | R | chr14: 53417108- | S_Shore |
| | | | | | 53418339 | |
| cg20354580 | chr3: 128713146 | KIAA1257.TSS200 | SCLC-A | R | chr3: 128712404- | S_Shore |
| | | | | | 128713058 | |
| cg22110158 | chr11: 130036542 | ST14.GENEBODY | SCLC-A | F | | |
| cg22369288 | chr6: 138538817 | PBOV1.EXON1 | SCLC-A | R | | |
| cg22514229 | chr1: 94118054 | BCAR3.GENEBODY | SCLC-A | R | | |
| cg02954284 | chr22: 21367447 | MGC16703.GENEBODY | SCLC-A | R | chr22: 21368197- | N_Shore |
| | | | | | 21368771 | |
| cg04765195 | chr6: 36209753 | PNPLA1.TSS1500 | SCLC-A | F | | |
| cg08059112 | chr19: 2294887 | LINGO3.UTR5 | SCLC-A | R | chr19: 2294886-2295101 | Island |
| cg09508022 | chr6: 168971135 | SMOC2.GENEBODY | SCLC-A | F | chr6: 168972516- | N_Shore |
| | | | | | 168974109 | |
| cg09915444 | chr15: 62359275 | C2CD4A.EXON1 | SCLC-A | F | chr15: 62359827- | N_Shore |
| | | | | | 62360995 | |
| cg13859860 | chr10: 51569878 | NCOA4.UTR5 | SCLC-A | F | chr10: 51572198- | N_Shelf |
| | | | | | 51572620 | |
| cg14666908 | chr6: 31937758 | STK19.TSS1500 | SCLC-A | R | chr6: 31939730- | N_Shore |
| | | | | | 31940559 | |
| cg15744134 | chr15: 32933835 | SCG5.TSS200 | SCLC-A | R | | |
| cg16404364 | chr7: 1006746 | COX19.UTR3 | SCLC-A | F | chr7: 1007159-1007558 | N_Shore |
| cg19711579 | chr2: 182545562 | NEUROD1.TSS200 | SCLC-A | F | chr2: 182547873- | N_Shelf |
| | | | | | 182549177 | |
| cg20587332 | chr8: 40736768 | ZMAT4.UTR5 | SCLC-A | R | | |
| cg21276549 | chr13: 102568345 | FGF14.GENEBODY | SCLC-A | F | chr13: 102568425- | N_Shore |
| | | | | | 102569495 | |
| cg21390575 | chr4: 185747721 | ACSL1.TSS1500 | SCLC-A | R | chr4: 185746557- | Island |
| | | | | | 185747827 | |
| cg22976218 | chr2: 182544321 | NEUROD1.UTR5 | SCLC-A | R | chr2: 182547873- | N_Shelf |
| | | | | | 182549177 | |
| cg03569876 | chr5: 148908014 | CSNK1A1.GENEBODY | SCLC-A | R | | |
| cg03674563 | chr11: 422260 | ANO9.GENEBODY | SCLC-A | R | chr11: 420265-420975 | S_Shore |
| cg03905369 | chr2: 120824615 | EPB41L5.GENEBODY | SCLC-A | F | | |
| cg06374811 | chr6: 22099667 | FLJ22536.GENEBODY | SCLC-A | R | | |
| cg14795528 | chr7: 157451260 | PTPRN2.GENEBODY | SCLC-A | F | chr7: 157453774- | N_Shelf |
| | | | | | 157454208 | |
| cg19359218 | chr6: 30181936 | TRIM26.TSS1500 | SCLC-A | R | chr6: 30180933- | Island |
| | | | | | 30182187 | |
| cg20143111 | chr4: 10673188 | CLNK.UTR5 | SCLC-A | R | | |
| cg21163701 | chr8: 85165845 | RALYL.UTR5 | SCLC-A | F | | |
| cg26743132 | chr13: 111107905 | COL4A2.GENEBODY | SCLC-A | F | chr13: 111109139- | N_Shore |
| | | | | | 111109652 | |
| cg09918657 | chr16: 14397207 | MIR193B.TSS1500 | SCLC-A | R | chr16: 14395604- | S_Shore |
| | | | | | 14397075 | |
| cg19754622 | chr10: 134045578 | STK32C.GENEBODY | SCLC-A | F | chr10: 134041499- | S_Shelf |
| | | | | | 134041784 | |
| cg03769629 | chr6: 30181591 | TRIM26.TSS1500 | SCLC-A | R | chr6: 30180933- | Island |
| | | | | | 30182187 | |
| cg05640721 | chr15: 90437833 | AP3S2.TSS1500 | SCLC-A | F | chr15: 90437025- | S_Shore |
| | | | | | 90437602 | |
| cg06059849 | chr1: 109656930 | KIAA1324.EXON1 | SCLC-A | F | chr1: 109656095- | S_Shore |
| | | | | | 109656611 | |
| cg12609243 | chr3: 54155240 | CACNA2D3.TSS1500 | SCLC-A | R | chr3: 54155239- | Island |
| | | | | | 54157025 | |
| cg19744528 | chr7: 157553346 | PTPRN2.GENEBODY | SCLC-A | F | chr7: 157550547- | S_Shelf |
| | | | | | 157551025 | |
| cg22222281 | chr12: 6308758 | CD9.TSS1500 | SCLC-A | R | chr12: 6309152-6310392 | N_Shore |
| cg22740141 | chr12: 129454194 | GLT1D1.GENEBODY | SCLC-A | F | | |
| cg25191573 | chr3: 49037070 | P4HTM.GENEBODY | SCLC-A | R | | |
| cg25903363 | chr8: 21972479 | HR.UTR3 | SCLC-A | R | | |
| cg00321901 | chr8: 10512459 | RP1L1.EXON1 | SCLC-A | R | | |
| cg03695433 | chr17: 71332907 | SDK2.UTR3 | SCLC-A | F | | |
| cg04628438 | chr6: 10585889 | GCNT2.GENEBODY | SCLC-A | F | | |
| cg14981313 | chr1: 42847055 | RIMKLA.GENEBODY | SCLC-A | R | chr1: 42845978- | S_Shore |
| | | | | | 42846988 | |
| cg24408763 | chr6: 43446221 | TJAP1.UTR5 | SCLC-A | R | | |
| cg24603926 | chr12: 120151679 | MIR1178.TSS200 | SCLC-A | R | | |
| cg27098470 | chr22: 42828411 | NFAM1.TSS200 | SCLC-A | R | | |
| cg02102889 | chr4: 139164723 | SLC7A11.TSS1500 | SCLC-A | R | | |
| cg02910913 | chr17: 72968277 | C17orf28.GENEBODY | SCLC-A | R | chr17: 72968167- | Island |
| | | | | | 72969164 | |
| cg04176122 | chr11: 118779835 | BCL9L.GENEBODY | SCLC-A | F | chr11: 118781060- | N_Shore |
| | | | | | 118781732 | |
| cg06023257 | chr11: 78129396 | GAB2.TSS1500 | SCLC-A | F | chr11: 78127965- | Island |
| | | | | | 78129400 | |
| cg06233018 | chr21: 41552656 | DSCAM.GENEBODY | SCLC-A | F | chr21: 41554618- | N_Shore |
| | | | | | 41554959 | |
| cg15409237 | chr19: 51524249 | KLK10.TSS1500 | SCLC-A | F | chr19: 51522004- | S_Shore |
| | | | | | 51522803 | |
| cg17401435 | chr11: 63887426 | MACROD1.GENEBODY | SCLC-A | F | chr11: 63887355- | Island |
| | | | | | 63887600 | |
| cg19008877 | chr10: 78157854 | C10orf11.GENEBODY | SCLC-A | R | | |
| cg21275690 | chr16: 3072970 | HCFC1R1.GENEBODY | SCLC-A | F | chr16: 3073686-3074443 | N_Shore |
| cg26216243 | chr6: 49495283 | GLYATL3.UTR3 | SCLC-A | F | | |
| cg11286977 | chr6: 30181713 | TRIM26.TSS1500 | SCLC-A | R | chr6: 30180933- | Island |
| | | | | | 30182187 | |
| cg01027553 | chr7: 4037843 | SDK1.GENEBODY | SCLC-A | F | | |
| cg01155325 | chr15: 42719757 | ZFP106.GENEBODY | SCLC-A | R | | |
| cg06714480 | chr2: 182544307 | NEUROD1.UTR5 | SCLC-A | R | chr2: 182547873- | N_Shelf |
| | | | | | 182549177 | |
| cg07181952 | chr1: 1239999 | ACAP3.GENEBODY | SCLC-A | F | chr1: 1242400-1245185 | N_Shelf |
| cg09306186 | chr16: 73081722 | ZFHX3.EXON1 | SCLC-A | F | chr16: 73081590- | Island |
| | | | | | 73082565 | |
| cg10545682 | chr1: 151512625 | TUFT1.TSS200 | SCLC-A | F | chr1: 151512661- | N_Shore |
| | | | | | 151513199 | |
| cg10769178 | chr7: 157876300 | PTPRN2.GENEBODY | SCLC-A | R | | |
| cg20709008 | chr2: 182545934 | NEUROD1.TSS1500 | SCLC-A | F | chr2: 182547873- | N_Shore |
| | | | | | 182549177 | |
| cg24440468 | chr7: 73184642 | CLDN3.TSS200 | SCLC-A | R | chr7: 73183379- | Island |
| | | | | | 73185115 | |
| cg25356639 | chr3: 71349304 | FOXP1.UTR5 | SCLC-A | F | | |
| cg26824709 | chr5: 159740383 | CCNJL.TSS1500 | SCLC-A | R | chr5: 159738781- | S_Shore |
| | | | | | 159739913 | |
| cg01461199 | chr8: 121822473 | SNTB1.GENEBODY | SCLC-A | F | chr8: 121823534- | N_Shore |
| | | | | | 121824720 | |
| cg01788682 | chr3: 15382723 | SH3BP5.UTR5 | SCLC-A | F | | |
| cg03721293 | chr16: 90014378 | DEF8.TSS1500 | SCLC-A | F | chr16: 90014250- | Island |
| | | | | | 90014613 | |
| cg03893692 | chr5: 149669484 | CAMK2A.TSS200 | SCLC-A | F | | |
| cg07109923 | chr20: 35091317 | DLGAP4.GENEBODY | SCLC-A | R | chr20: 35089448- | S_Shore |
| | | | | | 35089998 | |
| cg11964773 | chr1: 14924077 | KIAA1026.TSS1500 | SCLC-A | F | chr1: 14924610- | N_Shore |
| | | | | | 14925993 | |
| cg13538006 | chr9: 95897313 | NINJ1.TSS1500 | SCLC-A | R | chr9: 95896007- | S_Shore |
| | | | | | 95897016 | |
| cg13818276 | chr22: 32437865 | SLC5A1.TSS1500 | SCLC-A | R | chr22: 32439046- | N_Shore |
| | | | | | 32439442 | |
| cg08530036 | chr1: 19496406 | UBR4.GENEBODY | SCLC-A | R | | |
| cg09388414 | chr7: 129414651 | MIR96.TSS200 | SCLC-A | R | chr7: 129418286- | N_Shelf |
| | | | | | 129422509 | |
| cg18124764 | chr7: 101558698 | CUX1.GENEBODY | SCLC-A | F | chr7: 101559402- | N_Shore |
| | | | | | 101559625 | |
| cg23406864 | chr22: 21053952 | TMEM191A.TSS1500 | SCLC-A | R | chr22: 21056258- | N_Shelf |
| | | | | | 21058793 | |
| cg23731742 | chr1: 879958 | NOC2L.UTR3 | SCLC-A | R | chr1: 875730-878363 | S_Shore |
| cg00336946 | chr13: 101186274 | A2LD1.TSS1500 | SCLC-A | F | chr13: 101184396- | S_Shore |
| | | | | | 101184848 | |
| cg00735329 | chr18: 74732603 | MBP.GENEBODY | SCLC-A | R | | |
| cg02375832 | chr11: 62437615 | C11orf48.UTR5 | SCLC-A | R | chr11: 62439047- | N_Shore |
| | | | | | 62439803 | |
| cg04757345 | chr4: 128918835 | C4orf29.GENEBODY | SCLC-A | F | | |
| cg11744304 | chr7: 100875822 | CLDN15.GENEBODY | SCLC-A | R | chr7: 100875555- | Island |
| | | | | | 100876212 | |
| cg16027376 | chr8: 144653975 | C8orf73.GENEBODY | SCLC-A | F | chr8: 144653774- | Island |
| | | | | | 144653976 | |
| cg19681793 | chr6: 169651645 | THBS2.UTR5 | SCLC-A | R | chr6: 169648546- | S_Shelf |
| | | | | | 169649047 | |
| cg21486510 | chr2: 7171998 | RNF144A.GENEBODY | SCLC-A | F | chr2: 7172296-7172512 | N_Shore |
| cg21761356 | chr12: 97892013 | RMST.GENEBODY | SCLC-A | F | | |
| cg22332321 | chr20: 62510260 | TPD52L2.GENEBODY | SCLC-A | R | chr20: 62509163- | S_Shore |
| | | | | | 62509369 | |
| cg22817352 | chr1: 204653629 | LRRN2.UTR5 | SCLC-A | F | chr1: 204654132- | N_Shore |
| | | | | | 204654885 | |
| cg17553353 | chr3: 111718565 | TAGLN3.GENEBODY | SCLC-A | R | | |
| cg02703870 | chr16: 82045140 | SDR42E1.TSS200 | SCLC-A | R | chr16: 82044886- | Island |
| | | | | | 82045306 | |
| cg03723730 | chr6: 34031694 | GRM4.GENEBODY | SCLC-A | F | | |
| cg13314260 | chr5: 141349631 | RNF14.UTR5 | SCLC-A | R | chr5: 141348531- | S_Shore |
| | | | | | 141349104 | |
| cg20660385 | chr19: 51526587 | KLK11.GENEBODY | SCLC-A | R | chr19: 51522004- | S_Shelf |
| | | | | | 51522803 | |
| cg20708173 | chr4: 7940426 | AFAP1.UTR5 | SCLC-A | R | chr4: 7940563-7941853 | N_Shore |
| cg23218957 | chr14: 59109856 | DACT1.GENEBODY | SCLC-A | R | chr14: 59113071- | N_Shelf |
| | | | | | 59113640 | |
| cg02641676 | chr8: 42397764 | C8orf40.UTR5 | SCLC-A | R | chr8: 42396235- | S_Shore |
| | | | | | 42397195 | |
| cg06829681 | chr11: 12867453 | TEAD1.GENEBODY | SCLC-A | F | | |
| cg09565670 | chr19: 39368408 | RINL.UTR5 | SCLC-A | R | | |
| cg14660446 | chr19: 48015621 | NAPA.GENEBODY | SCLC-A | F | chr19: 48017717- | N_Shelf |
| | | | | | 48018782 | |
| cg19296671 | chr11: 128562297 | FLI1.TSS200 | SCLC-A | R | chr11: 128562671- | N_Shore |
| | | | | | 128565011 | |
| cg02921634 | chr21: 43109546 | NCRNA00111.GENEBODY | SCLC-A | F | chr21: 43109349- | Island |
| | | | | | 43109687 | |
| cg03365751 | chr22: 42828386 | NFAM1.EXON1 | SCLC-A | F | | |
| cg06136185 | chr17: 79139087 | LOC388428.TSS1500 | SCLC-A | R | chr17: 79139501- | N_Shore |
| | | | | | 79140028 | |
| cg06963053 | chr2: 182545533 | NEUROD1.TSS200 | SCLC-A | F | chr2: 182547873- | N_Shelf |
| | | | | | 182549177 | |
| cg07915206 | chr15: 34260555 | AVEN.GENEBODY | SCLC-A | R | | |
| cg10806639 | chr13: 33780078 | STARD13.EXON1 | SCLC-A | R | | |
| cg18314778 | chr7: 157468321 | PTPRN2.GENEBODY | SCLC-A | F | | |
| cg18798446 | chr12: 103862780 | C12orf42.GENEBODY | SCLC-A | F | | |
| cg20970369 | chr1: 111744108 | DENND2D.TSS1500 | SCLC-A | F | chr1: 111746337- | N_Shelf |
| | | | | | 111747303 | |
| cg21244086 | chr15: 45479462 | SHF.GENEBODY | SCLC-A | F | chr15: 45479509- | N_Shore |
| | | | | | 45480498 | |
| cg10159951 | chr11: 35441881 | SLC1A2.TSS1500 | SCLC-A | F | chr11: 35440375- | Island |
| | | | | | 35441882 | |
| cg10614445 | chr2: 25526696 | DNMT3A.GENEBODY | SCLC-A | F | | |
| cg15873327 | chr19: 8398677 | KANK3.GENEBODY | SCLC-A | F | chr19: 8397958-8400461 | Island |
| cg22486214 | chr17: 78930466 | RPTOR.GENEBODY | SCLC-A | F | | |
| cg25043538 | chr11: 63887437 | MACROD1.GENEBODY | SCLC-A | R | chr11: 63887355- | Island |
| | | | | | 63887600 | |
| cg25960403 | chr4: 11370015 | MIR572.TSS1500 | SCLC-A | F | chr4: 11370229- | N_Shore |
| | | | | | 11370886 | |
| cg01670896 | chr8: 1933522 | KBTBD11.UTR5 | SCLC-A | F | | |
| cg01643250 | chr19: 15292485 | NOTCH3.GENEBODY | SCLC-A | R | chr19: 15292399- | Island |
| | | | | | 15292632 | |
| cg02959939 | chr21: 47813025 | PCNT.GENEBODY | SCLC-A | F | chr21: 47813251- | N_Shore |
| | | | | | 47813462 | |
| cg07533617 | chr11: 117624926 | DSCAML1.GENEBODY | SCLC-A | R | | |
| cg09824889 | chr3: 121311075 | FBXO40.TSS1500 | SCLC-A | R | | |
| cg10143807 | chr1: 155050924 | EFNA3.TSS1500 | SCLC-A | F | chr1: 155051155- | N_Shore |
| | | | | | 155055291 | |
| cg11445764 | chr5: 147773164 | FBXO38.UTR5 | SCLC-A | R | | |
| cg14765206 | chr10: 101744219 | DNMBP.UTR5 | SCLC-A | R | | |
| cg19239304 | chr2: 238621429 | LRRFIP1.GENEBODY | SCLC-A | R | | |
| cg20147326 | chr11: 33913538 | LMO2.EXON1 | SCLC-A | R | | |
| cg21798544 | chr11: 924646 | AP2A2.TSS1500 | SCLC-A | R | chr11: 925183-926083 | N_Shore |
| cg24918790 | chr22: 20785148 | SCARF2.GENEBODY | SCLC-A | F | chr22: 20783466- | Island |
| | | | | | 20786201 | |
| cg26142132 | chr17: 74463585 | AANAT.TSS200 | SCLC-A | R | | |
| cg03368643 | chr6: 30181600 | TRIM26.TSS1500 | SCLC-A | R | chr6: 30180933- | Island |
| | | | | | 30182187 | |
| cg07827420 | chr17: 75371764 | SEPT9.UTR5 | SCLC-A | F | chr17: 75368688- | S_Shore |
| | | | | | 75370506 | |
| cg15935860 | chr1: 201282499 | PKP1.GENEBODY | SCLC-A | F | | |
| cg26813796 | chr1: 225806065 | ENAH.GENEBODY | SCLC-A | R | | |
| cg19778462 | chr1: 41976072 | HIVEP3.UTR3 | SCLC-A | F | | |
| cg22149419 | chr17: 27253138 | PHF12.GENEBODY | SCLC-A | R | | |
| cg24994355 | chr10: 99445520 | AVPI1.UTR5 | SCLC-A | F | chr10: 99446696- | N_Shore |
| | | | | | 99447400 | |
| cg10965131 | chr7: 151078995 | WDR86.GENEBODY | SCLC-A | R | chr7: 151078462- | Island |
| | | | | | 151079536 | |
| cg12710538 | chr17: 27490340 | MYO18A.GENEBODY | SCLC-A | F | chr17: 27493203- | N_Shelf |
| | | | | | 27493880 | |
| cg13250203 | chr11: 1565798 | HCCA2.GENEBODY | SCLC-A | R | chr11: 1567940-1568907 | N_Shelf |
| cg13302670 | chr7: 44261293 | CAMK2B.GENEBODY | SCLC-A | F | chr7: 44259669- | S_Shore |
| | | | | | 44259923 | |
| cg17522866 | chr5: 147052374 | JAKMIP2.UTR5 | SCLC-A | F | | |
| cg25147139 | chr12: 49740718 | DNAJC22.TSS1500 | SCLC-A | R | chr12: 49738680- | Island |
| | | | | | 49740841 | |
| cg26245202 | chr1: 205602864 | ELK4.TSS1500 | SCLC-A | F | chr1: 205600493- | S_Shore |
| | | | | | 205601432 | |
| cg26317549 | chr15: 48497296 | SLC12A1.TSS1500 | SCLC-A | R | | |
| cg01474011 | chr2: 225435131 | CUL3.GENEBODY | SCLC-A | F | | |
| cg02737268 | chr20: 3780182 | CDC25B.GENEBODY | SCLC-A | F | chr20: 3776198-3776419 | S_Shelf |
| cg08549332 | chr7: 101558461 | CUX1.GENEBODY | SCLC-A | F | chr7: 101559402- | N_Shore |
| | | | | | 101559625 | |
| cg12108651 | chr7: 158193742 | PTPRN2.GENEBODY | SCLC-A | F | | |
| cg16935790 | chr17: 77043246 | C1QTNF1.GENEBODY | SCLC-A | R | chr17: 77043741- | N_Shore |
| | | | | | 77044489 | |
| cg21285555 | chr8: 52771466 | PCMTD1.GENEBODY | SCLC-A | R | | |
| cg26808293 | chr16: 3072207 | TNFRSF12A.UTR3 | SCLC-A | R | chr16: 3073686-3074443 | N_Shore |
| cg04956949 | chr7: 73119262 | STX1A.GENEBODY | SCLC-A | F | chr7: 73118500- | S_Shore |
| | | | | | 73118749 | |
| cg08519905 | chr12: 6308774 | CD9.TSS1500 | SCLC-A | R | chr12: 6309152-6310392 | N_Shore |
| cg09560636 | chr5: 43631579 | NNT.GENEBODY | SCLC-A | R | | |
| cg12400725 | chr1: 29240533 | EPB41.UTR5 | SCLC-A | R | chr1: 29240768- | N_Shore |
| | | | | | 29241969 | |
| cg15298719 | chr17: 7165255 | CLDN7.EXON1 | SCLC-A | R | chr17: 7164285-7166245 | Island |
| cg19397765 | chr6: 30181764 | TRIM26.TSS1500 | SCLC-A | R | chr6: 30180933- | Island |
| | | | | | 30182187 | |
| cg21585162 | chr11: 128566387 | FLI1.GENEBODY | SCLC-A | F | chr11: 128562671- | S_Shore |
| | | | | | 128565011 | |
| cg25004427 | chr5: 326523 | AHRR.GENEBODY | SCLC-A | R | chr5: 320788-323010 | S_Shelf |
| cg01609559 | chr6: 46138957 | ENPP5.TSS1500 | SCLC-A | R | | |
| cg08821738 | chr17: 77043179 | C1QTNF1.GENEBODY | SCLC-A | F | chr17: 77043741- | N_Shore |
| | | | | | 77044489 | |
| cg10690467 | chr12: 70047752 | BEST3.UTR3 | SCLC-A | R | | |
| cg14570838 | chr11: 128562353 | FLI1.TSS1500 | SCLC-A | F | chr11: 128562671- | N_Shore |
| | | | | | 128565011 | |
| cg17397159 | chr4: 720144 | PCGF3.UTR5 | SCLC-A | F | chr4: 716281-716977 | S_Shelf |
| cg18601229 | chr15: 42192297 | EHD4.UTR3 | SCLC-A | R | chr15: 42192913- | N_Shore |
| | | | | | 42193255 | |
| cg21445398 | chr2: 125425654 | CNTNAP5.GENEBODY | SCLC-A | F | | |
| cg00162859 | chr8: 131268256 | ASAP1.GENEBODY | SCLC-A | F | | |
| cg02659920 | chr11: 726335 | EPS8L2.GENEBODY | SCLC-A | F | chr11: 725596-726870 | Island |
| cg03852018 | chr2: 149634410 | KIF5C.GENEBODY | SCLC-A | F | chr2: 149632682- | S_Shore |
| | | | | | 149633882 | |
| cg04801402 | chr6: 46138912 | ENPP5.TSS200 | SCLC-A | F | | |
| cg08162476 | chr3: 13064001 | IQSEC1.GENEBODY | SCLC-A | R | | |
| cg08472795 | chr2: 129067253 | HS6ST1.GENEBODY | SCLC-A | F | | |
| cg02893260 | chr13: 30071603 | MTUS2.GENEBODY | SCLC-A | R | | |
| cg03508204 | chr2: 129057540 | HS6ST1.GENEBODY | SCLC-A | F | | |
| cg04176557 | chr11: 75902270 | WNT11.GENEBODY | SCLC-A | R | chr11: 75905717- | N_Shelf |
| | | | | | 75905918 | |
| cg05230392 | chr17: 55976522 | CUEDC1.UTR5 | SCLC-A | F | | |
| cg05912053 | chr15: 60919520 | RORA.GENEBODY | SCLC-A | R | | |
| cg08918013 | chr19: 55953951 | SHISA7.EXON1 | SCLC-A | R | chr19: 55953371- | Island |
| | | | | | 55954641 | |
| cg12758867 | chr11: 65354231 | EHBP1L1.GENEBODY | SCLC-A | F | chr11: 65352231- | S_Shore |
| | | | | | 65353134 | |
| cg13889934 | chr4: 86851399 | ARHGAP24.GENEBODY | SCLC-A | F | | |
| cg14454073 | chr14: 50159754 | KLHDC1.TSS200 | SCLC-A | F | chr14: 50159667- | Island |
| | | | | | 50160424 | |
| cg14475480 | chr17: 79655469 | HGS.GENEBODY | SCLC-A | R | chr17: 79655162- | Island |
| | | | | | 79655515 | |
| cg18041814 | chr6: 36802896 | CPNE5.GENEBODY | SCLC-A | F | chr6: 36806881- | N_Shelf |
| | | | | | 36807217 | |
| cg20735041 | chr3: 52869266 | MUSTN1.TSS200 | SCLC-A | R | | |
| cg25693302 | chr18: 55862678 | NEDD4L.UTR5 | SCLC-A | R | chr18: 55862653- | Island |
| | | | | | 55862873 | |
| cg04658856 | chr6: 16242468 | GMPR.GENEBODY | SCLC-A | R | chr6: 16238589- | S_Shelf |
| | | | | | 16239231 | |
| cg05004108 | chr1: 85931167 | DDAH1.TSS1500 | SCLC-A | F | chr1: 85929940- | Island |
| | | | | | 85931168 | |
| cg14986845 | chr6: 30182240 | TRIM26.TSS1500 | SCLC-A | R | chr6: 30180933- | S_Shore |
| | | | | | 30182187 | |
| cg15742605 | chr1: 879383 | SAMD11.GENEBODY | SCLC-A | F | chr1: 875730-878363 | S_Shore |
| cg19713140 | chr7: 157584994 | PTPRN2.GENEBODY | SCLC-A | F | chr7: 157586711- | N_Shore |
| | | | | | 157587112 | |
| cg20894936 | chr11: 1630266 | HCCA2.GENEBODY | SCLC-A | F | | |
| cg21060736 | chr15: 73618666 | HCN4.GENEBODY | SCLC-A | F | | |
| cg23417096 | chr8: 134250016 | NDRG1.UTR3 | SCLC-A | R | | |
| cg27104413 | chr3: 101044447 | SENP7.UTR3 | SCLC-A | R | | |
| cg21648069 | chr12: 1157718 | ERC1.GENEBODY | SCLC-A | F | | |
| cg00192882 | chr17: 19291120 | MFAP4.TSS1500 | SCLC-A | R | | |
| cg02838877 | chr13: 97873877 | MBNL2.TSS1500 | SCLC-A | R | | |
| cg03841136 | chr21: 43136835 | NCRNA00112.GENEBODY | SCLC-A | R | | |
| cg07701837 | chr2: 210516795 | MAP2.UTR5 | SCLC-A | F | | |
| cg09591406 | chr17: 3833873 | ATP2A3.GENEBODY | SCLC-A | R | | |
| cg09901529 | chr10: 75437967 | AGAP5.GENEBODY | SCLC-A | F | chr10: 75434361- | S_Shelf |
| | | | | | 75434584 | |
| cg25595636 | chr11: 61278148 | LRRC10B.EXON1 | SCLC-A | F | chr11: 61275825- | S_Shore |
| | | | | | 61277329 | |
| cg02716826 | chr9: 33447032 | SUGT1P1.GENEBODY | SCLC-A | R | chr9: 33447446- | N_Shore |
| | | | | | 33447824 | |
| cg07017242 | chr7: 128047441 | IMPDH1.GENEBODY | SCLC-A | F | chr7: 128045542- | S_Shore |
| | | | | | 128046298 | |
| cg13356427 | chr1: 6520354 | ESPN.UTR3 | SCLC-A | F | chr1: 6520584-6521955 | N_Shore |
| cg16944941 | chr11: 70391346 | SHANK2.GENEBODY | SCLC-A | F | | |
| cg18264519 | chr2: 97214296 | ARID5A.GENEBODY | SCLC-A | F | chr2: 97215788- | N_Shore |
| | | | | | 97216225 | |
| cg19626737 | chr15: 68633792 | ITGA11.GENEBODY | SCLC-A | F | | |
| cg21745110 | chr11: 19798742 | NAV2.GENEBODY | SCLC-A | R | chr11: 19798537- | Island |
| | | | | | 19798949 | |
| cg04098194 | chr1: 1241087 | ACAP3.GENEBODY | SCLC-A | R | chr1: 1242400-1245185 | N_Shore |
| cg07524505 | chr11: 44785843 | TSPAN18.TSS200 | SCLC-A | R | | |
| cg11808936 | chr4: 7894203 | AFAP1.UTR5 | SCLC-A | F | | |
| cg14157527 | chr9: 132937709 | FREQ.GENEBODY | SCLC-A | R | chr9: 132934213- | S_Shelf |
| | | | | | 132934483 | |
| cg16371032 | chr8: 95654541 | ESRP1.GENEBODY | SCLC-A | F | chr8: 95653898- | Island |
| | | | | | 95654733 | |
| cg11503687 | chr5: 138205434 | CTNNA1.GENEBODY | SCLC-A | F | | |
| cg12116749 | chr10: 21245178 | NEBL.GENEBODY | SCLC-A | R | | |
| cg02047211 | chr17: 78932697 | RPTOR.GENEBODY | SCLC-A | R | chr17: 78935043- | N_Shelf |
| | | | | | 78935307 | |
| cg02839802 | chr22: 38963969 | DMC1.GENEBODY | SCLC-A | F | chr22: 38965733- | N_Shore |
| | | | | | 38966196 | |
| cg04278162 | chr6: 30182172 | TRIM26.TSS1500 | SCLC-A | F | chr6: 30180933- | Island |
| | | | | | 30182187 | |
| cg19918429 | chr3: 122399781 | PARP14.EXON1 | SCLC-A | F | chr3: 122399655- | Island |
| | | | | | 122400190 | |
| cg00156118 | chr2: 85473750 | TCF7L1.GENEBODY | SCLC-A | R | | |
| cg04545079 | chr16: 67599924 | CTCF.UTR5 | SCLC-A | R | chr16: 67595868- | S_Shelf |
| | | | | | 67597554 | |
| cg07633435 | chr5: 152869009 | GRIA1.TSS1500 | SCLC-A | F | | |
| cg20129210 | chr13: 100547192 | CLYBL.UTR3 | SCLC-A | R | chr13: 100547172- | Island |
| | | | | | 100547431 | |
| cg08692676 | chr6: 134299594 | TBPL1.UTR5 | SCLC-A | R | | |
| cg09342325 | chr2: 220371699 | GMPPA.UTR3 | SCLC-A | F | | |
| cg10207240 | chr12: 122356781 | WDR66.UTR5 | SCLC-A | R | chr12: 122356315- | Island |
| | | | | | 122356853 | |
| cg11314042 | chr6: 143380966 | AIG1.TSS1500 | SCLC-A | F | chr6: 143381444- | N_Shore |
| | | | | | 143381918 | |
| cg14326805 | chr9: 71737756 | TJP2.UTR5 | SCLC-A | F | chr9: 71736074- | S_Shore |
| | | | | | 71737367 | |
| cg15018282 | chr6: 30181877 | TRIM26.TSS1500 | SCLC-A | R | chr6: 30180933- | Island |
| | | | | | 30182187 | |
| cg25887619 | chr7: 101558435 | CUX1.GENEBODY | SCLC-A | R | chr7: 101559402- | N_Shore |
| | | | | | 101559625 | |
| cg00970752 | chr1: 15711443 | FHAD1.GENEBODY | SCLC-A | R | | |
| cg02017200 | chr19: 49256361 | FUT1.UTR5 | SCLC-A | F | chr19: 49255778- | Island |
| | | | | | 49256495 | |
| cg03477240 | chr6: 20583779 | CDKAL1.GENEBODY | SCLC-A | F | | |
| cg03850117 | chr16: 87894334 | SLC7A5.GENEBODY | SCLC-A | R | chr16: 87894359- | N_Shore |
| | | | | | 87894566 | |
| cg15509065 | chr19: 36346314 | KIRREL2.TSS1500 | SCLC-A | F | chr19: 36347044- | N_Shore |
| | | | | | 36348101 | |
| cg16105461 | chr11: 61485853 | DAGLA.UTR5 | SCLC-A | F | chr11: 61488132- | N_Shelf |
| | | | | | 61488357 | |
| cg16933706 | chr6: 30181934 | TRIM26.TSS1500 | SCLC-A | R | chr6: 30180933- | Island |
| | | | | | 30182187 | |
| cg19139618 | chr17: 46504791 | SKAP1.GENEBODY | SCLC-A | R | chr17: 46507344- | N_Shelf |
| | | | | | 46507778 | |
| cg21804531 | chr14: 90419290 | C14orf143.GENEBODY | SCLC-A | F | chr14: 90420900- | N_Shore |
| | | | | | 90421175 | |
| cg23735214 | chr11: 77595033 | INTS4.GENEBODY | SCLC-A | F | | |
| cg26329692 | chr17: 73126757 | NT5C.GENEBODY | SCLC-A | R | chr17: 73127196- | N_Shore |
| | | | | | 73127995 | |
| cg03478739 | chr2: 175712381 | CHN1.GENEBODY | SCLC-A | F | | |
| cg04687437 | chr19: 511113 | C19orf20.GENEBODY | SCLC-A | F | chr19: 506526-507133 | S_Shelf |
| cg04828792 | chr21: 42733433 | MX2.TSS1500 | SCLC-A | F | | |
| cg08258650 | chr11: 35441900 | SLC1A2.TSS1500 | SCLC-A | F | chr11: 35440375- | S_Shore |
| | | | | | 35441882 | |
| cg08348709 | chr17: 37764802 | NEUROD2.TSS1500 | SCLC-A | F | chr17: 37761691- | S_Shore |
| | | | | | 37763551 | |
| cg09195271 | chr1: 20142497 | RNF186.TSS1500 | SCLC-A | R | | |
| cg10515731 | chr2: 220378996 | ACCN4.UTR5 | SCLC-A | F | chr2: 220377743- | S_Shore |
| | | | | | 220377946 | |
| cg18085176 | chr4: 7939940 | AFAP1.UTR5 | SCLC-A | F | chr4: 7940563-7941853 | N_Shore |
| cg22776856 | chr16: 3072572 | THOC6.TSS1500 | SCLC-A | R | chr16: 3073686-3074443 | N_Shore |
| cg25846061 | chr2: 236415673 | AGAP1.GENEBODY | SCLC-A | R | | |
| cg00785029 | chr21: 43491755 | UMODL1.GENEBODY | SCLC-A | R | | |
| cg01226742 | chr8: 37699923 | GPR124.UTR3 | SCLC-A | F | chr8: 37698846- | Island |
| | | | | | 37699931 | |
| cg04304450 | chr22: 43525431 | BIK.UTR3 | SCLC-A | F | | |
| cg04355435 | chr1: 43736290 | TMEM125.UTR5 | SCLC-A | F | chr1: 43738484- | N_Shelf |
| | | | | | 43738829 | |
| cg06804344 | chr3: 128778699 | GP9.TSS1500 | SCLC-A | F | | |
| cg06865992 | chr17: 37764503 | NEUROD2.TSS1500 | SCLC-A | R | chr17: 37761691- | S_Shore |
| | | | | | 37763551 | |
| cg08374799 | chr12: 53601050 | ITGB7.TSS200 | SCLC-A | R | | |
| cg08427782 | chr6: 166880989 | RPS6KA2.GENEBODY | SCLC-A | F | | |
| cg10483275 | chr5: 149669368 | CAMK2A.UTR5 | SCLC-A | F | | |
| cg14059959 | chr13: 111144447 | COL4A2.GENEBODY | SCLC-A | R | | |
| cg18498622 | chrX: 151810246 | GABRQ.GENEBODY | SCLC-A | F | chrX: 151806233- | S_Shelf |
| | | | | | 151806947 | |
| cg20192387 | chr6: 166856056 | RPS6KA2.GENEBODY | SCLC-A | R | | |
| cg21917090 | chr4: 77816250 | ANKRD56.UTR3 | SCLC-A | R | chr4: 77818015- | N_Shore |
| | | | | | 77819600 | |
| cg22359606 | chr2: 182545492 | NEUROD1.TSS200 | SCLC-A | F | chr2: 182547873- | N_Shelf |
| | | | | | 182549177 | |
| cg24554839 | chr1: 166124903 | MIR921.TSS1500 | SCLC-A | F | | |
| cg25788549 | chr7: 150786044 | AGAP3.GENEBODY | SCLC-A | F | chr7: 150783480- | S_Shore |
| | | | | | 150785040 | |
| cg06961289 | chr1: 156722410 | HDGF.TSS200 | SCLC-A | F | chr1: 156720747- | S_Shore |
| | | | | | 156722069 | |
| cg08671326 | chr3: 194967958 | C3orf21.GENEBODY | SCLC-A | F | | |
| cg09726982 | chr2: 220379016 | ACCN4.UTR5 | SCLC-A | F | chr2: 220377743- | S_Shore |
| | | | | | 220377946 | |
| cg13920460 | chr9: 71735701 | TJP2.TSS1500 | SCLC-A | R | chr9: 71736074- | N_Shore |
| | | | | | 71737367 | |
| cg19482025 | chr11: 128562245 | FLI1.TSS200 | SCLC-A | F | chr11: 128562671- | N_Shore |
| | | | | | 128565011 | |
| cg02746771 | chr10: 527872 | DIP2C.GENEBODY | SCLC-A | R | chr10: 525719-526041 | S_Shore |
| cg04907173 | chr11: 65060386 | POLA2.GENEBODY | SCLC-A | F | | |
| cg05124770 | chr22: 24578799 | SUSD2.GENEBODY | SCLC-A | F | chr22: 24581632- | N_Shelf |
| | | | | | 24582127 | |
| cg17204557 | chr2: 121223909 | LOC84931.GENEBODY | SCLC-A | R | | |
| cg19778003 | chr3: 111718062 | TAGLN3.EXON1 | SCLC-A | R | | |
| cg21177112 | chr12: 63193647 | PPM1H.GENEBODY | SCLC-A | F | chr12: 63195657- | N_Shelf |
| | | | | | 63195909 | |
| cg21781979 | chr13: 100547292 | CLYBL.UTR3 | SCLC-A | F | chr13: 100547172- | Island |
| | | | | | 100547431 | |
| cg21963703 | chr5: 140350766 | PCDHA7.GENEBODY | SCLC-A | R | chr5: 140346105- | 5.5 |
| | | | | | 140346931 | S_Shelf |
| cg22666115 | chr6: 30182186 | TRIM26.TSS1500 | SCLC-A | F | chr6: 30180933- | Island |
| | | | | | 30182187 | |
| cg23641264 | chr8: 143748641 | JRK.UTR5 | SCLC-A | F | chr8: 143750759- | N_Shelf |
| | | | | | 143751448 | |
| cg26064870 | chr8: 102944342 | NCALD.UTR5 | SCLC-A | R | | |
| cg03979241 | chr8: 21916824 | EPB49.EXON1 | SCLC-A | F | chr8: 21914275- | S_Shelf |
| | | | | | 21914525 | |
| cg07651914 | chr7: 100881986 | CLDN15.TSS1500 | SCLC-A | F | | |
| cg13077545 | chr17: 41832873 | SOST.GENEBODY | SCLC-A | R | chr17: 41832717- | Island |
| | | | | | 41833195 | |
| cg13974838 | chr9: 98221619 | PTCH1.GENEBODY | SCLC-A | F | | |
| cg15612436 | chr22: 43897902 | MPPED1.GENEBODY | SCLC-A | R | | |
| cg16222790 | chr8: 144521374 | ZC3H3.GENEBODY | SCLC-A | R | chr8: 144521358- | Island |
| | | | | | 144521608 | |
| cg17066349 | chr12: 120130646 | CIT.GENEBODY | SCLC-A | F | chr12: 120127973- | S_Shelf |
| | | | | | 120128291 | |
| cg18127395 | chr7: 95433546 | DYNC111.UTR5 | SCLC-A | F | | |
| cg25320780 | chr1: 110933345 | SLC16A4.UTR5 | SCLC-A | F | | |
| cg26549084 | chr3: 171759895 | FNDC3B.UTR5 | SCLC-A | F | chr3: 171757493- | S_Shore |
| | | | | | 171758724 | |
| cg27469783 | chr19: 3801404 | MATK.UTR5 | SCLC-A | F | chr19: 3801392-3801819 | Island |
| cg01877565 | chr7: 129414746 | MIR183.GENEBODY | SCLC-A | F | chr7: 129418286- | N_Shelf |
| | | | | | 129422509 | |
| cg03310469 | chr2: 45236672 | SIX2.TSS200 | SCLC-A | F | chr2: 45235511- | Island |
| | | | | | 45237792 | |
| cg04305228 | chr22: 18272333 | MICAL3.UTR3 | SCLC-A | R | chr22: 18275679- | N_Shelf |
| | | | | | 18275922 | |
| cg06433132 | chr19: 39662346 | PAK4.GENEBODY | SCLC-A | R | chr19: 39663600- | N_Shore |
| | | | | | 39663815 | |
| cg10832281 | chr11: 35499741 | PAMR1.GENEBODY | SCLC-A | F | | |
| cg14627862 | chr6: 169641232 | THBS2.GENEBODY | SCLC-A | F | chr6: 169641803- | N_Shore |
| | | | | | 169642020 | |
| cg19695266 | chr1: 1241672 | ACAP3.GENEBODY | SCLC-A | R | chr1: 1242400-1245185 | N_Shore |
| cg21151061 | chr12: 50498016 | GPD1.GENEBODY | SCLC-A | R | | |
| cg22352709 | chr3: 195446859 | MUC20.TSS1500 | SCLC-A | R | | |
| cg02619205 | chr9: 117157157 | AKNA.TSS1500 | SCLC-A | F | chr9: 117160139- | N_Shelf |
| | | | | | 117161064 | |
| cg03539717 | chr19: 13065086 | GADD45GIP1.GENEBODY | SCLC-A | R | chr19: 13067585- | N_Shelf |
| | | | | | 13068152 | |
| cg04007028 | chr17: 36735584 | SRCIN1.GENEBODY | SCLC-A | R | chr17: 36734659- | S_Shore |
| | | | | | 36735246 | |
| cg05499054 | chr8: 75148806 | JPH1.UTR3 | SCLC-A | R | | |
| cg07645286 | chr2: 173604634 | RAPGEF4.GENEBODY | SCLC-A | R | chr2: 173600281- | S_Shelf |
| | | | | | 173600970 | |
| cg11401784 | chr1: 16092264 | FBLIM1.GENEBODY | SCLC-A | R | | |
| cg14034852 | chr17: 7165010 | CLDN7.GENEBODY | SCLC-A | F | chr17: 7164285-7166245 | Island |
| cg17111837 | chr20: 62669757 | NCRNA00176.GENEBODY | SCLC-A | R | chr20: 62669304- | Island |
| | | | | | 62670730 | |
| cg20126106 | chr3: 111718135 | TAGLN3.EXON1 | SCLC-A | R | | |
| cg20491406 | chr1: 201693818 | NAV1.GENEBODY | SCLC-A | R | | |
| cg23377852 | chr5: 1339812 | CLPTM1L.GENEBODY | SCLC-A | F | chr5: 1339662-1340082 | Island |
| cg00912746 | chr1: 33350709 | HPCA.TSS1500 | SCLC-A | F | chr1: 33351686- | N_Shore |
| | | | | | 33352397 | |
| cg01557332 | chr6: 169641045 | THBS2.GENEBODY | SCLC-A | R | chr6: 169641803- | N_Shore |
| | | | | | 169642020 | |
| cg05304366 | chr15: 40226905 | EIF2AK4.GENEBODY | SCLC-A | F | chr15: 40226210- | S_Shore |
| | | | | | 40226631 | |
| cg08160706 | chr16: 11876111 | ZC3H7A.GENEBODY | SCLC-A | F | | |
| cg10716524 | chr11: 474509 | PTDSS2.GENEBODY | SCLC-A | R | chr11: 472781-473065 | S_Shore |
| cg11410436 | chr16: 52581609 | TOX3.UTR5 | SCLC-A | F | chr16: 52581293- | Island |
| | | | | | 52581610 | |
| cg19432362 | chr13: 46426263 | SIAH3.TSS1500 | SCLC-A | F | | |
| cg21191365 | chr14: 90529014 | KCNK13.GENEBODY | SCLC-A | F | chr14: 90526696- | S_Shore |
| | | | | | 90528951 | |
| cg03107310 | chr10: 86094757 | FAM190B.UTR5 | SCLC-A | F | | |
| cg06097994 | chr17: 77043171 | C1QTNF1.GENEBODY | SCLC-A | F | chr17: 77043741- | N_Shore |
| | | | | | 77044489 | |
| cg06923861 | chr11: 117355376 | DSCAML1.GENEBODY | SCLC-A | F | chr11: 117352691- | S_Shelf |
| | | | | | 117352961 | |
| cg13290371 | chr7: 5518071 | FBXL18.UTR3 | SCLC-A | R | chr7: 5521368-5521615 | N_Shelf |
| cg13948413 | chr1: 236687580 | LGALS8.UTR5 | SCLC-A | F | chr1: 236687071- | Island |
| | | | | | 236687608 | |
| cg14186816 | chr18: 55862763 | NEDD4L.UTR5 | SCLC-A | F | chr18: 55862653- | Island |
| | | | | | 55862873 | |
| cg15027294 | chr12: 1609537 | LOC100292680.TSS200 | SCLC-A | R | | |
| cg15962197 | chr1: 41704561 | SCMH1.UTR5 | SCLC-A | R | chr1: 41706957- | N_Shelf |
| | | | | | 41708327 | |
| cg17097293 | chr19: 3655825 | PIP5K1C.GENEBODY | SCLC-A | F | chr19: 3659641-3659949 | N_Shelf |
| cg18993457 | chr11: 441983 | ANO9.UTR5 | SCLC-A | R | | |
| cg19680850 | chr1: 37943106 | ZC3H12A.GENEBODY | SCLC-A | F | chr1: 37939723- | S_Shelf |
| | | | | | 37940650 | |
| cg21464975 | chr17: 60828260 | MARCH10.GENEBODY | SCLC-A | R | | |
| cg21832801 | chr11: 107579390 | SLN.UTR5 | SCLC-A | R | | |
| cg22019158 | chr1: 27926683 | AHDC1.UTR5 | SCLC-A | R | chr1: 27929705- | N_Shelf |
| | | | | | 27930866 | |
| cg05212460 | chr7: 2149814 | MAD1L1.GENEBODY | SCLC-A | R | | |
| cg08442823 | chr11: 45951803 | PHF21A.UTR3 | SCLC-A | F | | |
| cg11856932 | chr12: 53447059 | TENC1.UTR5 | SCLC-A | F | chr12: 53448008- | N_Shore |
| | | | | | 53448406 | |
| cg13002109 | chr3: 124041146 | KALRN.GENEBODY | SCLC-A | F | | |
| cg13160058 | chr8: 26243215 | BNIP3L.GENEBODY | SCLC-A | F | chr8: 26240290- | S_Shelf |
| | | | | | 26241017 | |
| cg14914552 | chr8: 97340188 | PTDSS1.GENEBODY | SCLC-A | R | | |
| cg15118447 | chr6: 159220343 | EZR.GENEBODY | SCLC-A | F | | |
| cg18335326 | chr6: 30653659 | KIAA1949.GENEBODY | SCLC-A | F | chr6: 30654392- | N_Shore |
| | | | | | 30654934 | |
| cg22962041 | chr17: 49235043 | NME1- | SCLC-A | R | chr17: 49230686- | S_Shelf |
| | | NME2.GENEBODY | | | 49231247 | |
| cg25619638 | chr2: 220310232 | SPEG.GENEBODY | SCLC-A | F | chr2: 220309637- | S_Shore |
| | | | | | 220309893 | |
| cg11076275 | chr1: 207069654 | IL24.TSS1500 | SCLC-A | F | | |
| cg13808641 | chr9: 96006533 | WNK2.GENEBODY | SCLC-A | R | chr9: 96009844- | N_Shelf |
| | | | | | 96010101 | |
| cg20666533 | chr13: 67714028 | PCDH9.GENEBODY | SCLC-A | F | | |
| cg22311015 | chr2: 241630042 | AQP12A.TSS1500 | SCLC-A | F | | |
| cg04671541 | chr3: 111717682 | TAGLN3.EXON1 | SCLC-A | F | | |
| cg05712010 | chr1: 230895283 | CAPN9.GENEBODY | SCLC-A | R | | |
| cg06488150 | chr7: 6476003 | DAGLB.GENEBODY | SCLC-A | F | | |
| cg07251887 | chr17: 73641809 | LOC100130933.TSS15 | SCLC-A | R | | |
| | | 00 | | | | |
| cg08969344 | chr6: 34031597 | GRM4.GENEBODY | SCLC-A | R | | |
| cg09537448 | chr7: 129414567 | MIR96.GENEBODY | SCLC-A | F | chr7: 129418286- | N_Shelf |
| | | | | | 129422509 | |
| cg18171392 | chr22: 24938255 | C22orf13.UTR3 | SCLC-A | F | | |
| cg20951539 | chr6: 36367819 | PXT1.GENEBODY | SCLC-A | R | | |
| cg21635706 | chr15: 69432565 | MIR548H4.GENEBODY | SCLC-A | R | | |
| cg22226592 | chr1: 109439213 | GPSM2.GENEBODY | SCLC-A | F | | |
| cg23375717 | chr11: 20883318 | NELL1.GENEBODY | SCLC-A | F | | |
| cg25670451 | chr12: 6487677 | SCNN1A.TSS1500 | SCLC-A | R | | |
| cg00010853 | chr6: 30653167 | KIAA1949.GENEBODY | SCLC-A | F | chr6: 30654392- | N_Shore |
| | | | | | 30654934 | |
| cg09901733 | chr17: 1731852 | RPA1.TSS1500 | SCLC-A | R | chr17: 1732834-1733612 | N_Shore |
| cg15245625 | chr18: 55862676 | NEDD4L.UTR5 | SCLC-A | R | chr18: 55862653- | Island |
| | | | | | 55862873 | |
| cg16640855 | chr2: 182543233 | NEUROD1.GENEBODY | SCLC-A | R | | |
| cg22047469 | chr1: 1110107 | TTLL10.UTR5 | SCLC-A | R | chr1: 1109314-1110145 | Island |
| cg22468751 | chr6: 136824489 | MAP7.GENEBODY | SCLC-A | F | | |
| cg12299554 | chr15: 94840953 | MCTP2.TSS1500 | SCLC-A | R | | |
| cg22169990 | chr7: 150786051 | AGAP3.GENEBODY | SCLC-A | F | chr7: 150783480- | S_Shore |
| | | | | | 150785040 | |
| cg23286081 | chr10: 125775778 | CHST15.GENEBODY | SCLC-A | R | | |
| cg24750887 | chr4: 89534723 | HERC3.GENEBODY | SCLC-A | R | | |
| cg12473285 | chr17: 41832975 | SOST.GENEBODY | SCLC-A | R | chr17: 41832717- | Island |
| | | | | | 41833195 | |
| cg00005437 | chr17: 18010740 | DRG2.UTR3 | SCLC-A | F | | |
| cg01132785 | chr4: 528175 | PIGG.GENEBODY | SCLC-A | F | chr4: 529166-529556 | N_Shore |
| cg02124184 | chr5: 72950691 | RGNEF.UTR5 | SCLC-A | F | | |
| cg04479757 | chr22: 26951319 | MIR548J.TSS200 | SCLC-A | R | | |
| cg05940240 | chr22: 41762302 | TEF.TSS1500 | SCLC-A | F | | |
| cg07096883 | chr7: 149979234 | ACTR3C.GENEBODY | SCLC-A | R | | |
| cg08196842 | chr14: 65016517 | C14orf50.TSS200 | SCLC-A | R | chr14: 65016516- | Island |
| | | | | | 65016909 | |
| cg08841552 | chr9: 140444378 | PNPLA7.GENEBODY | SCLC-A | F | chr9: 140445653- | N_Shore |
| | | | | | 140446891 | |
| cg10206969 | chr7: 5518392 | FBXL18.UTR3 | SCLC-A | F | chr7: 5521368-5521615 | N_Shelf |
| cg12941836 | chr3: 52869288 | MUSTN1.TSS200 | SCLC-A | F | | |
| cg19254163 | chr11: 60623782 | GPR44.TSS1500 | SCLC-A | R | chr11: 60619924- | S_Shelf |
| | | | | | 60621111 | |
| cg20465143 | chr8: 144653962 | C8orf73.GENEBODY | SCLC-A | F | chr8: 144653774- | Island |
| | | | | | 144653976 | |
| cg21563471 | chr2: 158184535 | ERMN.TSS1500 | SCLC-A | R | | |
| cg27412310 | chr11: 75146692 | GDPD5.GENEBODY | SCLC-A | R | | |
| cg07414392 | chr9: 71735648 | TJP2.TSS1500 | SCLC-A | R | chr9: 71736074- | N_Shore |
| | | | | | 71737367 | |
| cg07170253 | chr12: 48119903 | P11.TSS1500 | SCLC-A | R | | |
| cg13568213 | chr9: 136387235 | TMEM8C.GENEBODY | SCLC-A | F | | |
| cg14770958 | chr3: 111717793 | TAGLN3.EXON1 | SCLC-A | R | | |
| cg26068911 | chr5: 89967277 | GPR98.GENEBODY | SCLC-A | F | | |
| cg27281690 | chr2: 7171963 | RNF144A.GENEBODY | SCLC-A | F | chr2: 7172296-7172512 | N_Shore |
| cg02482603 | chr1: 174843754 | RABGAP1L.TSS1500 | SCLC-A | F | | |
| cg05380808 | chr1: 210820877 | HHAT.GENEBODY | SCLC-A | R | | |
| cg05593510 | chr5: 176249224 | UNC5A.GENEBODY | SCLC-A | R | chr5: 176244559- | S_Shelf |
| | | | | | 176245284 | |
| cg07505680 | chr8: 1933651 | KBTBD11.UTR5 | SCLC-A | F | | |
| cg08539179 | chr1: 227915948 | LOC100130093.TSS15 | SCLC-A | R | chr1: 227916425- | N_Shore |
| | | 00 | | | 227916736 | |
| cg10068572 | chr10: 35687029 | CCNY.GENEBODY | SCLC-A | F | | |
| cg10828127 | chr21: 41550814 | DSCAM.GENEBODY | SCLC-A | R | chr21: 41554618- | N_Shelf |
| | | | | | 41554959 | |
| cg11065015 | chr20: 327170 | NRSN2.TSS200 | SCLC-A | R | chr20: 327376-328977 | N_Shore |
| cg15554875 | chr2: 45236686 | SIX2.TSS200 | SCLC-A | F | chr2: 45235511- | Island |
| | | | | | 45237792 | |
| cg24092939 | chr12: 49181056 | ADCY6.UTR5 | SCLC-A | F | chr12: 49182421- | N_Shore |
| | | | | | 49182658 | |
| cg24321971 | chr11: 27406919 | LGR4.GENEBODY | SCLC-A | R | | |
| cg21887193 | chr7: 150786082 | AGAP3.GENEBODY | SCLC-A | F | chr7: 150783480- | S_Shore |
| | | | | | 150785040 | |
| cg02108198 | chr7: 99527876 | GJC3.TSS1500 | SCLC-A | F | | |
| cg05081953 | chr2: 158184543 | ERMN.TSS1500 | SCLC-A | F | | |
| cg09717545 | chr19: 917707 | KISS1R.EXON1 | SCLC-A | R | chr19: 917034-920787 | Island |
| cg11104313 | chr3: 111717534 | TAGLN3.TSS200 | SCLC-A | F | | |
| cg11672757 | chr2: 241080172 | OTOS.TSS200 | SCLC-A | R | | |
| cg14755690 | chr7: 128868265 | AHCYL2.GENEBODY | SCLC-A | R | chr7: 128864320- | S_Shelf |
| | | | | | 128865655 | |
| cg15169077 | chr6: 46139019 | ENPP5.TSS1500 | SCLC-A | F | | |
| cg17476271 | chr11: 128562408 | FLI1.TSS1500 | SCLC-A | F | chr11: 128562671- | N_Shore |
| | | | | | 128565011 | |
| cg24522085 | chr2: 169556419 | LASS6.GENEBODY | SCLC-A | F | | |
| cg04245402 | chr19: 751241 | C19orf21.UTR5 | SCLC-A | R | | |
| cg04785838 | chr6: 30181742 | TRIM26.TSS1500 | SCLC-A | F | chr6: 30180933- | Island |
| | | | | | 30182187 | |
| cg06400704 | chr1: 240256644 | FMN2.EXON1 | SCLC-A | F | chr1: 240254959- | Island |
| | | | | | 240257063 | |
| cg08200404 | chr15: 62359539 | C2CD4A.UTR5 | SCLC-A | F | chr15: 62359827- | N_Shore |
| | | | | | 62360995 | |
| cg08529481 | chr1: 36832173 | STK40.UTR5 | SCLC-A | F | | |
| cg10245608 | chr9: 92221282 | GADD45G.UTR3 | SCLC-A | R | chr9: 92219771- | Island |
| | | | | | 92221411 | |
| cg17838427 | chr1: 204653956 | LRRN2.UTR5 | SCLC-A | F | chr1: 204654132- | N_Shore |
| | | | | | 204654885 | |
| cg19786648 | chr17: 71824806 | C17orf54.TSS200 | SCLC-A | R | | |
| cg21068094 | chr18: 55862711 | NEDD4L.UTR5 | SCLC-A | R | chr18: 55862653- | Island |
| | | | | | 55862873 | |
| cg04994773 | chr1: 109656777 | KIAA1324.UTR5 | SCLC-A | R | chr1: 109656095- | S_Shore |
| | | | | | 109656611 | |
| cg06011067 | chr6: 110677913 | C6orf186.GENEBODY | SCLC-A | R | chr6: 110678854- | N_Shore |
| | | | | | 110679108 | |
| cg07562888 | chr10: 80883720 | ZMIZ1.UTR5 | SCLC-A | F | | |
| cg10598711 | chr5: 72470395 | TMEM174.UTR3 | SCLC-A | R | | |
| cg10976861 | chr2: 239149937 | HES6.TSS1500 | SCLC-A | F | chr2: 239147592- | S_Shore |
| | | | | | 239149900 | |
| cg12417176 | chr15: 55788778 | DYX1C1.GENEBODY | SCLC-A | F | chr15: 55790301- | N_Shore |
| | | | | | 55790754 | |
| cg13284574 | chr1: 6519923 | ESPN.GENEBODY | SCLC-A | F | chr1: 6520584-6521955 | N_Shore |
| cg16638385 | chr17: 41832753 | SOST.GENEBODY | SCLC-A | R | chr17: 41832717- | Island |
| | | | | | 41833195 | |
| cg21738316 | chrX: 153688775 | PLXNA3.GENEBODY | SCLC-A | F | chrX: 153688592- | Island |
| | | | | | 153688860 | |
| cg22618164 | chr12: 122356400 | WDR66.TSS200 | SCLC-A | F | chr12: 122356315- | Island |
| | | | | | 122356853 | |
| cg22626525 | chr1: 20008900 | TMCO4.UTR3 | SCLC-A | F | chr1: 20005510- | S_Shelf |
| | | | | | 20005865 | |
| cg23956760 | chr19: 19178708 | SLC25A42.UTR5 | SCLC-A | R | chr19: 19174262- | S_Shelf |
| | | | | | 19174995 | |
| cg24352878 | chr1: 8772712 | RERE.UTR5 | SCLC-A | R | | |
| cg27654966 | chr7: 101558400 | CUX1.GENEBODY | SCLC-A | R | chr7: 101559402- | N_Shore |
| | | | | | 101559625 | |
| cg04415689 | chr2: 242128250 | ANO7.GENEBODY | SCLC-A | F | | |
| cg07028869 | chr12: 49741164 | DNAJC22.UTR5 | SCLC-A | R | chr12: 49738680- | S_Shore |
| | | | | | 49740841 | |
| cg07354371 | chr1: 43736391 | TMEM125.UTR5 | SCLC-A | R | chr1: 43738484- | N_Shelf |
| | | | | | 43738829 | |
| cg07637741 | chr6: 159084426 | SYTL3.GENEBODY | SCLC-A | F | | |
| cg09833249 | chr12: 103312819 | PAH.TSS1500 | SCLC-A | R | chr12: 103311040- | S_Shore |
| | | | | | 103311253 | |
| cg11545871 | chr6: 33679895 | C6orf125.TSS1500 | SCLC-A | R | chr6: 33679158- | S_Shore |
| | | | | | 33679701 | |
| cg13714797 | chr9: 7103479 | KDM4C.GENEBODY | SCLC-A | R | | |
| cg22504528 | chr22: 26951369 | MIR548J.TSS200 | SCLC-A | F | | |
| cg23971638 | chr12: 741507 | NINJ2.GENEBODY | SCLC-A | R | | |
| cg24204017 | chr3: 57999988 | FLNB.GENEBODY | SCLC-A | F | | |
| cg26017492 | chr6: 166891729 | RPS6KA2.GENEBODY | SCLC-A | F | | |
| cg00369376 | chr2: 182542510 | NEUROD1.UTR3 | SCLC-A | F | | |
| cg02864954 | chr6: 154535553 | IPCEF1.GENEBODY | SCLC-A | R | | |
| cg03505501 | chr11: 115371789 | CADM1.GENEBODY | SCLC-A | R | chr11: 115373574- | N_Shore |
| | | | | | 115375881 | |
| cg04886060 | chr16: 82044788 | SDR42E1.UTR5 | SCLC-A | F | chr16: 82044886- | N_Shore |
| | | | | | 82045306 | |
| cg05161682 | chr13: 111107167 | COL4A2.GENEBODY | SCLC-A | R | chr13: 111109139- | N_Shore |
| | | | | | 111109652 | |
| cg09427605 | chr4: 146740968 | ZNF827.GENEBODY | SCLC-A | R | | |
| cg13396830 | chr6: 30181581 | TRIM26.TSS1500 | SCLC-A | R | chr6: 30180933- | Island |
| | | | | | 30182187 | |
| cg16427420 | chr17: 74526161 | CYGB.GENEBODY | SCLC-A | F | | |
| cg16931969 | chr4: 113985495 | ANK2.GENEBODY | SCLC-A | F | | |
| cg24652919 | chr16: 3073191 | HCFC1R1.GENEBODY | SCLC-A | R | chr16: 3073686-3074443 | N_Shore |
| cg26504422 | chr19: 44277951 | KCNN4.GENEBODY | SCLC-A | R | chr19: 44278273- | N_Shore |
| | | | | | 44278777 | |
| cg06537333 | chr16: 85643863 | KIAA0182.TSS1500 | SCLC-A | F | chr16: 85644089- | N_Shore |
| | | | | | 85644323 | |
| cg10156302 | chr15: 64202084 | DAPK2.GENEBODY | SCLC-A | F | | |
| cg13866253 | chr11: 77093001 | PAK1.GENEBODY | SCLC-A | R | | |
| cg14052044 | chr19: 751159 | C19orf21.EXON1 | SCLC-A | R | chr19: 746300-747161 | S_Shelf |
| cg14465207 | chr2: 10951250 | PDIA6.GENEBODY | SCLC-A | F | chr2: 10952402- | N_Shore |
| | | | | | 10953384 | |
| cg14695840 | chr9: 71737258 | TJP2.UTR5 | SCLC-A | R | chr9: 71736074- | Island |
| | | | | | 71737367 | |
| cg16832421 | chr2: 122229878 | CLASP1.GENEBODY | SCLC-A | F | | |
| cg18070562 | chr17: 8013617 | ALOXE3.GENEBODY | SCLC-A | R | chr17: 8013172-8013657 | Island |
| cg20592124 | chr2: 65290738 | CEP68.UTR5 | SCLC-A | R | | |
| cg25736482 | chr19: 54677232 | MBOAT7.UTR3 | SCLC-A | R | | |
| cg27417294 | chr10: 81904244 | PLAC9.GENEBODY | SCLC-A | R | | |
| cg27536453 | chr3: 169530417 | LRRC34.TSS200 | SCLC-A | F | chr3: 169529875- | Island |
| | | | | | 169530435 | |
| cg01248385 | chr13: 113636953 | MCF2L.GENEBODY | SCLC-A | R | chr13: 113635654- | S_Shore |
| | | | | | 113635899 | |
| cg03646740 | chr13: 114813015 | RASA3.GENEBODY | SCLC-A | R | chr13: 114813942- | N_Shore |
| | | | | | 114814457 | |
| cg08641990 | chr1: 54822503 | SSBP3.GENEBODY | SCLC-A | F | chr1: 54821990- | S_Shore |
| | | | | | 54822333 | |
| cg11482099 | chr11: 31833477 | PAX6.TSS1500 | SCLC-A | F | chr11: 31831620- | Island |
| | | | | | 31839038 | |
| cg14864994 | chr12: 6496214 | LTBR.GENEBODY | SCLC-A | F | chr12: 6492889-6493522 | S_Shelf |
| cg16681516 | chr19: 46297285 | DMWD.TSS1500 | SCLC-A | R | chr19: 46295855- | S_Shore |
| | | | | | 46296347 | |
| cg18998911 | chr3: 111718245 | TAGLN3.EXON1 | SCLC-A | R | | |
| cg20679581 | chr8: 10222777 | MSRA.GENEBODY | SCLC-A | F | | |
| cg24904739 | chr17: 77959804 | TBC1D16.GENEBODY | SCLC-A | R | | |
| cg25193885 | chr11: 70328867 | SHANK2.GENEBODY | SCLC-A | F | chr11: 70332266- | N_Shelf |
| | | | | | 70333413 | |
| cg03127370 | chr16: 2520192 | NTN3.TSS1500 | SCLC-A | R | chr16: 2521086-2525929 | N_Shore |
| cg12668122 | chr3: 133094221 | TMEM108.GENEBODY | SCLC-A | R | | |
| cg17749454 | chr17: 73101934 | SLC16A5.GENEBODY | SCLC-A | R | chr17: 73105229- | N_Shelf |
| | | | | | 73106667 | |
| cg21972567 | chr5: 158350708 | EBF1.GENEBODY | SCLC-A | F | | |
| cg25076197 | chr7: 104681440 | MLL5.GENEBODY | SCLC-A | F | | |
| cg26534460 | chr2: 215896959 | ABCA12.TSS200 | SCLC-A | F | | |
| cg15723222 | chr17: 59222476 | BCAS3.GENEBODY | SCLC-A | R | | |
| cg19420604 | chr17: 60828406 | MARCH10.GENEBODY | SCLC-A | F | | |
| cg02449373 | chr19: 49256123 | FUT1.UTR5 | SCLC-A | F | chr19: 49255778- | Island |
| | | | | | 49256495 | |
| cg03467001 | chr1: 183857649 | RGL1.GENEBODY | SCLC-A | R | | |
| cg03751944 | chr19: 1081936 | HMHA1.GENEBODY | SCLC-A | F | chr19: 1079682-1081937 | Island |
| cg04220881 | chr7: 152524814 | ACTR3B.GENEBODY | SCLC-A | F | | |
| cg05251389 | chr22: 43525330 | BIK.UTR3 | SCLC-A | F | | |
| cg13520715 | chr21: 43135854 | C21orf129.GENEBODY | SCLC-A | R | | |
| cg15559684 | chr3: 63428234 | SYNPR.GENEBODY | SCLC-A | R | | |
| cg19272238 | chr17: 76220898 | BIRC5.UTR3 | SCLC-A | F | | |
| cg20628733 | chr1: 3524459 | MEGF6.GENEBODY | SCLC-A | R | chr1: 3527619-3528078 | N_Shelf |
| cg26985681 | chr16: 15135397 | NTAN1.GENEBODY | SCLC-A | F | | |
| cg27274706 | chr11: 129685751 | TMEM45B.EXON1 | SCLC-A | R | chr11: 129685737- | Island |
| | | | | | 129686211 | |
| cg04930469 | chr17: 41832882 | SOST.GENEBODY | SCLC-A | R | chr17: 41832717- | Island |
| | | | | | 41833195 | |
| cg09853822 | chr17: 4712456 | PLD2.GENEBODY | SCLC-A | F | chr17: 4710014-4710960 | S_Shore |
| cg15460454 | chr7: 1974654 | MAD1L1.GENEBODY | SCLC-A | R | chr7: 1976258-1976464 | N_Shore |
| cg22532234 | chr19: 40229215 | CLC.TSS1500 | SCLC-A | R | | |
| cg04746649 | chr2: 169401301 | LASS6.GENEBODY | SCLC-A | R | | |
| cg09540626 | chr16: 2590751 | PDPK1.GENEBODY | SCLC-A | F | chr16: 2588034-2588556 | S_Shelf |
| cg22244768 | chr17: 55634551 | MSI2.GENEBODY | SCLC-A | F | | |
| cg26504421 | chr17: 71339361 | SDK2.GENEBODY | SCLC-A | R | | |
| cg06839900 | chr1: 2461900 | HES5.TSS1500 | SCLC-A | R | chr1: 2460760-2462010 | Island |
| cg03091070 | chr6: 169641288 | THBS2.GENEBODY | SCLC-A | R | chr6: 169641803- | N_Shore |
| | | | | | 169642020 | |
| cg14260458 | chr7: 97620123 | OCM2.TSS1500 | SCLC-A | R | | |
| cg17852245 | chr5: 153688187 | GALNT10.GENEBODY | SCLC-A | F | | |
| cg19974120 | chr21: 42839625 | TMPRSS2.GENEBODY | SCLC-A | F | | |
| cg20141509 | chr1: 10781431 | CASZ1.UTR5 | SCLC-A | F | | |
| cg25438492 | chr6: 33079692 | HLA-DPB2.TSS1500 | SCLC-A | F | | |
| cg26344024 | chr3: 71357195 | FOXP1.UTR5 | SCLC-A | R | | |
| cg27126442 | chr4: 106473806 | FLJ20184.UTR5 | SCLC-A | F | | |
| cg03018001 | chr19: 751098 | C19orf21.TSS200 | SCLC-A | R | chr19: 746300-747161 | S_Shelf |
| cg01132579 | chr17: 55588940 | MSI2.GENEBODY | SCLC-A | R | | |
| cg03029090 | chr15: 58812960 | LIPC.GENEBODY | SCLC-A | R | | |
| cg06694239 | chr7: 128807764 | TSPAN33.UTR3 | SCLC-A | F | chr7: 128809089- | N_Shore |
| | | | | | 128809393 | |
| cg08253704 | chr6: 30653242 | KIAA1949.GENEBODY | SCLC-A | R | chr6: 30654392- | N_Shore |
| | | | | | 30654934 | |
| cg08702745 | chr13: 111138063 | COL4A2.GENEBODY | SCLC-A | R | | |
| cg13793782 | chr9: 135995093 | RALGDS.GENEBODY | SCLC-A | F | chr9: 135995969- | N_Shore |
| | | | | | 135996954 | |
| cg15925940 | chr14: 105330630 | KIAA0284.TSS1500 | SCLC-A | R | chr14: 105330514- | Island |
| | | | | | 105332186 | |
| cg18612589 | chr17: 7165847 | CLDN7.TSS200 | SCLC-A | R | chr17: 7164285-7166245 | Island |
| cg20831413 | chr11: 72460551 | ARAP1.UTR5 | SCLC-A | R | chr11: 72463092- | N_Shelf |
| | | | | | 72463717 | |
| cg12248883 | chr11: 6219573 | OR52W1.TSS1500 | SCLC-A | F | | |
| cg00300637 | chr5: 319433 | AHRR.GENEBODY | SCLC-A | R | chr5: 320788-323010 | N_Shore |
| cg01661001 | chr20: 44439932 | UBE2C.TSS1500 | SCLC-A | F | chr20: 44440946- | N_Shore |
| | | | | | 44441655 | |
| cg02394998 | chr7: 157334850 | PTPRN2.GENEBODY | SCLC-A | R | | |
| cg04260891 | chr7: 75514575 | RHBDD2.GENEBODY | SCLC-A | F | | |
| cg12686273 | chr16: 3085867 | CCDC64B.TSS1500 | SCLC-A | F | chr16: 3086283-3086968 | N_Shore |
| cg13363823 | chr1: 40362907 | MYCL1.UTR3 | SCLC-A | F | chr1: 40366595- | N_Shelf |
| | | | | | 40368213 | |
| cg13401831 | chr22: 18593307 | TUBA8.TSS1500 | SCLC-A | R | chr22: 18593113- | Island |
| | | | | | 18594197 | |
| cg20234170 | chr11: 34460107 | CAT.TSS1500 | SCLC-A | F | chr11: 34460165- | N_Shore |
| | | | | | 34460857 | |
| cg22481263 | chr1: 906493 | PLEKHN1.GENEBODY | SCLC-A | R | chr1: 906296-906538 | Island |
| cg24688343 | chr17: 61229244 | TANC2.GENEBODY | SCLC-A | F | | |
| cg13844853 | chr1: 94062769 | BCAR3.GENEBODY | SCLC-A | F | | |
| cg20505457 | chr14: 69388953 | ACTN1.GENEBODY | SCLC-A | F | | |
| cg02923393 | chr16: 1543003 | TELO2.TSS1500 | SCLC-A | R | chr16: 1543098-1544426 | N_Shore |
| cg03021329 | chr3: 124554058 | ITGB5.GENEBODY | SCLC-A | F | | |
| cg07720856 | chr2: 232572668 | PTMA.TSS1500 | SCLC-A | F | chr2: 232571137- | Island |
| | | | | | 232575833 | |
| cg10708739 | chr17: 57465550 | YPEL2.GENEBODY | SCLC-A | F | | |
| cg12296860 | chr11: 46409428 | CHRM4.TSS1500 | SCLC-A | R | chr11: 46410921- | N_Shore |
| | | | | | 46414687 | |
| cg15602548 | chr2: 54786148 | SPTBN1.GENEBODY | SCLC-A | F | chr2: 54785026- | S_Shore |
| | | | | | 54785969 | |
| cg20925462 | chr3: 111717806 | TAGLN3.TSS200 | SCLC-A | R | | |
| cg21055933 | chr14: 64682702 | SYNE2.EXON1 | SCLC-A | F | | |
| cg24791666 | chr3: 39192215 | CSRNP1.UTR5 | SCLC-A | F | chr3: 39194241- | N_Shelf |
| | | | | | 39195359 | |
| cg00078299 | chr8: 124284379 | ZHX1.UTR5 | SCLC-A | R | chr8: 124286209- | N_Shore |
| | | | | | 124287666 | |
| cg00474798 | chr2: 84684489 | SUCLG1.GENEBODY | SCLC-A | R | chr2: 84686005- | N_Shore |
| | | | | | 84686751 | |
| cg00510447 | chr16: 3071608 | TNFRSF12A.GENEBODY | SCLC-A | F | chr16: 3070095-3071305 | S_Shore |
| cg00959749 | chr1: 54302681 | TMEM48.GENEBODY | SCLC-A | F | chr1: 54303758- | N_Shore |
| | | | | | 54304262 | |
| cg01267165 | chr16: 11681599 | LITAF.TSS1500 | SCLC-A | F | chr16: 11680008- | S_Shore |
| | | | | | 11681275 | |
| cg02405813 | chr6: 83072522 | TPBG.TSS1500 | SCLC-A | F | chr6: 83073416- | N_Shore |
| | | | | | 83075319 | |
| cg06665622 | chr6: 46293571 | RCAN2.TSS200 | SCLC-A | F | | |
| cg07460919 | chr3: 183980626 | ECE2.GENEBODY | SCLC-A | R | chr3: 183977548- | Island |
| | | | | | 183980633 | |
| cg08869273 | chr11: 413594 | SIGIRR.UTR5 | SCLC-A | R | chr11: 416419-417650 | N_Shelf |
| cg09238199 | chr13: 111097777 | COL4A2.GENEBODY | SCLC-A | F | | |
| cg09333109 | chr19: 51532580 | KLK11.TSS1500 | SCLC-A | F | chr19: 51535218- | N_Shelf |
| | | | | | 51535475 | |
| cg10813585 | chr1: 114305956 | RSBN1.UTR3 | SCLC-A | R | chr1: 114301514- | S_Shelf |
| | | | | | 114302272 | |
| cg11243251 | chr6: 36209708 | PNPLA1.TSS1500 | SCLC-A | R | | |
| cg14194715 | chr13: 32651552 | FRY.GENEBODY | SCLC-A | F | | |
| cg01262667 | chr19: 19385393 | TM6SF2.TSS1500 | SCLC-A | F | chr19: 19383703- | S_Shore |
| | | | | | 19384283 | |
| cg03581459 | chr14: 105780137 | PACS2.TSS1500 | SCLC-A | F | chr14: 105780339- | N_Shore |
| | | | | | 105782027 | |
| cg04961866 | chr16: 3085686 | CCDC64B.TSS200 | SCLC-A | R | chr16: 3086283-3086968 | N_Shore |
| cg05263838 | chr2: 227659406 | IRS1.UTR3 | SCLC-A | F | chr2: 227662082- | N_Shelf |
| | | | | | 227664912 | |
| cg05962028 | chr12: 214000 | IQSEC3.GENEBODY | SCLC-A | F | chr12: 213763-214227 | Island |
| cg11908131 | chr2: 106685045 | C2orf40.GENEBODY | SCLC-A | R | chr2: 106681982- | S_Shelf |
| | | | | | 106682403 | |
| cg14245347 | chr6: 29856426 | HLA-H.GENEBODY | SCLC-A | R | chr6: 29855295- | Island |
| | | | | | 29856565 | |
| cg14377791 | chr19: 40169912 | LOC400696.TSS200 | SCLC-A | F | | |
| cg16081441 | chr17: 74463560 | AANAT.TSS200 | SCLC-A | R | | |
| cg17889402 | chr18: 55862872 | NEDD4L.UTR5 | SCLC-A | F | chr18: 55862653- | Island |
| | | | | | 55862873 | |
| cg20169296 | chr10: 103367727 | RP11- | SCLC-A | R | | |
| | | 529I cg0076479610.4.GENEBODY | | | | |
| cg05554594 | chr17: 80584498 | WDR45L.GENEBODY | SCLC-A | F | | |
| cg06915226 | chr2: 70960959 | ADD2.UTR5 | SCLC-A | F | | |
| cg07121199 | chr1: 205537752 | MFSD4.TSS1500 | SCLC-A | R | chr1: 205537751- | Island |
| | | | | | 205538443 | |
| cg16929041 | chr17: 26648951 | TMEM97.GENEBODY | SCLC-A | F | chr17: 26645290- | S_Shelf |
| | | | | | 26645614 | |
| cg21521758 | chr7: 150036287 | RARRES2.GENEBODY | SCLC-A | F | chr7: 150037459- | N_Shore |
| | | | | | 150039031 | |
| cg00282244 | chr7: 100876136 | CLDN15.GENEBODY | SCLC-A | F | chr7: 100875555- | Island |
| | | | | | 100876212 | |
| cg01062519 | chr17: 34122169 | MMP28.GENEBODY | SCLC-A | F | chr17: 34122024- | Island |
| | | | | | 34122801 | |
| cg01796143 | chr1: 236687575 | LGALS8.UTR5 | SCLC-A | R | chr1: 236687071- | Island |
| | | | | | 236687608 | |
| cg01897496 | chr2: 182545209 | NEUROD1.EXON1 | SCLC-A | R | chr2: 182547873- | N_Shelf |
| | | | | | 182549177 | |
| cg02445907 | chr6: 36294404 | C6orf222.GENEBODY | SCLC-A | F | | |
| cg10351284 | chr8: 125617225 | MTSS1.GENEBODY | SCLC-A | F | | |
| cg12778007 | chr14: 72041267 | SIPA1L1.UTR5 | SCLC-A | F | | |
| cg13302994 | chr2: 210714233 | UNC80.GENEBODY | SCLC-A | F | | |
| cg16341266 | chr7: 147256367 | MIR54814.TSS1500 | SCLC-A | F | | |
| cg17265693 | chr17: 7166490 | CLDN7.TSS1500 | SCLC-A | F | chr17: 7164285-7166245 | S_Shore |
| cg17956387 | chr1: 208418015 | PLXNA2.TSS1500 | SCLC-A | R | chr1: 208416489- | Island |
| | | | | | 208418016 | |
| cg18319790 | chr14: 100788777 | C14orf68.TSS1500 | SCLC-A | R | | |
| cg21719937 | chr12: 5564478 | NTF3.GENEBODY | SCLC-A | R | | |
| cg21740204 | chr9: 72873544 | SMC5.TSS1500 | SCLC-A | F | chr9: 72873689- | N_Shore |
| | | | | | 72874264 | |
| cg24657047 | chr15: 77317370 | PSTPIP1.GENEBODY | SCLC-A | R | chr15: 77320150- | N_Shelf |
| | | | | | 77320456 | |
| cg26037504 | chr12: 56113808 | RDH5.TSS1500 | SCLC-A | F | chr12: 56109798- | S_Shelf |
| | | | | | 56110298 | |
| cg26782539 | chr10: 134061662 | STK32C.GENEBODY | SCLC-A | F | | |
| cg02010776 | chr16: 18938091 | SMG1.TSS1500 | SCLC-A | R | chr16: 18937030- | Island |
| | | | | | 18938126 | |
| cg03920522 | chr7: 94047477 | COL1A2.GENEBODY | SCLC-A | R | | |
| cg08946575 | chr1: 85931072 | DDAH1.TSS200 | SCLC-A | F | chr1: 85929940- | Island |
| | | | | | 85931168 | |
| cg10039567 | chr1: 112395111 | KCND3.GENEBODY | SCLC-A | R | | |
| cg10316635 | chr1: 62208645 | INADL.UTR5 | SCLC-A | R | chr1: 62207827- | Island |
| | | | | | 62208818 | |
| cg12930882 | chr5: 37825585 | GDNF.GENEBODY | SCLC-A | R | | |
| cg13973639 | chr6: 42858710 | C6orf226.TSS200 | SCLC-A | R | chr6: 42858169- | Island |
| | | | | | 42858960 | |
| cg15653828 | chr6: 34513187 | SPDEF.UTR5 | SCLC-A | F | chr6: 34512063- | S_Shore |
| | | | | | 34512266 | |
| cg19153095 | chr8: 106811143 | ZFPM2.GENEBODY | SCLC-A | F | | |
| cg26335620 | chr19: 48215149 | EHD2.TSS1500 | SCLC-A | F | chr19: 48216486- | N_Shore |
| | | | | | 48216837 | |
| cg11356571 | chr19: 48219924 | EHD2.GENEBODY | SCLC-A | R | chr19: 48219831- | Island |
| | | | | | 48220298 | |
| cg02531277 | chr7: 101558597 | CUX1.GENEBODY | SCLC-A | R | chr7: 101559402- | N_Shore |
| | | | | | 101559625 | |
| cg02565999 | chr13: 95003984 | GPC6.GENEBODY | SCLC-A | F | | |
| cg03036777 | chr1: 33648819 | TRIM62.TSS1500 | SCLC-A | F | chr1: 33646613- | S_Shore |
| | | | | | 33647878 | |
| cg03469805 | chr14: 105330648 | KIAA0284.TSS1500 | SCLC-A | F | chr14: 105330514- | Island |
| | | | | | 105332186 | |
| cg03621100 | chr12: 104322919 | LOC253724.GENEBODY | SCLC-A | F | chr12: 104323017- | N_Shore |
| | | | | | 104324811 | |
| cg04608356 | chr1: 27668433 | SYTL1.TSS200 | SCLC-A | R | | |
| cg06644669 | chr6: 30653799 | KIAA1949.UTR5 | SCLC-A | R | chr6: 30654392- | N_Shore |
| | | | | | 30654934 | |
| cg08706567 | chr1: 43814983 | MPL.GENEBODY | SCLC-A | F | chr1: 43814305- | Island |
| | | | | | 43815277 | |
| cg08905114 | chr9: 95897015 | NINJ1.TSS1500 | SCLC-A | R | chr9: 95896007- | Island |
| | | | | | 95897016 | |
| cg09702010 | chr19: 51531393 | KLK11.TSS1500 | SCLC-A | F | chr19: 51535218- | N_Shelf |
| | | | | | 51535475 | |
| cg09865760 | chr12: 49739893 | DNAJC22.TSS1500 | SCLC-A | R | chr12: 49738680- | Island |
| | | | | | 49740841 | |
| cg13264672 | chr7: 39993440 | CDK13.GENEBODY | SCLC-A | F | chr7: 39988922- | S_Shore |
| | | | | | 39991612 | |
| cg14607161 | chr6: 32084298 | ATF6B.GENEBODY | SCLC-A | R | | |
| cg17141902 | chr9: 95897216 | NINJ1.TSS1500 | SCLC-A | F | chr9: 95896007- | S_Shore |
| | | | | | 95897016 | |
| cg26681687 | chr4: 77874155 | SEPT11.GENEBODY | SCLC-A | F | chr4: 77869440- | S_Shelf |
| | | | | | 77872122 | |
| cg02512352 | chr7: 151137048 | CRYGN.EXON1 | SCLC-A | F | chr7: 151136933- | Island |
| | | | | | 151137273 | |
| cg05098417 | chr11: 64067858 | C11orf20.TSS200 | SCLC-A | F | chr11: 64066757- | Island |
| | | | | | 64068741 | |
| cg05155047 | chr5: 168719 | PLEKHG4B.GENEBODY | SCLC-A | F | chr5: 169578-169798 | N_Shore |
| cg06740893 | chr1: 12251869 | TNFRSF1B.GENEBODY | SCLC-A | R | chr1: 12251777- | Island |
| | | | | | 12251985 | |
| cg07065666 | chr6: 167034653 | RPS6KA2.GENEBODY | SCLC-A | R | chr6: 167032330- | S_Shelf |
| | | | | | 167032582 | |
| cg10754794 | chr10: 93916288 | CPEB3.GENEBODY | SCLC-A | F | | |
| cg12934258 | chr6: 159084599 | SYTL3.GENEBODY | SCLC-A | F | | |
| cg15744692 | chr15: 45671195 | LOC145663.GENEBODY | SCLC-A | F | chr15: 45669970- | S_Shore |
| | | | | | 45671029 | |
| cg21604615 | chr1: 27668610 | SYTL1.EXON1 | SCLC-A | F | | |
| cg00955867 | chr1: 160337038 | NHLH1.EXON1 | SCLC-A | R | chr1: 160340604- | N_Shelf |
| | | | | | 160340843 | |
| cg01470599 | chr9: 38422794 | IGFBPL1.GENEBODY | SCLC-A | F | chr9: 38423947- | N_Shore |
| | | | | | 38424584 | |
| cg01562385 | chr19: 10698098 | AP1M2.TSS200 | SCLC-A | R | chr19: 10697786- | Island |
| | | | | | 10698099 | |
| cg06508379 | chr19: 19104064 | SFRS14.UTR3 | SCLC-A | R | | |
| cg07491702 | chr15: 43030817 | CDAN1.TSS1500 | SCLC-A | R | chr15: 43028413- | S_Shore |
| | | | | | 43029346 | |
| cg08673532 | chr16: 87422913 | FBXO31.GENEBODY | SCLC-A | F | chr16: 87425137- | N_Shelf |
| | | | | | 87426677 | |
| cg16320329 | chr3: 45981161 | FYCO1.GENEBODY | SCLC-A | F | | |
| cg16321266 | chr7: 129414894 | MIR183.TSS200 | SCLC-A | F | chr7: 129418286- | N_Shelf |
| | | | | | 129422509 | |
| cg17033622 | chr11: 128568943 | FLI1.GENEBODY | SCLC-A | R | chr11: 128562671- | S_Shelf |
| | | | | | 128565011 | |
| cg17420360 | chr12: 49740840 | DNAJC22.TSS200 | SCLC-A | F | chr12: 49738680- | Island |
| | | | | | 49740841 | |
| cg18911861 | chr6: 42858743 | C6orf226.TSS200 | SCLC-A | R | chr6: 42858169- | Island |
| | | | | | 42858960 | |
| cg19599803 | chr1: 156722676 | HDGF.TSS1500 | SCLC-A | F | chr1: 156720747- | S_Shore |
| | | | | | 156722069 | |
| cg22791543 | chr5: 127416532 | FLJ33630.GENEBODY | SCLC-A | R | chr5: 127418594- | N_Shelf |
| | | | | | 127420512 | |
| cg26020521 | chr11: 8680436 | TRIM66.TSS200 | SCLC-A | F | | |
| cg26877508 | chr15: 71495324 | THSD4.GENEBODY | SCLC-A | R | | |
| cg27360567 | chr3: 49941613 | MST1R.TSS1500 | SCLC-A | F | chr3: 49940099- | S_Shore |
| | | | | | 49940431 | |
| cg00764796 | chr16: 433504 | LOC100134368.GENEBODY | SCLC-A | R | chr16: 431189-432810 | S_Shore |
| cg05305327 | chr20: 32156436 | CBFA2T2.GENEBODY | SCLC-A | R | | |
| cg08843859 | chr1: 184566345 | C1orf21.GENEBODY | SCLC-A | F | | |
| cg09027133 | chr1: 25945042 | MAN1C1.GENEBODY | SCLC-A | F | chr1: 25942661- | S_Shore |
| | | | | | 25943191 | |
| cg12145018 | chr14: 103912507 | MARK3.GENEBODY | SCLC-A | F | | |
| cg14087413 | chr9: 35848330 | TMEM8B.GENEBODY | SCLC-A | R | chr9: 35846310- | S_Shore |
| | | | | | 35846638 | |
| cg18449361 | chr6: 2689154 | MYLK4.GENEBODY | SCLC-A | R | chr6: 2685400-2685625 | S_Shelf |
| cg22393461 | chr1: 10010658 | NMNAT1.UTR5 | SCLC-A | F | | |
| cg23365482 | chr15: 40764195 | CHST14.EXON1 | SCLC-A | F | chr15: 40763162- | Island |
| | | | | | 40764196 | |
| cg22572454 | chr17: 74463767 | AANAT.GENEBODY | SCLC-A | F | | |
| cg00034154 | chr19: 10337163 | S1PR2.UTR5 | SCLC-A | F | chr19: 10334618- | S_Shore |
| | | | | | 10335511 | |
| cg03738134 | chr8: 144653855 | C8orf73.GENEBODY | SCLC-A | R | chr8: 144653774- | Island |
| | | | | | 144653976 | |
| cg04656424 | chr16: 88839844 | FAM38A.GENEBODY | SCLC-A | F | chr16: 88838112- | S_Shore |
| | | | | | 88838459 | |
| cg06561646 | chr17: 35873369 | DUSP14.UTR3 | SCLC-A | F | chr17: 35872722- | S_Shore |
| | | | | | 35873026 | |
| cg07119973 | chr17: 43109221 | DCAKD.GENEBODY | SCLC-A | R | chr17: 43111525- | N_Shelf |
| | | | | | 43111732 | |
| cg07920930 | chr7: 158381884 | PTPRN2.TSS1500 | SCLC-A | R | chr7: 158379328- | S_Shore |
| | | | | | 158381221 | |
| cg08637583 | chr15: 44828465 | EIF3J.TSS1500 | SCLC-A | R | chr15: 44828637- | N_Shore |
| | | | | | 44829949 | |
| cg14453201 | chr5: 320888 | AHRR.GENEBODY | SCLC-A | R | chr5: 320788-323010 | Island |
| cg15507486 | chr19: 46627896 | IGFL3.EXON1 | SCLC-A | F | | |
| cg17236870 | chr4: 94122883 | GRID2.GENEBODY | SCLC-A | F | | |
| cg21093368 | chr17: 8055834 | PER1.TSS200 | SCLC-A | F | chr17: 8054550-8055835 | Island |
| cg26477169 | chr17: 75196864 | SEC14L1.GENEBODY | SCLC-A | R | | |
| cg26692003 | chr3: 13063165 | IQSEC1.GENEBODY | SCLC-A | R | | |
| cg27262398 | chr11: 107578793 | SLN.UTR5 | SCLC-A | R | | |
| cg27559097 | chr2: 144987743 | GTDC1.UTR5 | SCLC-A | F | | |
| cg12817389 | chr8: 62602522 | ASPH.GENEBODY | SCLC-A | R | | |
| cg18407095 | chr2: 26846581 | CIB4.GENEBODY | SCLC-A | R | | |
| cg08439728 | chr6: 31097073 | PSORS1C1.UTR5 | SCLC-A | F | | |
| cg00225157 | chr6: 33173038 | HSD17B8.GENEBODY | SCLC-A | F | chr6: 33172289- | S_Shore |
| | | | | | 33172823 | |
| cg00648184 | chr17: 60828157 | MARCH10.GENEBODY | SCLC-A | R | | |
| cg01930441 | chr16: 21289268 | CRYM.GENEBODY | SCLC-A | F | chr16: 21289419- | N_Shore |
| | | | | | 21289819 | |
| cg03035849 | chr6: 91003200 | BACH2.UTR5 | SCLC-A | R | chr6: 91004795- | N_Shore |
| | | | | | 91006944 | |
| cg05294243 | chr19: 51569106 | KLK13.TSS1500 | SCLC-A | F | chr19: 51568154- | S_Shore |
| | | | | | 51568412 | |
| cg05581469 | chr12: 49413435 | MLL2.UTR3 | SCLC-A | F | chr12: 49412299- | S_Shore |
| | | | | | 49412756 | |
| cg05639747 | chr12: 49740942 | DNAJC22.TSS200 | SCLC-A | R | chr12: 49738680- | S_Shore |
| | | | | | 49740841 | |
| cg13296983 | chr12: 6436553 | PLEKHG6.GENEBODY | SCLC-A | F | chr12: 6438272-6438931 | N_Shore |
| cg25989783 | chr8: 102699486 | NCALD.UTR3 | SCLC-A | F | | |
| cg01448470 | chr1: 18518226 | IGSF21.GENEBODY | SCLC-A | R | | |
| cg06747907 | chr1: 16091100 | FBLIM1.UTR5 | SCLC-A | R | | |
| cg01864032 | chrX: 69674332 | DLG3.GENEBODY | SCLC-A | F | chrX: 69674799- | N_Shore |
| | | | | | 69675305 | |
| cg04138378 | chr7: 95849192 | MIR591.TSS200 | SCLC-A | R | | |
| cg12503394 | chr7: 1596118 | TMEM184A.TSS200 | SCLC-A | R | | |
| cg15152301 | chr3: 50425602 | CACNA2D2.GENEBODY | SCLC-A | F | | |
| cg15787636 | chr17: 80866113 | TBCD.GENEBODY | SCLC-A | R | chr17: 80863327- | S_Shelf |
| | | | | | 80863743 | |
| cg16884042 | chr2: 223289683 | SGPP2.GENEBODY | SCLC-A | F | chr2: 223288922- | Island |
| | | | | | 223290013 | |
| cg18535456 | chr12: 112203801 | ALDH2.TSS1500 | SCLC-A | R | chr12: 112204498- | N_Shore |
| | | | | | 112204979 | |
| cg25882455 | chr22: 38599073 | MAFF.EXON1 | SCLC-A | F | chr22: 38597785- | S_Shore |
| | | | | | 38598988 | |
| cg26785346 | chr4: 186732936 | SORBS2.UTR5 | SCLC-A | R | | |
| cg01708552 | chr7: 100815485 | C7orf52.GENEBODY | SCLC-A | R | chr7: 100815484- | Island |
| | | | | | 100816995 | |
| cg02487823 | chr3: 197440451 | KIAA0226.GENEBODY | SCLC-A | F | | |
| cg02862897 | chr17: 79793088 | DYSFIP1.TSS200 | SCLC-A | F | chr17: 79790530- | S_Shore |
| | | | | | 79791553 | |
| cg05036991 | chr2: 155089854 | GALNT13.GENEBODY | SCLC-A | F | | |
| cg13036352 | chr6: 30651825 | KIAA1949.GENEBODY | SCLC-A | F | chr6: 30654392- | N_Shelf |
| | | | | | 30654934 | |
| cg13478289 | chr6: 33132650 | COL11A2.GENEBODY | SCLC-A | F | chr6: 33129291- | S_Shelf |
| | | | | | 33129718 | |
| cg13508369 | chr9: 129884026 | ANGPTL2.UTR5 | SCLC-A | R | | |
| cg14703553 | chr17: 70663493 | SLC39A11.GENEBODY | SCLC-A | R | | |
| cg15076368 | chr3: 39224943 | XIRP1.UTR3 | SCLC-A | R | chr3: 39222280- | S_Shelf |
| | | | | | 39222670 | |
| cg15204874 | chr1: 54822298 | SSBP3.GENEBODY | SCLC-A | R | chr1: 54821990- | Island |
| | | | | | 54822333 | |
| cg18010718 | chr17: 74075195 | ZACN.TSS200 | SCLC-A | R | chr17: 74070404- | S_Shore |
| | | | | | 74073530 | |
| cg18211633 | chr12: 122205609 | TMEM120B.GENEBODY | SCLC-A | R | | |
| cg19095827 | chr3: 107243066 | BBX.UTR5 | SCLC-A | F | chr3: 107241845- | S_Shore |
| | | | | | 107242731 | |
| cg19287817 | chr19: 15391339 | BRD4.TSS200 | SCLC-A | F | | |
| cg19589800 | chr12: 93820263 | UBE2N.GENEBODY | SCLC-A | F | | |
| cg23350812 | chr2: 236415734 | AGAP1.GENEBODY | SCLC-A | R | | |
| cg00985958 | chr6: 71605996 | B3GAT2.GENEBODY | SCLC-A | F | | |
| cg01033299 | chr12: 53601015 | ITGB7.TSS200 | SCLC-A | R | | |
| cg02492946 | chr1: 21667630 | ECE1.GENEBODY | SCLC-A | R | chr1: 21671360- | N_Shelf |
| | | | | | 21672319 | |
| cg04617549 | chr19: 51569622 | KLK13.TSS1500 | SCLC-A | F | chr19: 51568154- | S_Shore |
| | | | | | 51568412 | |
| cg04918002 | chr16: 87423124 | FBXO31.GENEBODY | SCLC-A | R | chr16: 87425137- | N_Shelf |
| | | | | | 87426677 | |
| cg05487134 | chr17: 40489569 | STAT3.GENEBODY | SCLC-A | F | | |
| cg05928980 | chr1: 29567425 | PTPRU.GENEBODY | SCLC-A | R | chr1: 29562745- | S_Shelf |
| | | | | | 29565307 | |
| cg07824564 | chr1: 179050911 | TOR3A.TSS200 | SCLC-A | F | chr1: 179051075- | N_Shore |
| | | | | | 179051948 | |
| cg13261623 | chr17: 74462685 | AANAT.GENEBODY | SCLC-A | R | | |
| cg13698883 | chr9: 38422614 | IGFBPL1.GENEBODY | SCLC-A | F | chr9: 38423947- | N_Shore |
| | | | | | 38424584 | |
| cg26279814 | chr17: 37617607 | CDK12.TSS200 | SCLC-A | F | chr17: 37617531- | Island |
| | | | | | 37618045 | |
| cg26575738 | chr17: 44896168 | WNT3.TSS200 | SCLC-A | F | chr17: 44895800- | Island |
| | | | | | 44897025 | |
| cg00193613 | chr17: 77042810 | C1QTNF1.GENEBODY | SCLC-A | R | chr17: 77043741- | N_Shore |
| | | | | | 77044489 | |
| cg00715124 | chr6: 3227026 | TUBB2B.GENEBODY | SCLC-A | R | chr6: 3227025-3229688 | Island |
| cg03182958 | chr2: 223288923 | SGPP2.TSS1500 | SCLC-A | R | chr2: 223288922- | Island |
| | | | | | 223290013 | |
| cg05650846 | chr16: 3078142 | CCDC64B.GENEBODY | SCLC-A | R | chr16: 3078141-3078553 | Island |
| cg06201861 | chr16: 84877964 | CRISPLD2.GENEBODY | SCLC-A | F | | |
| cg07985864 | chr1: 10757892 | CASZ1.UTR5 | SCLC-A | R | chr1: 10757891- | Island |
| | | | | | 10758252 | |
| cg10544251 | chr1: 156215727 | PAQR6.UTR5 | SCLC-A | R | chr1: 156215368- | Island |
| | | | | | 156215899 | |
| cg23261073 | chr1: 9880771 | CLSTN1.GENEBODY | SCLC-A | R | chr1: 9883011-9884667 | N_Shelf |
| cg23551605 | chr2: 236442942 | AGAP1.GENEBODY | SCLC-A | F | chr2: 236443815- | N_Shore |
| | | | | | 236444025 | |
| cg25482454 | chr2: 85988369 | ATOH8.GENEBODY | SCLC-A | R | | |
| cg03230858 | chr19: 751124 | C19orf21.TSS200 | SCLC-A | R | chr19: 746300-747161 | S_Shelf |
| cg03732014 | chr21: 30676184 | BACH1.UTR5 | SCLC-A | F | | |
| cg03880611 | chr5: 31889373 | PDZD2.GENEBODY | SCLC-A | F | | |
| cg06091094 | chr11: 44927766 | TSPAN18.UTR5 | SCLC-A | F | | |
| cg10426951 | chr1: 54821910 | SSBP3.GENEBODY | SCLC-A | F | chr1: 54821990- | N_Shore |
| | | | | | 54822333 | |
| cg16595667 | chr14: 77567533 | KIAA1737.UTR5 | SCLC-A | F | chr14: 77563965- | S_Shelf |
| | | | | | 77564697 | |
| cg17490445 | chr7: 32623673 | AVL9.UTR3 | SCLC-A | F | | |
| cg17699214 | chr4: 40890013 | APBB2.GENEBODY | SCLC-A | R | | |
| cg21094405 | chr13: 113656117 | MCF2L.GENEBODY | SCLC-A | R | chr13: 113656116- | Island |
| | | | | | 113656379 | |
| cg22807241 | chr7: 147709862 | MIR548F3.GENEBODY | SCLC-A | R | | |
| cg24837219 | chr3: 132772692 | TMEM108.UTR5 | SCLC-A | R | | |
| cg27630771 | chr1: 150533123 | ADAMTSL4.UTR3 | SCLC-A | R | chr1: 150534075- | N_Shore |
| | | | | | 150534301 | |
| cg00564723 | chr10: 75632066 | CAMK2G.GENEBODY | SCLC-A | F | chr10: 75633600- | N_Shore |
| | | | | | 75634796 | |
| cg01100330 | chr22: 39639518 | PDGFB.GENEBODY | SCLC-A | R | chr22: 39638219- | Island |
| | | | | | 39640966 | |
| cg04999036 | chr15: 42566375 | TMEM87A.TSS1500 | SCLC-A | R | chr15: 42566198- | Island |
| | | | | | 42566404 | |
| cg06130787 | chr19: 51523550 | KLK10.TSS1500 | SCLC-A | F | chr19: 51522004- | S_Shore |
| | | | | | 51522803 | |
| cg07610192 | chr2: 131130423 | PTPN18.GENEBODY | SCLC-A | R | chr2: 131129718- | Island |
| | | | | | 131130511 | |
| cg09923953 | chr19: 51525638 | KLK11.UTR3 | SCLC-A | F | chr19: 51522004- | S_Shelf |
| | | | | | 51522803 | |
| cg14414406 | chr9: 100326029 | TMOD1.GENEBODY | SCLC-A | F | | |
| cg19392379 | chr10: 123909426 | TACC2.GENEBODY | SCLC-A | R | | |
| cg19477361 | chr19: 2607903 | GNG7.UTR5 | SCLC-A | R | chr19: 2607725-2607967 | Island |
| cg22453183 | chr5: 89940953 | GPR98.GENEBODY | SCLC-A | F | | |
| cg09701233 | chr2: 236415814 | AGAP1.GENEBODY | SCLC-A | R | | |
| cg10585321 | chr9: 135995491 | RALGDS.GENEBODY | SCLC-A | R | chr9: 135995969- | N_Shore |
| | | | | | 135996954 | |
| cg12581298 | chr7: 1080836 | C7orf50.GENEBODY | SCLC-A | R | chr7: 1080835-1081069 | Island |
| cg13548543 | chr9: 34460044 | DNAI1.GENEBODY | SCLC-A | F | chr9: 34457375- | S_Shore |
| | | | | | 34458892 | |
| cg15055890 | chr3: 11747650 | VGLL4.UTR5 | SCLC-A | R | chr3: 11746577- | S_Shore |
| | | | | | 11746824 | |
| cg18322589 | chr10: 123909456 | TACC2.GENEBODY | SCLC-A | F | | |
| cg18814808 | chr5: 143698198 | KCTD16.GENEBODY | SCLC-A | R | | |
| cg19529471 | chr13: 35517634 | NBEA.GENEBODY | SCLC-A | R | | |
| cg25285041 | chr1: 41627631 | SCMH1.UTR5 | SCLC-A | R | | |
| cg00664743 | chr22: 31300253 | OSBP2.GENEBODY | SCLC-A | R | chr22: 31301702- | N_Shore |
| | | | | | 31302133 | |
| cg03717830 | chr6: 30182244 | TRIM26.TSS1500 | SCLC-A | R | chr6: 30180933- | S_Shore |
| | | | | | 30182187 | |
| cg15766101 | chr19: 46519228 | CCDC61.GENEBODY | SCLC-A | R | chr19: 46518283- | Island |
| | | | | | 46520080 | |
| cg17499826 | chr19: 46284986 | DMPK.GENEBODY | SCLC-A | F | chr19: 46282544- | S_Shore |
| | | | | | 46283127 | |
| cg18263988 | chr3: 111582491 | PHLDB2.UTR5 | SCLC-A | R | chr3: 111578237- | S_Shelf |
| | | | | | 111578739 | |
| cg21146273 | chr5: 152869193 | GRIA1.TSS1500 | SCLC-A | F | | |
| cg22540067 | chr2: 125305001 | CNTNAP5.GENEBODY | SCLC-A | F | | |
| cg02312706 | chr16: 87384599 | FBXO31.GENEBODY | SCLC-A | F | | |
| cg02445972 | chr17: 7166593 | CLDN7.TSS1500 | SCLC-A | R | chr17: 7164285-7166245 | S_Shore |
| cg03762081 | chr19: 51523565 | KLK10.TSS1500 | SCLC-A | F | chr19: 51522004- | S_Shore |
| | | | | | 51522803 | |
| cg14677615 | chr17: 27315628 | SEZ6.GENEBODY | SCLC-A | R | chr17: 27312969- | S_Shelf |
| | | | | | 27313283 | |
| cg14946113 | chr22: 44220872 | SULT4A1.UTR3 | SCLC-A | F | | |
| cg17585356 | chr17: 78857121 | RPTOR.GENEBODY | SCLC-A | R | chr17: 78857630- | N_Shore |
| | | | | | 78857866 | |
| cg19727124 | chr1: 6553227 | PLEKHG5.GENEBODY | SCLC-A | R | chr1: 6550083-6551115 | S_Shelf |
| cg23045716 | chr4: 1201881 | LOC100130872.GENEBODY | SCLC-A | R | chr4: 1202956-1203200 | N_Shore |
| cg23098020 | chr1: 153651886 | NPR1.EXON1 | SCLC-A | F | chr1: 153650830- | Island |
| | | | | | 153652375 | |
| cg24302354 | chr19: 48219029 | EHD2.UTR5 | SCLC-A | R | chr19: 48219831- | N_Shore |
| | | | | | 48220298 | |
| cg26530485 | chr1: 23496304 | LUZP1.TSS1500 | SCLC-A | F | | |
| cg27119919 | chr11: 60684332 | TMEM109.UTR5 | SCLC-A | F | chr11: 60681552- | S_Shelf |
| | | | | | 60682268 | |
| cg11634198 | chr17: 37764158 | NEUROD2.UTR5 | SCLC-A | F | chr17: 37764092- | Island |
| | | | | | 37764304 | |
| cg15798221 | chr17: 62773704 | LOC146880.GENEBODY | SCLC-A | R | chr17: 62774366- | N_Shore |
| | | | | | 62777797 | |
| cg17930201 | chr1: 155828174 | SYT11.TSS1500 | SCLC-A | R | chr1: 155829109- | N_Shore |
| | | | | | 155829431 | |
| cg19469357 | chr7: 8474830 | NXPH1.UTR5 | SCLC-A | R | chr7: 8473139-8475199 | Island |
| cg24112733 | chr7: 38255578 | STARD3NL.GENEBODY | SCLC-A | F | | |
| cg08897952 | chr11: 126229062 | ST3GAL4.UTR5 | SCLC-A | R | chr11: 126225355- | S_Shelf |
| | | | | | 126226073 | |
| cg00144550 | chr2: 100937796 | LONRF2.GENEBODY | SCLC-A | R | chr2: 100937779- | Island |
| | | | | | 100939059 | |
| cg06286135 | chr7: 157511479 | PTPRN2.GENEBODY | SCLC-A | F | | |
| cg11303127 | chr12: 49740807 | DNAJC22.TSS1500 | SCLC-A | F | chr12: 49738680- | Island |
| | | | | | 49740841 | |
| cg13969327 | chr7: 127526253 | SND1.GENEBODY | SCLC-A | F | | |
| cg18253971 | chr18: 21900171 | OSBPL1A.GENEBODY | SCLC-A | F | | |
| cg20104456 | chr19: 51568523 | KLK13.TSS200 | SCLC-A | R | chr19: 51568154- | S_Shore |
| | | | | | 51568412 | |
| cg24247667 | chr17: 74463439 | AANAT.TSS200 | SCLC-A | F | | |
| cg24901995 | chr11: 888570 | CHID1.GENEBODY | SCLC-A | R | chr11: 888684-888981 | N_Shore |
| cg27320207 | chr19: 15307057 | NOTCH3.GENEBODY | SCLC-A | R | chr19: 15306243- | Island |
| | | | | | 15307111 | |
| cg03042971 | chr17: 47301348 | PHOSPHO1.UTR3 | SCLC-A | R | chr17: 47301545- | N_Shore |
| | | | | | 47302477 | |
| cg03442858 | chr1: 14924535 | KIAA1026.TSS1500 | SCLC-A | F | chr1: 14924610- | N_Shore |
| | | | | | 14925993 | |
| cg05020685 | chr4: 40858965 | APBB2.GENEBODY | SCLC-A | F | chr4: 40858884- | Island |
| | | | | | 40859162 | |
| cg07035875 | chr16: 52557205 | TOX3.UTR5 | SCLC-A | R | | |
| cg07686441 | chr15: 52570517 | MIR1266.TSS1500 | SCLC-A | R | | |
| cg12105190 | chr6: 30653407 | KIAA1949.GENEBODY | SCLC-A | R | chr6: 30654392- | N_Shore |
| | | | | | 30654934 | |
| cg18079653 | chr10: 72648646 | PCBD1.TSS200 | SCLC-A | R | chr10: 72647738- | S_Shore |
| | | | | | 72648317 | |
| cg18090068 | chr3: 55693316 | C3orf51.GENEBODY | SCLC-A | R | | |
| cg14225450 | chr6: 169637832 | THBS2.GENEBODY | SCLC-A | F | chr6: 169641803- | N_Shelf |
| | | | | | 169642020 | |
| cg01200905 | chr16: 52480264 | TOX3.GENEBODY | SCLC-A | R | | |
| cg01119831 | chr7: 2119608 | MAD1L1.GENEBODY | SCLC-A | R | chr7: 2116367-2116781 | S_Shelf |
| cg01593787 | chr13: 111182949 | RAB20.GENEBODY | SCLC-A | F | | |
| cg07543967 | chr19: 49055443 | SULT2B1.EXON1 | SCLC-A | R | | |
| cg08389577 | chr6: 3229379 | TUBB2B.TSS1500 | SCLC-A | R | chr6: 3227025-3229688 | Island |
| cg13796218 | chr1: 11750663 | MAD2L2.UTR5 | SCLC-A | R | chr1: 11750701- | N_Shore |
| | | | | | 11752651 | |
| cg14278148 | chr9: 114663626 | UGCG.GENEBODY | SCLC-A | F | chr9: 114658618- | S_Shelf |
| | | | | | 114659773 | |
| cg15927865 | chr10: 104159579 | NFKB2.GENEBODY | SCLC-A | F | chr10: 104159096- | S_Shore |
| | | | | | 104159536 | |
| cg16451995 | chr16: 8942340 | PMM2.UTR3 | SCLC-A | R | | |
| cg17523945 | chr19: 46732351 | IGFL1.TSS1500 | SCLC-A | F | | |
| cg17677988 | chr4: 10687769 | CLNK.TSS1500 | SCLC-A | F | | |
| cg21323630 | chr3: 51995784 | PCBP4.UTR5 | SCLC-A | R | | |
| cg23712594 | chr1: 226591890 | PARP1.GENEBODY | SCLC-N | R | chr1: 226595333- | N_Shelf |
| | | | | | 226595939 | |
| cg17170262 | chr1: 184715203 | EDEM3.GENEBODY | SCLC-N | F | | |
| cg05338397 | chr1: 15466556 | C1orf126.GENEBODY | SCLC-N | R | | |
| cg23682934 | chr17: 42885322 | GJC1.UTR5 | SCLC-N | R | | |
| cg06195280 | chr17: 6658706 | XAF1.TSS1500 | SCLC-N | F | | |
| cg01766396 | chr2: 198436483 | RFTN2.UTR3 | SCLC-N | F | | |
| cg07990556 | chr4: 159792176 | FNIP2.GENEBODY | SCLC-N | F | | |
| cg24899626 | chr8: 10512724 | RP1L1.TSS200 | SCLC-N | F | | |
| cg19028997 | chr1: 205603040 | ELK4.TSS1500 | SCLC-N | R | chr1: 205600493- | S_Shore |
| | | | | | 205601432 | |
| cg12562403 | chr12: 3214145 | TSPAN9.UTR5 | SCLC-N | R | | |
| cg08228249 | chr22: 22062300 | YPEL1.GENEBODY | SCLC-N | F | chr22: 22058079- | S_Shelf |
| | | | | | 22058304 | |
| cg27634115 | chr2: 42423477 | EML4.GENEBODY | SCLC-N | F | | |
| cg03861347 | chr20: 46412713 | SULF2.UTR5 | SCLC-N | F | chr20: 46413742- | N_Shore |
| | | | | | 46415507 | |
| cg07941953 | chr20: 10508668 | C20orf94.GENEBODY | SCLC-N | R | | |
| cg03187411 | chr7: 110482491 | IMMP2L.GENEBODY | SCLC-N | R | | |
| cg03223734 | chr20: 20180211 | C20orf26.GENEBODY | SCLC-N | R | | |
| cg23793194 | chr17: 37763735 | NEUROD2.UTR5 | SCLC-N | R | chr17: 37764092- | N_Shore |
| | | | | | 37764304 | |
| cg07831164 | chr16: 10836225 | NUBP1.TSS1500 | SCLC-N | R | chr16: 10837584- | N_Shore |
| | | | | | 10838196 | |
| cg20674206 | chr1: 63004738 | DOCK7.GENEBODY | SCLC-N | F | | |
| cg22721827 | chr16: 2520958 | NTN3.TSS1500 | SCLC-N | F | chr16: 2521086-2525929 | N_Shore |
| cg27096416 | chr7: 140599947 | BRAF.GENEBODY | SCLC-N | F | | |
| cg03419151 | chr17: 6658728 | XAF1.TSS1500 | SCLC-N | F | | |
| cg05420057 | chr17: 36721297 | SRCIN1.GENEBODY | SCLC-N | F | chr17: 36719544- | S_Shore |
| | | | | | 36719938 | |
| cg05950943 | chr16: 2520971 | NTN3.TSS1500 | SCLC-N | F | chr16: 2521086-2525929 | N_Shore |
| cg03243893 | chr8: 2005683 | MYOM2.GENEBODY | SCLC-N | R | chr8: 2003809-2004059 | S_Shore |
| cg20255723 | chr22: 45073031 | PRR5.UTR5 | SCLC-N | R | | |
| cg25275372 | chr17: 964596 | ABR.GENEBODY | SCLC-N | F | | |
| cg00284708 | chr8: 43147581 | POTEA.TSS200 | SCLC-N | F | | |
| cg09305096 | chr2: 189191655 | GULP1.UTR5 | SCLC-N | R | | |
| cg16696035 | chr17: 17126176 | FLCN.GENEBODY | SCLC-N | R | chr17: 17124698- | S_Shore |
| | | | | | 17124931 | |
| cg02966899 | chr15: 64793532 | ZNF609.GENEBODY | SCLC-N | R | | |
| cg09606564 | chr17: 19290353 | MFAP4.GENEBODY | SCLC-N | F | | |
| cg00332588 | chr8: 43147564 | POTEA.TSS200 | SCLC-N | R | | |
| cg08041350 | chr17: 25819112 | KSR1.UTR5 | SCLC-N | R | | |
| cg14074652 | chr9: 136662289 | VAV2.GENEBODY | SCLC-N | F | chr9: 136659111- | S_Shelf |
| | | | | | 136659329 | |
| cg19963589 | chr1: 54822103 | SSBP3.GENEBODY | SCLC-N | F | chr1: 54821990- | Island |
| | | | | | 54822333 | |
| cg09309024 | chr16: 29837267 | MVP.UTR5 | SCLC-N | F | | |
| cg11952344 | chr17: 80398854 | HEXDC.GENEBODY | SCLC-N | F | chr17: 80397431- | S_Shore |
| | | | | | 80397642 | |
| cg20431135 | chr17: 19290762 | MFAP4.TSS1500 | SCLC-N | F | | |
| cg19155699 | chr3: 196050902 | TM4SF19.GENEBODY | SCLC-N | F | | |
| cg23704362 | chr8: 67405742 | C8orf46.EXON1 | SCLC-N | F | | |
| cg19991383 | chr3: 55034387 | CACNA2D3.GENEBODY | SCLC-N | F | | |
| cg00172603 | chr1: 54822031 | SSBP3.GENEBODY | SCLC-N | F | chr1: 54821990- | Island |
| | | | | | 54822333 | |
| cg06431681 | chr2: 202330990 | STRADB.GENEBODY | SCLC-N | F | | |
| cg14461974 | chr2: 3318039 | TSSC1.GENEBODY | SCLC-N | R | chr2: 3317676-3317935 | S_Shore |
| cg15119221 | chr17: 19290755 | MFAP4.TSS1500 | SCLC-N | F | | |
| cg21831649 | chr1: 16050377 | PLEKHM2.GENEBODY | SCLC-N | R | chr1: 16053616- | N_Shelf |
| | | | | | 16053975 | |
| cg06087554 | chr16: 31151917 | PRSS36.GENEBODY | SCLC-N | R | chr16: 31153556- | N_Shore |
| | | | | | 31154474 | |
| cg01293647 | chr17: 37764264 | NEUROD2.TSS200 | SCLC-N | R | chr17: 37764092- | Island |
| | | | | | 37764304 | |
| cg14281144 | chr9: 38421899 | IGFBPL1.GENEBODY | SCLC-N | F | chr9: 38423947- | N_Shelf |
| | | | | | 38424584 | |
| cg13726218 | chr13: 72438249 | DACH1.GENEBODY | SCLC-N | R | chr13: 72439362- | N_Shore |
| | | | | | 72440059 | |
| cg17892159 | chr2: 42274610 | PKDCC.TSS1500 | SCLC-N | F | | |
| cg10414350 | chr8: 10210282 | MSRA.GENEBODY | SCLC-N | R | | |
| cg20326410 | chr1: 53600821 | SLC1A7.GENEBODY | SCLC-N | R | | |
| cg06785060 | chr1: 42180981 | HIVEP3.UTR5 | SCLC-N | F | | |
| cg07685557 | chr16: 85685994 | KIAA0182.GENEBODY | SCLC-N | F | chr16: 85684107- | S_Shore |
| | | | | | 85684491 | |
| cg08520443 | chr17: 746641 | NXN.GENEBODY | SCLC-N | R | chr17: 744617-744907 | S_Shore |
| cg17322655 | chr2: 242802127 | PDCD1.TSS1500 | SCLC-N | F | chr2: 242805752- | N_Shelf |
| | | | | | 242806034 | |
| cg00545232 | chr13: 93923232 | GPC6.GENEBODY | SCLC-N | R | | |
| cg11019127 | chr11: 115369647 | CADM1.GENEBODY | SCLC-N | F | chr11: 115373574- | N_Shelf |
| | | | | | 115375881 | |
| cg08151442 | chr2: 238632428 | LRRFIP1.GENEBODY | SCLC-N | F | | |
| cg16719771 | chr8: 43147554 | POTEA.TSS200 | SCLC-N | R | | |
| cg20944379 | chr1: 207069392 | IL24.TSS1500 | SCLC-N | R | | |
| cg03679521 | chr17: 37764153 | NEUROD2.UTR5 | SCLC-N | F | chr17: 37764092- | Island |
| | | | | | 37764304 | |
| cg09440603 | chr21: 43105616 | NCRNA00111.GENEBODY | SCLC-N | F | chr21: 43109349- | N_Shelf |
| | | | | | 43109687 | |
| cg07360805 | chr20: 36148457 | BLCAP.UTR5 | SCLC-N | F | chr20: 36148603- | N_Shore |
| | | | | | 36150136 | |
| cg01796800 | chr17: 55595732 | MSI2.GENEBODY | SCLC-N | F | | |
| cg06429887 | chr14: 24600500 | FITM1.TSS200 | SCLC-N | F | chr14: 24601498- | N_Shore |
| | | | | | 24601700 | |
| cg10603757 | chr22: 37621745 | RAC2.UTR3 | SCLC-N | R | | |
| cg13440795 | chr8: 10210267 | MSRA.GENEBODY | SCLC-N | R | | |
| cg03889044 | chr2: 242802099 | PDCD1.TSS1500 | SCLC-N | F | chr2: 242805752- | N_Shelf |
| | | | | | 242806034 | |
| cg08200851 | chr6: 5472095 | FARS2.GENEBODY | SCLC-N | F | | |
| cg09414280 | chr12: 120130253 | CIT.GENEBODY | SCLC-N | R | chr12: 120127973- | S_Shore |
| | | | | | 120128291 | |
| cg21641186 | chr3: 126200897 | UROC1.UTR3 | SCLC-N | R | | |
| cg21830368 | chr17: 75373327 | SEPT9.UTR5 | SCLC-N | R | chr17: 75368688- | S_Shelf |
| | | | | | 75370506 | |
| cg04951371 | chr2: 3317860 | TSSC1.GENEBODY | SCLC-N | F | chr2: 3317676-3317935 | Island |
| cg21557724 | chr2: 3317928 | TSSC1.GENEBODY | SCLC-N | R | chr2: 3317676-3317935 | Island |
| cg12716832 | chr22: 45073165 | PRR5.UTR5 | SCLC-N | R | | |
| cg04436083 | chr4: 24567220 | DHX15.GENEBODY | SCLC-N | R | | |
| cg11125249 | chr3: 148737622 | GYG1.GENEBODY | SCLC-N | F | | |
| cg11993925 | chr19: 44307056 | LYPD5.TSS1500 | SCLC-N | F | chr19: 44302665- | S_Shelf |
| | | | | | 44303176 | |
| cg15293827 | chr4: 2827194 | SH3BP2.GENEBODY | SCLC-N | F | | |
| cg27531366 | chr1: 27627058 | WDTC1.GENEBODY | SCLC-N | F | | |
| cg01943029 | chr16: 31715088 | C16orf67.GENEBODY | SCLC-N | F | chr16: 31711839- | S_Shelf |
| | | | | | 31712343 | |
| cg13027937 | chr7: 152525220 | ACTR3B.GENEBODY | SCLC-N | F | | |
| cg16623710 | chr22: 45073049 | PRR5.UTR5 | SCLC-N | F | | |
| cg00679520 | chr12: 63133578 | PPM1H.GENEBODY | SCLC-N | F | | |
| cg03742724 | chr6: 161838997 | PARK2.GENEBODY | SCLC-N | R | | |
| cg14300048 | chr9: 72046240 | APBA1.UTR3 | SCLC-N | F | | |
| cg09720012 | chr4: 108637177 | PAPSS1.GENEBODY | SCLC-N | R | chr4: 108640845- | N_Shelf |
| | | | | | 108641835 | |
| cg22689630 | chr19: 51568918 | KLK13.TSS1500 | SCLC-N | F | chr19: 51568154- | S_Shore |
| | | | | | 51568412 | |
| cg27068996 | chr19: 51513883 | KLK9.TSS1500 | SCLC-N | F | | |
| cg09535960 | chr8: 23193704 | LOXL2.GENEBODY | SCLC-N | F | | |
| cg12575928 | chr11: 22687660 | GAS2.TSS1500 | SCLC-N | R | chr11: 22688285- | N_Shore |
| | | | | | 22688617 | |
| cg02333237 | chr14: 50802971 | CDKL1.GENEBODY | SCLC-N | R | | |
| cg02734358 | chr4: 90227074 | GPRIN3.UTR5 | SCLC-N | R | chr4: 90228714- | N_Shore |
| | | | | | 90229010 | |
| cg03118626 | chr19: 51523252 | KLK10.UTR5 | SCLC-N | R | chr19: 51522004- | S_Shore |
| | | | | | 51522803 | |
| cg06358171 | chr1: 54822008 | SSBP3.GENEBODY | SCLC-N | R | chr1: 54821990- | Island |
| | | | | | 54822333 | |
| cg06440275 | chr8: 2005701 | MYOM2.GENEBODY | SCLC-N | R | chr8: 2003809-2004059 | S_Shore |
| cg09393675 | chr17: 37764250 | NEUROD2.TSS200 | SCLC-N | R | chr17: 37764092- | Island |
| | | | | | 37764304 | |
| cg20164964 | chr12: 3213850 | TSPAN9.UTR5 | SCLC-N | R | | |
| cg02175739 | chr11: 875980 | CHID1.GENEBODY | SCLC-N | R | | |
| cg08969377 | chr17: 80915263 | B3GNTL1.GENEBODY | SCLC-N | R | chr17: 80918218- | N_Shelf |
| | | | | | 80919258 | |
| cg00317626 | chr1: 53600741 | SLC1A7.GENEBODY | SCLC-N | F | | |
| cg09382492 | chr17: 74463681 | AANAT.GENEBODY | SCLC-N | F | | |
| cg02516456 | chr11: 1025091 | MUC6.GENEBODY | SCLC-N | R | chr11: 1024746-1025359 | Island |
| cg03737367 | chr19: 50005045 | MIR150.TSS1500 | SCLC-N | R | | |
| cg13446863 | chr9: 139259726 | CARD9.GENEBODY | SCLC-N | R | chr9: 139257842- | S_Shore |
| | | | | | 139259075 | |
| cg02048220 | chr14: 91141833 | TTC7B.GENEBODY | SCLC-N | R | | |
| cg06760830 | chr1: 47079672 | MOBKL2C.GENEBODY | SCLC-N | F | chr1: 47078580- | S_Shore |
| | | | | | 47078967 | |
| cg11912239 | chr17: 76220608 | BIRC5.UTR3 | SCLC-N | F | | |
| cg13525276 | chr14: 81426012 | TSHR.GENEBODY | SCLC-N | F | | |
| cg14025021 | chr15: 43809510 | MAP1A.TSS1500 | SCLC-N | F | | |
| cg16960943 | chr2: 236417185 | AGAP1.GENEBODY | SCLC-N | R | | |
| cg17741629 | chr13: 50024703 | SETDB2.TSS1500 | SCLC-N | R | | |
| cg04135110 | chr5: 346695 | AHRR.GENEBODY | SCLC-N | R | chr5: 343449-344535 | S_Shelf |
| cg18722086 | chr3: 113380072 | KIAA2018.GENEBODY | SCLC-N | R | | |
| cg23141629 | chr13: 45792582 | GTF2F2.GENEBODY | SCLC-N | R | | |
| cg23571857 | chr17: 6658898 | XAF1.TSS1500 | SCLC-N | F | | |
| cg05416911 | chr12: 7176049 | C1S.GENEBODY | SCLC-N | F | | |
| cg15341350 | chr12: 119446891 | SRRM4.GENEBODY | SCLC-N | F | | |
| cg15382095 | chr17: 37762367 | NEUROD2.GENEBODY | SCLC-N | F | chr17: 37761691- | Island |
| | | | | | 37763551 | |
| cg00795812 | chr2: 242802009 | PDCD1.TSS1500 | SCLC-N | F | chr2: 242805752- | N_Shelf |
| | | | | | 242806034 | |
| cg14931683 | chr17: 35872880 | DUSP14.GENEBODY | SCLC-N | R | chr17: 35872722- | Island |
| | | | | | 35873026 | |
| cg05467716 | chr17: 62774079 | LOC146880.GENEBODY | SCLC-N | F | chr17: 62774366- | N_Shore |
| | | | | | 62777797 | |
| cg12097330 | chr17: 79050221 | BAIAP2.GENEBODY | SCLC-N | R | chr17: 79051892- | N_Shore |
| | | | | | 79052534 | |
| cg12517843 | chr19: 17361126 | USHBP1.GENEBODY | SCLC-N | F | chr19: 17356979- | S_Shelf |
| | | | | | 17357500 | |
| cg25216196 | chr17: 19290520 | MFAP4.TSS200 | SCLC-N | R | | |
| cg27051683 | chr2: 242802069 | PDCD1.TSS1500 | SCLC-N | F | chr2: 242805752- | N_Shelf |
| | | | | | 242806034 | |
| cg07290310 | chr3: 125741734 | SLC41A3.GENEBODY | SCLC-N | R | | |
| cg16360668 | chr1: 19265059 | IFFO2.GENEBODY | SCLC-N | R | | |
| cg12237784 | chr7: 44187017 | GCK.GENEBODY | SCLC-N | F | chr7: 44184770- | S_Shore |
| | | | | | 44185695 | |
| cg00175150 | chr1: 150486261 | ECM1.UTR3 | SCLC-N | R | chr1: 150488036- | N_Shore |
| | | | | | 150488416 | |
| cg14292870 | chr12: 6887543 | LAG3.GENEBODY | SCLC-N | F | | |
| cg16007395 | chr1: 33231070 | KIAA1522.GENEBODY | SCLC-N | F | | |
| cg24980413 | chr5: 346987 | AHRR.GENEBODY | SCLC-N | R | chr5: 343449-344535 | S_Shelf |
| cg10827768 | chr2: 28387339 | BRE.GENEBODY | SCLC-N | R | | |
| cg20340501 | chr2: 233141525 | DIS3L2.GENEBODY | SCLC-N | F | | |
| cg23271234 | chr2: 71126275 | VAX2.TSS1500 | SCLC-N | F | chr2: 71126720- | N_Shore |
| | | | | | 71129209 | |
| cg03162164 | chr7: 790384 | HEATR2.GENEBODY | SCLC-N | F | chr7: 791984-792238 | N_Shore |
| cg07727884 | chr17: 19290503 | MFAP4.TSS200 | SCLC-N | R | | |
| cg27417749 | chr15: 69449518 | MIR548H4.GENEBODY | SCLC-N | R | chr15: 69452451- | N_Shelf |
| | | | | | 69453939 | |
| cg06603325 | chr17: 39887725 | HAP1.GENEBODY | SCLC-N | F | chr17: 39889956- | N_Shelf |
| | | | | | 39891489 | |
| cg03446876 | chr7: 129414697 | MIR96.TSS200 | SCLC-N | R | chr7: 129418286- | N_Shelf |
| | | | | | 129422509 | |
| cg03454775 | chr8: 2012595 | MYOM2.GENEBODY | SCLC-N | R | | |
| cg09427374 | chr2: 86095320 | ST3GAL5.TSS1500 | SCLC-N | R | | |
| cg10825315 | chr14: 81425912 | TSHR.GENEBODY | SCLC-N | R | | |
| cg17096191 | chr1: 162039224 | NOS1AP.TSS1500 | SCLC-N | F | chr1: 162039450- | N_Shore |
| | | | | | 162040052 | |
| cg19784401 | chr11: 46457992 | AMBRA1.GENEBODY | SCLC-N | F | | |
| cg04876127 | chr1: 33618450 | TRIM62.GENEBODY | SCLC-N | R | | |
| cg11312087 | chr13: 100944053 | PCCA.GENEBODY | SCLC-N | R | | |
| cg14682005 | chr6: 15337603 | JARID2.GENEBODY | SCLC-N | R | | |
| cg16198315 | chr13: 72438678 | DACH1.GENEBODY | SCLC-N | R | chr13: 72439362- | N_Shore |
| | | | | | 72440059 | |
| cg20721467 | chr10: 76597927 | MYST4.UTR5 | SCLC-N | F | | |
| cg23894058 | chr19: 51537828 | KLK12.GENEBODY | SCLC-N | F | chr19: 51535218- | S_Shelf |
| | | | | | 51535475 | |
| cg00892353 | chr8: 124247948 | C8orf76.GENEBODY | SCLC-N | R | | |
| cg08472243 | chr16: 427912 | TMEM8A.GENEBODY | SCLC-N | F | chr16: 426038-426290 | S_Shore |
| cg10135894 | chr4: 7862655 | AFAP1.GENEBODY | SCLC-N | F | | |
| cg01146614 | chr6: 159235211 | EZR.GENEBODY | SCLC-N | F | chr6: 159239081- | N_Shelf |
| | | | | | 159240775 | |
| cg03470159 | chr8: 10261798 | MSRA.GENEBODY | SCLC-N | R | chr8: 10261900- | N_Shore |
| | | | | | 10262222 | |
| cg04629204 | chr1: 26348325 | EXTL1.EXON1 | SCLC-N | F | | |
| cg15511928 | chr1: 201895256 | LMOD1.GENEBODY | SCLC-N | R | | |
| cg18482164 | chr17: 37764118 | NEUROD2.UTR5 | SCLC-N | R | chr17: 37764092- | Island |
| | | | | | 37764304 | |
| cg23732608 | chr20: 20180469 | C20orf26.GENEBODY | SCLC-N | F | | |
| cg24355809 | chr17: 746574 | NXN.GENEBODY | SCLC-N | R | chr17: 744617-744907 | S_Shore |
| cg19595760 | chr1: 25948511 | MAN1C1.GENEBODY | SCLC-N | F | chr1: 25944099- | S_Shelf |
| | | | | | 25944772 | |
| cg08853318 | chr17: 71089186 | SLC39A11.TSS1500 | SCLC-N | F | | |
| cg21706038 | chr2: 3317693 | TSSC1.GENEBODY | SCLC-N | R | chr2: 3317676-3317935 | Island |
| cg26577529 | chr12: 52866845 | KRT6C.GENEBODY | SCLC-N | R | | |
| cg01179256 | chr12: 105141965 | CHST11.GENEBODY | SCLC-N | F | | |
| cg02069944 | chr17: 79191523 | AZI1.GENEBODY | SCLC-N | R | | |
| cg13409077 | chr9: 35618528 | CD72.TSS200 | SCLC-N | R | chr9: 35616980- | S_Shore |
| | | | | | 35617324 | |
| cg13700197 | chr9: 98221975 | PTCH1.GENEBODY | SCLC-N | R | | |
| cg13867915 | chr1: 27050039 | ARID1A.GENEBODY | SCLC-N | F | | |
| cg24083274 | chr7: 121977191 | CADPS2.GENEBODY | SCLC-N | F | | |
| cg05337753 | chr17: 75373407 | SEPT9.UTR5 | SCLC-N | F | chr17: 75368688- | S_Shelf |
| | | | | | 75370506 | |
| cg07533819 | chr5: 71407465 | MAP1B.GENEBODY | SCLC-N | F | chr5: 71402877- | S_Shelf |
| | | | | | 71404868 | |
| cg17327404 | chr1: 33231109 | KIAA1522.GENEBODY | SCLC-N | R | | |
| cg02627403 | chr17: 73823769 | UNC13D.UTR3 | SCLC-N | R | | |
| cg11552585 | chr19: 3196206 | NCLN.GENEBODY | SCLC-N | F | chr19: 3196188-3196393 | Island |
| cg13406860 | chr9: 132881336 | GPR107.GENEBODY | SCLC-N | F | | |
| cg17492795 | chr1: 214637903 | PTPN14.GENEBODY | SCLC-N | R | | |
| cg18590709 | chr17: 49122713 | SPAG9.GENEBODY | SCLC-N | R | | |
| cg24054871 | chr22: 45073148 | PRR5.UTR5 | SCLC-N | R | | |
| cg13404038 | chr9: 126123344 | CRB2.GENEBODY | SCLC-N | F | | |
| cg16797751 | chr7: 105290218 | ATXN7L1.GENEBODY | SCLC-N | F | | |
| cg04607412 | chr22: 45073157 | PRR5.UTR5 | SCLC-N | R | | |
| cg11213150 | chr9: 129884457 | ANGPTL2.UTR5 | SCLC-N | F | | |
| cg19921389 | chr3: 128780164 | GP9.UTR5 | SCLC-N | R | | |
| cg10784542 | chr6: 169625369 | THBS2.GENEBODY | SCLC-N | R | chr6: 169622297- | S_Shelf |
| | | | | | 169622520 | |
| cg21303745 | chr17: 77959826 | TBC1D16.GENEBODY | SCLC-N | F | | |
| cg06786238 | chr1: 33231190 | KIAA1522.GENEBODY | SCLC-N | R | | |
| cg09021393 | chr7: 95849568 | MIR591.TSS1500 | SCLC-N | R | | |
| cg14121282 | chr9: 137268074 | RXRA.GENEBODY | SCLC-N | F | | |
| cg17697739 | chr6: 1873340 | GMDS.GENEBODY | SCLC-N | R | | |
| cg21885317 | chr8: 124248094 | C8orf76.GENEBODY | SCLC-N | F | | |
| cg01429256 | chr17: 17687834 | RAI1.UTR5 | SCLC-N | F | chr17: 17685016- | S_Shore |
| | | | | | 17687240 | |
| cg02582754 | chr6: 160327345 | MAS1.TSS1500 | SCLC-N | R | | |
| cg07997493 | chr17: 37761947 | NEUROD2.GENEBODY | SCLC-N | R | chr17: 37761691- | Island |
| | | | | | 37763551 | |
| cg21565781 | chr12: 123892621 | SETD8.UTR3 | SCLC-N | R | | |
| cg02381521 | chr21: 42775934 | MX2.GENEBODY | SCLC-N | F | | |
| cg09768257 | chr8: 11628346 | NEIL2.UTR5 | SCLC-N | R | chr8: 11627091- | S_Shore |
| | | | | | 11627668 | |
| cg12205591 | chr21: 44588552 | CRYAA.TSS1500 | SCLC-N | F | chr21: 44590612- | N_Shelf |
| | | | | | 44590826 | |
| cg18875674 | chr11: 73026651 | ARHGEF17.GENEBODY | SCLC-N | F | | |
| cg08958913 | chr19: 40193890 | LGALS14.TSS1500 | SCLC-N | R | | |
| cg20712415 | chr3: 159570903 | SCHIP1.GENEBODY | SCLC-N | F | | |
| cg24653772 | chr7: 788989 | HEATR2.GENEBODY | SCLC-N | F | chr7: 791984-792238 | N_Shelf |
| cg10529401 | chr1: 53587046 | SLC1A7.GENEBODY | SCLC-N | F | | |
| cg13909661 | chr12: 26386833 | SSPN.UTR3 | SCLC-N | R | | |
| cg14388093 | chr9: 133342842 | ASS1.GENEBODY | SCLC-N | F | | |
| cg27554156 | chr12: 13248725 | GSG1.GENEBODY | SCLC-N | F | | |
| cg00452755 | chr1: 28864993 | SNHG3- | SCLC-N | R | | |
| | | RCC1.GENEBODY | | | | |
| cg02807008 | chr19: 1910258 | ADAT3.UTR5 | SCLC-N | R | chr19: 1911923-1913406 | N_Shore |
| cg14448919 | chr5: 435040 | AHRR.UTR3 | SCLC-N | F | | |
| cg17873750 | chr1: 116382984 | NHLH2.UTR5 | SCLC-N | F | chr1: 116380359- | S_Shore |
| | | | | | 116382364 | |
| cg26844077 | chr16: 85658855 | KIAA0182.UTR5 | SCLC-N | F | chr16: 85658742- | Island |
| | | | | | 85658998 | |
| cg26902557 | chr11: 1585852 | DUSP8.GENEBODY | SCLC-N | F | | |
| cg06479434 | chr7: 128395929 | CALU.GENEBODY | SCLC-N | R | | |
| cg08811581 | chr11: 16902852 | PLEKHA7.GENEBODY | SCLC-N | F | | |
| cg08902205 | chr16: 85658997 | KIAA0182.UTR5 | SCLC-N | F | chr16: 85658742- | Island |
| | | | | | 85658998 | |
| cg18023842 | chr1: 160336475 | NHLH1.TSS1500 | SCLC-N | F | | |
| cg21448423 | chr1: 55013956 | ACOT11.EXON1 | SCLC-N | R | | |
| cg07923746 | chr1: 179050803 | TOR3A.TSS1500 | SCLC-N | F | chr1: 179051075- | N_Shore |
| | | | | | 179051948 | |
| cg10987071 | chr2: 217276744 | SMARCAL1.TSS1500 | SCLC-N | F | chr2: 217277037- | N_Shore |
| | | | | | 217277557 | |
| cg14423168 | chr8: 41400312 | GINS4.UTR3 | SCLC-N | R | | |
| cg07397891 | chr1: 246582071 | SMYD3.GENEBODY | SCLC-N | F | | |
| cg09997760 | chr3: 179169556 | GNB4.TSS200 | SCLC-N | R | chr3: 179168735- | Island |
| | | | | | 179169593 | |
| cg18405786 | chr19: 18746985 | KLHL26.TSS1500 | SCLC-N | F | | |
| cg24852561 | chr13: 100340080 | CLYBL.GENEBODY | SCLC-N | F | | |
| cg04702527 | chr4: 139940407 | CCRN4L.GENEBODY | SCLC-N | F | chr4: 139936443- | S_Shelf |
| | | | | | 139937428 | |
| cg23994112 | chr17: 75373457 | SEPT9.UTR5 | SCLC-N | R | chr17: 75368688- | S_Shelf |
| | | | | | 75370506 | |
| cg05428433 | chr16: 3614067 | NLRC3.GENEBODY | SCLC-N | F | chr16: 3614316-3614601 | N_Shore |
| cg09138688 | chr5: 49963801 | PARP8.GENEBODY | SCLC-N | F | chr5: 49962267- | S_Shore |
| | | | | | 49963777 | |
| cg11367404 | chr4: 170180146 | SH3RF1.GENEBODY | SCLC-N | R | | |
| cg15179499 | chr5: 362529 | AHRR.GENEBODY | SCLC-N | R | chr5: 356570-359526 | S_Shelf |
| cg20782117 | chr7: 95865631 | SLC25A13.GENEBODY | SCLC-N | R | | |
| cg23296791 | chr19: 40229766 | CLC.TSS1500 | SCLC-N | F | | |
| cg24524634 | chr7: 2800976 | GNA12.GENEBODY | SCLC-N | F | chr7: 2802440-2802698 | N_Shore |
| cg09309711 | chr19: 17322661 | MYO9B.UTR3 | SCLC-N | F | chr19: 17322471- | Island |
| | | | | | 17322988 | |
| cg18254848 | chr19: 40227949 | CLC.GENEBODY | SCLC-N | F | | |
| cg08157318 | chr12: 102224140 | GNPTAB.GENEBODY | SCLC-N | R | chr12: 102224262- | N_Shore |
| | | | | | 102224614 | |
| cg10833439 | chr21: 42793818 | MX1.UTR5 | SCLC-N | F | chr21: 42792293- | S_Shore |
| | | | | | 42792704 | |
| cg12102573 | chr17: 27924969 | ANKRD13B.GENEBODY | SCLC-N | F | chr17: 27919874- | S_Shelf |
| | | | | | 27921105 | |
| cg00756860 | chr1: 144852047 | PDE4DIP.UTR3 | SCLC-N | R | | |
| cg01120953 | chr5: 149643466 | CAMK2A.GENEBODY | SCLC-N | R | | |
| cg09980085 | chr19: 40174352 | LOC400696.UTR3 | SCLC-N | F | | |
| cg13913247 | chr4: 141207827 | SCOC.UTR5 | SCLC-N | F | | |
| cg17568996 | chr22: 42828125 | NFAM1.GENEBODY | SCLC-N | F | | |
| cg04571652 | chr3: 192959762 | MGC2889.GENEBODY | SCLC-N | F | chr3: 192958709- | S_Shore |
| | | | | | 192959373 | |
| cg06256596 | chr16: 66511944 | BEAN.GENEBODY | SCLC-N | R | | |
| cg06772157 | chr7: 100728711 | TRIM56.TSS200 | SCLC-N | F | chr7: 100732088- | N_Shelf |
| | | | | | 100732424 | |
| cg25368586 | chr13: 111561018 | ANKRD10.GENEBODY | SCLC-N | F | | |
| cg23202177 | chr1: 116383238 | NHLH2.UTR5 | SCLC-N | R | chr1: 116380359- | S_Shore |
| | | | | | 116382364 | |
| cg16419345 | chr17: 73976089 | ACOX1.TSS1500 | SCLC-N | R | chr17: 73974971- | S_Shore |
| | | | | | 73975731 | |
| cg22807608 | chr17: 17594719 | RAI1.UTR5 | SCLC-N | F | chr17: 17597824- | N_Shelf |
| | | | | | 17598398 | |
| cg26772172 | chr17: 77474395 | HRNBP3.UTR5 | SCLC-N | R | | |
| cg02566157 | chr11: 61025815 | VWCE.UTR3 | SCLC-N | R | | |
| cg02851203 | chr1: 54808689 | SSBP3.GENEBODY | SCLC-N | F | | |
| cg08247612 | chr1: 160336486 | NHLH1.TSS1500 | SCLC-N | F | | |
| cg20367923 | chr6: 169624501 | THBS2.GENEBODY | SCLC-N | R | chr6: 169622297- | S_Shore |
| | | | | | 169622520 | |
| cg22460466 | chr7: 1896154 | MAD1L1.GENEBODY | SCLC-N | F | chr7: 1896090-1896451 | Island |
| cg04530460 | chr6: 42858550 | C6orf226.UTR5 | SCLC-N | F | chr6: 42858169- | Island |
| | | | | | 42858960 | |
| cg07424295 | chr10: 92980030 | PCGF5.TSS1500 | SCLC-N | R | chr10: 92980347- | N_Shore |
| | | | | | 92980832 | |
| cg09135866 | chr19: 53238498 | ZNF611.TSS200 | SCLC-N | F | chr19: 53237861- | Island |
| | | | | | 53238499 | |
| cg22102078 | chr6: 130182512 | C6orf191.TSS200 | SCLC-N | R | | |
| cg04155280 | chr17: 26692719 | SEBOX.GENEBODY | SCLC-N | F | | |
| cg08976117 | chr3: 57064983 | ARHGEF3.GENEBODY | SCLC-N | R | | |
| cg02997295 | chr6: 169642301 | THBS2.GENEBODY | SCLC-N | F | chr6: 169641803- | S_Shore |
| | | | | | 169642020 | |
| cg08399444 | chr12: 13248548 | GSG1.GENEBODY | SCLC-N | F | | |
| cg27379887 | chr20: 1448498 | NSFL1C.TSS200 | SCLC-N | R | chr20: 1446936-1447729 | S_Shore |
| cg21831485 | chr7: 31594014 | CCDC129.GENEBODY | SCLC-N | F | | |
| cg20292636 | chr1: 204328377 | PLEKHA6.UTR5 | SCLC-N | F | | |
| cg23316807 | chr19: 56628171 | ZNF787.UTR5 | SCLC-N | F | chr19: 56631771- | N_Shelf |
| | | | | | 56633076 | |
| cg03361664 | chr11: 3187760 | OSBPL5.TSS1500 | SCLC-N | R | chr11: 3186131-3186768 | S_Shore |
| cg11759917 | chr17: 79655302 | HGS.GENEBODY | SCLC-N | R | chr17: 79655162- | Island |
| | | | | | 79655515 | |
| cg17861836 | chr17: 79202505 | C17orf56.UTR3 | SCLC-N | F | | |
| cg19046189 | chr19: 49316732 | HSD17B14.GENEBODY | SCLC-N | F | chr19: 49315545- | S_Shore |
| | | | | | 49315765 | |
| cg25342005 | chr1: 33648656 | TRIM62.TSS1500 | SCLC-N | R | chr1: 33646613- | S_Shore |
| | | | | | 33647878 | |
| cg08301181 | chr17: 73780373 | UNK.TSS1500 | SCLC-N | R | chr17: 73780846- | N_Shore |
| | | | | | 73781308 | |
| cg23490611 | chrX: 134475099 | ZNF75D.UTR5 | SCLC-N | R | chrX: 134477583- | N_Shelf |
| | | | | | 134477999 | |
| cg27569829 | chr18: 60984485 | BCL2.GENEBODY | SCLC-N | R | chr18: 60985503- | N_Shore |
| | | | | | 60985741 | |
| cg01157146 | chr21: 42877638 | TMPRSS2.GENEBODY | SCLC-N | R | chr21: 42878751- | N_Shore |
| | | | | | 42880674 | |
| cg03155027 | chr3: 49907692 | CAMKV.TSS1500 | SCLC-N | F | chr3: 49906737- | S_Shore |
| | | | | | 49907482 | |
| cg14269510 | chr13: 47204488 | LRCH1.GENEBODY | SCLC-N | R | | |
| cg18849256 | chr12: 32309670 | BICD1.GENEBODY | SCLC-N | F | | |
| cg21930140 | chr21: 42796537 | MX1.TSS1500 | SCLC-N | F | chr21: 42798146- | N_Shore |
| | | | | | 42798884 | |
| cg22377043 | chr15: 43809486 | MAP1A.TSS1500 | SCLC-N | F | | |
| cg00928231 | chr11: 67195771 | RPS6KB2.TSS200 | SCLC-N | F | chr11: 67195800- | N_Shore |
| | | | | | 67196190 | |
| cg04329478 | chr19: 40169839 | LOC400696.TSS200 | SCLC-N | F | | |
| cg05070839 | chr8: 10261641 | MSRA.GENEBODY | SCLC-N | F | chr8: 10261900- | N_Shore |
| | | | | | 10262222 | |
| cg16145834 | chr6: 33282321 | TAPBP.TSS200 | SCLC-N | R | chr6: 33282855- | N_Shore |
| | | | | | 33283196 | |
| cg01654446 | chr6: 33280476 | TAPBP.GENEBODY | SCLC-N | R | chr6: 33281112- | N_Shore |
| | | | | | 33281827 | |
| cg15396570 | chr1: 85931161 | DDAH1.TSS1500 | SCLC-N | F | chr1: 85929940- | Island |
| | | | | | 85931168 | |
| cg17637107 | chr8: 20054583 | ATP6V1B2.TSS200 | SCLC-N | R | chr8: 20054546- | Island |
| | | | | | 20055027 | |
| cg14373410 | chr14: 81426234 | TSHR.GENEBODY | SCLC-N | R | | |
| cg22692213 | chr12: 133533186 | ZNF605.TSS1500 | SCLC-N | F | chr12: 133532642- | S_Shore |
| | | | | | 133532979 | |
| cg01033600 | chr19: 7964703 | LRRC8E.GENEBODY | SCLC-N | F | chr19: 7964439-7964704 | Island |
| cg15472885 | chr3: 71056608 | FOXP1.GENEBODY | SCLC-N | R | | |
| cg23596678 | chr17: 79793000 | DYSFIP1.TSS200 | SCLC-N | R | chr17: 79790530- | S_Shore |
| | | | | | 79791553 | |
| cg16616765 | chr2: 197456534 | HECW2.UTR5 | SCLC-N | R | chr2: 197457392- | N_Shore |
| | | | | | 197458522 | |
| cg21221712 | chr15: 86132153 | AKAP13.GENEBODY | SCLC-N | F | | |
| cg26130023 | chr18: 45453609 | SMAD2.UTR5 | SCLC-N | R | chr18: 45456484- | N_Shelf |
| | | | | | 45458024 | |
| cg26539941 | chr2: 220306470 | SPEG.GENEBODY | SCLC-N | R | chr2: 220306855- | N_Shore |
| | | | | | 220307096 | |
| cg14962159 | chr13: 37441585 | SMAD9.GENEBODY | SCLC-N | F | | |
| cg03466032 | chr17: 39968802 | FKBP10.TSS200 | SCLC-N | R | chr17: 39967407- | S_Shore |
| | | | | | 39968604 | |
| cg22862003 | chr21: 42797588 | MX1.TSS1500 | SCLC-N | F | chr21: 42798146- | N_Shore |
| | | | | | 42798884 | |
| cg05801879 | chr16: 84101832 | MBTPS1.GENEBODY | SCLC-N | F | | |
| cg08093568 | chr7: 27626595 | HIBADH.GENEBODY | SCLC-N | F | | |
| cg14398557 | chr20: 1095840 | PSMF1.UTR5 | SCLC-N | R | chr20: 1099078-1099561 | N_Shelf |
| cg05152503 | chr20: 30407098 | MYLK2.TSS200 | SCLC-N | R | chr20: 30410547- | N_Shelf |
| | | | | | 30410832 | |
| cg15040499 | chr17: 35861263 | DUSP14.UTR5 | SCLC-N | R | | |
| cg04420932 | chr11: 67195621 | RPS6KB2.TSS1500 | SCLC-N | F | chr11: 67195800- | N_Shore |
| | | | | | 67196190 | |
| cg22970357 | chr2: 119600408 | EN1.UTR3 | SCLC-N | R | chr2: 119599458- | Island |
| | | | | | 119600966 | |
| cg10288437 | chr6: 158432894 | SYNJ2.GENEBODY | SCLC-N | F | | |
| cg20976787 | chr6: 43064460 | PTK7.GENEBODY | SCLC-N | R | | |
| cg10294732 | chr4: 41614397 | LIMCH1.GENEBODY | SCLC-N | F | | |
| cg19314992 | chr11: 76614479 | ACER3.GENEBODY | SCLC-N | R | | |
| cg00791283 | chr12: 79498966 | SYT1.UTR5 | SCLC-N | R | | |
| cg16125375 | chr5: 146298512 | PPP2R2B.UTR5 | SCLC-N | R | | |
| cg13663667 | chr19: 4180236 | SIRT6.GENEBODY | SCLC-N | F | chr19: 4182259-4182803 | N_Shelf |
| cg02711899 | chr6: 42410890 | TRERF1.UTR5 | SCLC-N | R | | |
| cg26181372 | chr9: 104198262 | ALDOB.TSS1500 | SCLC-N | R | | |
| cg22780475 | chr19: 45281526 | CBLC.EXON1 | SCLC-N | F | chr19: 45281133- | S_Shore |
| | | | | | 45281355 | |
| cg13127741 | chr20: 31331821 | COMMD7.TSS200 | SCLC-N | F | chr20: 31330957- | S_Shore |
| | | | | | 31331410 | |
| cg08352439 | chr7: 55637123 | VOPP1.GENEBODY | SCLC-N | F | chr7: 55639277- | N_Shelf |
| | | | | | 55640599 | |
| cg04528038 | chr16: 21171097 | TMEM159.UTR5 | SCLC-N | F | chr16: 21169810- | S_Shore |
| | | | | | 21170278 | |
| cg09336589 | chr17: 7107939 | DLG4.GENEBODY | SCLC-N | F | chr17: 7108304-7108654 | N_Shore |
| cg25976170 | chr22: 29707585 | GAS2L1.UTR3 | SCLC-N | F | chr22: 29707479- | Island |
| | | | | | 29707797 | |
| cg07557423 | chr4: 42657903 | ATP8A1.GENEBODY | SCLC-N | F | chr4: 42658278- | N_Shore |
| | | | | | 42659550 | |
| cg06342490 | chr2: 42795193 | MTA3.TSS1500 | SCLC-N | F | chr2: 42795847- | N_Shore |
| | | | | | 42796230 | |
| cg21574244 | chr15: 42568411 | GANC.GENEBODY | SCLC-N | F | chr15: 42565452- | S_Shelf |
| | | | | | 42565791 | |
| cg00551589 | chr16: 2626391 | PDPK1.GENEBODY | SCLC-N | R | chr16: 2627134-2627399 | N_Shore |
| cg07127888 | chr8: 41400789 | GINS4.UTR3 | SCLC-N | F | | |
| cg16293838 | chr1: 6330521 | ACOT7.GENEBODY | SCLC-N | F | | |
| cg17461336 | chr7: 99441559 | CYP3A43.GENEBODY | SCLC-N | R | | |
| cg26207423 | chr8: 28787952 | HMBOX1.UTR5 | SCLC-N | R | | |
| cg01489441 | chr16: 1764111 | MAPK8IP3.GENEBODY | SCLC-N | R | | |
| cg17397069 | chr5: 156110742 | SGCD.GENEBODY | SCLC-N | R | | |
| cg27434467 | chr10: 101686326 | NCRNA00093.TSS1500 | SCLC-N | F | | |
| cg15580167 | chr2: 175629219 | CHRNA1.TSS200 | SCLC-N | F | | |
| cg26245086 | chr4: 87860957 | AFF1.UTR5 | SCLC-N | F | chr4: 87855873- | S_Shelf |
| | | | | | 87857204 | |
| cg07696516 | chr4: 41614958 | LIMCH1.EXON1 | SCLC-N | F | | |
| cg16457351 | chr5: 176297315 | UNC5A.GENEBODY | SCLC-N | R | chr5: 176296541- | S_Shore |
| | | | | | 176296933 | |
| cg01372366 | chr11: 48041783 | PTPRJ.GENEBODY | SCLC-N | F | | |
| cg14414874 | chr9: 126157693 | DENND1A.GENEBODY | SCLC-N | F | chr9: 126154226- | S_Shelf |
| | | | | | 126154562 | |
| cg16039867 | chr1: 47023134 | MKNK1.GENEBODY | SCLC-N | R | | |
| cg23615892 | chr1: 215825505 | USH2A.GENEBODY | SCLC-N | R | | |
| cg13763232 | chr3: 14443428 | SLC6A6.TSS1500 | SCLC-N | R | chr3: 14443629- | N_Shore |
| | | | | | 14444577 | |
| cg00957901 | chr4: 41614842 | LIMCH1.TSS200 | SCLC-N | R | | |
| cg01794926 | chr15: 92470387 | SLCO3A1.GENEBODY | SCLC-N | R | | |
| cg03167954 | chr22: 29707782 | GAS2L1.UTR3 | SCLC-N | R | chr22: 29707479- | Island |
| | | | | | 29707797 | |
| cg22976732 | chr22: 28191914 | MN1.GENEBODY | SCLC-N | F | chr22: 28192793- | N_Shore |
| | | | | | 28198592 | |
| cg00927494 | chr11: 69924230 | ANO1.TSS200 | SCLC-N | R | chr11: 69924338- | N_Shore |
| | | | | | 69925197 | |
| cg21661983 | chr12: 72353490 | TPH2.GENEBODY | SCLC-N | F | | |
| cg00664609 | chr16: 2198075 | RAB26.TSS1500 | SCLC-N | R | chr16: 2198455-2199129 | N_Shore |
| cg16544220 | chr6: 33095030 | HLA-DPB2.GENEBODY | SCLC-N | F | | |
| cg00718444 | chr8: 145685564 | CYHR1.GENEBODY | SCLC-N | F | chr8: 145687389- | N_Shore |
| | | | | | 145687880 | |
| cg03227338 | chr11: 46998965 | C11orf49.GENEBODY | SCLC-N | F | | |
| cg13491737 | chr13: 111295728 | CARS2.GENEBODY | SCLC-N | R | chr13: 111294675- | S_Shore |
| | | | | | 111294900 | |
| cg23924526 | chr2: 45998548 | PRKCE.GENEBODY | SCLC-N | R | | |
| cg03180426 | chr9: 139964156 | C9orf140.GENEBODY | SCLC-N | R | chr9: 139964155- | Island |
| | | | | | 139965284 | |
| cg21579399 | chr2: 11890700 | LPIN1.UTR5 | SCLC-N | F | chr2: 11886249- | S_Shelf |
| | | | | | 11887207 | |
| cg00249974 | chr1: 156254149 | TMEM79.EXON1 | SCLC-N | R | | |
| cg15585294 | chr11: 22687641 | GAS2.TSS1500 | SCLC-N | R | chr11: 22688285- | N_Shore |
| | | | | | 22688617 | |
| cg10153942 | chr19: 54982500 | CDC42EP5.UTR5 | SCLC-N | R | chr19: 54982388- | Island |
| | | | | | 54982897 | |
| cg11282156 | chr3: 63428840 | SYNPR.UTR5 | SCLC-N | F | | |
| cg18003659 | chr2: 175629267 | CHRNA1.TSS200 | SCLC-N | R | | |
| cg22340762 | chr1: 183823538 | RGL1.GENEBODY | SCLC-N | F | | |
| cg00408605 | chr8: 141466079 | TRAPPC9.UTR5 | SCLC-P | R | chr8: 141467218- | N_Shore |
| | | | | | 141467927 | |
| cg00600617 | chr6: 107221887 | LOC553137.GENEBODY | SCLC-P | F | | |
| cg00618183 | chr17: 75143600 | SEC14L1.GENEBODY | SCLC-P | R | | |
| cg00625110 | chr16: 53741731 | FTO.GENEBODY | SCLC-P | R | chr16: 53737617- | S_Shelf |
| | | | | | 53738235 | |
| cg00703885 | chr2: 128440247 | LIMS2.TSS1500 | SCLC-P | F | | |
| cg00704369 | chr7: 81996559 | CACNA2D1.GENEBODY | SCLC-P | R | | |
| cg00706536 | chr17: 40609826 | ATP6VOA1.TSS1500 | SCLC-P | R | chr17: 40610746- | N_Shore |
| | | | | | 40611032 | |
| cg01149239 | chr3: 196746085 | MFI2.UTR3 | SCLC-P | F | | |
| cg01307483 | chr7: 129254604 | NRF1.UTR5 | SCLC-P | F | chr7: 129251237- | S_Shelf |
| | | | | | 129252441 | |
| cg01580044 | chr1: 114300656 | PHTF1.GENEBODY | SCLC-P | R | chr1: 114301514- | N_Shore |
| | | | | | 114302272 | |
| cg01626895 | chr12: 116425790 | MED13L.GENEBODY | SCLC-P | R | | |
| cg01653080 | chr14: 104205041 | PPP1R13B.GENEBODY | SCLC-P | F | chr14: 104205070- | N_Shore |
| | | | | | 104205329 | |
| cg01715088 | chr1: 6520772 | ESPN.UTR3 | SCLC-P | R | chr1: 6520584-6521955 | Island |
| cg02220284 | chr3: 71013485 | FOXP1.GENEBODY | SCLC-P | F | | |
| cg02253499 | chr3: 12602046 | MKRN2.GENEBODY | SCLC-P | F | chr3: 12598391- | S_Shelf |
| | | | | | 12599051 | |
| cg02330721 | chr16: 1764231 | MAPK8IP3.GENEBODY | SCLC-P | R | | |
| cg02519218 | chr12: 133424286 | CHFR.GENEBODY | SCLC-P | R | | |
| cg02793948 | chr11: 68095662 | LRP5.GENEBODY | SCLC-P | F | | |
| cg03075214 | chr11: 64867914 | C11orf2.GENEBODY | SCLC-P | R | chr11: 64863698- | S_Shelf |
| | | | | | 64863980 | |
| cg03147598 | chr2: 131104160 | IMP4.UTR3 | SCLC-P | F | chr2: 131099469- | S_Shelf |
| | | | | | 131100863 | |
| cg03264729 | chr11: 72347339 | PDE2A.GENEBODY | SCLC-P | R | | |
| cg03803789 | chr11: 111854003 | DIXDC1.GENEBODY | SCLC-P | R | | |
| cg04390667 | chr11: 46624867 | HARBI1.UTR3 | SCLC-P | R | | |
| cg05001389 | chr9: 135853034 | GFI1B.TSS1500 | SCLC-P | R | | |
| cg05661060 | chr1: 44074454 | PTPRF.GENEBODY | SCLC-P | F | chr1: 44070892- | S_Shelf |
| | | | | | 44071142 | |
| cg06206957 | chr7: 33080615 | NT5C3.TSS200 | SCLC-P | R | | |
| cg06604199 | chr20: 31331973 | COMMD7.TSS200 | SCLC-P | R | chr20: 31330957- | S_Shore |
| | | | | | 31331410 | |
| cg06669752 | chr5: 138534431 | SIL1.TSS1500 | SCLC-P | R | chr5: 138533870- | S_Shore |
| | | | | | 138534113 | |
| cg06832246 | chr11: 93224210 | C11orf75.UTR5 | SCLC-P | R | | |
| cg06839854 | chr2: 10536115 | HPCAL1.UTR5 | SCLC-P | R | | |
| cg07205154 | chr1: 176147105 | RFWD2.GENEBODY | SCLC-P | R | | |
| cg07313835 | chr1: 167342724 | POU2F1.GENEBODY | SCLC-P | F | | |
| cg07699978 | chr12: 63194001 | PPM1H.GENEBODY | SCLC-P | R | chr12: 63195657- | N_Shore |
| | | | | | 63195909 | |
| cg07859138 | chrX: 118751995 | SEPT6.UTR3 | SCLC-P | F | | |
| cg07956003 | chr7: 95433460 | DYNC111.UTR5 | SCLC-P | F | | |
| cg08101329 | chr17: 4884631 | CAMTA2.GENEBODY | SCLC-P | R | | |
| cg08238265 | chr14: 64682912 | SYNE2.EXON1 | SCLC-P | R | | |
| cg08781755 | chr2: 44172074 | LRPPRC.GENEBODY | SCLC-P | F | | |
| cg09169413 | chr12: 48103670 | P11.UTR3 | SCLC-P | F | chr12: 48099317- | S_Shelf |
| | | | | | 48099905 | |
| cg10386431 | chr1: 77879031 | AK5.GENEBODY | SCLC-P | F | | |
| cg10887937 | chr12: 112203795 | ALDH2.TSS1500 | SCLC-P | R | chr12: 112204498- | N_Shore |
| | | | | | 112204979 | |
| cg11019743 | chr6: 168267395 | MLLT4.GENEBODY | SCLC-P | F | | |
| cg11132582 | chr1: 167217689 | POU2F1.UTR5 | SCLC-P | F | | |
| cg11447040 | chr20: 37667339 | DHX35.UTR3 | SCLC-P | R | | |
| cg11701604 | chr11: 36095771 | LDLRAD3.GENEBODY | SCLC-P | F | | |
| cg13146674 | chr11: 68095623 | LRP5.GENEBODY | SCLC-P | R | | |
| cg13556548 | chr1: 226187876 | C1orf55.TSS1500 | SCLC-P | R | chr1: 226186803- | S_Shore |
| | | | | | 226187336 | |
| cg13578134 | chr15: 75081572 | CSK.UTR5 | SCLC-P | R | | |
| cg13619387 | chr9: 92050313 | SEMA4D.UTR5 | SCLC-P | R | | |
| cg13676816 | chr9: 91976972 | SEMA4D.UTR3 | SCLC-P | R | | |
| cg14211075 | chr9: 135818481 | TSC1.UTR5 | SCLC-P | R | chr9: 135819526- | N_Shore |
| | | | | | 135820529 | |
| cg14301030 | chr10: 264920 | ZMYND11.GENEBODY | SCLC-P | R | | |
| cg14302130 | chr6: 33032830 | HLA-DPA1.UTR3 | SCLC-P | R | | |
| cg14457284 | chr9: 135852984 | GFI1B.TSS1500 | SCLC-P | R | | |
| cg14701925 | chr3: 128980811 | COPG.GENEBODY | SCLC-P | F | | |
| cg14817765 | chr20: 2444494 | SNORD119.TSS1500 | SCLC-P | F | | |
| cg15337905 | chr7: 150807508 | AGAP3.GENEBODY | SCLC-P | R | | |
| cg15425670 | chr2: 42461109 | EML4.GENEBODY | SCLC-P | F | | |
| cg15724534 | chr2: 211018363 | C2orf67.GENEBODY | SCLC-P | R | | |
| cg15873449 | chr18: 12885569 | PTPN2.TSS1500 | SCLC-P | F | chr18: 12883285- | S_Shore |
| | | | | | 12884578 | |
| cg16318349 | chr1: 154917307 | PBXIP1.UTR3 | SCLC-P | R | | |
| cg16691863 | chr1: 28848173 | SNHG3- | SCLC-P | R | chr1: 28844653- | S_Shelf |
| | | RCC1.GENEBODY | | | 28845293 | |
| cg17308493 | chr10: 30634469 | MTPAP.GENEBODY | SCLC-P | F | chr10: 30637961- | N_Shelf |
| | | | | | 30638279 | |
| cg17355865 | chr11: 76887139 | MYO7A.GENEBODY | SCLC-P | R | chr11: 76890783- | N_Shelf |
| | | | | | 76890986 | |
| cg17758792 | chr21: 37620038 | DOPEY2.GENEBODY | SCLC-P | F | chr21: 37617467- | S_Shelf |
| | | | | | 37617872 | |
| cg18455249 | chr4: 56813898 | CEP135.TSS1500 | SCLC-P | R | chr4: 56814800- | N_Shore |
| | | | | | 56815511 | |
| cg18872426 | chr19: 38874037 | PSMD8.UTR3 | SCLC-P | F | chr19: 38876070- | N_Shelf |
| | | | | | 38876332 | |
| cg19125791 | chr2: 240265915 | HDAC4.GENEBODY | SCLC-P | F | | |
| cg19506380 | chr8: 98740612 | MTDH.UTR3 | SCLC-P | F | | |
| cg19918623 | chr7: 132661002 | CHCHD3.GENEBODY | SCLC-P | R | | |
| cg20059682 | chr12: 52201821 | SCN8A.UTR3 | SCLC-P | F | | |
| cg20467658 | chr15: 89933347 | LOC254559.GENEBODY | SCLC-P | R | | |
| cg20497631 | chr8: 8647440 | MFHAS1.GENEBODY | SCLC-P | R | | |
| cg20664313 | chr12: 133071297 | FBRSL1.GENEBODY | SCLC-P | R | chr12: 133073397- | N_Shelf |
| | | | | | 133073632 | |
| cg20716489 | chr3: 195591877 | TNK2.GENEBODY | SCLC-P | R | chr3: 195594836- | N_Shelf |
| | | | | | 195595494 | |
| cg21232015 | chr12: 133424221 | CHFR.GENEBODY | SCLC-P | R | | |
| cg21289124 | chr22: 26951249 | TPST2.UTR5 | SCLC-P | R | | |
| cg21535670 | chr8: 140798600 | TRAPPC9.GENEBODY | SCLC-P | F | | |
| cg21877565 | chr1: 156461550 | MEF2D.UTR5 | SCLC-P | F | | |
| cg21927971 | chr2: 17837235 | VSNL1.UTR3 | SCLC-P | R | | |
| cg21940081 | chr2: 131103235 | IMP4.GENEBODY | SCLC-P | R | chr2: 131099469- | S_Shelf |
| | | | | | 131100863 | |
| cg22542731 | chr2: 128440274 | LIMS2.TSS1500 | SCLC-P | F | | |
| cg23396732 | chr20: 31332170 | COMMD7.TSS1500 | SCLC-P | F | chr20: 31330957- | S_Shore |
| | | | | | 31331410 | |
| cg24136932 | chr8: 22281979 | SLC39A14.GENEBODY | SCLC-P | R | | |
| cg24440131 | chr1: 26880207 | MIR1976.TSS1500 | SCLC-P | R | | |
| cg24495350 | chr15: 72651198 | HEXA.GENEBODY | SCLC-P | F | | |
| cg24677222 | chr3: 140102647 | CLSTN2.GENEBODY | SCLC-P | F | | |
| cg25601286 | chr2: 169408033 | LASS6.GENEBODY | SCLC-P | R | | |
| cg25874150 | chr10: 34941561 | PARD3.GENEBODY | SCLC-P | R | | |
| cg26224018 | chr1: 64942153 | CACHD1.GENEBODY | SCLC-P | F | | |
| cg26542660 | chr4: 56813860 | CEP135.TSS1500 | SCLC-P | R | chr4: 56814800- | N_Shore |
| | | | | | 56815511 | |
| cg26579713 | chr15: 65701865 | IGDCC4.GENEBODY | SCLC-P | F | | |
| cg26797073 | chr2: 131129811 | PTPN18.GENEBODY | SCLC-P | F | chr2: 131129718- | Island |
| | | | | | 131130511 | |
| cg27053763 | chr5: 180653815 | TRIM41.GENEBODY | SCLC-P | R | chr5: 180650099- | S_Shelf |
| | | | | | 180650679 | |
| cg27222157 | chr11: 66382978 | RBM14.TSS1500 | SCLC-P | F | chr11: 66383571- | N_Shore |
| | | | | | 66385047 | |
| cg27254046 | chr6: 57272398 | PRIM2.GENEBODY | SCLC-P | F | | |
| cg00854324 | chr16: 69941269 | WWP2.GENEBODY | SCLC-P | R | | |
| cg01099470 | chr2: 131103656 | IMP4.GENEBODY | SCLC-P | F | chr2: 131099469- | S_Shelf |
| | | | | | 131100863 | |
| cg02428072 | chr3: 169900473 | PHC3.TSS1500 | SCLC-P | F | chr3: 169898946- | S_Shore |
| | | | | | 169899626 | |
| cg03152913 | chr2: 178975712 | RBM45.TSS1500 | SCLC-P | R | chr2: 178977078- | N_Shore |
| | | | | | 178977828 | |
| cg03356778 | chr11: 120110544 | POU2F3.TSS1500 | SCLC-P | F | chr11: 120110497- | Island |
| | | | | | 120110719 | |
| cg04976330 | chr1: 31480640 | PUM1.GENEBODY | SCLC-P | R | | |
| cg05399330 | chr1: 55464530 | BSND.TSS200 | SCLC-P | R | chr1: 55462688- | S_Shore |
| | | | | | 55463010 | |
| cg06912922 | chr19: 12708229 | ZNF490.GENEBODY | SCLC-P | F | chr19: 12707696- | S_Shore |
| | | | | | 12708114 | |
| cg07993367 | chr7: 149568778 | ATP6VOE2.TSS1500 | SCLC-P | R | chr7: 149570366- | N_Shore |
| | | | | | 149571272 | |
| cg08313040 | chr6: 33092243 | HLA-DPB2.GENEBODY | SCLC-P | F | | |
| cg08320861 | chr6: 33095982 | HLA-DPB2.GENEBODY | SCLC-P | R | | |
| cg08651894 | chr1: 154832235 | KCNN3.GENEBODY | SCLC-P | R | | |
| cg09417011 | chr1: 54842404 | SSBP3.GENEBODY | SCLC-P | F | | |
| cg09735908 | chr16: 88770558 | RNF166.GENEBODY | SCLC-P | F | chr16: 88772237- | N_Shore |
| | | | | | 88773195 | |
| cg10833114 | chr12: 72246108 | MRS2P2.TSS1500 | SCLC-P | F | | |
| cg12022772 | chr2: 131130468 | PTPN18.GENEBODY | SCLC-P | R | chr2: 131129718- | Island |
| | | | | | 131130511 | |
| cg12598235 | chr15: 89921182 | LOC254559.TSS200 | SCLC-P | R | chr15: 89920793- | Island |
| | | | | | 89922768 | |
| cg15109449 | chr1: 12363259 | VPS13D.GENEBODY | SCLC-P | F | | |
| cg15618336 | chr12: 52182759 | SCN8A.GENEBODY | SCLC-P | R | | |
| cg15939973 | chr17: 76419841 | DNAH17.UTR3 | SCLC-P | F | chr17: 76421441- | N_Shore |
| | | | | | 76421766 | |
| cg17301216 | chr15: 89920348 | LOC254559.TSS1500 | SCLC-P | R | chr15: 89920793- | N_Shore |
| | | | | | 89922768 | |
| cg17730176 | chr17: 27404126 | TIAF1.TSS1500 | SCLC-P | R | chr17: 27406753- | N_Shelf |
| | | | | | 27408059 | |
| cg18942985 | chr2: 128523110 | WDR33.GENEBODY | SCLC-P | R | | |
| cg19465165 | chr2: 131130103 | PTPN18.GENEBODY | SCLC-P | R | chr2: 131129718- | Island |
| | | | | | 131130511 | |
| cg19856701 | chr17: 29549474 | NF1.UTR3 | SCLC-P | F | | |
| cg20886791 | chr6: 33096055 | HLA-DPB2.GENEBODY | SCLC-P | R | | |
| cg22158248 | chr12: 112204136 | ALDH2.TSS1500 | SCLC-P | R | chr12: 112204498- | N_Shore |
| | | | | | 112204979 | |
| cg26306289 | chr7: 33080571 | NT5C3.TSS200 | SCLC-P | F | | |
| cg27627524 | chr8: 131325625 | ASAP1.GENEBODY | SCLC-P | R | | |
| cg00848167 | chr15: 89921898 | LOC254559.GENEBODY | SCLC-P | F | chr15: 89920793- | Island |
| | | | | | 89922768 | |
| cg01227084 | chr19: 2774520 | SGTA.UTR5 | SCLC-P | R | chr19: 2774196-2774550 | Island |
| cg01685549 | chr6: 33092097 | HLA-DPB2.GENEBODY | SCLC-P | R | | |
| cg01767053 | chr16: 85678884 | KIAA0182.UTR5 | SCLC-P | R | chr16: 85674843- | S_Shelf |
| | | | | | 85675192 | |
| cg01951712 | chr6: 33081535 | HLA-DPB2.GENEBODY | SCLC-P | F | chr6: 33084740- | N_Shelf |
| | | | | | 33084995 | |
| cg02388468 | chr6: 33095664 | HLA-DPB2.GENEBODY | SCLC-P | F | | |
| cg02470179 | chr3: 31580476 | STT3B.GENEBODY | SCLC-P | F | | |
| cg03766449 | chr15: 89920918 | LOC254559.TSS1500 | SCLC-P | F | chr15: 89920793- | Island |
| | | | | | 89922768 | |
| cg03917138 | chr17: 70120182 | SOX9.GENEBODY | SCLC-P | F | chr17: 70120139- | Island |
| | | | | | 70120442 | |
| cg04918708 | chr1: 204057900 | SOX13.UTR5 | SCLC-P | F | | |
| cg05427639 | chr7: 33080496 | NT5C3.EXON1 | SCLC-P | R | | |
| cg06517794 | chr1: 78180274 | USP33.GENEBODY | SCLC-P | R | | |
| cg07586345 | chr19: 2774332 | SGTA.UTR5 | SCLC-P | R | chr19: 2774196-2774550 | Island |
| cg09787021 | chr1: 25988486 | MAN1C1.GENEBODY | SCLC-P | R | | |
| cg09825327 | chr1: 211503219 | TRAF5.UTR5 | SCLC-P | F | chr1: 211500151- | S_Shelf |
| | | | | | 211500636 | |
| cg10327769 | chr17: 43340243 | LOC100133991.GENEBODY | SCLC-P | F | chr17: 43339124- | S_Shore |
| | | | | | 43339832 | |
| cg10657965 | chr7: 101888125 | CUX1.GENEBODY | SCLC-P | R | chr7: 101891665- | N_Shelf |
| | | | | | 101892634 | |
| cg11786476 | chr6: 33096738 | HLA-DPB2.GENEBODY | SCLC-P | R | | |
| cg12406391 | chr1: 6520194 | ESPN.GENEBODY | SCLC-P | F | chr1: 6520584-6521955 | N_Shore |
| cg12873454 | chr1: 14924611 | KIAA1026.TSS1500 | SCLC-P | R | chr1: 14924610- | Island |
| | | | | | 14925993 | |
| cg13077784 | chr1: 201706363 | NAV1.GENEBODY | SCLC-P | R | chr1: 201708787- | N_Shelf |
| | | | | | 201709429 | |
| cg13977526 | chr9: 32384244 | ACO1.TSS1500 | SCLC-P | F | chr9: 32384420- | N_Shore |
| | | | | | 32384911 | |
| cg15019001 | chr6: 33096303 | HLA-DPB2.GENEBODY | SCLC-P | R | | |
| cg15826519 | chr1: 19808575 | CAPZB.GENEBODY | SCLC-P | F | chr1: 19811703- | N_Shelf |
| | | | | | 19812644 | |
| cg16072780 | chr11: 93453897 | SCARNA9.TSS1500 | SCLC-P | F | | |
| cg16205850 | chr15: 75118021 | LMAN1L.UTR3 | SCLC-P | R | | |
| cg18109571 | chr6: 127835189 | C6orf174.GENEBODY | SCLC-P | F | chr6: 127835928- | N_Shore |
| | | | | | 127836255 | |
| cg18198143 | chr6: 33094306 | HLA-DPB2.GENEBODY | SCLC-P | F | | |
| cg18474502 | chr10: 124777194 | ACADSB.GENEBODY | SCLC-P | R | | |
| cg18680328 | chr10: 123868970 | TACC2.GENEBODY | SCLC-P | R | chr10: 123872119- | N_Shelf |
| | | | | | 123873462 | |
| cg19046783 | chr7: 99382086 | CYP3A4.TSS1500 | SCLC-P | R | | |
| cg20314331 | chr2: 131129567 | PTPN18.GENEBODY | SCLC-P | R | chr2: 131129718- | N_Shore |
| | | | | | 131130511 | |
| cg20567355 | chr8: 52754434 | PCMTD1.GENEBODY | SCLC-P | R | | |
| cg21516291 | chr20: 44979100 | SLC35C2.GENEBODY | SCLC-P | F | | |
| cg21955534 | chrX: 15525514 | BMX.EXON1 | SCLC-P | F | | |
| cg22052010 | chr20: 33541934 | GSS.UTR5 | SCLC-P | R | chr20: 33543082- | N_Shore |
| | | | | | 33543846 | |
| cg22888311 | chr12: 80076443 | PAWR.GENEBODY | SCLC-P | F | | |
| cg23538755 | chr19: 2774549 | SGTA.UTR5 | SCLC-P | F | chr19: 2774196-2774550 | Island |
| cg24156613 | chrX: 39950501 | BCOR.UTR5 | SCLC-P | R | chrX: 39950899- | N_Shore |
| | | | | | 39951665 | |
| cg24180219 | chr10: 105239190 | CALHM3.TSS200 | SCLC-P | R | | |
| cg25064331 | chr7: 33080500 | NT5C3.EXON1 | SCLC-P | R | | |
| cg25474399 | chr18: 3067624 | MYOM1.GENEBODY | SCLC-P | F | chr18: 3067297-3067533 | S_Shore |
| cg25759507 | chr11: 12008553 | DKK3.GENEBODY | SCLC-P | R | | |
| cg26086731 | chr3: 169900468 | PHC3.TSS1500 | SCLC-P | F | chr3: 169898946- | S_Shore |
| | | | | | 169899626 | |
| cg26469011 | chr10: 75886930 | AP3M1.GENEBODY | SCLC-P | F | | |
| cg26536240 | chr16: 89509760 | ANKRD11.UTR5 | SCLC-P | R | | |
| cg00350405 | chr2: 128244940 | IWS1.GENEBODY | SCLC-P | F | | |
| cg01541650 | chr6: 157505968 | ARID1B.GENEBODY | SCLC-P | R | | |
| cg02061660 | chr5: 110088130 | SLC25A46.GENEBODY | SCLC-P | F | | |
| cg02380236 | chr15: 22383188 | OR4N4.EXON1 | SCLC-P | F | | |
| cg03233656 | chr2: 65214625 | SLC1A4.TSS1500 | SCLC-P | F | chr2: 65215598- | N_Shore |
| | | | | | 65217212 | |
| cg04824189 | chr7: 80142634 | GNAT3.TSS1500 | SCLC-P | F | | |
| cg05009111 | chr20: 58520201 | C20orf177.UTR3 | SCLC-P | R | | |
| cg05366160 | chr6: 33039481 | HLA-DPA1.GENEBODY | SCLC-P | F | | |
| cg06614640 | chr17: 80826697 | TBCD.GENEBODY | SCLC-P | R | chr17: 80827970- | N_Shore |
| | | | | | 80828316 | |
| cg09142647 | chr16: 75674568 | KARS.GENEBODY | SCLC-P | R | | |
| cg10094078 | chr19: 1467925 | APC2.GENEBODY | SCLC-P | F | chr19: 1465206-1471241 | Island |
| cg10297701 | chr6: 32972902 | HLA-DOA.UTR3 | SCLC-P | F | chr6: 32975684- | N_Shelf |
| | | | | | 32975926 | |
| cg10604002 | chr2: 131130131 | PTPN18.GENEBODY | SCLC-P | R | chr2: 131129718- | Island |
| | | | | | 131130511 | |
| cg10725390 | chr2: 66743000 | MEIS1.GENEBODY | SCLC-P | F | | |
| cg12733040 | chr22: 17303493 | XKR3.TSS1500 | SCLC-P | F | | |
| cg12748948 | chr1: 22106295 | USP48.GENEBODY | SCLC-P | F | chr1: 22109129- | N_Shelf |
| | | | | | 22110261 | |
| cg12815916 | chr2: 131114432 | PTPN18.GENEBODY | SCLC-P | F | chr2: 131113090- | S_Shore |
| | | | | | 131114277 | |
| cg13726789 | chr8: 37678323 | GPR124.GENEBODY | SCLC-P | F | | |
| cg13922121 | chr11: 19575262 | NAV2.GENEBODY | SCLC-P | F | | |
| cg15214229 | chr20: 35275274 | SLA2.TSS1500 | SCLC-P | R | | |
| cg16296829 | chr1: 154832535 | KCNN3.TSS200 | SCLC-P | R | | |
| cg16431810 | chr7: 2420339 | EIF3B.UTR3 | SCLC-P | F | chr7: 2419808-2420340 | Island |
| cg18451156 | chr12: 133424373 | CHFR.GENEBODY | SCLC-P | R | | |
| cg18963065 | chrX: 15525321 | BMX.UTR5 | SCLC-P | F | | |
| cg21049501 | chr17: 70120411 | SOX9.GENEBODY | SCLC-P | F | chr17: 70120139- | Island |
| | | | | | 70120442 | |
| cg21121843 | chr4: 3203982 | HTT.GENEBODY | SCLC-P | R | | |
| cg21156320 | chr12: 56112820 | BLOC1S1.GENEBODY | SCLC-P | R | chr12: 56109798- | S_Shelf |
| | | | | | 56110298 | |
| cg04403809 | chr20: 44686493 | SLC12A5.UTR3 | SCLC-P | R | chr20: 44685771- | Island |
| | | | | | 44687610 | |
| cg00538021 | chr16: 12192263 | SNX29.GENEBODY | SCLC-P | R | | |
| cg00712146 | chr3: 65340538 | MAGI1.UTR3 | SCLC-P | R | chr3: 65342203- | N_Shore |
| | | | | | 65342884 | |
| cg01249653 | chr6: 33095755 | HLA-DPB2.GENEBODY | SCLC-P | F | | |
| cg03212228 | chr19: 38395481 | WDR87.UTR5 | SCLC-P | F | chr19: 38397003- | N_Shore |
| | | | | | 38398165 | |
| cg03387092 | chr3: 123493248 | MYLK.GENEBODY | SCLC-P | R | | |
| cg04117530 | chr1: 226187852 | C1orf55.TSS1500 | SCLC-P | R | chr1: 226186803- | S_Shore |
| | | | | | 226187336 | |
| cg04256886 | chr2: 25603640 | DTNB.GENEBODY | SCLC-P | R | chr2: 25599676- | S_Shelf |
| | | | | | 25599895 | |
| cg04351807 | chr11: 36013639 | LDLRAD3.GENEBODY | SCLC-P | R | | |
| cg04478905 | chr21: 37915091 | CLDN14.TSS200 | SCLC-P | F | | |
| cg04998498 | chr11: 113725687 | USP28.GENEBODY | SCLC-P | F | | |
| cg05613017 | chr12: 65589446 | LEMD3.GENEBODY | SCLC-P | F | | |
| cg06234051 | chr17: 70120541 | SOX9.UTR3 | SCLC-P | F | chr17: 70116274- | S_Shore |
| | | | | | 70119998 | |
| cg06532924 | chr1: 247268238 | ZNF669.TSS1500 | SCLC-P | F | chr1: 247267179- | S_Shore |
| | | | | | 247267884 | |
| cg07069844 | chr19: 1617027 | TCF3.GENEBODY | SCLC-P | F | chr19: 1615202-1615812 | S_Shore |
| cg07202461 | chr18: 19406765 | MIB1.GENEBODY | SCLC-P | F | | |
| cg07813851 | chr1: 7466976 | CAMTA1.GENEBODY | SCLC-P | F | | |
| cg08088222 | chr12: 122070432 | ORAI1.GENEBODY | SCLC-P | F | | |
| cg08948637 | chr20: 62614493 | PRPF6.GENEBODY | SCLC-P | R | chr20: 62612300- | S_Shore |
| | | | | | 62612787 | |
| cg10004079 | chr17: 26372530 | NLK.GENEBODY | SCLC-P | R | chr17: 26368586- | S_Shelf |
| | | | | | 26369133 | |
| cg10735599 | chr6: 33081024 | HLA-DPB2.GENEBODY | SCLC-P | F | chr6: 33084740- | N_Shelf |
| | | | | | 33084995 | |
| cg10932086 | chr14: 90041348 | PRO1768.TSS1500 | SCLC-P | F | | |
| cg12255995 | chr19: 1132537 | SBNO2.GENEBODY | SCLC-P | R | | |
| cg12555369 | chr10: 105153066 | USMG5.UTR5 | SCLC-P | R | chr10: 105155820- | N_Shelf |
| | | | | | 105156484 | |
| cg12577003 | chr7: 2261337 | MAD1L1.GENEBODY | SCLC-P | R | | |
| cg12582616 | chr6: 15427489 | JARID2.GENEBODY | SCLC-P | F | | |
| cg13058710 | chr17: 70119609 | SOX9.GENEBODY | SCLC-P | F | chr17: 70116274- | Island |
| | | | | | 70119998 | |
| cg13362033 | chr1: 7018949 | CAMTA1.GENEBODY | SCLC-P | F | chr1: 7022001-7022308 | N_Shelf |
| cg13726166 | chr17: 27403888 | TIAF1.TSS1500 | SCLC-P | F | chr17: 27406753- | N_Shelf |
| | | | | | 27408059 | |
| cg13738075 | chr13: 114793125 | RASA3.GENEBODY | SCLC-P | R | | |
| cg14373068 | chr11: 1537765 | HCCA2.GENEBODY | SCLC-P | F | chr11: 1536729-1536953 | S_Shore |
| cg14521421 | chr11: 8862019 | ST5.UTR5 | SCLC-P | R | | |
| cg14762436 | chr7: 24917750 | OSBPL3.GENEBODY | SCLC-P | F | | |
| cg17223866 | chr1: 44794158 | ERI3.GENEBODY | SCLC-P | F | | |
| cg18236734 | chr3: 183817931 | HTR3E.TSS200 | SCLC-P | F | | |
| cg18714586 | chr13: 30831499 | KATNAL1.GENEBODY | SCLC-P | R | | |
| cg19232395 | chr11: 8550221 | STK33.UTR5 | SCLC-P | R | | |
| cg19251161 | chr10: 106094166 | ITPRIP.TSS1500 | SCLC-P | R | chr10: 106097595- | N_Shelf |
| | | | | | 106098556 | |
| cg19490899 | chr6: 52855923 | GSTA4.GENEBODY | SCLC-P | R | chr6: 52859565- | N_Shelf |
| | | | | | 52859767 | |
| cg19644145 | chr7: 21488742 | SP4.GENEBODY | SCLC-P | F | | |
| cg20288892 | chr7: 82072332 | CACNA2D1.GENEBODY | SCLC-P | R | chr7: 82072021- | Island |
| | | | | | 82073520 | |
| cg20706496 | chr6: 112084071 | FYN.UTR5 | SCLC-P | R | | |
| cg22235258 | chr11: 34675402 | EHF.GENEBODY | SCLC-P | F | | |
| cg22736267 | chr7: 148841524 | ZNF398.UTR5 | SCLC-P | F | chr7: 148844254- | N_Shelf |
| | | | | | 148845556 | |
| cg23025804 | chr10: 29811659 | SVIL.GENEBODY | SCLC-P | F | | |
| cg23794671 | chr6: 32977580 | HLA-DOA.TSS200 | SCLC-P | F | chr6: 32975684- | S_Shore |
| | | | | | 32975926 | |
| cg23998903 | chr2: 209048830 | C2orf80.GENEBODY | SCLC-P | F | | |
| cg24223075 | chr11: 119137279 | CBL.GENEBODY | SCLC-P | F | | |
| cg24321688 | chr19: 39393347 | NFKBIB.GENEBODY | SCLC-P | F | chr19: 39395881- | N_Shelf |
| | | | | | 39396106 | |
| cg24904278 | chr4: 3514709 | LRPAP1.GENEBODY | SCLC-P | F | chr4: 3514537-3514848 | Island |
| cg24918798 | chr2: 103432687 | TMEM182.UTR3 | SCLC-P | R | | |
| cg25380514 | chr3: 169900418 | PHC3.TSS1500 | SCLC-P | F | chr3: 169898946- | S_Shore |
| | | | | | 169899626 | |
| cg25649889 | chr2: 171196296 | MYO3B.GENEBODY | SCLC-P | F | | |
| cg25681122 | chr3: 194832600 | C3orf21.GENEBODY | SCLC-P | F | | |
| cg25848827 | chr1: 7550111 | CAMTA1.GENEBODY | SCLC-P | R | | |
| cg25977504 | chr1: 247979157 | OR14A16.TSS200 | SCLC-P | R | | |
| cg00425764 | chr16: 15949616 | MYH11.UTR5 | SCLC-P | R | chr16: 15950839- | N_Shore |
| | | | | | 15951266 | |
| cg00863378 | chr16: 56549757 | BBS2.GENEBODY | SCLC-P | R | chr16: 56553529- | N_Shelf |
| | | | | | 56554075 | |
| cg00946598 | chr1: 44117093 | KDM4A.UTR5 | SCLC-P | F | chr1: 44115616- | S_Shore |
| | | | | | 44116309 | |
| cg01140395 | chr15: 52570565 | MIR1266.TSS1500 | SCLC-P | F | | |
| cg01418162 | chr1: 204891567 | NFASC.UTR5 | SCLC-P | R | | |
| cg01708014 | chr14: 51299002 | NIN.TSS1500 | SCLC-P | R | chr14: 51296727- | S_Shore |
| | | | | | 51298477 | |
| cg01772439 | chr5: 149012102 | FLJ41603.UTR3 | SCLC-P | R | | |
| cg01859360 | chr17: 26990458 | SUPT6H.UTR5 | SCLC-P | R | chr17: 26988721- | S_Shore |
| | | | | | 26989391 | |
| cg01891209 | chr1: 44401222 | ARTN.TSS1500 | SCLC-P | R | chr1: 44401757- | N_Shore |
| | | | | | 44402423 | |
| cg02362718 | chr11: 133942581 | JAM3.GENEBODY | SCLC-P | F | chr11: 133938850- | S_Shelf |
| | | | | | 133939681 | |
| cg02632809 | chr7: 5336811 | SLC29A4.GENEBODY | SCLC-P | R | chr7: 5336513-5336894 | Island |
| cg04022912 | chr16: 24864984 | SLC5A11.UTR5 | SCLC-P | F | | |
| cg04153082 | chr8: 48325740 | KIAA0146.GENEBODY | SCLC-P | F | | |
| cg04272613 | chr14: 59721173 | DAAM1.UTR5 | SCLC-P | F | | |
| cg04694619 | chr2: 242127759 | ANO7.TSS200 | SCLC-P | F | | |
| cg05995528 | chr6: 33036495 | HLA-DPA1.GENEBODY | SCLC-P | F | | |
| cg06821199 | chr4: 779230 | CPLX1.UTR3 | SCLC-P | F | chr4: 778661-780592 | Island |
| cg06985721 | chr1: 55464653 | BSND.EXON1 | SCLC-P | F | chr1: 55462688- | S_Shore |
| | | | | | 55463010 | |
| cg08961287 | chr15: 89919993 | LOC254559.TSS1500 | SCLC-P | F | chr15: 89920793- | N_Shore |
| | | | | | 89922768 | |
| cg09561830 | chr13: 100007116 | UBAC2.GENEBODY | SCLC-P | R | | |
| cg10042298 | chr11: 78024311 | GAB2.GENEBODY | SCLC-P | R | | |
| cg10471574 | chr17: 70120318 | SOX9.GENEBODY | SCLC-P | F | chr17: 70120139- | Island |
| | | | | | 70120442 | |
| cg10803321 | chr1: 50629523 | ELAVL4.GENEBODY | SCLC-P | R | | |
| cg10818986 | chr1: 248083146 | OR2T8.TSS1500 | SCLC-P | R | | |
| cg11131599 | chr1: 204224211 | PLEKHA6.GENEBODY | SCLC-P | F | chr1: 204226544- | N_Shelf |
| | | | | | 204226908 | |
| cg12338214 | chr10: 133783113 | BNIP3.GENEBODY | SCLC-P | R | | |
| cg12499542 | chr12: 53808156 | SP1.UTR3 | SCLC-P | R | | |
| cg12832726 | chr11: 68096165 | LRP5.GENEBODY | SCLC-P | F | | |
| cg13375479 | chr7: 133984347 | SLC35B4.GENEBODY | SCLC-P | F | | |
| cg13551330 | chr9: 32388149 | ACO1.UTR5 | SCLC-P | R | chr9: 32384420- | S_Shelf |
| | | | | | 32384911 | |
| cg13696135 | chr12: 49396782 | PRKAG1.GENEBODY | SCLC-P | R | chr12: 49393139- | S_Shelf |
| | | | | | 49393361 | |
| cg14200678 | chr9: 123380343 | MEGF9.GENEBODY | SCLC-P | F | | |
| cg14373797 | chr6: 33053576 | HLA-DPB1.GENEBODY | SCLC-P | R | | |
| cg14880894 | chr4: 78697859 | CNOT6L.GENEBODY | SCLC-P | R | | |
| cg15661715 | chr7: 151401947 | PRKAG2.GENEBODY | SCLC-P | F | | |
| cg15955522 | chr5: 143553445 | KCTD16.UTR5 | SCLC-P | R | chr5: 143549939- | S_Shelf |
| | | | | | 143550333 | |
| cg16083838 | chr11: 2889809 | KCNQ1DN.TSS1500 | SCLC-P | R | chr11: 2890388-2891337 | N_Shore |
| cg16189995 | chr2: 226330770 | KIAA1486.GENEBODY | SCLC-P | R | | |
| cg17491228 | chr11: 64476089 | NRXN2.GENEBODY | SCLC-P | F | chr11: 64478843- | N_Shelf |
| | | | | | 64479598 | |
| cg17851748 | chr12: 83264140 | TMTC2.GENEBODY | SCLC-P | R | | |
| cg18063824 | chr4: 54075042 | SCFD2.GENEBODY | SCLC-P | F | | |
| cg18282011 | chr1: 1590447 | CDK11B.GENEBODY | SCLC-P | F | chr1: 1590073-1590942 | Island |
| cg19083007 | chr6: 46293862 | RCAN2.TSS1500 | SCLC-P | R | | |
| cg19740679 | chr1: 21930180 | RAP1GAP.GENEBODY | SCLC-P | R | chr1: 21930179- | Island |
| | | | | | 21930380 | |
| cg20535805 | chr19: 18420735 | LSM4.GENEBODY | SCLC-P | F | chr19: 18420489- | Island |
| | | | | | 18420736 | |
| cg20643029 | chr21: 37915044 | CLDN14.TSS200 | SCLC-P | F | | |
| cg21039196 | chr6: 71435620 | SMAP1.GENEBODY | SCLC-P | F | | |
| cg21221516 | chr3: 183950129 | VWA5B2.GENEBODY | SCLC-P | F | chr3: 183947040- | S_Shore |
| | | | | | 183948801 | |
| cg21350778 | chr17: 64297603 | PRKCA.TSS1500 | SCLC-P | R | chr17: 64298047- | N_Shore |
| | | | | | 64299617 | |
| cg23034373 | chr21: 37915354 | CLDN14.UTR5 | SCLC-P | F | | |
| cg23200572 | chr6: 33096501 | HLA-DPB2.GENEBODY | SCLC-P | R | | |
| cg24008634 | chr10: 13694895 | FRMD4A.GENEBODY | SCLC-P | R | chr10: 13698562- | N_Shelf |
| | | | | | 13699540 | |
| cg24401199 | chr19: 3159786 | GNA15.GENEBODY | SCLC-P | F | chr19: 3162841-3163161 | N_Shelf |
| cg24677302 | chr14: 75922163 | JDP2.GENEBODY | SCLC-P | R | | |
| cg24987745 | chr2: 17819167 | VSNL1.GENEBODY | SCLC-P | R | | |
| cg25119261 | chr6: 33081351 | HLA-DPB2.GENEBODY | SCLC-P | R | chr6: 33084740- | N_Shelf |
| | | | | | 33084995 | |
| cg25243171 | chr2: 23914356 | KLHL29.GENEBODY | SCLC-P | R | chr2: 23913308- | S_Shore |
| | | | | | 23913569 | |
| cg26886855 | chr8: 10694831 | PINX1.GENEBODY | SCLC-P | R | chr8: 10697016- | N_Shelf |
| | | | | | 10697634 | |
| cg26949694 | chr11: 27742060 | BDNF.GENEBODY | SCLC-P | R | chr11: 27742059- | Island |
| | | | | | 27742273 | |
| cg27636594 | chr7: 38767436 | VPS41.GENEBODY | SCLC-P | R | | |
| cg01208614 | chr5: 141031443 | FCHSD1.TSS1500 | SCLC-P | R | chr5: 141030918- | S_Shore |
| | | | | | 141031177 | |
| cg02389877 | chr15: 68583600 | FEM1B.UTR3 | SCLC-P | F | | |
| cg02882004 | chr2: 201334663 | SPATS2L.GENEBODY | SCLC-P | R | | |
| cg04077303 | chr1: 236308094 | GPR137B.GENEBODY | SCLC-P | R | chr1: 236305534- | S_Shore |
| | | | | | 236306674 | |
| cg04576847 | chr17: 12623611 | MYOCD.GENEBODY | SCLC-P | R | | |
| cg06879534 | chr4: 39447437 | KLB.GENEBODY | SCLC-P | F | chr4: 39448184- | N_Shore |
| | | | | | 39449054 | |
| cg07010146 | chr10: 105239205 | CALHM3.TSS1500 | SCLC-P | R | | |
| cg07304186 | chr6: 38112278 | ZFAND3.GENEBODY | SCLC-P | R | | |
| cg08759770 | chr12: 51849767 | SLC4A8.GENEBODY | SCLC-P | F | | |
| cg13339454 | chr2: 242127690 | ANO7.TSS1500 | SCLC-P | F | | |
| cg14080888 | chr9: 130712990 | FAM102A.UTR5 | SCLC-P | F | | |
| cg18477771 | chr11: 120195466 | TMEM136.TSS1500 | SCLC-P | F | chr11: 120195805- | N_Shore |
| | | | | | 120196372 | |
| cg19377250 | chr7: 100463206 | SLC12A9.GENEBODY | SCLC-P | F | chr7: 100463758- | N_Shore |
| | | | | | 100464146 | |
| cg20272287 | chr17: 19247905 | MIR1180.TSS200 | SCLC-P | F | chr17: 19247785- | Island |
| | | | | | 19248069 | |
| cg21265278 | chr1: 179057067 | TOR3A.GENEBODY | SCLC-P | F | | |
| cg23541975 | chr17: 75143184 | SEC14L1.GENEBODY | SCLC-P | R | | |
| cg00552822 | chr15: 22368443 | LOC727924.GENEBODY | SCLC-P | R | | |
| cg00796611 | chr1: 206603444 | SRGAP2.GENEBODY | SCLC-P | R | | |
| cg01367339 | chr3: 44962718 | ZDHHC3.GENEBODY | SCLC-P | R | | |
| cg01638185 | chr17: 64530027 | PRKCA.GENEBODY | SCLC-P | R | | |
| cg01668281 | chr21: 37915281 | CLDN14.UTR5 | SCLC-P | R | | |
| cg01715699 | chr13: 99209655 | STK24.GENEBODY | SCLC-P | R | | |
| cg01854491 | chr8: 144815021 | FAM83H.UTR5 | SCLC-P | F | chr8: 144815203- | N_Shore |
| | | | | | 144816518 | |
| cg02246992 | chr17: 29158363 | ATAD5.TSS1500 | SCLC-P | F | chr17: 29158878- | N_Shore |
| | | | | | 29159423 | |
| cg02370478 | chrX: 106241676 | MORC4.GENEBODY | SCLC-P | R | chrX: 106242506- | N_Shore |
| | | | | | 106243593 | |
| cg02544829 | chr8: 95385408 | RAD54B.GENEBODY | SCLC-P | F | | |
| cg03332109 | chr11: 126360468 | KIRREL3.GENEBODY | SCLC-P | F | | |
| cg04086197 | chr14: 104205071 | PPP1R13B.GENEBODY | SCLC-P | R | chr14: 104205070- | Island |
| | | | | | 104205329 | |
| cg04236786 | chr8: 41580903 | ANK1.GENEBODY | SCLC-P | R | chr8: 41583135- | N_Shelf |
| | | | | | 41583524 | |
| cg04920416 | chr17: 35336293 | AATF.GENEBODY | SCLC-P | R | | |
| cg05490757 | chr17: 77130342 | HRNBP3.UTR5 | SCLC-P | F | chr17: 77133927- | N_Shelf |
| | | | | | 77134175 | |
| cg06927775 | chr17: 40609423 | ATP6VOA1.TSS1500 | SCLC-P | R | chr17: 40610746- | N_Shore |
| | | | | | 40611032 | |
| cg07481360 | chr3: 196902551 | DLG1.GENEBODY | SCLC-P | F | | |
| cg07535740 | chr19: 5915494 | CAPS.UTR3 | SCLC-P | F | chr19: 5915007-5915301 | S_Shore |
| cg08707617 | chrX: 39944352 | BCOR.UTR5 | SCLC-P | R | chrX: 39942756- | S_Shore |
| | | | | | 39943419 | |
| cg08760402 | chr6: 33036486 | HLA-DPA1.GENEBODY | SCLC-P | R | | |
| cg09002351 | chr16: 75204945 | ZFP1.UTR3 | SCLC-P | F | | |
| cg09203240 | chr11: 68115891 | LRP5.GENEBODY | SCLC-P | R | chr11: 68115317- | S_Shore |
| | | | | | 68115726 | |
| cg10674511 | chrX: 78003091 | LPAR4.TSS200 | SCLC-P | F | | |
| cg10808783 | chr19: 1467921 | APC2.GENEBODY | SCLC-P | F | chr19: 1465206-1471241 | Island |
| cg11336987 | chr3: 123974913 | KALRN.GENEBODY | SCLC-P | F | | |
| cg12700788 | chr17: 72348160 | KIF19.GENEBODY | SCLC-P | R | chr17: 72347924- | Island |
| | | | | | 72348322 | |
| cg12892471 | chr12: 50616779 | LIMA1.GENEBODY | SCLC-P | F | | |
| cg13356324 | chr1: 228332502 | GUK1.UTR5 | SCLC-P | R | chr1: 228327508- | S_Shelf |
| | | | | | 228328560 | |
| cg14353201 | chr11: 67448395 | ALDH3B2.UTR5 | SCLC-P | F | | |
| cg15770046 | chr19: 35764710 | USF2.GENEBODY | SCLC-P | F | chr19: 35760457- | S_Shelf |
| | | | | | 35760865 | |
| cg16712103 | chr1: 193150499 | B3GALT2.GENEBODY | SCLC-P | F | | |
| cg16831185 | chr17: 29158530 | ATAD5.TSS1500 | SCLC-P | F | chr17: 29158878- | N_Shore |
| | | | | | 29159423 | |
| cg17219729 | chr1: 36937845 | CSF3R.GENEBODY | SCLC-P | F | | |
| cg17582782 | chr15: 57592438 | LOC283663.TSS200 | SCLC-P | F | | |
| cg17649671 | chr10: 89298596 | MINPP1.GENEBODY | SCLC-P | F | | |
| cg18106482 | chr11: 2680314 | KCNQ1.GENEBODY | SCLC-P | R | | |
| cg18993757 | chr17: 4889754 | CAMTA2.UTR5 | SCLC-P | F | chr17: 4890282-4890991 | N_Shore |
| cg19113599 | chr15: 99683042 | TTC23.GENEBODY | SCLC-P | R | | |
| cg19296405 | chr19: 18368483 | KIAA1683.GENEBODY | SCLC-P | R | chr19: 18368482- | Island |
| | | | | | 18368911 | |
| cg19533977 | chr17: 57719682 | CLTC.GENEBODY | SCLC-P | F | | |
| cg19601328 | chr17: 43368729 | MAP3K14.UTR5 | SCLC-P | R | | |
| cg19909036 | chr15: 90294776 | MESP1.TSS1500 | SCLC-P | F | chr15: 90293221- | S_Shore |
| | | | | | 90294733 | |
| cg20664654 | chrX: 153169465 | AVPR2.TSS1500 | SCLC-P | R | | |
| cg22057240 | chr5: 6755843 | POLS.UTR3 | SCLC-P | R | chr5: 6755104-6755387 | S_Shore |
| cg22675922 | chr11: 2889840 | KCNQ1DN.TSS1500 | SCLC-P | R | chr11: 2890388-2891337 | N_Shore |
| cg24051242 | chr11: 68096138 | LRP5.GENEBODY | SCLC-P | R | | |
| cg24063043 | chr17: 64567021 | PRKCA.GENEBODY | SCLC-P | R | | |
| cg24420432 | chr7: 101518619 | CUX1.GENEBODY | SCLC-P | F | | |
| cg27050612 | chr17: 46133198 | NFE2L1.GENEBODY | SCLC-P | F | chr17: 46136649- | N_Shelf |
| | | | | | 46136969 | |
| cg27064928 | chrX: 146996911 | ASFMR1.GENEBODY | SCLC-P | F | chrX: 146993091- | S_Shelf |
| | | | | | 146994200 | |
| cg01966522 | chr3: 126236504 | UROC1.EXON1 | SCLC-P | R | | |
| cg04619304 | chr12: 24434537 | SOX5.UTR5 | SCLC-P | R | | |
| cg05043047 | chr8: 144583910 | ZC3H3.GENEBODY | SCLC-P | R | | |
| cg05169160 | chr2: 178484487 | TTC30A.TSS1500 | SCLC-P | R | chr2: 178483233- | S_Shore |
| | | | | | 178483783 | |
| cg05733361 | chr15: 85524456 | PDE8A.TSS1500 | SCLC-P | R | chr15: 85523512- | Island |
| | | | | | 85525865 | |
| cg06895124 | chr8: 141588750 | EIF2C2.GENEBODY | SCLC-P | R | | |
| cg07178896 | chr16: 50238554 | PAPD5.GENEBODY | SCLC-P | F | | |
| cg09038267 | chr10: 104524861 | C10orf26.GENEBODY | SCLC-P | F | | |
| cg10369242 | chr12: 52625486 | KRT7.TSS1500 | SCLC-P | R | chr12: 52626793- | N_Shore |
| | | | | | 52627577 | |
| cg11678118 | chr6: 122935372 | PKIB.UTR5 | SCLC-P | F | chr6: 122931153- | S_Shelf |
| | | | | | 122931705 | |
| cg12100006 | chr11: 35964190 | LDLRAD3.TSS1500 | SCLC-P | R | chr11: 35965642- | N_Shore |
| | | | | | 35966454 | |
| cg12738079 | chrX: 153169469 | AVPR2.TSS1500 | SCLC-P | R | | |
| cg12745145 | chr7: 2261452 | MAD1L1.GENEBODY | SCLC-P | F | | |
| cg13259357 | chr3: 191109928 | CCDC50.UTR3 | SCLC-P | F | | |
| cg14356799 | chr6: 33044345 | HLA-DPB1.GENEBODY | SCLC-P | R | | |
| cg14981137 | chr5: 50678697 | ISL1.TSS1500 | SCLC-P | R | chr5: 50678632- | Island |
| | | | | | 50679375 | |
| cg16235962 | chr11: 118754507 | CXCR5.TSS200 | SCLC-P | F | | |
| cg18907656 | chr8: 30888925 | PURG.GENEBODY | SCLC-P | F | | |
| cg19407960 | chr6: 33094829 | HLA-DPB2.GENEBODY | SCLC-P | F | | |
| cg20738500 | chr15: 89921156 | LOC254559.TSS200 | SCLC-P | F | chr15: 89920793- | Island |
| | | | | | 89922768 | |
| cg20851325 | chrX: 153169432 | AVPR2.TSS1500 | SCLC-P | F | | |
| cg21211730 | chr19: 45279770 | CBLC.TSS1500 | SCLC-P | F | chr19: 45281133- | N_Shore |
| | | | | | 45281355 | |
| cg21608489 | chr19: 42637137 | POU2F2.TSS1500 | SCLC-P | R | chr19: 42637277- | N_Shore |
| | | | | | 42637684 | |
| cg22338118 | chr7: 94536003 | PPP1R9A.TSS1500 | SCLC-P | F | chr7: 94536849- | N_Shore |
| | | | | | 94537477 | |
| cg24644448 | chr2: 114383268 | RPL23AP7.GENEBODY | SCLC-P | R | chr2: 114384589- | N_Shore |
| | | | | | 114385133 | |
| cg25694790 | chr20: 17582752 | DSTN.GENEBODY | SCLC-P | F | | |
| cg26067647 | chr19: 1420404 | DAZAP1.GENEBODY | SCLC-P | F | chr19: 1419751-1420405 | Island |
| cg26643856 | chr6: 32977349 | HLA-DOA.EXON1 | SCLC-P | R | chr6: 32975684- | S_Shore |
| | | | | | 32975926 | |
| cg27500983 | chr20: 34700201 | EPB41L1.TSS200 | SCLC-P | R | | |
| cg00166343 | chr17: 29150100 | CRLF3.GENEBODY | SCLC-P | R | chr17: 29151481- | N_Shore |
| | | | | | 29151901 | |
| cg01091565 | chr15: 90294762 | MESP1.TSS1500 | SCLC-P | F | chr15: 90293221- | S_Shore |
| | | | | | 90294733 | |
| cg01755009 | chr1: 247978690 | OR14A16.EXON1 | SCLC-P | F | | |
| cg02347367 | chr6: 109250017 | ARMC2.GENEBODY | SCLC-P | R | | |
| cg02397571 | chr8: 28175463 | PNOC.UTR5 | SCLC-P | F | | |
| cg06544652 | chr1: 248006259 | OR11L1.TSS1500 | SCLC-P | R | | |
| cg08097973 | chr11: 71793051 | LRTOMT.UTR5 | SCLC-P | R | chr11: 71791385- | S_Shore |
| | | | | | 71791960 | |
| cg08822075 | chr7: 26193607 | NFE2L3.GENEBODY | SCLC-P | R | chr7: 26191794- | S_Shore |
| | | | | | 26192757 | |
| cg10057033 | chr8: 41737893 | ANK1.GENEBODY | SCLC-P | F | chr8: 41733535- | S_Shelf |
| | | | | | 41733911 | |
| cg11301657 | chr12: 3749918 | EFCAB4B.GENEBODY | SCLC-P | R | | |
| cg11437465 | chr6: 33036958 | HLA-DPA1.GENEBODY | SCLC-P | F | | |
| cg12704098 | chr3: 56838665 | ARHGEF3.GENEBODY | SCLC-P | F | chr3: 56835650- | S_Shelf |
| | | | | | 56836210 | |
| cg15642209 | chr17: 66290505 | ARSG.UTR5 | SCLC-P | R | chr17: 66286541- | S_Shore |
| | | | | | 66288532 | |
| cg15841865 | chr7: 143173551 | TAS2R41.TSS1500 | SCLC-P | F | | |
| cg16027403 | chr1: 247978791 | OR14A16.EXON1 | SCLC-P | F | | |
| cg16527552 | chr19: 42816045 | TMEM145.TSS1500 | SCLC-P | R | chr19: 42817412- | N_Shore |
| | | | | | 42818038 | |
| cg17138415 | chr12: 107259568 | RIC8B.GENEBODY | SCLC-P | R | | |
| cg17395184 | chr15: 42750462 | ZFP106.TSS1500 | SCLC-P | R | | |
| cg19333963 | chr19: 1467979 | APC2.GENEBODY | SCLC-P | F | chr19: 1465206-1471241 | Island |
| cg19494588 | chr5: 146195103 | PPP2R2B.GENEBODY | SCLC-P | R | | |
| cg22392325 | chr1: 7049418 | CAMTA1.GENEBODY | SCLC-P | F | | |
| cg23746600 | chr6: 160403181 | IGF2R.GENEBODY | SCLC-P | F | | |
| cg25019648 | chr17: 72353261 | BTBD17.GENEBODY | SCLC-P | R | chr17: 72352827- | Island |
| | | | | | 72353798 | |
| cg25444002 | chr13: 102520143 | FGF14.GENEBODY | SCLC-P | R | | |
| cg27121870 | chr11: 134257030 | B3GAT1.GENEBODY | SCLC-P | R | chr11: 134257428- | N_Shore |
| | | | | | 134257631 | |
| cg27319536 | chr5: 146260901 | PPP2R2B.UTR5 | SCLC-P | R | chr5: 146257346- | S_Shelf |
| | | | | | 146258575 | |
| cg27382459 | chr2: 145237379 | ZEB2.GENEBODY | SCLC-P | F | | |
| cg27639080 | chr12: 49960363 | MCRS1.GENEBODY | SCLC-P | R | chr12: 49961630- | N_Shore |
| | | | | | 49962219 | |
| cg00762029 | chr4: 185317438 | IRF2.GENEBODY | SCLC-P | R | | |
| cg01217406 | chr15: 23013480 | NIPA2.GENEBODY | SCLC-P | R | | |
| cg01220513 | chrX: 19902129 | SH3KBP1.GENEBODY | SCLC-P | F | chrX: 19905027- | N_Shelf |
| | | | | | 19906557 | |
| cg01620165 | chr12: 113530915 | DTX1.GENEBODY | SCLC-P | R | chr12: 113530914- | Island |
| | | | | | 113531476 | |
| cg01708377 | chr6: 1686746 | GMDS.GENEBODY | SCLC-P | R | | |
| cg02291620 | chr1: 233365140 | PCNXL2.GENEBODY | SCLC-P | F | | |
| cg02394186 | chr1: 6088550 | KCNAB2.UTR5 | SCLC-P | F | chr1: 6086244-6086494 | S_Shelf |
| cg03389720 | chr16: 8780048 | ABAT.UTR5 | SCLC-P | R | | |
| cg04021887 | chr2: 10566989 | HPCAL1.UTR3 | SCLC-P | R | chr2: 10566889- | Island |
| | | | | | 10567148 | |
| cg05058976 | chr16: 3637956 | BTBD12.GENEBODY | SCLC-P | R | chr16: 3639092-3639306 | N_Shore |
| cg05157251 | chr18: 57566460 | PMAIP1.TSS1500 | SCLC-P | R | chr18: 57566866- | N_Shore |
| | | | | | 57567723 | |
| cg05407550 | chrX: 154250784 | F8.EXON1 | SCLC-P | R | | |
| cg05621843 | chr9: 97806397 | C9orf3.GENEBODY | SCLC-P | F | chr9: 97807450- | N_Shore |
| | | | | | 97807694 | |
| cg07038822 | chr11: 125830798 | CDON.UTR3 | SCLC-P | F | | |
| cg07507418 | chr6: 42372284 | TRERF1.UTR5 | SCLC-P | R | | |
| cg07716663 | chr19: 42636625 | POU2F2.UTR5 | SCLC-P | F | chr19: 42637277- | N_Shore |
| | | | | | 42637684 | |
| cg07768372 | chr5: 14509008 | TRIO.UTR3 | SCLC-P | F | | |
| cg07849009 | chr7: 105663410 | FLJ23834.GENEBODY | SCLC-P | F | | |
| cg07960154 | chr17: 37760198 | NEUROD2.UTR3 | SCLC-P | F | chr17: 37761691- | N_Shore |
| | | | | | 37763551 | |
| cg08438392 | chr2: 27708644 | IFT172.GENEBODY | SCLC-P | R | chr2: 27712554- | N_Shelf |
| | | | | | 27712842 | |
| cg09016768 | chr11: 118982033 | C2CD2L.GENEBODY | SCLC-P | F | chr11: 118977878- | S_Shelf |
| | | | | | 118978785 | |
| cg09640202 | chr2: 131101440 | IMP4.GENEBODY | SCLC-P | R | chr2: 131099469- | S_Shore |
| | | | | | 131100863 | |
| cg10090761 | chr19: 1468466 | APC2.GENEBODY | SCLC-P | F | chr19: 1465206-1471241 | Island |
| cg11650039 | chrX: 15525325 | BMX.UTR5 | SCLC-P | R | | |
| cg12110887 | chr19: 11096925 | SMARCA4.GENEBODY | SCLC-P | R | chr19: 11098395- | N_Shore |
| | | | | | 11098609 | |
| cg12184624 | chr10: 105239164 | CALHM3.TSS200 | SCLC-P | F | | |
| cg12426293 | chr18: 8612540 | RAB12.GENEBODY | SCLC-P | F | chr18: 8608763-8610370 | S_Shelf |
| cg12858166 | chr6: 33033176 | HLA-DPA1.UTR3 | SCLC-P | R | | |
| cg13027851 | chr2: 102433793 | MAP4K4.GENEBODY | SCLC-P | F | | |
| cg13403121 | chr7: 148963719 | ZNF783.GENEBODY | SCLC-P | R | chr7: 148958858- | S_Shelf |
| | | | | | 148959891 | |
| cg15076824 | chr1: 159685185 | CRP.TSS1500 | SCLC-P | F | | |
| cg15795819 | chr8: 39328433 | ADAM3A.GENEBODY | SCLC-P | R | | |
| cg16175713 | chr6: 46617172 | CYP39A1.GENEBODY | SCLC-P | F | chr6: 46620541- | N_Shelf |
| | | | | | 46621189 | |
| cg16430279 | chr1: 1266504 | TAS1R3.TSS1500 | SCLC-P | F | chr1: 1267085-1267286 | N_Shore |
| cg18457491 | chr10: 104829517 | CNNM2.GENEBODY | SCLC-P | R | | |
| cg18586851 | chr20: 31332353 | COMMD7.TSS1500 | SCLC-P | R | chr20: 31330957- | S_Shore |
| | | | | | 31331410 | |
| cg19237600 | chr7: 99564474 | AZGP1.UTR3 | SCLC-P | R | | |
| cg19305488 | chr19: 1468359 | APC2.GENEBODY | SCLC-P | F | chr19: 1465206-1471241 | Island |
| cg19861842 | chr8: 22855745 | RHOBTB2.GENEBODY | SCLC-P | R | chr8: 22856800- | N_Shore |
| | | | | | 22857728 | |
| cg20058937 | chr7: 5336543 | SLC29A4.GENEBODY | SCLC-P | R | chr7: 5336513-5336894 | Island |
| cg20668952 | chr15: 75082885 | CSK.UTR5 | SCLC-P | F | | |
| cg20927312 | chr3: 14443473 | SLC6A6.TSS1500 | SCLC-P | R | chr3: 14443629- | N_Shore |
| | | | | | 14444577 | |
| cg21702431 | chr17: 19617084 | SLC47A2.GENEBODY | SCLC-P | R | chr17: 19617083- | Island |
| | | | | | 19617424 | |
| cg23750365 | chr6: 33043072 | HLA-DPB1.TSS1500 | SCLC-P | F | | |
| cg24532476 | chr12: 133424566 | CHFR.GENEBODY | SCLC-P | F | | |
| cg24686845 | chr12: 94698752 | PLXNC1.GENEBODY | SCLC-P | F | | |
| cg25052933 | chr6: 33052888 | HLA-DPB1.GENEBODY | SCLC-P | F | | |
| cg25087423 | chr11: 118754535 | CXCR5.TSS200 | SCLC-P | R | | |
| cg25255988 | chr11: 75428927 | MOGAT2.TSS200 | SCLC-P | R | | |
| cg25901444 | chr7: 5336713 | SLC29A4.GENEBODY | SCLC-P | R | chr7: 5336513-5336894 | Island |
| cg26074140 | chr1: 44401309 | ARTN.TSS1500 | SCLC-P | F | chr1: 44401757- | N_Shore |
| | | | | | 44402423 | |
| cg26300785 | chr10: 81075492 | ZMIZ1.UTR3 | SCLC-P | F | | |
| cg26385743 | chr2: 131101463 | IMP4.GENEBODY | SCLC-P | R | chr2: 131099469- | S_Shore |
| | | | | | 131100863 | |
| cg26455386 | chr17: 16999983 | MPRIP.GENEBODY | SCLC-P | R | | |
| cg26576576 | chr1: 214627705 | PTPN14.GENEBODY | SCLC-P | R | | |
| cg27134386 | chr4: 46392485 | GABRA2.TSS1500 | SCLC-P | R | chr4: 46391903- | Island |
| | | | | | 46392572 | |
| cg27407935 | chr17: 17723235 | SREBF1.GENEBODY | SCLC-P | F | chr17: 17726855- | N_Shelf |
| | | | | | 17727282 | |
| cg00457932 | chr15: 28145216 | OCA2.GENEBODY | SCLC-P | F | chr15: 28147925- | N_Shelf |
| | | | | | 28148465 | |
| cg03402235 | chr15: 42749336 | ZFP106.EXON1 | SCLC-P | F | | |
| cg04245374 | chr1: 168150977 | TIPRL.GENEBODY | SCLC-P | F | chr1: 168147963- | S_Shelf |
| | | | | | 168148637 | |
| cg05571310 | chr17: 72350354 | KIF19.GENEBODY | SCLC-P | R | chr17: 72350346- | Island |
| | | | | | 72350711 | |
| cg06253072 | chr6: 33679850 | C6orf125.TSS1500 | SCLC-P | F | chr6: 33679158- | S_Shore |
| | | | | | 33679701 | |
| cg07209550 | chr5: 79070077 | CMYA5.GENEBODY | SCLC-P | R | | |
| cg09888186 | chr1: 183782195 | RGL1.GENEBODY | SCLC-P | R | | |
| cg10294240 | chr14: 96686415 | BDKRB2.UTR5 | SCLC-P | R | | |
| cg12194594 | chr13: 113426250 | ATP11A.GENEBODY | SCLC-P | R | chr13: 113424898- | S_Shore |
| | | | | | 113425105 | |
| cg13432241 | chr4: 154389285 | KIAA0922.GENEBODY | SCLC-P | F | chr4: 154386834- | S_Shore |
| | | | | | 154388183 | |
| cg17890764 | chr3: 52864816 | ITIH4.TSS200 | SCLC-P | R | | |
| cg18491744 | chr8: 41580834 | ANK1.GENEBODY | SCLC-P | F | chr8: 41583135- | N_Shelf |
| | | | | | 41583524 | |
| cg20069765 | chr6: 33036464 | HLA-DPA1.GENEBODY | SCLC-P | R | | |
| cg21670451 | chr5: 139781334 | ANKHD1.TSS200 | SCLC-P | F | chr5: 139780307- | Island |
| | | | | | 139782108 | |
| cg23272088 | chr11: 2504813 | KCNQ1.GENEBODY | SCLC-P | R | | |
| cg25227866 | chr17: 71489530 | SDK2.GENEBODY | SCLC-P | R | | |
| cg00093829 | chr10: 6247101 | PFKFB3.GENEBODY | SCLC-P | F | chr10: 6242975-6246457 | S_Shore |
| cg01027750 | chr19: 42636639 | POU2F2.TSS200 | SCLC-P | F | chr19: 42637277- | N_Shore |
| | | | | | 42637684 | |
| cg02782292 | chr4: 144433846 | SMARCA5.TSS1500 | SCLC-P | F | chr4: 144434249- | N_Shore |
| | | | | | 144435343 | |
| cg03040622 | chr7: 5536937 | MIR589.TSS1500 | SCLC-P | F | chr7: 5539957-5541567 | N_Shelf |
| cg03101058 | chr17: 71258936 | CPSF4L.TSS1500 | SCLC-P | R | | |
| cg03499581 | chr15: 78384868 | SH2D7.TSS200 | SCLC-P | R | | |
| cg04398759 | chr1: 203101066 | ADORA1.GENEBODY | SCLC-P | R | chr1: 203097233- | S_Shelf |
| | | | | | 203097496 | |
| cg04502517 | chr20: 62564156 | DNAJC5.UTR3 | SCLC-P | R | chr20: 62562545- | S_Shore |
| | | | | | 62562862 | |
| cg04908961 | chr13: 92003278 | MIR17HG.GENEBODY | SCLC-P | F | chr13: 91999541- | S_Shore |
| | | | | | 92001441 | |
| cg05030172 | chr19: 2078510 | MOBKL2A.GENEBODY | SCLC-P | F | chr19: 2078162-2078511 | Island |
| cg05137443 | chr6: 33094069 | HLA-DPB2.GENEBODY | SCLC-P | F | | |
| cg05299486 | chr17: 40609380 | ATP6VOA1.TSS1500 | SCLC-P | R | chr17: 40610746- | N_Shore |
| | | | | | 40611032 | |
| cg05451388 | chr3: 183817678 | HTR3E.TSS1500 | SCLC-P | F | | |
| cg05887270 | chr1: 26129627 | SEPN1.GENEBODY | SCLC-P | R | | |
| cg07097026 | chr6: 44257942 | TCTE1.UTR5 | SCLC-P | F | chr6: 44253897- | S_Shelf |
| | | | | | 44254150 | |
| cg07151747 | chr2: 158977473 | UPP2.GENEBODY | SCLC-P | F | | |
| cg07992044 | chr11: 68096049 | LRP5.GENEBODY | SCLC-P | F | | |
| cg08402572 | chr2: 97510201 | ANKRD23.TSS1500 | SCLC-P | F | | |
| cg10097316 | chr12: 7966784 | SLC2A14.UTR3 | SCLC-P | R | | |
| cg11356398 | chr10: 133783182 | BNIP3.GENEBODY | SCLC-P | F | | |
| cg11891522 | chr5: 61605247 | KIF2A.GENEBODY | SCLC-P | F | chr5: 61601617- | S_Shelf |
| | | | | | 61602930 | |
| cg12131414 | chr17: 72352968 | BTBD17.GENEBODY | SCLC-P | F | chr17: 72352827- | Island |
| | | | | | 72353798 | |
| cg12524428 | chr5: 146268595 | PPP2R2B.UTR5 | SCLC-P | F | | |
| cg12674490 | chr6: 31587470 | BAT2.TSS1500 | SCLC-P | R | chr6: 31587779- | N_Shore |
| | | | | | 31589024 | |
| cg12738981 | chr1: 155828357 | SYT11.TSS1500 | SCLC-P | R | chr1: 155829109- | N_Shore |
| | | | | | 155829431 | |
| cg13187009 | chr20: 55965497 | RBM38.TSS1500 | SCLC-P | R | chr20: 55966212- | N_Shore |
| | | | | | 55966964 | |
| cg13277337 | chr2: 240265810 | HDAC4.GENEBODY | SCLC-P | R | | |
| cg13594542 | chr9: 138850547 | UBAC1.GENEBODY | SCLC-P | R | chr9: 138852639- | N_Shelf |
| | | | | | 138853439 | |
| cg13925678 | chr19: 3522946 | FZR1.UTR5 | SCLC-P | F | | |
| cg14095761 | chr9: 35713238 | TLN1.GENEBODY | SCLC-P | F | | |
| cg14168316 | chr9: 127642782 | GOLGA1.UTR3 | SCLC-P | R | | |
| cg14173476 | chr17: 19190476 | EPN2.UTR5 | SCLC-P | F | | |
| cg15031661 | chr1: 240256603 | FMN2.EXON1 | SCLC-P | F | chr1: 240254959- | Island |
| | | | | | 240257063 | |
| cg15844109 | chrX: 78003205 | LPAR4.EXON1 | SCLC-P | F | | |
| cg16054907 | chr22: 50706065 | MAPK11.GENEBODY | SCLC-P | R | chr22: 50705406- | Island |
| | | | | | 50706553 | |
| cg16518986 | chr7: 157511420 | PTPRN2.GENEBODY | SCLC-P | F | | |
| cg17297045 | chr12: 125581343 | AACS.GENEBODY | SCLC-P | R | chr12: 125578348- | S_Shelf |
| | | | | | 125578588 | |
| cg17547928 | chr4: 737930 | PCGF3.GENEBODY | SCLC-P | F | chr4: 738424-741517 | N_Shore |
| cg17846127 | chr15: 89940505 | LOC254559.GENEBODY | SCLC-P | R | chr15: 89942592- | N_Shelf |
| | | | | | 89943853 | |
| cg18183642 | chr1: 44497694 | SLC6A9.TSS1500 | SCLC-P | F | chr1: 44495629- | S_Shore |
| | | | | | 44497112 | |
| cg18315680 | chr1: 154833117 | KCNN3.GENEBODY | SCLC-P | R | | |
| cg18927185 | chr6: 33091634 | HLA-DPB2.GENEBODY | SCLC-P | F | | |
| cg19062108 | chr12: 57630834 | NDUFA4L2.GENEBODY | SCLC-P | R | chr12: 57631932- | N_Shore |
| | | | | | 57634578 | |
| cg19482842 | chr2: 16790314 | FAM49A.UTR5 | SCLC-P | F | | |
| cg19710814 | chr6: 33091596 | HLA-DPB2.GENEBODY | SCLC-P | R | | |
| cg19995654 | chr12: 66628777 | IRAK3.GENEBODY | SCLC-P | R | | |
| cg20574732 | chr7: 158277801 | PTPRN2.GENEBODY | SCLC-P | F | chr7: 158277603- | Island |
| | | | | | 158278027 | |
| cg20643991 | chr5: 121690136 | SNCAIP.UTR5 | SCLC-P | F | | |
| cg21542666 | chr1: 248005715 | OR11L1.TSS1500 | SCLC-P | R | | |
| cg21570811 | chr4: 3362574 | RGS12.GENEBODY | SCLC-P | F | chr4: 3365194-3365415 | N_Shelf |
| cg21751684 | chr15: 89920887 | LOC254559.TSS1500 | SCLC-P | R | chr15: 89920793- | Island |
| | | | | | 89922768 | |
| cg21830221 | chr1: 42334284 | HIVEP3.UTR5 | SCLC-P | F | | |
| cg22646210 | chr11: 72380726 | PDE2A.TSS1500 | SCLC-P | F | | |
| cg23105863 | chr14: 101011318 | BEGAIN.GENEBODY | SCLC-P | F | chr14: 101012840- | N_Shore |
| | | | | | 101013117 | |
| cg23331433 | chr6: 33039606 | HLA-DPA1.GENEBODY | SCLC-P | F | | |
| cg23502492 | chr17: 42394379 | RUNDC3A.GENEBODY | SCLC-P | R | chr17: 42392323- | S_Shore |
| | | | | | 42393079 | |
| cg24940706 | chr19: 1648682 | TCF3.GENEBODY | SCLC-P | F | chr19: 1646117-1646713 | S_Shore |
| cg25057035 | chr6: 33053889 | HLA-DPB1.GENEBODY | SCLC-P | R | | |
| cg25516633 | chr8: 101315846 | RNF19A.TSS1500 | SCLC-P | F | | |
| cg26013412 | chr1: 247094486 | AHCTF1.GENEBODY | SCLC-P | R | chr1: 247094485- | Island |
| | | | | | 247095667 | |
| cg26164712 | chr11: 118754565 | CXCR5.UTR5 | SCLC-P | F | | |
| cg26283963 | chr7: 29330752 | CHN2.GENEBODY | SCLC-P | F | | |
| cg26603629 | chr7: 2752217 | AMZ1.GENEBODY | SCLC-P | F | | |
| cg27184903 | chr15: 29285727 | APBA2.UTR5 | SCLC-P | F | | |
| cg27191457 | chr15: 69105133 | ANP32A.GENEBODY | SCLC-P | F | | |
| cg27244524 | chr3: 38494523 | LOC100128640.GENEBODY | SCLC-P | F | chr3: 38495088- | N_Shore |
| | | | | | 38497151 | |
| cg27661104 | chr2: 232645033 | PDE6D.GENEBODY | SCLC-P | F | chr2: 232645313- | N_Shore |
| | | | | | 232646505 | |
| cg02982614 | chr11: 89391479 | FOLH1B.TSS1500 | SCLC-P | R | | |
| cg12348588 | chr17: 72352897 | BTBD17.GENEBODY | SCLC-P | R | chr17: 72352827- | Island |
| | | | | | 72353798 | |
| cg13993358 | chr5: 176304275 | UNC5A.GENEBODY | SCLC-P | R | chr5: 176304965- | N_Shore |
| | | | | | 176305166 | |
| cg19567891 | chr15: 89921083 | LOC254559.TSS200 | SCLC-P | F | chr15: 89920793- | Island |
| | | | | | 89922768 | |
| cg20371142 | chr20: 17295221 | PCSK2.GENEBODY | SCLC-P | F | chr20: 17296125- | N_Shore |
| | | | | | 17296531 | |
| cg20745419 | chr1: 151696417 | C1orf230.GENEBODY | SCLC-P | F | chr1: 151693991- | S_Shelf |
| | | | | | 151694282 | |
| cg24953213 | chr5: 172140 | PLEKHG4B.GENEBODY | SCLC-P | R | chr5: 169578-169798 | S_Shelf |
| cg00088251 | chr1: 1449754 | ATAD3A.UTR5 | SCLC-P | R | chr1: 1446878-1448205 | S_Shore |
| cg00481644 | chr15: 89921158 | LOC254559.TSS200 | SCLC-P | F | chr15: 89920793- | Island |
| | | | | | 89922768 | |
| cg00547530 | chr16: 3559687 | CLUAP1.GENEBODY | SCLC-P | F | | |
| cg00863271 | chr17: 76798776 | USP36.GENEBODY | SCLC-P | F | chr17: 76798154- | S_Shore |
| | | | | | 76798461 | |
| cg01011403 | chr4: 56500362 | NMU.GENEBODY | SCLC-P | F | chr4: 56501912- | N_Shore |
| | | | | | 56502523 | |
| cg01476047 | chr10: 73974007 | ASCC1.UTR5 | SCLC-P | F | chr10: 73975671- | N_Shore |
| | | | | | 73976115 | |
| cg01477971 | chr2: 103405951 | TMEM182.GENEBODY | SCLC-P | R | | |
| cg01663696 | chr12: 7942817 | NANOG.GENEBODY | SCLC-P | F | | |
| cg01833923 | chr17: 47286719 | GNGT2.EXON1 | SCLC-P | R | | |
| cg02140508 | chr12: 4659038 | RAD51AP1.GENEBODY | SCLC-P | F | | |
| cg02478836 | chr22: 47510037 | TBC1D22A.GENEBODY | SCLC-P | F | chr22: 47513202- | N_Shelf |
| | | | | | 47513585 | |
| cg03366884 | chr17: 8844506 | PIK3R5.UTR5 | SCLC-P | R | | |
| cg03368665 | chr11: 89819442 | UBTFL1.EXON1 | SCLC-P | R | | |
| cg03450107 | chr1: 41980863 | HIVEP3.GENEBODY | SCLC-P | R | chr1: 41981788- | N_Shore |
| | | | | | 41982043 | |
| cg04625873 | chr11: 118754530 | CXCR5.TSS200 | SCLC-P | F | | |
| cg05244974 | chr17: 8798734 | PIK3R5.GENEBODY | SCLC-P | R | | |
| cg05680240 | chr6: 31765195 | LSM2.UTR3 | SCLC-P | R | chr6: 31763240- | S_Shore |
| | | | | | 31763905 | |
| cg05766510 | chr7: 157454062 | PTPRN2.GENEBODY | SCLC-P | R | chr7: 157453774- | Island |
| | | | | | 157454208 | |
| cg05845757 | chr1: 36039885 | TFAP2E.GENEBODY | SCLC-P | F | chr1: 36038927- | Island |
| | | | | | 36040051 | |
| cg05913474 | chr8: 144815204 | FAM83H.UTR5 | SCLC-P | R | chr8: 144815203- | Island |
| | | | | | 144816518 | |
| cg05926586 | chr15: 74592786 | CCDC33.GENEBODY | SCLC-P | R | | |
| cg06788504 | chr6: 32974517 | HLA-DOA.UTR3 | SCLC-P | F | chr6: 32975684- | N_Shore |
| | | | | | 32975926 | |
| cg06860422 | chr16: 788079 | NARFL.GENEBODY | SCLC-P | F | chr16: 787070-787327 | S_Shore |
| cg07159802 | chr1: 247921568 | OR1C1.EXON1 | SCLC-P | F | | |
| cg07344019 | chr15: 69337685 | MIR548H4.GENEBODY | SCLC-P | F | | |
| cg07470396 | chr19: 54697539 | TSEN34.UTR3 | SCLC-P | F | chr19: 54693034- | S_Shelf |
| | | | | | 54695497 | |
| cg08192337 | chr12: 124982002 | NCOR2.UTR5 | SCLC-P | F | | |
| cg08210506 | chr14: 81456824 | TSHR.GENEBODY | SCLC-P | F | | |
| cg08797864 | chr3: 185531258 | IGF2BP2.GENEBODY | SCLC-P | R | | |
| cg08822746 | chr22: 31321152 | C22orf27.GENEBODY | SCLC-P | R | chr22: 31318228- | S_Shelf |
| | | | | | 31318489 | |
| cg08856378 | chr17: 560590 | VPS53.GENEBODY | SCLC-P | R | | |
| cg09274040 | chr10: 134994455 | KNDC1.GENEBODY | SCLC-P | R | chr10: 134996318- | N_Shore |
| | | | | | 134996548 | |
| cg09506037 | chr2: 134180560 | NCKAP5.GENEBODY | SCLC-P | F | | |
| cg09900440 | chr6: 33033312 | HLA-DPA1.UTR3 | SCLC-P | R | | |
| cg09910067 | chr7: 157357758 | PTPRN2.GENEBODY | SCLC-P | R | chr7: 157361512- | N_Shelf |
| | | | | | 157361805 | |
| cg10572969 | chr11: 13690188 | FAR1.TSS200 | SCLC-P | F | chr11: 13689588- | Island |
| | | | | | 13690724 | |
| cg11156873 | chr5: 1491579 | LPCAT1.GENEBODY | SCLC-P | R | chr5: 1489799-1490071 | S_Shore |
| cg11514462 | chr11: 121001293 | TECTA.GENEBODY | SCLC-P | F | chr11: 120998386- | S_Shelf |
| | | | | | 120998886 | |
| cg12192566 | chr5: 140011377 | CD14.UTR3 | SCLC-P | F | chr5: 140011482- | N_Shore |
| | | | | | 140012739 | |
| cg13337413 | chr17: 10728324 | PIRT.UTR3 | SCLC-P | F | | |
| cg13607138 | chr20: 33539306 | GSS.GENEBODY | SCLC-P | R | chr20: 33543082- | N_Shelf |
| | | | | | 33543846 | |
| cg14150071 | chr1: 33545982 | ADC.TSS1500 | SCLC-P | F | chr1: 33546524- | N_Shore |
| | | | | | 33547243 | |
| cg14286660 | chr7: 1050071 | C7orf50.GENEBODY | SCLC-P | F | chr7: 1048921-1049124 | S_Shore |
| cg14826031 | chr11: 75376361 | MAP6.GENEBODY | SCLC-P | F | chr11: 75378416- | N_Shelf |
| | | | | | 75379882 | |
| cg15005255 | chr19: 18368728 | KIAA1683.GENEBODY | SCLC-P | F | chr19: 18368482- | Island |
| | | | | | 18368911 | |
| cg15344504 | chr3: 31677078 | STT3B.GENEBODY | SCLC-P | F | | |
| cg17045790 | chr12: 6128269 | VWF.GENEBODY | SCLC-P | F | | |
| cg17771737 | chr19: 20150505 | ZNF682.TSS1500 | SCLC-P | R | chr19: 20149855- | S_Shore |
| | | | | | 20150175 | |
| cg18554789 | chr7: 139432178 | HIPK2.GENEBODY | SCLC-P | F | | |
| cg18559249 | chr17: 37760173 | NEUROD2.UTR3 | SCLC-P | F | chr17: 37761691- | N_Shore |
| | | | | | 37763551 | |
| cg19402335 | chr1: 159410002 | OR10J1.EXON1 | SCLC-P | R | | |
| cg20484653 | chr1: 204955033 | NFASC.GENEBODY | SCLC-P | F | chr1: 204950982- | S_Shelf |
| | | | | | 204951210 | |
| cg21045306 | chr12: 57637925 | STAC3.GENEBODY | SCLC-P | R | chr12: 57636557- | S_Shore |
| | | | | | 57637673 | |
| cg21378690 | chrX: 71694534 | HDAC8.UTR3 | SCLC-P | F | | |
| cg21745287 | chr12: 123464511 | ARL6IP4.TSS1500 | SCLC-P | R | chr12: 123464820- | N_Shore |
| | | | | | 123465972 | |
| cg22931292 | chr11: 17765568 | KCNC1.GENEBODY | SCLC-P | R | | |
| cg24627621 | chr6: 42109176 | C6orf132.GENEBODY | SCLC-P | R | chr6: 42109904- | N_Shore |
| | | | | | 42110361 | |
| cg24931632 | chr1: 47899667 | MGC12982.GENEBODY | SCLC-P | F | chr1: 47899661- | Island |
| | | | | | 47900385 | |
| cg25199552 | chr1: 23350054 | KDM1A.GENEBODY | SCLC-P | R | chr1: 23345814- | S_Shelf |
| | | | | | 23346677 | |
| cg25647583 | chr15: 91427184 | FES.TSS1500 | SCLC-P | F | chr15: 91427540- | N_Shore |
| | | | | | 91427905 | |
| cg25716623 | chr15: 89921845 | LOC254559.GENEBODY | SCLC-P | F | chr15: 89920793- | Island |
| | | | | | 89922768 | |
| cg26009944 | chr1: 52210504 | OSBPL9.UTR5 | SCLC-P | F | | |
| cg26355228 | chr2: 3260869 | TSSC1.GENEBODY | SCLC-P | F | | |
| cg26406292 | chr7: 75292248 | HIP1.GENEBODY | SCLC-P | F | | |
| cg27113326 | chr7: 157454068 | PTPRN2.GENEBODY | SCLC-P | R | chr7: 157453774- | Island |
| | | | | | 157454208 | |
| cg00108992 | chr16: 12192325 | SNX29.GENEBODY | SCLC-P | R | | |
| cg00168967 | chr8: 62505560 | ASPH.GENEBODY | SCLC-P | R | | |
| cg00256675 | chr4: 802962 | CPLX1.GENEBODY | SCLC-P | R | chr4: 798538-798984 | S_Shelf |
| cg00572387 | chr20: 1472914 | SIRPB2.TSS1500 | SCLC-P | R | | |
| cg00582579 | chr8: 135533786 | ZFAT.GENEBODY | SCLC-P | R | | |
| cg00629585 | chr9: 135853992 | GFI1B.TSS200 | SCLC-P | R | | |
| cg00869989 | chr19: 2774197 | SGTA.UTR5 | SCLC-P | F | chr19: 2774196-2774550 | Island |
| cg00957480 | chr17: 10728755 | PIRT.GENEBODY | SCLC-P | R | | |
| cg01120761 | chr12: 7903170 | CLEC4C.TSS1500 | SCLC-P | R | | |
| cg01953456 | chr3: 183817976 | HTR3E.UTR5 | SCLC-P | R | | |
| cg02264238 | chr3: 51426377 | MANF.GENEBODY | SCLC-P | R | chr3: 51428412- | N_Shelf |
| | | | | | 51429625 | |
| cg02308914 | chr3: 129290549 | PLXND1.GENEBODY | SCLC-P | R | | |
| cg02590486 | chr12: 499376 | KDM5A.TSS1500 | SCLC-P | R | chr12: 498098-498920 | S_Shore |
| cg02882755 | chr6: 34100963 | GRM4.EXON1 | SCLC-P | R | | |
| cg02915544 | chr11: 89392829 | FOLH1B.UTR5 | SCLC-P | R | | |
| cg03231860 | chr11: 128893940 | RICS.EXON1 | SCLC-P | F | | |
| cg03243669 | chr7: 1931776 | MAD1L1.GENEBODY | SCLC-P | F | | |
| cg03465847 | chrX: 40594937 | MED14.TSS200 | SCLC-P | F | chrX: 40594515- | Island |
| | | | | | 40595457 | |
| cg03766174 | chr16: 81547907 | CMIP.GENEBODY | SCLC-P | F | | |
| cg04428805 | chr17: 65239522 | HELZ.UTR5 | SCLC-P | R | chr17: 65240725- | N_Shore |
| | | | | | 65242498 | |
| cg04756594 | chr16: 24857601 | SLC5A11.EXON1 | SCLC-P | R | | |
| cg05647891 | chr4: 153346037 | FBXW7.UTR5 | SCLC-P | F | | |
| cg05794927 | chr1: 248059252 | OR2W3.EXON1 | SCLC-P | F | | |
| cg05938958 | chr1: 248224887 | OR2L3.EXON1 | SCLC-P | F | | |
| cg06222979 | chr18: 14543469 | POTEC.UTR5 | SCLC-P | F | | |
| cg06531741 | chr11: 113775450 | HTR3B.TSS200 | SCLC-P | F | | |
| cg06969933 | chr16: 88099740 | BANP.GENEBODY | SCLC-P | F | chr16: 88101473- | N_Shore |
| | | | | | 88101911 | |
| cg07474672 | chr6: 90142171 | ANKRD6.TSS1500 | SCLC-P | F | chr6: 90142751- | N_Shore |
| | | | | | 90143635 | |
| cg07825294 | chr19: 1415757 | DAZAP1.GENEBODY | SCLC-P | R | chr19: 1419751-1420405 | N_Shelf |
| cg07915189 | chr19: 7688621 | XAB2.GENEBODY | SCLC-P | R | chr19: 7688620-7688832 | Island |
| cg08468599 | chr1: 2182126 | SKI.GENEBODY | SCLC-P | F | | |
| cg08813944 | chr17: 71258589 | CPSF4L.TSS1500 | SCLC-P | F | | |
| cg08917933 | chr1: 19615303 | AKR7A3.TSS200 | SCLC-P | R | chr1: 19614998- | Island |
| | | | | | 19615660 | |
| cg08927102 | chr1: 1050110 | C1orf159.UTR5 | SCLC-P | F | chr1: 1050515-1050830 | N_Shore |
| cg09419176 | chr7: 157670705 | PTPRN2.GENEBODY | SCLC-P | R | chr7: 157669879- | S_Shore |
| | | | | | 157670095 | |
| cg09445097 | chr9: 130186736 | ZNF79.UTR5 | SCLC-P | R | chr9: 130186276- | Island |
| | | | | | 130187160 | |
| cg09867656 | chr1: 76550508 | ST6GALNAC3.GENEBODY | SCLC-P | R | | |
| cg09934892 | chr16: 3559551 | CLUAP1.GENEBODY | SCLC-P | F | | |
| cg10100117 | chr1: 179843111 | TOR1AIP2.UTR5 | SCLC-P | R | chr1: 179846879- | N_Shelf |
| | | | | | 179847106 | |
| cg10329874 | chr11: 66621442 | PC.GENEBODY | SCLC-P | R | chr11: 66623620- | N_Shelf |
| | | | | | 66626614 | |
| cg10409230 | chr17: 40318360 | KCNH4.GENEBODY | SCLC-P | F | | |
| cg10487770 | chr5: 57879443 | RAB3C.GENEBODY | SCLC-P | R | chr5: 57878725- | S_Shore |
| | | | | | 57879177 | |
| cg10719970 | chr6: 41529932 | FOXP4.UTR5 | SCLC-P | R | chr6: 41528266- | S_Shore |
| | | | | | 41528900 | |
| cg11375616 | chr21: 47287017 | PCBP3.GENEBODY | SCLC-P | R | chr21: 47287012- | Island |
| | | | | | 47287426 | |
| cg11397606 | chr1: 67151389 | SGIP1.GENEBODY | SCLC-P | R | | |
| cg11661493 | chr17: 74423978 | UBE2O.GENEBODY | SCLC-P | F | | |
| cg11866943 | chr3: 52529351 | STAB1.TSS200 | SCLC-P | F | | |
| cg12426652 | chr21: 37915056 | CLDN14.TSS200 | SCLC-P | F | | |
| cg12991341 | chr8: 109800287 | TMEM74.TSS1500 | SCLC-P | F | chr8: 109799429- | S_Shore |
| | | | | | 109799847 | |
| cg13372513 | chr7: 158177056 | PTPRN2.GENEBODY | SCLC-P | F | | |
| cg13534791 | chr9: 35042344 | C9orf131.EXON1 | SCLC-P | F | | |
| cg13995599 | chr13: 113426270 | ATP11A.GENEBODY | SCLC-P | R | chr13: 113424898- | S_Shore |
| | | | | | 113425105 | |
| cg14274019 | chr9: 134184504 | PPAPDC3.UTR3 | SCLC-P | R | chr9: 134183330- | S_Shore |
| | | | | | 134183613 | |
| cg14879948 | chr5: 1394576 | SLC6A3.UTR3 | SCLC-P | F | | |
| cg15147833 | chr1: 247921672 | OR1C1.EXON1 | SCLC-P | R | | |
| cg15617023 | chr13: 40113248 | LHFP.GENEBODY | SCLC-P | R | | |
| cg15871371 | chr3: 71274624 | FOXP1.UTR5 | SCLC-P | F | | |
| cg15871766 | chr12: 52365222 | ACVR1B.GENEBODY | SCLC-P | F | | |
| cg16748694 | chr10: 126754386 | CTBP2.UTR5 | SCLC-P | R | | |
| cg16895672 | chr10: 34470745 | PARD3.GENEBODY | SCLC-P | R | | |
| cg16983282 | chr17: 9871831 | GAS7.GENEBODY | SCLC-P | R | | |
| cg17013166 | chr22: 30961151 | GAL3ST1.TSS1500 | SCLC-P | R | | |
| cg17135722 | chr20: 17295815 | PCSK2.GENEBODY | SCLC-P | R | chr20: 17296125- | N_Shore |
| | | | | | 17296531 | |
| cg17179826 | chr1: 204946032 | NFASC.UTR3 | SCLC-P | R | | |
| cg17707347 | chr1: 234614967 | TARBP1.TSS200 | SCLC-P | F | chr1: 234613936- | Island |
| | | | | | 234614972 | |
| cg17810765 | chr2: 242127836 | ANO7.TSS200 | SCLC-P | R | | |
| cg17912283 | chr17: 72350487 | KIF19.GENEBODY | SCLC-P | F | chr17: 72350346- | Island |
| | | | | | 72350711 | |
| cg18502477 | chr11: 57946199 | OR9Q1.UTR5 | SCLC-P | F | | |
| cg18812946 | chrX: 153140985 | L1CAM.GENEBODY | SCLC-P | F | | |
| cg18861762 | chr2: 242127852 | ANO7.TSS200 | SCLC-P | F | | |
| cg19148404 | chr3: 14443444 | SLC6A6.TSS1500 | SCLC-P | F | chr3: 14443629- | N_Shore |
| | | | | | 14444577 | |
| cg19273683 | chr1: 21656047 | ECE1.GENEBODY | SCLC-P | R | | |
| cg19325793 | chr6: 33082165 | HLA-DPB2.GENEBODY | SCLC-P | F | chr6: 33084740- | N_Shelf |
| | | | | | 33084995 | |
| cg19393755 | chr17: 71258101 | CPSF4L.TSS200 | SCLC-P | F | | |
| cg19405329 | chr15: 49440632 | COPS2.GENEBODY | SCLC-P | F | | |
| cg19547192 | chr7: 158059976 | PTPRN2.GENEBODY | SCLC-P | R | chr7: 158060894- | N_Shore |
| | | | | | 158061100 | |
| cg19606462 | chr5: 7827964 | ADCY2.UTR3 | SCLC-P | R | | |
| cg20682981 | chr17: 1962627 | HIC1.UTR3 | SCLC-P | F | chr17: 1952919-1962328 | S_Shore |
| cg21029612 | chr15: 89921258 | LOC254559.TSS200 | SCLC-P | R | chr15: 89920793- | Island |
| | | | | | 89922768 | |
| cg21552242 | chr3: 157814326 | SHOX2.UTR3 | SCLC-P | R | chr3: 157815581- | N_Shore |
| | | | | | 157816095 | |
| cg21703606 | chr12: 54427884 | HOXC4.UTR5 | SCLC-P | R | chr12: 54427025- | Island |
| | | | | | 54428709 | |
| cg21900589 | chr8: 22866294 | RHOBTB2.GENEBODY | SCLC-P | F | chr8: 22864324- | S_Shore |
| | | | | | 22864588 | |
| cg22791545 | chr17: 1924319 | RTN4RL1.GENEBODY | SCLC-P | R | chr17: 1927051-1929408 | N_Shelf |
| cg22821554 | chr7: 99383155 | CYP3A4.TSS1500 | SCLC-P | F | | |
| cg23055499 | chr12: 51847363 | SLC4A8.GENEBODY | SCLC-P | F | | |
| cg23172995 | chr5: 150486801 | ANXA6.GENEBODY | SCLC-P | R | | |
| cg23714707 | chr17: 17418691 | PEMT.GENEBODY | SCLC-P | R | | |
| cg23833461 | chr17: 9929528 | GAS7.EXON1 | SCLC-P | R | | |
| cg24603972 | chr10: 61434211 | SLC16A9.GENEBODY | SCLC-P | F | | |
| cg25129480 | chr1: 185126704 | C1orf25.TSS1500 | SCLC-P | F | chr1: 185125850- | Island |
| | | | | | 185126705 | |
| cg25312122 | chr1: 160184732 | PEA15.UTR3 | SCLC-P | R | | |
| cg25425390 | chr12: 42763958 | PPHLN1.GENEBODY | SCLC-P | R | | |
| cg25985583 | chr7: 26404716 | SNX10.GENEBODY | SCLC-P | R | | |
| cg26306056 | chr6: 32972989 | HLA-DOA.UTR3 | SCLC-P | R | chr6: 32975684- | N_Shelf |
| | | | | | 32975926 | |
| cg26353877 | chr1: 159557886 | APCS.GENEBODY | SCLC-P | R | | |
| cg26558420 | chr20: 30331315 | TPX2.UTR5 | SCLC-P | R | chr20: 30326913- | S_Shelf |
| | | | | | 30327430 | |
| cg26577231 | chr19: 18368873 | KIAA1683.GENEBODY | SCLC-P | F | chr19: 18368482- | Island |
| | | | | | 18368911 | |
| cg26942930 | chr7: 147804524 | CNTNAP2.GENEBODY | SCLC-P | R | | |
| cg27150178 | chr19: 1456337 | APC2.GENEBODY | SCLC-P | F | chr19: 1456077-1456347 | Island |
| cg01173794 | chr19: 36131225 | ETV2.TSS1500 | SCLC-P | F | chr19: 36134153- | N_Shelf |
| | | | | | 36135781 | |
| cg24774182 | chr15: 22017473 | CXADRP2.TSS1500 | SCLC-P | F | | |
| cg27541374 | chr17: 58042493 | RNFT1.TSS1500 | SCLC-P | R | chr17: 58041846- | S_Shore |
| | | | | | 58042463 | |
| cg00401265 | chrX: 78003113 | LPAR4.TSS200 | SCLC-P | F | | |
| cg00435063 | chr7: 157454207 | PTPRN2.GENEBODY | SCLC-P | F | chr7: 157453774- | Island |
| | | | | | 157454208 | |
| cg00515756 | chr11: 61464876 | DAGLA.UTR5 | SCLC-P | R | | |
| cg00653387 | chr7: 137028535 | PTN.UTR5 | SCLC-P | F | | |
| cg00808053 | chr12: 6125746 | VWF.GENEBODY | SCLC-P | F | | |
| cg00808511 | chr16: 52495237 | TOX3.GENEBODY | SCLC-P | R | | |
| cg01132064 | chr11: 46382562 | DGKZ.GENEBODY | SCLC-P | F | | |
| cg01403030 | chr9: 19026479 | FAM154A.GENEBODY | SCLC-P | R | chr9: 19027850- | N_Shore |
| | | | | | 19028064 | |
| cg01507200 | chr1: 247877066 | OR6F1.TSS1500 | SCLC-P | R | | |
| cg01708636 | chr4: 166301085 | CPE.GENEBODY | SCLC-P | R | chr4: 166299975- | S_Shore |
| | | | | | 166300668 | |
| cg02077068 | chr16: 19872240 | GPRC5B.GENEBODY | SCLC-P | R | | |
| cg03081691 | chr1: 226188006 | C1orf55.TSS1500 | SCLC-P | F | chr1: 226186803- | S_Shore |
| | | | | | 226187336 | |
| cg03306486 | chr19: 1467952 | APC2.GENEBODY | SCLC-P | F | chr19: 1465206-1471241 | Island |
| cg04383012 | chr2: 231921692 | PSMD1.EXON1 | SCLC-P | R | chr2: 231921413- | Island |
| | | | | | 231921972 | |
| cg04655481 | chr9: 125796809 | GPR21.TSS200 | SCLC-P | F | | |
| cg04665588 | chr17: 14243776 | HS3ST3B1.GENEBODY | SCLC-P | F | | |
| cg04804648 | chr17: 72348295 | KIF19.GENEBODY | SCLC-P | F | chr17: 72347924- | Island |
| | | | | | 72348322 | |
| cg04809274 | chr6: 32977254 | HLA-DOA.EXON1 | SCLC-P | F | chr6: 32975684- | S_Shore |
| | | | | | 32975926 | |
| cg04962134 | chr11: 55653310 | SPRYD5.GENEBODY | SCLC-P | R | | |
| cg05221455 | chr11: 829616 | EFCAB4A.GENEBODY | SCLC-P | F | chr11: 829284-833558 | Island |
| cg05720937 | chr2: 98280362 | ACTR1B.EXON1 | SCLC-P | R | chr2: 98280063- | Island |
| | | | | | 98280871 | |
| cg06517181 | chr10: 134994252 | KNDC1.GENEBODY | SCLC-P | F | chr10: 134993121- | S_Shore |
| | | | | | 134993324 | |
| cg06572563 | chr19: 1132966 | SBNO2.GENEBODY | SCLC-P | R | | |
| cg07100542 | chr1: 17402079 | PADI2.GENEBODY | SCLC-P | R | | |
| cg07436694 | chr16: 21638444 | METTL9.GENEBODY | SCLC-P | R | | |
| cg07586799 | chr3: 195591722 | TNK2.GENEBODY | SCLC-P | R | chr3: 195594836- | N_Shelf |
| | | | | | 195595494 | |
| cg07870237 | chr5: 131348431 | ACSL6.TSS1500 | SCLC-P | F | chr5: 131346892- | S_Shore |
| | | | | | 131347776 | |
| cg08984900 | chr17: 34430281 | CCL4.TSS1500 | SCLC-P | R | | |
| cg09524686 | chr11: 829182 | EFCAB4A.GENEBODY | SCLC-P | F | chr11: 829284-833558 | N_Shore |
| cg09565226 | chr16: 84759648 | USP10.GENEBODY | SCLC-P | R | | |
| cg10256976 | chr11: 20078682 | NAV2.GENEBODY | SCLC-P | F | | |
| cg10327976 | chr15: 42843303 | HAUS2.GENEBODY | SCLC-P | R | chr15: 42840462- | S_Shelf |
| | | | | | 42841116 | |
| cg10341513 | chr22: 51041242 | MAPK8IP2.TSS1500 | SCLC-P | R | chr22: 51042293- | N_Shore |
| | | | | | 51043422 | |
| cg10521450 | chr10: 105439280 | SH3PXD2A.GENEBODY | SCLC-P | R | | |
| cg10959048 | chr3: 93694020 | PROS1.TSS1500 | SCLC-P | R | chr3: 93692554- | S_Shore |
| | | | | | 93692783 | |
| cg11049863 | chr17: 80832470 | TBCD.GENEBODY | SCLC-P | F | chr17: 80829533- | S_Shelf |
| | | | | | 80829871 | |
| cg11150074 | chr1: 200863559 | C1orf106.TSS1500 | SCLC-P | R | chr1: 200860076- | S_Shelf |
| | | | | | 200860576 | |
| cg11321144 | chr4: 151178020 | DCLK2.UTR3 | SCLC-P | F | chr4: 151177347- | S_Shore |
| | | | | | 151177559 | |
| cg11521965 | chr7: 137028604 | PTN.TSS200 | SCLC-P | F | | |
| cg11581706 | chr7: 82072141 | CACNA2D1.GENEBODY | SCLC-P | F | chr7: 82072021- | Island |
| | | | | | 82073520 | |
| cg12196847 | chr14: 81426577 | TSHR.GENEBODY | SCLC-P | R | | |
| cg12617146 | chr10: 97101836 | SORBS1.GENEBODY | SCLC-P | R | | |
| cg13617795 | chr7: 105137683 | PUS7.GENEBODY | SCLC-P | F | | |
| cg13698125 | chr9: 140391480 | PNPLA7.GENEBODY | SCLC-P | R | chr9: 140389453- | S_Shore |
| | | | | | 140389751 | |
| cg13921245 | chr6: 33053791 | HLA-DPB1.GENEBODY | SCLC-P | F | | |
| cg14022090 | chr11: 34642303 | EHF.TSS1500 | SCLC-P | F | | |
| cg14147151 | chr9: 138948000 | NACC2.UTR5 | SCLC-P | R | chr9: 138941470- | S_Shelf |
| | | | | | 138944021 | |
| cg14322298 | chr6: 10585683 | GCNT2.GENEBODY | SCLC-P | F | | |
| cg14402562 | chr11: 8945810 | C11orf16.GENEBODY | SCLC-P | R | | |
| cg15287806 | chr10: 237794 | ZMYND11.GENEBODY | SCLC-P | R | | |
| cg15332132 | chr20: 62183964 | C20orf195.TSS1500 | SCLC-P | R | chr20: 62185073- | N_Shore |
| | | | | | 62185572 | |
| cg15367000 | chr2: 239017149 | ESPNL.GENEBODY | SCLC-P | F | | |
| cg15463309 | chr8: 141598157 | EIF2C2.GENEBODY | SCLC-P | F | | |
| cg16182685 | chr2: 3260980 | TSSC1.GENEBODY | SCLC-P | F | | |
| cg16280667 | chr11: 118754593 | CXCR5.UTR5 | SCLC-P | F | | |
| cg16477417 | chr17: 73342168 | GRB2.GENEBODY | SCLC-P | F | | |
| cg16711332 | chr1: 6531727 | PLEKHG5.GENEBODY | SCLC-P | F | chr1: 6530536-6531814 | Island |
| cg16791781 | chr11: 986922 | AP2A2.GENEBODY | SCLC-P | R | | |
| cg17777731 | chr6: 32976131 | HLA-DOA.GENEBODY | SCLC-P | R | chr6: 32975684- | S_Shore |
| | | | | | 32975926 | |
| cg17947172 | chr1: 204966197 | NFASC.GENEBODY | SCLC-P | R | | |
| cg18702239 | chrX: 69663606 | DLG3.TSS1500 | SCLC-P | F | chrX: 69665102- | N_Shore |
| | | | | | 69665355 | |
| cg18715243 | chr8: 37658755 | GPR124.GENEBODY | SCLC-P | R | chr8: 37654048- | S_Shelf |
| | | | | | 37656100 | |
| cg18969798 | chr4: 151157003 | DCLK2.GENEBODY | SCLC-P | R | | |
| cg19185706 | chr10: 88475771 | LDB3.GENEBODY | SCLC-P | F | chr10: 88470905- | S_Shelf |
| | | | | | 88471934 | |
| cg19409156 | chr15: 48413040 | SLC24A5.TSS200 | SCLC-P | R | | |
| cg19427746 | chr17: 4889932 | CAMTA2.UTR5 | SCLC-P | R | chr17: 4890282-4890991 | N_Shore |
| cg20100408 | chr6: 33050888 | HLA-DPB1.GENEBODY | SCLC-P | F | chr6: 33048416- | S_Shelf |
| | | | | | 33048814 | |
| cg20541233 | chr21: 18938039 | CXADR.UTR3 | SCLC-P | R | | |
| cg20907136 | chr6: 33041221 | HLA-DPA1.GENEBODY | SCLC-P | F | | |
| cg20938170 | chr11: 75428910 | MOGAT2.TSS200 | SCLC-P | R | | |
| cg21748244 | chr5: 169713179 | LCP2.GENEBODY | SCLC-P | F | | |
| cg21776674 | chr1: 6160143 | KCNAB2.UTR3 | SCLC-P | F | | |
| cg22007216 | chr4: 155312410 | DCHS2.GENEBODY | SCLC-P | R | | |
| cg22381352 | chr1: 146742008 | CHD1L.GENEBODY | SCLC-P | R | | |
| cg22442557 | chr17: 79011352 | BAIAP2.GENEBODY | SCLC-P | R | chr17: 79008929- | S_Shore |
| | | | | | 79009762 | |
| cg22858170 | chr5: 39204386 | FYB.UTR5 | SCLC-P | R | | |
| cg23095615 | chr6: 146136563 | FBXO30.TSS1500 | SCLC-P | F | chr6: 146136325- | Island |
| | | | | | 146136564 | |
| cg23326197 | chr7: 99382370 | CYP3A4.TSS1500 | SCLC-P | F | | |
| cg23645261 | chr19: 30940563 | ZNF536.GENEBODY | SCLC-P | F | chr19: 30941853- | N_Shore |
| | | | | | 30942181 | |
| cg23676767 | chr1: 226918736 | ITPKB.GENEBODY | SCLC-P | R | | |
| cg23699947 | chr12: 27167077 | TM7SF3.EXON1 | SCLC-P | F | chr12: 27166996- | Island |
| | | | | | 27167464 | |
| cg23777946 | chr18: 24125409 | KCTD1.UTR5 | SCLC-P | R | chr18: 24126780- | N_Shore |
| | | | | | 24131138 | |
| cg23998119 | chr14: 77600217 | ZDHHC22.GENEBODY | SCLC-P | R | | |
| cg24214000 | chr22: 50282386 | ZBED4.UTR3 | SCLC-P | F | chr22: 50279568- | S_Shelf |
| | | | | | 50280376 | |
| cg24396358 | chr6: 32977400 | HLA-DOA.TSS200 | SCLC-P | F | chr6: 32975684- | S_Shore |
| | | | | | 32975926 | |
| cg24436022 | chr22: 45691661 | UPK3A.UTR3 | SCLC-P | F | | |
| cg24625894 | chr8: 145638202 | SLC39A4.GENEBODY | SCLC-P | R | chr8: 145637965- | Island |
| | | | | | 145639182 | |
| cg24678581 | chr5: 170882542 | FGF18.GENEBODY | SCLC-P | R | chr5: 170883600- | N_Shore |
| | | | | | 170883836 | |
| cg24938100 | chr6: 33091407 | HLA-DPB2.GENEBODY | SCLC-P | R | | |
| cg25693265 | chr7: 6517860 | KDELR2.GENEBODY | SCLC-P | R | | |
| cg26345142 | chr12: 109305794 | SVOP.UTR3 | SCLC-P | F | | |
| cg26490671 | chr7: 157670732 | PTPRN2.GENEBODY | SCLC-P | R | chr7: 157669879- | S_Shore |
| | | | | | 157670095 | |
| cg26692016 | chr12: 7817844 | APOBEC1.GENEBODY | SCLC-P | R | | |
| cg26871279 | chr11: 2423559 | TSSC4.UTR5 | SCLC-P | R | chr11: 2421040-2422474 | S_Shore |
| cg27504782 | chr7: 94284258 | SGCE.GENEBODY | SCLC-P | R | chr7: 94284858- | N_Shore |
| | | | | | 94286527 | |
| cg00731886 | chr4: 1363008 | KIAA1530.GENEBODY | SCLC-P | F | chr4: 1364146-1364349 | N_Shore |
| cg00896220 | chr17: 34639327 | CCL4L1.TSS1500 | SCLC-P | R | | |
| cg01443488 | chr1: 159557514 | APCS.TSS200 | SCLC-P | F | | |
| cg01924561 | chr1: 43416103 | SLC2A1.GENEBODY | SCLC-P | F | | |
| cg04810997 | chr7: 143140674 | TAS2R60.EXON1 | SCLC-P | R | | |
| cg05677694 | chr6: 33052797 | HLA-DPB1.GENEBODY | SCLC-P | F | chr6: 33048416- | S_Shelf |
| | | | | | 33048814 | |
| cg11070176 | chr11: 70489806 | SHANK2.GENEBODY | SCLC-P | F | | |
| cg11941526 | chrX: 38660299 | MID1IP1.TSS1500 | SCLC-P | F | chrX: 38662906- | N_Shelf |
| | | | | | 38664764 | |
| cg14517144 | chr10: 70098420 | HNRNPH3.GENEBODY | SCLC-P | F | | |
| cg14684709 | chr13: 100547249 | CLYBL.UTR3 | SCLC-P | F | chr13: 100547172- | Island |
| | | | | | 100547431 | |
| cg16537306 | chr17: 9850268 | GAS7.GENEBODY | SCLC-P | R | | |
| cg17753661 | chr1: 26441637 | PDIK1L.GENEBODY | SCLC-P | F | chr1: 26437319- | S_Shelf |
| | | | | | 26438917 | |
| cg23710594 | chr1: 33841330 | PHC2.TSS200 | SCLC-P | R | | |
| cg00935894 | chr8: 110705093 | GOLSYN.TSS1500 | SCLC-P | R | chr8: 110703409- | S_Shore |
| | | | | | 110703897 | |
| cg01083652 | chr15: 71629208 | THSD4.GENEBODY | SCLC-P | R | | |
| cg02075590 | chr4: 125607552 | ANKRD50.UTR5 | SCLC-P | R | | |
| cg02169973 | chr19: 50983986 | C19orf63.GENEBODY | SCLC-P | F | chr19: 50983853- | Island |
| | | | | | 50984075 | |
| cg02220617 | chr14: 102312533 | PPP2R5C.GENEBODY | SCLC-P | R | | |
| cg02555215 | chr12: 113497560 | DTX1.GENEBODY | SCLC-P | R | chr12: 113494389- | S_Shelf |
| | | | | | 113495534 | |
| cg02963493 | chr19: 11568915 | ELAVL3.GENEBODY | SCLC-P | R | chr19: 11565384- | S_Shelf |
| | | | | | 11565711 | |
| cg03252568 | chr17: 41727259 | MEOX1.GENEBODY | SCLC-P | F | chr17: 41723219- | S_Shelf |
| | | | | | 41723826 | |
| cg01862650 | chr5: 143619194 | KCTD16.GENEBODY | SCLC-P | R | | |
| cg01828813 | chr6: 33132705 | COL11A2.GENEBODY | SCLC-P | R | chr6: 33129291- | S_Shelf |
| | | | | | 33129718 | |
| cg19887293 | chr6: 33132693 | COL11A2.GENEBODY | SCLC-P | R | chr6: 33129291- | S_Shelf |
| | | | | | 33129718 | |
| cg25736198 | chr12: 72345225 | TPH2.GENEBODY | SCLC-P | R | | |
| cg01159294 | chr5: 143763289 | KCTD16.GENEBODY | SCLC-P | F | | |
| cg23009046 | chr11: 119600595 | PVRL1.TSS1500 | SCLC-P | R | chr11: 119598382- | S_Shore |
| | | | | | 119600157 | |
| cg26548251 | chr7: 147591462 | MIR548F3.TSS1500 | SCLC-P | F | | |
| cg00177243 | chr10: 18434068 | CACNB2.GENEBODY | SCLC-P | F | chr10: 18429125- | S_Shelf |
| | | | | | 18430234 | |
| cg03050127 | chr17: 59413660 | BCAS3.GENEBODY | SCLC-P | R | | |
| cg26357587 | chr11: 120110882 | POU2F3.TSS200 | SCLC-P | R | chr11: 120110497- | S_Shore |
| | | | | | 120110719 | |
| cg02965092 | chr12: 72347052 | TPH2.GENEBODY | SCLC-P | F | | |
| cg07961632 | chr1: 27668421 | SYTL1.TSS200 | SCLC-P | R | | |
| cg10380643 | chr1: 1356764 | LOC441869.TSS200 | SCLC-P | R | chr1: 1355159-1356000 | S_Shore |
| cg17790159 | chr6: 33133083 | COL11A2.GENEBODY | SCLC-P | R | chr6: 33129291- | S_Shelf |
| | | | | | 33129718 | |
| cg14154859 | chr1: 220702826 | MARK1.GENEBODY | SCLC-P | F | chr1: 220701507- | S_Shore |
| | | | | | 220702348 | |
| cg10120778 | chr7: 42018795 | GLI3.GENEBODY | SCLC-P | F | | |
| cg15700587 | chr7: 147065665 | MIR54814.GENEBODY | SCLC-P | R | | |
| cg00333034 | chr3: 183948058 | VWA5B2.TSS1500 | SCLC-I | F | chr3: 183947040- | Island |
| | | | | | 183948801 | |
| cg00789347 | chr16: 2039030 | SYNGR3.TSS1500 | SCLC-I | F | chr16: 2040034-2040628 | N_Shore |
| cg01507577 | chr7: 146484609 | CNTNAP2.GENEBODY | SCLC-I | R | | |
| cg02379560 | chr4: 40859280 | APBB2.GENEBODY | SCLC-I | F | chr4: 40858884- | S_Shore |
| | | | | | 40859162 | |
| cg04917391 | chr4: 40859259 | APBB2.GENEBODY | SCLC-I | F | chr4: 40858884- | S_Shore |
| | | | | | 40859162 | |
| cg05894734 | chr4: 40859249 | APBB2.GENEBODY | SCLC-I | F | chr4: 40858884- | S_Shore |
| | | | | | 40859162 | |
| cg06485940 | chr4: 40859293 | APBB2.GENEBODY | SCLC-I | F | chr4: 40858884- | S_Shore |
| | | | | | 40859162 | |
| cg07035001 | chr7: 146321053 | CNTNAP2.GENEBODY | SCLC-I | F | | |
| cg12455762 | chr4: 40859095 | APBB2.GENEBODY | SCLC-I | F | chr4: 40858884- | Island |
| | | | | | 40859162 | |
| cg14230238 | chr19: 6066871 | RFX2.UTR5 | SCLC-I | R | | |
| cg14248352 | chr7: 146820536 | CNTNAP2.GENEBODY | SCLC-I | R | | |
| cg14652038 | chr2: 222343519 | EPHA4.GENEBODY | SCLC-I | R | | |
| cg16364495 | chr4: 40859251 | APBB2.GENEBODY | SCLC-I | F | chr4: 40858884- | S_Shore |
| | | | | | 40859162 | |
| cg22519702 | chr5: 147204189 | SPINK1.UTR3 | SCLC-I | R | | |
| cg23735712 | chr4: 40858984 | APBB2.GENEBODY | SCLC-I | F | chr4: 40858884- | Island |
| | | | | | 40859162 | |
| cg23889772 | chr19: 10077324 | COL5A3.GENEBODY | SCLC-I | F | chr19: 10077031- | Island |
| | | | | | 10077444 | |
| cg25649655 | chr8: 145692504 | CYHR1.TSS1500 | SCLC-I | F | chr8: 145690605- | Island |
| | | | | | 145692505 | |
| cg26244738 | chr4: 40859344 | APBB2.GENEBODY | SCLC-I | R | chr4: 40858884- | S_Shore |
| | | | | | 40859162 | |
| cg26470314 | chr16: 2039411 | SYNGR3.TSS1500 | SCLC-I | R | chr16: 2040034-2040628 | N_Shore |
| cg01657995 | chr6: 31804883 | C6orf48.UTR5 | SCLC-I | F | chr6: 31802598- | S_Shelf |
| | | | | | 31802823 | |
| cg02269097 | chr12: 49487944 | DHH.GENEBODY | SCLC-I | R | chr12: 49487963- | N_Shore |
| | | | | | 49488202 | |
| cg03392565 | chr1: 212464932 | PPP2R5A.GENEBODY | SCLC-I | R | | |
| cg03957440 | chr1: 214209324 | PROX1.UTR3 | SCLC-I | R | | |
| cg09050775 | chr12: 46762708 | SLC38A2.GENEBODY | SCLC-I | R | chr12: 46765134- | N_Shelf |
| | | | | | 46766633 | |
| cg11526866 | chr1: 20813614 | CAMK2N1.TSS1500 | SCLC-I | F | chr1: 20810462- | S_Shore |
| | | | | | 20813511 | |
| cg13109300 | chr12: 49488587 | DHH.UTR5 | SCLC-I | F | chr12: 49487963- | S_Shore |
| | | | | | 49488202 | |
| cg16151451 | chr11: 849153 | TSPAN4.UTR5 | SCLC-I | F | chr11: 849087-850982 | Island |
| cg16405211 | chr7: 146897691 | CNTNAP2.GENEBODY | SCLC-I | R | | |
| cg17490790 | chr19: 10125043 | RDH8.GENEBODY | SCLC-I | R | chr19: 10120750- | S_Shelf |
| | | | | | 10121231 | |
| cg18804920 | chr7: 146572638 | CNTNAP2.GENEBODY | SCLC-I | R | | |
| cg19075717 | chr17: 48670940 | CACNA1G.GENEBODY | SCLC-I | R | | |
| cg24127874 | chr2: 239149859 | HES6.TSS1500 | SCLC-I | F | chr2: 239147592- | Island |
| | | | | | 239149900 | |
| cg04303901 | chr12: 97886312 | RMST.GENEBODY | SCLC-I | F | | |
| cg04695027 | chr11: 65793986 | CATSPER1.EXON1 | SCLC-I | R | chr11: 65789909- | S_Shelf |
| | | | | | 65790168 | |
| cg04865716 | chr19: 2290843 | LINGO3.GENEBODY | SCLC-I | R | chr19: 2289895-2291904 | Island |
| cg05582979 | chr4: 10686610 | CLNK.TSS1500 | SCLC-I | F | | |
| cg06093379 | chr17: 44896080 | WNT3.UTR5 | SCLC-I | R | chr17: 44895800- | Island |
| | | | | | 44897025 | |
| cg08384314 | chr6: 30711680 | IER3.UTR3 | SCLC-I | R | chr6: 30710307- | Island |
| | | | | | 30712440 | |
| cg09267188 | chr8: 97340225 | PTDSS1.GENEBODY | SCLC-I | R | | |
| cg12724192 | chr4: 82392157 | RASGEF1B.UTR5 | SCLC-I | R | chr4: 82392610- | N_Shore |
| | | | | | 82393426 | |
| cg13067221 | chr14: 23449582 | JUB.GENEBODY | SCLC-I | F | chr14: 23449886- | N_Shore |
| | | | | | 23452174 | |
| cg16406078 | chr20: 825634 | FAM110A.GENEBODY | SCLC-I | F | chr20: 825267-826276 | Island |
| cg17971747 | chr15: 45410236 | DUOXA1.GENEBODY | SCLC-I | F | chr15: 45408573- | S_Shore |
| | | | | | 45409528 | |
| cg22942704 | chr1: 20813574 | CAMK2N1.TSS1500 | SCLC-I | R | chr1: 20810462- | S_Shore |
| | | | | | 20813511 | |
| cg25151498 | chr3: 185815992 | ETV5.GENEBODY | SCLC-I | F | | |
| cg25564319 | chr3: 63558416 | SYNPR.GENEBODY | SCLC-I | F | | |
| cg27190967 | chr16: 2039391 | SYNGR3.TSS1500 | SCLC-I | F | chr16: 2040034-2040628 | N_Shore |
| cg01920182 | chr12: 23738340 | SOX5.TSS1500 | SCLC-I | R | | |
| cg03595580 | chr7: 97880848 | TECPR1.UTR5 | SCLC-I | F | chr7: 97881132- | N_Shore |
| | | | | | 97881906 | |
| cg04993130 | chr11: 726759 | EPS8L2.GENEBODY | SCLC-I | R | chr11: 725596-726870 | Island |
| cg06736134 | chr5: 147040725 | JAKMIP2.GENEBODY | SCLC-I | R | | |
| cg09248054 | chr1: 969257 | AGRN.GENEBODY | SCLC-I | F | chr1: 967966-970238 | Island |
| cg11565786 | chr5: 31944970 | PDZD2.GENEBODY | SCLC-I | R | | |
| cg16769791 | chr8: 85178030 | RALYL.UTR5 | SCLC-I | R | | |
| cg19741167 | chr2: 86263270 | POLR1A.GENEBODY | SCLC-I | R | | |
| cg25225073 | chr14: 90528983 | KCNK13.GENEBODY | SCLC-I | F | chr14: 90526696- | S_Shore |
| | | | | | 90528951 | |
| cg26683639 | chr6: 30653755 | KIAA1949.GENEBODY | SCLC-I | F | chr6: 30654392- | N_Shore |
| | | | | | 30654934 | |
| cg07788437 | chr19: 1799952 | ATP8B3.GENEBODY | SCLC-I | R | chr19: 1799934-1800248 | Island |
| cg11207515 | chr7: 146904205 | CNTNAP2.GENEBODY | SCLC-I | R | | |
| cg11540204 | chr1: 60391699 | CYP2J2.GENEBODY | SCLC-I | R | chr1: 60392272- | N_Shore |
| | | | | | 60392583 | |
| cg11799006 | chr5: 147101975 | JAKMIP2.UTR5 | SCLC-I | R | | |
| cg13369952 | chr5: 147024959 | JAKMIP2.GENEBODY | SCLC-I | F | | |
| cg14074581 | chr19: 36232218 | TMEM149.UTR5 | SCLC-I | F | chr19: 36231186- | Island |
| | | | | | 36232219 | |
| cg25183613 | chr3: 111717557 | TAGLN3.TSS200 | SCLC-I | F | | |
| cg00967013 | chr13: 24735754 | SPATA13.UTR5 | SCLC-I | F | chr13: 24734771- | S_Shore |
| | | | | | 24735538 | |
| cg02120463 | chr22: 39639003 | PDGFB.GENEBODY | SCLC-I | F | chr22: 39638219- | Island |
| | | | | | 39640966 | |
| cg02578321 | chr1: 247275872 | C1orf229.TSS200 | SCLC-I | R | chr1: 247274585- | S_Shore |
| | | | | | 247275757 | |
| cg03415518 | chr12: 97885729 | RMST.GENEBODY | SCLC-I | R | | |
| cg04513006 | chr16: 68270252 | ESRP2.TSS200 | SCLC-I | F | chr16: 68269010- | Island |
| | | | | | 68272143 | |
| cg05825085 | chr16: 86379872 | LOC732275.TSS1500 | SCLC-I | F | | |
| cg06185146 | chr3: 63428305 | SYNPR.GENEBODY | SCLC-I | F | | |
| cg08669018 | chr4: 82392280 | RASGEF1B.UTR5 | SCLC-I | R | chr4: 82392610- | N_Shore |
| | | | | | 82393426 | |
| cg09473576 | chr1: 20813647 | CAMK2N1.TSS1500 | SCLC-I | F | chr1: 20810462- | S_Shore |
| | | | | | 20813511 | |
| cg10083593 | chr11: 66049855 | CNIH2.GENEBODY | SCLC-I | R | chr11: 66049750- | Island |
| | | | | | 66050229 | |
| cg11408784 | chr17: 7166293 | CLDN7.TSS1500 | SCLC-I | F | chr17: 7164285-7166245 | S_Shore |
| cg11876912 | chr3: 63602009 | SYNPR.UTR3 | SCLC-I | F | | |
| cg13250388 | chr22: 39641078 | PDGFB.TSS200 | SCLC-I | R | chr22: 39638219- | S_Shore |
| | | | | | 39640966 | |
| cg14615553 | chr5: 147093466 | JAKMIP2.UTR5 | SCLC-I | F | | |
| cg15149374 | chr3: 19576826 | KCNH8.UTR3 | SCLC-I | R | | |
| cg15225810 | chr19: 750790 | C19orf21.TSS1500 | SCLC-I | F | chr19: 746300-747161 | S_Shelf |
| cg17075538 | chr19: 15306892 | NOTCH3.GENEBODY | SCLC-I | R | chr19: 15306243- | Island |
| | | | | | 15307111 | |
| cg17410295 | chr12: 109729809 | FOXN4.GENEBODY | SCLC-I | F | chr12: 109729572- | Island |
| | | | | | 109729826 | |
| cg17475643 | chr8: 10261972 | MSRA.GENEBODY | SCLC-I | F | chr8: 10261900- | Island |
| | | | | | 10262222 | |
| cg19464419 | chr6: 94064439 | EPHA7.GENEBODY | SCLC-I | R | | |
| cg20391895 | chr4: 10686394 | CLNK.TSS200 | SCLC-I | F | | |
| cg24238564 | chr11: 65779343 | CST6.TSS200 | SCLC-I | F | chr11: 65779311- | Island |
| | | | | | 65779767 | |
| cg26939195 | chr6: 32077020 | TNXB.UTR5 | SCLC-I | F | | |
| cg00026290 | chr4: 10686554 | CLNK.TSS200 | SCLC-I | F | | |
| cg02785561 | chr8: 10262221 | MSRA.GENEBODY | SCLC-I | F | chr8: 10261900- | Island |
| | | | | | 10262222 | |
| cg04221461 | chr1: 243877024 | AKT3.GENEBODY | SCLC-I | R | | |
| cg05082805 | chr7: 146489513 | CNTNAP2.GENEBODY | SCLC-I | F | | |
| cg09433131 | chr8: 73480179 | KCNB2.GENEBODY | SCLC-I | F | | |
| cg11652360 | chr6: 33160120 | COL11A2.UTR5 | SCLC-I | R | chr6: 33159991- | Island |
| | | | | | 33160263 | |
| cg19311889 | chr12: 57483793 | NAB2.GENEBODY | SCLC-I | R | chr12: 57480797- | S_Shore |
| | | | | | 57483116 | |
| cg00835193 | chr19: 2291780 | LINGO3.UTR5 | SCLC-I | R | chr19: 2289895-2291904 | Island |
| cg01708411 | chr12: 57631839 | NDUFA4L2.UTR5 | SCLC-I | R | chr12: 57631932- | N_Shore |
| | | | | | 57634578 | |
| cg02958126 | chr11: 62370246 | EML3.GENEBODY | SCLC-I | F | chr11: 62368454- | Island |
| | | | | | 62370491 | |
| cg02989255 | chr6: 32063774 | TNXB.GENEBODY | SCLC-I | F | chr6: 32063533- | Island |
| | | | | | 32065044 | |
| cg03525956 | chr3: 174720287 | NAALADL2.GENEBODY | SCLC-I | F | | |
| cg06191495 | chr2: 234264163 | DGKD.GENEBODY | SCLC-I | R | | |
| cg11809958 | chr12: 32654929 | FGD4.TSS200 | SCLC-I | R | | |
| cg11845241 | chr7: 36960377 | MIR1200.TSS1500 | SCLC-I | R | | |
| cg13179469 | chr13: 107185675 | EFNB2.GENEBODY | SCLC-I | F | chr13: 107186468- | N_Shore |
| | | | | | 107189024 | |
| cg18349863 | chr6: 29912713 | HLA-A.GENEBODY | SCLC-I | F | chr6: 29910202- | S_Shore |
| | | | | | 29911367 | |
| cg22138393 | chr8: 97340270 | PTDSS1.GENEBODY | SCLC-I | F | | |
| cg26726230 | chr17: 79225857 | SLC38A10.GENEBODY | SCLC-I | F | | |
| cg26961166 | chr8: 73687160 | KCNB2.GENEBODY | SCLC-I | F | | |
| cg26999775 | chr14: 105347803 | KIAA0284.GENEBODY | SCLC-I | R | chr14: 105350177- | N_Shelf |
| | | | | | 105350925 | |
| cg00433917 | chr19: 7915517 | EVI5L.GENEBODY | SCLC-I | F | chr19: 7913751-7913968 | S_Shore |
| cg01422274 | chr11: 849088 | TSPAN4.UTR5 | SCLC-I | R | chr11: 849087-850982 | Island |
| cg02245020 | chr7: 143580147 | FAM115A.UTR5 | SCLC-I | R | chr7: 143582125- | N_Shore |
| | | | | | 143582610 | |
| cg04545963 | chr14: 35872393 | NFKBIA.GENEBODY | SCLC-I | F | chr14: 35873047- | N_Shore |
| | | | | | 35873990 | |
| cg07384913 | chr3: 63637118 | SNTN.TSS1500 | SCLC-I | F | | |
| cg07706352 | chr19: 10077245 | COL5A3.GENEBODY | SCLC-I | R | chr19: 10077031- | Island |
| | | | | | 10077444 | |
| cg08279008 | chr2: 86263224 | POLR1A.GENEBODY | SCLC-I | F | | |
| cg10144863 | chr8: 53235234 | ST18.UTR5 | SCLC-I | R | | |
| cg11743827 | chr3: 185080905 | MAP3K13.TSS200 | SCLC-I | R | | |
| cg13720581 | chr6: 32077017 | TNXB.UTR5 | SCLC-I | F | | |
| cg15148573 | chr7: 103498566 | RELN.GENEBODY | SCLC-I | F | | |
| cg16078836 | chr3: 185081374 | MAP3K13.UTR5 | SCLC-I | R | | |
| cg16226037 | chr3: 175338261 | NAALADL2.GENEBODY | SCLC-I | R | | |
| cg16715129 | chr11: 10477821 | AMPD3.EXON1 | SCLC-I | F | | |
| cg19418951 | chr3: 185080816 | MAP3K13.TSS200 | SCLC-I | R | | |
| cg24519146 | chr6: 33160318 | COL11A2.TSS200 | SCLC-I | F | chr6: 33160945- | N_Shore |
| | | | | | 33161337 | |
| cg25264265 | chr3: 185080997 | MAP3K13.EXON1 | SCLC-I | R | | |
| cg25479682 | chr3: 185081403 | MAP3K13.UTR5 | SCLC-I | R | | |
| cg26334804 | chr19: 41724208 | AXL.TSS1500 | SCLC-I | R | | |
| cg27287254 | chr6: 33160067 | COL11A2.UTR5 | SCLC-I | F | chr6: 33159991- | Island |
| | | | | | 33160263 | |
| cg27633342 | chr15: 52588279 | MYO5C.TSS1500 | SCLC-I | R | chr15: 52587353- | S_Shore |
| | | | | | 52588172 | |
| cg00037056 | chr4: 956607 | DGKQ.GENEBODY | SCLC-I | F | chr4: 960505-960836 | N_Shelf |
| cg01484141 | chr17: 1624157 | WDR81.GENEBODY | SCLC-I | R | chr17: 1627745-1629897 | N_Shelf |
| cg01679081 | chr5: 60139131 | ELOVL7.UTR5 | SCLC-I | F | chr5: 60139359- | N_Shore |
| | | | | | 60140155 | |
| cg01886125 | chr2: 110054030 | SH3RF3.GENEBODY | SCLC-I | F | | |
| cg03508107 | chr17: 47654357 | NXPH3.GENEBODY | SCLC-I | F | chr17: 47653211- | Island |
| | | | | | 47654369 | |
| cg03676636 | chr4: 99064102 | C4orf37.GENEBODY | SCLC-I | R | chr4: 99064602- | N_Shore |
| | | | | | 99064805 | |
| cg03850035 | chr2: 222367110 | EPHA4.GENEBODY | SCLC-I | R | | |
| cg04083466 | chr6: 25753732 | SLC17A4.TSS1500 | SCLC-I | F | | |
| cg05396397 | chr1: 11908164 | NPPA.TSS1500 | SCLC-I | R | | |
| cg07107420 | chr7: 8010894 | GLCCI1.GENEBODY | SCLC-I | F | chr7: 8008097-8009495 | S_Shore |
| cg10242279 | chr4: 10666415 | CLNK.GENEBODY | SCLC-I | R | | |
| cg12286569 | chr19: 18722620 | TMEM59L.TSS1500 | SCLC-I | R | chr19: 18722358- | Island |
| | | | | | 18724001 | |
| cg13298116 | chr11: 62369859 | EML3.UTR3 | SCLC-I | R | chr11: 62368454- | Island |
| | | | | | 62370491 | |
| cg14848284 | chr2: 73143293 | EMX1.TSS1500 | SCLC-I | R | chr2: 73143055- | Island |
| | | | | | 73148260 | |
| cg16686429 | chr19: 46284983 | DMPK.GENEBODY | SCLC-I | F | chr19: 46282544- | S_Shore |
| | | | | | 46283127 | |
| cg18164281 | chr20: 2797721 | LOC100288797.UTR3 | SCLC-I | R | chr20: 2801639-2802135 | N_Shelf |
| cg18776945 | chr5: 147024842 | JAKMIP2.GENEBODY | SCLC-I | R | | |
| cg19250002 | chr22: 20460068 | RIMBP3.EXON1 | SCLC-I | F | chr22: 20459267- | Island |
| | | | | | 20461473 | |
| cg19268434 | chr6: 32076967 | TNXB.UTR5 | SCLC-I | F | | |
| cg22076422 | chr3: 63399168 | SYNPR.GENEBODY | SCLC-I | R | | |
| cg22168512 | chr18: 74695224 | MBP.GENEBODY | SCLC-I | F | chr18: 74691262- | S_Shelf |
| | | | | | 74691466 | |
| cg26248155 | chr17: 7165849 | CLDN7.TSS200 | SCLC-I | R | chr17: 7164285-7166245 | Island |
| cg26574680 | chr1: 235811860 | GNG4.UTR5 | SCLC-I | F | chr1: 235811841- | Island |
| | | | | | 235814066 | |
| cg26627129 | chr19: 49552470 | CGB8.TSS200 | SCLC-I | F | | |
| cg00102996 | chr9: 92220617 | GADD45G.GENEBODY | SCLC-I | R | chr9: 92219771- | Island |
| | | | | | 92221411 | |
| cg01589998 | chr12: 109729478 | FOXN4.GENEBODY | SCLC-I | R | chr12: 109729572- | N_Shore |
| | | | | | 109729826 | |
| cg01994275 | chr7: 8480586 | NXPH1.GENEBODY | SCLC-I | F | chr7: 8481974-8482762 | N_Shore |
| cg04013592 | chr3: 63263076 | SYNPR.TSS1500 | SCLC-I | F | chr3: 63263989- | N_Shore |
| | | | | | 63264205 | |
| cg05848863 | chr7: 16794078 | TSPAN13.GENEBODY | SCLC-I | R | chr7: 16793188- | Island |
| | | | | | 16794100 | |
| cg09649438 | chr10: 81904160 | PLAC9.GENEBODY | SCLC-I | R | | |
| cg13223402 | chr2: 47797133 | KCNK12.EXON1 | SCLC-I | R | chr2: 47796923- | Island |
| | | | | | 47799166 | |
| cg13827782 | chr9: 93377568 | DIRAS2.UTR5 | SCLC-I | R | | |
| cg13978352 | chr6: 30908706 | DPCR1.TSS200 | SCLC-I | R | | |
| cg14750475 | chr19: 50139654 | RRAS.GENEBODY | SCLC-I | F | chr19: 50143223- | N_Shelf |
| | | | | | 50143975 | |
| cg15954675 | chr3: 63542363 | SYNPR.GENEBODY | SCLC-I | R | | |
| cg19195419 | chr19: 1799935 | ATP8B3.GENEBODY | SCLC-I | R | chr19: 1799934-1800248 | Island |
| cg20083442 | chr8: 73600575 | KCNB2.GENEBODY | SCLC-I | R | | |
| cg20353496 | chr20: 825268 | FAM110A.TSS200 | SCLC-I | R | chr20: 825267-826276 | Island |
| cg21574349 | chr3: 185080951 | MAP3K13.TSS200 | SCLC-I | F | | |
| cg23422361 | chr10: 135143617 | CALY.UTR5 | SCLC-I | F | chr10: 135143373- | S_Shore |
| | | | | | 135143580 | |
| cg23463608 | chr19: 2607757 | GNG7.UTR5 | SCLC-I | F | chr19: 2607725-2607967 | Island |
| cg27233151 | chr4: 82057467 | PRKG2.GENEBODY | SCLC-I | R | | |
| cg02365303 | chr5: 147052498 | JAKMIP2.UTR5 | SCLC-I | F | | |
| cg08694699 | chr16: 68270282 | ESRP2.TSS200 | SCLC-I | F | chr16: 68269010- | Island |
| | | | | | 68272143 | |
| cg20668718 | chr5: 146991927 | JAKMIP2.GENEBODY | SCLC-I | F | | |
| cg20818262 | chr8: 73480145 | KCNB2.GENEBODY | SCLC-I | R | | |
| cg22459924 | chr19: 2607850 | GNG7.UTR5 | SCLC-I | F | chr19: 2607725-2607967 | Island |
| cg11610754 | chr19: 18722595 | TMEM59L.TSS1500 | SCLC-I | R | chr19: 18722358- | Island |
| | | | | | 18724001 | |
| cg12053155 | chr13: 107186476 | EFNB2.GENEBODY | SCLC-I | R | chr13: 107186468- | Island |
| | | | | | 107189024 | |
| cg12232903 | chr4: 10686413 | CLNK.TSS200 | SCLC-I | F | | |
| cg14995475 | chr19: 6534774 | TNFSF9.GENEBODY | SCLC-I | R | chr19: 6534759-6535079 | Island |
| cg16661000 | chr15: 89437710 | HAPLN3.UTR5 | SCLC-I | F | chr15: 89438176- | N_Shore |
| | | | | | 89438850 | |
| cg17208427 | chr16: 68679556 | CDH3.GENEBODY | SCLC-I | R | chr16: 68678686- | Island |
| | | | | | 68680149 | |
| cg18721026 | chr3: 183948313 | VWA5B2.TSS200 | SCLC-I | F | chr3: 183947040- | Island |
| | | | | | 183948801 | |
| cg00090619 | chr6: 33160286 | COL11A2.TSS200 | SCLC-I | F | chr6: 33160945- | N_Shore |
| | | | | | 33161337 | |
| cg00106565 | chr5: 146784171 | DPYSL3.GENEBODY | SCLC-I | F | | |
| cg00303541 | chr3: 51741280 | GRM2.UTR5 | SCLC-I | R | chr3: 51740740- | Island |
| | | | | | 51741413 | |
| cg01177388 | chr6: 32812704 | PSMB8.UTR5 | SCLC-I | F | chr6: 32811494- | S_Shore |
| | | | | | 32811839 | |
| cg01294327 | chr19: 2291373 | LINGO3.GENEBODY | SCLC-I | R | chr19: 2289895-2291904 | Island |
| cg02688752 | chr5: 76115309 | F2RL1.GENEBODY | SCLC-I | R | chr5: 76115510- | N_Shore |
| | | | | | 76116089 | |
| cg02969706 | chr2: 6072283 | LOC150622.TSS1500 | SCLC-I | R | | |
| cg03225210 | chr7: 143579951 | FAM115A.UTR5 | SCLC-I | R | chr7: 143582125- | N_Shelf |
| | | | | | 143582610 | |
| cg04458159 | chr6: 29856538 | HLA-H.GENEBODY | SCLC-I | F | chr6: 29855295- | Island |
| | | | | | 29856565 | |
| cg04872123 | chr6: 32049699 | TNXB.GENEBODY | SCLC-I | R | chr6: 32046815- | S_Shelf |
| | | | | | 32047094 | |
| cg05057976 | chr11: 65779667 | CST6.EXON1 | SCLC-I | F | chr11: 65779311- | Island |
| | | | | | 65779767 | |
| cg06321045 | chr6: 31088343 | CDSN.TSS200 | SCLC-I | F | | |
| cg06529264 | chr13: 54887288 | MIR1297.TSS1500 | SCLC-I | F | | |
| cg07435445 | chr14: 103396379 | AMN.GENEBODY | SCLC-I | R | chr14: 103394397- | Island |
| | | | | | 103397070 | |
| cg08551725 | chr7: 30029776 | SCRN1.TSS1500 | SCLC-I | F | chr7: 30028518- | Island |
| | | | | | 30029822 | |
| cg11606261 | chr12: 53775336 | SP1.UTR5 | SCLC-I | R | chr12: 53774336- | S_Shore |
| | | | | | 53774603 | |
| cg11794801 | chr8: 10262020 | MSRA.GENEBODY | SCLC-I | R | chr8: 10261900- | Island |
| | | | | | 10262222 | |
| cg11941546 | chr17: 7165842 | CLDN7.TSS200 | SCLC-I | R | chr17: 7164285-7166245 | Island |
| cg13083674 | chr19: 55953372 | SHISA7.GENEBODY | SCLC-I | R | chr19: 55953371- | Island |
| | | | | | 55954641 | |
| cg13470920 | chr19: 6772831 | VAV1.EXON1 | SCLC-I | F | | |
| cg13723983 | chr9: 132383362 | C9orf50.TSS1500 | SCLC-I | F | chr9: 132382432- | S_Shore |
| | | | | | 132383004 | |
| cg13851989 | chr9: 14720244 | CER1.GENEBODY | SCLC-I | R | | |
| cg13984492 | chr9: 104031694 | LPPR1.GENEBODY | SCLC-I | R | | |
| cg15705054 | chr1: 154927709 | PBXIP1.UTR5 | SCLC-I | R | | |
| cg17930034 | chr14: 65016602 | C14orf50.TSS200 | SCLC-I | R | chr14: 65016516- | Island |
| | | | | | 65016909 | |
| cg20114890 | chr6: 29856430 | HLA-H.GENEBODY | SCLC-I | R | chr6: 29855295- | Island |
| | | | | | 29856565 | |
| cg21476000 | chr12: 54430684 | HOXC4.UTR5 | SCLC-I | F | chr12: 54427025- | S_Shore |
| | | | | | 54428709 | |
| cg21552300 | chr2: 175629224 | CHRNA1.TSS200 | SCLC-I | F | | |
| cg23292266 | chr19: 11285128 | KANK2.GENEBODY | SCLC-I | R | chr19: 11285122- | Island |
| | | | | | 11285332 | |
| cg23517605 | chr6: 3228365 | TUBB2B.TSS1500 | SCLC-I | F | chr6: 3227025-3229688 | Island |
| cg24735489 | chr6: 31088352 | CDSN.TSS200 | SCLC-I | F | | |
| cg25036095 | chr4: 82392410 | RASGEF1B.UTR5 | SCLC-I | F | chr4: 82392610- | N_Shore |
| | | | | | 82393426 | |
| cg25580825 | chr19: 10116808 | COL5A3.GENEBODY | SCLC-I | F | chr19: 10120750- | N_Shelf |
| | | | | | 10121231 | |
| cg25661884 | chr19: 10124267 | RDH8.EXON1 | SCLC-I | R | chr19: 10120750- | S_Shelf |
| | | | | | 10121231 | |
| cg25751198 | chr12: 109729573 | FOXN4.GENEBODY | SCLC-I | R | chr12: 109729572- | Island |
| | | | | | 109729826 | |
| cg00378510 | chr19: 2291020 | LINGO3.GENEBODY | SCLC-I | F | chr19: 2289895-2291904 | Island |
| cg17259886 | chr5: 147040667 | JAKMIP2.GENEBODY | SCLC-I | F | | |
| cg00961418 | chr11: 8616172 | STK33.TSS1500 | SCLC-I | F | chr11: 8615397-8615889 | S_Shore |
| cg01218350 | chr2: 210517441 | MAP2.UTR5 | SCLC-I | R | | |
| cg01974715 | chr19: 41724129 | AXL.TSS1500 | SCLC-I | F | | |
| cg02374996 | chr9: 92220771 | GADD45G.GENEBODY | SCLC-I | F | chr9: 92219771- | Island |
| | | | | | 92221411 | |
| cg03250393 | chr19: 41735107 | AXL.GENEBODY | SCLC-I | F | chr19: 41732006- | S_Shelf |
| | | | | | 41732556 | |
| cg03436967 | chr7: 147207052 | MIR54814.GENEBODY | SCLC-I | F | | |
| cg03549571 | chr12: 49488198 | DHH.EXON1 | SCLC-I | R | chr12: 49487963- | Island |
| | | | | | 49488202 | |
| cg03832506 | chrX: 53286187 | IQSEC2.GENEBODY | SCLC-I | R | chrX: 53283829- | S_Shelf |
| | | | | | 53284038 | |
| cg04328477 | chr16: 68270258 | ESRP2.TSS200 | SCLC-I | F | chr16: 68269010- | Island |
| | | | | | 68272143 | |
| cg05171933 | chr5: 147205668 | SPINK1.GENEBODY | SCLC-I | F | | |
| cg06193043 | chr1: 11908199 | NPPA.TSS1500 | SCLC-I | F | | |
| cg06205574 | chr12: 23858674 | SOX5.GENEBODY | SCLC-I | F | | |
| cg06671964 | chr6: 32052105 | TNXB.GENEBODY | SCLC-I | F | chr6: 32055067- | N_Shelf |
| | | | | | 32055601 | |
| cg06723863 | chr16: 68270129 | ESRP2.EXON1 | SCLC-I | F | chr16: 68269010- | Island |
| | | | | | 68272143 | |
| cg07896312 | chr8: 22409062 | SORBS3.TSS200 | SCLC-I | R | chr8: 22408672- | Island |
| | | | | | 22409635 | |
| cg08416875 | chr6: 119669226 | MAN1A1.GENEBODY | SCLC-I | R | chr6: 119669589- | N_Shore |
| | | | | | 119671424 | |
| cg08575922 | chr12: 49488082 | DHH.EXON1 | SCLC-I | R | chr12: 49487963- | Island |
| | | | | | 49488202 | |
| cg09807148 | chr17: 34122069 | MMP28.GENEBODY | SCLC-I | F | chr17: 34122024- | Island |
| | | | | | 34122801 | |
| cg09990600 | chr7: 1596103 | TMEM184A.TSS200 | SCLC-I | R | | |
| cg10030250 | chr16: 55540152 | MMP2.UTR3 | SCLC-I | F | chr16: 55542920- | N_Shelf |
| | | | | | 55543274 | |
| cg10131879 | chr7: 8011594 | GLCCI1.GENEBODY | SCLC-I | F | chr7: 8008097-8009495 | S_Shelf |
| cg10688790 | chr7: 147591961 | MIR548F3.TSS1500 | SCLC-I | R | | |
| cg11384924 | chr6: 33160224 | COL11A2.UTR5 | SCLC-I | F | chr6: 33159991- | Island |
| | | | | | 33160263 | |
| cg11664251 | chr2: 149634581 | KIF5C.GENEBODY | SCLC-I | R | chr2: 149632682- | S_Shore |
| | | | | | 149633882 | |
| cg11989343 | chr13: 53287193 | LECT1.GENEBODY | SCLC-I | R | | |
| cg12444861 | chr6: 144386275 | PLAGL1.TSS1500 | SCLC-I | F | chr6: 144384894- | S_Shore |
| | | | | | 144385888 | |
| cg12626076 | chr15: 92399195 | SLCO3A1.GENEBODY | SCLC-I | F | chr15: 92396013- | S_Shore |
| | | | | | 92397682 | |
| cg13586420 | chr6: 33160262 | COL11A2.TSS200 | SCLC-I | F | chr6: 33159991- | Island |
| | | | | | 33160263 | |
| cg13969788 | chr9: 35689475 | TPM2.GENEBODY | SCLC-I | F | chr9: 35689610- | N_Shore |
| | | | | | 35691101 | |
| cg14011070 | chr1: 235811811 | GNG4.UTR5 | SCLC-I | R | chr1: 235811841- | N_Shore |
| | | | | | 235814066 | |
| cg14627974 | chr7: 147188028 | MIR54814.GENEBODY | SCLC-I | F | | |
| cg15558616 | chr3: 173630182 | NLGN1.GENEBODY | SCLC-I | R | | |
| cg15678504 | chr8: 10262159 | MSRA.GENEBODY | SCLC-I | R | chr8: 10261900- | Island |
| | | | | | 10262222 | |
| cg15712559 | chr20: 58180616 | PHACTR3.GENEBODY | SCLC-I | F | chr20: 58179808- | Island |
| | | | | | 58180787 | |
| cg15982299 | chr6: 31097078 | PSORS1C1.UTR5 | SCLC-I | F | | |
| cg16014168 | chr12: 109729677 | FOXN4.GENEBODY | SCLC-I | F | chr12: 109729572- | Island |
| | | | | | 109729826 | |
| cg16911349 | chr19: 10406935 | ICAM5.GENEBODY | SCLC-I | R | chr19: 10406934- | Island |
| | | | | | 10407342 | |
| cg18216587 | chr7: 146019420 | CNTNAP2.GENEBODY | SCLC-I | F | | |
| cg18516816 | chr19: 30165591 | PLEKHF1.UTR3 | SCLC-I | F | chr19: 30164768- | S_Shore |
| | | | | | 30165556 | |
| cg19047707 | chr2: 233351525 | ECEL1.UTR5 | SCLC-I | R | chr2: 233350278- | Island |
| | | | | | 233352974 | |
| cg20010135 | chr16: 30996822 | HSD3B7.UTR5 | SCLC-I | F | | |
| cg20330126 | chr22: 36125053 | APOL5.UTR3 | SCLC-I | F | | |
| cg21446725 | chr12: 49740996 | DNAJC22.TSS200 | SCLC-I | R | chr12: 49738680- | S_Shore |
| | | | | | 49740841 | |
| cg22857356 | chr19: 8398590 | KANK3.GENEBODY | SCLC-I | R | chr19: 8397958-8400461 | Island |
| cg23097961 | chr19: 49055412 | SULT2B1.TSS200 | SCLC-I | F | | |
| cg23344395 | chr3: 192634002 | C3orf59.GENEBODY | SCLC-I | R | chr3: 192634309- | N_Shore |
| | | | | | 192635967 | |
| cg23623622 | chr6: 30139880 | TRIM15.GENEBODY | SCLC-I | F | chr6: 30139718- | Island |
| | | | | | 30140263 | |
| cg23764900 | chr11: 8616102 | STK33.TSS1500 | SCLC-I | R | chr11: 8615397-8615889 | S_Shore |
| cg23883945 | chr7: 36749772 | AOAH.GENEBODY | SCLC-I | F | | |
| cg23937993 | chr11: 10477847 | AMPD3.EXON1 | SCLC-I | F | | |
| cg24021846 | chr7: 103556529 | RELN.GENEBODY | SCLC-I | F | | |
| cg24952408 | chr6: 29696264 | LOC285830.GENEBODY | SCLC-I | F | | |
| cg25934371 | chr3: 63427866 | SYNPR.GENEBODY | SCLC-I | F | | |
| cg27023555 | chr2: 233351735 | ECEL1.UTR5 | SCLC-I | R | chr2: 233350278- | Island |
| | | | | | 233352974 | |
| cg27108362 | chr12: 49488554 | DHH.UTR5 | SCLC-I | F | chr12: 49487963- | S_Shore |
| | | | | | 49488202 | |
| cg27181765 | chr6: 33160250 | COL11A2.TSS200 | SCLC-I | F | chr6: 33159991- | Island |
| | | | | | 33160263 | |
| cg01635061 | chr6: 33160234 | COL11A2.UTR5 | SCLC-I | F | chr6: 33159991- | Island |
| | | | | | 33160263 | |
| cg02582213 | chr16: 89441654 | ANKRD11.UTR5 | SCLC-I | F | chr16: 89438503- | S_Shelf |
| | | | | | 89438850 | |
| cg02628840 | chr21: 46860579 | COL18A1.GENEBODY | SCLC-I | R | chr21: 46860366- | Island |
| | | | | | 46860628 | |
| cg06712559 | chr1: 968395 | AGRN.GENEBODY | SCLC-I | F | chr1: 967966-970238 | Island |
| cg09235583 | chr6: 30711141 | IER3.UTR3 | SCLC-I | F | chr6: 30710307- | Island |
| | | | | | 30712440 | |
| cg20931195 | chr17: 708009 | NXN.GENEBODY | SCLC-I | F | chr17: 708319-708598 | N_Shore |
| cg25549619 | chr5: 147154246 | JAKMIP2.UTR5 | SCLC-I | F | | |
| cg27480819 | chr12: 23686149 | SOX5.UTR3 | SCLC-I | R | | |
| cg00201445 | chr3: 192634310 | C3orf59.GENEBODY | SCLC-I | R | chr3: 192634309- | Island |
| | | | | | 192635967 | |
| cg00650953 | chr7: 147500503 | CNTNAP2.GENEBODY | SCLC-I | R | | |
| cg00698688 | chr19: 49055432 | SULT2B1.EXON1 | SCLC-I | F | | |
| cg00965643 | chr1: 32674817 | DCDC2B.EXON1 | SCLC-I | F | chr1: 32670569- | S_Shelf |
| | | | | | 32671885 | |
| cg00974598 | chr19: 44223902 | IRGC.GENEBODY | SCLC-I | R | chr19: 44222858- | Island |
| | | | | | 44223943 | |
| cg01030534 | chr7: 143579698 | FAM115A.UTR5 | SCLC-I | F | chr7: 143582125- | N_Shelf |
| | | | | | 143582610 | |
| cg01243297 | chr6: 33160292 | COL11A2.TSS200 | SCLC-I | F | chr6: 33160945- | N_Shore |
| | | | | | 33161337 | |
| cg02633149 | chr17: 81042782 | METRNL.GENEBODY | SCLC-I | R | chr17: 81042626- | Island |
| | | | | | 81043244 | |
| cg03039843 | chr19: 49055390 | SULT2B1.TSS200 | SCLC-I | R | | |
| cg03485217 | chr4: 10686300 | CLNK.EXON1 | SCLC-I | R | | |
| cg03607415 | chr5: 89881642 | GPR98.GENEBODY | SCLC-I | R | | |
| cg03761471 | chr7: 143077178 | ZYX.TSS1500 | SCLC-I | F | chr7: 143077469- | N_Shore |
| | | | | | 143079169 | |
| cg03936135 | chr16: 86371248 | LOC732275.GENEBODY | SCLC-I | F | | |
| cg04601383 | chr19: 6713459 | C3.GENEBODY | SCLC-I | R | chr19: 6710625-6710828 | S_Shelf |
| cg05681757 | chr12: 32655034 | FGD4.TSS200 | SCLC-I | R | | |
| cg06822340 | chr7: 147355839 | CNTNAP2.GENEBODY | SCLC-I | R | | |
| cg06852032 | chr17: 25976191 | LGALS9.GENEBODY | SCLC-I | F | | |
| cg07001963 | chr10: 44879465 | CXCL12.GENEBODY | SCLC-I | R | chr10: 44879714- | N_Shore |
| | | | | | 44882391 | |
| cg07095230 | chr17: 59480730 | TBX2.GENEBODY | SCLC-I | F | chr17: 59473060- | Island |
| | | | | | 59483266 | |
| cg07152605 | chr1: 164793520 | PBX1.GENEBODY | SCLC-I | R | | |
| cg07270851 | chr11: 65779332 | CST6.TSS200 | SCLC-I | F | chr11: 65779311- | Island |
| | | | | | 65779767 | |
| cg07287698 | chr12: 97957454 | MIR135A2.TSS200 | SCLC-I | F | | |
| cg08787401 | chr2: 222383520 | EPHA4.GENEBODY | SCLC-I | F | | |
| cg09225457 | chr7: 8479893 | NXPH1.GENEBODY | SCLC-I | R | chr7: 8481974-8482762 | N_Shelf |
| cg09932135 | chr1: 11750569 | MAD2L2.UTR5 | SCLC-I | F | chr1: 11750701- | N_Shore |
| | | | | | 11752651 | |
| cg11904429 | chr19: 6592554 | CD70.TSS1500 | SCLC-I | F | chr19: 6590340-6591072 | S_Shore |
| cg12197421 | chr6: 30653736 | KIAA1949.GENEBODY | SCLC-I | F | chr6: 30654392- | N_Shore |
| | | | | | 30654934 | |
| cg12747860 | chr6: 3229027 | TUBB2B.TSS1500 | SCLC-I | R | chr6: 3227025-3229688 | Island |
| cg12805420 | chr17: 7165840 | CLDN7.TSS200 | SCLC-I | R | chr17: 7164285-7166245 | Island |
| cg12967164 | chr7: 1753637 | ELFN1.UTR5 | SCLC-I | F | chr7: 1753446-1753706 | Island |
| cg13094252 | chr8: 104153753 | C8orf56.TSS200 | SCLC-I | R | chr8: 104152726- | S_Shore |
| | | | | | 104153390 | |
| cg13673837 | chr19: 15306670 | NOTCH3.GENEBODY | SCLC-I | F | chr19: 15306243- | Island |
| | | | | | 15307111 | |
| cg14282850 | chr10: 123924917 | TACC2.GENEBODY | SCLC-I | F | chr10: 123922850- | S_Shore |
| | | | | | 123923542 | |
| cg14347177 | chr11: 65779330 | CST6.TSS200 | SCLC-I | F | chr11: 65779311- | Island |
| | | | | | 65779767 | |
| cg14574369 | chr12: 57483631 | NAB2.GENEBODY | SCLC-I | R | chr12: 57480797- | S_Shore |
| | | | | | 57483116 | |
| cg14663984 | chr1: 969042 | AGRN.GENEBODY | SCLC-I | F | chr1: 967966-970238 | Island |
| cg15633699 | chr1: 1356728 | LOC441869.TSS200 | SCLC-I | F | chr1: 1355159-1356000 | S_Shore |
| cg16663570 | chr8: 22014724 | LGI3.TSS1500 | SCLC-I | F | chr8: 22014152- | S_Shore |
| | | | | | 22014559 | |
| cg16713727 | chr1: 16062720 | SLC25A34.TSS200 | SCLC-I | R | | |
| cg17928920 | chr1: 968558 | AGRN.GENEBODY | SCLC-I | R | chr1: 967966-970238 | Island |
| cg18425731 | chr2: 158184641 | ERMN.TSS1500 | SCLC-I | F | | |
| cg19092045 | chr13: 54887369 | MIR1297.TSS1500 | SCLC-I | R | | |
| cg19244428 | chr6: 3229233 | TUBB2B.TSS1500 | SCLC-I | R | chr6: 3227025-3229688 | Island |
| cg19791630 | chr19: 6772811 | VAV1.UTR5 | SCLC-I | F | | |
| cg20308663 | chr13: 100523121 | CLYBL.GENEBODY | SCLC-I | F | | |
| cg20561300 | chr15: 93189780 | FAM174B.GENEBODY | SCLC-I | R | | |
| cg21131024 | chr8: 104153643 | C8orf56.TSS200 | SCLC-I | F | chr8: 104152726- | S_Shore |
| | | | | | 104153390 | |
| cg21422016 | chr7: 1753659 | ELFN1.UTR5 | SCLC-I | R | chr7: 1753446-1753706 | Island |
| cg22156128 | chr3: 10327453 | GHRL.GENEBODY | SCLC-I | R | | |
| cg22431093 | chr12: 32654992 | FGD4.TSS200 | SCLC-I | R | | |
| cg23049291 | chr1: 165204745 | LMX1A.GENEBODY | SCLC-I | F | chr1: 165204501- | Island |
| | | | | | 165205507 | |
| cg26080567 | chr21: 37536137 | DOPEY2.TSS1500 | SCLC-I | R | | |
| cg26190885 | chr19: 45409005 | APOE.TSS200 | SCLC-I | F | chr19: 45411720- | N_Shelf |
| | | | | | 45412600 | |
| cg26638341 | chr19: 1800126 | ATP8B3.GENEBODY | SCLC-I | F | chr19: 1799934-1800248 | Island |
| cg26737640 | chr19: 6587584 | CD70.GENEBODY | SCLC-I | F | chr19: 6590340-6591072 | N_Shelf |
| cg26946905 | chr16: 89441595 | ANKRD11.UTR5 | SCLC-I | F | chr16: 89438503- | S_Shelf |
| | | | | | 89438850 | |
| cg26993132 | chr16: 89239499 | CDH15.GENEBODY | SCLC-I | F | chr16: 89237944- | S_Shore |
| | | | | | 89238331 | |
| cg27082921 | chr19: 1800150 | ATP8B3.GENEBODY | SCLC-I | R | chr19: 1799934-1800248 | Island |
| cg02349785 | chr11: 65778967 | CST6.TSS1500 | SCLC-I | R | chr11: 65779311- | N_Shore |
| | | | | | 65779767 | |
| cg02576092 | chr1: 27668556 | SYTL1.EXON1 | SCLC-I | R | | |
| cg04327181 | chr11: 65779526 | CST6.EXON1 | SCLC-I | F | chr11: 65779311- | Island |
| | | | | | 65779767 | |
| cg04626868 | chr12: 23999034 | SOX5.GENEBODY | SCLC-I | R | | |
| cg05601974 | chr7: 150941389 | SMARCD3.GENEBODY | SCLC-I | F | chr7: 150942304- | N_Shore |
| | | | | | 150943033 | |
| cg09482611 | chr11: 65779263 | CST6.TSS200 | SCLC-I | R | chr11: 65779311- | N_Shore |
| | | | | | 65779767 | |
| cg14452346 | chr5: 147212032 | SPINK1.TSS1500 | SCLC-I | F | | |
| cg19296556 | chr19: 1802918 | ATP8B3.GENEBODY | SCLC-I | F | chr19: 1799934-1800248 | S_Shelf |
| cg24478779 | chr2: 6110966 | LOC150622.GENEBODY | SCLC-I | R | | |
| cg04016660 | chr16: 85075232 | KIAA0513.UTR5 | SCLC-I | R | | |
| cg00107982 | chr22: 37813098 | ELFN2.UTR5 | SCLC-I | F | chr22: 37815803- | N_Shelf |
| | | | | | 37816477 | |
| cg00135874 | chr2: 210493473 | MAP2.UTR5 | SCLC-I | R | | |
| cg00464796 | chr15: 53852614 | WDR72.GENEBODY | SCLC-I | F | | |
| cg01084435 | chr16: 58535556 | NDRG4.GENEBODY | SCLC-I | F | chr16: 58535040- | Island |
| | | | | | 58535596 | |
| cg03992611 | chr15: 92397873 | SLCO3A1.GENEBODY | SCLC-I | R | chr15: 92396013- | S_Shore |
| | | | | | 92397682 | |
| cg04080448 | chr12: 97924358 | RMST.GENEBODY | SCLC-I | F | | |
| cg05393025 | chr1: 20141781 | RNF186.TSS200 | SCLC-I | R | | |
| cg06189653 | chr5: 176873332 | PRR7.TSS1500 | SCLC-I | R | chr5: 176873349- | N_Shore |
| | | | | | 176874737 | |
| cg06705685 | chr2: 8116961 | LOC339788.TSS200 | SCLC-I | F | | |
| cg08151612 | chr19: 49055438 | SULT2B1.EXON1 | SCLC-I | R | | |
| cg08966208 | chr11: 1769409 | HCCA2.GENEBODY | SCLC-I | F | chr11: 1768979-1770712 | Island |
| cg11406274 | chr6: 29856564 | HLA-H.GENEBODY | SCLC-I | F | chr6: 29855295- | Island |
| | | | | | 29856565 | |
| cg11736020 | chr6: 30651511 | KIAA1949.GENEBODY | SCLC-I | R | chr6: 30654392- | N_Shelf |
| | | | | | 30654934 | |
| cg11920519 | chr20: 33135025 | MAP1LC3A.UTR5 | SCLC-I | R | | |
| cg12570246 | chr7: 36959356 | MIR1200.TSS1500 | SCLC-I | F | | |
| cg12732715 | chr1: 148929740 | LOC645166.TSS1500 | SCLC-I | F | | |
| cg12781794 | chr11: 118505740 | PHLDB1.GENEBODY | SCLC-I | F | chr11: 118505508- | Island |
| | | | | | 118505741 | |
| cg13729891 | chr17: 7108468 | DLG4.GENEBODY | SCLC-I | F | chr17: 7108304-7108654 | Island |
| cg13951572 | chr8: 22409000 | SORBS3.TSS1500 | SCLC-I | F | chr8: 22408672- | Island |
| | | | | | 22409635 | |
| cg14089943 | chr10: 103990076 | PITX3.UTR3 | SCLC-I | R | chr10: 103990213- | N_Shore |
| | | | | | 103991829 | |
| cg14279361 | chr19: 6721955 | C3.TSS1500 | SCLC-I | R | | |
| cg14397690 | chr15: 60688948 | ANXA2.UTR5 | SCLC-I | F | chr15: 60689749- | N_Shore |
| | | | | | 60690430 | |
| cg14492061 | chr3: 63637129 | SNTN.TSS1500 | SCLC-I | R | | |
| cg14899716 | chr1: 42846987 | RIMKLA.GENEBODY | SCLC-I | F | chr1: 42845978- | Island |
| | | | | | 42846988 | |
| cg15249911 | chr19: 41634046 | CYP2F1.UTR3 | SCLC-I | R | chr19: 41633776- | Island |
| | | | | | 41634047 | |
| cg15507500 | chr19: 17605696 | SLC27A1.GENEBODY | SCLC-I | F | chr19: 17607983- | N_Shelf |
| | | | | | 17608406 | |
| cg16094352 | chr14: 24642317 | REC8.GENEBODY | SCLC-I | R | chr14: 24641053- | S_Shore |
| | | | | | 24642220 | |
| cg16318112 | chr1: 969254 | AGRN.GENEBODY | SCLC-I | F | chr1: 967966-970238 | Island |
| cg16706099 | chr11: 10059019 | FLJ32810.GENEBODY | SCLC-I | F | | |
| | 7 | | | | | |
| cg17058616 | chr17: 73102145 | SLC16A5.UTR3 | SCLC-I | R | chr17: 73105229- | N_Shelf |
| | | | | | 73106667 | |
| cg17610929 | chr2: 220379044 | ACCN4.UTR5 | SCLC-I | R | chr2: 220377743- | S_Shore |
| | | | | | 220377946 | |
| cg17826168 | chr19: 6464103 | CRB3.TSS200 | SCLC-I | F | chr19: 6463991-6464780 | Island |
| cg17945153 | chr6: 41978288 | CCND3.UTR5 | SCLC-I | R | | |
| cg19260567 | chr7: 147303836 | CNTNAP2.GENEBODY | SCLC-I | F | | |
| cg20855296 | chr17: 16255560 | CENPV.GENEBODY | SCLC-I | R | chr17: 16255618- | N_Shore |
| | | | | | 16257148 | |
| cg21061582 | chr18: 44498047 | PIAS2.TSS1500 | SCLC-I | F | chr18: 44496910- | S_Shore |
| | | | | | 44497832 | |
| cg21238061 | chr9: 104036809 | LPPR1.GENEBODY | SCLC-I | R | | |
| cg22365276 | chr15: 60688622 | ANXA2.UTR5 | SCLC-I | R | chr15: 60689749- | N_Shore |
| | | | | | 60690430 | |
| cg22376856 | chr1: 204643556 | LRRN2.UTR5 | SCLC-I | R | | |
| cg22862634 | chr11: 62369728 | EML3.UTR3 | SCLC-I | R | chr11: 62368454- | Island |
| | | | | | 62370491 | |
| cg23214395 | chr1: 20141778 | RNF186.TSS200 | SCLC-I | R | | |
| cg23265500 | chr14: 65016591 | C14orf50.TSS200 | SCLC-I | R | chr14: 65016516- | Island |
| | | | | | 65016909 | |
| cg23912509 | chr12: 97957406 | MIR135A2.TSS200 | SCLC-I | F | | |
| cg24556026 | chr9: 36258971 | GNE.TSS1500 | SCLC-I | F | chr9: 36258170- | S_Shore |
| | | | | | 36258886 | |
| cg24723883 | chr19: 2608495 | GNG7.UTR5 | SCLC-I | F | chr19: 2607725-2607967 | S_Shore |
| cg25124058 | chr12: 97886218 | RMST.GENEBODY | SCLC-I | F | | |
| cg26633373 | chr19: 54377836 | MYADM.UTR3 | SCLC-I | R | chr19: 54377172- | S_Shore |
| | | | | | 54377441 | |
| cg26660800 | chr17: 59480926 | TBX2.GENEBODY | SCLC-I | F | chr17: 59473060- | Island |
| | | | | | 59483266 | |
| cg00187437 | chr6: 31089157 | CDSN.TSS1500 | SCLC-I | R | | |
| cg00294550 | chr10: 62144786 | ANK3.GENEBODY | SCLC-I | F | | |
| cg02447471 | chr7: 95537015 | DYNC111.GENEBODY | SCLC-I | F | | |
| cg11243173 | chr12: 23999045 | SOX5.GENEBODY | SCLC-I | R | | |
| cg14894216 | chr11: 65794352 | CATSPER1.TSS1500 | SCLC-I | R | | |
| cg17295739 | chr6: 32049513 | TNXB.GENEBODY | SCLC-I | R | chr6: 32046815- | S_Shelf |
| | | | | | 32047094 | |
| cg24283921 | chr11: 10477461 | AMPD3.TSS200 | SCLC-I | F | | |
| cg26070020 | chr1: 165201385 | LMX1A.GENEBODY | SCLC-I | F | chr1: 165204501- | N_Shelf |
| | | | | | 165205507 | |
| cg27248041 | chr6: 93992464 | EPHA7.GENEBODY | SCLC-I | F | | |
| cg03514404 | chr7: 143579665 | FAM115A.UTR5 | SCLC-I | F | chr7: 143582125- | N_Shelf |
| | | | | | 143582610 | |
| cg00151914 | chr17: 34058550 | RASL10B.TSS200 | SCLC-I | R | chr17: 34058549- | Island |
| | | | | | 34058778 | |
| cg00721771 | chr3: 59903821 | FHIT.GENEBODY | SCLC-I | F | | |
| cg00784153 | chr19: 10077007 | COL5A3.GENEBODY | SCLC-I | R | chr19: 10077031- | N_Shore |
| | | | | | 10077444 | |
| cg00957688 | chr1: 36183672 | C1orf216.UTR5 | SCLC-I | F | chr1: 36184248- | N_Shore |
| | | | | | 36184774 | |
| cg01110620 | chr12: 113573312 | RASAL1.UTR5 | SCLC-I | F | chr12: 113572977- | Island |
| | | | | | 113573758 | |
| cg01538731 | chr16: 87757018 | KLHDC4.GENEBODY | SCLC-I | F | chr16: 87754008- | S_Shelf |
| | | | | | 87754285 | |
| cg01794802 | chr6: 31096931 | PSORS1C1.UTR5 | SCLC-I | R | | |
| cg02004418 | chr2: 45236740 | SIX2.TSS200 | SCLC-I | R | chr2: 45235511- | Island |
| | | | | | 45237792 | |
| cg02227919 | chr2: 171569132 | LOC440925.GENEBODY | SCLC-I | R | chr2: 171569877- | N_Shore |
| | | | | | 171573904 | |
| cg02849309 | chr6: 144386280 | PLAGL1.TSS1500 | SCLC-I | F | chr6: 144384894- | S_Shore |
| | | | | | 144385888 | |
| cg02916272 | chr11: 1769443 | HCCA2.GENEBODY | SCLC-I | F | chr11: 1768979-1770712 | Island |
| cg03153115 | chr19: 2604559 | GNG7.UTR5 | SCLC-I | F | chr19: 2607725-2607967 | N_Shelf |
| cg03207915 | chr6: 119669112 | MAN1A1.GENEBODY | SCLC-I | F | chr6: 119669589- | N_Shore |
| | | | | | 119671424 | |
| cg03546687 | chr16: 2198080 | RAB26.TSS1500 | SCLC-I | R | chr16: 2198455-2199129 | N_Shore |
| cg04396998 | chr19: 49376722 | PPP1R15A.GENEBODY | SCLC-I | R | chr19: 49375484- | S_Shore |
| | | | | | 49375928 | |
| cg04475868 | chr16: 86380594 | LOC732275.TSS1500 | SCLC-I | F | | |
| cg04742671 | chr19: 54781049 | LILRB2.GENEBODY | SCLC-I | R | | |
| cg05301811 | chr1: 20813666 | CAMK2N1.TSS1500 | SCLC-I | F | chr1: 20810462- | S_Shore |
| | | | | | 20813511 | |
| cg05484788 | chr20: 58180357 | PHACTR3.GENEBODY | SCLC-I | F | chr20: 58179808- | Island |
| | | | | | 58180787 | |
| cg06303936 | chr3: 172711298 | SPATA16.GENEBODY | SCLC-I | F | | |
| cg07629125 | chr13: 58299015 | PCDH17.GENEBODY | SCLC-I | R | | |
| cg08247297 | chr21: 46917541 | COL18A1.GENEBODY | SCLC-I | F | chr21: 46918188- | N_Shore |
| | | | | | 46918410 | |
| cg08269485 | chr11: 1769382 | HCCA2.GENEBODY | SCLC-I | F | chr11: 1768979-1770712 | Island |
| cg08550882 | chr11: 12308354 | MICALCL.TSS200 | SCLC-I | R | | |
| cg09708598 | chr13: 54887314 | MIR1297.TSS1500 | SCLC-I | F | | |
| cg10303653 | chr6: 32049516 | TNXB.GENEBODY | SCLC-I | R | chr6: 32046815- | S_Shelf |
| | | | | | 32047094 | |
| cg10560147 | chr11: 65779335 | CST6.TSS200 | SCLC-I | F | chr11: 65779311- | Island |
| | | | | | 65779767 | |
| cg12432038 | chr1: 247275735 | C1orf229.TSS200 | SCLC-I | R | chr1: 247274585- | Island |
| | | | | | 247275757 | |
| cg13064679 | chr6: 32049953 | TNXB.GENEBODY | SCLC-I | R | chr6: 32046815- | S_Shelf |
| | | | | | 32047094 | |
| cg13095737 | chr5: 177034964 | B4GALT7.GENEBODY | SCLC-I | R | chr5: 177035351- | N_Shore |
| | | | | | 177035660 | |
| cg15005677 | chr11: 45918197 | MAPK8IP1.GENEBODY | SCLC-I | R | chr11: 45921387- | N_Shelf |
| | | | | | 45922167 | |
| cg15144888 | chr11: 128760367 | KCNJ5.TSS1500 | SCLC-I | F | chr11: 128760441- | N_Shore |
| | | | | | 128761233 | |
| cg15323252 | chr8: 22436420 | PDLIM2.TSS1500 | SCLC-I | R | chr8: 22436295- | Island |
| | | | | | 22437076 | |
| cg16317181 | chr10: 85954559 | PCDH21.EXON1 | SCLC-I | R | chr10: 85954199- | Island |
| | | | | | 85955303 | |
| cg17765324 | chr12: 16359122 | SLC15A5.GENEBODY | SCLC-I | F | | |
| cg18320766 | chr19: 55598782 | EPS8L1.GENEBODY | SCLC-I | R | chr19: 55597977- | Island |
| | | | | | 55598887 | |
| cg19455335 | chr8: 22457658 | C8orf58.GENEBODY | SCLC-I | R | chr8: 22457123- | Island |
| | | | | | 22457753 | |
| cg19961803 | chr11: 65554928 | OVOL1.EXON1 | SCLC-I | F | chr11: 65553749- | Island |
| | | | | | 65555573 | |
| cg20383948 | chr21: 46898137 | COL18A1.GENEBODY | SCLC-I | R | chr21: 46897841- | Island |
| | | | | | 46898158 | |
| cg21475065 | chr7: 147336337 | CNTNAP2.GENEBODY | SCLC-I | R | | |
| cg22153696 | chr4: 186389425 | CCDC110.GENEBODY | SCLC-I | F | chr4: 186392574- | N_Shelf |
| | | | | | 186393184 | |
| cg22287800 | chr7: 8634300 | NXPH1.GENEBODY | SCLC-I | F | | |
| cg24042496 | chr11: 117497929 | DSCAML1.GENEBODY | SCLC-I | R | | |
| cg24990327 | chr7: 147591897 | MIR548F3.TSS1500 | SCLC-I | R | | |
| cg25317941 | chr2: 233351153 | ECEL1.GENEBODY | SCLC-I | R | chr2: 233350278- | Island |
| | | | | | 233352974 | |
| cg26104986 | chr11: 75275303 | SERPINH1.UTR5 | SCLC-I | F | chr11: 75272950- | S_Shore |
| | | | | | 75273616 | |
| cg27579097 | chr16: 734844 | JMJD8.TSS1500 | SCLC-I | R | chr16: 729438-735815 | Island |
| cg04486528 | chr7: 147727995 | MIR548F3.GENEBODY | SCLC-I | F | | |
| cg07047570 | chr20: 20350813 | INSM1.EXON1 | SCLC-I | F | chr20: 20344400- | S_Shore |
| | | | | | 20350605 | |
| cg07912147 | chr12: 26138851 | RASSF8.UTR5 | SCLC-I | R | | |
| cg09246103 | chr1: 8456466 | RERE.UTR5 | SCLC-I | R | | |
| cg09247061 | chr16: 88571844 | ZFPM1.GENEBODY | SCLC-I | F | chr16: 88573285- | N_Shore |
| | | | | | 88573489 | |
| cg09365529 | chr1: 75140289 | C1orf173.TSS1500 | SCLC-I | F | | |
| cg14950855 | chr12: 49740781 | DNAJC22.TSS1500 | SCLC-I | F | chr12: 49738680- | Island |
| | | | | | 49740841 | |
| cg15262242 | chr17: 47301741 | PHOSPHO1.GENEBODY | SCLC-I | R | chr17: 47301545- | Island |
| | | | | | 47302477 | |
| cg15802396 | chr17: 7108305 | DLG4.GENEBODY | SCLC-I | R | chr17: 7108304-7108654 | Island |
| cg16182479 | chr17: 41832466 | SOST.UTR3 | SCLC-I | R | chr17: 41832717- | N_Shore |
| | | | | | 41833195 | |
| cg16630229 | chr6: 33160604 | COL11A2.TSS1500 | SCLC-I | F | chr6: 33160945- | N_Shore |
| | | | | | 33161337 | |
| cg18395327 | chr2: 220379109 | ACCN4.EXON1 | SCLC-I | R | chr2: 220377743- | S_Shore |
| | | | | | 220377946 | |
| cg19822502 | chr6: 97456581 | KLHL32.GENEBODY | SCLC-I | F | | |
| cg22189307 | chr11: 61061699 | VWCE.GENEBODY | SCLC-I | R | chr11: 61061751- | N_Shore |
| | | | | | 61063063 | |
| cg23619910 | chr11: 10477452 | AMPD3.TSS200 | SCLC-I | R | | |
| cg24155984 | chr5: 66127678 | MAST4.GENEBODY | SCLC-I | F | | |
| cg26578983 | chr16: 85075249 | KIAA0513.UTR5 | SCLC-I | R | | |
| cg00691368 | chr6: 30909820 | DPCR1.GENEBODY | SCLC-I | F | | |
| cg00988129 | chr8: 75232556 | JPH1.GENEBODY | SCLC-I | R | chr8: 75232649- | N_Shore |
| | | | | | 75234101 | |
| cg03716590 | chr7: 95435309 | DYNC111.GENEBODY | SCLC-I | R | | |
| cg04770813 | chr6: 33160297 | COL11A2.TSS200 | SCLC-I | F | chr6: 33160945- | N_Shore |
| | | | | | 33161337 | |
| cg05273302 | chr19: 2095560 | C19orf36.TSS1500 | SCLC-I | F | chr19: 2095395-2095727 | Island |
| cg06212760 | chr1: 46664937 | POMGNT1.GENEBODY | SCLC-I | R | chr1: 46668821- | N_Shelf |
| | | | | | 46669258 | |
| cg07308243 | chr16: 85074963 | KIAA0513.UTR5 | SCLC-I | R | | |
| cg07409292 | chr4: 90228685 | GPRIN3.UTR5 | SCLC-I | R | chr4: 90228714- | N_Shore |
| | | | | | 90229010 | |
| cg08531687 | chr5: 89968232 | GPR98.GENEBODY | SCLC-I | F | | |
| cg09335647 | chr7: 147097440 | MIR54814.GENEBODY | SCLC-I | F | | |
| cg10332715 | chr1: 245017736 | HNRNPU.UTR3 | SCLC-I | F | | |
| cg10383447 | chr16: 58535093 | NDRG4.GENEBODY | SCLC-I | F | chr16: 58535040- | Island |
| | | | | | 58535596 | |
| cg10632765 | chr19: 6534916 | TNFSF9.GENEBODY | SCLC-I | F | chr19: 6534759-6535079 | Island |
| cg12046414 | chr2: 220379199 | ACCN4.EXON1 | SCLC-I | R | chr2: 220377743- | S_Shore |
| | | | | | 220377946 | |
| cg12489353 | chr19: 48231499 | EHD2.GENEBODY | SCLC-I | F | chr19: 48229072- | S_Shore |
| | | | | | 48229507 | |
| cg13455326 | chr19: 10043341 | OLFM2.GENEBODY | SCLC-I | R | chr19: 10046824- | N_Shelf |
| | | | | | 10047067 | |
| cg14098532 | chr3: 111717874 | TAGLN3.TSS200 | SCLC-I | F | | |
| cg14478768 | chr19: 1083429 | HMHA1.GENEBODY | SCLC-I | F | chr19: 1082576-1083430 | Island |
| cg15939253 | chr11: 66049932 | CNIH2.GENEBODY | SCLC-I | F | chr11: 66049750- | Island |
| | | | | | 66050229 | |
| cg16678522 | chr20: 32149963 | CBFA2T2.GENEBODY | SCLC-I | F | | |
| cg17831934 | chr7: 147264850 | CNTNAP2.GENEBODY | SCLC-I | R | | |
| cg17894318 | chr3: 181441384 | SOX2OT.GENEBODY | SCLC-I | F | chr3: 181444409- | N_Shelf |
| | | | | | 181445000 | |
| cg18512948 | chr14: 24641706 | REC8.EXON1 | SCLC-I | R | chr14: 24641053- | Island |
| | | | | | 24642220 | |
| cg19506253 | chr2: 158301839 | CYTIP.TSS1500 | SCLC-I | F | | |
| cg19702383 | chr12: 97885575 | RMST.GENEBODY | SCLC-I | F | | |
| cg19799744 | chr6: 46138963 | ENPP5.TSS1500 | SCLC-I | R | | |
| cg19924334 | chr20: 62197682 | PRIC285.GENEBODY | SCLC-I | F | chr20: 62193966- | Island |
| | | | | | 62198985 | |
| cg20716515 | chr19: 44172263 | PLAUR.GENEBODY | SCLC-I | F | chr19: 44172916- | N_Shore |
| | | | | | 44173252 | |
| cg21389753 | chr17: 59480674 | TBX2.GENEBODY | SCLC-I | F | chr17: 59473060- | Island |
| | | | | | 59483266 | |
| cg22881677 | chr12: 63195695 | PPM1H.GENEBODY | SCLC-I | R | chr12: 63195657- | Island |
| | | | | | 63195909 | |
| cg24743778 | chr2: 220379058 | ACCN4.UTR5 | SCLC-I | R | chr2: 220377743- | S_Shore |
| | | | | | 220377946 | |
| cg25242557 | chr11: 31833264 | PAX6.TSS1500 | SCLC-I | R | chr11: 31831620- | Island |
| | | | | | 31839038 | |
| cg25540824 | chr6: 30139686 | TRIM15.GENEBODY | SCLC-I | F | chr6: 30139718- | N_Shore |
| | | | | | 30140263 | |
| cg25587092 | chr6: 32076906 | TNXB.UTR5 | SCLC-I | R | | |
| cg27125631 | chr17: 47654653 | NXPH3.GENEBODY | SCLC-I | F | chr17: 47652789- | S_Shore |
| | | | | | 47653078 | |
| cg00936309 | chr6: 33141824 | COL11A2.GENEBODY | SCLC-I | F | | |
| cg01116568 | chr11: 10476529 | AMPD3.TSS200 | SCLC-I | R | chr11: 10472000- | S_Shelf |
| | | | | | 10472857 | |
| cg01594262 | chr19: 38806145 | YIF1B.GENEBODY | SCLC-I | R | chr19: 38806485- | N_Shore |
| | | | | | 38806747 | |
| cg02355653 | chr6: 29710830 | LOC285830.GENEBODY | SCLC-I | R | | |
| cg02452416 | chr6: 33160450 | COL11A2.TSS1500 | SCLC-I | R | chr6: 33160945- | N_Shore |
| | | | | | 33161337 | |
| cg03054529 | chr7: 30029938 | SCRN1.TSS1500 | SCLC-I | F | chr7: 30028518- | S_Shore |
| | | | | | 30029822 | |
| cg04138361 | chr6: 33160304 | COL11A2.TSS200 | SCLC-I | F | chr6: 33160945- | N_Shore |
| | | | | | 33161337 | |
| cg04403415 | chr12: 124991087 | NCOR2.UTR5 | SCLC-I | F | | |
| cg07387813 | chr3: 40499408 | RPL14.GENEBODY | SCLC-I | R | chr3: 40498639- | S_Shore |
| | | | | | 40499260 | |
| cg08858649 | chr6: 30139903 | TRIM15.GENEBODY | SCLC-I | R | chr6: 30139718- | Island |
| | | | | | 30140263 | |
| cg09537107 | chr19: 48897008 | GRIN2D.TSS1500 | SCLC-I | F | chr19: 48896890- | Island |
| | | | | | 48897199 | |
| cg12049787 | chr19: 45409080 | APOE.EXON1 | SCLC-I | R | chr19: 45411720- | N_Shelf |
| | | | | | 45412600 | |
| cg12077433 | chr3: 159600838 | SCHIP1.GENEBODY | SCLC-I | F | | |
| cg12108912 | chr2: 120435949 | TMEM177.TSS1500 | SCLC-I | R | chr2: 120436530- | N_Shore |
| | | | | | 120437010 | |
| cg12643732 | chr5: 691580 | TPPP.UTR5 | SCLC-I | F | chr5: 692681-694377 | N_Shore |
| cg12776156 | chr11: 848044 | TSPAN4.UTR5 | SCLC-I | F | chr11: 849087-850982 | N_Shore |
| cg13212283 | chr12: 57632120 | NDUFA4L2.UTR5 | SCLC-I | F | chr12: 57631932- | Island |
| | | | | | 57634578 | |
| cg14507088 | chr9: 130690399 | PIP5KL1.GENEBODY | SCLC-I | R | chr9: 130689378- | S_Shore |
| | | | | | 130690215 | |
| cg15622556 | chr1: 12251820 | TNFRSF1B.GENEBODY | SCLC-I | F | chr1: 12251777- | Island |
| | | | | | 12251985 | |
| cg16458153 | chr6: 33160192 | COL11A2.UTR5 | SCLC-I | R | chr6: 33159991- | Island |
| | | | | | 33160263 | |
| cg16523839 | chr19: 10077134 | COL5A3.GENEBODY | SCLC-I | R | chr19: 10077031- | Island |
| | | | | | 10077444 | |
| cg16825133 | chr19: 675672 | FSTL3.TSS1500 | SCLC-I | R | chr19: 675671-675881 | Island |
| cg16860712 | chr19: 48244461 | EHD2.GENEBODY | SCLC-I | F | chr19: 48244150- | Island |
| | | | | | 48244693 | |
| cg18636319 | chr4: 26492206 | CCKAR.TSS200 | SCLC-I | R | | |
| cg18788725 | chr19: 45512122 | RELB.GENEBODY | SCLC-I | R | chr19: 45515163- | N_Shelf |
| | | | | | 45515560 | |
| cg19776793 | chr17: 79225974 | SLC38A10.GENEBODY | SCLC-I | F | | |
| cg20333836 | chr1: 156215418 | PAQR6.GENEBODY | SCLC-I | R | chr1: 156215368- | Island |
| | | | | | 156215899 | |
| cg20639396 | chr6: 128839906 | PTPRK.GENEBODY | SCLC-I | R | chr6: 128840158- | N_Shore |
| | | | | | 128842126 | |
| cg20672284 | chr12: 23998921 | SOX5.GENEBODY | SCLC-I | R | | |
| cg21009420 | chr7: 146484504 | CNTNAP2.GENEBODY | SCLC-I | R | | |
| cg23337031 | chr2: 236416064 | AGAP1.GENEBODY | SCLC-I | F | | |
| cg23627353 | chr8: 102669360 | GRHL2.GENEBODY | SCLC-I | F | | |
| cg26674800 | chr17: 7108653 | DLG4.GENEBODY | SCLC-I | F | chr17: 7108304-7108654 | Island |
| cg26853536 | chr12: 125399964 | UBC.TSS1500 | SCLC-I | F | chr12: 125398558- | S_Shore |
| | | | | | 125399899 | |
| cg00126034 | chr17: 44896162 | WNT3.TSS200 | SCLC-I | F | chr17: 44895800- | Island |
| | | | | | 44897025 | |
| cg00758881 | chr16: 58534681 | NDRG4.GENEBODY | SCLC-I | R | chr16: 58535040- | N_Shore |
| | | | | | 58535596 | |
| cg01029347 | chr14: 77606335 | ZDHHC22.UTR5 | SCLC-I | R | chr14: 77605885- | Island |
| | | | | | 77608304 | |
| cg02010738 | chr19: 2095466 | C19orf36.TSS1500 | SCLC-I | R | chr19: 2095395-2095727 | Island |
| cg03300078 | chr11: 1769522 | HCCA2.GENEBODY | SCLC-I | F | chr11: 1768979-1770712 | Island |
| cg04663692 | chr12: 97885669 | RMST.GENEBODY | SCLC-I | R | | |
| cg04694991 | chr10: 97219665 | SORBS1.UTR5 | SCLC-I | F | | |
| cg06998765 | chr14: 75389618 | RPS6KL1.TSS1500 | SCLC-I | F | chr14: 75389712- | N_Shore |
| | | | | | 75390133 | |
| cg07304692 | chr2: 100939045 | LONRF2.UTR5 | SCLC-I | R | chr2: 100937779- | Island |
| | | | | | 100939059 | |
| cg07775371 | chr16: 1922155 | C16orf73.UTR5 | SCLC-I | F | chr16: 1921897-1922426 | Island |
| cg09271709 | chr2: 50863603 | NRXN1.GENEBODY | SCLC-I | R | | |
| cg09327981 | chr7: 95455393 | DYNC111.GENEBODY | SCLC-I | F | | |
| cg09750510 | chr6: 25761690 | SLC17A4.UTR5 | SCLC-I | R | | |
| cg10098010 | chr6: 138483709 | KIAA1244.GENEBODY | SCLC-I | F | chr6: 138482534- | Island |
| | | | | | 138483710 | |
| cg10599438 | chr19: 844589 | PRTN3.GENEBODY | SCLC-I | F | chr19: 846011-846435 | N_Shore |
| cg11565299 | chr6: 30139478 | TRIM15.GENEBODY | SCLC-I | F | chr6: 30139718- | N_Shore |
| | | | | | 30140263 | |
| cg11775828 | chr2: 169100915 | STK39.GENEBODY | SCLC-I | F | chr2: 169102950- | N_Shelf |
| | | | | | 169104454 | |
| cg12281657 | chr14: 65016595 | C14orf50.TSS200 | SCLC-I | R | chr14: 65016516- | Island |
| | | | | | 65016909 | |
| cg12317456 | chr16: 55512868 | MMP2.TSS1500 | SCLC-I | R | chr16: 55513220- | N_Shore |
| | | | | | 55513526 | |
| cg12699599 | chr15: 40643142 | PHGR1.TSS200 | SCLC-I | R | | |
| cg12934382 | chr3: 51741135 | GRM2.EXON1 | SCLC-I | F | chr3: 51740740- | Island |
| | | | | | 51741413 | |
| cg13313836 | chr15: 60687284 | ANXA2.UTR5 | SCLC-I | R | chr15: 60689749- | N_Shelf |
| | | | | | 60690430 | |
| cg14307471 | chr18: 31432117 | NOL4.UTR3 | SCLC-I | F | | |
| cg15174248 | chr5: 149669674 | CAMK2A.TSS1500 | SCLC-I | F | | |
| cg17176573 | chr11: 120110887 | POU2F3.TSS200 | SCLC-I | R | chr11: 120110497- | S_Shore |
| | | | | | 120110719 | |
| cg19419650 | chr19: 7929643 | EVI5L.UTR3 | SCLC-I | F | chr19: 7926590-7928752 | S_Shore |
| cg19519964 | chr2: 223290012 | SGPP2.GENEBODY | SCLC-I | F | chr2: 223288922- | Island |
| | | | | | 223290013 | |
| cg19722656 | chr6: 119669372 | MAN1A1.GENEBODY | SCLC-I | R | chr6: 119669589- | N_Shore |
| | | | | | 119671424 | |
| cg20971970 | chr12: 57632495 | NDUFA4L2.UTR5 | SCLC-I | R | chr12: 57631932- | Island |
| | | | | | 57634578 | |
| cg21492961 | chr5: 90036436 | GPR98.GENEBODY | SCLC-I | R | | |
| cg21759280 | chr19: 6534936 | TNFSF9.GENEBODY | SCLC-I | F | chr19: 6534759-6535079 | Island |
| cg21958743 | chr12: 79832188 | SYT1.GENEBODY | SCLC-I | F | | |
| cg23084895 | chr1: 214162854 | PROX1.UTR5 | SCLC-I | R | chr1: 214160798- | S_Shore |
| | | | | | 214161034 | |
| cg25746092 | chr6: 31081617 | PSORS1C1.TSS1500 | SCLC-I | F | | |
| cg26576206 | chr19: 1064938 | ABCA7.GENEBODY | SCLC-I | F | chr19: 1064897-1065191 | Island |
| cg26839863 | chr10: 21814793 | C10orf140.TSS200 | SCLC-I | R | chr10: 21814786- | Island |
| | | | | | 21815716 | |
| cg26995690 | chr13: 36346376 | DCLK1.UTR3 | SCLC-I | F | | |
| cg00293644 | chr1: 32674341 | DCDC2B.TSS1500 | SCLC-I | R | chr1: 32670569- | S_Shelf |
| | | | | | 32671885 | |
| cg00407245 | chr19: 55598874 | EPS8L1.GENEBODY | SCLC-I | F | chr19: 55597977- | Island |
| | | | | | 55598887 | |
| cg00848007 | chr12: 68553396 | IFNG.EXON1 | SCLC-I | F | | |
| cg00956142 | chr1: 28765031 | PHACTR4.GENEBODY | SCLC-I | F | | |
| cg01666490 | chr8: 102984290 | NCALD.UTR5 | SCLC-I | R | | |
| cg01783706 | chr12: 49739991 | DNAJC22.TSS1500 | SCLC-I | R | chr12: 49738680- | Island |
| | | | | | 49740841 | |
| cg01902845 | chr1: 214170993 | PROX1.GENEBODY | SCLC-I | R | | |
| cg02275396 | chr19: 15554549 | WIZ.GENEBODY | SCLC-I | R | chr19: 15550849- | S_Shelf |
| | | | | | 15551059 | |
| cg03186999 | chr17: 7165420 | CLDN7.UTR5 | SCLC-I | F | chr17: 7164285-7166245 | Island |
| cg05257528 | chr19: 846179 | PRTN3.GENEBODY | SCLC-I | F | chr19: 846011-846435 | Island |
| cg06371583 | chr19: 2581343 | GNG7.UTR5 | SCLC-I | F | chr19: 2578956-2579746 | S_Shore |
| cg06764736 | chr12: 23998997 | SOX5.GENEBODY | SCLC-I | F | | |
| cg07299645 | chr7: 147256263 | MIR54814.TSS1500 | SCLC-I | F | | |
| cg07475922 | chr19: 10122694 | RDH8.TSS1500 | SCLC-I | R | chr19: 10120750- | S_Shore |
| | | | | | 10121231 | |
| cg08753391 | chr6: 32049622 | TNXB.GENEBODY | SCLC-I | R | chr6: 32046815- | S_Shelf |
| | | | | | 32047094 | |
| cg08897422 | chr7: 8666902 | NXPH1.GENEBODY | SCLC-I | F | | |
| cg09588360 | chr2: 54785795 | SPTBN1.UTR5 | SCLC-I | R | chr2: 54785026- | Island |
| | | | | | 54785969 | |
| cg10357885 | chr19: 10122000 | COL5A3.TSS1500 | SCLC-I | R | chr19: 10120750- | S_Shore |
| | | | | | 10121231 | |
| cg11289677 | chr6: 32049829 | TNXB.GENEBODY | SCLC-I | R | chr6: 32046815- | S_Shelf |
| | | | | | 32047094 | |
| cg11620475 | chr5: 135529232 | LOC389332.TSS1500 | SCLC-I | R | chr5: 135527056- | S_Shore |
| | | | | | 135528978 | |
| cg11742103 | chr11: 62369870 | EML3.UTR3 | SCLC-I | R | chr11: 62368454- | Island |
| | | | | | 62370491 | |
| cg13500113 | chr2: 216947149 | TMEM169.UTR5 | SCLC-I | R | chr2: 216946324- | S_Shore |
| | | | | | 216946734 | |
| cg13711424 | chr14: 65017091 | C14orf50.GENEBODY | SCLC-I | R | chr14: 65016516- | S_Shore |
| | | | | | 65016909 | |
| cg14025053 | chr8: 104153637 | C8orf56.TSS200 | SCLC-I | F | chr8: 104152726- | S_Shore |
| | | | | | 104153390 | |
| cg15159069 | chr3: 192634089 | C3orf59.GENEBODY | SCLC-I | F | chr3: 192634309- | N_Shore |
| | | | | | 192635967 | |
| cg16573136 | chr12: 49740639 | DNAJC22.TSS1500 | SCLC-I | F | chr12: 49738680- | Island |
| | | | | | 49740841 | |
| cg16663341 | chr18: 42327562 | SETBP1.GENEBODY | SCLC-I | R | chr18: 42324454- | S_Shelf |
| | | | | | 42324966 | |
| cg17617491 | chr19: 2607726 | GNG7.UTR5 | SCLC-I | R | chr19: 2607725-2607967 | Island |
| cg17714703 | chr19: 4912221 | UHRF1.GENEBODY | SCLC-I | R | chr19: 4909262-4910256 | S_Shore |
| cg18010131 | chr11: 10476620 | AMPD3.TSS200 | SCLC-I | F | chr11: 10472000- | S_Shelf |
| | | | | | 10472857 | |
| cg18168101 | chr8: 104153627 | C8orf56.TSS200 | SCLC-I | F | chr8: 104152726- | S_Shore |
| | | | | | 104153390 | |
| cg18544974 | chr3: 132842314 | TMEM108.UTR5 | SCLC-I | R | | |
| cg18716083 | chr5: 111506418 | EPB41L4A.GENEBODY | SCLC-I | F | | |
| cg19132462 | chr11: 10476608 | AMPD3.TSS200 | SCLC-I | R | chr11: 10472000- | S_Shelf |
| | | | | | 10472857 | |
| cg19776942 | chr1: 32714164 | FAM167B.GENEBODY | SCLC-I | F | chr1: 32713918- | Island |
| | | | | | 32714186 | |
| cg19865770 | chr1: 221510889 | LOC400804.TSS1500 | SCLC-I | F | | |
| cg21171335 | chr12: 122356390 | WDR66.TSS200 | SCLC-I | F | chr12: 122356315- | Island |
| | | | | | 122356853 | |
| cg21213853 | chr3: 51741473 | GRM2.UTR5 | SCLC-I | F | chr3: 51740740- | S_Shore |
| | | | | | 51741413 | |
| cg21869609 | chr19: 2291613 | LINGO3.GENEBODY | SCLC-I | R | chr19: 2289895-2291904 | Island |
| cg22494035 | chr15: 74537819 | CCDC33.GENEBODY | SCLC-I | R | chr15: 74537809- | Island |
| | | | | | 74538114 | |
| cg22699676 | chr2: 171569187 | LOC440925.GENEBODY | SCLC-I | R | chr2: 171569877- | N_Shore |
| | | | | | 171573904 | |
| cg23601905 | chr17: 17661060 | RAI | SCLC-I | F | | |
| | | RAI1.UTR5 | | | | |
| cg23635599 | chr19: 45281006 | CBLC.TSS200 | SCLC-I | F | chr19: 45281133- | N_Shore |
| | | | | | 45281355 | |
| cg23816247 | chr8: 104153827 | BAALC.GENEBODY | SCLC-I | F | chr8: 104152726- | S_Shore |
| | | | | | 104153390 | |
| cg24852713 | chr19: 1083423 | HMHA1.GENEBODY | SCLC-I | F | chr19: 1082576-1083430 | Island |
| cg27103348 | chr21: 46321529 | ITGB2.GENEBODY | SCLC-I | R | chr21: 46321397- | Island |
| | | | | | 46321644 | |
| cg27273866 | chr12: 23919997 | SOX5.GENEBODY | SCLC-I | R | | |
| cg01337207 | chr6: 32063835 | TNXB.GENEBODY | SCLC-I | F | chr6: 32063533- | Island |
| | | | | | 32065044 | |
| cg02027561 | chr6: 126110790 | NCOA7.TSS1500 | SCLC-I | R | chr6: 126111537- | N_Shore |
| | | | | | 126112480 | |
| cg03601354 | chr8: 22408966 | SORBS3.TSS1500 | SCLC-I | F | chr8: 22408672- | Island |
| | | | | | 22409635 | |
| cg04015907 | chr20: 825489 | FAM110A.GENEBODY | SCLC-I | F | chr20: 825267-826276 | Island |
| cg05830220 | chr16: 87757033 | KLHDC4.GENEBODY | SCLC-I | F | chr16: 87754008- | S_Shelf |
| | | | | | 87754285 | |
| cg05957504 | chr5: 124014639 | ZNF608.GENEBODY | SCLC-I | F | | |
| cg06198398 | chr18: 44526707 | KATNAL2.TSS200 | SCLC-I | R | chr18: 44526866- | N_Shore |
| | | | | | 44527137 | |
| cg07312445 | chr4: 40752151 | NSUN7.UTR5 | SCLC-I | F | chr4: 40751842- | Island |
| | | | | | 40752493 | |
| cg07524919 | chr6: 32063901 | TNXB.GENEBODY | SCLC-I | F | chr6: 32063533- | Island |
| | | | | | 32065044 | |
| cg10578482 | chr8: 145111298 | OPLAH.GENEBODY | SCLC-I | R | chr8: 145111297- | Island |
| | | | | | 145111564 | |
| cg10625931 | chr12: 57632417 | NDUFA4L2.UTR5 | SCLC-I | R | chr12: 57631932- | Island |
| | | | | | 57634578 | |
| cg11632652 | chr3: 174648902 | NAALADL2.GENEBODY | SCLC-I | F | | |
| cg13788097 | chr3: 62467612 | CADPS.GENEBODY | SCLC-I | F | | |
| cg14188106 | chr6: 32063895 | TNXB.GENEBODY | SCLC-I | F | chr6: 32063533- | Island |
| | | | | | 32065044 | |
| cg15075267 | chr19: 15306332 | NOTCH3.GENEBODY | SCLC-I | F | chr19: 15306243- | Island |
| | | | | | 15307111 | |
| cg15857329 | chr8: 144653570 | C8orf73.GENEBODY | SCLC-I | F | chr8: 144653774- | N_Shore |
| | | | | | 144653976 | |
| cg16314401 | chr2: 73144481 | EMX1.TSS200 | SCLC-I | R | chr2: 73143055- | Island |
| | | | | | 73148260 | |
| cg16924391 | chr6: 29719411 | IFITM4P.TSS1500 | SCLC-I | F | chr6: 29720381- | N_Shore |
| | | | | | 29721101 | |
| cg16953031 | chr3: 175064540 | NAALADL2.GENEBODY | SCLC-I | F | | |
| cg17693604 | chr19: 3046805 | TLE2.UTR5 | SCLC-I | F | chr19: 3047224-3047444 | N_Shore |
| cg18612786 | chr19: 45281236 | CBLC.EXON1 | SCLC-I | R | chr19: 45281133- | Island |
| | | | | | 45281355 | |
| cg20502039 | chr7: 95804328 | SLC25A13.GENEBODY | SCLC-I | F | | |
| cg23288962 | chr13: 35517516 | NBEA.GENEBODY | SCLC-I | R | | |
| cg23563443 | chr8: 22408989 | SORBS3.TSS1500 | SCLC-I | F | chr8: 22408672- | Island |
| | | | | | 22409635 | |
| cg24836242 | chr6: 30908579 | DPCR1.TSS200 | SCLC-I | F | | |
| cg24847554 | chr3: 57530606 | DNAH12.TSS1500 | SCLC-I | F | | |
| cg27501759 | chr3: 8809715 | OXTR.GENEBODY | SCLC-I | F | chr3: 8808961-8811280 | Island |
| cg00378218 | chr17: 1546841 | SCARF1.GENEBODY | SCLC-I | F | chr17: 1546742-1547324 | Island |
| cg00637695 | chr5: 121759771 | SNCAIP.GENEBODY | SCLC-I | R | | |
| cg01101058 | chr14: 70235157 | SFRS5.GENEBODY | SCLC-I | F | chr14: 70233570- | S_Shore |
| | | | | | 70234668 | |
| cg01645995 | chr7: 92836152 | HEPACAM2.GENEBODY | SCLC-I | R | | |
| cg01664065 | chr13: 102249040 | ITGBL1.GENEBODY | SCLC-I | R | | |
| cg02097532 | chr3: 55673955 | ERC2.UTR3 | SCLC-I | F | | |
| cg02105326 | chr2: 239149142 | HES6.TSS1500 | SCLC-I | R | chr2: 239147592- | Island |
| | | | | | 239149900 | |
| cg02124724 | chr21: 46875142 | COL18A1.TSS1500 | SCLC-I | F | chr21: 46876168- | N_Shore |
| | | | | | 46876752 | |
| cg02515354 | chr7: 147256123 | MIR54814.TSS1500 | SCLC-I | F | | |
| cg02638523 | chr2: 236548728 | AGAP1.GENEBODY | SCLC-I | R | | |
| cg02754084 | chr12: 72338080 | TPH2.GENEBODY | SCLC-I | F | | |
| cg04657588 | chr17: 72427543 | GPRC5C.TSS200 | SCLC-I | F | chr17: 72426783- | Island |
| | | | | | 72428977 | |
| cg05301383 | chr2: 171569144 | LOC440925.GENEBODY | SCLC-I | F | chr2: 171569877- | N_Shore |
| | | | | | 171573904 | |
| cg06201680 | chr7: 151078646 | WDR86.UTR3 | SCLC-I | R | chr7: 151078462- | Island |
| | | | | | 151079536 | |
| cg07449967 | chr8: 56987020 | SNORD54.TSS1500 | SCLC-I | R | chr8: 56986556- | Island |
| | | | | | 56987308 | |
| cg07471157 | chr12: 49373263 | WNT1.GENEBODY | SCLC-I | R | chr12: 49371690- | Island |
| | | | | | 49375550 | |
| cg08580631 | chr2: 170220388 | LRP2.TSS1500 | SCLC-I | R | chr2: 170220772- | N_Shore |
| | | | | | 170221114 | |
| cg09114581 | chr6: 32077103 | TNXB.UTR5 | SCLC-I | R | | |
| cg10289744 | chr3: 271185 | CHL1.UTR5 | SCLC-I | R | | |
| cg10661054 | chr2: 128422179 | LIMS2.GENEBODY | SCLC-I | R | chr2: 128421719- | Island |
| | | | | | 128422182 | |
| cg10810657 | chr5: 66203228 | MAST4.GENEBODY | SCLC-I | F | | |
| cg10970409 | chr3: 42543566 | VIPR1.TSS1500 | SCLC-I | F | chr3: 42542820- | Island |
| | | | | | 42544524 | |
| cg11316386 | chr1: 247275808 | C1orf229.TSS200 | SCLC-I | R | chr1: 247274585- | S_Shore |
| | | | | | 247275757 | |
| cg11642377 | chr7: 8480779 | NXPH1.GENEBODY | SCLC-I | R | chr7: 8481974-8482762 | N_Shore |

TABLE 2
Top 1000 methylation sites assoaited with the SCLC-A subtype

| Methylation | Position | | | | Relation to |
|---|---|---|---|---|---|
| Site | (GRCh 37) | Gene | Strand | CpG Island Name | CpG Island |
| cg20728514 | chr22:44568423 | PARVG.TSS1500 | R | | |
| cg13429423 | chr22:44568775 | PARVG.TSS200 | F | | |
| cg04506569 | chr2:234837977 | TRPM8.GENEBODY | F | | |
| cg17542751 | chr22:44568725 | PARVG.TSS200 | R | | |
| cg12688942 | chr1:19680353 | CAPZB.GENEBODY | R | | |
| cg25353896 | chr22:44568753 | PARVG.TSS200 | F | | |
| cg02911509 | chr22:44568770 | PARVG.TSS200 | F | | |
| cg16873414 | chr22:44568699 | PARVG.TSS200 | R | | |
| cg25901559 | chr7:157585255 | PTPRN2.GENEBODY | F | chr7:157586711-157587112 | N_Shore |
| cg07639982 | chr10:12555079 | CAMK1D.GENEBODY | F | | |
| cg08271031 | chr22:44568812 | PARVG.TSS200 | R | | |
| cg17622855 | chr6:157982358 | ZDHHC14.GENEBODY | R | | |
| cg03817675 | chr7:139299053 | HIPK2.GENEBODY | R | | |
| cg17252884 | chr3:15369681 | SH3BP5.GENEBODY | F | chr3:15373314-15374209 | N_Shelf |
| cg07213482 | chr4:89660237 | FAM13A.GENEBODY | R | | |
| cg12907969 | chr5:89815060 | LYSMD3.GENEBODY | R | | |
| cg25694807 | chr2:161329607 | RBMS1.GENEBODY | F | | |
| cg00090674 | chr22:50446678 | IL17REL.UTR5 | F | chr22:50448567-50449521 | N_Shore |
| cg23317857 | chr6:41696297 | TFEB.UTR5 | F | | |
| cg11201256 | chr1:184403226 | C1orf21.UTR5 | R | | |
| cg16860004 | chr8:103137229 | NCALD.TSS1500 | F | chr8:103135914-103136775 | S_Shore |
| cg15892280 | chr21:40180000 | ETS2.UTR5 | F | chr21:40177002-40178667 | S_Shore |
| cg03568507 | chr2:240153791 | HDAC4.GENEBODY | F | chr2:240153476-240153792 | Island |
| cg19437757 | chr7:157697057 | PTPRN2.GENEBODY | R | chr7:157695025-157695358 | S_Shore |
| cg27550325 | chr7:157915584 | PTPRN2.GENEBODY | R | chr7:157918704-157919409 | N_Shelf |
| cg27122614 | chr7:157913741 | PTPRN2.GENEBODY | R | | |
| cg27110054 | chr8:103137274 | NCALD.TSS1500 | R | chr8:103135914-103136775 | S_Shore |
| cg07572930 | chr16:81527107 | CMIP.GENEBODY | F | | |
| cg17479501 | chr17:80806191 | TBCD.GENEBODY | R | | |
| cg02161503 | chr7:157955692 | PTPRN2.GENEBODY | R | chr7:157956911-157957273 | N_Shore |
| cg13287553 | chr7:4784419 | FOXK1.GENEBODY | F | chr7:4784820-4785058 | N_Shore |
| cg25746499 | chr2:240153778 | HDAC4.GENEBODY | F | chr2:240153476-240153792 | Island |
| cg10672201 | chr12:103353320 | ASCL1.UTR3 | F | chr12:103351579-103352695 | S_Shore |
| cg06043710 | chr10:560371 | DIP2C.GENEBODY | F | | |
| cg18966819 | chr7:4784325 | FOXK1.GENEBODY | R | chr7:4784820-4785058 | N_Shore |
| cg18578954 | chr6:28887247 | TRIM27.GENEBODY | F | chr6:28890951-28892013 | N_Shelf |
| cg06228260 | chr7:157854729 | PTPRN2.GENEBODY | R | | |
| cg03899215 | chr7:157996423 | PTPRN2.GENEBODY | R | chr7:157996642-157997027 | N_Shore |
| cg08946713 | chr2:191844998 | STAT1.GENEBODY | F | | |
| cg18424850 | chr10:132945786 | TCERG1L.GENEBODY | R | | |
| cg21167502 | chr14:104195666 | ZFYVE21.GENEBODY | R | chr14:104193901-104194627 | S_Shore |
| cg27119612 | chr15:57491001 | TCF12.GENEBODY | R | | |
| cg04921578 | chr13:101143257 | PCCA.GENEBODY | F | | |
| cg08277369 | chr16:84538885 | KIAA1609.TSS1500 | F | chr16:84538884-84539115 | Island |
| cg08448341 | chr6:134324873 | SLC2A12.GENEBODY | F | | |
| cg15193475 | chr15:80998711 | FAM108C1.GENEBODY | F | | |
| cg17468616 | chr1:226013212 | EPHX1.EXON1 | F | | |
| cg18730511 | chr22:50435413 | IL17REL.UTR3 | F | chr22:50438257-50438776 | N_Shelf |
| cg26239495 | chr11:122791447 | C11orf63.GENEBODY | R | | |
| cg01441025 | chr8:103136942 | NCALD.EXON1 | F | chr8:103135914-103136775 | S_Shore |
| cg15577272 | chr7:157932397 | PTPRN2.GENEBODY | F | | |
| cg21373996 | chr10:560669 | DIP2C.GENEBODY | R | | |
| cg06650067 | chr15:66084924 | DENND4A.TSS1500 | R | chr15:66083839-66084925 | Island |
| cg08951403 | chr7:157960420 | PTPRN2.GENEBODY | F | chr7:157956911-157957273 | S_Shelf |
| cg09500207 | chr11:68249304 | SAPS3.UTR5 | R | | |
| cg16896647 | chr9:93563776 | SYK.TSS1500 | R | chr9:93563775-93564546 | Island |
| cg10227678 | chr16:54096340 | FTO.GENEBODY | F | | |
| cg18883682 | chr7:36655667 | AOAH.GENEBODY | R | | |
| cg03558805 | chr7:73184883 | CLDN3.TSS1500 | F | chr7:73183379-73185115 | Island |
| cg03856153 | chr3:61904580 | PTPRG.GENEBODY | R | | |
| cg04610718 | chr12:103353362 | ASCL1.UTR3 | F | chr12:103351579-103352695 | S_Shore |
| cg06946543 | chr13:76377366 | LMO7.UTR5 | F | | |
| cg09845489 | chr7:157667853 | PTPRN2.GENEBODY | R | chr7:157666069-157666399 | S_Shore |
| cg10919624 | chr7:158279891 | PTPRN2.GENEBODY | F | chr7:158281373-158281734 | N_Shore |
| cg00799539 | chr12:103353183 | ASCL1.UTR3 | R | chr12:103351579-103352695 | S_Shore |
| cg06663149 | chr4:41457558 | LIMCH1.GENEBODY | F | | |
| cg13656878 | chr9:119048807 | PAPPA.GENEBODY | F | | |
| cg14207833 | chr19:18945153 | UPF1.GENEBODY | F | chr19:18942432-18943127 | S_Shelf |
| cg16762386 | chr1:27240514 | NR0B2.EXON1 | F | | |
| cg26350286 | chr8:95652691 | ESRP1.TSS1500 | R | chr8:95652455-95652873 | Island |
| cg00624799 | chr15:90605618 | ZNF710.UTR5 | R | | |
| cg03519907 | chr5:154092426 | LARP1.TSS200 | F | | |
| cg09176199 | chr1:246383021 | SMYD3.GENEBODY | F | | |
| cg15192120 | chr7:157626176 | PTPRN2.GENEBODY | R | | |
| cg16048383 | chr12:6483708 | SCNN1A.GENEBODY | R | | |
| cg22550299 | chr6:169654842 | THBS2.TSS1500 | R | | |
| cg23936766 | chr1:230406371 | GALNT2.GENEBODY | R | | |
| cg02390801 | chr15:74528565 | CCDC33.TSS200 | F | | |
| cg05503433 | chr19:42409117 | ARHGEF1.GENEBODY | F | chr19:42408315-42408535 | S_Shore |
| cg09259081 | chr16:84538889 | KIAA1609.TSS1500 | F | chr16:84538884-84539115 | Island |
| cg13547053 | chr2:179184864 | OSBPL6.TSS200 | R | | |
| cg25037165 | chr11:12824283 | TEAD1.GENEBODY | R | | |
| cg01187496 | chr5:167718124 | WWC1.TSS1500 | F | chr5:167718523-167719688 | N_Shore |
| cg06338523 | chr7:158279818 | PTPRN2.GENEBODY | F | chr7:158281373-158281734 | N_Shore |
| cg14947787 | chr14:65405931 | GPX2.UTR3 | F | | |
| cg16311186 | chr7:140114137 | RAB19.GENEBODY | F | | |
| cg17176609 | chr2:74135217 | ACTG2.GENEBODY | R | | |
| cg19648306 | chr2:179184854 | OSBPL6.TSS200 | R | | |
| cg19747960 | chr8:11269117 | C8orf12.GENEBODY | R | | |
| cg21867891 | chr18:56529835 | ZNF532.TSS1500 | R | chr18:56530395-56531288 | N_Shore |
| cg07124138 | chr16:88683903 | ZC3H18.GENEBODY | R | | |
| cg27336068 | chr10:1336103 | ADARB2.GENEBODY | R | | |
| cg19954227 | chr7:158113303 | PTPRN2.GENEBODY | F | chr7:158109334-158109594 | S_Shelf |
| cg20802509 | chr1:229764652 | URB2.GENEBODY | F | chr1:229761128-229762299 | S_Shelf |
| cg21679730 | chr1:65047485 | CACHD1.GENEBODY | R | | |
| cg21732383 | chr6:169654692 | THBS2.TSS1500 | F | | |
| cg02428178 | chr15:49104237 | CEP152.TSS1500 | R | chr15:49102731-49102938 | S_Shore |
| cg09450352 | chr7:157495481 | PTPRN2.GENEBODY | R | chr7:157494510-157494739 | S_Shore |
| cg13579752 | chr5:153577937 | GALNT10.GENEBODY | F | | |
| cg14647989 | chr22:43824766 | MPPED1.GENEBODY | R | chr22:43820887-43821136 | S_Shelf |
| cg14667175 | chr2:111623300 | ACOXL.GENEBODY | F | | |
| cg19212465 | chr7:157654170 | PTPRN2.GENEBODY | F | | |
| cg25029446 | chr7:157495282 | PTPRN2.GENEBODY | F | chr7:157494510-157494739 | S_Shore |
| cg00064840 | chr7:4784485 | FOXK1.GENEBODY | F | chr7:4784820-4785058 | N_Shore |
| cg02473460 | chr8:93074521 | RUNX1T1.GENEBODY | R | | |
| cg10880973 | chr6:157982213 | ZDHHC14.GENEBODY | R | | |
| cg15046304 | chr17:75205489 | SEC14L1.GENEBODY | F | chr17:75209459-75209672 | N_Shelf |
| cg22598247 | chr7:157614399 | PTPRN2.GENEBODY | R | chr7:157612036-157612316 | S_Shelf |
| cg26247373 | chr12:57655696 | R3HDM2.GENEBODY | F | | |
| cg00911446 | chr1:7520079 | CAMTA1.GENEBODY | F | | |
| cg12761282 | chr20:2276571 | TGM3.TSS200 | R | | |
| cg16645907 | chr5:154092360 | LARP1.TSS200 | F | | |
| cg22057874 | chr2:179184866 | OSBPL6.TSS200 | R | | |
| cg24928687 | chr1:226012913 | EPHX1.TSS200 | R | | |
| cg00668890 | chr12:6449990 | TNFRSF1A.GENEBODY | R | | |
| cg02901644 | chr2:234369787 | DGKD.GENEBODY | R | chr2:234369786-234370099 | Island |
| cg07696006 | chr15:74528563 | CCDC33.TSS200 | F | | |
| cg11575295 | chr7:158250911 | PTPRN2.GENEBODY | F | chr7:158247888-158249312 | S_Shore |
| cg15228639 | chr15:42289939 | PLA2G4E.GENEBODY | R | | |
| cg24241530 | chr7:158061820 | PTPRN2.GENEBODY | R | chr7:158060894-158061100 | S_Shore |
| cg27555529 | chr19:13617518 | CACNA1A.TSS1500 | F | chr19:13616752-13617267 | S_Shore |
| cg08758185 | chr5:150849936 | SLC36A1.GENEBODY | F | | |
| cg10899175 | chr2:11680144 | GREB1.EXON1 | F | | |
| cg15298059 | chr7:157668141 | PTPRN2.GENEBODY | F | chr7:157669879-157670095 | N_Shore |
| cg18635670 | chr6:26539327 | HMGN4.UTR5 | R | chr6:26538496-26539015 | S_Shore |
| cg20149780 | chr1:15751063 | EFHD2.GENEBODY | F | | |
| cg03877767 | chr2:11680057 | GREB1.UTR5 | F | | |
| cg10126234 | chr14:104195533 | ZFYVE21.GENEBODY | R | chr14:104193901-104194627 | S_Shore |
| cg11035519 | chr11:130051458 | ST14.GENEBODY | F | | |
| cg11281961 | chr2:97498902 | CNNM3.UTR3 | R | | |
| cg12098949 | chr17:75367442 | SEPT9.UTR5 | F | chr17:75368688-75370506 | N_Shore |
| cg18738906 | chr12:6483739 | SCNN1A.GENEBODY | R | | |
| cg24034277 | chr14:102467421 | DYNC1H1.GENEBODY | F | | |
| cg25264630 | chr7:157849744 | PTPRN2.GENEBODY | F | | |
| cg26849830 | chr12:109178373 | SSH1.UTR3 | R | | |
| cg06221087 | chr1:164659666 | PBX1.GENEBODY | F | | |
| cg06694040 | chr11:11301699 | GALNTL4.GENEBODY | F | | |
| cg07875069 | chr7:129985025 | CPA5.EXON1 | R | | |
| cg19308132 | chr8:103137173 | NCALD.TSS1500 | F | chr8:103135914-103136775 | S_Shore |
| cg19897017 | chr7:2163380 | MAD1L1.GENEBODY | F | | |
| cg27655671 | chr6:38207741 | BTBD9.GENEBODY | F | | |
| cg05800761 | chr18:56529852 | ZNF532.TSS1500 | F | chr18:56530395-56531288 | N_Shore |
| cg20371765 | chr7:8302271 | ICA1.TSS200 | F | chr7:8301031-8302252 | S_Shore |
| cg02716952 | chr7:148504681 | EZH2.UTR3 | R | | |
| cg14592260 | chr11:108798655 | DDX10.GENEBODY | F | | |
| cg16937735 | chr7:157562675 | PTPRN2.GENEBODY | R | chr7:157560974-157561195 | S_Shore |
| cg17208060 | chr2:11680086 | GREB1.EXON1 | R | | |
| cg22968401 | chr17:27370009 | PIPOX.UTR5 | F | | |
| cg23884690 | chr13:113678926 | MCF2L.GENEBODY | R | chr13:113676781-113677120 | S_Shore |
| cg27431545 | chr2:173607996 | RAPGEF4.GENEBODY | F | | |
| cg00283887 | chr7:157935557 | PTPRN2.GENEBODY | F | chr7:157937614-157937841 | N_Shelf |
| cg00773370 | chr12:6483772 | SCNN1A.GENEBODY | R | | |
| cg02842704 | chr5:151305746 | GLRA1.TSS1500 | R | chr5:151304226-151304824 | S_Shore |
| cg02865101 | chr7:157779894 | PTPRN2.GENEBODY | R | | |
| cg05579861 | chr7:157631040 | PTPRN2.GENEBODY | F | chr7:157631319-157631615 | N_Shore |
| cg06650260 | chr1:27240478 | NR0B2.EXON1 | R | | |
| cg07685977 | chr10:133036507 | TCERG1L.GENEBODY | F | | |
| cg13213923 | chr17:60885491 | MARCH10.UTR5 | R | chr17:60885408-60885857 | Island |
| cg19809865 | chr14:44976233 | FSCB.EXON1 | R | | |
| cg20986887 | chr6:28887284 | TRIM27.GENEBODY | R | chr6:28890951-28892013 | N_Shelf |
| cg23245485 | chr12:6484219 | SCNN1A.UTR5 | R | | |
| cg24085344 | chr17:47485676 | PHB.GENEBODY | R | | |
| cg25148025 | chr1:164681267 | PBX1.GENEBODY | F | | |
| cg00787726 | chr10:86113284 | FAM190B.UTR5 | F | | |
| cg03900492 | chr7:157345659 | PTPRN2.GENEBODY | R | chr7:157346613-157347069 | N_Shore |
| cg04892919 | chr2:191272604 | MFSD6.TSS1500 | R | chr2:191272484-191273671 | Island |
| cg07942479 | chr4:185747981 | ACSL1.TSS1500 | R | chr4:185746557-185747827 | S_Shore |
| cg00448761 | chr19:6066102 | RFX2.UTR5 | R | | |
| cg01792117 | chr17:27088227 | C17orf63.GENEBODY | F | | |
| cg02567323 | chr2:48671699 | KLRAQ1.GENEBODY | R | chr2:48667559-48668355 | S_Shelf |
| cg08932665 | chr11:71937515 | INPPL1.GENEBODY | F | chr11:71934247-71936321 | S_Shore |
| cg21380084 | chr7:157937891 | PTPRN2.GENEBODY | R | chr7:157937614-157937841 | S_Shore |
| cg23632237 | chr20:30560483 | XKR7.GENEBODY | F | chr20:30555901-30556502 | S_Shelf |
| cg25401010 | chr18:56529878 | ZNF532.TSS200 | F | chr18:56530395-56531288 | N_Shore |
| cg26312133 | chr17:71428204 | SDK2.GENEBODY | F | chr17:71431621-71431878 | N_Shelf |
| cg01321816 | chr15:91358514 | BLM.UTR3 | R | chr15:91362445-91362658 | N_Shelf |
| cg03653377 | chr7:158107573 | PTPRN2.GENEBODY | F | chr7:158109334-158109594 | N_Shore |
| cg24216770 | chr20:35091947 | DLGAP4.GENEBODY | F | chr20:35089448-35089998 | S_Shore |
| cg25766748 | chr19:13614882 | CACNA1A.GENEBODY | F | chr19:13616752-13617267 | N_Shore |
| cg26639747 | chr7:157590318 | PTPRN2.GENEBODY | R | chr7:157593887-157594128 | N_Shelf |
| cg27388763 | chr17:44648385 | ARL17A.GENEBODY | R | | |
| cg00362322 | chr16:88704230 | IL17C.TSS1500 | F | chr16:88706211-88706492 | N_Shore |
| cg04874261 | chr3:71773745 | EIF4E3.GENEBODY | F | chr3:71773826-71774873 | N_Shore |
| cg09130676 | chr7:158220856 | PTPRN2.GENEBODY | R | chr7:158217851-158220709 | S_Shore |
| cg18011401 | chr19:13617366 | CACNA1A.TSS200 | F | chr19:13616752-13617267 | S_Shore |
| cg19307406 | chr1:167874258 | ADCY10.UTR5 | R | | |
| cg19404354 | chr1:202093479 | GPR37L1.GENEBODY | F | | |
| cg19637113 | chr15:74528522 | CCDC33.TSS200 | F | | |
| cg19726712 | chr8:98790327 | LAPTM4B.GENEBODY | F | chr8:98787835-98788874 | S_Shore |
| cg23969274 | chr6:116851736 | FAM26D.UTR5 | R | | |
| cg13024807 | chr6:53152718 | ELOVL5.GENEBODY | R | | |
| cg01100465 | chr7:157585095 | PTPRN2.GENEBODY | F | chr7:157586711-157587112 | N_Shore |
| cg04373993 | chr7:157570916 | PTPRN2.GENEBODY | F | chr7:157568163-157568404 | S_Shelf |
| cg05046377 | chr11:4927915 | OR51A7.TSS1500 | F | | |
| cg06160809 | chr2:217559688 | IGFBP5.UTR5 | F | chr2:217559039-217559484 | S_Shore |
| cg06676049 | chr2:23839842 | KLHL29.GENEBODY | R | | |
| cg06912665 | chr5:154092290 | LARP1.TSS200 | R | | |
| cg07927098 | chr7:157866704 | PTPRN2.GENEBODY | R | | |
| cg09644722 | chr17:60885519 | MARCH10.UTR5 | R | chr17:60885408-60885857 | Island |
| cg15726025 | chr1:32681995 | C1orf91.UTR3 | F | | |
| cg19188359 | chr20:2312099 | TGM3.GENEBODY | R | chr20:2308778-2309042 | S_Shelf |
| cg19890739 | chr16:85723539 | GINS2.TSS1500 | R | chr16:85722209-85722941 | S_Shore |
| cg23309969 | chr18:56529866 | ZNF532.TSS200 | F | chr18:56530395-56531288 | N_Shore |
| cg25507001 | chr5:137475288 | NME5.TSS200 | R | | |
| cg26546155 | chr16:57269635 | RSPRY1.GENEBODY | F | | |
| cg01520105 | chr6:111887243 | TRAF3IP2.GENEBODY | R | | |
| cg04911819 | chr10:24504859 | KIAA1217.GENEBODY | F | | |
| cg08471739 | chr3:129312902 | PLXND1.GENEBODY | F | | |
| cg15120341 | chr13:114238565 | TFDP1.TSS1500 | F | | |
| cg01277983 | chr22:43835600 | MPPED1.GENEBODY | F | | |
| cg01942646 | chr1:27240694 | NR0B2.TSS200 | R | | |
| cg02639667 | chr5:154092321 | LARP1.TSS200 | F | | |
| cg08353938 | chr2:217227472 | MARCH4.GENEBODY | R | | |
| cg26912251 | chr6:168970824 | SMOC2.GENEBODY | R | chr6:168972516-168974109 | N_Shore |
| cg03308120 | chr7:157933784 | PTPRN2.GENEBODY | F | chr7:157937614-157937841 | N_Shelf |
| cg07015608 | chr7:158114303 | PTPRN2.GENEBODY | R | chr7:158110569-158110881 | S_Shelf |
| cg08698159 | chr17:27294743 | SEZ6.GENEBODY | R | | |
| cg08725319 | chr7:157334751 | PTPRN2.GENEBODY | R | | |
| cg13531977 | chr9:112013420 | EPB41L4B.GENEBODY | R | | |
| cg19779605 | chr11:4703553 | OR51E2.GENEBODY | R | | |
| cg20448594 | chr7:73940352 | GTF2IRD1.GENEBODY | R | | |
| cg21857959 | chr3:182896995 | MCF2L2.UTR3 | F | chr3:182896920-182897381 | Island |
| cg26572499 | chr15:22929910 | CYFIP1.GENEBODY | R | | |
| cg04198210 | chr15:74528606 | CCDC33.TSS200 | F | | |
| cg05204104 | chr2:235403141 | ARL4C.UTR3 | R | chr2:235404502-235406541 | N_Shore |
| cg05822926 | chr7:8302235 | ICA1.TSS200 | F | chr7:8301031-8302252 | Island |
| cg18360873 | chr10:133051669 | TCERG1L.GENEBODY | F | | |
| cg00610577 | chr12:12008666 | ETV6.GENEBODY | R | | |
| cg07675656 | chr16:2105015 | TSC2.GENEBODY | R | chr16:2105014-2105390 | Island |
| cg11812748 | chr1:246733057 | CNST.UTR5 | F | chr1:246729207-246730276 | S_Shelf |
| cg14178173 | chr7:157495532 | PTPRN2.GENEBODY | F | chr7:157494510-157494739 | S_Shore |
| cg20724810 | chr3:182897196 | MCF2L2.GENEBODY | R | chr3:182896920-182897381 | Island |
| cg21831937 | chr15:57519802 | TCF12.GENEBODY | F | | |
| cg22049038 | chr7:146332766 | CNTNAP2.GENEBODY | R | | |
| cg23613317 | chr5:154092109 | LARP1.TSS1500 | R | | |
| cg24580782 | chr1:27240365 | NR0B2.EXON1 | R | | |
| cg25975382 | chr7:157532236 | PTPRN2.GENEBODY | F | | |
| cg01536803 | chr7:157968578 | PTPRN2.GENEBODY | F | | |
| cg06070324 | chr15:75322058 | PPCDC.GENEBODY | R | | |
| cg06413087 | chr7:157929609 | PTPRN2.GENEBODY | F | | |
| cg08203715 | chr11:126226154 | ST3GAL4.EXON1 | R | chr11:126225355-126226073 | S_Shore |
| cg15067687 | chr7:127658301 | SND1.GENEBODY | F | | |
| cg15428620 | chr10:102792835 | SFXN3.GENEBODY | F | chr10:102790820-102791049 | S_Shore |
| cg16681199 | chr6:70990268 | COL9A1.GENEBODY | R | chr6:70992040-70992912 | N_Shore |
| cg17976873 | chr14:23352295 | REM2.TSS200 | F | chr14:23355738-23356402 | N_Shelf |
| cg18717689 | chr9:116971578 | MIR455.TSS200 | R | | |
| cg25145687 | chr11:1606664 | HCCA2.GENEBODY | R | | |
| cg01154505 | chr2:112940409 | FBLN7.GENEBODY | F | chr2:112939287-112939563 | S_Shore |
| cg03071245 | chr13:37463034 | SMAD9.UTR5 | F | | |
| cg03527077 | chr7:158059704 | PTPRN2.GENEBODY | R | chr7:158060894-158061100 | N_Shore |
| cg07979652 | chr7:157934005 | PTPRN2.GENEBODY | R | chr7:157937614-157937841 | N_Shelf |
| cg09409457 | chr7:157954722 | PTPRN2.GENEBODY | F | chr7:157956911-157957273 | N_Shelf |
| cg13169065 | chr2:166650947 | GALNT3.TSS200 | F | chr2:166649909-166650966 | Island |
| cg13619044 | chr7:30202350 | C7orf41.UTR3 | R | | |
| cg15707428 | chr2:11679845 | GREB1.TSS1500 | R | | |
| cg16287740 | chr7:128595046 | TNPO3.UTR3 | R | | |
| cg19343518 | chr6:157508679 | ARID1B.GENEBODY | F | | |
| cg20632462 | chr11:20882850 | NELL1.GENEBODY | F | | |
| cg24129115 | chr14:23352488 | REM2.EXON1 | F | chr14:23355738-23356402 | N_Shelf |
| cg25439479 | chr8:92971562 | RUNX1T1.UTR3 | F | | |
| cg02873384 | chr11:4936857 | OR51G2.EXON1 | F | | |
| cg03892543 | chr16:84650330 | COTL1.GENEBODY | R | chr16:84650960-84651565 | N_Shore |
| cg04466743 | chr20:19915794 | RIN2.GENEBODY | F | | |
| cg06424550 | chr10:12808422 | CAMK1D.GENEBODY | R | | |
| cg08886790 | chr2:111857973 | ACOXL.GENEBODY | F | | |
| cg11959316 | chr1:57001742 | PPAP2B.GENEBODY | R | | |
| cg16396191 | chr13:42184765 | KIAA0564.GENEBODY | R | | |
| cg18352935 | chr11:4843021 | OR51F2.EXON1 | R | | |
| cg21185990 | chr7:157835049 | PTPRN2.GENEBODY | F | | |
| cg07056138 | chr8:103120260 | NCALD.UTR5 | F | | |
| cg07444408 | chr4:141218466 | SCOC.UTR5 | F | | |
| cg08871354 | chr16:85723489 | GINS2.TSS1500 | R | chr16:85722209-85722941 | S_Shore |
| cg09027472 | chr7:157981337 | PTPRN2.GENEBODY | F | chr7:157980786-157981462 | Island |
| cg11786338 | chr18:56809717 | SEC11C.GENEBODY | F | chr18:56806608-56807425 | S_Shelf |
| cg14537549 | chr10:12393185 | CAMK1D.GENEBODY | R | chr10:12390802-12392589 | S_Shore |
| cg16696476 | chr17:8191344 | RANGRF.TSS1500 | R | chr17:8191864-8192495 | N_Shore |
| cg20239333 | chr10:133111331 | TCERG1L.TSS1500 | F | chr10:133109193-133111052 | S_Shore |
| cg21088119 | chr8:11251471 | C8orf12.GENEBODY | F | | |
| cg23758354 | chr7:157434590 | PTPRN2.GENEBODY | R | | |
| cg27266257 | chr10:5707799 | ASB13.GENEBODY | F | chr10:5707980-5708798 | N_Shore |
| cg03345925 | chr8:144599347 | ZC3H3.GENEBODY | F | chr8:144601691-144601895 | N_Shelf |
| cg07998072 | chr16:19425853 | TMC5.UTR5 | F | chr16:19421816-19422498 | S_Shelf |
| cg08917718 | chr3:52002169 | PCBP4.TSS1500 | R | | |
| cg09127540 | chr7:157671453 | PTPRN2.GENEBODY | R | chr7:157669879-157670095 | S_Shore |
| cg10633463 | chr17:79197669 | AZI1.TSS1500 | R | chr17:79196345-79197419 | S_Shore |
| cg11803278 | chr7:157951799 | PTPRN2.GENEBODY | F | chr7:157951254-157951800 | Island |
| cg22797644 | chr6:34206491 | HMGA1.UTR5 | F | chr6:34202567-34206193 | S_Shore |
| cg22994649 | chr7:157641916 | PTPRN2.GENEBODY | R | | |
| cg00572756 | chr7:157863106 | PTPRN2.GENEBODY | R | | |
| cg02079584 | chr15:74528719 | CCDC33.EXON1 | F | | |
| cg02111655 | chr10:30338246 | KIAA1462.UTR5 | R | | |
| cg03332546 | chr19:16553887 | EPS15L1.GENEBODY | F | | |
| cg03660606 | chr7:157659983 | PTPRN2.GENEBODY | F | chr7:157659433-157660322 | Island |
| cg11857514 | chr12:122151762 | TMEM120B.GENEBODY | F | chr12:122150513-122151091 | S_Shore |
| cg14037240 | chr10:5707981 | ASB13.GENEBODY | R | chr10:5707980-5708798 | Island |
| cg17356036 | chr2:240213057 | HDAC4.GENEBODY | R | | |
| cg17985311 | chr1:42801965 | FOXJ3.TSS1500 | R | chr1:42800265-42801370 | S_Shore |
| cg19126954 | chr7:157616612 | PTPRN2.GENEBODY | F | chr7:157618856-157619302 | N_Shelf |
| cg25743482 | chr11:19775505 | NAV2.GENEBODY | F | | |
| cg02007867 | chr7:157786536 | PTPRN2.GENEBODY | R | | |
| cg06938143 | chr8:11251609 | C8orf12.GENEBODY | R | | |
| cg08850729 | chr1:60036715 | FGGY.GENEBODY | R | | |
| cg08989212 | chr21:36096051 | NCRNA00160.TSS200 | R | | |
| cg12193099 | chr6:169654018 | THBS2.UTR5 | R | | |
| cg12275410 | chr10:135123239 | ZNF511.GENEBODY | R | chr10:135123238-135123448 | Island |
| cg12356107 | chr20:22567037 | FOXA2.TSS1500 | R | chr20:22566821-22567055 | Island |
| cg15252509 | chr18:56337165 | MALT1.TSS1500 | R | chr18:56338211-56339277 | N_Shore |
| cg16289848 | chr4:79231477 | FRAS1.GENEBODY | F | | |
| cg00827176 | chr8:21885702 | NPM2.GENEBODY | R | chr8:21882245-21882883 | S_Shelf |
| cg01511231 | chr1:1109676 | TTLL10.UTR5 | F | chr1:1109314-1110145 | Island |
| cg08202399 | chr16:14280512 | MKL2.GENEBODY | F | | |
| cg14122506 | chr9:116060914 | RNF183.UTR5 | R | | |
| cg16934969 | chr2:173607848 | RAPGEF4.GENEBODY | R | | |
| cg19755171 | chr17:79197747 | AZI1.TSS1500 | R | chr17:79196345-79197419 | S_Shore |
| cg20649298 | chr20:22545407 | C20orf56.GENEBODY | R | chr20:22548967-22549720 | N_Shelf |
| cg22420514 | chr7:157997479 | PTPRN2.GENEBODY | F | chr7:157996642-157997027 | S_Shore |
| cg23997290 | chr20:22402746 | LOC284788.TSS1500 | F | | |
| cg27308682 | chr7:157637500 | PTPRN2.GENEBODY | F | | |
| cg01310118 | chr6:168855168 | SMOC2.GENEBODY | R | | |
| cg06155341 | chr11:66048759 | CNIH2.GENEBODY | R | chr11:66049750-66050229 | N_Shore |
| cg07143635 | chr16:11681487 | LITAF.TSS200 | F | chr16:11680008-11681275 | S_Shore |
| cg07551677 | chr8:28570561 | EXTL3.UTR5 | R | chr8:28573597-28573840 | N_Shelf |
| cg13071618 | chr8:144599125 | ZC3H3.GENEBODY | R | chr8:144601691-144601895 | N_Shelf |
| cg14706245 | chr7:157536727 | PTPRN2.GENEBODY | F | | |
| cg15175716 | chr11:64120685 | CCDC88B.GENEBODY | R | | |
| cg15504747 | chr3:129312979 | PLXND1.GENEBODY | R | | |
| cg20573218 | chr3:181409863 | SOX2OT.GENEBODY | F | chr3:181413014-181414022 | N_Shelf |
| cg20825710 | chr11:1620352 | HCCA2.GENEBODY | R | | |
| cg23869743 | chr10:99611477 | GOLGA7B.GENEBODY | F | chr10:99609425-99610116 | S_Shore |
| cg24119607 | chr5:139047739 | CXXC5.UTR5 | F | chr5:139047905-139048235 | N_Shore |
| cg25809301 | chr3:71179811 | FOXP1.GENEBODY | F | | |
| cg00781208 | chr12:64433590 | SRGAP1.GENEBODY | R | | |
| cg01282269 | chr21:41515306 | DSCAM.GENEBODY | F | chr21:41516237-41516472 | N_Shore |
| cg02098619 | chr1:8689772 | RERE.GENEBODY | R | | |
| cg02704103 | chr3:72846991 | SHQ1.GENEBODY | R | | |
| cg02704570 | chr7:157647109 | PTPRN2.GENEBODY | F | chr7:157647800-157648007 | N_Shore |
| cg04188351 | chr11:33744823 | CD59.UTR5 | R | | |
| cg04545268 | chr7:157653809 | PTPRN2.GENEBODY | F | | |
| cg04583813 | chr10:560323 | DIP2C.GENEBODY | F | | |
| cg06287775 | chr7:4784174 | FOXK1.GENEBODY | R | chr7:4784820-4785058 | N_Shore |
| cg06324554 | chr7:157495841 | PTPRN2.GENEBODY | R | chr7:157494510-157494739 | S_Shore |
| cg09215582 | chr4:71575507 | RUFY3.GENEBODY | R | chr4:71570147-71571626 | S_Shelf |
| cg16177693 | chr1:27240669 | NR0B2.TSS200 | R | | |
| cg18587271 | chr16:46917496 | GPT2.TSS1500 | F | chr16:46917849-46918957 | N_Shore |
| cg19149693 | chr3:114599007 | ZBTB20.UTR5 | F | | |
| cg23694187 | chr7:157335188 | PTPRN2.GENEBODY | F | | |
| cg23861617 | chr4:83422170 | TMEM150C.GENEBODY | F | | |
| cg00909926 | chr1:216774208 | ESRRG.GENEBODY | R | chr1:216774339-216774734 | N_Shore |
| cg00932063 | chr10:126234138 | LHPP.GENEBODY | R | | |
| cg02323550 | chr7:157660321 | PTPRN2.GENEBODY | F | chr7:157659433-157660322 | Island |
| cg03170878 | chr7:157630174 | PTPRN2.GENEBODY | F | chr7:157631319-157631615 | N_Shore |
| cg05104995 | chr10:49460249 | FRMPD2.GENEBODY | F | | |
| cg05336188 | chr19:18959506 | UPF1.GENEBODY | F | | |
| cg05987961 | chr7:158059396 | PTPRN2.GENEBODY | F | chr7:158060894-158061100 | N_Shore |
| cg11376706 | chr7:157514626 | PTPRN2.GENEBODY | F | | |
| cg15752885 | chr7:158255389 | PTPRN2.GENEBODY | F | | |
| cg19487718 | chr4:185747708 | ACSL1.TSS1500 | R | chr4:185746557-185747827 | Island |
| cg27282206 | chr7:158179313 | PTPRN2.GENEBODY | R | | |
| cg00108938 | chr7:157633252 | PTPRN2.GENEBODY | F | chr7:157631319-157631615 | S_Shore |
| cg11111139 | chr7:157620685 | PTPRN2.GENEBODY | R | chr7:157618856-157619302 | S_Shore |
| cg03347095 | chr7:158157967 | PTPRN2.GENEBODY | R | | |
| cg04772948 | chr20:22401407 | LOC284788.TSS200 | R | | |
| cg07011711 | chr11:61467286 | DAGLA.UTR5 | R | | |
| cg07037223 | chr7:157618857 | PTPRN2.GENEBODY | R | chr7:157618856-157619302 | Island |
| cg10798171 | chr7:8302301 | ICA1.TSS200 | F | chr7:8301031-8302252 | S_Shore |
| cg10817855 | chr7:99019985 | PTCD1.GENEBODY | F | chr7:99016967-99017289 | S_Shelf |
| cg10932907 | chr11:129723947 | TMEM45B.GENEBODY | F | | |
| cg12030523 | chr5:126984765 | CTXN3.EXON1 | R | | |
| cg13808071 | chr2:11679872 | GREB1.TSS1500 | R | | |
| cg19285478 | chr11:63849412 | MACROD1.GENEBODY | F | chr11:63852803-63853082 | N_Shelf |
| cg19564367 | chr4:7941852 | AFAP1.TSS200 | F | chr4:7940563-7941853 | Island |
| cg24324985 | chr20:22566884 | FOXA2.TSS1500 | R | chr20:22566821-22567055 | Island |
| cg26160008 | chr12:6451035 | TNFRSF1A.UTR5 | R | | |
| cg05358575 | chr2:26764961 | OTOF.GENEBODY | F | | |
| cg11855643 | chr1:184473341 | C1orf21.GENEBODY | R | | |
| cg13810707 | chr7:122018993 | CADPS2.GENEBODY | R | | |
| cg14974147 | chr3:121310923 | FBXO40.TSS1500 | F | | |
| cg00035220 | chr7:158197980 | PTPRN2.GENEBODY | F | chr7:158198545-158198907 | N_Shore |
| cg04112626 | chr4:79861272 | PAQR3.TSS1500 | F | chr4:79860213-79860745 | S_Shore |
| cg04873861 | chr3:12525454 | TSEN2.TSS1500 | F | chr3:12525749-12526346 | N_Shore |
| cg07011231 | chr2:73329813 | RAB11FIP5.GENEBODY | F | | |
| cg15000071 | chr3:134889493 | EPHB1.GENEBODY | F | | |
| cg15090562 | chr1:6534449 | PLEKHG5.GENEBODY | F | chr1:6530536-6531814 | S_Shelf |
| cg18187658 | chr1:230294691 | GALNT2.GENEBODY | R | | |
| cg23313445 | chr11:4792272 | OR51F1.TSS1500 | F | | |
| cg23636833 | chr7:157514822 | PTPRN2.GENEBODY | F | | |
| cg26713273 | chr12:11906243 | ETV6.GENEBODY | R | | |
| cg01570265 | chr14:70180751 | KIAA0247.UTR3 | R | | |
| cg03599855 | chr8:119073725 | EXT1.GENEBODY | R | | |
| cg04303033 | chr7:157586345 | PTPRN2.GENEBODY | F | chr7:157586711-157587112 | N_Shore |
| cg05241143 | chr7:157916636 | PTPRN2.GENEBODY | F | chr7:157918704-157919409 | N_Shelf |
| cg05430989 | chr3:133749309 | SLCO2A1.TSS1500 | F | chr3:133747920-133748266 | S_Shore |
| cg05998486 | chr7:150645092 | KCNH2.GENEBODY | R | chr7:150644455-150644767 | S_Shore |
| cg14171944 | chr9:126323477 | DENND1A.GENEBODY | F | | |
| cg21443274 | chr8:106782921 | ZFPM2.GENEBODY | R | | |
| cg11717251 | chr7:157589770 | PTPRN2.GENEBODY | F | chr7:157586711-157587112 | S_Shelf |
| cg25481630 | chr7:157499673 | PTPRN2.GENEBODY | R | chr7:157503469-157504144 | N_Shelf |
| cg03697708 | chr19:13617549 | CACNA1A.TSS1500 | F | chr19:13616752-13617267 | S_Shore |
| cg04983349 | chr6:80381447 | SH3BGRL2.GENEBODY | R | | |
| cg08570034 | chr5:175227702 | CPLX2.UTR5 | F | chr5:175223609-175224679 | S_Shelf |
| cg09805271 | chr11:5010562 | MMP26.GENEBODY | R | | |
| cg10531355 | chr5:79489249 | SERINC5.GENEBODY | R | | |
| cg13649658 | chr9:140680393 | EHMT1.GENEBODY | F | chr9:140683120-140683774 | N_Shelf |
| cg14779376 | chr7:157341829 | PTPRN2.GENEBODY | R | | |
| cg18769909 | chr7:157616107 | PTPRN2.GENEBODY | F | chr7:157618856-157619302 | N_Shelf |
| cg20808813 | chr7:157663646 | PTPRN2.GENEBODY | F | chr7:157666069-157666399 | N_Shelf |
| cg26562638 | chr1:226130366 | LEFTY2.TSS1500 | F | chr1:226127112-226127695 | S_Shelf |
| cg01863682 | chr2:182545771 | NEUROD1.TSS1500 | R | chr2:182547873-182549177 | N_Shelf |
| cg02573468 | chr15:41224537 | DLL4.GENEBODY | F | chr15:41217789-41223180 | S_Shore |
| cg02860921 | chr7:157506297 | PTPRN2.GENEBODY | F | chr7:157503469-157504144 | S_Shelf |
| cg04087039 | chr7:157697459 | PTPRN2.GENEBODY | R | chr7:157694387-157694661 | S_Shelf |
| cg06455686 | chr6:168971081 | SMOC2.GENEBODY | F | chr6:168972516-168974109 | N_Shore |
| cg10122877 | chr4:37585361 | C4orf19.TSS1500 | F | | |
| cg10288307 | chr7:157563602 | PTPRN2.GENEBODY | R | chr7:157560974-157561195 | S_Shelf |
| cg11261691 | chr20:30914444 | KIF3B.GENEBODY | R | | |
| cg13915179 | chr7:158038007 | PTPRN2.GENEBODY | R | chr7:158037708-158038048 | Island |
| cg19280540 | chr19:16553711 | EPS15L1.GENEBODY | R | | |
| cg22053861 | chr11:65318683 | LTBP3.GENEBODY | R | chr11:65321225-65321823 | N_Shelf |
| cg22705918 | chr2:25518238 | DNMT3A.GENEBODY | F | | |
| cg24060173 | chr1:205488243 | CDK18.UTR5 | R | | |
| cg25001924 | chr7:157919982 | PTPRN2.GENEBODY | R | chr7:157918704-157919409 | S_Shore |
| cg26816432 | chr7:158165951 | PTPRN2.GENEBODY | F | | |
| cg27099497 | chr7:158269297 | PTPRN2.GENEBODY | F | | |
| cg27324698 | chr7:157916687 | PTPRN2.GENEBODY | F | chr7:157918704-157919409 | N_Shelf |
| cg01181415 | chr12:16757954 | LMO3.UTR5 | R | | |
| cg05552035 | chr8:37699239 | GPR124.GENEBODY | F | chr8:37698846-37699931 | Island |
| cg06818217 | chr12:97887662 | RMST.GENEBODY | R | | |
| cg08141495 | chr18:31739521 | NOL4.GENEBODY | F | chr18:31739047-31739522 | Island |
| cg09643186 | chr14:65409452 | GPX2.EXON1 | F | | |
| cg13432032 | chr15:76692444 | SCAPER.GENEBODY | F | | |
| cg17611686 | chr20:22547532 | C20orf56.GENEBODY | F | chr20:22548967-22549720 | N_Shore |
| cg20821442 | chr7:105279882 | ATXN7L1.GENEBODY | F | | |
| cg24040043 | chr1:207224982 | YOD1.TSS1500 | F | chr1:207226281-207226842 | N_Shore |
| cg24524099 | chr7:157643007 | PTPRN2.GENEBODY | R | | |
| cg02546690 | chr11:17834480 | SERGEF.GENEBODY | R | | |
| cg02932780 | chr6:31762353 | VARS.GENEBODY | R | chr6:31763240-31763905 | N_Shore |
| cg04983296 | chr4:79861284 | PAQR3.TSS1500 | F | chr4:79860213-79860745 | S_Shore |
| cg09367807 | chr11:1577706 | HCCA2.GENEBODY | R | chr11:1577705-1579091 | Island |
| cg11199014 | chr19:39798563 | LRFN1.GENEBODY | R | chr19:39797786-39799250 | Island |
| cg14468481 | chr7:157561104 | PTPRN2.GENEBODY | R | chr7:157560974-157561195 | Island |
| cg21759685 | chr3:46400618 | CCR2.UTR3 | F | | |
| cg23629187 | chr8:125576109 | MTSS1.GENEBODY | R | | |
| cg00341732 | chr22:31479561 | SMTN.GENEBODY | F | chr22:31480774-31481373 | N_Shore |
| cg00362285 | chr1:236444212 | ERO1LB.GENEBODY | F | chr1:236444603-236445700 | N_Shore |
| cg01387450 | chr2:23839710 | KLHL29.GENEBODY | R | | |
| cg01557792 | chr14:70162755 | KIAA0247.GENEBODY | F | | |
| cg07926025 | chr9:14722864 | CER1.TSS200 | R | | |
| cg11939450 | chr20:18024215 | OVOL2.GENEBODY | R | | |
| cg14119936 | chr11:4791012 | OR51F1.EXON1 | F | | |
| cg15727524 | chr7:157531216 | PTPRN2.GENEBODY | F | | |
| cg16615040 | chr21:41516861 | DSCAM.GENEBODY | R | chr21:41516237-41516472 | S_Shore |
| cg17491449 | chr4:3344154 | RGS12.GENEBODY | F | chr4:3341783-3342021 | S_Shelf |
| cg17599372 | chr7:157894874 | PTPRN2.GENEBODY | F | chr7:157890656-157891557 | S_Shelf |
| cg22016731 | chr1:40311142 | TRIT1.GENEBODY | F | | |
| cg22792988 | chr5:76376904 | SNORA47.TSS1500 | F | chr5:76372882-76373720 | S_Shelf |
| cg23919844 | chr13:111819481 | ARHGEF7.GENEBODY | R | | |
| cg25680145 | chr6:125623414 | HDDC2.TSS200 | F | chr6:125622704-125623117 | S_Shore |
| cg26480662 | chr7:157778593 | PTPRN2.GENEBODY | R | | |
| cg26674966 | chr15:22929993 | CYFIP1.GENEBODY | R | | |
| cg01782132 | chr16:28936054 | RABEP2.GENEBODY | R | chr16:28936053-28937036 | Island |
| cg03788215 | chr18:76757841 | SALL3.UTR3 | F | chr18:76751939-76755549 | S_Shelf |
| cg06899237 | chr6:153353519 | RGS17.GENEBODY | R | | |
| cg08278648 | chr17:27503704 | MYO18A.UTR5 | R | chr17:27503598-27504014 | Island |
| cg08450080 | chr7:157919807 | PTPRN2.GENEBODY | R | chr7:157918704-157919409 | S_Shore |
| cg12699647 | chr17:80401857 | C17orf62.UTR3 | R | chr17:80401644-80401858 | Island |
| cg13917127 | chr9:116932436 | COL27A1.GENEBODY | F | chr9:116930004-116930368 | S_Shelf |
| cg15578140 | chr7:147718109 | MIR548F3.GENEBODY | R | | |
| cg17323383 | chr3:182896670 | MCF2L2.UTR3 | F | chr3:182896920-182897381 | N_Shore |
| cg18284875 | chr7:157777033 | PTPRN2.GENEBODY | F | | |
| cg20018344 | chr20:3204138 | ITPA.UTR3 | R | | |
| cg21733013 | chr7:22359568 | RAPGEF5.UTR5 | R | | |
| cg23606244 | chr11:4704115 | OR51E2.UTR5 | F | | |
| cg23808337 | chr7:158267402 | PTPRN2.GENEBODY | R | | |
| cg24424217 | chr10:135123421 | ZNF511.GENEBODY | F | chr10:135123238-135123448 | Island |
| cg25455408 | chr19:42274924 | CEACAM6.UTR3 | R | | |
| cg17015844 | chr12:103352694 | ASCL1.EXON1 | F | chr12:103351579-103352695 | Island |
| cg00574412 | chr17:27892866 | ABHD15.GENEBODY | F | chr17:27893086-27896078 | N_Shore |
| cg05012972 | chr7:158250129 | PTPRN2.GENEBODY | R | chr7:158250128-158250497 | Island |
| cg10801633 | chr1:10579190 | PEX14.GENEBODY | R | | |
| cg11623476 | chr7:2566027 | LFNG.GENEBODY | F | chr7:2565872-2566114 | Island |
| cg12661624 | chr7:157957646 | PTPRN2.GENEBODY | F | chr7:157956911-157957273 | S_Shore |
| cg13102244 | chr7:158198569 | PTPRN2.GENEBODY | R | chr7:158198545-158198907 | Island |
| cg14527645 | chr9:116977924 | COL27A1.GENEBODY | F | | |
| cg14670303 | chr6:169654035 | THBS2.UTR5 | R | | |
| cg18280382 | chr11:126225996 | ST3GAL4.TSS200 | F | chr11:126225355-126226073 | Island |
| cg18438823 | chr11:4676365 | OR51E1.UTR3 | R | | |
| cg19268477 | chr7:157951471 | PTPRN2.GENEBODY | F | chr7:157951254-157951800 | Island |
| cg21413492 | chr6:70990226 | COL9A1.GENEBODY | F | chr6:70992040-70992912 | N_Shore |
| cg22868951 | chr4:129767588 | PHF17.GENEBODY | R | | |
| cg27572053 | chr7:157513908 | PTPRN2.GENEBODY | F | | |
| cg00161124 | chr1:234366259 | SLC35F3.GENEBODY | R | chr1:234367135-234367496 | N_Shore |
| cg00973286 | chr12:6451130 | TNFRSF1A.UTR5 | F | | |
| cg03561416 | chr6:157254584 | ARID1B.GENEBODY | F | | |
| cg07773582 | chr16:4845285 | LOC440335.TSS200 | R | | |
| cg08514594 | chr7:157874020 | PTPRN2.GENEBODY | F | | |
| cg09046979 | chr16:28333134 | SBK1.UTR3 | R | chr16:28331225-28332489 | S_Shore |
| cg10055471 | chr1:27240319 | NR0B2.EXON1 | R | | |
| cg11809826 | chr6:168918901 | SMOC2.GENEBODY | F | chr6:168918596-168918902 | Island |
| cg12286857 | chr7:157530016 | PTPRN2.GENEBODY | F | chr7:157524157-157526741 | S_Shelf |
| cg14495729 | chr7:158283266 | PTPRN2.GENEBODY | R | chr7:158282214-158283382 | Island |
| cg15408722 | chr7:157866922 | PTPRN2.GENEBODY | F | | |
| cg16025118 | chr12:114273678 | RBM19.GENEBODY | F | | |
| cg18789624 | chr7:30516742 | NOD1.UTR5 | F | chr7:30517987-30518610 | N_Shore |
| cg20673767 | chr7:158061805 | PTPRN2.GENEBODY | R | chr7:158060894-158061100 | S_Shore |
| cg20979979 | chr4:141218072 | SCOC.UTR5 | R | | |
| cg22004830 | chr7:157595428 | PTPRN2.GENEBODY | F | chr7:157593887-157594128 | S_Shore |
| cg24438178 | chr11:64120354 | CCDC88B.GENEBODY | F | | |
| cg25023666 | chr21:41585021 | DSCAM.GENEBODY | F | | |
| cg00235442 | chr7:157408294 | PTPRN2.GENEBODY | F | chr7:157409846-157410241 | N_Shore |
| cg04435975 | chr17:46683047 | LOC404266.GENEBODY | F | chr17:46685244-46685449 | N_Shelf |
| cg08462055 | chr15:64944023 | ZNF609.GENEBODY | R | | |
| cg09035925 | chr20:19915769 | RIN2.GENEBODY | R | | |
| cg13889415 | chr9:116943379 | COL27A1.GENEBODY | R | | |
| cg14799696 | chr7:157571491 | PTPRN2.GENEBODY | R | chr7:157568163-157568404 | S_Shelf |
| cg18522715 | chr7:158298958 | PTPRN2.GENEBODY | F | chr7:158301266-158301610 | N_Shelf |
| cg20139338 | chr11:64120656 | CCDC88B.GENEBODY | F | | |
| cg23178546 | chr7:157710554 | PTPRN2.GENEBODY | R | | |
| cg25613641 | chr4:140587143 | MGST2.EXON1 | R | | |
| cg25710298 | chr2:286830 | FAM150B.GENEBODY | F | chr2:287353-290099 | N_Shore |
| cg01103601 | chr2:239288872 | TRAF3IP1.GENEBODY | R | | |
| cg01371493 | chr6:168916145 | SMOC2.GENEBODY | F | chr6:168918596-168918902 | N_Shelf |
| cg01384111 | chr2:11679605 | GREB1.TSS1500 | F | | |
| cg02651776 | chr10:27447805 | MASTL.GENEBODY | F | chr10:27444133-27444494 | S_Shelf |
| cg03021296 | chr7:158060614 | PTPRN2.GENEBODY | F | chr7:158060894-158061100 | N_Shore |
| cg04750536 | chr20:19915857 | RIN2.GENEBODY | R | | |
| cg07399355 | chr1:155163013 | MUC1.TSS1500 | R | chr1:155163434-155163818 | N_Shore |
| cg08271443 | chr16:4845348 | LOC440335.TSS200 | R | | |
| cg08515646 | chr7:157394337 | PTPRN2.GENEBODY | F | | |
| cg11206084 | chr7:157780161 | PTPRN2.GENEBODY | F | | |
| cg13178597 | chr6:153405859 | RGS17.UTR5 | F | | |
| cg14603031 | chr7:2563184 | LFNG.GENEBODY | R | chr7:2563610-2564056 | N_Shore |
| cg16848873 | chr17:46682308 | LOC404266.GENEBODY | F | chr17:46685244-46685449 | N_Shelf |
| cg20146030 | chr2:166650965 | GALNT3.TSS200 | F | chr2:166649909-166650966 | Island |
| cg26808921 | chr6:134373925 | SLC2A12.TSS200 | F | | |
| cg02660277 | chr7:157438616 | PTPRN2.GENEBODY | F | chr7:157440024-157442721 | N_Shore |
| cg03204605 | chr4:7941837 | AFAP1.TSS200 | F | chr4:7940563-7941853 | Island |
| cg03637218 | chr5:115209107 | AP3S1.GENEBODY | F | | |
| cg06233593 | chr16:337645 | AXIN1.UTR3 | F | chr16:335829-336034 | S_Shore |
| cg07267845 | chr7:157345566 | PTPRN2.GENEBODY | R | chr7:157346613-157347069 | N_Shore |
| cg07323947 | chr10:103896068 | PPRC1.GENEBODY | R | chr10:103892659-103893590 | S_Shelf |
| cg08482167 | chr6:144066847 | PHACTR2.GENEBODY | R | | |
| cg08930672 | chr7:157932168 | PTPRN2.GENEBODY | R | | |
| cg09984136 | chr6:37190074 | TMEM217.UTR5 | F | | |
| cg13847963 | chr1:24514220 | IL28RA.TSS1500 | F | chr1:24513540-24514477 | Island |
| cg14653284 | chr2:11679879 | GREB1.UTR5 | R | | |
| cg14800014 | chr5:125800764 | GRAMD3.GENEBODY | R | | |
| cg16872357 | chr7:157529444 | PTPRN2.GENEBODY | R | chr7:157524157-157526741 | S_Shelf |
| cg17086205 | chr7:157543597 | PTPRN2.GENEBODY | R | | |
| cg17284536 | chr6:166891902 | RPS6KA2.GENEBODY | R | | |
| cg18174678 | chr1:42801761 | FOXJ3.TSS1500 | F | chr1:42800265-42801370 | S_Shore |
| cg19476622 | chr18:20714485 | CABLES1.TSS1500 | R | chr18:20714968-20717071 | N_Shore |
| cg19871235 | chr12:71552569 | TSPAN8.TSS1500 | F | | |
| cg23483563 | chr2:163625290 | KCNH7.GENEBODY | F | | |
| cg00332882 | chr20:22401271 | LOC284788.GENEBODY | F | | |
| cg01076708 | chr11:129689897 | TMEM45B.UTR5 | F | chr11:129685737-129686211 | S_Shelf |
| cg02708758 | chr15:74545009 | CCDC33.GENEBODY | R | | |
| cg02978297 | chr1:94057587 | BCAR3.GENEBODY | R | | |
| cg06579626 | chr2:73495921 | FBXO41.GENEBODY | R | chr2:73495920-73496910 | Island |
| cg06998965 | chr5:68710808 | MARVELD2.TSS200 | R | chr5:68710807-68711520 | Island |
| cg07520608 | chr2:23627401 | KLHL29.UTR5 | R | | |
| cg07696757 | chr7:44229001 | GCK.EXON1 | R | | |
| cg08826152 | chr17:15869607 | ADORA2B.GENEBODY | R | | |
| cg09967440 | chr10:127444969 | C10orf137.GENEBODY | F | | |
| cg15016771 | chr2:235403218 | ARL4C.UTR3 | R | chr2:235404502-235406541 | N_Shore |
| cg16666708 | chr19:17797733 | UNC13A.GENEBODY | F | chr19:17797530-17797748 | Island |
| cg18351607 | chr10:124739543 | PSTK.TSS200 | R | chr10:124739792-124740292 | N_Shore |
| cg19502457 | chr14:65409354 | GPX2.EXON1 | R | | |
| cg20464246 | chr1:216774397 | ESRRG.GENEBODY | R | chr1:216774339-216774734 | Island |
| cg00119314 | chr17:48555610 | RSAD1.TSS1500 | F | chr17:48556004-48556509 | N_Shore |
| cg01861131 | chr16:4845708 | LOC440335.GENEBODY | R | | |
| cg02356647 | chr7:158102787 | PTPRN2.GENEBODY | R | chr7:158103336-158103601 | N_Shore |
| cg03391854 | chr7:157961629 | PTPRN2.GENEBODY | F | | |
| cg04476846 | chr5:78791415 | HOMER1.GENEBODY | R | | |
| cg10471944 | chr7:157345246 | PTPRN2.GENEBODY | F | chr7:157346613-157347069 | N_Shore |
| cg10481560 | chr11:1597444 | HCCA2.GENEBODY | R | chr11:1595809-1596143 | S_Shore |
| cg14249078 | chr11:117665480 | DSCAML1.GENEBODY | F | chr11:117666634-117668141 | N_Shore |
| cg17114257 | chr11:72854156 | FCHSD2.TSS1500 | F | chr11:72852176-72854160 | Island |
| cg18645642 | chr1:8631756 | RERE.GENEBODY | R | | |
| cg19948253 | chr11:65318624 | LTBP3.GENEBODY | R | chr11:65321225-65321823 | N_Shelf |
| cg23097035 | chr7:28396164 | CREB5.UTR5 | R | | |
| cg23381267 | chr14:88787370 | KCNK10.GENEBODY | F | chr14:88789600-88790493 | N_Shelf |
| cg25167574 | chr4:79861398 | PAQR3.TSS1500 | F | chr4:79860213-79860745 | S_Shore |
| cg25644224 | chr7:158195876 | PTPRN2.GENEBODY | R | chr7:158198545-158198907 | N_Shelf |
| cg26527638 | chr17:79197398 | AZI1.TSS1500 | R | chr17:79196345-79197419 | Island |
| cg00405069 | chr1:52455390 | RAB3B.UTR5 | R | chr1:52455970-52456453 | N_Shore |
| cg03873826 | chr16:68407311 | SMPD3.UTR5 | F | chr16:68404907-68406273 | S_Shore |
| cg04937416 | chr7:157479272 | PTPRN2.GENEBODY | F | chr7:157476886-157486719 | Island |
| cg04977856 | chr14:89886941 | FOXN3.UTR5 | F | chr14:89882421-89884278 | S_Shelf |
| cg11115622 | chr14:65169958 | PLEKHG3.TSS1500 | R | chr14:65170876-65172007 | N_Shore |
| cg16532223 | chr1:6542083 | PLEKHG5.UTR5 | F | chr1:6545143-6545559 | N_Shelf |
| cg17429772 | chr7:158269409 | PTPRN2.GENEBODY | F | | |
| cg18943033 | chr6:168993309 | SMOC2.GENEBODY | R | | |
| cg21240812 | chr8:143483748 | TSNARE1.UTR5 | F | chr8:143484018-143485051 | N_Shore |
| cg23430388 | chr3:63429828 | SYNPR.GENEBODY | F | | |
| cg25503804 | chr11:46739832 | F2.TSS1500 | R | | |
| cg02694994 | chr7:157612661 | PTPRN2.GENEBODY | R | chr7:157612036-157612316 | S_Shore |
| cg05212138 | chr20:23063119 | CD93.UTR3 | R | | |
| cg07265549 | chr16:27607018 | KIAA0556.GENEBODY | F | | |
| cg09350411 | chr7:157667935 | PTPRN2.GENEBODY | R | chr7:157669879-157670095 | N_Shore |
| cg13794807 | chr4:153871752 | FHDC1.GENEBODY | R | | |
| cg15374582 | chr7:158250524 | PTPRN2.GENEBODY | F | chr7:158247888-158249312 | S_Shore |
| cg25014026 | chr8:73735989 | KCNB2.GENEBODY | F | | |
| cg27373591 | chr18:70208064 | CBLN2.GENEBODY | F | chr18:70208974-70211790 | N_Shore |
| cg01008164 | chr11:4825232 | OR52R1.EXON1 | F | | |
| cg01338378 | chr7:102036355 | PRKRIP1.TSS1500 | F | chr7:102036470-102037017 | N_Shore |
| cg01789980 | chr5:167743144 | WWC1.GENEBODY | R | | |
| cg04682791 | chr6:25313030 | LRRC16A.GENEBODY | F | | |
| cg13917863 | chr11:4945892 | OR51G1.TSS1500 | F | | |
| cg16642299 | chr12:7071383 | MIR200C.TSS1500 | F | | |
| cg21126943 | chr19:42259395 | CEACAM6.TSS200 | F | | |
| cg21316923 | chr2:121983716 | TFCP2L1.GENEBODY | F | chr2:121983326-121983740 | Island |
| cg22054191 | chr19:42637630 | POU2F2.TSS1500 | F | chr19:42637277-42637684 | Island |
| cg22968167 | chr11:4945873 | OR51G1.TSS1500 | F | | |
| cg23447996 | chr9:93564603 | SYK.GENEBODY | R | chr9:93563775-93564546 | S_Shore |
| cg00537335 | chr7:157989340 | PTPRN2.GENEBODY | R | | |
| cg05161074 | chr15:42289885 | PLA2G4E.GENEBODY | F | | |
| cg13068215 | chr1:240286481 | FMN2.GENEBODY | R | | |
| cg16670736 | chr7:157777149 | PTPRN2.GENEBODY | F | | |
| cg21815508 | chr7:157530036 | PTPRN2.GENEBODY | R | chr7:157524157-157526741 | S_Shelf |
| cg21881338 | chr12:125566189 | AACS.GENEBODY | F | chr12:125569828-125570140 | N_Shelf |
| cg22123255 | chr6:30182088 | TRIM26.TSS1500 | F | chr6:30180933-30182187 | Island |
| cg23087306 | chr10:119294055 | EMX2OS.GENEBODY | F | chr10:119293917-119297675 | Island |
| cg00455386 | chr7:140104086 | RAB19.UTR5 | F | | |
| cg00679546 | chr17:60729882 | MRC2.GENEBODY | R | chr17:60729645-60730269 | Island |
| cg01807748 | chr1:94534645 | ABCA4.GENEBODY | R | | |
| cg03758241 | chr9:116929797 | COL27A1.GENEBODY | R | chr9:116930004-116930368 | N_Shore |
| cg13151425 | chr7:2561116 | LFNG.GENEBODY | R | chr7:2558371-2559967 | S_Shore |
| cg14415844 | chr14:91884659 | CCDC88C.TSS1500 | F | chr14:91883672-91885367 | Island |
| cg19513004 | chr6:34206683 | HMGA1.UTR5 | F | chr6:34202567-34206193 | S_Shore |
| cg20217702 | chr3:134094099 | AMOTL2.TSS1500 | F | chr3:134092257-134093741 | S_Shore |
| cg21279806 | chr6:169654719 | THBS2.TSS1500 | R | | |
| cg22017213 | chr4:86851225 | ARHGAP24.GENEBODY | F | | |
| cg22231602 | chr1:241176676 | RGS7.GENEBODY | F | | |
| cg23358740 | chr14:102198157 | C14orf72.GENEBODY | R | | |
| cg27096172 | chr7:158350433 | PTPRN2.GENEBODY | R | chr7:158349387-158350091 | S_Shore |
| cg00590039 | chr6:111888683 | TRAF3IP2.GENEBODY | F | | |
| cg03653855 | chr5:146436093 | PPP2R2B.UTR5 | R | | |
| cg03775459 | chr7:105278448 | ATXN7L1.GENEBODY | R | | |
| cg07602073 | chr1:228595680 | TRIM17.UTR3 | F | chr1:228593811-228594713 | S_Shore |
| cg09682426 | chr6:168901167 | SMOC2.GENEBODY | F | | |
| cg10896862 | chr1:161275561 | MPZ.UTR3 | R | | |
| cg11534680 | chr2:11680020 | GREB1.UTR5 | R | | |
| cg14666310 | chr19:42211479 | CEACAM5.TSS1500 | F | | |
| cg16748524 | chr7:157568392 | PTPRN2.GENEBODY | F | chr7:157568163-157568404 | Island |
| cg23447239 | chr7:157697628 | PTPRN2.GENEBODY | F | chr7:157694387-157694661 | S_Shelf |
| cg24996491 | chr7:157535296 | PTPRN2.GENEBODY | R | | |
| cg01259220 | chr7:914964 | C7orf20.TSS1500 | F | chr7:915753-916644 | N_Shore |
| cg01625621 | chr15:34260433 | AVEN.GENEBODY | R | | |
| cg02671204 | chr16:84076834 | SLC38A8.TSS1500 | F | | |
| cg03512414 | chr20:19915874 | RIN2.GENEBODY | R | | |
| cg04982488 | chr10:116103698 | AFAP1L2.GENEBODY | F | | |
| cg06612016 | chr7:158113184 | PTPRN2.GENEBODY | R | chr7:158109334-158109594 | S_Shelf |
| cg07543823 | chr22:20254968 | RTN4R.GENEBODY | R | chr22:20254889-20255692 | Island |
| cg09985802 | chr10:126234119 | LHPP.GENEBODY | R | | |
| cg12057576 | chr15:78918529 | CHRNB4.GENEBODY | R | | |
| cg12492087 | chr15:42749885 | ZFP106.TSS200 | F | | |
| cg17772793 | chr6:168901202 | SMOC2.GENEBODY | R | | |
| cg19681352 | chr1:27282675 | C1orf172.UTR5 | R | chr1:27286065-27287101 | N_Shelf |
| cg23312425 | chr11:64120623 | CCDC88B.GENEBODY | F | | |
| cg25513853 | chr17:72272298 | DNAI2.UTR5 | F | chr17:72270301-72270512 | S_Shore |
| cg01312997 | chr2:128180646 | PROC.GENEBODY | R | chr2:128180294-128181381 | Island |
| cg02641539 | chr8:105351190 | TM7SF4.TSS1500 | R | | |
| cg04432875 | chr13:113411561 | ATP11A.GENEBODY | R | chr13:113410004-113410362 | S_Shore |
| cg06626786 | chr21:36094858 | NCRNA00160.TSS1500 | R | | |
| cg06977987 | chr11:4902464 | OR51T1.TSS1500 | R | | |
| cg07926178 | chr7:158130463 | PTPRN2.GENEBODY | R | | |
| cg09001440 | chr16:87952767 | CA5A.GENEBODY | F | | |
| cg10926554 | chr6:111888540 | TRAF3IP2.GENEBODY | R | | |
| cg11863717 | chr10:73725041 | CHST3.UTR5 | R | chr10:73723711-73724770 | S_Shore |
| cg12109740 | chr11:72084034 | CLPB.GENEBODY | R | | |
| cg13427810 | chr9:116990636 | COL27A1.GENEBODY | F | | |
| cg23234179 | chr12:88420834 | C12orf50.UTR5 | R | | |
| cg23520574 | chr11:4928572 | OR51A7.TSS200 | R | | |
| cg21756355 | chr7:2233135 | MAD1L1.GENEBODY | F | | |
| cg00794178 | chr3:182897283 | MCF2L2.GENEBODY | F | chr3:182896920-182897381 | Island |
| cg04336561 | chr7:157548939 | PTPRN2.GENEBODY | F | chr7:157550547-157551025 | N_Shore |
| cg05064223 | chr7:157611269 | PTPRN2.GENEBODY | R | chr7:157612036-157612316 | N_Shore |
| cg07047280 | chr21:41516394 | DSCAM.GENEBODY | F | chr21:41516237-41516472 | Island |
| cg07099407 | chr6:43027930 | KLC4.TSS1500 | R | chr6:43026939-43027841 | S_Shore |
| cg07576632 | chr6:168844079 | SMOC2.GENEBODY | R | chr6:168841818-168843100 | S_Shore |
| cg07865091 | chr1:43814306 | MPL.GENEBODY | R | chr1:43814305-43815277 | Island |
| cg12510724 | chr12:6484607 | SCNN1A.GENEBODY | F | | |
| cg13049992 | chr5:125800778 | GRAMD3.GENEBODY | F | | |
| cg17027260 | chr2:109892050 | SH3RF3.GENEBODY | R | | |
| cg19713567 | chr17:37895429 | GRB7.UTR5 | F | | |
| cg21850830 | chr8:124553593 | FBXO32.TSS200 | R | chr8:124552539-124554017 | Island |
| cg22139166 | chr5:124071107 | ZNF608.GENEBODY | F | | |
| cg22211507 | chr3:108564871 | TRAT1.GENEBODY | F | | |
| cg22676000 | chr19:42259081 | CEACAM6.TSS1500 | F | | |
| cg22862420 | chr6:153368919 | RGS17.UTR5 | R | | |
| cg25098281 | chr12:6484713 | SCNN1A.GENEBODY | F | | |
| cg25735076 | chr7:158157881 | PTPRN2.GENEBODY | R | | |
| cg26265390 | chr6:168867606 | SMOC2.GENEBODY | F | | |
| cg26308909 | chr7:2233090 | MAD1L1.GENEBODY | F | | |
| cg12487162 | chr11:12845864 | TEAD1.GENEBODY | R | | |
| cg00467296 | chr11:4928760 | OR51A7.EXON1 | R | | |
| cg00751271 | chr21:36095899 | NCRNA00160.TSS1500 | R | | |
| cg03521982 | chr18:70206471 | CBLN2.GENEBODY | F | chr18:70208974-70211790 | N_Shelf |
| cg05004506 | chr11:4945422 | OR51G1.EXON1 | F | | |
| cg07157169 | chr5:126984994 | CTXN3.EXON1 | F | | |
| cg08867933 | chr15:89150842 | MIR1179.TSS1500 | F | chr15:89147660-89149198 | S_Shore |
| cg12812834 | chr11:4870255 | OR51S1.EXON1 | F | | |
| cg17898069 | chr1:234445489 | SLC35F3.GENEBODY | F | | |
| cg18705776 | chr10:5565829 | CALML3.TSS1500 | R | chr10:5567122-5567828 | N_Shore |
| cg20235871 | chr7:158047359 | PTPRN2.GENEBODY | F | | |
| cg20786131 | chr18:20714496 | CABLES1.TSS1500 | F | chr18:20714968-20717071 | N_Shore |
| cg23181133 | chr19:42300812 | CEACAM3.GENEBODY | F | | |
| cg26169081 | chr10:12648338 | CAMK1D.GENEBODY | R | | |
| cg26430059 | chr14:89770384 | FOXN3.GENEBODY | R | | |
| cg00172803 | chr16:84076941 | SLC38A8.TSS1500 | R | | |
| cg00832555 | chr3:169529716 | LRRC34.GENEBODY | F | chr3:169529875-169530435 | N_Shore |
| cg06656027 | chr12:50016493 | PRPF40B.TSS1500 | R | chr12:50016973-50017466 | N_Shore |
| cg07160163 | chr9:93563778 | SYK.TSS1500 | R | chr9:93563775-93564546 | Island |
| cg08692006 | chr19:4639992 | TNFAIP8L1.TSS200 | F | chr19:4639218-4639750 | S_Shore |
| cg11028923 | chr5:43557644 | PAIP1.TSS1500 | R | chr5:43556419-43557603 | S_Shore |
| cg12364943 | chr10:134108614 | STK32C.GENEBODY | F | | |
| cg14199629 | chr9:84202866 | TLE1.GENEBODY | F | | |
| cg16063747 | chr10:119294173 | EMX2OS.GENEBODY | R | chr10:119293917-119297675 | Island |
| cg20443278 | chr17:77962098 | TBC1D16.GENEBODY | R | chr17:77965805-77966296 | N_Shelf |
| cg21436938 | chr9:116061416 | RNF183.TSS200 | F | | |
| cg23235971 | chr7:157413057 | PTPRN2.GENEBODY | F | chr7:157413901-157414146 | N_Shore |
| cg23361275 | chr8:8749919 | MFHAS1.EXON1 | R | chr8:8748652-8751478 | Island |
| cg24186774 | chr14:63757917 | RHOJ.UTR3 | F | | |
| cg25223634 | chr10:104536695 | C10orf26.GENEBODY | R | | |
| cg00283524 | chr22:44639906 | KIAA1644.UTR3 | F | | |
| cg01582616 | chr7:157595153 | PTPRN2.GENEBODY | R | chr7:157593887-157594128 | S_Shore |
| cg04593696 | chr16:84076900 | SLC38A8.TSS1500 | F | | |
| cg05233289 | chr11:64120728 | CCDC88B.GENEBODY | R | | |
| cg06721096 | chr7:157336864 | PTPRN2.GENEBODY | F | | |
| cg08006727 | chr9:116061614 | RNF183.TSS1500 | R | | |
| cg10173620 | chr15:89658904 | ABHD2.UTR5 | R | | |
| cg14638988 | chr11:62476544 | BSCL2.TSS1500 | F | chr11:62476770-62477481 | N_Shore |
| cg16288617 | chr8:61190272 | CA8.GENEBODY | F | chr8:61193312-61194195 | N_Shelf |
| cg16347213 | chr21:42687940 | FAM3B.TSS1500 | F | chr21:42688746-42689085 | N_Shore |
| cg17801546 | chr1:167090974 | DUSP27.GENEBODY | R | chr1:167090575-167091010 | Island |
| cg19434087 | chr7:157696664 | PTPRN2.GENEBODY | F | chr7:157695025-157695358 | S_Shore |
| cg23632744 | chr11:4674353 | OR51E1.GENEBODY | R | | |
| cg25641451 | chr11:4976263 | OR51A2.EXON1 | F | | |
| cg00648955 | chr2:31456217 | EHD3.TSS1500 | R | chr2:31456740-31457653 | N_Shore |
| cg01759986 | chr20:22401074 | LOC284788.GENEBODY | F | | |
| cg06683080 | chr15:40646549 | PHGR1.GENEBODY | R | chr15:40650055-40651102 | N_Shelf |
| cg08339915 | chr7:157394582 | PTPRN2.GENEBODY | F | | |
| cg08356705 | chr11:68148006 | LRP5.GENEBODY | R | | |
| cg09833475 | chr7:157463804 | PTPRN2.GENEBODY | R | | |
| cg15331500 | chr22:41779351 | TEF.GENEBODY | F | chr22:41777430-41778281 | S_Shore |
| cg19138905 | chr11:4902583 | OR51T1.TSS1500 | R | | |
| cg20083900 | chr7:157624841 | PTPRN2.GENEBODY | F | | |
| cg20984904 | chr7:157529335 | PTPRN2.GENEBODY | R | chr7:157524157-157526741 | S_Shelf |
| cg24939194 | chr7:2563611 | LFNG.GENEBODY | R | chr7:2563610-2564056 | Island |
| cg26434090 | chr11:117665395 | DSCAML1.GENEBODY | R | chr11:117666634-117668141 | N_Shore |
| cg00833703 | chr16:57809842 | KIFC3.GENEBODY | R | | |
| cg03916382 | chr7:157935049 | PTPRN2.GENEBODY | R | chr7:157937614-157937841 | N_Shelf |
| cg04057488 | chr6:168920227 | SMOC2.GENEBODY | R | chr6:168918596-168918902 | S_Shore |
| cg06649856 | chr7:158355003 | PTPRN2.GENEBODY | R | | |
| cg09230014 | chr7:157876369 | PTPRN2.GENEBODY | F | | |
| cg10178498 | chr3:124103021 | KALRN.GENEBODY | F | | |
| cg11374256 | chr8:144888445 | SCRIB.GENEBODY | R | chr8:144887111-144887662 | S_Shore |
| cg13393785 | chr19:8317932 | LASS4.GENEBODY | R | | |
| cg18406801 | chr2:109294820 | LIMS1.GENEBODY | F | | |
| cg22297710 | chr8:79717861 | IL7.TSS200 | F | chr8:79716681-79717403 | S_Shore |
| cg24248505 | chr7:105279790 | ATXN7L1.GENEBODY | R | | |
| cg26396492 | chr20:19915762 | RIN2.GENEBODY | R | | |
| cg09279037 | chr7:158103293 | PTPRN2.GENEBODY | F | chr7:158103336-158103601 | N_Shore |
| cg16284674 | chr10:135123337 | ZNF511.GENEBODY | F | chr10:135123238-135123448 | Island |
| cg01089425 | chr5:10655518 | ANKRD33B.UTR3 | R | | |
| cg03315058 | chr11:62476542 | BSCL2.TSS1500 | F | chr11:62476770-62477481 | N_Shore |
| cg04713531 | chr7:158197288 | PTPRN2.GENEBODY | F | chr7:158198545-158198907 | N_Shore |
| cg07285983 | chr1:174844490 | RABGAP1L.GENEBODY | F | | |
| cg10188797 | chr17:77962048 | TBC1D16.GENEBODY | F | chr17:77965805-77966296 | N_Shelf |
| cg10825530 | chr11:16023708 | SOX6.GENEBODY | R | | |
| cg22209929 | chr3:172394700 | NCEH1.UTR5 | R | | |
| cg23735602 | chr3:178276672 | KCNMB2.UTR5 | R | | |
| cg24153714 | chr10:78057958 | C10orf11.GENEBODY | F | | |
| cg24576995 | chr7:157854008 | PTPRN2.GENEBODY | F | | |
| cg24813588 | chr20:62606499 | SAMD10.UTR3 | R | chr20:62605261-62605463 | S_Shore |
| cg26967186 | chr7:157919649 | PTPRN2.GENEBODY | F | chr7:157918704-157919409 | S_Shore |
| cg01610165 | chr5:124071029 | ZNF608.GENEBODY | R | | |
| cg01083884 | chr13:113641411 | MCF2L.GENEBODY | R | | |
| cg08648047 | chr1:11028561 | C1orf127.GENEBODY | F | | |
| cg12019520 | chr4:8012176 | ABLIM2.GENEBODY | F | | |
| cg15061647 | chr7:158249209 | PTPRN2.GENEBODY | F | chr7:158247888-158249312 | Island |
| cg17658976 | chr7:158111178 | PTPRN2.GENEBODY | F | chr7:158109334-158109594 | S_Shore |
| cg22021832 | chr19:42944717 | CXCL17.GENEBODY | R | | |
| cg24283093 | chr3:14481550 | SLC6A6.UTR5 | F | | |
| cg25291404 | chr20:30252580 | BCL2L1.UTR3 | R | | |
| cg27096807 | chr3:111442336 | PLCXD2.UTR3 | R | | |
| cg10095968 | chr21:45847903 | TRPM2.GENEBODY | R | chr21:45844992-45845693 | S_Shelf |
| cg23631070 | chrX:17652804 | NHS.TSS1500 | R | | |
| cg03079132 | chr16:727212 | RHBDL1.GENEBODY | R | chr16:729438-735815 | N_Shelf |
| cg05195085 | chr7:157951403 | PTPRN2.GENEBODY | F | chr7:157951254-157951800 | Island |
| cg05308556 | chr7:157408770 | PTPRN2.GENEBODY | F | chr7:157409846-157410241 | N_Shore |
| cg06675455 | chr11:4902491 | OR51T1.TSS1500 | R | | |
| cg09958065 | chr10:12648526 | CAMK1D.GENEBODY | R | | |
| cg10249637 | chr7:157655436 | PTPRN2.GENEBODY | F | chr7:157659433-157660322 | N_Shelf |
| cg11161846 | chr1:160990452 | F11R.GENEBODY | R | chr1:160990718-160991225 | N_Shore |
| cg11859489 | chr15:92459058 | SLCO3A1.GENEBODY | F | chr15:92459266-92459678 | N_Shore |
| cg12817840 | chr5:124070989 | ZNF608.GENEBODY | F | | |
| cg16964025 | chr7:157482215 | PTPRN2.GENEBODY | F | chr7:157476886-157486719 | Island |
| cg22531685 | chr7:158264663 | PTPRN2.GENEBODY | R | | |
| cg25541259 | chr2:230576135 | DNER.GENEBODY | R | chr2:230578062-230579981 | N_Shore |
| cg00866953 | chr1:15417078 | KIAA1026.GENEBODY | F | | |
| cg01315919 | chr20:22401404 | LOC284788.TSS200 | F | | |
| cg08705382 | chr1:230322024 | GALNT2.GENEBODY | F | | |
| cg15572489 | chr7:157481091 | PTPRN2.GENEBODY | R | chr7:157476886-157486719 | Island |
| cg21759331 | chr2:209352598 | PTH2R.GENEBODY | R | | |
| cg05439204 | chr7:157854693 | PTPRN2.GENEBODY | F | | |
| cg08240778 | chr11:1643787 | HCCA2.GENEBODY | F | | |
| cg08845722 | chr2:46041801 | PRKCE.GENEBODY | F | | |
| cg09314984 | chr19:5627625 | SAFB.GENEBODY | R | chr19:5622248-5623712 | S_Shelf |
| cg09353345 | chr15:78918560 | CHRNB4.GENEBODY | R | | |
| cg10520790 | chr4:16030846 | PROM1.GENEBODY | R | | |
| cg14157855 | chr1:32226424 | BAI2.UTR5 | R | chr1:32226146-32226535 | Island |
| cg21660130 | chr1:16370243 | CLCNKB.TSS200 | R | | |
| cg22531371 | chr1:155162756 | MUC1.TSS200 | F | chr1:155163434-155163818 | N_Shore |
| cg02690968 | chr11:35683923 | TRIM44.TSS1500 | F | chr11:35684421-35684846 | N_Shore |
| cg03337035 | chr19:42258336 | CEACAM6.TSS1500 | R | | |
| cg03694261 | chr11:122753977 | C11orf63.UTR5 | F | chr11:122753386-122753951 | S_Shore |
| cg04701181 | chr11:1606344 | HCCA2.GENEBODY | F | | |
| cg05327192 | chr11:75133593 | KLHL35.UTR3 | F | chr11:75136407-75136689 | N_Shelf |
| cg06294561 | chr3:141161580 | ZBTB38.GENEBODY | F | | |
| cg12149911 | chr8:61194292 | CA8.TSS1500 | R | chr8:61193312-61194195 | S_Shore |
| cg14651919 | chr17:74075189 | ZACN.TSS200 | R | chr17:74070404-74073530 | S_Shore |
| cg15221831 | chr19:42408464 | ARHGEF1.GENEBODY | F | chr19:42408315-42408535 | Island |
| cg24178676 | chr5:172264981 | ERGIC1.GENEBODY | F | chr5:172261145-172261671 | S_Shelf |
| cg25205321 | chr13:111096339 | COL4A2.GENEBODY | R | | |
| cg00234370 | chr1:36021674 | KIAA0319L.UTR5 | F | chr1:36022680-36023834 | N_Shore |
| cg06716730 | chr17:35851459 | DUSP14.UTR5 | R | chr17:35849290-35850483 | S_Shore |
| cg21521989 | chr7:158269194 | PTPRN2.GENEBODY | F | | |
| cg02855558 | chr7:158107723 | PTPRN2.GENEBODY | F | chr7:158109334-158109594 | N_Shore |
| cg05087455 | chr2:7171869 | RNF144A.GENEBODY | R | chr2:7172296-7172512 | N_Shore |
| cg05295557 | chr14:36988446 | NKX2-1.GENEBODY | F | chr14:36986362-36990576 | Island |
| cg05913555 | chr7:158158760 | PTPRN2.GENEBODY | R | | |
| cg20313969 | chr2:112939562 | FBLN7.GENEBODY | F | chr2:112939287-112939563 | Island |
| cg24611996 | chr10:120353499 | PRLHR.UTR3 | R | chr10:120353692-120355821 | N_Shore |
| cg00735611 | chr17:37895886 | GRB7.UTR5 | R | | |
| cg04854917 | chr10:131378330 | MGMT.GENEBODY | R | | |
| cg09149842 | chr20:31351091 | DNMT3B.UTR5 | R | chr20:31350149-31351263 | Island |
| cg13167816 | chr14:24601808 | FITM1.GENEBODY | F | chr14:24601498-24601700 | S_Shore |
| cg14753321 | chr5:176784914 | RGS14.UTR5 | F | | |
| cg15736062 | chr7:158136485 | PTPRN2.GENEBODY | R | | |
| cg15978565 | chr2:11894464 | LPIN1.UTR5 | R | | |
| cg16809526 | chr16:46917681 | GPT2.TSS1500 | F | chr16:46917849-46918957 | N_Shore |
| cg17019292 | chr5:68710813 | MARVELD2.TSS200 | R | chr5:68710807-68711520 | Island |
| cg27270684 | chr7:55757158 | FKBP9L.TSS200 | R | | |
| cg02711212 | chr15:69854024 | LOC145837.TSS200 | F | | |
| cg04810063 | chr16:88703634 | IL17C.TSS1500 | F | chr16:88706211-88706492 | N_Shelf |
| cg06313349 | chr7:157494424 | PTPRN2.GENEBODY | R | chr7:157494510-157494739 | N_Shore |
| cg11577716 | chr20:20258001 | C20orf26.GENEBODY | F | | |
| cg12144100 | chr7:157890277 | PTPRN2.GENEBODY | R | chr7:157890656-157891557 | N_Shore |
| cg14689490 | chr7:158210489 | PTPRN2.GENEBODY | R | | |
| cg24634333 | chr6:111887834 | TRAF3IP2.GENEBODY | R | | |
| cg07780818 | chr3:133749284 | SLCO2A1.TSS1500 | R | chr3:133747920-133748266 | S_Shore |
| cg09635395 | chr4:146601470 | C4orf51.EXON1 | F | | |
| cg14497491 | chr17:77982477 | TBC1D16.GENEBODY | F | chr17:77983980-77984482 | N_Shore |
| cg15318176 | chr7:157345827 | PTPRN2.GENEBODY | F | chr7:157346613-157347069 | N_Shore |
| cg16792062 | chr11:128458153 | ETS1.TSS1500 | R | | |
| cg19628299 | chr6:168840641 | SMOC2.TSS1500 | F | chr6:168841438-168841699 | N_Shore |
| cg26924822 | chr4:26332763 | RBPJ.GENEBODY | F | | |
| cg00542975 | chr2:122392291 | CLASP1.UTR5 | R | | |
| cg02369113 | chr7:157637560 | PTPRN2.GENEBODY | R | | |
| cg04143211 | chr14:91087927 | TTC7B.GENEBODY | R | chr14:91087475-91087796 | S_Shore |
| cg04489012 | chr1:151812710 | LOC100132111.GENEBODY | F | chr1:151810460-151811684 | S_Shore |
| cg06497408 | chr7:158046838 | PTPRN2.GENEBODY | F | | |
| cg07243405 | chr7:915203 | C7orf20.TSS1500 | R | chr7:915753-916644 | N_Shore |
| cg15490801 | chr14:30054523 | PRKD1.GENEBODY | F | | |
| cg17316096 | chr7:158158218 | PTPRN2.GENEBODY | R | | |
| cg21790851 | chr13:73634643 | KLF5.GENEBODY | F | chr13:73632860-73634370 | S_Shore |
| cg24580076 | chr7:915073 | C7orf20.TSS1500 | F | chr7:915753-916644 | N_Shore |
| cg25727520 | chr2:37576821 | QPCT.GENEBODY | R | | |
| cg26231267 | chr10:12648032 | CAMK1D.GENEBODY | R | | |
| cg03641492 | chr6:168859954 | SMOC2.GENEBODY | R | | |
| cg04162999 | chr11:64120313 | CCDC88B.GENEBODY | R | | |
| cg06400336 | chr8:103136701 | NCALD.TSS200 | R | chr8:103135914-103136775 | Island |
| cg10308629 | chr7:134354803 | BPGM.GENEBODY | R | | |
| cg12813108 | chr7:99719912 | CNPY4.GENEBODY | R | | |
| cg15906675 | chr4:90171420 | GPRIN3.UTR5 | R | | |
| cg18214661 | chr8:17471997 | PDGFRL.GENEBODY | R | | |
| cg00279797 | chrX:109561918 | AMMECR1.UTR5 | R | chrX:109560703-109561534 | S_Shore |
| cg01138652 | chr22:19973978 | ARVCF.GENEBODY | F | chr22:19974274-19974756 | N_Shore |
| cg04457354 | chr6:20447442 | E2F3.GENEBODY | F | | |
| cg05564438 | chr11:64120286 | CCDC88B.GENEBODY | R | | |
| cg14362920 | chr7:157332376 | PTPRN2.UTR3 | R | | |
| cg23392097 | chr11:4704749 | OR51E2.UTR5 | R | | |
| cg26379258 | chr6:111888446 | TRAF3IP2.GENEBODY | R | | |
| cg02753354 | chr19:1074727 | HMHA1.GENEBODY | R | chr19:1074726-1075071 | Island |
| cg04955511 | chr7:14801527 | DGKB.GENEBODY | F | | |
| cg05093113 | chr20:49200925 | MIR645.TSS1500 | F | | |
| cg07756135 | chr4:86748944 | ARHGAP24.GENEBODY | F | | |
| cg09832443 | chr7:28612680 | CREB5.GENEBODY | F | | |
| cg13464117 | chr7:157348161 | PTPRN2.GENEBODY | R | chr7:157346613-157347069 | S_Shore |
| cg14141549 | chr7:157919408 | PTPRN2.GENEBODY | F | chr7:157918704-157919409 | Island |
| cg15353612 | chr4:52904965 | SGCB.TSS1500 | F | chr4:52904454-52904912 | S_Shore |
| cg21191737 | chr9:15306337 | TTC39B.GENEBODY | F | chr9:15306722-15307386 | N_Shore |
| cg23574281 | chr2:47716 | FAM110C.TSS1500 | R | chr2:45510-46559 | S_Shore |
| cg02479744 | chr6:16303089 | ATXN1.UTR3 | R | | |
| cg02735652 | chr1:32226179 | BAI2.UTR5 | R | chr1:32226146-32226535 | Island |
| cg05175762 | chr7:157987088 | PTPRN2.GENEBODY | F | | |
| cg09614629 | chr7:157985057 | PTPRN2.GENEBODY | R | chr7:157980786-157981462 | S_Shelf |
| cg10754395 | chr7:158109694 | PTPRN2.GENEBODY | F | chr7:158110569-158110881 | N_Shore |
| cg11980090 | chr6:30181771 | TRIM26.TSS1500 | R | chr6:30180933-30182187 | Island |
| cg12419766 | chr7:157620445 | PTPRN2.GENEBODY | R | chr7:157618856-157619302 | S_Shore |
| cg12882572 | chr17:35851204 | DUSP14.UTR5 | R | chr17:35849290-35850483 | S_Shore |
| cg22356339 | chr12:103352454 | ASCL1.EXON1 | R | chr12:103351579-103352695 | Island |
| cg26085804 | chr11:72854242 | FCHSD2.TSS1500 | F | chr11:72852176-72854160 | S_Shore |
| cg00816406 | chr1:240286626 | FMN2.GENEBODY | F | | |
| cg01718785 | chr7:157916279 | PTPRN2.GENEBODY | F | chr7:157918704-157919409 | N_Shelf |
| cg02601893 | chr22:43523016 | BIK.GENEBODY | F | | |
| cg13689591 | chr3:124239629 | KALRN.GENEBODY | F | | |
| cg14575854 | chr17:37896175 | GRB7.UTR5 | F | | |
| cg20208009 | chr22:19974048 | ARVCF.GENEBODY | F | chr22:19974274-19974756 | N_Shore |
| cg26572392 | chr10:24496943 | KIAA1217.UTR5 | R | | |
| cg26572992 | chr19:18876246 | CRTC1.GENEBODY | F | chr19:18873019-18873349 | S_Shelf |
| cg26593067 | chr7:157446092 | PTPRN2.GENEBODY | R | chr7:157448240-157448566 | N_Shelf |
| cg01760027 | chrX:49832257 | CLCN5.GENEBODY | R | | |
| cg13700912 | chr7:1968348 | MAD1L1.GENEBODY | R | chr7:1967664-1967994 | S_Shore |
| cg14817241 | chr19:4851575 | PLIN3.GENEBODY | F | chr19:4851962-4852248 | N_Shore |
| cg16872071 | chr9:135997155 | RALGDS.GENEBODY | F | chr9:135995969-135996954 | S_Shore |
| cg20867746 | chr11:4871813 | OR51S1.TSS1500 | R | | |
| cg21376148 | chr17:10276260 | MYH13.UTR5 | R | | |
| cg01352090 | chr16:4103533 | ADCY9.GENEBODY | F | | |
| cg01808739 | chr1:93912802 | FNBP1L.TSS1500 | F | chr1:93913636-93914524 | N_Shore |
| cg07219844 | chr10:135123764 | ZNF511.GENEBODY | F | chr10:135121991-135122669 | S_Shore |
| cg09526758 | chr12:16758047 | LMO3.UTR5 | F | | |
| cg14507792 | chr9:95574913 | ANKRD19.GENEBODY | R | chr9:95569429-95572255 | S_Shelf |
| cg16609181 | chr19:1083007 | HMHA1.GENEBODY | R | chr19:1082576-1083430 | Island |
| cg22337375 | chr7:157351042 | PTPRN2.GENEBODY | R | chr7:157352995-157353232 | N_Shore |
| cg00926318 | chr20:25177340 | ENTPD6.UTR5 | R | chr20:25176027-25176620 | S_Shore |
| cg02051806 | chr12:86650107 | MGAT4C.UTR5 | R | | |
| cg10709461 | chr20:22402729 | LOC284788.TSS1500 | F | | |
| cg12379775 | chr20:62669791 | NCRNA00176.GENEBODY | F | chr20:62669304-62670730 | Island |
| cg13265740 | chr6:139349120 | C6orf115.TSS1500 | R | chr6:139349955-139350740 | N_Shore |
| cg14192174 | chr2:29204117 | FAM179A.TSS200 | F | | |
| cg18856478 | chr1:43814358 | MPL.GENEBODY | R | chr1:43814305-43815277 | Island |
| cg20697464 | chr2:218793716 | TNS1.UTR5 | R | | |
| cg23637314 | chr1:216774226 | ESRRG.GENEBODY | F | chr1:216774339-216774734 | N_Shore |
| cg00008819 | chr4:3257791 | C4orf44.UTR3 | F | chr4:3254957-3255175 | S_Shelf |
| cg00115178 | chr10:12648176 | CAMK1D.GENEBODY | R | | |
| cg12538248 | chr8:61194072 | CA8.TSS200 | R | chr8:61193312-61194195 | Island |
| cg12806381 | chr1:27114177 | PIGV.TSS1500 | F | chr1:27114118-27114692 | Island |
| cg21292473 | chr1:181683239 | CACNA1E.GENEBODY | R | | |
| cg11315900 | chr7:157605970 | PTPRN2.GENEBODY | R | | |
| cg22581896 | chr7:4779225 | FOXK1.GENEBODY | R | | |
| cg25124966 | chr11:129723987 | TMEM45B.GENEBODY | R | | |
| cg25718402 | chr17:7165909 | CLDN7.TSS200 | F | chr17:7164285-7166245 | Island |
| cg02925076 | chr7:158251297 | PTPRN2.GENEBODY | F | chr7:158247888-158249312 | S_Shore |
| cg09176256 | chr10:119293967 | EMX2OS.GENEBODY | F | chr10:119293917-119297675 | Island |
| cg18099070 | chr7:157845265 | PTPRN2.GENEBODY | R | | |
| cg26425555 | chr5:16616459 | FAM134B.GENEBODY | F | chr5:16616509-16617428 | N_Shore |
| cg26532826 | chr22:31003127 | TCN2.TSS200 | R | | |
| cg26147269 | chr3:13520256 | HDAC11.TSS1500 | F | chr3:13520912-13521319 | N_Shore |
| cg14632140 | chr12:16758112 | LMO3.UTR5 | R | | |
| cg19091386 | chr7:157435297 | PTPRN2.GENEBODY | F | | |
| cg04657700 | chr2:223535084 | MOGAT1.TSS1500 | F | chr2:223536122-223536565 | N_Shore |
| cg12132731 | chr1:205313575 | KLHDC8A.UTR5 | F | chr1:205312493-205313576 | Island |
| cg17956079 | chr11:73358572 | PLEKHB1.TSS200 | R | | |
| cg01731355 | chr7:158103582 | PTPRN2.GENEBODY | F | chr7:158103336-158103601 | Island |
| cg05116002 | chr3:58151255 | FLNB.GENEBODY | R | | |
| cg05518803 | chr10:124739553 | PSTK.TSS200 | R | chr10:124739792-124740292 | N_Shore |
| cg12233379 | chr1:151812524 | LOC100132111.GENEBODY | F | chr1:151812253-151812525 | Island |
| cg09565319 | chr6:168963731 | SMOC2.GENEBODY | R | | |
| cg03721064 | chr7:50535834 | DDC.GENEBODY | R | chr7:50535740-50535953 | Island |
| cg18011899 | chr7:158124253 | PTPRN2.GENEBODY | F | | |
| cg23967873 | chr10:72977537 | UNC5B.GENEBODY | R | chr10:72977123-72977774 | Island |
| cg27430662 | chr1:109656708 | KIAA1324.UTR5 | R | chr1:109656095-109656611 | S_Shore |
| cg00870849 | chr10:94003008 | CPEB3.UTR5 | F | chr10:93999131-94000015 | S_Shelf |
| cg05765011 | chr16:4103225 | ADCY9.GENEBODY | F | | |
| cg18104606 | chr6:70990361 | COL9A1.GENEBODY | R | chr6:70992040-70992912 | N_Shore |
| cg10724947 | chr15:62359485 | C2CD4A.UTR5 | F | chr15:62359827-62360995 | N_Shore |
| cg12271047 | chr7:157387106 | PTPRN2.GENEBODY | R | chr7:157387053-157387322 | Island |
| cg17833037 | chr12:123259751 | CCDC62.GENEBODY | R | | |
| cg20354580 | chr3:128713146 | KIAA1257.TSS200 | R | chr3:128712404-128713058 | S_Shore |
| cg09508022 | chr6:168971135 | SMOC2.GENEBODY | F | chr6:168972516-168974109 | N_Shore |
| cg15744134 | chr15:32933835 | SCG5.TSS200 | R | | |
| cg20587332 | chr8:40736768 | ZMAT4.UTR5 | R | | |
| cg21276549 | chr13:102568345 | FGF14.GENEBODY | F | chr13:102568425-102569495 | N_Shore |
| cg21390575 | chr4:185747721 | ACSL1.TSS1500 | R | chr4:185746557-185747827 | Island |
| cg03905369 | chr2:120824615 | EPB41L5.GENEBODY | F | | |
| cg09918657 | chr16:14397207 | MIR193B.TSS1500 | R | chr16:14395604-14397075 | S_Shore |
| cg05640721 | chr15:90437833 | AP3S2.TSS1500 | F | chr15:90437025-90437602 | S_Shore |
| cg06059849 | chr1:109656930 | KIAA1324.EXON1 | F | chr1:109656095-109656611 | S_Shore |
| cg12609243 | chr3:54155240 | CACNA2D3.TSS1500 | R | chr3:54155239-54157025 | Island |
| cg19744528 | chr7:157553346 | PTPRN2.GENEBODY | F | chr7:157550547-157551025 | S_Shelf |
| cg22740141 | chr12:129454194 | GLT1D1.GENEBODY | F | | |
| cg14981313 | chr1:42847055 | RIMKLA.GENEBODY | R | chr1:42845978-42846988 | S_Shore |
| cg06023257 | chr11:78129396 | GAB2.TSS1500 | F | chr11:78127965-78129400 | Island |
| cg06233018 | chr21:41552656 | DSCAM.GENEBODY | F | chr21:41554618-41554959 | N_Shore |
| cg09306186 | chr16:73081722 | ZFHX3.EXON1 | F | chr16:73081590-73082565 | Island |
| cg10545682 | chr1:151512625 | TUFT1.TSS200 | F | chr1:151512661-151513199 | N_Shore |
| cg10769178 | chr7:157876300 | PTPRN2.GENEBODY | R | | |
| cg24440468 | chr7:73184642 | CLDN3.TSS200 | R | chr7:73183379-73185115 | Island |
| cg26824709 | chr5:159740383 | CCNJL.TSS1500 | R | chr5:159738781-159739913 | S_Shore |
| cg01461199 | chr8:121822473 | SNTB1.GENEBODY | F | chr8:121823534-121824720 | N_Shore |
| cg03721293 | chr16:90014378 | DEF8.TSS1500 | F | chr16:90014250-90014613 | Island |
| cg00336946 | chr13:101186274 | A2LD1.TSS1500 | F | chr13:101184396-101184848 | S_Shore |
| cg02375832 | chr11:62437615 | C11orf48.UTR5 | R | chr11:62439047-62439803 | N_Shore |
| cg16027376 | chr8:144653975 | C8orf73.GENEBODY | F | chr8:144653774-144653976 | Island |
| cg09565670 | chr19:39368408 | RINL.UTR5 | R | | |
| cg07915206 | chr15:34260555 | AVEN.GENEBODY | R | | |
| cg11445764 | chr5:147773164 | FBXO38.UTR5 | R | | |
| cg24918790 | chr22:20785148 | SCARF2.GENEBODY | F | chr22:20783466-20786201 | Island |

TABLE 3
Top 1000 methylation sites associated with the SCLC-N subtype

| Methylation | Position | | | | Relation to |
|---|---|---|---|---|---|
| Site | (GRCh 37) | Gene | Strand | CpG Island Name | CpG Island |
| cg20728514 | chr22:44568423 | PARVG.TSS1500 | R | | |
| cg04506569 | chr2:234837977 | TRPM8.GENEBODY | F | | |
| cg12688942 | chr1:19680353 | CAPZB.GENEBODY | R | | |
| cg07213482 | chr4:89660237 | FAM13A.GENEBODY | R | | |
| cg11201256 | chr1:184403226 | C1orf21.UTR5 | R | | |
| cg18966819 | chr7:4784325 | FOXK1.GENEBODY | R | chr7:4784820-4785058 | N_Shore |
| cg08946713 | chr2:191844998 | STAT1.GENEBODY | F | | |
| cg27119612 | chr15:57491001 | TCF12.GENEBODY | R | | |
| cg04921578 | chr13:101143257 | PCCA.GENEBODY | F | | |
| cg01441025 | chr8:103136942 | NCALD.EXON1 | F | chr8:103135914-103136775 | S_Shore |
| cg16896647 | chr9:93563776 | SYK.TSS1500 | R | chr9:93563775-93564546 | Island |
| cg18883682 | chr7:36655667 | AOAH.GENEBODY | R | | |
| cg00624799 | chr15:90605618 | ZNF710.UTR5 | R | | |
| cg02390801 | chr15:74528565 | CCDC33.TSS200 | F | | |
| cg16311186 | chr7:140114137 | RAB19.GENEBODY | F | | |
| cg19747960 | chr8:11269117 | C8orf12.GENEBODY | R | | |
| cg27336068 | chr10:1336103 | ADARB2.GENEBODY | R | | |
| cg21732383 | chr6:169654692 | THBS2.TSS1500 | F | | |
| cg25029446 | chr7:157495282 | PTPRN2.GENEBODY | F | chr7:157494510-157494739 | S_Shore |
| cg00911446 | chr1:7520079 | CAMTA1.GENEBODY | F | | |
| cg12761282 | chr20:2276571 | TGM3.TSS200 | R | | |
| cg07696006 | chr15:74528563 | CCDC33.TSS200 | F | | |
| cg11035519 | chr11:130051458 | ST14.GENEBODY | F | | |
| cg11281961 | chr2:97498902 | CNNM3.UTR3 | R | | |
| cg06221087 | chr1:164659666 | PBX1.GENEBODY | F | | |
| cg02716952 | chr7:148504681 | EZH2.UTR3 | R | | |
| cg25148025 | chr1:164681267 | PBX1.GENEBODY | F | | |
| cg24216770 | chr20:35091947 | DLGAP4.GENEBODY | F | chr20:35089448-35089998 | S_Shore |
| cg19404354 | chr1:202093479 | GPR37L1.GENEBODY | F | | |
| cg19637113 | chr15:74528522 | CCDC33.TSS200 | F | | |
| cg19188359 | chr20:2312099 | TGM3.GENEBODY | R | chr20:2308778-2309042 | S_Shelf |
| cg19779605 | chr11:4703553 | OR51E2.GENEBODY | R | | |
| cg20448594 | chr7:73940352 | GTF2IRD1.GENEBODY | R | | |
| cg04198210 | chr15:74528606 | CCDC33.TSS200 | F | | |
| cg06070324 | chr15:75322058 | PPCDC.GENEBODY | R | | |
| cg20632462 | chr11:20882850 | NELL1.GENEBODY | F | | |
| cg16696476 | chr17:8191344 | RANGRF.TSS1500 | R | chr17:8191864-8192495 | N_Shore |
| cg22797644 | chr6:34206491 | HMGA1.UTR5 | F | chr6:34202567-34206193 | S_Shore |
| cg02079584 | chr15:74528719 | CCDC33.EXON1 | F | | |
| cg02902261 | chr7:72991150 | TBL2.GENEBODY | R | chr7:72992434-72993397 | N_Shore |
| cg08989212 | chr21:36096051 | NCRNA00160.TSS200 | R | | |
| cg00827176 | chr8:21885702 | NPM2.GENEBODY | R | chr8:21882245-21882883 | S_Shelf |
| cg01511231 | chr1:1109676 | TTLL10.UTR5 | F | chr1:1109314-1110145 | Island |
| cg07143635 | chr16:11681487 | LITAF.TSS200 | F | chr16:11680008-11681275 | S_Shore |
| cg20573218 | chr3:181409863 | SOX2OT.GENEBODY | F | chr3:181413014-181414022 | N_Shelf |
| cg20825710 | chr11:1620352 | HCCA2.GENEBODY | R | | |
| cg25809301 | chr3:71179811 | FOXP1.GENEBODY | F | | |
| cg04188351 | chr11:33744823 | CD59.UTR5 | R | | |
| cg06287775 | chr7:4784174 | FOXK1.GENEBODY | R | chr7:4784820-4785058 | N_Shore |
| cg07530194 | chr17:78808450 | RPTOR.GENEBODY | R | | |
| cg01431993 | chr2:182542864 | NEUROD1.GENEBODY | R | | |
| cg08570034 | chr5:175227702 | CPLX2.UTR5 | F | chr5:175223609-175224679 | S_Shelf |
| cg01863682 | chr2:182545771 | NEUROD1.TSS1500 | R | chr2:182547873-182549177 | N_Shelf |
| cg22705918 | chr2:25518238 | DNMT3A.GENEBODY | F | | |
| cg22719871 | chr11:66105148 | BRMS1.UTR3 | R | chr11:66102024-66102279 | S_Shelf |
| cg22792988 | chr5:76376904 | SNORA47.TSS1500 | F | chr5:76372882-76373720 | S_Shelf |
| cg01782132 | chr16:28936054 | RABEP2.GENEBODY | R | chr16:28936053-28937036 | Island |
| cg24424217 | chr10:135123421 | ZNF511.GENEBODY | F | chr10:135123238-135123448 | Island |
| cg00161124 | chr1:234366259 | SLC35F3.GENEBODY | R | chr1:234367135-234367496 | N_Shore |
| cg02544257 | chr16:70510663 | FUK.GENEBODY | R | chr16:70507026-70507246 | S_Shelf |
| cg07773582 | chr16:4845285 | LOC440335.TSS200 | R | | |
| cg08335767 | chr6:34206495 | HMGA1.UTR5 | F | chr6:34202567-34206193 | S_Shore |
| cg07399355 | chr1:155163013 | MUC1.TSS1500 | R | chr1:155163434-155163818 | N_Shore |
| cg08271443 | chr16:4845348 | LOC440335.TSS200 | R | | |
| cg18780243 | chr13:32487268 | EEF1DP3.GENEBODY | R | | |
| cg25430878 | chr10:1257999 | ADARB2.GENEBODY | R | | |
| cg09984136 | chr6:37190074 | TMEM217.UTR5 | F | | |
| cg17284536 | chr6:166891902 | RPS6KA2.GENEBODY | R | | |
| cg15553397 | chr1:76695186 | ST6GALNAC3.GENEBODY | R | | |
| cg01861131 | chr16:4845708 | LOC440335.GENEBODY | R | | |
| cg05234415 | chr19:38743744 | PPP1R14A.GENEBODY | R | chr19:38746638-38747379 | N_Shelf |
| cg17114257 | chr11:72854156 | FCHSD2.TSS1500 | F | chr11:72852176-72854160 | Island |
| cg26527638 | chr17:79197398 | AZI1.TSS1500 | R | chr17:79196345-79197419 | Island |
| cg16177633 | chr1:232172585 | DISC1.UTR3 | R | | |
| cg16532223 | chr1:6542083 | PLEKHG5.UTR5 | F | chr1:6545143-6545559 | N_Shelf |
| cg21240812 | chr8:143483748 | TSNARE1.UTR5 | F | chr8:143484018-143485051 | N_Shore |
| cg25014026 | chr8:73735989 | KCNB2.GENEBODY | F | | |
| cg26775087 | chr3:15382808 | SH3BP5.EXON1 | R | | |
| cg01008405 | chr5:139039026 | CXXC5.UTR5 | F | chr5:139040819-139041028 | N_Shore |
| cg08082862 | chr16:67807580 | RANBP10.GENEBODY | F | | |
| cg22123255 | chr6:30182088 | TRIM26.TSS1500 | F | chr6:30180933-30182187 | Island |
| cg19513004 | chr6:34206683 | HMGA1.UTR5 | F | chr6:34202567-34206193 | S_Shore |
| cg21279806 | chr6:169654719 | THBS2.TSS1500 | R | | |
| cg22017213 | chr4:86851225 | ARHGAP24.GENEBODY | F | | |
| cg22539431 | chr7:127423253 | SND1.GENEBODY | R | | |
| cg02671204 | chr16:84076834 | SLC38A8.TSS1500 | F | | |
| cg07543823 | chr22:20254968 | RTN4R.GENEBODY | R | chr22:20254889-20255692 | Island |
| cg19681352 | chr1:27282675 | C1orf172.UTR5 | R | chr1:27286065-27287101 | N_Shelf |
| cg06626786 | chr21:36094858 | NCRNA00160.TSS1500 | R | | |
| cg11528849 | chr2:182542901 | NEUROD1.GENEBODY | R | | |
| cg02964356 | chr12:49883750 | SPATS2.GENEBODY | R | | |
| cg11686009 | chr20:2305747 | TGM3.GENEBODY | R | chr20:2308778-2309042 | N_Shelf |
| cg16826777 | chr17:43376882 | MAP3K14.UTR5 | F | | |
| cg18705776 | chr10:5565829 | CALML3.TSS1500 | R | chr10:5567122-5567828 | N_Shore |
| cg20444218 | chr11:73024037 | ARHGEF17.GENEBODY | F | chr11:73018452-73020563 | S_Shelf |
| cg05214130 | chr12:123945990 | SNRNP35.GENEBODY | F | chr12:123942639-123943001 | S_Shelf |
| cg07160163 | chr9:93563778 | SYK.TSS1500 | R | chr9:93563775-93564546 | Island |
| cg12581598 | chr1:202537837 | PPP1R12B.GENEBODY | F | | |
| cg12551557 | chr12:32308125 | BICD1.GENEBODY | R | | |
| cg16347213 | chr21:42687940 | FAM3B.TSS1500 | F | chr21:42688746-42689085 | N_Shore |
| cg22797514 | chr1:44016023 | PTPRF.GENEBODY | F | | |
| cg24575128 | chr3:52502445 | NISCH.GENEBODY | R | | |
| cg06683080 | chr15:40646549 | PHGR1.GENEBODY | R | chr15:40650055-40651102 | N_Shelf |
| cg08356705 | chr11:68148006 | LRP5.GENEBODY | R | | |
| cg14583225 | chr17:40074010 | ACLY.UTR5 | R | chr17:40074291-40075380 | N_Shore |
| cg21602520 | chr18:60985380 | BCL2.GENEBODY | F | chr18:60985503-60985741 | N_Shore |
| cg00833703 | chr16:57809842 | KIFC3.GENEBODY | R | | |
| cg05310309 | chr2:109257429 | LIMS1.UTR5 | F | | |
| cg24938166 | chr2:70941682 | ADD2.UTR5 | R | | |
| cg13685679 | chr17:79231688 | SLC38A10.GENEBODY | F | chr17:79235486-79235751 | N_Shelf |
| cg08102294 | chr3:13063918 | IQSEC1.GENEBODY | F | | |
| cg11747695 | chr13:111164966 | COL4A2.UTR3 | F | chr13:111164284-111164547 | S_Shore |
| cg15691129 | chrX:68725815 | FAM155B.EXON1 | R | chrX:68723731-68726051 | Island |
| cg00254714 | chr20:2309923 | TGM3.GENEBODY | R | chr20:2308778-2309042 | S_Shore |
| cg02624246 | chr12:7073264 | MIR141.GENEBODY | F | | |
| cg06164804 | chr2:240044389 | HDAC4.GENEBODY | R | chr2:240046200-240046736 | N_Shore |
| cg10999000 | chr13:111180025 | RAB20.GENEBODY | F | | |
| cg19404444 | chr1:2164602 | SKI.GENEBODY | R | chr1:2158212-2161173 | S_Shelf |
| cg25003275 | chr4:7940128 | AFAP1.UTR5 | F | chr4:7940563-7941853 | N_Shore |
| cg08515811 | chr17:77921489 | TBC1D16.GENEBODY | R | chr17:77922721-77922973 | N_Shore |
| cg09314984 | chr19:5627625 | SAFB.GENEBODY | R | chr19:5622248-5623712 | S_Shelf |
| cg16717192 | chr7:4265800 | SDK1.GENEBODY | R | | |
| cg16537859 | chrX:70440209 | BCYRN1.GENEBODY | F | chrX:70444046-70444270 | N_Shelf |
| cg22544881 | chr7:130712346 | FLJ43663.GENEBODY | R | | |
| cg00806680 | chr6:41561085 | FOXP4.GENEBODY | F | chr6:41561221-41561473 | N_Shore |
| cg04204246 | chr6:112537238 | LAMA4.GENEBODY | R | | |
| cg11938455 | chr6:15622032 | DTNBP1.GENEBODY | R | | |
| cg13417891 | chr17:35836996 | TADA2A.GENEBODY | F | | |
| cg14672994 | chr17:48503057 | ACSF2.TSS1500 | R | chr17:48503056-48503887 | Island |
| cg23540651 | chr1:89490034 | GBP3.TSS1500 | R | | |
| cg27470486 | chr17:40073688 | ACLY.UTR5 | F | chr17:40074291-40075380 | N_Shore |
| cg17959824 | chr11:70391706 | SHANK2.GENEBODY | F | | |
| cg21781706 | chr1:6438350 | ACOT7.GENEBODY | F | | |
| cg06520307 | chr7:149417640 | KRBA1.GENEBODY | F | chr7:149416662-149416974 | S_Shore |
| cg07790176 | chr13:92601293 | GPC5.GENEBODY | R | | |
| cg17602451 | chr18:60985645 | BCL2.GENEBODY | R | chr18:60985503-60985741 | Island |
| cg25871713 | chr3:37823949 | ITGA9.GENEBODY | R | | |
| cg01961752 | chr1:1109012 | TTLL10.TSS1500 | R | chr1:1109314-1110145 | N_Shore |
| cg03486974 | chr16:2199702 | RAB26.GENEBODY | R | chr16:2198455-2199129 | S_Shore |
| cg03767531 | chr2:182545572 | NEUROD1.TSS200 | F | chr2:182547873-182549177 | N_Shelf |
| cg18470891 | chr12:2986957 | FOXM1.TSS1500 | R | chr12:2985978-2986639 | S_Shore |
| cg02364642 | chr12:58005758 | GEFT.EXON1 | R | chr12:58003880-58004249 | S_Shore |
| cg02643580 | chr12:95671146 | VEZT.GENEBODY | R | | |
| cg02836529 | chr2:182544413 | NEUROD1.UTR5 | R | chr2:182547873-182549177 | N_Shelf |
| cg03641492 | chr6:168859954 | SMOC2.GENEBODY | R | | |
| cg04048740 | chr2:236442999 | AGAP1.GENEBODY | F | chr2:236443815-236444025 | N_Shore |
| cg10198932 | chr21:43136287 | C21orf129.TSS1500 | F | | |
| cg10308629 | chr7:134354803 | BPGM.GENEBODY | R | | |
| cg17093877 | chr17:72206412 | MGC16275.GENEBODY | F | chr17:72208474-72208902 | N_Shelf |
| cg04457354 | chr6:20447442 | E2F3.GENEBODY | F | | |
| cg22193393 | chr15:42290845 | PLA2G4E.GENEBODY | R | | |
| cg04531425 | chr2:182322841 | ITGA4.GENEBODY | F | chr2:182321761-182323029 | Island |
| cg06979020 | chr16:69660388 | NFAT5.UTR5 | R | | |
| cg20978600 | chr3:40566157 | ZNF621.TSS1500 | R | chr3:40566129-40566689 | Island |
| cg23574281 | chr2:47716 | FAM110C.TSS1500 | R | chr2:45510-46559 | S_Shore |
| cg00459232 | chr12:6309025 | CD9.TSS1500 | R | chr12:6309152-6310392 | N_Shore |
| cg05722741 | chr6:168897588 | SMOC2.GENEBODY | F | | |
| cg08134671 | chr19:2542837 | GNG7.UTR5 | R | chr19:2543638-2543878 | N_Shore |
| cg11980090 | chr6:30181771 | TRIM26.TSS1500 | R | chr6:30180933-30182187 | Island |
| cg18822036 | chr8:131371659 | ASAP1.GENEBODY | R | chr8:131370087-131371317 | S_Shore |
| cg20535931 | chr7:97615658 | OCM2.GENEBODY | F | | |
| cg27567922 | chr3:195480636 | MUC4.GENEBODY | F | chr3:195477711-195477983 | S_Shelf |
| cg13374528 | chr7:129414627 | MIR96.TSS200 | R | chr7:129418286-129422509 | N_Shelf |
| cg00690082 | chr17:40438511 | STAT5A.TSS1500 | R | chr17:40440188-40441014 | N_Shore |
| cg00929655 | chr15:92459909 | SLCO3A1.GENEBODY | R | chr15:92459266-92459678 | S_Shore |
| cg01983725 | chr1:227161799 | CABC1.GENEBODY | F | chr1:227163739-227164237 | N_Shore |
| cg02058628 | chr1:2164542 | SKI.GENEBODY | F | chr1:2158212-2161173 | S_Shelf |
| cg07219844 | chr10:135123764 | ZNF511.GENEBODY | F | chr10:135121991-135122669 | S_Shore |
| cg02051924 | chr16:11681482 | LITAF.TSS200 | R | chr16:11680008-11681275 | S_Shore |
| cg24728949 | chr5:106820169 | EFNA5.GENEBODY | F | | |
| cg02276086 | chr2:163175335 | IFIH1.TSS1500 | F | | |
| cg26392737 | chr8:21916853 | EPB49.EXON1 | F | chr8:21914275-21914525 | S_Shelf |
| cg00797278 | chr3:32442928 | CMTM7.GENEBODY | F | chr3:32443152-32443455 | N_Shore |
| cg01435515 | chr1:151255742 | ZNF687.UTR5 | R | chr1:151253658-151255769 | Island |
| cg02710090 | chr1:207225128 | PFKFB2.TSS1500 | F | chr1:207226281-207226842 | N_Shore |
| cg04633683 | chr16:29837056 | MVP.UTR5 | R | | |
| cg04905719 | chr6:134617250 | SGK1.GENEBODY | F | | |
| cg09035736 | chr22:38599166 | MAFF.UTR5 | F | chr22:38597785-38598988 | S_Shore |
| cg09556527 | chr1:1239259 | ACAP3.GENEBODY | F | chr1:1242400-1245185 | N_Shelf |
| cg05379127 | chr19:12885939 | HOOK2.GENEBODY | F | chr19:12885756-12886519 | Island |
| cg26147269 | chr3:13520256 | HDAC11.TSS1500 | F | chr3:13520912-13521319 | N_Shore |
| cg17107112 | chr6:34021546 | GRM4.GENEBODY | R | chr6:34024201-34024457 | N_Shelf |
| cg09890699 | chr1:54821853 | SSBP3.GENEBODY | R | chr1:54821990-54822333 | N_Shore |
| cg14882082 | chr14:100845427 | WDR25.UTR5 | F | chr14:100842339-100842931 | S_Shelf |
| cg16071713 | chr21:36412456 | C21orf96.TSS1500 | F | | |
| cg23674344 | chr17:48503163 | ACSF2.TSS1500 | F | chr17:48503056-48503887 | Island |
| cg00489219 | chr3:40566141 | ZNF621.TSS1500 | F | chr3:40566129-40566689 | Island |
| cg08230118 | chr6:30181847 | TRIM26.TSS1500 | R | chr6:30180933-30182187 | Island |
| cg10451262 | chr8:124284176 | ZHX1.UTR5 | R | chr8:124286209-124287666 | N_Shelf |
| cg16522557 | chr13:111159111 | COL4A2.GENEBODY | F | | |
| cg20427144 | chr7:5518918 | FBXL18.UTR3 | F | chr7:5521368-5521615 | N_Shelf |
| cg20806345 | chr2:182543706 | NEUROD1.UTR5 | F | | |
| cg02185402 | chr20:400361 | RBCK1.GENEBODY | F | chr20:400244-400469 | Island |
| cg07908874 | chr10:135123006 | ZNF511.GENEBODY | R | chr10:135122851-135123109 | Island |
| cg21913981 | chr12:120151706 | MIR1178.TSS200 | R | | |
| cg26363039 | chr12:1609549 | LOC100292680.TSS200 | R | | |
| cg00870849 | chr10:94003008 | CPEB3.UTR5 | F | chr10:93999131-94000015 | S_Shelf |
| cg08521073 | chr12:117314052 | HRK.UTR3 | R | chr12:117316389-117317611 | N_Shelf |
| cg12218991 | chr4:152042934 | SH3D19.UTR3 | F | | |
| cg14014950 | chr3:64125334 | PRICKLE2.GENEBODY | R | | |
| cg14220262 | chr1:1114963 | TTLL10.GENEBODY | F | chr1:1115574-1116006 | N_Shore |
| cg20417351 | chr17:424358 | VPS53.GENEBODY | R | | |
| cg01154445 | chr2:206548171 | NRP2.GENEBODY | F | chr2:206549599-206551316 | N_Shore |
| cg04569165 | chrX:153688677 | PLXNA3.GENEBODY | F | chrX:153688592-153688860 | Island |
| cg04957757 | chr16:70463218 | ST3GAL2.UTR5 | R | | |
| cg07660671 | chr8:10586967 | SOX7.GENEBODY | F | chr8:10587304-10591109 | N_Shore |
| cg09354317 | chr3:52869307 | MUSTN1.TSS200 | F | | |
| cg11484283 | chr4:159816181 | FNIP2.GENEBODY | F | | |
| cg19563107 | chr14:53418879 | FERMT2.TSS1500 | R | chr14:53417108-53418339 | S_Shore |
| cg22110158 | chr11:130036542 | ST14.GENEBODY | F | | |
| cg22369288 | chr6:138538817 | PBOV1.EXON1 | R | | |
| cg02954284 | chr22:21367447 | MGC16703.GENEBODY | R | chr22:21368197-21368771 | N_Shore |
| cg08059112 | chr19:2294887 | LINGO3.UTR5 | R | chr19:2294886-2295101 | Island |
| cg13859860 | chr10:51569878 | NCOA4.UTR5 | F | chr10:51572198-51572620 | N_Shelf |
| cg14666908 | chr6:31937758 | STK19.TSS1500 | R | chr6:31939730-31940559 | N_Shore |
| cg16404364 | chr7:1006746 | COX19.UTR3 | F | chr7:1007159-1007558 | N_Shore |
| cg19711579 | chr2:182545562 | NEUROD1.TSS200 | F | chr2:182547873-182549177 | N_Shelf |
| cg22976218 | chr2:182544321 | NEUROD1.UTR5 | R | chr2:182547873-182549177 | N_Shelf |
| cg03569876 | chr5:148908014 | CSNK1A1.GENEBODY | R | | |
| cg03674563 | chr11:422260 | ANO9.GENEBODY | R | chr11:420265-420975 | S_Shore |
| cg06374811 | chr6:22099667 | FLJ22536.GENEBODY | R | | |
| cg19359218 | chr6:30181936 | TRIM26.TSS1500 | R | chr6:30180933-30182187 | Island |
| cg21163701 | chr8:85165845 | RALYL.UTR5 | F | | |
| cg26743132 | chr13:111107905 | COL4A2.GENEBODY | F | chr13:111109139-111109652 | N_Shore |
| cg19754622 | chr10:134045578 | STK32C.GENEBODY | F | chr10:134041499-134041784 | S_Shelf |
| cg03769629 | chr6:30181591 | TRIM26.TSS1500 | R | chr6:30180933-30182187 | Island |
| cg22222281 | chr12:6308758 | CD9.TSS1500 | R | chr12:6309152-6310392 | N_Shore |
| cg25903363 | chr8:21972479 | HR.UTR3 | R | | |
| cg00321901 | chr8:10512459 | RP1L1.EXON1 | R | | |
| cg03695433 | chr17:71332907 | SDK2.UTR3 | F | | |
| cg24408763 | chr6:43446221 | TJAP1.UTR5 | R | | |
| cg24603926 | chr12:120151679 | MIR1178.TSS200 | R | | |
| cg27098470 | chr22:42828411 | NFAM1.TSS200 | R | | |
| cg02102889 | chr4:139164723 | SLC7A11.TSS1500 | R | | |
| cg04176122 | chr11:118779835 | BCL9L.GENEBODY | F | chr11:118781060-118781732 | N_Shore |
| cg15409237 | chr19:51524249 | KLK10.TSS1500 | F | chr19:51522004-51522803 | S_Shore |
| cg17401435 | chr11:63887426 | MACROD1.GENEBODY | F | chr11:63887355-63887600 | Island |
| cg19008877 | chr10:78157854 | C10orf11.GENEBODY | R | | |
| cg11286977 | chr6:30181713 | TRIM26.TSS1500 | R | chr6:30180933-30182187 | Island |
| cg01027553 | chr7:4037843 | SDK1.GENEBODY | F | | |
| cg01155325 | chr15:42719757 | ZFP106.GENEBODY | R | | |
| cg06714480 | chr2:182544307 | NEUROD1.UTR5 | R | chr2:182547873-182549177 | N_Shelf |
| cg07181952 | chr1:1239999 | ACAP3.GENEBODY | F | chr1:1242400-1245185 | N_Shelf |
| cg09306186 | chr16:73081722 | ZFHX3.EXON1 | F | chr16:73081590-73082565 | Island |
| cg20709008 | chr2:182545934 | NEUROD1.TSS1500 | F | chr2:182547873-182549177 | N_Shore |
| cg25356639 | chr3:71349304 | FOXP1.UTR5 | F | | |
| cg01788682 | chr3:15382723 | SH3BP5.UTR5 | F | | |
| cg07109923 | chr20:35091317 | DLGAP4.GENEBODY | R | chr20:35089448-35089998 | S_Shore |
| cg13538006 | chr9:95897313 | NINJ1.TSS1500 | R | chr9:95896007-95897016 | S_Shore |
| cg13818276 | chr22:32437865 | SLC5A1.TSS1500 | R | chr22:32439046-32439442 | N_Shore |
| cg08530036 | chr1:19496406 | UBR4.GENEBODY | R | | |
| cg09388414 | chr7:129414651 | MIR96.TSS200 | R | chr7:129418286-129422509 | N_Shelf |
| cg18124764 | chr7:101558698 | CUX1.GENEBODY | F | chr7:101559402-101559625 | N_Shore |
| cg23406864 | chr22:21053952 | TMEM191A.TSS1500 | R | chr22:21056258-21058793 | N_Shelf |
| cg23731742 | chr1:879958 | NOC2L.UTR3 | R | chr1:875730-878363 | S_Shore |
| cg00735329 | chr18:74732603 | MBP.GENEBODY | R | | |
| cg04757345 | chr4:128918835 | C4orf29.GENEBODY | F | | |
| cg19681793 | chr6:169651645 | THBS2.UTR5 | R | chr6:169648546-169649047 | S_Shelf |
| cg22332321 | chr20:62510260 | TPD52L2.GENEBODY | R | chr20:62509163-62509369 | S_Shore |
| cg22817352 | chr1:204653629 | LRRN2.UTR5 | F | chr1:204654132-204654885 | N_Shore |
| cg03723730 | chr6:34031694 | GRM4.GENEBODY | F | | |
| cg20660385 | chr19:51526587 | KLK11.GENEBODY | R | chr19:51522004-51522803 | S_Shelf |
| cg20708173 | chr4:7940426 | AFAP1.UTR5 | R | chr4:7940563-7941853 | N_Shore |
| cg23218957 | chr14:59109856 | DACT1.GENEBODY | R | chr14:59113071-59113640 | N_Shelf |
| cg14660446 | chr19:48015621 | NAPA.GENEBODY | F | chr19:48017717-48018782 | N_Shelf |
| cg19296671 | chr11:128562297 | FLI1.TSS200 | R | chr11:128562671-128565011 | N_Shore |
| cg02921634 | chr21:43109546 | NCRNA00111.GENEBODY | F | chr21:43109349-43109687 | Island |
| cg03365751 | chr22:42828386 | NFAM1.EXON1 | F | | |
| cg06136185 | chr17:79139087 | LOC388428.TSS1500 | R | chr17:79139501-79140028 | N_Shore |
| cg06963053 | chr2:182545533 | NEUROD1.TSS200 | F | chr2:182547873-182549177 | N_Shelf |
| cg10806639 | chr13:33780078 | STARD13.EXON1 | R | | |
| cg20970369 | chr1:111744108 | DENND2D.TSS1500 | F | chr1:111746337-111747303 | N_Shelf |
| cg21244086 | chr15:45479462 | SHF.GENEBODY | F | chr15:45479509-45480498 | N_Shore |
| cg10159951 | chr11:35441881 | SLC1A2.TSS1500 | F | chr11:35440375-35441882 | Island |
| cg10614445 | chr2:25526696 | DNMT3A.GENEBODY | F | | |
| cg22486214 | chr17:78930466 | RPTOR.GENEBODY | F | | |
| cg25043538 | chr11:63887437 | MACROD1.GENEBODY | R | chr11:63887355-63887600 | Island |
| cg01670896 | chr8:1933522 | KBTBD11.UTR5 | F | | |
| cg02959939 | chr21:47813025 | PCNT.GENEBODY | F | chr21:47813251-47813462 | N_Shore |
| cg07533617 | chr11:117624926 | DSCAML1.GENEBODY | R | | |
| cg10143807 | chr1:155050924 | EFNA3.TSS1500 | F | chr1:155051155-155055291 | N_Shore |
| cg14765206 | chr10:101744219 | DNMBP.UTR5 | R | | |
| cg21798544 | chr11:924646 | AP2A2.TSS1500 | R | chr11:925183-926083 | N_Shore |
| cg26142132 | chr17:74463585 | AANAT.TSS200 | R | | |
| cg03368643 | chr6:30181600 | TRIM26.TSS1500 | R | chr6:30180933-30182187 | Island |
| cg07827420 | chr17:75371764 | SEPT9.UTR5 | F | chr17:75368688-75370506 | S_Shore |
| cg15935860 | chr1:201282499 | PKP1.GENEBODY | F | | |
| cg26813796 | chr1:225806065 | ENAH.GENEBODY | R | | |
| cg19778462 | chr1:41976072 | HIVEP3.UTR3 | F | | |
| cg22149419 | chr17:27253138 | PHF12.GENEBODY | R | | |
| cg12710538 | chr17:27490340 | MYO18A.GENEBODY | F | chr17:27493203-27493880 | N_Shelf |
| cg13250203 | chr11:1565798 | HCCA2.GENEBODY | R | chr11:1567940-1568907 | N_Shelf |
| cg26245202 | chr1:205602864 | ELK4.TSS1500 | F | chr1:205600493-205601432 | S_Shore |
| cg01474011 | chr2:225435131 | CUL3.GENEBODY | F | | |
| cg08549332 | chr7:101558461 | CUX1.GENEBODY | F | chr7:101559402-101559625 | N_Shore |
| cg16935790 | chr17:77043246 | C1QTNF1.GENEBODY | R | chr17:77043741-77044489 | N_Shore |
| cg21285555 | chr8:52771466 | PCMTD1.GENEBODY | R | | |
| cg04956949 | chr7:73119262 | STX1A.GENEBODY | F | chr7:73118500-73118749 | S_Shore |
| cg08519905 | chr12:6308774 | CD9.TSS1500 | R | chr12:6309152-6310392 | N_Shore |
| cg09560636 | chr5:43631579 | NNT.GENEBODY | R | | |
| cg12400725 | chr1:29240533 | EPB41.UTR5 | R | chr1:29240768-29241969 | N_Shore |
| cg19397765 | chr6:30181764 | TRIM26.TSS1500 | R | chr6:30180933-30182187 | Island |
| cg21585162 | chr11:128566387 | FLI1.GENEBODY | F | chr11:128562671-128565011 | S_Shore |
| cg25004427 | chr5:326523 | AHRR.GENEBODY | R | chr5:320788-323010 | S_Shelf |
| cg08821738 | chr17:77043179 | C1QTNF1.GENEBODY | F | chr17:77043741-77044489 | N_Shore |
| cg14570838 | chr11:128562353 | FLI1.TSS1500 | F | chr11:128562671-128565011 | N_Shore |
| cg17397159 | chr4:720144 | PCGF3.UTR5 | F | chr4:716281-716977 | S_Shelf |
| cg18601229 | chr15:42192297 | EHD4.UTR3 | R | chr15:42192913-42193255 | N_Shore |
| cg00162859 | chr8:131268256 | ASAP1.GENEBODY | F | | |
| cg08162476 | chr3:13064001 | IQSEC1.GENEBODY | R | | |
| cg02893260 | chr13:30071603 | MTUS2.GENEBODY | R | | |
| cg03508204 | chr2:129057540 | HS6ST1.GENEBODY | F | | |
| cg04176557 | chr11:75902270 | WNT11.GENEBODY | R | chr11:75905717-75905918 | N_Shelf |
| cg05230392 | chr17:55976522 | CUEDC1.UTR5 | F | | |
| cg05912053 | chr15:60919520 | RORA.GENEBODY | R | | |
| cg12758867 | chr11:65354231 | EHBP1L1.GENEBODY | F | chr11:65352231-65353134 | S_Shore |
| cg13889934 | chr4:86851399 | ARHGAP24.GENEBODY | F | | |
| cg14454073 | chr14:50159754 | KLHDC1.TSS200 | F | chr14:50159667-50160424 | Island |
| cg14475480 | chr17:79655469 | HGS.GENEBODY | R | chr17:79655162-79655515 | Island |
| cg18041814 | chr6:36802896 | CPNE5.GENEBODY | F | chr6:36806881-36807217 | N_Shelf |
| cg20735041 | chr3:52869266 | MUSTN1.TSS200 | R | | |
| cg04658856 | chr6:16242468 | GMPR.GENEBODY | R | chr6:16238589-16239231 | S_Shelf |
| cg05004108 | chr1:85931167 | DDAH1.TSS1500 | F | chr1:85929940-85931168 | Island |
| cg14986845 | chr6:30182240 | TRIM26.TSS1500 | R | chr6:30180933-30182187 | S_Shore |
| cg15742605 | chr1:879383 | SAMD11.GENEBODY | F | chr1:875730-878363 | S_Shore |
| cg20894936 | chr11:1630266 | HCCA2.GENEBODY | F | | |
| cg23417096 | chr8:134250016 | NDRG1.UTR3 | R | | |
| cg27104413 | chr3:101044447 | SENP7.UTR3 | R | | |
| cg21648069 | chr12:1157718 | ERC1.GENEBODY | F | | |
| cg00192882 | chr17:19291120 | MFAP4.TSS1500 | R | | |
| cg02838877 | chr13:97873877 | MBNL2.TSS1500 | R | | |
| cg03841136 | chr21:43136835 | NCRNA00112.GENEBODY | R | | |
| cg09591406 | chr17:3833873 | ATP2A3.GENEBODY | R | | |
| cg09901529 | chr10:75437967 | AGAP5.GENEBODY | F | chr10:75434361-75434584 | S_Shelf |
| cg25595636 | chr11:61278148 | LRRC10B.EXON1 | F | chr11:61275825-61277329 | S_Shore |
| cg02716826 | chr9:33447032 | SUGT1P1.GENEBODY | R | chr9:33447446-33447824 | N_Shore |
| cg07017242 | chr7:128047441 | IMPDH1.GENEBODY | F | chr7:128045542-128046298 | S_Shore |
| cg16944941 | chr11:70391346 | SHANK2.GENEBODY | F | | |
| cg18264519 | chr2:97214296 | ARID5A.GENEBODY | F | chr2:97215788-97216225 | N_Shore |
| cg19626737 | chr15:68633792 | ITGA11.GENEBODY | F | | |
| cg21745110 | chr11:19798742 | NAV2.GENEBODY | R | chr11:19798537-19798949 | Island |
| cg04098194 | chr1:1241087 | ACAP3.GENEBODY | R | chr1:1242400-1245185 | N_Shore |
| cg07524505 | chr11:44785843 | TSPAN18.TSS200 | R | | |
| cg11808936 | chr4:7894203 | AFAP1.UTR5 | F | | |
| cg11503687 | chr5:138205434 | CTNNA1.GENEBODY | F | | |
| cg12116749 | chr10:21245178 | NEBL.GENEBODY | R | | |
| cg02047211 | chr17:78932697 | RPTOR.GENEBODY | R | chr17:78935043-78935307 | N_Shelf |
| cg02839802 | chr22:38963969 | DMC1.GENEBODY | F | chr22:38965733-38966196 | N_Shore |
| cg04278162 | chr6:30182172 | TRIM26.TSS1500 | F | chr6:30180933-30182187 | Island |
| cg19918429 | chr3:122399781 | PARP14.EXON1 | F | chr3:122399655-122400190 | Island |
| cg00156118 | chr2:85473750 | TCF7L1.GENEBODY | R | | |
| cg04545079 | chr16:67599924 | CTCF.UTR5 | R | chr16:67595868-67597554 | S_Shelf |
| cg08692676 | chr6:134299594 | TBPL1.UTR5 | R | | |
| cg09342325 | chr2:220371699 | GMPPA.UTR3 | F | | |
| cg11314042 | chr6:143380966 | AIG1.TSS1500 | F | chr6:143381444-143381918 | N_Shore |
| cg14326805 | chr9:71737756 | TJP2.UTR5 | F | chr9:71736074-71737367 | S_Shore |
| cg15018282 | chr6:30181877 | TRIM26.TSS1500 | R | chr6:30180933-30182187 | Island |
| cg25887619 | chr7:101558435 | CUX1.GENEBODY | R | chr7:101559402-101559625 | N_Shore |
| cg03850117 | chr16:87894334 | SLC7A5.GENEBODY | R | chr16:87894359-87894566 | N_Shore |
| cg15509065 | chr19:36346314 | KIRREL2.TSS1500 | F | chr19:36347044-36348101 | N_Shore |
| cg16105461 | chr11:61485853 | DAGLA.UTR5 | F | chr11:61488132-61488357 | N_Shelf |
| cg16933706 | chr6:30181934 | TRIM26.TSS1500 | R | chr6:30180933-30182187 | Island |
| cg19139618 | chr17:46504791 | SKAP1.GENEBODY | R | chr17:46507344-46507778 | N_Shelf |
| cg21804531 | chr14:90419290 | C14orf143.GENEBODY | F | chr14:90420900-90421175 | N_Shore |
| cg23735214 | chr11:77595033 | INTS4.GENEBODY | F | | |
| cg26329692 | chr17:73126757 | NT5C.GENEBODY | R | chr17:73127196-73127995 | N_Shore |
| cg03478739 | chr2:175712381 | CHN1.GENEBODY | F | | |
| cg04828792 | chr21:42733433 | MX2.TSS1500 | F | | |
| cg08258650 | chr11:35441900 | SLC1A2.TSS1500 | F | chr11:35440375-35441882 | S_Shore |
| cg08348709 | chr17:37764802 | NEUROD2.TSS1500 | F | chr17:37761691-37763551 | S_Shore |
| cg18085176 | chr4:7939940 | AFAP1.UTR5 | F | chr4:7940563-7941853 | N_Shore |
| cg00785029 | chr21:43491755 | UMODL1.GENEBODY | R | | |
| cg06804344 | chr3:128778699 | GP9.TSS1500 | F | | |
| cg06865992 | chr17:37764503 | NEUROD2.TSS1500 | R | chr17:37761691-37763551 | S_Shore |
| cg08427782 | chr6:166880989 | RPS6KA2.GENEBODY | F | | |
| cg14059959 | chr13:111144447 | COL4A2.GENEBODY | R | | |
| cg18498622 | chrX:151810246 | GABRQ.GENEBODY | F | chrX:151806233-151806947 | S_Shelf |
| cg20192387 | chr6:166856056 | RPS6KA2.GENEBODY | R | | |
| cg21917090 | chr4:77816250 | ANKRD56.UTR3 | R | chr4:77818015-77819600 | N_Shore |
| cg22359606 | chr2:182545492 | NEUROD1.TSS200 | F | chr2:182547873-182549177 | N_Shelf |
| cg24554839 | chr1:166124903 | MIR921.TSS1500 | F | | |
| cg25788549 | chr7:150786044 | AGAP3.GENEBODY | F | chr7:150783480-150785040 | S_Shore |
| cg08671326 | chr3:194967958 | C3orf21.GENEBODY | F | | |
| cg13920460 | chr9:71735701 | TJP2.TSS1500 | R | chr9:71736074-71737367 | N_Shore |
| cg19482025 | chr11:128562245 | FLI1.TSS200 | F | chr11:128562671-128565011 | N_Shore |
| cg04907173 | chr11:65060386 | POLA2.GENEBODY | F | | |
| cg05124770 | chr22:24578799 | SUSD2.GENEBODY | F | chr22:24581632-24582127 | N_Shelf |
| cg22666115 | chr6:30182186 | TRIM26.TSS1500 | F | chr6:30180933-30182187 | Island |
| cg23641264 | chr8:143748641 | JRK.UTR5 | F | chr8:143750759-143751448 | N_Shelf |
| cg03979241 | chr8:21916824 | EPB49.EXON1 | F | chr8:21914275-21914525 | S_Shelf |
| cg07651914 | chr7:100881986 | CLDN15.TSS1500 | F | | |
| cg13974838 | chr9:98221619 | PTCH1.GENEBODY | F | | |
| cg15612436 | chr22:43897902 | MPPED1.GENEBODY | R | | |
| cg16222790 | chr8:144521374 | ZC3H3.GENEBODY | R | chr8:144521358-144521608 | Island |
| cg17066349 | chr12:120130646 | CIT.GENEBODY | F | chr12:120127973-120128291 | S_Shelf |
| cg25320780 | chr1:110933345 | SLC16A4.UTR5 | F | | |
| cg01877565 | chr7:129414746 | MIR183.GENEBODY | F | chr7:129418286-129422509 | N_Shelf |
| cg04305228 | chr22:18272333 | MICAL3.UTR3 | R | chr22:18275679-18275922 | N_Shelf |
| cg06433132 | chr19:39662346 | PAK4.GENEBODY | R | chr19:39663600-39663815 | N_Shore |
| cg10832281 | chr11:35499741 | PAMR1.GENEBODY | F | | |
| cg14627862 | chr6:169641232 | THBS2.GENEBODY | F | chr6:169641803-169642020 | N_Shore |
| cg19695266 | chr1:1241672 | ACAP3.GENEBODY | R | chr1:1242400-1245185 | N_Shore |
| cg02619205 | chr9:117157157 | AKNA.TSS1500 | F | chr9:117160139-117161064 | N_Shelf |
| cg03539717 | chr19:13065086 | GADD45GIP1.GENEBODY | R | chr19:13067585-13068152 | N_Shelf |
| cg04007028 | chr17:36735584 | SRCIN1.GENEBODY | R | chr17:36734659-36735246 | S_Shore |
| cg05499054 | chr8:75148806 | JPH1.UTR3 | R | | |
| cg11401784 | chr1:16092264 | FBLIM1.GENEBODY | R | | |
| cg20491406 | chr1:201693818 | NAV1.GENEBODY | R | | |
| cg23377852 | chr5:1339812 | CLPTM1L.GENEBODY | F | chr5:1339662-1340082 | Island |
| cg00912746 | chr1:33350709 | HPCA.TSS1500 | F | chr1:33351686-33352397 | N_Shore |
| cg01557332 | chr6:169641045 | THBS2.GENEBODY | R | chr6:169641803-169642020 | N_Shore |
| cg08160706 | chr16:11876111 | ZC3H7A.GENEBODY | F | | |
| cg10716524 | chr11:474509 | PTDSS2.GENEBODY | R | chr11:472781-473065 | S_Shore |
| cg11410436 | chr16:52581609 | TOX3.UTR5 | F | chr16:52581293-52581610 | Island |
| cg19432362 | chr13:46426263 | SIAH3.TSS1500 | F | | |
| cg03107310 | chr10:86094757 | FAM190B.UTR5 | F | | |
| cg06097994 | chr17:77043171 | C1QTNF1.GENEBODY | F | chr17:77043741-77044489 | N_Shore |
| cg13290371 | chr7:5518071 | FBXL18.UTR3 | R | chr7:5521368-5521615 | N_Shelf |
| cg13948413 | chr1:236687580 | LGALS8.UTR5 | F | chr1:236687071-236687608 | Island |
| cg15027294 | chr12:1609537 | LOC100292680.TSS200 | R | | |
| cg15962197 | chr1:41704561 | SCMH1.UTR5 | R | chr1:41706957-41708327 | N_Shelf |
| cg17097293 | chr19:3655825 | PIP5K1C.GENEBODY | F | chr19:3659641-3659949 | N_Shelf |
| cg21464975 | chr17:60828260 | MARCH10.GENEBODY | R | | |
| cg21832801 | chr11:107579390 | SLN.UTR5 | R | | |
| cg22019158 | chr1:27926683 | AHDC1.UTR5 | R | chr1:27929705-27930866 | N_Shelf |
| cg05212460 | chr7:2149814 | MAD1L1.GENEBODY | R | | |
| cg11856932 | chr12:53447059 | TENC1.UTR5 | F | chr12:53448008-53448406 | N_Shore |
| cg13160058 | chr8:26243215 | BNIP3L.GENEBODY | F | chr8:26240290-26241017 | S_Shelf |
| cg15118447 | chr6:159220343 | EZR.GENEBODY | F | | |
| cg22962041 | chr17:49235043 | NME1-NME2.GENEBODY | R | chr17:49230686-49231247 | S_Shelf |
| cg25619638 | chr2:220310232 | SPEG.GENEBODY | F | chr2:220309637-220309893 | S_Shore |
| cg11076275 | chr1:207069654 | IL24.TSS1500 | F | | |
| cg13808641 | chr9:96006533 | WNK2.GENEBODY | R | chr9:96009844-96010101 | N_Shelf |
| cg20666533 | chr13:67714028 | PCDH9.GENEBODY | F | | |
| cg22311015 | chr2:241630042 | AQP12A.TSS1500 | F | | |
| cg05712010 | chr1:230895283 | CAPN9.GENEBODY | R | | |
| cg06488150 | chr7:6476003 | DAGLB.GENEBODY | F | | |
| cg08969344 | chr6:34031597 | GRM4.GENEBODY | R | | |
| cg09537448 | chr7:129414567 | MIR96.GENEBODY | F | chr7:129418286-129422509 | N_Shelf |
| cg18171392 | chr22:24938255 | C22orf13.UTR3 | F | | |
| cg20951539 | chr6:36367819 | PXT1.GENEBODY | R | | |
| cg21635706 | chr15:69432565 | MIR548H4.GENEBODY | R | | |
| cg23375717 | chr11:20883318 | NELL1.GENEBODY | F | | |
| cg25670451 | chr12:6487677 | SCNN1A.TSS1500 | R | | |
| cg09901733 | chr17:1731852 | RPA1.TSS1500 | R | chr17:1732834-1733612 | N_Shore |
| cg16640855 | chr2:182543233 | NEUROD1.GENEBODY | R | | |
| cg22047469 | chr1:1110107 | TTLL10.UTR5 | R | chr1:1109314-1110145 | Island |
| cg22468751 | chr6:136824489 | MAP7.GENEBODY | F | | |
| cg12299554 | chr15:94840953 | MCTP2.TSS1500 | R | | |
| cg22169990 | chr7:150786051 | AGAP3.GENEBODY | F | chr7:150783480-150785040 | S_Shore |
| cg23286081 | chr10:125775778 | CHST15.GENEBODY | R | | |
| cg00005437 | chr17:18010740 | DRG2.UTR3 | F | | |
| cg01132785 | chr4:528175 | PIGG.GENEBODY | F | chr4:529166-529556 | N_Shore |
| cg02124184 | chr5:72950691 | RGNEF.UTR5 | F | | |
| cg05940240 | chr22:41762302 | TEF.TSS1500 | F | | |
| cg07096883 | chr7:149979234 | ACTR3C.GENEBODY | R | | |
| cg10206969 | chr7:5518392 | FBXL18.UTR3 | F | chr7:5521368-5521615 | N_Shelf |
| cg12941836 | chr3:52869288 | MUSTN1.TSS200 | F | | |
| cg19254163 | chr11:60623782 | GPR44.TSS1500 | R | chr11:60619924-60621111 | S_Shelf |
| cg27412310 | chr11:75146692 | GDPD5.GENEBODY | R | | |
| cg07414392 | chr9:71735648 | TJP2.TSS1500 | R | chr9:71736074-71737367 | N_Shore |
| cg13568213 | chr9:136387235 | TMEM8C.GENEBODY | F | | |
| cg02482603 | chr1:174843754 | RABGAP1L.TSS1500 | F | | |
| cg05380808 | chr1:210820877 | HHAT.GENEBODY | R | | |
| cg05593510 | chr5:176249224 | UNC5A.GENEBODY | R | chr5:176244559-176245284 | S_Shelf |
| cg07505680 | chr8:1933651 | KBTBD11.UTR5 | F | | |
| cg08539179 | chr1:227915948 | LOC100130093.TSS1500 | R | chr1:227916425-227916736 | N_Shore |
| cg10068572 | chr10:35687029 | CCNY.GENEBODY | F | | |
| cg11065015 | chr20:327170 | NRSN2.TSS200 | R | chr20:327376-328977 | N_Shore |
| cg24092939 | chr12:49181056 | ADCY6.UTR5 | F | chr12:49182421-49182658 | N_Shore |
| cg24321971 | chr11:27406919 | LGR4.GENEBODY | R | | |
| cg21887193 | chr7:150786082 | AGAP3.GENEBODY | F | chr7:150783480-150785040 | S_Shore |
| cg02108198 | chr7:99527876 | GJC3.TSS1500 | F | | |
| cg14755690 | chr7:128868265 | AHCYL2.GENEBODY | R | chr7:128864320-128865655 | S_Shelf |
| cg17476271 | chr11:128562408 | FLI1.TSS1500 | F | chr11:128562671-128565011 | N_Shore |
| cg24522085 | chr2:169556419 | LASS6.GENEBODY | F | | |
| cg04785838 | chr6:30181742 | TRIM26.TSS1500 | F | chr6:30180933-30182187 | Island |
| cg08529481 | chr1:36832173 | STK40.UTR5 | F | | |
| cg17838427 | chr1:204653956 | LRRN2.UTR5 | F | chr1:204654132-204654885 | N_Shore |
| cg06011067 | chr6:110677913 | C6orf186.GENEBODY | R | chr6:110678854-110679108 | N_Shore |
| cg07562888 | chr10:80883720 | ZMIZ1.UTR5 | F | | |
| cg10598711 | chr5:72470395 | TMEM174.UTR3 | R | | |
| cg12417176 | chr15:55788778 | DYX1C1.GENEBODY | F | chr15:55790301-55790754 | N_Shore |
| cg21738316 | chrX:153688775 | PLXNA3.GENEBODY | F | chrX:153688592-153688860 | Island |
| cg22626525 | chr1:20008900 | TMCO4.UTR3 | F | chr1:20005510-20005865 | S_Shelf |
| cg23956760 | chr19:19178708 | SLC25A42.UTR5 | R | chr19:19174262-19174995 | S_Shelf |
| cg24352878 | chr1:8772712 | RERE.UTR5 | R | | |
| cg27654966 | chr7:101558400 | CUX1.GENEBODY | R | chr7:101559402-101559625 | N_Shore |
| cg13714797 | chr9:7103479 | KDM4C.GENEBODY | R | | |
| cg23971638 | chr12:741507 | NINJ2.GENEBODY | R | | |
| cg24204017 | chr3:57999988 | FLNB.GENEBODY | F | | |
| cg26017492 | chr6:166891729 | RPS6KA2.GENEBODY | F | | |
| cg00369376 | chr2:182542510 | NEUROD1.UTR3 | F | | |
| cg02864954 | chr6:154535553 | IPCEF1.GENEBODY | R | | |
| cg03505501 | chr11:115371789 | CADM1.GENEBODY | R | chr11:115373574-115375881 | N_Shore |
| cg05161682 | chr13:111107167 | COL4A2.GENEBODY | R | chr13:111109139-111109652 | N_Shore |
| cg09427605 | chr4:146740968 | ZNF827.GENEBODY | R | | |
| cg13396830 | chr6:30181581 | TRIM26.TSS1500 | R | chr6:30180933-30182187 | Island |
| cg16427420 | chr17:74526161 | CYGB.GENEBODY | F | | |
| cg26504422 | chr19:44277951 | KCNN4.GENEBODY | R | chr19:44278273-44278777 | N_Shore |
| cg10156302 | chr15:64202084 | DAPK2.GENEBODY | F | | |
| cg13866253 | chr11:77093001 | PAK1.GENEBODY | R | | |
| cg14465207 | chr2:10951250 | PDIA6.GENEBODY | F | chr2:10952402-10953384 | N_Shore |
| cg14695840 | chr9:71737258 | TJP2.UTR5 | R | chr9:71736074-71737367 | Island |
| cg20592124 | chr2:65290738 | CEP68.UTR5 | R | | |
| cg01248385 | chr13:113636953 | MCF2L.GENEBODY | R | chr13:113635654-113635899 | S_Shore |
| cg08641990 | chr1:54822503 | SSBP3.GENEBODY | F | chr1:54821990-54822333 | S_Shore |
| cg14864994 | chr12:6496214 | LTBR.GENEBODY | F | chr12:6492889-6493522 | S_Shelf |
| cg20679581 | chr8:10222777 | MSRA.GENEBODY | F | | |
| cg24904739 | chr17:77959804 | TBC1D16.GENEBODY | R | | |
| cg25193885 | chr11:70328867 | SHANK2.GENEBODY | F | chr11:70332266-70333413 | N_Shelf |
| cg03127370 | chr16:2520192 | NTN3.TSS1500 | R | chr16:2521086-2525929 | N_Shore |
| cg21972567 | chr5:158350708 | EBF1.GENEBODY | F | | |
| cg25076197 | chr7:104681440 | MLL5.GENEBODY | F | | |
| cg15723222 | chr17:59222476 | BCAS3.GENEBODY | R | | |
| cg19420604 | chr17:60828406 | MARCH10.GENEBODY | F | | |
| cg03467001 | chr1:183857649 | RGL1.GENEBODY | R | | |
| cg04220881 | chr7:152524814 | ACTR3B.GENEBODY | F | | |
| cg13520715 | chr21:43135854 | C21orf129.GENEBODY | R | | |
| cg19272238 | chr17:76220898 | BIRC5.UTR3 | F | | |
| cg20628733 | chr1:3524459 | MEGF6.GENEBODY | R | chr1:3527619-3528078 | N_Shelf |
| cg26985681 | chr16:15135397 | NTAN1.GENEBODY | F | | |
| cg09853822 | chr17:4712456 | PLD2.GENEBODY | F | chr17:4710014-4710960 | S_Shore |
| cg15460454 | chr7:1974654 | MAD1L1.GENEBODY | R | chr7:1976258-1976464 | N_Shore |
| cg22532234 | chr19:40229215 | CLC.TSS1500 | R | | |
| cg04746649 | chr2:169401301 | LASS6.GENEBODY | R | | |
| cg09540626 | chr16:2590751 | PDPK1.GENEBODY | F | chr16:2588034-2588556 | S_Shelf |
| cg22244768 | chr17:55634551 | MSI2.GENEBODY | F | | |
| cg26504421 | chr17:71339361 | SDK2.GENEBODY | R | | |
| cg03091070 | chr6:169641288 | THBS2.GENEBODY | R | chr6:169641803-169642020 | N_Shore |
| cg19974120 | chr21:42839625 | TMPRSS2.GENEBODY | F | | |
| cg26344024 | chr3:71357195 | FOXP1.UTR5 | R | | |
| cg27126442 | chr4:106473806 | FLJ20184.UTR5 | F | | |
| cg01132579 | chr17:55588940 | MSI2.GENEBODY | R | | |
| cg03029090 | chr15:58812960 | LIPC.GENEBODY | R | | |
| cg06694239 | chr7:128807764 | TSPAN33.UTR3 | F | chr7:128809089-128809393 | N_Shore |
| cg08702745 | chr13:111138063 | COL4A2.GENEBODY | R | | |
| cg13793782 | chr9:135995093 | RALGDS.GENEBODY | F | chr9:135995969-135996954 | N_Shore |
| cg12248883 | chr11:6219573 | OR52W1.TSS1500 | F | | |
| cg00300637 | chr5:319433 | AHRR.GENEBODY | R | chr5:320788-323010 | N_Shore |
| cg01661001 | chr20:44439932 | UBE2C.TSS1500 | F | chr20:44440946-44441655 | N_Shore |
| cg04260891 | chr7:75514575 | RHBDD2.GENEBODY | F | | |
| cg12686273 | chr16:3085867 | CCDC64B.TSS1500 | F | chr16:3086283-3086968 | N_Shore |
| cg13363823 | chr1:40362907 | MYCL1.UTR3 | F | chr1:40366595-40368213 | N_Shelf |
| cg24688343 | chr17:61229244 | TANC2.GENEBODY | F | | |
| cg13844853 | chr1:94062769 | BCAR3.GENEBODY | F | | |
| cg20505457 | chr14:69388953 | ACTN1.GENEBODY | F | | |
| cg02923393 | chr16:1543003 | TELO2.TSS1500 | R | chr16:1543098-1544426 | N_Shore |
| cg10708739 | chr17:57465550 | YPEL2.GENEBODY | F | | |
| cg24791666 | chr3:39192215 | CSRNP1.UTR5 | F | chr3:39194241-39195359 | N_Shelf |
| cg00078299 | chr8:124284379 | ZHX1.UTR5 | R | chr8:124286209-124287666 | N_Shore |
| cg00474798 | chr2:84684489 | SUCLG1.GENEBODY | R | chr2:84686005-84686751 | N_Shore |
| cg01267165 | chr16:11681599 | LITAF.TSS1500 | F | chr16:11680008-11681275 | S_Shore |
| cg02405813 | chr6:83072522 | TPBG.TSS1500 | F | chr6:83073416-83075319 | N_Shore |
| cg08869273 | chr11:413594 | SIGIRR.UTR5 | R | chr11:416419-417650 | N_Shelf |
| cg09238199 | chr13:111097777 | COL4A2.GENEBODY | F | | |
| cg09333109 | chr19:51532580 | KLK11.TSS1500 | F | chr19:51535218-51535475 | N_Shelf |
| cg10813585 | chr1:114305956 | RSBN1.UTR3 | R | chr1:114301514-114302272 | S_Shelf |
| cg01262667 | chr19:19385393 | TM6SF2.TSS1500 | F | chr19:19383703-19384283 | S_Shore |
| cg03581459 | chr14:105780137 | PACS2.TSS1500 | F | chr14:105780339-105782027 | N_Shore |
| cg04961866 | chr16:3085686 | CCDC64B.TSS200 | R | chr16:3086283-3086968 | N_Shore |
| cg05263838 | chr2:227659406 | IRS1.UTR3 | F | chr2:227662082-227664912 | N_Shelf |
| cg11908131 | chr2:106685045 | C2orf40.GENEBODY | R | chr2:106681982-106682403 | S_Shelf |
| cg14377791 | chr19:40169912 | LOC400696.TSS200 | F | | |
| cg16081441 | chr17:74463560 | AANAT.TSS200 | R | | |
| cg20169296 | chr10:103367727 | RP11-529I10.4.GENEBODY | R | | |
| cg06915226 | chr2:70960959 | ADD2.UTR5 | F | | |
| cg07121199 | chr1:205537752 | MFSD4.TSS1500 | R | chr1:205537751-205538443 | Island |
| cg16929041 | chr17:26648951 | TMEM97.GENEBODY | F | chr17:26645290-26645614 | S_Shelf |
| cg01796143 | chr1:236687575 | LGALS8.UTR5 | R | chr1:236687071-236687608 | Island |
| cg01897496 | chr2:182545209 | NEUROD1.EXON1 | R | chr2:182547873-182549177 | N_Shelf |
| cg02445907 | chr6:36294404 | C6orf222.GENEBODY | F | | |
| cg10351284 | chr8:125617225 | MTSS1.GENEBODY | F | | |
| cg12778007 | chr14:72041267 | SIPA1L1.UTR5 | F | | |
| cg18319790 | chr14:100788777 | C14orf68.TSS1500 | R | | |
| cg21719937 | chr12:5564478 | NTF3.GENEBODY | R | | |
| cg21740204 | chr9:72873544 | SMC5.TSS1500 | F | chr9:72873689-72874264 | N_Shore |
| cg24657047 | chr15:77317370 | PSTPIP1.GENEBODY | R | chr15:77320150-77320456 | N_Shelf |
| cg26037504 | chr12:56113808 | RDH5.TSS1500 | F | chr12:56109798-56110298 | S_Shelf |
| cg26782539 | chr10:134061662 | STK32C.GENEBODY | F | | |
| cg02010776 | chr16:18938091 | SMG1.TSS1500 | R | chr16:18937030-18938126 | Island |
| cg03920522 | chr7:94047477 | COL1A2.GENEBODY | R | | |
| cg08946575 | chr1:85931072 | DDAH1.TSS200 | F | chr1:85929940-85931168 | Island |
| cg10039567 | chr1:112395111 | KCND3.GENEBODY | R | | |
| cg10316635 | chr1:62208645 | INADL.UTR5 | R | chr1:62207827-62208818 | Island |
| cg13973639 | chr6:42858710 | C6orf226.TSS200 | R | chr6:42858169-42858960 | Island |
| cg15653828 | chr6:34513187 | SPDEF.UTR5 | F | chr6:34512063-34512266 | S_Shore |
| cg19153095 | chr8:106811143 | ZFPM2.GENEBODY | F | | |
| cg02531277 | chr7:101558597 | CUX1.GENEBODY | R | chr7:101559402-101559625 | N_Shore |
| cg03036777 | chr1:33648819 | TRIM62.TSS1500 | F | chr1:33646613-33647878 | S_Shore |
| cg03621100 | chr12:104322919 | LOC253724.GENEBODY | F | chr12:104323017-104324811 | N_Shore |
| cg08706567 | chr1:43814983 | MPL.GENEBODY | F | chr1:43814305-43815277 | Island |
| cg08905114 | chr9:95897015 | NINJ1.TSS1500 | R | chr9:95896007-95897016 | Island |
| cg09702010 | chr19:51531393 | KLK11.TSS1500 | F | chr19:51535218-51535475 | N_Shelf |
| cg13264672 | chr7:39993440 | CDK13.GENEBODY | F | chr7:39988922-39991612 | S_Shore |
| cg14607161 | chr6:32084298 | ATF6B.GENEBODY | R | | |
| cg17141902 | chr9:95897216 | NINJ1.TSS1500 | F | chr9:95896007-95897016 | S_Shore |
| cg26681687 | chr4:77874155 | SEPT11.GENEBODY | F | chr4:77869440-77872122 | S_Shelf |
| cg05155047 | chr5:168719 | PLEKHG4B.GENEBODY | F | chr5:169578-169798 | N_Shore |
| cg10754794 | chr10:93916288 | CPEB3.GENEBODY | F | | |
| cg15744692 | chr15:45671195 | LOC145663.GENEBODY | F | chr15:45669970-45671029 | S_Shore |
| cg00955867 | chr1:160337038 | NHLH1.EXON1 | R | chr1:160340604-160340843 | N_Shelf |
| cg01470599 | chr9:38422794 | IGFBPL1.GENEBODY | F | chr9:38423947-38424584 | N_Shore |
| cg06508379 | chr19:19104064 | SFRS14.UTR3 | R | | |
| cg07491702 | chr15:43030817 | CDAN1.TSS1500 | R | chr15:43028413-43029346 | S_Shore |
| cg08673532 | chr16:87422913 | FBXO31.GENEBODY | F | chr16:87425137-87426677 | N_Shelf |
| cg16320329 | chr3:45981161 | FYCO1.GENEBODY | F | | |
| cg16321266 | chr7:129414894 | MIR183.TSS200 | F | chr7:129418286-129422509 | N_Shelf |
| cg17033622 | chr11:128568943 | FLI1.GENEBODY | R | chr11:128562671-128565011 | S_Shelf |
| cg18911861 | chr6:42858743 | C6orf226.TSS200 | R | chr6:42858169-42858960 | Island |
| cg22791543 | chr5:127416532 | FLJ33630.GENEBODY | R | chr5:127418594-127420512 | N_Shelf |
| cg27360567 | chr3:49941613 | MST1R.TSS1500 | F | chr3:49940099-49940431 | S_Shore |
| cg00764796 | chr16:433504 | LOC100134368.GENEBODY | R | chr16:431189-432810 | S_Shore |
| cg08843859 | chr1:184566345 | C1orf21.GENEBODY | F | | |
| cg09027133 | chr1:25945042 | MAN1C1.GENEBODY | F | chr1:25942661-25943191 | S_Shore |
| cg12145018 | chr14:103912507 | MARK3.GENEBODY | F | | |
| cg14087413 | chr9:35848330 | TMEM8B.GENEBODY | R | chr9:35846310-35846638 | S_Shore |
| cg18449361 | chr6:2689154 | MYLK4.GENEBODY | R | chr6:2685400-2685625 | S_Shelf |
| cg22393461 | chr1:10010658 | NMNAT1.UTR5 | F | | |
| cg22572454 | chr17:74463767 | AANAT.GENEBODY | F | | |
| cg00034154 | chr19:10337163 | S1PR2.UTR5 | F | chr19:10334618-10335511 | S_Shore |
| cg04656424 | chr16:88839844 | FAM38A.GENEBODY | F | chr16:88838112-88838459 | S_Shore |
| cg06561646 | chr17:35873369 | DUSP14.UTR3 | F | chr17:35872722-35873026 | S_Shore |
| cg07119973 | chr17:43109221 | DCAKD.GENEBODY | R | chr17:43111525-43111732 | N_Shelf |
| cg08637583 | chr15:44828465 | EIF3J.TSS1500 | R | chr15:44828637-44829949 | N_Shore |
| cg14453201 | chr5:320888 | AHRR.GENEBODY | R | chr5:320788-323010 | Island |
| cg15507486 | chr19:46627896 | IGFL3.EXON1 | F | | |
| cg21093368 | chr17:8055834 | PER1.TSS200 | F | chr17:8054550-8055835 | Island |
| cg26477169 | chr17:75196864 | SEC14L1.GENEBODY | R | | |
| cg26692003 | chr3:13063165 | IQSEC1.GENEBODY | R | | |
| cg27262398 | chr11:107578793 | SLN.UTR5 | R | | |
| cg27559097 | chr2:144987743 | GTDC1.UTR5 | F | | |
| cg18407095 | chr2:26846581 | CIB4.GENEBODY | R | | |
| cg00225157 | chr6:33173038 | HSD17B8.GENEBODY | F | chr6:33172289-33172823 | S_Shore |
| cg00648184 | chr17:60828157 | MARCH10.GENEBODY | R | | |
| cg03035849 | chr6:91003200 | BACH2.UTR5 | R | chr6:91004795-91006944 | N_Shore |
| cg05294243 | chr19:51569106 | KLK13.TSS1500 | F | chr19:51568154-51568412 | S_Shore |
| cg05581469 | chr12:49413435 | MLL2.UTR3 | F | chr12:49412299-49412756 | S_Shore |
| cg13296983 | chr12:6436553 | PLEKHG6.GENEBODY | F | chr12:6438272-6438931 | N_Shore |
| cg01448470 | chr1:18518226 | IGSF21.GENEBODY | R | | |
| cg06747907 | chr1:16091100 | FBLIM1.UTR5 | R | | |
| cg04138378 | chr7:95849192 | MIR591.TSS200 | R | | |
| cg15152301 | chr3:50425602 | CACNA2D2.GENEBODY | F | | |
| cg25882455 | chr22:38599073 | MAFF.EXON1 | F | chr22:38597785-38598988 | S_Shore |
| cg02862897 | chr17:79793088 | DYSFIP1.TSS200 | F | chr17:79790530-79791553 | S_Shore |
| cg05036991 | chr2:155089854 | GALNT13.GENEBODY | F | | |
| cg13508369 | chr9:129884026 | ANGPTL2.UTR5 | R | | |
| cg15076368 | chr3:39224943 | XIRP1.UTR3 | R | chr3:39222280-39222670 | S_Shelf |
| cg15204874 | chr1:54822298 | SSBP3.GENEBODY | R | chr1:54821990-54822333 | Island |
| cg18211633 | chr12:122205609 | TMEM120B.GENEBODY | R | | |
| cg19287817 | chr19:15391339 | BRD4.TSS200 | F | | |
| cg00985958 | chr6:71605996 | B3GAT2.GENEBODY | F | | |
| cg04617549 | chr19:51569622 | KLK13.TSS1500 | F | chr19:51568154-51568412 | S_Shore |
| cg04918002 | chr16:87423124 | FBXO31.GENEBODY | R | chr16:87425137-87426677 | N_Shelf |
| cg05487134 | chr17:40489569 | STAT3.GENEBODY | F | | |
| cg05928980 | chr1:29567425 | PTPRU.GENEBODY | R | chr1:29562745-29565307 | S_Shelf |
| cg07824564 | chr1:179050911 | TOR3A.TSS200 | F | chr1:179051075-179051948 | N_Shore |
| cg13261623 | chr17:74462685 | AANAT.GENEBODY | R | | |
| cg13698883 | chr9:38422614 | IGFBPL1.GENEBODY | F | chr9:38423947-38424584 | N_Shore |
| cg26279814 | chr17:37617607 | CDK12.TSS200 | F | chr17:37617531-37618045 | Island |
| cg00193613 | chr17:77042810 | C1QTNF1.GENEBODY | R | chr17:77043741-77044489 | N_Shore |
| cg06201861 | chr16:84877964 | CRISPLD2.GENEBODY | F | | |
| cg23551605 | chr2:236442942 | AGAP1.GENEBODY | F | chr2:236443815-236444025 | N_Shore |
| cg25482454 | chr2:85988369 | ATOH8.GENEBODY | R | | |
| cg06091094 | chr11:44927766 | TSPAN18.UTR5 | F | | |
| cg10426951 | chr1:54821910 | SSBP3.GENEBODY | F | chr1:54821990-54822333 | N_Shore |
| cg17490445 | chr7:32623673 | AVL9.UTR3 | F | | |
| cg17699214 | chr4:40890013 | APBB2.GENEBODY | R | | |
| cg00564723 | chr10:75632066 | CAMK2G.GENEBODY | F | chr10:75633600-75634796 | N_Shore |
| cg04999036 | chr15:42566375 | TMEM87A.TSS1500 | R | chr15:42566198-42566404 | Island |
| cg06130787 | chr19:51523550 | KLK10.TSS1500 | F | chr19:51522004-51522803 | S_Shore |
| cg09923953 | chr19:51525638 | KLK11.UTR3 | F | chr19:51522004-51522803 | S_Shelf |
| cg14414406 | chr9:100326029 | TMOD1.GENEBODY | F | | |
| cg19392379 | chr10:123909426 | TACC2.GENEBODY | R | | |
| cg10585321 | chr9:135995491 | RALGDS.GENEBODY | R | chr9:135995969-135996954 | N_Shore |
| cg12581298 | chr7:1080836 | C7orf50.GENEBODY | R | chr7:1080835-1081069 | Island |
| cg13548543 | chr9:34460044 | DNAI1.GENEBODY | F | chr9:34457375-34458892 | S_Shore |
| cg18322589 | chr10:123909456 | TACC2.GENEBODY | F | | |
| cg25285041 | chr1:41627631 | SCMH1.UTR5 | R | | |
| cg00664743 | chr22:31300253 | OSBP2.GENEBODY | R | chr22:31301702-31302133 | N_Shore |
| cg03717830 | chr6:30182244 | TRIM26.TSS1500 | R | chr6:30180933-30182187 | S_Shore |
| cg18263988 | chr3:111582491 | PHLDB2.UTR5 | R | chr3:111578237-111578739 | S_Shelf |
| cg02312706 | chr16:87384599 | FBXO31.GENEBODY | F | | |
| cg03762081 | chr19:51523565 | KLK10.TSS1500 | F | chr19:51522004-51522803 | S_Shore |
| cg17585356 | chr17:78857121 | RPTOR.GENEBODY | R | chr17:78857630-78857866 | N_Shore |
| cg19727124 | chr1:6553227 | PLEKHG5.GENEBODY | R | chr1:6550083-6551115 | S_Shelf |
| cg23045716 | chr4:1201881 | LOC100130872.GENEBODY | R | chr4:1202956-1203200 | N_Shore |
| cg26530485 | chr1:23496304 | LUZP1.TSS1500 | F | | |
| cg27119919 | chr11:60684332 | TMEM109.UTR5 | F | chr11:60681552-60682268 | S_Shelf |
| cg11634198 | chr17:37764158 | NEUROD2.UTR5 | F | chr17:37764092-37764304 | Island |
| cg15798221 | chr17:62773704 | LOC146880.GENEBODY | R | chr17:62774366-62777797 | N_Shore |
| cg24112733 | chr7:38255578 | STARD3NL.GENEBODY | F | | |
| cg08897952 | chr11:126229062 | ST3GAL4.UTR5 | R | chr11:126225355-126226073 | S_Shelf |
| cg13969327 | chr7:127526253 | SND1.GENEBODY | F | | |
| cg18253971 | chr18:21900171 | OSBPL1A.GENEBODY | F | | |
| cg20104456 | chr19:51568523 | KLK13.TSS200 | R | chr19:51568154-51568412 | S_Shore |
| cg24247667 | chr17:74463439 | AANAT.TSS200 | F | | |
| cg24901995 | chr11:888570 | CHID1.GENEBODY | R | chr11:888684-888981 | N_Shore |
| cg14225450 | chr6:169637832 | THBS2.GENEBODY | F | chr6:169641803-169642020 | N_Shelf |
| cg01119831 | chr7:2119608 | MAD1L1.GENEBODY | R | chr7:2116367-2116781 | S_Shelf |
| cg01593787 | chr13:111182949 | RAB20.GENEBODY | F | | |
| cg16451995 | chr16:8942340 | PMM2.UTR3 | R | | |
| cg17523945 | chr19:46732351 | IGFL1.TSS1500 | F | | |
| cg21323630 | chr3:51995784 | PCBP4.UTR5 | R | | |
| cg23712594 | chr1:226591890 | PARP1.GENEBODY | R | chr1:226595333-226595939 | N_Shelf |
| cg17170262 | chr1:184715203 | EDEM3.GENEBODY | F | | |
| cg05338397 | chr1:15466556 | C1orf126.GENEBODY | R | | |
| cg23682934 | chr17:42885322 | GJC1.UTR5 | R | | |
| cg06195280 | chr17:6658706 | XAF1.TSS1500 | F | | |
| cg01766396 | chr2:198436483 | RFTN2.UTR3 | F | | |
| cg07990556 | chr4:159792176 | FNIP2.GENEBODY | F | | |
| cg24899626 | chr8:10512724 | RP1L1.TSS200 | F | | |
| cg19028997 | chr1:205603040 | ELK4.TSS1500 | R | chr1:205600493-205601432 | S_Shore |
| cg12562403 | chr12:3214145 | TSPAN9.UTR5 | R | | |
| cg08228249 | chr22:22062300 | YPEL1.GENEBODY | F | chr22:22058079-22058304 | S_Shelf |
| cg27634115 | chr2:42423477 | EML4.GENEBODY | F | | |
| cg03861347 | chr20:46412713 | SULF2.UTR5 | F | chr20:46413742-46415507 | N_Shore |
| cg07941953 | chr20:10508668 | C20orf94.GENEBODY | R | | |
| cg03187411 | chr7:110482491 | IMMP2L.GENEBODY | R | | |
| cg03223734 | chr20:20180211 | C20orf26.GENEBODY | R | | |
| cg23793194 | chr17:37763735 | NEUROD2.UTR5 | R | chr17:37764092-37764304 | N_Shore |
| cg07831164 | chr16:10836225 | NUBP1.TSS1500 | R | chr16:10837584-10838196 | N_Shore |
| cg20674206 | chr1:63004738 | DOCK7.GENEBODY | F | | |
| cg22721827 | chr16:2520958 | NTN3.TSS1500 | F | chr16:2521086-2525929 | N_Shore |
| cg27096416 | chr7:140599947 | BRAF.GENEBODY | F | | |
| cg03419151 | chr17:6658728 | XAF1.TSS1500 | F | | |
| cg05420057 | chr17:36721297 | SRCIN1.GENEBODY | F | chr17:36719544-36719938 | S_Shore |
| cg05950943 | chr16:2520971 | NTN3.TSS1500 | F | chr16:2521086-2525929 | N_Shore |
| cg03243893 | chr8:2005683 | MYOM2.GENEBODY | R | chr8:2003809-2004059 | S_Shore |
| cg20255723 | chr22:45073031 | PRR5.UTR5 | R | | |
| cg25275372 | chr17:964596 | ABR.GENEBODY | F | | |
| cg00284708 | chr8:43147581 | POTEA.TSS200 | F | | |
| cg09305096 | chr2:189191655 | GULP1.UTR5 | R | | |
| cg16696035 | chr17:17126176 | FLCN.GENEBODY | R | chr17:17124698-17124931 | S_Shore |
| cg02966899 | chr15:64793532 | ZNF609.GENEBODY | R | | |
| cg09606564 | chr17:19290353 | MFAP4.GENEBODY | F | | |
| cg00332588 | chr8:43147564 | POTEA.TSS200 | R | | |
| cg08041350 | chr17:25819112 | KSR1.UTR5 | R | | |
| cg14074652 | chr9:136662289 | VAV2.GENEBODY | F | chr9:136659111-136659329 | S_Shelf |
| cg19963589 | chr1:54822103 | SSBP3.GENEBODY | F | chr1:54821990-54822333 | Island |
| cg09309024 | chr16:29837267 | MVP.UTR5 | F | | |
| cg11952344 | chr17:80398854 | HEXDC.GENEBODY | F | chr17:80397431-80397642 | S_Shore |
| cg20431135 | chr17:19290762 | MFAP4.TSS1500 | F | | |
| cg19155699 | chr3:196050902 | TM4SF19.GENEBODY | F | | |
| cg23704362 | chr8:67405742 | C8orf46.EXON1 | F | | |
| cg19991383 | chr3:55034387 | CACNA2D3.GENEBODY | F | | |
| cg00172603 | chr1:54822031 | SSBP3.GENEBODY | F | chr1:54821990-54822333 | Island |
| cg06431681 | chr2:202330990 | STRADB.GENEBODY | F | | |
| cg14461974 | chr2:3318039 | TSSC1.GENEBODY | R | chr2:3317676-3317935 | S_Shore |
| cg15119221 | chr17:19290755 | MFAP4.TSS1500 | F | | |
| cg21831649 | chr1:16050377 | PLEKHM2.GENEBODY | R | chr1:16053616-16053975 | N_Shelf |
| cg06087554 | chr16:31151917 | PRSS36.GENEBODY | R | chr16:31153556-31154474 | N_Shore |
| cg01293647 | chr17:37764264 | NEUROD2.TSS200 | R | chr17:37764092-37764304 | Island |
| cg14281144 | chr9:38421899 | IGFBPL1.GENEBODY | F | chr9:38423947-38424584 | N_Shelf |
| cg13726218 | chr13:72438249 | DACH1.GENEBODY | R | chr13:72439362-72440059 | N_Shore |
| cg17892159 | chr2:42274610 | PKDCC.TSS1500 | F | | |
| cg10414350 | chr8:10210282 | MSRA.GENEBODY | R | | |
| cg20326410 | chr1:53600821 | SLC1A7.GENEBODY | R | | |
| cg06785060 | chr1:42180981 | HIVEP3.UTR5 | F | | |
| cg07685557 | chr16:85685994 | KIAA0182.GENEBODY | F | chr16:85684107-85684491 | S_Shore |
| cg08520443 | chr17:746641 | NXN.GENEBODY | R | chr17:744617-744907 | S_Shore |
| cg17322655 | chr2:242802127 | PDCD1.TSS1500 | F | chr2:242805752-242806034 | N_Shelf |
| cg00545232 | chr13:93923232 | GPC6.GENEBODY | R | | |
| cg11019127 | chr11:115369647 | CADM1.GENEBODY | F | chr11:115373574-115375881 | N_Shelf |
| cg08151442 | chr2:238632428 | LRRFIP1.GENEBODY | F | | |
| cg16719771 | chr8:43147554 | POTEA.TSS200 | R | | |
| cg20944379 | chr1:207069392 | IL24.TSS1500 | R | | |
| cg03679521 | chr17:37764153 | NEUROD2.UTR5 | F | chr17:37764092-37764304 | Island |
| cg09440603 | chr21:43105616 | NCRNA00111.GENEBODY | F | chr21:43109349-43109687 | N_Shelf |
| cg07360805 | chr20:36148457 | BLCAP.UTR5 | F | chr20:36148603-36150136 | N_Shore |
| cg01796800 | chr17:55595732 | MSI2.GENEBODY | F | | |
| cg06429887 | chr14:24600500 | FITM1.TSS200 | F | chr14:24601498-24601700 | N_Shore |
| cg10603757 | chr22:37621745 | RAC2.UTR3 | R | | |
| cg13440795 | chr8:10210267 | MSRA.GENEBODY | R | | |
| cg03889044 | chr2:242802099 | PDCD1.TSS1500 | F | chr2:242805752-242806034 | N_Shelf |
| cg08200851 | chr6:5472095 | FARS2.GENEBODY | F | | |
| cg09414280 | chr12:120130253 | CIT.GENEBODY | R | chr12:120127973-120128291 | S_Shore |
| cg21641186 | chr3:126200897 | UROC1.UTR3 | R | | |
| cg21830368 | chr17:75373327 | SEPT9.UTR5 | R | chr17:75368688-75370506 | S_Shelf |
| cg04951371 | chr2:3317860 | TSSC1.GENEBODY | F | chr2:3317676-3317935 | Island |
| cg21557724 | chr2:3317928 | TSSC1.GENEBODY | R | chr2:3317676-3317935 | Island |
| cg12716832 | chr22:45073165 | PRR5.UTR5 | R | | |
| cg04436083 | chr4:24567220 | DHX15.GENEBODY | R | | |
| cg11125249 | chr3:148737622 | GYG1.GENEBODY | F | | |
| cg11993925 | chr19:44307056 | LYPD5.TSS1500 | F | chr19:44302665-44303176 | S_Shelf |
| cg15293827 | chr4:2827194 | SH3BP2.GENEBODY | F | | |
| cg27531366 | chr1:27627058 | WDTC1.GENEBODY | F | | |
| cg01943029 | chr16:31715088 | C16orf67.GENEBODY | F | chr16:31711839-31712343 | S_Shelf |
| cg13027937 | chr7:152525220 | ACTR3B.GENEBODY | F | | |
| cg16623710 | chr22:45073049 | PRR5.UTR5 | F | | |
| cg00679520 | chr12:63133578 | PPM1H.GENEBODY | F | | |
| cg03742724 | chr6:161838997 | PARK2.GENEBODY | R | | |
| cg14300048 | chr9:72046240 | APBA1.UTR3 | F | | |
| cg09720012 | chr4:108637177 | PAPSS1.GENEBODY | R | chr4:108640845-108641835 | N_Shelf |
| cg22689630 | chr19:51568918 | KLK13.TSS1500 | F | chr19:51568154-51568412 | S_Shore |
| cg27068996 | chr19:51513883 | KLK9.TSS1500 | F | | |
| cg09535960 | chr8:23193704 | LOXL2.GENEBODY | F | | |
| cg02734358 | chr4:90227074 | GPRIN3.UTR5 | R | chr4:90228714-90229010 | N_Shore |
| cg03118626 | chr19:51523252 | KLK10.UTR5 | R | chr19:51522004-51522803 | S_Shore |
| cg06358171 | chr1:54822008 | SSBP3.GENEBODY | R | chr1:54821990-54822333 | Island |
| cg06440275 | chr8:2005701 | MYOM2.GENEBODY | R | chr8:2003809-2004059 | S_Shore |
| cg09393675 | chr17:37764250 | NEUROD2.TSS200 | R | chr17:37764092-37764304 | Island |
| cg20164964 | chr12:3213850 | TSPAN9.UTR5 | R | | |
| cg02175739 | chr11:875980 | CHID1.GENEBODY | R | | |
| cg08969377 | chr17:80915263 | B3GNTL1.GENEBODY | R | chr17:80918218-80919258 | N_Shelf |
| cg00317626 | chr1:53600741 | SLC1A7.GENEBODY | F | | |
| cg09382492 | chr17:74463681 | AANAT.GENEBODY | F | | |
| cg02516456 | chr11:1025091 | MUC6.GENEBODY | R | chr11:1024746-1025359 | Island |
| cg03737367 | chr19:50005045 | MIR150.TSS1500 | R | | |
| cg13446863 | chr9:139259726 | CARD9.GENEBODY | R | chr9:139257842-139259075 | S_Shore |
| cg02048220 | chr14:91141833 | TTC7B.GENEBODY | R | | |
| cg06760830 | chr1:47079672 | MOBKL2C.GENEBODY | F | chr1:47078580-47078967 | S_Shore |
| cg11912239 | chr17:76220608 | BIRC5.UTR3 | F | | |
| cg13525276 | chr14:81426012 | TSHR.GENEBODY | F | | |
| cg14025021 | chr15:43809510 | MAP1A.TSS1500 | F | | |
| cg16960943 | chr2:236417185 | AGAP1.GENEBODY | R | | |
| cg17741629 | chr13:50024703 | SETDB2.TSS1500 | R | | |
| cg04135110 | chr5:346695 | AHRR.GENEBODY | R | chr5:343449-344535 | S_Shelf |
| cg18722086 | chr3:113380072 | KIAA2018.GENEBODY | R | | |
| cg23141629 | chr13:45792582 | GTF2F2.GENEBODY | R | | |
| cg23571857 | chr17:6658898 | XAF1.TSS1500 | F | | |
| cg05416911 | chr12:7176049 | C1S.GENEBODY | F | | |
| cg15341350 | chr12:119446891 | SRRM4.GENEBODY | F | | |
| cg15382095 | chr17:37762367 | NEUROD2.GENEBODY | F | chr17:37761691-37763551 | Island |
| cg00795812 | chr2:242802009 | PDCD1.TSS1500 | F | chr2:242805752-242806034 | N_Shelf |
| cg14931683 | chr17:35872880 | DUSP14.GENEBODY | R | chr17:35872722-35873026 | Island |
| cg12097330 | chr17:79050221 | BAIAP2.GENEBODY | R | chr17:79051892-79052534 | N_Shore |
| cg12517843 | chr19:17361126 | USHBP1.GENEBODY | F | chr19:17356979-17357500 | S_Shelf |
| cg25216196 | chr17:19290520 | MFAP4.TSS200 | R | | |
| cg27051683 | chr2:242802069 | PDCD1.TSS1500 | F | chr2:242805752-242806034 | N_Shelf |
| cg07290310 | chr3:125741734 | SLC41A3.GENEBODY | R | | |
| cg16360668 | chr1:19265059 | IFFO2.GENEBODY | R | | |
| cg12237784 | chr7:44187017 | GCK.GENEBODY | F | chr7:44184770-44185695 | S_Shore |
| cg00175150 | chr1:150486261 | ECM1.UTR3 | R | chr1:150488036-150488416 | N_Shore |
| cg14292870 | chr12:6887543 | LAG3.GENEBODY | F | | |
| cg16007395 | chr1:33231070 | KIAA1522.GENEBODY | F | | |
| cg24980413 | chr5:346987 | AHRR.GENEBODY | R | chr5:343449-344535 | S_Shelf |
| cg10827768 | chr2:28387339 | BRE.GENEBODY | R | | |
| cg20340501 | chr2:233141525 | DIS3L2.GENEBODY | F | | |
| cg23271234 | chr2:71126275 | VAX2.TSS1500 | F | chr2:71126720-71129209 | N_Shore |
| cg03162164 | chr7:790384 | HEATR2.GENEBODY | F | chr7:791984-792238 | N_Shore |
| cg07727884 | chr17:19290503 | MFAP4.TSS200 | R | | |
| cg27417749 | chr15:69449518 | MIR548H4.GENEBODY | R | chr15:69452451-69453939 | N_Shelf |
| cg06603325 | chr17:39887725 | HAP1.GENEBODY | F | chr17:39889956-39891489 | N_Shelf |
| cg03446876 | chr7:129414697 | MIR96.TSS200 | R | chr7:129418286-129422509 | N_Shelf |
| cg03454775 | chr8:2012595 | MYOM2.GENEBODY | R | | |
| cg09427374 | chr2:86095320 | ST3GAL5.TSS1500 | R | | |
| cg10825315 | chr14:81425912 | TSHR.GENEBODY | R | | |
| cg17096191 | chr1:162039224 | NOS1AP.TSS1500 | F | chr1:162039450-162040052 | N_Shore |
| cg19784401 | chr11:46457992 | AMBRA1.GENEBODY | F | | |
| cg04876127 | chr1:33618450 | TRIM62.GENEBODY | R | | |
| cg11312087 | chr13:100944053 | PCCA.GENEBODY | R | | |
| cg14682005 | chr6:15337603 | JARID2.GENEBODY | R | | |
| cg16198315 | chr13:72438678 | DACH1.GENEBODY | R | chr13:72439362-72440059 | N_Shore |
| cg20721467 | chr10:76597927 | MYST4.UTR5 | F | | |
| cg23894058 | chr19:51537828 | KLK12.GENEBODY | F | chr19:51535218-51535475 | S_Shelf |
| cg00892353 | chr8:124247948 | C8orf76.GENEBODY | R | | |
| cg08472243 | chr16:427912 | TMEM8A.GENEBODY | F | chr16:426038-426290 | S_Shore |
| cg10135894 | chr4:7862655 | AFAP1.GENEBODY | F | | |
| cg01146614 | chr6:159235211 | EZR.GENEBODY | F | chr6:159239081-159240775 | N_Shelf |
| cg03470159 | chr8:10261798 | MSRA.GENEBODY | R | chr8:10261900-10262222 | N_Shore |
| cg04629204 | chr1:26348325 | EXTL1.EXON1 | F | | |
| cg15511928 | chr1:201895256 | LMOD1.GENEBODY | R | | |
| cg18482164 | chr17:37764118 | NEUROD2.UTR5 | R | chr17:37764092-37764304 | Island |
| cg23732608 | chr20:20180469 | C20orf26.GENEBODY | F | | |
| cg24355809 | chr17:746574 | NXN.GENEBODY | R | chr17:744617-744907 | S_Shore |
| cg19595760 | chr1:25948511 | MAN1C1.GENEBODY | F | chr1:25944099-25944772 | S_Shelf |
| cg08853318 | chr17:71089186 | SLC39A11.TSS1500 | F | | |
| cg21706038 | chr2:3317693 | TSSC1.GENEBODY | R | chr2:3317676-3317935 | Island |
| cg26577529 | chr12:52866845 | KRT6C.GENEBODY | R | | |
| cg01179256 | chr12:105141965 | CHST11.GENEBODY | F | | |
| cg02069944 | chr17:79191523 | AZI1.GENEBODY | R | | |
| cg13409077 | chr9:35618528 | CD72.TSS200 | R | chr9:35616980-35617324 | S_Shore |
| cg13700197 | chr9:98221975 | PTCH1.GENEBODY | R | | |
| cg13867915 | chr1:27050039 | ARID1A.GENEBODY | F | | |
| cg24083274 | chr7:121977191 | CADPS2.GENEBODY | F | | |
| cg05337753 | chr17:75373407 | SEPT9.UTR5 | F | chr17:75368688-75370506 | S_Shelf |
| cg07533819 | chr5:71407465 | MAP1B.GENEBODY | F | chr5:71402877-71404868 | S_Shelf |
| cg17327404 | chr1:33231109 | KIAA1522.GENEBODY | R | | |
| cg02627403 | chr17:73823769 | UNC13D.UTR3 | R | | |
| cg11552585 | chr19:3196206 | NCLN.GENEBODY | F | chr19:3196188-3196393 | Island |
| cg13406860 | chr9:132881336 | GPR107.GENEBODY | F | | |
| cg17492795 | chr1:214637903 | PTPN14.GENEBODY | R | | |
| cg18590709 | chr17:49122713 | SPAG9.GENEBODY | R | | |
| cg24054871 | chr22:45073148 | PRR5.UTR5 | R | | |
| cg13404038 | chr9:126123344 | CRB2.GENEBODY | F | | |
| cg16797751 | chr7:105290218 | ATXN7L1.GENEBODY | F | | |
| cg04607412 | chr22:45073157 | PRR5.UTR5 | R | | |
| cg11213150 | chr9:129884457 | ANGPTL2.UTR5 | F | | |
| cg19921389 | chr3:128780164 | GP9.UTR5 | R | | |
| cg10784542 | chr6:169625369 | THBS2.GENEBODY | R | chr6:169622297-169622520 | S_Shelf |
| cg21303745 | chr17:77959826 | TBC1D16.GENEBODY | F | | |
| cg06786238 | chr1:33231190 | KIAA1522.GENEBODY | R | | |
| cg09021393 | chr7:95849568 | MIR591.TSS1500 | R | | |
| cg14121282 | chr9:137268074 | RXRA.GENEBODY | F | | |
| cg17697739 | chr6:1873340 | GMDS.GENEBODY | R | | |
| cg21885317 | chr8:124248094 | C8orf76.GENEBODY | F | | |
| cg01429256 | chr17:17687834 | RAI1.UTR5 | F | chr17:17685016-17687240 | S_Shore |
| cg02582754 | chr6:160327345 | MAS1.TSS1500 | R | | |
| cg07997493 | chr17:37761947 | NEUROD2.GENEBODY | R | chr17:37761691-37763551 | Island |
| cg21565781 | chr12:123892621 | SETD8.UTR3 | R | | |
| cg02381521 | chr21:42775934 | MX2.GENEBODY | F | | |
| cg09768257 | chr8:11628346 | NEIL2.UTR5 | R | chr8:11627091-11627668 | S_Shore |
| cg12205591 | chr21:44588552 | CRYAA.TSS1500 | F | chr21:44590612-44590826 | N_Shelf |
| cg18875674 | chr11:73026651 | ARHGEF17.GENEBODY | F | | |
| cg08958913 | chr19:40193890 | LGALS14.TSS1500 | R | | |
| cg20712415 | chr3:159570903 | SCHIP1.GENEBODY | F | | |
| cg24653772 | chr7:788989 | HEATR2.GENEBODY | F | chr7:791984-792238 | N_Shelf |
| cg10529401 | chr1:53587046 | SLC1A7.GENEBODY | F | | |
| cg13909661 | chr12:26386833 | SSPN.UTR3 | R | | |
| cg14388093 | chr9:133342842 | ASS1.GENEBODY | F | | |
| cg27554156 | chr12:13248725 | GSG1.GENEBODY | F | | |
| cg02807008 | chr19:1910258 | ADAT3.UTR5 | R | chr19:1911923-1913406 | N_Shore |
| cg14448919 | chr5:435040 | AHRR.UTR3 | F | | |
| cg17873750 | chr1:116382984 | NHLH2.UTR5 | F | chr1:116380359-116382364 | S_Shore |
| cg26844077 | chr16:85658855 | KIAA0182.UTR5 | F | chr16:85658742-85658998 | Island |
| cg26902557 | chr11:1585852 | DUSP8.GENEBODY | F | | |
| cg06479434 | chr7:128395929 | CALU.GENEBODY | R | | |
| cg08811581 | chr11:16902852 | PLEKHA7.GENEBODY | F | | |
| cg08902205 | chr16:85658997 | KIAA0182.UTR5 | F | chr16:85658742-85658998 | Island |
| cg18023842 | chr1:160336475 | NHLH1.TSS1500 | F | | |
| cg21448423 | chr1:55013956 | ACOT11.EXON1 | R | | |
| cg07923746 | chr1:179050803 | TOR3A.TSS1500 | F | chr1:179051075-179051948 | N_Shore |
| cg10987071 | chr2:217276744 | SMARCAL1.TSS1500 | F | chr2:217277037-217277557 | N_Shore |
| cg07397891 | chr1:246582071 | SMYD3.GENEBODY | F | | |
| cg09997760 | chr3:179169556 | GNB4.TSS200 | R | chr3:179168735-179169593 | Island |
| cg18405786 | chr19:18746985 | KLHL26.TSS1500 | F | | |
| cg24852561 | chr13:100340080 | CLYBL.GENEBODY | F | | |
| cg04702527 | chr4:139940407 | CCRN4L.GENEBODY | F | chr4:139936443-139937428 | S_Shelf |
| cg23994112 | chr17:75373457 | SEPT9.UTR5 | R | chr17:75368688-75370506 | S_Shelf |
| cg05428433 | chr16:3614067 | NLRC3.GENEBODY | F | chr16:3614316-3614601 | N_Shore |
| cg09138688 | chr5:49963801 | PARP8.GENEBODY | F | chr5:49962267-49963777 | S_Shore |
| cg11367404 | chr4:170180146 | SH3RF1.GENEBODY | R | | |
| cg15179499 | chr5:362529 | AHRR.GENEBODY | R | chr5:356570-359526 | S_Shelf |
| cg20782117 | chr7:95865631 | SLC25A13.GENEBODY | R | | |
| cg23296791 | chr19:40229766 | CLC.TSS1500 | F | | |
| cg24524634 | chr7:2800976 | GNA12.GENEBODY | F | chr7:2802440-2802698 | N_Shore |
| cg09309711 | chr19:17322661 | MYO9B.UTR3 | F | chr19:17322471-17322988 | Island |
| cg18254848 | chr19:40227949 | CLC.GENEBODY | F | | |
| cg08157318 | chr12:102224140 | GNPTAB.GENEBODY | R | chr12:102224262-102224614 | N_Shore |
| cg10833439 | chr21:42793818 | MX1.UTR5 | F | chr21:42792293-42792704 | S_Shore |
| cg12102573 | chr17:27924969 | ANKRD13B.GENEBODY | F | chr17:27919874-27921105 | S_Shelf |
| cg00756860 | chr1:144852047 | PDE4DIP.UTR3 | R | | |
| cg01120953 | chr5:149643466 | CAMK2A.GENEBODY | R | | |
| cg09980085 | chr19:40174352 | LOC400696.UTR3 | F | | |
| cg13913247 | chr4:141207827 | SCOC.UTR5 | F | | |
| cg17568996 | chr22:42828125 | NFAM1.GENEBODY | F | | |
| cg04571652 | chr3:192959762 | MGC2889.GENEBODY | F | chr3:192958709-192959373 | S_Shore |
| cg06256596 | chr16:66511944 | BEAN.GENEBODY | R | | |
| cg06772157 | chr7:100728711 | TRIM56.TSS200 | F | chr7:100732088-100732424 | N_Shelf |
| cg23202177 | chr1:116383238 | NHLH2.UTR5 | R | chr1:116380359-116382364 | S_Shore |
| cg16419345 | chr17:73976089 | ACOX1.TSS1500 | R | chr17:73974971-73975731 | S_Shore |
| cg22807608 | chr17:17594719 | RAI1.UTR5 | F | chr17:17597824-17598398 | N_Shelf |
| cg26772172 | chr17:77474395 | HRNBP3.UTR5 | R | | |
| cg02566157 | chr11:61025815 | VWCE.UTR3 | R | | |
| cg02851203 | chr1:54808689 | SSBP3.GENEBODY | F | | |
| cg08247612 | chr1:160336486 | NHLH1.TSS1500 | F | | |
| cg20367923 | chr6:169624501 | THBS2.GENEBODY | R | chr6:169622297-169622520 | S_Shore |
| cg22460466 | chr7:1896154 | MAD1L1.GENEBODY | F | chr7:1896090-1896451 | Island |
| cg04530460 | chr6:42858550 | C6orf226.UTR5 | F | chr6:42858169-42858960 | Island |
| cg07424295 | chr10:92980030 | PCGF5.TSS1500 | R | chr10:92980347-92980832 | N_Shore |
| cg09135866 | chr19:53238498 | ZNF611.TSS200 | F | chr19:53237861-53238499 | Island |
| cg22102078 | chr6:130182512 | C6orf191.TSS200 | R | | |
| cg04155280 | chr17:26692719 | SEBOX.GENEBODY | F | | |
| cg02997295 | chr6:169642301 | THBS2.GENEBODY | F | chr6:169641803-169642020 | S_Shore |
| cg08399444 | chr12:13248548 | GSG1.GENEBODY | F | | |
| cg27379887 | chr20:1448498 | NSFL1C.TSS200 | R | chr20:1446936-1447729 | S_Shore |
| cg21831485 | chr7:31594014 | CCDC129.GENEBODY | F | | |
| cg20292636 | chr1:204328377 | PLEKHA6.UTR5 | F | | |
| cg23316807 | chr19:56628171 | ZNF787.UTR5 | F | chr19:56631771-56633076 | N_Shelf |
| cg03361664 | chr11:3187760 | OSBPL5.TSS1500 | R | chr11:3186131-3186768 | S_Shore |
| cg11759917 | chr17:79655302 | HGS.GENEBODY | R | chr17:79655162-79655515 | Island |
| cg17861836 | chr17:79202505 | C17orf56.UTR3 | F | | |
| cg19046189 | chr19:49316732 | HSD17B14.GENEBODY | F | chr19:49315545-49315765 | S_Shore |
| cg25342005 | chr1:33648656 | TRIM62.TSS1500 | R | chr1:33646613-33647878 | S_Shore |
| cg08301181 | chr17:73780373 | UNK.TSS1500 | R | chr17:73780846-73781308 | N_Shore |
| cg23490611 | chrX:134475099 | ZNF75D.UTR5 | R | chrX:134477583-134477999 | N_Shelf |
| cg01157146 | chr21:42877638 | TMPRSS2.GENEBODY | R | chr21:42878751-42880674 | N_Shore |
| cg03155027 | chr3:49907692 | CAMKV.TSS1500 | F | chr3:49906737-49907482 | S_Shore |
| cg14269510 | chr13:47204488 | LRCH1.GENEBODY | R | | |
| cg18849256 | chr12:32309670 | BICD1.GENEBODY | F | | |
| cg21930140 | chr21:42796537 | MX1.TSS1500 | F | chr21:42798146-42798884 | N_Shore |
| cg22377043 | chr15:43809486 | MAP1A.TSS1500 | F | | |
| cg00928231 | chr11:67195771 | RPS6KB2.TSS200 | F | chr11:67195800-67196190 | N_Shore |
| cg04329478 | chr19:40169839 | LOC400696.TSS200 | F | | |
| cg05070839 | chr8:10261641 | MSRA.GENEBODY | F | chr8:10261900-10262222 | N_Shore |
| cg16145834 | chr6:33282321 | TAPBP.TSS200 | R | chr6:33282855-33283196 | N_Shore |
| cg01654446 | chr6:33280476 | TAPBP.GENEBODY | R | chr6:33281112-33281827 | N_Shore |
| cg15396570 | chr1:85931161 | DDAH1.TSS1500 | F | chr1:85929940-85931168 | Island |
| cg17637107 | chr8:20054583 | ATP6V1B2.TSS200 | R | chr8:20054546-20055027 | Island |
| cg22692213 | chr12:133533186 | ZNF605.TSS1500 | F | chr12:133532642-133532979 | S_Shore |
| cg23596678 | chr17:79793000 | DYSFIP1.TSS200 | R | chr17:79790530-79791553 | S_Shore |
| cg21221712 | chr15:86132153 | AKAP13.GENEBODY | F | | |
| cg26130023 | chr18:45453609 | SMAD2.UTR5 | R | chr18:45456484-45458024 | N_Shelf |
| cg26539941 | chr2:220306470 | SPEG.GENEBODY | R | chr2:220306855-220307096 | N_Shore |
| cg03466032 | chr17:39968802 | FKBP10.TSS200 | R | chr17:39967407-39968604 | S_Shore |
| cg22862003 | chr21:42797588 | MX1.TSS1500 | F | chr21:42798146-42798884 | N_Shore |
| cg05801879 | chr16:84101832 | MBTPS1.GENEBODY | F | | |
| cg15040499 | chr17:35861263 | DUSP14.UTR5 | R | | |
| cg04420932 | chr11:67195621 | RPS6KB2.TSS1500 | F | chr11:67195800-67196190 | N_Shore |

TABLE 4
Top 1000 methylation sites associated with the SCLC-P subtype

| Methylation Site | Position (GRCh 37) | Gene | Strand | CpG Island Name | Relation to CpG Island |
|---|---|---|---|---|---|
| cg05204104 | chr2: 235403141 | ARL4C.UTR3 | R | chr2: 235404502- | N_Shore |
| | | | | 235406541 | |
| cg02367316 | chr7: 39125198 | POU6F2.GENEBODY | F | | |
| cg10761558 | chr1: 54842205 | SSBP3.GENEBODY | F | | |
| cg00234370 | chr1: 36021674 | KIAA0319L.UTR5 | F | chr1: 36022680-36023834 | N_Shore |
| cg16427983 | chr12: 123453858 | ABCB9.UTR5 | F | chr12: 123450868- | S_Shelf |
| | | | | 123451207 | |
| cg05942111 | chr12: 65044027 | RASSF3.GENEBODY | R | | |
| cg07120097 | chr14: 37257605 | SLC25A21.GENEBODY | R | | |
| cg10536786 | chr1: 7550036 | CAMTA1.GENEBODY | R | | |
| cg06779248 | chr7: 95458266 | DYNC1I1.GENEBODY | R | | |
| cg18831409 | chr6: 10585851 | GCNT2.GENEBODY | F | | |
| cg05264223 | chr15: 70126776 | C15orf50.TSS1500 | F | | |
| cg04765195 | chr6: 36209753 | PNPLA1.TSS1500 | F | | |
| cg14795528 | chr7: 157451260 | PTPRN2.GENEBODY | F | chr7: 157453774- | N_Shelf |
| | | | | 157454208 | |
| cg04628438 | chr6: 10585889 | GCNT2.GENEBODY | F | | |
| cg26216243 | chr6: 49495283 | GLYATL3.UTR3 | F | | |
| cg11964773 | chr1: 14924077 | KIAA1026.TSS1500 | F | chr1: 14924610-14925993 | N_Shore |
| cg13314260 | chr5: 141349631 | RNF14.UTR5 | R | chr5: 141348531- | S_Shore |
| | | | | 141349104 | |
| cg18314778 | chr7: 157468321 | PTPRN2.GENEBODY | F | | |
| cg18798446 | chr12: 103862780 | C12orf42.GENEBODY | F | | |
| cg09824889 | chr3: 121311075 | FBXO40.TSS1500 | R | | |
| cg19239304 | chr2: 238621429 | LRRFIP1.GENEBODY | R | | |
| cg13302670 | chr7: 44261293 | CAMK2B.GENEBODY | F | chr7: 44259669-44259923 | S_Shore |
| cg12108651 | chr7: 158193742 | PTPRN2.GENEBODY | F | | |
| cg10690467 | chr12: 70047752 | BEST3.UTR3 | R | | |
| cg19713140 | chr7: 157584994 | PTPRN2.GENEBODY | F | chr7: 157586711- | N_Shore |
| | | | | 157587112 | |
| cg21060736 | chr15: 73618666 | HCN4.GENEBODY | F | | |
| cg13356427 | chr1: 6520354 | ESPN.UTR3 | F | chr1: 6520584-6521955 | N_Shore |
| cg14157527 | chr9: 132937709 | FREQ.GENEBODY | R | chr9: 132934213- | S_Shelf |
| | | | | 132934483 | |
| cg07633435 | chr5: 152869009 | GRIA1.TSS1500 | F | | |
| cg20129210 | chr13: 100547192 | CLYBL.UTR3 | R | chr13: 100547172- | Island |
| | | | | 100547431 | |
| cg00970752 | chr1: 15711443 | FHAD1.GENEBODY | R | | |
| cg02746771 | chr10: 527872 | DIP2C.GENEBODY | R | chr10: 525719-526041 | S_Shore |
| cg21177112 | chr12: 63193647 | PPM1H.GENEBODY | F | chr12: 63195657-63195909 | N_Shelf |
| cg21781979 | chr13: 100547292 | CLYBL.UTR3 | F | chr13: 100547172- | Island |
| | | | | 100547431 | |
| cg21963703 | chr5: 140350766 | PCDHA7.GENEBODY | R | chr5: 140346105- | S_Shelf |
| | | | | 140346931 | |
| cg18127395 | chr7: 95433546 | DYNC1I1.UTR5 | F | | |
| cg22352709 | chr3: 195446859 | MUC20.TSS1500 | R | | |
| cg07645286 | chr2: 173604634 | RAPGEF4.GENEBODY | R | chr2: 173600281- | S_Shelf |
| | | | | 173600970 | |
| cg06923861 | chr11: 117355376 | DSCAML1.GENEBODY | F | chr11: 117352691- | S_Shelf |
| | | | | 117352961 | |
| cg19680850 | chr1: 37943106 | ZC3H12A.GENEBODY | F | chr1: 37939723-37940650 | S_Shelf |
| cg22226592 | chr1: 109439213 | GPSM2.GENEBODY | F | | |
| cg04479757 | chr22: 26951319 | MIR548J.TSS200 | R | | |
| cg07170253 | chr12: 48119903 | P11.TSS1500 | R | | |
| cg06400704 | chr1: 240256644 | FMN2.EXON1 | F | chr1: 240254959- | Island |
| | | | | 240257063 | |
| cg19786648 | chr17: 71824806 | C17orf54.TSS200 | R | | |
| cg13284574 | chr1: 6519923 | ESPN.GENEBODY | F | chr1: 6520584-6521955 | N_Shore |
| cg04415689 | chr2: 242128250 | ANO7.GENEBODY | F | | |
| cg09833249 | chr12: 103312819 | PAH.TSS1500 | R | chr12: 103311040- | S_Shore |
| | | | | 103311253 | |
| cg11545871 | chr6: 33679895 | C6orf125.TSS1500 | R | chr6: 33679158-33679701 | S_Shore |
| cg22504528 | chr22: 26951369 | MIR548J.TSS200 | F | | |
| cg16832421 | chr2: 122229878 | CLASP1.GENEBODY | F | | |
| cg16681516 | chr19: 46297285 | DMWD.TSS1500 | R | chr19: 46295855-46296347 | S_Shore |
| cg12668122 | chr3: 133094221 | TMEM108.GENEBODY | R | | |
| cg26534460 | chr2: 215896959 | ABCA12.TSS200 | F | | |
| cg14260458 | chr7: 97620123 | OCM2.TSS1500 | R | | |
| cg17852245 | chr5: 153688187 | GALNT10.GENEBODY | F | | |
| cg20141509 | chr1: 10781431 | CASZ1.UTR5 | F | | |
| cg25438492 | chr6: 33079692 | HLA-DPB2.TSS1500 | F | | |
| cg20831413 | chr11: 72460551 | ARAP1.UTR5 | R | chr11: 72463092-72463717 | N_Shelf |
| cg02394998 | chr7: 157334850 | PTPRN2.GENEBODY | R | | |
| cg12296860 | chr11: 46409428 | CHRM4.TSS1500 | R | chr11: 46410921-46414687 | N_Shore |
| cg21055933 | chr14: 64682702 | SYNE2.EXON1 | F | | |
| cg00959749 | chr1: 54302681 | TMEM48.GENEBODY | F | chr1: 54303758-54304262 | N_Shore |
| cg06665622 | chr6: 46293571 | RCAN2.TSS200 | F | | |
| cg11243251 | chr6: 36209708 | PNPLA1.TSS1500 | R | | |
| cg05554594 | chr17: 80584498 | WDR45L.GENEBODY | F | | |
| cg02565999 | chr13: 95003984 | GPC6.GENEBODY | F | | |
| cg07065666 | chr6: 167034653 | RPS6KA2.GENEBODY | R | chr6: 167032330- | S_Shelf |
| | | | | 167032582 | |
| cg19599803 | chr1: 156722676 | HDGF.TSS1500 | F | chr1: 156720747- | S_Shore |
| | | | | 156722069 | |
| cg23365482 | chr15: 40764195 | CHST14.EXON1 | F | chr15: 40763162-40764196 | Island |
| cg07920930 | chr7: 158381884 | PTPRN2.TSS1500 | R | chr7: 158379328- | S_Shore |
| | | | | 158381221 | |
| cg12817389 | chr8: 62602522 | ASPH.GENEBODY | R | | |
| cg15787636 | chr17: 80866113 | TBCD.GENEBODY | R | chr17: 80863327-80863743 | S_Shelf |
| cg18535456 | chr12: 112203801 | ALDH2.TSS1500 | R | chr12: 112204498- | N_Shore |
| | | | | 112204979 | |
| cg14703553 | chr17: 70663493 | SLC39A11.GENEBODY | R | | |
| cg19589800 | chr12: 93820263 | UBE2N.GENEBODY | F | | |
| cg07985864 | chr1: 10757892 | CASZ1.UTR5 | R | chr1: 10757891-10758252 | Island |
| cg23261073 | chr1: 9880771 | CLSTN1.GENEBODY | R | chr1: 9883011-9884667 | N_Shelf |
| cg03732014 | chr21: 30676184 | BACH1.UTR5 | F | | |
| cg16595667 | chr14: 77567533 | KIAA1737.UTR5 | F | chr14: 77563965-77564697 | S_Shelf |
| cg27630771 | chr1: 150533123 | ADAMTSL4.UTR3 | R | chr1: 150534075- | N_Shore |
| | | | | 150534301 | |
| cg07610192 | chr2: 131130423 | PTPN18.GENEBODY | R | chr2: 131129718- | Island |
| | | | | 131130511 | |
| cg15055890 | chr3: 11747650 | VGLL4.UTR5 | R | chr3: 11746577-11746824 | S_Shore |
| cg21146273 | chr5: 152869193 | GRIA1.TSS1500 | F | | |
| cg14677615 | chr17: 27315628 | SEZ6.GENEBODY | R | chr17: 27312969-27313283 | S_Shelf |
| cg14946113 | chr22: 44220872 | SULT4A1.UTR3 | F | | |
| cg17930201 | chr1: 155828174 | SYT11.TSS1500 | R | chr1: 155829109- | N_Shore |
| | | | | 155829431 | |
| cg06286135 | chr7: 157511479 | PTPRN2.GENEBODY | F | | |
| cg03442858 | chr1: 14924535 | KIAA1026.TSS1500 | F | chr1: 14924610-14925993 | N_Shore |
| cg07686441 | chr15: 52570517 | MIR1266.TSS1500 | R | | |
| cg18090068 | chr3: 55693316 | C3orf51.GENEBODY | R | | |
| cg14278148 | chr9: 114663626 | UGCG.GENEBODY | F | chr9: 114658618- | S_Shelf |
| | | | | 114659773 | |
| cg12575928 | chr11: 22687660 | GAS2.TSS1500 | R | chr11: 22688285-22688617 | N_Shore |
| cg25368586 | chr13: 111561018 | ANKRD10.GENEBODY | F | | |
| cg08976117 | chr3: 57064983 | ARHGEF3.GENEBODY | R | | |
| cg27569829 | chr18: 60984485 | BCL2.GENEBODY | R | chr18: 60985503-60985741 | N_Shore |
| cg15472885 | chr3: 71056608 | FOXP1.GENEBODY | R | | |
| cg14962159 | chr13: 37441585 | SMAD9.GENEBODY | F | | |
| cg14398557 | chr20: 1095840 | PSMF1.UTR5 | R | chr20: 1099078-1099561 | N_Shelf |
| cg10288437 | chr6: 158432894 | SYNJ2.GENEBODY | F | | |
| cg20976787 | chr6: 43064460 | PTK7.GENEBODY | R | | |
| cg10294732 | chr4: 41614397 | LIMCH1.GENEBODY | F | | |
| cg19314992 | chr11: 76614479 | ACER3.GENEBODY | R | | |
| cg16125375 | chr5: 146298512 | PPP2R2B.UTR5 | R | | |
| cg13663667 | chr19: 4180236 | SIRT6.GENEBODY | F | chr19: 4182259-4182803 | N_Shelf |
| cg02711899 | chr6: 42410890 | TRERF1.UTR5 | R | | |
| cg13127741 | chr20: 31331821 | COMMD7.TSS200 | F | chr20: 31330957-31331410 | S_Shore |
| cg08352439 | chr7: 55637123 | VOPP1.GENEBODY | F | chr7: 55639277-55640599 | N_Shelf |
| cg04528038 | chr16: 21171097 | TMEM159.UTR5 | F | chr16: 21169810-21170278 | S_Shore |
| cg06342490 | chr2: 42795193 | MTA3.TSS1500 | F | chr2: 42795847-42796230 | N_Shore |
| cg21574244 | chr15: 42568411 | GANC.GENEBODY | F | chr15: 42565452-42565791 | S_Shelf |
| cg00551589 | chr16: 2626391 | PDPK1.GENEBODY | R | chr16: 2627134-2627399 | N_Shore |
| cg17461336 | chr7: 99441559 | CYP3A43.GENEBODY | R | | |
| cg26207423 | chr8: 28787952 | HMBOX1.UTR5 | R | | |
| cg01489441 | chr16: 1764111 | MAPK8IP3.GENEBODY | R | | |
| cg27434467 | chr10: 101686326 | NCRNA00093.TSS1500 | F | | |
| cg26245086 | chr4: 87860957 | AFF1.UTR5 | F | chr4: 87855873-87857204 | S_Shelf |
| cg07696516 | chr4: 41614958 | LIMCH1.EXON1 | F | | |
| cg16457351 | chr5: 176297315 | UNC5A.GENEBODY | R | chr5: 176296541- | S_Shore |
| | | | | 176296933 | |
| cg01372366 | chr11: 48041783 | PTPRJ.GENEBODY | F | | |
| cg16039867 | chr1: 47023134 | MKNK1.GENEBODY | R | | |
| cg23615892 | chr1: 215825505 | USH2A.GENEBODY | R | | |
| cg13763232 | chr3: 14443428 | SLC6A6.TSS1500 | R | chr3: 14443629-14444577 | N_Shore |
| cg00957901 | chr4: 41614842 | LIMCH1.TSS200 | R | | |
| cg01794926 | chr15: 92470387 | SLCO3A1.GENEBODY | R | | |
| cg16544220 | chr6: 33095030 | HLA-DPB2.GENEBODY | F | | |
| cg00718444 | chr8: 145685564 | CYHR1.GENEBODY | F | chr8: 145687389- | N_Shore |
| | | | | 145687880 | |
| cg03227338 | chr11: 46998965 | C11orf49.GENEBODY | F | | |
| cg13491737 | chr13: 111295728 | CARS2.GENEBODY | R | chr13: 111294675- | S_Shore |
| | | | | 111294900 | |
| cg21579399 | chr2: 11890700 | LPIN1.UTR5 | F | chr2: 11886249-11887207 | S_Shelf |
| cg00249974 | chr1: 156254149 | TMEM79.EXON1 | R | | |
| cg15585294 | chr11: 22687641 | GAS2.TSS1500 | R | chr11: 22688285-22688617 | N_Shore |
| cg22340762 | chr1: 183823538 | RGL1.GENEBODY | F | | |
| cg00408605 | chr8: 141466079 | TRAPPC9.UTR5 | R | chr8: 141467218- | N_Shore |
| | | | | 141467927 | |
| cg00600617 | chr6: 107221887 | LOC553137.GENEBODY | F | | |
| cg00618183 | chr17: 75143600 | SEC14L1.GENEBODY | R | | |
| cg00625110 | chr16: 53741731 | FTO.GENEBODY | R | chr16: 53737617-53738235 | S_Shelf |
| cg00703885 | chr2: 128440247 | LIMS2.TSS1500 | F | | |
| cg00704369 | chr7: 81996559 | CACNA2D1.GENEBODY | R | | |
| cg00706536 | chr17: 40609826 | ATP6VOA1.TSS1500 | R | chr17: 40610746-40611032 | N_Shore |
| cg01149239 | chr3: 196746085 | MFI2.UTR3 | F | | |
| cg01307483 | chr7: 129254604 | NRF1.UTR5 | F | chr7: 129251237- | S_Shelf |
| | | | | 129252441 | |
| cg01580044 | chr1: 114300656 | PHTF1.GENEBODY | R | chr1: 114301514- | N_Shore |
| | | | | 114302272 | |
| cg01626895 | chr12: 116425790 | MED13L.GENEBODY | R | | |
| cg01653080 | chr14: 104205041 | PPP1R13B.GENEBODY | F | chr14: 104205070- | N_Shore |
| | | | | 104205329 | |
| cg01715088 | chr1: 6520772 | ESPN.UTR3 | R | chr1: 6520584-6521955 | Island |
| cg02220284 | chr3: 71013485 | FOXP1.GENEBODY | F | | |
| cg02253499 | chr3: 12602046 | MKRN2.GENEBODY | F | chr3: 12598391-12599051 | S_Shelf |
| cg02330721 | chr16: 1764231 | MAPK8IP3.GENEBODY | R | | |
| cg02519218 | chr12: 133424286 | CHFR.GENEBODY | R | | |
| cg02793948 | chr11: 68095662 | LRP5.GENEBODY | F | | |
| cg03075214 | chr11: 64867914 | C11orf2.GENEBODY | R | chr11: 64863698-64863980 | S_Shelf |
| cg03147598 | chr2: 131104160 | IMP4.UTR3 | F | chr2: 131099469- | S_Shelf |
| | | | | 131100863 | |
| cg03264729 | chr11: 72347339 | PDE2A.GENEBODY | R | | |
| cg03803789 | chr11: 111854003 | DIXDC1.GENEBODY | R | | |
| cg04390667 | chr11: 46624867 | HARBI1.UTR3 | R | | |
| cg05001389 | chr9: 135853034 | GFI1B.TSS1500 | R | | |
| cg05661060 | chr1: 44074454 | PTPRF.GENEBODY | F | chr1: 44070892-44071142 | S_Shelf |
| cg06206957 | chr7: 33080615 | NT5C3.TSS200 | R | | |
| cg06604199 | chr20: 31331973 | COMMD7.TSS200 | R | chr20: 31330957-31331410 | S_Shore |
| cg06669752 | chr5: 138534431 | SIL1.TSS1500 | R | chr5: 138533870- | S_Shore |
| | | | | 138534113 | |
| cg06832246 | chr11: 93224210 | C11orf75.UTR5 | R | | |
| cg06839854 | chr2: 10536115 | HPCAL1.UTR5 | R | | |
| cg07205154 | chr1: 176147105 | RFWD2.GENEBODY | R | | |
| cg07313835 | chr1: 167342724 | POU2F1.GENEBODY | F | | |
| cg07699978 | chr12: 63194001 | PPM1H.GENEBODY | R | chr12: 63195657-63195909 | N_Shore |
| cg07859138 | chrX: 118751995 | SEPT6.UTR3 | F | | |
| cg07956003 | chr7: 95433460 | DYNC1I1.UTR5 | F | | |
| cg08101329 | chr17: 4884631 | CAMTA2.GENEBODY | R | | |
| cg08238265 | chr14: 64682912 | SYNE2.EXON1 | R | | |
| cg08781755 | chr2: 44172074 | LRPPRC.GENEBODY | F | | |
| cg09169413 | chr12: 48103670 | P11.UTR3 | F | chr12: 48099317-48099905 | S_Shelf |
| cg10386431 | chr1: 77879031 | AK5.GENEBODY | F | | |
| cg10887937 | chr12: 112203795 | ALDH2.TSS1500 | R | chr12: 112204498- | N_Shore |
| | | | | 112204979 | |
| cg11019743 | chr6: 168267395 | MLLT4.GENEBODY | F | | |
| cg11132582 | chr1: 167217689 | POU2F1.UTR5 | F | | |
| cg11447040 | chr20: 37667339 | DHX35.UTR3 | R | | |
| cg11701604 | chr11: 36095771 | LDLRAD3.GENEBODY | F | | |
| cg13146674 | chr11: 68095623 | LRP5.GENEBODY | R | | |
| cg13556548 | chr1: 226187876 | C1orf55.TSS1500 | R | chr1: 226186803- | S_Shore |
| | | | | 226187336 | |
| cg13578134 | chr15: 75081572 | CSK.UTR5 | R | | |
| cg13619387 | chr9: 92050313 | SEMA4D.UTR5 | R | | |
| cg13676816 | chr9: 91976972 | SEMA4D.UTR3 | R | | |
| cg14211075 | chr9: 135818481 | TSC1.UTR5 | R | chr9: 135819526- | N_Shore |
| | | | | 135820529 | |
| cg14301030 | chr10: 264920 | ZMYND11.GENEBODY | R | | |
| cg14302130 | chr6: 33032830 | HLA-DPA1.UTR3 | R | | |
| cg14457284 | chr9: 135852984 | GFI1B.TSS1500 | R | | |
| cg14701925 | chr3: 128980811 | COPG.GENEBODY | F | | |
| cg14817765 | chr20: 2444494 | SNORD119.TSS1500 | F | | |
| cg15337905 | chr7: 150807508 | AGAP3.GENEBODY | R | | |
| cg15425670 | chr2: 42461109 | EML4.GENEBODY | F | | |
| cg15724534 | chr2: 211018363 | C2orf67.GENEBODY | R | | |
| cg15873449 | chr18: 12885569 | PTPN2.TSS1500 | F | chr18: 12883285-12884578 | S_Shore |
| cg16318349 | chr1: 154917307 | PBXIP1.UTR3 | R | | |
| cg16691863 | chr1: 28848173 | SNHG3-RCC1.GENEBODY | R | chr1: 28844653-28845293 | S_Shelf |
| cg17308493 | chr10: 30634469 | MTPAP.GENEBODY | F | chr10: 30637961-30638279 | N_Shelf |
| cg17355865 | chr11: 76887139 | MYO7A.GENEBODY | R | chr11: 76890783-76890986 | N_Shelf |
| cg17758792 | chr21: 37620038 | DOPEY2.GENEBODY | F | chr21: 37617467-37617872 | S_Shelf |
| cg18455249 | chr4: 56813898 | CEP135.TSS1500 | R | chr4: 56814800-56815511 | N_Shore |
| cg18872426 | chr19: 38874037 | PSMD8.UTR3 | F | chr19: 38876070-38876332 | N_Shelf |
| cg19125791 | chr2: 240265915 | HDAC4.GENEBODY | F | | |
| cg19506380 | chr8: 98740612 | MTDH.UTR3 | F | | |
| cg19918623 | chr7: 132661002 | CHCHD3.GENEBODY | R | | |
| cg20059682 | chr12: 52201821 | SCN8A.UTR3 | F | | |
| cg20467658 | chr15: 89933347 | LOC254559.GENEBODY | R | | |
| cg20497631 | chr8: 8647440 | MFHAS1.GENEBODY | R | | |
| cg20664313 | chr12: 133071297 | FBRSL1.GENEBODY | R | chr12: 133073397- | N_Shelf |
| | | | | 133073632 | |
| cg20716489 | chr3: 195591877 | TNK2.GENEBODY | R | chr3: 195594836- | N_Shelf |
| | | | | 195595494 | |
| cg21232015 | chr12: 133424221 | CHFR.GENEBODY | R | | |
| cg21289124 | chr22: 26951249 | TPST2.UTR5 | R | | |
| cg21535670 | chr8: 140798600 | TRAPPC9.GENEBODY | F | | |
| cg21877565 | chr1: 156461550 | MEF2D.UTR5 | F | | |
| cg21927971 | chr2: 17837235 | VSNL1.UTR3 | R | | |
| cg21940081 | chr2: 131103235 | IMP4.GENEBODY | R | chr2: 131099469- | S_Shelf |
| | | | | 131100863 | |
| cg22542731 | chr2: 128440274 | LIMS2.TSS1500 | F | | |
| cg23396732 | chr20: 31332170 | COMMD7.TSS1500 | F | chr20: 31330957-31331410 | S_Shore |
| cg24136932 | chr8: 22281979 | SLC39A14.GENEBODY | R | | |
| cg24440131 | chr1: 26880207 | MIR1976.TSS1500 | R | | |
| cg24495350 | chr15: 72651198 | HEXA.GENEBODY | F | | |
| cg24677222 | chr3: 140102647 | CLSTN2.GENEBODY | F | | |
| cg25601286 | chr2: 169408033 | LASS6.GENEBODY | R | | |
| cg25874150 | chr10: 34941561 | PARD3.GENEBODY | R | | |
| cg26224018 | chr1: 64942153 | CACHD1.GENEBODY | F | | |
| cg26542660 | chr4: 56813860 | CEP135.TSS1500 | R | chr4: 56814800-56815511 | N_Shore |
| cg26579713 | chr15: 65701865 | IGDCC4.GENEBODY | F | | |
| cg26797073 | chr2: 131129811 | PTPN18.GENEBODY | F | chr2: 131129718- | Island |
| | | | | 131130511 | |
| cg27053763 | chr5: 180653815 | TRIM41.GENEBODY | R | chr5: 180650099- | S_Shelf |
| | | | | 180650679 | |
| cg27222157 | chr11: 66382978 | RBM14.TSS1500 | F | chr11: 66383571-66385047 | N_Shore |
| cg27254046 | chr6: 57272398 | PRIM2.GENEBODY | F | | |
| cg00854324 | chr16: 69941269 | WWP2.GENEBODY | R | | |
| cg01099470 | chr2: 131103656 | IMP4.GENEBODY | F | chr2: 131099469- | S_Shelf |
| | | | | 131100863 | |
| cg02428072 | chr3: 169900473 | PHC3.TSS1500 | F | chr3: 169898946- | S_Shore |
| | | | | 169899626 | |
| cg03152913 | chr2: 178975712 | RBM45.TSS1500 | R | chr2: 178977078- | N_Shore |
| | | | | 178977828 | |
| cg03356778 | chr11: 120110544 | POU2F3.TSS1500 | F | chr11: 120110497- | Island |
| | | | | 120110719 | |
| cg04976330 | chr1: 31480640 | PUM1.GENEBODY | R | | |
| cg05399330 | chr1: 55464530 | BSND.TSS200 | R | chr1: 55462688-55463010 | S_Shore |
| cg06912922 | chr19: 12708229 | ZNF490.GENEBODY | F | chr19: 12707696-12708114 | S_Shore |
| cg07993367 | chr7: 149568778 | ATP6V0E2.TSS1500 | R | chr7: 149570366- | N_Shore |
| | | | | 149571272 | |
| cg08313040 | chr6: 33092243 | HLA-DPB2.GENEBODY | F | | |
| cg08320861 | chr6: 33095982 | HLA-DPB2.GENEBODY | R | | |
| cg08651894 | chr1: 154832235 | KCNN3.GENEBODY | R | | |
| cg09417011 | chr1: 54842404 | SSBP3.GENEBODY | F | | |
| cg09735908 | chr16: 88770558 | RNF166.GENEBODY | F | chr16: 88772237-88773195 | N_Shore |
| cg10833114 | chr12: 72246108 | MRS2P2.TSS1500 | F | | |
| cg12022772 | chr2: 131130468 | PTPN18.GENEBODY | R | chr2: 131129718- | Island |
| | | | | 131130511 | |
| cg12598235 | chr15: 89921182 | LOC254559.TSS200 | R | chr15: 89920793-89922768 | Island |
| cg15109449 | chr1: 12363259 | VPS13D.GENEBODY | F | | |
| cg15618336 | chr12: 52182759 | SCN8A.GENEBODY | R | | |
| cg15939973 | chr17: 76419841 | DNAH17.UTR3 | F | chr17: 76421441-76421766 | N_Shore |
| cg17301216 | chr15: 89920348 | LOC254559.TSS1500 | R | chr15: 89920793-89922768 | N_Shore |
| cg17730176 | chr17: 27404126 | TIAF1.TSS1500 | R | chr17: 27406753-27408059 | N_Shelf |
| cg18942985 | chr2: 128523110 | WDR33.GENEBODY | R | | |
| cg19465165 | chr2: 131130103 | PTPN18.GENEBODY | R | chr2: 131129718- | Island |
| | | | | 131130511 | |
| cg19856701 | chr17: 29549474 | NF1.UTR3 | F | | |
| cg20886791 | chr6: 33096055 | HLA-DPB2.GENEBODY | R | | |
| cg22158248 | chr12: 112204136 | ALDH2.TSS1500 | R | chr12: 112204498- | N_Shore |
| | | | | 112204979 | |
| cg26306289 | chr7: 33080571 | NT5C3.TSS200 | F | | |
| cg27627524 | chr8: 131325625 | ASAP1.GENEBODY | R | | |
| cg00848167 | chr15: 89921898 | LOC254559.GENEBODY | F | chr15: 89920793-89922768 | Island |
| cg01227084 | chr19: 2774520 | SGTA.UTR5 | R | chr19: 2774196-2774550 | Island |
| cg01685549 | chr6: 33092097 | HLA-DPB2.GENEBODY | R | | |
| cg01767053 | chr16: 85678884 | KIAA0182.UTR5 | R | chr16: 85674843-85675192 | S_Shelf |
| cg01951712 | chr6: 33081535 | HLA-DPB2.GENEBODY | F | chr6: 33084740-33084995 | N_Shelf |
| cg02388468 | chr6: 33095664 | HLA-DPB2.GENEBODY | F | | |
| cg02470179 | chr3: 31580476 | STT3B.GENEBODY | F | | |
| cg03766449 | chr15: 89920918 | LOC254559.TSS1500 | F | chr15: 89920793-89922768 | Island |
| cg03917138 | chr17: 70120182 | SOX9.GENEBODY | F | chr17: 70120139-70120442 | Island |
| cg04918708 | chr1: 204057900 | SOX13.UTR5 | F | | |
| cg05427639 | chr7: 33080496 | NT5C3.EXON1 | R | | |
| cg06517794 | chr1: 78180274 | USP33.GENEBODY | R | | |
| cg07586345 | chr19: 2774332 | SGTA.UTR5 | R | chr19: 2774196-2774550 | Island |
| cg09787021 | chr1: 25988486 | MAN1C1.GENEBODY | R | | |
| cg09825327 | chr1: 211503219 | TRAF5.UTR5 | F | chr1: 211500151- | S_Shelf |
| | | | | 211500636 | |
| cg10327769 | chr17: 43340243 | LOC100133991.GENEBODY | F | chr17: 43339124-43339832 | S_Shore |
| cg10657965 | chr7: 101888125 | CUX1.GENEBODY | R | chr7: 101891665- | N_Shelf |
| | | | | 101892634 | |
| cg11786476 | chr6: 33096738 | HLA-DPB2.GENEBODY | R | | |
| cg12406391 | chr1: 6520194 | ESPN.GENEBODY | F | chr1: 6520584-6521955 | N_Shore |
| cg12873454 | chr1: 14924611 | KIAA1026.TSS1500 | R | chr1: 14924610-14925993 | Island |
| cg13077784 | chr1: 201706363 | NAV1.GENEBODY | R | chr1: 201708787- | N_Shelf |
| | | | | 201709429 | |
| cg13977526 | chr9: 32384244 | ACO1.TSS1500 | F | chr9: 32384420-32384911 | N_Shore |
| cg15019001 | chr6: 33096303 | HLA-DPB2.GENEBODY | R | | |
| cg15826519 | chr1: 19808575 | CAPZB.GENEBODY | F | chr1: 19811703-19812644 | N_Shelf |
| cg16072780 | chr11: 93453897 | SCARNA9.TSS1500 | F | | |
| cg16205850 | chr15: 75118021 | LMAN1L.UTR3 | R | | |
| cg18109571 | chr6: 127835189 | C6orf174.GENEBODY | F | chr6: 127835928- | N_Shore |
| | | | | 127836255 | |
| cg18198143 | chr6: 33094306 | HLA-DPB2.GENEBODY | F | | |
| cg18474502 | chr10: 124777194 | ACADSB.GENEBODY | R | | |
| cg18680328 | chr10: 123868970 | TACC2.GENEBODY | R | chr10: 123872119- | N_Shelf |
| | | | | 123873462 | |
| cg19046783 | chr7: 99382086 | CYP3A4.TSS1500 | R | | |
| cg20314331 | chr2: 131129567 | PTPN18.GENEBODY | R | chr2: 131129718- | N_Shore |
| | | | | 131130511 | |
| cg20567355 | chr8: 52754434 | PCMTD1.GENEBODY | R | | |
| cg21516291 | chr20: 44979100 | SLC35C2.GENEBODY | F | | |
| cg21955534 | chrX: 15525514 | BMX.EXON1 | F | | |
| cg22052010 | chr20: 33541934 | GSS.UTR5 | R | chr20: 33543082-33543846 | N_Shore |
| cg22888311 | chr12: 80076443 | PAWR.GENEBODY | F | | |
| cg23538755 | chr19: 2774549 | SGTA.UTR5 | F | chr19: 2774196-2774550 | Island |
| cg24156613 | chrX: 39950501 | BCOR.UTR5 | R | chrX: 39950899-39951665 | N_Shore |
| cg24180219 | chr10: 105239190 | CALHM3.TSS200 | R | | |
| cg25064331 | chr7: 33080500 | NT5C3.EXON1 | R | | |
| cg25474399 | chr18: 3067624 | MYOM1.GENEBODY | F | chr18: 3067297-3067533 | S_Shore |
| cg25759507 | chr11: 12008553 | DKK3.GENEBODY | R | | |
| cg26086731 | chr3: 169900468 | PHC3.TSS1500 | F | chr3: 169898946- | S_Shore |
| | | | | 169899626 | |
| cg26469011 | chr10: 75886930 | AP3M1.GENEBODY | F | | |
| cg26536240 | chr16: 89509760 | ANKRD11.UTR5 | R | | |
| cg00350405 | chr2: 128244940 | IWS1.GENEBODY | F | | |
| cg01541650 | chr6: 157505968 | ARID1B.GENEBODY | R | | |
| cg02061660 | chr5: 110088130 | SLC25A46.GENEBODY | F | | |
| cg02380236 | chr15: 22383188 | OR4N4.EXON1 | F | | |
| cg03233656 | chr2: 65214625 | SLC1A4.TSS1500 | F | chr2: 65215598-65217212 | N_Shore |
| cg04824189 | chr7: 80142634 | GNAT3.TSS1500 | F | | |
| cg05009111 | chr20: 58520201 | C20orf177.UTR3 | R | | |
| cg05366160 | chr6: 33039481 | HLA-DPA1.GENEBODY | F | | |
| cg06614640 | chr17: 80826697 | TBCD.GENEBODY | R | chr17: 80827970-80828316 | N_Shore |
| cg09142647 | chr16: 75674568 | KARS.GENEBODY | R | | |
| cg10094078 | chr19: 1467925 | APC2.GENEBODY | F | chr19: 1465206-1471241 | Island |
| cg10297701 | chr6: 32972902 | HLA-DOA.UTR3 | F | chr6: 32975684-32975926 | N_Shelf |
| cg10604002 | chr2: 131130131 | PTPN18.GENEBODY | R | chr2: 131129718- | Island |
| | | | | 131130511 | |
| cg10725390 | chr2: 66743000 | MEIS1.GENEBODY | F | | |
| cg12733040 | chr22: 17303493 | XKR3.TSS1500 | F | | |
| cg12748948 | chr1: 22106295 | USP48.GENEBODY | F | chr1: 22109129-22110261 | N_Shelf |
| cg12815916 | chr2: 131114432 | PTPN18.GENEBODY | F | chr2: 131113090- | S_Shore |
| | | | | 131114277 | |
| cg13726789 | chr8: 37678323 | GPR124.GENEBODY | F | | |
| cg13922121 | chr11: 19575262 | NAV2.GENEBODY | F | | |
| cg15214229 | chr20: 35275274 | SLA2.TSS1500 | R | | |
| cg16296829 | chr1: 154832535 | KCNN3.TSS200 | R | | |
| cg16431810 | chr7: 2420339 | EIF3B.UTR3 | F | chr7: 2419808-2420340 | Island |
| cg18451156 | chr12: 133424373 | CHFR.GENEBODY | R | | |
| cg18963065 | chrX: 15525321 | BMX.UTR5 | F | | |
| cg21049501 | chr17: 70120411 | SOX9.GENEBODY | F | chr17: 70120139-70120442 | Island |
| cg21121843 | chr4: 3203982 | HTT.GENEBODY | R | | |
| cg21156320 | chr12: 56112820 | BLOC1S1.GENEBODY | R | chr12: 56109798-56110298 | S_Shelf |
| cg04403809 | chr20: 44686493 | SLC12A5.UTR3 | R | chr20: 44685771-44687610 | Island |
| cg00538021 | chr16: 12192263 | SNX29.GENEBODY | R | | |
| cg00712146 | chr3: 65340538 | MAGI1.UTR3 | R | chr3: 65342203-65342884 | N_Shore |
| cg01249653 | chr6: 33095755 | HLA-DPB2.GENEBODY | F | | |
| cg03212228 | chr19: 38395481 | WDR87.UTR5 | F | chr19: 38397003-38398165 | N_Shore |
| cg03387092 | chr3: 123493248 | MYLK.GENEBODY | R | | |
| cg04117530 | chr1: 226187852 | C1orf55.TSS1500 | R | chr1: 226186803- | S_Shore |
| | | | | 226187336 | |
| cg04256886 | chr2: 25603640 | DTNB.GENEBODY | R | chr2: 25599676-25599895 | S_Shelf |
| cg04351807 | chr11: 36013639 | LDLRAD3.GENEBODY | R | | |
| cg04478905 | chr21: 37915091 | CLDN14.TSS200 | F | | |
| cg04998498 | chr11: 113725687 | USP28.GENEBODY | F | | |
| cg05613017 | chr12: 65589446 | LEMD3.GENEBODY | F | | |
| cg06234051 | chr17: 70120541 | SOX9.UTR3 | F | chr17: 70116274-70119998 | S_Shore |
| cg06532924 | chr1: 247268238 | ZNF669.TSS1500 | F | chr1: 247267179- | S_Shore |
| | | | | 247267884 | |
| cg07069844 | chr19: 1617027 | TCF3.GENEBODY | F | chr19: 1615202-1615812 | S_Shore |
| cg07202461 | chr18: 19406765 | MIB1.GENEBODY | F | | |
| cg07813851 | chr1: 7466976 | CAMTA1.GENEBODY | F | | |
| cg08088222 | chr12: 122070432 | ORAI1.GENEBODY | F | | |
| cg08948637 | chr20: 62614493 | PRPF6.GENEBODY | R | chr20: 62612300-62612787 | S_Shore |
| cg10004079 | chr17: 26372530 | NLK.GENEBODY | R | chr17: 26368586-26369133 | S_Shelf |
| cg10735599 | chr6: 33081024 | HLA-DPB2.GENEBODY | F | chr6: 33084740-33084995 | N_Shelf |
| cg10932086 | chr14: 90041348 | PRO1768.TSS1500 | F | | |
| cg12255995 | chr19: 1132537 | SBNO2.GENEBODY | R | | |
| cg12555369 | chr10: 105153066 | USMG5.UTR5 | R | chr10: 105155820- | N_Shelf |
| | | | | 105156484 | |
| cg12577003 | chr7: 2261337 | MAD1L1.GENEBODY | R | | |
| cg12582616 | chr6: 15427489 | JARID2.GENEBODY | F | | |
| cg13058710 | chr17: 70119609 | SOX9.GENEBODY | F | chr17: 70116274-70119998 | Island |
| cg13362033 | chr1: 7018949 | CAMTA1.GENEBODY | F | chr1: 7022001-7022308 | N_Shelf |
| cg13726166 | chr17: 27403888 | TIAF1.TSS1500 | F | chr17: 27406753-27408059 | N_Shelf |
| cg13738075 | chr13: 114793125 | RASA3.GENEBODY | R | | |
| cg14373068 | chr11: 1537765 | HCCA2.GENEBODY | F | chr11: 1536729-1536953 | S_Shore |
| cg14521421 | chr11: 8862019 | ST5.UTR5 | R | | |
| cg14762436 | chr7: 24917750 | OSBPL3.GENEBODY | F | | |
| cg17223866 | chr1: 44794158 | ERI3.GENEBODY | F | | |
| cg18236734 | chr3: 183817931 | HTR3E.TSS200 | F | | |
| cg18714586 | chr13: 30831499 | KATNAL1.GENEBODY | R | | |
| cg19232395 | chr11: 8550221 | STK33.UTR5 | R | | |
| cg19251161 | chr10: 106094166 | ITPRIP.TSS1500 | R | chr10: 106097595- | N_Shelf |
| | | | | 106098556 | |
| cg19490899 | chr6: 52855923 | GSTA4.GENEBODY | R | chr6: 52859565-52859767 | N_Shelf |
| cg19644145 | chr7: 21488742 | SP4.GENEBODY | F | | |
| cg20288892 | chr7: 82072332 | CACNA2D1.GENEBODY | R | chr7: 82072021-82073520 | Island |
| cg20706496 | chr6: 112084071 | FYN.UTR5 | R | | |
| cg22235258 | chr11: 34675402 | EHF.GENEBODY | F | | |
| cg22736267 | chr7: 148841524 | ZNF398.UTR5 | F | chr7: 148844254- | N_Shelf |
| | | | | 148845556 | |
| cg23025804 | chr10: 29811659 | SVIL.GENEBODY | F | | |
| cg23794671 | chr6: 32977580 | HLA-DOA.TSS200 | F | chr6: 32975684-32975926 | S_Shore |
| cg23998903 | chr2: 209048830 | C2orf80.GENEBODY | F | | |
| cg24223075 | chr11: 119137279 | CBL.GENEBODY | F | | |
| cg24321688 | chr19: 39393347 | NFKBIB.GENEBODY | F | chr19: 39395881-39396106 | N_Shelf |
| cg24904278 | chr4: 3514709 | LRPAP1.GENEBODY | F | chr4: 3514537-3514848 | Island |
| cg24918798 | chr2: 103432687 | TMEM182.UTR3 | R | | |
| cg25380514 | chr3: 169900418 | PHC3.TSS1500 | F | chr3: 169898946- | S_Shore |
| | | | | 169899626 | |
| cg25649889 | chr2: 171196296 | MYO3B.GENEBODY | F | | |
| cg25681122 | chr3: 194832600 | C3orf21.GENEBODY | F | | |
| cg25848827 | chr1: 7550111 | CAMTA1.GENEBODY | R | | |
| cg25977504 | chr1: 247979157 | OR14A16.TSS200 | R | | |
| cg00425764 | chr16: 15949616 | MYH11.UTR5 | R | chr16: 15950839-15951266 | N_Shore |
| cg00863378 | chr16: 56549757 | BBS2.GENEBODY | R | chr16: 56553529-56554075 | N_Shelf |
| cg00946598 | chr1: 44117093 | KDM4A.UTR5 | F | chr1: 44115616-44116309 | S_Shore |
| cg01140395 | chr15: 52570565 | MIR1266.TSS1500 | F | | |
| cg01418162 | chr1: 204891567 | NFASC.UTR5 | R | | |
| cg01708014 | chr14: 51299002 | NIN.TSS1500 | R | chr14: 51296727-51298477 | S_Shore |
| cg01772439 | chr5: 149012102 | FLJ41603.UTR3 | R | | |
| cg01859360 | chr17: 26990458 | SUPT6H.UTR5 | R | chr17: 26988721-26989391 | S_Shore |
| cg01891209 | chr1: 44401222 | ARTN.TSS1500 | R | chr1: 44401757-44402423 | N_Shore |
| cg02362718 | chr11: 133942581 | JAM3.GENEBODY | F | chr11: 133938850- | S_Shelf |
| | | | | 133939681 | |
| cg02632809 | chr7: 5336811 | SLC29A4.GENEBODY | R | chr7: 5336513-5336894 | Island |
| cg04022912 | chr16: 24864984 | SLC5A11.UTR5 | F | | |
| cg04153082 | chr8: 48325740 | KIAA0146.GENEBODY | F | | |
| cg04272613 | chr14: 59721173 | DAAM1.UTR5 | F | | |
| cg04694619 | chr2: 242127759 | ANO7.TSS200 | F | | |
| cg05995528 | chr6: 33036495 | HLA-DPA1.GENEBODY | F | | |
| cg06821199 | chr4: 779230 | CPLX1.UTR3 | F | chr4: 778661-780592 | Island |
| cg06985721 | chr1: 55464653 | BSND.EXON1 | F | chr1: 55462688-55463010 | S_Shore |
| cg08961287 | chr15: 89919993 | LOC254559.TSS1500 | F | chr15: 89920793-89922768 | N_Shore |
| cg09561830 | chr13: 100007116 | UBAC2.GENEBODY | R | | |
| cg10042298 | chr11: 78024311 | GAB2.GENEBODY | R | | |
| cg10471574 | chr17: 70120318 | SOX9.GENEBODY | F | chr17: 70120139-70120442 | Island |
| cg10803321 | chr1: 50629523 | ELAVL4.GENEBODY | R | | |
| cg10818986 | chr1: 248083146 | OR2T8.TSS1500 | R | | |
| cg11131599 | chr1: 204224211 | PLEKHA6.GENEBODY | F | chr1: 204226544- | N_Shelf |
| | | | | 204226908 | |
| cg12338214 | chr10: 133783113 | BNIP3.GENEBODY | R | | |
| cg12499542 | chr12: 53808156 | SP1.UTR3 | R | | |
| cg12832726 | chr11: 68096165 | LRP5.GENEBODY | F | | |
| cg13375479 | chr7: 133984347 | SLC35B4.GENEBODY | F | | |
| cg13551330 | chr9: 32388149 | ACO1.UTR5 | R | chr9: 32384420-32384911 | S_Shelf |
| cg13696135 | chr12: 49396782 | PRKAG1.GENEBODY | R | chr12: 49393139-49393361 | S_Shelf |
| cg14200678 | chr9: 123380343 | MEGF9.GENEBODY | F | | |
| cg14373797 | chr6: 33053576 | HLA-DPB1.GENEBODY | R | | |
| cg14880894 | chr4: 78697859 | CNOT6L.GENEBODY | R | | |
| cg15661715 | chr7: 151401947 | PRKAG2.GENEBODY | F | | |
| cg15955522 | chr5: 143553445 | KCTD16.UTR5 | R | chr5: 143549939- | S_Shelf |
| | | | | 143550333 | |
| cg16083838 | chr11: 2889809 | KCNQ1DN.TSS1500 | R | chr11: 2890388-2891337 | N_Shore |
| cg16189995 | chr2: 226330770 | KIAA1486.GENEBODY | R | | |
| cg17491228 | chr11: 64476089 | NRXN2.GENEBODY | F | chr11: 64478843-64479598 | N_Shelf |
| cg17851748 | chr12: 83264140 | TMTC2.GENEBODY | R | | |
| cg18063824 | chr4: 54075042 | SCFD2.GENEBODY | F | | |
| cg18282011 | chr1: 1590447 | CDK11B.GENEBODY | F | chr1: 1590073-1590942 | Island |
| cg19083007 | chr6: 46293862 | RCAN2.TSS1500 | R | | |
| cg19740679 | chr1: 21930180 | RAP1GAP.GENEBODY | R | chr1: 21930179-21930380 | Island |
| cg20535805 | chr19: 18420735 | LSM4.GENEBODY | F | chr19: 18420489-18420736 | Island |
| cg20643029 | chr21: 37915044 | CLDN14.TSS200 | F | | |
| cg21039196 | chr6: 71435620 | SMAP1.GENEBODY | F | | |
| cg21221516 | chr3: 183950129 | VWA5B2.GENEBODY | F | chr3: 183947040- | S_Shore |
| | | | | 183948801 | |
| cg21350778 | chr17: 64297603 | PRKCA.TSS1500 | R | chr17: 64298047-64299617 | N_Shore |
| cg23034373 | chr21: 37915354 | CLDN14.UTR5 | F | | |
| cg23200572 | chr6: 33096501 | HLA-DPB2.GENEBODY | R | | |
| cg24008634 | chr10: 13694895 | FRMD4A.GENEBODY | R | chr10: 13698562-13699540 | N_Shelf |
| cg24401199 | chr19: 3159786 | GNA15.GENEBODY | F | chr19: 3162841-3163161 | N_Shelf |
| cg24677302 | chr14: 75922163 | JDP2.GENEBODY | R | | |
| cg24987745 | chr2: 17819167 | VSNL1.GENEBODY | R | | |
| cg25119261 | chr6: 33081351 | HLA-DPB2.GENEBODY | R | chr6: 33084740-33084995 | N_Shelf |
| cg25243171 | chr2: 23914356 | KLHL29.GENEBODY | R | chr2: 23913308-23913569 | S_Shore |
| cg26886855 | chr8: 10694831 | PINX1.GENEBODY | R | chr8: 10697016-10697634 | N_Shelf |
| cg26949694 | chr11: 27742060 | BDNF.GENEBODY | R | chr11: 27742059-27742273 | Island |
| cg27636594 | chr7: 38767436 | VPS41.GENEBODY | R | | |
| cg01208614 | chr5: 141031443 | FCHSD1.TSS1500 | R | chr5: 141030918- | S_Shore |
| | | | | 141031177 | |
| cg02389877 | chr15: 68583600 | FEM1B.UTR3 | F | | |
| cg02882004 | chr2: 201334663 | SPATS2L.GENEBODY | R | | |
| cg04077303 | chr1: 236308094 | GPR137B.GENEBODY | R | chr1: 236305534- | S_Shore |
| | | | | 236306674 | |
| cg04576847 | chr17: 12623611 | MYOCD.GENEBODY | R | | |
| cg06879534 | chr4: 39447437 | KLB.GENEBODY | F | chr4: 39448184-39449054 | N_Shore |
| cg07010146 | chr10: 105239205 | CALHM3.TSS1500 | R | | |
| cg07304186 | chr6: 38112278 | ZFAND3.GENEBODY | R | | |
| cg08759770 | chr12: 51849767 | SLC4A8.GENEBODY | F | | |
| cg13339454 | chr2: 242127690 | ANO7.TSS1500 | F | | |
| cg14080888 | chr9: 130712990 | FAM102A.UTR5 | F | | |
| cg18477771 | chr11: 120195466 | TMEM136.TSS1500 | F | chr11: 120195805- | N_Shore |
| | | | | 120196372 | |
| cg19377250 | chr7: 100463206 | SLC12A9.GENEBODY | F | chr7: 100463758- | N_Shore |
| | | | | 100464146 | |
| cg20272287 | chr17: 19247905 | MIR1180.TSS200 | F | chr17: 19247785-19248069 | Island |
| cg21265278 | chr1: 179057067 | TOR3A.GENEBODY | F | | |
| cg23541975 | chr17: 75143184 | SEC14L1.GENEBODY | R | | |
| cg00552822 | chr15: 22368443 | LOC727924.GENEBODY | R | | |
| cg00796611 | chr1: 206603444 | SRGAP2.GENEBODY | R | | |
| cg01367339 | chr3: 44962718 | ZDHHC3.GENEBODY | R | | |
| cg01638185 | chr17: 64530027 | PRKCA.GENEBODY | R | | |
| cg01668281 | chr21: 37915281 | CLDN14.UTR5 | R | | |
| cg01715699 | chr13: 99209655 | STK24.GENEBODY | R | | |
| cg01854491 | chr8: 144815021 | FAM83H.UTR5 | F | chr8: 144815203- | N_Shore |
| | | | | 144816518 | |
| cg02246992 | chr17: 29158363 | ATAD5.TSS1500 | F | chr17: 29158878-29159423 | N_Shore |
| cg02370478 | chrX: 106241676 | MORC4.GENEBODY | R | chrX: 106242506- | N_Shore |
| | | | | 106243593 | |
| cg02544829 | chr8: 95385408 | RAD54B.GENEBODY | F | | |
| cg03332109 | chr11: 126360468 | KIRREL3.GENEBODY | F | | |
| cg04086197 | chr14: 104205071 | PPP1R13B.GENEBODY | R | chr14: 104205070- | Island |
| | | | | 104205329 | |
| cg04236786 | chr8: 41580903 | ANK1.GENEBODY | R | chr8: 41583135-41583524 | N_Shelf |
| cg04920416 | chr17: 35336293 | AATF.GENEBODY | R | | |
| cg05490757 | chr17: 77130342 | HRNBP3.UTR5 | F | chr17: 77133927-77134175 | N_Shelf |
| cg06927775 | chr17: 40609423 | ATP6VOA1.TSS1500 | R | chr17: 40610746-40611032 | N_Shore |
| cg07481360 | chr3: 196902551 | DLG1.GENEBODY | F | | |
| cg07535740 | chr19: 5915494 | CAPS.UTR3 | F | chr19: 5915007-5915301 | S_Shore |
| cg08707617 | chrX: 39944352 | BCOR.UTR5 | R | chrX: 39942756-39943419 | S_Shore |
| cg08760402 | chr6: 33036486 | HLA-DPA1.GENEBODY | R | | |
| cg09002351 | chr16: 75204945 | ZFP1.UTR3 | F | | |
| cg09203240 | chr11: 68115891 | LRP5.GENEBODY | R | chr11: 68115317-68115726 | S_Shore |
| cg10674511 | chrX: 78003091 | LPAR4.TSS200 | F | | |
| cg10808783 | chr19: 1467921 | APC2.GENEBODY | F | chr19: 1465206-1471241 | Island |
| cg11336987 | chr3: 123974913 | KALRN.GENEBODY | F | | |
| cg12700788 | chr17: 72348160 | KIF19.GENEBODY | R | chr17: 72347924-72348322 | Island |
| cg12892471 | chr12: 50616779 | LIMA1.GENEBODY | F | | |
| cg13356324 | chr1: 228332502 | GUK1.UTR5 | R | chr1: 228327508- | S_Shelf |
| | | | | 228328560 | |
| cg14353201 | chr11: 67448395 | ALDH3B2.UTR5 | F | | |
| cg15770046 | chr19: 35764710 | USF2.GENEBODY | F | chr19: 35760457-35760865 | S_Shelf |
| cg16712103 | chr1: 193150499 | B3GALT2.GENEBODY | F | | |
| cg16831185 | chr17: 29158530 | ATAD5.TSS1500 | F | chr17: 29158878-29159423 | N_Shore |
| cg17219729 | chr1: 36937845 | CSF3R.GENEBODY | F | | |
| cg17582782 | chr15: 57592438 | LOC283663.TSS200 | F | | |
| cg17649671 | chr10: 89298596 | MINPP1.GENEBODY | F | | |
| cg18106482 | chr11: 2680314 | KCNQ1.GENEBODY | R | | |
| cg18993757 | chr17: 4889754 | CAMTA2.UTR5 | F | chr17: 4890282-4890991 | N_Shore |
| cg19113599 | chr15: 99683042 | TTC23.GENEBODY | R | | |
| cg19296405 | chr19: 18368483 | KIAA1683.GENEBODY | R | chr19: 18368482-18368911 | Island |
| cg19533977 | chr17: 57719682 | CLTC.GENEBODY | F | | |
| cg19601328 | chr17: 43368729 | MAP3K14.UTR5 | R | | |
| cg19909036 | chr15: 90294776 | MESP1.TSS1500 | F | chr15: 90293221-90294733 | S_Shore |
| cg20664654 | chrX: 153169465 | AVPR2.TSS1500 | R | | |
| cg22057240 | chr5: 6755843 | POLS.UTR3 | R | chr5: 6755104-6755387 | S_Shore |
| cg22675922 | chr11: 2889840 | KCNQ1DN.TSS1500 | R | chr11: 2890388-2891337 | N_Shore |
| cg24051242 | chr11: 68096138 | LRP5.GENEBODY | R | | |
| cg24063043 | chr17: 64567021 | PRKCA.GENEBODY | R | | |
| cg24420432 | chr7: 101518619 | CUX1.GENEBODY | F | | |
| cg27050612 | chr17: 46133198 | NFE2L1.GENEBODY | F | chr17: 46136649-46136969 | N_Shelf |
| cg27064928 | chrX: 146996911 | ASFMR1.GENEBODY | F | chrX: 146993091- | S_Shelf |
| | | | | 146994200 | |
| cg01966522 | chr3: 126236504 | UROC1.EXON1 | R | | |
| cg04619304 | chr12: 24434537 | SOX5.UTR5 | R | | |
| cg05043047 | chr8: 144583910 | ZC3H3.GENEBODY | R | | |
| cg05169160 | chr2: 178484487 | TTC30A.TSS1500 | R | chr2: 178483233- | S_Shore |
| | | | | 178483783 | |
| cg05733361 | chr15: 85524456 | PDE8A.TSS1500 | R | chr15: 85523512-85525865 | Island |
| cg06895124 | chr8: 141588750 | EIF2C2.GENEBODY | R | | |
| cg07178896 | chr16: 50238554 | PAPD5.GENEBODY | F | | |
| cg09038267 | chr10: 104524861 | C10orf26.GENEBODY | F | | |
| cg10369242 | chr12: 52625486 | KRT7.TSS1500 | R | chr12: 52626793-52627577 | N_Shore |
| cg11678118 | chr6: 122935372 | PKIB.UTR5 | F | chr6: 122931153- | S_Shelf |
| | | | | 122931705 | |
| cg12100006 | chr11: 35964190 | LDLRAD3.TSS1500 | R | chr11: 35965642-35966454 | N_Shore |
| cg12738079 | chrX: 153169469 | AVPR2.TSS1500 | R | | |
| cg12745145 | chr7: 2261452 | MAD1L1.GENEBODY | F | | |
| cg13259357 | chr3: 191109928 | CCDC50.UTR3 | F | | |
| cg14356799 | chr6: 33044345 | HLA-DPB1.GENEBODY | R | | |
| cg14981137 | chr5: 50678697 | ISL1.TSS1500 | R | chr5: 50678632-50679375 | Island |
| cg16235962 | chr11: 118754507 | CXCR5.TSS200 | F | | |
| cg18907656 | chr8: 30888925 | PURG.GENEBODY | F | | |
| cg19407960 | chr6: 33094829 | HLA-DPB2.GENEBODY | F | | |
| cg20738500 | chr15: 89921156 | LOC254559.TSS200 | F | chr15: 89920793-89922768 | Island |
| cg20851325 | chrX: 153169432 | AVPR2.TSS1500 | F | | |
| cg21211730 | chr19: 45279770 | CBLC.TSS1500 | F | chr19: 45281133-45281355 | N_Shore |
| cg21608489 | chr19: 42637137 | POU2F2.TSS1500 | R | chr19: 42637277-42637684 | N_Shore |
| cg22338118 | chr7: 94536003 | PPP1R9A.TSS1500 | F | chr7: 94536849-94537477 | N_Shore |
| cg24644448 | chr2: 114383268 | RPL23AP7.GENEBODY | R | chr2: 114384589- | N_Shore |
| | | | | 114385133 | |
| cg25694790 | chr20: 17582752 | DSTN.GENEBODY | F | | |
| cg26067647 | chr19: 1420404 | DAZAP1.GENEBODY | F | chr19: 1419751-1420405 | Island |
| cg26643856 | chr6: 32977349 | HLA-DOA.EXON1 | R | chr6: 32975684-32975926 | S_Shore |
| cg27500983 | chr20: 34700201 | EPB41L1.TSS200 | R | | |
| cg00166343 | chr17: 29150100 | CRLF3.GENEBODY | R | chr17: 29151481-29151901 | N_Shore |
| cg01091565 | chr15: 90294762 | MESP1.TSS1500 | F | chr15: 90293221-90294733 | S_Shore |
| cg01755009 | chr1: 247978690 | OR14A16.EXON1 | F | | |
| cg02347367 | chr6: 109250017 | ARMC2.GENEBODY | R | | |
| cg02397571 | chr8: 28175463 | PNOC.UTR5 | F | | |
| cg06544652 | chr1: 248006259 | OR11L1.TSS1500 | R | | |
| cg08097973 | chr11: 71793051 | LRTOMT.UTR5 | R | chr11: 71791385-71791960 | S_Shore |
| cg08822075 | chr7: 26193607 | NFE2L3.GENEBODY | R | chr7: 26191794-26192757 | S_Shore |
| cg10057033 | chr8: 41737893 | ANK1.GENEBODY | F | chr8: 41733535-41733911 | S_Shelf |
| cg11301657 | chr12: 3749918 | EFCAB4B.GENEBODY | R | | |
| cg11437465 | chr6: 33036958 | HLA-DPA1.GENEBODY | F | | |
| cg12704098 | chr3: 56838665 | ARHGEF3.GENEBODY | F | chr3: 56835650-56836210 | S_Shelf |
| cg15642209 | chr17: 66290505 | ARSG.UTR5 | R | chr17: 66286541-66288532 | S_Shore |
| cg15841865 | chr7: 143173551 | TAS2R41.TSS1500 | F | | |
| cg16027403 | chr1: 247978791 | OR14A16.EXON1 | F | | |
| cg16527552 | chr19: 42816045 | TMEM145.TSS1500 | R | chr19: 42817412-42818038 | N_Shore |
| cg17138415 | chr12: 107259568 | RIC8B.GENEBODY | R | | |
| cg17395184 | chr15: 42750462 | ZFP106.TSS1500 | R | | |
| cg19333963 | chr19: 1467979 | APC2.GENEBODY | F | chr19: 1465206-1471241 | Island |
| cg19494588 | chr5: 146195103 | PPP2R2B.GENEBODY | R | | |
| cg22392325 | chr1: 7049418 | CAMTA1.GENEBODY | F | | |
| cg23746600 | chr6: 160403181 | IGF2R.GENEBODY | F | | |
| cg25019648 | chr17: 72353261 | BTBD17.GENEBODY | R | chr17: 72352827-72353798 | Island |
| cg25444002 | chr13: 102520143 | FGF14.GENEBODY | R | | |
| cg27121870 | chr11: 134257030 | B3GAT1.GENEBODY | R | chr11: 134257428- | N_Shore |
| | | | | 134257631 | |
| cg27319536 | chr5: 146260901 | PPP2R2B.UTR5 | R | chr5: 146257346- | S_Shelf |
| | | | | 146258575 | |
| cg27382459 | chr2: 145237379 | ZEB2.GENEBODY | F | | |
| cg27639080 | chr12: 49960363 | MCRS1.GENEBODY | R | chr12: 49961630-49962219 | N_Shore |
| cg00762029 | chr4: 185317438 | IRF2.GENEBODY | R | | |
| cg01217406 | chr15: 23013480 | NIPA2.GENEBODY | R | | |
| cg01220513 | chrX: 19902129 | SH3KBP1.GENEBODY | F | chrX: 19905027-19906557 | N_Shelf |
| cg01620165 | chr12: 113530915 | DTX1.GENEBODY | R | chr12: 113530914- | Island |
| | | | | 113531476 | |
| cg01708377 | chr6: 1686746 | GMDS.GENEBODY | R | | |
| cg02291620 | chr1: 233365140 | PCNXL2.GENEBODY | F | | |
| cg02394186 | chr1: 6088550 | KCNAB2.UTR5 | F | chr1: 6086244-6086494 | S_Shelf |
| cg03389720 | chr16: 8780048 | ABAT.UTR5 | R | | |
| cg04021887 | chr2: 10566989 | HPCAL1.UTR3 | R | chr2: 10566889-10567148 | Island |
| cg05058976 | chr16: 3637956 | BTBD12.GENEBODY | R | chr16: 3639092-3639306 | N_Shore |
| cg05157251 | chr18: 57566460 | PMAIP1.TSS1500 | R | chr18: 57566866-57567723 | N_Shore |
| cg05407550 | chrX: 154250784 | F8.EXON1 | R | | |
| cg05621843 | chr9: 97806397 | C9orf3.GENEBODY | F | chr9: 97807450-97807694 | N_Shore |
| cg07038822 | chr11: 125830798 | CDON.UTR3 | F | | |
| cg07507418 | chr6: 42372284 | TRERF1.UTR5 | R | | |
| cg07716663 | chr19: 42636625 | POU2F2.UTR5 | F | chr19: 42637277-42637684 | N_Shore |
| cg07768372 | chr5: 14509008 | TRIO.UTR3 | F | | |
| cg07849009 | chr7: 105663410 | FLJ23834.GENEBODY | F | | |
| cg07960154 | chr17: 37760198 | NEUROD2.UTR3 | F | chr17: 37761691-37763551 | N_Shore |
| cg08438392 | chr2: 27708644 | IFT172.GENEBODY | R | chr2: 27712554-27712842 | N_Shelf |
| cg09016768 | chr11: 118982033 | C2CD2L.GENEBODY | F | chr11: 118977878- | S_Shelf |
| | | | | 118978785 | |
| cg09640202 | chr2: 131101440 | IMP4.GENEBODY | R | chr2: 131099469- | S_Shore |
| | | | | 131100863 | |
| cg10090761 | chr19: 1468466 | APC2.GENEBODY | F | chr19: 1465206-1471241 | Island |
| cg11650039 | chrX: 15525325 | BMX.UTR5 | R | | |
| cg12110887 | chr19: 11096925 | SMARCA4.GENEBODY | R | chr19: 11098395-11098609 | N_Shore |
| cg12184624 | chr10: 105239164 | CALHM3.TSS200 | F | | |
| cg12426293 | chr18: 8612540 | RAB12.GENEBODY | F | chr18: 8608763-8610370 | S_Shelf |
| cg12858166 | chr6: 33033176 | HLA-DPA1.UTR3 | R | | |
| cg13027851 | chr2: 102433793 | MAP4K4.GENEBODY | F | | |
| cg13403121 | chr7: 148963719 | ZNF783.GENEBODY | R | chr7: 148958858- | S_Shelf |
| | | | | 148959891 | |
| cg15076824 | chr1: 159685185 | CRP.TSS1500 | F | | |
| cg15795819 | chr8: 39328433 | ADAM3A.GENEBODY | R | | |
| cg16175713 | chr6: 46617172 | CYP39A1.GENEBODY | F | chr6: 46620541-46621189 | N_Shelf |
| cg16430279 | chr1: 1266504 | TAS1R3.TSS1500 | F | chr1: 1267085-1267286 | N_Shore |
| cg18457491 | chr10: 104829517 | CNNM2.GENEBODY | R | | |
| cg18586851 | chr20: 31332353 | COMMD7.TSS1500 | R | chr20: 31330957-31331410 | S_Shore |
| cg19237600 | chr7: 99564474 | AZGP1.UTR3 | R | | |
| cg19305488 | chr19: 1468359 | APC2.GENEBODY | F | chr19: 1465206-1471241 | Island |
| cg19861842 | chr8: 22855745 | RHOBTB2.GENEBODY | R | chr8: 22856800-22857728 | N_Shore |
| cg20058937 | chr7: 5336543 | SLC29A4.GENEBODY | R | chr7: 5336513-5336894 | Island |
| cg20668952 | chr15: 75082885 | CSK.UTR5 | F | | |
| cg20927312 | chr3: 14443473 | SLC6A6.TSS1500 | R | chr3: 14443629-14444577 | N_Shore |
| cg21702431 | chr17: 19617084 | SLC47A2.GENEBODY | R | chr17: 19617083-19617424 | Island |
| cg23750365 | chr6: 33043072 | HLA-DPB1.TSS1500 | F | | |
| cg24532476 | chr12: 133424566 | CHFR.GENEBODY | F | | |
| cg24686845 | chr12: 94698752 | PLXNC1.GENEBODY | F | | |
| cg25052933 | chr6: 33052888 | HLA-DPB1.GENEBODY | F | | |
| cg25087423 | chr11: 118754535 | CXCR5.TSS200 | R | | |
| cg25255988 | chr11: 75428927 | MOGAT2.TSS200 | R | | |
| cg25901444 | chr7: 5336713 | SLC29A4.GENEBODY | R | chr7: 5336513-5336894 | Island |
| cg26074140 | chr1: 44401309 | ARTN.TSS1500 | F | chr1: 44401757-44402423 | N_Shore |
| cg26300785 | chr10: 81075492 | ZMIZ1.UTR3 | F | | |
| cg26385743 | chr2: 131101463 | IMP4.GENEBODY | R | chr2: 131099469- | S_Shore |
| | | | | 131100863 | |
| cg26455386 | chr17: 16999983 | MPRIP.GENEBODY | R | | |
| cg26576576 | chr1: 214627705 | PTPN14.GENEBODY | R | | |
| cg27134386 | chr4: 46392485 | GABRA2.TSS1500 | R | chr4: 46391903-46392572 | Island |
| cg27407935 | chr17: 17723235 | SREBF1.GENEBODY | F | chr17: 17726855-17727282 | N_Shelf |
| cg00457932 | chr15: 28145216 | OCA2.GENEBODY | F | chr15: 28147925-28148465 | N_Shelf |
| cg03402235 | chr15: 42749336 | ZFP106.EXON1 | F | | |
| cg04245374 | chr1: 168150977 | TIPRL.GENEBODY | F | chr1: 168147963- | S_Shelf |
| | | | | 168148637 | |
| cg05571310 | chr17: 72350354 | KIF19.GENEBODY | R | chr17: 72350346-72350711 | Island |
| cg06253072 | chr6: 33679850 | C6orf125.TSS1500 | F | chr6: 33679158-33679701 | S_Shore |
| cg07209550 | chr5: 79070077 | CMYA5.GENEBODY | R | | |
| cg09888186 | chr1: 183782195 | RGL1.GENEBODY | R | | |
| cg10294240 | chr14: 96686415 | BDKRB2.UTR5 | R | | |
| cg12194594 | chr13: 113426250 | ATP11A.GENEBODY | R | chr13: 113424898- | S_Shore |
| | | | | 113425105 | |
| cg13432241 | chr4: 154389285 | KIAA0922.GENEBODY | F | chr4: 154386834- | S_Shore |
| | | | | 154388183 | |
| cg17890764 | chr3: 52864816 | ITIH4.TSS200 | R | | |
| cg18491744 | chr8: 41580834 | ANK1.GENEBODY | F | chr8: 41583135-41583524 | N_Shelf |
| cg20069765 | chr6: 33036464 | HLA-DPA1.GENEBODY | R | | |
| cg21670451 | chr5: 139781334 | ANKHD1.TSS200 | F | chr5: 139780307- | Island |
| | | | | 139782108 | |
| cg23272088 | chr11: 2504813 | KCNQ1.GENEBODY | R | | |
| cg25227866 | chr17: 71489530 | SDK2.GENEBODY | R | | |
| cg00093829 | chr10: 6247101 | PFKFB3.GENEBODY | F | chr10: 6242975-6246457 | S_Shore |
| cg01027750 | chr19: 42636639 | POU2F2.TSS200 | F | chr19: 42637277-42637684 | N_Shore |
| cg02782292 | chr4: 144433846 | SMARCA5.TSS1500 | F | chr4: 144434249- | N_Shore |
| | | | | 144435343 | |
| cg03040622 | chr7: 5536937 | MIR589.TSS1500 | F | chr7: 5539957-5541567 | N_Shelf |
| cg03101058 | chr17: 71258936 | CPSF4L.TSS1500 | R | | |
| ce03499581 | chr15: 78384868 | SH2D7.TSS200 | R | | |
| cg04398759 | chr1: 203101066 | ADORA1.GENEBODY | R | chr1: 203097233- | S_Shelf |
| | | | | 203097496 | |
| cg04502517 | chr20: 62564156 | DNAJC5.UTR3 | R | chr20: 62562545-62562862 | S_Shore |
| cg04908961 | chr13: 92003278 | MIR17HG.GENEBODY | F | chr13: 91999541-92001441 | S_Shore |
| cg05030172 | chr19: 2078510 | MOBKL2A.GENEBODY | F | chr19: 2078162-2078511 | Island |
| cg05137443 | chr6: 33094069 | HLA-DPB2.GENEBODY | F | | |
| cg05299486 | chr17: 40609380 | ATP6VOA1.TSS1500 | R | chr17: 40610746-40611032 | N_Shore |
| cg05451388 | chr3: 183817678 | HTR3E.TSS1500 | F | | |
| cg05887270 | chr1: 26129627 | SEPN1.GENEBODY | R | | |
| cg07097026 | chr6: 44257942 | TCTE1.UTR5 | F | chr6: 44253897-44254150 | S_Shelf |
| cg07151747 | chr2: 158977473 | UPP2.GENEBODY | F | | |
| cg07992044 | chr11: 68096049 | LRP5.GENEBODY | F | | |
| cg08402572 | chr2: 97510201 | ANKRD23.TSS1500 | F | | |
| cg10097316 | chr12: 7966784 | SLC2A14.UTR3 | R | | |
| cg11356398 | chr10: 133783182 | BNIP3.GENEBODY | F | | |
| cg11891522 | chr5: 61605247 | KIF2A.GENEBODY | F | chr5: 61601617-61602930 | S_Shelf |
| cg12131414 | chr17: 72352968 | BTBD17.GENEBODY | F | chr17: 72352827-72353798 | Island |
| cg12524428 | chr5: 146268595 | PPP2R2B.UTR5 | F | | |
| cg12674490 | chr6: 31587470 | BAT2.TSS1500 | R | chr6: 31587779-31589024 | N_Shore |
| cg12738981 | chr1: 155828357 | SYT11.TSS1500 | R | chr1: 155829109- | N_Shore |
| | | | | 155829431 | |
| cg13187009 | chr20: 55965497 | RBM38.TSS1500 | R | chr20: 55966212-55966964 | N_Shore |
| cg13277337 | chr2: 240265810 | HDAC4.GENEBODY | R | | |
| cg13594542 | chr9: 138850547 | UBAC1.GENEBODY | R | chr9: 138852639- | N_Shelf |
| | | | | 138853439 | |
| cg13925678 | chr19: 3522946 | FZR1.UTR5 | F | | |
| cg14095761 | chr9: 35713238 | TLN1.GENEBODY | F | | |
| cg14168316 | chr9: 127642782 | GOLGA1.UTR3 | R | | |
| cg14173476 | chr17: 19190476 | EPN2.UTR5 | F | | |
| cg15031661 | chr1: 240256603 | FMN2.EXON1 | F | chr1: 240254959- | Island |
| | | | | 240257063 | |
| cg15844109 | chrX: 78003205 | LPAR4.EXON1 | F | | |
| cg16054907 | chr22: 50706065 | MAPK11.GENEBODY | R | chr22: 50705406-50706553 | Island |
| cg16518986 | chr7: 157511420 | PTPRN2.GENEBODY | F | | |
| cg17297045 | chr12: 125581343 | AACS.GENEBODY | R | chr12: 125578348- | S_Shelf |
| | | | | 125578588 | |
| cg17547928 | chr4: 737930 | PCGF3.GENEBODY | F | chr4: 738424-741517 | N_Shore |
| cg17846127 | chr15: 89940505 | LOC254559.GENEBODY | R | chr15: 89942592-89943853 | N_Shelf |
| cg18183642 | chr1: 44497694 | SLC6A9.TSS1500 | F | chr1: 44495629-44497112 | S_Shore |
| cg18315680 | chr1: 154833117 | KCNN3.GENEBODY | R | | |
| cg18927185 | chr6: 33091634 | HLA-DPB2.GENEBODY | F | | |
| cg19062108 | chr12: 57630834 | NDUFA4L2.GENEBODY | R | chr12: 57631932-57634578 | N_Shore |
| cg19482842 | chr2: 16790314 | FAM49A.UTR5 | F | | |
| cg19710814 | chr6: 33091596 | HLA-DPB2.GENEBODY | R | | |
| cg19995654 | chr12: 66628777 | IRAK3.GENEBODY | R | | |
| cg20574732 | chr7: 158277801 | PTPRN2.GENEBODY | F | chr7: 158277603- | Island |
| | | | | 158278027 | |
| cg20643991 | chr5: 121690136 | SNCAIP.UTR5 | F | | |
| cg21542666 | chr1: 248005715 | OR11L1.TSS1500 | R | | |
| cg21570811 | chr4: 3362574 | RGS12.GENEBODY | F | chr4: 3365194-3365415 | N_Shelf |
| cg21751684 | chr15: 89920887 | LOC254559.TSS1500 | R | chr15: 89920793-89922768 | Island |
| cg21830221 | chr1: 42334284 | HIVEP3.UTR5 | F | | |
| cg22646210 | chr11: 72380726 | PDE2A.TSS1500 | F | | |
| cg23105863 | chr14: 101011318 | BEGAIN.GENEBODY | F | chr14: 101012840- | N_Shore |
| | | | | 101013117 | |
| cg23331433 | chr6: 33039606 | HLA-DPA1.GENEBODY | F | | |
| cg23502492 | chr17: 42394379 | RUNDC3A.GENEBODY | R | chr17: 42392323-42393079 | S_Shore |
| cg24940706 | chr19: 1648682 | TCF3.GENEBODY | F | chr19: 1646117-1646713 | S_Shore |
| cg25057035 | chr6: 33053889 | HLA-DPB1.GENEBODY | R | | |
| cg25516633 | chr8: 101315846 | RNF19A.TSS1500 | F | | |
| cg26013412 | chr1: 247094486 | AHCTF1.GENEBODY | R | chr1: 247094485- | Island |
| | | | | 247095667 | |
| cg26164712 | chr11: 118754565 | CXCR5.UTR5 | F | | |
| cg26283963 | chr7: 29330752 | CHN2.GENEBODY | F | | |
| cg26603629 | chr7: 2752217 | AMZ1.GENEBODY | F | | |
| cg27184903 | chr15: 29285727 | APBA2.UTR5 | F | | |
| cg27191457 | chr15: 69105133 | ANP32A.GENEBODY | F | | |
| cg27244524 | chr3: 38494523 | LOC100128640.GENEBODY | F | chr3: 38495088-38497151 | N_Shore |
| cg27661104 | chr2: 232645033 | PDE6D.GENEBODY | F | chr2: 232645313- | N_Shore |
| | | | | 232646505 | |
| cg02982614 | chr11: 89391479 | FOLH1B.TSS1500 | R | | |
| cg12348588 | chr17: 72352897 | BTBD17.GENEBODY | R | chr17: 72352827-72353798 | Island |
| cg13993358 | chr5: 176304275 | UNC5A.GENEBODY | R | chr5: 176304965- | N_Shore |
| | | | | 176305166 | |
| cg19567891 | chr15: 89921083 | LOC254559.TSS200 | F | chr15: 89920793-89922768 | Island |
| cg20371142 | chr20: 17295221 | PCSK2.GENEBODY | F | chr20: 17296125-17296531 | N_Shore |
| cg20745419 | chr1: 151696417 | C1orf230.GENEBODY | F | chr1: 151693991- | S_Shelf |
| | | | | 151694282 | |
| cg24953213 | chr5: 172140 | PLEKHG4B.GENEBODY | R | chr5: 169578-169798 | S_Shelf |
| cg00088251 | chr1: 1449754 | ATAD3A.UTR5 | R | chr1: 1446878-1448205 | S_Shore |
| cg00481644 | chr15: 89921158 | LOC254559.TSS200 | F | chr15: 89920793-89922768 | Island |
| cg00547530 | chr16: 3559687 | CLUAP1.GENEBODY | F | | |
| cg00863271 | chr17: 76798776 | USP36.GENEBODY | F | chr17: 76798154-76798461 | S_Shore |
| cg01011403 | chr4: 56500362 | NMU.GENEBODY | F | chr4: 56501912-56502523 | N_Shore |
| cg01476047 | chr10: 73974007 | ASCC1.UTR5 | F | chr10: 73975671-73976115 | N_Shore |
| cg01477971 | chr2: 103405951 | TMEM182.GENEBODY | R | | |
| cg01663696 | chr12: 7942817 | NANOG.GENEBODY | F | | |
| cg01833923 | chr17: 47286719 | GNGT2.EXON1 | R | | |
| cg02140508 | chr12: 4659038 | RAD51AP1.GENEBODY | F | | |
| cg02478836 | chr22: 47510037 | TBC1D22A.GENEBODY | F | chr22: 47513202-47513585 | N_Shelf |
| cg03366884 | chr17: 8844506 | PIK3R5.UTR5 | R | | |
| cg03368665 | chr11: 89819442 | UBTFL1.EXON1 | R | | |
| cg03450107 | chr1: 41980863 | HIVEP3.GENEBODY | R | chr1: 41981788-41982043 | N_Shore |
| cg04625873 | chr11: 118754530 | CXCR5.TSS200 | F | | |
| cg05244974 | chr17: 8798734 | PIK3R5.GENEBODY | R | | |
| cg05680240 | chr6: 31765195 | LSM2.UTR3 | R | chr6: 31763240-31763905 | S_Shore |
| cg05766510 | chr7: 157454062 | PTPRN2.GENEBODY | R | chr7: 157453774- | Island |
| | | | | 157454208 | |
| cg05845757 | chr1: 36039885 | TFAP2E.GENEBODY | F | chr1: 36038927-36040051 | Island |
| cg05913474 | chr8: 144815204 | FAM83H.UTR5 | R | chr8: 144815203- | Island |
| | | | | 144816518 | |
| cg05926586 | chr15: 74592786 | CCDC33.GENEBODY | R | | |
| cg06788504 | chr6: 32974517 | HLA-DOA.UTR3 | F | chr6: 32975684-32975926 | N_Shore |
| cg06860422 | chr16: 788079 | NARFL.GENEBODY | F | chr16: 787070-787327 | S_Shore |
| cg07159802 | chr1: 247921568 | OR1C1.EXON1 | F | | |
| cg07344019 | chr15: 69337685 | MIR548H4.GENEBODY | F | | |
| cg07470396 | chr19: 54697539 | TSEN34.UTR3 | F | chr19: 54693034-54695497 | S_Shelf |
| cg08192337 | chr12: 124982002 | NCOR2.UTR5 | F | | |
| cg08210506 | chr14: 81456824 | TSHR.GENEBODY | F | | |
| cg08797864 | chr3: 185531258 | IGF2BP2.GENEBODY | R | | |
| cg08822746 | chr22: 31321152 | C22orf27.GENEBODY | R | chr22: 31318228-31318489 | S_Shelf |
| cg08856378 | chr17: 560590 | VPS53.GENEBODY | R | | |
| cg09274040 | chr10: 134994455 | KNDC1.GENEBODY | R | chr10: 134996318- | N_Shore |
| | | | | 134996548 | |
| cg09506037 | chr2: 134180560 | NCKAP5.GENEBODY | F | | |
| cg09900440 | chr6: 33033312 | HLA-DPA1.UTR3 | R | | |
| cg09910067 | chr7: 157357758 | PTPRN2.GENEBODY | R | chr7: 157361512- | N_Shelf |
| | | | | 157361805 | |
| cg10572969 | chr11: 13690188 | FAR1.TSS200 | F | chr11: 13689588-13690724 | Island |
| cg11156873 | chr5: 1491579 | LPCAT1.GENEBODY | R | chr5: 1489799-1490071 | S_Shore |
| cg11514462 | chr11: 121001293 | TECTA.GENEBODY | F | chr11: 120998386- | S_Shelf |
| | | | | 120998886 | |
| cg12192566 | chr5: 140011377 | CD14.UTR3 | F | chr5: 140011482- | N_Shore |
| | | | | 140012739 | |
| cg13337413 | chr17: 10728324 | PIRT.UTR3 | F | | |
| cg13607138 | chr20: 33539306 | GSS.GENEBODY | R | chr20: 33543082-33543846 | N_Shelf |
| cg14150071 | chr1: 33545982 | ADC.TSS1500 | F | chr1: 33546524-33547243 | N_Shore |
| cg14286660 | chr7: 1050071 | C7orf50.GENEBODY | F | chr7: 1048921-1049124 | S_Shore |
| cg14826031 | chr11: 75376361 | MAP6.GENEBODY | F | chr11: 75378416-75379882 | N_Shelf |
| cg15005255 | chr19: 18368728 | KIAA1683.GENEBODY | F | chr19: 18368482-18368911 | Island |
| cg15344504 | chr3: 31677078 | STT3B.GENEBODY | F | | |
| cg17045790 | chr12: 6128269 | VWF.GENEBODY | F | | |
| cg17771737 | chr19: 20150505 | ZNF682.TSS1500 | R | chr19: 20149855-20150175 | S_Shore |
| cg18554789 | chr7: 139432178 | HIPK2.GENEBODY | F | | |
| cg18559249 | chr17: 37760173 | NEUROD2.UTR3 | F | chr17: 37761691-37763551 | N_Shore |
| cg19402335 | chr1: 159410002 | OR10J1.EXON1 | R | | |
| cg20484653 | chr1: 204955033 | NFASC.GENEBODY | F | chr1: 204950982- | S_Shelf |
| | | | | 204951210 | |
| cg21045306 | chr12: 57637925 | STAC3.GENEBODY | R | chr12: 57636557-57637673 | S_Shore |
| cg21378690 | chrX: 71694534 | HDAC8.UTR3 | F | | |
| cg21745287 | chr12: 123464511 | ARL6IP4.TSS1500 | R | chr12: 123464820- | N_Shore |
| | | | | 123465972 | |
| cg22931292 | chr11: 17765568 | KCNC1.GENEBODY | R | | |
| cg24627621 | chr6: 42109176 | C6orf132.GENEBODY | R | chr6: 42109904-42110361 | N_Shore |
| cg24931632 | chr1: 47899667 | MGC12982.GENEBODY | F | chr1: 47899661-47900385 | Island |
| cg25199552 | chr1: 23350054 | KDM1A.GENEBODY | R | chr1: 23345814-23346677 | S_Shelf |
| cg25647583 | chr15: 91427184 | FES.TSS1500 | F | chr15: 91427540-91427905 | N_Shore |
| cg25716623 | chr15: 89921845 | LOC254559.GENEBODY | F | chr15: 89920793-89922768 | Island |
| cg26009944 | chr1: 52210504 | OSBPL9.UTR5 | F | | |
| cg26355228 | chr2: 3260869 | TSSC1.GENEBODY | F | | |
| cg26406292 | chr7: 75292248 | HIP1.GENEBODY | F | | |
| cg27113326 | chr7: 157454068 | PTPRN2.GENEBODY | R | chr7: 157453774- | Island |
| | | | | 157454208 | |
| cg00108992 | chr16: 12192325 | SNX29.GENEBODY | R | | |
| cg00168967 | chr8: 62505560 | ASPH.GENEBODY | R | | |
| cg00256675 | chr4: 802962 | CPLX1.GENEBODY | R | chr4: 798538-798984 | S_Shelf |
| cg00572387 | chr20: 1472914 | SIRPB2.TSS1500 | R | | |
| cg00582579 | chr8: 135533786 | ZFAT.GENEBODY | R | | |
| cg00629585 | chr9: 135853992 | GFI1B.TSS200 | R | | |
| cg00869989 | chr19: 2774197 | SGTA.UTR5 | F | chr19: 2774196-2774550 | Island |
| cg00957480 | chr17: 10728755 | PIRT.GENEBODY | R | | |
| cg01120761 | chr12: 7903170 | CLEC4C.TSS1500 | R | | |
| cg01953456 | chr3: 183817976 | HTR3E.UTR5 | R | | |
| cg02264238 | chr3: 51426377 | MANF.GENEBODY | R | chr3: 51428412-51429625 | N_Shelf |
| cg02308914 | chr3: 129290549 | PLXND1.GENEBODY | R | | |
| cg02590486 | chr12: 499376 | KDM5A.TSS1500 | R | chr12: 498098-498920 | S_Shore |
| cg02882755 | chr6: 34100963 | GRM4.EXON1 | R | | |
| cg02915544 | chr11: 89392829 | FOLH1B.UTR5 | R | | |
| cg03231860 | chr11: 128893940 | RICS.EXON1 | F | | |
| cg03243669 | chr7: 1931776 | MAD1L1.GENEBODY | F | | |
| cg03465847 | chrX: 40594937 | MED14.TSS200 | F | chrX: 40594515-40595457 | Island |
| cg03766174 | chr16: 81547907 | CMIP.GENEBODY | F | | |
| cg04428805 | chr17: 65239522 | HELZ.UTR5 | R | chr17: 65240725-65242498 | N_Shore |
| cg04756594 | chr16: 24857601 | SLC5A11.EXON1 | R | | |
| cg05647891 | chr4: 153346037 | FBXW7.UTR5 | F | | |
| cg05794927 | chr1: 248059252 | OR2W3.EXON1 | F | | |
| cg05938958 | chr1: 248224887 | OR2L3.EXON1 | F | | |
| cg06222979 | chr18: 14543469 | POTEC.UTR5 | F | | |
| cg06531741 | chr11: 113775450 | HTR3B.TSS200 | F | | |
| cg06969933 | chr16: 88099740 | BANP.GENEBODY | F | chr16: 88101473-88101911 | N_Shore |
| cg07474672 | chr6: 90142171 | ANKRD6.TSS1500 | F | chr6: 90142751-90143635 | N_Shore |
| cg07825294 | chr19: 1415757 | DAZAP1.GENEBODY | R | chr19: 1419751-1420405 | N_Shelf |
| cg07915189 | chr19: 7688621 | XAB2.GENEBODY | R | chr19: 7688620-7688832 | Island |
| cg08468599 | chr1: 2182126 | SKI.GENEBODY | F | | |
| cg08813944 | chr17: 71258589 | CPSF4L.TSS1500 | F | | |
| cg08917933 | chr1: 19615303 | AKR7A3.TSS200 | R | chr1: 19614998-19615660 | Island |
| cg08927102 | chr1: 1050110 | C1orf159.UTR5 | F | chr1: 1050515-1050830 | N_Shore |
| cg09419176 | chr7: 157670705 | PTPRN2.GENEBODY | R | chr7: 157669879- | S_Shore |
| | | | | 157670095 | |
| cg09445097 | chr9: 130186736 | ZNF79.UTR5 | R | chr9: 130186276- | Island |
| | | | | 130187160 | |
| cg09867656 | chr1: 76550508 | ST6GALNAC3.GENEBODY | R | | |
| cg09934892 | chr16: 3559551 | CLUAP1.GENEBODY | F | | |
| cg10100117 | chr1: 179843111 | TOR1AIP2.UTR5 | R | chr1: 179846879- | N_Shelf |
| | | | | 179847106 | |
| cg10329874 | chr11: 66621442 | PC.GENEBODY | R | chr11: 66623620-66626614 | N_Shelf |
| cg10409230 | chr17: 40318360 | KCNH4.GENEBODY | F | | |
| cg10487770 | chr5: 57879443 | RAB3C.GENEBODY | R | chr5: 57878725-57879177 | S_Shore |
| cg10719970 | chr6: 41529932 | FOXP4.UTR5 | R | chr6: 41528266-41528900 | S_Shore |
| cg11375616 | chr21: 47287017 | PCBP3.GENEBODY | R | chr21: 47287012-47287426 | Island |
| cg11397606 | chr1: 67151389 | SGIP1.GENEBODY | R | | |
| cg11661493 | chr17: 74423978 | UBE20.GENEBODY | F | | |
| cg11866943 | chr3: 52529351 | STAB1.TSS200 | F | | |
| cg12426652 | chr21: 37915056 | CLDN14.TSS200 | F | | |
| cg12991341 | chr8: 109800287 | TMEM74.TSS1500 | F | chr8: 109799429- | S_Shore |
| | | | | 109799847 | |
| cg13372513 | chr7: 158177056 | PTPRN2.GENEBODY | F | | |
| cg13534791 | chr9: 35042344 | C9orf131.EXON1 | F | | |
| cg13995599 | chr13: 113426270 | ATP11A.GENEBODY | R | chr13: 113424898- | S_Shore |
| | | | | 113425105 | |
| cg14274019 | chr9: 134184504 | PPAPDC3.UTR3 | R | chr9: 134183330- | S_Shore |
| | | | | 134183613 | |
| cg14879948 | chr5: 1394576 | SLC6A3.UTR3 | F | | |
| cg15147833 | chr1: 247921672 | OR1C1.EXON1 | R | | |
| cg15617023 | chr13: 40113248 | LHFP.GENEBODY | R | | |
| cg15871371 | chr3: 71274624 | FOXP1.UTR5 | F | | |
| cg15871766 | chr12: 52365222 | ACVR1B.GENEBODY | F | | |
| cg16748694 | chr10: 126754386 | CTBP2.UTR5 | R | | |
| cg16895672 | chr10: 34470745 | PARD3.GENEBODY | R | | |
| cg16983282 | chr17: 9871831 | GAS7.GENEBODY | R | | |
| cg17013166 | chr22: 30961151 | GAL3ST1.TSS1500 | R | | |
| cg17135722 | chr20: 17295815 | PCSK2.GENEBODY | R | chr20: 17296125-17296531 | N_Shore |
| cg17179826 | chr1: 204946032 | NFASC.UTR3 | R | | |
| cg17707347 | chr1: 234614967 | TARBP1.TSS200 | F | chr1: 234613936- | Island |
| | | | | 234614972 | |
| cg17810765 | chr2: 242127836 | ANO7.TSS200 | R | | |
| cg17912283 | chr17: 72350487 | KIF19.GENEBODY | F | chr17: 72350346-72350711 | Island |
| cg18502477 | chr11: 57946199 | OR9Q1.UTR5 | F | | |
| cg18812946 | chrX: 153140985 | L1CAM.GENEBODY | F | | |
| cg18861762 | chr2: 242127852 | ANO7.TSS200 | F | | |
| cg19148404 | chr3: 14443444 | SLC6A6.TSS1500 | F | chr3: 14443629-14444577 | N_Shore |
| cg19273683 | chr1: 21656047 | ECE1.GENEBODY | R | | |
| cg19325793 | chr6: 33082165 | HLA-DPB2.GENEBODY | F | chr6: 33084740-33084995 | N_Shelf |
| cg19393755 | chr17: 71258101 | CPSF4L.TSS200 | F | | |
| cg19405329 | chr15: 49440632 | COPS2.GENEBODY | F | | |
| cg19547192 | chr7: 158059976 | PTPRN2.GENEBODY | R | chr7: 158060894- | N_Shore |
| | | | | 158061100 | |
| cg19606462 | chr5: 7827964 | ADCY2.UTR3 | R | | |
| cg20682981 | chr17: 1962627 | HIC1.UTR3 | F | chr17: 1952919-1962328 | S_Shore |
| cg21029612 | chr15: 89921258 | LOC254559.TSS200 | R | chr15: 89920793-89922768 | Island |
| cg21552242 | chr3: 157814326 | SHOX2.UTR3 | R | chr3: 157815581- | N_Shore |
| | | | | 157816095 | |
| cg21703606 | chr12: 54427884 | HOXC4.UTR5 | R | chr12: 54427025-54428709 | Island |
| cg21900589 | chr8: 22866294 | RHOBTB2.GENEBODY | F | chr8: 22864324-22864588 | S_Shore |
| cg22791545 | chr17: 1924319 | RTN4RL1.GENEBODY | R | chr17: 1927051-1929408 | N_Shelf |
| cg22821554 | chr7: 99383155 | CYP3A4.TSS1500 | F | | |
| cg23055499 | chr12: 51847363 | SLC4A8.GENEBODY | F | | |
| cg23172995 | chr5: 150486801 | ANXA6.GENEBODY | R | | |
| cg23714707 | chr17: 17418691 | PEMT.GENEBODY | R | | |
| cg23833461 | chr17: 9929528 | GAS7.EXON1 | R | | |
| cg24603972 | chr10: 61434211 | SLC16A9.GENEBODY | F | | |
| cg25129480 | chr1: 185126704 | C1orf25.TSS1500 | F | chr1: 185125850- | Island |
| | | | | 185126705 | |
| cg25312122 | chr1: 160184732 | PEA15.UTR3 | R | | |
| cg25425390 | chr12: 42763958 | PPHLN1.GENEBODY | R | | |
| cg25985583 | chr7: 26404716 | SNX10.GENEBODY | R | | |
| cg26306056 | chr6: 32972989 | HLA-DOA.UTR3 | R | chr6: 32975684-32975926 | N_Shelf |
| cg26353877 | chr1: 159557886 | APCS.GENEBODY | R | | |
| cg26558420 | chr20: 30331315 | TPX2.UTR5 | R | chr20: 30326913-30327430 | S_Shelf |
| cg26577231 | chr19: 18368873 | KIAA1683.GENEBODY | F | chr19: 18368482-18368911 | Island |
| cg26942930 | chr7: 147804524 | CNTNAP2.GENEBODY | R | | |
| cg27150178 | chr19: 1456337 | APC2.GENEBODY | F | chr19: 1456077-1456347 | Island |
| cg01173794 | chr19: 36131225 | ETV2.TSS1500 | F | chr19: 36134153-36135781 | N_Shelf |
| cg24774182 | chr15: 22017473 | CXADRP2.TSS1500 | F | | |
| cg27541374 | chr17: 58042493 | RNFT1.TSS1500 | R | chr17: 58041846-58042463 | S_Shore |
| cg00401265 | chrX: 78003113 | LPAR4.TSS200 | F | | |
| cg00435063 | chr7: 157454207 | PTPRN2.GENEBODY | F | chr7: 157453774- | Island |
| | | | | 157454208 | |
| cg00515756 | chr11: 61464876 | DAGLA.UTR5 | R | | |
| cg00653387 | chr7: 137028535 | PTN.UTR5 | F | | |
| cg00808053 | chr12: 6125746 | VWF.GENEBODY | F | | |
| cg00808511 | chr16: 52495237 | TOX3.GENEBODY | R | | |
| cg01132064 | chr11: 46382562 | DGKZ.GENEBODY | F | | |
| cg01403030 | chr9: 19026479 | FAM154A.GENEBODY | R | chr9: 19027850-19028064 | N_Shore |
| cg01507200 | chr1: 247877066 | OR6F1.TSS1500 | R | | |
| cg01708636 | chr4: 166301085 | CPE.GENEBODY | R | chr4: 166299975- | S_Shore |
| | | | | 166300668 | |
| cg02077068 | chr16: 19872240 | GPRC5B.GENEBODY | R | | |
| cg03081691 | chr1: 226188006 | C1orf55.TSS1500 | F | chr1: 226186803- | S_Shore |
| | | | | 226187336 | |
| cg03306486 | chr19: 1467952 | APC2.GENEBODY | F | chr19: 1465206-1471241 | Island |
| cg04383012 | chr2: 231921692 | PSMD1.EXON1 | R | chr2: 231921413- | Island |
| | | | | 231921972 | |
| cg04655481 | chr9: 125796809 | GPR21.TSS200 | F | | |
| cg04665588 | chr17: 14243776 | HS3ST3B1.GENEBODY | F | | |
| cg04804648 | chr17: 72348295 | KIF19.GENEBODY | F | chr17: 72347924-72348322 | Island |
| cg04809274 | chr6: 32977254 | HLA-DOA.EXON1 | F | chr6: 32975684-32975926 | S_Shore |
| cg04962134 | chr11: 55653310 | SPRYD5.GENEBODY | R | | |
| cg05221455 | chr11: 829616 | EFCAB4A.GENEBODY | F | chr11: 829284-833558 | Island |
| cg05720937 | chr2: 98280362 | ACTR1B.EXON1 | R | chr2: 98280063-98280871 | Island |
| cg06517181 | chr10: 134994252 | KNDC1.GENEBODY | F | chr10: 134993121- | S_Shore |
| | | | | 134993324 | |
| cg06572563 | chr19: 1132966 | SBNO2.GENEBODY | R | | |
| cg07100542 | chr1: 17402079 | PADI2.GENEBODY | R | | |
| cg07436694 | chr16: 21638444 | METTL9.GENEBODY | R | | |
| cg07586799 | chr3: 195591722 | TNK2.GENEBODY | R | chr3: 195594836- | N_Shelf |
| | | | | 195595494 | |
| cg07870237 | chr5: 131348431 | ACSL6.TSS1500 | F | chr5: 131346892- | S_Shore |
| | | | | 131347776 | |
| cg08984900 | chr17: 34430281 | CCL4.TSS1500 | R | | |
| cg09524686 | chr11: 829182 | EFCAB4A.GENEBODY | F | chr11: 829284-833558 | N_Shore |
| cg09565226 | chr16: 84759648 | USP10.GENEBODY | R | | |
| cg10256976 | chr11: 20078682 | NAV2.GENEBODY | F | | |
| cg10327976 | chr15: 42843303 | HAUS2.GENEBODY | R | chr15: 42840462-42841116 | S_Shelf |
| cg10341513 | chr22: 51041242 | MAPK8IP2.TSS1500 | R | chr22: 51042293-51043422 | N_Shore |
| cg10521450 | chr10: 105439280 | SH3PXD2A.GENEBODY | R | | |
| cg10959048 | chr3: 93694020 | PROS1.TSS1500 | R | chr3: 93692554-93692783 | S_Shore |
| cg11049863 | chr17: 80832470 | TBCD.GENEBODY | F | chr17: 80829533-80829871 | S_Shelf |
| cg11150074 | chr1: 200863559 | C1orf106.TSS1500 | R | chr1: 200860076- | S_Shelf |
| | | | | 200860576 | |
| cg11321144 | chr4: 151178020 | DCLK2.UTR3 | F | chr4: 151177347- | S_Shore |
| | | | | 151177559 | |
| cg11521965 | chr7: 137028604 | PTN.TSS200 | F | | |
| cg11581706 | chr7: 82072141 | CACNA2D1.GENEBODY | F | chr7: 82072021-82073520 | Island |
| cg12196847 | chr14: 81426577 | TSHR.GENEBODY | R | | |
| cg12617146 | chr10: 97101836 | SORBS1.GENEBODY | R | | |
| cg13617795 | chr7: 105137683 | PUS7.GENEBODY | F | | |
| cg13698125 | chr9: 140391480 | PNPLA7.GENEBODY | R | chr9: 140389453- | S_Shore |
| | | | | 140389751 | |
| cg13921245 | chr6: 33053791 | HLA-DPB1.GENEBODY | F | | |
| cg14022090 | chr11: 34642303 | EHF.TSS1500 | F | | |
| cg14147151 | chr9: 138948000 | NACC2.UTR5 | R | chr9: 138941470- | S_Shelf |
| | | | | 138944021 | |
| cg14322298 | chr6: 10585683 | GCNT2.GENEBODY | F | | |
| cg14402562 | chr11: 8945810 | C11orf16.GENEBODY | R | | |
| cg15287806 | chr10: 237794 | ZMYND11.GENEBODY | R | | |
| cg15332132 | chr20: 62183964 | C20orf195.TSS1500 | R | chr20: 62185073-62185572 | N_Shore |
| cg15367000 | chr2: 239017149 | ESPNL.GENEBODY | F | | |
| cg15463309 | chr8: 141598157 | EIF2C2.GENEBODY | F | | |
| cg16182685 | chr2: 3260980 | TSSC1.GENEBODY | F | | |
| cg16280667 | chr11: 118754593 | CXCR5.UTR5 | F | | |
| cg16477417 | chr17: 73342168 | GRB2.GENEBODY | F | | |
| cg16711332 | chr1: 6531727 | PLEKHG5.GENEBODY | F | chr1: 6530536-6531814 | Island |
| cg16791781 | chr11: 986922 | AP2A2.GENEBODY | R | | |
| cg17777731 | chr6: 32976131 | HLA-DOA.GENEBODY | R | chr6: 32975684-32975926 | S_Shore |
| cg17947172 | chr1: 204966197 | NFASC.GENEBODY | R | | |
| cg18702239 | chrX: 69663606 | DLG3.TSS1500 | F | chrX: 69665102-69665355 | N_Shore |
| cg18715243 | chr8: 37658755 | GPR124.GENEBODY | R | chr8: 37654048-37656100 | S_Shelf |
| cg18969798 | chr4: 151157003 | DCLK2.GENEBODY | R | | |
| cg19185706 | chr10: 88475771 | LDB3.GENEBODY | F | chr10: 88470905-88471934 | S_Shelf |
| cg19409156 | chr15: 48413040 | SLC24A5.TSS200 | R | | |
| cg19427746 | chr17: 4889932 | CAMTA2.UTR5 | R | chr17: 4890282-4890991 | N_Shore |
| cg20100408 | chr6: 33050888 | HLA-DPB1.GENEBODY | F | chr6: 33048416-33048814 | S_Shelf |
| cg20541233 | chr21: 18938039 | CXADR.UTR3 | R | | |
| cg20907136 | chr6: 33041221 | HLA-DPA1.GENEBODY | F | | |
| cg20938170 | chr11: 75428910 | MOGAT2.TSS200 | R | | |
| cg21748244 | chr5: 169713179 | LCP2.GENEBODY | F | | |
| cg21776674 | chr1: 6160143 | KCNAB2.UTR3 | F | | |
| cg22007216 | chr4: 155312410 | DCHS2.GENEBODY | R | | |
| cg22381352 | chr1: 146742008 | CHD1L.GENEBODY | R | | |
| cg22442557 | chr17: 79011352 | BAIAP2.GENEBODY | R | chr17: 79008929-79009762 | S_Shore |
| cg22858170 | chr5: 39204386 | FYB.UTR5 | R | | |
| cg23095615 | chr6: 146136563 | FBXO30.TSS1500 | F | chr6: 146136325- | Island |
| | | | | 146136564 | |
| cg23326197 | chr7: 99382370 | CYP3A4.TSS1500 | F | | |
| cg23645261 | chr19: 30940563 | ZNF536.GENEBODY | F | chr19: 30941853-30942181 | N_Shore |
| cg23676767 | chr1: 226918736 | ITPKB.GENEBODY | R | | |
| cg23699947 | chr12: 27167077 | TM7SF3.EXON1 | F | chr12: 27166996-27167464 | Island |
| cg23777946 | chr18: 24125409 | KCTD1.UTR5 | R | chr18: 24126780-24131138 | N_Shore |
| cg23998119 | chr14: 77600217 | ZDHHC22.GENEBODY | R | | |
| cg24214000 | chr22: 50282386 | ZBED4.UTR3 | F | chr22: 50279568-50280376 | S_Shelf |
| cg24396358 | chr6: 32977400 | HLA-DOA.TSS200 | F | chr6: 32975684-32975926 | S_Shore |
| cg24436022 | chr22: 45691661 | UPK3A.UTR3 | F | | |
| cg24625894 | chr8: 145638202 | SLC39A4.GENEBODY | R | chr8: 145637965- | Island |
| | | | | 145639182 | |
| cg24678581 | chr5: 170882542 | FGF18.GENEBODY | R | chr5: 170883600- | N_Shore |
| | | | | 170883836 | |
| cg24938100 | chr6: 33091407 | HLA-DPB2.GENEBODY | R | | |
| cg25693265 | chr7: 6517860 | KDELR2.GENEBODY | R | | |
| cg26345142 | chr12: 109305794 | SVOP.UTR3 | F | | |
| cg26490671 | chr7: 157670732 | PTPRN2.GENEBODY | R | chr7: 157669879- | S_Shore |
| | | | | 157670095 | |
| cg26692016 | chr12: 7817844 | APOBEC1.GENEBODY | R | | |
| cg26871279 | chr11: 2423559 | TSSC4.UTR5 | R | chr11: 2421040-2422474 | S_Shore |
| cg27504782 | chr7: 94284258 | SGCE.GENEBODY | R | chr7: 94284858-94286527 | N_Shore |
| cg00731886 | chr4: 1363008 | KIAA1530.GENEBODY | F | chr4: 1364146-1364349 | N_Shore |
| cg00896220 | chr17: 34639327 | CCL4L1.TSS1500 | R | | |
| cg01443488 | chr1: 159557514 | APCS.TSS200 | F | | |
| cg01924561 | chr1: 43416103 | SLC2A1.GENEBODY | F | | |
| cg04810997 | chr7: 143140674 | TAS2R60.EXON1 | R | | |
| cg05677694 | chr6: 33052797 | HLA-DPB1.GENEBODY | F | chr6: 33048416-33048814 | S_Shelf |
| cg11070176 | chr11: 70489806 | SHANK2.GENEBODY | F | | |
| cg11941526 | chrX: 38660299 | MID1IP1.TSS1500 | F | chrX: 38662906-38664764 | N_Shelf |
| cg14517144 | chr10: 70098420 | HNRNPH3.GENEBODY | F | | |
| cg14684709 | chr13: 100547249 | CLYBL.UTR3 | F | chr13: 100547172- | Island |
| | | | | 100547431 | |
| cg16537306 | chr17: 9850268 | GAS7.GENEBODY | R | | |
| cg17753661 | chr1: 26441637 | PDIK1L.GENEBODY | F | chr1: 26437319-26438917 | S_Shelf |
| cg23710594 | chr1: 33841330 | PHC2.TSS200 | R | | |
| cg00935894 | chr8: 110705093 | GOLSYN.TSS1500 | R | chr8: 110703409- | S_Shore |
| | | | | 110703897 | |
| cg01083652 | chr15: 71629208 | THSD4.GENEBODY | R | | |
| cg02075590 | chr4: 125607552 | ANKRD50.UTR5 | R | | |
| cg02169973 | chr19: 50983986 | C19orf63.GENEBODY | F | chr19: 50983853-50984075 | Island |
| cg02220617 | chr14: 102312533 | PPP2R5C.GENEBODY | R | | |
| cg02555215 | chr12: 113497560 | DTX1.GENEBODY | R | chr12: 113494389- | S_Shelf |
| | | | | 113495534 | |
| cg02963493 | chr19: 11568915 | ELAVL3.GENEBODY | R | chr19: 11565384-11565711 | S_Shelf |
| cg03252568 | chr17: 41727259 | MEOX1.GENEBODY | F | chr17: 41723219-41723826 | S_Shelf |

TABLE 5
Top 1000 methylation sites associated with the SCLC-N subtype

| Methylation Site | Position (GRCh 37) | Gene | Strand | CpG Island Name | Relation to CpG Island |
|---|---|---|---|---|---|
| cg23317857 | chr6: 41696297 | TFEB.UTR5 | F | | |
| cg16860004 | chr8: 103137229 | NCALD.TSS1500 | F | chr8: 103135914-103136775 | S_Shore |
| cg20149780 | chr1: 15751063 | EFHD2.GENEBODY | F | | |
| cg00448761 | chr19: 6066102 | RFX2.UTR5 | R | | |
| cg01942646 | chr1: 27240694 | NROB2.TSS200 | R | | |
| cg19343518 | chr6: 157508679 | ARID1B.GENEBODY | F | | |
| cg03332546 | chr19: 16553887 | EPS15L1.GENEBODY | F | | |
| cg16177693 | chr1: 27240669 | NR0B2.TSS200 | R | | |
| cg23861617 | chr4: 83422170 | TMEM150C.GENEBODY | F | | |
| cg21147709 | chr1: 12004662 | PLOD1.GENEBODY | R | | |
| cg26713273 | chr12: 11906243 | ETV6.GENEBODY | R | | |
| cg01570265 | chr14: 70180751 | KIAA0247.UTR3 | R | | |
| cg02573468 | chr15: 41224537 | DLL4.GENEBODY | F | chr15: 41217789-41223180 | S_Shore |
| cg19280540 | chr19: 16553711 | EPS15L1.GENEBODY | R | | |
| cg05552035 | chr8: 37699239 | GPR124.GENEBODY | F | chr8: 37698846-37699931 | Island |
| cg22016731 | chr1: 40311142 | TRIT1.GENEBODY | F | | |
| cg15578140 | chr7: 147718109 | MIR548F3.GENEBODY | R | | |
| cg03561416 | chr6: 157254584 | ARID1B.GENEBODY | F | | |
| cg08482167 | chr6: 144066847 | PHACTR2.GENEBODY | R | | |
| cg25167574 | chr4: 79861398 | PAQR3.TSS1500 | F | chr4: 79860213-79860745 | S_Shore |
| cg23430388 | chr3: 63429828 | SYNPR.GENEBODY | F | | |
| cg21881338 | chr12: 125566189 | AACS.GENEBODY | F | chr12: 125569828-125570140 | N_Shelf |
| cg24500959 | chr10: 99474813 | MARVELD1.GENEBODY | R | chr10: 99473084-99473291 | S_Shore |
| cg07099407 | chr6: 43027930 | KLC4.TSS1500 | R | chr6: 43026939-43027841 | S_Shore |
| cg13464573 | chr2: 7172097 | RNF144A.GENEBODY | F | chr2: 7172296-7172512 | N_Shore |
| cg21436938 | chr9: 116061416 | RNF183.TSS200 | F | | |
| cg10173620 | chr15: 89658904 | ABHD2.UTR5 | R | | |
| cg09337391 | chr20: 45871363 | ZMYND8.GENEBODY | R | chr20: 45867654-45868111 | S_Shelf |
| cg19307500 | chr19: 1083193 | HMHA1.GENEBODY | R | chr19: 1082576-1083430 | Island |
| cg14651919 | chr17: 74075189 | ZACN.TSS200 | R | chr17: 74070404-74073530 | S_Shore |
| cg04645676 | chr5: 10655569 | ANKRD33B.UTR3 | F | | |
| cg20731121 | chr3: 111636305 | PHLDB2.GENEBODY | F | | |
| cg25727520 | chr2: 37576821 | QPCT.GENEBODY | R | | |
| cg26003056 | chr5: 89908195 | GPR98.GENEBODY | F | | |
| cg00072720 | chr17: 7165831 | CLDN7.TSS200 | R | chr17: 7164285-7166245 | Island |
| cg05564438 | chr11: 64120286 | CCDC88B.GENEBODY | R | | |
| cg21763058 | chr12: 49740976 | DNAJC22.TSS200 | R | chr12: 49738680-49740841 | S_Shore |
| cg00325866 | chr15: 62359130 | C2CD4A.TSS200 | F | chr15: 62359827-62360995 | N_Shore |
| cg04868764 | chr15: 41137019 | SPINT1.GENEBODY | F | chr15: 41135719-41137210 | Island |
| cg16609181 | chr19: 1083007 | HMHA1.GENEBODY | R | chr19: 1082576-1083430 | Island |
| cg05767421 | chr8: 120221185 | MAL2.GENEBODY | R | chr8: 120220388-120221222 | Island |
| cg12379775 | chr20: 62669791 | NCRNA00176.GENEBODY | F | chr20: 62669304-62670730 | Island |
| cg26215113 | chr2: 234369962 | DGKD.GENEBODY | R | chr2: 234369786-234370099 | Island |
| cg02707152 | chr11: 64980819 | SLC22A20.TSS1500 | R | | |
| cg25718402 | chr17: 7165909 | CLDN7.TSS200 | F | chr17: 7164285-7166245 | Island |
| cg11944076 | chr15: 51997390 | SCG3.GENEBODY | R | | |
| cg17519949 | chr19: 15292440 | NOTCH3.GENEBODY | R | chr19: 15292399-15292632 | Island |
| cg18777231 | chr20: 62669807 | NCRNA00176.GENEBODY | F | chr20: 62669304-62670730 | Island |
| cg27269561 | chr16: 3072713 | HCFC1R1.UTR3 | F | chr16: 3073686-3074443 | N_Shore |
| cg13275679 | chr17: 73151149 | HN1.TSS1500 | R | chr17: 73149830-73150885 | S_Shore |
| cg11785537 | chr12: 6484729 | SCNN1A.GENEBODY | F | | |
| cg27430662 | chr1: 109656708 | KIAA1324.UTR5 | R | chr1: 109656095-109656611 | S_Shore |
| cg01804664 | chr5: 149669414 | CAMK2A.TSS200 | F | | |
| cg22514229 | chr1: 94118054 | BCAR3.GENEBODY | R | | |
| cg09915444 | chr15: 62359275 | C2CD4A.EXON1 | F | chr15: 62359827-62360995 | N_Shore |
| cg20143111 | chr4: 10673188 | CLNK.UTR5 | R | | |
| cg12609243 | chr3: 54155240 | CACNA2D3.TSS1500 | R | chr3: 54155239-54157025 | Island |
| cg25191573 | chr3: 49037070 | P4HTM.GENEBODY | R | | |
| cg02910913 | chr17: 72968277 | C17orf28.GENEBODY | R | chr17: 72968167-72969164 | Island |
| cg21275690 | chr16: 3072970 | HCFC1R1.GENEBODY | F | chr16: 3073686-3074443 | N_Shore |
| cg03893692 | chr5: 149669484 | CAMK2A.TSS200 | F | | |
| cg11744304 | chr7: 100875822 | CLDN15.GENEBODY | R | chr7: 100875555-100876212 | Island |
| cg21486510 | chr2: 7171998 | RNF144A.GENEBODY | F | chr2: 7172296-7172512 | N_Shore |
| cg21761356 | chr12: 97892013 | RMST.GENEBODY | F | | |
| cg17553353 | chr3: 111718565 | TAGLN3.GENEBODY | R | | |
| cg02703870 | chr16: 82045140 | SDR42E1.TSS200 | R | chr16: 82044886-82045306 | Island |
| cg02641676 | chr8: 42397764 | C8orf40.UTR5 | R | chr8: 42396235-42397195 | S_Shore |
| cg06829681 | chr11: 12867453 | TEAD1.GENEBODY | F | | |
| cg09565670 | chr19: 39368408 | RINL.UTR5 | R | | |
| cg15873327 | chr19: 8398677 | KANK3.GENEBODY | F | chr19: 8397958-8400461 | Island |
| cg25960403 | chr4: 11370015 | MIR572.TSS1500 | F | chr4: 11370229-11370886 | N_Shore |
| cg01643250 | chr19: 15292485 | NOTCH3.GENEBODY | R | chr19: 15292399-15292632 | Island |
| cg11445764 | chr5: 147773164 | FBXO38.UTR5 | R | | |
| cg20147326 | chr11: 33913538 | LMO2.EXON1 | R | | |
| cg24994355 | chr10: 99445520 | AVPI1.UTR5 | F | chr10: 99446696-99447400 | N_Shore |
| cg10965131 | chr7: 151078995 | WDR86.GENEBODY | R | chr7: 151078462-151079536 | Island |
| cg17522866 | chr5: 147052374 | JAKMIP2.UTR5 | F | | |
| cg25147139 | chr12: 49740718 | DNAJC22.TSS1500 | R | chr12: 49738680-49740841 | Island |
| cg26317549 | chr15: 48497296 | SLC12A1.TSS1500 | R | | |
| cg02737268 | chr20: 3780182 | CDC25B.GENEBODY | F | chr20: 3776198-3776419 | S_Shelf |
| cg26808293 | chr16: 3072207 | TNFRSF12A.UTR3 | R | chr16: 3073686-3074443 | N_Shore |
| cg15298719 | chr17: 7165255 | CLDN7.EXON1 | R | chr17: 7164285-7166245 | Island |
| cg01609559 | chr6: 46138957 | ENPP5.TSS1500 | R | | |
| cg21445398 | chr2: 125425654 | CNTNAP5.GENEBODY | F | | |
| cg02659920 | chr11: 726335 | EPS8L2.GENEBODY | F | chr11: 725596-726870 | Island |
| cg03852018 | chr2: 149634410 | KIF5C.GENEBODY | F | chr2: 149632682-149633882 | S_Shore |
| cg04801402 | chr6: 46138912 | ENPP5.TSS200 | F | | |
| cg08472795 | chr2: 129067253 | HS6ST1.GENEBODY | F | | |
| cg08918013 | chr19: 55953951 | SHISA7.EXON1 | R | chr19: 55953371-55954641 | Island |
| cg25693302 | chr18: 55862678 | NEDD4L.UTR5 | R | chr18: 55862653-55862873 | Island |
| cg07701837 | chr2: 210516795 | MAP2.UTR5 | F | | |
| cg16371032 | chr8: 95654541 | ESRP1.GENEBODY | F | chr8: 95653898-95654733 | Island |
| cg10207240 | chr12: 122356781 | WDR66.UTR5 | R | chr12: 122356315-122356853 | Island |
| cg02017200 | chr19: 49256361 | FUT1.UTR5 | F | chr19: 49255778-49256495 | Island |
| cg03477240 | chr6: 20583779 | CDKAL1.GENEBODY | F | | |
| cg04687437 | chr19: 511113 | C19orf20.GENEBODY | F | chr19: 506526-507133 | S_Shelf |
| cg09195271 | chr1: 20142497 | RNF186.TSS1500 | R | | |
| cg10515731 | chr2: 220378996 | ACCN4.UTR5 | F | chr2: 220377743-220377946 | S_Shore |
| cg22776856 | chr16: 3072572 | THOC6.TSS1500 | R | chr16: 3073686-3074443 | N_Shore |
| cg25846061 | chr2: 236415673 | AGAP1.GENEBODY | R | | |
| cg01226742 | chr8: 37699923 | GPR124.UTR3 | F | chr8: 37698846-37699931 | Island |
| cg04304450 | chr22: 43525431 | BIK.UTR3 | F | | |
| cg04355435 | chr1: 43736290 | TMEM125.UTR5 | F | chr1: 43738484-43738829 | N_Shelf |
| cg08374799 | chr12: 53601050 | ITGB7.TSS200 | R | | |
| cg10483275 | chr5: 149669368 | CAMK2A.UTR5 | F | | |
| cg06961289 | chr1: 156722410 | HDGF.TSS200 | F | chr1: 156720747-156722069 | S_Shore |
| cg09726982 | chr2: 220379016 | ACCN4.UTR5 | F | chr2: 220377743-220377946 | S_Shore |
| cg17204557 | chr2: 121223909 | LOC84931.GENEBODY | R | | |
| cg19778003 | chr3: 111718062 | TAGLN3.EXON1 | R | | |
| cg26064870 | chr8: 102944342 | NCALD.UTR5 | R | | |
| cg13077545 | chr17: 41832873 | SOST.GENEBODY | R | chr17: 41832717-41833195 | Island |
| cg26549084 | chr3: 171759895 | FNDC3B.UTR5 | F | chr3: 171757493-171758724 | S_Shore |
| cg27469783 | chr19: 3801404 | MATK.UTR5 | F | chr19: 3801392-3801819 | Island |
| cg03310469 | chr2: 45236672 | SIX2.TSS200 | F | chr2: 45235511-45237792 | Island |
| cg21151061 | chr12: 50498016 | GPD1.GENEBODY | R | | |
| cg14034852 | chr17: 7165010 | CLDN7.GENEBODY | F | chr17: 7164285-7166245 | Island |
| cg17111837 | chr20: 62669757 | NCRNA00176.GENEBODY | R | chr20: 62669304-62670730 | Island |
| cg20126106 | chr3: 111718135 | TAGLN3.EXON1 | R | | |
| cg05304366 | chr15: 40226905 | EIF2AK4.GENEBODY | F | chr15: 40226210-40226631 | S_Shore |
| cg21191365 | chr14: 90529014 | KCNK13.GENEBODY | F | chr14: 90526696-90528951 | S_Shore |
| cg14186816 | chr18: 55862763 | NEDD4L.UTR5 | F | chr18: 55862653-55862873 | Island |
| cg18993457 | chr11: 441983 | ANO9.UTR5 | R | | |
| cg08442823 | chr11: 45951803 | PHF21A.UTR3 | F | | |
| cg13002109 | chr3: 124041146 | KALRN.GENEBODY | F | | |
| cg14914552 | chr8: 97340188 | PTDSS1.GENEBODY | R | | |
| cg18335326 | chr6: 30653659 | KIAA1949.GENEBODY | F | chr6: 30654392-30654934 | N_Shore |
| cg04671541 | chr3: 111717682 | TAGLN3.EXON1 | F | | |
| cg07251887 | chr17: 73641809 | LOC100130933.TSS1500 | R | | |
| cg00010853 | chr6: 30653167 | KIAA1949.GENEBODY | F | chr6: 30654392-30654934 | N_Shore |
| cg15245625 | chr18: 55862676 | NEDD4L.UTR5 | R | chr18: 55862653-55862873 | Island |
| cg24750887 | chr4: 89534723 | HERC3.GENEBODY | R | | |
| cg12473285 | chr17: 41832975 | SOST.GENEBODY | R | chr17: 41832717-41833195 | Island |
| cg08196842 | chr14: 65016517 | C14orf50.TSS200 | R | chr14: 65016516-65016909 | Island |
| cg08841552 | chr9: 140444378 | PNPLA7.GENEBODY | F | chr9: 140445653-140446891 | N_Shore |
| cg20465143 | chr8: 144653962 | C8orf73.GENEBODY | F | chr8: 144653774-144653976 | Island |
| cg21563471 | chr2: 158184535 | ERMN.TSS1500 | R | | |
| cg14770958 | chr3: 111717793 | TAGLN3.EXON1 | R | | |
| cg26068911 | chr5: 89967277 | GPR98.GENEBODY | F | | |
| cg27281690 | chr2: 7171963 | RNF144A.GENEBODY | F | chr2: 7172296-7172512 | N_Shore |
| cg10828127 | chr21: 41550814 | DSCAM.GENEBODY | R | chr21: 41554618-41554959 | N_Shelf |
| cg15554875 | chr2: 45236686 | SIX2.TSS200 | F | chr2: 45235511-45237792 | Island |
| cg05081953 | chr2: 158184543 | ERMN.TSS1500 | F | | |
| cg09717545 | chr19: 917707 | KISS1R.EXON1 | R | chr19: 917034-920787 | Island |
| cg11104313 | chr3: 111717534 | TAGLN3.TSS200 | F | | |
| cg11672757 | chr2: 241080172 | OTOS.TSS200 | R | | |
| cg15169077 | chr6: 46139019 | ENPP5.TSS1500 | F | | |
| cg04245402 | chr19: 751241 | C19orf21.UTR5 | R | | |
| cg08200404 | chr15: 62359539 | C2CD4A.UTR5 | F | chr15: 62359827-62360995 | N_Shore |
| cg10245608 | chr9: 92221282 | GADD45G.UTR3 | R | chr9: 92219771-92221411 | Island |
| cg21068094 | chr18: 55862711 | NEDD4L.UTR5 | R | chr18: 55862653-55862873 | Island |
| cg04994773 | chr1: 109656777 | KIAA1324.UTR5 | R | chr1: 109656095-109656611 | S_Shore |
| cg10976861 | chr2: 239149937 | HES6.TSS1500 | F | chr2: 239147592-239149900 | S_Shore |
| cg16638385 | chr17: 41832753 | SOST.GENEBODY | R | chr17: 41832717-41833195 | Island |
| cg22618164 | chr12: 122356400 | WDR66.TSS200 | F | chr12: 122356315-122356853 | Island |
| cg07028869 | chr12: 49741164 | DNAJC22.UTR5 | R | chr12: 49738680-49740841 | S_Shore |
| cg07354371 | chr1: 43736391 | TMEM125.UTR5 | R | chr1: 43738484-43738829 | N_Shelf |
| cg07637741 | chr6: 159084426 | SYTL3.GENEBODY | F | | |
| cg04886060 | chr16: 82044788 | SDR42E1.UTR5 | F | chr16: 82044886-82045306 | N_Shore |
| cg16931969 | chr4: 113985495 | ANK2.GENEBODY | F | | |
| cg24652919 | chr16: 3073191 | HCFC1R1.GENEBODY | R | chr16: 3073686-3074443 | N_Shore |
| cg06537333 | chr16: 85643863 | KIAA0182.TSS1500 | F | chr16: 85644089-85644323 | N_Shore |
| cg14052044 | chr19: 751159 | C19orf21.EXON1 | R | chr19: 746300-747161 | S_Shelf |
| cg18070562 | chr17: 8013617 | ALOXE3.GENEBODY | R | chr17: 8013172-8013657 | Island |
| cg25736482 | chr19: 54677232 | MBOAT7.UTR3 | R | | |
| cg27417294 | chr10: 81904244 | PLAC9.GENEBODY | R | | |
| cg27536453 | chr3: 169530417 | LRRC34.TSS200 | F | chr3: 169529875-169530435 | Island |
| cg03646740 | chr13: 114813015 | RASA3.GENEBODY | R | chr13: 114813942-114814457 | N_Shore |
| cg11482099 | chr11: 31833477 | PAX6.TSS1500 | F | chr11: 31831620-31839038 | Island |
| cg18998911 | chr3: 111718245 | TAGLN3.EXON1 | R | | |
| cg17749454 | chr17: 73101934 | SLC16A5.GENEBODY | R | chr17: 73105229-73106667 | N_Shelf |
| cg02449373 | chr19: 49256123 | FUT1.UTR5 | F | chr19: 49255778-49256495 | Island |
| cg03751944 | chr19: 1081936 | HMHA1.GENEBODY | F | chr19: 1079682-1081937 | Island |
| cg05251389 | chr22: 43525330 | BIK.UTR3 | F | | |
| cg15559684 | chr3: 63428234 | SYNPR.GENEBODY | R | | |
| cg27274706 | chr11: 129685751 | TMEM45B.EXON1 | R | chr11: 129685737-129686211 | Island |
| cg04930469 | chr17: 41832882 | SOST.GENEBODY | R | chr17: 41832717-41833195 | Island |
| cg06839900 | chr1: 2461900 | HES5.TSS1500 | R | chr1: 2460760-2462010 | Island |
| cg03018001 | chr19: 751098 | C19orf21.TSS200 | R | chr19: 746300-747161 | S_Shelf |
| cg08253704 | chr6: 30653242 | KIAA1949.GENEBODY | R | chr6: 30654392-30654934 | N_Shore |
| cg15925940 | chr14: 105330630 | KIAA0284.TSS1500 | R | chr14: 105330514-105332186 | Island |
| cg18612589 | chr17: 7165847 | CLDN7.TSS200 | R | chr17: 7164285-7166245 | Island |
| cg13401831 | chr22: 18593307 | TUBA8.TSS1500 | R | chr22: 18593113-18594197 | Island |
| cg20234170 | chr11: 34460107 | CAT.TSS1500 | F | chr11: 34460165-34460857 | N_Shore |
| cg22481263 | chr1: 906493 | PLEKHN1.GENEBODY | R | chr1: 906296-906538 | Island |
| cg03021329 | chr3: 124554058 | ITGB5.GENEBODY | F | | |
| cg07720856 | chr2: 232572668 | PTMA.TSS1500 | F | chr2: 232571137-232575833 | Island |
| cg15602548 | chr2: 54786148 | SPTBN1.GENEBODY | F | chr2: 54785026-54785969 | S_Shore |
| cg20925462 | chr3: 111717806 | TAGLN3.TSS200 | R | | |
| cg00510447 | chr16: 3071608 | TNFRSF12A.GENEBODY | F | chr16: 3070095-3071305 | S_Shore |
| cg07460919 | chr3: 183980626 | ECE2.GENEBODY | R | chr3: 183977548-183980633 | Island |
| cg14194715 | chr13: 32651552 | FRY.GENEBODY | F | | |
| cg05962028 | chr12: 214000 | IQSEC3.GENEBODY | F | chr12: 213763-214227 | Island |
| cg14245347 | chr6: 29856426 | HLA-H.GENEBODY | R | chr6: 29855295-29856565 | Island |
| cg17889402 | chr18: 55862872 | NEDD4L.UTR5 | F | chr18: 55862653-55862873 | Island |
| cg21521758 | chr7: 150036287 | RARRES2.GENEBODY | F | chr7: 150037459-150039031 | N_Shore |
| cg00282244 | chr7: 100876136 | CLDN15.GENEBODY | F | chr7: 100875555-100876212 | Island |
| cg01062519 | chr17: 34122169 | MMP28.GENEBODY | F | chr17: 34122024-34122801 | Island |
| cg13302994 | chr2: 210714233 | UNC80.GENEBODY | F | | |
| cg16341266 | chr7: 147256367 | MIR548I4.TSS1500 | F | | |
| cg17265693 | chr17: 7166490 | CLDN7.TSS1500 | F | chr17: 7164285-7166245 | S_Shore |
| cg17956387 | chr1: 208418015 | PLXNA2.TSS1500 | R | chr1: 208416489-208418016 | Island |
| cg12930882 | chr5: 37825585 | GDNF.GENEBODY | R | | |
| cg26335620 | chr19: 48215149 | EHD2.TSS1500 | F | chr19: 48216486-48216837 | N_Shore |
| cg11356571 | chr19: 48219924 | EHD2.GENEBODY | R | chr19: 48219831-48220298 | Island |
| cg03469805 | chr14: 105330648 | KIAA0284.TSS1500 | F | chr14: 105330514-105332186 | Island |
| cg04608356 | chr1: 27668433 | SYTL1.TSS200 | R | | |
| cg06644669 | chr6: 30653799 | KIAA1949.UTR5 | R | chr6: 30654392-30654934 | N_Shore |
| cg09865760 | chr12: 49739893 | DNAJC22.TSS1500 | R | chr12: 49738680-49740841 | Island |
| cg02512352 | chr7: 151137048 | CRYGN.EXON1 | F | chr7: 151136933-151137273 | Island |
| cg05098417 | chr11: 64067858 | C11orf20.TSS200 | F | chr11: 64066757-64068741 | Island |
| cg06740893 | chr1: 12251869 | TNFRSF1B.GENEBODY | R | chr1: 12251777-12251985 | Island |
| cg12934258 | chr6: 159084599 | SYTL3.GENEBODY | F | | |
| cg21604615 | chr1: 27668610 | SYTL1.EXON1 | F | | |
| cg01562385 | chr19: 10698098 | AP1M2.TSS200 | R | chr19: 10697786-10698099 | Island |
| cg17420360 | chr12: 49740840 | DNAJC22.TSS200 | F | chr12: 49738680-49740841 | Island |
| cg26020521 | chr11: 8680436 | TRIM66.TSS200 | F | | |
| cg26877508 | chr15: 71495324 | THSD4.GENEBODY | R | | |
| cg05305327 | chr20: 32156436 | CBFA2T2.GENEBODY | R | | |
| cg03738134 | chr8: 144653855 | C8orf73.GENEBODY | R | chr8: 144653774-144653976 | Island |
| cg17236870 | chr4: 94122883 | GRID2.GENEBODY | F | | |
| cg08439728 | chr6: 31097073 | PSORS1C1.UTR5 | F | | |
| cg01930441 | chr16: 21289268 | CRYM.GENEBODY | F | chr16: 21289419-21289819 | N_Shore |
| cg05639747 | chr12: 49740942 | DNAJC22.TSS200 | R | chr12: 49738680-49740841 | S_Shore |
| cg25989783 | chr8: 102699486 | NCALD.UTR3 | F | | |
| cg01864032 | chrX: 69674332 | DLG3.GENEBODY | F | chrX: 69674799-69675305 | N_Shore |
| cg12503394 | chr7: 1596118 | TMEM184A.TSS200 | R | | |
| cg16884042 | chr2: 223289683 | SGPP2.GENEBODY | F | chr2: 223288922-223290013 | Island |
| cg26785346 | chr4: 186732936 | SORBS2.UTR5 | R | | |
| cg01708552 | chr7: 100815485 | C7orf52.GENEBODY | R | chr7: 100815484-100816995 | Island |
| cg02487823 | chr3: 197440451 | KIAA0226.GENEBODY | F | | |
| cg13036352 | chr6: 30651825 | KIAA1949.GENEBODY | F | chr6: 30654392-30654934 | N_Shelf |
| cg13478289 | chr6: 33132650 | COL11A2.GENEBODY | F | chr6: 33129291-33129718 | S_Shelf |
| cg18010718 | chr17: 74075195 | ZACN.TSS200 | R | chr17: 74070404-74073530 | S_Shore |
| cg19095827 | chr3: 107243066 | BBX.UTR5 | F | chr3: 107241845-107242731 | S_Shore |
| cg23350812 | chr2: 236415734 | AGAP1.GENEBODY | R | | |
| cg01033299 | chr12: 53601015 | ITGB7.TSS200 | R | | |
| cg02492946 | chr1: 21667630 | ECE1.GENEBODY | R | chr1: 21671360-21672319 | N_Shelf |
| cg26575738 | chr17: 44896168 | WNT3.TSS200 | F | chr17: 44895800-44897025 | Island |
| cg00715124 | chr6: 3227026 | TUBB2B.GENEBODY | R | chr6: 3227025-3229688 | Island |
| cg03182958 | chr2: 223288923 | SGPP2.TSS1500 | R | chr2: 223288922-223290013 | Island |
| cg05650846 | chr16: 3078142 | CCDC64B.GENEBODY | R | chr16: 3078141-3078553 | Island |
| cg10544251 | chr1: 156215727 | PAQR6.UTR5 | R | chr1: 156215368-156215899 | Island |
| cg03230858 | chr19: 751124 | C19orf21.TSS200 | R | chr19: 746300-747161 | S_Shelf |
| cg03880611 | chr5: 31889373 | PDZD2.GENEBODY | F | | |
| cg21094405 | chr13: 113656117 | MCF2L.GENEBODY | R | chr13: 113656116-113656379 | Island |
| cg22807241 | chr7: 147709862 | MIR548F3.GENEBODY | R | | |
| cg24837219 | chr3: 132772692 | TMEM108.UTR5 | R | | |
| cg01100330 | chr22: 39639518 | PDGFB.GENEBODY | R | chr22: 39638219-39640966 | Island |
| cg19477361 | chr19: 2607903 | GNG7.UTR5 | R | chr19: 2607725-2607967 | Island |
| cg22453183 | chr5: 89940953 | GPR98.GENEBODY | F | | |
| cg09701233 | chr2: 236415814 | AGAP1.GENEBODY | R | | |
| cg18814808 | chr5: 143698198 | KCTD16.GENEBODY | R | | |
| cg19529471 | chr13: 35517634 | NBEA.GENEBODY | R | | |
| cg15766101 | chr19: 46519228 | CCDC61.GENEBODY | R | chr19: 46518283-46520080 | Island |
| cg17499826 | chr19: 46284986 | DMPK.GENEBODY | F | chr19: 46282544-46283127 | S_Shore |
| cg22540067 | chr2: 125305001 | CNTNAP5.GENEBODY | F | | |
| cg02445972 | chr17: 7166593 | CLDN7.TSS1500 | R | chr17: 7164285-7166245 | S_Shore |
| cg23098020 | chr1: 153651886 | NPR1.EXON1 | F | chr1: 153650830-153652375 | Island |
| cg24302354 | chr19: 48219029 | EHD2.UTR5 | R | chr19: 48219831-48220298 | N_Shore |
| cg19469357 | chr7: 8474830 | NXPH1.UTR5 | R | chr7: 8473139-8475199 | Island |
| cg00144550 | chr2: 100937796 | LONRF2.GENEBODY | R | chr2: 100937779-100939059 | Island |
| cg11303127 | chr12: 49740807 | DNAJC22.TSS1500 | F | chr12: 49738680-49740841 | Island |
| cg27320207 | chr19: 15307057 | NOTCH3.GENEBODY | R | chr19: 15306243-15307111 | Island |
| cg03042971 | chr17: 47301348 | PHOSPHO1.UTR3 | R | chr17: 47301545-47302477 | N_Shore |
| cg05020685 | chr4: 40858965 | APBB2.GENEBODY | F | chr4: 40858884-40859162 | Island |
| cg07035875 | chr16: 52557205 | TOX3.UTR5 | R | | |
| cg12105190 | chr6: 30653407 | KIAA1949.GENEBODY | R | chr6: 30654392-30654934 | N_Shore |
| cg18079653 | chr10: 72648646 | PCBD1.TSS200 | R | chr10: 72647738-72648317 | S_Shore |
| cg01200905 | chr16: 52480264 | TOX3.GENEBODY | R | | |
| cg07543967 | chr19: 49055443 | SULT2B1.EXON1 | R | | |
| cg08389577 | chr6: 3229379 | TUBB2B.TSS1500 | R | chr6: 3227025-3229688 | Island |
| cg13796218 | chr1: 11750663 | MAD2L2.UTR5 | R | chr1: 11750701-11752651 | N_Shore |
| cg15927865 | chr10: 104159579 | NFKB2.GENEBODY | F | chr10: 104159096-104159536 | S_Shore |
| cg17677988 | chr4: 10687769 | CLNK.TSS1500 | F | | |
| cg02333237 | chr14: 50802971 | CDKL1.GENEBODY | R | | |
| cg05467716 | chr17: 62774079 | LOC146880.GENEBODY | F | chr17: 62774366-62777797 | N_Shore |
| cg00452755 | chr1: 28864993 | SNHG3-RCC1.GENEBODY | R | | |
| cg14423168 | chr8: 41400312 | GINS4.UTR3 | R | | |
| cg14373410 | chr14: 81426234 | TSHR.GENEBODY | R | | |
| cg01033600 | chr19: 7964703 | LRRC8E.GENEBODY | F | chr19: 7964439-7964704 | Island |
| cg16616765 | chr2: 197456534 | HECW2.UTR5 | R | chr2: 197457392-197458522 | N_Shore |
| cg08093568 | chr7: 27626595 | HIBADH.GENEBODY | F | | |
| cg05152503 | chr20: 30407098 | MYLK2.TSS200 | R | chr20: 30410547-30410832 | N_Shelf |
| cg22970357 | chr2: 119600408 | EN1.UTR3 | R | chr2: 119599458-119600966 | Island |
| cg00791283 | chr12: 79498966 | SYT1.UTR5 | R | | |
| cg26181372 | chr9: 104198262 | ALDOB.TSS1500 | R | | |
| cg22780475 | chr19: 45281526 | CBLC.EXON1 | F | chr19: 45281133-45281355 | S_Shore |
| cg09336589 | chr17: 7107939 | DLG4.GENEBODY | F | chr17: 7108304-7108654 | N_Shore |
| cg25976170 | chr22: 29707585 | GAS2L1.UTR3 | F | chr22: 29707479-29707797 | Island |
| cg07557423 | chr4: 42657903 | ATP8A1.GENEBODY | F | chr4: 42658278-42659550 | N_Shore |
| cg07127888 | chr8: 41400789 | GINS4.UTR3 | F | | |
| cg16293838 | chr1: 6330521 | ACOT7.GENEBODY | F | | |
| cg17397069 | chr5: 156110742 | SGCD.GENEBODY | R | | |
| cg15580167 | chr2: 175629219 | CHRNA1.TSS200 | F | | |
| cg14414874 | chr9: 126157693 | DENND1A.GENEBODY | F | chr9: 126154226-126154562 | S_Shelf |
| cg03167954 | chr22: 29707782 | GAS2L1.UTR3 | R | chr22: 29707479-29707797 | Island |
| cg22976732 | chr22: 28191914 | MN1.GENEBODY | F | chr22: 28192793-28198592 | N_Shore |
| cg00927494 | chr11: 69924230 | ANO1.TSS200 | R | chr11: 69924338-69925197 | N_Shore |
| cg21661983 | chr12: 72353490 | TPH2.GENEBODY | F | | |
| cg00664609 | chr16: 2198075 | RAB26.TSS1500 | R | chr16: 2198455-2199129 | N_Shore |
| cg23924526 | chr2: 45998548 | PRKCE.GENEBODY | R | | |
| cg03180426 | chr9: 139964156 | C9orf140.GENEBODY | R | chr9: 139964155-139965284 | Island |
| cg10153942 | chr19: 54982500 | CDC42EP5.UTR5 | R | chr19: 54982388-54982897 | Island |
| cg11282156 | chr3: 63428840 | SYNPR.UTR5 | F | | |
| cg18003659 | chr2: 175629267 | CHRNA1.TSS200 | R | | |
| cg01862650 | chr5: 143619194 | KCTD16.GENEBODY | R | | |
| cg01828813 | chr6: 33132705 | COL11A2.GENEBODY | R | chr6: 33129291-33129718 | S_Shelf |
| cg19887293 | chr6: 33132693 | COL11A2.GENEBODY | R | chr6: 33129291-33129718 | S_Shelf |
| cg25736198 | chr12: 72345225 | TPH2.GENEBODY | R | | |
| cg01159294 | chr5: 143763289 | KCTD16.GENEBODY | F | | |
| cg23009046 | chr11: 119600595 | PVRL1.TSS1500 | R | chr11: 119598382-119600157 | S_Shore |
| cg26548251 | chr7: 147591462 | MIR548F3.TSS1500 | F | | |
| cg00177243 | chr10: 18434068 | CACNB2.GENEBODY | F | chr10: 18429125-18430234 | S_Shelf |
| cg03050127 | chr17: 59413660 | BCAS3.GENEBODY | R | | |
| cg26357587 | chr11: 120110882 | POU2F3.TSS200 | R | chr11: 120110497-120110719 | S_Shore |
| cg02965092 | chr12: 72347052 | TPH2.GENEBODY | F | | |
| cg07961632 | chr1: 27668421 | SYTL1.TSS200 | R | | |
| cg10380643 | chr1: 1356764 | LOC441869.TSS200 | R | chr1: 1355159-1356000 | S_Shore |
| cg17790159 | chr6: 33133083 | COL11A2.GENEBODY | R | chr6: 33129291-33129718 | S_Shelf |
| cg14154859 | chr1: 220702826 | MARK1.GENEBODY | F | chr1: 220701507-220702348 | S_Shore |
| cg10120778 | chr7: 42018795 | GLI3.GENEBODY | F | | |
| cg15700587 | chr7: 147065665 | MIR548I4.GENEBODY | R | | |
| cg00333034 | chr3: 183948058 | VWA5B2.TSS1500 | F | chr3: 183947040-183948801 | Island |
| cg00789347 | chr16: 2039030 | SYNGR3.TSS1500 | F | chr16: 2040034-2040628 | N_Shore |
| cg01507577 | chr7: 146484609 | CNTNAP2.GENEBODY | R | | |
| cg02379560 | chr4: 40859280 | APBB2.GENEBODY | F | chr4: 40858884-40859162 | S_Shore |
| cg04917391 | chr4: 40859259 | APBB2.GENEBODY | F | chr4: 40858884-40859162 | S_Shore |
| cg05894734 | chr4: 40859249 | APBB2.GENEBODY | F | chr4: 40858884-40859162 | S_Shore |
| cg06485940 | chr4: 40859293 | APBB2.GENEBODY | F | chr4: 40858884-40859162 | S_Shore |
| cg07035001 | chr7: 146321053 | CNTNAP2.GENEBODY | F | | |
| cg12455762 | chr4: 40859095 | APBB2.GENEBODY | F | chr4: 40858884-40859162 | Island |
| cg14230238 | chr19: 6066871 | RFX2.UTR5 | R | | |
| cg14248352 | chr7: 146820536 | CNTNAP2.GENEBODY | R | | |
| cg14652038 | chr2: 222343519 | EPHA4.GENEBODY | R | | |
| cg16364495 | chr4: 40859251 | APBB2.GENEBODY | F | chr4: 40858884-40859162 | S_Shore |
| cg22519702 | chr5: 147204189 | SPINK1.UTR3 | R | | |
| cg23735712 | chr4: 40858984 | APBB2.GENEBODY | F | chr4: 40858884-40859162 | Island |
| cg23889772 | chr19: 10077324 | COL5A3.GENEBODY | F | chr19: 10077031-10077444 | Island |
| cg25649655 | chr8: 145692504 | CYHR1.TSS1500 | F | chr8: 145690605-145692505 | Island |
| cg26244738 | chr4: 40859344 | APBB2.GENEBODY | R | chr4: 40858884-40859162 | S_Shore |
| cg26470314 | chr16: 2039411 | SYNGR3.TSS1500 | R | chr16: 2040034-2040628 | N_Shore |
| cg01657995 | chr6: 31804883 | C6orf48.UTR5 | F | chr6: 31802598-31802823 | S_Shelf |
| cg02269097 | chr12: 49487944 | DHH.GENEBODY | R | chr12: 49487963-49488202 | N_Shore |
| cg03392565 | chr1: 212464932 | PPP2R5A.GENEBODY | R | | |
| cg03957440 | chr1: 214209324 | PROX1.UTR3 | R | | |
| cg09050775 | chr12: 46762708 | SLC38A2.GENEBODY | R | chr12: 46765134-46766633 | N_Shelf |
| cg11526866 | chr1: 20813614 | CAMK2N1.TSS1500 | F | chr1: 20810462-20813511 | S_Shore |
| cg13109300 | chr12: 49488587 | DHH.UTR5 | F | chr12: 49487963-49488202 | S_Shore |
| cg16151451 | chr11: 849153 | TSPAN4.UTR5 | F | chr11: 849087-850982 | Island |
| cg16405211 | chr7: 146897691 | CNTNAP2.GENEBODY | R | | |
| cg17490790 | chr19: 10125043 | RDH8.GENEBODY | R | chr19: 10120750-10121231 | S_Shelf |
| cg18804920 | chr7: 146572638 | CNTNAP2.GENEBODY | R | | |
| cg19075717 | chr17: 48670940 | CACNA1G.GENEBODY | R | | |
| cg24127874 | chr2: 239149859 | HES6.TSS1500 | F | chr2: 239147592-239149900 | Island |
| cg04303901 | chr12: 97886312 | RMST.GENEBODY | F | | |
| cg04695027 | chr11: 65793986 | CATSPER1.EXON1 | R | chr11: 65789909-65790168 | S_Shelf |
| cg04865716 | chr19: 2290843 | LINGO3.GENEBODY | R | chr19: 2289895-2291904 | Island |
| cg05582979 | chr4: 10686610 | CLNK.TSS1500 | F | | |
| cg06093379 | chr17: 44896080 | WNT3.UTR5 | R | chr17: 44895800-44897025 | Island |
| cg08384314 | chr6: 30711680 | IER3.UTR3 | R | chr6: 30710307-30712440 | Island |
| cg09267188 | chr8: 97340225 | PTDSS1.GENEBODY | R | | |
| cg12724192 | chr4: 82392157 | RASGEF1B.UTR5 | R | chr4: 82392610-82393426 | N_Shore |
| cg13067221 | chr14: 23449582 | JUB.GENEBODY | F | chr14: 23449886-23452174 | N_Shore |
| cg16406078 | chr20: 825634 | FAM110A.GENEBODY | F | chr20: 825267-826276 | Island |
| cg17971747 | chr15: 45410236 | DUOXA1.GENEBODY | F | chr15: 45408573-45409528 | S_Shore |
| cg22942704 | chr1: 20813574 | CAMK2N1.TSS1500 | R | chr1: 20810462-20813511 | S_Shore |
| cg25151498 | chr3: 185815992 | ETV5.GENEBODY | F | | |
| cg25564319 | chr3: 63558416 | SYNPR.GENEBODY | F | | |
| cg27190967 | chr16: 2039391 | SYNGR3.TSS1500 | F | chr16: 2040034-2040628 | N_Shore |
| cg01920182 | chr12: 23738340 | SOX5.TSS1500 | R | | |
| cg03595580 | chr7: 97880848 | TECPR1.UTR5 | F | chr7: 97881132-97881906 | N_Shore |
| cg04993130 | chr11: 726759 | EPS8L2.GENEBODY | R | chr11: 725596-726870 | Island |
| cg06736134 | chr5: 147040725 | JAKMIP2.GENEBODY | R | | |
| cg09248054 | chr1: 969257 | AGRN.GENEBODY | F | chr1: 967966-970238 | Island |
| cg11565786 | chr5: 31944970 | PDZD2.GENEBODY | R | | |
| cg16769791 | chr8: 85178030 | RALYL.UTR5 | R | | |
| cg19741167 | chr2: 86263270 | POLR1A.GENEBODY | R | | |
| cg25225073 | chr14: 90528983 | KCNK13.GENEBODY | F | chr14: 90526696-90528951 | S_Shore |
| cg26683639 | chr6: 30653755 | KIAA1949.GENEBODY | F | chr6: 30654392-30654934 | N_Shore |
| cg07788437 | chr19: 1799952 | ATP8B3.GENEBODY | R | chr19: 1799934-1800248 | Island |
| cg11207515 | chr7: 146904205 | CNTNAP2.GENEBODY | R | | |
| cg11540204 | chr1: 60391699 | CYP2J2.GENEBODY | R | chr1: 60392272-60392583 | N_Shore |
| cg11799006 | chr5: 147101975 | JAKMIP2.UTR5 | R | | |
| cg13369952 | chr5: 147024959 | JAKMIP2.GENEBODY | F | | |
| cg14074581 | chr19: 36232218 | TMEM149.UTR5 | F | chr19: 36231186-36232219 | Island |
| cg25183613 | chr3: 111717557 | TAGLN3.TSS200 | F | | |
| cg00967013 | chr13: 24735754 | SPATA13.UTR5 | F | chr13: 24734771-24735538 | S_Shore |
| cg02120463 | chr22: 39639003 | PDGFB.GENEBODY | F | chr22: 39638219-39640966 | Island |
| cg02578321 | chr1: 247275872 | C1orf229.TSS200 | R | chr1: 247274585-247275757 | S_Shore |
| cg03415518 | chr12: 97885729 | RMST.GENEBODY | R | | |
| cg04513006 | chr16: 68270252 | ESRP2.TSS200 | F | chr16: 68269010-68272143 | Island |
| cg05825085 | chr16: 86379872 | LOC732275.TSS1500 | F | | |
| cg06185146 | chr3: 63428305 | SYNPR.GENEBODY | F | | |
| cg08669018 | chr4: 82392280 | RASGEF1B.UTR5 | R | chr4: 82392610-82393426 | N_Shore |
| cg09473576 | chr1: 20813647 | CAMK2N1.TSS1500 | F | chr1: 20810462-20813511 | S_Shore |
| cg10083593 | chr11: 66049855 | CNIH2.GENEBODY | R | chr11: 66049750-66050229 | Island |
| cg11408784 | chr17: 7166293 | CLDN7.TSS1500 | F | chr17: 7164285-7166245 | S_Shore |
| cg11876912 | chr3: 63602009 | SYNPR.UTR3 | F | | |
| cg13250388 | chr22: 39641078 | PDGFB.TSS200 | R | chr22: 39638219-39640966 | S_Shore |
| cg14615553 | chr5: 147093466 | JAKMIP2.UTR5 | F | | |
| cg15149374 | chr3: 19576826 | KCNH8.UTR3 | R | | |
| cg15225810 | chr19: 750790 | C19orf21.TSS1500 | F | chr19: 746300-747161 | S_Shelf |
| cg17075538 | chr19: 15306892 | NOTCH3.GENEBODY | R | chr19: 15306243-15307111 | Island |
| cg17410295 | chr12: 109729809 | FOXN4.GENEBODY | F | chr12: 109729572-109729826 | Island |
| cg17475643 | chr8: 10261972 | MSRA.GENEBODY | F | chr8: 10261900-10262222 | Island |
| cg19464419 | chr6: 94064439 | EPHA7.GENEBODY | R | | |
| cg20391895 | chr4: 10686394 | CLNK.TSS200 | F | | |
| cg24238564 | chr11: 65779343 | CST6.TSS200 | F | chr11: 65779311-65779767 | Island |
| cg26939195 | chr6: 32077020 | TNXB.UTR5 | F | | |
| cg00026290 | chr4: 10686554 | CLNK.TSS200 | F | | |
| cg02785561 | chr8: 10262221 | MSRA.GENEBODY | F | chr8: 10261900-10262222 | Island |
| cg04221461 | chr1: 243877024 | AKT3.GENEBODY | R | | |
| cg05082805 | chr7: 146489513 | CNTNAP2.GENEBODY | F | | |
| cg09433131 | chr8: 73480179 | KCNB2.GENEBODY | F | | |
| cg11652360 | chr6: 33160120 | COL11A2.UTR5 | R | chr6: 33159991-33160263 | Island |
| cg19311889 | chr12: 57483793 | NAB2.GENEBODY | R | chr12: 57480797-57483116 | S_Shore |
| cg00835193 | chr19: 2291780 | LINGO3.UTR5 | R | chr19: 2289895-2291904 | Island |
| cg01708411 | chr12: 57631839 | NDUFA4L2.UTR5 | R | chr12: 57631932-57634578 | N_Shore |
| cg02958126 | chr11: 62370246 | EML3.GENEBODY | F | chr11: 62368454-62370491 | Island |
| cg02989255 | chr6: 32063774 | TNXB.GENEBODY | F | chr6: 32063533-32065044 | Island |
| cg03525956 | chr3: 174720287 | NAALADL2.GENEBODY | F | | |
| cg06191495 | chr2: 234264163 | DGKD.GENEBODY | R | | |
| cg11809958 | chr12: 32654929 | FGD4.TSS200 | R | | |
| cg11845241 | chr7: 36960377 | MIR1200.TSS1500 | R | | |
| cg13179469 | chr13: 107185675 | EFNB2.GENEBODY | F | chr13: 107186468-107189024 | N_Shore |
| cg18349863 | chr6: 29912713 | HLA-A.GENEBODY | F | chr6: 29910202-29911367 | S_Shore |
| cg22138393 | chr8: 97340270 | PTDSS1.GENEBODY | F | | |
| cg26726230 | chr17: 79225857 | SLC38A10.GENEBODY | F | | |
| cg26961166 | chr8: 73687160 | KCNB2.GENEBODY | F | | |
| cg26999775 | chr14: 105347803 | KIAA0284.GENEBODY | R | chr14: 105350177-105350925 | N_Shelf |
| cg00433917 | chr19: 7915517 | EVI5L.GENEBODY | F | chr19: 7913751-7913968 | S_Shore |
| cg01422274 | chr11: 849088 | TSPAN4.UTR5 | R | chr11: 849087-850982 | Island |
| cg02245020 | chr7: 143580147 | FAM115A.UTR5 | R | chr7: 143582125-143582610 | N_Shore |
| cg04545963 | chr14: 35872393 | NFKBIA.GENEBODY | F | chr14: 35873047-35873990 | N_Shore |
| cg07384913 | chr3: 63637118 | SNTN.TSS1500 | F | | |
| cg07706352 | chr19: 10077245 | COL5A3.GENEBODY | R | chr19: 10077031-10077444 | Island |
| cg08279008 | chr2: 86263224 | POLR1A.GENEBODY | F | | |
| cg10144863 | chr8: 53235234 | ST18.UTR5 | R | | |
| cg11743827 | chr3: 185080905 | MAP3K13.TSS200 | R | | |
| cg13720581 | chr6: 32077017 | TNXB.UTR5 | F | | |
| cg15148573 | chr7: 103498566 | RELN.GENEBODY | F | | |
| cg16078836 | chr3: 185081374 | MAP3K13.UTR5 | R | | |
| cg16226037 | chr3: 175338261 | NAALADL2.GENEBODY | R | | |
| cg16715129 | chr11: 10477821 | AMPD3.EXON1 | F | | |
| cg19418951 | chr3: 185080816 | MAP3K13.TSS200 | R | | |
| cg24519146 | chr6: 33160318 | COL11A2.TSS200 | F | chr6: 33160945-33161337 | N_Shore |
| cg25264265 | chr3: 185080997 | MAP3K13.EXON1 | R | | |
| cg25479682 | chr3: 185081403 | MAP3K13.UTR5 | R | | |
| cg26334804 | chr19: 41724208 | AXL.TSS1500 | R | | |
| cg27287254 | chr6: 33160067 | COL11A2.UTR5 | F | chr6: 33159991-33160263 | Island |
| cg27633342 | chr15: 52588279 | MYO5C.TSS1500 | R | chr15: 52587353-52588172 | S_Shore |
| cg00037056 | chr4: 956607 | DGKQ.GENEBODY | F | chr4: 960505-960836 | N_Shelf |
| cg01484141 | chr17: 1624157 | WDR81.GENEBODY | R | chr17: 1627745-1629897 | N_Shelf |
| cg01679081 | chr5: 60139131 | ELOVL7.UTR5 | F | chr5: 60139359-60140155 | N_Shore |
| cg01886125 | chr2: 110054030 | SH3RF3.GENEBODY | F | | |
| cg03508107 | chr17: 47654357 | NXPH3.GENEBODY | F | chr17: 47653211-47654369 | Island |
| cg03676636 | chr4: 99064102 | C4orf37.GENEBODY | R | chr4: 99064602-99064805 | N_Shore |
| cg03850035 | chr2: 222367110 | EPHA4.GENEBODY | R | | |
| cg04083466 | chr6: 25753732 | SLC17A4.TSS1500 | F | | |
| cg05396397 | chr1: 11908164 | NPPA.TSS1500 | R | | |
| cg07107420 | chr7: 8010894 | GLCCI1.GENEBODY | F | chr7: 8008097-8009495 | S_Shore |
| cg10242279 | chr4: 10666415 | CLNK.GENEBODY | R | | |
| cg12286569 | chr19: 18722620 | TMEM59L.TSS1500 | R | chr19: 18722358-18724001 | Island |
| cg13298116 | chr11: 62369859 | EML3.UTR3 | R | chr11: 62368454-62370491 | Island |
| cg14848284 | chr2: 73143293 | EMX1.TSS1500 | R | chr2: 73143055-73148260 | Island |
| cg16686429 | chr19: 46284983 | DMPK.GENEBODY | F | chr19: 46282544-46283127 | S_Shore |
| cg18164281 | chr20: 2797721 | LOC100288797.UTR3 | R | chr20: 2801639-2802135 | N_Shelf |
| cg18776945 | chr5: 147024842 | JAKMIP2.GENEBODY | R | | |
| cg19250002 | chr22: 20460068 | RIMBP3.EXON1 | F | chr22: 20459267-20461473 | Island |
| cg19268434 | chr6: 32076967 | TNXB.UTR5 | F | | |
| cg22076422 | chr3: 63399168 | SYNPR.GENEBODY | R | | |
| cg22168512 | chr18: 74695224 | MBP.GENEBODY | F | chr18: 74691262-74691466 | S_Shelf |
| cg26248155 | chr17: 7165849 | CLDN7.TSS200 | R | chr17: 7164285-7166245 | Island |
| cg26574680 | chr1: 235811860 | GNG4.UTR5 | F | chr1: 235811841-235814066 | Island |
| cg26627129 | chr19: 49552470 | CGB8.TSS200 | F | | |
| cg00102996 | chr9: 92220617 | GADD45G.GENEBODY | R | chr9: 92219771-92221411 | Island |
| cg01589998 | chr12: 109729478 | FOXN4.GENEBODY | R | chr12: 109729572-109729826 | N_Shore |
| cg01994275 | chr7: 8480586 | NXPH1.GENEBODY | F | chr7: 8481974-8482762 | N_Shore |
| cg04013592 | chr3: 63263076 | SYNPR.TSS1500 | F | chr3: 63263989-63264205 | N_Shore |
| cg05848863 | chr7: 16794078 | TSPAN13.GENEBODY | R | chr7: 16793188-16794100 | Island |
| cg09649438 | chr10: 81904160 | PLAC9.GENEBODY | R | | |
| cg13223402 | chr2: 47797133 | KCNK12.EXON1 | R | chr2: 47796923-47799166 | Island |
| cg13827782 | chr9: 93377568 | DIRAS2.UTR5 | R | | |
| cg13978352 | chr6: 30908706 | DPCR1.TSS200 | R | | |
| cg14750475 | chr19: 50139654 | RRAS.GENEBODY | F | chr19: 50143223-50143975 | N_Shelf |
| cg15954675 | chr3: 63542363 | SYNPR.GENEBODY | R | | |
| cg19195419 | chr19: 1799935 | ATP8B3.GENEBODY | R | chr19: 1799934-1800248 | Island |
| cg20083442 | chr8: 73600575 | KCNB2.GENEBODY | R | | |
| cg20353496 | chr20: 825268 | FAM110A.TSS200 | R | chr20: 825267-826276 | Island |
| cg21574349 | chr3: 185080951 | MAP3K13.TSS200 | F | | |
| cg23422361 | chr10: 135143617 | CALY.UTR5 | F | chr10: 135143373-135143580 | S_Shore |
| cg23463608 | chr19: 2607757 | GNG7.UTR5 | F | chr19: 2607725-2607967 | Island |
| cg27233151 | chr4: 82057467 | PRKG2.GENEBODY | R | | |
| cg02365303 | chr5: 147052498 | JAKMIP2.UTR5 | F | | |
| cg08694699 | chr16: 68270282 | ESRP2.TSS200 | F | chr16: 68269010-68272143 | Island |
| cg20668718 | chr5: 146991927 | JAKMIP2.GENEBODY | F | | |
| cg20818262 | chr8: 73480145 | KCNB2.GENEBODY | R | | |
| cg22459924 | chr19: 2607850 | GNG7.UTR5 | F | chr19: 2607725-2607967 | Island |
| cg11610754 | chr19: 18722595 | TMEM59L.TSS1500 | R | chr19: 18722358-18724001 | Island |
| cg12053155 | chr13: 107186476 | EFNB2.GENEBODY | R | chr13: 107186468-107189024 | Island |
| cg12232903 | chr4: 10686413 | CLNK.TSS200 | F | | |
| cg14995475 | chr19: 6534774 | TNFSF9.GENEBODY | R | chr19: 6534759-6535079 | Island |
| cg16661000 | chr15: 89437710 | HAPLN3.UTR5 | F | chr15: 89438176-89438850 | N_Shore |
| cg17208427 | chr16: 68679556 | CDH3.GENEBODY | R | chr16: 68678686-68680149 | Island |
| cg18721026 | chr3: 183948313 | VWA5B2.TSS200 | F | chr3: 183947040-183948801 | Island |
| cg00090619 | chr6: 33160286 | COL11A2.TSS200 | F | chr6: 33160945-33161337 | N_Shore |
| cg00106565 | chr5: 146784171 | DPYSL3.GENEBODY | F | | |
| cg00303541 | chr3: 51741280 | GRM2.UTR5 | R | chr3: 51740740-51741413 | Island |
| cg01177388 | chr6: 32812704 | PSMB8.UTR5 | F | chr6: 32811494-32811839 | S_Shore |
| cg01294327 | chr19: 2291373 | LINGO3.GENEBODY | R | chr19: 2289895-2291904 | Island |
| cg02688752 | chr5: 76115309 | F2RL1.GENEBODY | R | chr5: 76115510-76116089 | N_Shore |
| cg02969706 | chr2: 6072283 | LOC150622.TSS1500 | R | | |
| cg03225210 | chr7: 143579951 | FAM115A.UTR5 | R | chr7: 143582125-143582610 | N_Shelf |
| cg04458159 | chr6: 29856538 | HLA-H.GENEBODY | F | chr6: 29855295-29856565 | Island |
| cg04872123 | chr6: 32049699 | TNXB.GENEBODY | R | chr6: 32046815-32047094 | S_Shelf |
| cg05057976 | chr11: 65779667 | CST6.EXON1 | F | chr11: 65779311-65779767 | Island |
| cg06321045 | chr6: 31088343 | CDSN.TSS200 | F | | |
| cg06529264 | chr13: 54887288 | MIR1297.TSS1500 | F | | |
| cg07435445 | chr14: 103396379 | AMN.GENEBODY | R | chr14: 103394397-103397070 | Island |
| cg08551725 | chr7: 30029776 | SCRN1.TSS1500 | F | chr7: 30028518-30029822 | Island |
| cg11606261 | chr12: 53775336 | SP1.UTR5 | R | chr12: 53774336-53774603 | S_Shore |
| cg11794801 | chr8: 10262020 | MSRA.GENEBODY | R | chr8: 10261900-10262222 | Island |
| cg11941546 | chr17: 7165842 | CLDN7.TSS200 | R | chr17: 7164285-7166245 | Island |
| cg13083674 | chr19: 55953372 | SHISA7.GENEBODY | R | chr19: 55953371-55954641 | Island |
| cg13470920 | chr19: 6772831 | VAV1.EXON1 | F | | |
| cg13723983 | chr9: 132383362 | C9orf50.TSS1500 | F | chr9: 132382432-132383004 | S_Shore |
| cg13851989 | chr9: 14720244 | CER1.GENEBODY | R | | |
| cg13984492 | chr9: 104031694 | LPPR1.GENEBODY | R | | |
| cg15705054 | chr1: 154927709 | PBXIP1.UTR5 | R | | |
| cg17930034 | chr14: 65016602 | C14orf50.TSS200 | R | chr14: 65016516-65016909 | Island |
| cg20114890 | chr6: 29856430 | HLA-H.GENEBODY | R | chr6: 29855295-29856565 | Island |
| cg21476000 | chr12: 54430684 | HOXC4.UTR5 | F | chr12: 54427025-54428709 | S_Shore |
| cg21552300 | chr2: 175629224 | CHRNA1.TSS200 | F | | |
| cg23292266 | chr19: 11285128 | KANK2.GENEBODY | R | chr19: 11285122-11285332 | Island |
| cg23517605 | chr6: 3228365 | TUBB2B.TSS1500 | F | chr6: 3227025-3229688 | Island |
| cg24735489 | chr6: 31088352 | CDSN.TSS200 | F | | |
| cg25036095 | chr4: 82392410 | RASGEF1B.UTR5 | F | chr4: 82392610-82393426 | N_Shore |
| cg25580825 | chr19: 10116808 | COL5A3.GENEBODY | F | chr19: 10120750-10121231 | N_Shelf |
| cg25661884 | chr19: 10124267 | RDH8.EXON1 | R | chr19: 10120750-10121231 | S_Shelf |
| cg25751198 | chr12: 109729573 | FOXN4.GENEBODY | R | chr12: 109729572-109729826 | Island |
| cg00378510 | chr19: 2291020 | LINGO3.GENEBODY | F | chr19: 2289895-2291904 | Island |
| cg17259886 | chr5: 147040667 | JAKMIP2.GENEBODY | F | | |
| cg00961418 | chr11: 8616172 | STK33.TSS1500 | F | chr11: 8615397-8615889 | S_Shore |
| cg01218350 | chr2: 210517441 | MAP2.UTR5 | R | | |
| cg01974715 | chr19: 41724129 | AXL.TSS1500 | F | | |
| cg02374996 | chr9: 92220771 | GADD45G.GENEBODY | F | chr9: 92219771-92221411 | Island |
| cg03250393 | chr19: 41735107 | AXL.GENEBODY | F | chr19: 41732006-41732556 | S_Shelf |
| cg03436967 | chr7: 147207052 | MIR54814.GENEBODY | F | | |
| cg03549571 | chr12: 49488198 | DHH.EXON1 | R | chr12: 49487963-49488202 | Island |
| cg03832506 | chrX: 53286187 | IQSEC2.GENEBODY | R | chrX: 53283829-53284038 | S_Shelf |
| cg04328477 | chr16: 68270258 | ESRP2.TSS200 | F | chr16: 68269010-68272143 | Island |
| cg05171933 | chr5: 147205668 | SPINK1.GENEBODY | F | | |
| cg06193043 | chr1: 11908199 | NPPA.TSS1500 | F | | |
| cg06205574 | chr12: 23858674 | SOX5.GENEBODY | F | | |
| cg06671964 | chr6: 32052105 | TNXB.GENEBODY | F | chr6: 32055067-32055601 | N_Shelf |
| cg06723863 | chr16: 68270129 | ESRP2.EXON1 | F | chr16: 68269010-68272143 | Island |
| cg07896312 | chr8: 22409062 | SORBS3.TSS200 | R | chr8: 22408672-22409635 | Island |
| cg08416875 | chr6: 119669226 | MAN1A1.GENEBODY | R | chr6: 119669589-119671424 | N_Shore |
| cg08575922 | chr12: 49488082 | DHH.EXON1 | R | chr12: 49487963-49488202 | Island |
| cg09807148 | chr17: 34122069 | MMP28.GENEBODY | F | chr17: 34122024-34122801 | Island |
| cg09990600 | chr7: 1596103 | TMEM184A.TSS200 | R | | |
| cg10030250 | chr16: 55540152 | MMP2.UTR3 | F | chr16: 55542920-55543274 | N_Shelf |
| cg10131879 | chr7: 8011594 | GLCCI1.GENEBODY | F | chr7: 8008097-8009495 | S_Shelf |
| cg10688790 | chr7: 147591961 | MIR548F3.TSS1500 | R | | |
| cg11384924 | chr6: 33160224 | COL11A2.UTR5 | F | chr6: 33159991-33160263 | Island |
| cg11664251 | chr2: 149634581 | KIF5C.GENEBODY | R | chr2: 149632682-149633882 | S_Shore |
| cg11989343 | chr13: 53287193 | LECT1.GENEBODY | R | | |
| cg12444861 | chr6: 144386275 | PLAGL1.TSS1500 | F | chr6: 144384894-144385888 | S_Shore |
| cg12626076 | chr15: 92399195 | SLCO3A1.GENEBODY | F | chr15: 92396013-92397682 | S_Shore |
| cg13586420 | chr6: 33160262 | COL11A2.TSS200 | F | chr6: 33159991-33160263 | Island |
| cg13969788 | chr9: 35689475 | TPM2.GENEBODY | F | chr9: 35689610-35691101 | N_Shore |
| cg14011070 | chr1: 235811811 | GNG4.UTR5 | R | chr1: 235811841-235814066 | N_Shore |
| cg14627974 | chr7: 147188028 | MIR54814.GENEBODY | F | | |
| cg15558616 | chr3: 173630182 | NLGN1.GENEBODY | R | | |
| cg15678504 | chr8: 10262159 | MSRA.GENEBODY | R | chr8: 10261900-10262222 | Island |
| cg15712559 | chr20: 58180616 | PHACTR3.GENEBODY | F | chr20: 58179808-58180787 | Island |
| cg15982299 | chr6: 31097078 | PSORS1C1.UTR5 | F | | |
| cg16014168 | chr12: 109729677 | FOXN4.GENEBODY | F | chr12: 109729572-109729826 | Island |
| cg16911349 | chr19: 10406935 | ICAM5.GENEBODY | R | chr19: 10406934-10407342 | Island |
| cg18216587 | chr7: 146019420 | CNTNAP2.GENEBODY | F | | |
| cg18516816 | chr19: 30165591 | PLEKHF1.UTR3 | F | chr19: 30164768-30165556 | S_Shore |
| cg19047707 | chr2: 233351525 | ECEL1.UTR5 | R | chr2: 233350278-233352974 | Island |
| cg20010135 | chr16: 30996822 | HSD3B7.UTR5 | F | | |
| cg20330126 | chr22: 36125053 | APOL5.UTR3 | F | | |
| cg21446725 | chr12: 49740996 | DNAJC22.TSS200 | R | chr12: 49738680-49740841 | S_Shore |
| cg22857356 | chr19: 8398590 | KANK3.GENEBODY | R | chr19: 8397958-8400461 | Island |
| cg23097961 | chr19: 49055412 | SULT2B1.TSS200 | F | | |
| cg23344395 | chr3: 192634002 | C3orf59.GENEBODY | R | chr3: 192634309-192635967 | N_Shore |
| cg23623622 | chr6: 30139880 | TRIM15.GENEBODY | F | chr6: 30139718-30140263 | Island |
| cg23764900 | chr11: 8616102 | STK33.TSS1500 | R | chr11: 8615397-8615889 | S_Shore |
| cg23883945 | chr7: 36749772 | AOAH.GENEBODY | F | | |
| cg23937993 | chr11: 10477847 | AMPD3.EXON1 | F | | |
| cg24021846 | chr7: 103556529 | RELN.GENEBODY | F | | |
| cg24952408 | chr6: 29696264 | LOC285830.GENEBODY | F | | |
| cg25934371 | chr3: 63427866 | SYNPR.GENEBODY | F | | |
| cg27023555 | chr2: 233351735 | ECEL1.UTR5 | R | chr2: 233350278-233352974 | Island |
| cg27108362 | chr12: 49488554 | DHH.UTR5 | F | chr12: 49487963-49488202 | S_Shore |
| cg27181765 | chr6: 33160250 | COL11A2.TSS200 | F | chr6: 33159991-33160263 | Island |
| cg01635061 | chr6: 33160234 | COL11A2.UTR5 | F | chr6: 33159991-33160263 | Island |
| cg02582213 | chr16: 89441654 | ANKRD11.UTR5 | F | chr16: 89438503-89438850 | S_Shelf |
| cg02628840 | chr21: 46860579 | COL18A1.GENEBODY | R | chr21: 46860366-46860628 | Island |
| cg06712559 | chr1: 968395 | AGRN.GENEBODY | F | chr1: 967966-970238 | Island |
| cg09235583 | chr6: 30711141 | IER3.UTR3 | F | chr6: 30710307-30712440 | Island |
| cg20931195 | chr17: 708009 | NXN.GENEBODY | F | chr17: 708319-708598 | N_Shore |
| cg25549619 | chr5: 147154246 | JAKMIP2.UTR5 | F | | |
| cg27480819 | chr12: 23686149 | SOX5.UTR3 | R | | |
| cg00201445 | chr3: 192634310 | C3orf59.GENEBODY | R | chr3: 192634309-192635967 | Island |
| cg00650953 | chr7: 147500503 | CNTNAP2.GENEBODY | R | | |
| cg00698688 | chr19: 49055432 | SULT2B1.EXON1 | F | | |
| cg00965643 | chr1: 32674817 | DCDC2B.EXON1 | F | chr1: 32670569-32671885 | S_Shelf |
| cg00974598 | chr19: 44223902 | IRGC.GENEBODY | R | chr19: 44222858-44223943 | Island |
| cg01030534 | chr7: 143579698 | FAM115A.UTR5 | F | chr7: 143582125-143582610 | N_Shelf |
| cg01243297 | chr6: 33160292 | COL11A2.TSS200 | F | chr6: 33160945-33161337 | N_Shore |
| cg02633149 | chr17: 81042782 | METRNL.GENEBODY | R | chr17: 81042626-81043244 | Island |
| cg03039843 | chr19: 49055390 | SULT2B1.TSS200 | R | | |
| cg03485217 | chr4: 10686300 | CLNK.EXON1 | R | | |
| cg03607415 | chr5: 89881642 | GPR98.GENEBODY | R | | |
| cg03761471 | chr7: 143077178 | ZYX.TSS1500 | F | chr7: 143077469-143079169 | N_Shore |
| cg03936135 | chr16: 86371248 | LOC732275.GENEBODY | F | | |
| cg04601383 | chr19: 6713459 | C3.GENEBODY | R | chr19: 6710625-6710828 | S_Shelf |
| cg05681757 | chr12: 32655034 | FGD4.TSS200 | R | | |
| cg06822340 | chr7: 147355839 | CNTNAP2.GENEBODY | R | | |
| cg06852032 | chr17: 25976191 | LGALS9.GENEBODY | F | | |
| cg07001963 | chr10: 44879465 | CXCL12.GENEBODY | R | chr10: 44879714-44882391 | N_Shore |
| cg07095230 | chr17: 59480730 | TBX2.GENEBODY | F | chr17: 59473060-59483266 | Island |
| cg07152605 | chr1: 164793520 | PBX1.GENEBODY | R | | |
| cg07270851 | chr11: 65779332 | CST6.TSS200 | F | chr11: 65779311-65779767 | Island |
| cg07287698 | chr12: 97957454 | MIR135A2.TSS200 | F | | |
| cg08787401 | chr2: 222383520 | EPHA4.GENEBODY | F | | |
| cg09225457 | chr7: 8479893 | NXPH1.GENEBODY | R | chr7: 8481974-8482762 | N_Shelf |
| cg09932135 | chr1: 11750569 | MAD2L2.UTR5 | F | chr1: 11750701-11752651 | N_Shore |
| cg11904429 | chr19: 6592554 | CD70.TSS1500 | F | chr19: 6590340-6591072 | S_Shore |
| cg12197421 | chr6: 30653736 | KIAA1949.GENEBODY | F | chr6: 30654392-30654934 | N_Shore |
| cg12747860 | chr6: 3229027 | TUBB2B.TSS1500 | R | chr6: 3227025-3229688 | Island |
| cg12805420 | chr17: 7165840 | CLDN7.TSS200 | R | chr17: 7164285-7166245 | Island |
| cg12967164 | chr7: 1753637 | ELFN1.UTR5 | F | chr7: 1753446-1753706 | Island |
| cg13094252 | chr8: 104153753 | C8orf56.TSS200 | R | chr8: 104152726-104153390 | S_Shore |
| cg13673837 | chr19: 15306670 | NOTCH3.GENEBODY | F | chr19: 15306243-15307111 | Island |
| cg14282850 | chr10: 123924917 | TACC2.GENEBODY | F | chr10: 123922850-123923542 | S_Shore |
| cg14347177 | chr11: 65779330 | CST6.TSS200 | F | chr11: 65779311-65779767 | Island |
| cg14574369 | chr12: 57483631 | NAB2.GENEBODY | R | chr12: 57480797-57483116 | S_Shore |
| cg14663984 | chr1: 969042 | AGRN.GENEBODY | F | chr1: 967966-970238 | Island |
| cg15633699 | chr1: 1356728 | LOC441869.TSS200 | F | chr1: 1355159-1356000 | S_Shore |
| cg16663570 | chr8: 22014724 | LGI3.TSS1500 | F | chr8: 22014152-22014559 | S_Shore |
| cg16713727 | chr1: 16062720 | SLC25A34.TSS200 | R | | |
| cg17928920 | chr1: 968558 | AGRN.GENEBODY | R | chr1: 967966-970238 | Island |
| cg18425731 | chr2: 158184641 | ERMN.TSS1500 | F | | |
| cg19092045 | chr13: 54887369 | MIR1297.TSS1500 | R | | |
| cg19244428 | chr6: 3229233 | TUBB2B.TSS1500 | R | chr6: 3227025-3229688 | Island |
| cg19791630 | chr19: 6772811 | VAV1.UTR5 | F | | |
| cg20308663 | chr13: 100523121 | CLYBL.GENEBODY | F | | |
| cg20561300 | chr15: 93189780 | FAM174B.GENEBODY | R | | |
| cg21131024 | chr8: 104153643 | C8orf56.TSS200 | F | chr8: 104152726-104153390 | S_Shore |
| cg21422016 | chr7: 1753659 | ELFN1.UTR5 | R | chr7: 1753446-1753706 | Island |
| cg22156128 | chr3: 10327453 | GHRL.GENEBODY | R | | |
| cg22431093 | chr12: 32654992 | FGD4.TSS200 | R | | |
| cg23049291 | chr1: 165204745 | LMX1A.GENEBODY | F | chr1: 165204501-165205507 | Island |
| cg26080567 | chr21: 37536137 | DOPEY2.TSS1500 | R | | |
| cg26190885 | chr19: 45409005 | APOE.TSS200 | F | chr19: 45411720-45412600 | N_Shelf |
| cg26638341 | chr19: 1800126 | ATP8B3.GENEBODY | F | chr19: 1799934-1800248 | Island |
| cg26737640 | chr19: 6587584 | CD70.GENEBODY | F | chr19: 6590340-6591072 | N_Shelf |
| cg26946905 | chr16: 89441595 | ANKRD11.UTR5 | F | chr16: 89438503-89438850 | S_Shelf |
| cg26993132 | chr16: 89239499 | CDH15.GENEBODY | F | chr16: 89237944-89238331 | S_Shore |
| cg27082921 | chr19: 1800150 | ATP8B3.GENEBODY | R | chr19: 1799934-1800248 | Island |
| cg02349785 | chr11: 65778967 | CST6.TSS1500 | R | chr11: 65779311-65779767 | N_Shore |
| cg02576092 | chr1: 27668556 | SYTL1.EXON1 | R | | |
| cg04327181 | chr11: 65779526 | CST6.EXON1 | F | chr11: 65779311-65779767 | Island |
| cg04626868 | chr12: 23999034 | SOX5.GENEBODY | R | | |
| cg05601974 | chr7: 150941389 | SMARCD3.GENEBODY | F | chr7: 150942304-150943033 | N_Shore |
| cg09482611 | chr11: 65779263 | CST6.TSS200 | R | chr11: 65779311-65779767 | N_Shore |
| cg14452346 | chr5: 147212032 | SPINK1.TSS1500 | F | | |
| cg19296556 | chr19: 1802918 | ATP8B3.GENEBODY | F | chr19: 1799934-1800248 | S_Shelf |
| cg24478779 | chr2: 6110966 | LOC150622.GENEBODY | R | | |
| cg04016660 | chr16: 85075232 | KIAA0513.UTR5 | R | | |
| cg00107982 | chr22: 37813098 | ELFN2.UTR5 | F | chr22: 37815803-37816477 | N_Shelf |
| cg00135874 | chr2: 210493473 | MAP2.UTR5 | R | | |
| cg00464796 | chr15: 53852614 | WDR72.GENEBODY | F | | |
| cg01084435 | chr16: 58535556 | NDRG4.GENEBODY | F | chr16: 58535040-58535596 | Island |
| cg03992611 | chr15: 92397873 | SLCO3A1.GENEBODY | R | chr15: 92396013-92397682 | S_Shore |
| cg04080448 | chr12: 97924358 | RMST.GENEBODY | F | | |
| cg05393025 | chr1: 20141781 | RNF186.TSS200 | R | | |
| cg06189653 | chr5: 176873332 | PRR7.TSS1500 | R | chr5: 176873349-176874737 | N_Shore |
| cg06705685 | chr2: 8116961 | LOC339788.TSS200 | F | | |
| cg08151612 | chr19: 49055438 | SULT2B1.EXON1 | R | | |
| cg08966208 | chr11: 1769409 | HCCA2.GENEBODY | F | chr11: 1768979-1770712 | Island |
| cg11406274 | chr6: 29856564 | HLA-H.GENEBODY | F | chr6: 29855295-29856565 | Island |
| cg11736020 | chr6: 30651511 | KIAA1949.GENEBODY | R | chr6: 30654392-30654934 | N_Shelf |
| cg11920519 | chr20: 33135025 | MAP1LC3A.UTR5 | R | | |
| cg12570246 | chr7: 36959356 | MIR1200.TSS1500 | F | | |
| cg12732715 | chr1: 148929740 | LOC645166.TSS1500 | F | | |
| cg12781794 | chr11: 118505740 | PHLDB1.GENEBODY | F | chr11: 118505508-118505741 | Island |
| cg13729891 | chr17: 7108468 | DLG4.GENEBODY | F | chr17: 7108304-7108654 | Island |
| cg13951572 | chr8: 22409000 | SORBS3.TSS1500 | F | chr8: 22408672-22409635 | Island |
| cg14089943 | chr10: 103990076 | PITX3.UTR3 | R | chr10: 103990213-103991829 | N_Shore |
| cg14279361 | chr19: 6721955 | C3.TSS1500 | R | | |
| cg14397690 | chr15: 60688948 | ANXA2.UTR5 | F | chr15: 60689749-60690430 | N_Shore |
| cg14492061 | chr3: 63637129 | SNTN.TSS1500 | R | | |
| cg14899716 | chr1: 42846987 | RIMKLA.GENEBODY | F | chr1: 42845978-42846988 | Island |
| cg15249911 | chr19: 41634046 | CYP2F1.UTR3 | R | chr19: 41633776-41634047 | Island |
| cg15507500 | chr19: 17605696 | SLC27A1.GENEBODY | F | chr19: 17607983-17608406 | N_Shelf |
| cg16094352 | chr14: 24642317 | REC8.GENEBODY | R | chr14: 24641053-24642220 | S_Shore |
| cg16318112 | chr1: 969254 | AGRN.GENEBODY | F | chr1: 967966-970238 | Island |
| cg16706099 | chr11: 100590197 | FLJ32810.GENEBODY | F | | |
| cg17058616 | chr17: 73102145 | SLC16A5.UTR3 | R | chr17: 73105229-73106667 | N_Shelf |
| cg17610929 | chr2: 220379044 | ACCN4.UTR5 | R | chr2: 220377743-220377946 | S_Shore |
| cg17826168 | chr19: 6464103 | CRB3.TSS200 | F | chr19: 6463991-6464780 | Island |
| cg17945153 | chr6: 41978288 | CCND3.UTR5 | R | | |
| cg19260567 | chr7: 147303836 | CNTNAP2.GENEBODY | F | | |
| cg20855296 | chr17: 16255560 | CENPV.GENEBODY | R | chr17: 16255618-16257148 | N_Shore |
| cg21061582 | chr18: 44498047 | PIAS2.TSS1500 | F | chr18: 44496910-44497832 | S_Shore |
| cg21238061 | chr9: 104036809 | LPPR1.GENEBODY | R | | |
| cg22365276 | chr15: 60688622 | ANXA2.UTR5 | R | chr15: 60689749-60690430 | N_Shore |
| cg22376856 | chr1: 204643556 | LRRN2.UTR5 | R | | |
| cg22862634 | chr11: 62369728 | EML3.UTR3 | R | chr11: 62368454-62370491 | Island |
| cg23214395 | chr1: 20141778 | RNF186.TSS200 | R | | |
| cg23265500 | chr14: 65016591 | C14orf50.TSS200 | R | chr14: 65016516-65016909 | Island |
| cg23912509 | chr12: 97957406 | MIR135A2.TSS200 | F | | |
| cg24556026 | chr9: 36258971 | GNE.TSS1500 | F | chr9: 36258170-36258886 | S_Shore |
| cg24723883 | chr19: 2608495 | GNG7.UTR5 | F | chr19: 2607725-2607967 | S_Shore |
| cg25124058 | chr12: 97886218 | RMST.GENEBODY | F | | |
| cg26633373 | chr19: 54377836 | MYADM.UTR3 | R | chr19: 54377172-54377441 | S_Shore |
| cg26660800 | chr17: 59480926 | TBX2.GENEBODY | F | chr17: 59473060-59483266 | Island |
| cg00187437 | chr6: 31089157 | CDSN.TSS1500 | R | | |
| cg00294550 | chr10: 62144786 | ANK3.GENEBODY | F | | |
| cg02447471 | chr7: 95537015 | DYNC1I1.GENEBODY | F | | |
| cg11243173 | chr12: 23999045 | SOX5.GENEBODY | R | | |
| cg14894216 | chr11: 65794352 | CATSPER1.TSS1500 | R | | |
| cg17295739 | chr6: 32049513 | TNXB.GENEBODY | R | chr6: 32046815-32047094 | S_Shelf |
| cg24283921 | chr11: 10477461 | AMPD3.TSS200 | F | | |
| cg26070020 | chr1: 165201385 | LMX1A.GENEBODY | F | chr1: 165204501-165205507 | N_Shelf |
| cg27248041 | chr6: 93992464 | EPHA7.GENEBODY | F | | |
| cg03514404 | chr7: 143579665 | FAM115A.UTR5 | F | chr7: 143582125-143582610 | N_Shelf |
| cg00151914 | chr17: 34058550 | RASL10B.TSS200 | R | chr17: 34058549-34058778 | Island |
| cg00721771 | chr3: 59903821 | FHIT.GENEBODY | F | | |
| cg00784153 | chr19: 10077007 | COL5A3.GENEBODY | R | chr19: 10077031-10077444 | N_Shore |
| cg00957688 | chr1: 36183672 | C1orf216.UTR5 | F | chr1: 36184248-36184774 | N_Shore |
| cg01110620 | chr12: 113573312 | RASAL1.UTR5 | F | chr12: 113572977-113573758 | Island |
| cg01538731 | chr16: 87757018 | KLHDC4.GENEBODY | F | chr16: 87754008-87754285 | S_Shelf |
| cg01794802 | chr6: 31096931 | PSORS1C1.UTR5 | R | | |
| cg02004418 | chr2: 45236740 | SIX2.TSS200 | R | chr2: 45235511-45237792 | Island |
| cg02227919 | chr2: 171569132 | LOC440925.GENEBODY | R | chr2: 171569877-171573904 | N_Shore |
| cg02849309 | chr6: 144386280 | PLAGL1.TSS1500 | F | chr6: 144384894-144385888 | S_Shore |
| cg02916272 | chr11: 1769443 | HCCA2.GENEBODY | F | chr11: 1768979-1770712 | Island |
| cg03153115 | chr19: 2604559 | GNG7.UTR5 | F | chr19: 2607725-2607967 | N_Shelf |
| cg03207915 | chr6: 119669112 | MAN1A1.GENEBODY | F | chr6: 119669589-119671424 | N_Shore |
| cg03546687 | chr16: 2198080 | RAB26.TSS1500 | R | chr16: 2198455-2199129 | N_Shore |
| cg04396998 | chr19: 49376722 | PPP1R15A.GENEBODY | R | chr19: 49375484-49375928 | S_Shore |
| cg04475868 | chr16: 86380594 | LOC732275.TSS1500 | F | | |
| cg04742671 | chr19: 54781049 | LILRB2.GENEBODY | R | | |
| cg05301811 | chr1: 20813666 | CAMK2N1.TSS1500 | F | chr1: 20810462-20813511 | S_Shore |
| cg05484788 | chr20: 58180357 | PHACTR3.GENEBODY | F | chr20: 58179808-58180787 | Island |
| cg06303936 | chr3: 172711298 | SPATA16.GENEBODY | F | | |
| cg07629125 | chr13: 58299015 | PCDH17.GENEBODY | R | | |
| cg08247297 | chr21: 46917541 | COL18A1.GENEBODY | F | chr21: 46918188-46918410 | N_Shore |
| cg08269485 | chr11: 1769382 | HCCA2.GENEBODY | F | chr11: 1768979-1770712 | Island |
| cg08550882 | chr11: 12308354 | MICALCL.TSS200 | R | | |
| cg09708598 | chr13: 54887314 | MIR1297.TSS1500 | F | | |
| cg10303653 | chr6: 32049516 | TNXB.GENEBODY | R | chr6: 32046815-32047094 | S_Shelf |
| cg10560147 | chr11: 65779335 | CST6.TSS200 | F | chr11: 65779311-65779767 | Island |
| cg12432038 | chr1: 247275735 | C1orf229.TSS200 | R | chr1: 247274585-247275757 | Island |
| cg13064679 | chr6: 32049953 | TNXB.GENEBODY | R | chr6: 32046815-32047094 | S_Shelf |
| cg13095737 | chr5: 177034964 | B4GALT7.GENEBODY | R | chr5: 177035351-177035660 | N_Shore |
| cg15005677 | chr11: 45918197 | MAPK8IP1.GENEBODY | R | chr11: 45921387-45922167 | N_Shelf |
| cg15144888 | chr11: 128760367 | KCNJ5.TSS1500 | F | chr11: 128760441-128761233 | N_Shore |
| cg15323252 | chr8: 22436420 | PDLIM2.TSS1500 | R | chr8: 22436295-22437076 | Island |
| cg16317181 | chr10: 85954559 | PCDH21.EXON1 | R | chr10: 85954199-85955303 | Island |
| cg17765324 | chr12: 16359122 | SLC15A5.GENEBODY | F | | |
| cg18320766 | chr19: 55598782 | EPS8L1.GENEBODY | R | chr19: 55597977-55598887 | Island |
| cg19455335 | chr8: 22457658 | C8orf58.GENEBODY | R | chr8: 22457123-22457753 | Island |
| cg19961803 | chr11: 65554928 | OVOL1.EXON1 | F | chr11: 65553749-65555573 | Island |
| cg20383948 | chr21: 46898137 | COL18A1.GENEBODY | R | chr21: 46897841-46898158 | Island |
| cg21475065 | chr7: 147336337 | CNTNAP2.GENEBODY | R | | |
| cg22153696 | chr4: 186389425 | CCDC110.GENEBODY | F | chr4: 186392574-186393184 | N_Shelf |
| cg22287800 | chr7: 8634300 | NXPH1.GENEBODY | F | | |
| cg24042496 | chr11: 117497929 | DSCAML1.GENEBODY | R | | |
| cg24990327 | chr7: 147591897 | MIR548F3.TSS1500 | R | | |
| cg25317941 | chr2: 233351153 | ECEL1.GENEBODY | R | chr2: 233350278-233352974 | Island |
| cg26104986 | chr11: 75275303 | SERPINH1.UTR5 | F | chr11: 75272950-75273616 | S_Shore |
| cg27579097 | chr16: 734844 | JMJD8.TSS1500 | R | chr16: 729438-735815 | Island |
| cg04486528 | chr7: 147727995 | MIR548F3.GENEBODY | F | | |
| cg07047570 | chr20: 20350813 | INSM1.EXON1 | F | chr20: 20344400-20350605 | S_Shore |
| cg07912147 | chr12: 26138851 | RASSF8.UTR5 | R | | |
| cg09246103 | chr1: 8456466 | RERE.UTR5 | R | | |
| cg09247061 | chr16: 88571844 | ZFPM1.GENEBODY | F | chr16: 88573285-88573489 | N_Shore |
| cg09365529 | chr1: 75140289 | C1orf173.TSS1500 | F | | |
| cg14950855 | chr12: 49740781 | DNAJC22.TSS1500 | F | chr12: 49738680-49740841 | Island |
| cg15262242 | chr17: 47301741 | PHOSPHO1.GENEBODY | R | chr17: 47301545-47302477 | Island |
| cg15802396 | chr17: 7108305 | DLG4.GENEBODY | R | chr17: 7108304-7108654 | Island |
| cg16182479 | chr17: 41832466 | SOST.UTR3 | R | chr17: 41832717-41833195 | N_Shore |
| cg16630229 | chr6: 33160604 | COL11A2.TSS1500 | F | chr6: 33160945-33161337 | N_Shore |
| cg18395327 | chr2: 220379109 | ACCN4.EXON1 | R | chr2: 220377743-220377946 | S_Shore |
| cg19822502 | chr6: 97456581 | KLHL32.GENEBODY | F | | |
| cg22189307 | chr11: 61061699 | VWCE.GENEBODY | R | chr11: 61061751-61063063 | N_Shore |
| cg23619910 | chr11: 10477452 | AMPD3.TSS200 | R | | |
| cg24155984 | chr5: 66127678 | MAST4.GENEBODY | F | | |
| cg26578983 | chr16: 85075249 | KIAA0513.UTR5 | R | | |
| cg00691368 | chr6: 30909820 | DPCR1.GENEBODY | F | | |
| cg00988129 | chr8: 75232556 | JPH1.GENEBODY | R | chr8: 75232649-75234101 | N_Shore |
| cg03716590 | chr7: 95435309 | DYNC1I1.GENEBODY | R | | |
| cg04770813 | chr6: 33160297 | COL11A2.TSS200 | F | chr6: 33160945-33161337 | N_Shore |
| cg05273302 | chr19: 2095560 | C19orf36.TSS1500 | F | chr19: 2095395-2095727 | Island |
| cg06212760 | chr1: 46664937 | POMGNT1.GENEBODY | R | chr1: 46668821-46669258 | N_Shelf |
| cg07308243 | chr16: 85074963 | KIAA0513.UTR5 | R | | |
| cg07409292 | chr4: 90228685 | GPRIN3.UTR5 | R | chr4: 90228714-90229010 | N_Shore |
| cg08531687 | chr5: 89968232 | GPR98.GENEBODY | F | | |
| cg09335647 | chr7: 147097440 | MIR548I4.GENEBODY | F | | |
| cg10332715 | chr1: 245017736 | HNRNPU.UTR3 | F | | |
| cg10383447 | chr16: 58535093 | NDRG4.GENEBODY | F | chr16: 58535040-58535596 | Island |
| cg10632765 | chr19: 6534916 | TNFSF9.GENEBODY | F | chr19: 6534759-6535079 | Island |
| cg12046414 | chr2: 220379199 | ACCN4.EXON1 | R | chr2: 220377743-220377946 | S_Shore |
| cg12489353 | chr19: 48231499 | EHD2.GENEBODY | F | chr19: 48229072-48229507 | S_Shore |
| cg13455326 | chr19: 10043341 | OLFM2.GENEBODY | R | chr19: 10046824-10047067 | N_Shelf |
| cg14098532 | chr3: 111717874 | TAGLN3.TSS200 | F | | |
| cg14478768 | chr19: 1083429 | HMHA1.GENEBODY | F | chr19: 1082576-1083430 | Island |
| cg15939253 | chr11: 66049932 | CNIH2.GENEBODY | F | chr11: 66049750-66050229 | Island |
| cg16678522 | chr20: 32149963 | CBFA2T2.GENEBODY | F | | |
| cg17831934 | chr7: 147264850 | CNTNAP2.GENEBODY | R | | |
| cg17894318 | chr3: 181441384 | SOX2OT.GENEBODY | F | chr3: 181444409-181445000 | N_Shelf |
| cg18512948 | chr14: 24641706 | REC8.EXON1 | R | chr14: 24641053-24642220 | Island |
| cg19506253 | chr2: 158301839 | CYTIP.TSS1500 | F | | |
| cg19702383 | chr12: 97885575 | RMST.GENEBODY | F | | |
| cg19799744 | chr6: 46138963 | ENPP5.TSS1500 | R | | |
| cg19924334 | chr20: 62197682 | PRIC285.GENEBODY | F | chr20: 62193966-62198985 | Island |
| cg20716515 | chr19: 44172263 | PLAUR.GENEBODY | F | chr19: 44172916-44173252 | N_Shore |
| cg21389753 | chr17: 59480674 | TBX2.GENEBODY | F | chr17: 59473060-59483266 | Island |
| cg22881677 | chr12: 63195695 | PPM1H.GENEBODY | R | chr12: 63195657-63195909 | Island |
| cg24743778 | chr2: 220379058 | ACCN4.UTR5 | R | chr2: 220377743-220377946 | S_Shore |
| cg25242557 | chr11: 31833264 | PAX6.TSS1500 | R | chr11: 31831620-31839038 | Island |
| cg25540824 | chr6: 30139686 | TRIM15.GENEBODY | F | chr6: 30139718-30140263 | N_Shore |
| cg25587092 | chr6: 32076906 | TNXB.UTR5 | R | | |
| cg27125631 | chr17: 47654653 | NXPH3.GENEBODY | F | chr17: 47652789-47653078 | S_Shore |
| cg00936309 | chr6: 33141824 | COL11A2.GENEBODY | F | | |
| cg01116568 | chr11: 10476529 | AMPD3.TSS200 | R | chr11: 10472000-10472857 | S_Shelf |
| cg01594262 | chr19: 38806145 | YIF1B.GENEBODY | R | chr19: 38806485-38806747 | N_Shore |
| cg02355653 | chr6: 29710830 | LOC285830.GENEBODY | R | | |
| cg02452416 | chr6: 33160450 | COL11A2.TSS1500 | R | chr6: 33160945-33161337 | N_Shore |
| cg03054529 | chr7: 30029938 | SCRN1.TSS1500 | F | chr7: 30028518-30029822 | S_Shore |
| cg04138361 | chr6: 33160304 | COL11A2.TSS200 | F | chr6: 33160945-33161337 | N_Shore |
| cg04403415 | chr12: 124991087 | NCOR2.UTR5 | F | | |
| cg07387813 | chr3: 40499408 | RPL14.GENEBODY | R | chr3: 40498639-40499260 | S_Shore |
| cg08858649 | chr6: 30139903 | TRIM15.GENEBODY | R | chr6: 30139718-30140263 | Island |
| cg09537107 | chr19: 48897008 | GRIN2D.TSS1500 | F | chr19: 48896890-48897199 | Island |
| cg12049787 | chr19: 45409080 | APOE.EXON1 | R | chr19: 45411720-45412600 | N_Shelf |
| cg12077433 | chr3: 159600838 | SCHIP1.GENEBODY | F | | |
| cg12108912 | chr2: 120435949 | TMEM177.TSS1500 | R | chr2: 120436530-120437010 | N_Shore |
| cg12643732 | chr5: 691580 | TPPP.UTR5 | F | chr5: 692681-694377 | N_Shore |
| cg12776156 | chr11: 848044 | TSPAN4.UTR5 | F | chr11: 849087-850982 | N_Shore |
| cg13212283 | chr12: 57632120 | NDUFA4L2.UTR5 | F | chr12: 57631932-57634578 | Island |
| cg14507088 | chr9: 130690399 | PIP5KL1.GENEBODY | R | chr9: 130689378-130690215 | S_Shore |
| cg15622556 | chr1: 12251820 | TNFRSF1B.GENEBODY | F | chr1: 12251777-12251985 | Island |
| cg16458153 | chr6: 33160192 | COL11A2.UTR5 | R | chr6: 33159991-33160263 | Island |
| cg16523839 | chr19: 10077134 | COL5A3.GENEBODY | R | chr19: 10077031-10077444 | Island |
| cg16825133 | chr19: 675672 | FSTL3.TSS1500 | R | chr19: 675671-675881 | Island |
| cg16860712 | chr19: 48244461 | EHD2.GENEBODY | F | chr19: 48244150-48244693 | Island |
| cg18636319 | chr4: 26492206 | CCKAR.TSS200 | R | | |
| cg18788725 | chr19: 45512122 | RELB.GENEBODY | R | chr19: 45515163-45515560 | N_Shelf |
| cg19776793 | chr17: 79225974 | SLC38A10.GENEBODY | F | | |
| cg20333836 | chr1: 156215418 | PAQR6.GENEBODY | R | chr1: 156215368-156215899 | Island |
| cg20639396 | chr6: 128839906 | PTPRK.GENEBODY | R | chr6: 128840158-128842126 | N_Shore |
| cg20672284 | chr12: 23998921 | SOX5.GENEBODY | R | | |
| cg21009420 | chr7: 146484504 | CNTNAP2.GENEBODY | R | | |
| cg23337031 | chr2: 236416064 | AGAP1.GENEBODY | F | | |
| cg23627353 | chr8: 102669360 | GRHL2.GENEBODY | F | | |
| cg26674800 | chr17: 7108653 | DLG4.GENEBODY | F | chr17: 7108304-7108654 | Island |
| cg26853536 | chr12: 125399964 | UBC.TSS1500 | F | chr12: 125398558-125399899 | S_Shore |
| cg00126034 | chr17: 44896162 | WNT3.TSS200 | F | chr17: 44895800-44897025 | Island |
| cg00758881 | chr16: 58534681 | NDRG4.GENEBODY | R | chr16: 58535040-58535596 | N_Shore |
| cg01029347 | chr14: 77606335 | ZDHHC22.UTR5 | R | chr14: 77605885-77608304 | Island |
| cg02010738 | chr19: 2095466 | C19orf36.TSS1500 | R | chr19: 2095395-2095727 | Island |
| cg03300078 | chr11: 1769522 | HCCA2.GENEBODY | F | chr11: 1768979-1770712 | Island |
| cg04663692 | chr12: 97885669 | RMST.GENEBODY | R | | |
| cg04694991 | chr10: 97219665 | SORBS1.UTR5 | F | | |
| cg06998765 | chr14: 75389618 | RPS6KL1.TSS1500 | F | chr14: 75389712-75390133 | N_Shore |
| cg07304692 | chr2: 100939045 | LONRF2.UTR5 | R | chr2: 100937779-100939059 | Island |
| cg07775371 | chr16: 1922155 | C16orf73.UTR5 | F | chr16: 1921897-1922426 | Island |
| cg09271709 | chr2: 50863603 | NRXN1.GENEBODY | R | | |
| cg09327981 | chr7: 95455393 | DYNC111.GENEBODY | F | | |
| cg09750510 | chr6: 25761690 | SLC17A4.UTR5 | R | | |
| cg10098010 | chr6: 138483709 | KIAA1244.GENEBODY | F | chr6: 138482534-138483710 | Island |
| cg10599438 | chr19: 844589 | PRTN3.GENEBODY | F | chr19: 846011-846435 | N_Shore |
| cg11565299 | chr6: 30139478 | TRIM15.GENEBODY | F | chr6: 30139718-30140263 | N_Shore |
| cg11775828 | chr2: 169100915 | STK39.GENEBODY | F | chr2: 169102950-169104454 | N_Shelf |
| cg12281657 | chr14: 65016595 | C14orf50.TSS200 | R | chr14: 65016516-65016909 | Island |
| cg12317456 | chr16: 55512868 | MMP2.TSS1500 | R | chr16: 55513220-55513526 | N_Shore |
| cg12699599 | chr15: 40643142 | PHGR1.TSS200 | R | | |
| cg12934382 | chr3: 51741135 | GRM2.EXON1 | F | chr3: 51740740-51741413 | Island |
| cg13313836 | chr15: 60687284 | ANXA2.UTR5 | R | chr15: 60689749-60690430 | N_Shelf |
| cg14307471 | chr18: 31432117 | NOL4.UTR3 | F | | |
| cg15174248 | chr5: 149669674 | CAMK2A.TSS1500 | F | | |
| cg17176573 | chr11: 120110887 | POU2F3.TSS200 | R | chr11: 120110497-120110719 | S_Shore |
| cg19419650 | chr19: 7929643 | EVI5L.UTR3 | F | chr19: 7926590-7928752 | S_Shore |
| cg19519964 | chr2: 223290012 | SGPP2.GENEBODY | F | chr2: 223288922-223290013 | Island |
| cg19722656 | chr6: 119669372 | MAN1A1.GENEBODY | R | chr6: 119669589-119671424 | N_Shore |
| cg20971970 | chr12: 57632495 | NDUFA4L2.UTR5 | R | chr12: 57631932-57634578 | Island |
| cg21492961 | chr5: 90036436 | GPR98.GENEBODY | R | | |
| cg21759280 | chr19: 6534936 | TNFSF9.GENEBODY | F | chr19: 6534759-6535079 | Island |
| cg21958743 | chr12: 79832188 | SYT1.GENEBODY | F | | |
| cg23084895 | chr1: 214162854 | PROX1.UTR5 | R | chr1: 214160798-214161034 | S_Shore |
| cg25746092 | chr6: 31081617 | PSORS1C1.TSS1500 | F | | |
| cg26576206 | chr19: 1064938 | ABCA7.GENEBODY | F | chr19: 1064897-1065191 | Island |
| cg26839863 | chr10: 21814793 | C10orf140.TSS200 | R | chr10: 21814786-21815716 | Island |
| cg26995690 | chr13: 36346376 | DCLK1.UTR3 | F | | |
| cg00293644 | chr1: 32674341 | DCDC2B.TSS1500 | R | chr1: 32670569-32671885 | S_Shelf |
| cg00407245 | chr19: 55598874 | EPS8L1.GENEBODY | F | chr19: 55597977-55598887 | Island |
| cg00848007 | chr12: 68553396 | IFNG.EXON1 | F | | |
| cg00956142 | chr1: 28765031 | PHACTR4.GENEBODY | F | | |
| cg01666490 | chr8: 102984290 | NCALD.UTR5 | R | | |
| cg01783706 | chr12: 49739991 | DNAJC22.TSS1500 | R | chr12: 49738680-49740841 | Island |
| cg01902845 | chr1: 214170993 | PROX1.GENEBODY | R | | |
| cg02275396 | chr19: 15554549 | WIZ.GENEBODY | R | chr19: 15550849-15551059 | S_Shelf |
| cg03186999 | chr17: 7165420 | CLDN7.UTR5 | F | chr17: 7164285-7166245 | Island |
| cg05257528 | chr19: 846179 | PRTN3.GENEBODY | F | chr19: 846011-846435 | Island |
| cg06371583 | chr19: 2581343 | GNG7.UTR5 | F | chr19: 2578956-2579746 | S_Shore |
| cg06764736 | chr12: 23998997 | SOX5.GENEBODY | F | | |
| cg07299645 | chr7: 147256263 | MIR548I4.TSS1500 | F | | |
| cg07475922 | chr19: 10122694 | RDH8.TSS1500 | R | chr19: 10120750-10121231 | S_Shore |
| cg08753391 | chr6: 32049622 | TNXB.GENEBODY | R | chr6: 32046815-32047094 | S_Shelf |
| cg08897422 | chr7: 8666902 | NXPH1.GENEBODY | F | | |
| cg09588360 | chr2: 54785795 | SPTBN1.UTR5 | R | chr2: 54785026-54785969 | Island |
| cg10357885 | chr19: 10122000 | COL5A3.TSS1500 | R | chr19: 10120750-10121231 | S_Shore |
| cg11289677 | chr6: 32049829 | TNXB.GENEBODY | R | chr6: 32046815-32047094 | S_Shelf |
| cg11620475 | chr5: 135529232 | LOC389332.TSS1500 | R | chr5: 135527056-135528978 | S_Shore |
| cg11742103 | chr11: 62369870 | EML3.UTR3 | R | chr11: 62368454-62370491 | Island |
| cg13500113 | chr2: 216947149 | TMEM169.UTR5 | R | chr2: 216946324-216946734 | S_Shore |
| cg13711424 | chr14: 65017091 | C14orf50.GENEBODY | R | chr14: 65016516-65016909 | S_Shore |
| cg14025053 | chr8: 104153637 | C8orf56.TSS200 | F | chr8: 104152726-104153390 | S_Shore |
| cg15159069 | chr3: 192634089 | C3orf59.GENEBODY | F | chr3: 192634309-192635967 | N_Shore |
| cg16573136 | chr12: 49740639 | DNAJC22.TSS1500 | F | chr12: 49738680-49740841 | Island |
| cg16663341 | chr18: 42327562 | SETBP1.GENEBODY | R | chr18: 42324454-42324966 | S_Shelf |
| cg17617491 | chr19: 2607726 | GNG7.UTR5 | R | chr19: 2607725-2607967 | Island |
| cg17714703 | chr19: 4912221 | UHRF1.GENEBODY | R | chr19: 4909262-4910256 | S_Shore |
| cg18010131 | chr11: 10476620 | AMPD3.TSS200 | F | chr11: 10472000-10472857 | S_Shelf |
| cg18168101 | chr8: 104153627 | C8orf56.TSS200 | F | chr8: 104152726-104153390 | S_Shore |
| cg18544974 | chr3: 132842314 | TMEM108.UTR5 | R | | |
| cg18716083 | chr5: 111506418 | EPB41L4A.GENEBODY | F | | |
| cg19132462 | chr11: 10476608 | AMPD3.TSS200 | R | chr11: 10472000-10472857 | S_Shelf |
| cg19776942 | chr1: 32714164 | FAM167B.GENEBODY | F | chr1: 32713918-32714186 | Island |
| cg19865770 | chr1: 221510889 | LOC400804.TSS1500 | F | | |
| cg21171335 | chr12: 122356390 | WDR66.TSS200 | F | chr12: 122356315-122356853 | Island |
| cg21213853 | chr3: 51741473 | GRM2.UTR5 | F | chr3: 51740740-51741413 | S_Shore |
| cg21869609 | chr19: 2291613 | LINGO3.GENEBODY | R | chr19: 2289895-2291904 | Island |
| cg22494035 | chr15: 74537819 | CCDC33.GENEBODY | R | chr15: 74537809-74538114 | Island |
| cg22699676 | chr2: 171569187 | LOC440925.GENEBODY | R | chr2: 171569877-171573904 | N_Shore |
| cg23601905 | chr17: 17661060 | RAI1.UTR5 | F | | |
| cg23635599 | chr19: 45281006 | CBLC.TSS200 | F | chr19: 45281133-45281355 | N_Shore |
| cg23816247 | chr8: 104153827 | BAALC.GENEBODY | F | chr8: 104152726-104153390 | S_Shore |
| cg24852713 | chr19: 1083423 | HMHA1.GENEBODY | F | chr19: 1082576-1083430 | Island |
| cg27103348 | chr21: 46321529 | ITGB2.GENEBODY | R | chr21: 46321397-46321644 | Island |
| cg27273866 | chr12: 23919997 | SOX5.GENEBODY | R | | |
| cg01337207 | chr6: 32063835 | TNXB.GENEBODY | F | chr6: 32063533-32065044 | Island |
| cg02027561 | chr6: 126110790 | NCOA7.TSS1500 | R | chr6: 126111537-126112480 | N_Shore |
| cg03601354 | chr8: 22408966 | SORBS3.TSS1500 | F | chr8: 22408672-22409635 | Island |
| cg04015907 | chr20: 825489 | FAM110A.GENEBODY | F | chr20: 825267-826276 | Island |
| cg05830220 | chr16: 87757033 | KLHDC4.GENEBODY | F | chr16: 87754008-87754285 | S_Shelf |
| cg05957504 | chr5: 124014639 | ZNF608.GENEBODY | F | | |
| cg06198398 | chr18: 44526707 | KATNAL2.TSS200 | R | chr18: 44526866-44527137 | N_Shore |
| cg07312445 | chr4: 40752151 | NSUN7.UTR5 | F | chr4: 40751842-40752493 | Island |
| cg07524919 | chr6: 32063901 | TNXB.GENEBODY | F | chr6: 32063533-32065044 | Island |
| cg10578482 | chr8: 145111298 | OPLAH.GENEBODY | R | chr8: 145111297-145111564 | Island |
| cg10625931 | chr12: 57632417 | NDUFA4L2.UTR5 | R | chr12: 57631932-57634578 | Island |
| cg11632652 | chr3: 174648902 | NAALADL2.GENEBODY | F | | |
| cg13788097 | chr3: 62467612 | CADPS.GENEBODY | F | | |
| cg14188106 | chr6: 32063895 | TNXB.GENEBODY | F | chr6: 32063533-32065044 | Island |
| cg15075267 | chr19: 15306332 | NOTCH3.GENEBODY | F | chr19: 15306243-15307111 | Island |
| cg15857329 | chr8: 144653570 | C8orf73.GENEBODY | F | chr8: 144653774-144653976 | N_Shore |
| cg16314401 | chr2: 73144481 | EMX1.TSS200 | R | chr2: 73143055-73148260 | Island |
| cg16924391 | chr6: 29719411 | IFITM4P.TSS1500 | F | chr6: 29720381-29721101 | N_Shore |
| cg16953031 | chr3: 175064540 | NAALADL2.GENEBODY | F | | |
| cg17693604 | chr19: 3046805 | TLE2.UTR5 | F | chr19: 3047224-3047444 | N_Shore |
| cg18612786 | chr19: 45281236 | CBLC.EXON1 | R | chr19: 45281133-45281355 | Island |
| cg20502039 | chr7: 95804328 | SLC25A13.GENEBODY | F | | |
| cg23288962 | chr13: 35517516 | NBEA.GENEBODY | R | | |
| cg23563443 | chr8: 22408989 | SORBS3.TSS1500 | F | chr8: 22408672-22409635 | Island |
| cg24836242 | chr6: 30908579 | DPCR1.TSS200 | F | | |
| cg24847554 | chr3: 57530606 | DNAH12.TSS1500 | F | | |
| cg27501759 | chr3: 8809715 | OXTR.GENEBODY | F | chr3: 8808961-8811280 | Island |
| cg00378218 | chr17: 1546841 | SCARF1.GENEBODY | F | chr17: 1546742-1547324 | Island |
| cg00637695 | chr5: 121759771 | SNCAIP.GENEBODY | R | | |
| cg01101058 | chr14: 70235157 | SFRS5.GENEBODY | F | chr14: 70233570-70234668 | S_Shore |
| cg01645995 | chr7: 92836152 | HEPACAM2.GENEBODY | R | | |
| cg01664065 | chr13: 102249040 | ITGBL1.GENEBODY | R | | |
| cg02097532 | chr3: 55673955 | ERC2.UTR3 | F | | |
| cg02105326 | chr2: 239149142 | HES6.TSS1500 | R | chr2: 239147592-239149900 | Island |
| cg02124724 | chr21: 46875142 | COL18A1.TSS1500 | F | chr21: 46876168-46876752 | N_Shore |
| cg02515354 | chr7: 147256123 | MIR548I4.TSS1500 | F | | |
| cg02638523 | chr2: 236548728 | AGAP1.GENEBODY | R | | |
| cg02754084 | chr12: 72338080 | TPH2.GENEBODY | F | | |
| cg04657588 | chr17: 72427543 | GPRC5C.TSS200 | F | chr17: 72426783-72428977 | Island |
| cg05301383 | chr2: 171569144 | LOC440925.GENEBODY | F | chr2: 171569877-171573904 | N_Shore |
| cg06201680 | chr7: 151078646 | WDR86.UTR3 | R | chr7: 151078462-151079536 | Island |
| cg07449967 | chr8: 56987020 | SNORD54.TSS1500 | R | chr8: 56986556-56987308 | Island |
| cg07471157 | chr12: 49373263 | WNT1.GENEBODY | R | chr12: 49371690-49375550 | Island |
| cg08580631 | chr2: 170220388 | LRP2.TSS1500 | R | chr2: 170220772-170221114 | N_Shore |
| cg09114581 | chr6: 32077103 | TNXB.UTR5 | R | | |
| cg10289744 | chr3: 271185 | CHL1.UTR5 | R | | |
| cg10661054 | chr2: 128422179 | LIMS2.GENEBODY | R | chr2: 128421719-128422182 | Island |
| cg10810657 | chr5: 66203228 | MAST4.GENEBODY | F | | |
| cg10970409 | chr3: 42543566 | VIPR1.TSS1500 | F | chr3: 42542820-42544524 | Island |
| cg11316386 | chr1: 247275808 | C1orf229.TSS200 | R | chr1: 247274585-247275757 | S_Shore |
| cg11642377 | chr7: 8480779 | NXPH1.GENEBODY | R | chr7: 8481974-8482762 | N_Shore |

TABLE 6
Methylation markers for SCLC subtype classification using liquid biopsy

| Methylation | Position | Associated | | | | Relation to |
|---|---|---|---|---|---|---|
| Site | (GRCh 37) | Subgroup | Gene | Strand | CpG Island Name | CpG Island |
| cg00799539 | chr12:103353183 | SCLC-A | ASCL1.UTR3 | R | chr12:103351579-103352695 | S_Shore |
| cg04610718 | chr12:103353362 | SCLC-A | ASCL1.UTR3 | F | chr12:103351579-103352695 | S_Shore |
| cg10672201 | chr12:103353320 | SCLC-A | ASCL1.UTR3 | F | chr12:103351579-103352695 | S_Shore |
| cg17277939 | chr12:70093008 | SCLC-A | BEST3.EXON1 | F | | |
| cg11201256 | chr1:184403226 | SCLC-A | C1orf21.UTR5 | R | | |
| cg07639982 | chr10:12555079 | SCLC-A | CAMK1D.GENEBODY | F | | |
| cg01566028 | chr9:123341267 | SCLC-A | CDK5RAP2.GENEBODY | R | chr9:123342126-123342513 | N_Shore |
| cg06043710 | chr10:560371 | SCLC-A | DIP2C.GENEBODY | F | | |
| cg01154505 | chr2:112940409 | SCLC-A | FBLN7.GENEBODY | F | chr2:112939287-112939563 | S_Shore |
| cg09643186 | chr14:65409452 | SCLC-A | GPX2.EXON1 | F | | |
| cg03817675 | chr7:139299053 | SCLC-A | HIPK2.GENEBODY | R | | |
| cg00090674 | chr22:50446678 | SCLC-A | IL17REL.UTR5 | F | chr22:50448567-50449521 | N_Shore |
| cg02639667 | chr5:154092321 | SCLC-A | LARP1.TSS200 | F | | |
| cg22053861 | chr11:65318683 | SCLC-A | LTBP3.GENEBODY | R | chr11:65321225-65321823 | N_Shelf |
| cg14338051 | chr20:35924146 | SCLC-A | MANBAL.UTR5 | F | | |
| cg00794178 | chr3:182897283 | SCLC-A | MCF2L2.GENEBODY | F | chr3:182896920-182897381 | Island |
| cg16860004 | chr8:103137229 | SCLC-A | NCALD.TSS1500 | F | chr8:103135914-103136775 | S_Shore |
| cg04317756 | chr22:38085445 | SCLC-A | NOL12.GENEBODY | R | chr22:38082302-38082796 | S_Shelf |
| cg01942646 | chr1:27240694 | SCLC-A | NR0B2.TSS200 | R | | |
| cg11249931 | chr18:21796096 | SCLC-A | OSBPL1A.GENEBODY | R | | |
| cg08279731 | chr1:144988231 | SCLC-A | PDE4DIP.GENEBODY | F | | |
| cg05161074 | chr15:42289885 | SCLC-A | PLA2G4E.GENEBODY | F | | |
| cg00773370 | chr12:6483772 | SCLC-A | SCNN1A.GENEBODY | R | | |
| cg07444408 | chr4:141218466 | SCLC-A | SCOC.UTR5 | F | | |
| cg08758185 | chr5:150849936 | SCLC-A | SLC36A1.GENEBODY | F | | |
| cg27119612 | chr15:57491001 | SCLC-A | TCF12.GENEBODY | R | | |
| cg22904437 | chr8:81064304 | SCLC-A | TPD52.GENEBODY | R | | |
| cg04506569 | chr2:234837977 | SCLC-A | TRPM8.GENEBODY | F | | |
| cg04877165 | chr2:174096031 | SCLC-A | ZAK.GENEBODY | R | | |
| cg01610165 | chr5:124071029 | SCLC-A | ZNF608.GENEBODY | R | | |
| cg09248054 | chr1:969257 | SCLC-I | AGRN.GENEBODY | F | chr1:967966-970238 | Island |
| cg02379560 | chr4:40859280 | SCLC-I | APBB2.GENEBODY | F | chr4:40858884-40859162 | S_Shore |
| cg04917391 | chr4:40859259 | SCLC-I | APBB2.GENEBODY | F | chr4:40858884-40859162 | S_Shore |
| cg05020685 | chr4:40858965 | SCLC-I | APBB2.GENEBODY | F | chr4:40858884-40859162 | Island |
| cg06485940 | chr4:40859293 | SCLC-I | APBB2.GENEBODY | F | chr4:40858884-40859162 | S_Shore |
| cg16364495 | chr4:40859251 | SCLC-I | APBB2.GENEBODY | F | chr4:40858884-40859162 | S_Shore |
| cg05857126 | chr16:52108007 | SCLC-I | C16orf97.TSS200 | F | | |
| cg13780782 | chr16:52107988 | SCLC-I | C16orf97.TSS200 | F | | |
| cg02008691 | chr16:52589450 | SCLC-I | CASC16.GENEBODY | F | | |
| cg24664798 | chr16:52640476 | SCLC-I | CASC16.GENEBODY | R | | |
| cg17265693 | chr17:7166490 | SCLC-I | CLDN7.TSS1500 | F | chr17:7164285-7166245 | S_Shore |
| cg06508056 | chr7:146594213 | SCLC-I | CNTNAP2.GENEBODY | R | | |
| cg15932065 | chr7:146470241 | SCLC-I | CNTNAP2.GENEBODY | F | | |
| cg16405211 | chr7:146897691 | SCLC-I | CNTNAP2.GENEBODY | R | | |
| cg18804920 | chr7:146572638 | SCLC-I | CNTNAP2.GENEBODY | R | | |
| cg23889772 | chr19:10077324 | SCLC-I | COL5A3.GENEBODY | F | chr19:10077031-10077444 | Island |
| cg07270851 | chr11:65779332 | SCLC-I | CST6.TSS200 | F | chr11:65779311-65779767 | Island |
| cg24238564 | chr11:65779343 | SCLC-I | CST6.TSS200 | F | chr11:65779311-65779767 | Island |
| cg03850035 | chr2:222367110 | SCLC-I | EPHA4.GENEBODY | R | | |
| cg02659920 | chr11:726335 | SCLC-I | EPS8L2.GENEBODY | F | chr11:725596-726870 | Island |
| cg05651265 | chr11:726367 | SCLC-I | EPS8L2.GENEBODY | R | chr11:725596-726870 | Island |
| cg10976861 | chr2:239149937 | SCLC-I | HES6.TSS1500 | F | chr2:239147592-239149900 | S_Shore |
| cg24127874 | chr2:239149859 | SCLC-I | HES6.TSS1500 | F | chr2:239147592-239149900 | Island |
| cg02365303 | chr5:147052498 | SCLC-I | JAKMIP2.UTR5 | F | | |
| cg11799006 | chr5:147101975 | SCLC-I | JAKMIP2.UTR5 | R | | |
| cg00336977 | chr8:73826000 | SCLC-I | KCNB2.GENEBODY | R | | |
| cg02125259 | chr8:73559340 | SCLC-I | KCNB2.GENEBODY | R | | |
| cg09433131 | chr8:73480179 | SCLC-I | KCNB2.GENEBODY | F | | |
| cg14865862 | chr8:73632304 | SCLC-I | KCNB2.GENEBODY | R | | |
| cg20136513 | chr8:73711260 | SCLC-I | KCNB2.GENEBODY | F | | |
| cg00378510 | chr19:2291020 | SCLC-I | LINGO3.GENEBODY | F | chr19:2289895-2291904 | Island |
| cg06473097 | chr8:73643063 | SCLC-I | LOC101926908.TSS1500 | R | | |
| cg23844705 | chr1:164573835 | SCLC-I | PBX1.GENEBODY | R | | |
| cg19741167 | chr2:86263270 | SCLC-I | POLR1A.GENEBODY | R | | |
| cg16769791 | chr8:85178030 | SCLC-I | RALYL.UTR5 | R | | |
| cg00448761 | chr19:6066102 | SCLC-I | RFX2.UTR5 | R | | |
| cg04303901 | chr12:97886312 | SCLC-I | RMST.GENEBODY | F | | |
| cg13077545 | chr17:41832873 | SCLC-I | SOST.GENEBODY | R | chr17:41832717-41833195 | Island |
| cg15559684 | chr3:63428234 | SCLC-I | SYNPR.GENEBODY | R | | |
| cg07035875 | chr16:52557205 | SCLC-I | TOX3.UTR5 | R | | |
| cg19115272 | chr6:30139538 | SCLC-I | TRIM15.GENEBODY | F | chr6:30139718-30140263 | N_Shore |
| cg06093379 | chr17:44896080 | SCLC-I | WNT3.UTR5 | R | chr17:44895800-44897025 | Island |
| cg19154027 | chr11:101983027 | SCLC-I | YAP1.GENEBODY | F | chr11:101980632-101982330 | S_Shore |
| cg22073838 | chr11:101982817 | SCLC-I | YAP1.TSS1500 | F | chr11:101980632-101982330 | S_Shore |
| cg20505457 | chr14:69388953 | SCLC-N | ACTN1.GENEBODY | F | | |
| cg04220881 | chr7:152524814 | SCLC-N | ACTR3B.GENEBODY | F | | |
| cg14755690 | chr7:128868265 | SCLC-N | AHCYL2.GENEBODY | R | chr7:128864320-128865655 | S_Shelf |
| cg19798702 | chr3:197247751 | SCLC-N | BDH1.GENEBODY | R | | |
| cg03920522 | chr7:94047477 | SCLC-N | COL1A2.GENEBODY | R | | |
| cg12187160 | chr1:9933026 | SCLC-N | CTNNBIP1.UTR5 | R | | |
| cg02531277 | chr7:101558597 | SCLC-N | CUX1.GENEBODY | R | chr7:101559402-101559625 | N_Shore |
| cg18780243 | chr13:32487268 | SCLC-N | EEF1DP3.GENEBODY | R | | |
| cg19028997 | chr1:205603040 | SCLC-N | ELK4.TSS1500 | R | chr1:205600493-205601432 | S_Shore |
| cg02314596 | chr14:89852725 | SCLC-N | FOXN3.GENEBODY | R | | |
| cg16498113 | chr12:42518821 | SCLC-N | GXYLT1.GENEBODY | F | | |
| cg19513004 | chr6:34206683 | SCLC-N | HMGA1.UTR5 | F | chr6:34202567-34206193 | S_Shore |
| cg17401435 | chr11:63887426 | SCLC-N | MACROD1.GENEBODY | F | chr11:63887355-63887600 | Island |
| cg25043538 | chr11:63887437 | SCLC-N | MACROD1.GENEBODY | R | chr11:63887355-63887600 | Island |
| cg00648184 | chr17:60828157 | SCLC-N | MARCH10.GENEBODY | R | | |
| cg10414350 | chr8:10210282 | SCLC-N | MSRA.GENEBODY | R | | |
| cg09309024 | chr16:29837267 | SCLC-N | MVP.UTR5 | F | | |
| cg06440275 | chr8:2005701 | SCLC-N | MYOM2.GENEBODY | R | chr8:2003809-2004059 | S_Shore |
| cg01431993 | chr2:182542864 | SCLC-N | NEUROD1.GENEBODY | R | | |
| cg11528849 | chr2:182542901 | SCLC-N | NEUROD1.GENEBODY | R | | |
| cg16640855 | chr2:182543233 | SCLC-N | NEUROD1.GENEBODY | R | | |
| cg00369376 | chr2:182542510 | SCLC-N | NEUROD1.UTR3 | F | | |
| cg02836529 | chr2:182544413 | SCLC-N | NEUROD1.UTR5 | R | chr2:182547873-182549177 | N_Shelf |
| cg20806345 | chr2:182543706 | SCLC-N | NEUROD1.UTR5 | F | | |
| cg22976218 | chr2:182544321 | SCLC-N | NEUROD1.UTR5 | R | chr2:182547873-182549177 | N_Shelf |
| cg13538006 | chr9:95897313 | SCLC-N | NINJ1.TSS1500 | R | chr9:95896007-95897016 | S_Shore |
| cg11887270 | chr3:52503232 | SCLC-N | NISCH.GENEBODY | F | | |
| cg23731742 | chr1:879958 | SCLC-N | NOC2L.UTR3 | R | chr1:875730-878363 | S_Shore |
| cg02622825 | chr20:34474890 | SCLC-N | PHF20.GENEBODY | F | | |
| cg00284708 | chr8:43147581 | SCLC-N | POTEA.TSS200 | F | | |
| cg05234415 | chr19:38743744 | SCLC-N | PPP1R14A.GENEBODY | R | chr19:38746638-38747379 | N_Shelf |
| cg24657047 | chr15:77317370 | SCLC-N | PSTPIP1.GENEBODY | R | chr15:77320150-77320456 | N_Shelf |
| cg23994112 | chr17:75373457 | SCLC-N | SEPT9.UTR5 | R | chr17:75368688-75370506 | S_Shelf |
| cg08399017 | chr15:65934564 | SCLC-N | SLC24A1.TSS1500 | R | | |
| cg14647703 | chr15:65934619 | SCLC-N | SLC24A1.TSS1500 | R | | |
| cg13969327 | chr7:127526253 | SCLC-N | SND1.GENEBODY | F | | |
| cg05214130 | chr12:123945990 | SCLC-N | SNRNP35.GENEBODY | F | chr12:123942639-123943001 | S_Shelf |
| cg08573199 | chr1:54739682 | SCLC-N | SSBP3.GENEBODY | R | | |
| cg09621610 | chr17:28074897 | SCLC-N | SSH2.GENEBODY | F | | |
| cg13417891 | chr17:35836996 | SCLC-N | TADA2A.GENEBODY | F | | |
| cg02902261 | chr7:72991150 | SCLC-N | TBL2.GENEBODY | R | chr7:72992434-72993397 | N_Shore |
| cg00678971 | chr20:42667505 | SCLC-N | TOX2.GENEBODY | F | | |
| cg02243624 | chr17:66443040 | SCLC-N | WIPI1.GENEBODY | R | | |
| cg13808641 | chr9:96006533 | SCLC-N | WNK2.GENEBODY | R | chr9:96009844-96010101 | N_Shelf |
| cg15076368 | chr3:39224943 | SCLC-N | XIRP1.UTR3 | R | chr3:39222280-39222670 | S_Shelf |
| cg11964216 | chr8:144521570 | SCLC-N | ZC3H3.GENEBODY | R | chr8:144521358-144521608 | Island |
| cg27232389 | chr12:125555734 | SCLC-P | AACS.GENEBODY | R | | |
| cg03020852 | chr7:36446097 | SCLC-P | ANLN.GENEBODY | F | | |
| cg11691710 | chr7:36445998 | SCLC-P | ANLN.GENEBODY | R | | |
| cg14197123 | chr7:36446141 | SCLC-P | ANLN.GENEBODY | R | | |
| cg17885507 | chr7:36445877 | SCLC-P | ANLN.GENEBODY | F | | |
| cg03517570 | chr5:150490911 | SCLC-P | ANXA6.GENEBODY | R | | |
| cg13297671 | chr12:46169924 | SCLC-P | ARID2.GENEBODY | F | | |
| cg18795320 | chr12:14557493 | SCLC-P | ATF7IP.UTR5 | F | | |
| cg03075214 | chr11:64867914 | SCLC-P | C11orf2.GENEBODY | R | chr11:64863698-64863980 | S_Shelf |
| cg06832246 | chr11:93224210 | SCLC-P | C11orf75.UTR5 | R | | |
| cg05683632 | chr19:41253983 | SCLC-P | C19orf54.GENEBODY | F | chr19:41256317-41257234 | N_Shelf |
| cg00704369 | chr7:81996559 | SCLC-P | CACNA2D1.GENEBODY | R | | |
| cg22171098 | chr11:10421566 | SCLC-P | CAND1.11.GENEBODY | F | | |
| cg09934399 | chr19:13858064 | SCLC-P | CCDC130.TSS1500 | R | chr19:13858446-13858904 | N_Shore |
| cg02817764 | chr12:97104034 | SCLC-P | CFAP54.GENEBODY | F | | |
| cg24677222 | chr3:140102647 | SCLC-P | CLSTN2.GENEBODY | F | | |
| cg16770048 | chr22:19201167 | SCLC-P | CLTCL1.GENEBODY | R | | |
| cg14701925 | chr3:128980811 | SCLC-P | COPG.GENEBODY | F | | |
| cg04182076 | chr1:36937561 | SCLC-P | CSF3R.GENEBODY | R | | |
| cg18969798 | chr4:151157003 | SCLC-P | DCLK2.GENEBODY | R | | |
| cg03803789 | chr11:111854003 | SCLC-P | DIXDC1.GENEBODY | R | | |
| cg13240089 | chr3:196902029 | SCLC-P | DLG1.GENEBODY | R | | |
| cg17758792 | chr21:37620038 | SCLC-P | DOPEY2.GENEBODY | F | chr21:37617467-37617872 | S_Shelf |
| cg06923861 | chr11:117355376 | SCLC-P | DSCAML1.GENEBODY | F | chr11:117352691-117352961 | S_Shelf |
| cg07956003 | chr7:95433460 | SCLC-P | DYNC1I1.UTR5 | F | | |
| cg08275278 | chr7:95433492 | SCLC-P | DYNC1I1.UTR5 | F | | |
| cg04983681 | chr2:62952311 | SCLC-P | EHBP1.GENEBODY | R | | |
| cg17728697 | chr4:153434302 | SCLC-P | FBXW7.UTR5 | F | | |
| cg09472222 | chr1:59980198 | SCLC-P | FGGY.TSS1500 | R | | |
| cg02220284 | chr3:71013485 | SCLC-P | FOXP1.GENEBODY | F | | |
| cg05366160 | chr6:33039481 | SCLC-P | HLA-DPA1.GENEBODY | F | | |
| cg13705753 | chr6:33091912 | SCLC-P | HLA-DPB2.GENEBODY | R | | |
| cg06839854 | chr2:10536115 | SCLC-P | HPCAL1.UTR5 | R | | |
| cg02675973 | chr8:73798545 | SCLC-P | KCNB2.GENEBODY | R | | |
| cg05395645 | chr3:197413859 | SCLC-P | KIAA0226.GENEBODY | F | | |
| cg25601286 | chr2:169408033 | SCLC-P | LASS6.GENEBODY | R | | |
| cg11701604 | chr11:36095771 | SCLC-P | LDLRAD3.GENEBODY | F | | |
| cg07825782 | chr1:117021479 | SCLC-P | LOC101929023.TSS200 | R | | |
| cg00600617 | chr6:107221887 | SCLC-P | LOC553137.GENEBODY | F | | |
| cg16054907 | chr22:50706065 | SCLC-P | MAPK11.GENEBODY | R | chr22:50705406-50706553 | Island |
| cg01489441 | chr16:1764111 | SCLC-P | MAPK8IP3.GENEBODY | R | | |
| cg15564226 | chr16:1764329 | SCLC-P | MAPK8IP3.GENEBODY | R | | |
| cg10026427 | chr1:156461666 | SCLC-P | MEF2D.TSS1500 | F | | |
| cg01149239 | chr3:196746085 | SCLC-P | MFI2.UTR3 | F | | |
| cg04479757 | chr22:26951319 | SCLC-P | MIR548J.TSS200 | R | | |
| cg11019743 | chr6:168267395 | SCLC-P | MLLT4.GENEBODY | F | | |
| cg13207778 | chrX:149903801 | SCLC-P | MTMR1.GENEBODY | R | | |
| cg25296465 | chr3:195446826 | SCLC-P | MUC20.TSS1500 | R | | |
| cg03387092 | chr3:123493248 | SCLC-P | MYLK.GENEBODY | R | | |
| cg14175330 | chr9:138949384 | SCLC-P | NACC2.UTR5 | R | | |
| cg05999426 | chr20:46203476 | SCLC-P | NCOA3.UTR5 | F | | |
| cg19011089 | chr20:33307672 | SCLC-P | NCOA6.GENEBODY | R | | |
| cg14313328 | chr1:54301402 | SCLC-P | NDC1.GENEBODY | R | chr1:54303758-54304262 | N_Shelf |
| cg18529415 | chr1:41174259 | SCLC-P | NFYC.TSS1500 | R | | |
| cg04563108 | chr18:56715086 | SCLC-P | OACYLP.GENEBODY | R | | |
| cg25874150 | chr10:34941561 | SCLC-P | PARD3.GENEBODY | R | | |
| cg16318349 | chr1:154917307 | SCLC-P | PBXIP1.UTR3 | R | | |
| cg13081156 | chr5:134280996 | SCLC-P | PCBD2.GENEBODY | F | | |
| cg14088354 | chr1:233275911 | SCLC-P | PCNXL2.GENEBODY | R | | |
| cg03264729 | chr11:72347339 | SCLC-P | PDE2A.GENEBODY | R | | |
| cg01580044 | chr1:114300656 | SCLC-P | PHTF1.GENEBODY | R | chr1:114301514-114302272 | N_Shore |
| cg07313835 | chr1:167342724 | SCLC-P | POU2F1.GENEBODY | F | | |
| cg11597418 | chr11:120165937 | SCLC-P | POU2F3.GENEBODY | F | | |
| cg26855672 | chr11:120113086 | SCLC-P | POU2F3.GENEBODY | R | chr11:120110497-120110719 | S_Shelf |
| cg03356778 | chr11:120110544 | SCLC-P | POU2F3.TSS1500 | F | chr11:120110497-120110719 | Island |
| cg05216984 | chr2:242109132 | SCLC-P | PPP1R7.GENEBODY | R | | |
| cg02593168 | chr2:231931446 | SCLC-P | PSMD1.GENEBODY | F | | |
| cg20314331 | chr2:131129567 | SCLC-P | PTPN18.GENEBODY | R | chr2:131129718-131130511 | N_Shore |
| cg26797073 | chr2:131129811 | SCLC-P | PTPN18.GENEBODY | F | chr2:131129718-131130511 | Island |
| cg15873449 | chr18:12885569 | SCLC-P | PTPN2.TSS1500 | F | chr18:12883285-12884578 | S_Shore |
| cg18983310 | chr5:86603732 | SCLC-P | RASA1.GENEBODY | F | | |
| cg08265811 | chr2:178975718 | SCLC-P | RBM45.TSS1500 | R | chr2:178977078-178977828 | N_Shore |
| cg22340762 | chr1:183823538 | SCLC-P | RGL1.GENEBODY | F | | |
| cg00618183 | chr17:75143600 | SCLC-P | SEC14L1.GENEBODY | R | | |
| cg12452539 | chr3:150325281 | SCLC-P | SELT.GENEBODY | F | chr3:150321002-150321791 | S_Shelf |
| cg15786168 | chr1:151117254 | SCLC-P | SEMA6C.UTR5 | F | chr1:151118049-151119373 | N_Shore |
| cg06669752 | chr5:138534431 | SCLC-P | SIL1.TSS1500 | R | chr5:138533870-138534113 | S_Shore |
| cg24136932 | chr8:22281979 | SCLC-P | SLC39A14.GENEBODY | R | | |
| cg24272324 | chr18:48603733 | SCLC-P | SMAD4.GENEBODY | R | | |
| cg04619304 | chr12:24434537 | SCLC-P | SOX5.UTR5 | R | | |
| cg01772824 | chr17:28041399 | SCLC-P | SSH2.GENEBODY | R | | |
| cg13921012 | chr1:85119175 | SCLC-P | SSX2IP.GENEBODY | F | | |
| cg06542565 | chr9:126000925 | SCLC-P | STRBP.GENEBODY | R | | |
| cg10288437 | chr6:158432894 | SCLC-P | SYNJ2.GENEBODY | F | | |
| cg10609655 | chr20:60615080 | SCLC-P | TAF4.GENEBODY | R | | |
| cg07262682 | chr7:150494758 | SCLC-P | TMEM176B.UTR5 | R | chr7:150496809-150498206 | N_Shelf |
| cg21289124 | chr22:26951249 | SCLC-P | TPST2.UTR5 | R | | |
| cg07325342 | chr16:3728204 | SCLC-P | TRAP1.GENEBODY | F | chr16:3724344-3724849 | S_Shelf |
| cg14211075 | chr9:135818481 | SCLC-P | TSC1.UTR5 | R | chr9:135819526-135820529 | N_Shore |
| cg00719067 | chr13:27687798 | SCLC-P | USP12.GENEBODY | R | | |
| cg05554594 | chr17:80584498 | SCLC-P | WDR45L.GENEBODY | F | | |
| cg08368885 | chr9:129596150 | SCLC-P | ZBTB43.GENEBODY | F | chr9:129595357-129595584 | S_Shore |
| cg21876001 | chr19:11594640 | SCLC-P | ZNF653.GENEBODY | R | chr19:11594372-11594987 | Island |

TABLE 7
Methylation markers for classification of SCLC-A using liquid biopsy

| | | | | | Relation to |
|---|---|---|---|---|---|
| Methylation Site | Position (GRCh 37) | Gene | Strand | CpG Island Name | CpG Island |
| cg00799539 | chr12: 103353183 | ASCL1.UTR3 | R | chr12: 103351579- | S_Shore |
| | | | | 103352695 | |
| cg04610718 | chr12: 103353362 | ASCL1.UTR3 | F | chr12: 103351579- | S_Shore |
| | | | | 103352695 | |
| cg10672201 | chr12: 103353320 | ASCL1.UTR3 | F | chr12: 103351579- | S_Shore |
| | | | | 103352695 | |
| cg17277939 | chr12: 70093008 | BEST3.EXON1 | F | | |
| cg11201256 | chr1: 184403226 | C1orf21.UTR5 | R | | |
| cg07639982 | chr10: 12555079 | CAMK1D.GENEBODY | F | | |
| cg01566028 | chr9: 123341267 | CDK5RAP2.GENEBODY | R | chr9: 123342126- | N_Shore |
| | | | | 123342513 | |
| cg06043710 | chr10: 560371 | DIP2C.GENEBODY | F | | |
| cg01154505 | chr2: 112940409 | FBLN7.GENEBODY | F | chr2: 112939287- | S_Shore |
| | | | | 112939563 | |
| cg09643186 | chr14: 65409452 | GPX2.EXON1 | F | | |
| cg03817675 | chr7: 139299053 | HIPK2.GENEBODY | R | | |
| cg00090674 | chr22: 50446678 | IL17REL.UTR5 | F | chr22: 50448567- | N_Shore |
| | | | | 50449521 | |
| cg02639667 | chr5: 154092321 | LARP1.TSS200 | F | | |
| cg22053861 | chr11: 65318683 | LTBP3.GENEBODY | R | chr11: 65321225- | N_Shelf |
| | | | | 65321823 | |
| cg14338051 | chr20: 35924146 | MANBAL.UTR5 | F | | |
| cg00794178 | chr3: 182897283 | MCF2L2.GENEBODY | F | chr3: 182896920- | Island |
| | | | | 182897381 | |
| cg16860004 | chr8: 103137229 | NCALD.TSS1500 | F | chr8: 103135914- | S_Shore |
| | | | | 103136775 | |
| cg04317756 | chr22: 38085445 | NOL12.GENEBODY | R | chr22: 38082302- | S_Shelf |
| | | | | 38082796 | |
| cg01942646 | chr1: 27240694 | NROB2.TSS200 | R | | |
| cg11249931 | chr18: 21796096 | OSBPL1A.GENEBODY | R | | |
| cg08279731 | chr1: 144988231 | PDE4DIP.GENEBODY | F | | |
| cg05161074 | chr15: 42289885 | PLA2G4E.GENEBODY | F | | |
| cg00773370 | chr12: 6483772 | SCNN1A.GENEBODY | R | | |
| cg07444408 | chr4: 141218466 | SCOC.UTR5 | F | | |
| cg08758185 | chr5: 150849936 | SLC36A1.GENEBODY | F | | |
| cg27119612 | chr15: 57491001 | TCF12.GENEBODY | R | | |
| cg22904437 | chr8: 81064304 | TPD52.GENEBODY | R | | |
| cg04506569 | chr2: 234837977 | TRPM8.GENEBODY | F | | |
| cg04877165 | chr2: 174096031 | ZAK.GENEBODY | R | | |
| cg01610165 | chr5: 124071029 | ZNF608.GENEBODY | R | | |

TABLE 8
Methylation markers for classification of SCLC-N using liquid biopsy

| | | | | | Relation to |
|---|---|---|---|---|---|
| Methylation Site | Position (GRCh 37) | Gene | Strand | CpG Island Name | CpG Island |
| cg20505457 | chr14: 69388953 | ACTN1.GENEBODY | F | | |
| cg04220881 | chr7: 152524814 | ACTR3B.GENEBODY | F | | |
| cg14755690 | chr7: 128868265 | AHCYL2.GENEBODY | R | chr7: 128864320- | S_Shelf |
| | | | | 128865655 | |
| cg19798702 | chr3: 197247751 | BDH1.GENEBODY | R | | |
| cg03920522 | chr7: 94047477 | COL1A2.GENEBODY | R | | |
| cg12187160 | chr1: 9933026 | CTNNBIP1.UTR5 | R | | |
| cg02531277 | chr7: 101558597 | CUX1.GENEBODY | R | chr7: 101559402- | N_Shore |
| | | | | 101559625 | |
| cg18780243 | chr13: 32487268 | EEF1DP3.GENEBODY | R | | |
| cg19028997 | chr1: 205603040 | ELK4.TSS1500 | R | chr1: 205600493- | S_Shore |
| | | | | 205601432 | |
| cg02314596 | chr14: 89852725 | FOXN3.GENEBODY | R | | |
| cg16498113 | chr12: 42518821 | GXYLT1.GENEBODY | F | | |
| cg19513004 | chr6: 34206683 | HMGA1.UTR5 | F | chr6: 34202567- | S_Shore |
| | | | | 34206193 | |
| cg17401435 | chr11: 63887426 | MACROD1.GENEBODY | F | chr11: 63887355- | Island |
| | | | | 63887600 | |
| cg25043538 | chr11: 63887437 | MACROD1.GENEBODY | R | chr11: 63887355- | Island |
| | | | | 63887600 | |
| cg00648184 | chr17: 60828157 | MARCH10.GENEBODY | R | | |
| cg10414350 | chr8: 10210282 | MSRA.GENEBODY | R | | |
| cg09309024 | chr16: 29837267 | MVP.UTR5 | F | | |
| cg06440275 | chr8: 2005701 | MYOM2.GENEBODY | R | chr8: 2003809- | S_Shore |
| | | | | 2004059 | |
| cg01431993 | chr2: 182542864 | NEUROD1.GENEBODY | R | | |
| cg11528849 | chr2: 182542901 | NEUROD1.GENEBODY | R | | |
| cg16640855 | chr2: 182543233 | NEUROD1.GENEBODY | R | | |
| cg00369376 | chr2: 182542510 | NEUROD1.UTR3 | F | | |
| cg02836529 | chr2: 182544413 | NEUROD1.UTR5 | R | chr2: 182547873- | N_Shelf |
| | | | | 182549177 | |
| cg20806345 | chr2: 182543706 | NEUROD1.UTR5 | F | | |
| cg22976218 | chr2: 182544321 | NEUROD1.UTR5 | R | chr2: 182547873- | N_Shelf |
| | | | | 182549177 | |
| cg13538006 | chr9: 95897313 | NINJ1.TSS1500 | R | chr9: 95896007- | S_Shore |
| | | | | 95897016 | |
| cg11887270 | chr3: 52503232 | NISCH.GENEBODY | F | | |
| cg23731742 | chr1: 879958 | NOC2L.UTR3 | R | chr1: 875730- | S_Shore |
| | | | | 878363 | |
| cg02622825 | chr20: 34474890 | PHF20.GENEBODY | F | | |
| cg00284708 | chr8: 43147581 | POTEA.TSS200 | F | | |
| cg05234415 | chr19: 38743744 | PPP1R14A.GENEBODY | R | chr19: 38746638- | N_Shelf |
| | | | | 38747379 | |
| cg24657047 | chr15: 77317370 | PSTPIP1.GENEBODY | R | chr15: 77320150- | N_Shelf |
| | | | | 77320456 | |
| cg23994112 | chr17: 75373457 | SEPT9.UTR5 | R | chr17: 75368688- | S_Shelf |
| | | | | 75370506 | |
| cg08399017 | chr15: 65934564 | SLC24A1.TSS1500 | R | | |
| cg14647703 | chr15: 65934619 | SLC24A1.TSS1500 | R | | |
| cg13969327 | chr7: 127526253 | SND1.GENEBODY | F | | |
| cg05214130 | chr12: 123945990 | SNRNP35.GENEBODY | F | chr12: 123942639- | S_Shelf |
| | | | | 123943001 | |
| cg08573199 | chr1: 54739682 | SSBP3.GENEBODY | R | | |
| cg09621610 | chr17: 28074897 | SSH2.GENEBODY | F | | |
| cg13417891 | chr17: 35836996 | TADA2A.GENEBODY | F | | |
| cg02902261 | chr7: 72991150 | TBL2.GENEBODY | R | chr7: 72992434- | N_Shore |
| | | | | 72993397 | |
| cg00678971 | chr20: 42667505 | TOX2.GENEBODY | F | | |
| cg02243624 | chr17: 66443040 | WIPI1.GENEBODY | R | | |
| cg13808641 | chr9: 96006533 | WNK2.GENEBODY | R | chr9: 96009844- | N_Shelf |
| | | | | 96010101 | |
| cg15076368 | chr3: 39224943 | XIRP1.UTR3 | R | chr3: 39222280- | S_Shelf |
| | | | | 39222670 | |
| cg11964216 | chr8: 144521570 | ZC3H3.GENEBODY | R | chr8: 144521358- | Island |
| | | | | 144521608 | |

TABLE 9
Methylation markers for classification of SCLC-P using liquid biopsy

| | | | | | Relation to |
|---|---|---|---|---|---|
| Methylation Site | Position (GRCh 37) | Gene | Strand | CpG Island Name | CpG Island |
| cg27232389 | chr12: 125555734 | AACS.GENEBODY | R | | |
| cg03020852 | chr7: 36446097 | ANLN.GENEBODY | F | | |
| cg11691710 | chr7: 36445998 | ANLN.GENEBODY | R | | |
| cg14197123 | chr7: 36446141 | ANLN.GENEBODY | R | | |
| cg17885507 | chr7: 36445877 | ANLN.GENEBODY | F | | |
| cg03517570 | chr5: 150490911 | ANXA6.GENEBODY | R | | |
| cg13297671 | chr12: 46169924 | ARID2.GENEBODY | F | | |
| cg18795320 | chr12: 14557493 | ATF7IP.UTR5 | F | | |
| cg03075214 | chr11: 64867914 | C11orf2.GENEBODY | R | chr11: 64863698- | S_Shelf |
| | | | | 64863980 | |
| cg06832246 | chr11: 93224210 | C11orf75.UTR5 | R | | |
| cg05683632 | chr19: 41253983 | C19orf54.GENEBODY | F | chr19: 41256317- | N_Shelf |
| | | | | 41257234 | |
| cg00704369 | chr7: 81996559 | CACNA2D1.GENEBODY | R | | |
| cg22171098 | chr11: 10421566 | CAND1.11.GENEBODY | F | | |
| cg09934399 | chr19: 13858064 | CCDC130.TSS1500 | R | chr19: 13858446- | N_Shore |
| | | | | 13858904 | |
| cg02817764 | chr12: 97104034 | CFAP54.GENEBODY | F | | |
| cg24677222 | chr3: 140102647 | CLSTN2.GENEBODY | F | | |
| cg16770048 | chr22: 19201167 | CLTCL1.GENEBODY | R | | |
| cg14701925 | chr3: 128980811 | COPG.GENEBODY | F | | |
| cg04182076 | chr1: 36937561 | CSF3R.GENEBODY | R | | |
| cg18969798 | chr4: 151157003 | DCLK2.GENEBODY | R | | |
| cg03803789 | chr11: 111854003 | DIXDC1.GENEBODY | R | | |
| cg13240089 | chr3: 196902029 | DLG1.GENEBODY | R | | |
| cg17758792 | chr21: 37620038 | DOPEY2.GENEBODY | F | chr21: 37617467- | S Shelf |
| | | | | 37617872 | |
| cg06923861 | chr11: 117355376 | DSCAML1.GENEBODY | F | chr11: 117352691- | S_Shelf |
| | | | | 117352961 | |
| cg07956003 | chr7: 95433460 | DYNC111.UTR5 | F | | |
| cg08275278 | chr7: 95433492 | DYNC111.UTR5 | F | | |
| cg04983681 | chr2: 62952311 | EHBP1.GENEBODY | R | | |
| cg17728697 | chr4: 153434302 | FBXW7.UTR5 | F | | |
| cg09472222 | chr1: 59980198 | FGGY.TSS1500 | R | | |
| cg02220284 | chr3: 71013485 | FOXP1.GENEBODY | F | | |
| cg05366160 | chr6: 33039481 | HLA-DPA1.GENEBODY | F | | |
| cg13705753 | chr6: 33091912 | HLA-DPB2.GENEBODY | R | | |
| cg06839854 | chr2: 10536115 | HPCAL1.UTR5 | R | | |
| cg02675973 | chr8: 73798545 | KCNB2.GENEBODY | R | | |
| cg05395645 | chr3: 197413859 | KIAA0226.GENEBODY | F | | |
| cg25601286 | chr2: 169408033 | LASS6.GENEBODY | R | | |
| cg11701604 | chr11: 36095771 | LDLRAD3.GENEBODY | F | | |
| cg07825782 | chr1: 117021479 | LOC101929023.TSS200 | R | | |
| cg00600617 | chr6: 107221887 | LOC553137.GENEBODY | F | | |
| cg16054907 | chr22: 50706065 | MAPK11.GENEBODY | R | chr22: 50705406- | Island |
| | | | | 50706553 | |
| cg01489441 | chr16: 1764111 | MAPK8IP3.GENEBODY | R | | |
| cg15564226 | chr16: 1764329 | MAPK8IP3.GENEBODY | R | | |
| cg10026427 | chr1: 156461666 | MEF2D.TSS1500 | F | | |
| cg01149239 | chr3: 196746085 | MFI2.UTR3 | F | | |
| cg04479757 | chr22: 26951319 | MIR548J.TSS200 | R | | |
| cg11019743 | chr6: 168267395 | MLLT4.GENEBODY | F | | |
| cg13207778 | chrX: 149903801 | MTMR1.GENEBODY | R | | |
| cg25296465 | chr3: 195446826 | MUC20.TSS1500 | R | | |
| cg03387092 | chr3: 123493248 | MYLK.GENEBODY | R | | |
| cg14175330 | chr9: 138949384 | NACC2.UTR5 | R | | |
| cg05999426 | chr20: 46203476 | NCOA3.UTR5 | F | | |
| cg19011089 | chr20: 33307672 | NCOA6.GENEBODY | R | | |
| cg14313328 | chr1: 54301402 | NDC1.GENEBODY | R | chr1: 54303758- | N Shelf |
| | | | | 54304262 | |
| cg18529415 | chr1: 41174259 | NFYC.TSS1500 | R | | |
| cg04563108 | chr18: 56715086 | OACYLP.GENEBODY | R | | |
| cg25874150 | chr10: 34941561 | PARD3.GENEBODY | R | | |
| cg16318349 | chr1: 154917307 | PBXIP1.UTR3 | R | | |
| cg13081156 | chr5: 134280996 | PCBD2.GENEBODY | F | | |
| cg14088354 | chr1: 233275911 | PCNXL2.GENEBODY | R | | |
| cg03264729 | chr11: 72347339 | PDE2A.GENEBODY | R | | |
| cg01580044 | chr1: 114300656 | PHTF1.GENEBODY | R | chr1: 114301514- | N_Shore |
| | | | | 114302272 | |
| cg07313835 | chr1: 167342724 | POU2F1.GENEBODY | F | | |
| cg11597418 | chr11: 120165937 | POU2F3.GENEBODY | F | | |
| cg26855672 | chr11: 120113086 | POU2F3.GENEBODY | R | chr11: 120110497- | S_Shelf |
| | | | | 120110719 | |
| cg03356778 | chr11: 120110544 | POU2F3.TSS1500 | F | chr11: 120110497- | Island |
| | | | | 120110719 | |
| cg05216984 | chr2: 242109132 | PPP1R7.GENEBODY | R | | |
| cg02593168 | chr2: 231931446 | PSMD1.GENEBODY | F | | |
| cg20314331 | chr2: 131129567 | PTPN18.GENEBODY | R | chr2: 131129718- | N_Shore |
| | | | | 131130511 | |
| cg26797073 | chr2: 131129811 | PTPN18.GENEBODY | F | chr2: 131129718- | Island |
| | | | | 131130511 | |
| cg15873449 | chr18: 12885569 | PTPN2.TSS1500 | F | chr18: 12883285- | S_Shore |
| | | | | 12884578 | |
| cg18983310 | chr5: 86603732 | RASA1.GENEBODY | F | | |
| cg08265811 | chr2: 178975718 | RBM45.TSS1500 | R | chr2: 178977078- | N_Shore |
| | | | | 178977828 | |
| cg22340762 | chr1: 183823538 | RGL1.GENEBODY | F | | |
| cg00618183 | chr17: 75143600 | SEC14L1.GENEBODY | R | | |
| cg12452539 | chr3: 150325281 | SELT.GENEBODY | F | chr3: 150321002- | S_Shelf |
| | | | | 150321791 | |
| cg15786168 | chr1: 151117254 | SEMA6C.UTR5 | F | chr1: 151118049- | N_Shore |
| | | | | 151119373 | |
| cg06669752 | chr5: 138534431 | SIL1.TSS1500 | R | chr5: 138533870- | S_Shore |
| | | | | 138534113 | |
| cg24136932 | chr8: 22281979 | SLC39A14.GENEBODY | R | | |
| cg24272324 | chr18: 48603733 | SMAD4.GENEBODY | R | | |
| cg04619304 | chr12: 24434537 | SOX5.UTR5 | R | | |
| cg01772824 | chr17: 28041399 | SSH2.GENEBODY | R | | |
| cg13921012 | chr1: 85119175 | SSX2IP.GENEBODY | F | | |
| cg06542565 | chr9: 126000925 | STRBP.GENEBODY | R | | |
| cg10288437 | chr6: 158432894 | SYNJ2.GENEBODY | F | | |
| cg10609655 | chr20: 60615080 | TAF4.GENEBODY | R | | |
| cg07262682 | chr7: 150494758 | TMEM176B.UTR5 | R | chr7: 150496809- | N_Shelf |
| | | | | 150498206 | |
| cg21289124 | chr22: 26951249 | TPST2.UTR5 | R | | |
| cg07325342 | chr16: 3728204 | TRAP1.GENEBODY | F | chr16: 3724344- | S_Shelf |
| | | | | 3724849 | |
| cg14211075 | chr9: 135818481 | TSC1.UTR5 | R | chr9: 135819526- | N_Shore |
| | | | | 135820529 | |
| cg00719067 | chr13: 27687798 | USP12.GENEBODY | R | | |
| cg05554594 | chr17: 80584498 | WDR45L.GENEBODY | F | | |
| cg08368885 | chr9: 129596150 | ZBTB43.GENEBODY | F | chr9: 129595357- | S_Shore |
| | | | | 129595584 | |
| cg21876001 | chr19: 11594640 | ZNF653.GENEBODY | R | chr19: 11594372- | Island |
| | | | | 11594987 | |

TABLE 10
Methylation markers for classification of SCLC-I using liquid biopsy

| | | | | | Relation to |
|---|---|---|---|---|---|
| Methylation Site | Position (GRCh 37) | Gene | Strand | CpG Island Name | CpG Island |
| cg09248054 | chr1: 969257 | AGRN.GENEBODY | F | chr1: 967966- | Island |
| | | | | 970238 | |
| cg02379560 | chr4: 40859280 | APBB2.GENEBODY | F | chr4: 40858884- | S_Shore |
| | | | | 40859162 | |
| cg04917391 | chr4: 40859259 | APBB2.GENEBODY | F | chr4: 40858884- | S_Shore |
| | | | | 40859162 | |
| cg05020685 | chr4: 40858965 | APBB2.GENEBODY | F | chr4: 40858884- | Island |
| | | | | 40859162 | |
| cg06485940 | chr4: 40859293 | APBB2.GENEBODY | F | chr4: 40858884- | S_Shore |
| | | | | 40859162 | |
| cg16364495 | chr4: 40859251 | APBB2.GENEBODY | F | chr4: 40858884- | S_Shore |
| | | | | 40859162 | |
| cg05857126 | chr16: 52108007 | C16orf97.TSS200 | F | | |
| cg13780782 | chr16: 52107988 | C16orf97.TSS200 | F | | |
| cg02008691 | chr16: 52589450 | CASC16.GENEBODY | F | | |
| cg24664798 | chr16: 52640476 | CASC16.GENEBODY | R | | |
| cg17265693 | chr17: 7166490 | CLDN7.TSS1500 | F | chr17: 7164285- | S_Shore |
| | | | | 7166245 | |
| cg06508056 | chr7: 146594213 | CNTNAP2.GENEBODY | R | | |
| cg15932065 | chr7: 146470241 | CNTNAP2.GENEBODY | F | | |
| cg16405211 | chr7: 146897691 | CNTNAP2.GENEBODY | R | | |
| cg18804920 | chr7: 146572638 | CNTNAP2.GENEBODY | R | | |
| cg23889772 | chr19: 10077324 | COL5A3.GENEBODY | F | chr19: 10077031- | Island |
| | | | | 10077444 | |
| cg07270851 | chr11: 65779332 | CST6.TSS200 | F | chr11: 65779311- | Island |
| | | | | 65779767 | |
| cg24238564 | chr11: 65779343 | CST6.TSS200 | F | chr11: 65779311- | Island |
| | | | | 65779767 | |
| cg03850035 | chr2: 222367110 | EPHA4.GENEBODY | R | | |
| cg02659920 | chr11: 726335 | EPS8L2.GENEBODY | F | chr11: 725596- | Island |
| | | | | 726870 | |
| cg05651265 | chr11: 726367 | EPS8L2.GENEBODY | R | chr11: 725596- | Island |
| | | | | 726870 | |
| cg10976861 | chr2: 239149937 | HES6.TSS1500 | F | chr2: 239147592- | S_Shore |
| | | | | 239149900 | |
| cg24127874 | chr2: 239149859 | HES6.TSS1500 | F | chr2: 239147592- | Island |
| | | | | 239149900 | |
| cg02365303 | chr5: 147052498 | JAKMIP2.UTR5 | F | | |
| cg11799006 | chr5: 147101975 | JAKMIP2.UTR5 | R | | |
| cg00336977 | chr8: 73826000 | KCNB2.GENEBODY | R | | |
| cg02125259 | chr8: 73559340 | KCNB2.GENEBODY | R | | |
| cg09433131 | chr8: 73480179 | KCNB2.GENEBODY | F | | |
| cg14865862 | chr8: 73632304 | KCNB2.GENEBODY | R | | |
| cg20136513 | chr8: 73711260 | KCNB2.GENEBODY | F | | |
| cg00378510 | chr19: 2291020 | LINGO3.GENEBODY | F | chr19: 2289895- | Island |
| | | | | 2291904 | |
| cg06473097 | chr8: 73643063 | LOC101926908.TSS1500 | R | | |
| cg23844705 | chr1: 164573835 | PBX1.GENEBODY | R | | |
| cg19741167 | chr2: 86263270 | POLR1A.GENEBODY | R | | |
| cg16769791 | chr8: 85178030 | RALYL.UTR5 | R | | |
| cg00448761 | chr19: 6066102 | RFX2.UTR5 | R | | |
| cg04303901 | chr12: 97886312 | RMST.GENEBODY | F | | |
| cg13077545 | chr17: 41832873 | SOST.GENEBODY | R | chr17: 41832717- | Island |
| | | | | 41833195 | |
| cg15559684 | chr3: 63428234 | SYNPR.GENEBODY | R | | |
| cg07035875 | chr16: 52557205 | TOX3.UTR5 | R | | |
| cg19115272 | chr6: 30139538 | TRIM15.GENEBODY | F | chr6: 30139718- | N_Shore |
| | | | | 30140263 | |
| cg06093379 | chr17: 44896080 | WNT3.UTR5 | R | chr17: 44895800- | Island |
| | | | | 44897025 | |
| cg19154027 | chr11: 101983027 | YAP1.GENEBODY | F | chr11: 101980632- | S_Shore |
| | | | | 101982330 | |
| cg22073838 | chr11: 101982817 | YAP1.TSS1500 | F | chr11: 101980632- | S_Shore |
| | | | | 101982330 | |

TABLE 11
Top 10 Combinations of two genes for the differentiation of SCLC-A

| Methylation | Position | LogReg | RF | |
|---|---|---|---|---|
| Sites | (GRCh 37) | (Mean AUC)¹ | (MeanAUC)² | Name |
| cg17542751_cg26319503 | chr22: 44568725_chr1: | 1.0000000 | 0.8930833 | PARVG.TSS200_GRIK3.GENEBODY |
| | 37313328 | | | |
| cg08581399_cg00512287 | chr8: 110305656_chr11: | 0.9918611 | NA | NUDCD1.GENEBODY_PHLDB1.GENEBODY |
| | 118512372 | | | |
| cg02290110_cg25353896 | chr19: 461808_chr22: | 0.9917222 | NA | SHC2.TSS1500_PARVG.TSS200 |
| | 44568753 | | | |
| cg13429423_cg26319503 | chr22: 44568775_chr1: | 0.9898889 | 0.9830000 | PARVG.TSS200_GRIK3.GENEBODY |
| | 37313328 | | | |
| cg14503873_cg05930278 | chr9: 117036748_chr3: | 0.9883889 | 0.9866667 | COL27A1.GENEBODY_SH3BP5.GENEBODY |
| | 15362399 | | | |
| cg00512287_cg16777369 | chr11: 118512372_chr10: | 0.9877500 | 0.9844444 | PHLDB1.GENEBODY_TCERG1L.GENEBODY |
| | 132900674 | | | |
| cg17542751_cg26148923 | chr22: 44568725_chr3: | 0.9866944 | NA | PARVG.TSS200_LINC00578.GENEBODY |
| | 177310535 | | | |
| cg14503873_cg25694807 | chr9: 117036748_chr2: | 0.9855833 | 0.9869167 | COL27A1.GENEBODY_RBMS1.GENEBODY |
| | 161329607 | | | |
| cg13703062_cg25433259 | chr18: 32924349_chr6: | 0.9858056 | NA | ZNF24.EXON1_OSTCP1.GENEBODY |
| | 159271784 | | | |
| cg15892280_cg26319503 | chr21: 40180000_chr1: | 0.9853056 | NA | ETS2.UTR5_GRIK3.GENEBODY |
| | 37313328 | | | |

¹Mean area under the curve as determined by the logistic regression model (LogReg).
²Mean area under the curve as determined by the random forrest model (RF).

TABLE 12
Top 10 Combinations of two genes for the differentiation of SCLC-N

| Methylation | Position | LogReg | RF | |
|---|---|---|---|---|
| Sites | (GRCh 37) | (Mean AUC)¹ | (Mean AUC)² | Name |
| cg10468340_cg05202032 | chr3: 58318421_chr17: | 1.0000000 | 0.9963333 | PXK.TSS200_VPS53.GENEBODY |
| | 424205 | | | |
| cg10468340_cg05328630 | chr3: 58318421_chr13: | 1.0000000 | 1.0000000 | PXK.TSS200_CLYBL.GENEBODY |
| | 100506703 | | | |
| cg05328630_cg05212450 | chr13: 100506703_chr7: | 0.9900000 | NA | CLYBL.GENEBODY MAD1L1.GENEBODY |
| | 2149814 | | | |
| cg17589175_cg23045716 | chr11: 64085337_chr4: | 0.9890000 | NA | PRDXS.TSS1500_LOC100130872.GENEBODY |
| | 1201881 | | | |
| cg22557350_cg25870970 | chr10: 126432983_chr20: | 0.9871481 | NA | FAMS3B.TSS200_ATPSA.GENEBODY |
| | 50294927 | | | |
| cg15193475_cg02243624 | chr15: 80998711_chr17: | 0.9870000 | NA | FAM108C1.GENEBODY_WIPI1.GENEBODY |
| | 66443040 | | | |
| cg22976218_cg19028997 | chr2: 182544321_chr1: | 0.8869259 | 0.9814444 | NEUROD1.UTR5_ELK4.TSS1500 |
| | 205603040 | | | |
| cg14647703_cg23516715 | chr15: 65934619_chrX: | 0.9858889 | 0.9833333 | SLC24A1.TSS1500_EDA.GENEBODY |
| | 68890853 | | | |
| cg27088449_cg10414350 | chr22: 21272634_chr8: | 0.9866667 | NA | CRKL.GENEBODY_MSRA.GENEBODY |
| | 10210282 | | | |
| cg01789478_cg08399017 | chr2: 182545485_chr15: | 0.9865556 | NA | NEUROD1.TSS200_SLC24A1.TSS1500 |
| | 65934564 | | | |

¹Mean area under the curve as determined by the logistic regression model (LogReg).
²Mean area under the curve as determined by the random forrest model (RF).

### Example 2—Identification of Specific Markers for SCLC

Using the same methods described in Example 1, 27 markers were identified that are useful for classifying SCLC specifically vs. other types of cancers, and that are sufficiently differentially methylated to be included in a liquid biopsy assay. The list of markers is provided in Table 13. To get better specificities, only lung SCLC, lung Adenocarcinoma and lung Squamous cell carcinoma cell lines were included in the marker selection process.

To make sure that the markers were not only effective for analysis of SCLC cell lines, methylation levels were analyzed using the GSE60644 dataset, comprising 124 lung cancers from various histologies (FIG. 11). Importantly, the markers derived from the cell lines were indeed also capable of uniquely distinguishing SCLC in analysis of tissue samples.

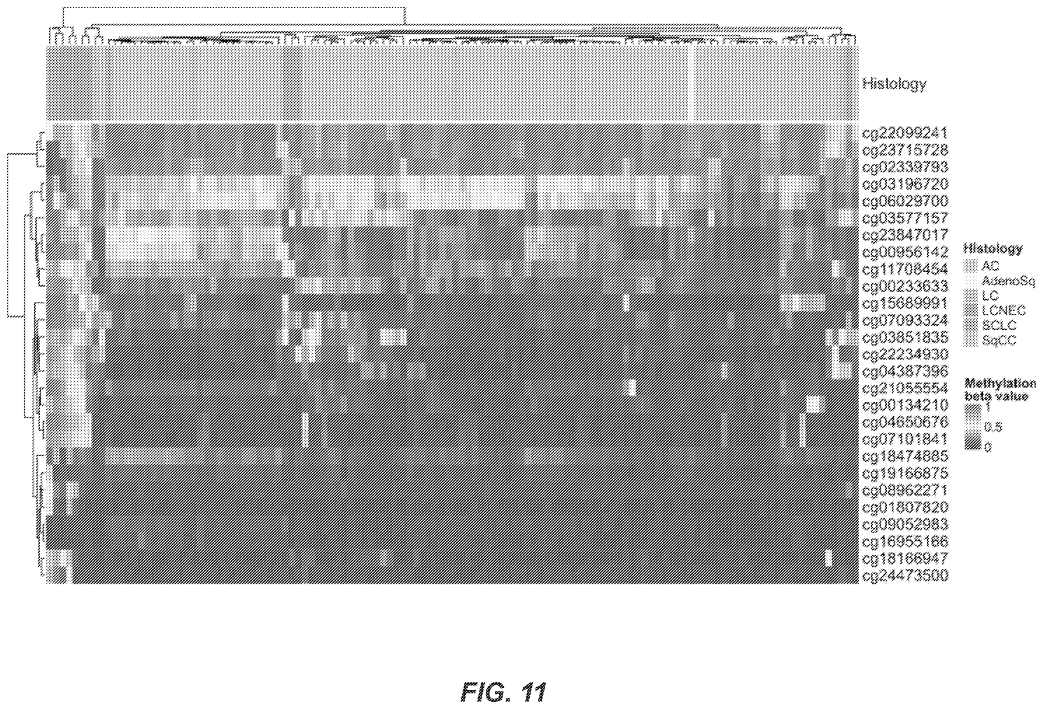

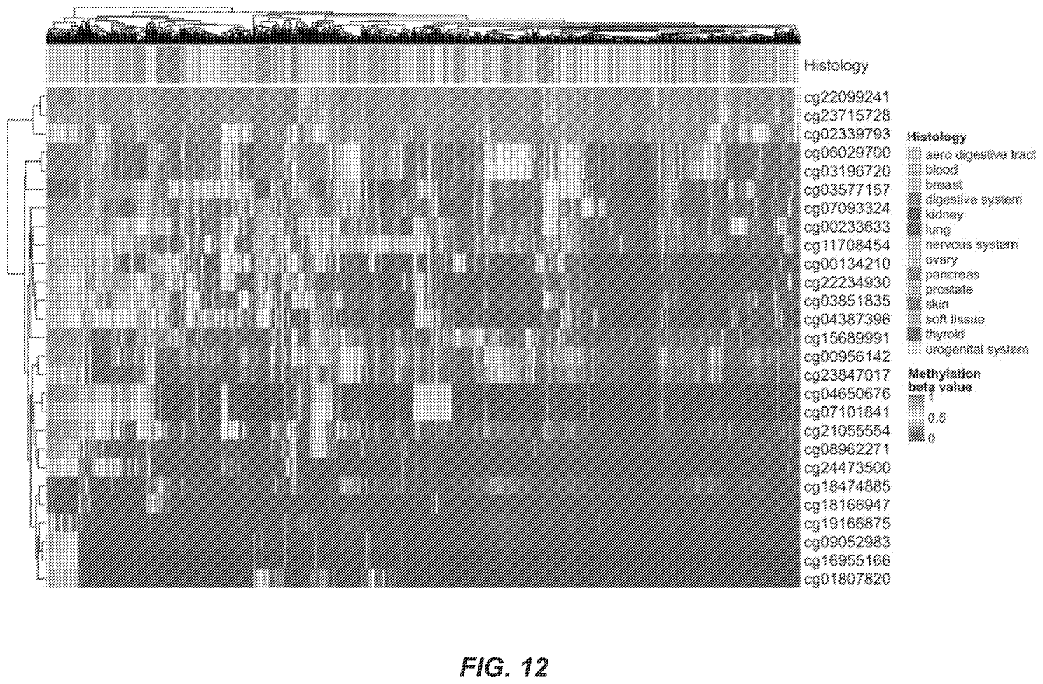

Finally, to analyze possible overlap with other tumor entities, the markers were also analyzed on the pan-cancer TCGA set (FIG. 12). Interestingly, overlap was mainly seen in samples form neurological cancers. Given the neuroendocrine phenotype of SCLC and other HGNEC this result is quite expected. However, for some markers an overlap can also be seen in skin cancer samples.

In summary, the markers worked well for the selection of SCLC and other HGNEC, especially against other lung cancer entities.

TABLE 13
Markers for distinguishing SCLC from other cancer types

| | | | | | Relation to |
|---|---|---|---|---|---|
| Methylation Site | Position (GRCh 37) | Gene | Strand | CpG Island Name | CpG Island |
| cg09052983 | chr18: 52495605 | RAB27B.TSS1500 | R | chr18: 52495604- | Island |
| | | | | 52496007 | |
| cg03196720 | chr12: 6473299 | SCNN1A.GENEBODY | F | chr12: 6472660- | Island |
| | | | | 6473322 | |
| cg03851835 | chr11: 107711742 | SLC35F2.GENEBODY | R | | |
| cg23847017 | chr1: 28764854 | PHACTR4.EXON1 | F | | |
| cg06029700 | chr12: 6473311 | SCNN1A.GENEBODY | F | chr12: 6472660- | Island |
| | | | | 6473322 | |
| cg16955166 | chr18: 52495633 | RAB27B.TSS1500 | R | chr18: 52495604- | Island |
| | | | | 52496007 | |
| cg00956142 | chr1: 28765031 | PHACTR4.GENEBODY | F | | |
| cg07101841 | chr17: 75315527 | SEPT9.UTR5 | F | | |
| cg22099241 | chr6: 31379358 | MICA.GENEBODY | R | | |
| cg00233633 | chr7: 29153560 | CPVL.GENEBODY | F | | |
| cg02339793 | chr17: 79225573 | SLC38A10.GENEBODY | R | | |
| cg07093324 | chr2: 114652143 | ACTR3.GENEBODY | R | chr2: 114647669- | S_Shelf |
| | | | | 114648312 | |
| cg18166947 | chr22: 18189443 | BCL2L13.GENEBODY | R | | |
| cg21055554 | chr12: 113652889 | IQCD.UTR5 | F | | |
| cg18474885 | chr21: 46292084 | PTTG1IP.GENEBODY | R | chr21: 46292709- | N_Shore |
| | | | | 46294744 | |
| cg19166875 | chr10: 95241944 | MYOF.EXON1 | R | | |
| cg24473500 | chr7: 871436 | UNC84A.UTR5 | F | | |
| cg22234930 | chr15: 72519739 | PKM2.UTR5 | F | chr15: 72522131- | N_Shelf |
| | | | | 72524238 | |
| cg23715728 | chr6: 31379258 | MICA.GENEBODY | R | | |
| cg04650676 | chr17: 75315486 | SEPT9.UTR5 | F | | |
| cg00134210 | chr10: 14644132 | FAM107B.GENEBODY | R | chr10: 14645752- | N_Shore |
| | | | | 14646705 | |
| cg04387396 | chr10: 72593165 | SGPL1.GENEBODY | F | | |
| cg01807820 | chr12: 6473007 | SCNN1A.GENEBODY | R | chr12: 6472660- | Island |
| | | | | 6473322 | |
| cg15689991 | chr12: 65019329 | RASSF3.GENEBODY | R | | |
| cg03577157 | chr5: 80716263 | RNUSE.GENEBODY | F | | |
| cg11708454 | chr19: 38794664 | YIF1B.UTR3 | F | | |
| cg08962271 | chr11: 67273496 | PITPNM1.TSS1500 | R | chr11: 67275067- | N_Shore |
| | | | | 67276431 | |

### Example 3—Association of Drug Response with Methylation Markers

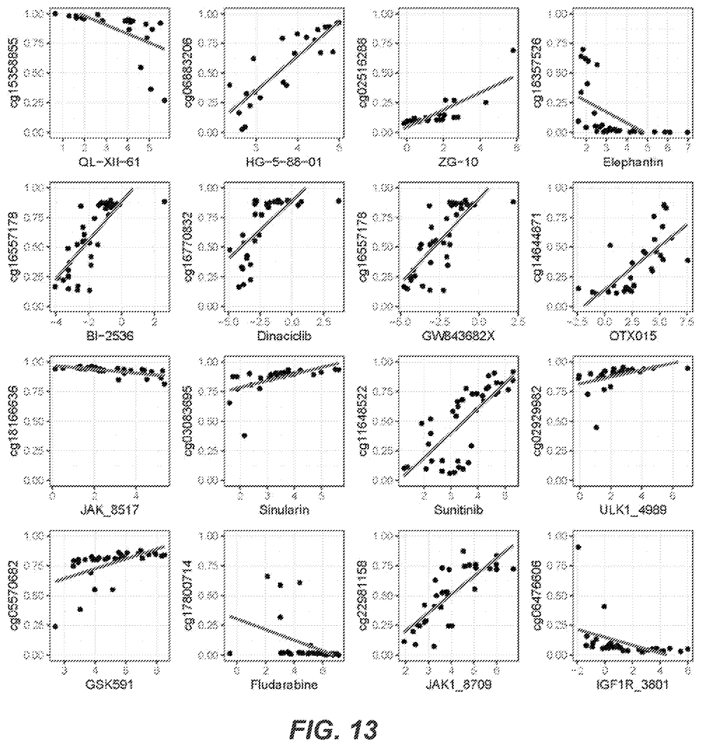

Association of methylation sites with response to treatment was assessed using the GDSC dataset that comprises ˜480,000 methylation sites with samples tested across 449 different drugs. IC₅₀ value (as a measurement of sensitivity) was correlated with methylation level using Spearman correlation. The top 16 associations between methylation sites and drugs are highlighted in FIG. 13 and shown in in Table 14.

TABLE 14

| | | | | | Spearman | |
|---|---|---|---|---|---|---|
| Methylation | | | | Spearman | Adjusted | |
| Site | Position (GRCh 37) | Gene | Drug Name | Correlation | p value | R² |
| cg15358855 | chr11:129830237 | PRDM10.GENEBODY | QL-XII-61 | −0.901750423 | 0.004869906 | 0.813154 |
| cg06883206 | chr4:152172102 | . | HG-5-88-01 | 0.868577075 | 0.037497804 | 0.754426 |
| cg02516288 | chr1:155177783 | MTX1.TSS1500 | ZG-10 | 0.867588933 | 0.016753958 | 0.752711 |
| cg18357526 | chr6:26021779 | HIST1H4A.TSS200 | Elephantin | −0.857771261 | 0.000172795 | 0.735772 |
| cg16557178 | chr17:46804309 | HOXB13.GENEBODY | BI-2536 | 0.822267206 | 6.65E−05 | 0.676123 |
| cg16770832 | chr14:56604516 | PELI2.GENEBODY | Dinaciclib | 0.814516129 | 0.006829969 | 0.663437 |
| cg16557178 | chr17:46804309 | HOXB13.GENEBODY | GW843682X | 0.812348178 | 0.000120916 | 0.65991 |
| cg14644871 | chr7:26192756 | NFE2L3.GENEBODY | OTX015 | 0.805718475 | 0.012850952 | 0.649182 |
| cg18166636 | chr5:178956931 | . | JAK_8517 | −0.805351906 | 0.013184766 | 0.648592 |
| cg03083695 | chr9:130659142 | ST6GALNAC6.GENEBODY | Sinularin | 0.799486804 | 0.019731269 | 0.639179 |
| cg11648522 | chr1:15272238 | KIAA1026.TSS200 | Sunitinib | 0.795546559 | 0.000593673 | 0.632894 |
| cg02929982 | chr14:63872285 | PPP2R5E.GENEBODY | ULK1_4989 | 0.795454545 | 0.013889773 | 0.632748 |
| cg05570682 | chr8:42132866 | IKBKB.GENEBODY | GSK591 | 0.795120321 | 0.015770449 | 0.632216 |
| cg17800714 | chrX:153656771 | ATP6AP1.TSS1500 | Fludarabine | −0.793327838 | 0.029713803 | 0.629369 |
| cg22981158 | chr19:47156401 | DACT3.GENEBODY | JAK1_8709 | 0.788489736 | 0.040598825 | 0.621716 |
| cg06476606 | chr10:91061728 | IFIT2.UTR5 | IGF1R_3801 | −0.785557185 | 0.030394645 | 0.6171 |

### Example 4—Identification of Methylation Markers Associated with SCLC Subtypes Based on Reduced Representation Bisulfite Sequencing (RRBS) Analysis of Lung Cancer Cell Lines and Patient-Derived Xenograft Samples

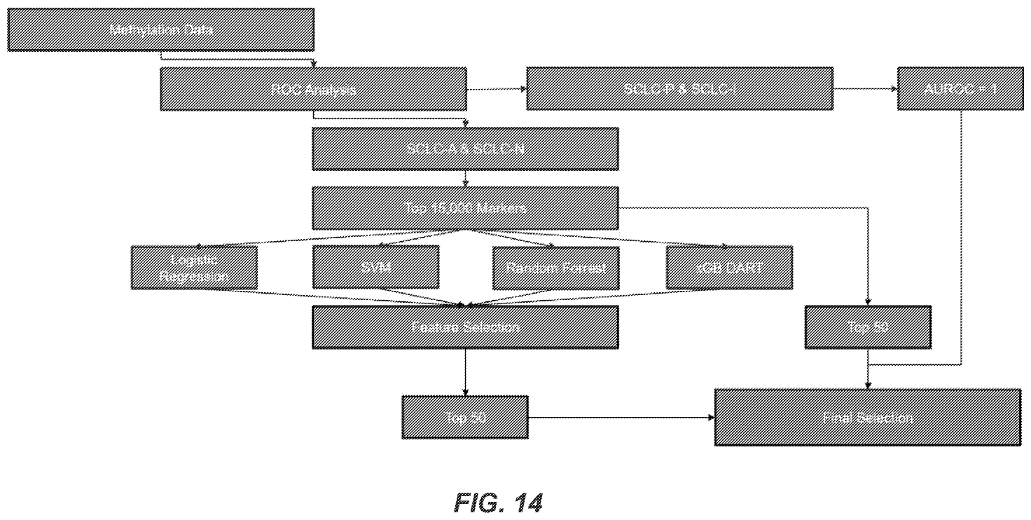

Data was generated from 59 SCLC cell lines and 68 patient derived xenograft models. Reduced representation bisulfite sequencing (RRBS) was performed at the MD Anderson Cancer Center and approximately 1-2 million methylation sites were analyzed as already described in Example 1. Briefly, receiver-operator characteristics (ROC) was used to assess the association of each of the methylation sites with one of the subtypes. A higher area under the curve (AUC) indicated a stronger association of a methylation site with the subtype. For the SCLC-A and the SCLC-N subtype, the top 15,000 methylation sites based on their AUC were further evaluated using different machine learning approaches (logistic regression, support vector machines, random forest and gradient boosting) and feature selection was used to further select methylation sites that were of high importance in the respective models. For the SCLC-P and the SCLC-I subset, limited cell line data protracted the use of machine learning models, however, many methylation sites demonstrated an AUC of 1 indicating a perfect specificity of the sites for each of the subtypes and all the sites with AUC=1 were further evaluated. A graphical overview on the analysis scheme is given in FIG. 14. The full list of methylation sites were further evaluated for their suitability in a liquid biopsy assay, as described in Example 1. For the assessment of tissue-specific methylation as well as the methylation in cfDNA, data from the Encode project were used and retrieved under the GEO accession number GSE27584. cfDNA was retrieved from array express under the accession number E-MTAB-8858. Methylation sites that were considered suitable for a liquid biopsy assay are provided in Tables 15-18.

In an additional analysis, differentially methylated sites able to differentiate the SCLC-A and the SCLC-N subsets were retrieved from the xenograft models complementing the list already obtained from the cell line data. Only methylation sites with an AUC=1 were included and further validated for their suitability in a liquid biopsy assay. The selected methylation sites are provided in Table 19.

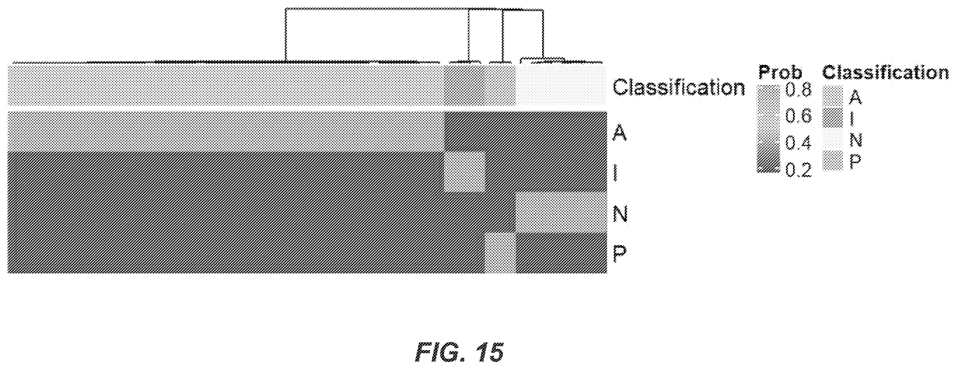

The methylation sites were validated by generating machine learning models as highlighted in the initial application. First, the methylation sites were validated on cell line data. As highlighted in FIG. 15, the selected methylation sites served to build a logistic regression models that were able to perfectly distinguish the different SCLC-Subtypes.

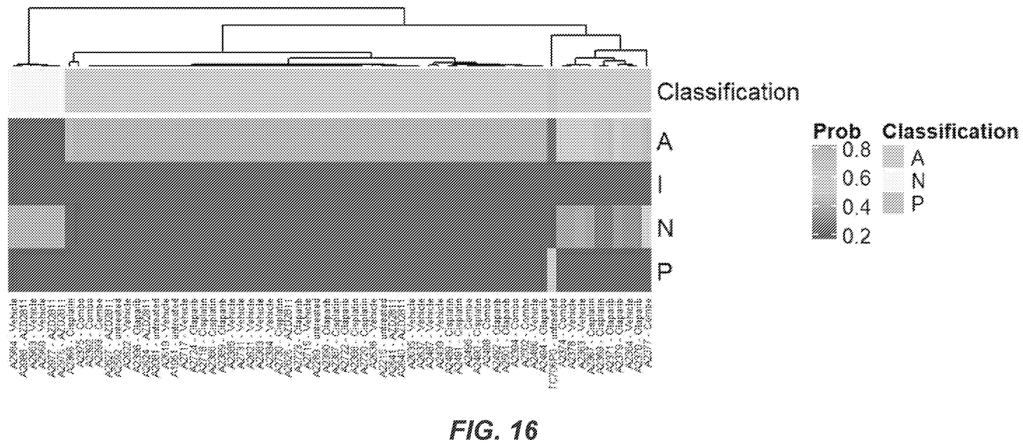

The inventors also validated the same logistic regression model on the xenograft samples as highlighted in FIG. 16. The classification was very good, with a very good separation of SCLC-A and SCLC-N. There was some overlap between the two subsets, however, SCLC-A and SCLC-N portions can be present in the same sample and the subtypes can change over time, thus it seems that the models also recapitulate this state. Importantly, while there was only one SCLC-P sample in the dataset, this was correctly classified. Consequently, the identified methylation sites do not only classify cell lines but also patient derived tumor tissue.

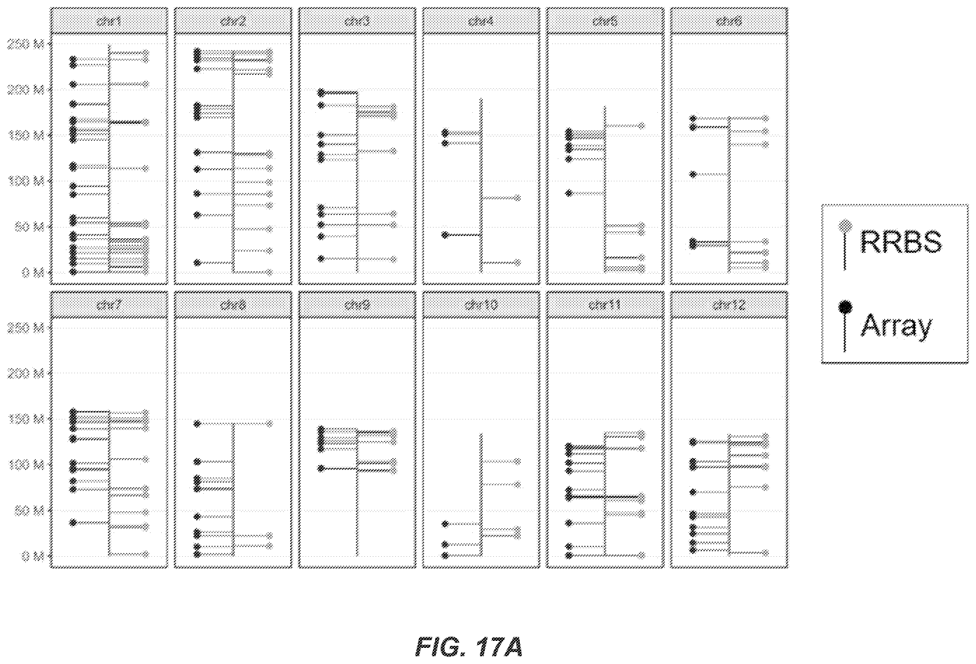

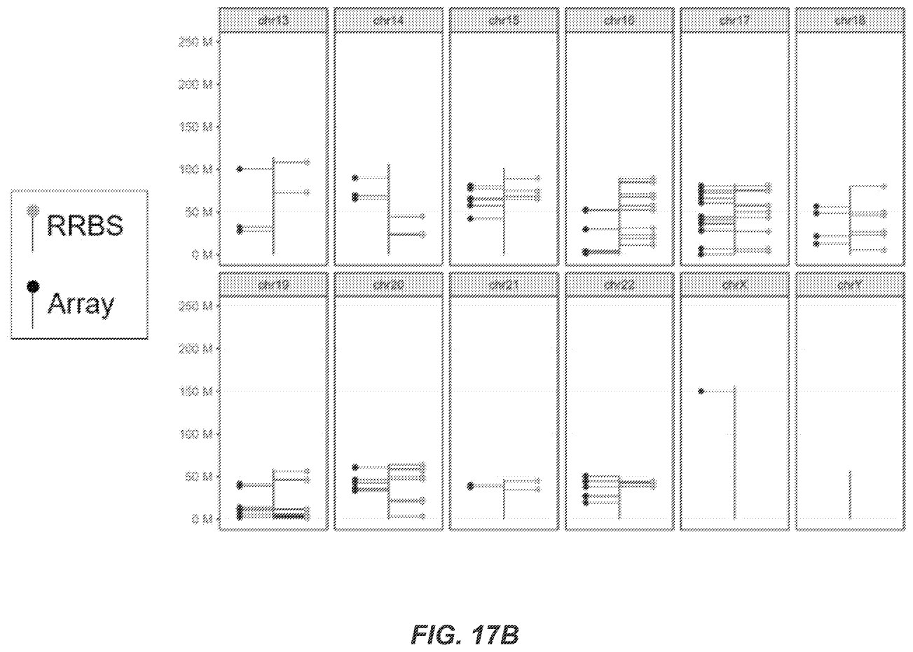

In summary, the methylation sites derived from RRBS showed comparable performance to the methylation sites derived from the microarray data and complement them by adding additional sites. The distribution of the methylation sites in the final selection of markers considered suitable for a liquid biopsy assay are highlighted in FIGS. 17A-17B (see also. All the methylation sites are evenly distributed across the chromosome.

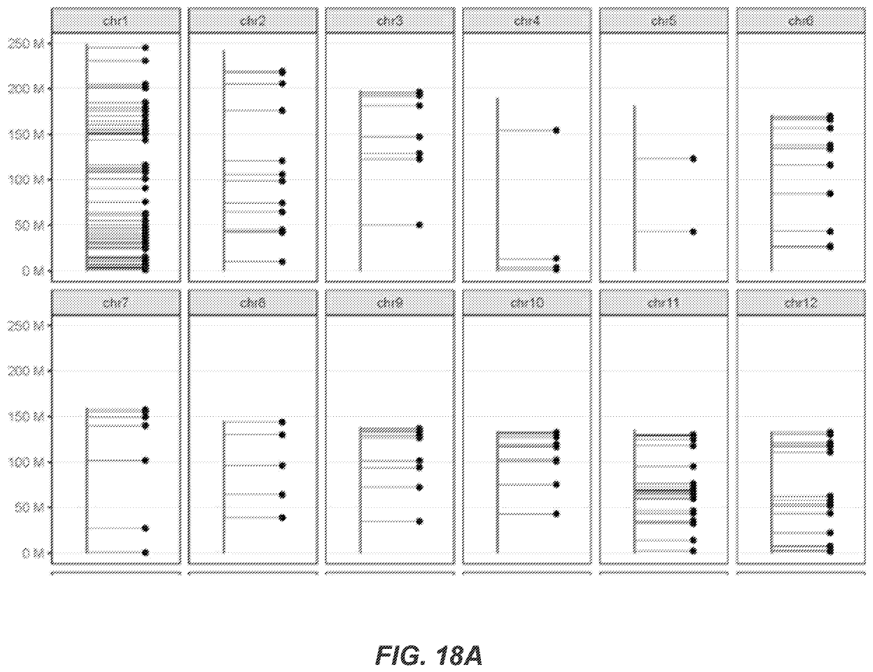

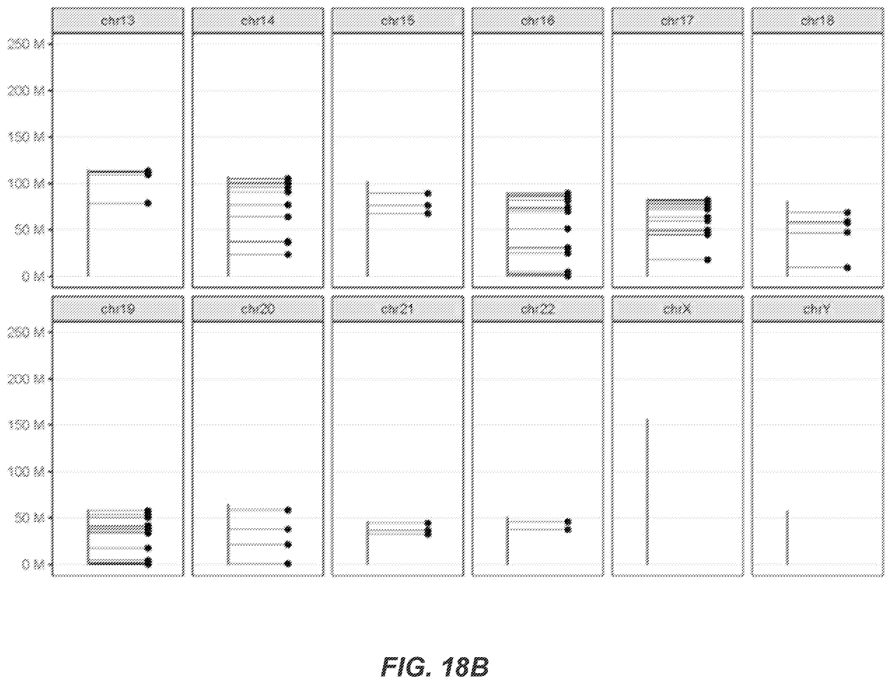

Lastly, while the methylation in cell lines strongly correlates with the methylation in patient-derived samples, did an additional analysis was done based on the xenograft models to further enhance the classification of those two subtypes and to include additional methylation sites. In total, 26283 methylation sites with an AUC=1 were detected and were further validated. A special emphasis was put on selecting regions comprising multiple methylation sites as this may be more suitable for an assay that can span multiple methylation sites at once. The distribution of the newly selected sites is highlighted in FIGS. 18A-18B (see Table 19).

TABLE 15
Methylation sites associated with SCLC-A identified from RRBS suitable for a liquid biopsy assay

| Methylation Site | GENE SYMBOL | Annotation |
|---|---|---|
| chr7: 73742662 | ABHD11 | Distal Intergenic |
| chr7: 31082303 | ADCYAP1R1 | Intron (ENST00000409363.5/117, intron 4 of 13) |
| chr16: 89359643 | ANKRD11 | Intron (ENST00000646166.1/29123, intron 3 of 4) |
| chr16: 89359653 | ANKRD11 | Intron (ENST00000646166.1/29123, intron 3 of 4) |
| chr19: 1083053 | ARHGAP45 | Promoter (<=1 kb) |
| chr17: 3959798 | ATP2A3 | Intron (ENST00000309890.11/489, intron 1 of 21) |
| chr10: 28936225 | C10orf126 | Intron (ENST00000646289.1/101929218, intron 7 of 7) |
| chr17: 81224031 | CEP131 | Promoter (1-2 kb) |
| chr17: 81223869 | CEP131 | Promoter (<=1 kb) |
| chr7: 73831674 | CLDN4 | Promoter (<=1 kb) |
| chr7: 139614241 | CLEC2L | Intron (ENST00000406875.8/28996, intron 8 of 14) |
| chr16: 23403661 | COG7 | Intron (ENST00000307149.10/91949, intron 13 of 16) |
| chr17: 57874806 | CUEDC1 | Promoter (2-3 kb) |
| chr17: 57874798 | CUEDC1 | Promoter (2-3 kb) |
| chr17: 57874857 | CUEDC1 | Promoter (2-3 kb) |
| chr17: 57874856 | CUEDC1 | Promoter (2-3 kb) |
| chr17: 57874868 | CUEDC1 | Promoter (2-3 kb) |
| chr17: 57874728 | CUEDC1 | Promoter (2-3 kb) |
| chr17: 57874706 | CUEDC1 | Promoter (2-3 kb) |
| chr17: 57874660 | CUEDC1 | Promoter (1-2 kb) |
| chr2: 241762449 | D2HGDH | Intron (ENST00000486953.5/728294, intron 3 of 3) |
| chr6: 168372036 | DACT2 | Distal Intergenic |
| chr3: 132554688 | DNAJC13 | Distal Intergenic |
| chr3: 132554514 | DNAJC13 | Distal Intergenic |
| chr2: 73324598 | EGR4 | Distal Intergenic |
| chr2: 47370087 | EPCAM | Promoter (<=1 kb) |
| chr9: 134962684 | FCN1 | Distal Intergenic |
| chr11: 64082017 | FLRT1 | Exon (ENST00000544948.1/ENST00000544948.1, exon 1 of 3) |
| chr3: 172214285 | FNDC3B | Intron (ENST00000415807.7/64778, intron 3 of 25) |
| chr17: 74961036 | HID1 | Promoter (<=1 kb) |
| chr17: 74961013 | HID1 | Promoter (<=1 kb) |
| chr2: 128299665 | HS6ST1 | Intron (ENST00000469019.1/9394, intron 1 of 2) |
| chr2: 98539557 | INPP4A | Exon (ENST00000409016.8/3631, exon 10 of 26) |
| chr20: 20364993 | INSM1 | Exon (ENST00000623641.1/ENST00000623641.1, exon 1 of 1) |
| chr20: 3223598 | ITPA | Promoter (1-2 kb) |
| chr20: 3223530 | ITPA | Promoter (1-2 kb) |
| chr20: 3223525 | ITPA | Promoter (1-2 kb) |
| chr2: 113974997 | LINC01191 | Intron (ENST00000663402.1/440900, intron 1 of 2) |
| chr21: 34743983 | LINC01426 | Promoter (1-2 kb) |
| chr20: 22678481 | LINC01747 | Intron (ENST00000634603.2/105372566, intron 2 of 2) |
| chr20: 22652808 | LINC01747 | Distal Intergenic |
| chr20: 22675604 | LINC01747 | Intron (ENST00000634603.2/105372566, intron 2 of 2) |
| chr11: 44963352 | LINC02685 | Distal Intergenic |
| chr7: 66664590 | LOC100996437 | Intron (ENST00000503687.2/27342, intron 3 of 12) |
| chr9: 103687100 | LOC101928523 | Distal Intergenic |
| chr2: 216985900 | LOC105373876 | Intron (ENST00000447289.1/ENST00000447289.1, intron 3 of 3) |
| chr2: 216986079 | LOC105373876 | Intron (ENST00000447289.1/ENST00000447289.1, intron 3 of 3) |
| chr16: 11391854 | LOC400499 | Promoter (<=1 kb) |
| chr11: 65551228 | LTBP3 | Promoter (<=1 kb) |
| chr11: 65551199 | LTBP3 | Promoter (<=1 kb) |
| chr7: 2123748 | MAD1L1 | Intron (ENST00000265854.11/8379, intron 9 of 16) |
| chr16: 84519935 | MEAK7 | Intron (ENST00000565079.5/57707, intron 1 of 3) |
| chr9: 93819287 | MIR4291 | Promoter (<=1 kb) |
| chr9: 93819136 | MIR4291 | Promoter (<=1 kb) |
| chr9: 93819135 | MIR4291 | Promoter (<=1 kb) |
| chr7: 157017814 | MNX1-AS1 | Distal Intergenic |
| chr17: 75910594 | MRPL38 | Promoter (<=1 kb) |
| chr5: 43604106 | NNT | Promoter (<=1 kb) |
| chr1: 944700 | NOC2L | Promoter (<=1 kb) |
| chr8: 22024463 | NPM2 | Promoter (<=1 kb) |
| chr1: 163692381 | NUF2 | Distal Intergenic |
| chr22: 44049781 | PARVB | Intron (ENST00000406477.7/29780, intron 2 of 13) |
| chr19: 3656261 | PIP5K1C | Intron (ENST00000335312.7/23396, intron 6 of 17) |
| chr19: 3656352 | PIP5K1C | Intron (ENST00000335312.7/23396, intron 6 of 17) |
| chr19: 3656274 | PIP5K1C | Intron (ENST00000335312.7/23396, intron 6 of 17) |
| chr3: 64225316 | PRICKLE2 | Promoter (<=1 kb) |
| chr9: 131721355 | RAPGEF1 | Intron (ENST00000372195.5/2889, intron 1 of 23) |
| chr9: 131721356 | RAPGEF1 | Intron (ENST00000372195.5/2889, intron 1 of 23) |
| chr12: 97321085 | RMST | Distal Intergenic |
| chr10: 78139388 | RPS24 | Distal Intergenic |
| chr20: 46641000 | SLC13A3 | Intron (ENST00000372121.5/64849, intron 1 of 6) |
| chr1: 205965076 | SLC26A9 | Distal Intergenic |
| chr1: 205965024 | SLC26A9 | Distal Intergenic |
| chr6: 168440498 | SMOC2 | Promoter (<=1 kb) |
| chr17: 43755336 | SOST | 3′ UTR |
| chr3: 181315137 | SOX2-OT | Intron (ENST00000643236.2/347689, intron 3 of 5) |
| chr1: 54356529 | SSBP3 | Promoter (<=1 kb) |
| chr1: 36366642 | STK40 | Promoter (<=1 kb) |
| chr1: 36366474 | STK40 | Promoter (<=1 kb) |
| chr12: 124088409 | ZNF664 | Exon (ENST00000621669.1/ENST00000621669.1, exon 1 of 1) |
| chr11: 60866977 | ZP1 | Promoter (<=1 kb) |

TABLE 16
Methylation sites associated with SCLC-N identified
from RRBS suitable for a liquid biopsy assay

| Methylation Site | GENE SYMBOL | Annotation |
|---|---|---|
| chr16: 57620421 | ADGRG1 | Promoter (<=1 kb) |
| chr7: 105689129 | ATXN7L1 | Promoter (2-3 kb) |
| chr9: 134051444 | BRD3 | Promoter (1-2 kb) |
| chr17: 50604550 | CACNA1G | Intron (ENST00000359106.10/8913, intron 22 of 37) |
| chr14: 24311062 | CIDEB | Promoter (<=1 kb) |
| chr15: 68211219 | CLN6 | Promoter (1-2 kb) |
| chr17: 57874862 | CUEDC1 | Promoter (2-3 kb) |
| chr1: 28869236 | EPB41 | 3′ UTR |
| chr5: 160245925 | FABP6 | Distal Intergenic |
| chr6: 154359428 | IPCEF1 | Promoter (2-3 kb) |
| chr15: 68347588 | ITGA11 | Intron (ENST00000315757.9/22801, intron 10 of 29) |
| chr15: 68347636 | ITGA11 | Intron (ENST00000315757.9/22801, intron 10 of 29) |
| chr7: 150975167 | KCNH2 | Promoter (2-3 kb) |
| chr19: 5035647 | KDM4B | Intron (ENST00000611640.4/23030, intron 3 of 23) |
| chr16: 67178561 | KIAA0895L | Promoter (<=1 kb) |
| chr16: 67178592 | KIAA0895L | Promoter (<=1 kb) |
| chr16: 85287162 | LINC00311 | Intron (ENST00000637419.1/23199, intron 1 of 2) |
| chr7: 66664578 | LOC100996437 | Intron (ENST00000503687.2/27342, intron 3 of 12) |
| chr7: 66664596 | LOC100996437 | Intron (ENST00000503687.2/27342, intron 3 of 12) |
| chr7: 66664710 | LOC100996437 | Intron (ENST00000503687.2/27342, intron 3 of 12) |
| chr1: 53320090 | LRP8 | Intron (ENST00000306052.12/7804, intron 2 of 18) |
| chr1: 25771820 | MAN1C1 | Promoter (<=1 kb) |
| chr8: 144508436 | MFSD3 | Promoter (<=1 kb) |
| chr6: 33694527 | MIR3934 | Exon (ENST00000606961.1/ENST00000606961.1, exon 1 of 1) |
| chr12: 124428932 | NCOR2 | Promoter (1-2 kb) |
| chr3: 52468694 | NISCH | Promoter (1-2 kb) |
| chr9: 124820937 | OLFML2A | Distal Intergenic |
| chr22: 44049853 | PARVB | Intron (ENST00000406477.7/29780, intron 2 of 13) |
| chr19: 3656250 | PIP5K1C | Intron (ENST00000335312.7/23396, intron 6 of 17) |
| chr19: 3656275 | PIP5K1C | Intron (ENST00000335312.7/23396, intron 6 of 17) |
| chr19: 3656172 | PIP5K1C | Intron (ENST00000335312.7/23396, intron 6 of 17) |
| chr19: 3656178 | PIP5K1C | Intron (ENST00000335312.7/23396, intron 6 of 17) |
| chr19: 3656369 | PIP5K1C | Intron (ENST00000335312.7/23396, intron 6 of 17) |
| chr19: 3655804 | PIP5K1C | Intron (ENST00000335312.7/23396, intron 6 of 17) |
| chr19: 3655921 | PIP5K1C | Intron (ENST00000335312.7/23396, intron 6 of 17) |
| chr2: 264135 | SH3YL1 | Promoter (<=1 kb) |
| chr1: 232526599 | SIPA1L2 | Intron (ENST00000366630.5/57568, intron 1 of 21) |
| chr1: 54356528 | SSBP3 | Promoter (<=1 kb) |
| chr1: 36366641 | STK40 | Promoter (<=1 kb) |
| chr6: 10779357 | TMEM14B | Intron (ENST00000480294.1/221711, intron 3 of 18) |
| chr20: 50015246 | TRERNA1 | Distal Intergenic |
| chr16: 70730157 | VAC14 | Intron (ENST00000568886.5/55697, intron 8 of 12) |

TABLE 17
Methylation sites associated with SCLC-P identified from RRBS suitable for a liquid biopsy assay

| Methylation Site | GENE SYMBOL | Annotation |
|---|---|---|
| chr1: 33315055 | A3GALT2 | Intron (ENST00000588828.1/ENST00000588828.1, intron 1 of 1) |
| chr1: 33315062 | A3GALT2 | Intron (ENST00000588828.1/ENST00000588828.1, intron 1 of 1) |
| chr1: 33315063 | A3GALT2 | Intron (ENST00000588828.1/ENST00000588828.1, intron 1 of 1) |
| chr1: 33315101 | A3GALT2 | Intron (ENST00000588828.1/ENST00000588828.1, intron 1 of 1) |
| chr22: 42757869 | A4GALT | Distal Intergenic |
| chr22: 42757892 | A4GALT | Distal Intergenic |
| chr14: 22984092 | AJUBA | Promoter (1-2 kb) |
| chr14: 22984101 | AJUBA | Promoter (1-2 kb) |
| chr14: 22984111 | AJUBA | Promoter (1-2 kb) |
| chr14: 22984144 | AJUBA | Promoter (1-2 kb) |
| chr18: 26652681 | AQP4-AS1 | Intron (ENST00000580191.5/284252, intron 1 of 4) |
| chr12: 110245057 | ATP2A2 | Distal Intergenic |
| chr12: 110245058 | ATP2A2 | Distal Intergenic |
| chr16: 31109235 | BCKDK | Promoter (<=1 kb) |
| chr1: 15207848 | C1orf195 | Intron (ENST00000428417.5/55092, intron 1 of 2) |
| chr10: 103479364 | CALHM3 | Promoter (<=1 kb) |
| chr12: 75306272 | CAPS2 | Exon (ENST00000547243.1/ENST00000547243.1, exon 1 of 1) |
| chr6: 21894804 | CASC15 | Intron (ENST00000671160.1/401237, intron 2 of 7) |
| chr6: 21894816 | CASC15 | Intron (ENST00000671160.1/401237, intron 2 of 7) |
| chr1: 50977120 | CDKN2C | Distal Intergenic |
| chr11: 117794285 | DSCAML1 | Promoter (2-3 kb) |
| chr11: 117794300 | DSCAML1 | Promoter (2-3 kb) |
| chr11: 117794312 | DSCAML1 | Promoter (2-3 kb) |
| chr10: 22229200 | EBLN1 | Distal Intergenic |
| chr10: 22229203 | EBLN1 | Distal Intergenic |
| chr10: 22229207 | EBLN1 | Distal Intergenic |
| chr10: 22229209 | EBLN1 | Distal Intergenic |
| chr10: 22229211 | EBLN1 | Distal Intergenic |
| chr10: 22229213 | EBLN1 | Distal Intergenic |
| chr19: 11483715 | ELAVL3 | Promoter (2-3 kb) |
| chr19: 11483744 | ELAVL3 | Promoter (2-3 kb) |
| chr19: 11483821 | ELAVL3 | Promoter (2-3 kb) |
| chr19: 11483824 | ELAVL3 | Promoter (2-3 kb) |
| chr19: 11483826 | ELAVL3 | Promoter (2-3 kb) |
| chr19: 11483828 | ELAVL3 | Promoter (2-3 kb) |
| chr19: 11483877 | ELAVL3 | Promoter (2-3 kb) |
| chr19: 11483882 | ELAVL3 | Promoter (2-3 kb) |
| chr19: 11483887 | ELAVL3 | Promoter (2-3 kb) |
| chr19: 11483902 | ELAVL3 | Promoter (2-3 kb) |
| chr19: 11483903 | ELAVL3 | Promoter (2-3 kb) |
| chr19: 11483933 | ELAVL3 | Promoter (2-3 kb) |
| chr18: 5543758 | EPB41L3 | Promoter (<=1 kb) |
| chr1: 11636037 | FBXO2 | Intron (ENST00000642131.1/ENST00000642131.1, intron 1 of 1) |
| chr1: 239997697 | FMN2 | Distal Intergenic |
| chr1: 240092877 | FMN2 | Promoter (<=1 kb) |
| chr1: 240092884 | FMN2 | Promoter (1-2 kb) |
| chr9: 101738424 | GRIN3A | Promoter (<=1 kb) |
| chr9: 101738450 | GRIN3A | Promoter (<=1 kb) |
| chr12: 120967113 | HNF1A-AS1 | Intron (ENST00000619441.1/ENST00000619441.1, intron 1 of 1) |
| chr5: 5480537 | ICE1 | Intron (ENST00000296564.9/23379, intron 17 of 18) |
| chr5: 5480539 | ICE1 | Intron (ENST00000296564.9/23379, intron 17 of 18) |
| chr5: 2751058 | IRX2 | Promoter (<=1 kb) |
| chr5: 2751201 | IRX2 | Promoter (<=1 kb) |
| chr16: 19130411 | ITPRIPL2 | Intron (ENST00000568526.1/162073, intron 2 of 4) |
| chr11: 134072658 | JAM3 | Intron (ENST00000534549.5/83700, intron 1 of 2) |
| chr11: 134072677 | JAM3 | Intron (ENST00000534549.5/83700, intron 1 of 2) |
| chr7: 32907550 | KBTBD2 | Distal Intergenic |
| chr17: 75034158 | KCTD2 | Promoter (1-2 kb) |
| chr2: 23667161 | KLHL29 | Intron (ENST00000486442.6/114818, intron 5 of 13) |
| chr6: 21423591 | LINC00581 | Distal Intergenic |
| chr18: 49655943 | LIPG | Distal Intergenic |
| chr20: 57407195 | LOC100291105 | Intron (ENST00000440234.6/55544, intron 2 of 2) |
| chr5: 51383057 | LOC642366 | Promoter (<=1 kb) |
| chr5: 51383086 | LOC642366 | Promoter (<=1 kb) |
| chr19: 5704447 | LONP1 | Intron (ENST00000587552.5/9361, intron 7 of 15) |
| chr6: 5213923 | LYRM4 | Promoter (2-3 kb) |
| chr1: 25603895 | MAN1C1 | Distal Intergenic |
| chr1: 25603905 | MAN1C1 | Distal Intergenic |
| chr5: 16179849 | MARCHF11 | Promoter (<=1 kb) |
| chr5: 16179857 | MARCHF11 | Promoter (<=1 kb) |
| chr5: 16179865 | MARCHF11 | Promoter (<=1 kb) |
| chr5: 16179878 | MARCHF11 | Promoter (<=1 kb) |
| chr5: 16179881 | MARCHF11 | Promoter (<=1 kb) |
| chr5: 16179883 | MARCHF11 | Promoter (<=1 kb) |
| chr5: 16179960 | MARCHF11 | Promoter (<=1 kb) |
| chr2: 239344172 | MIR2467 | Intron (ENST00000345617.7/9759, intron 2 of 26) |
| chr2: 239344189 | MIR2467 | Intron (ENST00000345617.7/9759, intron 2 of 26) |
| chr15: 74789232 | MIR4513 | Promoter (<=1 kb) |
| chr17: 27293362 | MIR4522 | Promoter (<=1 kb) |
| chr15: 89389866 | MIR9-3HG | Promoter (2-3 kb) |
| chr13: 108495844 | MYO16 | Distal Intergenic |
| chr13: 108495845 | MYO16 | Distal Intergenic |
| chr13: 108495846 | MYO16 | Distal Intergenic |
| chr13: 108495847 | MYO16 | Distal Intergenic |
| chr13: 108495848 | MYO16 | Distal Intergenic |
| chr1: 164576888 | PBX1 | Promoter (<=1 kb) |
| chr1: 164576889 | PBX1 | Promoter (<=1 kb) |
| chr1: 164576897 | PBX1 | Promoter (<=1 kb) |
| chr1: 164576907 | PBX1 | Promoter (<=1 kb) |
| chr1: 164576909 | PBX1 | Promoter (<=1 kb) |
| chr3: 170182685 | PHC3 | Promoter (<=1 kb) |
| chr1: 113758109 | PHTF1 | Promoter (1-2 kb) |
| chr1: 113758121 | PHTF1 | Promoter (1-2 kb) |
| chr13: 72840708 | PIBF1 | Intron (ENST00000615625.1/10464, intron 2 of 8) |
| chr1: 6470978 | PLEKHG5 | Promoter (<=1 kb) |
| chr1: 6470979 | PLEKHG5 | Promoter (<=1 kb) |
| chr1: 6470980 | PLEKHG5 | Promoter (<=1 kb) |
| chr1: 6470981 | PLEKHG5 | Promoter (<=1 kb) |
| chr1: 6470982 | PLEKHG5 | Promoter (<=1 kb) |
| chr1: 6471004 | PLEKHG5 | Promoter (<=1 kb) |
| chr1: 6471154 | PLEKHG5 | Promoter (<=1 kb) |
| chr1: 6471412 | PLEKHG5 | Promoter (<=1 kb) |
| chr3: 52167456 | POC1A | Distal Intergenic |
| chr12: 3491095 | PRMT8 | Promoter (<=1 kb) |
| chr12: 3491108 | PRMT8 | Promoter (<=1 kb) |
| chr12: 3491115 | PRMT8 | Promoter (<=1 kb) |
| chr18: 46028443 | PSTPIP2 | Exon (ENST00000591191.1/ENST00000591191.1, exon 1 of 1) |
| chr2: 130372287 | PTPN18 | Promoter (<=1 kb) |
| chr2: 130372663 | PTPN18 | Promoter (<=1 kb) |
| chr2: 130372713 | PTPN18 | Promoter (<=1 kb) |
| chr2: 130372851 | PTPN18 | Promoter (<=1 kb) |
| chr1: 21664061 | RAP1GAP | Intron (ENST00000290101.8/5909, intron 1 of 26) |
| chr1: 21664104 | RAP1GAP | Intron (ENST00000290101.8/5909, intron 1 of 26) |
| chr1: 21664105 | RAP1GAP | Intron (ENST00000290101.8/5909, intron 1 of 26) |
| chr20: 63689809 | RTEL1 | Promoter (<=1 kb) |
| chr20: 63689822 | RTEL1 | Promoter (<=1 kb) |
| chr9: 136451683 | SEC16A | Promoter (2-3 kb) |
| chr9: 136451687 | SEC16A | Promoter (2-3 kb) |
| chr2: 85429488 | SH2D6 | Promoter (<=1 kb) |
| chr11: 47406945 | SLC39A13 | Promoter (<=1 kb) |
| chr15: 65045932 | SLC51B | Promoter (<=1 kb) |
| chr3: 14401986 | SLC6A6 | Promoter (<=1 kb) |
| chr12: 130788705 | STX2 | Downstream (<1 kb) |
| chr7: 48002285 | SUN3 | Promoter (2-3 kb) |
| chr12: 109924012 | TCHP | Intron (ENST00000544838.5/84260, intron 13 of 14) |
| chr12: 109924015 | TCHP | Intron (ENST00000544838.5/84260, intron 13 of 14) |
| chr12: 109924087 | TCHP | Intron (ENST00000544838.5/84260, intron 13 of 14) |
| chr12: 109924155 | TCHP | Intron (ENST00000544838.5/84260, intron 13 of 14) |
| chr12: 109924248 | TCHP | Intron (ENST00000544838.5/84260, intron 13 of 14) |
| chr1: 35578285 | TFAP2E | Promoter (<=1 kb) |
| chr18: 23282431 | TMEM241 | Intron (ENST00000578790.5/85019, intron 2 of 3) |
| chr1: 6460633 | TNFRSF25 | Promoter (1-2 kb) |
| chr1: 6460653 | TNFRSF25 | Promoter (1-2 kb) |
| chr1: 6460894 | TNFRSF25 | Promoter (1-2 kb) |
| chr1: 6460918 | TNFRSF25 | Promoter (1-2 kb) |
| chr1: 6460934 | TNFRSF25 | Promoter (1-2 kb) |
| chr1: 6460935 | TNFRSF25 | Promoter (1-2 kb) |
| chr1: 6460947 | TNFRSF25 | Promoter (1-2 kb) |
| chr1: 6460956 | TNFRSF25 | Promoter (1-2 kb) |
| chr1: 6461061 | TNFRSF25 | Promoter (1-2 kb) |
| chr1: 6461087 | TNFRSF25 | Promoter (1-2 kb) |
| chr1: 6461096 | TNFRSF25 | Promoter (1-2 kb) |
| chr1: 6461190 | TNFRSF25 | Promoter (1-2 kb) |
| chr1: 6461192 | TNFRSF25 | Promoter (1-2 kb) |
| chr1: 6461254 | TNFRSF25 | Promoter (1-2 kb) |
| chr1: 6461265 | TNFRSF25 | Promoter (1-2 kb) |
| chr1: 6461279 | TNFRSF25 | Promoter (1-2 kb) |
| chr1: 6461288 | TNFRSF25 | Promoter (1-2 kb) |
| chr1: 6461304 | TNFRSF25 | Promoter (1-2 kb) |
| chr14: 45021208 | TOGARAM1 | Intron (ENST00000556239.5/54813, intron 1 of 2) |
| chr22: 38403885 | TPTEP2 | Distal Intergenic |
| chr22: 38403893 | TPTEP2 | Distal Intergenic |
| chr22: 38403895 | TPTEP2 | Distal Intergenic |
| chr22: 38403915 | TPTEP2 | Distal Intergenic |
| chr18: 79994148 | TXNLAA | Intron (ENST00000585474.5/10907, intron 1 of 2) |
| chr12: 122875685 | VPS37B | Exon (ENST00000623210.1/ENST00000623210.1, exon 1 of 1) |
| chr19: 56155326 | ZNF444 | Promoter (1-2 kb) |
| chr19: 11483959 | ZNF653 | Promoter (2-3 kb) |
| chr19: 11483967 | ZNF653 | Promoter (2-3 kb) |
| chr19: 11483973 | ZNF653 | Promoter (2-3 kb) |
| chr19: 56309806 | ZSCAN5A | Intron (ENST00000593106.5/79149, intron 2 of 4) |

TABLE 18
Methylation sites associated with SCLC-I identified from RRBS suitable for a liquid biopsy assay

| Methylation Site | GENE SYMBOL | Annotation |
|---|---|---|
| chr16: 52621872 | CASC16 | Intron (ENST00000652959.1/643714, intron 3 of 6) |
| chr16: 52621873 | CASC16 | Intron (ENST00000652959.1/643714, intron 3 of 6) |
| chr11: 64299647 | CATSPERZ | Promoter (<=1 kb) |
| chr11: 64299653 | CATSPERZ | Promoter (<=1 kb) |
| chr11: 64299676 | CATSPERZ | Promoter (<=1 kb) |
| chr11: 64299686 | CATSPERZ | Promoter (<=1 kb) |
| chr11: 64299688 | CATSPERZ | Promoter (<=1 kb) |
| chr4: 10717468 | CLNK | Distal Intergenic |
| chr4: 10717488 | CLNK | Distal Intergenic |
| chr7: 146562076 | CNTNAP2 | Intron (ENST00000625365.2/26047, intron 2 of 3) |
| chr7: 146735323 | CNTNAP2 | Intron (ENST00000625365.2/26047, intron 2 of 3) |
| chr7: 147640913 | CNTNAP2 | Promoter (1-2 kb) |
| chr17: 7204895 | DLG4 | Promoter (<=1 kb) |
| chr2: 232486759 | ECEL1 | Promoter (<=1 kb) |
| chr2: 232486768 | ECEL1 | Promoter (<=1 kb) |
| chr2: 221307744 | EPHA4 | Exon (ENST00000650956.1/ENST00000650956.1, exon 1 of 2) |
| chr11: 725837 | EPS8L2 | Promoter (<=1 kb) |
| chr11: 726066 | EPS8L2 | Promoter (<=1 kb) |
| chr11: 726081 | EPS8L2 | Promoter (<=1 kb) |
| chr11: 726088 | EPS8L2 | Promoter (<=1 kb) |
| chr11: 726100 | EPS8L2 | Promoter (<=1 kb) |
| chr11: 726121 | EPS8L2 | Promoter (<=1 kb) |
| chr11: 726122 | EPS8L2 | Promoter (<=1 kb) |
| chr11: 726130 | EPS8L2 | Promoter (<=1 kb) |
| chr11: 726169 | EPS8L2 | Promoter (<=1 kb) |
| chr11: 726187 | EPS8L2 | Promoter (<=1 kb) |
| chr11: 726192 | EPS8L2 | Promoter (<=1 kb) |
| chr11: 726275 | EPS8L2 | Promoter (<=1 kb) |
| chr11: 726278 | EPS8L2 | Promoter (<=1 kb) |
| chr11: 726289 | EPS8L2 | Promoter (<=1 kb) |
| chr11: 726291 | EPS8L2 | Promoter (<=1 kb) |
| chr11: 726293 | EPS8L2 | Promoter (<=1 kb) |
| chr11: 726333 | EPS8L2 | Promoter (<=1 kb) |
| chr11: 726336 | EPS8L2 | Promoter (<=1 kb) |
| chr11: 726345 | EPS8L2 | Promoter (<=1 kb) |
| chr11: 726367 | EPS8L2 | Promoter (<=1 kb) |
| chr11: 726387 | EPS8L2 | Promoter (<=1 kb) |
| chr11: 726397 | EPS8L2 | Promoter (<=1 kb) |
| chr11: 726408 | EPS8L2 | Promoter (<=1 kb) |
| chr11: 726419 | EPS8L2 | Promoter (<=1 kb) |
| chr11: 726491 | EPS8L2 | Promoter (<=1 kb) |
| chr11: 726497 | EPS8L2 | Promoter (<=1 kb) |
| chr11: 726561 | EPS8L2 | Promoter (<=1 kb) |
| chr11: 726611 | EPS8L2 | Promoter (<=1kb) |
| chr6: 139771860 | FILNC1 | Intron (ENST00000644936.1/100132735, intron 1 of 2) |
| chr21: 44931508 | ITGB2 | Promoter (<=1 kb) |
| chr8: 11059296 | LOC101929269 | Intron (ENST00000416569.3/286046, intron 1 of 2) |
| chr19: 46016130 | MIR769 | Promoter (2-3 kb) |
| chr19: 46016136 | MIR769 | Promoter (2-3 kb) |
| chr19: 46016138 | MIR769 | Promoter (2-3 kb) |
| chr19: 46016139 | MIR769 | Promoter (2-3 kb) |
| chr19: 46016142 | MIR769 | Promoter (2-3 kb) |
| chr19: 46016144 | MIR769 | Promoter (2-3 kb) |
| chr19: 46016145 | MIR769 | Promoter (2-3 kb) |
| chr19: 46016146 | MIR769 | Promoter (2-3 kb) |
| chr19: 46016147 | MIR769 | Promoter (2-3 kb) |
| chr19: 46016148 | MIR769 | Promoter (2-3 kb) |
| chr19: 46016149 | MIR769 | Promoter (2-3 kb) |
| chr3: 175259809 | NAALADL2 | Intron (ENST00000424690.1/100874244, intron 1 of 2) |
| chr3: 175854326 | NAALADL2-AS1 | Distal Intergenic |
| chr9: 93133105 | NINJ1 | Promoter (1-2 kb) |
| chr1: 163423133 | NUF2 | Exon (ENST00000430516.1/ENST00000430516.1, exon 1 of 2) |
| chr1: 163423134 | NUF2 | Exon (ENST00000430516.1/ENST00000430516.1, exon 1 of 2) |
| chr1: 163423194 | NUF2 | Distal Intergenic |
| chr1: 163423199 | NUF2 | Distal Intergenic |
| chr1: 163677704 | NUF2 | Distal Intergenic |
| chr1: 163692555 | NUF2 | Distal Intergenic |
| chr19: 2290919 | PEAK3 | Exon (ENST00000585527.1/ENST00000585527.1, exon 1 of 1) |
| chr19: 2290925 | PEAK3 | Exon (ENST00000585527.1/ENST00000585527.1, exon 1 of 1) |
| chr19: 2290978 | PEAK3 | Exon (ENST00000585527.1/ENST00000585527.1, exon 1 of 1) |
| chr19: 2290996 | PEAK3 | Exon (ENST00000585527.1/ENST00000585527.1, exon 1 of 1) |
| chr19: 2290999 | PEAK3 | Exon (ENST00000585527.1/ENST00000585527.1, exon 1 of 1) |
| chr19: 2291021 | PEAK3 | Exon (ENST00000585527.1/ENST00000585527.1, exon 1 of 1) |
| chr20: 59605599 | PHACTR3 | Promoter (1-2 kb) |
| chr20: 59605612 | PHACTR3 | Promoter (1-2 kb) |
| chr20: 59605622 | PHACTR3 | Promoter (1-2 kb) |
| chr20: 59605632 | PHACTR3 | Promoter (1-2 kb) |
| chr20: 59605688 | PHACTR3 | Promoter (1-2 kb) |
| chr20: 59605692 | PHACTR3 | Promoter (1-2 kb) |
| chr20: 59605706 | PHACTR3 | Promoter (1-2 kb) |
| chr20: 59605758 | PHACTR3 | Promoter (1-2 kb) |
| chr2: 231707590 | PTMA | Promoter (<=1 kb) |
| chr4: 81586509 | RASGEF1B | Intron (ENST00000638048.1/153020, intron 4 of 16) |
| chr12: 97403582 | RMST | Distal Intergenic |
| chr12: 97403620 | RMST | Distal Intergenic |
| chr12: 97403630 | RMST | Distal Intergenic |
| chr12: 98311179 | SLC9A7P1 | Distal Intergenic |
| chr11: 130160086 | ST14 | Promoter (<=1 kb) |
| chr16: 52395111 | TOX3 | Distal Intergenic |
| chr16: 52504937 | TOX3 | Intron (ENST00000563091.1/27324, intron 1 of 3) |
| chr16: 52505114 | TOX3 | Intron (ENST00000563091.1/27324, intron 1 of 3) |

TABLE 19
Methylation sites capable of differentiating SCLC-A
from SCLC-N identified from xenograft samples

| Methylation Site | GENE SYMBOL | Annotation |
|---|---|---|
| chr1: 1048828 | AGRN | Promoter (<=1kb) |
| chr1: 1048892 | AGRN | Promoter (<=1kb) |
| chr1: 1048899 | AGRN | Promoter (<=1kb) |
| chr1: 1048926 | AGRN | Promoter (<=1kb) |
| chr1: 1048928 | AGRN | Promoter (<=1kb) |
| chr1: 1048940 | AGRN | Promoter (<=1kb) |
| chr1: 1224106 | SDF4 | Promoter (2-3kb) |
| chr1: 1345500 | DVL1 | Intron (ENST00000378891.9/1855, intron 1 of 14) |
| chr1: 1345502 | DVL1 | Intron (ENST00000378891.9/1855, intron 1 of 14) |
| chr1: 1345520 | DVL1 | Intron (ENST00000378891.9/1855, intron 1 of 14) |
| chr1: 1345521 | DVL1 | Intron (ENST00000378891.9/1855, intron 1 of 14) |
| chr1: 2204525 | FAAP20 | Promoter (<=1kb) |
| chr1: 2204544 | FAAP20 | Promoter (<=1kb) |
| chr1: 2204588 | FAAP20 | Promoter (<=1kb) |
| chr1: 2204592 | FAAP20 | Promoter (<=1kb) |
| chr1: 2204595 | FAAP20 | Promoter (<=1kb) |
| chr1: 2204598 | FAAP20 | Promoter (<=1kb) |
| chr1: 2204676 | FAAP20 | Promoter (<=1kb) |
| chr1: 2204721 | FAAP20 | Promoter (<=1kb) |
| chr1: 2204778 | FAAP20 | Promoter (<=1kb) |
| chr1: 2204779 | FAAP20 | Promoter (<=1kb) |
| chr1: 2204782 | FAAP20 | Promoter (<=1kb) |
| chr1: 2204799 | FAAP20 | Promoter (<=1kb) |
| chr1: 2204802 | FAAP20 | Promoter (<=1kb) |
| chr1: 2306607 | SKI | Promoter (2-3kb) |
| chr1: 2306609 | SKI | Promoter (2-3kb) |
| chr1: 2306615 | SKI | Promoter (2-3kb) |
| chr1: 3101046 | MIR4251 | Intron (ENST00000511072.5/63976, intron 1 of 15) |
| chr1: 3101087 | MIR4251 | Intron (ENST00000511072.5/63976, intron 1 of 15) |
| chr1: 3238646 | PRDM16 | Promoter (<=1kb) |
| chr1: 3524220 | MEGF6 | Exon (ENST00000356575.9/1953, exon 5 of 37) |
| chr1: 3524231 | MEGF6 | Exon (ENST00000356575.9/1953, exon 5 of 37) |
| chr1: 3524238 | MEGF6 | Exon (ENST00000356575.9/1953, exon 5 of 37) |
| chr1: 3772826 | SMIM1 | Promoter (<=1kb) |
| chr1: 3772830 | SMIM1 | Promoter (<=1kb) |
| chr1: 3772845 | SMIM1 | Promoter (<=1kb) |
| chr1: 3772853 | SMIM1 | Promoter (<=1kb) |
| chr1: 3772855 | SMIM1 | Promoter (<=1kb) |
| chr1: 3772861 | SMIM1 | Promoter (<=1kb) |
| chr1: 4711117 | AJAP1 | Promoter (<=1kb) |
| chr1: 4711119 | AJAP1 | Promoter (<=1kb) |
| chr1: 6473328 | PLEKHG5 | Promoter (1-2kb) |
| chr1: 6473356 | PLEKHG5 | Promoter (1-2kb) |
| chr1: 6473407 | PLEKHG5 | Promoter (1-2kb) |
| chr1: 6473425 | PLEKHG5 | Promoter (1-2kb) |
| chr1: 6473439 | PLEKHG5 | Promoter (1-2kb) |
| chr1: 6473441 | PLEKHG5 | Promoter (1-2kb) |
| chr1: 6473442 | PLEKHG5 | Promoter (1-2kb) |
| chr1: 7574644 | CAMTA1 | Intron (ENST00000303635.12/23261, intron 6 of 22) |
| chr1: 7574659 | CAMTA1 | Intron (ENST00000303635.12/23261, intron 6 of 22) |
| chr1: 7574678 | CAMTA1 | Intron (ENST00000303635.12/23261, intron 6 of 22) |
| chr1: 10658845 | CASZ1 | Promoter (2-3kb) |
| chr1: 10658877 | CASZ1 | Promoter (2-3kb) |
| chr1: 10665296 | CASZ1 | Exon (ENST00000377022.8/54897, exon 5 of 21) |
| chr1: 10665323 | CASZ1 | Exon (ENST00000377022.8/54897, exon 5 of 21) |
| chr1: 10665324 | CASZ1 | Exon (ENST00000377022.8/54897, exon 5 of 21) |
| chr1: 10665334 | CASZ1 | Exon (ENST00000377022.8/54897, exon 5 of 21) |
| chr1: 10665335 | CASZ1 | Exon (ENST00000377022.8/54897, exon 5 of 21) |
| chr1: 10665351 | CASZ1 | Exon (ENST00000377022.8/54897, exon 5 of 21) |
| chr1: 10665356 | CASZ1 | Exon (ENST00000377022.8/54897, exon 5 of 21) |
| chr1: 10665391 | CASZ1 | Exon (ENST00000377022.8/54897, exon 5 of 21) |
| chr1: 10665392 | CASZ1 | Exon (ENST00000377022.8/54897, exon 5 of 21) |
| chr1: 10665448 | CASZ1 | Exon (ENST00000377022.8/54897, exon 5 of 21) |
| chr1: 10665469 | CASZ1 | Exon (ENST00000377022.8/54897, exon 5 of 21) |
| chr1: 10665515 | CASZ1 | Exon (ENST00000377022.8/54897, exon 5 of 21) |
| chr1: 10665523 | CASZ1 | Exon (ENST00000377022.8/54897, exon 5 of 21) |
| chr1: 10665531 | CASZ1 | Exon (ENST00000377022.8/54897, exon 5 of 21) |
| chr1: 10665547 | CASZ1 | Exon (ENST00000377022.8/54897, exon 5 of 21) |
| chr1: 10744882 | CASZ1 | Intron (ENST00000377022.8/54897, intron 2 of 20) |
| chr1: 10744889 | CASZ1 | Intron (ENST00000377022.8/54897, intron 2 of 20) |
| chr1: 10744895 | CASZ1 | Intron (ENST00000377022.8/54897, intron 2 of 20) |
| chr1: 10744915 | CASZ1 | Intron (ENST00000377022.8/54897, intron 2 of 20) |
| chr1: 10744948 | CASZ1 | Intron (ENST00000377022.8/54897, intron 2 of 20) |
| chr1: 10814011 | CASZ1 | Distal Intergenic |
| chr1: 10958789 | C1orf127 | Intron (ENST00000418570.6/148345, intron 1 of 7) |
| chr1: 10958790 | C1orf127 | Intron (ENST00000418570.6/148345, intron 1 of 7) |
| chr1: 10958875 | C1orf127 | Intron (ENST00000418570.6/148345, intron 1 of 7) |
| chr1: 10958941 | C1orf127 | Intron (ENST00000418570.6/148345, intron 1 of 7) |
| chr1: 13901406 | KAZN | Intron (ENST00000636564.1/ENST00000636564.1, intron 1 of 2) |
| chr1: 14420671 | KAZN-AS1 | Promoter (<=1kb) |
| chr1: 14420679 | KAZN-AS1 | Promoter (<=1kb) |
| chr1: 15467505 | CELA2B | Promoter (1-2kb) |
| chr1: 15467507 | CELA2B | Promoter (1-2kb) |
| chr1: 15467524 | CELA2B | Promoter (1-2kb) |
| chr1: 24322601 | GRHL3 | Promoter (<=1kb) |
| chr1: 24322603 | GRHL3 | Promoter (<=1kb) |
| chr1: 24322614 | GRHL3 | Promoter (<=1kb) |
| chr1: 24322620 | GRHL3 | Promoter (<=1kb) |
| chr1: 24322636 | GRHL3 | Promoter (<=1kb) |
| chr1: 24642007 | SRRM1 | Promoter (<=1kb) |
| chr1: 24642020 | SRRM1 | Promoter (<=1kb) |
| chr1: 24642021 | SRRM1 | Promoter (<=1kb) |
| chr1: 24642029 | SRRM1 | Promoter (<=1kb) |
| chr1: 24642030 | SRRM1 | Promoter (<=1kb) |
| chr1: 24642036 | SRRM1 | Promoter (<=1kb) |
| chr1: 24642037 | SRRM1 | Promoter (<=1kb) |
| chr1: 24642042 | SRRM1 | Promoter (<=1kb) |
| chr1: 24642043 | SRRM1 | Promoter (<=1kb) |
| chr1: 26863494 | SFN | Promoter (<=1kb) |
| chr1: 26863500 | SFN | Promoter (<=1kb) |
| chr1: 26863501 | SFN | Promoter (<=1kb) |
| chr1: 26863506 | SFN | Promoter (<=1kb) |
| chr1: 26863507 | SFN | Promoter (<=1kb) |
| chr1: 26863546 | SFN | Promoter (<=1kb) |
| chr1: 26863597 | SFN | Promoter (<=1kb) |
| chr1: 26863598 | SFN | Promoter (<=1kb) |
| chr1: 29259879 | PTPRU | Promoter (<=1kb) |
| chr1: 29259884 | PTPRU | Promoter (<=1kb) |
| chr1: 31176418 | NKAIN1 | Distal Intergenic |
| chr1: 31176426 | NKAIN1 | Distal Intergenic |
| chr1: 31176444 | NKAIN1 | Distal Intergenic |
| chr1: 31714647 | COL16A1 | Distal Intergenic |
| chr1: 31714655 | COL16A1 | Distal Intergenic |
| chr1: 31714656 | COL16A1 | Distal Intergenic |
| chr1: 31714688 | COL16A1 | Distal Intergenic |
| chr1: 31714693 | COL16A1 | Distal Intergenic |
| chr1: 31714706 | COL16A1 | Distal Intergenic |
| chr1: 34865995 | SMIM12 | 3′ UTR |
| chr1: 34866000 | SMIM12 | 3′ UTR |
| chr1: 34866002 | SMIM12 | 3′ UTR |
| chr1: 34866017 | SMIM12 | 3′ UTR |
| chr1: 34866020 | SMIM12 | 3′ UTR |
| chr1: 34866103 | SMIM12 | Exon (ENST00000235180.4/58512, exon 10 of 10) |
| chr1: 34866118 | SMIM12 | Exon (ENST00000235180.4/58512, exon 10 of 10) |
| chr1: 34866127 | SMIM12 | Exon (ENST00000235180.4/58512, exon 10 of 10) |
| chr1: 36161139 | MAP7D1 | Intron (ENST00000429533.6/55700, intron 1 of 5) |
| chr1: 36161140 | MAP7D1 | Intron (ENST00000429533.6/55700, intron 1 of 5) |
| chr1: 38559563 | LINC01685 | Distal Intergenic |
| chr1: 38559572 | LINC01685 | Distal Intergenic |
| chr1: 38559577 | LINC01685 | Distal Intergenic |
| chr1: 40802458 | KCNQ4 | Intron (ENST00000347132.10/9132, intron 1 of 13) |
| chr1: 40802464 | KCNQ4 | Intron (ENST00000347132.10/9132, intron 1 of 13) |
| chr1: 40802488 | KCNQ4 | Intron (ENST00000347132.10/9132, intron 1 of 13) |
| chr1: 44576768 | MIR5584 | Intron (ENST00000355387.6/55182, intron 2 of 14) |
| chr1: 44588236 | RNF220 | Intron (ENST00000355387.6/55182, intron 2 of 14) |
| chr1: 44588256 | RNF220 | Intron (ENST00000355387.6/55182, intron 2 of 14) |
| chr1: 44603252 | RNF220 | Intron (ENST00000355387.6/55182, intron 2 of 14) |
| chr1: 44603267 | RNF220 | Intron (ENST00000355387.6/55182, intron 2 of 14) |
| chr1: 47230856 | TAL1 | Promoter (<=1kb) |
| chr1: 47230862 | TAL1 | Promoter (<=1kb) |
| chr1: 47230868 | TAL1 | Promoter (<=1kb) |
| chr1: 47230874 | TAL1 | Promoter (<=1kb) |
| chr1: 47230877 | TAL1 | Promoter (<=1kb) |
| chr1: 47230894 | TAL1 | Promoter (<=1kb) |
| chr1: 50416355 | DMRTA2 | Downstream (1-2kb) |
| chr1: 50416362 | DMRTA2 | Downstream (1-2kb) |
| chr1: 50416430 | DMRTA2 | Downstream (1-2kb) |
| chr1: 50416561 | DMRTA2 | Downstream (<1kb) |
| chr1: 50416568 | DMRTA2 | Downstream (<1kb) |
| chr1: 54980471 | TMEM61 | Promoter (<=1kb) |
| chr1: 54980476 | TMEM61 | Promoter (<=1kb) |
| chr1: 54980498 | TMEM61 | Promoter (<=1kb) |
| chr1: 54980503 | TMEM61 | Promoter (<=1kb) |
| chr1: 54980505 | TMEM61 | Promoter (<=1kb) |
| chr1: 54980513 | TMEM61 | Promoter (<=1kb) |
| chr1: 54980533 | TMEM61 | Promoter (<=1kb) |
| chr1: 54980535 | TMEM61 | Promoter (<=1kb) |
| chr1: 54980543 | TMEM61 | Promoter (<=1kb) |
| chr1: 54980550 | TMEM61 | Promoter (<=1kb) |
| chr1: 54980553 | TMEM61 | Promoter (<=1kb) |
| chr1: 54980555 | TMEM61 | Promoter (<=1kb) |
| chr1: 54980714 | TMEM61 | Promoter (<=1kb) |
| chr1: 54980716 | TMEM61 | Promoter (<=1kb) |
| chr1: 54980728 | TMEM61 | Promoter (<=1kb) |
| chr1: 54980735 | TMEM61 | Promoter (<=1kb) |
| chr1: 54980736 | TMEM61 | Promoter (<=1kb) |
| chr1: 54980752 | TMEM61 | Promoter (<=1kb) |
| chr1: 54980753 | TMEM61 | Promoter (<=1kb) |
| chr1: 54980754 | TMEM61 | Promoter (<=1kb) |
| chr1: 54980755 | TMEM61 | Promoter (<=1kb) |
| chr1: 54980762 | TMEM61 | Promoter (<=1kb) |
| chr1: 54980764 | TMEM61 | Promoter (<=1kb) |
| chr1: 54980773 | TMEM61 | Promoter (<=1kb) |
| chr1: 54980778 | TMEM61 | Promoter (<=1kb) |
| chr1: 54980782 | TMEM61 | Promoter (<=1kb) |
| chr1: 61054727 | NFIA | Promoter (<=1kb) |
| chr1: 61054728 | NFIA | Promoter (<=1kb) |
| chr1: 61054749 | NFIA | Promoter (<=1kb) |
| chr1: 61054752 | NFIA | Promoter (<=1kb) |
| chr1: 61054757 | NFIA | Promoter (<=1kb) |
| chr1: 63330395 | MIR6068 | Distal Intergenic |
| chr1: 63330398 | MIR6068 | Distal Intergenic |
| chr1: 63330404 | MIR6068 | Distal Intergenic |
| chr1: 63330412 | MIR6068 | Distal Intergenic |
| chr1: 63330419 | MIR6068 | Distal Intergenic |
| chr1: 63330421 | MIR6068 | Distal Intergenic |
| chr1: 75797204 | MSH4 | Promoter (<=1kb) |
| chr1: 75797205 | MSH4 | Promoter (<=1kb) |
| chr1: 75797206 | MSH4 | Promoter (<=1kb) |
| chr1: 75797207 | MSH4 | Promoter (<=1kb) |
| chr1: 90836081 | LINC02609 | Promoter (<=1kb) |
| chr1: 90836087 | LINC02609 | Promoter (<=1kb) |
| chr1: 90836091 | LINC02609 | Promoter (<=1kb) |
| chr1: 90836094 | LINC02609 | Promoter (<=1kb) |
| chr1: 90836113 | LINC02609 | Promoter (<=1kb) |
| chr1: 90836117 | LINC02609 | Promoter (<=1kb) |
| chr1: 101256143 | S1PR1 | Distal Intergenic |
| chr1: 108675439 | HENMT1 | Distal Intergenic |
| chr1: 108675464 | HENMT1 | Distal Intergenic |
| chr1: 109274863 | CELSR2 | 3′ UTR |
| chr1: 109274868 | CELSR2 | 3′ UTR |
| chr1: 109274870 | CELSR2 | 3′ UTR |
| chr1: 111961251 | KCND3 | Intron (ENST00000369697.5/3752, intron 1 of 5) |
| chr1: 113422496 | MAGI3 | Intron (ENST00000307546.14/260425, intron 1 of 20) |
| chr1: 116168253 | MAB21L3 | Distal Intergenic |
| chr1: 116168278 | MAB21L3 | Distal Intergenic |
| chr1: 116168454 | MAB21L3 | Distal Intergenic |
| chr1: 116168455 | MAB21L3 | Distal Intergenic |
| chr1: 116168456 | MAB21L3 | Distal Intergenic |
| chr1: 116168457 | MAB21L3 | Distal Intergenic |
| chr1: 116168477 | MAB21L3 | Distal Intergenic |
| chr1: 116168478 | MAB21L3 | Distal Intergenic |
| chr1: 143654112 | RNVU1-17 | Distal Intergenic |
| chr1: 143654118 | RNVU1-17 | Distal Intergenic |
| chr1: 143691944 | RNVU1-17 | Distal Intergenic |
| chr1: 143691957 | RNVU1-17 | Distal Intergenic |
| chr1: 149813047 | H3C13 | Promoter (<=1kb) |
| chr1: 149813051 | H3C13 | Promoter (<=1kb) |
| chr1: 150716911 | HORMAD1 | Intron (ENST00000368995.8/84072, intron 3 of 12) |
| chr1: 151473479 | POGZ | Distal Intergenic |
| chr1: 151473485 | POGZ | Distal Intergenic |
| chr1: 151473487 | POGZ | Distal Intergenic |
| chr1: 151473495 | POGZ | Distal Intergenic |
| chr1: 151473497 | POGZ | Distal Intergenic |
| chr1: 151473500 | POGZ | Distal Intergenic |
| chr1: 151473502 | POGZ | Distal Intergenic |
| chr1: 151473584 | POGZ | Distal Intergenic |
| chr1: 151473591 | POGZ | Distal Intergenic |
| chr1: 151473605 | POGZ | Distal Intergenic |
| chr1: 151473609 | POGZ | Distal Intergenic |
| chr1: 151769408 | OAZ3 | Promoter (2-3kb) |
| chr1: 154502739 | TDRD10 | Promoter (<=1kb) |
| chr1: 154502777 | TDRD10 | Promoter (<=1kb) |
| chr1: 154502794 | TDRD10 | Promoter (<=1kb) |
| chr1: 154502799 | TDRD10 | Promoter (<=1kb) |
| chr1: 154502809 | TDRD10 | Promoter (<=1kb) |
| chr1: 154502820 | TDRD10 | Promoter (<=1kb) |
| chr1: 154502824 | TDRD10 | Promoter (<=1kb) |
| chr1: 156077503 | MEX3A | Exon (ENST00000532414.3/92312, exon 2 of 2) |
| chr1: 160371182 | NHLH1 | 3′ UTR |
| chr1: 160371202 | NHLH1 | 3′ UTR |
| chr1: 164792823 | PBX1 | Promoter (<=1kb) |
| chr1: 164792824 | PBX1 | Promoter (<=1kb) |
| chr1: 170661589 | PRRX1 | Promoter (1-2kb) |
| chr1: 170661593 | PRRX1 | Promoter (1-2kb) |
| chr1: 170661600 | PRRX1 | Promoter (1-2kb) |
| chr1: 170661602 | PRRX1 | Promoter (1-2kb) |
| chr1: 170661603 | PRRX1 | Promoter (1-2kb) |
| chr1: 170661605 | PRRX1 | Promoter (1-2kb) |
| chr1: 170661606 | PRRX1 | Promoter (1-2kb) |
| chr1: 170661614 | PRRX1 | Promoter (1-2kb) |
| chr1: 175907228 | LINC01657 | Intron (ENST00000426575.2/ENST00000426575.2, intron 2 of 2) |
| chr1: 175907235 | LINC01657 | Intron (ENST00000426575.2/ENST00000426575.2, intron 2 of 2) |
| chr1: 175907238 | LINC01657 | Intron (ENST00000426575.2/ENST00000426575.2, intron 2 of 2) |
| chr1: 179138847 | ABL2 | Intron (ENST00000367623.8/27, intron 1 of 10) |
| chr1: 179138848 | ABL2 | Intron (ENST00000367623.8/27, intron 1 of 10) |
| chr1: 179138917 | ABL2 | Intron (ENST00000367623.8/27, intron 1 of 10) |
| chr1: 179138918 | ABL2 | Intron (ENST00000367623.8/27, intron 1 of 10) |
| chr1: 179138924 | ABL2 | Intron (ENST00000367623.8/27, intron 1 of 10) |
| chr1: 179138925 | ABL2 | Intron (ENST00000367623.8/27, intron 1 of 10) |
| chr1: 184942664 | NIBAN1 | Intron (ENST00000367511.4/116496, intron 1 of 13) |
| chr1: 201127109 | ASCL5 | 5′ UTR |
| chr1: 201127206 | ASCL5 | Distal Intergenic |
| chr1: 202010615 | ELF3 | Promoter (<=1kb) |
| chr1: 202010620 | ELF3 | Promoter (<=1kb) |
| chr1: 202010636 | ELF3 | Promoter (<=1kb) |
| chr1: 202010637 | ELF3 | Promoter (<=1kb) |
| chr1: 202010646 | ELF3 | Promoter (<=1kb) |
| chr1: 202010647 | ELF3 | Promoter (<=1kb) |
| chr1: 202010666 | ELF3 | Promoter (<=1kb) |
| chr1: 202010673 | ELF3 | Promoter (<=1kb) |
| chr1: 205343546 | KLHDC8A | Promoter (<=1kb) |
| chr1: 205343560 | KLHDC8A | Promoter (<=1kb) |
| chr1: 205343567 | KLHDC8A | Promoter (<=1kb) |
| chr1: 205343712 | KLHDC8A | Promoter (<=1kb) |
| chr1: 205343713 | KLHDC8A | Promoter (<=1kb) |
| chr1: 205343716 | KLHDC8A | Promoter (<=1kb) |
| chr1: 205343717 | KLHDC8A | Promoter (<=1kb) |
| chr1: 205343751 | KLHDC8A | Promoter (<=1kb) |
| chr1: 205343752 | KLHDC8A | Promoter (<=1kb) |
| chr1: 205343758 | KLHDC8A | Promoter (<=1kb) |
| chr1: 205343759 | KLHDC8A | Promoter (<=1kb) |
| chr1: 205343768 | KLHDC8A | Promoter (<=1kb) |
| chr1: 205343769 | KLHDC8A | Promoter (<=1kb) |
| chr1: 205343835 | KLHDC8A | Promoter (<=1kb) |
| chr1: 205343837 | KLHDC8A | Promoter (<=1kb) |
| chr1: 205343846 | KLHDC8A | Promoter (<=1kb) |
| chr1: 205343853 | KLHDC8A | Promoter (<=1kb) |
| chr1: 205343864 | KLHDC8A | Promoter (<=1kb) |
| chr1: 230839344 | C1orf198 | 3′ UTR |
| chr1: 230839348 | C1orf198 | 3′ UTR |
| chr1: 230839361 | C1orf198 | 3′ UTR |
| chr1: 230839364 | C1orf198 | 3′ UTR |
| chr1: 245234766 | KIF26B-AS1 | Promoter (<=1kb) |
| chr1: 245234785 | KIF26B-AS1 | Promoter (<=1kb) |
| chr1: 245234788 | KIF26B-AS1 | Promoter (<=1kb) |
| chr1: 245234801 | KIF26B-AS1 | Promoter (<=1kb) |
| chr10: 42898212 | LINC02623 | Distal Intergenic |
| chr10: 42898220 | LINC02623 | Distal Intergenic |
| chr10: 42898225 | LINC02623 | Distal Intergenic |
| chr10: 75398841 | ZNF503 | Promoter (2-3kb) |
| chr10: 75398847 | ZNF503 | Promoter (2-3kb) |
| chr10: 75398854 | ZNF503 | Promoter (2-3kb) |
| chr10: 75398863 | ZNF503 | Promoter (2-3kb) |
| chr10: 100741985 | PAX2 | Intron (ENST00000553492.5/5076, intron 1 of 3) |
| chr10: 100741996 | PAX2 | Intron (ENST00000553492.5/5076, intron 1 of 3) |
| chr10: 100741998 | PAX2 | Intron (ENST00000553492.5/5076, intron 1 of 3) |
| chr10: 100742013 | PAX2 | Intron (ENST00000553492.5/5076, intron 1 of 3) |
| chr10: 100742015 | PAX2 | Intron (ENST00000553492.5/5076, intron 1 of 3) |
| chr10: 100742025 | PAX2 | Intron (ENST00000553492.5/5076, intron 1 of 3) |
| chr10: 100742026 | PAX2 | Intron (ENST00000553492.5/5076, intron 1 of 3) |
| chr10: 100742045 | PAX2 | Intron (ENST00000553492.5/5076, intron 1 of 3) |
| chr10: 102485644 | ACTR1A | Promoter (1-2kb) |
| chr10: 102485662 | ACTR1A | Promoter (1-2kb) |
| chr10: 102485674 | ACTR1A | Promoter (1-2kb) |
| chr10: 102485682 | ACTR1A | Promoter (1-2kb) |
| chr10: 102485696 | ACTR1A | Promoter (1-2kb) |
| chr10: 102485710 | ACTR1A | Promoter (1-2kb) |
| chr10: 116670071 | C10orf82 | Promoter (<=1kb) |
| chr10: 116670073 | C10orf82 | Promoter (<=1kb) |
| chr10: 117535066 | EMX2OS | Intron (ENST00000551288.5/196047, intron 2 of 3) |
| chr10: 117535091 | EMX2OS | Intron (ENST00000551288.5/196047, intron 2 of 3) |
| chr10: 117535092 | EMX2OS | Intron (ENST00000551288.5/196047, intron 2 of 3) |
| chr10: 117535329 | EMX2OS | Intron (ENST00000551288.5/196047, intron 2 of 3) |
| chr10: 117535336 | EMX2OS | Intron (ENST00000551288.5/196047, intron 2 of 3) |
| chr10: 117535352 | EMX2OS | Intron (ENST00000551288.5/196047, intron 2 of 3) |
| chr10: 117535357 | EMX2OS | Intron (ENST00000551288.5/196047, intron 2 of 3) |
| chr10: 117535366 | EMX2OS | Intron (ENST00000551288.5/196047, intron 2 of 3) |
| chr10: 117535389 | EMX2OS | Intron (ENST00000551288.5/196047, intron 2 of 3) |
| chr10: 117535393 | EMX2OS | Intron (ENST00000551288.5/196047, intron 2 of 3) |
| chr10: 117535397 | EMX2OS | Intron (ENST00000551288.5/196047, intron 2 of 3) |
| chr10: 117535399 | EMX2OS | Intron (ENST00000551288.5/196047, intron 2 of 3) |
| chr10: 119541831 | RGS10 | Promoter (<=1kb) |
| chr10: 119541833 | RGS10 | Promoter (<=1kb) |
| chr10: 127424596 | NPS | Intron (ENST00000280333.9/1793, intron 46 of 51) |
| chr10: 127424611 | NPS | Intron (ENST00000280333.9/1793, intron 46 of 51) |
| chr10: 127424672 | NPS | Intron (ENST00000280333.9/1793, intron 46 of 51) |
| chr10: 127424674 | NPS | Intron (ENST00000280333.9/1793, intron 46 of 51) |
| chr10: 129580062 | MGMT | Intron (ENST00000306010.8/4255, intron 2 of 4) |
| chr10: 129580113 | MGMT | Intron (ENST00000306010.8/4255, intron 2 of 4) |
| chr10: 132065569 | JAKMIP3 | Promoter (<=1kb) |
| chr10: 132065581 | JAKMIP3 | Promoter (<=1kb) |
| chr10: 132065585 | JAKMIP3 | Promoter (<=1kb) |
| chr10: 132065597 | JAKMIP3 | Promoter (<=1kb) |
| chr10: 132065608 | JAKMIP3 | Promoter (<=1kb) |
| chr10: 132295081 | STK32C | Intron (ENST00000368620.2/282974, intron 1 of 3) |
| chr10: 132295110 | STK32C | Intron (ENST00000368620.2/282974, intron 1 of 3) |
| chr10: 132295119 | STK32C | Intron (ENST00000368620.2/282974, intron 1 of 3) |
| chr10: 132295120 | STK32C | Intron (ENST00000368620.2/282974, intron 1 of 3) |
| chr10: 132685767 | INPP5A | Intron (ENST00000423490.5/3632, intron 2 of 5) |
| chr10: 132685769 | INPP5A | Intron (ENST00000423490.5/3632, intron 2 of 5) |
| chr10: 132685807 | INPP5A | Intron (ENST00000423490.5/3632, intron 2 of 5) |
| chr10: 132785047 | NKX6-2 | Promoter (1-2kb) |
| chr10: 132785054 | NKX6-2 | Promoter (1-2kb) |
| chr10: 132785056 | NKX6-2 | Promoter (1-2kb) |
| chr10: 132785060 | NKX6-2 | Promoter (1-2kb) |
| chr11: 2211321 | MIR4686 | Distal Intergenic |
| chr11: 2211334 | MIR4686 | Distal Intergenic |
| chr11: 2575187 | KCNQ1 | Intron (ENST00000646564.1/3784, intron 2 of 10) |
| chr11: 2575220 | KCNQ1 | Intron (ENST00000646564.1/3784, intron 2 of 10) |
| chr11: 2575224 | KCNQ1 | Intron (ENST00000646564.1/3784, intron 2 of 10) |
| chr11: 2575231 | KCNQ1 | Intron (ENST00000646564.1/3784, intron 2 of 10) |
| chr11: 14091669 | SPON1 | Intron (ENST00000576479.4/10418, intron 5 of 15) |
| chr11: 14091678 | SPON1 | Intron (ENST00000576479.4/10418, intron 5 of 15) |
| chr11: 14091682 | SPON1 | Intron (ENST00000576479.4/10418, intron 5 of 15) |
| chr11: 32333891 | WT1 | Intron (ENST00000525689.2/ENST00000525689.2, intron 1 of 1) |
| chr11: 32333902 | WT1 | Intron (ENST00000525689.2/ENST00000525689.2, intron 1 of 1) |
| chr11: 32333905 | WT1 | Intron (ENST00000525689.2/ENST00000525689.2, intron 1 of 1) |
| chr11: 32434782 | WT1 | Promoter (<=1kb) |
| chr11: 32434794 | WT1 | Promoter (<=1kb) |
| chr11: 32434805 | WT1 | Promoter (<=1kb) |
| chr11: 33406359 | KIAA1549L | Intron (ENST00000658780.1/25758, intron 1 of 20) |
| chr11: 33406375 | KIAA1549L | Intron (ENST00000658780.1/25758, intron 1 of 20) |
| chr11: 33406381 | KIAA1549L | Intron (ENST00000658780.1/25758, intron 1 of 20) |
| chr11: 35420544 | SLC1A2 | Promoter (<=1kb) |
| chr11: 35420552 | SLC1A2 | Promoter (<=1kb) |
| chr11: 35420553 | SLC1A2 | Promoter (<=1kb) |
| chr11: 35420574 | SLC1A2 | Promoter (<=1kb) |
| chr11: 35420575 | SLC1A2 | Promoter (<=1kb) |
| chr11: 35420583 | SLC1A2 | Promoter (<=1kb) |
| chr11: 43581402 | MIR129-2 | Promoter (<=1kb) |
| chr11: 43581404 | MIR129-2 | Promoter (<=1kb) |
| chr11: 43581416 | MIR129-2 | Promoter (<=1kb) |
| chr11: 46385382 | MDK | Exon (ENST00000433765.3/ENST00000433765.3, exon 1 of 1) |
| chr11: 46385392 | MDK | Exon (ENST00000433765.3/ENST00000433765.3, exon 1 of 1) |
| chr11: 46385401 | MDK | Exon (ENST00000433765.3/ENST00000433765.3, exon 1 of 1) |
| chr11: 46385413 | MDK | Exon (ENST00000433765.3/ENST00000433765.3, exon 1 of 1) |
| chr11: 46385417 | MDK | Exon (ENST00000433765.3/ENST00000433765.3, exon 1 of 1) |
| chr11: 46385418 | MDK | Exon (ENST00000433765.3/ENST00000433765.3, exon 1 of 1) |
| chr11: 46385426 | MDK | Exon (ENST00000433765.3/ENST00000433765.3, exon 1 of 1) |
| chr11: 46385427 | MDK | Exon (ENST00000433765.3/ENST00000433765.3, exon 1 of 1) |
| chr11: 46385435 | MDK | Exon (ENST00000433765.3/ENST00000433765.3, exon 1 of 1) |
| chr11: 46385442 | MDK | Exon (ENST00000433765.3/ENST00000433765.3, exon 1 of 1) |
| chr11: 46385449 | MDK | Exon (ENST00000433765.3/ENST00000433765.3, exon 1 of 1) |
| chr11: 46385456 | MDK | Exon (ENST00000433765.3/ENST00000433765.3, exon 1 of 1) |
| chr11: 59565879 | MIR3162 | Exon (ENST00000661408.1/ENST00000661408.1, exon 1 of 8) |
| chr11: 59565903 | MIR3162 | Exon (ENST00000661408.1/ENST00000661408.1, exon 1 of 8) |
| chr11: 59565904 | MIR3162 | Exon (ENST00000661408.1/ENST00000661408.1, exon 1 of 8) |
| chr11: 59565907 | MIR3162 | Exon (ENST00000661408.1/ENST00000661408.1, exon 1 of 8) |
| chr11: 59565908 | MIR3162 | Exon (ENST00000661408.1/ENST00000661408.1, exon 1 of 8) |
| chr11: 59565910 | MIR3162 | Exon (ENST00000661408.1/ENST00000661408.1, exon 1 of 8) |
| chr11: 59565911 | MIR3162 | Exon (ENST00000661408.1/ENST00000661408.1, exon 1 of 8) |
| chr11: 59565914 | MIR3162 | Exon (ENST00000661408.1/ENST00000661408.1, exon 1 of 8) |
| chr11: 59565915 | MIR3162 | Exon (ENST00000661408.1/ENST00000661408.1, exon 1 of 8) |
| chr11: 59565921 | MIR3162 | Exon (ENST00000661408.1/ENST00000661408.1, exon 1 of 8) |
| chr11: 59565974 | MIR3162 | Exon (ENST00000661408.1/ENST00000661408.1, exon 1 of 8) |
| chr11: 59565976 | MIR3162 | Exon (ENST00000661408.1/ENST00000661408.1, exon 1 of 8) |
| chr11: 59565991 | MIR3162 | Exon (ENST00000661408.1/ENST00000661408.1, exon 1 of 8) |
| chr11: 59565997 | MIR3162 | Exon (ENST00000661408.1/ENST00000661408.1, exon 1 of 8) |
| chr11: 59566000 | MIR3162 | Exon (ENST00000661408.1/ENST00000661408.1, exon 1 of 8) |
| chr11: 59566007 | MIR3162 | Exon (ENST00000661408.1/ENST00000661408.1, exon 1 of 8) |
| chr11: 59566091 | MIR3162 | Exon (ENST00000661408.1/ENST00000661408.1, exon 1 of 8) |
| chr11: 60852858 | PTGDR2 | Exon (ENST00000332539.5/11251, exon 2 of 2) |
| chr11: 60852859 | PTGDR2 | Exon (ENST00000332539.5/11251, exon 2 of 2) |
| chr11: 60852871 | PTGDR2 | Exon (ENST00000332539.5/11251, exon 2 of 2) |
| chr11: 60852873 | PTGDR2 | Exon (ENST00000332539.5/11251, exon 2 of 2) |
| chr11: 60852884 | PTGDR2 | Exon (ENST00000332539.5/11251, exon 2 of 2) |
| chr11: 60852888 | PTGDR2 | Exon (ENST00000332539.5/11251, exon 2 of 2) |
| chr11: 60852903 | PTGDR2 | Exon (ENST00000332539.5/11251, exon 2 of 2) |
| chr11: 60852907 | PTGDR2 | Exon (ENST00000332539.5/11251, exon 2 of 2) |
| chr11: 60852911 | PTGDR2 | Exon (ENST00000332539.5/11251, exon 2 of 2) |
| chr11: 60852913 | PTGDR2 | Exon (ENST00000332539.5/11251, exon 2 of 2) |
| chr11: 64081931 | FLRT1 | Exon (ENST00000544948.1/ENST00000544948.1, exon 1 of 3) |
| chr11: 64081938 | FLRT1 | Exon (ENST00000544948.1/ENST00000544948.1, exon 1 of 3) |
| chr11: 64081940 | FLRT1 | Exon (ENST00000544948.1/ENST00000544948.1, exon 1 of 3) |
| chr11: 64082013 | FLRT1 | Exon (ENST00000544948.1/ENST00000544948.1, exon 1 of 3) |
| chr11: 64082017 | FLRT1 | Exon (ENST00000544948.1/ENST00000544948.1, exon 1 of 3) |
| chr11: 64082026 | FLRT1 | Exon (ENST00000544948.1/ENST00000544948.1, exon 1 of 3) |
| chr11: 64082135 | FLRT1 | Intron (ENST00000545464.5/28992, intron 3 of 3) |
| chr11: 64082193 | FLRT1 | Intron (ENST00000545464.5/28992, intron 3 of 3) |
| chr11: 64082195 | FLRT1 | Intron (ENST00000545464.5/28992, intron 3 of 3) |
| chr11: 64353134 | CCDC88B | Promoter (<=1kb) |
| chr11: 64353136 | CCDC88B | Promoter (<=1kb) |
| chr11: 64353151 | CCDC88B | Promoter (<=1kb) |
| chr11: 64353185 | CCDC88B | Promoter (<=1kb) |
| chr11: 64353187 | CCDC88B | Promoter (<=1kb) |
| chr11: 64353214 | CCDC88B | Promoter (<=1kb) |
| chr11: 65551191 | LTBP3 | Promoter (<=1kb) |
| chr11: 65551199 | LTBP3 | Promoter (<=1kb) |
| chr11: 65551205 | LTBP3 | Promoter (<=1kb) |
| chr11: 65551212 | LTBP3 | Promoter (<=1kb) |
| chr11: 66316102 | CD248 | Promoter (<=1kb) |
| chr11: 66316103 | CD248 | Promoter (<=1kb) |
| chr11: 66316126 | CD248 | Promoter (<=1kb) |
| chr11: 68039070 | TCIRG1 | Promoter (<=1kb) |
| chr11: 68039077 | TCIRG1 | Promoter (<=1kb) |
| chr11: 68039092 | TCIRG1 | Promoter (<=1kb) |
| chr11: 68039098 | TCIRG1 | Promoter (<=1kb) |
| chr11: 68039100 | TCIRG1 | Promoter (<=1kb) |
| chr11: 68039144 | TCIRG1 | Promoter (<=1kb) |
| chr11: 68946886 | IGHMBP2 | Distal Intergenic |
| chr11: 68946898 | IGHMBP2 | Distal Intergenic |
| chr11: 69654991 | CCND1 | Intron (ENST00000542515.5/ENST00000542515.5, intron 1 of 2) |
| chr11: 69655008 | CCND1 | Intron (ENST00000542515.5/ENST00000542515.5, intron 1 of 2) |
| chr11: 69655050 | CCND1 | Intron (ENST00000542515.5/ENST00000542515.5, intron 1 of 2) |
| chr11: 69655053 | CCND1 | Intron (ENST00000542515.5/ENST00000542515.5, intron 1 of 2) |
| chr11: 72106024 | LRTOMT | Promoter (<=1kb) |
| chr11: 72106036 | LRTOMT | Promoter (<=1kb) |
| chr11: 72106040 | LRTOMT | Promoter (<=1kb) |
| chr11: 72106051 | LRTOMT | Promoter (<=1kb) |
| chr11: 72106087 | LRTOMT | Promoter (<=1kb) |
| chr11: 72106094 | LRTOMT | Promoter (<=1kb) |
| chr11: 72106108 | LRTOMT | Promoter (<=1kb) |
| chr11: 76292339 | THAP12 | Distal Intergenic |
| chr11: 76292340 | THAP12 | Distal Intergenic |
| chr11: 76292356 | THAP12 | Distal Intergenic |
| chr11: 76292368 | THAP12 | Distal Intergenic |
| chr11: 76292392 | THAP12 | Distal Intergenic |
| chr11: 76292393 | THAP12 | Distal Intergenic |
| chr11: 76292397 | THAP12 | Distal Intergenic |
| chr11: 76292398 | THAP12 | Distal Intergenic |
| chr11: 95151013 | LOC101929295 | Promoter (<=1kb) |
| chr11: 95151031 | LOC101929295 | Promoter (<=1kb) |
| chr11: 95151034 | LOC101929295 | Promoter (<=1kb) |
| chr11: 95232446 | SESN3 | Promoter (<=1kb) |
| chr11: 95232447 | SESN3 | Promoter (<=1kb) |
| chr11: 117820155 | DSCAML1 | Promoter (2-3kb) |
| chr11: 117820165 | DSCAML1 | Promoter (2-3kb) |
| chr11: 125099290 | TMEM218 | Intron (ENST00000531909.5/219854, intron 4 of 4) |
| chr11: 125099307 | TMEM218 | Intron (ENST00000531909.5/219854, intron 4 of 4) |
| chr11: 125099311 | TMEM218 | Intron (ENST00000531909.5/219854, intron 4 of 4) |
| chr11: 125099316 | TMEM218 | Intron (ENST00000531909.5/219854, intron 4 of 4) |
| chr11: 128860025 | KCNJ1 | Intron (ENST00000392665.6/3758, intron 1 of 1) |
| chr11: 128860035 | KCNJ1 | Intron (ENST00000392665.6/3758, intron 1 of 1) |
| chr11: 128860042 | KCNJ1 | Intron (ENST00000392665.6/3758, intron 1 of 1) |
| chr11: 128860066 | KCNJ1 | Intron (ENST00000392665.6/3758, intron 1 of 1) |
| chr11: 129316134 | ARHGAP32 | Distal Intergenic |
| chr11: 129316137 | ARHGAP32 | Distal Intergenic |
| chr11: 130179878 | ST14 | Intron (ENST00000278742.6/6768, intron 1 of 18) |
| chr11: 130179900 | ST14 | Intron (ENST00000278742.6/6768, intron 1 of 18) |
| chr11: 130209380 | ST14 | Intron (ENST00000278742.6/6768, intron 17 of 18) |
| chr11: 130209385 | ST14 | Intron (ENST00000278742.6/6768, intron 17 of 18) |
| chr11: 130209392 | ST14 | Intron (ENST00000278742.6/6768, intron 17 of 18) |
| chr12: 1796632 | CACNA2D4 | Promoter (<=1kb) |
| chr12: 1796634 | CACNA2D4 | Promoter (<=1kb) |
| chr12: 1796641 | CACNA2D4 | Promoter (<=1kb) |
| chr12: 1796644 | CACNA2D4 | Promoter (<=1kb) |
| chr12: 1796646 | CACNA2D4 | Promoter (<=1kb) |
| chr12: 1796674 | CACNA2D4 | Promoter (<=1kb) |
| chr12: 3244606 | TSPAN9 | Intron (ENST00000649909.1/10867, intron 2 of 6) |
| chr12: 3244622 | TSPAN9 | Intron (ENST00000649909.1/10867, intron 2 of 6) |
| chr12: 3244626 | TSPAN9 | Intron (ENST00000649909.1/10867, intron 2 of 6) |
| chr12: 7291172 | ACSM4 | Distal Intergenic |
| chr12: 7291173 | ACSM4 | Distal Intergenic |
| chr12: 7440396 | CD163L1 | Intron (ENST00000313599.7/283316, intron 2 of 19) |
| chr12: 7440398 | CD163L1 | Intron (ENST00000313599.7/283316, intron 2 of 19) |
| chr12: 7440401 | CD163L1 | Intron (ENST00000313599.7/283316, intron 2 of 19) |
| chr12: 7440403 | CD163L1 | Intron (ENST00000313599.7/283316, intron 2 of 19) |
| chr12: 21941433 | ABCC9 | Promoter (<=1kb) |
| chr12: 21941437 | ABCC9 | Promoter (<=1kb) |
| chr12: 21941620 | ABCC9 | Promoter (<=1kb) |
| chr12: 21941630 | ABCC9 | Promoter (<=1kb) |
| chr12: 21941645 | ABCC9 | Promoter (<=1kb) |
| chr12: 21942141 | ABCC9 | Promoter (<=1kb) |
| chr12: 21942148 | ABCC9 | Promoter (<=1kb) |
| chr12: 21942161 | ABCC9 | Promoter (<=1kb) |
| chr12: 21942178 | ABCC9 | Promoter (<=1kb) |
| chr12: 43551605 | ADAMTS20 | Promoter (<=1kb) |
| chr12: 43551611 | ADAMTS20 | Promoter (<=1kb) |
| chr12: 43551614 | ADAMTS20 | Promoter (<=1kb) |
| chr12: 43551633 | ADAMTS20 | Promoter (<=1kb) |
| chr12: 43551638 | ADAMTS20 | Promoter (<=1kb) |
| chr12: 43551647 | ADAMTS20 | Promoter (<=1kb) |
| chr12: 43551649 | ADAMTS20 | Promoter (<=1kb) |
| chr12: 43551651 | ADAMTS20 | Promoter (<=1kb) |
| chr12: 43551653 | ADAMTS20 | Promoter (<=1kb) |
| chr12: 51721659 | SCN8A | Exon (ENST00000667214.1/6334, exon 12 of 12) |
| chr12: 51721661 | SCN8A | Exon (ENST00000667214.1/6334, exon 12 of 12) |
| chr12: 51721668 | SCN8A | Exon (ENST00000667214.1/6334, exon 12 of 12) |
| chr12: 51721679 | SCN8A | Exon (ENST00000667214.1/6334, exon 12 of 12) |
| chr12: 53938984 | HOXC13 | Promoter (<=1kb) |
| chr12: 53938990 | HOXC13 | Promoter (<=1kb) |
| chr12: 53939015 | HOXC13 | Promoter (<=1kb) |
| chr12: 53939117 | HOXC13 | Promoter (<=1kb) |
| chr12: 53973676 | HOXC11 | Promoter (<=1kb) |
| chr12: 53973713 | HOXC11 | Promoter (<=1kb) |
| chr12: 53973722 | HOXC11 | Promoter (<=1kb) |
| chr12: 53974254 | HOTAIR | Promoter (<=1kb) |
| chr12: 54000568 | HOXC9 | Promoter (<=1kb) |
| chr12: 54000569 | HOXC9 | Promoter (<=1kb) |
| chr12: 54000570 | HOXC9 | Promoter (<=1kb) |
| chr12: 54000571 | HOXC9 | Promoter (<=1kb) |
| chr12: 54000572 | HOXC9 | Promoter (<=1kb) |
| chr12: 54000578 | HOXC9 | Promoter (<=1kb) |
| chr12: 54000579 | HOXC9 | Promoter (<=1kb) |
| chr12: 54000644 | HOXC9 | Promoter (<=1kb) |
| chr12: 54000645 | HOXC9 | Promoter (<=1kb) |
| chr12: 54000649 | HOXC9 | Promoter (<=1kb) |
| chr12: 54000650 | HOXC9 | Promoter (<=1kb) |
| chr12: 57627715 | B4GALNT1 | Promoter (1-2kb) |
| chr12: 57627733 | B4GALNT1 | Promoter (<=1kb) |
| chr12: 57627751 | B4GALNT1 | Promoter (<=1kb) |
| chr12: 57627753 | B4GALNT1 | Promoter (<=1kb) |
| chr12: 57627840 | B4GALNT1 | Promoter (<=1kb) |
| chr12: 57627842 | B4GALNT1 | Promoter (<=1kb) |
| chr12: 57627847 | B4GALNT1 | Promoter (<=1kb) |
| chr12: 57627849 | B4GALNT1 | Promoter (<=1kb) |
| chr12: 57865324 | LOC283387 | Distal Intergenic |
| chr12: 57865340 | LOC283387 | Distal Intergenic |
| chr12: 57865341 | LOC283387 | Distal Intergenic |
| chr12: 57865349 | LOC283387 | Distal Intergenic |
| chr12: 57865350 | LOC283387 | Distal Intergenic |
| chr12: 57865356 | LOC283387 | Distal Intergenic |
| chr12: 57865357 | LOC283387 | Distal Intergenic |
| chr12: 57865460 | LOC283387 | Distal Intergenic |
| chr12: 57865461 | LOC283387 | Distal Intergenic |
| chr12: 57865466 | LOC283387 | Distal Intergenic |
| chr12: 57865467 | LOC283387 | Distal Intergenic |
| chr12: 57865472 | LOC283387 | Distal Intergenic |
| chr12: 57865473 | LOC283387 | Distal Intergenic |
| chr12: 57865484 | LOC283387 | Distal Intergenic |
| chr12: 57865485 | LOC283387 | Distal Intergenic |
| chr12: 57865486 | LOC283387 | Distal Intergenic |
| chr12: 57865487 | LOC283387 | Distal Intergenic |
| chr12: 57865491 | LOC283387 | Distal Intergenic |
| chr12: 57865492 | LOC283387 | Distal Intergenic |
| chr12: 57865493 | LOC283387 | Distal Intergenic |
| chr12: 57865494 | LOC283387 | Distal Intergenic |
| chr12: 57865498 | LOC283387 | Distal Intergenic |
| chr12: 57865499 | LOC283387 | Distal Intergenic |
| chr12: 57865541 | LOC283387 | Distal Intergenic |
| chr12: 57865543 | LOC283387 | Distal Intergenic |
| chr12: 57865547 | LOC283387 | Distal Intergenic |
| chr12: 57865569 | LOC283387 | Distal Intergenic |
| chr12: 57865575 | LOC283387 | Distal Intergenic |
| chr12: 57865588 | LOC283387 | Distal Intergenic |
| chr12: 57865589 | LOC283387 | Distal Intergenic |
| chr12: 62631991 | LINC01465 | Distal Intergenic |
| chr12: 62632007 | LINC01465 | Distal Intergenic |
| chr12: 62632008 | LINC01465 | Distal Intergenic |
| chr12: 62632010 | LINC01465 | Distal Intergenic |
| chr12: 62632011 | LINC01465 | Distal Intergenic |
| chr12: 110688833 | HVCN1 | Promoter (<=1kb) |
| chr12: 110688840 | HVCN1 | Promoter (<=1kb) |
| chr12: 110688842 | HVCN1 | Promoter (<=1kb) |
| chr12: 110688873 | HVCN1 | Promoter (<=1kb) |
| chr12: 110688890 | HVCN1 | Promoter (<=1kb) |
| chr12: 110688894 | HVCN1 | Promoter (<=1kb) |
| chr12: 110688896 | HVCN1 | Promoter (<=1kb) |
| chr12: 110688905 | HVCN1 | Promoter (<=1kb) |
| chr12: 110688914 | HVCN1 | Promoter (<=1kb) |
| chr12: 110688924 | HVCN1 | Promoter (<=1kb) |
| chr12: 110689040 | HVCN1 | Promoter (<=1kb) |
| chr12: 110689047 | HVCN1 | Promoter (<=1kb) |
| chr12: 110689054 | HVCN1 | Promoter (<=1kb) |
| chr12: 110689061 | HVCN1 | Promoter (<=1kb) |
| chr12: 110689084 | HVCN1 | Promoter (<=1kb) |
| chr12: 110689093 | HVCN1 | Promoter (<=1kb) |
| chr12: 110689094 | HVCN1 | Promoter (<=1kb) |
| chr12: 110689097 | HVCN1 | Promoter (<=1kb) |
| chr12: 110689098 | HVCN1 | Promoter (<=1kb) |
| chr12: 110689099 | HVCN1 | Promoter (<=1kb) |
| chr12: 110689100 | HVCN1 | Promoter (<=1kb) |
| chr12: 110689106 | HVCN1 | Promoter (<=1kb) |
| chr12: 110689107 | HVCN1 | Promoter (<=1kb) |
| chr12: 110689111 | HVCN1 | Promoter (<=1kb) |
| chr12: 110689112 | HVCN1 | Promoter (<=1kb) |
| chr12: 110689187 | HVCN1 | Promoter (<=1kb) |
| chr12: 110689193 | HVCN1 | Promoter (<=1kb) |
| chr12: 110689199 | HVCN1 | Promoter (<=1kb) |
| chr12: 110689205 | HVCN1 | Promoter (<=1kb) |
| chr12: 110689218 | HVCN1 | Promoter (<=1kb) |
| chr12: 116698253 | C12orf49 | Distal Intergenic |
| chr12: 116698254 | C12orf49 | Distal Intergenic |
| chr12: 116698256 | C12orf49 | Distal Intergenic |
| chr12: 116698257 | C12orf49 | Distal Intergenic |
| chr12: 116698277 | C12orf49 | Distal Intergenic |
| chr12: 116698278 | C12orf49 | Distal Intergenic |
| chr12: 118131915 | VSIG10 | Intron (ENST00000542011.1/54621, intron 2 of 3) |
| chr12: 118131927 | VSIG10 | Intron (ENST00000542011.1/54621, intron 2 of 3) |
| chr12: 121227419 | P2RX4 | Intron (ENST00000538701.5/5025, intron 1 of 3) |
| chr12: 121227431 | P2RX4 | Intron (ENST00000538701.5/5025, intron 1 of 3) |
| chr12: 130163471 | FZD10 | Promoter (1-2kb) |
| chr12: 130163473 | FZD10 | Promoter (1-2kb) |
| chr12: 132904798 | CHFR | Promoter (<=1kb) |
| chr12: 132904799 | CHFR | Promoter (<=1kb) |
| chr12: 132904804 | CHFR | Promoter (<=1kb) |
| chr12: 132904805 | CHFR | Promoter (<=1kb) |
| chr12: 132904807 | CHFR | Promoter (<=1kb) |
| chr13: 78595986 | LOC780529 | Promoter (<=1kb) |
| chr13: 78596001 | LOC780529 | Promoter (<=1kb) |
| chr13: 78596003 | LOC780529 | Promoter (<=1kb) |
| chr13: 78596011 | LOC780529 | Promoter (<=1kb) |
| chr13: 78596024 | LOC780529 | Promoter (<=1kb) |
| chr13: 78596050 | LOC780529 | Promoter (<=1kb) |
| chr13: 78596058 | LOC780529 | Promoter (<=1kb) |
| chr13: 78596069 | LOC780529 | Promoter (<=1kb) |
| chr13: 78596078 | LOC780529 | Promoter (<=1kb) |
| chr13: 78596080 | LOC780529 | Promoter (<=1kb) |
| chr13: 78596104 | LOC780529 | Promoter (<=1kb) |
| chr13: 78596109 | LOC780529 | Promoter (<=1kb) |
| chr13: 78596115 | LOC780529 | Promoter (<=1kb) |
| chr13: 78596129 | LOC780529 | Promoter (<=1kb) |
| chr13: 78596133 | LOC780529 | Promoter (<=1kb) |
| chr13: 78596135 | LOC780529 | Promoter (<=1kb) |
| chr13: 78596137 | LOC780529 | Promoter (<=1kb) |
| chr13: 78596232 | LOC780529 | Promoter (<=1kb) |
| chr13: 78596236 | LOC780529 | Promoter (<=1kb) |
| chr13: 78596244 | LOC780529 | Promoter (<=1kb) |
| chr13: 78596250 | LOC780529 | Promoter (<=1kb) |
| chr13: 109400373 | LINC00399 | Promoter (<=1kb) |
| chr13: 109400374 | LINC00399 | Promoter (<=1kb) |
| chr13: 109400375 | LINC00399 | Promoter (<=1kb) |
| chr13: 109400376 | LINC00399 | Promoter (<=1kb) |
| chr13: 112104494 | SOX1-OT | Promoter (2-3kb) |
| chr13: 112104508 | SOX1-OT | Promoter (2-3kb) |
| chr13: 112104673 | SOX1-OT | Promoter (2-3kb) |
| chr13: 112104683 | SOX1-OT | Promoter (2-3kb) |
| chr13: 112104685 | SOX1-OT | Promoter (2-3kb) |
| chr13: 112104691 | SOX1-OT | Promoter (2-3kb) |
| chr13: 112104700 | SOX1-OT | Promoter (2-3kb) |
| chr13: 112104721 | SOX1-OT | Promoter (2-3kb) |
| chr13: 112104725 | SOX1-OT | Promoter (2-3kb) |
| chr13: 112104727 | SOX1-OT | Promoter (2-3kb) |
| chr13: 112104782 | SOX1-OT | Promoter (2-3kb) |
| chr13: 112104788 | SOX1-OT | Promoter (2-3kb) |
| chr13: 112104794 | SOX1-OT | Promoter (2-3kb) |
| chr13: 112104799 | SOX1-OT | Promoter (2-3kb) |
| chr13: 112104811 | SOX1-OT | Promoter (2-3kb) |
| chr13: 112888853 | MCF2L | Distal Intergenic |
| chr13: 112888855 | MCF2L | Distal Intergenic |
| chr13: 112888866 | MCF2L | Distal Intergenic |
| chr13: 112888875 | MCF2L | Distal Intergenic |
| chr13: 113549498 | TMCO3 | Promoter (1-2kb) |
| chr13: 113549524 | TMCO3 | Promoter (1-2kb) |
| chr13: 113549544 | TMCO3 | Promoter (1-2kb) |
| chr13: 113549558 | TMCO3 | Promoter (1-2kb) |
| chr14: 23576274 | JPH4 | Promoter (1-2kb) |
| chr14: 23576292 | JPH4 | Promoter (1-2kb) |
| chr14: 23576360 | JPH4 | Promoter (1-2kb) |
| chr14: 23576365 | JPH4 | Promoter (1-2kb) |
| chr14: 23576369 | JPH4 | Promoter (1-2kb) |
| chr14: 23576380 | JPH4 | Promoter (1-2kb) |
| chr14: 23576406 | JPH4 | Promoter (1-2kb) |
| chr14: 36508416 | SFTA3 | Intron (ENST00000634305.1/ENST00000634305.1, intron 3 of 3) |
| chr14: 36508418 | SFTA3 | Intron (ENST00000634305.1/ENST00000634305.1, intron 3 of 3) |
| chr14: 36508424 | SFTA3 | Intron (ENST00000634305.1/ENST00000634305.1, intron 3 of 3) |
| chr14: 36508426 | SFTA3 | Intron (ENST00000634305.1/ENST00000634305.1, intron 3 of 3) |
| chr14: 36508431 | SFTA3 | Intron (ENST00000634305.1/ENST00000634305.1, intron 3 of 3) |
| chr14: 36508435 | SFTA3 | Intron (ENST00000634305.1/ENST00000634305.1, intron 3 of 3) |
| chr14: 36508442 | SFTA3 | Intron (ENST00000634305.1/ENST00000634305.1, intron 3 of 3) |
| chr14: 36508448 | SFTA3 | Intron (ENST00000634305.1/ENST00000634305.1, intron 3 of 3) |
| chr14: 37588224 | FOXA1 | Downstream (1-2kb) |
| chr14: 37588225 | FOXA1 | Downstream (1-2kb) |
| chr14: 37588226 | FOXA1 | Downstream (1-2kb) |
| chr14: 37588227 | FOXA1 | Downstream (1-2kb) |
| chr14: 37588233 | FOXA1 | Downstream (1-2kb) |
| chr14: 37588234 | FOXA1 | Downstream (1-2kb) |
| chr14: 37588235 | FOXA1 | Downstream (1-2kb) |
| chr14: 37588240 | FOXA1 | Downstream (1-2kb) |
| chr14: 37588248 | FOXA1 | Downstream (1-2kb) |
| chr14: 64038597 | SYNE2 | Intron (ENST00000358025.7/23224, intron 45 of 115) |
| chr14: 64038605 | SYNE2 | Intron (ENST00000358025.7/23224, intron 45 of 115) |
| chr14: 64038606 | SYNE2 | Intron (ENST00000358025.7/23224, intron 45 of 115) |
| chr14: 64038623 | SYNE2 | Intron (ENST00000358025.7/23224, intron 45 of 115) |
| chr14: 64038624 | SYNE2 | Intron (ENST00000358025.7/23224, intron 45 of 115) |
| chr14: 64038626 | SYNE2 | Intron (ENST00000358025.7/23224, intron 45 of 115) |
| chr14: 64038650 | SYNE2 | Intron (ENST00000358025.7/23224, intron 45 of 115) |
| chr14: 76961840 | LINC01629 | Promoter (2-3kb) |
| chr14: 76961853 | LINC01629 | Promoter (2-3kb) |
| chr14: 76961867 | LINC01629 | Promoter (2-3kb) |
| chr14: 76961872 | LINC01629 | Promoter (2-3kb) |
| chr14: 76961883 | LINC01629 | Promoter (2-3kb) |
| chr14: 76961884 | LINC01629 | Promoter (2-3kb) |
| chr14: 76961885 | LINC01629 | Promoter (2-3kb) |
| chr14: 76961886 | LINC01629 | Promoter (2-3kb) |
| chr14: 76961993 | LINC01629 | Promoter (2-3kb) |
| chr14: 76961999 | LINC01629 | Promoter (2-3kb) |
| chr14: 76962008 | LINC01629 | Promoter (2-3kb) |
| chr14: 76962016 | LINC01629 | Promoter (2-3kb) |
| chr14: 76962031 | LINC01629 | Promoter (2-3kb) |
| chr14: 77125128 | TMEM63C | Intron (ENST00000557752.1/57156, intron 2 of 5) |
| chr14: 77125135 | TMEM63C | Intron (ENST00000557752.1/57156, intron 2 of 5) |
| chr14: 77125139 | TMEM63C | Intron (ENST00000557752.1/57156, intron 2 of 5) |
| chr14: 77125141 | TMEM63C | Intron (ENST00000557752.1/57156, intron 2 of 5) |
| chr14: 77125151 | TMEM63C | Intron (ENST00000557752.1/57156, intron 2 of 5) |
| chr14: 77125210 | TMEM63C | Intron (ENST00000557752.1/57156, intron 2 of 5) |
| chr14: 77125215 | TMEM63C | Intron (ENST00000557752.1/57156, intron 2 of 5) |
| chr14: 77125217 | TMEM63C | Intron (ENST00000557752.1/57156, intron 2 of 5) |
| chr14: 77125231 | TMEM63C | Intron (ENST00000557752.1/57156, intron 2 of 5) |
| chr14: 77125283 | TMEM63C | Intron (ENST00000557752.1/57156, intron 2 of 5) |
| chr14: 77125289 | TMEM63C | Intron (ENST00000557752.1/57156, intron 2 of 5) |
| chr14: 77125313 | TMEM63C | Intron (ENST00000557752.1/57156, intron 2 of 5) |
| chr14: 77125354 | TMEM63C | Intron (ENST00000557752.1/57156, intron 2 of 5) |
| chr14: 77125355 | TMEM63C | Intron (ENST00000557752.1/57156, intron 2 of 5) |
| chr14: 77125361 | TMEM63C | Intron (ENST00000557752.1/57156, intron 2 of 5) |
| chr14: 77125386 | TMEM63C | Intron (ENST00000557752.1/57156, intron 2 of 5) |
| chr14: 77125392 | TMEM63C | Intron (ENST00000557752.1/57156, intron 2 of 5) |
| chr14: 77125394 | TMEM63C | Intron (ENST00000557752.1/57156, intron 2 of 5) |
| chr14: 77125395 | TMEM63C | Intron (ENST00000557752.1/57156, intron 2 of 5) |
| chr14: 77125401 | TMEM63C | Intron (ENST00000557752.1/57156, intron 2 of 5) |
| chr14: 77125402 | TMEM63C | Intron (ENST00000557752.1/57156, intron 2 of 5) |
| chr14: 90600763 | TTC7B | Intron (ENST00000328459.11/145567, intron 17 of 19) |
| chr14: 90600771 | TTC7B | Intron (ENST00000328459.11/145567, intron 17 of 19) |
| chr14: 90600800 | TTC7B | Intron (ENST00000328459.11/145567, intron 17 of 19) |
| chr14: 90600805 | TTC7B | Intron (ENST00000328459.11/145567, intron 17 of 19) |
| chr14: 90600821 | TTC7B | Intron (ENST00000328459.11/145567, intron 17 of 19) |
| chr14: 90828164 | TTC7B | Exon (ENST00000659138.1/ENST00000659138.1, exon 1 of 4) |
| chr14: 90828166 | TTC7B | Exon (ENST00000659138.1/ENST00000659138.1, exon 1 of 4) |
| chr14: 90828180 | TTC7B | Exon (ENST00000659138.1/ENST00000659138.1, exon 1 of 4) |
| chr14: 90828184 | TTC7B | Exon (ENST00000659138.1/ENST00000659138.1, exon 1 of 4) |
| chr14: 90828189 | TTC7B | Exon (ENST00000659138.1/ENST00000659138.1, exon 1 of 4) |
| chr14: 90828194 | TTC7B | Exon (ENST00000659138.1/ENST00000659138.1, exon 1 of 4) |
| chr14: 90828202 | TTC7B | Distal Intergenic |
| chr14: 90828204 | TTC7B | Distal Intergenic |
| chr14: 90828207 | TTC7B | Distal Intergenic |
| chr14: 95460840 | SYNE3 | Intron (ENST00000557275.5/161176, intron 3 of 16) |
| chr14: 95460842 | SYNE3 | Intron (ENST00000557275.5/161176, intron 3 of 16) |
| chr14: 95460890 | SYNE3 | Intron (ENST00000557275.5/161176, intron 3 of 16) |
| chr14: 99737735 | EML1 | Promoter (<=1kb) |
| chr14: 99737737 | EML1 | Promoter (<=1kb) |
| chr14: 99737774 | EML1 | Promoter (<=1kb) |
| chr14: 100654824 | LINC00523 | Promoter (2-3kb) |
| chr14: 100654836 | LINC00523 | Promoter (2-3kb) |
| chr14: 100654840 | LINC00523 | Promoter (2-3kb) |
| chr14: 103879905 | LINC00637 | Exon (ENST00000660176.1/ENST00000660176.1, exon 2 of 2) |
| chr14: 103879920 | LINC00637 | Exon (ENST00000660176.1/ENST00000660176.1, exon 2 of 2) |
| chr14: 103879970 | LINC00637 | Exon (ENST00000660176.1/ENST00000660176.1, exon 2 of 2) |
| chr14: 103879986 | LINC00637 | Exon (ENST00000660176.1/ENST00000660176.1, exon 2 of 2) |
| chr14: 103880033 | LINC00637 | Exon (ENST00000660176.1/ENST00000660176.1, exon 2 of 2) |
| chr14: 104367967 | LINC02691 | Distal Intergenic |
| chr14: 104367968 | LINC02691 | Distal Intergenic |
| chr14: 104367969 | LINC02691 | Distal Intergenic |
| chr14: 104367970 | LINC02691 | Distal Intergenic |
| chr14: 104367972 | LINC02691 | Distal Intergenic |
| chr14: 104367973 | LINC02691 | Distal Intergenic |
| chr14: 104367994 | LINC02691 | Distal Intergenic |
| chr14: 104367995 | LINC02691 | Distal Intergenic |
| chr14: 104367996 | LINC02691 | Distal Intergenic |
| chr14: 104367997 | LINC02691 | Distal Intergenic |
| chr14: 104368028 | LINC02691 | Distal Intergenic |
| chr14: 104368033 | LINC02691 | Distal Intergenic |
| chr14: 104368089 | LINC02691 | Distal Intergenic |
| chr14: 104368099 | LINC02691 | Distal Intergenic |
| chr14: 105196737 | NUDT14 | Distal Intergenic |
| chr14: 105196741 | NUDT14 | Distal Intergenic |
| chr14: 105196743 | NUDT14 | Distal Intergenic |
| chr14: 105196756 | NUDT14 | Distal Intergenic |
| chr14: 105196757 | NUDT14 | Distal Intergenic |
| chr14: 105196767 | NUDT14 | Distal Intergenic |
| chr14: 105196768 | NUDT14 | Distal Intergenic |
| chr14: 105196769 | NUDT14 | Distal Intergenic |
| chr14: 105196770 | NUDT14 | Distal Intergenic |
| chr14: 105196788 | NUDT14 | Distal Intergenic |
| chr14: 105196790 | NUDT14 | Distal Intergenic |
| chr14: 105196793 | NUDT14 | Distal Intergenic |
| chr14: 105196903 | NUDT14 | Distal Intergenic |
| chr14: 105196917 | NUDT14 | Distal Intergenic |
| chr14: 105196918 | NUDT14 | Distal Intergenic |
| chr14: 105196923 | NUDT14 | Distal Intergenic |
| chr14: 105196924 | NUDT14 | Distal Intergenic |
| chr14: 105196928 | NUDT14 | Distal Intergenic |
| chr14: 105196929 | NUDT14 | Distal Intergenic |
| chr14: 105196953 | NUDT14 | Distal Intergenic |
| chr15: 67835522 | SKOR1 | Promoter (2-3kb) |
| chr15: 67835527 | SKOR1 | Promoter (2-3kb) |
| chr15: 67835540 | SKOR1 | Promoter (2-3kb) |
| chr15: 67835550 | SKOR1 | Promoter (2-3kb) |
| chr15: 67835558 | SKOR1 | Promoter (2-3kb) |
| chr15: 67835608 | SKOR1 | Promoter (2-3kb) |
| chr15: 76347141 | SCAPER | Downstream (<1kb) |
| chr15: 76347145 | SCAPER | Downstream (<1kb) |
| chr15: 76347154 | SCAPER | Downstream (<1kb) |
| chr15: 76347156 | SCAPER | Downstream (<1kb) |
| chr15: 76347166 | SCAPER | Downstream (<1kb) |
| chr15: 76347263 | SCAPER | Downstream (<1kb) |
| chr15: 76347265 | SCAPER | Downstream (<1kb) |
| chr15: 76347272 | SCAPER | Downstream (<1kb) |
| chr15: 76347291 | SCAPER | Downstream (<1kb) |
| chr15: 89407577 | MIR9-3HG | Distal Intergenic |
| chr15: 89407579 | MIR9-3HG | Distal Intergenic |
| chr15: 89407583 | MIR9-3HG | Distal Intergenic |
| chr15: 89408147 | MIR9-3HG | Distal Intergenic |
| chr15: 89408149 | MIR9-3HG | Distal Intergenic |
| chr15: 89408158 | MIR9-3HG | Distal Intergenic |
| chr15: 89408160 | MIR9-3HG | Distal Intergenic |
| chr15: 89408170 | MIR9-3HG | Distal Intergenic |
| chr15: 89408173 | MIR9-3HG | Distal Intergenic |
| chr15: 89408185 | MIR9-3HG | Distal Intergenic |
| chr16: 64422 | RHBDF1 | Promoter (<=1kb) |
| chr16: 64426 | RHBDF1 | Promoter (<=1kb) |
| chr16: 1679822 | JPT2 | Promoter (<=1kb) |
| chr16: 1679838 | JPT2 | Promoter (<=1kb) |
| chr16: 2224654 | E4F1 | Promoter (<=1kb) |
| chr16: 2224662 | E4F1 | Promoter (<=1kb) |
| chr16: 2224666 | E4F1 | Promoter (<=1kb) |
| chr16: 2224689 | E4F1 | Promoter (<=1kb) |
| chr16: 2603395 | LOC652276 | Promoter (<=1kb) |
| chr16: 2603404 | LOC652276 | Promoter (<=1kb) |
| chr16: 2603405 | LOC652276 | Promoter (<=1kb) |
| chr16: 2603414 | LOC652276 | Promoter (<=1kb) |
| chr16: 2603444 | LOC652276 | Promoter (<=1kb) |
| chr16: 2603476 | LOC652276 | Promoter (<=1kb) |
| chr16: 2603622 | LOC652276 | Promoter (<=1kb) |
| chr16: 2603625 | LOC652276 | Promoter (<=1kb) |
| chr16: 2603654 | LOC652276 | Promoter (<=1kb) |
| chr16: 2603657 | LOC652276 | Promoter (<=1kb) |
| chr16: 2603673 | LOC652276 | Promoter (<=1kb) |
| chr16: 2603704 | LOC652276 | Promoter (<=1kb) |
| chr16: 4893573 | PPL | Promoter (<=1kb) |
| chr16: 4893574 | PPL | Promoter (<=1kb) |
| chr16: 4893584 | PPL | Promoter (<=1kb) |
| chr16: 4893585 | PPL | Promoter (<=1kb) |
| chr16: 4893588 | PPL | Promoter (<=1kb) |
| chr16: 4893589 | PPL | Promoter (<=1kb) |
| chr16: 4893613 | PPL | Promoter (<=1kb) |
| chr16: 4893617 | PPL | Promoter (<=1kb) |
| chr16: 25066728 | LINC02175 | Promoter (<=1kb) |
| chr16: 25066736 | LINC02175 | Promoter (<=1kb) |
| chr16: 25066739 | LINC02175 | Promoter (<=1kb) |
| chr16: 25066742 | LINC02175 | Promoter (<=1kb) |
| chr16: 30005475 | DOC2A | Promoter (1-2kb) |
| chr16: 30005481 | DOC2A | Promoter (1-2kb) |
| chr16: 30429550 | DCTPP1 | Promoter (<=1kb) |
| chr16: 30429572 | DCTPP1 | Promoter (<=1kb) |
| chr16: 30429585 | DCTPP1 | Promoter (<=1kb) |
| chr16: 30897175 | CTF1 | Promoter (<=1kb) |
| chr16: 30897178 | CTF1 | Promoter (<=1kb) |
| chr16: 30897179 | CTF1 | Promoter (<=1kb) |
| chr16: 30897180 | CTF1 | Promoter (<=1kb) |
| chr16: 30897181 | CTF1 | Promoter (<=1kb) |
| chr16: 30897182 | CTF1 | Promoter (<=1kb) |
| chr16: 30897183 | CTF1 | Promoter (<=1kb) |
| chr16: 30897189 | CTF1 | Promoter (<=1kb) |
| chr16: 30897190 | CTF1 | Promoter (<=1kb) |
| chr16: 51134634 | SALL1 | Downstream (1-2kb) |
| chr16: 51134643 | SALL1 | Downstream (1-2kb) |
| chr16: 51134645 | SALL1 | Downstream (1-2kb) |
| chr16: 51134732 | SALL1 | Downstream (1-2kb) |
| chr16: 51134734 | SALL1 | Downstream (1-2kb) |
| chr16: 69965499 | CLEC18A | Distal Intergenic |
| chr16: 69965517 | CLEC18A | Distal Intergenic |
| chr16: 69965522 | CLEC18A | Distal Intergenic |
| chr16: 69965526 | CLEC18A | Distal Intergenic |
| chr16: 69965531 | CLEC18A | Distal Intergenic |
| chr16: 73063101 | ZFHX3 | Intron (ENST00000641206.2/463, intron 8 of 17) |
| chr16: 73063102 | ZFHX3 | Intron (ENST00000641206.2/463, intron 8 of 17) |
| chr16: 73063116 | ZFHX3 | Intron (ENST00000641206.2/463, intron 8 of 17) |
| chr16: 73063117 | ZFHX3 | Intron (ENST00000641206.2/463, intron 8 of 17) |
| chr16: 73063119 | ZFHX3 | Intron (ENST00000641206.2/463, intron 8 of 17) |
| chr16: 73063121 | ZFHX3 | Intron (ENST00000641206.2/463, intron 8 of 17) |
| chr16: 73063139 | ZFHX3 | Intron (ENST00000641206.2/463, intron 8 of 17) |
| chr16: 74407173 | CLEC18B | Downstream (1-2kb) |
| chr16: 74407175 | CLEC18B | Downstream (1-2kb) |
| chr16: 74407181 | CLEC18B | Downstream (1-2kb) |
| chr16: 74407183 | CLEC18B | Downstream (1-2kb) |
| chr16: 74407190 | CLEC18B | Downstream (1-2kb) |
| chr16: 81431801 | CMIP | Distal Intergenic |
| chr16: 81431802 | CMIP | Distal Intergenic |
| chr16: 81431809 | CMIP | Distal Intergenic |
| chr16: 81431810 | CMIP | Distal Intergenic |
| chr16: 81431822 | CMIP | Distal Intergenic |
| chr16: 85690021 | GINS2 | Promoter (<=1kb) |
| chr16: 85690041 | GINS2 | Promoter (<=1kb) |
| chr16: 86578985 | FOXL1 | Promoter (<=1kb) |
| chr16: 86579015 | FOXL1 | Promoter (<=1kb) |
| chr16: 86579035 | FOXL1 | Promoter (<=1kb) |
| chr16: 86579395 | FOXL1 | Promoter (<=1kb) |
| chr16: 86579396 | FOXL1 | Promoter (<=1kb) |
| chr16: 86579410 | FOXL1 | Promoter (<=1kb) |
| chr16: 87706098 | KLHDC4 | Promoter (2-3kb) |
| chr16: 87706102 | KLHDC4 | Promoter (2-3kb) |
| chr16: 89657050 | CHMP1A | Promoter (<=1kb) |
| chr16: 89657057 | CHMP1A | Promoter (<=1kb) |
| chr16: 89657064 | CHMP1A | Promoter (<=1kb) |
| chr16: 89657120 | CHMP1A | Promoter (<=1kb) |
| chr17: 17836673 | SREBF1 | Promoter (<=1kb) |
| chr17: 17836684 | SREBF1 | Promoter (<=1kb) |
| chr17: 17836686 | SREBF1 | Promoter (<=1kb) |
| chr17: 17836695 | SREBF1 | Promoter (<=1kb) |
| chr17: 17836730 | SREBF1 | Promoter (<=1kb) |
| chr17: 17836797 | SREBF1 | Promoter (<=1kb) |
| chr17: 17836812 | SREBF1 | Promoter (<=1kb) |
| chr17: 44776152 | ADAM11 | Promoter (<=1kb) |
| chr17: 44776160 | ADAM11 | Promoter (<=1kb) |
| chr17: 44776166 | ADAM11 | Promoter (<=1kb) |
| chr17: 44776172 | ADAM11 | Promoter (<=1kb) |
| chr17: 44776178 | ADAM11 | Promoter (<=1kb) |
| chr17: 44776180 | ADAM11 | Promoter (<=1kb) |
| chr17: 44776221 | ADAM11 | Promoter (<=1kb) |
| chr17: 44776224 | ADAM11 | Promoter (<=1kb) |
| chr17: 44776243 | ADAM11 | Promoter (<=1kb) |
| chr17: 44776244 | ADAM11 | Promoter (<=1kb) |
| chr17: 44776264 | ADAM11 | Promoter (<=1kb) |
| chr17: 44776265 | ADAM11 | Promoter (<=1kb) |
| chr17: 45133276 | PLCD3 | Promoter (<=1kb) |
| chr17: 45133280 | PLCD3 | Promoter (<=1kb) |
| chr17: 45133282 | PLCD3 | Promoter (<=1kb) |
| chr17: 45133294 | PLCD3 | Promoter (<=1kb) |
| chr17: 45133308 | PLCD3 | Promoter (<=1kb) |
| chr17: 45133316 | PLCD3 | Promoter (<=1kb) |
| chr17: 48550618 | HOXB-AS3 | Promoter (<=1kb) |
| chr17: 48550634 | HOXB-AS3 | Promoter (1-2kb) |
| chr17: 48550638 | HOXB-AS3 | Promoter (1-2kb) |
| chr17: 48550649 | HOXB-AS3 | Promoter (1-2kb) |
| chr17: 48550653 | HOXB-AS3 | Promoter (1-2kb) |
| chr17: 48550661 | HOXB-AS3 | Promoter (1-2kb) |
| chr17: 48550676 | HOXB-AS3 | Promoter (1-2kb) |
| chr17: 48550681 | HOXB-AS3 | Promoter (1-2kb) |
| chr17: 48550684 | HOXB-AS3 | Promoter (1-2kb) |
| chr17: 48550869 | HOXB-AS3 | Promoter (1-2kb) |
| chr17: 48550912 | HOXB-AS3 | Promoter (1-2kb) |
| chr17: 48550941 | HOXB-AS3 | Promoter (1-2kb) |
| chr17: 48550942 | HOXB-AS3 | Promoter (1-2kb) |
| chr17: 48550945 | HOXB-AS3 | Promoter (1-2kb) |
| chr17: 48550946 | HOXB-AS3 | Promoter (1-2kb) |
| chr17: 48550978 | HOXB-AS3 | Promoter (1-2kb) |
| chr17: 48550981 | HOXB-AS3 | Promoter (1-2kb) |
| chr17: 48551037 | HOXB-AS3 | Promoter (1-2kb) |
| chr17: 48551039 | HOXB-AS3 | Promoter (1-2kb) |
| chr17: 48551043 | HOXB-AS3 | Promoter (1-2kb) |
| chr17: 48551049 | HOXB-AS3 | Promoter (1-2kb) |
| chr17: 48551052 | HOXB-AS3 | Promoter (1-2kb) |
| chr17: 48551056 | HOXB-AS3 | Promoter (1-2kb) |
| chr17: 48551058 | HOXB-AS3 | Promoter (1-2kb) |
| chr17: 48551062 | HOXB-AS3 | Promoter (1-2kb) |
| chr17: 48551070 | HOXB-AS3 | Promoter (1-2kb) |
| chr17: 48578036 | HOXB4 | Promoter (<=1kb) |
| chr17: 48578046 | HOXB4 | Promoter (<=1kb) |
| chr17: 48578069 | HOXB4 | Promoter (<=1kb) |
| chr17: 48578075 | HOXB4 | Promoter (<=1kb) |
| chr17: 48578078 | HOXB4 | Promoter (<=1kb) |
| chr17: 48578090 | HOXB4 | Promoter (<=1kb) |
| chr17: 48578096 | HOXB4 | Promoter (<=1kb) |
| chr17: 48578099 | HOXB4 | Promoter (<=1kb) |
| chr17: 48578123 | HOXB4 | Promoter (<=1kb) |
| chr17: 48578125 | HOXB4 | Promoter (<=1kb) |
| chr17: 48578130 | HOXB4 | Promoter (<=1kb) |
| chr17: 48578137 | HOXB4 | Promoter (<=1kb) |
| chr17: 48578144 | HOXB4 | Promoter (<=1kb) |
| chr17: 48578147 | HOXB4 | Promoter (<=1kb) |
| chr17: 48578149 | HOXB4 | Promoter (<=1kb) |
| chr17: 48578150 | HOXB4 | Promoter (<=1kb) |
| chr17: 48578151 | HOXB4 | Promoter (<=1kb) |
| chr17: 48578154 | HOXB4 | Promoter (<=1kb) |
| chr17: 48578158 | HOXB4 | Promoter (<=1kb) |
| chr17: 48578165 | HOXB4 | Promoter (<=1kb) |
| chr17: 48578462 | HOXB4 | Promoter (<=1kb) |
| chr17: 48578468 | HOXB4 | Promoter (<=1kb) |
| chr17: 48578638 | HOXB4 | Promoter (<=1kb) |
| chr17: 48578650 | HOXB4 | Promoter (<=1kb) |
| chr17: 48578652 | HOXB4 | Promoter (<=1kb) |
| chr17: 48578654 | HOXB4 | Promoter (<=1kb) |
| chr17: 48578663 | HOXB4 | Promoter (<=1kb) |
| chr17: 48578685 | HOXB4 | Promoter (<=1kb) |
| chr17: 48578697 | HOXB4 | Promoter (<=1kb) |
| chr17: 48581942 | MIR10A | Promoter (<=1kb) |
| chr17: 48581956 | MIR10A | Promoter (<=1kb) |
| chr17: 48581999 | MIR10A | Promoter (<=1kb) |
| chr17: 48582001 | MIR10A | Promoter (<=1kb) |
| chr17: 48582003 | MIR10A | Promoter (<=1kb) |
| chr17: 48582024 | MIR10A | Promoter (<=1kb) |
| chr17: 48582192 | MIR10A | Promoter (<=1kb) |
| chr17: 48582200 | MIR10A | Promoter (<=1kb) |
| chr17: 48582204 | MIR10A | Promoter (<=1kb) |
| chr17: 48582210 | MIR10A | Promoter (<=1kb) |
| chr17: 48582236 | MIR10A | Promoter (<=1kb) |
| chr17: 48582239 | MIR10A | Promoter (<=1kb) |
| chr17: 48582242 | MIR10A | Promoter (<=1kb) |
| chr17: 48582245 | MIR10A | Promoter (<=1kb) |
| chr17: 48582247 | MIR10A | Promoter (<=1kb) |
| chr17: 48582273 | MIR10A | Promoter (<=1kb) |
| chr17: 48582275 | MIR10A | Promoter (<=1kb) |
| chr17: 48582279 | MIR10A | Promoter (<=1kb) |
| chr17: 48582394 | MIR10A | Promoter (<=1kb) |
| chr17: 48582395 | MIR10A | Promoter (<=1kb) |
| chr17: 48582396 | MIR10A | Promoter (<=1kb) |
| chr17: 48582397 | MIR10A | Promoter (<=1kb) |
| chr17: 48582399 | MIR10A | Promoter (<=1kb) |
| chr17: 48582400 | MIR10A | Promoter (<=1kb) |
| chr17: 48582402 | MIR10A | Promoter (<=1kb) |
| chr17: 48582403 | MIR10A | Promoter (<=1kb) |
| chr17: 48582414 | MIR10A | Promoter (<=1kb) |
| chr17: 48582415 | MIR10A | Promoter (<=1kb) |
| chr17: 48592664 | HOXB-AS3 | Promoter (<=1kb) |
| chr17: 48592684 | HOXB-AS3 | Promoter (<=1kb) |
| chr17: 48592685 | HOXB-AS3 | Promoter (<=1kb) |
| chr17: 48592719 | HOXB-AS3 | Promoter (<=1kb) |
| chr17: 48592723 | HOXB-AS3 | Promoter (<=1kb) |
| chr17: 48592727 | HOXB-AS3 | Promoter (<=1kb) |
| chr17: 48633961 | HOXB7 | Promoter (<=1kb) |
| chr17: 48633996 | HOXB7 | Promoter (<=1kb) |
| chr17: 48633999 | HOXB7 | Promoter (<=1kb) |
| chr17: 48634007 | HOXB7 | Promoter (<=1kb) |
| chr17: 48634029 | HOXB7 | Promoter (<=1kb) |
| chr17: 48719037 | PRAC1 | Downstream (2-3kb) |
| chr17: 48719038 | PRAC1 | Downstream (2-3kb) |
| chr17: 48719043 | PRAC1 | Downstream (2-3kb) |
| chr17: 48719050 | PRAC1 | Downstream (2-3kb) |
| chr17: 48719294 | PRAC1 | Downstream (2-3kb) |
| chr17: 48719300 | PRAC1 | Downstream (2-3kb) |
| chr17: 48719354 | PRAC1 | Downstream (2-3kb) |
| chr17: 48754998 | HOXB13 | Distal Intergenic |
| chr17: 48755007 | HOXB13 | Distal Intergenic |
| chr17: 48755023 | HOXB13 | Distal Intergenic |
| chr17: 48755032 | HOXB13 | Distal Intergenic |
| chr17: 48755052 | HOXB13 | Distal Intergenic |
| chr17: 48755064 | HOXB13 | Distal Intergenic |
| chr17: 48755066 | HOXB13 | Distal Intergenic |
| chr17: 49910367 | DLX4 | Distal Intergenic |
| chr17: 49910377 | DLX4 | Distal Intergenic |
| chr17: 49910422 | DLX4 | Distal Intergenic |
| chr17: 49910427 | DLX4 | Distal Intergenic |
| chr17: 49910431 | DLX4 | Distal Intergenic |
| chr17: 59838380 | VMP1 | Promoter (<=1kb) |
| chr17: 63497324 | ACE | Promoter (1-2kb) |
| chr17: 63497336 | ACE | Promoter (1-2kb) |
| chr17: 63497337 | ACE | Promoter (1-2kb) |
| chr17: 63497342 | ACE | Promoter (1-2kb) |
| chr17: 63497343 | ACE | Promoter (1-2kb) |
| chr17: 63527046 | KCNH6 | Intron (ENST00000583465.1/81033, intron 2 of 12) |
| chr17: 72117887 | SOX9-AS1 | Promoter (<=1kb) |
| chr17: 72117889 | SOX9-AS1 | Promoter (<=1kb) |
| chr17: 72117911 | SOX9-AS1 | Promoter (<=1kb) |
| chr17: 72117945 | SOX9-AS1 | Promoter (<=1kb) |
| chr17: 72117980 | SOX9-AS1 | Promoter (<=1kb) |
| chr17: 72117988 | SOX9-AS1 | Promoter (<=1kb) |
| chr17: 72117989 | SOX9-AS1 | Promoter (<=1kb) |
| chr17: 72118013 | SOX9-AS1 | Promoter (<=1kb) |
| chr17: 72661504 | LINC00511 | Intron (ENST00000255559.7/201266, intron 7 of 9) |
| chr17: 72661511 | LINC00511 | Intron (ENST00000255559.7/201266, intron 7 of 9) |
| chr17: 74852750 | GRIN2C | Exon (ENST00000584176.1/2905, exon 2 of 9) |
| chr17: 74852761 | GRIN2C | Exon (ENST00000584176.1/2905, exon 2 of 9) |
| chr17: 74852762 | GRIN2C | Exon (ENST00000584176.1/2905, exon 2 of 9) |
| chr17: 74852779 | GRIN2C | Exon (ENST00000584176.1/2905, exon 2 of 9) |
| chr17: 74852780 | GRIN2C | Exon (ENST00000584176.1/2905, exon 2 of 9) |
| chr17: 78231866 | TMEM235 | Promoter (<=1kb) |
| chr17: 78231868 | TMEM235 | Promoter (<=1kb) |
| chr17: 78231879 | TMEM235 | Promoter (<=1kb) |
| chr17: 78231881 | TMEM235 | Promoter (<=1kb) |
| chr17: 78231891 | TMEM235 | Promoter (<=1kb) |
| chr17: 78231897 | TMEM235 | Promoter (<=1kb) |
| chr17: 78232001 | TMEM235 | Promoter (<=1kb) |
| chr17: 78232011 | TMEM235 | Promoter (<=1kb) |
| chr17: 78232038 | TMEM235 | Promoter (<=1kb) |
| chr17: 78232040 | TMEM235 | Promoter (<=1kb) |
| chr17: 78232056 | TMEM235 | Promoter (<=1kb) |
| chr17: 78232108 | TMEM235 | Promoter (<=1kb) |
| chr17: 78232138 | TMEM235 | Promoter (<=1kb) |
| chr17: 78232165 | TMEM235 | Promoter (<=1kb) |
| chr17: 78232167 | TMEM235 | Promoter (<=1kb) |
| chr17: 78232184 | TMEM235 | Promoter (<=1kb) |
| chr17: 78232185 | TMEM235 | Promoter (<=1kb) |
| chr17: 78232190 | TMEM235 | Promoter (<=1kb) |
| chr17: 78232191 | TMEM235 | Promoter (<=1kb) |
| chr17: 78232201 | TMEM235 | Promoter (<=1kb) |
| chr17: 78232209 | TMEM235 | Promoter (<=1kb) |
| chr17: 78232210 | TMEM235 | Promoter (<=1kb) |
| chr17: 78505214 | DNAH17 | Intron (ENST00000389840.6/8632, intron 31 of 80) |
| chr17: 78505227 | DNAH17 | Intron (ENST00000389840.6/8632, intron 31 of 80) |
| chr17: 78505240 | DNAH17 | Intron (ENST00000389840.6/8632, intron 31 of 80) |
| chr17: 78505242 | DNAH17 | Intron (ENST00000389840.6/8632, intron 31 of 80) |
| chr17: 81223460 | CEP131 | Promoter (<=1kb) |
| chr17: 81223477 | CEP131 | Promoter (<=1kb) |
| chr17: 81223481 | CEP131 | Promoter (<=1kb) |
| chr17: 81223483 | CEP131 | Promoter (<=1kb) |
| chr17: 81223599 | CEP131 | Promoter (<=1kb) |
| chr17: 81223604 | CEP131 | Promoter (<=1kb) |
| chr17: 81223609 | CEP131 | Promoter (<=1kb) |
| chr17: 81223613 | CEP131 | Promoter (<=1kb) |
| chr17: 81223619 | CEP131 | Promoter (<=1kb) |
| chr17: 81223659 | CEP131 | Promoter (<=1kb) |
| chr17: 81688133 | HGS | Promoter (1-2kb) |
| chr17: 81688134 | HGS | Promoter (1-2kb) |
| chr17: 81688137 | HGS | Promoter (1-2kb) |
| chr17: 81688138 | HGS | Promoter (1-2kb) |
| chr17: 81688140 | HGS | Promoter (1-2kb) |
| chr17: 81688141 | HGS | Promoter (1-2kb) |
| chr17: 81688149 | HGS | Promoter (1-2kb) |
| chr17: 81688150 | HGS | Promoter (1-2kb) |
| chr17: 81688162 | HGS | Promoter (1-2kb) |
| chr17: 81688163 | HGS | Promoter (1-2kb) |
| chr17: 81688165 | HGS | Promoter (1-2kb) |
| chr17: 81688166 | HGS | Promoter (1-2kb) |
| chr17: 81688171 | HGS | Promoter (1-2kb) |
| chr17: 81688172 | HGS | Promoter (1-2kb) |
| chr17: 81688180 | HGS | Promoter (1-2kb) |
| chr17: 81688181 | HGS | Promoter (1-2kb) |
| chr17: 81688186 | HGS | Promoter (1-2kb) |
| chr17: 81688187 | HGS | Promoter (1-2kb) |
| chr17: 81688188 | HGS | Promoter (1-2kb) |
| chr17: 81688189 | HGS | Promoter (1-2kb) |
| chr17: 81688245 | HGS | Promoter (1-2kb) |
| chr17: 81688273 | HGS | Promoter (2-3kb) |
| chr17: 81688284 | HGS | Promoter (2-3kb) |
| chr17: 81688286 | HGS | Promoter (2-3kb) |
| chr17: 81688315 | HGS | Promoter (2-3kb) |
| chr17: 81688329 | HGS | Promoter (2-3kb) |
| chr17: 81688363 | HGS | Promoter (2-3kb) |
| chr17: 81688372 | HGS | Promoter (2-3kb) |
| chr17: 82236900 | MIR6787 | Promoter (<=1kb) |
| chr17: 82236921 | SLC16A3 | Promoter (<=1kb) |
| chr17: 82236977 | SLC16A3 | Promoter (<=1kb) |
| chr18: 9404635 | TWSG1 | Distal Intergenic |
| chr18: 9404670 | TWSG1 | Distal Intergenic |
| chr18: 9404703 | TWSG1 | Distal Intergenic |
| chr18: 9404721 | TWSG1 | Distal Intergenic |
| chr18: 47246674 | SKOR2 | Promoter (2-3kb) |
| chr18: 47246680 | SKOR2 | Promoter (2-3kb) |
| chr18: 47246692 | SKOR2 | Promoter (2-3kb) |
| chr18: 47246705 | SKOR2 | Promoter (2-3kb) |
| chr18: 57428706 | ONECUT2 | Exon (ENST00000620778.1/ENST00000620778.1, exon 1 of 1) |
| chr18: 57428712 | ONECUT2 | Exon (ENST00000620778.1/ENST00000620778.1, exon 1 of 1) |
| chr18: 57428714 | ONECUT2 | Exon (ENST00000620778.1/ENST00000620778.1, exon 1 of 1) |
| chr18: 57428717 | ONECUT2 | Exon (ENST00000620778.1/ENST00000620778.1, exon 1 of 1) |
| chr18: 57428722 | ONECUT2 | Exon (ENST00000620778.1/ENST00000620778.1, exon 1 of 1) |
| chr18: 57428728 | ONECUT2 | Exon (ENST00000620778.1/ENST00000620778.1, exon 1 of 1) |
| chr18: 57428735 | ONECUT2 | Exon (ENST00000620778.1/ENST00000620778.1, exon 1 of 1) |
| chr18: 57428748 | ONECUT2 | Exon (ENST00000620778.1/ENST00000620778.1, exon 1 of 1) |
| chr18: 57428862 | ONECUT2 | Exon (ENST00000620778.1/ENST00000620778.1, exon 1 of 1) |
| chr18: 57428872 | ONECUT2 | Exon (ENST00000620778.1/ENST00000620778.1, exon 1 of 1) |
| chr18: 57428877 | ONECUT2 | Exon (ENST00000620778.1/ENST00000620778.1, exon 1 of 1) |
| chr18: 57428883 | ONECUT2 | Exon (ENST00000620778.1/ENST00000620778.1, exon 1 of 1) |
| chr18: 57428886 | ONECUT2 | Exon (ENST00000620778.1/ENST00000620778.1, exon 1 of 1) |
| chr18: 57428890 | ONECUT2 | Exon (ENST00000620778.1/ENST00000620778.1, exon 1 of 1) |
| chr18: 57428892 | ONECUT2 | Exon (ENST00000620778.1/ENST00000620778.1, exon 1 of 1) |
| chr18: 57440615 | ONECUT2 | Intron (ENST00000491143.3/9480, intron 1 of 1) |
| chr18: 57440635 | ONECUT2 | Intron (ENST00000491143.3/9480, intron 1 of 1) |
| chr18: 57440640 | ONECUT2 | Intron (ENST00000491143.3/9480, intron 1 of 1) |
| chr18: 57440643 | ONECUT2 | Intron (ENST00000491143.3/9480, intron 1 of 1) |
| chr18: 57440650 | ONECUT2 | Intron (ENST00000491143.3/9480, intron 1 of 1) |
| chr18: 57440663 | ONECUT2 | Intron (ENST00000491143.3/9480, intron 1 of 1) |
| chr18: 57440687 | ONECUT2 | Intron (ENST00000491143.3/9480, intron 1 of 1) |
| chr18: 57440688 | ONECUT2 | Intron (ENST00000491143.3/9480, intron 1 of 1) |
| chr18: 57440689 | ONECUT2 | Intron (ENST00000491143.3/9480, intron 1 of 1) |
| chr18: 57440690 | ONECUT2 | Intron (ENST00000491143.3/9480, intron 1 of 1) |
| chr18: 57440693 | ONECUT2 | Intron (ENST00000491143.3/9480, intron 1 of 1) |
| chr18: 57440694 | ONECUT2 | Intron (ENST00000491143.3/9480, intron 1 of 1) |
| chr18: 57440707 | ONECUT2 | Intron (ENST00000491143.3/9480, intron 1 of 1) |
| chr18: 57440712 | ONECUT2 | Intron (ENST00000491143.3/9480, intron 1 of 1) |
| chr18: 59274104 | RAX | Promoter (<=1kb) |
| chr18: 59274109 | RAX | Promoter (<=1kb) |
| chr18: 59274158 | RAX | Promoter (<=1kb) |
| chr18: 59274159 | RAX | Promoter (<=1kb) |
| chr18: 59274162 | RAX | Promoter (<=1kb) |
| chr18: 59274163 | RAX | Promoter (<=1kb) |
| chr18: 68693301 | TMX3 | Promoter (1-2kb) |
| chr18: 68693328 | TMX3 | Promoter (1-2kb) |
| chr18: 68693329 | TMX3 | Promoter (1-2kb) |
| chr18: 68693347 | TMX3 | Promoter (1-2kb) |
| chr19: 290792 | PLPP2 | Promoter (<=1kb) |
| chr19: 290802 | PLPP2 | Promoter (<=1kb) |
| chr19: 290808 | PLPP2 | Promoter (<=1kb) |
| chr19: 290815 | PLPP2 | Promoter (<=1kb) |
| chr19: 290821 | PLPP2 | Promoter (<=1kb) |
| chr19: 290838 | PLPP2 | Promoter (<=1kb) |
| chr19: 290855 | PLPP2 | Promoter (<=1kb) |
| chr19: 290857 | PLPP2 | Promoter (<=1kb) |
| chr19: 290859 | PLPP2 | Promoter (<=1kb) |
| chr19: 290860 | PLPP2 | Promoter (<=1kb) |
| chr19: 290864 | PLPP2 | Promoter (<=1kb) |
| chr19: 290865 | PLPP2 | Promoter (<=1kb) |
| chr19: 290870 | PLPP2 | Promoter (<=1kb) |
| chr19: 290871 | PLPP2 | Promoter (<=1kb) |
| chr19: 290886 | PLPP2 | Promoter (<=1kb) |
| chr19: 290892 | PLPP2 | Promoter (<=1kb) |
| chr19: 290894 | PLPP2 | Promoter (<=1kb) |
| chr19: 795362 | PTBP1 | Promoter (1-2kb) |
| chr19: 795377 | PTBP1 | Promoter (1-2kb) |
| chr19: 795395 | PTBP1 | Promoter (1-2kb) |
| chr19: 795396 | PTBP1 | Promoter (1-2kb) |
| chr19: 795399 | PTBP1 | Promoter (1-2kb) |
| chr19: 795400 | PTBP1 | Promoter (1-2kb) |
| chr19: 795417 | PTBP1 | Promoter (1-2kb) |
| chr19: 795418 | PTBP1 | Promoter (1-2kb) |
| chr19: 795421 | PTBP1 | Promoter (1-2kb) |
| chr19: 1007560 | TMEM259 | Intron (ENST00000234389.3/116444, intron 3 of 8) |
| chr19: 1007563 | TMEM259 | Intron (ENST00000234389.3/116444, intron 3 of 8) |
| chr19: 1007565 | TMEM259 | Intron (ENST00000234389.3/116444, intron 3 of 8) |
| chr19: 1007606 | TMEM259 | Intron (ENST00000234389.3/116444, intron 3 of 8) |
| chr19: 1007612 | TMEM259 | Intron (ENST00000234389.3/116444, intron 3 of 8) |
| chr19: 1007622 | TMEM259 | Intron (ENST00000234389.3/116444, intron 3 of 8) |
| chr19: 1007624 | TMEM259 | Intron (ENST00000234389.3/116444, intron 3 of 8) |
| chr19: 1429508 | DAZAP1 | Intron (ENST00000336761.10/26528, intron 8 of 12) |
| chr19: 1429513 | DAZAP1 | Intron (ENST00000336761.10/26528, intron 8 of 12) |
| chr19: 1429541 | DAZAP1 | Intron (ENST00000336761.10/26528, intron 8 of 12) |
| chr19: 1429544 | DAZAP1 | Intron (ENST00000336761.10/26528, intron 8 of 12) |
| chr19: 1429554 | DAZAP1 | Intron (ENST00000336761.10/26528, intron 8 of 12) |
| chr19: 2251245 | MIR4321 | Promoter (<=1kb) |
| chr19: 2251266 | MIR4321 | Promoter (<=1kb) |
| chr19: 2251275 | MIR4321 | Promoter (<=1kb) |
| chr19: 2251278 | MIR4321 | Promoter (<=1kb) |
| chr19: 2251301 | MIR4321 | Promoter (<=1kb) |
| chr19: 2251309 | MIR4321 | Promoter (<=1kb) |
| chr19: 2251366 | MIR4321 | Promoter (<=1kb) |
| chr19: 2251374 | MIR4321 | Promoter (<=1kb) |
| chr19: 2251377 | MIR4321 | Promoter (<=1kb) |
| chr19: 2251380 | MIR4321 | Promoter (<=1kb) |
| chr19: 2251383 | MIR4321 | Promoter (<=1kb) |
| chr19: 2251392 | MIR4321 | Promoter (<=1kb) |
| chr19: 2251407 | MIR4321 | Promoter (<=1kb) |
| chr19: 2251409 | MIR4321 | Promoter (<=1kb) |
| chr19: 2251429 | MIR4321 | Promoter (<=1kb) |
| chr19: 2251432 | MIR4321 | Promoter (<=1kb) |
| chr19: 2251439 | MIR4321 | Promoter (<=1kb) |
| chr19: 2251446 | MIR4321 | Promoter (<=1kb) |
| chr19: 2251480 | MIR4321 | Promoter (<=1kb) |
| chr19: 2251489 | MIR4321 | Promoter (<=1kb) |
| chr19: 2251491 | MIR4321 | Promoter (<=1kb) |
| chr19: 2251499 | MIR4321 | Promoter (<=1kb) |
| chr19: 2251501 | MIR4321 | Promoter (<=1kb) |
| chr19: 2252251 | MIR4321 | Promoter (1-2kb) |
| chr19: 2252255 | MIR4321 | Promoter (1-2kb) |
| chr19: 2252264 | MIR4321 | Promoter (1-2kb) |
| chr19: 2252275 | MIR4321 | Promoter (1-2kb) |
| chr19: 2252284 | MIR4321 | Promoter (1-2kb) |
| chr19: 2252286 | MIR4321 | Promoter (1-2kb) |
| chr19: 2252291 | MIR4321 | Promoter (1-2kb) |
| chr19: 2252540 | MIR4321 | Promoter (1-2kb) |
| chr19: 2252542 | MIR4321 | Promoter (1-2kb) |
| chr19: 2252589 | MIR4321 | Promoter (1-2kb) |
| chr19: 2252591 | MIR4321 | Promoter (1-2kb) |
| chr19: 2252607 | MIR4321 | Promoter (1-2kb) |
| chr19: 4449490 | UBXN6 | Promoter (<=1kb) |
| chr19: 4450001 | UBXN6 | Promoter (1-2kb) |
| chr19: 4450012 | UBXN6 | Promoter (1-2kb) |
| chr19: 4450026 | UBXN6 | Promoter (1-2kb) |
| chr19: 17497308 | SLC27A1 | Promoter (1-2kb) |
| chr19: 17497313 | SLC27A1 | Promoter (1-2kb) |
| chr19: 17497321 | SLC27A1 | Promoter (1-2kb) |
| chr19: 17497327 | SLC27A1 | Promoter (1-2kb) |
| chr19: 17497329 | SLC27A1 | Promoter (1-2kb) |
| chr19: 17497333 | SLC27A1 | Promoter (1-2kb) |
| chr19: 33811373 | KCTD15 | Exon (ENST00000590385.2/79047, exon 2 of 2) |
| chr19: 33811375 | KCTD15 | Exon (ENST00000590385.2/79047, exon 2 of 2) |
| chr19: 34668203 | SCGB2B2 | Intron (ENST00000601241.5/284402, intron 1 of 3) |
| chr19: 34668207 | SCGB2B2 | Intron (ENST00000601241.5/284402, intron 1 of 3) |
| chr19: 34668224 | SCGB2B2 | Intron (ENST00000601241.5/284402, intron 1 of 3) |
| chr19: 34668229 | SCGB2B2 | Intron (ENST00000601241.5/284402, intron 1 of 3) |
| chr19: 34668235 | SCGB2B2 | Intron (ENST00000601241.5/284402, intron 1 of 3) |
| chr19: 34668260 | SCGB2B2 | Intron (ENST00000601241.5/284402, intron 1 of 3) |
| chr19: 34668278 | SCGB2B2 | Intron (ENST00000601241.5/284402, intron 1 of 3) |
| chr19: 34668285 | SCGB2B2 | Intron (ENST00000601241.5/284402, intron 1 of 3) |
| chr19: 34668387 | SCGB2B2 | Intron (ENST00000601241.5/284402, intron 1 of 3) |
| chr19: 34668399 | SCGB2B2 | Intron (ENST00000601241.5/284402, intron 1 of 3) |
| chr19: 34668537 | SCGB2B2 | Intron (ENST00000601241.5/284402, intron 1 of 3) |
| chr19: 34668540 | SCGB2B2 | Intron (ENST00000601241.5/284402, intron 1 of 3) |
| chr19: 34668542 | SCGB2B2 | Intron (ENST00000601241.5/284402, intron 1 of 3) |
| chr19: 37007801 | ZNF420 | Promoter (<=1kb) |
| chr19: 37007807 | ZNF420 | Promoter (<=1kb) |
| chr19: 37007809 | ZNF420 | Promoter (<=1kb) |
| chr19: 38291920 | SPINT2 | Promoter (1-2kb) |
| chr19: 38291921 | SPINT2 | Promoter (1-2kb) |
| chr19: 38291935 | SPINT2 | Promoter (1-2kb) |
| chr19: 38291936 | SPINT2 | Promoter (1-2kb) |
| chr19: 38291945 | SPINT2 | Promoter (1-2kb) |
| chr19: 38291950 | SPINT2 | Promoter (1-2kb) |
| chr19: 38395561 | SPRED3 | Exon (ENST00000338502.8/399473, exon 5 of 5) |
| chr19: 38395565 | SPRED3 | Exon (ENST00000338502.8/399473, exon 5 of 5) |
| chr19: 38395567 | SPRED3 | Exon (ENST00000338502.8/399473, exon 5 of 5) |
| chr19: 38395573 | SPRED3 | Exon (ENST00000338502.8/399473, exon 5 of 5) |
| chr19: 38395608 | SPRED3 | Exon (ENST00000338502.8/399473, exon 5 of 5) |
| chr19: 38395609 | SPRED3 | Exon (ENST00000338502.8/399473, exon 5 of 5) |
| chr19: 38395623 | SPRED3 | Exon (ENST00000338502.8/399473, exon 5 of 5) |
| chr19: 38395624 | SPRED3 | Exon (ENST00000338502.8/399473, exon 5 of 5) |
| chr19: 38395626 | SPRED3 | Exon (ENST00000338502.8/399473, exon 5 of 5) |
| chr19: 38395627 | SPRED3 | Exon (ENST00000338502.8/399473, exon 5 of 5) |
| chr19: 38395891 | SPRED3 | Exon (ENST00000338502.8/399473, exon 5 of 5) |
| chr19: 38395894 | SPRED3 | Exon (ENST00000338502.8/399473, exon 5 of 5) |
| chr19: 38395914 | SPRED3 | Exon (ENST00000338502.8/399473, exon 5 of 5) |
| chr19: 38395917 | SPRED3 | Exon (ENST00000338502.8/399473, exon 5 of 5) |
| chr19: 38395944 | SPRED3 | Exon (ENST00000338502.8/399473, exon 5 of 5) |
| chr19: 38395948 | SPRED3 | Exon (ENST00000338502.8/399473, exon 5 of 5) |
| chr19: 38395951 | SPRED3 | Exon (ENST00000338502.8/399473, exon 5 of 5) |
| chr19: 38395965 | SPRED3 | Exon (ENST00000338502.8/399473, exon 5 of 5) |
| chr19: 38395971 | SPRED3 | Exon (ENST00000338502.8/399473, exon 5 of 5) |
| chr19: 38395975 | SPRED3 | Exon (ENST00000338502.8/399473, exon 5 of 5) |
| chr19: 38395981 | SPRED3 | Exon (ENST00000338502.8/399473, exon 5 of 5) |
| chr19: 38395994 | SPRED3 | Exon (ENST00000338502.8/399473, exon 5 of 5) |
| chr19: 38395996 | SPRED3 | Exon (ENST00000338502.8/399473, exon 5 of 5) |
| chr19: 38396001 | SPRED3 | Exon (ENST00000338502.8/399473, exon 5 of 5) |
| chr19: 38396004 | SPRED3 | Exon (ENST00000338502.8/399473, exon 5 of 5) |
| chr19: 38396006 | SPRED3 | Exon (ENST00000338502.8/399473, exon 5 of 5) |
| chr19: 38396008 | SPRED3 | Exon (ENST00000338502.8/399473, exon 5 of 5) |
| chr19: 38396016 | SPRED3 | Exon (ENST00000338502.8/399473, exon 5 of 5) |
| chr19: 40567965 | SPTBN4 | 3′ UTR |
| chr19: 40567967 | SPTBN4 | 3′ UTR |
| chr19: 40567970 | SPTBN4 | 3′ UTR |
| chr19: 40567971 | SPTBN4 | 3′ UTR |
| chr19: 40567980 | SPTBN4 | 3′ UTR |
| chr19: 40567981 | SPTBN4 | 3′ UTR |
| chr19: 40567982 | SPTBN4 | 3′ UTR |
| chr19: 40567983 | SPTBN4 | 3′ UTR |
| chr19: 40567989 | SPTBN4 | 3′ UTR |
| chr19: 40567990 | SPTBN4 | 3′ UTR |
| chr19: 40567991 | SPTBN4 | 3′ UTR |
| chr19: 40567992 | SPTBN4 | 3′ UTR |
| chr19: 40567994 | SPTBN4 | 3′ UTR |
| chr19: 40567995 | SPTBN4 | 3′ UTR |
| chr19: 40567997 | SPTBN4 | 3′ UTR |
| chr19: 40567998 | SPTBN4 | 3′ UTR |
| chr19: 40568009 | SPTBN4 | 3′ UTR |
| chr19: 40597053 | LTBP4 | Promoter (<=1kb) |
| chr19: 40597054 | LTBP4 | Promoter (<=1kb) |
| chr19: 40597055 | LTBP4 | Promoter (<=1kb) |
| chr19: 40597056 | LTBP4 | Promoter (<=1kb) |
| chr19: 40597057 | LTBP4 | Promoter (<=1kb) |
| chr19: 40597058 | LTBP4 | Promoter (<=1kb) |
| chr19: 40597060 | LTBP4 | Promoter (<=1kb) |
| chr19: 40597061 | LTBP4 | Promoter (<=1kb) |
| chr19: 40597064 | LTBP4 | Promoter (<=1kb) |
| chr19: 40597065 | LTBP4 | Promoter (<=1kb) |
| chr19: 40597070 | LTBP4 | Promoter (<=1kb) |
| chr19: 40597071 | LTBP4 | Promoter (<=1kb) |
| chr19: 40597074 | LTBP4 | Promoter (<=1kb) |
| chr19: 40597134 | LTBP4 | Promoter (<=1kb) |
| chr19: 40597140 | LTBP4 | Promoter (<=1kb) |
| chr19: 40597144 | LTBP4 | Promoter (<=1kb) |
| chr19: 40597150 | LTBP4 | Promoter (<=1kb) |
| chr19: 40597156 | LTBP4 | Promoter (<=1kb) |
| chr19: 40597168 | LTBP4 | Promoter (<=1kb) |
| chr19: 40597190 | LTBP4 | Promoter (<=1kb) |
| chr19: 40597202 | LTBP4 | Promoter (<=1kb) |
| chr19: 40597206 | LTBP4 | Promoter (<=1kb) |
| chr19: 40597214 | LTBP4 | Promoter (<=1kb) |
| chr19: 40597221 | LTBP4 | Promoter (<=1kb) |
| chr19: 40597224 | LTBP4 | Promoter (<=1kb) |
| chr19: 40597228 | LTBP4 | Promoter (<=1kb) |
| chr19: 40597233 | LTBP4 | Promoter (<=1kb) |
| chr19: 40597239 | LTBP4 | Promoter (<=1kb) |
| chr19: 40597255 | LTBP4 | Promoter (<=1kb) |
| chr19: 40609554 | LTBP4 | Promoter (<=1kb) |
| chr19: 40609570 | LTBP4 | Promoter (<=1kb) |
| chr19: 40609571 | LTBP4 | Promoter (<=1kb) |
| chr19: 40609580 | LTBP4 | Promoter (<=1kb) |
| chr19: 40609581 | LTBP4 | Promoter (<=1kb) |
| chr19: 40609600 | LTBP4 | Promoter (<=1kb) |
| chr19: 40609601 | LTBP4 | Promoter (<=1kb) |
| chr19: 40609612 | LTBP4 | Promoter (<=1kb) |
| chr19: 40609613 | LTBP4 | Promoter (<=1kb) |
| chr19: 40609618 | LTBP4 | Promoter (<=1kb) |
| chr19: 40609619 | LTBP4 | Promoter (<=1kb) |
| chr19: 40609754 | LTBP4 | Promoter (<=1kb) |
| chr19: 40609764 | LTBP4 | Promoter (<=1kb) |
| chr19: 40609766 | LTBP4 | Promoter (<=1kb) |
| chr19: 40609774 | LTBP4 | Promoter (<=1kb) |
| chr19: 40609790 | LTBP4 | Promoter (<=1kb) |
| chr19: 40609791 | LTBP4 | Promoter (<=1kb) |
| chr19: 40609795 | LTBP4 | Promoter (<=1kb) |
| chr19: 40609796 | LTBP4 | Promoter (<=1kb) |
| chr19: 40609817 | LTBP4 | Promoter (<=1kb) |
| chr19: 40609818 | LTBP4 | Promoter (<=1kb) |
| chr19: 40609822 | LTBP4 | Promoter (<=1kb) |
| chr19: 40609823 | LTBP4 | Promoter (<=1kb) |
| chr19: 40609828 | LTBP4 | Promoter (<=1kb) |
| chr19: 40609829 | LTBP4 | Promoter (<=1kb) |
| chr19: 40614332 | LTBP4 | Promoter (<=1kb) |
| chr19: 40614336 | LTBP4 | Promoter (<=1kb) |
| chr19: 40614353 | LTBP4 | Promoter (<=1kb) |
| chr19: 40614358 | LTBP4 | Promoter (<=1kb) |
| chr19: 40614363 | LTBP4 | Promoter (<=1kb) |
| chr19: 40623976 | LTBP4 | Promoter (<=1kb) |
| chr19: 40623980 | LTBP4 | Promoter (<=1kb) |
| chr19: 41904348 | ARHGEF1 | Promoter (<=1kb) |
| chr19: 41904359 | ARHGEF1 | Promoter (<=1kb) |
| chr19: 41904369 | ARHGEF1 | Promoter (<=1kb) |
| chr19: 41904371 | ARHGEF1 | Promoter (<=1kb) |
| chr19: 50506480 | JOSD2 | Promoter (1-2kb) |
| chr19: 50506485 | JOSD2 | Promoter (1-2kb) |
| chr19: 50506506 | JOSD2 | Promoter (1-2kb) |
| chr19: 50506511 | JOSD2 | Promoter (1-2kb) |
| chr19: 50506515 | JOSD2 | Promoter (1-2kb) |
| chr19: 53982494 | MIR935 | Promoter (<=1kb) |
| chr19: 53982504 | MIR935 | Promoter (<=1kb) |
| chr19: 53982505 | MIR935 | Promoter (<=1kb) |
| chr19: 53982523 | MIR935 | Promoter (<=1kb) |
| chr19: 53982606 | MIR935 | Promoter (<=1kb) |
| chr19: 53982609 | MIR935 | Promoter (<=1kb) |
| chr19: 53982612 | MIR935 | Promoter (<=1kb) |
| chr19: 53982621 | MIR935 | Promoter (<=1kb) |
| chr19: 53982681 | MIR935 | Promoter (<=1kb) |
| chr19: 53982682 | MIR935 | Promoter (<=1kb) |
| chr19: 53982698 | MIR935 | Promoter (<=1kb) |
| chr19: 53982699 | MIR935 | Promoter (<=1kb) |
| chr19: 53982702 | MIR935 | Promoter (<=1kb) |
| chr19: 53982705 | MIR935 | Promoter (<=1kb) |
| chr19: 53982708 | MIR935 | Promoter (<=1kb) |
| chr19: 53982709 | MIR935 | Promoter (<=1kb) |
| chr19: 53982714 | MIR935 | Promoter (<=1kb) |
| chr19: 53982720 | MIR935 | Promoter (<=1kb) |
| chr19: 53982735 | MIR935 | Promoter (<=1kb) |
| chr19: 53982736 | MIR935 | Promoter (<=1kb) |
| chr19: 53982740 | MIR935 | Promoter (<=1kb) |
| chr19: 53982741 | MIR935 | Promoter (<=1kb) |
| chr19: 53982744 | MIR935 | Promoter (<=1kb) |
| chr19: 53982747 | MIR935 | Promoter (<=1kb) |
| chr19: 53982751 | MIR935 | Promoter (<=1kb) |
| chr19: 53982752 | MIR935 | Promoter (<=1kb) |
| chr19: 53982753 | MIR935 | Promoter (<=1kb) |
| chr19: 53982760 | MIR935 | Promoter (<=1kb) |
| chr19: 53982770 | MIR935 | Promoter (<=1kb) |
| chr19: 53982875 | MIR935 | Promoter (<=1kb) |
| chr19: 57935179 | ZNF418 | Promoter (<=1kb) |
| chr19: 57935180 | ZNF418 | Promoter (<=1kb) |
| chr19: 57935181 | ZNF418 | Promoter (<=1kb) |
| chr19: 57935186 | ZNF418 | Promoter (<=1kb) |
| chr19: 57935188 | ZNF418 | Promoter (<=1kb) |
| chr19: 57935189 | ZNF418 | Promoter (<=1kb) |
| chr19: 57935202 | ZNF418 | Promoter (<=1kb) |
| chr19: 57935203 | ZNF418 | Promoter (<=1kb) |
| chr19: 57935204 | ZNF418 | Promoter (<=1kb) |
| chr19: 57935205 | ZNF418 | Promoter (<=1kb) |
| chr2: 10043082 | KLF11 | Promoter (<=1kb) |
| chr2: 10043088 | KLF11 | Promoter (<=1kb) |
| chr2: 42101596 | PKDCC | Distal Intergenic |
| chr2: 42101598 | PKDCC | Distal Intergenic |
| chr2: 42101601 | PKDCC | Distal Intergenic |
| chr2: 42101625 | PKDCC | Distal Intergenic |
| chr2: 42101627 | PKDCC | Distal Intergenic |
| chr2: 42101633 | PKDCC | Distal Intergenic |
| chr2: 43224454 | ZFP36L2 | Promoter (2-3kb) |
| chr2: 43224469 | ZFP36L2 | Promoter (2-3kb) |
| chr2: 43224471 | ZFP36L2 | Promoter (2-3kb) |
| chr2: 43224474 | ZFP36L2 | Promoter (2-3kb) |
| chr2: 43224480 | ZFP36L2 | Promoter (2-3kb) |
| chr2: 43224484 | ZFP36L2 | Promoter (2-3kb) |
| chr2: 43224489 | ZFP36L2 | Promoter (2-3kb) |
| chr2: 43224497 | ZFP36L2 | Promoter (2-3kb) |
| chr2: 43224500 | ZFP36L2 | Promoter (2-3kb) |
| chr2: 43224502 | ZFP36L2 | Promoter (2-3kb) |
| chr2: 43225009 | ZFP36L2 | Promoter (1-2kb) |
| chr2: 43225012 | ZFP36L2 | Promoter (1-2kb) |
| chr2: 43225021 | ZFP36L2 | Promoter (1-2kb) |
| chr2: 43225082 | ZFP36L2 | Promoter (1-2kb) |
| chr2: 43225087 | ZFP36L2 | Promoter (1-2kb) |
| chr2: 43225089 | ZFP36L2 | Promoter (1-2kb) |
| chr2: 43225091 | ZFP36L2 | Promoter (1-2kb) |
| chr2: 44929769 | LINC01833 | Intron (ENST00000437916.2/107985879, intron 1 of 1) |
| chr2: 44929799 | LINC01833 | Intron (ENST00000437916.2/107985879, intron 1 of 1) |
| chr2: 44929802 | LINC01833 | Intron (ENST00000437916.2/107985879, intron 1 of 1) |
| chr2: 64609327 | LINC02579 | Promoter (1-2kb) |
| chr2: 64609338 | LINC02579 | Promoter (2-3kb) |
| chr2: 64609342 | LINC02579 | Promoter (2-3kb) |
| chr2: 64609368 | LINC02579 | Promoter (2-3kb) |
| chr2: 64609373 | LINC02579 | Promoter (2-3kb) |
| chr2: 64609383 | LINC02579 | Promoter (2-3kb) |
| chr2: 64609403 | LINC02579 | Promoter (2-3kb) |
| chr2: 64609404 | LINC02579 | Promoter (2-3kb) |
| chr2: 64609407 | LINC02579 | Promoter (2-3kb) |
| chr2: 64609408 | LINC02579 | Promoter (2-3kb) |
| chr2: 74499366 | LBX2 | Promoter (<=1kb) |
| chr2: 74499368 | LBX2 | Promoter (<=1kb) |
| chr2: 74499398 | LBX2 | Promoter (<=1kb) |
| chr2: 74499400 | LBX2 | Promoter (<=1kb) |
| chr2: 74499407 | LBX2 | Promoter (<=1kb) |
| chr2: 74499410 | LBX2 | Promoter (<=1kb) |
| chr2: 74499413 | LBX2 | Promoter (<=1kb) |
| chr2: 74499427 | LBX2 | Promoter (<=1kb) |
| chr2: 74499442 | LBX2 | Promoter (<=1kb) |
| chr2: 74499445 | LBX2 | Promoter (<=1kb) |
| chr2: 74499458 | LBX2 | Promoter (<=1kb) |
| chr2: 74499470 | LBX2 | Promoter (<=1kb) |
| chr2: 74499811 | LBX2 | Promoter (<=1kb) |
| chr2: 74499822 | LBX2 | Promoter (<=1kb) |
| chr2: 74499824 | LBX2 | Promoter (<=1kb) |
| chr2: 74499829 | LBX2 | Promoter (<=1kb) |
| chr2: 74499834 | LBX2 | Promoter (<=1kb) |
| chr2: 74499842 | LBX2 | Promoter (<=1kb) |
| chr2: 98539614 | INPP4A | Exon (ENST00000409016.8/3631, exon 10 of 26) |
| chr2: 98539629 | INPP4A | Exon (ENST00000409016.8/3631, exon 10 of 26) |
| chr2: 106065547 | ECRG4 | Promoter (<=1kb) |
| chr2: 106065562 | ECRG4 | Promoter (<=1kb) |
| chr2: 106065572 | ECRG4 | Promoter (<=1kb) |
| chr2: 106065576 | ECRG4 | Promoter (<=1kb) |
| chr2: 106065580 | ECRG4 | Promoter (<=1kb) |
| chr2: 106065587 | ECRG4 | Promoter (<=1kb) |
| chr2: 106065616 | ECRG4 | Promoter (<=1kb) |
| chr2: 106065618 | ECRG4 | Promoter (<=1kb) |
| chr2: 106065621 | ECRG4 | Promoter (<=1kb) |
| chr2: 106065629 | ECRG4 | Promoter (<=1kb) |
| chr2: 106065639 | ECRG4 | Promoter (<=1kb) |
| chr2: 106065652 | ECRG4 | Promoter (<=1kb) |
| chr2: 106065665 | ECRG4 | Promoter (<=1kb) |
| chr2: 106065666 | ECRG4 | Promoter (<=1kb) |
| chr2: 106065667 | ECRG4 | Promoter (<=1kb) |
| chr2: 106065668 | ECRG4 | Promoter (<=1kb) |
| chr2: 106065671 | ECRG4 | Promoter (<=1kb) |
| chr2: 106065672 | ECRG4 | Promoter (<=1kb) |
| chr2: 106065673 | ECRG4 | Promoter (<=1kb) |
| chr2: 106065674 | ECRG4 | Promoter (<=1kb) |
| chr2: 106065681 | ECRG4 | Promoter (<=1kb) |
| chr2: 106065682 | ECRG4 | Promoter (<=1kb) |
| chr2: 106065685 | ECRG4 | Promoter (<=1kb) |
| chr2: 106065686 | ECRG4 | Promoter (<=1kb) |
| chr2: 106065699 | ECRG4 | Promoter (<=1kb) |
| chr2: 106065701 | ECRG4 | Promoter (<=1kb) |
| chr2: 106065715 | ECRG4 | Promoter (<=1kb) |
| chr2: 106065725 | ECRG4 | Promoter (<=1kb) |
| chr2: 106065732 | ECRG4 | Promoter (<=1kb) |
| chr2: 106065739 | ECRG4 | Promoter (<=1kb) |
| chr2: 106065751 | ECRG4 | Promoter (<=1kb) |
| chr2: 106065782 | ECRG4 | Promoter (<=1kb) |
| chr2: 106065784 | ECRG4 | Promoter (<=1kb) |
| chr2: 106065814 | ECRG4 | Promoter (<=1kb) |
| chr2: 106065849 | ECRG4 | Promoter (<=1kb) |
| chr2: 106065925 | ECRG4 | Promoter (<=1kb) |
| chr2: 106065929 | ECRG4 | Promoter (<=1kb) |
| chr2: 106065943 | ECRG4 | Promoter (<=1kb) |
| chr2: 106065946 | ECRG4 | Promoter (<=1kb) |
| chr2: 120887649 | GLI2 | Intron (ENST00000341310.10/2736, intron 2 of 9) |
| chr2: 120887681 | GLI2 | Intron (ENST00000341310.10/2736, intron 2 of 9) |
| chr2: 176082272 | EVX2 | Promoter (1-2kb) |
| chr2: 176082274 | EVX2 | Promoter (1-2kb) |
| chr2: 176082276 | EVX2 | Promoter (1-2kb) |
| chr2: 176082285 | EVX2 | Promoter (1-2kb) |
| chr2: 176082287 | EVX2 | Promoter (1-2kb) |
| chr2: 176082288 | EVX2 | Promoter (1-2kb) |
| chr2: 176082295 | EVX2 | Promoter (1-2kb) |
| chr2: 176082296 | EVX2 | Promoter (1-2kb) |
| chr2: 176082298 | EVX2 | Promoter (1-2kb) |
| chr2: 176082299 | EVX2 | Promoter (1-2kb) |
| chr2: 176082346 | EVX2 | Promoter (1-2kb) |
| chr2: 176082349 | EVX2 | Promoter (1-2kb) |
| chr2: 176082351 | EVX2 | Promoter (1-2kb) |
| chr2: 176082357 | EVX2 | Promoter (1-2kb) |
| chr2: 176082360 | EVX2 | Promoter (1-2kb) |
| chr2: 176082369 | EVX2 | Promoter (1-2kb) |
| chr2: 176082391 | EVX2 | Promoter (1-2kb) |
| chr2: 176082404 | EVX2 | Promoter (1-2kb) |
| chr2: 176082410 | EVX2 | Promoter (1-2kb) |
| chr2: 176082413 | EVX2 | Promoter (1-2kb) |
| chr2: 176082416 | EVX2 | Promoter (1-2kb) |
| chr2: 176082430 | EVX2 | Promoter (1-2kb) |
| chr2: 176082435 | EVX2 | Promoter (1-2kb) |
| chr2: 176082444 | EVX2 | Promoter (1-2kb) |
| chr2: 176082462 | EVX2 | Promoter (1-2kb) |
| chr2: 176082463 | EVX2 | Promoter (1-2kb) |
| chr2: 176082472 | EVX2 | Promoter (1-2kb) |
| chr2: 176082473 | EVX2 | Promoter (1-2kb) |
| chr2: 176082503 | EVX2 | Promoter (1-2kb) |
| chr2: 176082514 | EVX2 | Promoter (1-2kb) |
| chr2: 176082516 | EVX2 | Promoter (1-2kb) |
| chr2: 176082518 | EVX2 | Promoter (1-2kb) |
| chr2: 176082523 | EVX2 | Promoter (1-2kb) |
| chr2: 176082534 | EVX2 | Promoter (1-2kb) |
| chr2: 205885781 | NRP2 | Distal Intergenic |
| chr2: 205885792 | NRP2 | Distal Intergenic |
| chr2: 205885798 | NRP2 | Distal Intergenic |
| chr2: 217978608 | LOC105373878 | Promoter (<=1kb) |
| chr2: 217978612 | LOC105373878 | Promoter (<=1kb) |
| chr2: 217978628 | LOC105373878 | Promoter (<=1kb) |
| chr2: 219496796 | SPEGNB | Promoter (<=1kb) |
| chr2: 219496801 | SPEGNB | Promoter (<=1kb) |
| chr2: 219496803 | SPEGNB | Promoter (<=1kb) |
| chr2: 219496825 | SPEGNB | Promoter (<=1kb) |
| chr2: 219496827 | SPEGNB | Promoter (<=1kb) |
| chr2: 219496838 | SPEGNB | Promoter (<=1kb) |
| chr2: 219496841 | SPEGNB | Promoter (<=1kb) |
| chr2: 219496843 | SPEGNB | Promoter (<=1kb) |
| chr2: 219496856 | SPEGNB | Promoter (<=1kb) |
| chr2: 219496879 | SPEGNB | Promoter (<=1kb) |
| chr2: 219496888 | SPEGNB | Promoter (<=1kb) |
| chr2: 219496890 | SPEGNB | Promoter (<=1kb) |
| chr2: 219522719 | ASIC4 | Intron (ENST00000461395.5/55515, intron 1 of 2) |
| chr2: 219522725 | ASIC4 | Intron (ENST00000461395.5/55515, intron 1 of 2) |
| chr2: 219522728 | ASIC4 | Intron (ENST00000461395.5/55515, intron 1 of 2) |
| chr2: 219522742 | ASIC4 | Intron (ENST00000461395.5/55515, intron 1 of 2) |
| chr2: 219522747 | ASIC4 | Intron (ENST00000461395.5/55515, intron 1 of 2) |
| chr2: 219522753 | ASIC4 | Intron (ENST00000461395.5/55515, intron 1 of 2) |
| chr2: 219522757 | ASIC4 | Intron (ENST00000461395.5/55515, intron 1 of 2) |
| chr2: 219522760 | ASIC4 | Intron (ENST00000461395.5/55515, intron 1 of 2) |
| chr2: 219522773 | ASIC4 | Intron (ENST00000461395.5/55515, intron 1 of 2) |
| chr2: 219522775 | ASIC4 | Intron (ENST00000461395.5/55515, intron 1 of 2) |
| chr20: 21391640 | NKX2-4 | Distal Intergenic |
| chr20: 21391647 | NKX2-4 | Distal Intergenic |
| chr20: 21391656 | NKX2-4 | Distal Intergenic |
| chr20: 21391658 | NKX2-4 | Distal Intergenic |
| chr20: 37915006 | VSTM2L | Intron (ENST00000373459.4/128434, intron 1 of 1) |
| chr20: 37915009 | VSTM2L | Intron (ENST00000373459.4/128434, intron 1 of 1) |
| chr20: 37915027 | VSTM2L | Intron (ENST00000373459.4/128434, intron 1 of 1) |
| chr20: 37915028 | VSTM2L | Intron (ENST00000373459.4/128434, intron 1 of 1) |
| chr20: 37915046 | VSTM2L | Intron (ENST00000373459.4/128434, intron 1 of 1) |
| chr20: 37915053 | VSTM2L | Intron (ENST00000373459.4/128434, intron 1 of 1) |
| chr20: 37915056 | VSTM2L | Intron (ENST00000373459.4/128434, intron 1 of 1) |

### Example 5-Additional Methylation Markers for SCLC Subtype Classification, Diagnosis, and Treatment

Additional methylation sites associated with each SCLC subtype, SCLC-A. SCLC-N, SCLC-P, and SCLC-I were identified from analysis of methylation data from formalin-fixed paraffin-embedded (FFPE) tissue sections from SCLC patients. Methylation sites associated with SCLC-A are provided in Table 20. Methylation sites associated with SCLC-N are provided in Table 21. Methylation sites associated with SCLC-P are provided in Table 22. Methylation sites associated with SCLC-I are provided in Table 23.

TABLE 20
Additional methylation sites associated with the SCLC-A subtype

| Methylation Site | Transcript_ID | Gene_Symbol | Gene_Region | Subtype |
|---|---|---|---|---|
| chr1: 958378 | ENST00000477976.5 | NOC2L | Exon | SCLC-A |
| chr1: 1348250 | ENST00000378891.9 | DVL1 | Intron | SCLC-A |
| chr1: 3524391 | ENST00000356575.9 | MEGF6 | Intron | SCLC-A |
| chr1: 3524397 | ENST00000356575.9 | MEGF6 | Intron | SCLC-A |
| chr1: 3704686 | ENST00000378295.9 | TP73 | Intron | SCLC-A |
| chr1: 4734864 | ENST00000378191.5 | AJAP1 | Intron | SCLC-A |
| chr1: 5493929 | ENST00000564261.1 | NA | Exon | SCLC-A |
| chr1: 5493969 | ENST00000564261.1 | NA | Exon | SCLC-A |
| chr1: 6378290 | ENST00000608083.5 | ACOT7 | Intron | SCLC-A |
| chr1: 6378309 | ENST00000608083.5 | ACOT7 | Intron | SCLC-A |
| chr1: 6378318 | ENST00000608083.5 | ACOT7 | Intron | SCLC-A |
| chr1: 6378319 | ENST00000608083.5 | ACOT7 | Intron | SCLC-A |
| chr1: 7616559 | ENST00000303635.12 | CAMTA1 | Intron | SCLC-A |
| chr1: 7649286 | ENST00000303635.12 | CAMTA1 | Intron | SCLC-A |
| chr1: 8680968 | ENST00000656437.1 | RERE | Intron | SCLC-A |
| chr1: 8681121 | ENST00000656437.1 | RERE | Intron | SCLC-A |
| chr1: 10643731 | ENST00000606802.1 | NA | Intron | SCLC-A |
| chr1: 10643800 | ENST00000606802.1 | NA | Intron | SCLC-A |
| chr1: 10643801 | ENST00000606802.1 | NA | Intron | SCLC-A |
| chr1: 10643812 | ENST00000606802.1 | NA | Intron | SCLC-A |
| chr1: 10658338 | ENST00000377022.8 | CASZ1 | Intron | SCLC-A |
| chr1: 10658341 | ENST00000377022.8 | CASZ1 | Intron | SCLC-A |
| chr1: 10658374 | ENST00000377022.8 | CASZ1 | Intron | SCLC-A |
| chr1: 10658375 | ENST00000377022.8 | CASZ1 | Intron | SCLC-A |
| chr1: 10658433 | ENST00000377022.8 | CASZ1 | Intron | SCLC-A |
| chr1: 10658451 | ENST00000377022.8 | CASZ1 | Intron | SCLC-A |
| chr1: 10658452 | ENST00000377022.8 | CASZ1 | Intron | SCLC-A |
| chr1: 10658548 | ENST00000492173.1 | CASZ1 | Exon | SCLC-A |
| chr1: 10658572 | ENST00000492173.1 | CASZ1 | Exon | SCLC-A |
| chr1: 10658839 | ENST00000377022.8 | CASZ1 | Intron | SCLC-A |
| chr1: 10658845 | ENST00000377022.8 | CASZ1 | Intron | SCLC-A |
| chr1: 10704614 | ENST00000377022.8 | CASZ1 | Intron | SCLC-A |
| chr1: 10948170 | ENST00000476357.1 | C1orf127 | Exon | SCLC-A |
| chr1: 10948349 | ENST00000476357.1 | C1orf127 | Exon | SCLC-A |
| chr1: 10948642 | ENST00000476357.1 | C1orf127 | Exon | SCLC-A |
| chr1: 11484596 | ENST00000294484.7 | DISP3 | Intron | SCLC-A |
| chr1: 15735048 | ENST00000453804.1 | NA | Intron | SCLC-A |
| chr1: 15735060 | ENST00000453804.1 | NA | Intron | SCLC-A |
| chr1: 15765708 | ENST00000502739.5 | FBLIM1 | Intron | SCLC-A |
| chr1: 15765709 | ENST00000502739.5 | FBLIM1 | Intron | SCLC-A |
| chr1: 15765718 | ENST00000502739.5 | FBLIM1 | Intron | SCLC-A |
| chr1: 15765815 | ENST00000502739.5 | FBLIM1 | Intron | SCLC-A |
| chr1: 15941962 | ENST00000462525.1 | ZBTB17 | Exon | SCLC-A |
| chr1: 15942041 | ENST00000462525.1 | ZBTB17 | Exon | SCLC-A |
| chr1: 15942046 | ENST00000462525.1 | ZBTB17 | Exon | SCLC-A |
| chr1: 15942052 | ENST00000462525.1 | ZBTB17 | Exon | SCLC-A |
| chr1: 15942086 | ENST00000462525.1 | ZBTB17 | Exon | SCLC-A |
| chr1: 18155744 | ENST00000251296.4 | IGSF21 | Intron | SCLC-A |
| chr1: 18190698 | ENST00000251296.4 | IGSF21 | Intron | SCLC-A |
| chr1: 18190842 | ENST00000251296.4 | IGSF21 | Intron | SCLC-A |
| chr1: 18265745 | ENST00000251296.4 | IGSF21 | Intron | SCLC-A |
| chr1: 18282300 | ENST00000251296.4 | IGSF21 | Intron | SCLC-A |
| chr1: 18482634 | ENST00000400664.2 | KLHDC7A | Exon | SCLC-A |
| chr1: 18482653 | ENST00000400664.2 | KLHDC7A | Exon | SCLC-A |
| chr1: 18482654 | ENST00000400664.2 | KLHDC7A | Exon | SCLC-A |
| chr1: 18482655 | ENST00000400664.2 | KLHDC7A | Exon | SCLC-A |
| chr1: 18482656 | ENST00000400664.2 | KLHDC7A | Exon | SCLC-A |
| chr1: 18482672 | ENST00000400664.2 | KLHDC7A | Exon | SCLC-A |
| chr1: 18482678 | ENST00000400664.2 | KLHDC7A | Exon | SCLC-A |
| chr1: 18482679 | ENST00000400664.2 | KLHDC7A | Exon | SCLC-A |
| chr1: 18482691 | ENST00000400664.2 | KLHDC7A | Exon | SCLC-A |
| chr1: 18482702 | ENST00000400664.2 | KLHDC7A | Exon | SCLC-A |
| chr1: 21588078 | ENST00000457706.1 | NA | Intron | SCLC-A |
| chr1: 21603793 | ENST00000611549.4 | RAP1GAP | Exon | SCLC-A |
| chr1: 22853986 | ENST00000544305.5 | EPHB2 | Intron | SCLC-A |
| chr1: 22853987 | ENST00000544305.5 | EPHB2 | Intron | SCLC-A |
| chr1: 25771820 | ENST00000496532.5 | MAN1C1 | Promoter | SCLC-A |
| chr1: 25771857 | ENST00000496532.5 | MAN1C1 | Promoter | SCLC-A |
| chr1: 25771872 | ENST00000496532.5 | MAN1C1 | Promoter | SCLC-A |
| chr1: 25771902 | ENST00000496532.5 | MAN1C1 | Promoter | SCLC-A |
| chr1: 25810408 | ENST00000354177.8 | SELENON | Intron | SCLC-A |
| chr1: 26287066 | ENST00000314675.11 | UBXN11 | Intron | SCLC-A |
| chr1: 26287108 | ENST00000314675.11 | UBXN11 | Intron | SCLC-A |
| chr1: 26856854 | ENST00000374142.9 | ZDHHC18 | Exon | SCLC-A |
| chr1: 28815683 | ENST00000621425.1 | OPRD1 | Intron | SCLC-A |
| chr1: 28815743 | ENST00000621425.1 | OPRD1 | Intron | SCLC-A |
| chr1: 28869196 | ENST00000234961.7 | OPRD1 | Exon | SCLC-A |
| chr1: 31626876 | ENST00000373705.1 | HCRTR1 | Intron | SCLC-A |
| chr1: 36846189 | ENST00000373091.8 | GRIK3 | Intron | SCLC-A |
| chr1: 38004855 | ENST00000485803.5 | FHL3 | Intron | SCLC-A |
| chr1: 38064302 | ENST00000432922.5 | NA | Intron | SCLC-A |
| chr1: 38136288 | ENST00000641212.1 | NA | Exon | SCLC-A |
| chr1: 39897235 | ENST00000397332.2 | MYCL | Exon | SCLC-A |
| chr1: 40314188 | ENST00000372736.3 | COL9A2 | Intron | SCLC-A |
| chr1: 40314213 | ENST00000372736.3 | COL9A2 | Intron | SCLC-A |
| chr1: 40314214 | ENST00000372736.3 | COL9A2 | Intron | SCLC-A |
| chr1: 40314222 | ENST00000372736.3 | COL9A2 | Intron | SCLC-A |
| chr1: 43997873 | ENST00000372307.7 | SLC6A9 | Intron | SCLC-A |
| chr1: 43997890 | ENST00000372307.7 | SLC6A9 | Intron | SCLC-A |
| chr1: 44289729 | ENST00000456170.6 | ERI3 | Intron | SCLC-A |
| chr1: 45520656 | ENST00000424390.1 | PRDX1 | Intron | SCLC-A |
| chr1: 45520711 | ENST00000424390.1 | PRDX1 | Intron | SCLC-A |
| chr1: 45520719 | ENST00000424390.1 | PRDX1 | Intron | SCLC-A |
| chr1: 50214781 | ENST00000440897.1 | NA | Intron | SCLC-A |
| chr1: 50214784 | ENST00000440897.1 | NA | Intron | SCLC-A |
| chr1: 50214848 | ENST00000440897.1 | NA | Intron | SCLC-A |
| chr1: 50214860 | ENST00000440897.1 | NA | Intron | SCLC-A |
| chr1: 50214888 | ENST00000440897.1 | NA | Intron | SCLC-A |
| chr1: 55050406 | ENST00000302118.5 | PCSK9 | Intron | SCLC-A |
| chr1: 55063357 | ENST00000302118.5 | PCSK9 | Intron | SCLC-A |
| chr1: 55063375 | ENST00000302118.5 | PCSK9 | Exon | SCLC-A |
| chr1: 55063383 | ENST00000302118.5 | PCSK9 | Exon | SCLC-A |
| chr1: 58922511 | ENST00000665413.1 | NA | Intron | SCLC-A |
| chr1: 58922515 | ENST00000665413.1 | NA | Intron | SCLC-A |
| chr1: 58922539 | ENST00000665413.1 | NA | Intron | SCLC-A |
| chr1: 58922540 | ENST00000665413.1 | NA | Intron | SCLC-A |
| chr1: 58922542 | ENST00000665413.1 | NA | Intron | SCLC-A |
| chr1: 58922543 | ENST00000665413.1 | NA | Intron | SCLC-A |
| chr1: 58922548 | ENST00000665413.1 | NA | Intron | SCLC-A |
| chr1: 58922549 | ENST00000665413.1 | NA | Intron | SCLC-A |
| chr1: 58922557 | ENST00000665413.1 | NA | Intron | SCLC-A |
| chr1: 60865294 | ENST00000665290.1 | LOC101926964 | Intron | SCLC-A |
| chr1: 60897152 | ENST00000371191.5 | NFIA | Intron | SCLC-A |
| chr1: 60897198 | ENST00000371191.5 | NFIA | Intron | SCLC-A |
| chr1: 60903747 | ENST00000371191.5 | NFIA | Intron | SCLC-A |
| chr1: 60903756 | ENST00000371191.5 | NFIA | Intron | SCLC-A |
| chr1: 60903781 | ENST00000371191.5 | NFIA | Intron | SCLC-A |
| chr1: 60903785 | ENST00000371191.5 | NFIA | Intron | SCLC-A |
| chr1: 60903893 | ENST00000371191.5 | NFIA | Intron | SCLC-A |
| chr1: 65173967 | ENST00000546702.5 | AK4 | Intron | SCLC-A |
| chr1: 110069330 | ENST00000473429.5 | STRIP1 | Intron | SCLC-A |
| chr1: 110069428 | ENST00000473429.5 | STRIP1 | Intron | SCLC-A |
| chr1: 110069452 | ENST00000473429.5 | STRIP1 | Intron | SCLC-A |
| chr1: 110069456 | ENST00000473429.5 | STRIP1 | Intron | SCLC-A |
| chr1: 110102148 | ENST00000554808.1 | LINC01397 | Intron | SCLC-A |
| chr1: 110102161 | ENST00000554808.1 | LINC01397 | Intron | SCLC-A |
| chr1: 110102162 | ENST00000554808.1 | LINC01397 | Intron | SCLC-A |
| chr1: 110102262 | ENST00000554808.1 | LINC01397 | Intron | SCLC-A |
| chr1: 112826812 | ENST00000433505.5 | LINC01356 | Intron | SCLC-A |
| chr1: 147545284 | ENST00000234739.8 | BCL9 | Intron | SCLC-A |
| chr1: 147545320 | ENST00000234739.8 | BCL9 | Intron | SCLC-A |
| chr1: 161909783 | ENST00000367942.4 | ATF6 | Intron | SCLC-A |
| chr1: 161909784 | ENST00000367942.4 | ATF6 | Intron | SCLC-A |
| chr1: 161909813 | ENST00000367942.4 | ATF6 | Intron | SCLC-A |
| chr1: 162373967 | ENST00000431696.1 | NOS1AP | Intron | SCLC-A |
| chr1: 162374123 | ENST00000431696.1 | NOS1AP | Intron | SCLC-A |
| chr1: 164638655 | ENST00000340699.7 | PBX1 | Intron | SCLC-A |
| chr1: 164638678 | ENST00000340699.7 | PBX1 | Intron | SCLC-A |
| chr1: 164638679 | ENST00000340699.7 | PBX1 | Intron | SCLC-A |
| chr1: 164638728 | ENST00000340699.7 | PBX1 | Intron | SCLC-A |
| chr1: 164638729 | ENST00000340699.7 | PBX1 | Intron | SCLC-A |
| chr1: 164638734 | ENST00000340699.7 | PBX1 | Intron | SCLC-A |
| chr1: 164638735 | ENST00000340699.7 | PBX1 | Intron | SCLC-A |
| chr1: 164638825 | ENST00000340699.7 | PBX1 | Intron | SCLC-A |
| chr1: 164797338 | ENST00000558796.2 | NA | Intron | SCLC-A |
| chr1: 180173996 | ENST00000367602.8 | QSOX1 | Intron | SCLC-A |
| chr1: 180928484 | ENST00000367587.1 | KIAA1614 | Exon | SCLC-A |
| chr1: 180928548 | ENST00000367587.1 | KIAA1614 | Exon | SCLC-A |
| chr1: 182236168 | ENST00000449842.2 | LINC01344 | Intron | SCLC-A |
| chr1: 182236190 | ENST00000449842.2 | LINC01344 | Intron | SCLC-A |
| chr1: 202199385 | ENST00000367278.7 | LGR6 | Intron | SCLC-A |
| chr1: 202637540 | ENST00000367268.5 | SYT2 | Intron | SCLC-A |
| chr1: 202637566 | ENST00000367268.5 | SYT2 | Intron | SCLC-A |
| chr1: 202637656 | ENST00000367268.5 | SYT2 | Intron | SCLC-A |
| chr1: 202637657 | ENST00000367268.5 | SYT2 | Intron | SCLC-A |
| chr1: 203506933 | ENST00000367222.7 | OPTC | Intron | SCLC-A |
| chr1: 204104288 | ENST00000530882.1 | SOX13 | Intron | SCLC-A |
| chr1: 204447561 | ENST00000367187.7 | PIK3C2B | Exon | SCLC-A |
| chr1: 204447662 | ENST00000367187.7 | PIK3C2B | Intron | SCLC-A |
| chr1: 204684469 | ENST00000367177.4 | LRRN2 | Intron | SCLC-A |
| chr1: 204684497 | ENST00000367177.4 | LRRN2 | Intron | SCLC-A |
| chr1: 204684501 | ENST00000367177.4 | LRRN2 | Intron | SCLC-A |
| chr1: 204684534 | ENST00000367177.4 | LRRN2 | Intron | SCLC-A |
| chr1: 204684542 | ENST00000367177.4 | LRRN2 | Intron | SCLC-A |
| chr1: 204684566 | ENST00000367177.4 | LRRN2 | Intron | SCLC-A |
| chr1: 204684591 | ENST00000367177.4 | LRRN2 | Intron | SCLC-A |
| chr1: 204684592 | ENST00000367177.4 | LRRN2 | Intron | SCLC-A |
| chr1: 204684660 | ENST00000367177.4 | LRRN2 | Intron | SCLC-A |
| chr1: 204684666 | ENST00000367177.4 | LRRN2 | Intron | SCLC-A |
| chr1: 204968154 | ENST00000339876.10 | NFASC | Intron | SCLC-A |
| chr1: 204968321 | ENST00000504149.1 | NFASC | Exon | SCLC-A |
| chr1: 205240223 | ENST00000358024.8 | TMCC2 | Intron | SCLC-A |
| chr1: 206490686 | ENST00000581977.7 | IKBKE | Intron | SCLC-A |
| chr1: 206490693 | ENST00000581977.7 | IKBKE | Intron | SCLC-A |
| chr1: 206490876 | ENST00000581977.7 | IKBKE | Intron | SCLC-A |
| chr1: 206490877 | ENST00000581977.7 | IKBKE | Intron | SCLC-A |
| chr1: 206490920 | ENST00000581977.7 | IKBKE | Intron | SCLC-A |
| chr1: 206490962 | ENST00000581977.7 | IKBKE | Intron | SCLC-A |
| chr1: 207873634 | ENST00000637970.1 | MIR29B2CHG | Intron | SCLC-A |
| chr1: 212979917 | ENST00000366966.6 | VASH2 | Intron | SCLC-A |
| chr1: 212980023 | ENST00000366966.6 | VASH2 | Intron | SCLC-A |
| chr1: 214551061 | ENST00000366956.10 | PTPN14 | Intron | SCLC-A |
| chr1: 214551098 | ENST00000366956.10 | PTPN14 | Intron | SCLC-A |
| chr1: 214551122 | ENST00000366956.10 | PTPN14 | Intron | SCLC-A |
| chr1: 226588954 | ENST00000366788.8 | STUM | Intron | SCLC-A |
| chr1: 226638084 | ENST00000272117.7 | ITPKB | Intron | SCLC-A |
| chr1: 226653615 | ENST00000272117.7 | ITPKB | Intron | SCLC-A |
| chr1: 234235838 | ENST00000366618.8 | SLC35F3 | Intron | SCLC-A |
| chr1: 245609842 | ENST00000407071.7 | KIF26B | Intron | SCLC-A |
| chr1: 245609882 | ENST00000407071.7 | KIF26B | Intron | SCLC-A |
| chr1: 245609932 | ENST00000407071.7 | KIF26B | Intron | SCLC-A |
| chr1: 246101776 | ENST00000630181.2 | SMYD3 | Intron | SCLC-A |
| chr10: 1650783 | ENST00000381312.6 | ADARB2 | Intron | SCLC-A |
| chr10: 1650884 | ENST00000381312.6 | ADARB2 | Intron | SCLC-A |
| chr10: 1652303 | ENST00000381312.6 | ADARB2 | Intron | SCLC-A |
| chr10: 1652318 | ENST00000381312.6 | ADARB2 | Intron | SCLC-A |
| chr10: 1652368 | ENST00000381312.6 | ADARB2 | Intron | SCLC-A |
| chr10: 1652396 | ENST00000381312.6 | ADARB2 | Intron | SCLC-A |
| chr10: 3045918 | ENST00000658425.1 | NA | Intron | SCLC-A |
| chr10: 3045932 | ENST00000658425.1 | NA | Intron | SCLC-A |
| chr10: 14605132 | ENST00000181796.6 | FAM107B | Intron | SCLC-A |
| chr10: 14605133 | ENST00000181796.6 | FAM107B | Intron | SCLC-A |
| chr10: 14605156 | ENST00000181796.6 | FAM107B | Intron | SCLC-A |
| chr10: 16684672 | ENST00000377921.7 | RSU1 | Intron | SCLC-A |
| chr10: 23960207 | ENST00000376462.5 | KIAA1217 | Intron | SCLC-A |
| chr10: 24187920 | ENST00000376462.5 | KIAA1217 | Intron | SCLC-A |
| chr10: 24278372 | ENST00000376462.5 | KIAA1217 | Intron | SCLC-A |
| chr10: 24293969 | ENST00000376462.5 | KIAA1217 | Intron | SCLC-A |
| chr10: 24294010 | ENST00000376462.5 | KIAA1217 | Intron | SCLC-A |
| chr10: 34795148 | ENST00000545260.5 | PARD3 | Intron | SCLC-A |
| chr10: 43082844 | ENST00000498820.5 | RET | Intron | SCLC-A |
| chr10: 51123452 | ENST00000401604.8 | PRKG1 | Intron | SCLC-A |
| chr10: 51123579 | ENST00000401604.8 | PRKG1 | Intron | SCLC-A |
| chr10: 71225616 | ENST00000335350.10 | UNC5B | Intron | SCLC-A |
| chr10: 71225620 | ENST00000335350.10 | UNC5B | Intron | SCLC-A |
| chr10: 71279282 | ENST00000335350.10 | UNC5B | Intron | SCLC-A |
| chr10: 71279303 | ENST00000335350.10 | UNC5B | Intron | SCLC-A |
| chr10: 71279304 | ENST00000335350.10 | UNC5B | Intron | SCLC-A |
| chr10: 71279323 | ENST00000335350.10 | UNC5B | Intron | SCLC-A |
| chr10: 71279324 | ENST00000335350.10 | UNC5B | Intron | SCLC-A |
| chr10: 71279401 | ENST00000335350.10 | UNC5B | Intron | SCLC-A |
| chr10: 71279426 | ENST00000335350.10 | UNC5B | Intron | SCLC-A |
| chr10: 71279427 | ENST00000335350.10 | UNC5B | Intron | SCLC-A |
| chr10: 71279454 | ENST00000335350.10 | UNC5B | Intron | SCLC-A |
| chr10: 71965349 | ENST00000373115.5 | CHST3 | Intron | SCLC-A |
| chr10: 71965350 | ENST00000373115.5 | CHST3 | Intron | SCLC-A |
| chr10: 71965369 | ENST00000373115.5 | CHST3 | Intron | SCLC-A |
| chr10: 71965370 | ENST00000373115.5 | CHST3 | Intron | SCLC-A |
| chr10: 71965378 | ENST00000373115.5 | CHST3 | Intron | SCLC-A |
| chr10: 71965400 | ENST00000373115.5 | CHST3 | Intron | SCLC-A |
| chr10: 71965440 | ENST00000373115.5 | CHST3 | Intron | SCLC-A |
| chr10: 71965474 | ENST00000373115.5 | CHST3 | Intron | SCLC-A |
| chr10: 74133949 | ENST00000355264.8 | AP3M1 | Intron | SCLC-A |
| chr10: 74134045 | ENST00000355264.8 | AP3M1 | Exon | SCLC-A |
| chr10: 74134046 | ENST00000355264.8 | AP3M1 | Exon | SCLC-A |
| chr10: 74134105 | ENST00000355264.8 | AP3M1 | Exon | SCLC-A |
| chr10: 74134108 | ENST00000355264.8 | AP3M1 | Exon | SCLC-A |
| chr10: 78963893 | ENST00000456353.5 | ZMIZ1-AS1 | Intron | SCLC-A |
| chr10: 78963937 | ENST00000456353.5 | ZMIZ1-AS1 | Intron | SCLC-A |
| chr10: 86672720 | ENST00000429277.6 | LDB3 | Intron | SCLC-A |
| chr10: 95355950 | ENST00000371227.8 | SORBS1 | Intron | SCLC-A |
| chr10: 95356062 | ENST00000371227.8 | SORBS1 | Intron | SCLC-A |
| chr10: 99167103 | ENST00000628193.2 | HPSE2 | Intron | SCLC-A |
| chr10: 99879859 | ENST00000543621.6 | DNMBP | Exon | SCLC-A |
| chr10: 101242381 | ENST00000434878.1 | NA | Intron | SCLC-A |
| chr10: 102294542 | ENST00000369983.3 | GBF1 | Intron | SCLC-A |
| chr10: 102294602 | ENST00000369983.3 | GBF1 | Intron | SCLC-A |
| chr10: 112952124 | ENST00000534894.5 | TCF7L2 | Intron | SCLC-A |
| chr10: 112952316 | ENST00000534894.5 | TCF7L2 | Intron | SCLC-A |
| chr10: 112952366 | ENST00000534894.5 | TCF7L2 | Intron | SCLC-A |
| chr10: 112952389 | ENST00000534894.5 | TCF7L2 | Intron | SCLC-A |
| chr10: 112952520 | ENST00000534894.5 | TCF7L2 | Intron | SCLC-A |
| chr10: 116275072 | ENST00000648277.1 | NA | Exon | SCLC-A |
| chr10: 116275113 | ENST00000648277.1 | NA | Exon | SCLC-A |
| chr10: 118809054 | ENST00000663084.1 | NA | Intron | SCLC-A |
| chr10: 118809074 | ENST00000663084.1 | NA | Intron | SCLC-A |
| chr10: 119282663 | ENST00000392870.3 | GRK5 | Intron | SCLC-A |
| chr10: 121580580 | ENST00000356226.8 | FGFR2 | Intron | SCLC-A |
| chr10: 121580749 | ENST00000356226.8 | FGFR2 | Intron | SCLC-A |
| chr10: 124482462 | ENST00000368842.10 | LHPP | Intron | SCLC-A |
| chr10: 124482496 | ENST00000368842.10 | LHPP | Intron | SCLC-A |
| chr10: 124482554 | ENST00000368842.10 | LHPP | Intron | SCLC-A |
| chr10: 127424585 | ENST00000280333.9 | DOCK1 | Intron | SCLC-A |
| chr10: 127424596 | ENST00000280333.9 | DOCK1 | Intron | SCLC-A |
| chr10: 127424597 | ENST00000280333.9 | DOCK1 | Intron | SCLC-A |
| chr10: 127424611 | ENST00000280333.9 | DOCK1 | Intron | SCLC-A |
| chr10: 127424612 | ENST00000280333.9 | DOCK1 | Intron | SCLC-A |
| chr10: 127424671 | ENST00000280333.9 | DOCK1 | Intron | SCLC-A |
| chr10: 127424672 | ENST00000280333.9 | DOCK1 | Intron | SCLC-A |
| chr10: 127424673 | ENST00000280333.9 | DOCK1 | Intron | SCLC-A |
| chr10: 127424674 | ENST00000280333.9 | DOCK1 | Intron | SCLC-A |
| chr10: 127424684 | ENST00000280333.9 | DOCK1 | Intron | SCLC-A |
| chr10: 129849527 | ENST00000368648.7 | EBF3 | Intron | SCLC-A |
| chr10: 129849536 | ENST00000368648.7 | EBF3 | Intron | SCLC-A |
| chr10: 129849548 | ENST00000368648.7 | EBF3 | Intron | SCLC-A |
| chr10: 129849583 | ENST00000368648.7 | EBF3 | Intron | SCLC-A |
| chr10: 129849589 | ENST00000368648.7 | EBF3 | Intron | SCLC-A |
| chr10: 129849601 | ENST00000368648.7 | EBF3 | Intron | SCLC-A |
| chr10: 129849604 | ENST00000368648.7 | EBF3 | Intron | SCLC-A |
| chr10: 129849607 | ENST00000368648.7 | EBF3 | Intron | SCLC-A |
| chr10: 132972142 | ENST00000461291.2 | LINC01168 | Exon | SCLC-A |
| chr10: 132972143 | ENST00000461291.2 | LINC01168 | Exon | SCLC-A |
| chr10: 133310352 | ENST00000482278.5 | TUBGCP2 | Intron | SCLC-A |
| chr10: 133310353 | ENST00000482278.5 | TUBGCP2 | Intron | SCLC-A |
| chr11: 888570 | ENST00000526714.5 | CHID1 | Intron | SCLC-A |
| chr11: 888640 | ENST00000526714.5 | CHID1 | Intron | SCLC-A |
| chr11: 1455421 | ENST00000308219.13 | BRSK2 | Intron | SCLC-A |
| chr11: 2718002 | ENST00000155840.11 | KCNQ1 | Intron | SCLC-A |
| chr11: 8775439 | ENST00000526057.5 | DENND2B | Intron | SCLC-A |
| chr11: 17449180 | ENST00000645744.1 | ABCC8 | Intron | SCLC-A |
| chr11: 19692207 | ENST00000360655.8 | NAV2 | Intron | SCLC-A |
| chr11: 19692236 | ENST00000360655.8 | NAV2 | Intron | SCLC-A |
| chr11: 19832407 | ENST00000360655.8 | NAV2 | Intron | SCLC-A |
| chr11: 19832412 | ENST00000360655.8 | NAV2 | Intron | SCLC-A |
| chr11: 19832444 | ENST00000360655.8 | NAV2 | Intron | SCLC-A |
| chr11: 19832445 | ENST00000360655.8 | NAV2 | Intron | SCLC-A |
| chr11: 19832522 | ENST00000360655.8 | NAV2 | Exon | SCLC-A |
| chr11: 19832523 | ENST00000360655.8 | NAV2 | Exon | SCLC-A |
| chr11: 19832532 | ENST00000360655.8 | NAV2 | Exon | SCLC-A |
| chr11: 19832570 | ENST00000360655.8 | NAV2 | Exon | SCLC-A |
| chr11: 19832571 | ENST00000360655.8 | NAV2 | Exon | SCLC-A |
| chr11: 19832660 | ENST00000360655.8 | NAV2 | Intron | SCLC-A |
| chr11: 22338216 | ENST00000263160.4 | SLC17A6 | Promoter | SCLC-A |
| chr11: 22338217 | ENST00000263160.4 | SLC17A6 | Promoter | SCLC-A |
| chr11: 22338234 | ENST00000263160.4 | SLC17A6 | Promoter | SCLC-A |
| chr11: 22338247 | ENST00000263160.4 | SLC17A6 | Promoter | SCLC-A |
| chr11: 22338248 | ENST00000263160.4 | SLC17A6 | Promoter | SCLC-A |
| chr11: 32092239 | ENST00000530348.5 | RCN1 | Intron | SCLC-A |
| chr11: 32092256 | ENST00000530348.5 | RCN1 | Intron | SCLC-A |
| chr11: 32092264 | ENST00000530348.5 | RCN1 | Intron | SCLC-A |
| chr11: 32092281 | ENST00000530348.5 | RCN1 | Intron | SCLC-A |
| chr11: 32434969 | ENST00000640146.2 | WT1 | Promoter | SCLC-A |
| chr11: 32435054 | ENST00000640146.2 | WT1 | Promoter | SCLC-A |
| chr11: 32435081 | ENST00000640146.2 | WT1 | Promoter | SCLC-A |
| chr11: 33645875 | ENST00000658780.1 | KIAA1549L | Exon | SCLC-A |
| chr11: 33645888 | ENST00000658780.1 | KIAA1549L | Exon | SCLC-A |
| chr11: 33645905 | ENST00000658780.1 | KIAA1549L | Exon | SCLC-A |
| chr11: 33890508 | ENST00000257818.3 | LMO2 | Intron | SCLC-A |
| chr11: 33890510 | ENST00000257818.3 | LMO2 | Intron | SCLC-A |
| chr11: 34345926 | ENST00000435224.3 | ABTB2 | Intron | SCLC-A |
| chr11: 34345939 | ENST00000435224.3 | ABTB2 | Intron | SCLC-A |
| chr11: 34502665 | ENST00000429939.6 | ELF5 | Intron | SCLC-A |
| chr11: 44593266 | ENST00000529277.5 | CD82 | Intron | SCLC-A |
| chr11: 44593342 | ENST00000529277.5 | CD82 | Intron | SCLC-A |
| chr11: 44593415 | ENST00000529277.5 | CD82 | Intron | SCLC-A |
| chr11: 44593416 | ENST00000529277.5 | CD82 | Intron | SCLC-A |
| chr11: 44593418 | ENST00000529277.5 | CD82 | Intron | SCLC-A |
| chr11: 44593458 | ENST00000529277.5 | CD82 | Intron | SCLC-A |
| chr11: 46273915 | ENST00000527239.1 | NA | Intron | SCLC-A |
| chr11: 46273916 | ENST00000527239.1 | NA | Intron | SCLC-A |
| chr11: 46894657 | ENST00000378623.6 | LRP4 | Exon | SCLC-A |
| chr11: 46894691 | ENST00000378623.6 | LRP4 | Exon | SCLC-A |
| chr11: 46894693 | ENST00000378623.6 | LRP4 | Exon | SCLC-A |
| chr11: 46894696 | ENST00000378623.6 | LRP4 | Exon | SCLC-A |
| chr11: 46894760 | ENST00000378623.6 | LRP4 | Exon | SCLC-A |
| chr11: 46918851 | ENST00000378623.6 | LRP4 | Promoter | SCLC-A |
| chr11: 46918857 | ENST00000378623.6 | LRP4 | Promoter | SCLC-A |
| chr11: 46918928 | ENST00000378623.6 | LRP4 | Promoter | SCLC-A |
| chr11: 60375860 | ENST00000532756.1 | MS4A6E | Intron | SCLC-A |
| chr11: 64146424 | ENST00000545464.5 | MACROD1 | Intron | SCLC-A |
| chr11: 64146425 | ENST00000545464.5 | MACROD1 | Intron | SCLC-A |
| chr11: 64146430 | ENST00000545464.5 | MACROD1 | Intron | SCLC-A |
| chr11: 64146431 | ENST00000545464.5 | MACROD1 | Intron | SCLC-A |
| chr11: 64146454 | ENST00000545464.5 | MACROD1 | Intron | SCLC-A |
| chr11: 64146460 | ENST00000545464.5 | MACROD1 | Intron | SCLC-A |
| chr11: 65552712 | ENST00000528966.5 | LTBP3 | Promoter | SCLC-A |
| chr11: 65552874 | ENST00000529764.1 | LTBP3 | Promoter | SCLC-A |
| chr11: 65552875 | ENST00000529764.1 | LTBP3 | Promoter | SCLC-A |
| chr11: 68010316 | ENST00000614849.4 | ALDH3B1 | Intron | SCLC-A |
| chr11: 68828883 | ENST00000561996.1 | CPT1A | Intron | SCLC-A |
| chr11: 68829295 | ENST00000561996.1 | CPT1A | Intron | SCLC-A |
| chr11: 70828728 | ENST00000601538.5 | SHANK2 | Intron | SCLC-A |
| chr11: 70828944 | ENST00000601538.5 | SHANK2 | Intron | SCLC-A |
| chr11: 71153151 | ENST00000601538.5 | SHANK2 | Intron | SCLC-A |
| chr11: 71153180 | ENST00000601538.5 | SHANK2 | Intron | SCLC-A |
| chr11: 75339869 | ENST00000420843.7 | ARRB1 | Intron | SCLC-A |
| chr11: 75339900 | ENST00000420843.7 | ARRB1 | Intron | SCLC-A |
| chr11: 75339977 | ENST00000420843.7 | ARRB1 | Intron | SCLC-A |
| chr11: 75339978 | ENST00000420843.7 | ARRB1 | Intron | SCLC-A |
| chr11: 107176910 | ENST00000526512.1 | NA | Exon | SCLC-A |
| chr11: 107176925 | ENST00000526512.1 | NA | Exon | SCLC-A |
| chr11: 119976142 | ENST00000664971.1 | NA | Exon | SCLC-A |
| chr11: 119976171 | ENST00000664971.1 | NA | Intron | SCLC-A |
| chr11: 119976172 | ENST00000664971.1 | NA | Intron | SCLC-A |
| chr11: 119976214 | ENST00000664971.1 | NA | Intron | SCLC-A |
| chr11: 121111282 | ENST00000645041.1 | TBCEL | Intron | SCLC-A |
| chr11: 126356252 | ENST00000629441.1 | GSEC | Promoter | SCLC-A |
| chr11: 126356902 | ENST00000227495.10 | ST3GAL4 | Intron | SCLC-A |
| chr11: 126585524 | ENST00000548204.1 | NA | Intron | SCLC-A |
| chr11: 126585526 | ENST00000548204.1 | NA | Intron | SCLC-A |
| chr11: 126852951 | ENST00000525144.7 | KIRREL3 | Intron | SCLC-A |
| chr11: 126852952 | ENST00000525144.7 | KIRREL3 | Intron | SCLC-A |
| chr11: 126852977 | ENST00000525144.7 | KIRREL3 | Intron | SCLC-A |
| chr11: 128720829 | ENST00000608303.5 | FLI1 | Intron | SCLC-A |
| chr11: 128726019 | ENST00000608303.5 | FLI1 | Intron | SCLC-A |
| chr12: 168200 | ENST00000538872.6 | IQSEC3 | Intron | SCLC-A |
| chr12: 543077 | ENST00000266383.10 | B4GALNT3 | Intron | SCLC-A |
| chr12: 1500545 | ENST00000515614.3 | LINC00942 | Exon | SCLC-A |
| chr12: 2846128 | ENST00000643039.1 | ITFG2 | Intron | SCLC-A |
| chr12: 2846129 | ENST00000643039.1 | ITFG2 | Intron | SCLC-A |
| chr12: 3077894 | ENST00000537971.5 | TSPAN9 | Intron | SCLC-A |
| chr12: 3077928 | ENST00000537971.5 | TSPAN9 | Intron | SCLC-A |
| chr12: 6340746 | ENST00000534885.5 | TNFRSF1A | Intron | SCLC-A |
| chr12: 6633709 | ENST00000329858.9 | LPAR5 | Intron | SCLC-A |
| chr12: 21829391 | ENST00000539874.1 | NA | Intron | SCLC-A |
| chr12: 21829497 | ENST00000539874.1 | NA | Intron | SCLC-A |
| chr12: 21841579 | ENST00000539874.1 | NA | Intron | SCLC-A |
| chr12: 21841649 | ENST00000539874.1 | NA | Intron | SCLC-A |
| chr12: 21868362 | ENST00000539874.1 | NA | Intron | SCLC-A |
| chr12: 30988328 | ENST00000546076.5 | TSPAN11 | Intron | SCLC-A |
| chr12: 42479753 | ENST00000639958.1 | PRICKLE1 | Intron | SCLC-A |
| chr12: 58742170 | ENST00000546977.5 | LOC100506869 | Intron | SCLC-A |
| chr12: 97465122 | ENST00000538559.6 | RMST | Intron | SCLC-A |
| chr12: 97465248 | ENST00000538559.6 | RMST | Intron | SCLC-A |
| chr12: 97465272 | ENST00000538559.6 | RMST | Intron | SCLC-A |
| chr12: 97465282 | ENST00000538559.6 | RMST | Intron | SCLC-A |
| chr12: 97465283 | ENST00000538559.6 | RMST | Intron | SCLC-A |
| chr12: 108446979 | ENST00000502160.6 | LINC01498 | Intron | SCLC-A |
| chr12: 109723834 | ENST00000538780.1 | FAM222A | Intron | SCLC-A |
| chr12: 119179398 | ENST00000541798.1 | HSPB8 | Promoter | SCLC-A |
| chr12: 119179459 | ENST00000541798.1 | HSPB8 | Promoter | SCLC-A |
| chr12: 119179460 | ENST00000541798.1 | HSPB8 | Promoter | SCLC-A |
| chr12: 119179509 | ENST00000541798.1 | HSPB8 | Promoter | SCLC-A |
| chr12: 121790098 | ENST00000267205.7 | RHOF | Intron | SCLC-A |
| chr12: 121793404 | ENST00000546227.5 | RHOF | Intron | SCLC-A |
| chr12: 124259751 | ENST00000389727.8 | ZNF664-RFLNA | Intron | SCLC-A |
| chr12: 124259755 | ENST00000389727.8 | ZNF664-RFLNA | Intron | SCLC-A |
| chr12: 124259756 | ENST00000389727.8 | ZNF664-RFLNA | Intron | SCLC-A |
| chr12: 124259764 | ENST00000389727.8 | ZNF664-RFLNA | Intron | SCLC-A |
| chr12: 124259765 | ENST00000389727.8 | ZNF664-RFLNA | Intron | SCLC-A |
| chr12: 124259769 | ENST00000389727.8 | ZNF664-RFLNA | Intron | SCLC-A |
| chr12: 124259805 | ENST00000389727.8 | ZNF664-RFLNA | Intron | SCLC-A |
| chr12: 124259869 | ENST00000389727.8 | ZNF664-RFLNA | Intron | SCLC-A |
| chr12: 124259875 | ENST00000389727.8 | ZNF664-RFLNA | Intron | SCLC-A |
| chr12: 124289987 | ENST00000389727.8 | ZNF664-RFLNA | Intron | SCLC-A |
| chr12: 124290024 | ENST00000389727.8 | ZNF664-RFLNA | Intron | SCLC-A |
| chr12: 124290027 | ENST00000389727.8 | ZNF664-RFLNA | Intron | SCLC-A |
| chr12: 130460794 | ENST00000261655.8 | RIMBP2 | Intron | SCLC-A |
| chr12: 130460795 | ENST00000261655.8 | RIMBP2 | Intron | SCLC-A |
| chr12: 130460796 | ENST00000261655.8 | RIMBP2 | Intron | SCLC-A |
| chr12: 130461043 | ENST00000261655.8 | RIMBP2 | Intron | SCLC-A |
| chr12: 130461044 | ENST00000261655.8 | RIMBP2 | Intron | SCLC-A |
| chr12: 130461173 | ENST00000261655.8 | RIMBP2 | Intron | SCLC-A |
| chr13: 25964027 | ENST00000381655.7 | ATP8A2 | Intron | SCLC-A |
| chr13: 25964055 | ENST00000381655.7 | ATP8A2 | Intron | SCLC-A |
| chr13: 27961367 | ENST00000381020.8 | CDX2 | Exon | SCLC-A |
| chr13: 29567470 | ENST00000380752.10 | SLC7A1 | Intron | SCLC-A |
| chr13: 29567546 | ENST00000380752.10 | SLC7A1 | Intron | SCLC-A |
| chr13: 39600949 | ENST00000648377.1 | LHFPL6 | Exon | SCLC-A |
| chr13: 39601041 | ENST00000648377.1 | LHFPL6 | Exon | SCLC-A |
| chr13: 39601042 | ENST00000648377.1 | LHFPL6 | Exon | SCLC-A |
| chr13: 39601075 | ENST00000648377.1 | LHFPL6 | Exon | SCLC-A |
| chr13: 50129918 | ENST00000476738.5 | DLEU1 | Intron | SCLC-A |
| chr13: 50129964 | ENST00000476738.5 | DLEU1 | Intron | SCLC-A |
| chr13: 50129976 | ENST00000476738.5 | DLEU1 | Intron | SCLC-A |
| chr13: 50130078 | ENST00000476738.5 | DLEU1 | Intron | SCLC-A |
| chr13: 94968694 | ENST00000669430.1 | NA | Intron | SCLC-A |
| chr13: 94968800 | ENST00000669430.1 | NA | Intron | SCLC-A |
| chr13: 94968807 | ENST00000669430.1 | NA | Intron | SCLC-A |
| chr13: 94968841 | ENST00000669430.1 | NA | Intron | SCLC-A |
| chr13: 94968846 | ENST00000669430.1 | NA | Intron | SCLC-A |
| chr13: 94968880 | ENST00000669430.1 | NA | Intron | SCLC-A |
| chr13: 94968943 | ENST00000669430.1 | NA | Intron | SCLC-A |
| chr13: 94968944 | ENST00000669430.1 | NA | Intron | SCLC-A |
| chr13: 94968987 | ENST00000669430.1 | NA | Intron | SCLC-A |
| chr13: 98214022 | ENST00000595437.5 | FARP1 | Intron | SCLC-A |
| chr13: 98214053 | ENST00000595437.5 | FARP1 | Intron | SCLC-A |
| chr13: 98214104 | ENST00000595437.5 | FARP1 | Intron | SCLC-A |
| chr13: 98214113 | ENST00000595437.5 | FARP1 | Intron | SCLC-A |
| chr13: 98214154 | ENST00000595437.5 | FARP1 | Intron | SCLC-A |
| chr13: 98214201 | ENST00000595437.5 | FARP1 | Intron | SCLC-A |
| chr13: 98214220 | ENST00000595437.5 | FARP1 | Intron | SCLC-A |
| chr13: 98214229 | ENST00000595437.5 | FARP1 | Intron | SCLC-A |
| chr13: 107868214 | ENST00000375915.4 | FAM155A | Promoter | SCLC-A |
| chr13: 111598391 | ENST00000375713.1 | NA | Intron | SCLC-A |
| chr13: 111598398 | ENST00000375713.1 | NA | Intron | SCLC-A |
| chr13: 114161911 | ENST00000643687.1 | NA | Intron | SCLC-A |
| chr13: 114161926 | ENST00000643687.1 | NA | Intron | SCLC-A |
| chr14: 70742303 | ENST00000611979.4 | MAP3K9 | Intron | SCLC-A |
| chr14: 74917011 | ENST00000555009.5 | RPS6KL1 | Intron | SCLC-A |
| chr14: 76261732 | ENST00000554225.1 | NA | Intron | SCLC-A |
| chr14: 76261754 | ENST00000554225.1 | NA | Intron | SCLC-A |
| chr14: 76396981 | ENST00000512784.6 | ESRRB | Intron | SCLC-A |
| chr14: 76397007 | ENST00000512784.6 | ESRRB | Intron | SCLC-A |
| chr14: 90600821 | ENST00000328459.11 | TTC7B | Intron | SCLC-A |
| chr14: 90700943 | ENST00000328459.11 | TTC7B | Intron | SCLC-A |
| chr14: 91895601 | ENST00000267620.14 | FBLN5 | Intron | SCLC-A |
| chr14: 91895602 | ENST00000267620.14 | FBLN5 | Intron | SCLC-A |
| chr14: 93737704 | ENST00000393143.5 | PRIMA1 | Intron | SCLC-A |
| chr14: 93737727 | ENST00000393143.5 | PRIMA1 | Intron | SCLC-A |
| chr14: 93962999 | ENST00000556337.1 | ASB2 | Intron | SCLC-A |
| chr14: 93963010 | ENST00000556337.1 | ASB2 | Intron | SCLC-A |
| chr14: 95259420 | ENST00000298912.9 | CLMN | Intron | SCLC-A |
| chr14: 95877410 | ENST00000504119.1 | TUNAR | Intron | SCLC-A |
| chr14: 99760240 | ENST00000554479.5 | EML1 | Intron | SCLC-A |
| chr14: 100903298 | ENST00000637474.1 | MEG8 | Intron | SCLC-A |
| chr14: 102001006 | ENST00000643684.1 | DYNC1H1 | Exon | SCLC-A |
| chr15: 28884288 | ENST00000669290.1 | NA | Intron | SCLC-A |
| chr15: 28884378 | ENST00000561069.5 | APBA2 | Promoter | SCLC-A |
| chr15: 34580533 | ENST00000569100.5 | GOLGA8B | Intron | SCLC-A |
| chr15: 60398693 | ENST00000557904.5 | ANXA2 | Intron | SCLC-A |
| chr15: 60398722 | ENST00000557904.5 | ANXA2 | Intron | SCLC-A |
| chr15: 60398724 | ENST00000557904.5 | ANXA2 | Intron | SCLC-A |
| chr15: 60398749 | ENST00000557904.5 | ANXA2 | Intron | SCLC-A |
| chr15: 65271083 | ENST00000444347.2 | PARP16 | Intron | SCLC-A |
| chr15: 74826644 | ENST00000564823.1 | CPLX3 | Exon | SCLC-A |
| chr15: 78649932 | ENST00000560511.5 | CHRNB4 | Intron | SCLC-A |
| chr15: 80559650 | ENST00000303329.9 | ARNT2 | Intron | SCLC-A |
| chr15: 81271331 | ENST00000560241.5 | IL16 | Intron | SCLC-A |
| chr15: 89647015 | ENST00000394412.8 | KIF7 | Exon | SCLC-A |
| chr15: 89815295 | ENST00000300060.7 | ANPEP | Promoter | SCLC-A |
| chr15: 90660767 | ENST00000559531.1 | CRTC3-AS1 | Intron | SCLC-A |
| chr15: 96321658 | ENST00000502125.6 | NR2F2-AS1 | Intron | SCLC-A |
| chr15: 98998036 | ENST00000559468.1 | NA | Intron | SCLC-A |
| chr15: 100111195 | ENST00000268070.9 | ADAMTS17 | Intron | SCLC-A |
| chr16: 71901 | ENST00000450643.5 | RHBDF1 | Intron | SCLC-A |
| chr16: 71908 | ENST00000450643.5 | RHBDF1 | Intron | SCLC-A |
| chr16: 301937 | ENST00000461023.5 | AXIN1 | Intron | SCLC-A |
| chr16: 301947 | ENST00000461023.5 | AXIN1 | Intron | SCLC-A |
| chr16: 528881 | ENST00000219611.7 | CAPN15 | Intron | SCLC-A |
| chr16: 528917 | ENST00000219611.7 | CAPN15 | Intron | SCLC-A |
| chr16: 528918 | ENST00000219611.7 | CAPN15 | Intron | SCLC-A |
| chr16: 528931 | ENST00000219611.7 | CAPN15 | Intron | SCLC-A |
| chr16: 528932 | ENST00000219611.7 | CAPN15 | Intron | SCLC-A |
| chr16: 629494 | ENST00000573440.1 | NA | Exon | SCLC-A |
| chr16: 629543 | ENST00000573440.1 | NA | Exon | SCLC-A |
| chr16: 629643 | ENST00000573440.1 | NA | Exon | SCLC-A |
| chr16: 629796 | ENST00000573440.1 | NA | Exon | SCLC-A |
| chr16: 629918 | ENST00000573440.1 | NA | Exon | SCLC-A |
| chr16: 1158479 | ENST00000638323.1 | CACNA1H | Intron | SCLC-A |
| chr16: 1158560 | ENST00000638323.1 | CACNA1H | Intron | SCLC-A |
| chr16: 1158565 | ENST00000638323.1 | CACNA1H | Intron | SCLC-A |
| chr16: 1158758 | ENST00000638323.1 | CACNA1H | Intron | SCLC-A |
| chr16: 1158983 | ENST00000638323.1 | CACNA1H | Intron | SCLC-A |
| chr16: 1159004 | ENST00000638323.1 | CACNA1H | Intron | SCLC-A |
| chr16: 1159245 | ENST00000638323.1 | CACNA1H | Intron | SCLC-A |
| chr16: 1159298 | ENST00000638323.1 | CACNA1H | Intron | SCLC-A |
| chr16: 2283053 | ENST00000569062.1 | ABCA3 | Intron | SCLC-A |
| chr16: 2283141 | ENST00000569062.1 | ABCA3 | Intron | SCLC-A |
| chr16: 2283155 | ENST00000569062.1 | ABCA3 | Intron | SCLC-A |
| chr16: 2283202 | ENST00000569062.1 | ABCA3 | Exon | SCLC-A |
| chr16: 4260046 | ENST00000204517.11 | TFAP4 | Intron | SCLC-A |
| chr16: 4260193 | ENST00000575320.1 | TFAP4 | Exon | SCLC-A |
| chr16: 4260231 | ENST00000575320.1 | TFAP4 | Exon | SCLC-A |
| chr16: 4260760 | ENST00000574639.1 | TFAP4 | Intron | SCLC-A |
| chr16: 4892618 | ENST00000592772.1 | PPL | Intron | SCLC-A |
| chr16: 6189354 | ENST00000569895.3 | RBFOX1 | Intron | SCLC-A |
| chr16: 6189356 | ENST00000569895.3 | RBFOX1 | Intron | SCLC-A |
| chr16: 8776423 | ENST00000268251.13 | ABAT | Exon | SCLC-A |
| chr16: 11349782 | ENST00000649869.1 | NA | Intron | SCLC-A |
| chr16: 11349931 | ENST00000649869.1 | NA | Intron | SCLC-A |
| chr16: 11387124 | ENST00000599216.5 | LOC400499 | Exon | SCLC-A |
| chr16: 11387125 | ENST00000599216.5 | LOC400499 | Exon | SCLC-A |
| chr16: 11387135 | ENST00000599216.5 | LOC400499 | Exon | SCLC-A |
| chr16: 11387138 | ENST00000599216.5 | LOC400499 | Exon | SCLC-A |
| chr16: 11411119 | ENST00000596416.1 | LOC400499 | Intron | SCLC-A |
| chr16: 11609545 | ENST00000574848.5 | LITAF | Intron | SCLC-A |
| chr16: 11609559 | ENST00000574848.5 | LITAF | Intron | SCLC-A |
| chr16: 11613760 | ENST00000574848.5 | LITAF | Intron | SCLC-A |
| chr16: 16067336 | ENST00000399408.6 | ABCC1 | Intron | SCLC-A |
| chr16: 16067355 | ENST00000399408.6 | ABCC1 | Intron | SCLC-A |
| chr16: 16067356 | ENST00000399408.6 | ABCC1 | Intron | SCLC-A |
| chr16: 16101762 | ENST00000399410.8 | ABCC1 | Intron | SCLC-A |
| chr16: 16101824 | ENST00000399410.8 | ABCC1 | Intron | SCLC-A |
| chr16: 21511849 | ENST00000522841.6 | SMG1P3 | Intron | SCLC-A |
| chr16: 21514060 | ENST00000522841.6 | SMG1P3 | Intron | SCLC-A |
| chr16: 21514076 | ENST00000522841.6 | SMG1P3 | Intron | SCLC-A |
| chr16: 21514077 | ENST00000522841.6 | SMG1P3 | Intron | SCLC-A |
| chr16: 21514208 | ENST00000522841.6 | SMG1P3 | Intron | SCLC-A |
| chr16: 53983658 | ENST00000636992.1 | FTO | Intron | SCLC-A |
| chr16: 54054484 | ENST00000471389.6 | FTO | Intron | SCLC-A |
| chr16: 54054552 | ENST00000471389.6 | FTO | Intron | SCLC-A |
| chr16: 54054572 | ENST00000471389.6 | FTO | Intron | SCLC-A |
| chr16: 56961212 | ENST00000379780.6 | CETP | Promoter | SCLC-A |
| chr16: 57115437 | ENST00000290776.13 | CPNE2 | Intron | SCLC-A |
| chr16: 57475091 | ENST00000566936.5 | DOK4 | Intron | SCLC-A |
| chr16: 57863783 | ENST00000563028.1 | KIFC3 | Promoter | SCLC-A |
| chr16: 57863784 | ENST00000563028.1 | KIFC3 | Promoter | SCLC-A |
| chr16: 58474357 | ENST00000568999.5 | NDRG4 | Intron | SCLC-A |
| chr16: 64947737 | ENST00000394156.7 | CDH11 | Exon | SCLC-A |
| chr16: 64997037 | ENST00000394156.7 | CDH11 | Intron | SCLC-A |
| chr16: 64997126 | ENST00000394156.7 | CDH11 | Intron | SCLC-A |
| chr16: 68590727 | ENST00000612545.1 | NA | Promoter | SCLC-A |
| chr16: 68590747 | ENST00000612545.1 | NA | Promoter | SCLC-A |
| chr16: 71627044 | ENST00000565261.1 | MARVELD3 | Intron | SCLC-A |
| chr16: 71627062 | ENST00000565261.1 | MARVELD3 | Intron | SCLC-A |
| chr16: 71627087 | ENST00000565261.1 | MARVELD3 | Intron | SCLC-A |
| chr16: 71627110 | ENST00000565261.1 | MARVELD3 | Intron | SCLC-A |
| chr16: 71627142 | ENST00000565261.1 | MARVELD3 | Intron | SCLC-A |
| chr16: 71627229 | ENST00000565261.1 | MARVELD3 | Intron | SCLC-A |
| chr16: 71627231 | ENST00000565261.1 | MARVELD3 | Intron | SCLC-A |
| chr16: 71627269 | ENST00000565261.1 | MARVELD3 | Intron | SCLC-A |
| chr16: 71627274 | ENST00000565261.1 | MARVELD3 | Intron | SCLC-A |
| chr16: 71627278 | ENST00000565261.1 | MARVELD3 | Intron | SCLC-A |
| chr16: 71627429 | ENST00000565261.1 | MARVELD3 | Intron | SCLC-A |
| chr16: 77789719 | ENST00000302536.3 | VAT1L | Intron | SCLC-A |
| chr16: 77789811 | ENST00000302536.3 | VAT1L | Intron | SCLC-A |
| chr16: 77789828 | ENST00000302536.3 | VAT1L | Intron | SCLC-A |
| chr16: 78580325 | ENST00000402655.6 | WWOX | Intron | SCLC-A |
| chr16: 78580326 | ENST00000402655.6 | WWOX | Intron | SCLC-A |
| chr16: 78597146 | ENST00000402655.6 | WWOx | Intron | SCLC-A |
| chr16: 78993665 | ENST00000402655.6 | WWOX | Intron | SCLC-A |
| chr16: 78993668 | ENST00000402655.6 | WWOX | Intron | SCLC-A |
| chr16: 78993669 | ENST00000402655.6 | WWOX | Intron | SCLC-A |
| chr16: 78993688 | ENST00000402655.6 | WWOx | Intron | SCLC-A |
| chr16: 78993753 | ENST00000402655.6 | WWOX | Intron | SCLC-A |
| chr16: 78993754 | ENST00000402655.6 | WWOX | Intron | SCLC-A |
| chr16: 78993777 | ENST00000402655.6 | WWOX | Intron | SCLC-A |
| chr16: 85171183 | ENST00000637419.1 | GSE1 | Exon | SCLC-A |
| chr16: 85171201 | ENST00000637419.1 | GSE1 | Exon | SCLC-A |
| chr16: 85171330 | ENST00000637419.1 | GSE1 | Exon | SCLC-A |
| chr16: 85214654 | ENST00000637419.1 | GSE1 | Intron | SCLC-A |
| chr16: 85247654 | ENST00000637419.1 | GSE1 | Intron | SCLC-A |
| chr16: 85287017 | ENST00000637419.1 | GSE1 | Intron | SCLC-A |
| chr16: 85287049 | ENST00000637419.1 | GSE1 | Intron | SCLC-A |
| chr16: 85287080 | ENST00000637419.1 | GSE1 | Intron | SCLC-A |
| chr16: 85287095 | ENST00000637419.1 | GSE1 | Intron | SCLC-A |
| chr16: 85287116 | ENST00000637419.1 | GSE1 | Intron | SCLC-A |
| chr16: 85287162 | ENST00000637419.1 | GSE1 | Intron | SCLC-A |
| chr16: 85354629 | ENST00000637419.1 | GSE1 | Intron | SCLC-A |
| chr16: 85379139 | ENST00000637419.1 | GSE1 | Intron | SCLC-A |
| chr16: 85381912 | ENST00000637419.1 | GSE1 | Intron | SCLC-A |
| chr16: 85382047 | ENST00000637419.1 | GSE1 | Intron | SCLC-A |
| chr16: 88886879 | ENST00000565053.1 | LOC101927793 | Exon | SCLC-A |
| chr16: 88886886 | ENST00000565053.1 | LOC101927793 | Exon | SCLC-A |
| chr16: 88886908 | ENST00000565053.1 | LOC101927793 | Exon | SCLC-A |
| chr16: 88886922 | ENST00000565053.1 | LOC101927793 | Exon | SCLC-A |
| chr16: 88886924 | ENST00000565053.1 | LOC101927793 | Exon | SCLC-A |
| chr16: 88886931 | ENST00000565053.1 | LOC101927793 | Exon | SCLC-A |
| chr16: 88886934 | ENST00000565053.1 | LOC101927793 | Exon | SCLC-A |
| chr16: 88886939 | ENST00000565053.1 | LOC101927793 | Exon | SCLC-A |
| chr16: 88886970 | ENST00000565053.1 | LOC101927793 | Exon | SCLC-A |
| chr16: 88886977 | ENST00000565053.1 | LOC101927793 | Exon | SCLC-A |
| chr16: 88887475 | ENST00000327483.9 | CBFA2T3 | Intron | SCLC-A |
| chr16: 88887480 | ENST00000327483.9 | CBFA2T3 | Intron | SCLC-A |
| chr16: 88887588 | ENST00000327483.9 | CBFA2T3 | Intron | SCLC-A |
| chr16: 88887597 | ENST00000327483.9 | CBFA2T3 | Intron | SCLC-A |
| chr16: 88922613 | ENST00000268679.9 | CBFA2T3 | Intron | SCLC-A |
| chr16: 88936828 | ENST00000268679.9 | CBFA2T3 | Intron | SCLC-A |
| chr17: 1998369 | ENST00000331238.7 | RTN4RL1 | Intron | SCLC-A |
| chr17: 1998382 | ENST00000331238.7 | RTN4RL1 | Intron | SCLC-A |
| chr17: 1998395 | ENST00000331238.7 | RTN4RL1 | Intron | SCLC-A |
| chr17: 1998403 | ENST00000331238.7 | RTN4RL1 | Intron | SCLC-A |
| chr17: 1998405 | ENST00000331238.7 | RTN4RL1 | Intron | SCLC-A |
| chr17: 1998409 | ENST00000331238.7 | RTN4RL1 | Intron | SCLC-A |
| chr17: 1998411 | ENST00000331238.7 | RTN4RL1 | Intron | SCLC-A |
| chr17: 1998431 | ENST00000331238.7 | RTN4RL1 | Intron | SCLC-A |
| chr17: 2174707 | ENST00000263073.11 | SMG6 | Intron | SCLC-A |
| chr17: 2174708 | ENST00000263073.11 | SMG6 | Intron | SCLC-A |
| chr17: 3911669 | ENST00000572418.1 | P2RX1 | Intron | SCLC-A |
| chr17: 3911674 | ENST00000572418.1 | P2RX1 | Intron | SCLC-A |
| chr17: 3911701 | ENST00000572418.1 | P2RX1 | Intron | SCLC-A |
| chr17: 3911707 | ENST00000572418.1 | P2RX1 | Intron | SCLC-A |
| chr17: 7384105 | ENST00000577009.1 | TNK1 | Promoter | SCLC-A |
| chr17: 7384131 | ENST00000577009.1 | TNK1 | Promoter | SCLC-A |
| chr17: 7384149 | ENST00000577009.1 | TNK1 | Promoter | SCLC-A |
| chr17: 7384152 | ENST00000577009.1 | TNK1 | Promoter | SCLC-A |
| chr17: 7384179 | ENST00000577009.1 | TNK1 | Promoter | SCLC-A |
| chr17: 7384180 | ENST00000577009.1 | TNK1 | Promoter | SCLC-A |
| chr17: 7384190 | ENST00000577009.1 | TNK1 | Promoter | SCLC-A |
| chr17: 9212784 | ENST00000173229.7 | NTN1 | Intron | SCLC-A |
| chr17: 9212826 | ENST00000173229.7 | NTN1 | Intron | SCLC-A |
| chr17: 9212846 | ENST00000173229.7 | NTN1 | Intron | SCLC-A |
| chr17: 9212875 | ENST00000173229.7 | NTN1 | Intron | SCLC-A |
| chr17: 9828889 | ENST00000574745.5 | GLP2R | Intron | SCLC-A |
| chr17: 9847489 | ENST00000574745.5 | GLP2R | Intron | SCLC-A |
| chr17: 10570616 | ENST00000587182.2 | MYHAS | Intron | SCLC-A |
| chr17: 10570716 | ENST00000587182.2 | MYHAS | Intron | SCLC-A |
| chr17: 10621565 | ENST00000587182.2 | MYHAS | Intron | SCLC-A |
| chr17: 11202000 | ENST00000648331.1 | NA | Intron | SCLC-A |
| chr17: 19003757 | ENST00000417251.6 | SLC5A10 | Intron | SCLC-A |
| chr17: 19003791 | ENST00000417251.6 | SLC5A10 | Intron | SCLC-A |
| chr17: 19003814 | ENST00000417251.6 | SLC5A10 | Intron | SCLC-A |
| chr17: 19003819 | ENST00000417251.6 | SLC5A10 | Intron | SCLC-A |
| chr17: 19003820 | ENST00000417251.6 | SLC5A10 | Intron | SCLC-A |
| chr17: 19003828 | ENST00000417251.6 | SLC5A10 | Intron | SCLC-A |
| chr17: 19003829 | ENST00000417251.6 | SLC5A10 | Intron | SCLC-A |
| chr17: 19003845 | ENST00000417251.6 | SLC5A10 | Intron | SCLC-A |
| chr17: 19003846 | ENST00000417251.6 | SLC5A10 | Intron | SCLC-A |
| chr17: 19003862 | ENST00000417251.6 | SLC5A10 | Intron | SCLC-A |
| chr17: 19003875 | ENST00000417251.6 | SLC5A10 | Intron | SCLC-A |
| chr17: 19003887 | ENST00000417251.6 | SLC5A10 | Intron | SCLC-A |
| chr17: 19003888 | ENST00000417251.6 | SLC5A10 | Intron | SCLC-A |
| chr17: 19003909 | ENST00000417251.6 | SLC5A10 | Intron | SCLC-A |
| chr17: 20256098 | ENST00000580225.1 | NA | Intron | SCLC-A |
| chr17: 20256112 | ENST00000580225.1 | NA | Intron | SCLC-A |
| chr17: 21453265 | ENST00000654263.1 | NA | Intron | SCLC-A |
| chr17: 21453285 | ENST00000654263.1 | NA | Intron | SCLC-A |
| chr17: 31409827 | ENST00000621161.5 | RAB11FIP4 | Intron | SCLC-A |
| chr17: 37507513 | ENST00000617516.5 | DUSP14 | Intron | SCLC-A |
| chr17: 37512705 | ENST00000614411.1 | DUSP14 | Exon | SCLC-A |
| chr17: 37512706 | ENST00000614411.1 | DUSP14 | Exon | SCLC-A |
| chr17: 37512726 | ENST00000614411.1 | DUSP14 | Exon | SCLC-A |
| chr17: 37512885 | ENST00000614411.1 | DUSP14 | Exon | SCLC-A |
| chr17: 37512896 | ENST00000614411.1 | DUSP14 | Exon | SCLC-A |
| chr17: 37512897 | ENST00000614411.1 | DUSP14 | Exon | SCLC-A |
| chr17: 39700220 | ENST00000578199.5 | ERBB2 | Intron | SCLC-A |
| chr17: 39700343 | ENST00000578199.5 | ERBB2 | Intron | SCLC-A |
| chr17: 39700370 | ENST00000578199.5 | ERBB2 | Intron | SCLC-A |
| chr17: 39700381 | ENST00000578199.5 | ERBB2 | Intron | SCLC-A |
| chr17: 39700383 | ENST00000578199.5 | ERBB2 | Intron | SCLC-A |
| chr17: 39700475 | ENST00000578199.5 | ERBB2 | Intron | SCLC-A |
| chr17: 39700479 | ENST00000578199.5 | ERBB2 | Intron | SCLC-A |
| chr17: 39700503 | ENST00000578199.5 | ERBB2 | Intron | SCLC-A |
| chr17: 39700508 | ENST00000578199.5 | ERBB2 | Intron | SCLC-A |
| chr17: 45688870 | ENST00000634540.1 | LINC02210-CRHR1 | Intron | SCLC-A |
| chr17: 45688887 | ENST00000634540.1 | LINC02210-CRHR1 | Intron | SCLC-A |
| chr17: 48543085 | ENST00000504972.3 | HOXB-AS1 | Promoter | SCLC-A |
| chr17: 48543181 | ENST00000504972.3 | HOXB-AS1 | Promoter | SCLC-A |
| chr17: 48543202 | ENST00000504972.3 | HOXB-AS1 | Promoter | SCLC-A |
| chr17: 48544564 | ENST00000504972.3 | HOXB-AS1 | Intron | SCLC-A |
| chr17: 48550531 | ENST00000470495.1 | HOXB3 | Exon | SCLC-A |
| chr17: 48550541 | ENST00000470495.1 | HOXB3 | Exon | SCLC-A |
| chr17: 48550542 | ENST00000470495.1 | HOXB3 | Exon | SCLC-A |
| chr17: 48550547 | ENST00000470495.1 | HOXB3 | Exon | SCLC-A |
| chr17: 48550548 | ENST00000470495.1 | HOXB3 | Exon | SCLC-A |
| chr17: 48550553 | ENST00000470495.1 | HOXB3 | Exon | SCLC-A |
| chr17: 48550554 | ENST00000470495.1 | HOXB3 | Exon | SCLC-A |
| chr17: 48550555 | ENST00000470495.1 | HOXB3 | Exon | SCLC-A |
| chr17: 48550556 | ENST00000470495.1 | HOXB3 | Exon | SCLC-A |
| chr17: 48550561 | ENST00000470495.1 | HOXB3 | Exon | SCLC-A |
| chr17: 48550568 | ENST00000470495.1 | HOXB3 | Exon | SCLC-A |
| chr17: 48550573 | ENST00000470495.1 | HOXB3 | Exon | SCLC-A |
| chr17: 48550581 | ENST00000470495.1 | HOXB3 | Exon | SCLC-A |
| chr17: 48550638 | ENST00000470495.1 | HOXB3 | Exon | SCLC-A |
| chr17: 48550914 | ENST00000470495.1 | HOXB3 | Exon | SCLC-A |
| chr17: 48550941 | ENST00000470495.1 | HOXB3 | Exon | SCLC-A |
| chr17: 48550945 | ENST00000470495.1 | HOXB3 | Exon | SCLC-A |
| chr17: 48551664 | ENST00000465846.6 | HOXB-AS3 | Intron | SCLC-A |
| chr17: 48578115 | ENST00000465846.6 | HOXB-AS3 | Intron | SCLC-A |
| chr17: 48578123 | ENST00000465846.6 | HOXB-AS3 | Intron | SCLC-A |
| chr17: 48578125 | ENST00000465846.6 | HOXB-AS3 | Intron | SCLC-A |
| chr17: 48578130 | ENST00000465846.6 | HOXB-AS3 | Intron | SCLC-A |
| chr17: 48578137 | ENST00000465846.6 | HOXB-AS3 | Intron | SCLC-A |
| chr17: 48578165 | ENST00000465846.6 | HOXB-AS3 | Intron | SCLC-A |
| chr17: 48578171 | ENST00000465846.6 | HOXB-AS3 | Intron | SCLC-A |
| chr17: 50170246 | ENST00000513942.5 | SGCA | Intron | SCLC-A |
| chr17: 50194705 | ENST00000476387.1 | COL1A1 | Promoter | SCLC-A |
| chr17: 50194723 | ENST00000476387.1 | COL1A1 | Promoter | SCLC-A |
| chr17: 50194727 | ENST00000476387.1 | COL1A1 | Promoter | SCLC-A |
| chr17: 50194739 | ENST00000476387.1 | COL1A1 | Promoter | SCLC-A |
| chr17: 50469399 | ENST00000510410.5 | ACSF2 | Intron | SCLC-A |
| chr17: 57291116 | ENST00000416426.6 | MSI2 | Intron | SCLC-A |
| chr17: 57291119 | ENST00000416426.6 | MSI2 | Intron | SCLC-A |
| chr17: 57291159 | ENST00000416426.6 | MSI2 | Intron | SCLC-A |
| chr17: 57313711 | ENST00000416426.6 | MSI2 | Intron | SCLC-A |
| chr17: 57313728 | ENST00000416426.6 | MSI2 | Intron | SCLC-A |
| chr17: 57313744 | ENST00000416426.6 | MSI2 | Intron | SCLC-A |
| chr17: 57863162 | ENST00000577830.6 | CUEDC1 | Exon | SCLC-A |
| chr17: 57863169 | ENST00000577830.6 | CUEDC1 | Exon | SCLC-A |
| chr17: 72116684 | ENST00000529667.1 | SOX9-AS1 | Intron | SCLC-A |
| chr17: 72122814 | ENST00000628742.2 | SOX9-AS1 | Intron | SCLC-A |
| chr17: 72122816 | ENST00000628742.2 | SOX9-AS1 | Intron | SCLC-A |
| chr17: 72122866 | ENST00000628742.2 | SOX9-AS1 | Intron | SCLC-A |
| chr17: 72122904 | ENST00000628742.2 | SOX9-AS1 | Intron | SCLC-A |
| chr17: 72122914 | ENST00000628742.2 | SOX9-AS1 | Intron | SCLC-A |
| chr17: 72123011 | ENST00000628742.2 | SOX9-AS1 | Intron | SCLC-A |
| chr17: 72123016 | ENST00000628742.2 | SOX9-AS1 | Intron | SCLC-A |
| chr17: 72123038 | ENST00000628742.2 | SOX9-AS1 | Intron | SCLC-A |
| chr17: 74342167 | ENST00000551294.5 | KIF19 | Intron | SCLC-A |
| chr17: 74749702 | ENST00000585285.1 | NA | Promoter | SCLC-A |
| chr17: 74749722 | ENST00000585285.1 | NA | Promoter | SCLC-A |
| chr17: 74749723 | ENST00000585285.1 | NA | Promoter | SCLC-A |
| chr17: 74749726 | ENST00000585285.1 | NA | Promoter | SCLC-A |
| chr17: 74749727 | ENST00000585285.1 | NA | Promoter | SCLC-A |
| chr17: 74749783 | ENST00000585285.1 | NA | Promoter | SCLC-A |
| chr17: 76500832 | ENST00000585989.5 | RHBDF2 | Intron | SCLC-A |
| chr17: 76500871 | ENST00000585989.5 | RHBDF2 | Intron | SCLC-A |
| chr17: 76500989 | ENST00000585989.5 | RHBDF2 | Intron | SCLC-A |
| chr17: 76500995 | ENST00000585989.5 | RHBDF2 | Intron | SCLC-A |
| chr17: 76501008 | ENST00000585989.5 | RHBDF2 | Intron | SCLC-A |
| chr17: 76707050 | ENST00000355797.7 | MXRA7 | Intron | SCLC-A |
| chr17: 76707068 | ENST00000355797.7 | MXRA7 | Intron | SCLC-A |
| chr17: 77147112 | ENST00000589827.5 | SEC14L1 | Intron | SCLC-A |
| chr17: 77147117 | ENST00000589827.5 | SEC14L1 | Intron | SCLC-A |
| chr17: 77147125 | ENST00000589827.5 | SEC14L1 | Intron | SCLC-A |
| chr17: 77147254 | ENST00000589827.5 | SEC14L1 | Intron | SCLC-A |
| chr17: 77209273 | ENST00000392476.6 | SEC14L1 | Intron | SCLC-A |
| chr17: 77209289 | ENST00000392476.6 | SEC14L1 | Intron | SCLC-A |
| chr17: 77376297 | ENST00000589070.1 | SEPTIN9 | Intron | SCLC-A |
| chr17: 77376327 | ENST00000589070.1 | SEPTIN9 | Intron | SCLC-A |
| chr17: 77376328 | ENST00000589070.1 | SEPTIN9 | Intron | SCLC-A |
| chr17: 77376339 | ENST00000589070.1 | SEPTIN9 | Intron | SCLC-A |
| chr17: 77376342 | ENST00000589070.1 | SEPTIN9 | Intron | SCLC-A |
| chr17: 77376365 | ENST00000589070.1 | SEPTIN9 | Intron | SCLC-A |
| chr17: 79465822 | ENST00000583458.5 | RBFOX3 | Intron | SCLC-A |
| chr17: 81171171 | ENST00000414089.5 | PVALEF | Intron | SCLC-A |
| chr17: 81171258 | ENST00000414089.5 | PVALEF | Intron | SCLC-A |
| chr18: 499383 | ENST00000400256.5 | COLEC12 | Intron | SCLC-A |
| chr18: 5890339 | ENST00000581347.2 | TMEM200C | Exon | SCLC-A |
| chr18: 5890342 | ENST00000581347.2 | TMEM200C | Exon | SCLC-A |
| chr18: 5890354 | ENST00000581347.2 | TMEM200C | Exon | SCLC-A |
| chr18: 5890365 | ENST00000581347.2 | TMEM200C | Exon | SCLC-A |
| chr18: 5890371 | ENST00000581347.2 | TMEM200C | Exon | SCLC-A |
| chr18: 74900924 | ENST00000577538.5 | ZNF407 | Intron | SCLC-A |
| chr18: 74900968 | ENST00000577538.5 | ZNF407 | Intron | SCLC-A |
| chr18: 74902241 | ENST00000577538.5 | ZNF407 | Intron | SCLC-A |
| chr18: 74902300 | ENST00000577538.5 | ZNF407 | Intron | SCLC-A |
| chr18: 74902301 | ENST00000577538.5 | ZNF407 | Intron | SCLC-A |
| chr18: 74902320 | ENST00000577538.5 | ZNF407 | Intron | SCLC-A |
| chr18: 74902321 | ENST00000577538.5 | ZNF407 | Intron | SCLC-A |
| chr18: 74902335 | ENST00000577538.5 | ZNF407 | Intron | SCLC-A |
| chr18: 74902336 | ENST00000577538.5 | ZNF407 | Intron | SCLC-A |
| chr18: 74902342 | ENST00000577538.5 | ZNF407 | Intron | SCLC-A |
| chr18: 74902343 | ENST00000577538.5 | ZNF407 | Intron | SCLC-A |
| chr18: 74902365 | ENST00000577538.5 | ZNF407 | Intron | SCLC-A |
| chr18: 75455550 | ENST00000657893.1 | NA | Promoter | SCLC-A |
| chr18: 75455569 | ENST00000657893.1 | NA | Promoter | SCLC-A |
| chr18: 75455571 | ENST00000657893.1 | NA | Promoter | SCLC-A |
| chr18: 80040002 | ENST00000569722.5 | RBFA | Intron | SCLC-A |
| chr19: 427239 | ENST00000590170.3 | SHC2 | Intron | SCLC-A |
| chr19: 427240 | ENST00000590170.3 | SHC2 | Intron | SCLC-A |
| chr19: 427245 | ENST00000590170.3 | SHC2 | Intron | SCLC-A |
| chr19: 427258 | ENST00000590170.3 | SHC2 | Intron | SCLC-A |
| chr19: 1470256 | ENST00000588427.5 | C19orf25 | Intron | SCLC-A |
| chr19: 1470259 | ENST00000588427.5 | C19orf25 | Intron | SCLC-A |
| chr19: 1470285 | ENST00000588427.5 | C19orf25 | Intron | SCLC-A |
| chr19: 6074027 | ENST00000589742.5 | RFX2 | Intron | SCLC-A |
| chr19: 6074028 | ENST00000589742.5 | RFX2 | Intron | SCLC-A |
| chr19: 6074113 | ENST00000589742.5 | RFX2 | Intron | SCLC-A |
| chr19: 6074154 | ENST00000589742.5 | RFX2 | Intron | SCLC-A |
| chr19: 6074157 | ENST00000589742.5 | RFX2 | Intron | SCLC-A |
| chr19: 6074182 | ENST00000589742.5 | RFX2 | Intron | SCLC-A |
| chr19: 6074209 | ENST00000589742.5 | RFX2 | Intron | SCLC-A |
| chr19: 6074210 | ENST00000589742.5 | RFX2 | Intron | SCLC-A |
| chr19: 12754697 | ENST00000549706.5 | BEST2 | Exon | SCLC-A |
| chr19: 12754708 | ENST00000549706.5 | BEST2 | Exon | SCLC-A |
| chr19: 12754725 | ENST00000549706.5 | BEST2 | Exon | SCLC-A |
| chr19: 13593983 | ENST00000574974.3 | CACNA1A | Intron | SCLC-A |
| chr19: 13593987 | ENST00000574974.3 | CACNA1A | Intron | SCLC-A |
| chr19: 14434607 | ENST00000585619.5 | PKN1 | Intron | SCLC-A |
| chr19: 18461133 | ENST00000262809.9 | ELL | Intron | SCLC-A |
| chr19: 18461149 | ENST00000262809.9 | ELL | Intron | SCLC-A |
| chr19: 18743798 | ENST00000321949.13 | CRTC1 | Intron | SCLC-A |
| chr19: 18745079 | ENST00000321949.13 | CRTC1 | Intron | SCLC-A |
| chr19: 33393324 | ENST00000244137.12 | PEPD | Intron | SCLC-A |
| chr19: 33393408 | ENST00000244137.12 | PEPD | Intron | SCLC-A |
| chr19: 33393478 | ENST00000244137.12 | PEPD | Intron | SCLC-A |
| chr19: 35494521 | ENST00000479340.1 | KRTDAP | Intron | SCLC-A |
| chr19: 36111394 | ENST00000585332.3 | OVOL3 | Exon | SCLC-A |
| chr19: 36111668 | ENST00000633214.1 | OVOL3 | Intron | SCLC-A |
| chr19: 38253025 | ENST00000590510.5 | SPINT2 | Intron | SCLC-A |
| chr19: 38253057 | ENST00000590510.5 | SPINT2 | Intron | SCLC-A |
| chr19: 40275139 | ENST00000391844.8 | AKT2 | Intron | SCLC-A |
| chr19: 46001416 | ENST00000536603.5 | CCDC61 | Intron | SCLC-A |
| chr19: 46493529 | ENST00000602017.5 | PPP5D1 | Intron | SCLC-A |
| chr19: 46493540 | ENST00000602017.5 | PPP5D1 | Intron | SCLC-A |
| chr19: 46493562 | ENST00000602017.5 | PPP5D1 | Intron | SCLC-A |
| chr19: 46493583 | ENST00000602017.5 | PPP5D1 | Intron | SCLC-A |
| chr19: 46493592 | ENST00000602017.5 | PPP5D1 | Intron | SCLC-A |
| chr19: 46493606 | ENST00000602017.5 | PPP5D1 | Intron | SCLC-A |
| chr19: 46493631 | ENST00000602017.5 | PPP5D1 | Intron | SCLC-A |
| chr19: 48728959 | ENST00000599291.1 | RASIP1 | Intron | SCLC-A |
| chr19: 48729058 | ENST00000599291.1 | RASIP1 | Intron | SCLC-A |
| chr19: 48729120 | ENST00000599291.1 | RASIP1 | Intron | SCLC-A |
| chr19: 48729133 | ENST00000599291.1 | RASIP1 | Intron | SCLC-A |
| chr19: 48729248 | ENST00000599291.1 | RASIP1 | Intron | SCLC-A |
| chr19: 48729249 | ENST00000599291.1 | RASIP1 | Intron | SCLC-A |
| chr19: 49104754 | ENST00000601065.5 | SNRNP70 | Intron | SCLC-A |
| chr19: 50240302 | ENST00000598205.5 | MYH14 | Intron | SCLC-A |
| chr19: 50276060 | ENST00000440075.6 | MYH14 | UTR5 | SCLC-A |
| chr19: 50543108 | ENST00000599957.5 | LRRC4B | Intron | SCLC-A |
| chr19: 55359800 | ENST00000585734.5 | FAM71E2 | Exon | SCLC-A |
| chr19: 56217428 | ENST00000474329.2 | NA | Exon | SCLC-A |
| chr19: 56217452 | ENST00000474329.2 | NA | Exon | SCLC-A |
| chr19: 56217478 | ENST00000474329.2 | NA | Exon | SCLC-A |
| chr19: 56217554 | ENST00000474329.2 | NA | Exon | SCLC-A |
| chr19: 56217563 | ENST00000474329.2 | NA | Exon | SCLC-A |
| chr19: 56217567 | ENST00000474329.2 | NA | Exon | SCLC-A |
| chr19: 56217571 | ENST00000474329.2 | NA | Exon | SCLC-A |
| chr19: 56217718 | ENST00000474329.2 | NA | Exon | SCLC-A |
| chr19: 56217723 | ENST00000474329.2 | NA | Exon | SCLC-A |
| chr19: 56217743 | ENST00000474329.2 | NA | Exon | SCLC-A |
| chr19: 56217762 | ENST00000474329.2 | NA | Exon | SCLC-A |
| chr19: 56217775 | ENST00000474329.2 | NA | Exon | SCLC-A |
| chr19: 56217798 | ENST00000474329.2 | NA | Promoter | SCLC-A |
| chr19: 56217840 | ENST00000474329.2 | NA | Promoter | SCLC-A |
| chr19: 56217842 | ENST00000474329.2 | NA | Promoter | SCLC-A |
| chr19: 56217844 | ENST00000474329.2 | NA | Promoter | SCLC-A |
| chr19: 56217846 | ENST00000474329.2 | NA | Promoter | SCLC-A |
| chr2: 1680504 | ENST00000252804.9 | PXDN | Intron | SCLC-A |
| chr2: 6958060 | ENST00000467276.5 | RNF144A | Intron | SCLC-A |
| chr2: 7020509 | ENST00000467276.5 | RNF144A | Exon | SCLC-A |
| chr2: 8503048 | ENST00000670008.1 | LINC01814 | Intron | SCLC-A |
| chr2: 8503070 | ENST00000670008.1 | LINC01814 | Intron | SCLC-A |
| chr2: 8528861 | ENST00000670008.1 | LINC01814 | Intron | SCLC-A |
| chr2: 8545546 | ENST00000670008.1 | LINC01814 | Intron | SCLC-A |
| chr2: 8676641 | ENST00000455965.6 | ID2-AS1 | Intron | SCLC-A |
| chr2: 8676643 | ENST00000455965.6 | ID2-AS1 | Intron | SCLC-A |
| chr2: 8676714 | ENST00000455965.6 | ID2-AS1 | Intron | SCLC-A |
| chr2: 8676725 | ENST00000455965.6 | ID2-AS1 | Intron | SCLC-A |
| chr2: 8676733 | ENST00000455965.6 | ID2-AS1 | Intron | SCLC-A |
| chr2: 8676787 | ENST00000455965.6 | ID2-AS1 | Intron | SCLC-A |
| chr2: 8676789 | ENST00000455965.6 | ID2-AS1 | Intron | SCLC-A |
| chr2: 8676793 | ENST00000455965.6 | ID2-AS1 | Intron | SCLC-A |
| chr2: 8676854 | ENST00000455965.6 | ID2-AS1 | Intron | SCLC-A |
| chr2: 9379004 | ENST00000315273.4 | ASAP2 | Exon | SCLC-A |
| chr2: 9379035 | ENST00000315273.4 | ASAP2 | Exon | SCLC-A |
| chr2: 10911362 | ENST00000295082.3 | KCNF1 | Promoter | SCLC-A |
| chr2: 11677228 | ENST00000449576.6 | LPIN1 | Promoter | SCLC-A |
| chr2: 11677265 | ENST00000449576.6 | LPIN1 | Promoter | SCLC-A |
| chr2: 11677266 | ENST00000449576.6 | LPIN1 | Promoter | SCLC-A |
| chr2: 11677662 | ENST00000617634.1 | NA | Promoter | SCLC-A |
| chr2: 11677663 | ENST00000617634.1 | NA | Promoter | SCLC-A |
| chr2: 11677669 | ENST00000617634.1 | NA | Promoter | SCLC-A |
| chr2: 11697772 | ENST00000449576.6 | LPIN1 | Intron | SCLC-A |
| chr2: 19125197 | ENST00000449124.1 | NA | Intron | SCLC-A |
| chr2: 23563598 | ENST00000486442.6 | KLHL29 | Intron | SCLC-A |
| chr2: 23563632 | ENST00000486442.6 | KLHL29 | Intron | SCLC-A |
| chr2: 23563634 | ENST00000486442.6 | KLHL29 | Intron | SCLC-A |
| chr2: 23656640 | ENST00000486442.6 | KLHL29 | Intron | SCLC-A |
| chr2: 23656658 | ENST00000486442.6 | KLHL29 | Intron | SCLC-A |
| chr2: 23656731 | ENST00000486442.6 | KLHL29 | Intron | SCLC-A |
| chr2: 23656732 | ENST00000486442.6 | KLHL29 | Intron | SCLC-A |
| chr2: 23656760 | ENST00000486442.6 | KLHL29 | Intron | SCLC-A |
| chr2: 23656805 | ENST00000486442.6 | KLHL29 | Intron | SCLC-A |
| chr2: 24010805 | ENST00000406420.7 | MFSD2B | Intron | SCLC-A |
| chr2: 24010822 | ENST00000406420.7 | MFSD2B | Intron | SCLC-A |
| chr2: 24010887 | ENST00000406420.7 | MFSD2B | Intron | SCLC-A |
| chr2: 24010950 | ENST00000406420.7 | MFSD2B | Intron | SCLC-A |
| chr2: 24886737 | ENST00000435135.5 | ADCY3 | Intron | SCLC-A |
| chr2: 24886746 | ENST00000435135.5 | ADCY3 | Intron | SCLC-A |
| chr2: 24886769 | ENST00000435135.5 | ADCY3 | Intron | SCLC-A |
| chr2: 24886961 | ENST00000435135.5 | ADCY3 | Intron | SCLC-A |
| chr2: 25419501 | ENST00000496972.6 | DTNB | Intron | SCLC-A |
| chr2: 25419709 | ENST00000496972.6 | DTNB | Intron | SCLC-A |
| chr2: 34051135 | ENST00000366209.6 | LINC01317 | Intron | SCLC-A |
| chr2: 34289711 | ENST00000650021.1 | LINC01320 | Intron | SCLC-A |
| chr2: 43224560 | ENST00000282388.4 | ZFP36L2 | Exon | SCLC-A |
| chr2: 43224562 | ENST00000282388.4 | ZFP36L2 | Exon | SCLC-A |
| chr2: 43224575 | ENST00000282388.4 | ZFP36L2 | Exon | SCLC-A |
| chr2: 43224881 | ENST00000282388.4 | ZFP36L2 | Exon | SCLC-A |
| chr2: 43225335 | ENST00000282388.4 | ZFP36L2 | Exon | SCLC-A |
| chr2: 43225336 | ENST00000282388.4 | ZFP36L2 | Exon | SCLC-A |
| chr2: 43225349 | ENST00000282388.4 | ZFP36L2 | Exon | SCLC-A |
| chr2: 43225369 | ENST00000282388.4 | ZFP36L2 | Exon | SCLC-A |
| chr2: 46983312 | ENST00000409245.5 | TTC7A | Intron | SCLC-A |
| chr2: 46983348 | ENST00000409245.5 | TTC7A | Intron | SCLC-A |
| chr2: 46983349 | ENST00000409245.5 | TTC7A | Intron | SCLC-A |
| chr2: 46983376 | ENST00000409245.5 | TTC7A | Intron | SCLC-A |
| chr2: 46983377 | ENST00000409245.5 | TTC7A | Intron | SCLC-A |
| chr2: 46983413 | ENST00000409245.5 | TTC7A | Intron | SCLC-A |
| chr2: 46983446 | ENST00000409245.5 | TTC7A | Intron | SCLC-A |
| chr2: 46983461 | ENST00000409245.5 | TTC7A | Intron | SCLC-A |
| chr2: 47370310 | ENST00000405271.5 | EPCAM | Intron | SCLC-A |
| chr2: 47370334 | ENST00000405271.5 | EPCAM | Intron | SCLC-A |
| chr2: 47370340 | ENST00000405271.5 | EPCAM | Intron | SCLC-A |
| chr2: 63051505 | ENST00000477348.1 | OTX1 | Exon | SCLC-A |
| chr2: 67561566 | ENST00000433065.1 | NA | Promoter | SCLC-A |
| chr2: 67561575 | ENST00000433065.1 | NA | Promoter | SCLC-A |
| chr2: 67561705 | ENST00000433065.1 | NA | Promoter | SCLC-A |
| chr2: 96143896 | ENST00000288943.5 | DUSP2 | Exon | SCLC-A |
| chr2: 96143899 | ENST00000288943.5 | DUSP2 | Exon | SCLC-A |
| chr2: 96143909 | ENST00000288943.5 | DUSP2 | Exon | SCLC-A |
| chr2: 96143924 | ENST00000288943.5 | DUSP2 | Exon | SCLC-A |
| chr2: 96143932 | ENST00000288943.5 | DUSP2 | Exon | SCLC-A |
| chr2: 96143938 | ENST00000288943.5 | DUSP2 | Exon | SCLC-A |
| chr2: 96143941 | ENST00000288943.5 | DUSP2 | Exon | SCLC-A |
| chr2: 96143981 | ENST00000288943.5 | DUSP2 | Exon | SCLC-A |
| chr2: 96143983 | ENST00000288943.5 | DUSP2 | Exon | SCLC-A |
| chr2: 96143992 | ENST00000288943.5 | DUSP2 | Exon | SCLC-A |
| chr2: 96144014 | ENST00000288943.5 | DUSP2 | Exon | SCLC-A |
| chr2: 98357101 | ENST00000393504.5 | CNGA3 | Intron | SCLC-A |
| chr2: 109482629 | ENST00000309415.8 | SH3RF3 | Intron | SCLC-A |
| chr2: 109482630 | ENST00000309415.8 | SH3RF3 | Intron | SCLC-A |
| chr2: 109498112 | ENST00000309415.8 | SH3RF3 | Intron | SCLC-A |
| chr2: 109498141 | ENST00000309415.8 | SH3RF3 | Intron | SCLC-A |
| chr2: 109498160 | ENST00000309415.8 | SH3RF3 | Intron | SCLC-A |
| chr2: 109498161 | ENST00000309415.8 | SH3RF3 | Intron | SCLC-A |
| chr2: 109498193 | ENST00000309415.8 | SH3RF3 | Intron | SCLC-A |
| chr2: 109498196 | ENST00000309415.8 | SH3RF3 | Intron | SCLC-A |
| chr2: 127071160 | ENST00000376113.6 | BIN1 | Intron | SCLC-A |
| chr2: 131528743 | ENST00000434330.1 | CCDC74A | Promoter | SCLC-A |
| chr2: 131528787 | ENST00000434330.1 | CCDC74A | Promoter | SCLC-A |
| chr2: 131528898 | ENST00000434330.1 | CCDC74A | Promoter | SCLC-A |
| chr2: 144404070 | ENST00000638087.1 | ZEB2 | Exon | SCLC-A |
| chr2: 166610661 | ENST00000650747.1 | SCN7A | Intron | SCLC-A |
| chr2: 166610663 | ENST00000650747.1 | SCN7A | Intron | SCLC-A |
| chr2: 166610666 | ENST00000650747.1 | SCN7A | Intron | SCLC-A |
| chr2: 166610700 | ENST00000650747.1 | SCN7A | Intron | SCLC-A |
| chr2: 181656516 | ENST00000479558.5 | CERKL | Intron | SCLC-A |
| chr2: 181656577 | ENST00000479558.5 | CERKL | Intron | SCLC-A |
| chr2: 237508969 | ENST00000422695.5 | MLPH | Intron | SCLC-A |
| chr20: 11247539 | ENST00000444792.1 | LOC339593 | Intron | SCLC-A |
| chr20: 11271495 | ENST00000666460.1 | LOC339593 | Intron | SCLC-A |
| chr20: 11271529 | ENST00000666460.1 | LOC339593 | Intron | SCLC-A |
| chr20: 19759038 | ENST00000432334.2 | RIN2 | Intron | SCLC-A |
| chr20: 20562289 | ENST00000202677.12 | RALGAPA2 | Intron | SCLC-A |
| chr20: 20562488 | ENST00000202677.12 | RALGAPA2 | Intron | SCLC-A |
| chr20: 20562510 | ENST00000202677.12 | RALGAPA2 | Intron | SCLC-A |
| chr20: 21101464 | ENST00000655093.1 | LINC00237 | Intron | SCLC-A |
| chr20: 21101552 | ENST00000655093.1 | LINC00237 | Intron | SCLC-A |
| chr20: 21101558 | ENST00000655093.1 | LINC00237 | Intron | SCLC-A |
| chr20: 21101575 | ENST00000655093.1 | LINC00237 | Intron | SCLC-A |
| chr20: 23084938 | ENST00000246006.5 | CD93 | Exon | SCLC-A |
| chr20: 23084947 | ENST00000246006.5 | CD93 | Exon | SCLC-A |
| chr20: 23084948 | ENST00000246006.5 | CD93 | Exon | SCLC-A |
| chr20: 23085004 | ENST00000246006.5 | CD93 | Exon | SCLC-A |
| chr20: 23085011 | ENST00000246006.5 | CD93 | Exon | SCLC-A |
| chr20: 23085015 | ENST00000246006.5 | CD93 | Exon | SCLC-A |
| chr20: 23085064 | ENST00000246006.5 | CD93 | Exon | SCLC-A |
| chr20: 23085069 | ENST00000246006.5 | CD93 | Exon | SCLC-A |
| chr20: 32574566 | ENST00000621426.6 | NOL4L | Intron | SCLC-A |
| chr20: 32581433 | ENST00000621426.6 | NOL4L | Intron | SCLC-A |
| chr20: 32676689 | ENST00000646357.1 | C20orf203 | Intron | SCLC-A |
| chr20: 32676733 | ENST00000646357.1 | C20orf203 | Intron | SCLC-A |
| chr20: 36471322 | ENST00000373913.7 | DLGAP4 | Intron | SCLC-A |
| chr20: 36471323 | ENST00000373913.7 | DLGAP4 | Intron | SCLC-A |
| chr20: 36471351 | ENST00000373913.7 | DLGAP4 | Intron | SCLC-A |
| chr20: 36529602 | ENST00000439595.5 | DLGAP4-AS1 | Intron | SCLC-A |
| chr20: 36852377 | ENST00000237536.9 | SOGA1 | Intron | SCLC-A |
| chr20: 53595418 | ENST00000431687.5 | NA | Intron | SCLC-A |
| chr20: 53595431 | ENST00000431687.5 | NA | Intron | SCLC-A |
| chr20: 60310498 | ENST00000667664.1 | MIR646HG | Intron | SCLC-A |
| chr20: 61317297 | ENST00000614565.5 | CDH4 | Intron | SCLC-A |
| chr20: 63065670 | ENST00000606208.5 | LINC01749 | Intron | SCLC-A |
| chr20: 63065682 | ENST00000606208.5 | LINC01749 | Intron | SCLC-A |
| chr20: 63069611 | ENST00000606208.5 | LINC01749 | Intron | SCLC-A |
| chr21: 38661456 | ENST00000398910.5 | ERG | Intron | SCLC-A |
| chr21: 38661498 | ENST00000398910.5 | ERG | Intron | SCLC-A |
| chr21: 38661510 | ENST00000398910.5 | ERG | Intron | SCLC-A |
| chr21: 38661526 | ENST00000398910.5 | ERG | Intron | SCLC-A |
| chr21: 38661529 | ENST00000398910.5 | ERG | Intron | SCLC-A |
| chr21: 38661621 | ENST00000398910.5 | ERG | Intron | SCLC-A |
| chr21: 38661628 | ENST00000398910.5 | ERG | Intron | SCLC-A |
| chr21: 38661637 | ENST00000398910.5 | ERG | Intron | SCLC-A |
| chr21: 38661638 | ENST00000398910.5 | ERG | Intron | SCLC-A |
| chr21: 38661664 | ENST00000398919.6 | ERG | Promoter | SCLC-A |
| chr21: 38661689 | ENST00000442448.5 | ERG | UTR5 | SCLC-A |
| chr21: 38661696 | ENST00000442448.5 | ERG | UTR5 | SCLC-A |
| chr21: 38661697 | ENST00000442448.5 | ERG | UTR5 | SCLC-A |
| chr21: 38661701 | ENST00000442448.5 | ERG | UTR5 | SCLC-A |
| chr21: 38661705 | ENST00000442448.5 | ERG | UTR5 | SCLC-A |
| chr21: 38661706 | ENST00000442448.5 | ERG | UTR5 | SCLC-A |
| chr21: 41835462 | ENST00000447016.6 | PRDM15 | Exon | SCLC-A |
| chr21: 41835464 | ENST00000447016.6 | PRDM15 | Exon | SCLC-A |
| chr21: 43936339 | ENST00000291572.13 | AGPAT3 | Intron | SCLC-A |
| chr21: 43936375 | ENST00000291572.13 | AGPAT3 | Intron | SCLC-A |
| chr21: 43936379 | ENST00000291572.13 | AGPAT3 | Intron | SCLC-A |
| chr21: 43936441 | ENST00000291572.13 | AGPAT3 | Intron | SCLC-A |
| chr21: 43936460 | ENST00000291572.13 | AGPAT3 | Intron | SCLC-A |
| chr21: 44508171 | ENST00000323084.9 | TSPEAR | Intron | SCLC-A |
| chr21: 44508179 | ENST00000323084.9 | TSPEAR | Intron | SCLC-A |
| chr21: 44508206 | ENST00000323084.9 | TSPEAR | Intron | SCLC-A |
| chr21: 44518949 | ENST00000323084.9 | TSPEAR | Intron | SCLC-A |
| chr21: 44518997 | ENST00000323084.9 | TSPEAR | Intron | SCLC-A |
| chr21: 44521454 | ENST00000323084.9 | TSPEAR | Intron | SCLC-A |
| chr21: 44522598 | ENST00000465978.1 | TSPEAR-AS2 | Intron | SCLC-A |
| chr21: 44525864 | ENST00000465978.1 | TSPEAR-AS2 | Exon | SCLC-A |
| chr21: 44525881 | ENST00000465978.1 | TSPEAR-AS2 | Exon | SCLC-A |
| chr21: 44525890 | ENST00000465978.1 | TSPEAR-AS2 | Exon | SCLC-A |
| chr21: 44525980 | ENST00000323084.9 | TSPEAR | Intron | SCLC-A |
| chr21: 44570184 | ENST00000323084.9 | TSPEAR | Intron | SCLC-A |
| chr21: 44589901 | ENST00000323084.9 | TSPEAR | Intron | SCLC-A |
| chr21: 44589918 | ENST00000323084.9 | TSPEAR | Intron | SCLC-A |
| chr21: 44589919 | ENST00000323084.9 | TSPEAR | Intron | SCLC-A |
| chr21: 44590033 | ENST00000323084.9 | TSPEAR | Intron | SCLC-A |
| chr21: 44590034 | ENST00000323084.9 | TSPEAR | Intron | SCLC-A |
| chr21: 44590045 | ENST00000323084.9 | TSPEAR | Intron | SCLC-A |
| chr21: 44602659 | ENST00000323084.9 | TSPEAR | Intron | SCLC-A |
| chr21: 44639620 | ENST00000323084.9 | TSPEAR | Intron | SCLC-A |
| chr21: 44649130 | ENST00000323084.9 | TSPEAR | Intron | SCLC-A |
| chr21: 44658852 | ENST00000323084.9 | TSPEAR | Intron | SCLC-A |
| chr21: 44698147 | ENST00000323084.9 | TSPEAR | Intron | SCLC-A |
| chr21: 44698191 | ENST00000323084.9 | TSPEAR | Intron | SCLC-A |
| chr21: 44698714 | ENST00000323084.9 | TSPEAR | Intron | SCLC-A |
| chr21: 44698723 | ENST00000323084.9 | TSPEAR | Intron | SCLC-A |
| chr21: 44705577 | ENST00000323084.9 | TSPEAR | Intron | SCLC-A |
| chr22: 31579558 | ENST00000524296.5 | SFI1 | Intron | SCLC-A |
| chr22: 31579562 | ENST00000524296.5 | SFI1 | Intron | SCLC-A |
| chr22: 31579567 | ENST00000524296.5 | SFI1 | Intron | SCLC-A |
| chr22: 31579595 | ENST00000524296.5 | SFI1 | Intron | SCLC-A |
| chr22: 31579607 | ENST00000524296.5 | SFI1 | Intron | SCLC-A |
| chr22: 31579611 | ENST00000524296.5 | SFI1 | Intron | SCLC-A |
| chr22: 36816302 | ENST00000216200.9 | PVALB | Intron | SCLC-A |
| chr22: 36816346 | ENST00000216200.9 | PVALB | Intron | SCLC-A |
| chr22: 36816347 | ENST00000216200.9 | PVALB | Intron | SCLC-A |
| chr22: 36816387 | ENST00000216200.9 | PVALB | Intron | SCLC-A |
| chr22: 36816390 | ENST00000216200.9 | PVALB | Intron | SCLC-A |
| chr22: 36816391 | ENST00000216200.9 | PVALB | Intron | SCLC-A |
| chr22: 36816410 | ENST00000216200.9 | PVALB | Intron | SCLC-A |
| chr22: 36816411 | ENST00000216200.9 | PVALB | Intron | SCLC-A |
| chr22: 36861229 | ENST00000619915.1 | NCF4-AS1 | Intron | SCLC-A |
| chr22: 37485353 | ENST00000438891.5 | MFNG | Intron | SCLC-A |
| chr22: 37485696 | ENST00000438891.5 | MFNG | Intron | SCLC-A |
| chr22: 37486040 | ENST00000416983.7 | MFNG | Exon | SCLC-A |
| chr22: 37486046 | ENST00000416983.7 | MFNG | Exon | SCLC-A |
| chr22: 37486082 | ENST00000416983.7 | MFNG | Exon | SCLC-A |
| chr22: 42320519 | ENST00000515426.1 | TCF20 | Intron | SCLC-A |
| chr22: 42320716 | ENST00000515426.1 | TCF20 | Intron | SCLC-A |
| chr22: 42431587 | ENST00000329021.9 | NFAM1 | Intron | SCLC-A |
| chr22: 42431594 | ENST00000329021.9 | NFAM1 | Intron | SCLC-A |
| chr22: 48871054 | ENST00000380981.3 | LINC01310 | Intron | SCLC-A |
| chr22: 48871068 | ENST00000380981.3 | LINC01310 | Intron | SCLC-A |
| chr22: 48871084 | ENST00000380981.3 | LINC01310 | Intron | SCLC-A |
| chr22: 48871088 | ENST00000380981.3 | LINC01310 | Intron | SCLC-A |
| chr22: 48871168 | ENST00000380981.3 | LINC01310 | Intron | SCLC-A |
| chr22: 50049855 | ENST00000266182.10 | TTLL8 | Intron | SCLC-A |
| chr3: 16842354 | ENST00000460467.1 | PLCL2 | Intron | SCLC-A |
| chr3: 16842392 | ENST00000460467.1 | PLCL2 | Intron | SCLC-A |
| chr3: 27126857 | ENST00000295720.10 | NEK10 | Intron | SCLC-A |
| chr3: 27126864 | ENST00000295720.10 | NEK10 | Intron | SCLC-A |
| chr3: 45594551 | ENST00000465039.5 | LIMD1 | Intron | SCLC-A |
| chr3: 45594698 | ENST00000465039.5 | LIMD1 | Intron | SCLC-A |
| chr3: 45594699 | ENST00000465039.5 | LIMD1 | Intron | SCLC-A |
| chr3: 45796331 | ENST00000358525.9 | SLC6A20 | Exon | SCLC-A |
| chr3: 45796338 | ENST00000358525.9 | SLC6A20 | Exon | SCLC-A |
| chr3: 45796353 | ENST00000358525.9 | SLC6A20 | Exon | SCLC-A |
| chr3: 45796356 | ENST00000358525.9 | SLC6A20 | Exon | SCLC-A |
| chr3: 45796360 | ENST00000358525.9 | SLC6A20 | Exon | SCLC-A |
| chr3: 45915943 | ENST00000418700.5 | LZTFL1 | Exon | SCLC-A |
| chr3: 53167135 | ENST00000478843.5 | PRKCD | Intron | SCLC-A |
| chr3: 55702453 | ENST00000288221.11 | ERC2 | Intron | SCLC-A |
| chr3: 55702593 | ENST00000288221.11 | ERC2 | Intron | SCLC-A |
| chr3: 120284773 | ENST00000464295.5 | GPR156 | Intron | SCLC-A |
| chr3: 183179235 | ENST00000328913.7 | MCF2L2 | Exon | SCLC-A |
| chr3: 183179343 | ENST00000328913.7 | MCF2L2 | Exon | SCLC-A |
| chr3: 183179354 | ENST00000328913.7 | MCF2L2 | Exon | SCLC-A |
| chr3: 183179361 | ENST00000328913.7 | MCF2L2 | Exon | SCLC-A |
| chr3: 183179371 | ENST00000328913.7 | MCF2L2 | Exon | SCLC-A |
| chr3: 183179395 | ENST00000328913.7 | MCF2L2 | Exon | SCLC-A |
| chr3: 183179435 | ENST00000328913.7 | MCF2L2 | Exon | SCLC-A |
| chr3: 183179438 | ENST00000328913.7 | MCF2L2 | Exon | SCLC-A |
| chr3: 183179812 | ENST00000328913.7 | MCF2L2 | Intron | SCLC-A |
| chr3: 187743254 | ENST00000621333.4 | BCL6 | Intron | SCLC-A |
| chr3: 187743320 | ENST00000621333.4 | BCL6 | Intron | SCLC-A |
| chr3: 187743335 | ENST00000621333.4 | BCL6 | Intron | SCLC-A |
| chr3: 194305677 | ENST00000654752.1 | LINC00887 | Intron | SCLC-A |
| chr4: 5807717 | ENST00000506216.5 | CRMP1 | Intron | SCLC-A |
| chr4: 5807729 | ENST00000506216.5 | CRMP1 | Intron | SCLC-A |
| chr4: 6957500 | ENST00000448507.5 | TBC1D14 | Intron | SCLC-A |
| chr4: 6957537 | ENST00000448507.5 | TBC1D14 | Intron | SCLC-A |
| chr4: 7335853 | ENST00000507866.6 | SORCS2 | Intron | SCLC-A |
| chr4: 7335859 | ENST00000507866.6 | SORCS2 | Intron | SCLC-A |
| chr4: 7335860 | ENST00000507866.6 | SORCS2 | Intron | SCLC-A |
| chr4: 8189669 | ENST00000509119.5 | SH3TC1 | Intron | SCLC-A |
| chr4: 8189818 | ENST00000509119.5 | SH3TC1 | Intron | SCLC-A |
| chr4: 8189821 | ENST00000509119.5 | SH3TC1 | Intron | SCLC-A |
| chr4: 8189832 | ENST00000509119.5 | SH3TC1 | Intron | SCLC-A |
| chr4: 52750883 | ENST00000650700.1 | RASL11B | Intron | SCLC-A |
| chr4: 52750910 | ENST00000650700.1 | RASL11B | Intron | SCLC-A |
| chr4: 52750953 | ENST00000650700.1 | RASL11B | Intron | SCLC-A |
| chr4: 52750961 | ENST00000650700.1 | RASL11B | Intron | SCLC-A |
| chr4: 52750966 | ENST00000650700.1 | RASL11B | Intron | SCLC-A |
| chr4: 52750975 | ENST00000650700.1 | RASL11B | Intron | SCLC-A |
| chr4: 52751018 | ENST00000650700.1 | RASL11B | Intron | SCLC-A |
| chr4: 52751021 | ENST00000650700.1 | RASL11B | Intron | SCLC-A |
| chr4: 52751027 | ENST00000650700.1 | RASL11B | Intron | SCLC-A |
| chr4: 52751034 | ENST00000650700.1 | RASL11B | Intron | SCLC-A |
| chr4: 52751037 | ENST00000650700.1 | RASL11B | Intron | SCLC-A |
| chr4: 52751038 | ENST00000650700.1 | RASL11B | Intron | SCLC-A |
| chr4: 52751045 | ENST00000650700.1 | RASL11B | Intron | SCLC-A |
| chr4: 52751058 | ENST00000650700.1 | RASL11B | Intron | SCLC-A |
| chr4: 52751061 | ENST00000650700.1 | RASL11B | Intron | SCLC-A |
| chr4: 52751085 | ENST00000650700.1 | RASL11B | Intron | SCLC-A |
| chr4: 52751101 | ENST00000650700.1 | RASL11B | Intron | SCLC-A |
| chr4: 140100143 | ENST00000502696.1 | MAML3 | Intron | SCLC-A |
| chr4: 140100232 | ENST00000502696.1 | MAML3 | Intron | SCLC-A |
| chr4: 140100233 | ENST00000502696.1 | MAML3 | Intron | SCLC-A |
| chr4: 140100239 | ENST00000502696.1 | MAML3 | Intron | SCLC-A |
| chr4: 159676788 | ENST00000513718.2 | LINC02233 | Intron | SCLC-A |
| chr4: 159676802 | ENST00000513718.2 | LINC02233 | Intron | SCLC-A |
| chr4: 159676803 | ENST00000513718.2 | LINC02233 | Intron | SCLC-A |
| chr4: 159676810 | ENST00000513718.2 | LINC02233 | Intron | SCLC-A |
| chr4: 159676819 | ENST00000513718.2 | LINC02233 | Intron | SCLC-A |
| chr4: 159733646 | ENST00000513718.2 | LINC02233 | Intron | SCLC-A |
| chr5: 1219558 | ENST00000515652.5 | SLC6A19 | Exon | SCLC-A |
| chr5: 64714761 | ENST00000509189.5 | SHISAL2B | Intron | SCLC-A |
| chr5: 64714782 | ENST00000509189.5 | SHISAL2B | Intron | SCLC-A |
| chr5: 64714797 | ENST00000509189.5 | SHISAL2B | Intron | SCLC-A |
| chr5: 64714815 | ENST00000509189.5 | SHISAL2B | Intron | SCLC-A |
| chr5: 64714885 | ENST00000509189.5 | SHISAL2B | Intron | SCLC-A |
| chr5: 64714900 | ENST00000509189.5 | SHISAL2B | Intron | SCLC-A |
| chr5: 64714901 | ENST00000509189.5 | SHISAL2B | Intron | SCLC-A |
| chr5: 64714906 | ENST00000509189.5 | SHISAL2B | Intron | SCLC-A |
| chr5: 64714907 | ENST00000509189.5 | SHISAL2B | Intron | SCLC-A |
| chr5: 64714911 | ENST00000509189.5 | SHISAL2B | Intron | SCLC-A |
| chr5: 72120886 | ENST00000512974.5 | MAP1B | Intron | SCLC-A |
| chr5: 76819971 | ENST00000514165.1 | F2RL1 | Intron | SCLC-A |
| chr5: 76819981 | ENST00000514165.1 | F2RL1 | Intron | SCLC-A |
| chr5: 76819985 | ENST00000514165.1 | F2RL1 | Intron | SCLC-A |
| chr5: 77848149 | ENST00000522370.1 | TBCA | Intron | SCLC-A |
| chr5: 93588608 | ENST00000327111.8 | NR2F1 | Intron | SCLC-A |
| chr5: 95834831 | ENST00000514941.1 | NA | Intron | SCLC-A |
| chr5: 95834835 | ENST00000514941.1 | NA | Intron | SCLC-A |
| chr5: 95834840 | ENST00000514941.1 | NA | Intron | SCLC-A |
| chr5: 95834843 | ENST00000514941.1 | NA | Intron | SCLC-A |
| chr5: 95834898 | ENST00000514941.1 | NA | Intron | SCLC-A |
| chr5: 95834915 | ENST00000514941.1 | NA | Exon | SCLC-A |
| chr5: 95834932 | ENST00000514941.1 | NA | Exon | SCLC-A |
| chr5: 95834935 | ENST00000514941.1 | NA | Exon | SCLC-A |
| chr5: 95834938 | ENST00000514941.1 | NA | Exon | SCLC-A |
| chr5: 135076216 | ENST00000624272.3 | C5orf66 | Intron | SCLC-A |
| chr5: 135076253 | ENST00000624272.3 | C5orf66 | Intron | SCLC-A |
| chr5: 148654157 | ENST00000519495.1 | HTR4 | Intron | SCLC-A |
| chr5: 149271409 | ENST00000522685.1 | NA | Intron | SCLC-A |
| chr5: 149271453 | ENST00000522685.1 | NA | Intron | SCLC-A |
| chr5: 149732251 | ENST00000360453.8 | PPARGC1B | Intron | SCLC-A |
| chr5: 149732314 | ENST00000360453.8 | PPARGC1B | Intron | SCLC-A |
| chr5: 149732315 | ENST00000360453.8 | PPARGC1B | Intron | SCLC-A |
| chr5: 149732320 | ENST00000360453.8 | PPARGC1B | Intron | SCLC-A |
| chr5: 149732321 | ENST00000360453.8 | PPARGC1B | Intron | SCLC-A |
| chr5: 149732340 | ENST00000360453.8 | PPARGC1B | Intron | SCLC-A |
| chr5: 149732365 | ENST00000360453.8 | PPARGC1B | Intron | SCLC-A |
| chr5: 172244917 | ENST00000393792.3 | UBTD2 | Intron | SCLC-A |
| chr5: 172244971 | ENST00000393792.3 | UBTD2 | Intron | SCLC-A |
| chr5: 172244980 | ENST00000393792.3 | UBTD2 | Intron | SCLC-A |
| chr5: 172244981 | ENST00000393792.3 | UBTD2 | Intron | SCLC-A |
| chr6: 25318584 | ENST00000643807.1 | CMAHP | Intron | SCLC-A |
| chr6: 25318617 | ENST00000643807.1 | CMAHP | Intron | SCLC-A |
| chr6: 35469349 | ENST00000467020.5 | RPL10A | Exon | SCLC-A |
| chr6: 35469398 | ENST00000467020.5 | RPL10A | Exon | SCLC-A |
| chr6: 39228312 | ENST00000359534.4 | KCNK5 | Intron | SCLC-A |
| chr6: 39228531 | ENST00000359534.4 | KCNK5 | Intron | SCLC-A |
| chr6: 39228564 | ENST00000359534.4 | KCNK5 | Intron | SCLC-A |
| chr6: 39228565 | ENST00000359534.4 | KCNK5 | Intron | SCLC-A |
| chr6: 39228656 | ENST00000359534.4 | KCNK5 | Intron | SCLC-A |
| chr6: 106046585 | ENST00000652320.1 | PRDM1 | Intron | SCLC-A |
| chr6: 106046699 | ENST00000652320.1 | PRDM1 | Intron | SCLC-A |
| chr6: 107046096 | ENST00000311381.8 | MTRES1 | Intron | SCLC-A |
| chr6: 116371874 | ENST00000430252.6 | DSE | Intron | SCLC-A |
| chr6: 116371906 | ENST00000430252.6 | DSE | Intron | SCLC-A |
| chr6: 134176153 | ENST00000367858.9 | SGK1 | Intron | SCLC-A |
| chr6: 134176164 | ENST00000367858.9 | SGK1 | Intron | SCLC-A |
| chr6: 134176193 | ENST00000367858.9 | SGK1 | Intron | SCLC-A |
| chr6: 134176242 | ENST00000367858.9 | SGK1 | Intron | SCLC-A |
| chr6: 136923105 | ENST00000331858.5 | SLC35D3 | Intron | SCLC-A |
| chr6: 136923373 | ENST00000331858.5 | SLC35D3 | Intron | SCLC-A |
| chr6: 136923389 | ENST00000331858.5 | SLC35D3 | Intron | SCLC-A |
| chr6: 136923893 | ENST00000331858.5 | SLC35D3 | Exon | SCLC-A |
| chr6: 136923901 | ENST00000331858.5 | SLC35D3 | Exon | SCLC-A |
| chr6: 136923905 | ENST00000331858.5 | SLC35D3 | Exon | SCLC-A |
| chr6: 136923920 | ENST00000331858.5 | SLC35D3 | Exon | SCLC-A |
| chr6: 136923925 | ENST00000331858.5 | SLC35D3 | Exon | SCLC-A |
| chr6: 136923936 | ENST00000331858.5 | SLC35D3 | Exon | SCLC-A |
| chr6: 136923937 | ENST00000331858.5 | SLC35D3 | Exon | SCLC-A |
| chr6: 136923950 | ENST00000331858.5 | SLC35D3 | Exon | SCLC-A |
| chr6: 157990685 | ENST00000355585.9 | SYNJ2 | Intron | SCLC-A |
| chr6: 157990693 | ENST00000355585.9 | SYNJ2 | Intron | SCLC-A |
| chr6: 157990742 | ENST00000355585.9 | SYNJ2 | Intron | SCLC-A |
| chr7: 2524983 | ENST00000402506.5 | LFNG | Intron | SCLC-A |
| chr7: 2525019 | ENST00000402506.5 | LFNG | Intron | SCLC-A |
| chr7: 2525025 | ENST00000402506.5 | LFNG | Intron | SCLC-A |
| chr7: 2525181 | ENST00000402506.5 | LFNG | Intron | SCLC-A |
| chr7: 2525182 | ENST00000402506.5 | LFNG | Intron | SCLC-A |
| chr7: 2525202 | ENST00000402506.5 | LFNG | Intron | SCLC-A |
| chr7: 2525203 | ENST00000402506.5 | LFNG | Intron | SCLC-A |
| chr7: 2525223 | ENST00000493850.1 | LFNG | Exon | SCLC-A |
| chr7: 2525265 | ENST00000493850.1 | LFNG | Exon | SCLC-A |
| chr7: 2525284 | ENST00000493850.1 | LFNG | Exon | SCLC-A |
| chr7: 2525297 | ENST00000493850.1 | LFNG | Exon | SCLC-A |
| chr7: 2525311 | ENST00000493850.1 | LFNG | Exon | SCLC-A |
| chr7: 2525548 | ENST00000493850.1 | LFNG | Exon | SCLC-A |
| chr7: 2525556 | ENST00000493850.1 | LFNG | Exon | SCLC-A |
| chr7: 2525597 | ENST00000402506.5 | LFNG | Intron | SCLC-A |
| chr7: 2525617 | ENST00000402506.5 | LFNG | Intron | SCLC-A |
| chr7: 2525649 | ENST00000402506.5 | LFNG | Intron | SCLC-A |
| chr7: 2525650 | ENST00000402506.5 | LFNG | Intron | SCLC-A |
| chr7: 2525685 | ENST00000402506.5 | LFNG | Exon | SCLC-A |
| chr7: 2525710 | ENST00000402506.5 | LFNG | Exon | SCLC-A |
| chr7: 2525711 | ENST00000402506.5 | LFNG | Exon | SCLC-A |
| chr7: 2525714 | ENST00000402506.5 | LFNG | Exon | SCLC-A |
| chr7: 2525715 | ENST00000402506.5 | LFNG | Exon | SCLC-A |
| chr7: 2525717 | ENST00000402506.5 | LFNG | Exon | SCLC-A |
| chr7: 2525718 | ENST00000402506.5 | LFNG | Exon | SCLC-A |
| chr7: 2525736 | ENST00000402506.5 | LFNG | Exon | SCLC-A |
| chr7: 44150961 | ENST00000673284.1 | GCK | Exon | SCLC-A |
| chr7: 44150970 | ENST00000673284.1 | GCK | Exon | SCLC-A |
| chr7: 44151070 | ENST00000673284.1 | GCK | Exon | SCLC-A |
| chr7: 44151088 | ENST00000437084.1 | GCK | Intron | SCLC-A |
| chr7: 44151089 | ENST00000437084.1 | GCK | Intron | SCLC-A |
| chr7: 44151119 | ENST00000437084.1 | GCK | Intron | SCLC-A |
| chr7: 44151120 | ENST00000437084.1 | GCK | Intron | SCLC-A |
| chr7: 44151141 | ENST00000437084.1 | GCK | Intron | SCLC-A |
| chr7: 44151294 | ENST00000437084.1 | GCK | Intron | SCLC-A |
| chr7: 44151331 | ENST00000437084.1 | GCK | Intron | SCLC-A |
| chr7: 50793906 | ENST00000644769.1 | GRB10 | Promoter | SCLC-A |
| chr7: 50793910 | ENST00000644769.1 | GRB10 | Promoter | SCLC-A |
| chr7: 50793930 | ENST00000644769.1 | GRB10 | Promoter | SCLC-A |
| chr7: 50793941 | ENST00000644769.1 | GRB10 | Promoter | SCLC-A |
| chr7: 50793958 | ENST00000644769.1 | GRB10 | Promoter | SCLC-A |
| chr7: 66147068 | ENST00000435524.2 | NA | Intron | SCLC-A |
| chr7: 66147069 | ENST00000435524.2 | NA | Intron | SCLC-A |
| chr7: 66147070 | ENST00000435524.2 | NA | Intron | SCLC-A |
| chr7: 93922083 | ENST00000248564.6 | GNG11 | Exon | SCLC-A |
| chr7: 93922100 | ENST00000248564.6 | GNG11 | Exon | SCLC-A |
| chr7: 93922205 | ENST00000248564.6 | GNG11 | Exon | SCLC-A |
| chr7: 95597100 | ENST00000665332.1 | NA | Intron | SCLC-A |
| chr7: 100493657 | ENST00000300179.7 | NYAP1 | Exon | SCLC-A |
| chr7: 100493663 | ENST00000300179.7 | NYAP1 | Exon | SCLC-A |
| chr7: 100494045 | ENST00000300179.7 | NYAP1 | Exon | SCLC-A |
| chr7: 100583557 | ENST00000310300.11 | LRCH4 | Intron | SCLC-A |
| chr7: 128940692 | ENST00000652525.1 | IRF5 | Intron | SCLC-A |
| chr7: 149879452 | ENST00000464939.1 | ATP6V0E2-AS1 | Intron | SCLC-A |
| chr7: 149879540 | ENST00000464939.1 | ATP6V0E2-AS1 | Intron | SCLC-A |
| chr7: 149879687 | ENST00000464939.1 | ATP6V0E2-AS1 | Intron | SCLC-A |
| chr7: 151090666 | ENST00000463381.5 | AGAP3 | Intron | SCLC-A |
| chr7: 151090681 | ENST00000463381.5 | AGAP3 | Intron | SCLC-A |
| chr7: 151090682 | ENST00000463381.5 | AGAP3 | Intron | SCLC-A |
| chr7: 151090719 | ENST00000463381.5 | AGAP3 | Intron | SCLC-A |
| chr7: 151090725 | ENST00000463381.5 | AGAP3 | Intron | SCLC-A |
| chr7: 151090726 | ENST00000463381.5 | AGAP3 | Intron | SCLC-A |
| chr7: 157501293 | ENST00000449903.1 | NA | Promoter | SCLC-A |
| chr7: 157501364 | ENST00000669906.1 | NA | Promoter | SCLC-A |
| chr7: 157542990 | ENST00000389413.7 | PTPRN2 | Intron | SCLC-A |
| chr7: 157566762 | ENST00000389413.7 | PTPRN2 | Intron | SCLC-A |
| chr7: 157615909 | ENST00000389413.7 | PTPRN2 | Intron | SCLC-A |
| chr7: 157678656 | ENST00000389413.7 | PTPRN2 | Intron | SCLC-A |
| chr8: 28703207 | ENST00000523149.5 | EXTL3 | Intron | SCLC-A |
| chr8: 41835983 | ENST00000265709.13 | ANK1 | Intron | SCLC-A |
| chr8: 41835994 | ENST00000265709.13 | ANK1 | Intron | SCLC-A |
| chr8: 80301230 | ENST00000656157.1 | NA | Intron | SCLC-A |
| chr8: 80301265 | ENST00000656157.1 | NA | Intron | SCLC-A |
| chr8: 80301272 | ENST00000656157.1 | NA | Intron | SCLC-A |
| chr8: 80301325 | ENST00000656157.1 | NA | Intron | SCLC-A |
| chr8: 80301357 | ENST00000656157.1 | NA | Intron | SCLC-A |
| chr8: 80301489 | ENST00000656157.1 | NA | Intron | SCLC-A |
| chr8: 81631134 | ENST00000522318.1 | IMPA1P1 | Exon | SCLC-A |
| chr8: 81631136 | ENST00000522318.1 | IMPA1P1 | Promoter | SCLC-A |
| chr8: 81631189 | ENST00000522318.1 | IMPA1P1 | Promoter | SCLC-A |
| chr8: 81631196 | ENST00000522318.1 | IMPA1P1 | Promoter | SCLC-A |
| chr8: 134499858 | ENST00000429442.6 | ZFAT | Intron | SCLC-A |
| chr8: 138855526 | ENST00000303045.11 | COL22A1 | Intron | SCLC-A |
| chr8: 139604660 | ENST00000650269.1 | KCNK9 | Intron | SCLC-A |
| chr8: 139674334 | ENST00000522317.5 | KCNK9 | Intron | SCLC-A |
| chr8: 142254143 | ENST00000524325.6 | TSNARE1 | Intron | SCLC-A |
| chr8: 142254185 | ENST00000524325.6 | TSNARE1 | Intron | SCLC-A |
| chr8: 142254280 | ENST00000524325.6 | TSNARE1 | Intron | SCLC-A |
| chr8: 142254302 | ENST00000524325.6 | TSNARE1 | Intron | SCLC-A |
| chr8: 144810882 | ENST00000525105.5 | ZNF517 | Exon | SCLC-A |
| chr8: 144810898 | ENST00000525105.5 | ZNF517 | Exon | SCLC-A |
| chr8: 144810916 | ENST00000525105.5 | ZNF517 | Exon | SCLC-A |
| chr8: 144810920 | ENST00000525105.5 | ZNF517 | Exon | SCLC-A |
| chr9: 93299724 | ENST00000411624.5 | WNK2 | Intron | SCLC-A |
| chr9: 93299816 | ENST00000411624.5 | WNK2 | Intron | SCLC-A |
| chr9: 93299821 | ENST00000411624.5 | WNK2 | Intron | SCLC-A |
| chr9: 93299844 | ENST00000411624.5 | WNK2 | Intron | SCLC-A |
| chr9: 114347854 | ENST00000307564.8 | AKNA | Exon | SCLC-A |
| chr9: 123390810 | ENST00000373624.6 | DENND1A | Intron | SCLC-A |
| chr9: 123390838 | ENST00000373624.6 | DENND1A | Intron | SCLC-A |
| chr9: 129495781 | ENST00000653047.1 | LINC00963 | Intron | SCLC-A |
| chr9: 131579248 | ENST00000372189.7 | RAPGEF1 | Exon | SCLC-A |
| chr9: 131579298 | ENST00000372189.7 | RAPGEF1 | Exon | SCLC-A |
| chr9: 131579299 | ENST00000372189.7 | RAPGEF1 | Exon | SCLC-A |
| chr9: 131598232 | ENST00000372189.7 | RAPGEF1 | Exon | SCLC-A |
| chr9: 131598281 | ENST00000372189.7 | RAPGEF1 | Exon | SCLC-A |
| chr9: 131598349 | ENST00000372189.7 | RAPGEF1 | Intron | SCLC-A |
| chr9: 136231817 | ENST00000358701.9 | QSOX2 | Intron | SCLC-A |
| chr9: 136231853 | ENST00000358701.9 | QSOX2 | Intron | SCLC-A |
| chr9: 136326076 | ENST00000573103.2 | DKFZP434A062 | Exon | SCLC-A |
| chr9: 136326115 | ENST00000573103.2 | DKFZP434A062 | Exon | SCLC-A |
| chr9: 136326179 | ENST00000573103.2 | DKFZP434A062 | Exon | SCLC-A |
| chr9: 136326197 | ENST00000573103.2 | DKFZP434A062 | Exon | SCLC-A |
| chr9: 136326251 | ENST00000573103.2 | DKFZP434A062 | Exon | SCLC-A |
| chr9: 136326252 | ENST00000573103.2 | DKFZP434A062 | Exon | SCLC-A |
| chr9: 136326273 | ENST00000573103.2 | DKFZP434A062 | Exon | SCLC-A |
| chr9: 136326274 | ENST00000573103.2 | DKFZP434A062 | Exon | SCLC-A |
| chr9: 136326278 | ENST00000573103.2 | DKFZP434A062 | Exon | SCLC-A |
| chr9: 136326279 | ENST00000573103.2 | DKFZP434A062 | Exon | SCLC-A |
| chr9: 137877782 | ENST00000371390.1 | LOC100133077 | Intron | SCLC-A |

TABLE 21
Additional methylation sites associated with the SCLC-N subtype

| Methylation Site | Transcript_ID | Gene_Symbol | Gene_Region | Subtype |
|---|---|---|---|---|
| chr1: 1348040 | ENST00000378891.9 | DVL1 | Intron | SCLC-N |
| chr1: 2318110 | ENST00000661319.1 | NA | Intron | SCLC-N |
| chr1: 3526032 | ENST00000356575.9 | MEGF6 | Intron | SCLC-N |
| chr1: 5917369 | ENST00000378169.7 | NPHP4 | Intron | SCLC-N |
| chr1: 5917443 | ENST00000378169.7 | NPHP4 | Intron | SCLC-N |
| chr1: 5917479 | ENST00000378169.7 | NPHP4 | Intron | SCLC-N |
| chr1: 6095341 | ENST00000652911.1 | KCNAB2 | Exon | SCLC-N |
| chr1: 6378302 | ENST00000608083.5 | ACOT7 | Intron | SCLC-N |
| chr1: 6378308 | ENST00000608083.5 | ACOT7 | Intron | SCLC-N |
| chr1: 6907148 | ENST00000303635.12 | CAMTA1 | Intron | SCLC-N |
| chr1: 7374098 | ENST00000303635.12 | CAMTA1 | Intron | SCLC-N |
| chr1: 7455576 | ENST00000303635.12 | CAMTA1 | Intron | SCLC-N |
| chr1: 7455653 | ENST00000303635.12 | CAMTA1 | Intron | SCLC-N |
| chr1: 7455816 | ENST00000303635.12 | CAMTA1 | Intron | SCLC-N |
| chr1: 7455821 | ENST00000303635.12 | CAMTA1 | Intron | SCLC-N |
| chr1: 8681153 | ENST00000656437.1 | RERE | Intron | SCLC-N |
| chr1: 9688039 | ENST00000377346.9 | PIK3CD | Intron | SCLC-N |
| chr1: 10658362 | ENST00000377022.8 | CASZ1 | Intron | SCLC-N |
| chr1: 10658432 | ENST00000377022.8 | CASZ1 | Intron | SCLC-N |
| chr1: 10658482 | ENST00000377022.8 | CASZ1 | Intron | SCLC-N |
| chr1: 10658483 | ENST00000377022.8 | CASZ1 | Intron | SCLC-N |
| chr1: 10658549 | ENST00000492173.1 | CASZ1 | Exon | SCLC-N |
| chr1: 10658573 | ENST00000492173.1 | CASZ1 | Exon | SCLC-N |
| chr1: 10658604 | ENST00000377022.8 | CASZ1 | Intron | SCLC-N |
| chr1: 10658877 | ENST00000377022.8 | CASZ1 | Intron | SCLC-N |
| chr1: 10704628 | ENST00000377022.8 | CASZ1 | Intron | SCLC-N |
| chr1: 10948360 | ENST00000476357.1 | C1orf127 | Exon | SCLC-N |
| chr1: 10948392 | ENST00000476357.1 | C1orf127 | Exon | SCLC-N |
| chr1: 10948648 | ENST00000476357.1 | C1orf127 | Exon | SCLC-N |
| chr1: 10948809 | ENST00000476357.1 | C1orf127 | Exon | SCLC-N |
| chr1: 15735083 | ENST00000453804.1 | NA | Intron | SCLC-N |
| chr1: 15765675 | ENST00000502739.5 | FBLIM1 | Intron | SCLC-N |
| chr1: 15765717 | ENST00000502739.5 | FBLIM1 | Intron | SCLC-N |
| chr1: 18482237 | ENST00000400664.2 | KLHDC7A | Exon | SCLC-N |
| chr1: 18482266 | ENST00000400664.2 | KLHDC7A | Exon | SCLC-N |
| chr1: 18482380 | ENST00000400664.2 | KLHDC7A | Exon | SCLC-N |
| chr1: 21428308 | ENST00000453025.1 | NA | Promoter | SCLC-N |
| chr1: 21428311 | ENST00000453025.1 | NA | Promoter | SCLC-N |
| chr1: 25771501 | ENST00000611903.4 | MAN1C1 | Intron | SCLC-N |
| chr1: 25771542 | ENST00000611903.4 | MAN1C1 | Intron | SCLC-N |
| chr1: 25771803 | ENST00000496532.5 | MAN1C1 | Promoter | SCLC-N |
| chr1: 26162435 | ENST00000374268.5 | FAM110D | Exon | SCLC-N |
| chr1: 26162459 | ENST00000374268.5 | FAM110D | Exon | SCLC-N |
| chr1: 28869066 | ENST00000234961.7 | OPRD1 | Exon | SCLC-N |
| chr1: 28869195 | ENST00000234961.7 | OPRD1 | Exon | SCLC-N |
| chr1: 28869207 | ENST00000234961.7 | OPRD1 | Exon | SCLC-N |
| chr1: 28869208 | ENST00000234961.7 | OPRD1 | Exon | SCLC-N |
| chr1: 28869229 | ENST00000234961.7 | OPRD1 | Exon | SCLC-N |
| chr1: 28869230 | ENST00000234961.7 | OPRD1 | Exon | SCLC-N |
| chr1: 28869236 | ENST00000234961.7 | OPRD1 | Exon | SCLC-N |
| chr1: 28869237 | ENST00000234961.7 | OPRD1 | Exon | SCLC-N |
| chr1: 28869354 | ENST00000234961.7 | OPRD1 | Exon | SCLC-N |
| chr1: 28869382 | ENST00000234961.7 | OPRD1 | Exon | SCLC-N |
| chr1: 28869383 | ENST00000234961.7 | OPRD1 | Exon | SCLC-N |
| chr1: 31626915 | ENST00000373705.1 | HCRTR1 | Intron | SCLC-N |
| chr1: 31626982 | ENST00000373705.1 | HCRTR1 | Intron | SCLC-N |
| chr1: 42735278 | ENST00000539749.5 | CLDN19 | Exon | SCLC-N |
| chr1: 42735300 | ENST00000539749.5 | CLDN19 | Exon | SCLC-N |
| chr1: 42735392 | ENST00000539749.5 | CLDN19 | Exon | SCLC-N |
| chr1: 42735571 | ENST00000539749.5 | CLDN19 | Exon | SCLC-N |
| chr1: 44637731 | ENST00000355387.6 | RNF220 | Intron | SCLC-N |
| chr1: 46285986 | ENST00000498402.2 | LRRC41 | Exon | SCLC-N |
| chr1: 46285995 | ENST00000498402.2 | LRRC41 | Exon | SCLC-N |
| chr1: 50421427 | ENST00000418121.5 | DMRTA2 | Exon | SCLC-N |
| chr1: 50421430 | ENST00000418121.5 | DMRTA2 | Exon | SCLC-N |
| chr1: 50421439 | ENST00000418121.5 | DMRTA2 | Exon | SCLC-N |
| chr1: 50421445 | ENST00000418121.5 | DMRTA2 | Exon | SCLC-N |
| chr1: 50421447 | ENST00000418121.5 | DMRTA2 | Exon | SCLC-N |
| chr1: 50421454 | ENST00000418121.5 | DMRTA2 | Exon | SCLC-N |
| chr1: 50421466 | ENST00000418121.5 | DMRTA2 | Exon | SCLC-N |
| chr1: 50421472 | ENST00000418121.5 | DMRTA2 | Exon | SCLC-N |
| chr1: 50421475 | ENST00000418121.5 | DMRTA2 | Exon | SCLC-N |
| chr1: 50421483 | ENST00000418121.5 | DMRTA2 | Exon | SCLC-N |
| chr1: 50421484 | ENST00000418121.5 | DMRTA2 | Exon | SCLC-N |
| chr1: 50421489 | ENST00000418121.5 | DMRTA2 | Exon | SCLC-N |
| chr1: 50421490 | ENST00000418121.5 | DMRTA2 | Exon | SCLC-N |
| chr1: 50421501 | ENST00000418121.5 | DMRTA2 | Exon | SCLC-N |
| chr1: 50421504 | ENST00000418121.5 | DMRTA2 | Exon | SCLC-N |
| chr1: 55063332 | ENST00000302118.5 | PCSK9 | Intron | SCLC-N |
| chr1: 55063374 | ENST00000302118.5 | PCSK9 | Exon | SCLC-N |
| chr1: 55063384 | ENST00000302118.5 | PCSK9 | Exon | SCLC-N |
| chr1: 55063449 | ENST00000302118.5 | PCSK9 | Exon | SCLC-N |
| chr1: 60897215 | ENST00000371191.5 | NFIA | Intron | SCLC-N |
| chr1: 61052398 | ENST00000371191.5 | NFIA | Intron | SCLC-N |
| chr1: 88682776 | ENST00000458097.5 | PKN2-AS1 | Intron | SCLC-N |
| chr1: 90841513 | ENST00000669273.1 | LINC02609 | Intron | SCLC-N |
| chr1: 112871178 | ENST00000658577.1 | NA | Intron | SCLC-N |
| chr1: 115655113 | ENST00000355485.7 | VANGL1 | Intron | SCLC-N |
| chr1: 154842667 | ENST00000271915.9 | KCNN3 | Intron | SCLC-N |
| chr1: 159011154 | ENST00000566111.5 | IFI16 | Intron | SCLC-N |
| chr1: 161198356 | ENST00000479948.5 | NDUFS2 | Intron | SCLC-N |
| chr1: 161198378 | ENST00000479948.5 | NDUFS2 | Intron | SCLC-N |
| chr1: 161198400 | ENST00000479948.5 | NDUFS2 | Intron | SCLC-N |
| chr1: 161198403 | ENST00000479948.5 | NDUFS2 | Intron | SCLC-N |
| chr1: 161198404 | ENST00000479948.5 | NDUFS2 | Intron | SCLC-N |
| chr1: 162373995 | ENST00000431696.1 | NOS1AP | Intron | SCLC-N |
| chr1: 180174185 | ENST00000367602.8 | QSOX1 | Intron | SCLC-N |
| chr1: 180182136 | ENST00000367602.8 | QSOX1 | Intron | SCLC-N |
| chr1: 180182208 | ENST00000367602.8 | QSOX1 | Exon | SCLC-N |
| chr1: 180182213 | ENST00000367602.8 | QSOX1 | Exon | SCLC-N |
| chr1: 180182264 | ENST00000367602.8 | QSOX1 | Exon | SCLC-N |
| chr1: 180182292 | ENST00000367602.8 | QSOX1 | Exon | SCLC-N |
| chr1: 180183034 | ENST00000367602.8 | QSOX1 | Intron | SCLC-N |
| chr1: 180183061 | ENST00000367602.8 | QSOX1 | Intron | SCLC-N |
| chr1: 180183067 | ENST00000367602.8 | QSOX1 | Intron | SCLC-N |
| chr1: 180183068 | ENST00000367602.8 | QSOX1 | Intron | SCLC-N |
| chr1: 180183222 | ENST00000367602.8 | QSOX1 | Intron | SCLC-N |
| chr1: 180460155 | ENST00000642319.1 | LHX4-AS1 | Intron | SCLC-N |
| chr1: 180941072 | ENST00000461346.1 | KIAA1614 | Promoter | SCLC-N |
| chr1: 201942012 | ENST00000367288.5 | LMOD1 | Intron | SCLC-N |
| chr1: 204447512 | ENST00000367187.7 | PIK3C2B | Exon | SCLC-N |
| chr1: 204481681 | ENST00000415899.1 | PIK3C2B | Intron | SCLC-N |
| chr1: 204481806 | ENST00000415899.1 | PIK3C2B | Intron | SCLC-N |
| chr1: 205064748 | ENST00000636641.2 | CNTN2 | Promoter | SCLC-N |
| chr1: 205519116 | ENST00000507067.1 | CDK18 | Intron | SCLC-N |
| chr1: 206490930 | ENST00000581977.7 | IKBKE | Intron | SCLC-N |
| chr1: 206702548 | ENST00000294981.8 | MAPKAPK2 | Intron | SCLC-N |
| chr1: 207848368 | ENST00000637970.1 | MIR29B2CHG | Intron | SCLC-N |
| chr1: 207848391 | ENST00000637970.1 | MIR29B2CHG | Intron | SCLC-N |
| chr1: 207848493 | ENST00000637970.1 | MIR29B2CHG | Intron | SCLC-N |
| chr1: 208120134 | ENST00000367033.4 | PLXNA2 | Intron | SCLC-N |
| chr1: 208231439 | ENST00000367033.4 | PLXNA2 | Intron | SCLC-N |
| chr1: 212916596 | ENST00000632866.1 | NA | Promoter | SCLC-N |
| chr1: 212981590 | ENST00000366966.6 | VASH2 | Intron | SCLC-N |
| chr1: 212982124 | ENST00000366966.6 | VASH2 | Intron | SCLC-N |
| chr1: 212982320 | ENST00000366966.6 | VASH2 | Intron | SCLC-N |
| chr1: 214551221 | ENST00000366956.10 | PTPN14 | UTR5 | SCLC-N |
| chr1: 226540981 | ENST00000671364.1 | NA | Intron | SCLC-N |
| chr1: 226589203 | ENST00000366788.8 | STUM | Intron | SCLC-N |
| chr1: 226589217 | ENST00000366788.8 | STUM | Intron | SCLC-N |
| chr1: 231693853 | ENST00000602873.5 | DISC1 | Intron | SCLC-N |
| chr1: 231693854 | ENST00000602873.5 | DISC1 | Intron | SCLC-N |
| chr1: 231693861 | ENST00000602873.5 | DISC1 | Intron | SCLC-N |
| chr1: 231693867 | ENST00000602873.5 | DISC1 | Intron | SCLC-N |
| chr1: 231693870 | ENST00000602873.5 | DISC1 | Intron | SCLC-N |
| chr1: 231693879 | ENST00000602873.5 | DISC1 | Intron | SCLC-N |
| chr1: 231693880 | ENST00000602873.5 | DISC1 | Intron | SCLC-N |
| chr1: 231693886 | ENST00000602873.5 | DISC1 | Intron | SCLC-N |
| chr1: 231693887 | ENST00000602873.5 | DISC1 | Intron | SCLC-N |
| chr1: 231693921 | ENST00000602873.5 | DISC1 | Intron | SCLC-N |
| chr1: 231693994 | ENST00000602873.5 | DISC1 | Intron | SCLC-N |
| chr1: 231694044 | ENST00000602873.5 | DISC1 | Intron | SCLC-N |
| chr1: 231694399 | ENST00000602873.5 | DISC1 | Intron | SCLC-N |
| chr1: 231694419 | ENST00000602873.5 | DISC1 | Intron | SCLC-N |
| chr1: 234235867 | ENST00000366618.8 | SLC35F3 | Intron | SCLC-N |
| chr1: 234235881 | ENST00000366618.8 | SLC35F3 | Intron | SCLC-N |
| chr1: 245212635 | ENST00000407071.7 | KIF26B | Intron | SCLC-N |
| chr10: 1650866 | ENST00000381312.6 | ADARB2 | Intron | SCLC-N |
| chr10: 1650867 | ENST00000381312.6 | ADARB2 | Intron | SCLC-N |
| chr10: 1650882 | ENST00000381312.6 | ADARB2 | Intron | SCLC-N |
| chr10: 1650883 | ENST00000381312.6 | ADARB2 | Intron | SCLC-N |
| chr10: 3781749 | ENST00000173785.4 | KLF6 | Intron | SCLC-N |
| chr10: 3781785 | ENST00000173785.4 | KLF6 | Intron | SCLC-N |
| chr10: 3782363 | ENST00000173785.4 | KLF6 | Promoter | SCLC-N |
| chr10: 3782364 | ENST00000173785.4 | KLF6 | Promoter | SCLC-N |
| chr10: 3782374 | ENST00000173785.4 | KLF6 | Promoter | SCLC-N |
| chr10: 23960136 | ENST00000376462.5 | KIAA1217 | Intron | SCLC-N |
| chr10: 23960137 | ENST00000376462.5 | KIAA1217 | Intron | SCLC-N |
| chr10: 23960206 | ENST00000376462.5 | KIAA1217 | Intron | SCLC-N |
| chr10: 24300973 | ENST00000376462.5 | KIAA1217 | Intron | SCLC-N |
| chr10: 51123578 | ENST00000401604.8 | PRKG1 | Intron | SCLC-N |
| chr10: 51123611 | ENST00000401604.8 | PRKG1 | Intron | SCLC-N |
| chr10: 69374479 | ENST00000436817.6 | HK1 | Intron | SCLC-N |
| chr10: 69375951 | ENST00000436817.6 | HK1 | Intron | SCLC-N |
| chr10: 74134133 | ENST00000355264.8 | AP3M1 | Exon | SCLC-N |
| chr10: 99879892 | ENST00000543621.6 | DNMBP | Exon | SCLC-N |
| chr10: 102294627 | ENST00000369983.3 | GBF1 | Intron | SCLC-N |
| chr10: 102294628 | ENST00000369983.3 | GBF1 | Intron | SCLC-N |
| chr10: 112952176 | ENST00000534894.5 | TCF7L2 | Intron | SCLC-N |
| chr10: 116275192 | ENST00000648277.1 | NA | Exon | SCLC-N |
| chr10: 119282537 | ENST00000392870.3 | GRK5 | Intron | SCLC-N |
| chr10: 119282564 | ENST00000392870.3 | GRK5 | Intron | SCLC-N |
| chr10: 119282662 | ENST00000392870.3 | GRK5 | Intron | SCLC-N |
| chr10: 121580588 | ENST00000356226.8 | FGFR2 | Intron | SCLC-N |
| chr10: 121580759 | ENST00000356226.8 | FGFR2 | Intron | SCLC-N |
| chr10: 124478693 | ENST00000368842.10 | LHPP | Intron | SCLC-N |
| chr10: 124478697 | ENST00000368842.10 | LHPP | Intron | SCLC-N |
| chr10: 124478718 | ENST00000368842.10 | LHPP | Intron | SCLC-N |
| chr10: 127736157 | ENST00000623953.1 | NA | Exon | SCLC-N |
| chr10: 127736181 | ENST00000623953.1 | NA | Exon | SCLC-N |
| chr10: 132811036 | ENST00000368586.10 | CFAP46 | Intron | SCLC-N |
| chr10: 132811041 | ENST00000368586.10 | CFAP46 | Intron | SCLC-N |
| chr11: 888359 | ENST00000526714.5 | CHID1 | Intron | SCLC-N |
| chr11: 888460 | ENST00000526714.5 | CHID1 | Intron | SCLC-N |
| chr11: 888461 | ENST00000526714.5 | CHID1 | Intron | SCLC-N |
| chr11: 888482 | ENST00000526714.5 | CHID1 | Intron | SCLC-N |
| chr11: 888519 | ENST00000526714.5 | CHID1 | Intron | SCLC-N |
| chr11: 888547 | ENST00000526714.5 | CHID1 | Intron | SCLC-N |
| chr11: 888548 | ENST00000526714.5 | CHID1 | Intron | SCLC-N |
| chr11: 888556 | ENST00000526714.5 | CHID1 | Intron | SCLC-N |
| chr11: 888557 | ENST00000526714.5 | CHID1 | Intron | SCLC-N |
| chr11: 888561 | ENST00000526714.5 | CHID1 | Intron | SCLC-N |
| chr11: 888562 | ENST00000526714.5 | CHID1 | Intron | SCLC-N |
| chr11: 888597 | ENST00000526714.5 | CHID1 | Intron | SCLC-N |
| chr11: 1450652 | ENST00000544817.5 | BRSK2 | Promoter | SCLC-N |
| chr11: 1450687 | ENST00000308219.13 | BRSK2 | Exon | SCLC-N |
| chr11: 1746746 | ENST00000636579.1 | IFITM10 | Intron | SCLC-N |
| chr11: 2718090 | ENST00000155840.11 | KCNQ1 | Intron | SCLC-N |
| chr11: 17449179 | ENST00000645744.1 | ABCC8 | Intron | SCLC-N |
| chr11: 17449216 | ENST00000645744.1 | ABCC8 | Intron | SCLC-N |
| chr11: 17449222 | ENST00000645744.1 | ABCC8 | Intron | SCLC-N |
| chr11: 19832413 | ENST00000360655.8 | NAV2 | Intron | SCLC-N |
| chr11: 19832533 | ENST00000360655.8 | NAV2 | Exon | SCLC-N |
| chr11: 22338192 | ENST00000263160.4 | SLC17A6 | Promoter | SCLC-N |
| chr11: 22338261 | ENST00000263160.4 | SLC17A6 | Promoter | SCLC-N |
| chr11: 31804635 | ENST00000638965.1 | PAX6 | Intron | SCLC-N |
| chr11: 31804694 | ENST00000638965.1 | PAX6 | Intron | SCLC-N |
| chr11: 31805187 | ENST00000638965.1 | PAX6 | Intron | SCLC-N |
| chr11: 31805192 | ENST00000638965.1 | PAX6 | Intron | SCLC-N |
| chr11: 31805210 | ENST00000638965.1 | PAX6 | Intron | SCLC-N |
| chr11: 31805286 | ENST00000638965.1 | PAX6 | Intron | SCLC-N |
| chr11: 31805292 | ENST00000638965.1 | PAX6 | Intron | SCLC-N |
| chr11: 31805302 | ENST00000638965.1 | PAX6 | Intron | SCLC-N |
| chr11: 31805419 | ENST00000638965.1 | PAX6 | Intron | SCLC-N |
| chr11: 31805474 | ENST00000638965.1 | PAX6 | Intron | SCLC-N |
| chr11: 31805500 | ENST00000638965.1 | PAX6 | Intron | SCLC-N |
| chr11: 31820660 | ENST00000530348.5 | RCN1 | Intron | SCLC-N |
| chr11: 32166325 | ENST00000419556.2 | NA | Intron | SCLC-N |
| chr11: 32311336 | ENST00000525689.2 | NA | Intron | SCLC-N |
| chr11: 34502672 | ENST00000429939.6 | ELF5 | Intron | SCLC-N |
| chr11: 59762173 | ENST00000641815.1 | STX3 | Intron | SCLC-N |
| chr11: 59762256 | ENST00000641815.1 | STX3 | Intron | SCLC-N |
| chr11: 60411065 | ENST00000531787.5 | MS4A14 | Intron | SCLC-N |
| chr11: 60411092 | ENST00000531787.5 | MS4A14 | Intron | SCLC-N |
| chr11: 60411097 | ENST00000531787.5 | MS4A14 | Intron | SCLC-N |
| chr11: 60524906 | ENST00000527948.1 | MS4A13 | Intron | SCLC-N |
| chr11: 60524907 | ENST00000527948.1 | MS4A13 | Intron | SCLC-N |
| chr11: 60524912 | ENST00000527948.1 | MS4A13 | Intron | SCLC-N |
| chr11: 61696210 | ENST00000257215.10 | DAGLA | Intron | SCLC-N |
| chr11: 61696297 | ENST00000257215.10 | DAGLA | Intron | SCLC-N |
| chr11: 61696393 | ENST00000257215.10 | DAGLA | Intron | SCLC-N |
| chr11: 72584678 | ENST00000441209.6 | PDE2A | Promoter | SCLC-N |
| chr11: 72584886 | ENST00000441209.6 | PDE2A | Promoter | SCLC-N |
| chr11: 72584892 | ENST00000441209.6 | PDE2A | Promoter | SCLC-N |
| chr11: 72584895 | ENST00000441209.6 | PDE2A | Promoter | SCLC-N |
| chr11: 121111283 | ENST00000645041.1 | TBCEL | Intron | SCLC-N |
| chr11: 126165892 | ENST00000532357.1 | NA | Intron | SCLC-N |
| chr11: 126165940 | ENST00000532357.1 | NA | Intron | SCLC-N |
| chr11: 126165948 | ENST00000532357.1 | NA | Intron | SCLC-N |
| chr11: 126165949 | ENST00000532357.1 | NA | Intron | SCLC-N |
| chr11: 126356903 | ENST00000227495.10 | ST3GAL4 | Intron | SCLC-N |
| chr11: 126585523 | ENST00000548204.1 | NA | Intron | SCLC-N |
| chr11: 126585530 | ENST00000548204.1 | NA | Intron | SCLC-N |
| chr11: 126749417 | ENST00000525144.7 | KIRREL3 | Intron | SCLC-N |
| chr11: 126749456 | ENST00000525144.7 | KIRREL3 | Intron | SCLC-N |
| chr11: 128913895 | ENST00000529694.6 | KCNJ5 | Intron | SCLC-N |
| chr12: 1500501 | ENST00000515614.3 | LINC00942 | Promoter | SCLC-N |
| chr12: 1500510 | ENST00000515614.3 | LINC00942 | Promoter | SCLC-N |
| chr12: 1500511 | ENST00000515614.3 | LINC00942 | Promoter | SCLC-N |
| chr12: 1822018 | ENST00000444595.6 | CACNA2D4 | Intron | SCLC-N |
| chr12: 6633693 | ENST00000329858.9 | LPAR5 | Intron | SCLC-N |
| chr12: 30988264 | ENST00000546076.5 | TSPAN11 | Intron | SCLC-N |
| chr12: 42479754 | ENST00000639958.1 | PRICKLE1 | Intron | SCLC-N |
| chr12: 42497409 | ENST00000639958.1 | PRICKLE1 | Intron | SCLC-N |
| chr12: 42497410 | ENST00000639958.1 | PRICKLE1 | Intron | SCLC-N |
| chr12: 51377533 | ENST00000543196.6 | GALNT6 | Intron | SCLC-N |
| chr12: 57225138 | ENST00000349394.6 | NXPH4 | Exon | SCLC-N |
| chr12: 77047970 | ENST00000416496.6 | E2F7 | Intron | SCLC-N |
| chr12: 96233859 | ENST00000552142.5 | ELK3 | Intron | SCLC-N |
| chr12: 96234054 | ENST00000552142.5 | ELK3 | Intron | SCLC-N |
| chr12: 97465331 | ENST00000538559.6 | RMST | Intron | SCLC-N |
| chr12: 97596152 | ENST00000639121.1 | RMST | Intron | SCLC-N |
| chr12: 108804526 | ENST00000546433.5 | SSH1 | Intron | SCLC-N |
| chr12: 112946864 | ENST00000552784.1 | NA | Intron | SCLC-N |
| chr12: 112946932 | ENST00000552784.1 | NA | Intron | SCLC-N |
| chr12: 112946963 | ENST00000552784.1 | NA | Intron | SCLC-N |
| chr12: 122355274 | ENST00000541410.1 | CLIP1 | Exon | SCLC-N |
| chr12: 124463768 | ENST00000448008.1 | NCOR2 | Intron | SCLC-N |
| chr12: 130463309 | ENST00000261655.8 | RIMBP2 | Intron | SCLC-N |
| chr12: 130463326 | ENST00000261655.8 | RIMBP2 | Intron | SCLC-N |
| chr13: 98214193 | ENST00000595437.5 | FARP1 | Intron | SCLC-N |
| chr13: 109495155 | ENST00000650264.1 | NA | Intron | SCLC-N |
| chr13: 111598392 | ENST00000375713.1 | NA | Intron | SCLC-N |
| chr13: 111598399 | ENST00000375713.1 | NA | Intron | SCLC-N |
| chr14: 24158185 | ENST00000362934.1 | NA | Promoter | SCLC-N |
| chr14: 74946474 | ENST00000555253.1 | PGF | Exon | SCLC-N |
| chr14: 74946478 | ENST00000555253.1 | PGF | Exon | SCLC-N |
| chr14: 76396996 | ENST00000512784.6 | ESRRB | Intron | SCLC-N |
| chr14: 76397003 | ENST00000512784.6 | ESRRB | Intron | SCLC-N |
| chr14: 89284792 | ENST00000345097.8 | FOXN3 | Intron | SCLC-N |
| chr14: 91170655 | ENST00000518649.5 | DGLUCY | Intron | SCLC-N |
| chr14: 91375767 | ENST00000389857.11 | CCDC88C | Intron | SCLC-N |
| chr14: 93737728 | ENST00000393143.5 | PRIMA1 | Intron | SCLC-N |
| chr14: 93752608 | ENST00000393143.5 | PRIMA1 | Intron | SCLC-N |
| chr14: 94001245 | ENST00000444118.5 | CCDC197 | Exon | SCLC-N |
| chr14: 102630887 | ENST00000262241.7 | RCOR1 | Intron | SCLC-N |
| chr14: 103104365 | ENST00000560925.5 | EXOC3L4 | Exon | SCLC-N |
| chr14: 103104368 | ENST00000560925.5 | EXOC3L4 | Exon | SCLC-N |
| chr14: 105173746 | ENST00000548203.1 | JAG2 | Intron | SCLC-N |
| chr15: 28886855 | ENST00000561069.5 | APBA2 | Intron | SCLC-N |
| chr15: 34580510 | ENST00000569100.5 | GOLGA8B | Intron | SCLC-N |
| chr15: 34580589 | ENST00000569100.5 | GOLGA8B | Intron | SCLC-N |
| chr15: 40291333 | ENST00000559671.5 | PLCB2 | Promoter | SCLC-N |
| chr15: 40291340 | ENST00000559671.5 | PLCB2 | Promoter | SCLC-N |
| chr15: 40291349 | ENST00000559671.5 | PLCB2 | Promoter | SCLC-N |
| chr15: 40291352 | ENST00000559671.5 | PLCB2 | Promoter | SCLC-N |
| chr15: 40291354 | ENST00000559671.5 | PLCB2 | Promoter | SCLC-N |
| chr15: 45187264 | ENST00000560034.1 | NA | Intron | SCLC-N |
| chr15: 45187265 | ENST00000560034.1 | NA | Intron | SCLC-N |
| chr15: 45187278 | ENST00000560034.1 | NA | Intron | SCLC-N |
| chr15: 45187279 | ENST00000560034.1 | NA | Intron | SCLC-N |
| chr15: 45187312 | ENST00000560034.1 | NA | Intron | SCLC-N |
| chr15: 58065916 | ENST00000558239.5 | ALDH1A2 | Intron | SCLC-N |
| chr15: 65271021 | ENST00000444347.2 | PARP16 | Intron | SCLC-N |
| chr15: 67829883 | ENST00000341418.10 | SKOR1 | Intron | SCLC-N |
| chr15: 67829885 | ENST00000341418.10 | SKOR1 | Intron | SCLC-N |
| chr15: 67829889 | ENST00000341418.10 | SKOR1 | Intron | SCLC-N |
| chr15: 67829891 | ENST00000341418.10 | SKOR1 | Intron | SCLC-N |
| chr15: 67829908 | ENST00000341418.10 | SKOR1 | Intron | SCLC-N |
| chr15: 67829910 | ENST00000341418.10 | SKOR1 | Intron | SCLC-N |
| chr15: 67829912 | ENST00000341418.10 | SKOR1 | Intron | SCLC-N |
| chr15: 74826515 | ENST00000564823.1 | CPLX3 | Exon | SCLC-N |
| chr15: 74826550 | ENST00000564823.1 | CPLX3 | Exon | SCLC-N |
| chr15: 74826559 | ENST00000564823.1 | CPLX3 | Exon | SCLC-N |
| chr15: 74826560 | ENST00000564823.1 | CPLX3 | Exon | SCLC-N |
| chr15: 74826565 | ENST00000564823.1 | CPLX3 | Exon | SCLC-N |
| chr15: 74826566 | ENST00000564823.1 | CPLX3 | Exon | SCLC-N |
| chr15: 74826569 | ENST00000564823.1 | CPLX3 | Exon | SCLC-N |
| chr15: 74826570 | ENST00000564823.1 | CPLX3 | Exon | SCLC-N |
| chr15: 74826572 | ENST00000564823.1 | CPLX3 | Exon | SCLC-N |
| chr15: 74826573 | ENST00000564823.1 | CPLX3 | Exon | SCLC-N |
| chr15: 74826577 | ENST00000564823.1 | CPLX3 | Exon | SCLC-N |
| chr15: 74826586 | ENST00000564823.1 | CPLX3 | Exon | SCLC-N |
| chr15: 74826587 | ENST00000564823.1 | CPLX3 | Exon | SCLC-N |
| chr15: 74826593 | ENST00000564823.1 | CPLX3 | Exon | SCLC-N |
| chr15: 74826594 | ENST00000564823.1 | CPLX3 | Exon | SCLC-N |
| chr15: 74826595 | ENST00000564823.1 | CPLX3 | Exon | SCLC-N |
| chr15: 74826596 | ENST00000564823.1 | CPLX3 | Exon | SCLC-N |
| chr15: 74826601 | ENST00000564823.1 | CPLX3 | Exon | SCLC-N |
| chr15: 74826617 | ENST00000564823.1 | CPLX3 | Exon | SCLC-N |
| chr15: 74826618 | ENST00000564823.1 | CPLX3 | Exon | SCLC-N |
| chr15: 74826619 | ENST00000564823.1 | CPLX3 | Exon | SCLC-N |
| chr15: 74826620 | ENST00000564823.1 | CPLX3 | Exon | SCLC-N |
| chr15: 74826642 | ENST00000564823.1 | CPLX3 | Exon | SCLC-N |
| chr15: 74826693 | ENST00000564823.1 | CPLX3 | Exon | SCLC-N |
| chr15: 74826696 | ENST00000564823.1 | CPLX3 | Exon | SCLC-N |
| chr15: 74826697 | ENST00000564823.1 | CPLX3 | Exon | SCLC-N |
| chr15: 74826708 | ENST00000564823.1 | CPLX3 | Exon | SCLC-N |
| chr15: 80445702 | ENST00000529181.1 | ARNT2 | Intron | SCLC-N |
| chr15: 89815305 | ENST00000300060.7 | ANPEP | Promoter | SCLC-N |
| chr15: 92819642 | ENST00000653163.1 | CHASERR | Exon | SCLC-N |
| chr15: 96321706 | ENST00000502125.6 | NR2F2-AS1 | Intron | SCLC-N |
| chr15: 98915596 | ENST00000560144.1 | IGF1R | Intron | SCLC-N |
| chr15: 98915756 | ENST00000560144.1 | IGF1R | Intron | SCLC-N |
| chr16: 629397 | ENST00000573440.1 | NA | Exon | SCLC-N |
| chr16: 629476 | ENST00000573440.1 | NA | Exon | SCLC-N |
| chr16: 629563 | ENST00000573440.1 | NA | Exon | SCLC-N |
| chr16: 629635 | ENST00000573440.1 | NA | Exon | SCLC-N |
| chr16: 629671 | ENST00000573440.1 | NA | Exon | SCLC-N |
| chr16: 629785 | ENST00000573440.1 | NA | Exon | SCLC-N |
| chr16: 629797 | ENST00000573440.1 | NA | Exon | SCLC-N |
| chr16: 1158781 | ENST00000638323.1 | CACNA1H | Intron | SCLC-N |
| chr16: 1158792 | ENST00000638323.1 | CACNA1H | Intron | SCLC-N |
| chr16: 1158995 | ENST00000638323.1 | CACNA1H | Intron | SCLC-N |
| chr16: 2283037 | ENST00000569062.1 | ABCA3 | Intron | SCLC-N |
| chr16: 2283209 | ENST00000569062.1 | ABCA3 | Exon | SCLC-N |
| chr16: 11349827 | ENST00000649869.1 | NA | Intron | SCLC-N |
| chr16: 11349915 | ENST00000649869.1 | NA | Intron | SCLC-N |
| chr16: 11609643 | ENST00000574848.5 | LITAF | Intron | SCLC-N |
| chr16: 16067230 | ENST00000399408.6 | ABCC1 | Intron | SCLC-N |
| chr16: 16067239 | ENST00000399408.6 | ABCC1 | Intron | SCLC-N |
| chr16: 16067303 | ENST00000399408.6 | ABCC1 | Intron | SCLC-N |
| chr16: 16067337 | ENST00000399408.6 | ABCC1 | Intron | SCLC-N |
| chr16: 16067364 | ENST00000399408.6 | ABCC1 | Intron | SCLC-N |
| chr16: 17972619 | ENST00000567304.1 | NA | Intron | SCLC-N |
| chr16: 49491744 | ENST00000561648.5 | ZNF423 | Intron | SCLC-N |
| chr16: 49499397 | ENST00000561648.5 | ZNF423 | Intron | SCLC-N |
| chr16: 55324765 | ENST00000558730.2 | NA | Intron | SCLC-N |
| chr16: 55324781 | ENST00000558730.2 | NA | Intron | SCLC-N |
| chr16: 64947764 | ENST00000394156.7 | CDH11 | Exon | SCLC-N |
| chr16: 64947795 | ENST00000394156.7 | CDH11 | Exon | SCLC-N |
| chr16: 64947825 | ENST00000394156.7 | CDH11 | Exon | SCLC-N |
| chr16: 64950797 | ENST00000394156.7 | CDH11 | Exon | SCLC-N |
| chr16: 64950806 | ENST00000394156.7 | CDH11 | Exon | SCLC-N |
| chr16: 69819451 | ENST00000359154.6 | WWP2 | Intron | SCLC-N |
| chr16: 85287074 | ENST00000637419.1 | GSE1 | Intron | SCLC-N |
| chr16: 85287098 | ENST00000637419.1 | GSE1 | Intron | SCLC-N |
| chr16: 85287146 | ENST00000637419.1 | GSE1 | Intron | SCLC-N |
| chr16: 85287168 | ENST00000637419.1 | GSE1 | Intron | SCLC-N |
| chr16: 87315250 | ENST00000618367.4 | C16orf95 | Intron | SCLC-N |
| chr16: 87315257 | ENST00000618367.4 | C16orf95 | Intron | SCLC-N |
| chr16: 88886878 | ENST00000565053.1 | LOC101927793 | Exon | SCLC-N |
| chr16: 88886885 | ENST00000565053.1 | LOC101927793 | Exon | SCLC-N |
| chr16: 88886907 | ENST00000565053.1 | LOC101927793 | Exon | SCLC-N |
| chr16: 88886921 | ENST00000565053.1 | LOC101927793 | Exon | SCLC-N |
| chr16: 88886923 | ENST00000565053.1 | LOC101927793 | Exon | SCLC-N |
| chr16: 88886926 | ENST00000565053.1 | LOC101927793 | Exon | SCLC-N |
| chr16: 88886927 | ENST00000565053.1 | LOC101927793 | Exon | SCLC-N |
| chr16: 88886933 | ENST00000565053.1 | LOC101927793 | Exon | SCLC-N |
| chr17: 7384103 | ENST00000577009.1 | TNK1 | Promoter | SCLC-N |
| chr17: 7384126 | ENST00000577009.1 | TNK1 | Promoter | SCLC-N |
| chr17: 7384133 | ENST00000577009.1 | TNK1 | Promoter | SCLC-N |
| chr17: 7384155 | ENST00000577009.1 | TNK1 | Promoter | SCLC-N |
| chr17: 9212847 | ENST00000173229.7 | NTN1 | Intron | SCLC-N |
| chr17: 17814663 | ENST00000485080.6 | SREBF1 | Promoter | SCLC-N |
| chr17: 17814785 | ENST00000385104.1 | MIR33B | Promoter | SCLC-N |
| chr17: 17814792 | ENST00000385104.1 | MIR33B | Promoter | SCLC-N |
| chr17: 17814825 | ENST00000385104.1 | MIR33B | Promoter | SCLC-N |
| chr17: 17815211 | ENST00000478616.1 | SREBF1 | Promoter | SCLC-N |
| chr17: 17815297 | ENST00000478616.1 | SREBF1 | Promoter | SCLC-N |
| chr17: 18992147 | ENST00000417251.6 | SLC5A10 | Intron | SCLC-N |
| chr17: 21453364 | ENST00000654263.1 | NA | Intron | SCLC-N |
| chr17: 32520284 | ENST00000318217.10 | MYO1D | Intron | SCLC-N |
| chr17: 37507464 | ENST00000617516.5 | DUSP14 | Intron | SCLC-N |
| chr17: 37507514 | ENST00000617516.5 | DUSP14 | Intron | SCLC-N |
| chr17: 37512702 | ENST00000614411.1 | DUSP14 | Exon | SCLC-N |
| chr17: 37512725 | ENST00000614411.1 | DUSP14 | Exon | SCLC-N |
| chr17: 37512756 | ENST00000614411.1 | DUSP14 | Exon | SCLC-N |
| chr17: 37512886 | ENST00000614411.1 | DUSP14 | Exon | SCLC-N |
| chr17: 37512900 | ENST00000614411.1 | DUSP14 | Exon | SCLC-N |
| chr17: 37512901 | ENST00000614411.1 | DUSP14 | Exon | SCLC-N |
| chr17: 37512907 | ENST00000614411.1 | DUSP14 | Exon | SCLC-N |
| chr17: 48543208 | ENST00000504972.3 | HOXB-AS1 | Promoter | SCLC-N |
| chr17: 50278974 | ENST00000511882.1 | TMEM92 | Exon | SCLC-N |
| chr17: 50469322 | ENST00000510410.5 | ACSF2 | Intron | SCLC-N |
| chr17: 50469335 | ENST00000510410.5 | ACSF2 | Intron | SCLC-N |
| chr17: 50469412 | ENST00000510410.5 | ACSF2 | Intron | SCLC-N |
| chr17: 50469415 | ENST00000510410.5 | ACSF2 | Intron | SCLC-N |
| chr17: 50469437 | ENST00000510410.5 | ACSF2 | Intron | SCLC-N |
| chr17: 50469491 | ENST00000510410.5 | ACSF2 | Intron | SCLC-N |
| chr17: 57863248 | ENST00000577830.6 | CUEDC1 | Exon | SCLC-N |
| chr17: 58863486 | ENST00000308249.4 | PPM1E | Intron | SCLC-N |
| chr17: 67652568 | ENST00000581322.6 | PITPNC1 | Intron | SCLC-N |
| chr17: 67652633 | ENST00000581322.6 | PITPNC1 | Intron | SCLC-N |
| chr17: 67652635 | ENST00000581322.6 | PITPNC1 | Intron | SCLC-N |
| chr17: 68450868 | ENST00000590353.1 | PRKAR1A | Intron | SCLC-N |
| chr17: 68450875 | ENST00000590353.1 | PRKAR1A | Intron | SCLC-N |
| chr17: 68450884 | ENST00000590353.1 | PRKAR1A | Intron | SCLC-N |
| chr17: 72122815 | ENST00000628742.2 | SOX9-AS1 | Intron | SCLC-N |
| chr17: 72122840 | ENST00000628742.2 | SOX9-AS1 | Intron | SCLC-N |
| chr17: 72122875 | ENST00000628742.2 | SOX9-AS1 | Intron | SCLC-N |
| chr17: 74341931 | ENST00000551294.5 | KIF19 | Exon | SCLC-N |
| chr17: 74341937 | ENST00000551294.5 | KIF19 | Exon | SCLC-N |
| chr17: 78384877 | ENST00000589425.5 | PGS1 | Intron | SCLC-N |
| chr17: 79465820 | ENST00000583458.5 | RBFOX3 | Intron | SCLC-N |
| chr17: 79465841 | ENST00000583458.5 | RBFOX3 | Intron | SCLC-N |
| chr17: 81171168 | ENST00000414089.5 | PVALEF | Intron | SCLC-N |
| chr17: 81171184 | ENST00000414089.5 | PVALEF | Intron | SCLC-N |
| chr17: 81171304 | ENST00000414089.5 | PVALEF | Intron | SCLC-N |
| chr18: 5890423 | ENST00000581347.2 | TMEM200C | Exon | SCLC-N |
| chr18: 5890435 | ENST00000581347.2 | TMEM200C | Exon | SCLC-N |
| chr18: 5891082 | ENST00000581347.2 | TMEM200C | Exon | SCLC-N |
| chr18: 5891091 | ENST00000581347.2 | TMEM200C | Exon | SCLC-N |
| chr18: 49799492 | ENST00000285093.15 | ACAA2 | Intron | SCLC-N |
| chr18: 49799496 | ENST00000285093.15 | ACAA2 | Intron | SCLC-N |
| chr18: 50721810 | ENST00000592595.5 | MAPK4 | Intron | SCLC-N |
| chr18: 50721992 | ENST00000592595.5 | MAPK4 | Intron | SCLC-N |
| chr18: 80040054 | ENST00000569722.5 | RBFA | Intron | SCLC-N |
| chr18: 80040058 | ENST00000569722.5 | RBFA | Intron | SCLC-N |
| chr19: 427246 | ENST00000590170.3 | SHC2 | Intron | SCLC-N |
| chr19: 1071308 | ENST00000591293.2 | ARHGAP45 | Intron | SCLC-N |
| chr19: 1071314 | ENST00000591293.2 | ARHGAP45 | Intron | SCLC-N |
| chr19: 1071455 | ENST00000591293.2 | ARHGAP45 | Intron | SCLC-N |
| chr19: 3180037 | ENST00000246115.5 | S1PR4 | Exon | SCLC-N |
| chr19: 3196028 | ENST00000588428.5 | NCLN | Intron | SCLC-N |
| chr19: 3196045 | ENST00000588428.5 | NCLN | Intron | SCLC-N |
| chr19: 3196296 | ENST00000590671.5 | NCLN | Intron | SCLC-N |
| chr19: 3196350 | ENST00000590671.5 | NCLN | Intron | SCLC-N |
| chr19: 3196353 | ENST00000590671.5 | NCLN | Intron | SCLC-N |
| chr19: 3196354 | ENST00000590671.5 | NCLN | Intron | SCLC-N |
| chr19: 3196369 | ENST00000590671.5 | NCLN | Intron | SCLC-N |
| chr19: 3196370 | ENST00000590671.5 | NCLN | Intron | SCLC-N |
| chr19: 3196390 | ENST00000590671.5 | NCLN | Intron | SCLC-N |
| chr19: 3196394 | ENST00000590671.5 | NCLN | Intron | SCLC-N |
| chr19: 3196395 | ENST00000590671.5 | NCLN | Intron | SCLC-N |
| chr19: 3196430 | ENST00000590671.5 | NCLN | Intron | SCLC-N |
| chr19: 3444440 | ENST00000641145.1 | NFIC | Intron | SCLC-N |
| chr19: 5048951 | ENST00000611640.4 | KDM4B | Intron | SCLC-N |
| chr19: 6074048 | ENST00000589742.5 | RFX2 | Intron | SCLC-N |
| chr19: 14434616 | ENST00000585619.5 | PKN1 | Intron | SCLC-N |
| chr19: 15452847 | ENST00000599694.1 | RASAL3 | Intron | SCLC-N |
| chr19: 15452855 | ENST00000599694.1 | RASAL3 | Intron | SCLC-N |
| chr19: 15452871 | ENST00000599694.1 | RASAL3 | Intron | SCLC-N |
| chr19: 15453399 | ENST00000609274.5 | RASAL3 | Exon | SCLC-N |
| chr19: 15453406 | ENST00000609274.5 | RASAL3 | Exon | SCLC-N |
| chr19: 18369135 | ENST00000597663.2 | PGPEP1 | Intron | SCLC-N |
| chr19: 18489610 | ENST00000262809.9 | ELL | Intron | SCLC-N |
| chr19: 18743299 | ENST00000321949.13 | CRTC1 | Intron | SCLC-N |
| chr19: 18743305 | ENST00000321949.13 | CRTC1 | Intron | SCLC-N |
| chr19: 18743312 | ENST00000321949.13 | CRTC1 | Intron | SCLC-N |
| chr19: 18743357 | ENST00000321949.13 | CRTC1 | Intron | SCLC-N |
| chr19: 18743708 | ENST00000321949.13 | CRTC1 | Intron | SCLC-N |
| chr19: 18743753 | ENST00000321949.13 | CRTC1 | Intron | SCLC-N |
| chr19: 18743756 | ENST00000321949.13 | CRTC1 | Intron | SCLC-N |
| chr19: 18743757 | ENST00000321949.13 | CRTC1 | Intron | SCLC-N |
| chr19: 18743807 | ENST00000321949.13 | CRTC1 | Intron | SCLC-N |
| chr19: 18744990 | ENST00000321949.13 | CRTC1 | Intron | SCLC-N |
| chr19: 18745096 | ENST00000321949.13 | CRTC1 | Intron | SCLC-N |
| chr19: 18745109 | ENST00000321949.13 | CRTC1 | Intron | SCLC-N |
| chr19: 18745203 | ENST00000321949.13 | CRTC1 | Intron | SCLC-N |
| chr19: 32971317 | ENST00000305768.10 | CEP89 | Intron | SCLC-N |
| chr19: 35033769 | ENST00000415950.4 | SCN1B | Exon | SCLC-N |
| chr19: 35033778 | ENST00000415950.4 | SCN1B | Exon | SCLC-N |
| chr19: 35033779 | ENST00000415950.4 | SCN1B | Exon | SCLC-N |
| chr19: 36111275 | ENST00000633214.1 | OVOL3 | Exon | SCLC-N |
| chr19: 36111643 | ENST00000633214.1 | OVOL3 | Intron | SCLC-N |
| chr19: 38252905 | ENST00000590510.5 | SPINT2 | Intron | SCLC-N |
| chr19: 38253105 | ENST00000590510.5 | SPINT2 | Intron | SCLC-N |
| chr19: 40692091 | ENST00000593724.1 | COQ8B | Exon | SCLC-N |
| chr19: 40692102 | ENST00000593724.1 | COQ8B | Exon | SCLC-N |
| chr19: 40692117 | ENST00000593724.1 | COQ8B | Exon | SCLC-N |
| chr19: 40807102 | ENST00000601627.1 | CYP2G1P | Intron | SCLC-N |
| chr19: 40807147 | ENST00000601627.1 | CYP2G1P | Intron | SCLC-N |
| chr19: 49104726 | ENST00000544278.2 | SNRNP70 | Exon | SCLC-N |
| chr19: 49104729 | ENST00000544278.2 | SNRNP70 | Exon | SCLC-N |
| chr19: 49432507 | ENST00000221485.8 | SLC17A7 | Intron | SCLC-N |
| chr19: 49432522 | ENST00000221485.8 | SLC17A7 | Exon | SCLC-N |
| chr19: 50543096 | ENST00000599957.5 | LRRC4B | Intron | SCLC-N |
| chr19: 55359687 | ENST00000585734.5 | FAM71E2 | Exon | SCLC-N |
| chr2: 1946826 | ENST00000650081.1 | MYT1L | Intron | SCLC-N |
| chr2: 1946869 | ENST00000650081.1 | MYT1L | Intron | SCLC-N |
| chr2: 1947005 | ENST00000650081.1 | MYT1L | Intron | SCLC-N |
| chr2: 7020543 | ENST00000467276.5 | RNF144A | Exon | SCLC-N |
| chr2: 9379013 | ENST00000315273.4 | ASAP2 | Exon | SCLC-N |
| chr2: 9379072 | ENST00000315273.4 | ASAP2 | Intron | SCLC-N |
| chr2: 10163597 | ENST00000381786.7 | NA | Intron | SCLC-N |
| chr2: 10163607 | ENST00000381786.7 | NA | Intron | SCLC-N |
| chr2: 10342807 | ENST00000423674.5 | HPCAL1 | Intron | SCLC-N |
| chr2: 23621054 | ENST00000486442.6 | KLHL29 | Intron | SCLC-N |
| chr2: 25419491 | ENST00000496972.6 | DTNB | Intron | SCLC-N |
| chr2: 25419728 | ENST00000496972.6 | DTNB | Intron | SCLC-N |
| chr2: 67561523 | ENST00000433065.1 | NA | Promoter | SCLC-N |
| chr2: 72921047 | ENST00000394111.6 | EMX1 | Intron | SCLC-N |
| chr2: 72921051 | ENST00000394111.6 | EMX1 | Intron | SCLC-N |
| chr2: 74647996 | ENST00000536235.5 | M1AP | Intron | SCLC-N |
| chr2: 74648089 | ENST00000536235.5 | M1AP | Intron | SCLC-N |
| chr2: 74648095 | ENST00000536235.5 | M1AP | Intron | SCLC-N |
| chr2: 74648101 | ENST00000536235.5 | M1AP | Intron | SCLC-N |
| chr2: 74648113 | ENST00000536235.5 | M1AP | Intron | SCLC-N |
| chr2: 74648116 | ENST00000536235.5 | M1AP | Intron | SCLC-N |
| chr2: 74648129 | ENST00000536235.5 | M1AP | Intron | SCLC-N |
| chr2: 74648130 | ENST00000536235.5 | M1AP | Intron | SCLC-N |
| chr2: 74648136 | ENST00000536235.5 | M1AP | Intron | SCLC-N |
| chr2: 74648141 | ENST00000536235.5 | M1AP | Intron | SCLC-N |
| chr2: 74648148 | ENST00000536235.5 | M1AP | Intron | SCLC-N |
| chr2: 74648177 | ENST00000536235.5 | M1AP | Intron | SCLC-N |
| chr2: 74648178 | ENST00000536235.5 | M1AP | Intron | SCLC-N |
| chr2: 74648184 | ENST00000536235.5 | M1AP | Intron | SCLC-N |
| chr2: 74648191 | ENST00000536235.5 | M1AP | Intron | SCLC-N |
| chr2: 74648207 | ENST00000536235.5 | M1AP | Intron | SCLC-N |
| chr2: 74648217 | ENST00000536235.5 | M1AP | Intron | SCLC-N |
| chr2: 74648218 | ENST00000536235.5 | M1AP | Intron | SCLC-N |
| chr2: 74648227 | ENST00000536235.5 | M1AP | Intron | SCLC-N |
| chr2: 74648245 | ENST00000536235.5 | M1AP | Intron | SCLC-N |
| chr2: 74648250 | ENST00000536235.5 | M1AP | Intron | SCLC-N |
| chr2: 74648260 | ENST00000536235.5 | M1AP | Intron | SCLC-N |
| chr2: 74648261 | ENST00000536235.5 | M1AP | Intron | SCLC-N |
| chr2: 74648273 | ENST00000290536.9 | M1AP | Promoter | SCLC-N |
| chr2: 74648280 | ENST00000290536.9 | M1AP | Promoter | SCLC-N |
| chr2: 85243788 | ENST00000282111.4 | TCF7L1 | Intron | SCLC-N |
| chr2: 109498113 | ENST00000309415.8 | SH3RF3 | Intron | SCLC-N |
| chr2: 110807569 | ENST00000389811.8 | ACOXL | Intron | SCLC-N |
| chr2: 110807672 | ENST00000389811.8 | ACOXL | Intron | SCLC-N |
| chr2: 110807894 | ENST00000389811.8 | ACOXL | Intron | SCLC-N |
| chr2: 110807930 | ENST00000389811.8 | ACOXL | Intron | SCLC-N |
| chr2: 110807936 | ENST00000389811.8 | ACOXL | Intron | SCLC-N |
| chr2: 144404074 | ENST00000638087.1 | ZEB2 | Exon | SCLC-N |
| chr2: 181656540 | ENST00000479558.5 | CERKL | Intron | SCLC-N |
| chr2: 181656605 | ENST00000479558.5 | CERKL | Intron | SCLC-N |
| chr2: 233060554 | ENST00000359570.9 | INPP5D | Exon | SCLC-N |
| chr2: 233060566 | ENST00000359570.9 | INPP5D | Exon | SCLC-N |
| chr2: 233060578 | ENST00000359570.9 | INPP5D | Exon | SCLC-N |
| chr2: 233060591 | ENST00000359570.9 | INPP5D | Exon | SCLC-N |
| chr2: 237509061 | ENST00000422695.5 | MLPH | Intron | SCLC-N |
| chr2: 237509070 | ENST00000422695.5 | MLPH | Intron | SCLC-N |
| chr20: 11247428 | ENST00000444792.1 | LOC339593 | Intron | SCLC-N |
| chr20: 20498264 | ENST00000202677.12 | RALGAPA2 | Intron | SCLC-N |
| chr20: 20498374 | ENST00000202677.12 | RALGAPA2 | Intron | SCLC-N |
| chr20: 21101285 | ENST00000655093.1 | LINC00237 | Intron | SCLC-N |
| chr20: 21101299 | ENST00000655093.1 | LINC00237 | Intron | SCLC-N |
| chr20: 21101362 | ENST00000655093.1 | LINC00237 | Intron | SCLC-N |
| chr20: 21101370 | ENST00000655093.1 | LINC00237 | Intron | SCLC-N |
| chr20: 21101484 | ENST00000655093.1 | LINC00237 | Intron | SCLC-N |
| chr20: 21101554 | ENST00000655093.1 | LINC00237 | Intron | SCLC-N |
| chr20: 23085065 | ENST00000246006.5 | CD93 | Exon | SCLC-N |
| chr20: 33675075 | ENST00000606690.5 | NECAB3 | Promoter | SCLC-N |
| chr20: 33675097 | ENST00000606690.5 | NECAB3 | Promoter | SCLC-N |
| chr20: 36528428 | ENST00000439595.5 | DLGAP4-AS1 | Intron | SCLC-N |
| chr20: 36529592 | ENST00000439595.5 | DLGAP4-AS1 | Intron | SCLC-N |
| chr20: 36852420 | ENST00000237536.9 | SOGA1 | Intron | SCLC-N |
| chr20: 36852432 | ENST00000237536.9 | SOGA1 | Intron | SCLC-N |
| chr20: 53592723 | ENST00000431687.5 | NA | Intron | SCLC-N |
| chr20: 56629325 | ENST00000416606.1 | TFAP2C | Promoter | SCLC-N |
| chr21: 26572915 | ENST00000652641.2 | CYYR1 | Exon | SCLC-N |
| chr21: 26573110 | ENST00000652641.2 | CYYR1 | Exon | SCLC-N |
| chr21: 26573118 | ENST00000652641.2 | CYYR1 | Exon | SCLC-N |
| chr21: 26573124 | ENST00000652641.2 | CYYR1 | Exon | SCLC-N |
| chr21: 26573132 | ENST00000652641.2 | CYYR1 | Exon | SCLC-N |
| chr21: 26573134 | ENST00000652641.2 | CYYR1 | Exon | SCLC-N |
| chr21: 26573168 | ENST00000652641.2 | CYYR1 | Exon | SCLC-N |
| chr21: 26573169 | ENST00000652641.2 | CYYR1 | Exon | SCLC-N |
| chr21: 26573204 | ENST00000652641.2 | CYYR1 | Exon | SCLC-N |
| chr21: 44521445 | ENST00000323084.9 | TSPEAR | Intron | SCLC-N |
| chr21: 44646833 | ENST00000323084.9 | TSPEAR | Intron | SCLC-N |
| chr22: 23678260 | ENST00000651432.1 | GUSBP11 | Intron | SCLC-N |
| chr22: 23678347 | ENST00000651432.1 | GUSBP11 | Intron | SCLC-N |
| chr22: 50603643 | ENST00000329492.6 | MAPK8IP2 | Exon | SCLC-N |
| chr3: 50365613 | ENST00000606589.1 | CYB561D2 | Intron | SCLC-N |
| chr3: 50365664 | ENST00000606589.1 | CYB561D2 | Intron | SCLC-N |
| chr3: 50365696 | ENST00000606589.1 | CYB561D2 | Intron | SCLC-N |
| chr3: 50365700 | ENST00000606589.1 | CYB561D2 | Intron | SCLC-N |
| chr3: 50365707 | ENST00000606589.1 | CYB561D2 | Intron | SCLC-N |
| chr3: 61796965 | ENST00000474889.6 | PTPRG | Intron | SCLC-N |
| chr3: 97949951 | ENST00000506682.5 | RIOX2 | Exon | SCLC-N |
| chr3: 97949984 | ENST00000506682.5 | RIOX2 | Exon | SCLC-N |
| chr3: 109192259 | ENST00000648541.1 | LINC00488 | Intron | SCLC-N |
| chr3: 109192266 | ENST00000648541.1 | LINC00488 | Intron | SCLC-N |
| chr3: 109192267 | ENST00000648541.1 | LINC00488 | Intron | SCLC-N |
| chr3: 120284769 | ENST00000464295.5 | GPR156 | Intron | SCLC-N |
| chr3: 183179196 | ENST00000328913.7 | MCF2L2 | Exon | SCLC-N |
| chr3: 183179370 | ENST00000328913.7 | MCF2L2 | Exon | SCLC-N |
| chr3: 183179405 | ENST00000328913.7 | MCF2L2 | Exon | SCLC-N |
| chr3: 183179434 | ENST00000328913.7 | MCF2L2 | Exon | SCLC-N |
| chr3: 183179443 | ENST00000328913.7 | MCF2L2 | Exon | SCLC-N |
| chr3: 186099220 | ENST00000472868.6 | ETV5 | Intron | SCLC-N |
| chr3: 194305594 | ENST00000654752.1 | LINC00887 | Intron | SCLC-N |
| chr4: 6957533 | ENST00000448507.5 | TBC1D14 | Intron | SCLC-N |
| chr4: 6969656 | ENST00000448507.5 | TBC1D14 | Intron | SCLC-N |
| chr4: 6969657 | ENST00000448507.5 | TBC1D14 | Intron | SCLC-N |
| chr4: 6969701 | ENST00000448507.5 | TBC1D14 | Intron | SCLC-N |
| chr4: 6969706 | ENST00000448507.5 | TBC1D14 | Intron | SCLC-N |
| chr4: 10587871 | ENST00000226951.11 | CLNK | Intron | SCLC-N |
| chr4: 10587872 | ENST00000226951.11 | CLNK | Intron | SCLC-N |
| chr4: 10587881 | ENST00000226951.11 | CLNK | Intron | SCLC-N |
| chr4: 10587882 | ENST00000226951.11 | CLNK | Intron | SCLC-N |
| chr4: 52751099 | ENST00000650700.1 | RASL11B | Intron | SCLC-N |
| chr5: 1857480 | ENST00000509455.1 | LINC02116 | Promoter | SCLC-N |
| chr5: 66359037 | ENST00000663200.1 | NA | Intron | SCLC-N |
| chr5: 93588643 | ENST00000327111.8 | NR2F1 | Intron | SCLC-N |
| chr5: 93588648 | ENST00000327111.8 | NR2F1 | Intron | SCLC-N |
| chr5: 141430928 | ENST00000517417.2 | PCDHGA1 | Intron | SCLC-N |
| chr5: 141430936 | ENST00000517417.2 | PCDHGA1 | Intron | SCLC-N |
| chr5: 141430938 | ENST00000517417.2 | PCDHGA1 | Intron | SCLC-N |
| chr5: 141430943 | ENST00000517417.2 | PCDHGA1 | Intron | SCLC-N |
| chr5: 141492421 | ENST00000517417.2 | PCDHGA1 | Intron | SCLC-N |
| chr5: 172244750 | ENST00000393792.3 | UBTD2 | Intron | SCLC-N |
| chr5: 172244889 | ENST00000393792.3 | UBTD2 | Intron | SCLC-N |
| chr6: 25318583 | ENST00000643807.1 | CMAHP | Intron | SCLC-N |
| chr6: 25318616 | ENST00000643807.1 | CMAHP | Intron | SCLC-N |
| chr6: 35469210 | ENST00000467020.5 | RPL10A | Exon | SCLC-N |
| chr6: 35469377 | ENST00000467020.5 | RPL10A | Exon | SCLC-N |
| chr6: 136923394 | ENST00000331858.5 | SLC35D3 | Intron | SCLC-N |
| chr7: 1933538 | ENST00000265854.11 | MAD1L1 | Intron | SCLC-N |
| chr7: 1933549 | ENST00000265854.11 | MAD1L1 | Intron | SCLC-N |
| chr7: 1933588 | ENST00000265854.11 | MAD1L1 | Intron | SCLC-N |
| chr7: 44151126 | ENST00000437084.1 | GCK | Intron | SCLC-N |
| chr7: 44151127 | ENST00000437084.1 | GCK | Intron | SCLC-N |
| chr7: 44151142 | ENST00000437084.1 | GCK | Intron | SCLC-N |
| chr7: 44151330 | ENST00000437084.1 | GCK | Intron | SCLC-N |
| chr7: 66147063 | ENST00000435524.2 | NA | Intron | SCLC-N |
| chr7: 129775659 | ENST00000362288.1 | MIR96 | Promoter | SCLC-N |
| chr7: 149879536 | ENST00000464939.1 | ATP6V0E2-AS1 | Intron | SCLC-N |
| chr7: 149879711 | ENST00000464939.1 | ATP6V0E2-AS1 | Intron | SCLC-N |
| chr7: 151115113 | ENST00000463381.5 | AGAP3 | Intron | SCLC-N |
| chr7: 151115129 | ENST00000463381.5 | AGAP3 | Intron | SCLC-N |
| chr7: 151143289 | ENST00000463381.5 | AGAP3 | Intron | SCLC-N |
| chr7: 151143354 | ENST00000463381.5 | AGAP3 | Exon | SCLC-N |
| chr7: 151143363 | ENST00000463381.5 | AGAP3 | Exon | SCLC-N |
| chr8: 80301254 | ENST00000656157.1 | NA | Intron | SCLC-N |
| chr8: 80301356 | ENST00000656157.1 | NA | Intron | SCLC-N |
| chr8: 80301477 | ENST00000656157.1 | NA | Intron | SCLC-N |
| chr8: 81631050 | ENST00000522318.1 | IMPA1P1 | Exon | SCLC-N |
| chr8: 81631185 | ENST00000522318.1 | IMPA1P1 | Promoter | SCLC-N |
| chr8: 134499786 | ENST00000429442.6 | ZFAT | Intron | SCLC-N |
| chr8: 134499855 | ENST00000429442.6 | ZFAT | Intron | SCLC-N |
| chr8: 134499859 | ENST00000429442.6 | ZFAT | Intron | SCLC-N |
| chr8: 139604536 | ENST00000650269.1 | KCNK9 | Intron | SCLC-N |
| chr8: 139604670 | ENST00000650269.1 | KCNK9 | Intron | SCLC-N |
| chr9: 93299636 | ENST00000411624.5 | WNK2 | Intron | SCLC-N |
| chr9: 93299661 | ENST00000411624.5 | WNK2 | Intron | SCLC-N |
| chr9: 93299673 | ENST00000411624.5 | WNK2 | Intron | SCLC-N |
| chr9: 93299688 | ENST00000411624.5 | WNK2 | Intron | SCLC-N |
| chr9: 113901537 | ENST00000374126.9 | ZNF618 | Intron | SCLC-N |
| chr9: 113901541 | ENST00000374126.9 | ZNF618 | Intron | SCLC-N |
| chr9: 113901566 | ENST00000374126.9 | ZNF618 | Intron | SCLC-N |
| chr9: 114347778 | ENST00000307564.8 | AKNA | Exon | SCLC-N |
| chr9: 114347782 | ENST00000307564.8 | AKNA | Exon | SCLC-N |
| chr9: 114347787 | ENST00000307564.8 | AKNA | Exon | SCLC-N |
| chr9: 114347808 | ENST00000307564.8 | AKNA | Exon | SCLC-N |
| chr9: 114347813 | ENST00000307564.8 | AKNA | Exon | SCLC-N |
| chr9: 114347860 | ENST00000307564.8 | AKNA | Exon | SCLC-N |
| chr9: 114347866 | ENST00000307564.8 | AKNA | Exon | SCLC-N |
| chr9: 123401183 | ENST00000373624.6 | DENND1A | Intron | SCLC-N |
| chr9: 123401332 | ENST00000373624.6 | DENND1A | Intron | SCLC-N |
| chr9: 136534050 | ENST00000651671.1 | NOTCH1 | Intron | SCLC-N |
| chr9: 136534055 | ENST00000651671.1 | NOTCH1 | Intron | SCLC-N |
| chr9: 136534065 | ENST00000651671.1 | NOTCH1 | Intron | SCLC-N |
| chr9: 136534074 | ENST00000651671.1 | NOTCH1 | Intron | SCLC-N |
| chr9: 137877774 | ENST00000371390.1 | LOC100133077 | Intron | SCLC-N |
| chr9: 137877776 | ENST00000371390.1 | LOC100133077 | Intron | SCLC-N |
| chr9: 137877791 | ENST00000371390.1 | LOC100133077 | Intron | SCLC-N |
| chr9: 137877794 | ENST00000371390.1 | LOC100133077 | Intron | SCLC-N |
| chr9: 137877797 | ENST00000371390.1 | LOC100133077 | Intron | SCLC-N |
| chr9: 137877803 | ENST00000371390.1 | LOC100133077 | Intron | SCLC-N |
| chr9: 137877815 | ENST00000371390.1 | LOC100133077 | Intron | SCLC-N |

TABLE 22
Additional methylation sites associated with the SCLC-P subtype

| Methylation Site | Transcript_ID | Gene_Symbol | Gene_Region | Subtype |
|---|---|---|---|---|
| chr1: 958193 | ENST00000477976.5 | NOC2L | Exon | SCLC-P |
| chr1: 958206 | ENST00000477976.5 | NOC2L | Exon | SCLC-P |
| chr1: 958225 | ENST00000477976.5 | NOC2L | Exon | SCLC-P |
| chr1: 958355 | ENST00000477976.5 | NOC2L | Exon | SCLC-P |
| chr1: 958377 | ENST00000477976.5 | NOC2L | Exon | SCLC-P |
| chr1: 958407 | ENST00000477976.5 | NOC2L | Exon | SCLC-P |
| chr1: 972487 | ENST00000379410.8 | PLEKHN1 | Intron | SCLC-P |
| chr1: 972499 | ENST00000379410.8 | PLEKHN1 | Intron | SCLC-P |
| chr1: 972500 | ENST00000379410.8 | PLEKHN1 | Intron | SCLC-P |
| chr1: 972536 | ENST00000379410.8 | PLEKHN1 | Intron | SCLC-P |
| chr1: 972537 | ENST00000379410.8 | PLEKHN1 | Intron | SCLC-P |
| chr1: 972563 | ENST00000379410.8 | PLEKHN1 | Intron | SCLC-P |
| chr1: 972564 | ENST00000379410.8 | PLEKHN1 | Intron | SCLC-P |
| chr1: 972578 | ENST00000379410.8 | PLEKHN1 | Intron | SCLC-P |
| chr1: 1049344 | ENST00000379370.7 | AGRN | Exon | SCLC-P |
| chr1: 1049569 | ENST00000379370.7 | AGRN | Exon | SCLC-P |
| chr1: 1049585 | ENST00000379370.7 | AGRN | Exon | SCLC-P |
| chr1: 1060899 | ENST00000427998.5 | LOC100288175 | Intron | SCLC-P |
| chr1: 1060915 | ENST00000427998.5 | LOC100288175 | Intron | SCLC-P |
| chr1: 1060917 | ENST00000427998.5 | LOC100288175 | Intron | SCLC-P |
| chr1: 1061051 | ENST00000427998.5 | LOC100288175 | Intron | SCLC-P |
| chr1: 1061052 | ENST00000427998.5 | LOC100288175 | Intron | SCLC-P |
| chr1: 1061060 | ENST00000427998.5 | LOC100288175 | Intron | SCLC-P |
| chr1: 1067284 | ENST00000412397.2 | LOC100288175 | Intron | SCLC-P |
| chr1: 1073300 | ENST00000453464.3 | RNF223 | Intron | SCLC-P |
| chr1: 1073322 | ENST00000453464.3 | RNF223 | Intron | SCLC-P |
| chr1: 1110693 | ENST00000379325.7 | C1orf159 | Intron | SCLC-P |
| chr1: 1110698 | ENST00000379325.7 | C1orf159 | Intron | SCLC-P |
| chr1: 1110739 | ENST00000379325.7 | C1orf159 | Intron | SCLC-P |
| chr1: 1114609 | ENST00000379325.7 | C1orf159 | Intron | SCLC-P |
| chr1: 1114751 | ENST00000379325.7 | C1orf159 | Intron | SCLC-P |
| chr1: 1114755 | ENST00000379325.7 | C1orf159 | Intron | SCLC-P |
| chr1: 1217039 | ENST00000360001.11 | SDF4 | Exon | SCLC-P |
| chr1: 1217078 | ENST00000360001.11 | SDF4 | Exon | SCLC-P |
| chr1: 1217143 | ENST00000360001.11 | SDF4 | Exon | SCLC-P |
| chr1: 1217144 | ENST00000360001.11 | SDF4 | Exon | SCLC-P |
| chr1: 1217148 | ENST00000360001.11 | SDF4 | Exon | SCLC-P |
| chr1: 1217149 | ENST00000360001.11 | SDF4 | Exon | SCLC-P |
| chr1: 1217151 | ENST00000360001.11 | SDF4 | Exon | SCLC-P |
| chr1: 1217152 | ENST00000360001.11 | SDF4 | Exon | SCLC-P |
| chr1: 1217168 | ENST00000360001.11 | SDF4 | Exon | SCLC-P |
| chr1: 1217186 | ENST00000360001.11 | SDF4 | Exon | SCLC-P |
| chr1: 1217187 | ENST00000360001.11 | SDF4 | Exon | SCLC-P |
| chr1: 1217188 | ENST00000360001.11 | SDF4 | Exon | SCLC-P |
| chr1: 1217194 | ENST00000360001.11 | SDF4 | Exon | SCLC-P |
| chr1: 1217200 | ENST00000360001.11 | SDF4 | Exon | SCLC-P |
| chr1: 1217201 | ENST00000360001.11 | SDF4 | Exon | SCLC-P |
| chr1: 1217206 | ENST00000360001.11 | SDF4 | Exon | SCLC-P |
| chr1: 1217207 | ENST00000360001.11 | SDF4 | Exon | SCLC-P |
| chr1: 1217223 | ENST00000360001.11 | SDF4 | Exon | SCLC-P |
| chr1: 1217231 | ENST00000360001.11 | SDF4 | Exon | SCLC-P |
| chr1: 1217254 | ENST00000360001.11 | SDF4 | Exon | SCLC-P |
| chr1: 1217260 | ENST00000360001.11 | SDF4 | Exon | SCLC-P |
| chr1: 1217284 | ENST00000360001.11 | SDF4 | Exon | SCLC-P |
| chr1: 1217285 | ENST00000360001.11 | SDF4 | Exon | SCLC-P |
| chr1: 1217287 | ENST00000360001.11 | SDF4 | Exon | SCLC-P |
| chr1: 1217305 | ENST00000360001.11 | SDF4 | Exon | SCLC-P |
| chr1: 1217313 | ENST00000360001.11 | SDF4 | Exon | SCLC-P |
| chr1: 1217317 | ENST00000360001.11 | SDF4 | Exon | SCLC-P |
| chr1: 1217318 | ENST00000360001.11 | SDF4 | Exon | SCLC-P |
| chr1: 1217319 | ENST00000360001.11 | SDF4 | Exon | SCLC-P |
| chr1: 1217339 | ENST00000360001.11 | SDF4 | Exon | SCLC-P |
| chr1: 1223730 | ENST00000360001.11 | SDF4 | Intron | SCLC-P |
| chr1: 1224575 | ENST00000360001.11 | SDF4 | Intron | SCLC-P |
| chr1: 1232875 | ENST00000459994.2 | SDF4 | Promoter | SCLC-P |
| chr1: 1232876 | ENST00000459994.2 | SDF4 | Promoter | SCLC-P |
| chr1: 1232881 | ENST00000459994.2 | SDF4 | Promoter | SCLC-P |
| chr1: 1232882 | ENST00000459994.2 | SDF4 | Promoter | SCLC-P |
| chr1: 1232896 | ENST00000459994.2 | SDF4 | Promoter | SCLC-P |
| chr1: 1232897 | ENST00000459994.2 | SDF4 | Promoter | SCLC-P |
| chr1: 1232914 | ENST00000459994.2 | SDF4 | Promoter | SCLC-P |
| chr1: 1232915 | ENST00000459994.2 | SDF4 | Promoter | SCLC-P |
| chr1: 1232916 | ENST00000459994.2 | SDF4 | Promoter | SCLC-P |
| chr1: 1232917 | ENST00000459994.2 | SDF4 | Promoter | SCLC-P |
| chr1: 1232923 | ENST00000459994.2 | SDF4 | Promoter | SCLC-P |
| chr1: 1232924 | ENST00000459994.2 | SDF4 | Promoter | SCLC-P |
| chr1: 1232926 | ENST00000459994.2 | SDF4 | Promoter | SCLC-P |
| chr1: 1232927 | ENST00000459994.2 | SDF4 | Promoter | SCLC-P |
| chr1: 1232932 | ENST00000459994.2 | SDF4 | Promoter | SCLC-P |
| chr1: 1232944 | ENST00000459994.2 | SDF4 | Promoter | SCLC-P |
| chr1: 1232945 | ENST00000459994.2 | SDF4 | Promoter | SCLC-P |
| chr1: 1232963 | ENST00000459994.2 | SDF4 | Promoter | SCLC-P |
| chr1: 1232964 | ENST00000459994.2 | SDF4 | Promoter | SCLC-P |
| chr1: 1232972 | ENST00000360001.11 | SDF4 | Promoter | SCLC-P |
| chr1: 1232973 | ENST00000360001.11 | SDF4 | Promoter | SCLC-P |
| chr1: 1232974 | ENST00000360001.11 | SDF4 | Promoter | SCLC-P |
| chr1: 1232975 | ENST00000360001.11 | SDF4 | Promoter | SCLC-P |
| chr1: 1232984 | ENST00000360001.11 | SDF4 | Promoter | SCLC-P |
| chr1: 1232985 | ENST00000360001.11 | SDF4 | Promoter | SCLC-P |
| chr1: 1232986 | ENST00000360001.11 | SDF4 | Promoter | SCLC-P |
| chr1: 1232987 | ENST00000360001.11 | SDF4 | Promoter | SCLC-P |
| chr1: 1232998 | ENST00000360001.11 | SDF4 | Promoter | SCLC-P |
| chr1: 1232999 | ENST00000360001.11 | SDF4 | Promoter | SCLC-P |
| chr1: 1233016 | ENST00000263741.12 | SDF4 | Promoter | SCLC-P |
| chr1: 1233017 | ENST00000263741.12 | SDF4 | Promoter | SCLC-P |
| chr1: 1233031 | ENST00000263741.12 | SDF4 | Promoter | SCLC-P |
| chr1: 1233077 | ENST00000379198.5 | B3GALT6 | Exon | SCLC-P |
| chr1: 1233149 | ENST00000379198.5 | B3GALT6 | Exon | SCLC-P |
| chr1: 1233151 | ENST00000379198.5 | B3GALT6 | Exon | SCLC-P |
| chr1: 1233154 | ENST00000379198.5 | B3GALT6 | Exon | SCLC-P |
| chr1: 1233160 | ENST00000379198.5 | B3GALT6 | Exon | SCLC-P |
| chr1: 1233162 | ENST00000379198.5 | B3GALT6 | Exon | SCLC-P |
| chr1: 1233164 | ENST00000379198.5 | B3GALT6 | Exon | SCLC-P |
| chr1: 1233198 | ENST00000379198.5 | B3GALT6 | Exon | SCLC-P |
| chr1: 1233209 | ENST00000379198.5 | B3GALT6 | Exon | SCLC-P |
| chr1: 1233224 | ENST00000379198.5 | B3GALT6 | Exon | SCLC-P |
| chr1: 1233226 | ENST00000379198.5 | B3GALT6 | Exon | SCLC-P |
| chr1: 1233229 | ENST00000379198.5 | B3GALT6 | Exon | SCLC-P |
| chr1: 1233272 | ENST00000379198.5 | B3GALT6 | Exon | SCLC-P |
| chr1: 1233275 | ENST00000379198.5 | B3GALT6 | Exon | SCLC-P |
| chr1: 1233277 | ENST00000379198.5 | B3GALT6 | Exon | SCLC-P |
| chr1: 1233282 | ENST00000379198.5 | B3GALT6 | Exon | SCLC-P |
| chr1: 1295076 | ENST00000492936.5 | ACAP3 | Intron | SCLC-P |
| chr1: 1295077 | ENST00000492936.5 | ACAP3 | Intron | SCLC-P |
| chr1: 1295086 | ENST00000492936.5 | ACAP3 | Intron | SCLC-P |
| chr1: 1347350 | ENST00000378891.9 | DVL1 | Intron | SCLC-P |
| chr1: 1347360 | ENST00000378891.9 | DVL1 | Intron | SCLC-P |
| chr1: 1374140 | ENST00000338370.7 | AURKAIP1 | Exon | SCLC-P |
| chr1: 1387723 | ENST00000480479.5 | CCNL2 | Intron | SCLC-P |
| chr1: 1387741 | ENST00000480479.5 | CCNL2 | Intron | SCLC-P |
| chr1: 1396740 | ENST00000400809.8 | CCNL2 | Intron | SCLC-P |
| chr1: 1396771 | ENST00000400809.8 | CCNL2 | Intron | SCLC-P |
| chr1: 1475192 | ENST00000673477.1 | ATAD3B | Intron | SCLC-P |
| chr1: 1475215 | ENST00000673477.1 | ATAD3B | Intron | SCLC-P |
| chr1: 1475335 | ENST00000673477.1 | ATAD3B | Intron | SCLC-P |
| chr1: 1475416 | ENST00000673477.1 | ATAD3B | Intron | SCLC-P |
| chr1: 1475435 | ENST00000673477.1 | ATAD3B | Intron | SCLC-P |
| chr1: 1475454 | ENST00000673477.1 | ATAD3B | Intron | SCLC-P |
| chr1: 1475460 | ENST00000673477.1 | ATAD3B | Intron | SCLC-P |
| chr1: 1475463 | ENST00000673477.1 | ATAD3B | Intron | SCLC-P |
| chr1: 1475470 | ENST00000673477.1 | ATAD3B | Intron | SCLC-P |
| chr1: 1475563 | ENST00000673477.1 | ATAD3B | Intron | SCLC-P |
| chr1: 1534445 | ENST00000378756.8 | ATAD3A | Exon | SCLC-P |
| chr1: 1547615 | ENST00000291386.4 | SSU72 | Intron | SCLC-P |
| chr1: 1549249 | ENST00000291386.4 | SSU72 | Intron | SCLC-P |
| chr1: 1549250 | ENST00000291386.4 | SSU72 | Intron | SCLC-P |
| chr1: 1549259 | ENST00000291386.4 | SSU72 | Intron | SCLC-P |
| chr1: 1549281 | ENST00000291386.4 | SSU72 | Intron | SCLC-P |
| chr1: 1549282 | ENST00000291386.4 | SSU72 | Intron | SCLC-P |
| chr1: 1549287 | ENST00000291386.4 | SSU72 | Intron | SCLC-P |
| chr1: 1549288 | ENST00000291386.4 | SSU72 | Intron | SCLC-P |
| chr1: 1549318 | ENST00000291386.4 | SSU72 | Intron | SCLC-P |
| chr1: 1550663 | ENST00000291386.4 | SSU72 | Intron | SCLC-P |
| chr1: 1554593 | ENST00000291386.4 | SSU72 | Intron | SCLC-P |
| chr1: 1561606 | ENST00000291386.4 | SSU72 | Intron | SCLC-P |
| chr1: 1624522 | ENST00000467597.1 | MIB2 | Promoter | SCLC-P |
| chr1: 1624565 | ENST00000467597.1 | MIB2 | Promoter | SCLC-P |
| chr1: 1624573 | ENST00000467597.1 | MIB2 | Promoter | SCLC-P |
| chr1: 1624618 | ENST00000467597.1 | MIB2 | Promoter | SCLC-P |
| chr1: 1624646 | ENST00000467597.1 | MIB2 | Promoter | SCLC-P |
| chr1: 1683375 | ENST00000611123.1 | SLC35E2B | Intron | SCLC-P |
| chr1: 1765128 | ENST00000341426.9 | NADK | Intron | SCLC-P |
| chr1: 1765131 | ENST00000341426.9 | NADK | Intron | SCLC-P |
| chr1: 1874342 | ENST00000610897.4 | GNB1 | Intron | SCLC-P |
| chr1: 1874344 | ENST00000610897.4 | GNB1 | Intron | SCLC-P |
| chr1: 1923286 | ENST00000642590.1 | CFAP74 | Intron | SCLC-P |
| chr1: 1923287 | ENST00000642590.1 | CFAP74 | Intron | SCLC-P |
| chr1: 1923297 | ENST00000642590.1 | CFAP74 | Intron | SCLC-P |
| chr1: 1923895 | ENST00000642590.1 | CFAP74 | Exon | SCLC-P |
| chr1: 1923913 | ENST00000642590.1 | CFAP74 | Exon | SCLC-P |
| chr1: 2187492 | ENST00000400919.7 | FAAP20 | Intron | SCLC-P |
| chr1: 2187583 | ENST00000400919.7 | FAAP20 | Intron | SCLC-P |
| chr1: 2187584 | ENST00000400919.7 | FAAP20 | Intron | SCLC-P |
| chr1: 2187593 | ENST00000400919.7 | FAAP20 | Intron | SCLC-P |
| chr1: 2187594 | ENST00000400919.7 | FAAP20 | Intron | SCLC-P |
| chr1: 2187595 | ENST00000400919.7 | FAAP20 | Intron | SCLC-P |
| chr1: 2187596 | ENST00000400919.7 | FAAP20 | Intron | SCLC-P |
| chr1: 2187600 | ENST00000400919.7 | FAAP20 | Intron | SCLC-P |
| chr1: 2234472 | ENST00000378536.5 | SKI | Intron | SCLC-P |
| chr1: 2234498 | ENST00000378536.5 | SKI | Intron | SCLC-P |
| chr1: 2317877 | ENST00000661319.1 | NA | Intron | SCLC-P |
| chr1: 2317882 | ENST00000661319.1 | NA | Intron | SCLC-P |
| chr1: 2317884 | ENST00000661319.1 | NA | Intron | SCLC-P |
| chr1: 2324486 | ENST00000378531.8 | MORN1 | Intron | SCLC-P |
| chr1: 2426444 | ENST00000609981.5 | PLCH2 | Intron | SCLC-P |
| chr1: 2427172 | ENST00000609981.5 | PLCH2 | Intron | SCLC-P |
| chr1: 2427361 | ENST00000609981.5 | PLCH2 | Intron | SCLC-P |
| chr1: 2427381 | ENST00000609981.5 | PLCH2 | Intron | SCLC-P |
| chr1: 2502417 | ENST00000473964.5 | PLCH2 | Exon | SCLC-P |
| chr1: 2502425 | ENST00000473964.5 | PLCH2 | Exon | SCLC-P |
| chr1: 2502426 | ENST00000473964.5 | PLCH2 | Exon | SCLC-P |
| chr1: 2502454 | ENST00000473964.5 | PLCH2 | Exon | SCLC-P |
| chr1: 2502455 | ENST00000473964.5 | PLCH2 | Exon | SCLC-P |
| chr1: 2502463 | ENST00000473964.5 | PLCH2 | Exon | SCLC-P |
| chr1: 3087392 | ENST00000511072.5 | PRDM16 | Intron | SCLC-P |
| chr1: 3160979 | ENST00000511072.5 | PRDM16 | Intron | SCLC-P |
| chr1: 3173943 | ENST00000511072.5 | PRDM16 | Intron | SCLC-P |
| chr1: 3173971 | ENST00000511072.5 | PRDM16 | Intron | SCLC-P |
| chr1: 3198355 | ENST00000511072.5 | PRDM16 | Intron | SCLC-P |
| chr1: 3216544 | ENST00000511072.5 | PRDM16 | Intron | SCLC-P |
| chr1: 3216547 | ENST00000511072.5 | PRDM16 | Intron | SCLC-P |
| chr1: 3331596 | ENST00000378391.6 | PRDM16 | Intron | SCLC-P |
| chr1: 3331806 | ENST00000378391.6 | PRDM16 | Intron | SCLC-P |
| chr1: 3357671 | ENST00000378391.6 | PRDM16 | Intron | SCLC-P |
| chr1: 3357683 | ENST00000378391.6 | PRDM16 | Intron | SCLC-P |
| chr1: 3357686 | ENST00000378391.6 | PRDM16 | Intron | SCLC-P |
| chr1: 3357687 | ENST00000378391.6 | PRDM16 | Intron | SCLC-P |
| chr1: 3358718 | ENST00000378391.6 | PRDM16 | Intron | SCLC-P |
| chr1: 3364529 | ENST00000378391.6 | PRDM16 | Intron | SCLC-P |
| chr1: 3364709 | ENST00000378391.6 | PRDM16 | Intron | SCLC-P |
| chr1: 3364710 | ENST00000378391.6 | PRDM16 | Intron | SCLC-P |
| chr1: 3366242 | ENST00000378391.6 | PRDM16 | Intron | SCLC-P |
| chr1: 3366257 | ENST00000378391.6 | PRDM16 | Intron | SCLC-P |
| chr1: 3366414 | ENST00000378391.6 | PRDM16 | Intron | SCLC-P |
| chr1: 3366416 | ENST00000378391.6 | PRDM16 | Intron | SCLC-P |
| chr1: 3367039 | ENST00000378391.6 | PRDM16 | Intron | SCLC-P |
| chr1: 3378512 | ENST00000378391.6 | PRDM16 | Intron | SCLC-P |
| chr1: 3390787 | ENST00000511072.5 | PRDM16 | Intron | SCLC-P |
| chr1: 3391958 | ENST00000511072.5 | PRDM16 | Intron | SCLC-P |
| chr1: 3397625 | ENST00000511072.5 | PRDM16 | Intron | SCLC-P |
| chr1: 3397643 | ENST00000511072.5 | PRDM16 | Intron | SCLC-P |
| chr1: 3405173 | ENST00000511072.5 | PRDM16 | Intron | SCLC-P |
| chr1: 3405197 | ENST00000511072.5 | PRDM16 | Intron | SCLC-P |
| chr1: 3405203 | ENST00000511072.5 | PRDM16 | Intron | SCLC-P |
| chr1: 3405232 | ENST00000511072.5 | PRDM16 | Intron | SCLC-P |
| chr1: 3405254 | ENST00000511072.5 | PRDM16 | Intron | SCLC-P |
| chr1: 3405293 | ENST00000511072.5 | PRDM16 | Intron | SCLC-P |
| chr1: 3405451 | ENST00000511072.5 | PRDM16 | Intron | SCLC-P |
| chr1: 3405485 | ENST00000511072.5 | PRDM16 | Intron | SCLC-P |
| chr1: 3405689 | ENST00000511072.5 | PRDM16 | Intron | SCLC-P |
| chr1: 3405691 | ENST00000511072.5 | PRDM16 | Intron | SCLC-P |
| chr1: 3405702 | ENST00000511072.5 | PRDM16 | Intron | SCLC-P |
| chr1: 3405802 | ENST00000511072.5 | PRDM16 | Intron | SCLC-P |
| chr1: 3405818 | ENST00000511072.5 | PRDM16 | Intron | SCLC-P |
| chr1: 3407090 | ENST00000511072.5 | PRDM16 | Intron | SCLC-P |
| chr1: 3423062 | ENST00000511072.5 | PRDM16 | Intron | SCLC-P |
| chr1: 3425598 | ENST00000606170.1 | PRDM16 | Exon | SCLC-P |
| chr1: 3497141 | ENST00000491842.1 | MEGF6 | Promoter | SCLC-P |
| chr1: 3497522 | ENST00000491842.1 | MEGF6 | Promoter | SCLC-P |
| chr1: 3497894 | ENST00000356575.9 | MEGF6 | Intron | SCLC-P |
| chr1: 3539515 | ENST00000356575.9 | MEGF6 | Intron | SCLC-P |
| chr1: 3539564 | ENST00000356575.9 | MEGF6 | Intron | SCLC-P |
| chr1: 3539694 | ENST00000356575.9 | MEGF6 | Intron | SCLC-P |
| chr1: 3539695 | ENST00000356575.9 | MEGF6 | Intron | SCLC-P |
| chr1: 3539827 | ENST00000356575.9 | MEGF6 | Intron | SCLC-P |
| chr1: 3541769 | ENST00000356575.9 | MEGF6 | Intron | SCLC-P |
| chr1: 3541929 | ENST00000356575.9 | MEGF6 | Intron | SCLC-P |
| chr1: 3541930 | ENST00000356575.9 | MEGF6 | Intron | SCLC-P |
| chr1: 3541945 | ENST00000356575.9 | MEGF6 | Intron | SCLC-P |
| chr1: 3541946 | ENST00000356575.9 | MEGF6 | Intron | SCLC-P |
| chr1: 3552587 | ENST00000356575.9 | MEGF6 | Intron | SCLC-P |
| chr1: 3552608 | ENST00000356575.9 | MEGF6 | Intron | SCLC-P |
| chr1: 3552609 | ENST00000356575.9 | MEGF6 | Intron | SCLC-P |
| chr1: 3552611 | ENST00000356575.9 | MEGF6 | Intron | SCLC-P |
| chr1: 3552612 | ENST00000356575.9 | MEGF6 | Intron | SCLC-P |
| chr1: 3556936 | ENST00000356575.9 | MEGF6 | Intron | SCLC-P |
| chr1: 3567665 | ENST00000356575.9 | MEGF6 | Intron | SCLC-P |
| chr1: 3579918 | ENST00000485002.6 | MEGF6 | Intron | SCLC-P |
| chr1: 3579933 | ENST00000485002.6 | MEGF6 | Intron | SCLC-P |
| chr1: 3581124 | ENST00000485002.6 | MEGF6 | Intron | SCLC-P |
| chr1: 3581129 | ENST00000485002.6 | MEGF6 | Intron | SCLC-P |
| chr1: 3589350 | ENST00000485002.6 | MEGF6 | Intron | SCLC-P |
| chr1: 3589366 | ENST00000485002.6 | MEGF6 | Intron | SCLC-P |
| chr1: 3589367 | ENST00000485002.6 | MEGF6 | Intron | SCLC-P |
| chr1: 3589380 | ENST00000485002.6 | MEGF6 | Intron | SCLC-P |
| chr1: 3589381 | ENST00000485002.6 | MEGF6 | Intron | SCLC-P |
| chr1: 3589384 | ENST00000485002.6 | MEGF6 | Intron | SCLC-P |
| chr1: 3589385 | ENST00000485002.6 | MEGF6 | Intron | SCLC-P |
| chr1: 3591983 | ENST00000485002.6 | MEGF6 | Intron | SCLC-P |
| chr1: 3591990 | ENST00000485002.6 | MEGF6 | Intron | SCLC-P |
| chr1: 3591997 | ENST00000485002.6 | MEGF6 | Intron | SCLC-P |
| chr1: 3592007 | ENST00000485002.6 | MEGF6 | Intron | SCLC-P |
| chr1: 3595668 | ENST00000356575.9 | MEGF6 | Intron | SCLC-P |
| chr1: 3595680 | ENST00000356575.9 | MEGF6 | Intron | SCLC-P |
| chr1: 3595696 | ENST00000356575.9 | MEGF6 | Intron | SCLC-P |
| chr1: 3602483 | ENST00000356575.9 | MEGF6 | Exon | SCLC-P |
| chr1: 3602519 | ENST00000356575.9 | MEGF6 | Exon | SCLC-P |
| chr1: 3602525 | ENST00000356575.9 | MEGF6 | Exon | SCLC-P |
| chr1: 3602545 | ENST00000356575.9 | MEGF6 | Exon | SCLC-P |
| chr1: 3602549 | ENST00000356575.9 | MEGF6 | Exon | SCLC-P |
| chr1: 3602556 | ENST00000356575.9 | MEGF6 | Exon | SCLC-P |
| chr1: 3602559 | ENST00000356575.9 | MEGF6 | Exon | SCLC-P |
| chr1: 3605977 | ENST00000356575.9 | MEGF6 | Intron | SCLC-P |
| chr1: 3606636 | ENST00000356575.9 | MEGF6 | Intron | SCLC-P |
| chr1: 3673669 | ENST00000378295.9 | TP73 | Intron | SCLC-P |
| chr1: 3673674 | ENST00000378295.9 | TP73 | Intron | SCLC-P |
| chr1: 3673802 | ENST00000378295.9 | TP73 | Intron | SCLC-P |
| chr1: 3679014 | ENST00000378295.9 | TP73 | Intron | SCLC-P |
| chr1: 3679030 | ENST00000378295.9 | TP73 | Intron | SCLC-P |
| chr1: 3679034 | ENST00000378295.9 | TP73 | Intron | SCLC-P |
| chr1: 3682353 | ENST00000603362.5 | TP73 | Promoter | SCLC-P |
| chr1: 3682356 | ENST00000603362.5 | TP73 | Promoter | SCLC-P |
| chr1: 3682380 | ENST00000378295.9 | TP73 | Exon | SCLC-P |
| chr1: 3683938 | ENST00000378295.9 | TP73 | Intron | SCLC-P |
| chr1: 3684046 | ENST00000378295.9 | TP73 | Intron | SCLC-P |
| chr1: 3684085 | ENST00000378295.9 | TP73 | Intron | SCLC-P |
| chr1: 3701698 | ENST00000378295.9 | TP73 | Intron | SCLC-P |
| chr1: 3704685 | ENST00000378295.9 | TP73 | Intron | SCLC-P |
| chr1: 3704725 | ENST00000378295.9 | TP73 | Intron | SCLC-P |
| chr1: 3704744 | ENST00000378295.9 | TP73 | Intron | SCLC-P |
| chr1: 3704745 | ENST00000378295.9 | TP73 | Intron | SCLC-P |
| chr1: 3704882 | ENST00000378295.9 | TP73 | Intron | SCLC-P |
| chr1: 3705781 | ENST00000378295.9 | TP73 | Intron | SCLC-P |
| chr1: 3705782 | ENST00000378295.9 | TP73 | Intron | SCLC-P |
| chr1: 3705842 | ENST00000378295.9 | TP73 | Intron | SCLC-P |
| chr1: 3705849 | ENST00000378295.9 | TP73 | Intron | SCLC-P |
| chr1: 3705997 | ENST00000378295.9 | TP73 | Intron | SCLC-P |
| chr1: 3706755 | ENST00000378295.9 | TP73 | Intron | SCLC-P |
| chr1: 3706769 | ENST00000378295.9 | TP73 | Intron | SCLC-P |
| chr1: 3706775 | ENST00000378295.9 | TP73 | Intron | SCLC-P |
| chr1: 3706905 | ENST00000378295.9 | TP73 | Intron | SCLC-P |
| chr1: 3706906 | ENST00000378295.9 | TP73 | Intron | SCLC-P |
| chr1: 3706912 | ENST00000378295.9 | TP73 | Intron | SCLC-P |
| chr1: 3706913 | ENST00000378295.9 | TP73 | Intron | SCLC-P |
| chr1: 3706926 | ENST00000378295.9 | TP73 | Intron | SCLC-P |
| chr1: 3706937 | ENST00000378295.9 | TP73 | Intron | SCLC-P |
| chr1: 3707095 | ENST00000378295.9 | TP73 | Intron | SCLC-P |
| chr1: 3707423 | ENST00000378295.9 | TP73 | Intron | SCLC-P |
| chr1: 3722142 | ENST00000378295.9 | TP73 | Exon | SCLC-P |
| chr1: 3727277 | ENST00000604566.1 | TP73 | Promoter | SCLC-P |
| chr1: 3733380 | ENST00000378295.9 | TP73 | Exon | SCLC-P |
| chr1: 3733407 | ENST00000378295.9 | TP73 | Exon | SCLC-P |
| chr1: 3733595 | ENST00000378295.9 | TP73 | Exon | SCLC-P |
| chr1: 3733604 | ENST00000378295.9 | TP73 | Exon | SCLC-P |
| chr1: 3734365 | ENST00000378295.9 | TP73 | Exon | SCLC-P |
| chr1: 3752708 | ENST00000462521.2 | CCDC27 | Exon | SCLC-P |
| chr1: 3752716 | ENST00000462521.2 | CCDC27 | Exon | SCLC-P |
| chr1: 3752735 | ENST00000462521.2 | CCDC27 | Exon | SCLC-P |
| chr1: 3752783 | ENST00000462521.2 | CCDC27 | Exon | SCLC-P |
| chr1: 3763125 | ENST00000462521.2 | CCDC27 | Exon | SCLC-P |
| chr1: 3763130 | ENST00000462521.2 | CCDC27 | Exon | SCLC-P |
| chr1: 3763131 | ENST00000462521.2 | CCDC27 | Exon | SCLC-P |
| chr1: 3912101 | ENST00000667408.1 | LINC01134 | Intron | SCLC-P |
| chr1: 5651349 | ENST00000637702.1 | NA | Intron | SCLC-P |
| chr1: 6019912 | ENST00000655548.1 | KCNAB2 | Intron | SCLC-P |
| chr1: 6019918 | ENST00000655548.1 | KCNAB2 | Intron | SCLC-P |
| chr1: 6047242 | ENST00000389632.9 | KCNAB2 | Intron | SCLC-P |
| chr1: 6048447 | ENST00000389632.9 | KCNAB2 | Intron | SCLC-P |
| chr1: 6056292 | ENST00000389632.9 | KCNAB2 | Intron | SCLC-P |
| chr1: 6056296 | ENST00000389632.9 | KCNAB2 | Intron | SCLC-P |
| chr1: 6056297 | ENST00000389632.9 | KCNAB2 | Intron | SCLC-P |
| chr1: 6056324 | ENST00000389632.9 | KCNAB2 | Intron | SCLC-P |
| chr1: 6056335 | ENST00000389632.9 | KCNAB2 | Intron | SCLC-P |
| chr1: 6056336 | ENST00000389632.9 | KCNAB2 | Intron | SCLC-P |
| chr1: 6056337 | ENST00000389632.9 | KCNAB2 | Intron | SCLC-P |
| chr1: 6056340 | ENST00000389632.9 | KCNAB2 | Intron | SCLC-P |
| chr1: 6056341 | ENST00000389632.9 | KCNAB2 | Intron | SCLC-P |
| chr1: 6059746 | ENST00000389632.9 | KCNAB2 | Intron | SCLC-P |
| chr1: 6087287 | ENST00000655548.1 | KCNAB2 | Intron | SCLC-P |
| chr1: 6087288 | ENST00000655548.1 | KCNAB2 | Intron | SCLC-P |
| chr1: 6087294 | ENST00000655548.1 | KCNAB2 | Intron | SCLC-P |
| chr1: 6087295 | ENST00000655548.1 | KCNAB2 | Intron | SCLC-P |
| chr1: 6089479 | ENST00000655548.1 | KCNAB2 | Intron | SCLC-P |
| chr1: 6090587 | ENST00000378097.6 | KCNAB2 | Intron | SCLC-P |
| chr1: 6090600 | ENST00000378097.6 | KCNAB2 | Intron | SCLC-P |
| chr1: 6095342 | ENST00000652911.1 | KCNAB2 | Exon | SCLC-P |
| chr1: 6100315 | ENST00000662815.1 | KCNAB2 | Exon | SCLC-P |
| chr1: 6106506 | ENST00000475121.1 | CHD5 | Intron | SCLC-P |
| chr1: 6106674 | ENST00000262450.8 | CHD5 | Exon | SCLC-P |
| chr1: 6125548 | ENST00000496404.1 | CHD5 | Intron | SCLC-P |
| chr1: 6125555 | ENST00000496404.1 | CHD5 | Intron | SCLC-P |
| chr1: 6125595 | ENST00000496404.1 | CHD5 | Intron | SCLC-P |
| chr1: 6125777 | ENST00000496404.1 | CHD5 | Intron | SCLC-P |
| chr1: 6128791 | ENST00000496404.1 | CHD5 | Intron | SCLC-P |
| chr1: 6128814 | ENST00000496404.1 | CHD5 | Intron | SCLC-P |
| chr1: 6135308 | ENST00000262450.8 | CHD5 | Exon | SCLC-P |
| chr1: 6135322 | ENST00000262450.8 | CHD5 | Exon | SCLC-P |
| chr1: 6136178 | ENST00000262450.8 | CHD5 | Intron | SCLC-P |
| chr1: 6145994 | ENST00000262450.8 | CHD5 | Intron | SCLC-P |
| chr1: 6146008 | ENST00000262450.8 | CHD5 | Intron | SCLC-P |
| chr1: 6146131 | ENST00000262450.8 | CHD5 | Intron | SCLC-P |
| chr1: 6146789 | ENST00000262450.8 | CHD5 | Exon | SCLC-P |
| chr1: 6161927 | ENST00000262450.8 | CHD5 | Intron | SCLC-P |
| chr1: 6161939 | ENST00000262450.8 | CHD5 | Intron | SCLC-P |
| chr1: 6161959 | ENST00000262450.8 | CHD5 | Intron | SCLC-P |
| chr1: 6165907 | ENST00000262450.8 | CHD5 | Intron | SCLC-P |
| chr1: 6210726 | ENST00000463453.1 | RNF207 | Promoter | SCLC-P |
| chr1: 6211967 | ENST00000483336.1 | RNF207 | Promoter | SCLC-P |
| chr1: 6222880 | ENST00000343813.10 | ICMT | Exon | SCLC-P |
| chr1: 6236574 | ENST00000650463.1 | LINC00337 | Intron | SCLC-P |
| chr1: 6236582 | ENST00000650463.1 | LINC00337 | Intron | SCLC-P |
| chr1: 6236585 | ENST00000650463.1 | LINC00337 | Intron | SCLC-P |
| chr1: 6236593 | ENST00000650463.1 | LINC00337 | Intron | SCLC-P |
| chr1: 6236596 | ENST00000650463.1 | LINC00337 | Intron | SCLC-P |
| chr1: 6236597 | ENST00000650463.1 | LINC00337 | Intron | SCLC-P |
| chr1: 6236676 | ENST00000650463.1 | LINC00337 | Intron | SCLC-P |
| chr1: 6236710 | ENST00000650463.1 | LINC00337 | Intron | SCLC-P |
| chr1: 6236851 | ENST00000650463.1 | LINC00337 | Intron | SCLC-P |
| chr1: 6236867 | ENST00000650463.1 | LINC00337 | Intron | SCLC-P |
| chr1: 6236927 | ENST00000650463.1 | LINC00337 | Intron | SCLC-P |
| chr1: 6236938 | ENST00000650463.1 | LINC00337 | Intron | SCLC-P |
| chr1: 6236973 | ENST00000650463.1 | LINC00337 | Intron | SCLC-P |
| chr1: 6236983 | ENST00000650463.1 | LINC00337 | Intron | SCLC-P |
| chr1: 6236993 | ENST00000650463.1 | LINC00337 | Intron | SCLC-P |
| chr1: 6430838 | ENST00000645284.1 | ESPN | Intron | SCLC-P |
| chr1: 6451836 | ENST00000477679.2 | ESPN | Exon | SCLC-P |
| chr1: 6460544 | ENST00000645284.1 | ESPN | Exon | SCLC-P |
| chr1: 6460550 | ENST00000645284.1 | ESPN | Exon | SCLC-P |
| chr1: 6460619 | ENST00000645284.1 | ESPN | Exon | SCLC-P |
| chr1: 6460623 | ENST00000645284.1 | ESPN | Exon | SCLC-P |
| chr1: 6460654 | ENST00000645284.1 | ESPN | Exon | SCLC-P |
| chr1: 6460770 | ENST00000645284.1 | ESPN | Exon | SCLC-P |
| chr1: 6460775 | ENST00000645284.1 | ESPN | Exon | SCLC-P |
| chr1: 6460795 | ENST00000645284.1 | ESPN | Exon | SCLC-P |
| chr1: 6460801 | ENST00000645284.1 | ESPN | Exon | SCLC-P |
| chr1: 6460819 | ENST00000645284.1 | ESPN | Exon | SCLC-P |
| chr1: 6460881 | ENST00000645284.1 | ESPN | Exon | SCLC-P |
| chr1: 6460882 | ENST00000645284.1 | ESPN | Exon | SCLC-P |
| chr1: 6460893 | ENST00000645284.1 | ESPN | Exon | SCLC-P |
| chr1: 6460901 | ENST00000645284.1 | ESPN | Exon | SCLC-P |
| chr1: 6460917 | ENST00000645284.1 | ESPN | Exon | SCLC-P |
| chr1: 6460918 | ENST00000645284.1 | ESPN | Exon | SCLC-P |
| chr1: 6460934 | ENST00000645284.1 | ESPN | Exon | SCLC-P |
| chr1: 6460935 | ENST00000645284.1 | ESPN | Exon | SCLC-P |
| chr1: 6460948 | ENST00000434576.2 | ESPN | Intron | SCLC-P |
| chr1: 6460957 | ENST00000434576.2 | ESPN | Intron | SCLC-P |
| chr1: 6460958 | ENST00000434576.2 | ESPN | Intron | SCLC-P |
| chr1: 6460959 | ENST00000434576.2 | ESPN | Intron | SCLC-P |
| chr1: 6461061 | ENST00000434576.2 | ESPN | Intron | SCLC-P |
| chr1: 6461062 | ENST00000434576.2 | ESPN | Intron | SCLC-P |
| chr1: 6461078 | ENST00000434576.2 | ESPN | Intron | SCLC-P |
| chr1: 6461087 | ENST00000434576.2 | ESPN | Intron | SCLC-P |
| chr1: 6461096 | ENST00000434576.2 | ESPN | Intron | SCLC-P |
| chr1: 6461192 | ENST00000647810.1 | TNFRSF25 | Exon | SCLC-P |
| chr1: 6461209 | ENST00000647810.1 | TNFRSF25 | Exon | SCLC-P |
| chr1: 6461243 | ENST00000647810.1 | TNFRSF25 | Exon | SCLC-P |
| chr1: 6461250 | ENST00000647810.1 | TNFRSF25 | Exon | SCLC-P |
| chr1: 6461254 | ENST00000647810.1 | TNFRSF25 | Exon | SCLC-P |
| chr1: 6461265 | ENST00000647810.1 | TNFRSF25 | Exon | SCLC-P |
| chr1: 6461279 | ENST00000647810.1 | TNFRSF25 | Exon | SCLC-P |
| chr1: 6461288 | ENST00000647810.1 | TNFRSF25 | Exon | SCLC-P |
| chr1: 6461304 | ENST00000647810.1 | TNFRSF25 | Exon | SCLC-P |
| chr1: 6471003 | ENST00000487949.4 | PLEKHG5 | Exon | SCLC-P |
| chr1: 6471019 | ENST00000487949.4 | PLEKHG5 | Exon | SCLC-P |
| chr1: 6471021 | ENST00000487949.4 | PLEKHG5 | Exon | SCLC-P |
| chr1: 6471022 | ENST00000487949.4 | PLEKHG5 | Exon | SCLC-P |
| chr1: 6471033 | ENST00000487949.4 | PLEKHG5 | Exon | SCLC-P |
| chr1: 6471034 | ENST00000487949.4 | PLEKHG5 | Exon | SCLC-P |
| chr1: 6471072 | ENST00000487949.4 | PLEKHG5 | Exon | SCLC-P |
| chr1: 6471092 | ENST00000487949.4 | PLEKHG5 | Exon | SCLC-P |
| chr1: 6471097 | ENST00000487949.4 | PLEKHG5 | Exon | SCLC-P |
| chr1: 6471160 | ENST00000487949.4 | PLEKHG5 | Exon | SCLC-P |
| chr1: 6471162 | ENST00000487949.4 | PLEKHG5 | Exon | SCLC-P |
| chr1: 6471169 | ENST00000487949.4 | PLEKHG5 | Exon | SCLC-P |
| chr1: 6471174 | ENST00000487949.4 | PLEKHG5 | Exon | SCLC-P |
| chr1: 6471179 | ENST00000487949.4 | PLEKHG5 | Exon | SCLC-P |
| chr1: 6471180 | ENST00000487949.4 | PLEKHG5 | Exon | SCLC-P |
| chr1: 6471200 | ENST00000487949.4 | PLEKHG5 | Exon | SCLC-P |
| chr1: 6905286 | ENST00000303635.12 | CAMTA1 | Intron | SCLC-P |
| chr1: 6905292 | ENST00000303635.12 | CAMTA1 | Intron | SCLC-P |
| chr1: 6916294 | ENST00000303635.12 | CAMTA1 | Intron | SCLC-P |
| chr1: 6961465 | ENST00000303635.12 | CAMTA1 | Intron | SCLC-P |
| chr1: 6961475 | ENST00000303635.12 | CAMTA1 | Intron | SCLC-P |
| chr1: 6961486 | ENST00000303635.12 | CAMTA1 | Intron | SCLC-P |
| chr1: 6961552 | ENST00000303635.12 | CAMTA1 | Intron | SCLC-P |
| chr1: 7049116 | ENST00000303635.12 | CAMTA1 | Intron | SCLC-P |
| chr1: 7049123 | ENST00000303635.12 | CAMTA1 | Intron | SCLC-P |
| chr1: 7062579 | ENST00000303635.12 | CAMTA1 | Intron | SCLC-P |
| chr1: 7062580 | ENST00000303635.12 | CAMTA1 | Intron | SCLC-P |
| chr1: 7062581 | ENST00000303635.12 | CAMTA1 | Intron | SCLC-P |
| chr1: 7068684 | ENST00000303635.12 | CAMTA1 | Intron | SCLC-P |
| chr1: 7090119 | ENST00000303635.12 | CAMTA1 | Intron | SCLC-P |
| chr1: 7096454 | ENST00000303635.12 | CAMTA1 | Intron | SCLC-P |
| chr1: 7131777 | ENST00000303635.12 | CAMTA1 | Intron | SCLC-P |
| chr1: 7131793 | ENST00000303635.12 | CAMTA1 | Intron | SCLC-P |
| chr1: 7131821 | ENST00000303635.12 | CAMTA1 | Intron | SCLC-P |
| chr1: 7131826 | ENST00000303635.12 | CAMTA1 | Intron | SCLC-P |
| chr1: 7145731 | ENST00000303635.12 | CAMTA1 | Intron | SCLC-P |
| chr1: 7146227 | ENST00000303635.12 | CAMTA1 | Intron | SCLC-P |
| chr1: 7146256 | ENST00000303635.12 | CAMTA1 | Intron | SCLC-P |
| chr1: 7155183 | ENST00000303635.12 | CAMTA1 | Intron | SCLC-P |
| chr1: 7214613 | ENST00000303635.12 | CAMTA1 | Intron | SCLC-P |
| chr1: 7236555 | ENST00000303635.12 | CAMTA1 | Intron | SCLC-P |
| chr1: 7236566 | ENST00000303635.12 | CAMTA1 | Intron | SCLC-P |
| chr1: 7237778 | ENST00000303635.12 | CAMTA1 | Intron | SCLC-P |
| chr1: 7259296 | ENST00000303635.12 | CAMTA1 | Intron | SCLC-P |
| chr1: 7347129 | ENST00000303635.12 | CAMTA1 | Intron | SCLC-P |
| chr1: 7347138 | ENST00000303635.12 | CAMTA1 | Intron | SCLC-P |
| chr1: 7347156 | ENST00000303635.12 | CAMTA1 | Intron | SCLC-P |
| chr1: 7347168 | ENST00000303635.12 | CAMTA1 | Intron | SCLC-P |
| chr1: 7347171 | ENST00000303635.12 | CAMTA1 | Intron | SCLC-P |
| chr1: 7347251 | ENST00000303635.12 | CAMTA1 | Intron | SCLC-P |
| chr1: 7351249 | ENST00000303635.12 | CAMTA1 | Intron | SCLC-P |
| chr1: 7408068 | ENST00000303635.12 | CAMTA1 | Intron | SCLC-P |
| chr1: 7408095 | ENST00000303635.12 | CAMTA1 | Intron | SCLC-P |
| chr1: 7408210 | ENST00000303635.12 | CAMTA1 | Intron | SCLC-P |
| chr1: 7432317 | ENST00000303635.12 | CAMTA1 | Intron | SCLC-P |
| chr1: 7432451 | ENST00000303635.12 | CAMTA1 | Intron | SCLC-P |
| chr1: 7432617 | ENST00000303635.12 | CAMTA1 | Intron | SCLC-P |
| chr1: 7432621 | ENST00000303635.12 | CAMTA1 | Intron | SCLC-P |
| chr1: 7502058 | ENST00000303635.12 | CAMTA1 | Intron | SCLC-P |
| chr1: 7502152 | ENST00000303635.12 | CAMTA1 | Intron | SCLC-P |
| chr1: 7503063 | ENST00000303635.12 | CAMTA1 | Intron | SCLC-P |
| chr1: 7513634 | ENST00000303635.12 | CAMTA1 | Intron | SCLC-P |
| chr1: 7524859 | ENST00000303635.12 | CAMTA1 | Intron | SCLC-P |
| chr1: 7524905 | ENST00000303635.12 | CAMTA1 | Intron | SCLC-P |
| chr1: 7531615 | ENST00000303635.12 | CAMTA1 | Intron | SCLC-P |
| chr1: 7533109 | ENST00000303635.12 | CAMTA1 | Intron | SCLC-P |
| chr1: 7535115 | ENST00000303635.12 | CAMTA1 | Intron | SCLC-P |
| chr1: 7550312 | ENST00000303635.12 | CAMTA1 | Intron | SCLC-P |
| chr1: 7550446 | ENST00000303635.12 | CAMTA1 | Intron | SCLC-P |
| chr1: 7557372 | ENST00000303635.12 | CAMTA1 | Intron | SCLC-P |
| chr1: 7557380 | ENST00000303635.12 | CAMTA1 | Intron | SCLC-P |
| chr1: 7557491 | ENST00000303635.12 | CAMTA1 | Intron | SCLC-P |
| chr1: 7573817 | ENST00000303635.12 | CAMTA1 | Intron | SCLC-P |
| chr1: 7578510 | ENST00000303635.12 | CAMTA1 | Intron | SCLC-P |
| chr1: 7578519 | ENST00000303635.12 | CAMTA1 | Intron | SCLC-P |
| chr1: 7578528 | ENST00000303635.12 | CAMTA1 | Intron | SCLC-P |
| chr1: 7578531 | ENST00000303635.12 | CAMTA1 | Intron | SCLC-P |
| chr1: 7583492 | ENST00000303635.12 | CAMTA1 | Intron | SCLC-P |
| chr1: 7585015 | ENST00000303635.12 | CAMTA1 | Intron | SCLC-P |
| chr1: 7611708 | ENST00000303635.12 | CAMTA1 | Intron | SCLC-P |
| chr1: 7613020 | ENST00000303635.12 | CAMTA1 | Intron | SCLC-P |
| chr1: 7616494 | ENST00000303635.12 | CAMTA1 | Intron | SCLC-P |
| chr1: 7629361 | ENST00000303635.12 | CAMTA1 | Intron | SCLC-P |
| chr1: 7633574 | ENST00000303635.12 | CAMTA1 | Intron | SCLC-P |
| chr1: 7635642 | ENST00000303635.12 | CAMTA1 | Intron | SCLC-P |
| chr1: 7635659 | ENST00000303635.12 | CAMTA1 | Intron | SCLC-P |
| chr1: 7640406 | ENST00000303635.12 | CAMTA1 | Exon | SCLC-P |
| chr1: 7640412 | ENST00000303635.12 | CAMTA1 | Exon | SCLC-P |
| chr1: 7640433 | ENST00000303635.12 | CAMTA1 | Exon | SCLC-P |
| chr1: 7640649 | ENST00000303635.12 | CAMTA1 | Intron | SCLC-P |
| chr1: 7640650 | ENST00000303635.12 | CAMTA1 | Intron | SCLC-P |
| chr1: 7640655 | ENST00000303635.12 | CAMTA1 | Intron | SCLC-P |
| chr1: 7640656 | ENST00000303635.12 | CAMTA1 | Intron | SCLC-P |
| chr1: 7640688 | ENST00000303635.12 | CAMTA1 | Intron | SCLC-P |
| chr1: 7640689 | ENST00000303635.12 | CAMTA1 | Intron | SCLC-P |
| chr1: 7642459 | ENST00000303635.12 | CAMTA1 | Intron | SCLC-P |
| chr1: 7642467 | ENST00000303635.12 | CAMTA1 | Intron | SCLC-P |
| chr1: 7642468 | ENST00000303635.12 | CAMTA1 | Intron | SCLC-P |
| chr1: 7642486 | ENST00000303635.12 | CAMTA1 | Intron | SCLC-P |
| chr1: 7642487 | ENST00000303635.12 | CAMTA1 | Intron | SCLC-P |
| chr1: 7642500 | ENST00000303635.12 | CAMTA1 | Intron | SCLC-P |
| chr1: 7648492 | ENST00000303635.12 | CAMTA1 | Intron | SCLC-P |
| chr1: 7648558 | ENST00000303635.12 | CAMTA1 | Intron | SCLC-P |
| chr1: 7648631 | ENST00000303635.12 | CAMTA1 | Intron | SCLC-P |
| chr1: 7660832 | ENST00000303635.12 | CAMTA1 | Intron | SCLC-P |
| chr1: 7686766 | ENST00000303635.12 | CAMTA1 | Intron | SCLC-P |
| chr1: 7688304 | ENST00000303635.12 | CAMTA1 | Intron | SCLC-P |
| chr1: 7991044 | ENST00000641354.1 | NA | Promoter | SCLC-P |
| chr1: 8201287 | ENST00000670361.1 | LINC01714 | Promoter | SCLC-P |
| chr1: 8211168 | ENST00000670361.1 | LINC01714 | Intron | SCLC-P |
| chr1: 8211188 | ENST00000670361.1 | LINC01714 | Intron | SCLC-P |
| chr1: 8324735 | ENST00000471889.5 | SLC45A1 | Intron | SCLC-P |
| chr1: 8324746 | ENST00000471889.5 | SLC45A1 | Intron | SCLC-P |
| chr1: 8339445 | ENST00000471889.5 | SLC45A1 | Intron | SCLC-P |
| chr1: 8339458 | ENST00000471889.5 | SLC45A1 | Intron | SCLC-P |
| chr1: 8339529 | ENST00000471889.5 | SLC45A1 | Exon | SCLC-P |
| chr1: 8339555 | ENST00000471889.5 | SLC45A1 | Exon | SCLC-P |
| chr1: 8339561 | ENST00000471889.5 | SLC45A1 | Exon | SCLC-P |
| chr1: 8339573 | ENST00000471889.5 | SLC45A1 | Exon | SCLC-P |
| chr1: 8395192 | ENST00000337907.7 | RERE | Intron | SCLC-P |
| chr1: 8515383 | ENST00000507012.1 | RERE | Intron | SCLC-P |
| chr1: 8610066 | ENST00000514428.1 | RERE | Intron | SCLC-P |
| chr1: 8871259 | ENST00000646680.1 | ENO1 | Intron | SCLC-P |
| chr1: 8879289 | ENST00000643438.1 | ENO1 | Promoter | SCLC-P |
| chr1: 9320710 | ENST00000328089.11 | SPSB1 | Intron | SCLC-P |
| chr1: 9320840 | ENST00000328089.11 | SPSB1 | Intron | SCLC-P |
| chr1: 9320847 | ENST00000328089.11 | SPSB1 | Intron | SCLC-P |
| chr1: 9320850 | ENST00000328089.11 | SPSB1 | Intron | SCLC-P |
| chr1: 9541107 | ENST00000302692.7 | SLC25A33 | Intron | SCLC-P |
| chr1: 9541129 | ENST00000302692.7 | SLC25A33 | Intron | SCLC-P |
| chr1: 9541130 | ENST00000302692.7 | SLC25A33 | Intron | SCLC-P |
| chr1: 9541177 | ENST00000302692.7 | SLC25A33 | Intron | SCLC-P |
| chr1: 9541184 | ENST00000302692.7 | SLC25A33 | Intron | SCLC-P |
| chr1: 9541185 | ENST00000302692.7 | SLC25A33 | Intron | SCLC-P |
| chr1: 9541214 | ENST00000302692.7 | SLC25A33 | Intron | SCLC-P |
| chr1: 9541215 | ENST00000302692.7 | SLC25A33 | Intron | SCLC-P |
| chr1: 9541239 | ENST00000302692.7 | SLC25A33 | Intron | SCLC-P |
| chr1: 9541240 | ENST00000302692.7 | SLC25A33 | Intron | SCLC-P |
| chr1: 9541246 | ENST00000302692.7 | SLC25A33 | Intron | SCLC-P |
| chr1: 9541247 | ENST00000302692.7 | SLC25A33 | Intron | SCLC-P |
| chr1: 9541254 | ENST00000302692.7 | SLC25A33 | Intron | SCLC-P |
| chr1: 9541278 | ENST00000302692.7 | SLC25A33 | Intron | SCLC-P |
| chr1: 9541302 | ENST00000302692.7 | SLC25A33 | Intron | SCLC-P |
| chr1: 9541444 | ENST00000302692.7 | SLC25A33 | Intron | SCLC-P |
| chr1: 9541450 | ENST00000302692.7 | SLC25A33 | Intron | SCLC-P |
| chr1: 9541485 | ENST00000302692.7 | SLC25A33 | Intron | SCLC-P |
| chr1: 9542000 | ENST00000302692.7 | SLC25A33 | Intron | SCLC-P |
| chr1: 9949211 | ENST00000445884.1 | NMNAT1 | Intron | SCLC-P |
| chr1: 10399714 | ENST00000487775.1 | PGD | Exon | SCLC-P |
| chr1: 10491872 | ENST00000472851.1 | PEX14 | Intron | SCLC-P |
| chr1: 10614772 | ENST00000356607.9 | PEX14 | Intron | SCLC-P |
| chr1: 10614960 | ENST00000356607.9 | PEX14 | Intron | SCLC-P |
| chr1: 10638287 | ENST00000377022.8 | CASZ1 | Exon | SCLC-P |
| chr1: 10638331 | ENST00000377022.8 | CASZ1 | Exon | SCLC-P |
| chr1: 10643732 | ENST00000606802.1 | NA | Intron | SCLC-P |
| chr1: 10666482 | ENST00000377022.8 | CASZ1 | Intron | SCLC-P |
| chr1: 10666544 | ENST00000377022.8 | CASZ1 | Intron | SCLC-P |
| chr1: 11027945 | ENST00000614757.4 | TARDBP | Intron | SCLC-P |
| chr1: 11032484 | ENST00000400897.8 | MASP2 | Intron | SCLC-P |
| chr1: 11033047 | ENST00000400897.8 | MASP2 | Intron | SCLC-P |
| chr1: 11033051 | ENST00000400897.8 | MASP2 | Intron | SCLC-P |
| chr1: 11519397 | ENST00000294484.7 | DISP3 | Exon | SCLC-P |
| chr1: 11519418 | ENST00000294484.7 | DISP3 | Exon | SCLC-P |
| chr1: 11520613 | ENST00000294484.7 | DISP3 | Intron | SCLC-P |
| chr1: 11521111 | ENST00000294484.7 | DISP3 | Intron | SCLC-P |
| chr1: 11529915 | ENST00000294484.7 | DISP3 | Exon | SCLC-P |
| chr1: 11529945 | ENST00000294484.7 | DISP3 | Exon | SCLC-P |
| chr1: 11531203 | ENST00000294484.7 | DISP3 | Intron | SCLC-P |
| chr1: 11531653 | ENST00000294484.7 | DISP3 | Exon | SCLC-P |
| chr1: 11531665 | ENST00000294484.7 | DISP3 | Exon | SCLC-P |
| chr1: 11531678 | ENST00000294484.7 | DISP3 | Exon | SCLC-P |
| chr1: 11531680 | ENST00000294484.7 | DISP3 | Exon | SCLC-P |
| chr1: 11531710 | ENST00000294484.7 | DISP3 | Exon | SCLC-P |
| chr1: 11531746 | ENST00000294484.7 | DISP3 | Intron | SCLC-P |
| chr1: 11531756 | ENST00000294484.7 | DISP3 | Intron | SCLC-P |
| chr1: 11532110 | ENST00000294484.7 | DISP3 | Intron | SCLC-P |
| chr1: 11534489 | ENST00000294484.7 | DISP3 | Exon | SCLC-P |
| chr1: 11534506 | ENST00000294484.7 | DISP3 | Exon | SCLC-P |
| chr1: 11534536 | ENST00000294484.7 | DISP3 | Exon | SCLC-P |
| chr1: 11534600 | ENST00000294484.7 | DISP3 | Intron | SCLC-P |
| chr1: 11534660 | ENST00000294484.7 | DISP3 | Intron | SCLC-P |
| chr1: 11534828 | ENST00000294484.7 | DISP3 | Intron | SCLC-P |
| chr1: 11536648 | ENST00000294484.7 | DISP3 | Exon | SCLC-P |
| chr1: 11536669 | ENST00000294484.7 | DISP3 | Exon | SCLC-P |
| chr1: 11536690 | ENST00000294484.7 | DISP3 | Exon | SCLC-P |
| chr1: 11536894 | ENST00000294484.7 | DISP3 | Exon | SCLC-P |
| chr1: 11635902 | ENST00000642131.1 | NA | Intron | SCLC-P |
| chr1: 11636023 | ENST00000642131.1 | NA | Intron | SCLC-P |
| chr1: 11636036 | ENST00000642131.1 | NA | Intron | SCLC-P |
| chr1: 11638777 | ENST00000642131.1 | NA | Intron | SCLC-P |
| chr1: 11648235 | ENST00000642025.1 | FBXO2 | Exon | SCLC-P |
| chr1: 11648320 | ENST00000642025.1 | FBXO2 | Exon | SCLC-P |
| chr1: 11650911 | ENST00000471501.1 | FBXO2 | Promoter | SCLC-P |
| chr1: 11692562 | ENST00000376655.1 | MAD2L2 | Promoter | SCLC-P |
| chr1: 11765872 | ENST00000484153.1 | C1orf167 | Promoter | SCLC-P |
| chr1: 11766722 | ENST00000484153.1 | C1orf167 | Promoter | SCLC-P |
| chr1: 11858331 | ENST00000376468.4 | NPPB | Exon | SCLC-P |
| chr1: 11858340 | ENST00000376468.4 | NPPB | Exon | SCLC-P |
| chr1: 11858350 | ENST00000376468.4 | NPPB | Exon | SCLC-P |
| chr1: 11858364 | ENST00000376468.4 | NPPB | Exon | SCLC-P |
| chr1: 11878068 | ENST00000438808.1 | NA | Exon | SCLC-P |
| chr1: 11878785 | ENST00000438808.1 | NA | Exon | SCLC-P |
| chr1: 11878943 | ENST00000438808.1 | NA | Exon | SCLC-P |
| chr1: 11937143 | ENST00000485046.5 | PLOD1 | Intron | SCLC-P |
| chr1: 11937178 | ENST00000485046.5 | PLOD1 | Intron | SCLC-P |
| chr1: 11937204 | ENST00000485046.5 | PLOD1 | Intron | SCLC-P |
| chr1: 11937270 | ENST00000485046.5 | PLOD1 | Intron | SCLC-P |
| chr1: 11937286 | ENST00000485046.5 | PLOD1 | Intron | SCLC-P |
| chr1: 11937599 | ENST00000485046.5 | PLOD1 | Intron | SCLC-P |
| chr1: 11937621 | ENST00000485046.5 | PLOD1 | Intron | SCLC-P |
| chr1: 11941809 | ENST00000485046.5 | PLOD1 | Intron | SCLC-P |
| chr1: 12020671 | ENST00000478749.5 | MIIP | Intron | SCLC-P |
| chr1: 12020706 | ENST00000478749.5 | MIIP | Intron | SCLC-P |
| chr1: 12141637 | ENST00000479933.2 | TNFRSF8 | Intron | SCLC-P |
| chr1: 12143533 | ENST00000263932.7 | TNFRSF8 | Exon | SCLC-P |
| chr1: 12143563 | ENST00000263932.7 | TNFRSF8 | Exon | SCLC-P |
| chr1: 12143575 | ENST00000263932.7 | TNFRSF8 | Exon | SCLC-P |
| chr1: 13490926 | ENST00000376085.4 | LRRC38 | Intron | SCLC-P |
| chr1: 14971957 | ENST00000636203.1 | KAZN | Intron | SCLC-P |
| chr1: 15091510 | ENST00000636203.1 | KAZN | Intron | SCLC-P |
| chr1: 15091551 | ENST00000636203.1 | KAZN | Intron | SCLC-P |
| chr1: 15094806 | ENST00000636203.1 | KAZN | Intron | SCLC-P |
| chr1: 15094835 | ENST00000636203.1 | KAZN | Exon | SCLC-P |
| chr1: 15094881 | ENST00000636203.1 | KAZN | Exon | SCLC-P |
| chr1: 15094890 | ENST00000636203.1 | KAZN | Exon | SCLC-P |
| chr1: 15094901 | ENST00000636203.1 | KAZN | Exon | SCLC-P |
| chr1: 15094919 | ENST00000636203.1 | KAZN | Exon | SCLC-P |
| chr1: 15094947 | ENST00000636203.1 | KAZN | Intron | SCLC-P |
| chr1: 15094956 | ENST00000636203.1 | KAZN | Intron | SCLC-P |
| chr1: 15094957 | ENST00000636203.1 | KAZN | Intron | SCLC-P |
| chr1: 15095070 | ENST00000636203.1 | KAZN | Intron | SCLC-P |
| chr1: 15095198 | ENST00000636203.1 | KAZN | Intron | SCLC-P |
| chr1: 15095209 | ENST00000636203.1 | KAZN | Intron | SCLC-P |
| chr1: 15095236 | ENST00000636203.1 | KAZN | Intron | SCLC-P |
| chr1: 15170295 | ENST00000428417.5 | TMEM51 | Intron | SCLC-P |
| chr1: 15176385 | ENST00000428417.5 | TMEM51 | Intron | SCLC-P |
| chr1: 15176487 | ENST00000428417.5 | TMEM51 | Intron | SCLC-P |
| chr1: 15207320 | ENST00000428417.5 | TMEM51 | Intron | SCLC-P |
| chr1: 15207443 | ENST00000428417.5 | TMEM51 | Intron | SCLC-P |
| chr1: 15207597 | ENST00000428417.5 | TMEM51 | Intron | SCLC-P |
| chr1: 15207633 | ENST00000428417.5 | TMEM51 | Intron | SCLC-P |
| chr1: 15207848 | ENST00000428417.5 | TMEM51 | Intron | SCLC-P |
| chr1: 15284221 | ENST00000433640.6 | FHAD1 | Intron | SCLC-P |
| chr1: 15284246 | ENST00000433640.6 | FHAD1 | Intron | SCLC-P |
| chr1: 15284247 | ENST00000433640.6 | FHAD1 | Intron | SCLC-P |
| chr1: 15284251 | ENST00000433640.6 | FHAD1 | Intron | SCLC-P |
| chr1: 15284252 | ENST00000433640.6 | FHAD1 | Intron | SCLC-P |
| chr1: 15284256 | ENST00000433640.6 | FHAD1 | Intron | SCLC-P |
| chr1: 15284262 | ENST00000433640.6 | FHAD1 | Intron | SCLC-P |
| chr1: 15284296 | ENST00000433640.6 | FHAD1 | Intron | SCLC-P |
| chr1: 15284297 | ENST00000433640.6 | FHAD1 | Intron | SCLC-P |
| chr1: 15314484 | ENST00000524761.5 | FHAD1 | Intron | SCLC-P |
| chr1: 15378800 | ENST00000358897.8 | FHAD1 | Intron | SCLC-P |
| chr1: 15378835 | ENST00000358897.8 | FHAD1 | Intron | SCLC-P |
| chr1: 15467428 | ENST00000359621.5 | CELA2A | Exon | SCLC-P |
| chr1: 15467442 | ENST00000359621.5 | CELA2A | Exon | SCLC-P |
| chr1: 15467448 | ENST00000359621.5 | CELA2A | Exon | SCLC-P |
| chr1: 15467464 | ENST00000359621.5 | CELA2A | Exon | SCLC-P |
| chr1: 15467505 | ENST00000359621.5 | CELA2A | Exon | SCLC-P |
| chr1: 15467507 | ENST00000359621.5 | CELA2A | Exon | SCLC-P |
| chr1: 15467538 | ENST00000359621.5 | CELA2A | Exon | SCLC-P |
| chr1: 15467548 | ENST00000494280.1 | CELA2B | Intron | SCLC-P |
| chr1: 15619223 | ENST00000480945.6 | DDI2 | Intron | SCLC-P |
| chr1: 15619262 | ENST00000480945.6 | DDI2 | Intron | SCLC-P |
| chr1: 15619265 | ENST00000480945.6 | DDI2 | Intron | SCLC-P |
| chr1: 15619266 | ENST00000480945.6 | DDI2 | Intron | SCLC-P |
| chr1: 15619267 | ENST00000480945.6 | DDI2 | Intron | SCLC-P |
| chr1: 15619268 | ENST00000480945.6 | DDI2 | Intron | SCLC-P |
| chr1: 15619269 | ENST00000480945.6 | DDI2 | Intron | SCLC-P |
| chr1: 15619270 | ENST00000480945.6 | DDI2 | Intron | SCLC-P |
| chr1: 15619278 | ENST00000480945.6 | DDI2 | Intron | SCLC-P |
| chr1: 15619372 | ENST00000480945.6 | DDI2 | Intron | SCLC-P |
| chr1: 15619373 | ENST00000480945.6 | DDI2 | Intron | SCLC-P |
| chr1: 15619377 | ENST00000480945.6 | DDI2 | Intron | SCLC-P |
| chr1: 15619378 | ENST00000480945.6 | DDI2 | Intron | SCLC-P |
| chr1: 15619395 | ENST00000480945.6 | DDI2 | Intron | SCLC-P |
| chr1: 15619396 | ENST00000480945.6 | DDI2 | Intron | SCLC-P |
| chr1: 15619415 | ENST00000480945.6 | DDI2 | Intron | SCLC-P |
| chr1: 15619416 | ENST00000480945.6 | DDI2 | Intron | SCLC-P |
| chr1: 15619421 | ENST00000480945.6 | DDI2 | Intron | SCLC-P |
| chr1: 15619422 | ENST00000480945.6 | DDI2 | Intron | SCLC-P |
| chr1: 15619483 | ENST00000480945.6 | DDI2 | Intron | SCLC-P |
| chr1: 15619484 | ENST00000480945.6 | DDI2 | Intron | SCLC-P |
| chr1: 15619488 | ENST00000480945.6 | DDI2 | Intron | SCLC-P |
| chr1: 15619489 | ENST00000480945.6 | DDI2 | Intron | SCLC-P |
| chr1: 15619500 | ENST00000480945.6 | DDI2 | Intron | SCLC-P |
| chr1: 15619501 | ENST00000480945.6 | DDI2 | Intron | SCLC-P |
| chr1: 15619522 | ENST00000480945.6 | DDI2 | Intron | SCLC-P |
| chr1: 15619524 | ENST00000480945.6 | DDI2 | Intron | SCLC-P |
| chr1: 15619525 | ENST00000480945.6 | DDI2 | Intron | SCLC-P |
| chr1: 15619530 | ENST00000480945.6 | DDI2 | Intron | SCLC-P |
| chr1: 15619531 | ENST00000480945.6 | DDI2 | Intron | SCLC-P |
| chr1: 15619554 | ENST00000480945.6 | DDI2 | Intron | SCLC-P |
| chr1: 15619555 | ENST00000480945.6 | DDI2 | Intron | SCLC-P |
| chr1: 15619560 | ENST00000480945.6 | DDI2 | Intron | SCLC-P |
| chr1: 15619561 | ENST00000480945.6 | DDI2 | Intron | SCLC-P |
| chr1: 15619587 | ENST00000480945.6 | DDI2 | Intron | SCLC-P |
| chr1: 15619697 | ENST00000480945.6 | DDI2 | Intron | SCLC-P |
| chr1: 15619708 | ENST00000480945.6 | DDI2 | Intron | SCLC-P |
| chr1: 15619713 | ENST00000480945.6 | DDI2 | Intron | SCLC-P |
| chr1: 15619739 | ENST00000480945.6 | DDI2 | Intron | SCLC-P |
| chr1: 15619745 | ENST00000480945.6 | DDI2 | Intron | SCLC-P |
| chr1: 15974871 | ENST00000375743.8 | ZBTB17 | Intron | SCLC-P |
| chr1: 16360965 | ENST00000475078.1 | SZRD1 | Intron | SCLC-P |
| chr1: 16612139 | ENST00000430580.6 | NBPF1 | Intron | SCLC-P |
| chr1: 16612246 | ENST00000430580.6 | NBPF1 | Intron | SCLC-P |
| chr1: 16612282 | ENST00000430580.6 | NBPF1 | Intron | SCLC-P |
| chr1: 16612286 | ENST00000430580.6 | NBPF1 | Intron | SCLC-P |
| chr1: 17008777 | ENST00000341676.9 | ATP13A2 | Intron | SCLC-P |
| chr1: 17008821 | ENST00000341676.9 | ATP13A2 | Intron | SCLC-P |
| chr1: 17413855 | ENST00000375436.9 | RCC2 | Intron | SCLC-P |
| chr1: 17427886 | ENST00000375436.9 | RCC2 | Intron | SCLC-P |
| chr1: 17427912 | ENST00000375436.9 | RCC2 | Intron | SCLC-P |
| chr1: 17427919 | ENST00000375436.9 | RCC2 | Intron | SCLC-P |
| chr1: 18252569 | ENST00000251296.4 | IGSF21 | Intron | SCLC-P |
| chr1: 18252756 | ENST00000251296.4 | IGSF21 | Intron | SCLC-P |
| chr1: 18252757 | ENST00000251296.4 | IGSF21 | Intron | SCLC-P |
| chr1: 18252764 | ENST00000251296.4 | IGSF21 | Intron | SCLC-P |
| chr1: 18262355 | ENST00000251296.4 | IGSF21 | Intron | SCLC-P |
| chr1: 18268619 | ENST00000251296.4 | IGSF21 | Intron | SCLC-P |
| chr1: 18269952 | ENST00000251296.4 | IGSF21 | Intron | SCLC-P |
| chr1: 18270018 | ENST00000251296.4 | IGSF21 | Intron | SCLC-P |
| chr1: 18282229 | ENST00000251296.4 | IGSF21 | Intron | SCLC-P |
| chr1: 18291342 | ENST00000251296.4 | IGSF21 | Intron | SCLC-P |
| chr1: 18291456 | ENST00000251296.4 | IGSF21 | Intron | SCLC-P |
| chr1: 18296112 | ENST00000251296.4 | IGSF21 | Intron | SCLC-P |
| chr1: 18925512 | ENST00000455833.7 | IFFO2 | Intron | SCLC-P |
| chr1: 19366460 | ENST00000264202.8 | CAPZB | Intron | SCLC-P |
| chr1: 19477027 | ENST00000459967.6 | CAPZB | Intron | SCLC-P |
| chr1: 19920429 | ENST00000375116.3 | PLA2G2E | Exon | SCLC-P |
| chr1: 19920447 | ENST00000375116.3 | PLA2G2E | Exon | SCLC-P |
| chr1: 19920448 | ENST00000375116.3 | PLA2G2E | Exon | SCLC-P |
| chr1: 19920690 | ENST00000375116.3 | PLA2G2E | Intron | SCLC-P |
| chr1: 19922634 | ENST00000375116.3 | PLA2G2E | Exon | SCLC-P |
| chr1: 19922651 | ENST00000375116.3 | PLA2G2E | Exon | SCLC-P |
| chr1: 19922657 | ENST00000375116.3 | PLA2G2E | Exon | SCLC-P |
| chr1: 19922712 | ENST00000375116.3 | PLA2G2E | Exon | SCLC-P |
| chr1: 20165043 | ENST00000495760.2 | PLA2G2C | Intron | SCLC-P |
| chr1: 20336453 | ENST00000485375.6 | VWA5B1 | Exon | SCLC-P |
| chr1: 20721988 | ENST00000444387.7 | SH2D5 | Exon | SCLC-P |
| chr1: 21547931 | ENST00000468526.1 | ALPL | Intron | SCLC-P |
| chr1: 21548030 | ENST00000468526.1 | ALPL | Intron | SCLC-P |
| chr1: 21548121 | ENST00000468526.1 | ALPL | Intron | SCLC-P |
| chr1: 21597784 | ENST00000290101.8 | RAP1GAP | Intron | SCLC-P |
| chr1: 21603872 | ENST00000611549.4 | RAP1GAP | Exon | SCLC-P |
| chr1: 21619318 | ENST00000290101.8 | RAP1GAP | Intron | SCLC-P |
| chr1: 21627288 | ENST00000290101.8 | RAP1GAP | Intron | SCLC-P |
| chr1: 21664057 | ENST00000290101.8 | RAP1GAP | Intron | SCLC-P |
| chr1: 21664061 | ENST00000290101.8 | RAP1GAP | Intron | SCLC-P |
| chr1: 22589212 | ENST00000374644.8 | EPHA8 | Exon | SCLC-P |
| chr1: 22591874 | ENST00000166244.8 | EPHA8 | Intron | SCLC-P |
| chr1: 22597780 | ENST00000166244.8 | EPHA8 | Exon | SCLC-P |
| chr1: 22597794 | ENST00000166244.8 | EPHA8 | Exon | SCLC-P |
| chr1: 22597799 | ENST00000166244.8 | EPHA8 | Exon | SCLC-P |
| chr1: 22598934 | ENST00000166244.8 | EPHA8 | Exon | SCLC-P |
| chr1: 22601202 | ENST00000166244.8 | EPHA8 | Intron | SCLC-P |
| chr1: 22601203 | ENST00000166244.8 | EPHA8 | Intron | SCLC-P |
| chr1: 22643723 | ENST00000374639.7 | C1QC | UTR5 | SCLC-P |
| chr1: 22643769 | ENST00000374637.1 | C1QC | UTR5 | SCLC-P |
| chr1: 22739946 | ENST00000544305.5 | EPHB2 | Intron | SCLC-P |
| chr1: 22963336 | ENST00000618559.1 | LACTBL1 | Promoter | SCLC-P |
| chr1: 24385760 | ENST00000003583.12 | STPG1 | Intron | SCLC-P |
| chr1: 24744410 | ENST00000497755.1 | CLIC4 | Promoter | SCLC-P |
| chr1: 24744411 | ENST00000497755.1 | CLIC4 | Promoter | SCLC-P |
| chr1: 24744414 | ENST00000497755.1 | CLIC4 | Promoter | SCLC-P |
| chr1: 24744415 | ENST00000497755.1 | CLIC4 | Promoter | SCLC-P |
| chr1: 24744417 | ENST00000497755.1 | CLIC4 | Promoter | SCLC-P |
| chr1: 24744419 | ENST00000497755.1 | CLIC4 | Promoter | SCLC-P |
| chr1: 24744429 | ENST00000497755.1 | CLIC4 | Promoter | SCLC-P |
| chr1: 24744466 | ENST00000497755.1 | CLIC4 | Promoter | SCLC-P |
| chr1: 25809315 | ENST00000354177.8 | SELENON | Intron | SCLC-P |
| chr1: 25809316 | ENST00000354177.8 | SELENON | Intron | SCLC-P |
| chr1: 25809343 | ENST00000354177.8 | SELENON | Intron | SCLC-P |
| chr1: 28583630 | ENST00000488745.5 | SNHG12 | Promoter | SCLC-P |
| chr1: 28583631 | ENST00000488745.5 | SNHG12 | Promoter | SCLC-P |
| chr1: 28583644 | ENST00000488745.5 | SNHG12 | Promoter | SCLC-P |
| chr1: 28583656 | ENST00000488745.5 | SNHG12 | Promoter | SCLC-P |
| chr1: 28583660 | ENST00000488745.5 | SNHG12 | Promoter | SCLC-P |
| chr1: 28583661 | ENST00000488745.5 | SNHG12 | Promoter | SCLC-P |
| chr1: 28583690 | ENST00000488745.5 | SNHG12 | Promoter | SCLC-P |
| chr1: 28583698 | ENST00000488745.5 | SNHG12 | Promoter | SCLC-P |
| chr1: 28583700 | ENST00000488745.5 | SNHG12 | Promoter | SCLC-P |
| chr1: 28583701 | ENST00000488745.5 | SNHG12 | Promoter | SCLC-P |
| chr1: 28583705 | ENST00000488745.5 | SNHG12 | Promoter | SCLC-P |
| chr1: 28715324 | ENST00000373816.5 | GMEB1 | Exon | SCLC-P |
| chr1: 28715336 | ENST00000373816.5 | GMEB1 | Exon | SCLC-P |
| chr1: 28715337 | ENST00000373816.5 | GMEB1 | Exon | SCLC-P |
| chr1: 28715364 | ENST00000373816.5 | GMEB1 | Exon | SCLC-P |
| chr1: 28715380 | ENST00000373816.5 | GMEB1 | Exon | SCLC-P |
| chr1: 31669817 | ENST00000468459.5 | COL16A1 | Intron | SCLC-P |
| chr1: 31669823 | ENST00000468459.5 | COL16A1 | Intron | SCLC-P |
| chr1: 31669841 | ENST00000468459.5 | COL16A1 | Intron | SCLC-P |
| chr1: 32674128 | ENST00000373485.5 | RBBP4 | Intron | SCLC-P |
| chr1: 33625012 | ENST00000373381.8 | CSMD2 | Intron | SCLC-P |
| chr1: 33625019 | ENST00000373381.8 | CSMD2 | Intron | SCLC-P |
| chr1: 33625030 | ENST00000373381.8 | CSMD2 | Intron | SCLC-P |
| chr1: 33625034 | ENST00000373381.8 | CSMD2 | Intron | SCLC-P |
| chr1: 33625058 | ENST00000373377.1 | CSMD2 | UTR5 | SCLC-P |
| chr1: 33625064 | ENST00000373377.1 | CSMD2 | UTR5 | SCLC-P |
| chr1: 34153164 | ENST00000373388.6 | CSMD2 | Intron | SCLC-P |
| chr1: 34887201 | ENST00000235180.4 | DLGAP3 | Intron | SCLC-P |
| chr1: 34887202 | ENST00000235180.4 | DLGAP3 | Intron | SCLC-P |
| chr1: 34887206 | ENST00000235180.4 | DLGAP3 | Intron | SCLC-P |
| chr1: 34887207 | ENST00000235180.4 | DLGAP3 | Intron | SCLC-P |
| chr1: 34887234 | ENST00000235180.4 | DLGAP3 | Intron | SCLC-P |
| chr1: 34887235 | ENST00000235180.4 | DLGAP3 | Intron | SCLC-P |
| chr1: 34887244 | ENST00000235180.4 | DLGAP3 | Intron | SCLC-P |
| chr1: 34887245 | ENST00000235180.4 | DLGAP3 | Intron | SCLC-P |
| chr1: 34904362 | ENST00000373347.6 | DLGAP3 | Exon | SCLC-P |
| chr1: 34904494 | ENST00000373347.6 | DLGAP3 | Exon | SCLC-P |
| chr1: 35560570 | ENST00000373253.7 | NCDN | Exon | SCLC-P |
| chr1: 36867899 | ENST00000373091.8 | GRIK3 | Intron | SCLC-P |
| chr1: 36868049 | ENST00000373091.8 | GRIK3 | Intron | SCLC-P |
| chr1: 36966667 | ENST00000373091.8 | GRIK3 | Intron | SCLC-P |
| chr1: 36966850 | ENST00000373091.8 | GRIK3 | Intron | SCLC-P |
| chr1: 37448607 | ENST00000668045.1 | LINC01137 | Intron | SCLC-P |
| chr1: 37452230 | ENST00000668045.1 | LINC01137 | Intron | SCLC-P |
| chr1: 37452442 | ENST00000668045.1 | LINC01137 | Intron | SCLC-P |
| chr1: 37475662 | ENST00000640233.1 | ZC3H12A | Exon | SCLC-P |
| chr1: 37475697 | ENST00000640233.1 | ZC3H12A | Exon | SCLC-P |
| chr1: 37475773 | ENST00000640233.1 | ZC3H12A | Exon | SCLC-P |
| chr1: 37475777 | ENST00000640233.1 | ZC3H12A | Exon | SCLC-P |
| chr1: 37475783 | ENST00000640233.1 | ZC3H12A | Exon | SCLC-P |
| chr1: 37475796 | ENST00000640233.1 | ZC3H12A | Exon | SCLC-P |
| chr1: 37719021 | ENST00000534097.5 | EPHA10 | Intron | SCLC-P |
| chr1: 37719040 | ENST00000534097.5 | EPHA10 | Intron | SCLC-P |
| chr1: 37719045 | ENST00000534097.5 | EPHA10 | Intron | SCLC-P |
| chr1: 37719046 | ENST00000534097.5 | EPHA10 | Intron | SCLC-P |
| chr1: 37736214 | ENST00000446149.6 | EPHA10 | Intron | SCLC-P |
| chr1: 38052629 | ENST00000432922.5 | NA | Intron | SCLC-P |
| chr1: 38052644 | ENST00000432922.5 | NA | Intron | SCLC-P |
| chr1: 38052658 | ENST00000432922.5 | NA | Intron | SCLC-P |
| chr1: 38052759 | ENST00000432922.5 | NA | Intron | SCLC-P |
| chr1: 38062291 | ENST00000432922.5 | NA | Intron | SCLC-P |
| chr1: 38086104 | ENST00000432922.5 | NA | Intron | SCLC-P |
| chr1: 38136298 | ENST00000641212.1 | NA | Exon | SCLC-P |
| chr1: 38137909 | ENST00000641212.1 | NA | Intron | SCLC-P |
| chr1: 38137950 | ENST00000641212.1 | NA | Intron | SCLC-P |
| chr1: 40770819 | ENST00000414185.1 | NFYC | Intron | SCLC-P |
| chr1: 40770919 | ENST00000414185.1 | NFYC | Intron | SCLC-P |
| chr1: 42887980 | ENST00000448759.1 | NA | Exon | SCLC-P |
| chr1: 42887991 | ENST00000448759.1 | NA | Exon | SCLC-P |
| chr1: 45444036 | ENST00000671898.1 | MUTYH | Intron | SCLC-P |
| chr1: 45583057 | ENST00000437901.6 | NASP | Promoter | SCLC-P |
| chr1: 45622828 | ENST00000445048.2 | CCDC17 | Intron | SCLC-P |
| chr1: 45622851 | ENST00000445048.2 | CCDC17 | Intron | SCLC-P |
| chr1: 45622853 | ENST00000445048.2 | CCDC17 | Intron | SCLC-P |
| chr1: 45622870 | ENST00000445048.2 | CCDC17 | Intron | SCLC-P |
| chr1: 45622929 | ENST00000445048.2 | CCDC17 | Intron | SCLC-P |
| chr1: 45622937 | ENST00000445048.2 | CCDC17 | Intron | SCLC-P |
| chr1: 45622945 | ENST00000445048.2 | CCDC17 | Intron | SCLC-P |
| chr1: 45622963 | ENST00000445048.2 | CCDC17 | Intron | SCLC-P |
| chr1: 45622983 | ENST00000445048.2 | CCDC17 | Intron | SCLC-P |
| chr1: 45623010 | ENST00000445048.2 | CCDC17 | Intron | SCLC-P |
| chr1: 45623011 | ENST00000445048.2 | CCDC17 | Intron | SCLC-P |
| chr1: 45623012 | ENST00000445048.2 | CCDC17 | Intron | SCLC-P |
| chr1: 45623019 | ENST00000445048.2 | CCDC17 | Intron | SCLC-P |
| chr1: 45623020 | ENST00000445048.2 | CCDC17 | Intron | SCLC-P |
| chr1: 45623058 | ENST00000445048.2 | CCDC17 | Intron | SCLC-P |
| chr1: 45623059 | ENST00000445048.2 | CCDC17 | Intron | SCLC-P |
| chr1: 45623066 | ENST00000445048.2 | CCDC17 | Intron | SCLC-P |
| chr1: 45623067 | ENST00000445048.2 | CCDC17 | Intron | SCLC-P |
| chr1: 45623076 | ENST00000445048.2 | CCDC17 | Intron | SCLC-P |
| chr1: 45623077 | ENST00000445048.2 | CCDC17 | Intron | SCLC-P |
| chr1: 45623081 | ENST00000445048.2 | CCDC17 | Intron | SCLC-P |
| chr1: 45623082 | ENST00000445048.2 | CCDC17 | Intron | SCLC-P |
| chr1: 45623083 | ENST00000445048.2 | CCDC17 | Intron | SCLC-P |
| chr1: 45623084 | ENST00000445048.2 | CCDC17 | Intron | SCLC-P |
| chr1: 45623105 | ENST00000445048.2 | CCDC17 | Intron | SCLC-P |
| chr1: 45623106 | ENST00000445048.2 | CCDC17 | Intron | SCLC-P |
| chr1: 45623108 | ENST00000445048.2 | CCDC17 | Intron | SCLC-P |
| chr1: 45623109 | ENST00000445048.2 | CCDC17 | Intron | SCLC-P |
| chr1: 45623129 | ENST00000445048.2 | CCDC17 | Intron | SCLC-P |
| chr1: 45623130 | ENST00000445048.2 | CCDC17 | Intron | SCLC-P |
| chr1: 45623134 | ENST00000445048.2 | CCDC17 | Intron | SCLC-P |
| chr1: 45623145 | ENST00000445048.2 | CCDC17 | Intron | SCLC-P |
| chr1: 45623150 | ENST00000445048.2 | CCDC17 | Intron | SCLC-P |
| chr1: 45623151 | ENST00000445048.2 | CCDC17 | Intron | SCLC-P |
| chr1: 45623169 | ENST00000445048.2 | CCDC17 | Intron | SCLC-P |
| chr1: 45623172 | ENST00000445048.2 | CCDC17 | Intron | SCLC-P |
| chr1: 45623199 | ENST00000445048.2 | CCDC17 | Intron | SCLC-P |
| chr1: 45623221 | ENST00000445048.2 | CCDC17 | Intron | SCLC-P |
| chr1: 45623224 | ENST00000445048.2 | CCDC17 | Intron | SCLC-P |
| chr1: 45623226 | ENST00000445048.2 | CCDC17 | Intron | SCLC-P |
| chr1: 45623241 | ENST00000445048.2 | CCDC17 | Intron | SCLC-P |
| chr1: 47439324 | ENST00000334793.5 | FOXD2 | Exon | SCLC-P |
| chr1: 47439345 | ENST00000334793.5 | FOXD2 | Exon | SCLC-P |
| chr1: 47439462 | ENST00000334793.5 | FOXD2 | Exon | SCLC-P |
| chr1: 47439464 | ENST00000334793.5 | FOXD2 | Exon | SCLC-P |
| chr1: 47439522 | ENST00000334793.5 | FOXD2 | Exon | SCLC-P |
| chr1: 47887584 | ENST00000606738.3 | TRABD2B | Intron | SCLC-P |
| chr1: 47924043 | ENST00000606738.3 | TRABD2B | Intron | SCLC-P |
| chr1: 47924219 | ENST00000606738.3 | TRABD2B | Intron | SCLC-P |
| chr1: 48355580 | ENST00000371843.7 | SPATA6 | Intron | SCLC-P |
| chr1: 50309008 | ENST00000641180.1 | NA | Intron | SCLC-P |
| chr1: 50309014 | ENST00000641180.1 | NA | Intron | SCLC-P |
| chr1: 53077723 | ENST00000447867.1 | NA | Intron | SCLC-P |
| chr1: 53077739 | ENST00000447867.1 | NA | Intron | SCLC-P |
| chr1: 53077838 | ENST00000447867.1 | NA | Intron | SCLC-P |
| chr1: 53090927 | ENST00000620288.4 | SLC1A7 | Intron | SCLC-P |
| chr1: 53836270 | ENST00000480952.1 | NDC1 | Intron | SCLC-P |
| chr1: 53836274 | ENST00000480952.1 | NDC1 | Intron | SCLC-P |
| chr1: 53836425 | ENST00000480952.1 | NDC1 | Intron | SCLC-P |
| chr1: 53836589 | ENST00000480952.1 | NDC1 | Intron | SCLC-P |
| chr1: 59712748 | ENST00000476939.5 | FGGY | Intron | SCLC-P |
| chr1: 62194337 | ENST00000498273.2 | L1TD1 | Promoter | SCLC-P |
| chr1: 64852033 | ENST00000342505.5 | JAK1 | Intron | SCLC-P |
| chr1: 64852053 | ENST00000342505.5 | JAK1 | Intron | SCLC-P |
| chr1: 95228039 | ENST00000604534.5 | TLCD4-RWDD3 | Intron | SCLC-P |
| chr1: 109294770 | ENST00000489706.5 | MYBPHL | Intron | SCLC-P |
| chr1: 110518605 | ENST00000369771.4 | KCNA10 | Exon | SCLC-P |
| chr1: 110530454 | ENST00000640680.1 | KCNA2 | Intron | SCLC-P |
| chr1: 110530473 | ENST00000640680.1 | KCNA2 | Intron | SCLC-P |
| chr1: 111900287 | ENST00000369697.5 | KCND3 | Intron | SCLC-P |
| chr1: 113906885 | ENST00000648795.1 | DCLRE1B | Intron | SCLC-P |
| chr1: 116668193 | ENST00000369486.7 | IGSF3 | Promoter | SCLC-P |
| chr1: 118985198 | ENST00000207157.7 | TBX15 | Intron | SCLC-P |
| chr1: 118989370 | ENST00000207157.7 | TBX15 | UTR5 | SCLC-P |
| chr1: 118989427 | ENST00000207157.7 | TBX15 | UTR5 | SCLC-P |
| chr1: 118989611 | ENST00000207157.7 | TBX15 | Promoter | SCLC-P |
| chr1: 118989698 | ENST00000207157.7 | TBX15 | Promoter | SCLC-P |
| chr1: 118990071 | ENST00000207157.7 | TBX15 | Promoter | SCLC-P |
| chr1: 118990113 | ENST00000207157.7 | TBX15 | Promoter | SCLC-P |
| chr1: 118990150 | ENST00000207157.7 | TBX15 | Promoter | SCLC-P |
| chr1: 118990209 | ENST00000207157.7 | TBX15 | Promoter | SCLC-P |
| chr1: 118990303 | ENST00000207157.7 | TBX15 | Promoter | SCLC-P |
| chr1: 119623849 | ENST00000421812.3 | ZNF697 | Exon | SCLC-P |
| chr1: 119623858 | ENST00000421812.3 | ZNF697 | Exon | SCLC-P |
| chr1: 119623861 | ENST00000421812.3 | ZNF697 | Exon | SCLC-P |
| chr1: 119623868 | ENST00000421812.3 | ZNF697 | Exon | SCLC-P |
| chr1: 119623882 | ENST00000421812.3 | ZNF697 | Exon | SCLC-P |
| chr1: 119623888 | ENST00000421812.3 | ZNF697 | Exon | SCLC-P |
| chr1: 119623889 | ENST00000421812.3 | ZNF697 | Exon | SCLC-P |
| chr1: 119623910 | ENST00000421812.3 | ZNF697 | Exon | SCLC-P |
| chr1: 119623926 | ENST00000421812.3 | ZNF697 | Exon | SCLC-P |
| chr1: 119623936 | ENST00000421812.3 | ZNF697 | Exon | SCLC-P |
| chr1: 151133216 | ENST00000368912.7 | SEMA6C | Exon | SCLC-P |
| chr1: 151133246 | ENST00000368912.7 | SEMA6C | Exon | SCLC-P |
| chr1: 151133249 | ENST00000368912.7 | SEMA6C | Exon | SCLC-P |
| chr1: 151133258 | ENST00000368912.7 | SEMA6C | Exon | SCLC-P |
| chr1: 151133260 | ENST00000368912.7 | SEMA6C | Exon | SCLC-P |
| chr1: 151133266 | ENST00000368912.7 | SEMA6C | Exon | SCLC-P |
| chr1: 151133385 | ENST00000368912.7 | SEMA6C | Exon | SCLC-P |
| chr1: 154503013 | ENST00000368482.8 | TDRD10 | UTR5 | SCLC-P |
| chr1: 154503020 | ENST00000368482.8 | TDRD10 | UTR5 | SCLC-P |
| chr1: 154503022 | ENST00000368482.8 | TDRD10 | UTR5 | SCLC-P |
| chr1: 154503027 | ENST00000368482.8 | TDRD10 | UTR5 | SCLC-P |
| chr1: 156076881 | ENST00000532414.3 | MEX3A | Exon | SCLC-P |
| chr1: 156076891 | ENST00000532414.3 | MEX3A | Exon | SCLC-P |
| chr1: 161152335 | ENST00000467540.5 | UFC1 | Promoter | SCLC-P |
| chr1: 161152367 | ENST00000467540.5 | UFC1 | Promoter | SCLC-P |
| chr1: 161152395 | ENST00000467540.5 | UFC1 | Promoter | SCLC-P |
| chr1: 161152405 | ENST00000467540.5 | UFC1 | Promoter | SCLC-P |
| chr1: 161152410 | ENST00000467540.5 | UFC1 | Promoter | SCLC-P |
| chr1: 161152581 | ENST00000467540.5 | UFC1 | Promoter | SCLC-P |
| chr1: 161152593 | ENST00000467540.5 | UFC1 | Promoter | SCLC-P |
| chr1: 161152597 | ENST00000467540.5 | UFC1 | Promoter | SCLC-P |
| chr1: 161152734 | ENST00000467540.5 | UFC1 | Promoter | SCLC-P |
| chr1: 161152774 | ENST00000467540.5 | UFC1 | Promoter | SCLC-P |
| chr1: 161152776 | ENST00000467540.5 | UFC1 | Exon | SCLC-P |
| chr1: 161152777 | ENST00000467540.5 | UFC1 | Exon | SCLC-P |
| chr1: 161152778 | ENST00000467540.5 | UFC1 | Exon | SCLC-P |
| chr1: 161152779 | ENST00000467540.5 | UFC1 | Exon | SCLC-P |
| chr1: 161152788 | ENST00000467540.5 | UFC1 | Exon | SCLC-P |
| chr1: 161152789 | ENST00000467540.5 | UFC1 | Exon | SCLC-P |
| chr1: 161152871 | ENST00000467540.5 | UFC1 | Exon | SCLC-P |
| chr1: 161152872 | ENST00000467540.5 | UFC1 | Exon | SCLC-P |

TABLE 23
Additional methylation sites associated with the SCLC-I subtype

| Methylation Site | Transcript_ID | Gene_Symbol | Gene_Region | Subtype |
|---|---|---|---|---|
| chr1: 1212968 | ENST00000497869.5 | TNFRSF4 | Intron | SCLC-I |
| chr1: 3161157 | ENST00000511072.5 | PRDM16 | Intron | SCLC-I |
| chr1: 5994737 | ENST00000655548.1 | KCNAB2 | Intron | SCLC-I |
| chr1: 5994749 | ENST00000655548.1 | KCNAB2 | Intron | SCLC-I |
| chr1: 5994769 | ENST00000655548.1 | KCNAB2 | Intron | SCLC-I |
| chr1: 5994808 | ENST00000655548.1 | KCNAB2 | Intron | SCLC-I |
| chr1: 5994858 | ENST00000655548.1 | KCNAB2 | Intron | SCLC-I |
| chr1: 6455268 | ENST00000645284.1 | ESPN | Intron | SCLC-I |
| chr1: 6455428 | ENST00000645284.1 | ESPN | Intron | SCLC-I |
| chr1: 6455431 | ENST00000645284.1 | ESPN | Intron | SCLC-I |
| chr1: 6455441 | ENST00000645284.1 | ESPN | Intron | SCLC-I |
| chr1: 6455456 | ENST00000645284.1 | ESPN | Intron | SCLC-I |
| chr1: 6455459 | ENST00000645284.1 | ESPN | Intron | SCLC-I |
| chr1: 6455463 | ENST00000645284.1 | ESPN | Intron | SCLC-I |
| chr1: 6455469 | ENST00000645284.1 | ESPN | Intron | SCLC-I |
| chr1: 6455487 | ENST00000645284.1 | ESPN | Intron | SCLC-I |
| chr1: 6455494 | ENST00000645284.1 | ESPN | Intron | SCLC-I |
| chr1: 6455520 | ENST00000645284.1 | ESPN | Intron | SCLC-I |
| chr1: 6455535 | ENST00000645284.1 | ESPN | Intron | SCLC-I |
| chr1: 6455537 | ENST00000645284.1 | ESPN | Intron | SCLC-I |
| chr1: 7583360 | ENST00000303635.12 | CAMTA1 | Intron | SCLC-I |
| chr1: 8395198 | ENST00000337907.7 | RERE | Intron | SCLC-I |
| chr1: 8395241 | ENST00000337907.7 | RERE | Intron | SCLC-I |
| chr1: 8395242 | ENST00000337907.7 | RERE | Intron | SCLC-I |
| chr1: 8395246 | ENST00000337907.7 | RERE | Intron | SCLC-I |
| chr1: 8395332 | ENST00000337907.7 | RERE | Intron | SCLC-I |
| chr1: 9688018 | ENST00000377346.9 | PIK3CD | Intron | SCLC-I |
| chr1: 9937029 | ENST00000377223.6 | LZIC | Intron | SCLC-I |
| chr1: 10638826 | ENST00000377022.8 | CASZ1 | Exon | SCLC-I |
| chr1: 10638845 | ENST00000377022.8 | CASZ1 | Exon | SCLC-I |
| chr1: 10638850 | ENST00000377022.8 | CASZ1 | Exon | SCLC-I |
| chr1: 10638852 | ENST00000377022.8 | CASZ1 | Exon | SCLC-I |
| chr1: 10638871 | ENST00000377022.8 | CASZ1 | Exon | SCLC-I |
| chr1: 10638876 | ENST00000377022.8 | CASZ1 | Exon | SCLC-I |
| chr1: 10638880 | ENST00000377022.8 | CASZ1 | Exon | SCLC-I |
| chr1: 10638888 | ENST00000377022.8 | CASZ1 | Exon | SCLC-I |
| chr1: 10638897 | ENST00000377022.8 | CASZ1 | Exon | SCLC-I |
| chr1: 10638903 | ENST00000377022.8 | CASZ1 | Exon | SCLC-I |
| chr1: 10638909 | ENST00000377022.8 | CASZ1 | Exon | SCLC-I |
| chr1: 10638915 | ENST00000377022.8 | CASZ1 | Exon | SCLC-I |
| chr1: 10638918 | ENST00000377022.8 | CASZ1 | Exon | SCLC-I |
| chr1: 10638931 | ENST00000377022.8 | CASZ1 | Exon | SCLC-I |
| chr1: 10638937 | ENST00000377022.8 | CASZ1 | Exon | SCLC-I |
| chr1: 10638940 | ENST00000377022.8 | CASZ1 | Exon | SCLC-I |
| chr1: 10638948 | ENST00000377022.8 | CASZ1 | Exon | SCLC-I |
| chr1: 10638966 | ENST00000377022.8 | CASZ1 | Exon | SCLC-I |
| chr1: 10716488 | ENST00000377022.8 | CASZ1 | Intron | SCLC-I |
| chr1: 10716545 | ENST00000377022.8 | CASZ1 | Intron | SCLC-I |
| chr1: 10716580 | ENST00000377022.8 | CASZ1 | Intron | SCLC-I |
| chr1: 17691375 | ENST00000361221.8 | ARHGEF10L | Intron | SCLC-I |
| chr1: 17691405 | ENST00000361221.8 | ARHGEF10L | Intron | SCLC-I |
| chr1: 17691413 | ENST00000361221.8 | ARHGEF10L | Intron | SCLC-I |
| chr1: 17691414 | ENST00000361221.8 | ARHGEF10L | Intron | SCLC-I |
| chr1: 18951591 | ENST00000455833.7 | IFFO2 | Intron | SCLC-I |
| chr1: 18951738 | ENST00000455833.7 | IFFO2 | Intron | SCLC-I |
| chr1: 18951747 | ENST00000455833.7 | IFFO2 | Intron | SCLC-I |
| chr1: 19257381 | ENST00000479559.1 | MRTO4 | Promoter | SCLC-I |
| chr1: 20336464 | ENST00000485375.6 | VWA5B1 | Exon | SCLC-I |
| chr1: 20336485 | ENST00000485375.6 | VWA5B1 | Exon | SCLC-I |
| chr1: 20336496 | ENST00000485375.6 | VWA5B1 | Intron | SCLC-I |
| chr1: 20726105 | ENST00000460804.5 | SH2D5 | Intron | SCLC-I |
| chr1: 21293639 | ENST00000415912.6 | ECE1 | Intron | SCLC-I |
| chr1: 26319030 | ENST00000374213.3 | CD52 | Intron | SCLC-I |
| chr1: 26319037 | ENST00000374213.3 | CD52 | Intron | SCLC-I |
| chr1: 26319047 | ENST00000374213.3 | CD52 | Intron | SCLC-I |
| chr1: 26319074 | ENST00000374213.3 | CD52 | Intron | SCLC-I |
| chr1: 26319169 | ENST00000374213.3 | CD52 | Intron | SCLC-I |
| chr1: 26319170 | ENST00000374213.3 | CD52 | Intron | SCLC-I |
| chr1: 27349314 | ENST00000496001.5 | SYTL1 | Promoter | SCLC-I |
| chr1: 27349516 | ENST00000485269.1 | SYTL1 | Promoter | SCLC-I |
| chr1: 27349765 | ENST00000490170.5 | SYTL1 | Exon | SCLC-I |
| chr1: 27361001 | ENST00000357582.3 | MAP3K6 | Exon | SCLC-I |
| chr1: 27361110 | ENST00000357582.3 | MAP3K6 | Intron | SCLC-I |
| chr1: 43348635 | ENST00000638732.1 | MPL | Intron | SCLC-I |
| chr1: 43348644 | ENST00000638732.1 | MPL | Intron | SCLC-I |
| chr1: 43348678 | ENST00000638732.1 | MPL | Intron | SCLC-I |
| chr1: 43348748 | ENST00000638732.1 | MPL | Intron | SCLC-I |
| chr1: 43348752 | ENST00000638732.1 | MPL | Intron | SCLC-I |
| chr1: 43348792 | ENST00000638732.1 | MPL | Intron | SCLC-I |
| chr1: 43348803 | ENST00000638732.1 | MPL | Intron | SCLC-I |
| chr1: 43348811 | ENST00000638732.1 | MPL | Intron | SCLC-I |
| chr1: 43348817 | ENST00000638732.1 | MPL | Intron | SCLC-I |
| chr1: 87132531 | ENST00000370548.3 | LINC01140 | Intron | SCLC-I |
| chr1: 112621401 | ENST00000485542.5 | CAPZA1 | Intron | SCLC-I |
| chr1: 112621449 | ENST00000485542.5 | CAPZA1 | Intron | SCLC-I |
| chr1: 117151249 | ENST00000359008.8 | VTCN1 | Intron | SCLC-I |
| chr1: 118990162 | ENST00000207157.7 | TBX15 | Promoter | SCLC-I |
| chr1: 201648427 | ENST00000367302.5 | NAV1 | Intron | SCLC-I |
| chr1: 201648899 | ENST00000367296.8 | NAV1 | Exon | SCLC-I |
| chr1: 201648902 | ENST00000367296.8 | NAV1 | Exon | SCLC-I |
| chr1: 201648907 | ENST00000367296.8 | NAV1 | Exon | SCLC-I |
| chr1: 201648911 | ENST00000367296.8 | NAV1 | Exon | SCLC-I |
| chr1: 201648924 | ENST00000367296.8 | NAV1 | Exon | SCLC-I |
| chr1: 201649103 | ENST00000367296.8 | NAV1 | Exon | SCLC-I |
| chr1: 201649526 | ENST00000367302.5 | NAV1 | Intron | SCLC-I |
| chr1: 201649545 | ENST00000367302.5 | NAV1 | Intron | SCLC-I |
| chr1: 201649568 | ENST00000367302.5 | NAV1 | Intron | SCLC-I |
| chr1: 201649569 | ENST00000367302.5 | NAV1 | Intron | SCLC-I |
| chr1: 201649603 | ENST00000367302.5 | NAV1 | Intron | SCLC-I |
| chr1: 201649604 | ENST00000367302.5 | NAV1 | Intron | SCLC-I |
| chr1: 201649642 | ENST00000367302.5 | NAV1 | Intron | SCLC-I |
| chr1: 201649650 | ENST00000367302.5 | NAV1 | Intron | SCLC-I |
| chr1: 201650616 | ENST00000367302.5 | NAV1 | Intron | SCLC-I |
| chr1: 204194977 | ENST00000367194.5 | KISS1 | Intron | SCLC-I |
| chr1: 204379054 | ENST00000443515.1 | NA | Intron | SCLC-I |
| chr1: 212981403 | ENST00000366966.6 | VASH2 | Intron | SCLC-I |
| chr1: 212981607 | ENST00000366966.6 | VASH2 | Intron | SCLC-I |
| chr1: 226712073 | ENST00000272117.7 | ITPKB | Intron | SCLC-I |
| chr10: 6204251 | ENST00000379789.8 | PFKFB3 | Intron | SCLC-I |
| chr10: 11180532 | ENST00000633200.1 | CELF2 | Intron | SCLC-I |
| chr10: 21494147 | ENST00000658000.1 | MIR1915HG | Exon | SCLC-I |
| chr10: 21494162 | ENST00000658000.1 | MIR1915HG | Exon | SCLC-I |
| chr10: 21494163 | ENST00000658000.1 | MIR1915HG | Exon | SCLC-I |
| chr10: 48655690 | ENST00000460425.1 | ARHGAP22 | UTR5 | SCLC-I |
| chr10: 48655692 | ENST00000460425.1 | ARHGAP22 | UTR5 | SCLC-I |
| chr10: 48655695 | ENST00000460425.1 | ARHGAP22 | UTR5 | SCLC-I |
| chr10: 79261299 | ENST00000334512.10 | ZMIZ1 | Intron | SCLC-I |
| chr10: 79266615 | ENST00000334512.10 | ZMIZ1 | Intron | SCLC-I |
| chr10: 103664060 | ENST00000369774.8 | SH3PXD2A | Intron | SCLC-I |
| chr10: 103664157 | ENST00000369774.8 | SH3PXD2A | Intron | SCLC-I |
| chr10: 110072448 | ENST00000468251.5 | ADD3 | Intron | SCLC-I |
| chr10: 127738809 | ENST00000388920.5 | FOXI2 | Exon | SCLC-I |
| chr10: 127738825 | ENST00000388920.5 | FOXI2 | Exon | SCLC-I |
| chr10: 127738831 | ENST00000388920.5 | FOXI2 | Exon | SCLC-I |
| chr10: 127738832 | ENST00000388920.5 | FOXI2 | Exon | SCLC-I |
| chr10: 127738838 | ENST00000388920.5 | FOXI2 | Exon | SCLC-I |
| chr10: 127738839 | ENST00000388920.5 | FOXI2 | Exon | SCLC-I |
| chr10: 127738845 | ENST00000388920.5 | FOXI2 | Exon | SCLC-I |
| chr11: 1310191 | ENST00000530506.5 | TOLLIP | Promoter | SCLC-I |
| chr11: 1310193 | ENST00000530506.5 | TOLLIP | Promoter | SCLC-I |
| chr11: 1310196 | ENST00000530506.5 | TOLLIP | Promoter | SCLC-I |
| chr11: 1310201 | ENST00000530506.5 | TOLLIP | Promoter | SCLC-I |
| chr11: 1310212 | ENST00000530506.5 | TOLLIP | Promoter | SCLC-I |
| chr11: 1310227 | ENST00000530506.5 | TOLLIP | Promoter | SCLC-I |
| chr11: 2807477 | ENST00000155840.11 | KCNQ1 | Intron | SCLC-I |
| chr11: 2807560 | ENST00000155840.11 | KCNQ1 | Intron | SCLC-I |
| chr11: 44308565 | ENST00000652299.1 | ALX4 | Intron | SCLC-I |
| chr11: 62602888 | ENST00000460939.5 | EML3 | Exon | SCLC-I |
| chr11: 64082224 | ENST00000545464.5 | MACROD1 | Intron | SCLC-I |
| chr11: 64206312 | ENST00000544997.5 | FERMT3 | Promoter | SCLC-I |
| chr11: 64206975 | ENST00000544997.5 | FERMT3 | Intron | SCLC-I |
| chr11: 64206999 | ENST00000544997.5 | FERMT3 | Intron | SCLC-I |
| chr11: 64207068 | ENST00000544997.5 | FERMT3 | Intron | SCLC-I |
| chr11: 65640874 | ENST00000394224.3 | SIPA1 | Exon | SCLC-I |
| chr11: 65640888 | ENST00000394224.3 | SIPA1 | Exon | SCLC-I |
| chr11: 70424335 | ENST00000346329.7 | CTTN | Intron | SCLC-I |
| chr11: 70424342 | ENST00000346329.7 | CTTN | Intron | SCLC-I |
| chr11: 111414539 | ENST00000525890.1 | NA | Intron | SCLC-I |
| chr11: 113243216 | ENST00000620046.4 | NCAM1 | Intron | SCLC-I |
| chr11: 117992014 | ENST00000526544.5 | IL10RA | Intron | SCLC-I |
| chr11: 118088224 | ENST00000519126.1 | TMPRSS4 | Intron | SCLC-I |
| chr11: 118088319 | ENST00000519126.1 | TMPRSS4 | Intron | SCLC-I |
| chr11: 118088507 | ENST00000519126.1 | TMPRSS4 | Intron | SCLC-I |
| chr12: 1797594 | ENST00000541444.5 | CACNA2D4 | Exon | SCLC-I |
| chr12: 1834565 | ENST00000444595.6 | CACNA2D4 | Intron | SCLC-I |
| chr12: 1881455 | ENST00000382722.10 | CACNA2D4 | Intron | SCLC-I |
| chr12: 1921064 | ENST00000544163.1 | NA | Intron | SCLC-I |
| chr12: 1921078 | ENST00000544163.1 | NA | Intron | SCLC-I |
| chr12: 1921094 | ENST00000544163.1 | NA | Intron | SCLC-I |
| chr12: 2100317 | ENST00000543114.1 | CACNA1C | Intron | SCLC-I |
| chr12: 2100392 | ENST00000543114.1 | CACNA1C | Intron | SCLC-I |
| chr12: 2387345 | ENST00000327702.12 | CACNA1C | Intron | SCLC-I |
| chr12: 2387357 | ENST00000327702.12 | CACNA1C | Intron | SCLC-I |
| chr12: 2387399 | ENST00000327702.12 | CACNA1C | Intron | SCLC-I |
| chr12: 2412248 | ENST00000327702.12 | CACNA1C | Intron | SCLC-I |
| chr12: 2414570 | ENST00000327702.12 | CACNA1C | Intron | SCLC-I |
| chr12: 2578542 | ENST00000327702.12 | CACNA1C | Intron | SCLC-I |
| chr12: 2578571 | ENST00000327702.12 | CACNA1C | Intron | SCLC-I |
| chr12: 2578612 | ENST00000327702.12 | CACNA1C | Intron | SCLC-I |
| chr12: 2660687 | ENST00000327702.12 | CACNA1C | Intron | SCLC-I |
| chr12: 2660692 | ENST00000327702.12 | CACNA1C | Intron | SCLC-I |
| chr12: 2660693 | ENST00000327702.12 | CACNA1C | Intron | SCLC-I |
| chr12: 2660716 | ENST00000327702.12 | CACNA1C | Intron | SCLC-I |
| chr12: 6951500 | ENST00000534900.5 | PTPN6 | Intron | SCLC-I |
| chr12: 6952413 | ENST00000543744.1 | PTPN6 | Exon | SCLC-I |
| chr12: 6952423 | ENST00000543744.1 | PTPN6 | Exon | SCLC-I |
| chr12: 6959128 | ENST00000399448.5 | PTPN6 | Intron | SCLC-I |
| chr12: 6963341 | ENST00000384980.3 | MIR200C | Promoter | SCLC-I |
| chr12: 52250263 | ENST00000548657.5 | KRT7 | Intron | SCLC-I |
| chr12: 52250331 | ENST00000548657.5 | KRT7 | Intron | SCLC-I |
| chr12: 52250333 | ENST00000548657.5 | KRT7 | Intron | SCLC-I |
| chr12: 52250345 | ENST00000548657.5 | KRT7 | Intron | SCLC-I |
| chr12: 52250364 | ENST00000548657.5 | KRT7 | Intron | SCLC-I |
| chr12: 52250382 | ENST00000548657.5 | KRT7 | Intron | SCLC-I |
| chr12: 52250393 | ENST00000548657.5 | KRT7 | Intron | SCLC-I |
| chr12: 52258477 | ENST00000553310.6 | KRT86 | Intron | SCLC-I |
| chr12: 52258484 | ENST00000553310.6 | KRT86 | Intron | SCLC-I |
| chr12: 52258491 | ENST00000553310.6 | KRT86 | Intron | SCLC-I |
| chr12: 52258508 | ENST00000553310.6 | KRT86 | Intron | SCLC-I |
| chr12: 52258510 | ENST00000553310.6 | KRT86 | Intron | SCLC-I |
| chr12: 52258517 | ENST00000553310.6 | KRT86 | Intron | SCLC-I |
| chr12: 52258526 | ENST00000553310.6 | KRT86 | Intron | SCLC-I |
| chr12: 52258534 | ENST00000553310.6 | KRT86 | Intron | SCLC-I |
| chr12: 52258558 | ENST00000553310.6 | KRT86 | Intron | SCLC-I |
| chr12: 52258564 | ENST00000553310.6 | KRT86 | Intron | SCLC-I |
| chr12: 52258573 | ENST00000553310.6 | KRT86 | Intron | SCLC-I |
| chr12: 52258577 | ENST00000553310.6 | KRT86 | Intron | SCLC-I |
| chr12: 57225160 | ENST00000349394.6 | NXPH4 | Exon | SCLC-I |
| chr12: 57225395 | ENST00000349394.6 | NXPH4 | Exon | SCLC-I |
| chr12: 57235345 | ENST00000554503.5 | NDUFA4L2 | Exon | SCLC-I |
| chr12: 57726072 | ENST00000257897.7 | AGAP2 | Exon | SCLC-I |
| chr12: 57726090 | ENST00000257897.7 | AGAP2 | Exon | SCLC-I |
| chr12: 57726567 | ENST00000257897.7 | AGAP2 | Exon | SCLC-I |
| chr12: 57726572 | ENST00000257897.7 | AGAP2 | Exon | SCLC-I |
| chr12: 57726576 | ENST00000257897.7 | AGAP2 | Exon | SCLC-I |
| chr12: 57726578 | ENST00000257897.7 | AGAP2 | Exon | SCLC-I |
| chr12: 57726580 | ENST00000257897.7 | AGAP2 | Exon | SCLC-I |
| chr12: 57726587 | ENST00000257897.7 | AGAP2 | Exon | SCLC-I |
| chr12: 57726593 | ENST00000257897.7 | AGAP2 | Exon | SCLC-I |
| chr12: 57726596 | ENST00000257897.7 | AGAP2 | Exon | SCLC-I |
| chr12: 57726604 | ENST00000257897.7 | AGAP2 | Exon | SCLC-I |
| chr12: 57726607 | ENST00000257897.7 | AGAP2 | Exon | SCLC-I |
| chr12: 57726610 | ENST00000257897.7 | AGAP2 | Exon | SCLC-I |
| chr12: 57726621 | ENST00000257897.7 | AGAP2 | Exon | SCLC-I |
| chr12: 57726626 | ENST00000257897.7 | AGAP2 | Exon | SCLC-I |
| chr12: 57726639 | ENST00000257897.7 | AGAP2 | Exon | SCLC-I |
| chr12: 57726648 | ENST00000257897.7 | AGAP2 | Exon | SCLC-I |
| chr12: 57726650 | ENST00000257897.7 | AGAP2 | Exon | SCLC-I |
| chr12: 57726657 | ENST00000257897.7 | AGAP2 | Exon | SCLC-I |
| chr12: 57726666 | ENST00000257897.7 | AGAP2 | Exon | SCLC-I |
| chr12: 57726668 | ENST00000257897.7 | AGAP2 | Exon | SCLC-I |
| chr12: 57726674 | ENST00000257897.7 | AGAP2 | Exon | SCLC-I |
| chr12: 57726684 | ENST00000257897.7 | AGAP2 | Exon | SCLC-I |
| chr12: 57726692 | ENST00000257897.7 | AGAP2 | Exon | SCLC-I |
| chr12: 57726710 | ENST00000257897.7 | AGAP2 | Exon | SCLC-I |
| chr12: 57726713 | ENST00000257897.7 | AGAP2 | Exon | SCLC-I |
| chr12: 57726736 | ENST00000257897.7 | AGAP2 | Exon | SCLC-I |
| chr12: 57726740 | ENST00000257897.7 | AGAP2 | Exon | SCLC-I |
| chr12: 57726750 | ENST00000257897.7 | AGAP2 | Exon | SCLC-I |
| chr12: 57726753 | ENST00000257897.7 | AGAP2 | Exon | SCLC-I |
| chr12: 57726757 | ENST00000257897.7 | AGAP2 | Exon | SCLC-I |
| chr12: 57726767 | ENST00000257897.7 | AGAP2 | Exon | SCLC-I |
| chr12: 57726772 | ENST00000257897.7 | AGAP2 | Exon | SCLC-I |
| chr12: 57726782 | ENST00000257897.7 | AGAP2 | Exon | SCLC-I |
| chr12: 57726786 | ENST00000257897.7 | AGAP2 | Exon | SCLC-I |
| chr12: 57726788 | ENST00000257897.7 | AGAP2 | Exon | SCLC-I |
| chr12: 57726791 | ENST00000257897.7 | AGAP2 | Exon | SCLC-I |
| chr12: 57726803 | ENST00000542466.2 | AGAP2-AS1 | Intron | SCLC-I |
| chr12: 57726809 | ENST00000542466.2 | AGAP2-AS1 | Intron | SCLC-I |
| chr12: 57726815 | ENST00000542466.2 | AGAP2-AS1 | Intron | SCLC-I |
| chr12: 57726821 | ENST00000542466.2 | AGAP2-AS1 | Intron | SCLC-I |
| chr12: 57726823 | ENST00000542466.2 | AGAP2-AS1 | Intron | SCLC-I |
| chr12: 57726825 | ENST00000542466.2 | AGAP2-AS1 | Intron | SCLC-I |
| chr12: 57726836 | ENST00000542466.2 | AGAP2-AS1 | Intron | SCLC-I |
| chr12: 57736329 | ENST00000257897.7 | AGAP2 | Intron | SCLC-I |
| chr12: 57737351 | ENST00000257897.7 | AGAP2 | Intron | SCLC-I |
| chr12: 57737630 | ENST00000257897.7 | AGAP2 | Intron | SCLC-I |
| chr12: 57738134 | ENST00000257897.7 | AGAP2 | Intron | SCLC-I |
| chr12: 57738397 | ENST00000257897.7 | AGAP2 | Intron | SCLC-I |
| chr12: 80708706 | ENST00000228641.4 | MYF6 | Intron | SCLC-I |
| chr12: 80708732 | ENST00000228641.4 | MYF6 | Intron | SCLC-I |
| chr12: 124463522 | ENST00000448008.1 | NCOR2 | Intron | SCLC-I |
| chr12: 128939360 | ENST00000281703.11 | GLT1D1 | Intron | SCLC-I |
| chr13: 25973246 | ENST00000381655.7 | ATP8A2 | Intron | SCLC-I |
| chr13: 48319241 | ENST00000467505.5 | RB1 | Intron | SCLC-I |
| chr13: 48319243 | ENST00000467505.5 | RB1 | Intron | SCLC-I |
| chr13: 48319263 | ENST00000467505.5 | RB1 | Intron | SCLC-I |
| chr13: 48319281 | ENST00000467505.5 | RB1 | Intron | SCLC-I |
| chr13: 48319291 | ENST00000467505.5 | RB1 | Intron | SCLC-I |
| chr13: 48319306 | ENST00000467505.5 | RB1 | Intron | SCLC-I |
| chr13: 50132310 | ENST00000476738.5 | DLEU1 | Intron | SCLC-I |
| chr13: 50133486 | ENST00000476738.5 | DLEU1 | Intron | SCLC-I |
| chr13: 50133529 | ENST00000476738.5 | DLEU1 | Intron | SCLC-I |
| chr13: 50133537 | ENST00000476738.5 | DLEU1 | Intron | SCLC-I |
| chr13: 50133563 | ENST00000476738.5 | DLEU1 | Intron | SCLC-I |
| chr13: 50133576 | ENST00000476738.5 | DLEU1 | Intron | SCLC-I |
| chr13: 50133608 | ENST00000476738.5 | DLEU1 | Intron | SCLC-I |
| chr13: 78607453 | ENST00000560209.6 | OBI1-AS1 | Intron | SCLC-I |
| chr13: 78607812 | ENST00000560209.6 | OBI1-AS1 | Intron | SCLC-I |
| chr14: 36579935 | ENST00000634305.1 | NA | Intron | SCLC-I |
| chr14: 37602183 | ENST00000556845.1 | TTC6 | Intron | SCLC-I |
| chr14: 38256100 | ENST00000655453.1 | NA | Promoter | SCLC-I |
| chr14: 38256107 | ENST00000655453.1 | NA | Promoter | SCLC-I |
| chr14: 60515473 | ENST00000557618.1 | NA | Intron | SCLC-I |
| chr14: 60515555 | ENST00000557618.1 | NA | Intron | SCLC-I |
| chr14: 60515579 | ENST00000557618.1 | NA | Intron | SCLC-I |
| chr14: 60515651 | ENST00000557618.1 | NA | Intron | SCLC-I |
| chr14: 60515654 | ENST00000557618.1 | NA | Intron | SCLC-I |
| chr14: 60515656 | ENST00000557618.1 | NA | Intron | SCLC-I |
| chr14: 60515669 | ENST00000557618.1 | NA | Intron | SCLC-I |
| chr14: 60515671 | ENST00000557618.1 | NA | Intron | SCLC-I |
| chr14: 60515691 | ENST00000557618.1 | NA | Intron | SCLC-I |
| chr14: 60515705 | ENST00000557618.1 | NA | Intron | SCLC-I |
| chr14: 65287668 | ENST00000555736.1 | NA | Intron | SCLC-I |
| chr14: 65287696 | ENST00000555736.1 | NA | Intron | SCLC-I |
| chr14: 65287718 | ENST00000555736.1 | NA | Intron | SCLC-I |
| chr14: 65287767 | ENST00000555736.1 | NA | Intron | SCLC-I |
| chr14: 65287768 | ENST00000555736.1 | NA | Intron | SCLC-I |
| chr14: 69944274 | ENST00000555917.1 | SMOC1 | Intron | SCLC-I |
| chr14: 74922831 | ENST00000555834.5 | RPS6KL1 | Intron | SCLC-I |
| chr14: 74922839 | ENST00000555834.5 | RPS6KL1 | Intron | SCLC-I |
| chr14: 74922865 | ENST00000555834.5 | RPS6KL1 | Intron | SCLC-I |
| chr14: 74922893 | ENST00000555834.5 | RPS6KL1 | Intron | SCLC-I |
| chr14: 74922915 | ENST00000555834.5 | RPS6KL1 | Intron | SCLC-I |
| chr14: 74922930 | ENST00000555834.5 | RPS6KL1 | Intron | SCLC-I |
| chr14: 77025612 | ENST00000238647.5 | IRF2BPL | Exon | SCLC-I |
| chr14: 77025619 | ENST00000238647.5 | IRF2BPL | Exon | SCLC-I |
| chr14: 77025636 | ENST00000238647.5 | IRF2BPL | Exon | SCLC-I |
| chr14: 77025685 | ENST00000238647.5 | IRF2BPL | Exon | SCLC-I |
| chr14: 77025829 | ENST00000238647.5 | IRF2BPL | Exon | SCLC-I |
| chr14: 77025834 | ENST00000238647.5 | IRF2BPL | Exon | SCLC-I |
| chr14: 77025840 | ENST00000238647.5 | IRF2BPL | Exon | SCLC-I |
| chr14: 77025857 | ENST00000238647.5 | IRF2BPL | Exon | SCLC-I |
| chr14: 77026043 | ENST00000238647.5 | IRF2BPL | Exon | SCLC-I |
| chr14: 77026046 | ENST00000238647.5 | IRF2BPL | Exon | SCLC-I |
| chr14: 77026047 | ENST00000238647.5 | IRF2BPL | Exon | SCLC-I |
| chr14: 77026070 | ENST00000238647.5 | IRF2BPL | Exon | SCLC-I |
| chr14: 77026080 | ENST00000238647.5 | IRF2BPL | Exon | SCLC-I |
| chr14: 77026109 | ENST00000238647.5 | IRF2BPL | Exon | SCLC-I |
| chr14: 77026122 | ENST00000238647.5 | IRF2BPL | Exon | SCLC-I |
| chr14: 77026124 | ENST00000238647.5 | IRF2BPL | Exon | SCLC-I |
| chr14: 77026130 | ENST00000238647.5 | IRF2BPL | Exon | SCLC-I |
| chr14: 77026236 | ENST00000238647.5 | IRF2BPL | Exon | SCLC-I |
| chr14: 77026245 | ENST00000238647.5 | IRF2BPL | Exon | SCLC-I |
| chr14: 77026247 | ENST00000238647.5 | IRF2BPL | Exon | SCLC-I |
| chr14: 77026290 | ENST00000238647.5 | IRF2BPL | Exon | SCLC-I |
| chr14: 77026317 | ENST00000238647.5 | IRF2BPL | Exon | SCLC-I |
| chr14: 77026323 | ENST00000238647.5 | IRF2BPL | Exon | SCLC-I |
| chr14: 77026441 | ENST00000238647.5 | IRF2BPL | Exon | SCLC-I |
| chr14: 77026445 | ENST00000238647.5 | IRF2BPL | Exon | SCLC-I |
| chr14: 77026559 | ENST00000238647.5 | IRF2BPL | Exon | SCLC-I |
| chr14: 77026571 | ENST00000238647.5 | IRF2BPL | Exon | SCLC-I |
| chr14: 77026650 | ENST00000238647.5 | IRF2BPL | Exon | SCLC-I |
| chr14: 77026837 | ENST00000238647.5 | IRF2BPL | Exon | SCLC-I |
| chr14: 77026840 | ENST00000238647.5 | IRF2BPL | Exon | SCLC-I |
| chr14: 91375740 | ENST00000389857.11 | CCDC88C | Intron | SCLC-I |
| chr14: 92687670 | ENST00000556418.1 | RIN3 | Exon | SCLC-I |
| chr14: 92687692 | ENST00000556418.1 | RIN3 | Exon | SCLC-I |
| chr14: 92687699 | ENST00000556418.1 | RIN3 | Exon | SCLC-I |
| chr14: 92687725 | ENST00000556418.1 | RIN3 | Exon | SCLC-I |
| chr14: 92687758 | ENST00000556418.1 | RIN3 | Exon | SCLC-I |
| chr14: 92687767 | ENST00000556418.1 | RIN3 | Exon | SCLC-I |
| chr14: 92687795 | ENST00000556418.1 | RIN3 | Exon | SCLC-I |
| chr14: 95191817 | ENST00000556416.1 | CLMN | Exon | SCLC-I |
| chr14: 95714410 | ENST00000547644.6 | LOC107984703 | Intron | SCLC-I |
| chr14: 104103193 | ENST00000551170.1 | ASPG | Intron | SCLC-I |
| chr14: 105060914 | ENST00000546679.1 | GPR132 | Intron | SCLC-I |
| chr15: 34018462 | ENST00000560035.1 | CHRM5 | Intron | SCLC-I |
| chr15: 42608310 | ENST00000563872.5 | STARD9 | Intron | SCLC-I |
| chr15: 45650342 | ENST00000564080.1 | SQOR | Intron | SCLC-I |
| chr15: 45650351 | ENST00000564080.1 | SQOR | Intron | SCLC-I |
| chr15: 47985845 | ENST00000664705.1 | NA | Intron | SCLC-I |
| chr15: 47985925 | ENST00000664705.1 | NA | Intron | SCLC-I |
| chr15: 65028370 | ENST00000220058.9 | MTFMT | Intron | SCLC-I |
| chr15: 65028409 | ENST00000220058.9 | MTFMT | Intron | SCLC-I |
| chr15: 92623668 | ENST00000553393.5 | FAM174B | Intron | SCLC-I |
| chr15: 93041663 | ENST00000329082.11 | RGMA | Exon | SCLC-I |
| chr16: 3027967 | ENST00000572240.5 | BICDL2 | Exon | SCLC-I |
| chr16: 20349066 | ENST00000396134.6 | UMOD | Exon | SCLC-I |
| chr16: 20349093 | ENST00000396134.6 | UMOD | Exon | SCLC-I |
| chr16: 30751128 | ENST00000569762.1 | PHKG2 | Exon | SCLC-I |
| chr16: 54288396 | ENST00000637770.1 | NA | Intron | SCLC-I |
| chr16: 54288536 | ENST00000637770.1 | NA | Intron | SCLC-I |
| chr16: 54288547 | ENST00000637770.1 | NA | Intron | SCLC-I |
| chr16: 57894225 | ENST00000565942.1 | CNGB1 | Intron | SCLC-I |
| chr16: 67165147 | ENST00000518753.5 | HSF4 | Intron | SCLC-I |
| chr16: 67165148 | ENST00000518753.5 | HSF4 | Intron | SCLC-I |
| chr16: 67165753 | ENST00000519224.5 | HSF4 | Promoter | SCLC-I |
| chr16: 67284771 | ENST00000393966.1 | PLEKHG4 | Intron | SCLC-I |
| chr16: 67441430 | ENST00000568620.5 | ATP6V0D1 | Exon | SCLC-I |
| chr16: 67653815 | ENST00000334583.11 | CARMIL2 | Intron | SCLC-I |
| chr16: 67653842 | ENST00000334583.11 | CARMIL2 | Intron | SCLC-I |
| chr16: 67653852 | ENST00000334583.11 | CARMIL2 | Intron | SCLC-I |
| chr16: 68235387 | ENST00000473183.7 | ESRP2 | Intron | SCLC-I |
| chr16: 68235392 | ENST00000473183.7 | ESRP2 | Intron | SCLC-I |
| chr16: 68235401 | ENST00000473183.7 | ESRP2 | Intron | SCLC-I |
| chr16: 68235462 | ENST00000473183.7 | ESRP2 | Intron | SCLC-I |
| chr16: 68235464 | ENST00000473183.7 | ESRP2 | Intron | SCLC-I |
| chr16: 68235479 | ENST00000473183.7 | ESRP2 | Intron | SCLC-I |
| chr16: 68235485 | ENST00000473183.7 | ESRP2 | Intron | SCLC-I |
| chr16: 68235487 | ENST00000473183.7 | ESRP2 | Intron | SCLC-I |
| chr16: 68235494 | ENST00000473183.7 | ESRP2 | Intron | SCLC-I |
| chr16: 68235527 | ENST00000473183.7 | ESRP2 | Intron | SCLC-I |
| chr16: 68235695 | ENST00000562567.6 | ESRP2 | Promoter | SCLC-I |
| chr16: 85220415 | ENST00000637419.1 | GSE1 | Intron | SCLC-I |
| chr16: 85282587 | ENST00000637419.1 | GSE1 | Intron | SCLC-I |
| chr16: 85382138 | ENST00000637419.1 | GSE1 | Intron | SCLC-I |
| chr16: 85449451 | ENST00000637419.1 | GSE1 | Intron | SCLC-I |
| chr16: 85449457 | ENST00000637419.1 | GSE1 | Intron | SCLC-I |
| chr16: 85452157 | ENST00000637419.1 | GSE1 | Intron | SCLC-I |
| chr16: 85452179 | ENST00000637419.1 | GSE1 | Intron | SCLC-I |
| chr16: 85452187 | ENST00000637419.1 | GSE1 | Intron | SCLC-I |
| chr16: 85452198 | ENST00000637419.1 | GSE1 | Intron | SCLC-I |
| chr16: 85452271 | ENST00000637419.1 | GSE1 | Intron | SCLC-I |
| chr16: 85452273 | ENST00000637419.1 | GSE1 | Intron | SCLC-I |
| chr16: 85485649 | ENST00000637419.1 | GSE1 | Intron | SCLC-I |
| chr16: 85594161 | ENST00000637419.1 | GSE1 | Intron | SCLC-I |
| chr16: 88505436 | ENST00000563351.5 | ZFPM1 | Intron | SCLC-I |
| chr16: 88505689 | ENST00000563351.5 | ZFPM1 | Intron | SCLC-I |
| chr16: 88505707 | ENST00000563351.5 | ZFPM1 | Intron | SCLC-I |
| chr16: 88703333 | ENST00000567844.1 | RNF166 | Intron | SCLC-I |
| chr16: 88703359 | ENST00000567844.1 | RNF166 | Intron | SCLC-I |
| chr17: 4600012 | ENST00000338859.8 | SMTNL2 | Intron | SCLC-I |
| chr17: 4600024 | ENST00000338859.8 | SMTNL2 | Intron | SCLC-I |
| chr17: 4600031 | ENST00000338859.8 | SMTNL2 | Intron | SCLC-I |
| chr17: 4600044 | ENST00000338859.8 | SMTNL2 | Intron | SCLC-I |
| chr17: 8799394 | ENST00000329805.6 | MFSD6L | Promoter | SCLC-I |
| chr17: 8799405 | ENST00000329805.6 | MFSD6L | Promoter | SCLC-I |
| chr17: 8799413 | ENST00000329805.6 | MFSD6L | Promoter | SCLC-I |
| chr17: 8799419 | ENST00000329805.6 | MFSD6L | Promoter | SCLC-I |
| chr17: 8799421 | ENST00000329805.6 | MFSD6L | Promoter | SCLC-I |
| chr17: 8799434 | ENST00000329805.6 | MFSD6L | Promoter | SCLC-I |
| chr17: 34637675 | ENST00000321639.7 | TMEM132E | Exon | SCLC-I |
| chr17: 34637684 | ENST00000321639.7 | TMEM132E | Exon | SCLC-I |
| chr17: 34637686 | ENST00000321639.7 | TMEM132E | Exon | SCLC-I |
| chr17: 38561771 | ENST00000617146.5 | SRCIN1 | Exon | SCLC-I |
| chr17: 38561774 | ENST00000617146.5 | SRCIN1 | Exon | SCLC-I |
| chr17: 38561777 | ENST00000617146.5 | SRCIN1 | Exon | SCLC-I |
| chr17: 38561784 | ENST00000617146.5 | SRCIN1 | Exon | SCLC-I |
| chr17: 38561847 | ENST00000617146.5 | SRCIN1 | Exon | SCLC-I |
| chr17: 38561862 | ENST00000617146.5 | SRCIN1 | Exon | SCLC-I |
| chr17: 45241394 | ENST00000587856.1 | FMNL1 | Exon | SCLC-I |
| chr17: 45241459 | ENST00000587856.1 | FMNL1 | Exon | SCLC-I |
| chr17: 45241470 | ENST00000587856.1 | FMNL1 | Exon | SCLC-I |
| chr17: 45241527 | ENST00000587856.1 | FMNL1 | Exon | SCLC-I |
| chr17: 45247010 | ENST00000420431.2 | NA | Intron | SCLC-I |
| chr17: 48838028 | ENST00000508679.5 | CALCOCO2 | Intron | SCLC-I |
| chr17: 48838036 | ENST00000508679.5 | CALCOCO2 | Intron | SCLC-I |
| chr17: 58487554 | ENST00000323777.8 | HSF5 | Intron | SCLC-I |
| chr17: 58487572 | ENST00000323777.8 | HSF5 | Intron | SCLC-I |
| chr17: 58487581 | ENST00000323777.8 | HSF5 | Intron | SCLC-I |
| chr17: 58488117 | ENST00000323777.8 | HSF5 | Exon | SCLC-I |
| chr17: 58488136 | ENST00000323777.8 | HSF5 | Exon | SCLC-I |
| chr17: 58488137 | ENST00000323777.8 | HSF5 | Exon | SCLC-I |
| chr17: 58488151 | ENST00000323777.8 | HSF5 | Exon | SCLC-I |
| chr17: 58488172 | ENST00000323777.8 | HSF5 | Exon | SCLC-I |
| chr17: 58488175 | ENST00000323777.8 | HSF5 | Exon | SCLC-I |
| chr17: 58488183 | ENST00000323777.8 | HSF5 | Exon | SCLC-I |
| chr17: 58488198 | ENST00000323777.8 | HSF5 | Exon | SCLC-I |
| chr17: 58488200 | ENST00000323777.8 | HSF5 | Exon | SCLC-I |
| chr17: 58488201 | ENST00000323777.8 | HSF5 | Exon | SCLC-I |
| chr17: 58488216 | ENST00000323777.8 | HSF5 | Exon | SCLC-I |
| chr17: 58488280 | ENST00000323777.8 | HSF5 | Exon | SCLC-I |
| chr17: 58488281 | ENST00000323777.8 | HSF5 | Exon | SCLC-I |
| chr17: 63420277 | ENST00000583552.1 | NA | Intron | SCLC-I |
| chr17: 63420295 | ENST00000583552.1 | NA | Intron | SCLC-I |
| chr17: 63420387 | ENST00000583552.1 | NA | Intron | SCLC-I |
| chr17: 63420388 | ENST00000583552.1 | NA | Intron | SCLC-I |
| chr17: 72116717 | ENST00000529667.1 | SOX9-AS1 | Intron | SCLC-I |
| chr17: 72116724 | ENST00000529667.1 | SOX9-AS1 | Intron | SCLC-I |
| chr17: 72589138 | ENST00000648088.1 | LINC00673 | Intron | SCLC-I |
| chr17: 72589198 | ENST00000648088.1 | LINC00673 | Intron | SCLC-I |
| chr17: 75012406 | ENST00000584208.5 | MRPL58 | Promoter | SCLC-I |
| chr17: 75141194 | ENST00000356033.8 | JPT1 | Intron | SCLC-I |
| chr17: 75141237 | ENST00000356033.8 | JPT1 | Intron | SCLC-I |
| chr17: 77453652 | ENST00000588575.1 | SEPTIN9 | Intron | SCLC-I |
| chr17: 77453692 | ENST00000588575.1 | SEPTIN9 | Intron | SCLC-I |
| chr17: 81135920 | ENST00000326724.9 | AATK | Intron | SCLC-I |
| chr17: 81136020 | ENST00000326724.9 | AATK | Intron | SCLC-I |
| chr17: 81136037 | ENST00000326724.9 | AATK | Intron | SCLC-I |
| chr17: 81136041 | ENST00000326724.9 | AATK | Intron | SCLC-I |
| chr17: 81136066 | ENST00000326724.9 | AATK | Intron | SCLC-I |
| chr17: 81136085 | ENST00000326724.9 | AATK | Intron | SCLC-I |
| chr17: 81136104 | ENST00000326724.9 | AATK | Intron | SCLC-I |
| chr17: 81136111 | ENST00000326724.9 | AATK | Intron | SCLC-I |
| chr19: 1071166 | ENST00000591293.2 | ARHGAP45 | Intron | SCLC-I |
| chr19: 1071186 | ENST00000591293.2 | ARHGAP45 | Intron | SCLC-I |
| chr19: 1083008 | ENST00000586378.5 | ARHGAP45 | Exon | SCLC-I |
| chr19: 2274694 | ENST00000621615.1 | SPPL2B | Intron | SCLC-I |
| chr19: 2274698 | ENST00000621615.1 | SPPL2B | Intron | SCLC-I |
| chr19: 2274700 | ENST00000621615.1 | SPPL2B | Intron | SCLC-I |
| chr19: 2274710 | ENST00000621615.1 | SPPL2B | Intron | SCLC-I |
| chr19: 2274754 | ENST00000621615.1 | SPPL2B | Intron | SCLC-I |
| chr19: 3179282 | ENST00000246115.5 | S1PR4 | Exon | SCLC-I |
| chr19: 3179291 | ENST00000246115.5 | S1PR4 | Exon | SCLC-I |
| chr19: 3179367 | ENST00000246115.5 | S1PR4 | Exon | SCLC-I |
| chr19: 5046476 | ENST00000611640.4 | KDM4B | Intron | SCLC-I |
| chr19: 5046532 | ENST00000611640.4 | KDM4B | Intron | SCLC-I |
| chr19: 5046687 | ENST00000611640.4 | KDM4B | Intron | SCLC-I |
| chr19: 5046728 | ENST00000611640.4 | KDM4B | Intron | SCLC-I |
| chr19: 8526066 | ENST00000644032.2 | MYO1F | Intron | SCLC-I |
| chr19: 8526099 | ENST00000644032.2 | MYO1F | Intron | SCLC-I |
| chr19: 9883533 | ENST00000264833.8 | OLFM2 | Intron | SCLC-I |
| chr19: 9883534 | ENST00000264833.8 | OLFM2 | Intron | SCLC-I |
| chr19: 9883653 | ENST00000264833.8 | OLFM2 | Intron | SCLC-I |
| chr19: 9883659 | ENST00000264833.8 | OLFM2 | Intron | SCLC-I |
| chr19: 9883675 | ENST00000264833.8 | OLFM2 | Intron | SCLC-I |
| chr19: 10334198 | ENST00000612689.1 | NA | Exon | SCLC-I |
| chr19: 10334209 | ENST00000612689.1 | NA | Exon | SCLC-I |
| chr19: 10334241 | ENST00000612689.1 | NA | Exon | SCLC-I |
| chr19: 10334247 | ENST00000612689.1 | NA | Exon | SCLC-I |
| chr19: 10334670 | ENST00000612689.1 | NA | Exon | SCLC-I |
| chr19: 10334678 | ENST00000612689.1 | NA | Exon | SCLC-I |
| chr19: 10334698 | ENST00000612689.1 | NA | Exon | SCLC-I |
| chr19: 10334720 | ENST00000612689.1 | NA | Exon | SCLC-I |
| chr19: 10334744 | ENST00000612689.1 | NA | Exon | SCLC-I |
| chr19: 10625535 | ENST00000590382.5 | SLC44A2 | Intron | SCLC-I |
| chr19: 10625547 | ENST00000590382.5 | SLC44A2 | Intron | SCLC-I |
| chr19: 10625549 | ENST00000590382.5 | SLC44A2 | Intron | SCLC-I |
| chr19: 10625581 | ENST00000590382.5 | SLC44A2 | Intron | SCLC-I |
| chr19: 10625589 | ENST00000590382.5 | SLC44A2 | Intron | SCLC-I |
| chr19: 10625607 | ENST00000590382.5 | SLC44A2 | Intron | SCLC-I |
| chr19: 10625611 | ENST00000590382.5 | SLC44A2 | Intron | SCLC-I |
| chr19: 10625613 | ENST00000590382.5 | SLC44A2 | Intron | SCLC-I |
| chr19: 10625614 | ENST00000590382.5 | SLC44A2 | Intron | SCLC-I |
| chr19: 10625623 | ENST00000590382.5 | SLC44A2 | Intron | SCLC-I |
| chr19: 10625624 | ENST00000590382.5 | SLC44A2 | Intron | SCLC-I |
| chr19: 10625642 | ENST00000590382.5 | SLC44A2 | Intron | SCLC-I |
| chr19: 10625646 | ENST00000590382.5 | SLC44A2 | Intron | SCLC-I |
| chr19: 10625647 | ENST00000590382.5 | SLC44A2 | Intron | SCLC-I |
| chr19: 10625669 | ENST00000590382.5 | SLC44A2 | Intron | SCLC-I |
| chr19: 10625670 | ENST00000590382.5 | SLC44A2 | Intron | SCLC-I |
| chr19: 10625679 | ENST00000590382.5 | SLC44A2 | Intron | SCLC-I |
| chr19: 10625680 | ENST00000590382.5 | SLC44A2 | Intron | SCLC-I |
| chr19: 10625686 | ENST00000590382.5 | SLC44A2 | Intron | SCLC-I |
| chr19: 10625687 | ENST00000590382.5 | SLC44A2 | Intron | SCLC-I |
| chr19: 10625690 | ENST00000590382.5 | SLC44A2 | Intron | SCLC-I |
| chr19: 10625691 | ENST00000590382.5 | SLC44A2 | Intron | SCLC-I |
| chr19: 10625699 | ENST00000590382.5 | SLC44A2 | Intron | SCLC-I |
| chr19: 10625848 | ENST00000590382.5 | SLC44A2 | Intron | SCLC-I |
| chr19: 10625854 | ENST00000590382.5 | SLC44A2 | Intron | SCLC-I |
| chr19: 14440401 | ENST00000585619.5 | PKN1 | Intron | SCLC-I |
| chr19: 15457540 | ENST00000599694.1 | RASAL3 | Promoter | SCLC-I |
| chr19: 15457550 | ENST00000599694.1 | RASAL3 | Promoter | SCLC-I |
| chr19: 15457577 | ENST00000599694.1 | RASAL3 | Promoter | SCLC-I |
| chr19: 15457579 | ENST00000599694.1 | RASAL3 | Promoter | SCLC-I |
| chr19: 15457599 | ENST00000599694.1 | RASAL3 | Promoter | SCLC-I |
| chr19: 17357872 | ENST00000595816.1 | PLVAP | Intron | SCLC-I |
| chr19: 17357883 | ENST00000595816.1 | PLVAP | Intron | SCLC-I |
| chr19: 18762413 | ENST00000338797.10 | CRTC1 | Intron | SCLC-I |
| chr19: 32971400 | ENST00000305768.10 | CEP89 | Intron | SCLC-I |
| chr19: 35033965 | ENST00000415950.4 | SCN1B | Exon | SCLC-I |
| chr19: 35249607 | ENST00000597933.5 | LSR | Intron | SCLC-I |
| chr19: 35249625 | ENST00000597933.5 | LSR | Intron | SCLC-I |
| chr19: 35249634 | ENST00000597933.5 | LSR | Intron | SCLC-I |
| chr19: 35249672 | ENST00000597933.5 | LSR | Intron | SCLC-I |
| chr19: 38596247 | ENST00000589557.1 | LOC105372397 | Promoter | SCLC-I |
| chr19: 46494426 | ENST00000602017.5 | PPP5D1 | Intron | SCLC-I |
| chr19: 46494440 | ENST00000602017.5 | PPP5D1 | Intron | SCLC-I |
| chr19: 46494443 | ENST00000602017.5 | PPP5D1 | Intron | SCLC-I |
| chr19: 46494448 | ENST00000602017.5 | PPP5D1 | Intron | SCLC-I |
| chr19: 46494469 | ENST00000602017.5 | PPP5D1 | Intron | SCLC-I |
| chr19: 48552206 | ENST00000666424.1 | NA | Intron | SCLC-I |
| chr19: 48552207 | ENST00000666424.1 | NA | Intron | SCLC-I |
| chr19: 49690337 | ENST00000594038.5 | CPT1C | Promoter | SCLC-I |
| chr19: 49690351 | ENST00000594038.5 | CPT1C | Promoter | SCLC-I |
| chr19: 49690370 | ENST00000594038.5 | CPT1C | Promoter | SCLC-I |
| chr19: 49690375 | ENST00000594038.5 | CPT1C | Promoter | SCLC-I |
| chr2: 8545830 | ENST00000670008.1 | LINC01814 | Intron | SCLC-I |
| chr2: 8545868 | ENST00000670008.1 | LINC01814 | Intron | SCLC-I |
| chr2: 19356109 | ENST00000487581.1 | OSR1 | Exon | SCLC-I |
| chr2: 19356152 | ENST00000487581.1 | OSR1 | Exon | SCLC-I |
| chr2: 19356186 | ENST00000487581.1 | OSR1 | Exon | SCLC-I |
| chr2: 19356305 | ENST00000487581.1 | OSR1 | Exon | SCLC-I |
| chr2: 27308111 | ENST00000296099.2 | UCN | Intron | SCLC-I |
| chr2: 27308460 | ENST00000296099.2 | UCN | Promoter | SCLC-I |
| chr2: 27308503 | ENST00000296099.2 | UCN | Promoter | SCLC-I |
| chr2: 27442403 | ENST00000407293.5 | KRTCAP3 | Promoter | SCLC-I |
| chr2: 27442440 | ENST00000407293.5 | KRTCAP3 | Promoter | SCLC-I |
| chr2: 27442450 | ENST00000464699.5 | KRTCAP3 | Promoter | SCLC-I |
| chr2: 27442568 | ENST00000464699.5 | KRTCAP3 | Promoter | SCLC-I |
| chr2: 27442588 | ENST00000464699.5 | KRTCAP3 | Promoter | SCLC-I |
| chr2: 60484506 | ENST00000646249.1 | BCL11A | Intron | SCLC-I |
| chr2: 63051888 | ENST00000366671.7 | OTX1 | Intron | SCLC-I |
| chr2: 63052917 | ENST00000484066.2 | OTX1 | Exon | SCLC-I |
| chr2: 63055800 | ENST00000366671.7 | OTX1 | Exon | SCLC-I |
| chr2: 65430462 | ENST00000356388.9 | SPRED2 | Intron | SCLC-I |
| chr2: 66582648 | ENST00000433396.5 | LINC01798 | Intron | SCLC-I |
| chr2: 68365462 | ENST00000234313.8 | PLEK | Intron | SCLC-I |
| chr2: 70904761 | ENST00000234392.3 | VAX2 | Intron | SCLC-I |
| chr2: 70907108 | ENST00000234392.3 | VAX2 | Intron | SCLC-I |
| chr2: 73269181 | ENST00000521871.5 | FBXO41 | Exon | SCLC-I |
| chr2: 97713599 | ENST00000264972.9 | ZAP70 | UTR5 | SCLC-I |
| chr2: 100103855 | ENST00000409236.6 | AFF3 | Intron | SCLC-I |
| chr2: 100103888 | ENST00000409236.6 | AFF3 | Intron | SCLC-I |
| chr2: 100103931 | ENST00000409236.6 | AFF3 | Intron | SCLC-I |
| chr2: 102505500 | ENST00000492154.1 | SLC9A4 | Exon | SCLC-I |
| chr2: 102505583 | ENST00000492154.1 | SLC9A4 | Exon | SCLC-I |
| chr2: 109614831 | ENST00000415095.5 | SEPTIN10 | Promoter | SCLC-I |
| chr2: 109614835 | ENST00000415095.5 | SEPTIN10 | Promoter | SCLC-I |
| chr2: 109614850 | ENST00000437928.5 | SEPTIN10 | Promoter | SCLC-I |
| chr2: 109614852 | ENST00000437928.5 | SEPTIN10 | Promoter | SCLC-I |
| chr2: 113199049 | ENST00000409378.1 | PSD4 | Intron | SCLC-I |
| chr2: 113199067 | ENST00000409378.1 | PSD4 | Intron | SCLC-I |
| chr2: 156329800 | ENST00000406048.2 | NR4A2 | Intron | SCLC-I |
| chr2: 156329803 | ENST00000406048.2 | NR4A2 | Intron | SCLC-I |
| chr2: 156329809 | ENST00000406048.2 | NR4A2 | Intron | SCLC-I |
| chr20: 33568553 | ENST00000471007.1 | CBFA2T2 | Intron | SCLC-I |
| chr20: 33568556 | ENST00000471007.1 | CBFA2T2 | Intron | SCLC-I |
| chr20: 33568560 | ENST00000471007.1 | CBFA2T2 | Intron | SCLC-I |
| chr20: 33568562 | ENST00000471007.1 | CBFA2T2 | Intron | SCLC-I |
| chr20: 37519209 | ENST00000467603.1 | BLCAP | Intron | SCLC-I |
| chr20: 37519228 | ENST00000467603.1 | BLCAP | Intron | SCLC-I |
| chr20: 37519303 | ENST00000397137.5 | BLCAP | Exon | SCLC-I |
| chr20: 37519327 | ENST00000397137.5 | BLCAP | Exon | SCLC-I |
| chr20: 49916348 | ENST00000289431.10 | SPATA2 | Promoter | SCLC-I |
| chr20: 49916358 | ENST00000289431.10 | SPATA2 | Promoter | SCLC-I |
| chr20: 49916379 | ENST00000289431.10 | SPATA2 | Promoter | SCLC-I |
| chr20: 50624748 | ENST00000327979.8 | RIPOR3 | Intron | SCLC-I |
| chr20: 62433875 | ENST00000618678.2 | NA | Intron | SCLC-I |
| chr20: 64091986 | ENST00000355631.8 | OPRL1 | Intron | SCLC-I |
| chr20: 64091995 | ENST00000355631.8 | OPRL1 | Intron | SCLC-I |
| chr21: 42761195 | ENST00000291539.11 | PDE9A | Intron | SCLC-I |
| chr21: 42781489 | ENST00000420273.2 | NA | Exon | SCLC-I |
| chr21: 42781516 | ENST00000420273.2 | NA | Exon | SCLC-I |
| chr21: 42781722 | ENST00000420273.2 | NA | Exon | SCLC-I |
| chr21: 44370400 | ENST00000300482.9 | TRPM2 | Intron | SCLC-I |
| chr21: 44375502 | ENST00000300482.9 | TRPM2 | Intron | SCLC-I |
| chr21: 44704883 | ENST00000323084.9 | TSPEAR | Intron | SCLC-I |
| chr21: 44706160 | ENST00000323084.9 | TSPEAR | Intron | SCLC-I |
| chr21: 44706163 | ENST00000323084.9 | TSPEAR | Intron | SCLC-I |
| chr21: 44706566 | ENST00000323084.9 | TSPEAR | Intron | SCLC-I |
| chr21: 44706573 | ENST00000323084.9 | TSPEAR | Intron | SCLC-I |
| chr21: 44706588 | ENST00000323084.9 | TSPEAR | Intron | SCLC-I |
| chr21: 44706603 | ENST00000323084.9 | TSPEAR | Intron | SCLC-I |
| chr22: 25779932 | ENST00000536101.5 | MYO18B | Intron | SCLC-I |
| chr22: 25779973 | ENST00000536101.5 | MYO18B | Intron | SCLC-I |
| chr22: 26645209 | ENST00000425548.1 | NA | Exon | SCLC-I |
| chr22: 38291585 | ENST00000396832.6 | CSNK1E | Exon | SCLC-I |
| chr22: 38291626 | ENST00000396832.6 | CSNK1E | Exon | SCLC-I |
| chr22: 38291631 | ENST00000396832.6 | CSNK1E | Exon | SCLC-I |
| chr22: 42314379 | ENST00000515426.1 | TCF20 | Intron | SCLC-I |
| chr22: 42314380 | ENST00000515426.1 | TCF20 | Intron | SCLC-I |
| chr22: 43042239 | ENST00000266254.12 | TTLL1 | Intron | SCLC-I |
| chr22: 43042247 | ENST00000266254.12 | TTLL1 | Intron | SCLC-I |
| chr22: 49808156 | ENST00000438393.5 | BRD1 | Intron | SCLC-I |
| chr22: 49808180 | ENST00000438393.5 | BRD1 | Intron | SCLC-I |
| chr22: 49925568 | ENST00000483652.5 | CRELD2 | Exon | SCLC-I |
| chr22: 50603738 | ENST00000329492.6 | MAPK8IP2 | Intron | SCLC-I |
| chr22: 50603740 | ENST00000329492.6 | MAPK8IP2 | Intron | SCLC-I |
| chr22: 50603745 | ENST00000329492.6 | MAPK8IP2 | Intron | SCLC-I |
| chr22: 50603747 | ENST00000329492.6 | MAPK8IP2 | Intron | SCLC-I |
| chr22: 50603755 | ENST00000329492.6 | MAPK8IP2 | Intron | SCLC-I |
| chr22: 50604086 | ENST00000329492.6 | MAPK8IP2 | Exon | SCLC-I |
| chr22: 50604120 | ENST00000329492.6 | MAPK8IP2 | Exon | SCLC-I |
| chr22: 50604139 | ENST00000329492.6 | MAPK8IP2 | Exon | SCLC-I |
| chr22: 50604146 | ENST00000329492.6 | MAPK8IP2 | Exon | SCLC-I |
| chr22: 50604201 | ENST00000329492.6 | MAPK8IP2 | Exon | SCLC-I |
| chr22: 50604208 | ENST00000329492.6 | MAPK8IP2 | Exon | SCLC-I |
| chr22: 50604219 | ENST00000329492.6 | MAPK8IP2 | Exon | SCLC-I |
| chr22: 50604229 | ENST00000329492.6 | MAPK8IP2 | Exon | SCLC-I |
| chr22: 50604231 | ENST00000329492.6 | MAPK8IP2 | Exon | SCLC-I |
| chr22: 50604233 | ENST00000329492.6 | MAPK8IP2 | Exon | SCLC-I |
| chr22: 50604241 | ENST00000329492.6 | MAPK8IP2 | Exon | SCLC-I |
| chr22: 50604242 | ENST00000329492.6 | MAPK8IP2 | Exon | SCLC-I |
| chr22: 50604243 | ENST00000329492.6 | MAPK8IP2 | Exon | SCLC-I |
| chr22: 50604246 | ENST00000329492.6 | MAPK8IP2 | Exon | SCLC-I |
| chr22: 50604251 | ENST00000329492.6 | MAPK8IP2 | Exon | SCLC-I |
| chr22: 50604272 | ENST00000329492.6 | MAPK8IP2 | Exon | SCLC-I |
| chr22: 50604293 | ENST00000329492.6 | MAPK8IP2 | Exon | SCLC-I |
| chr22: 50604297 | ENST00000329492.6 | MAPK8IP2 | Exon | SCLC-I |
| chr22: 50604298 | ENST00000329492.6 | MAPK8IP2 | Exon | SCLC-I |
| chr22: 50604304 | ENST00000329492.6 | MAPK8IP2 | Exon | SCLC-I |
| chr22: 50604310 | ENST00000329492.6 | MAPK8IP2 | Exon | SCLC-I |
| chr22: 50604316 | ENST00000329492.6 | MAPK8IP2 | Exon | SCLC-I |
| chr22: 50604514 | ENST00000329492.6 | MAPK8IP2 | Exon | SCLC-I |
| chr22: 50697638 | ENST00000414786.6 | SHANK3 | Exon | SCLC-I |
| chr22: 50697649 | ENST00000414786.6 | SHANK3 | Exon | SCLC-I |
| chr22: 50697652 | ENST00000414786.6 | SHANK3 | Exon | SCLC-I |
| chr22: 50697660 | ENST00000414786.6 | SHANK3 | Exon | SCLC-I |
| chr22: 50697669 | ENST00000414786.6 | SHANK3 | Exon | SCLC-I |
| chr22: 50697682 | ENST00000414786.6 | SHANK3 | Exon | SCLC-I |
| chr22: 50697690 | ENST00000414786.6 | SHANK3 | Exon | SCLC-I |
| chr22: 50697698 | ENST00000414786.6 | SHANK3 | Exon | SCLC-I |
| chr22: 50697700 | ENST00000414786.6 | SHANK3 | Exon | SCLC-I |
| chr22: 50697703 | ENST00000414786.6 | SHANK3 | Exon | SCLC-I |
| chr22: 50697709 | ENST00000414786.6 | SHANK3 | Exon | SCLC-I |
| chr22: 50697725 | ENST00000414786.6 | SHANK3 | Intron | SCLC-I |
| chr22: 50697727 | ENST00000414786.6 | SHANK3 | Intron | SCLC-I |
| chr22: 50697745 | ENST00000414786.6 | SHANK3 | Intron | SCLC-I |
| chr22: 50697751 | ENST00000414786.6 | SHANK3 | Intron | SCLC-I |
| chr22: 50697828 | ENST00000414786.6 | SHANK3 | Intron | SCLC-I |
| chr22: 50697861 | ENST00000414786.6 | SHANK3 | Intron | SCLC-I |
| chr22: 50697894 | ENST00000414786.6 | SHANK3 | Intron | SCLC-I |
| chr22: 50697898 | ENST00000414786.6 | SHANK3 | Intron | SCLC-I |
| chr22: 50697908 | ENST00000414786.6 | SHANK3 | Intron | SCLC-I |
| chr22: 50698101 | ENST00000414786.6 | SHANK3 | Intron | SCLC-I |
| chr3: 72153149 | ENST00000481148.5 | LINC00877 | Intron | SCLC-I |
| chr3: 72153167 | ENST00000481148.5 | LINC00877 | Intron | SCLC-I |
| chr3: 72153179 | ENST00000481148.5 | LINC00877 | Intron | SCLC-I |
| chr3: 72162393 | ENST00000481148.5 | LINC00877 | Intron | SCLC-I |
| chr3: 72162409 | ENST00000481148.5 | LINC00877 | Intron | SCLC-I |
| chr3: 72162418 | ENST00000481148.5 | LINC00877 | Intron | SCLC-I |
| chr3: 125886263 | ENST00000625484.1 | LINC02614 | Intron | SCLC-I |
| chr3: 125886271 | ENST00000625484.1 | LINC02614 | Intron | SCLC-I |
| chr3: 125886293 | ENST00000625484.1 | LINC02614 | Intron | SCLC-I |
| chr3: 186099366 | ENST00000472868.6 | ETV5 | Intron | SCLC-I |
| chr3: 196640762 | ENST00000328557.5 | NRROS | Intron | SCLC-I |
| chr3: 196640920 | ENST00000328557.5 | NRROS | Intron | SCLC-I |
| chr3: 197464061 | ENST00000420213.1 | NA | Intron | SCLC-I |
| chr4: 3308936 | ENST00000506631.5 | RGS12 | Intron | SCLC-I |
| chr4: 3308950 | ENST00000506631.5 | RGS12 | Intron | SCLC-I |
| chr4: 3308952 | ENST00000506631.5 | RGS12 | Intron | SCLC-I |
| chr4: 3308956 | ENST00000506631.5 | RGS12 | Intron | SCLC-I |
| chr4: 3308989 | ENST00000506631.5 | RGS12 | Intron | SCLC-I |
| chr4: 3310192 | ENST00000506631.5 | RGS12 | Intron | SCLC-I |
| chr4: 140092663 | ENST00000502696.1 | MAML3 | Intron | SCLC-I |
| chr4: 140092684 | ENST00000502696.1 | MAML3 | Intron | SCLC-I |
| chr5: 1207091 | ENST00000515652.5 | SLC6A19 | Intron | SCLC-I |
| chr5: 1218443 | ENST00000515652.5 | SLC6A19 | Intron | SCLC-I |
| chr5: 1634751 | ENST00000502273.6 | LOC728613 | Promoter | SCLC-I |
| chr5: 1634775 | ENST00000502273.6 | LOC728613 | Promoter | SCLC-I |
| chr5: 1963148 | ENST00000660942.1 | NA | Intron | SCLC-I |
| chr5: 3599617 | ENST00000559410.1 | NA | Intron | SCLC-I |
| chr5: 3599735 | ENST00000559410.1 | NA | Intron | SCLC-I |
| chr5: 3599748 | ENST00000559410.1 | NA | Intron | SCLC-I |
| chr5: 93570875 | ENST00000656155.1 | NR2F1-AS1 | Intron | SCLC-I |
| chr5: 93570877 | ENST00000656155.1 | NR2F1-AS1 | Intron | SCLC-I |
| chr6: 25179238 | ENST00000643807.1 | CMAHP | Intron | SCLC-I |
| chr6: 25179395 | ENST00000643807.1 | CMAHP | Intron | SCLC-I |
| chr6: 107634349 | ENST00000317357.10 | SOBP | Exon | SCLC-I |
| chr6: 107634371 | ENST00000317357.10 | SOBP | Exon | SCLC-I |
| chr6: 107634379 | ENST00000317357.10 | SOBP | Exon | SCLC-I |
| chr6: 107634382 | ENST00000317357.10 | SOBP | Exon | SCLC-I |
| chr6: 107634391 | ENST00000317357.10 | SOBP | Exon | SCLC-I |
| chr6: 107634400 | ENST00000317357.10 | SOBP | Exon | SCLC-I |
| chr6: 107634406 | ENST00000317357.10 | SOBP | Exon | SCLC-I |
| chr6: 107634416 | ENST00000317357.10 | SOBP | Exon | SCLC-I |
| chr6: 107634421 | ENST00000317357.10 | SOBP | Exon | SCLC-I |
| chr6: 107634428 | ENST00000317357.10 | SOBP | Exon | SCLC-I |
| chr6: 107634434 | ENST00000317357.10 | SOBP | Exon | SCLC-I |
| chr6: 107634650 | ENST00000317357.10 | SOBP | Exon | SCLC-I |
| chr6: 107634671 | ENST00000317357.10 | SOBP | Exon | SCLC-I |
| chr6: 107634681 | ENST00000317357.10 | SOBP | Exon | SCLC-I |
| chr6: 107634776 | ENST00000317357.10 | SOBP | Exon | SCLC-I |
| chr6: 107634797 | ENST00000317357.10 | SOBP | Exon | SCLC-I |
| chr6: 107634800 | ENST00000317357.10 | SOBP | Exon | SCLC-I |
| chr6: 107634806 | ENST00000317357.10 | SOBP | Exon | SCLC-I |
| chr6: 107634815 | ENST00000317357.10 | SOBP | Exon | SCLC-I |
| chr6: 107634825 | ENST00000317357.10 | SOBP | Exon | SCLC-I |
| chr6: 107634885 | ENST00000317357.10 | SOBP | Exon | SCLC-I |
| chr6: 107634987 | ENST00000317357.10 | SOBP | Exon | SCLC-I |
| chr6: 107634992 | ENST00000317357.10 | SOBP | Exon | SCLC-I |
| chr6: 107635008 | ENST00000317357.10 | SOBP | Exon | SCLC-I |
| chr6: 107635014 | ENST00000317357.10 | SOBP | Exon | SCLC-I |
| chr6: 107635027 | ENST00000317357.10 | SOBP | Exon | SCLC-I |
| chr6: 107635032 | ENST00000317357.10 | SOBP | Exon | SCLC-I |
| chr6: 107635047 | ENST00000317357.10 | SOBP | Exon | SCLC-I |
| chr6: 107635056 | ENST00000317357.10 | SOBP | Exon | SCLC-I |
| chr6: 108174645 | ENST00000368986.8 | NR2E1 | Intron | SCLC-I |
| chr6: 108798159 | ENST00000655744.1 | NA | Promoter | SCLC-I |
| chr6: 111551876 | ENST00000532353.1 | TRAF3IP2-AS1 | Intron | SCLC-I |
| chr6: 111551904 | ENST00000532353.1 | TRAF3IP2-AS1 | Intron | SCLC-I |
| chr6: 111552096 | ENST00000532353.1 | TRAF3IP2-AS1 | Intron | SCLC-I |
| chr7: 45028581 | ENST00000461377.5 | CCM2 | Intron | SCLC-I |
| chr7: 45028613 | ENST00000461377.5 | CCM2 | Intron | SCLC-I |
| chr7: 45768094 | ENST00000429741.5 | SEPTIN7P2 | Intron | SCLC-I |
| chr7: 45768098 | ENST00000429741.5 | SEPTIN7P2 | Intron | SCLC-I |
| chr7: 78040283 | ENST00000354212.9 | MAGI2 | Intron | SCLC-I |
| chr7: 78040332 | ENST00000354212.9 | MAGI2 | Intron | SCLC-I |
| chr7: 78040354 | ENST00000354212.9 | MAGI2 | Intron | SCLC-I |
| chr7: 78040430 | ENST00000354212.9 | MAGI2 | Intron | SCLC-I |
| chr7: 78040432 | ENST00000354212.9 | MAGI2 | Intron | SCLC-I |
| chr7: 101232397 | ENST00000308344.9 | CLDN15 | Exon | SCLC-I |
| chr7: 101232416 | ENST00000308344.9 | CLDN15 | Exon | SCLC-I |
| chr7: 101232422 | ENST00000308344.9 | CLDN15 | Exon | SCLC-I |
| chr7: 101232431 | ENST00000308344.9 | CLDN15 | Exon | SCLC-I |
| chr7: 101232465 | ENST00000308344.9 | CLDN15 | Exon | SCLC-I |
| chr7: 101232485 | ENST00000308344.9 | CLDN15 | Exon | SCLC-I |
| chr7: 101232508 | ENST00000308344.9 | CLDN15 | Exon | SCLC-I |
| chr7: 101232511 | ENST00000308344.9 | CLDN15 | Exon | SCLC-I |
| chr7: 150958216 | ENST00000262186.10 | KCNH2 | Exon | SCLC-I |
| chr7: 150958354 | ENST00000262186.10 | KCNH2 | Exon | SCLC-I |
| chr7: 150958396 | ENST00000262186.10 | KCNH2 | Exon | SCLC-I |
| chr7: 150958403 | ENST00000262186.10 | KCNH2 | Exon | SCLC-I |
| chr8: 8892326 | ENST00000276282.7 | MFHAS1 | Exon | SCLC-I |
| chr8: 8892330 | ENST00000276282.7 | MFHAS1 | Exon | SCLC-I |
| chr8: 8892336 | ENST00000276282.7 | MFHAS1 | Exon | SCLC-I |
| chr8: 8892337 | ENST00000276282.7 | MFHAS1 | Exon | SCLC-I |
| chr8: 8892347 | ENST00000276282.7 | MFHAS1 | Exon | SCLC-I |
| chr8: 8892409 | ENST00000276282.7 | MFHAS1 | Exon | SCLC-I |
| chr8: 8892410 | ENST00000276282.7 | MFHAS1 | Exon | SCLC-I |
| chr8: 8892416 | ENST00000276282.7 | MFHAS1 | Exon | SCLC-I |
| chr8: 11474156 | ENST00000527445.1 | FAM167A | Intron | SCLC-I |
| chr8: 11474157 | ENST00000527445.1 | FAM167A | Intron | SCLC-I |
| chr8: 11474164 | ENST00000527445.1 | FAM167A | Intron | SCLC-I |
| chr8: 11474171 | ENST00000527445.1 | FAM167A | Intron | SCLC-I |
| chr8: 22643830 | ENST00000519513.5 | BIN3 | Intron | SCLC-I |
| chr8: 22643945 | ENST00000519513.5 | BIN3 | Intron | SCLC-I |
| chr8: 60946896 | ENST00000526936.5 | NA | Intron | SCLC-I |
| chr8: 60946919 | ENST00000526936.5 | NA | Intron | SCLC-I |
| chr8: 144467777 | ENST00000529644.1 | KIFC2 | Exon | SCLC-I |
| chr9: 34457430 | ENST00000379080.5 | FAM219A | Intron | SCLC-I |
| chr9: 34457464 | ENST00000379080.5 | FAM219A | Intron | SCLC-I |
| chr9: 113400746 | ENST00000464749.5 | ALAD | Intron | SCLC-I |
| chr9: 113463513 | ENST00000374140.6 | RGS3 | Intron | SCLC-I |
| chr9: 113463523 | ENST00000374140.6 | RGS3 | Intron | SCLC-I |
| chr9: 113463527 | ENST00000374140.6 | RGS3 | Intron | SCLC-I |
| chr9: 113463528 | ENST00000374140.6 | RGS3 | Intron | SCLC-I |
| chr9: 113463555 | ENST00000374140.6 | RGS3 | Intron | SCLC-I |
| chr9: 113463557 | ENST00000374140.6 | RGS3 | Intron | SCLC-I |
| chr9: 113463558 | ENST00000374140.6 | RGS3 | Intron | SCLC-I |
| chr9: 113463567 | ENST00000374140.6 | RGS3 | Intron | SCLC-I |
| chr9: 113463568 | ENST00000374140.6 | RGS3 | Intron | SCLC-I |
| chr9: 113463569 | ENST00000374140.6 | RGS3 | Intron | SCLC-I |
| chr9: 113463570 | ENST00000374140.6 | RGS3 | Intron | SCLC-I |
| chr9: 113463574 | ENST00000374140.6 | RGS3 | Intron | SCLC-I |
| chr9: 113463575 | ENST00000374140.6 | RGS3 | Intron | SCLC-I |
| chr9: 113463578 | ENST00000374140.6 | RGS3 | Intron | SCLC-I |
| chr9: 113463581 | ENST00000374140.6 | RGS3 | Intron | SCLC-I |
| chr9: 113463598 | ENST00000374140.6 | RGS3 | Intron | SCLC-I |
| chr9: 127754240 | ENST00000468969.6 | SH2D3C | Intron | SCLC-I |
| chr9: 127754243 | ENST00000468969.6 | SH2D3C | Intron | SCLC-I |
| chr9: 127754244 | ENST00000468969.6 | SH2D3C | Intron | SCLC-I |
| chr9: 127754260 | ENST00000468969.6 | SH2D3C | Intron | SCLC-I |
| chr9: 127754261 | ENST00000468969.6 | SH2D3C | Intron | SCLC-I |
| chr9: 127754267 | ENST00000468969.6 | SH2D3C | Intron | SCLC-I |
| chr9: 127754268 | ENST00000468969.6 | SH2D3C | Intron | SCLC-I |
| chr9: 127754269 | ENST00000468969.6 | SH2D3C | Intron | SCLC-I |
| chr9: 127754270 | ENST00000468969.6 | SH2D3C | Intron | SCLC-I |
| chr9: 127754273 | ENST00000468969.6 | SH2D3C | Intron | SCLC-I |
| chr9: 127754274 | ENST00000468969.6 | SH2D3C | Intron | SCLC-I |
| chr9: 127754294 | ENST00000468969.6 | SH2D3C | Intron | SCLC-I |
| chr9: 127754301 | ENST00000468969.6 | SH2D3C | Intron | SCLC-I |
| chr9: 127754302 | ENST00000468969.6 | SH2D3C | Intron | SCLC-I |
| chr9: 127754304 | ENST00000468969.6 | SH2D3C | Intron | SCLC-I |
| chr9: 127754305 | ENST00000468969.6 | SH2D3C | Intron | SCLC-I |
| chr9: 127754307 | ENST00000468969.6 | SH2D3C | Intron | SCLC-I |
| chr9: 127754342 | ENST00000468969.6 | SH2D3C | Intron | SCLC-I |
| chr9: 127754343 | ENST00000468969.6 | SH2D3C | Intron | SCLC-I |
| chr9: 127754345 | ENST00000468969.6 | SH2D3C | Intron | SCLC-I |
| chr9: 127754355 | ENST00000468969.6 | SH2D3C | Intron | SCLC-I |
| chr9: 127754649 | ENST00000468969.6 | SH2D3C | Intron | SCLC-I |
| chr9: 127754836 | ENST00000468969.6 | SH2D3C | Intron | SCLC-I |
| chr9: 127754846 | ENST00000468969.6 | SH2D3C | Intron | SCLC-I |
| chr9: 127754871 | ENST00000468969.6 | SH2D3C | Intron | SCLC-I |
| chr9: 127754889 | ENST00000468969.6 | SH2D3C | Intron | SCLC-I |
| chr9: 127754894 | ENST00000468969.6 | SH2D3C | Intron | SCLC-I |
| chr9: 127755283 | ENST00000468969.6 | SH2D3C | Intron | SCLC-I |
| chr9: 127755336 | ENST00000468969.6 | SH2D3C | Intron | SCLC-I |
| chr9: 127755497 | ENST00000468969.6 | SH2D3C | Intron | SCLC-I |
| chr9: 127755519 | ENST00000468969.6 | SH2D3C | Intron | SCLC-I |
| chr9: 127755545 | ENST00000468969.6 | SH2D3C | Intron | SCLC-I |
| chr9: 127755548 | ENST00000468969.6 | SH2D3C | Intron | SCLC-I |
| chr9: 127755564 | ENST00000468969.6 | SH2D3C | Intron | SCLC-I |
| chr9: 129495716 | ENST00000653047.1 | LINC00963 | Intron | SCLC-I |
| chr9: 129495789 | ENST00000653047.1 | LINC00963 | Intron | SCLC-I |
| chr9: 131554650 | ENST00000636672.1 | PRRT1B | Exon | SCLC-I |
| chr9: 131554658 | ENST00000636672.1 | PRRT1B | Exon | SCLC-I |
| chr9: 131554659 | ENST00000636672.1 | PRRT1B | Exon | SCLC-I |
| chr9: 131554660 | ENST00000636672.1 | PRRT1B | Exon | SCLC-I |
| chr9: 131554665 | ENST00000636672.1 | PRRT1B | Exon | SCLC-I |
| chr9: 131554666 | ENST00000636672.1 | PRRT1B | Exon | SCLC-I |
| chr9: 131554674 | ENST00000636672.1 | PRRT1B | Exon | SCLC-I |
| chr9: 131554676 | ENST00000636672.1 | PRRT1B | Exon | SCLC-I |
| chr9: 131554679 | ENST00000636672.1 | PRRT1B | Exon | SCLC-I |
| chr9: 131554683 | ENST00000636672.1 | PRRT1B | Exon | SCLC-I |
| chr9: 131554685 | ENST00000636672.1 | PRRT1B | Exon | SCLC-I |
| chr9: 131554696 | ENST00000636672.1 | PRRT1B | Exon | SCLC-I |
| chr9: 131554702 | ENST00000636672.1 | PRRT1B | Exon | SCLC-I |
| chr9: 131554707 | ENST00000636672.1 | PRRT1B | Exon | SCLC-I |
| chr9: 131554714 | ENST00000636672.1 | PRRT1B | Exon | SCLC-I |
| chr9: 131554750 | ENST00000636672.1 | PRRT1B | Exon | SCLC-I |
| chr9: 131885496 | ENST00000357028.6 | MED27 | Intron | SCLC-I |
| chr9: 132037677 | ENST00000651950.1 | MED27 | Intron | SCLC-I |
| chr9: 136076236 | ENST00000277554.4 | NACC2 | Intron | SCLC-I |
| chr9: 136076244 | ENST00000277554.4 | NACC2 | Intron | SCLC-I |
| chr9: 136076251 | ENST00000277554.4 | NACC2 | Intron | SCLC-I |

### Example 6—Methylation Markers for Analysis of Tumor Burden

Whole genome sequencing and methylation analysis data was obtained from cell free DNA (cfDNA) from SCLC patients. 42 methylation sites were identified that correlated well with general tumor burden in the patients. These methylation sites are provided in Table 21.

TABLE 24
Methylation sites having methylation levels
correlated with general tumor burden in SCLC
Methylation Site

chr5: 77844815
chr5: 77844832
chr5: 77844821
chr5: 132257679
chr16: 49280789
chr21: 34670607
chr21: 34670604
chr21: 34670609
chr6: 108176627
chr10: 71638792
chr5: 132257648
chr5: 132257653
chr5: 132257685
chr5: 132257670
chr5: 138274351
chr8: 22139065
chr5: 132257664
chr5: 132257652
chr5: 132257666
chr3: 38039198
chr9: 137591221
chr9: 131444459
chr6: 108176609
chr5: 132257661
chr5: 132257650
chr5: 132257703
chr5: 132257682
chr5: 178860732
chr5: 132257676
chr20: 22582930
chr2: 10043081
chr2: 98347974
chr5: 132257649
chr5: 178860742
chr5: 138274357
chr5: 138274360
chr10: 75408778
chr5: 132257647
chr5: 132257637
chr6: 108176612
chr22: 50251762
chr21: 34669575

### Example 7—Tumor- and Circulating-Free DNA Methylation Identifies Clinically Relevant Small Cell Lung Cancer Subtypes

Here, the inventors assessed DNA methylation and gene expression from a cohort of predominantly extensive stage SCLC with tissue and/or plasma samples and developed machine learning approaches to allow the classification of SCLC subtypes from clinical specimen in both tissue and liquid biopsies in order to identify SCLC subgroups and enable precision medicine in SCLC.

Cohort of Clinical Specimens for RNAseq and DNA Methylation Profiling

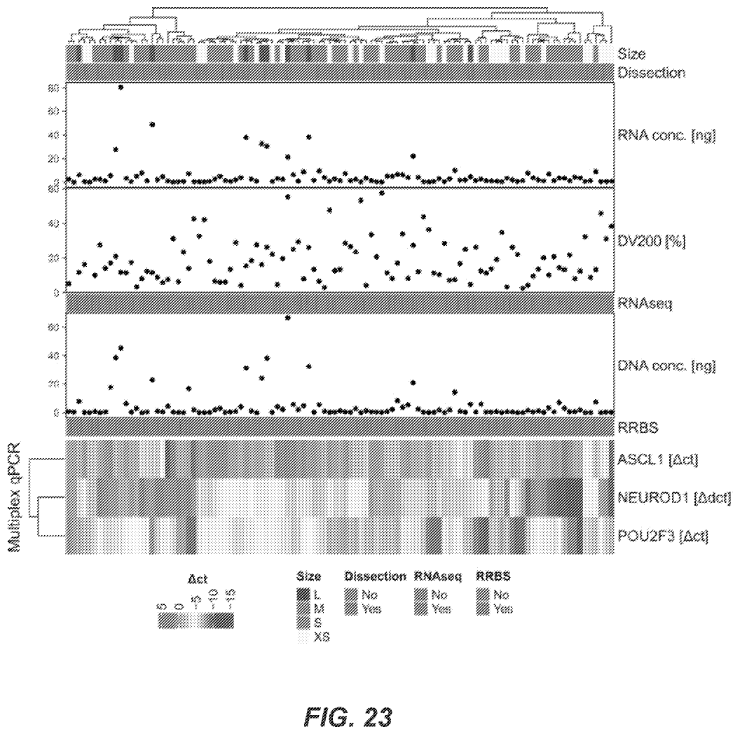

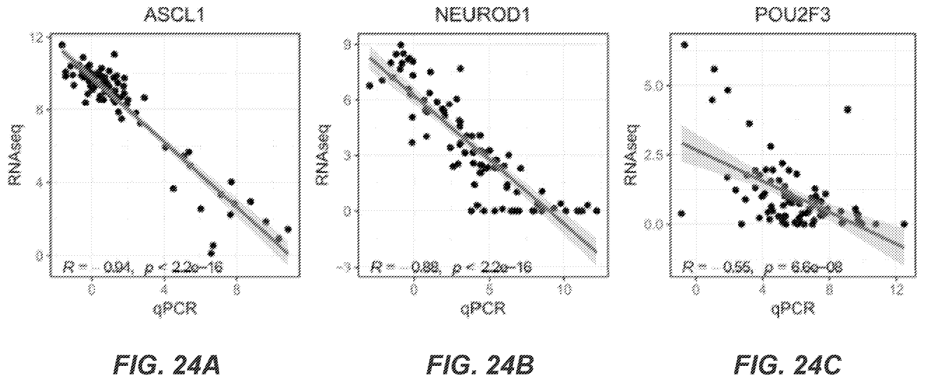

Given the finding that DNA methylation was able to detect SCLC from plasma, the inventors next hypothesized that DNA methylation can be exploited as a biomarker in SCLC. To this end the inventors investigated a cohort of 105 samples with predominantly extensive-stage SCLC (Table 1). For 85 samples the inventors obtained RNAseq data and for 83 samples the inventors obtained DNA methylation data using reduced representation bisulfite sequencing (RRBS), with 66 samples having both, RNAseq and RRBS data. The three groups were balanced among sex, stage and subtypes (Table 25). Further data on the cohort is summarized in FIG. 23. To further confirm the subgroup identity and the reliability of the RNAseq data, results were compared to qPCR of the three transcription factors (ASCL1. NEUROD1 and POU2F3) to ensure reliability. The inventors observed a high correlation with R=−0.94, R=−0.88, R=−0.55, respectively (FIGS. 24A-24C).

TABLE 25

| | | | | | | | | Chi- |
|---|---|---|---|---|---|---|---|---|
| | | All | | RNAseq | | RRBS | | sq p |
| N | | 105 | | 85 | | 83 | | |
| Age | | 66 | (26-96) | 66 | (28-96) | 66 | (26-89) | |
| (range) | | | | | | | | |
| Sex (%) | F | 57 | (54%) | 45 | (53%) | 46 | (55%) | 0.949 |
| | M | 48 | (46%) | 40 | (47%) | 37 | (45%) | |
| Stage (%) | LS | 28 | (27%) | 25 | (29%) | 22 | (27%) | 0.890 |
| | ES | 77 | (73%) | 60 | (71%) | 61 | (73%) | |
| Subtype | SCLC-A | 66 | (63%) | 49 | (58%) | 57 | (60%) | 0.863 |
| (%) | SCLC-N | 22 | (21%) | 22 | (26%) | 19 | (24%) | |
| | SCLC-P | 4 | (4%) | 4 | (5%) | 3 | (4%) | |
| | SCLC-I | 8 | (8%) | 8 | (9%) | 4 | (5%) | |
| | equivocal | 2 | (2%) | 2 | (2%) | 0 | (6%) | |
| | unknown | 3 | (3%) | 0 | (0%) | 0 | (0%) | |

Clinical SCLC can be Classified Using a Reduced Machine Learning RNAseq Signature

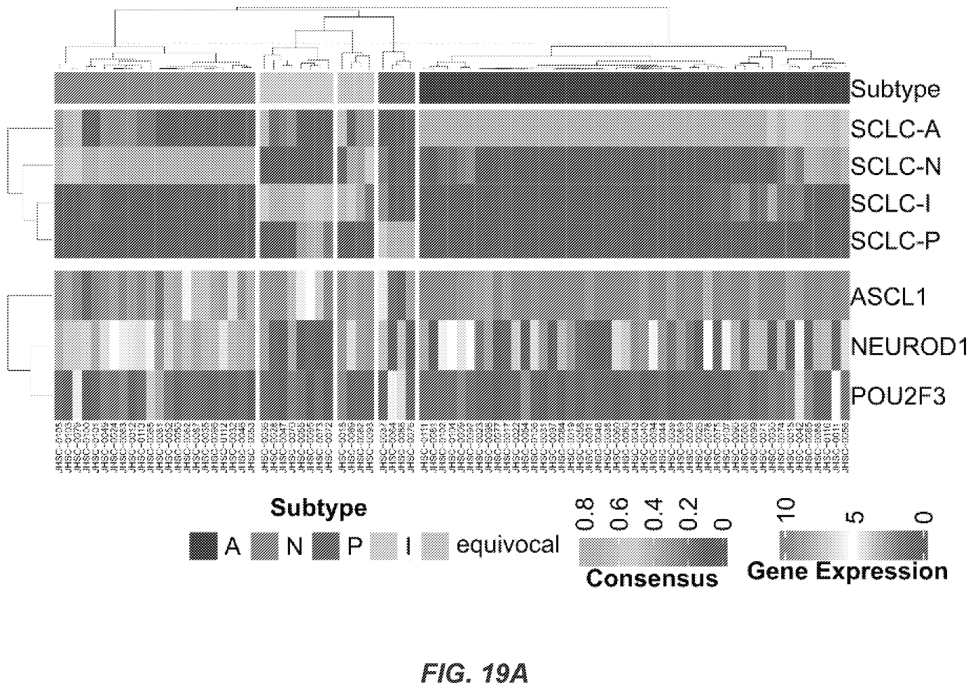

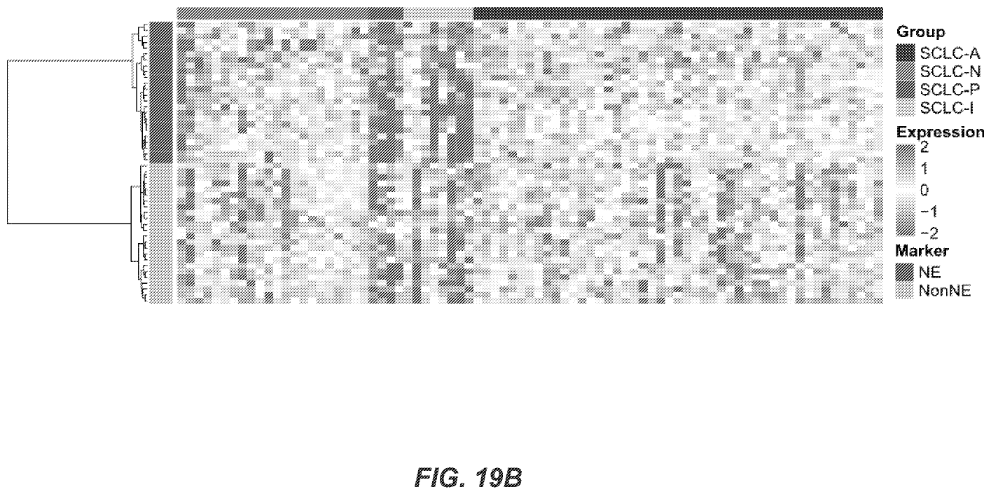

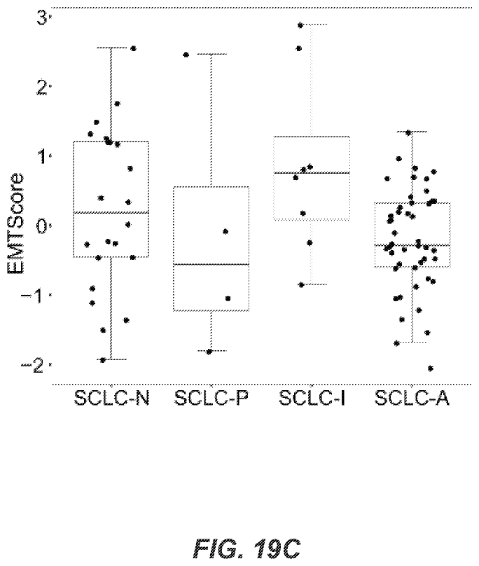

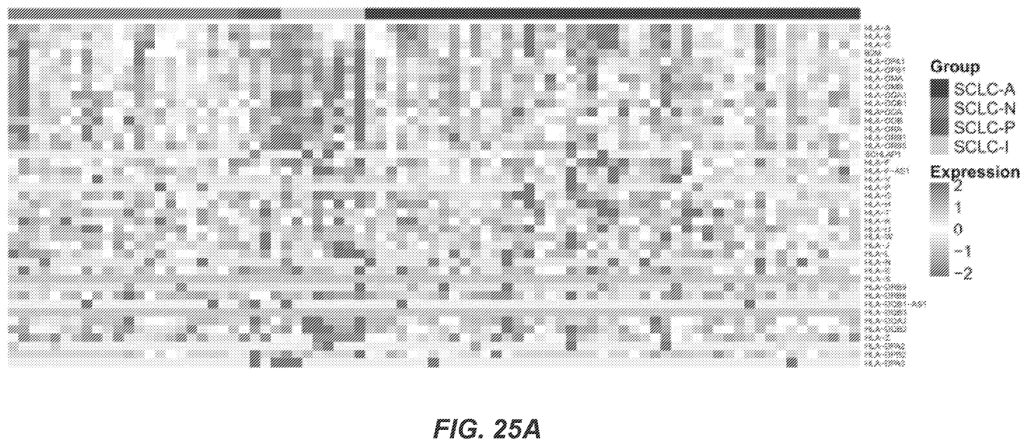

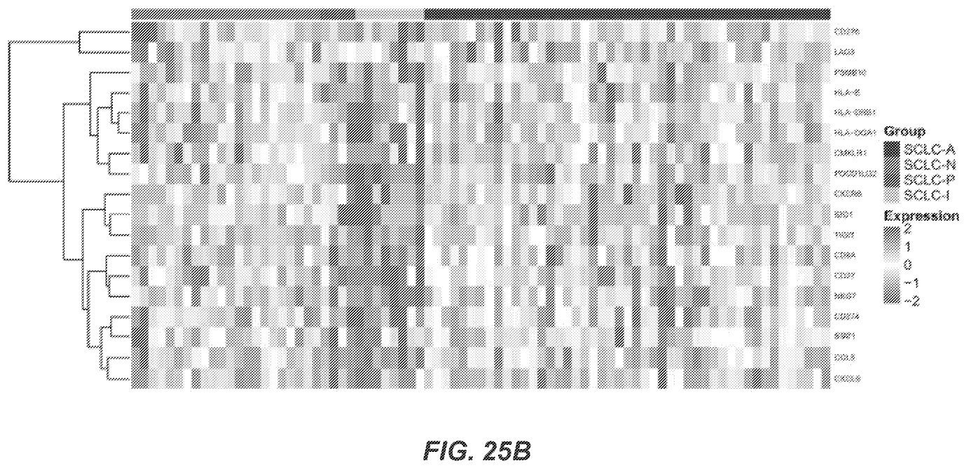

The inventors previously reported that SCLC can be classified in four distinct subtypes using a gene expression classifier derived from non-negative matrix factorization¹¹ from using mRNA expression data from a cohort 20 of limited stage SCLC surgical specimens and the IMPower133 dataset from a randomized phase 3 clinical trial assessing the combination of first-line platinum-etoposide chemotherapy with or without atezolizumab², comprised of extensive stage SCLC specimens. Building on this analysis, the inventors developed a classifier in order to reduce the number of genes required to subtype tumors and facilitate the subtype classification using different mRNA profiling methods. Using a consensus classification (see online methods) incorporating 181 genes, the inventors were able to unambiguously classify the majority of samples into a single subtype, and expression of the previously established transcription factors (ASCL1 for SCLC-A. NEUROD1 for SCLC-N and POU2F3 for SCLC-P) was well correlated with the classification (FIG. 19A). For 81/85 samples, a clear classification was obtained, with 47/81 (58%), 22/81 (27%), 4/810 (5%), 8/81 (10%) representing the SCLC-A, SCLC-N, SCLC-P and SCLC-I subtypes, respectively. This distribution is close to the observed distribution in the IMpower133 study with SCLC-A—51%, SCLC-N—23%. SCLC-I—18%. SCLC-P—7%¹¹ (chi-sq p=0.2116). As previously published, the SCLC-A and SCLC-N samples in the cohort demonstrated a higher expression of neuroendocrine genes compared to SCLC-P and SCLC-I (FIG. 19B) while the latter was also characterized by a more mesenchymal polarization (FIG. 19C; ANOVA p=0.032). Likewise, expression of HLA and other immune-related genes was higher in SCLC-P and SCLC-I subtype (FIGS. 25A and 25B), providing evidence that the classification is valid and the phenotypes consistent between the datasets. Only few specimens failed classification (4/81: 4.9%) due to what appeared to be technical limitations and RNA quality (FIG. 19B). Consequently, with a success rate of 95%, the classification approach is highly robust, comprises of only a limited number of 181 genes whose assessment is technically less challenging than larger gene panels, and clearly allows routine classification.

DNA Methylation Varies Across SCLC Subtypes

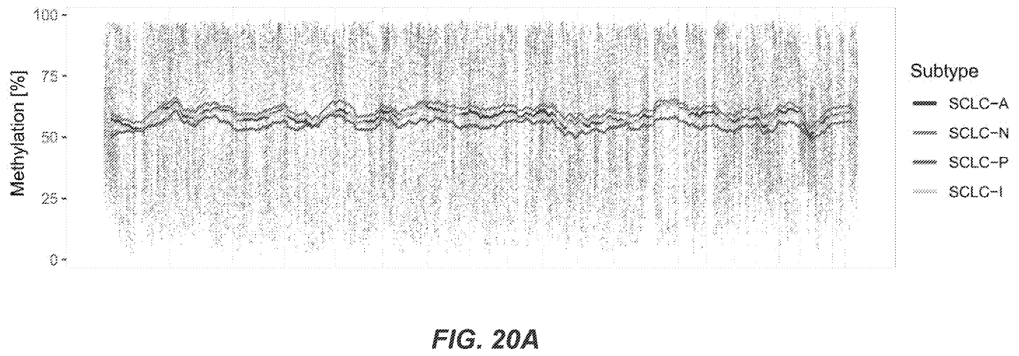

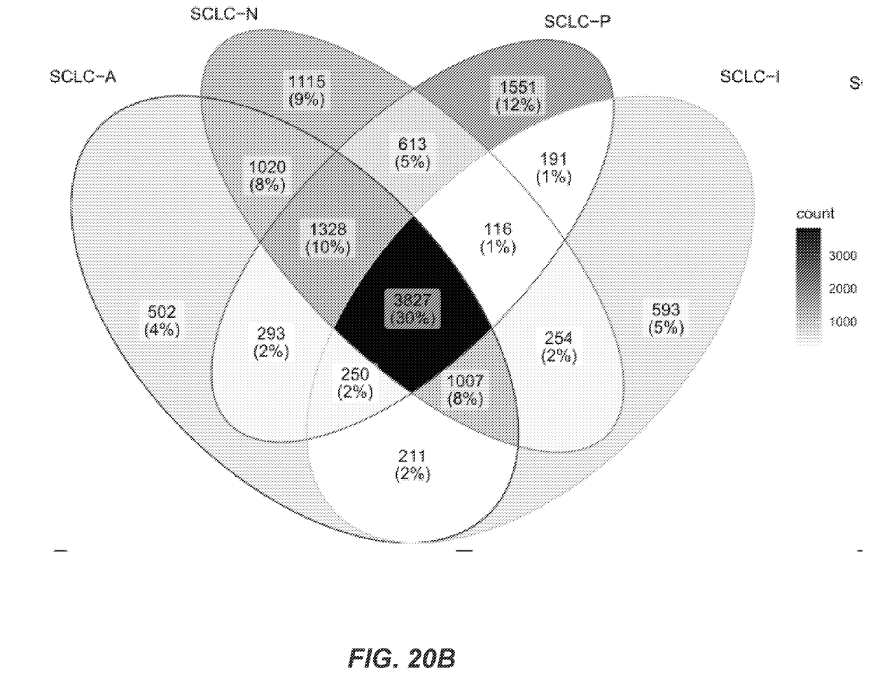

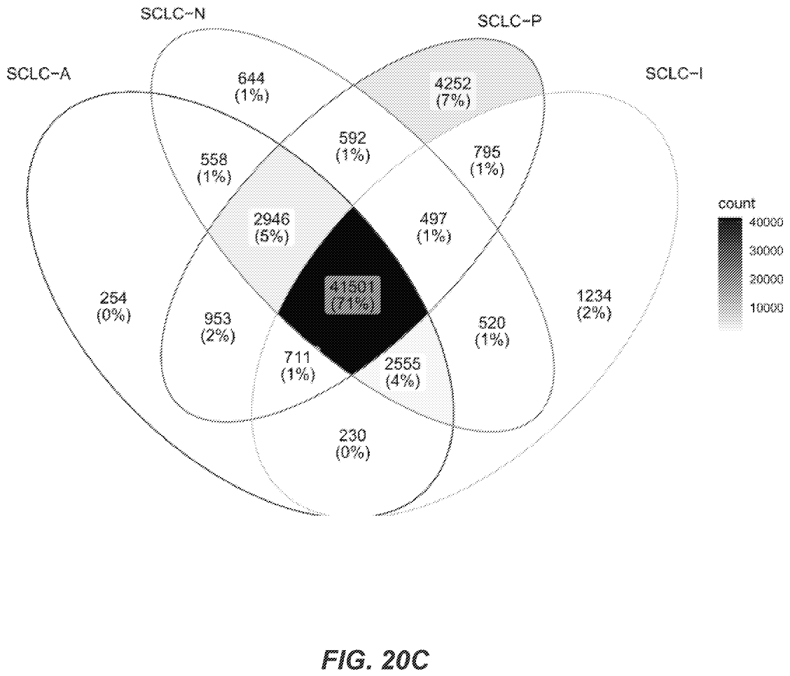

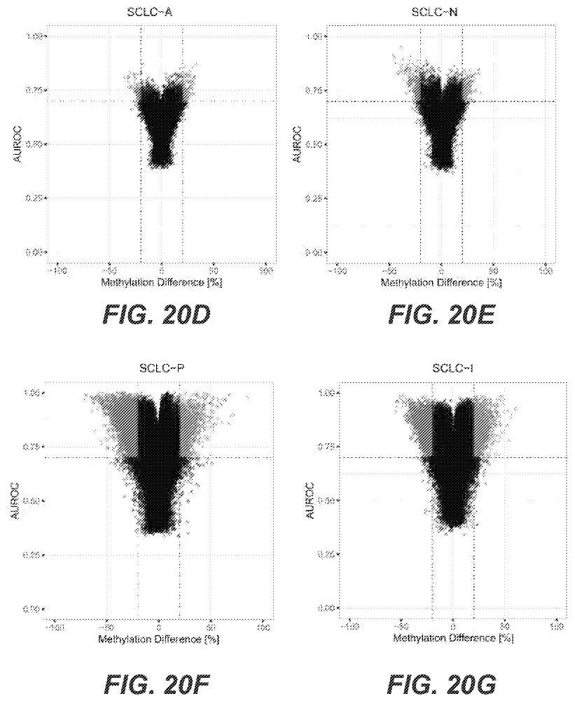

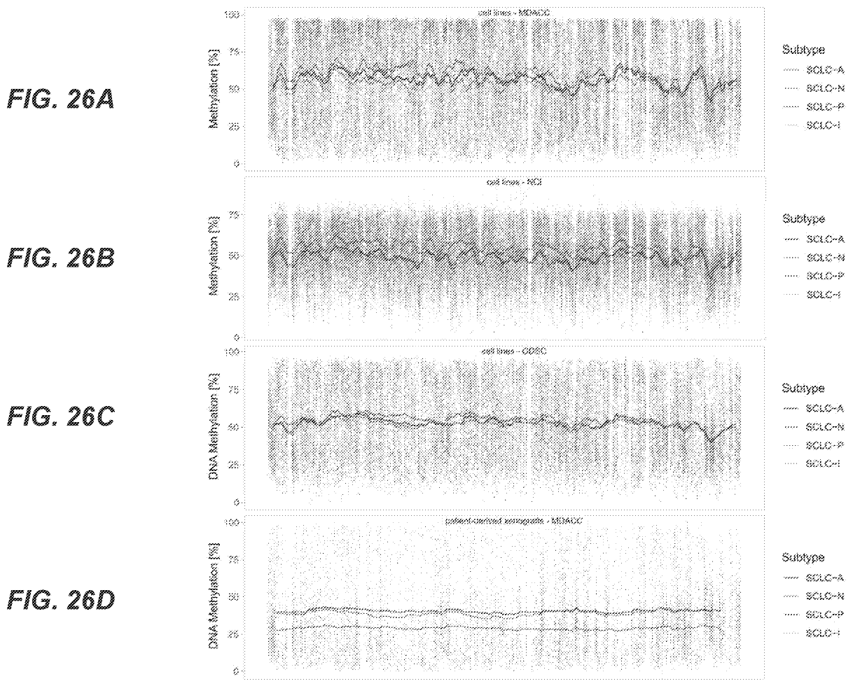

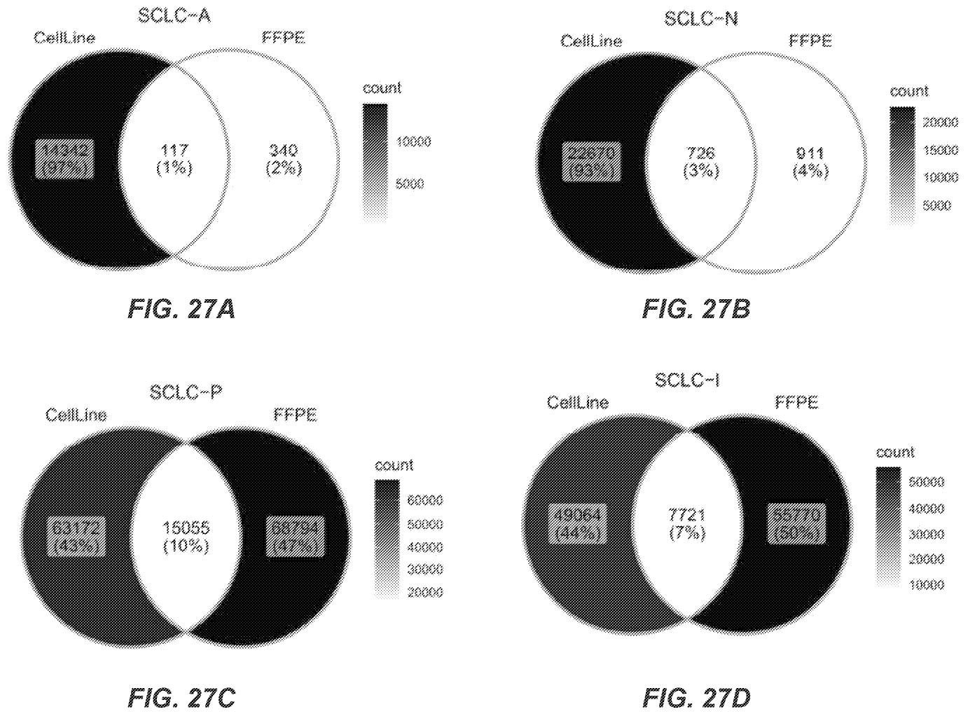

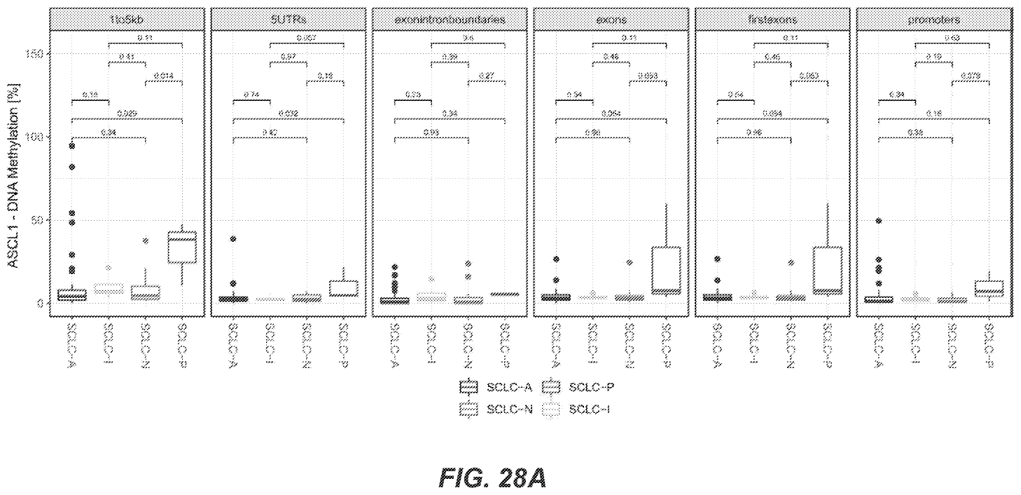

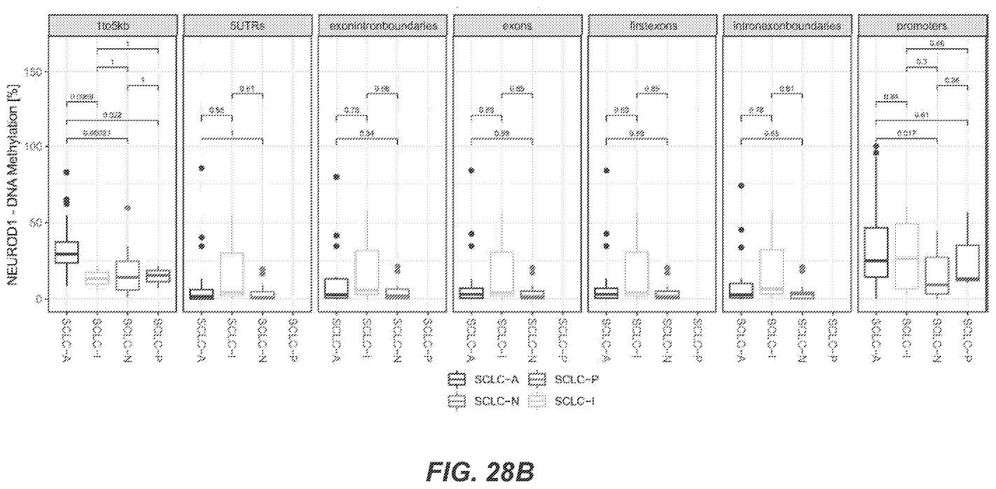

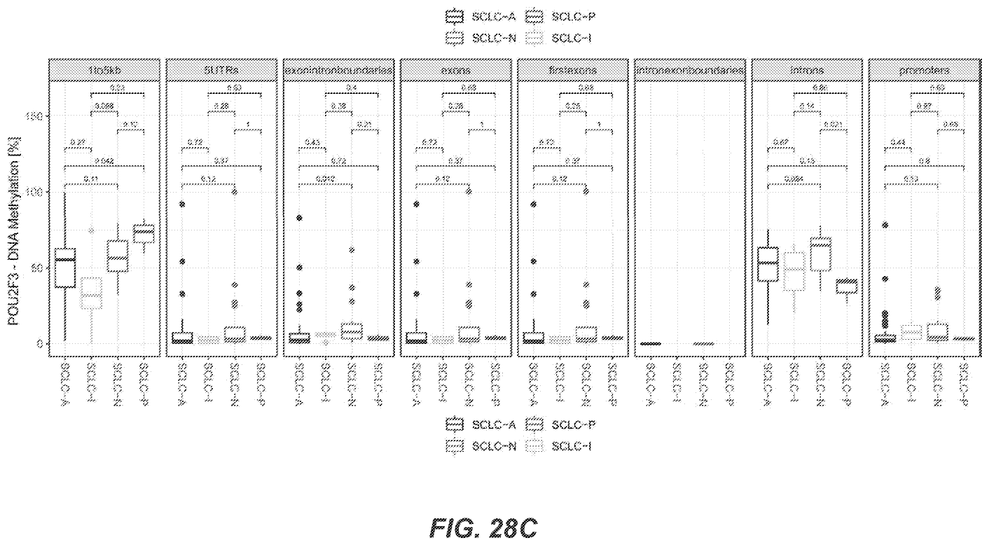

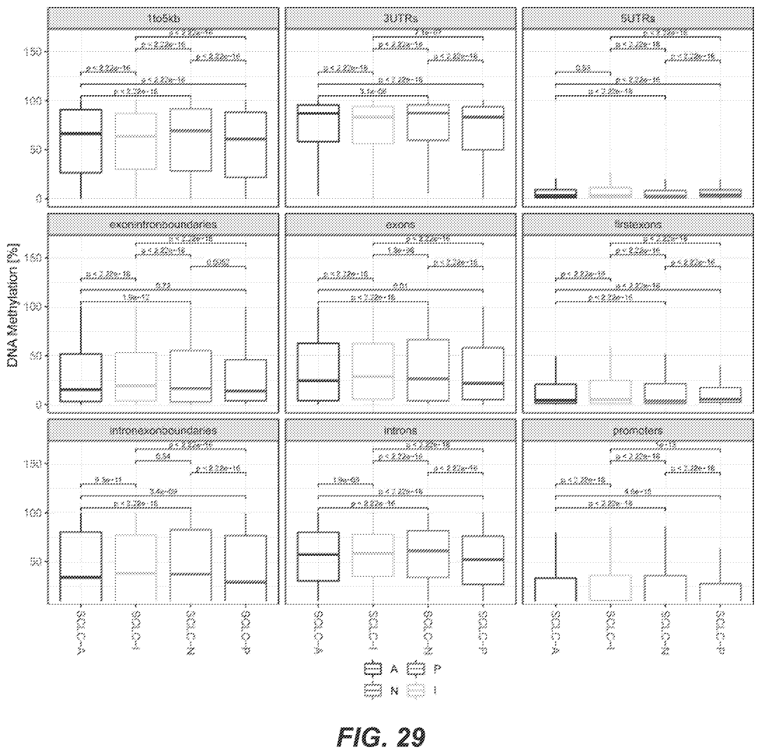

The inventors then analyzed the differences of DNA methylation in the dataset. The methylation level was averaged across bins of 100 kb width and calculated the mean for those bins per subtype (FIG. 20A). To determine the genome-wide methylation level, the rolling average was calculated over 500 bins (=50 Mbp). The analysis highlighted profound differences in the global methylation level per subtype, with the SCLC-P subtype presenting with a hypomethylated phenotype and SCLC-N with a hypermethylated phenotype, while SCLC-A and SCLC-I were comparable (FIG. 20A). The inventors further analyzed 59 SCLC-derived cell lines across all four subtypes as well as 12 patient-derived xenograft models that span all but the SCLC-I phenotype, together with two previously published datasets on cell lines. Interestingly, in cell lines, SCLC-P was hypermethylated (FIG. 26A) which was confirmed in two independent datasets of cell lines from the NCI SCLC cell miner project²¹ (FIG. 26B) and the GDSC²² (FIG. 26C), while the xenograft models confirmed the hypomethylated phenotype in SCLC-P (FIG. 26D). The inventors analyzed the association of single DNA methylation sites in FFPE samples and cell lines using receiver operator characteristics and filtered for methylation sites that were present in both FFPE samples and cell lines. When comparing DNA methylation sites that were highly associated with one of the subtypes (AUROC >0.8), only few were shared among the two datasets even though some DNA methylation sites were highly associated with the respective subtype in both datasets (FIG. 27A-27D), highlighting that cell lines and FFPE samples share some similarities despite the genome-wide differences. The inventors further annotated the methylation sites by their association with genes and compared the differences. The inventors selected the hypermethylated regions (FIG. 20B), defined by >90% methylation level, and the hypomethylated regions (FIG. 20C), defined by <10% methylation level. Interestingly, most of those regions were conserved across the subtypes and only a few regions were specific to each. The inventors further analyzed the association of each region with one of the subtypes using receiver operator characteristics and analyzed regions with +/−20% differences of the subtype versus the other subtypes for SCLC-A (FIG. 20D), SCLC-N(FIG. 20E), SCLC-P (FIG. 20F) and SCLC-I (FIG. 20G). Importantly, while only few regions were specific to SCLC-A and SCLC-N, the inventors saw many regions that were specific to the SCLC-P and SCLC-I subtypes, respectively, which revealed that those subtypes harbor more specific methylated regions. As the four subtypes are defined by the expression of the three transcription factors, ASCL1, NEUROD1 and POU2F3, the inventors analyzed the methylation in those genes between the subtypes (FIG. 28A-28C). The inventors have previously demonstrated in a comparison of cell lines of the SCLC-A and SCLC-N subtype, that NEUROD1 promoter methylation is reduced in SCLC-N and confirmed this finding in tumor specimen (FIG. 28B). Other than this case, however, methylation in regions associated with those genes did not differ strongly across the subtypes. Consequently, the regulation of these specific transcription factors by DNA methylation might be limited and consequently analysis of only those regions does not appear to be adequate to differentiate between SCLC subtypes. Nevertheless, the inventors saw marked differences in the average methylation level across specific regions (FIG. 29). Consequently, profound differences in the DNA methylation composition between the four subtypes confirmed the pivotal role of epigenetic regulation in SCLC.

DNA Methylation can be Used to Classify SCLC Specimen

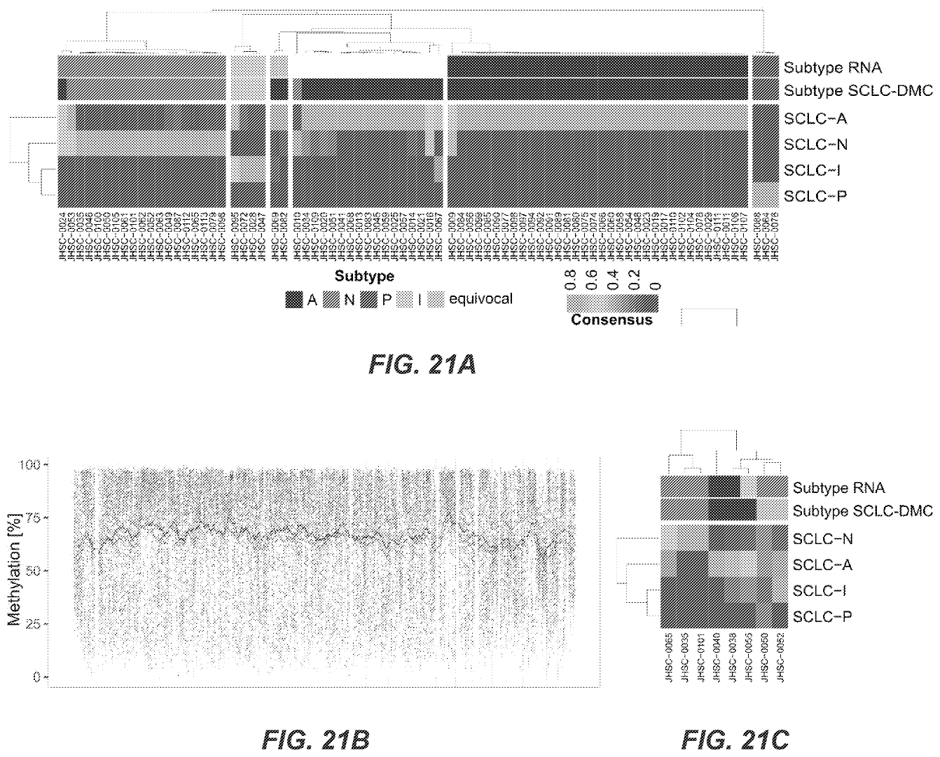

These findings suggested that differences in DNA methylation could be exploited for the generation of biomarkers that are able to differentiate SCLC subtypes. In order to first generate models that work across different datasets and to capture the variability across SCLC, the inventors combined data from the clinical tumor specimens and cell lines to define DNA methylation sites that are associated with each of the four subtypes in both datasets using ROC. The inventors then selected the top 500 sites for each of the four subtypes and created models that were trained only on the clinical tumor specimens by randomly selecting 5, 10, or 20 methylation sites per subtype. The inventors selected for models that had an accuracy=100% and used a consensus of 50% across the models to call a subtype, similar to the RNAseq approach (FIG. 21A). Accuracy for samples that have been associated with one of the four subtypes using RNAseq and DNA methylation was 98.3% (95% CI: 91.1%-99.9%; Kappa=0.9706) with the DNA methylation-based classifier (SCLC-DMC). Importantly, the SCLC-DMC approach allowed the subtyping of 17 additional samples for which no RNAseq data was available and thus RNA-based classification was impossible. Furthermore, for 2 samples with equivocal classification in RNAseq, the SCLC-DMC allowed the association of a subtype (FIG. 21A).

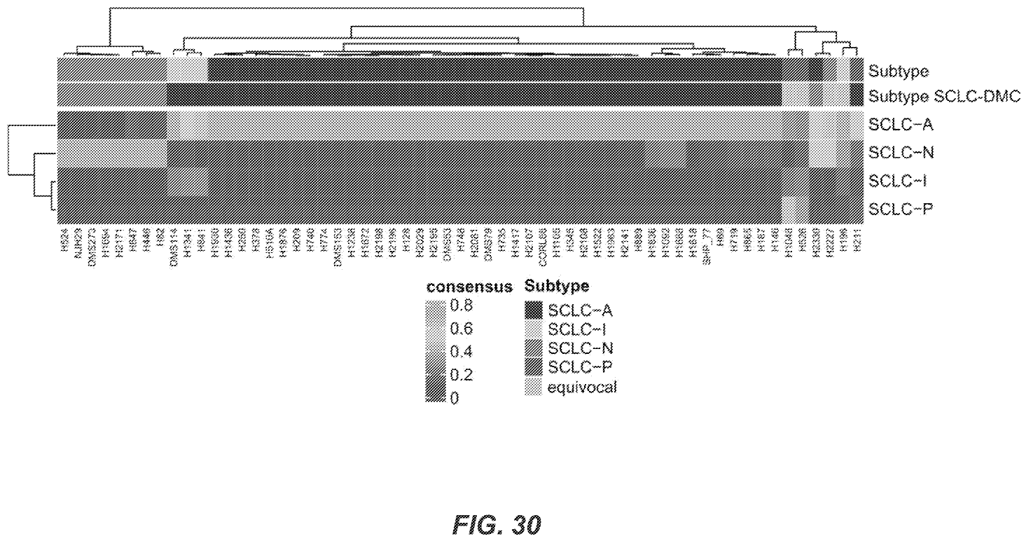

Prior studies indicated that the SCLC subtypes differ in terms of their tumor microenvironment; for example, the SCLC-I subgroup was more inflamed with enrichment for immune-related checkpoint factors and likewise in SCLC-P, tumor microenvironment is enriched. It was therefore expected that cell lines could not fully recapitulate differences between the subgroups observed using clinical specimens. To test this, the inventors investigated how the SCLC-DMC on the classification of cell lines. While the accuracy was high (accuracy=90.9%; 95% CI: 80.0%-97.0%), the SCLC-DMC failed to classify SCLC-P and SCLC-I (FIG. 30). While this highlights that DNA methylation sites are conserved to a significant extent between cell lines and tumor samples, it further demonstrates the limitations in relying on exclusively on cell line data which is lacking the tumor microenvironment that is especially important in those two subtypes where classification failed, and thus limits its classification. Crucially, despite limited capabilities in cell lines, the SCLC-DMC was highly reliable and precise in defining subtypes in clinical SCLC samples.

DNA Methylation is Preserved in cfDNA

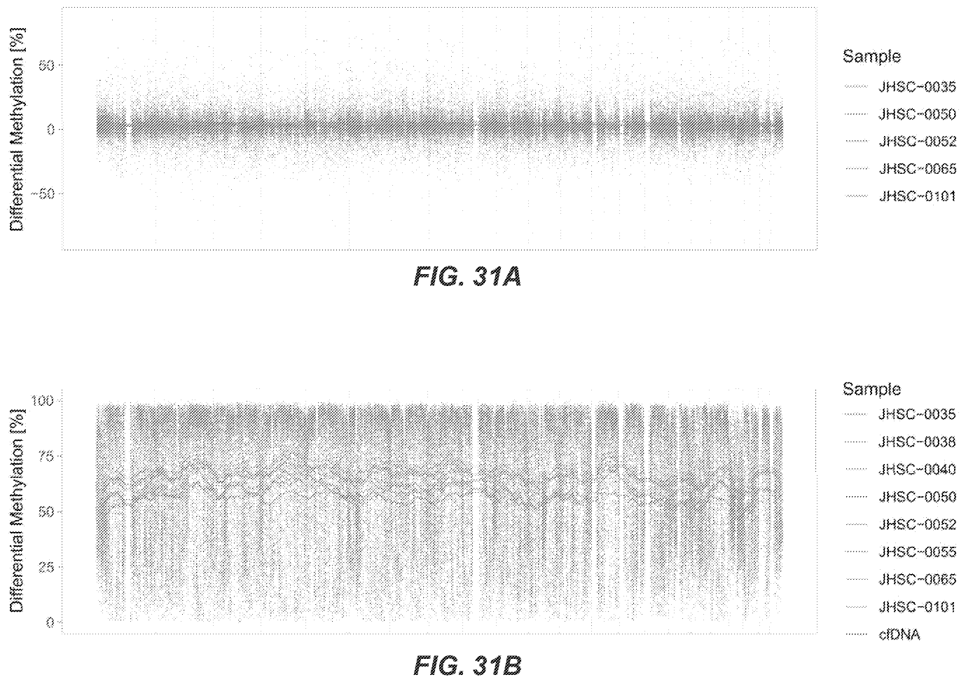

As DNA methylation was highly associated with SCLC subtypes in the dataset, the inventors further hypothesized that DNA methylation from plasma might equally serve for the classification of SCLC. The inventors consequently analyzed DNA methylation in 8 matched plasma samples (of which the inventors had matched FFPE RRBS for 5), which covered all but the SCLC-P subtype. Importantly, the DNA methylation profile across the genome was comparable to the FFPE samples (FIG. 21B). Furthermore, the inventors analyzed the differences for each sample between the cfDNA and FFPE DNA methylation data (FIG. 31A) and observed that differences between the DNA sources were minor and that DNA methylation was preserved between FFPE and cfDNA. Additionally, the inventors retrieved previously published cfRRBS data from healthy donors²³ to use as control samples. The profile of all samples was comparable to the healthy donor cfDNA profile (FIG. 31B), with the exception of JHSC-0050 where the profile differed markedly. The classification of SCLC subtypes from the cfDNA was performed using the SCLC-DMC that was trained on clinical FFPE specimens and was not further adjusted for the cfDNA input. Nevertheless, classification was possible for most samples (83.3%, 95% CI: 35.88%-99.6%; Kappa=0.7143) with all but one subtype being correctly classified (FIG. 21C). However, two samples failed classification including JHSC-0050 which had an abnormal cfDNA pattern and thus it is feasible that cfDNA in this sample was of poor quality.

DNA Methylation Predicts Drug Response and Clinical Outcome Similar to Gene Expression

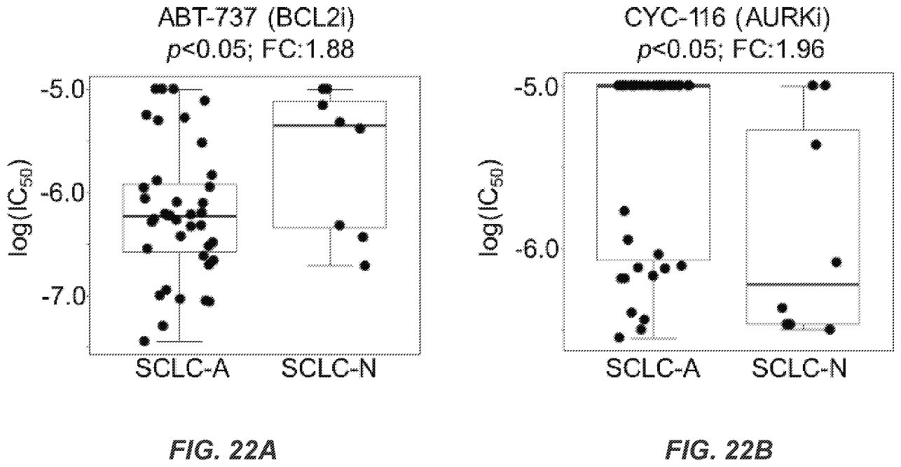

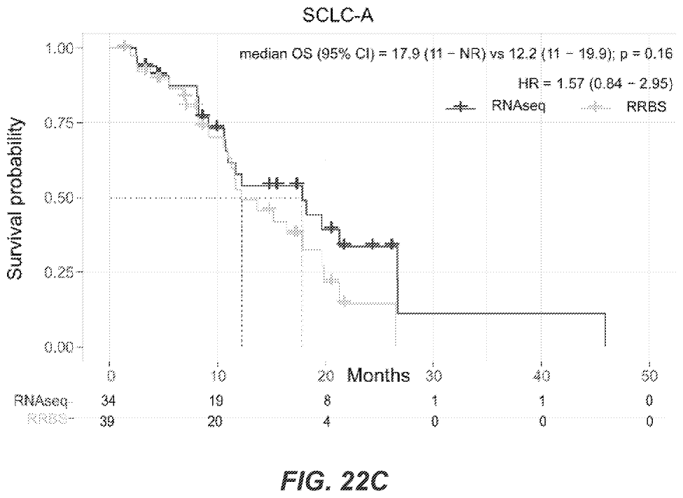

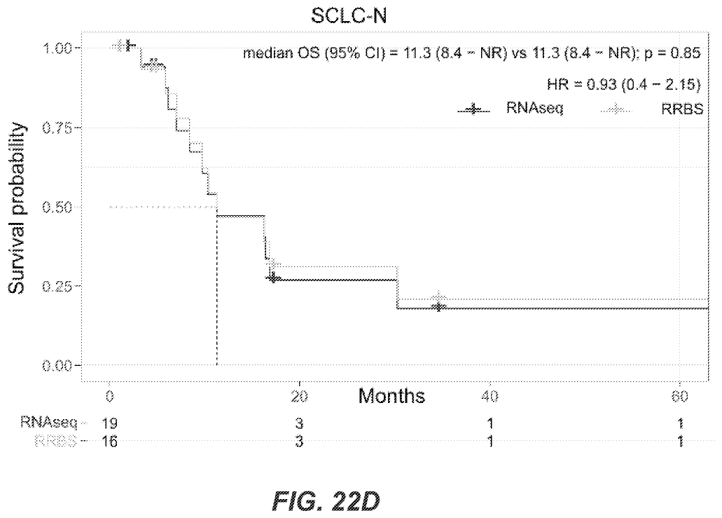

Previously, it was demonstrated that, in vitro, cell lines assigned to SCLC-A and SCLC-N by gene expression possessed unique therapeutic vulnerabilities¹¹. To validate that these same vulnerabilities are preserved using the methylation classifier (FIG. 30), the inventors compared IC50 values for over 400 drugs²⁴ between methylation-assigned SCLC-A and SCLC-N subtypes and identified numerous distinct vulnerabilities between the groups. For example, as demonstrated with the gene expression classifier, SCLC-A cell lines were more sensitive to BCL2 inhibitors (BCL2i) (e.g. ABT-737) (FIG. 22A), while SCLC-N cell lines were more sensitive to Aurora kinase inhibitors (AURKi) (e.g. CYC-116) (FIG. 22B).

Finally, to determine whether methylation- and RNA-based subtyping approaches yielded comparable clinical outcomes among SCLC patients, the inventors used RNAseq gene expression or SCLC-DMC for patients with extensive stage SCLC with known clinical outcomes. While many samples had both RNA and methylation data present, several of the patients were only subtyped by one of the two methods. To ensure adequate statistical power for the analysis, the inventors focused on the two most prevalent subtypes, SCLC-A and SCLC-N, respectively and filtered for ES-SCLC only. Importantly, when comparing the two approaches, overall survival was comparable for patients identified as SCLC-A (HR (95% CI)=1.11 (0.6-2.05); FIG. 22C). Similar results were observed for patients identified as the SCLC-N subtype using the two approaches (HR=1.04 (0.46-2.31); FIG. 22D) when using RNAseq or SCLC-DMC, providing evidence that DNA methylation is able to predict drug response in vitro similar to RNA-based classification and that DNA methylation can indeed be used for clinically reliable subtyping of SCLC.

Methods

Patient Selection

All patients were consented to the GEMINI protocol at the UT MD Anderson Cancer Center (UT MDACC). Totally 105 samples have been selected after pathological examination of the tissue quality. Each sample was required to have >100 tumor cell in each specimen, and at least 2 slides of tissue sections was required for inclusion in the study.

Clinical Data

Clinical data was retrieved from the GEMINI database which included clinical data obtained during treatment at the UT MDACC and consent was provided for accessing the clinical data. Additional data was retrieved manually and reviewed by three board-certified oncologists. For the analysis of survival, overall survival was calculated by time from date of diagnosis to death and patients with lost follow-up were censored at the date where the last information was obtained. Survival analysis was performed using Kaplan-Meier analysis and cox-proportional hazard ratio estimation using the survminer package in R.

Nucleic Acid Extraction

For the nucleic acid extraction, two slides of FFPE tissue samples were cut at 5 μm each. For each sample, tumor area was highlighted by a board-certified Pathologist and macrodissection was used prior to extraction, if necessary. For combined RNA and DNA extraction, the MagMAX FFPE DNA/RNA Ultra Kit (Thermo Fisher Scientific, A31881) was used following the manufacturer's protocol. DNA concentration was assessed using the Qubit 1× dsDNA HS Assay Kit and a Qubit 2.0 fluorimeter. For RNA, concentration was measured using the Qubit RNA high sensitivity (HS) assay kit. RNA quality was analyzed using the Agilent RNA 6000 Pico kit on a 2100 Bioanalyzer.

Following extraction, the expression of the three transcription factors, ASCL1 (BioRad Prime PCR Human Cy5 [qHsaCEP0025578]), NEUROD1 (BioRad PrimePCR Human HEX [qHsaCEP0053288]) and POU2F3 (BioRad PrimePCR Human FAM [qHsaCEP0052042]) was assessed using the Reliance One-Step Multiplex RT-qPCR Supermix (Biorad). Briefly, 2 μl of extracted RNA was directly used for the 1-Step qPCR that includes reverse transcription and amplification in one step on a CFX96 qPCR device (BioRad). GAPDH (BioRad PrimePCR Human Cy5.5 [qHsaCEP0052324]) was used a housekeeper and delta-cT values were calculated for each sample.

For cfDNA extraction, 2-3 ml Plasma obtained in Streck Cell-Free DNA BCT tubes was used for each sample. cfDNA was extracted using the Apostle MiniMax High Efficiency Cell-Free DNA Isolation Kit (Apostle Inc). cfDNA concentration was assessed using the Qubit 1× dsDNA HS Assay Kit and a Qubit 2.0 fluorimeter.

RNAseq

For RNAseq, samples were selected based on the DV200 value and for their expression in qPCR. Upon expert revision, 85 samples have been selected for RNA sequencing. All samples were treated with DNase treatment using DNase I (ThermoFisher, Massachusetts, USA) prior to RNAseq to reduce DNA contamination that might interfere with downstream results. Library generation using the SMARTer Stranded Total RNAseq Kit V3 (Takara Bio USA Inc., California, USA) was performed following the manufacturer's instructions. Final library quantity was measured by KAPA SYBR FAST qPCR and library quality was evaluated using a TapeStation D1000 ScreenTape (Agilent Technologies, CA, USA). Libraries were sequenced on an Illumina NovaSeq instrument (Illumina, California. USA) with a read length configuration of 150 PE for 80M PE reads per sample (40M clusters). Fastq files were quality trimmed using trimmomatic and aligned to the GRCh38 transcriptome using salmon v1.6.0.

Due to the highly degraded RNA and the limited sample input, all RNAseq results were correlated to the qPCR (FIG. 23). Importantly, expression data was highly correlated for ASCL1 (Pearson r=0.94: p<0.0001) and NEUROD1 (Pearson r=0.88: p<0.0001) with good correlation for POU2F3 (Pearson r=0.55: p<0.0001) highlighting the reliability of the RNAseq data despite these limitations. However, NEUROD1 detection using RNAseq was reduced compared to qPCR, highlighting a certain limitation of the RNAseq data in detecting low-expression genes.

RRBS

To analyze DNA Methylation across the genome, RRBS (Reduced Representation Bisulfite Sequencing) was utilized using the Ovation RRBS Methyl-Seq kit (Tecan Group Ltd., Zurich. Switzerland). To account for the highly degraded DNA from FFPE and plasma samples, the material was first treated with one unit of Shrimp Alkaline Phosphatase (New England Biolabs, Ipswich, MA) to remove phosphorylated DNA which might interfere with downstream analysis²³. Briefly, 0.1-100 ng of genomic DNA was digested using MspI, and Illumina-compatible cytosine-methylated adaptor were ligated to the enzyme-digested DNA. For lower concentrations of DNA, adapters were diluted 1:40 to 1:120, in order to decrease the representation of randomly fragmented DNA and adapter-dimers in the final library. RRBS libraries were then visualized using Bioanalyzer High Sensitivity DNA chips (Agilent, Santa Clara. CA), and those passing QC were subsequently sequenced as 100 bp paired-end reads on an Illumina NovaSeq instrument with a target sequencing depth of 300M PE reads (150M clusters). After sequencing, Fastq files were obtained and adapters were trimmed using trimmomatic. Alignment and retrieval of DNA Methylation (in percent of total methylated Cytosines) was performed using Bismark v 0.22 n against the GRCh38 human genome. Samples with <50% mapping rate and, <60M aligned reads were excluded from further analysis. Finally, cytosines with coverage <10 were filtered out to assure high confidence DNA Methylation analysis.

For cell lines and CDX models, 100 ng of RNA was used using the Ovation RRBS Methyl-Seq kit (Tecan Group Ltd., Zurich, Switzerland) as for the clinical samples but without the initial phosphatase step. Sequencing was performed in a single Read 57 bp configuration on a Illumina HiSeq 3000 sequencer. Data processing was performed likewise using Bismark v 0.22. Annotations of methylated regions was performed using the annotatr package and the Hg38 database.

Generation of Predictive Models for Classification Using RNAseq

It was hypothesized that using gene ratios of one gene over another gene might be more robust to classify SCLC across different datasets than using the single expression value. For this purpose, the inventors combined the data retrieved from George et al. comprising of surgical SCLC specimen and the data from the IMPower133 clinical trial as published in Gay C M et al. While for the latter only limited genes were published, the inventors filtered for genes that were present in both datasets that served as training set. ROC analysis was used to define the genes which were mostly associated with one of the four subtypes and selected the top 50 genes for each subtype for further validation which resulted in 181 genes used after removing duplicated genes (Table 26). The inventors then created all different gene ratios of those genes. To select highly relevant gene ratios, the inventors created predictive models, incorporating randomly selected 20 gene ratios per model with 500 distinct models for each of the four subtypes (totally 2000 models created). For the training, the caret package in R was used, and extreme gradient boosting with DART (Dropout Additive Regression Trees) was utilized with repeated cross validation with a 5-fold split and 20 repeats during training. Following, only models with an accuracy=1 on the training data were retained. Those models were then used to define the subtypes in the clinical dataset. In order to obtain the most generalized subtype classification, the inventors used all models for the prediction and if >50% of the models agreed on the subtype, the subtype was called based on this consensus classification. Samples with less than 50% agreement are called “equivocal” as a clear classification could not be obtained with the current methodology.

TABLE 26

| | | | |
|---|---|---|---|
| SH2D2A | WNT11 | POU2F3 | LCP2 |
| SCN2A | NHLH1 | RIMKLA | CTSS |
| SCN3A | SSTR2 | KCNF1 | CD53 |
| SCG2 | ID2 | ELAVL4 | PTPN22 |
| DDC | ARHGEF38 | TCERG1L | GIMAP2 |
| ICA1 | SHF | CPLX2 | CD74 |
| CNKSR3 | SEMA6A | CHGA | FGL2 |
| ASCL1 | NEUROD1 | KCNH6 | CXCR6 |
| FOXA2 | TEC | FAM105A | TIGIT |
| PTPRN2 | FNDC5 | ANO7 | ITK |
| FZD7 | RAB11FIP1 | FBLN7 | SLFN12L |
| PREX1 | SLC17A6 | KCNK3 | SASH3 |
| SYT1 | SH2D3A | ST8SIA5 | RASAL3 |
| ZNF217 | POF1B | GRIK3 | HLA-DMA |
| LDLRAP1 | PRIMA1 | C1QL1 | HLA-DMB |
| MGAT4C | TFF3 | CHD7 | ARHGAP30 |
| CXXC4 | EFNB1 | KCNMB2 | CASP1 |
| RILPL2 | ALDH1A1 | RTBDN | SELPLG |
| RGL1 | CASP4 | HCN2 | IL10RA |
| PPARD | HUNK | RGS7 | PTAFR |
| ETS2 | SMO | MAP2 | CCR5 |
| GJB1 | RBFOX3 | SOX9 | P2RY10 |
| HABP2 | ELF1 | TRIM9 | CASP10 |
| RNF148 | ALK | FAM131A | EVI2A |
| STX19 | HSH2D | MYC | ITGAL |
| SEC11C | PRKX | NRXN1 | TNFAIP8L2 |
| CADPS2 | BCL3 | RFX2 | DOK2 |
| EMP3 | GLI2 | UNC80 | PTPN7 |
| FAM124A | CHRNA3 | INSM1 | CSF2RB |
| SYT4 | CEACAM5 | GRP | ICOS |
| RARG | MDGA1 | VWA5B2 | WIPF1 |
| LRMP | ATOH7 | ISL1 | CCR2 |
| NCALD | MMP24 | ANXA4 | CYBB |
| PRUNE2 | VAV1 | IL2RB | SLAMF1 |
| REST | KLF4 | GIMAP7 | |
| FAM78A | PCDH8 | KLHL6 | |
| RNF133 | SEZ6L | PTPRC | |
| RGS17 | CPE | NLRC3 | |
| MAT2B | PCSK2 | CCDC69 | |
| NELL1 | CA8 | MPEG1 | |
| BLNK | C11orf53 | SNX20 | |
| BTN2A2 | PCSK1 | HLA-DRA | |
| GCH1 | CAMK2N2 | JAK3 | |
| EFNA4 | JAKMIP2 | ARHGAP15 | |
| LCN15 | GNAO1 | TNFRSF1B | |
| SMPD3 | DGKB | GIMAP4 | |
| CPVL | GFI1B | CD2 | |
| EXTL3 | ASCL2 | IL2RG | |
| ADCY7 | CACNA1A | CD96 | |

Generation of Predictive Models for Classification Using DNA Methylation Data

To generate models with broader applicability, the inventors combined data from the cell lines and the clinical GEMINI cohort in order to tune models to work across different sample types. The selected DNA Methylation sites were filtered to be present in both datasets. Following, the inventors performed ROC analysis on the combined set to select the methylation sites that had the highest association with one of the four subtypes. The inventors selected the top 500 methylation sites (by AUROC, Tables 27-30) for each of the four subtypes and created models by randomly selecting 5, 10, or 20 methylation per subtype per model. For each number of methylation sites, 500 models were created using xgboost with DART and 3-fold cross validation with 20 repeats using the training set. Due to the presence of missing values in the dataset, the inventors used median imputation to replace missing values. Models were used to predict the subtype of the samples in the training and testing set. Only models with an accuracy=1 were maintained and used to predict the subtype on the full GEMINI dataset. Similar to the RNAseq approach, a subtype was called when >50% agreed on the subtype. If <50% agreement was achieved, the subtype was classified as “equivocal” due to the lack of consensus. The same models and classification strategy were pursued for the cfDNA samples as well as for cell lines.

TABLE 27
DNA methylation sites associated with SCLC-A

| MethylationSite | AUC | threshold | specificity | sensitivity |
|---|---|---|---|---|
| chr17: 74961036 | 1 | 82.9326923 | 1 | 1 |
| chr17: 74961013 | 0.978788 | 53.5294118 | 0.90909091 | 1 |
| chr18: 59062159 | 0.965686 | 92.9802956 | 1 | 0.85294118 |
| chr19: 13506705 | 0.964286 | 75.5007257 | 1 | 0.91428571 |
| chr9: 134815629 | 0.963265 | 77.7985075 | 0.85714286 | 0.97142857 |
| chr21: 41180000 | 0.961616 | 91.1538462 | 0.86666667 | 0.93939394 |
| chr9: 93014411 | 0.959034 | 64.9122807 | 0.92857143 | 0.94117647 |
| chr9: 114211349 | 0.958824 | 76.1111111 | 1 | 0.85294118 |
| chr9: 134810271 | 0.955236 | 82.9710145 | 0.875 | 0.94594595 |
| chr19: 3385734 | 0.952941 | 80.7407407 | 1 | 0.82352941 |
| chr6: 51213948 | 0.952679 | 10.5555556 | 0.875 | 0.91428571 |
| chr18: 27786347 | 0.94958 | 7.76723277 | 1 | 0.79411765 |
| chr6: 168638645 | 0.94958 | 94.4837162 | 1 | 0.79411765 |
| chr9: 134815657 | 0.94898 | 85.6516291 | 0.92857143 | 0.91428571 |
| chr9: 134815611 | 0.948962 | 48.3333333 | 0.94117647 | 0.85294118 |
| chr16: 84519934 | 0.948529 | 38.9569161 | 0.9375 | 0.85294118 |
| chr20: 20364625 | 0.948148 | 1.54838456 | 0.93333333 | 0.88888889 |
| chr5: 172103442 | 0.947619 | 81.1764706 | 1 | 0.8 |
| chr20: 22598951 | 0.947059 | 90.8333333 | 0.8 | 0.94117647 |
| chr16: 85355101 | 0.946691 | 86.0226105 | 0.9375 | 0.88235294 |
| chr9: 134815628 | 0.946367 | 52.2435897 | 1 | 0.85294118 |
| chr20: 20364629 | 0.944762 | 7.53052917 | 0.93333333 | 0.91428571 |
| chr19: 511206 | 0.944444 | 27.1929825 | 0.83333333 | 1 |
| chr6: 168149181 | 0.944328 | 72.4164134 | 0.85714286 | 0.94117647 |
| chr7: 100122306 | 0.943137 | 18.3333333 | 0.93333333 | 0.85294118 |
| chr16: 84519930 | 0.943015 | 73.3333333 | 0.875 | 0.91176471 |
| chr8: 58989473 | 0.94246 | 92.2619048 | 0.85714286 | 0.91666667 |
| chr18: 27786294 | 0.941176 | 14.5511785 | 0.85714286 | 0.91176471 |
| chr20: 22557408 | 0.940541 | 60.2564103 | 0.8 | 1 |
| chr9: 128177708 | 0.940045 | 80.7608696 | 0.92307692 | 0.85294118 |
| chr7: 157633760 | 0.939394 | 68.3333333 | 0.92307692 | 0.96969697 |
| chr6: 40349351 | 0.9375 | 21.5384615 | 0.8125 | 0.97142857 |
| chr6: 157229362 | 0.937255 | 5.90277778 | 0.93333333 | 0.91176471 |
| chr13: 84927994 | 0.937229 | 79.3800539 | 1 | 0.81818182 |
| chr4: 8101279 | 0.935556 | 28.0359435 | 0.8 | 1 |
| chr22: 41667328 | 0.934743 | 15.8874459 | 1 | 0.82352941 |
| chr5: 176843804 | 0.931985 | 64.6428571 | 0.9375 | 0.76470588 |
| chr5: 176529007 | 0.931373 | 62.8787879 | 0.93333333 | 0.88235294 |
| chr4: 137426451 | 0.930804 | 94.567355 | 1 | 0.84375 |
| chr6: 40349318 | 0.930796 | 80.5405405 | 0.94117647 | 0.82352941 |
| chr11: 11578650 | 0.930303 | 29.1509084 | 0.86666667 | 0.87878788 |
| chr20: 20364628 | 0.92963 | 2.43313796 | 0.93333333 | 0.83333333 |
| chr6: 40349319 | 0.929464 | 71.4884021 | 0.875 | 0.91428571 |
| chr17: 48190199 | 0.929412 | 83.9893617 | 0.86666667 | 0.91176471 |
| chr7: 100122301 | 0.929412 | 18.7575758 | 0.93333333 | 0.82352941 |
| chr1: 226589216 | 0.929333 | 72.0779221 | 0.8 | 0.92 |
| chr22: 40022901 | 0.929167 | 89.4444444 | 0.93333333 | 0.84375 |
| chr6: 37078047 | 0.929167 | 82.8008361 | 0.93333333 | 0.84375 |
| chr9: 134765725 | 0.927619 | 64.4957983 | 0.86666667 | 0.82857143 |
| chr20: 20364641 | 0.926667 | 11.673699 | 0.8 | 0.94285714 |
| chr18: 77020770 | 0.926573 | 84.4129555 | 0.92307692 | 0.84848485 |
| chr17: 3695881 | 0.926563 | 13.3928571 | 0.9 | 0.90625 |
| chr19: 38253123 | 0.92562 | 68.115942 | 0.90909091 | 0.86363636 |
| chr9: 114255802 | 0.92542 | 83.952381 | 0.92857143 | 0.85294118 |
| chr7: 157017749 | 0.925408 | 21.1929825 | 0.77272727 | 0.97435897 |
| chr17: 74823356 | 0.925325 | 91.6666667 | 0.95238095 | 0.77272727 |
| chr9: 117355642 | 0.92435 | 78.8353863 | 0.88888889 | 0.85106383 |
| chr6: 51213976 | 0.924107 | 36.6666667 | 1 | 0.8 |
| chr7: 151045548 | 0.923197 | 35.1470588 | 0.81818182 | 0.93103448 |
| chr16: 4537495 | 0.923077 | 31.423959 | 0.875 | 0.8974359 |
| chr2: 45274755 | 0.923021 | 15.2413793 | 0.77777778 | 0.94117647 |
| chr8: 139674518 | 0.922857 | 65.3966132 | 0.8 | 0.94285714 |
| chr12: 132134577 | 0.922727 | 6.2745098 | 0.90909091 | 0.86666667 |
| chr9: 134885740 | 0.922549 | 33.7912088 | 0.93333333 | 0.82352941 |
| chr16: 4537572 | 0.922323 | 18.5714286 | 0.82352941 | 0.94871795 |
| chr17: 1961091 | 0.922269 | 15.0987654 | 0.92857143 | 0.88235294 |
| chr6: 51213977 | 0.921569 | 22.5 | 0.93333333 | 0.79411765 |
| chr11: 888785 | 0.921512 | 95.7427536 | 0.95 | 0.76744186 |
| chr9: 134815626 | 0.92128 | 48.3050847 | 0.88235294 | 0.88235294 |
| chr13: 110011941 | 0.920507 | 84.6978558 | 0.85714286 | 0.90322581 |
| chr19: 13372294 | 0.920202 | 99.1803279 | 1 | 0.75757576 |
| chr16: 84519955 | 0.920037 | 38.3390706 | 1 | 0.73529412 |
| chr20: 20364626 | 0.92 | 6.9047619 | 0.86666667 | 0.88571429 |
| chr20: 20364640 | 0.919444 | 5.15898767 | 0.93333333 | 0.83333333 |
| chr17: 57874806 | 0.919255 | 86.2997658 | 0.91304348 | 0.82857143 |
| chr6: 168638634 | 0.919118 | 91.4285714 | 1 | 0.76470588 |
| chr7: 14372215 | 0.919118 | 55.8333333 | 0.78571429 | 0.97058824 |
| chr22: 50010360 | 0.918919 | 81.5340909 | 0.88888889 | 0.86486486 |
| chr14: 56814573 | 0.918627 | 78.8888889 | 0.8 | 0.88235294 |
| chr13: 112641259 | 0.918168 | 84.3171296 | 0.83333333 | 0.89189189 |
| chr7: 146633722 | 0.917708 | 90.8902692 | 0.86666667 | 0.875 |
| chr7: 157633743 | 0.917647 | 73.5431235 | 0.86666667 | 0.97058824 |
| chr21: 43758530 | 0.917647 | 60.2941176 | 0.93333333 | 0.82352941 |
| chr6: 168538915 | 0.917647 | 68.0489102 | 0.93333333 | 0.73529412 |
| chr5: 176843791 | 0.917279 | 53.9130435 | 0.75 | 0.97058824 |
| chr2: 46983311 | 0.917094 | 89.039039 | 0.88461538 | 0.88888889 |
| chr2: 239300749 | 0.91619 | 89.6969697 | 0.93333333 | 0.8 |
| chr7: 157998148 | 0.915966 | 83.974359 | 1 | 0.73529412 |
| chr6: 34128215 | 0.915686 | 94.0972222 | 0.93333333 | 0.82352941 |
| chr13: 112106712 | 0.915584 | 62.8101402 | 0.85714286 | 0.84848485 |
| chr16: 3999700 | 0.915179 | 70.5627706 | 0.82142857 | 0.89583333 |
| chr9: 114172063 | 0.915179 | 51.8154762 | 1 | 0.75 |
| chr9: 134792142 | 0.915179 | 45.1548452 | 0.85714286 | 0.90625 |
| chr11: 11578688 | 0.915152 | 74.7364458 | 0.86666667 | 0.87878788 |
| chr7: 157833048 | 0.914815 | 71.1708566 | 0.8 | 1 |
| chr1: 151346323 | 0.914643 | 56.9385593 | 0.78571429 | 0.92 |
| chr21: 38661697 | 0.914469 | 55.2811245 | 0.93103448 | 0.80392157 |
| chr1: 226588954 | 0.91413 | 73.8636364 | 0.86956522 | 0.875 |
| chr20: 6232329 | 0.914063 | 85.4166667 | 0.9 | 0.78125 |
| chr9: 134792130 | 0.914063 | 12.4620061 | 1 | 0.71875 |
| chr9: 134785070 | 0.91342 | 57.4786325 | 0.78571429 | 0.90909091 |
| chr9: 134815627 | 0.913265 | 33.7108014 | 1 | 0.71428571 |
| chr9: 134592825 | 0.913131 | 16.025641 | 1 | 0.78787879 |
| chr16: 84519980 | 0.912879 | 42.0168067 | 0.9375 | 0.84848485 |
| chr7: 100122290 | 0.912745 | 29.1666667 | 0.93333333 | 0.85294118 |
| chr11: 11973687 | 0.912587 | 83.974359 | 0.92307692 | 0.78787879 |
| chr19: 38253193 | 0.912482 | 71.9362219 | 1 | 0.70731707 |
| chr14: 44405368 | 0.912338 | 78.030303 | 0.92857143 | 0.81818182 |
| chr16: 4260161 | 0.91226 | 80.3846154 | 0.92307692 | 0.875 |
| chr2: 28411073 | 0.912121 | 90.9459459 | 0.86666667 | 0.84848485 |
| chr1: 50214861 | 0.912103 | 72.6370045 | 0.82758621 | 0.82352941 |
| chr16: 84519943 | 0.911932 | 42.481203 | 0.9375 | 0.84848485 |
| chr22: 19933408 | 0.911828 | 76.0714286 | 0.86666667 | 0.90322581 |
| chr11: 68833022 | 0.911765 | 82.8431373 | 0.93333333 | 0.73529412 |
| chr7: 73985377 | 0.911765 | 56.684492 | 1 | 0.82352941 |
| chr9: 93014375 | 0.911765 | 63.3971292 | 0.78571429 | 0.91176471 |
| chr9: 114183920 | 0.911765 | 43.9613527 | 1 | 0.73529412 |
| chr16: 4260632 | 0.911585 | 8.71212121 | 0.95 | 0.75609756 |
| chr9: 133558991 | 0.910985 | 72.2128378 | 0.91666667 | 0.88636364 |
| chr6: 168638701 | 0.910784 | 64.3483023 | 0.86666667 | 0.88235294 |
| chr7: 157658598 | 0.910784 | 82.2727273 | 0.73333333 | 0.97058824 |
| chr19: 13506729 | 0.910204 | 88.1944444 | 0.92857143 | 0.85714286 |
| chr19: 13483181 | 0.910101 | 94.4698703 | 0.86666667 | 0.84848485 |
| chr16: 84519942 | 0.909926 | 72.7018482 | 0.875 | 0.88235294 |
| chr11: 76784947 | 0.909903 | 57.5268817 | 0.82142857 | 0.88636364 |
| chr11: 65551424 | 0.909804 | 25.9109312 | 0.86666667 | 0.82352941 |
| chr7: 2193699 | 0.909804 | 79.5138889 | 0.8 | 0.91176471 |
| chr5: 179258981 | 0.909677 | 45.1923077 | 1 | 0.67741935 |
| chr14: 99518884 | 0.909664 | 52.1492921 | 0.71428571 | 0.97058824 |
| chr18: 27786301 | 0.909664 | 8.16056191 | 0.92857143 | 0.85294118 |
| chr11: 64056580 | 0.909659 | 88.4277466 | 0.8 | 0.86363636 |
| chr6: 169251928 | 0.909399 | 72.3011364 | 0.83333333 | 0.93023256 |
| chr9: 134486435 | 0.909314 | 93.0952381 | 0.75 | 0.94117647 |
| chr12: 132134595 | 0.909091 | 11.6161616 | 0.9375 | 0.81818182 |
| chr16: 84519935 | 0.909091 | 64.1025641 | 0.8125 | 0.93939394 |
| chr21: 41179996 | 0.909091 | 83.1300813 | 0.8 | 0.90909091 |
| chr14: 105392911 | 0.908929 | 93.9338235 | 0.9 | 0.89285714 |
| chr7: 40330569 | 0.908824 | 16.7582298 | 0.86666667 | 0.85294118 |
| chr13: 111622152 | 0.908669 | 88.5620915 | 1 | 0.81578947 |
| chr2: 98823202 | 0.908547 | 94.6778711 | 1 | 0.69230769 |
| chr17: 29972557 | 0.908179 | 43.1354515 | 1 | 0.77777778 |
| chr9: 134815918 | 0.908163 | 54.4230769 | 1 | 0.77142857 |
| chr1: 213905614 | 0.908152 | 81.8910256 | 0.91304348 | 0.825 |
| chr7: 2607131 | 0.908027 | 91.2878788 | 0.84615385 | 0.84782609 |
| chr4: 8101234 | 0.907867 | 85.7843137 | 0.85714286 | 0.82608696 |
| chr12: 103012503 | 0.907778 | 41.2235009 | 0.81481481 | 0.84 |
| chr14: 44262090 | 0.907563 | 88.7778412 | 1 | 0.76470588 |
| chr6: 82363058 | 0.907482 | 32.3643411 | 0.91304348 | 0.86046512 |
| chr9: 114170339 | 0.907121 | 46.547619 | 0.94117647 | 0.78947368 |
| chr6: 168138608 | 0.907051 | 89.1812865 | 1 | 0.71794872 |
| chr12: 11856447 | 0.906926 | 44.8538012 | 0.92857143 | 0.81818182 |
| chr19: 13634667 | 0.906863 | 90.6926407 | 0.86666667 | 0.85294118 |
| chr9: 93722421 | 0.906863 | 91.6083916 | 0.93333333 | 0.76470588 |
| chr9: 114172123 | 0.906863 | 35.7336957 | 1 | 0.73529412 |
| chr17: 29565848 | 0.906746 | 18.2623049 | 0.85714286 | 0.94444444 |
| chr11: 64353390 | 0.906636 | 10.7526882 | 1 | 0.72222222 |
| chr9: 90908530 | 0.906452 | 83.974359 | 1 | 0.70967742 |
| chr16: 3999757 | 0.906071 | 80.7539683 | 1 | 0.74 |
| chr11: 65551425 | 0.905882 | 16.5906128 | 1 | 0.70588235 |
| chr9: 114306707 | 0.905754 | 16.4351852 | 1 | 0.72222222 |
| chr19: 2354863 | 0.905556 | 91.9047619 | 0.93333333 | 0.80555556 |
| chr9: 136289024 | 0.905405 | 19.7374897 | 0.8125 | 0.91891892 |
| chr6: 107633714 | 0.905051 | 90.4157858 | 0.86666667 | 0.93939394 |
| chr7: 157017791 | 0.905012 | 12.2360248 | 0.86363636 | 0.92307692 |
| chr6: 168496984 | 0.904977 | 81.5104167 | 0.84615385 | 0.91176471 |
| chr3: 45819148 | 0.904902 | 93.4443657 | 0.93333333 | 0.73529412 |
| chr18: 77102188 | 0.904429 | 72.5694444 | 0.76923077 | 0.93939394 |
| chr12: 105784446 | 0.904327 | 65.04329 | 0.75862069 | 0.92156863 |
| chr17: 29972550 | 0.904321 | 60.4347826 | 0.83333333 | 0.86111111 |
| chr11: 119702010 | 0.903922 | 41.0441044 | 0.8 | 0.85294118 |
| chr5: 1316124 | 0.903922 | 0.28409091 | 0.93333333 | 0.76470588 |
| chr7: 73966925 | 0.90368 | 70.5300128 | 0.78571429 | 0.90909091 |
| chr3: 53702765 | 0.90368 | 94.7222222 | 0.92857143 | 0.81818182 |
| chr11: 118924054 | 0.903646 | 52.2727273 | 0.91666667 | 0.90625 |
| chr19: 51077543 | 0.903333 | 56.4705882 | 0.8 | 0.93333333 |
| chr17: 81246338 | 0.902902 | 75.5555556 | 0.92857143 | 0.8125 |
| chr12: 3077928 | 0.902872 | 22.5396825 | 0.96296296 | 0.75510204 |
| chr12: 130460997 | 0.902748 | 56.25 | 0.81818182 | 0.95348837 |
| chr7: 157963798 | 0.902715 | 88.1818182 | 0.92307692 | 0.79411765 |
| chr12: 130483815 | 0.902597 | 90.6445993 | 0.85714286 | 0.87878788 |
| chr7: 157963771 | 0.902344 | 88.3116883 | 0.91666667 | 0.875 |
| chr14: 64942593 | 0.901961 | 52.7525711 | 0.86666667 | 0.88235294 |
| chr18: 59203056 | 0.901961 | 92.5824176 | 0.86666667 | 0.91176471 |
| chr19: 13463517 | 0.901961 | 82.8571429 | 0.86666667 | 0.88235294 |
| chr20: 20364646 | 0.901852 | 18.079096 | 0.73333333 | 1 |
| chr7: 157987469 | 0.901852 | 88.75 | 0.8 | 0.94444444 |
| chr2: 216380208 | 0.901786 | 43.75 | 0.8125 | 0.91428571 |
| chr20: 16806905 | 0.901681 | 90.8033827 | 0.82352941 | 0.8 |
| chr17: 74961041 | 0.901515 | 73.2142857 | 0.81818182 | 1 |
| chr3: 176190636 | 0.901182 | 91.2878788 | 0.75 | 0.94594595 |
| chr20: 22652824 | 0.901087 | 80.1960784 | 0.95652174 | 0.8 |
| chr6: 37078083 | 0.901075 | 72.4747475 | 1 | 0.67741935 |
| chr14: 44405319 | 0.901042 | 63.0681818 | 0.8 | 0.9375 |
| chr18: 72677631 | 0.901042 | 92.1212121 | 0.875 | 0.86111111 |
| chr9: 136686592 | 0.90101 | 20.5263158 | 0.93333333 | 0.78787879 |
| chr2: 216986073 | 0.900735 | 15.0565262 | 0.8125 | 0.91176471 |
| chr2: 10040203 | 0.900538 | 63.8181818 | 0.83333333 | 0.87096774 |
| chr21: 38636406 | 0.900483 | 83.1140351 | 1 | 0.64444444 |
| chr9: 134824994 | 0.900433 | 79.2857143 | 0.92857143 | 0.78787879 |
| chr12: 119158214 | 0.900426 | 73.7068966 | 0.8 | 0.89361702 |
| chr7: 5795946 | 0.900293 | 15.7407407 | 0.90909091 | 0.80645161 |
| chr22: 19933350 | 0.90021 | 42.1296296 | 0.92857143 | 0.82352941 |
| chr9: 134785048 | 0.90021 | 84.1085271 | 1 | 0.79411765 |
| chr13: 111559772 | 0.9 | 85.9895484 | 0.92857143 | 0.83333333 |
| chr16: 88640994 | 0.9 | 65.6862745 | 0.8 | 0.90909091 |
| chr17: 29972549 | 0.9 | 62.7815315 | 0.86666667 | 0.85714286 |
| chr6: 168130574 | 0.9 | 85.9342302 | 0.86666667 | 0.82352941 |
| chr6: 168130602 | 0.9 | 98.4115884 | 0.93333333 | 0.73529412 |
| chr9: 114183394 | 0.9 | 52.6129032 | 0.78571429 | 0.88 |
| chr9: 120326115 | 0.9 | 91.2878788 | 0.85 | 0.87804878 |
| chr3: 177516507 | 0.899909 | 83.4094803 | 0.75 | 0.91304348 |
| chr7: 110084403 | 0.899767 | 77.3504274 | 0.84615385 | 0.87878788 |
| chr22: 50010361 | 0.899729 | 72.0486111 | 0.77777778 | 0.87804878 |
| chr18: 76681573 | 0.899671 | 91.9871795 | 0.9375 | 0.76315789 |
| chr7: 158485004 | 0.89946 | 91.0199005 | 0.78947368 | 0.94871795 |
| chr21: 38661630 | 0.899052 | 48.3333333 | 0.78571429 | 0.85714286 |
| chr15: 53659351 | 0.899038 | 6.51260504 | 0.84615385 | 0.90625 |
| chr19: 13347369 | 0.89902 | 80.8035714 | 0.73333333 | 0.97058824 |
| chr19: 39163809 | 0.89902 | 84.2588446 | 0.93333333 | 0.91176471 |
| chr17: 48620272 | 0.89899 | 60.4264292 | 0.8 | 0.90909091 |
| chr18: 71652249 | 0.89899 | 38.1048387 | 0.86666667 | 0.90909091 |
| chr6: 168667391 | 0.89899 | 91.9871795 | 0.86666667 | 0.81818182 |
| chr10: 133309889 | 0.898966 | 14.7186147 | 0.79310345 | 0.88 |
| chr1: 241210311 | 0.898864 | 81.4381271 | 0.8 | 0.93181818 |
| chr19: 46770637 | 0.89881 | 13.8095238 | 0.82142857 | 0.8627451 |
| chr20: 22556743 | 0.898649 | 66.1714771 | 0.8 | 0.94594595 |
| chr2: 47227055 | 0.898571 | 20.8695652 | 0.78571429 | 0.92 |
| chr7: 150974698 | 0.898485 | 17.1568627 | 0.81818182 | 0.9 |
| chr20: 22653042 | 0.898438 | 56.547619 | 0.79166667 | 0.925 |
| chr14: 73228468 | 0.898311 | 40.9803922 | 0.75862069 | 0.91836735 |
| chr19: 18422970 | 0.898295 | 89.2045455 | 0.86363636 | 0.8 |
| chr21: 40144935 | 0.898095 | 88.6752137 | 0.8 | 0.94285714 |
| chr16: 85355024 | 0.898039 | 70.8333333 | 0.8 | 0.94117647 |
| chr19: 13575256 | 0.898039 | 11.0357242 | 0.86666667 | 0.91176471 |
| chr9: 134690692 | 0.897917 | 57.7380952 | 1 | 0.75 |
| chr6: 82363052 | 0.897877 | 41.7774086 | 0.91304348 | 0.8372093 |
| chr7: 122307220 | 0.897771 | 69.0981432 | 0.95833333 | 0.74418605 |
| chr7: 157633801 | 0.897436 | 60.7669617 | 0.84615385 | 1 |
| chr1: 43439683 | 0.897059 | 80.9428785 | 0.86666667 | 0.91176471 |
| chr16: 64387 | 0.897059 | 10.6442577 | 1 | 0.64705882 |
| chr2: 216986079 | 0.897059 | 13.1642512 | 0.875 | 0.85294118 |
| chr9: 134885778 | 0.897059 | 15.4061625 | 1 | 0.73529412 |
| chr4: 128846433 | 0.89697 | 17.9006731 | 0.93333333 | 0.6969697 |
| chr1: 43550363 | 0.896552 | 3.92307692 | 1 | 0.65517241 |
| chr3: 9180988 | 0.896552 | 75.3571429 | 0.86206897 | 0.83333333 |
| chr19: 511219 | 0.896465 | 17.1428571 | 0.83333333 | 0.96969697 |
| chr20: 62711715 | 0.896104 | 36.9318182 | 0.90909091 | 0.96428571 |
| chr11: 47327260 | 0.896078 | 85.1648352 | 0.8 | 0.97058824 |
| chr6: 51214001 | 0.896078 | 15.7960199 | 0.8 | 0.88235294 |
| chr5: 10652237 | 0.89596 | 92.5824176 | 0.93333333 | 0.75757576 |
| chr4: 6944534 | 0.895946 | 83.4841629 | 0.95 | 0.81081081 |
| chr21: 38599607 | 0.895833 | 84.5238095 | 0.86666667 | 0.875 |
| chr6: 168440545 | 0.895833 | 72.6775956 | 1 | 0.69444444 |
| chr1: 18794157 | 0.895833 | 13.8095238 | 0.91666667 | 0.78125 |
| chr8: 52150618 | 0.895433 | 53.2352941 | 0.84615385 | 0.84375 |
| chr9: 133662377 | 0.895433 | 86.8055556 | 0.84615385 | 0.84375 |
| chr16: 4537619 | 0.895349 | 35.8974359 | 0.76470588 | 0.93023256 |
| chr19: 39307494 | 0.895221 | 14.7157191 | 0.8125 | 0.94117647 |
| chr19: 55254005 | 0.895098 | 49.7917751 | 0.86666667 | 0.85294118 |
| chr12: 130535047 | 0.895098 | 94.5588235 | 0.8 | 0.94117647 |
| chr5: 82804 | 0.895098 | 89.8989899 | 1 | 0.70588235 |
| chr6: 34128233 | 0.895098 | 99.6453901 | 0.93333333 | 0.76470588 |
| chr12: 11856451 | 0.895022 | 61.2979506 | 0.85714286 | 0.84848485 |
| chr19: 13506452 | 0.894792 | 41.7867147 | 0.8 | 0.9375 |
| chr17: 77452817 | 0.894737 | 87.0833333 | 0.94736842 | 0.77777778 |
| chr6: 109290309 | 0.894643 | 83.5888975 | 0.8125 | 0.91428571 |
| chr21: 38661664 | 0.894523 | 59.2451144 | 0.96551724 | 0.80392157 |
| chr10: 133309857 | 0.894483 | 7.91637303 | 0.89655172 | 0.78 |
| chr21: 34667592 | 0.894271 | 71.4052288 | 0.85 | 0.83333333 |
| chr7: 100477920 | 0.894231 | 87.8676471 | 0.84615385 | 0.8125 |
| chr20: 38294975 | 0.894118 | 84.3939394 | 0.8 | 0.94117647 |
| chr21: 41648785 | 0.894118 | 81.9642857 | 0.8 | 0.85294118 |
| chr21: 43758525 | 0.894118 | 71.657754 | 0.93333333 | 0.79411765 |
| chr9: 134822835 | 0.894044 | 14.5104895 | 0.94736842 | 0.76315789 |
| chr16: 3728390 | 0.893599 | 99.2537313 | 0.82352941 | 0.91176471 |
| chr16: 29604945 | 0.893571 | 73.2924869 | 0.89285714 | 0.82 |
| chr1: 41727346 | 0.893534 | 84.1995842 | 0.91304348 | 0.69230769 |
| chr21: 45032479 | 0.893382 | 45.2586207 | 0.75 | 0.94117647 |
| chr1: 39954061 | 0.893333 | 95.9166667 | 1 | 0.7 |
| chr17: 29972554 | 0.893333 | 39.8026316 | 1 | 0.71428571 |
| chr6: 151809197 | 0.893333 | 43.1272727 | 0.86666667 | 0.82857143 |
| chr12: 111287675 | 0.893189 | 38.962766 | 0.76470588 | 0.86842105 |
| chr19: 13463510 | 0.893137 | 82.0855615 | 0.93333333 | 0.70588235 |
| chr8: 52150574 | 0.893029 | 71.6402116 | 0.69230769 | 0.96875 |
| chr19: 13014442 | 0.892929 | 55.1547988 | 0.66666667 | 1 |
| chr4: 8164496 | 0.892929 | 84.3076923 | 1 | 0.66666667 |
| chr7: 8417325 | 0.892929 | 90.7670455 | 0.93333333 | 0.75757576 |
| chr7: 157673872 | 0.892857 | 70.8333333 | 0.78571429 | 0.90909091 |
| chr2: 107461969 | 0.892857 | 85.1648352 | 0.92857143 | 0.71875 |
| chr9: 36781051 | 0.892857 | 88.1944444 | 0.91666667 | 0.80952381 |
| chr4: 138912435 | 0.892593 | 12.9935721 | 0.8 | 0.94444444 |
| chr7: 157594362 | 0.892593 | 94.2222222 | 0.86666667 | 0.83333333 |
| chr2: 237733847 | 0.892424 | 83.6666667 | 0.81818182 | 0.86666667 |
| chr20: 22652808 | 0.892391 | 34.2032967 | 0.82608696 | 0.9 |
| chr14: 64942648 | 0.892157 | 65.6698565 | 0.8 | 0.88235294 |
| chr16: 4260588 | 0.892073 | 15.4019926 | 0.95 | 0.7804878 |
| chr7: 157603987 | 0.891964 | 73.3766234 | 0.6875 | 0.97142857 |
| chr7: 157540317 | 0.891892 | 93.2126697 | 0.86666667 | 0.78378378 |
| chr13: 111515678 | 0.891882 | 80.2269571 | 0.93103448 | 0.72916667 |
| chr17: 67488327 | 0.891775 | 72.0688869 | 0.92857143 | 0.78787879 |
| chr7: 73990185 | 0.891775 | 66.5994624 | 0.92857143 | 0.78787879 |
| chr10: 43849791 | 0.891705 | 29.6703297 | 0.92857143 | 0.77419355 |
| chr17: 1961149 | 0.891667 | 27.7140335 | 0.86666667 | 0.875 |
| chr6: 82585382 | 0.891176 | 56.6964286 | 0.86666667 | 0.85294118 |
| chr1: 224184849 | 0.891176 | 92.0254058 | 0.86666667 | 0.79411765 |
| chr2: 182053988 | 0.891141 | 89.1812865 | 0.94444444 | 0.75675676 |
| chr21: 41508507 | 0.890977 | 42.2619048 | 0.94736842 | 0.73809524 |
| chr15: 85942771 | 0.890909 | 90.4545455 | 0.8 | 0.90909091 |
| chr10: 78139388 | 0.890741 | 85.3571429 | 0.95 | 0.66666667 |
| chr9: 126420967 | 0.890704 | 5.84381551 | 0.92592593 | 0.76470588 |
| chr15: 67806290 | 0.890625 | 82.8431373 | 0.8 | 0.84375 |
| chr7: 157963797 | 0.890625 | 78.4161491 | 0.83333333 | 0.9375 |
| chr9: 113593968 | 0.890625 | 73.8636364 | 1 | 0.71875 |
| chr7: 157875450 | 0.890196 | 78.3625731 | 0.8 | 0.94117647 |
| chr7: 157875495 | 0.890196 | 62.6409018 | 0.86666667 | 0.85294118 |
| chr14: 64942637 | 0.890196 | 19.6275946 | 0.86666667 | 0.79411765 |
| chr19: 55254025 | 0.890196 | 47.2463768 | 0.93333333 | 0.82352941 |
| chr1: 205447752 | 0.890152 | 56.4144737 | 0.83333333 | 0.84848485 |
| chr16: 84519956 | 0.890152 | 56.9444444 | 0.875 | 0.81818182 |
| chr9: 134797123 | 0.889889 | 40.6896552 | 1 | 0.68421053 |
| chr18: 63207225 | 0.889706 | 16.8640183 | 0.8125 | 0.91176471 |
| chr9: 134772536 | 0.88952 | 69.6691176 | 0.95454545 | 0.69444444 |
| chr8: 38495018 | 0.889452 | 89.3820225 | 0.86206897 | 0.76470588 |
| chr13: 112107447 | 0.889401 | 89.4444444 | 0.92857143 | 0.74193548 |
| chr5: 100864671 | 0.889368 | 86.6071429 | 0.75 | 0.93103448 |
| chr14: 91418510 | 0.889286 | 1.26814388 | 0.875 | 0.82857143 |
| chr9: 134768753 | 0.889277 | 70.3546454 | 0.84615385 | 0.87878788 |
| chr19: 13245332 | 0.889247 | 89.5121951 | 0.93333333 | 0.83870968 |
| chr11: 47327256 | 0.889216 | 46.1757991 | 0.93333333 | 0.79411765 |
| chr8: 42275934 | 0.889216 | 92.7248677 | 0.86666667 | 0.79411765 |
| chr12: 103165897 | 0.889205 | 91.8103448 | 0.75 | 1 |
| chr18: 38714972 | 0.88914 | 26.6666667 | 0.69230769 | 1 |
| chr7: 157824560 | 0.888889 | 52.7777778 | 0.86666667 | 0.84848485 |
| chr14: 75978696 | 0.888889 | 77.7472527 | 0.96153846 | 0.75555556 |
| chr15: 40873466 | 0.888889 | 29.1503268 | 0.85185185 | 0.81632653 |
| chr4: 104663323 | 0.888889 | 88.1944444 | 0.94444444 | 0.74285714 |
| chr5: 1316150 | 0.888655 | 3.00975709 | 0.85714286 | 0.91176471 |
| chr4: 189096959 | 0.888571 | 87.8676471 | 0.8 | 0.85714286 |
| chr9: 134750430 | 0.888571 | 81.3186813 | 1 | 0.77142857 |
| chr13: 98405890 | 0.888542 | 30.2419355 | 0.8 | 0.90625 |
| chr9: 134825870 | 0.888386 | 38.3152174 | 0.88235294 | 0.74358974 |
| chr16: 52621839 | 0.888235 | 60.8152174 | 0.93333333 | 0.82352941 |
| chr17: 62653135 | 0.888235 | 0.24769007 | 0.86666667 | 0.85294118 |
| chr9: 134881575 | 0.888235 | 25.8333333 | 0.86666667 | 0.82352941 |
| chr7: 157633794 | 0.888112 | 53.6873156 | 0.84615385 | 1 |
| chr9: 114170237 | 0.8881 | 74.5726496 | 0.82758621 | 0.8627451 |
| chr18: 77020828 | 0.888009 | 85.1648352 | 0.92307692 | 0.70588235 |
| chr2: 216840164 | 0.887955 | 93.0952381 | 0.92857143 | 0.7254902 |
| chr2: 181684626 | 0.887879 | 71.1111111 | 1 | 0.72727273 |
| chr9: 134819274 | 0.887879 | 79.1666667 | 0.93333333 | 0.78787879 |
| chr21: 34743983 | 0.887771 | 73.4408602 | 0.94117647 | 0.73684211 |
| chr17: 57874702 | 0.887762 | 35.4761905 | 0.62068966 | 1 |
| chr22: 28923959 | 0.887534 | 4.63800905 | 0.94444444 | 0.73170732 |
| chr21: 44698714 | 0.88748 | 88.7152778 | 0.92307692 | 0.78723404 |
| chr11: 65551324 | 0.887424 | 12.2222222 | 0.72413793 | 0.92156863 |
| chr9: 113593972 | 0.887273 | 71.8253968 | 0.76 | 0.90909091 |
| chr7: 2193759 | 0.887255 | 81.6498316 | 0.73333333 | 0.94117647 |
| chr6: 168440498 | 0.887122 | 10.7736761 | 0.70588235 | 0.94594595 |
| chr8: 141264221 | 0.887037 | 66.9871795 | 0.92592593 | 0.8 |
| chr16: 4260115 | 0.887019 | 86.2229102 | 0.92307692 | 0.8125 |
| chr16: 4537502 | 0.887019 | 71.8253968 | 0.8125 | 0.82051282 |
| chr20: 20365491 | 0.886997 | 7.70308123 | 1 | 0.73684211 |
| chr1: 7491237 | 0.886869 | 22.9223901 | 0.86666667 | 0.93939394 |
| chr20: 20364647 | 0.886667 | 1.2112473 | 0.86666667 | 0.82857143 |
| chr11: 31893506 | 0.886555 | 58.9285714 | 1 | 0.73529412 |
| chr19: 41704632 | 0.886555 | 58.3751793 | 0.92857143 | 0.76470588 |
| chr20: 17068564 | 0.886555 | 66.3531998 | 0.71428571 | 0.94117647 |
| chr5: 6543207 | 0.886555 | 78.8888889 | 0.78571429 | 0.88235294 |
| chr19: 41323849 | 0.886458 | 47.7272727 | 1 | 0.6875 |
| chr2: 85733778 | 0.886458 | 79.3706294 | 0.79166667 | 0.9 |
| chr7: 157685012 | 0.886409 | 75.7440476 | 0.875 | 0.78571429 |
| chr11: 75240135 | 0.886364 | 78.4722222 | 0.85714286 | 0.90909091 |
| chr6: 168315218 | 0.886305 | 43.2748538 | 0.74074074 | 0.90697674 |
| chr14: 64633473 | 0.886285 | 85.2429297 | 0.875 | 0.80555556 |
| chr11: 20056293 | 0.886275 | 40.5882353 | 0.86666667 | 0.91176471 |
| chr14: 44379797 | 0.886161 | 75.7019969 | 0.85714286 | 0.8125 |
| chr16: 4537618 | 0.886029 | 30.075188 | 0.82352941 | 0.9 |
| chr7: 157864088 | 0.886029 | 74.9285714 | 0.75 | 0.94117647 |
| chr10: 133309864 | 0.885862 | 8.86958387 | 0.86206897 | 0.8 |
| chr14: 88256419 | 0.885859 | 94.5906433 | 0.93333333 | 0.81818182 |
| chr16: 19425106 | 0.885859 | 37.9734848 | 0.73333333 | 0.96969697 |
| chr6: 168355227 | 0.885835 | 79.4736842 | 0.77272727 | 0.93023256 |
| chr9: 134765734 | 0.885714 | 64.4736842 | 1 | 0.77142857 |
| chr6: 168453036 | 0.885714 | 71.9607843 | 0.86666667 | 0.8 |
| chr21: 41490949 | 0.88558 | 31.8181818 | 0.72727273 | 0.93103448 |
| chr7: 73989381 | 0.885504 | 28.507384 | 0.92857143 | 0.79411765 |
| chr12: 3077894 | 0.885488 | 33.0265685 | 0.92592593 | 0.79591837 |
| chr1: 53462420 | 0.885417 | 89.8989899 | 0.875 | 0.81818182 |
| chr9: 113593928 | 0.885417 | 86.6071429 | 0.86666667 | 0.78125 |
| chr10: 133309740 | 0.885326 | 36.161435 | 0.69565217 | 0.95 |
| chr7: 101666174 | 0.885294 | 44.077621 | 0.86666667 | 0.82352941 |
| chr11: 14974476 | 0.885045 | 74.7727273 | 0.92857143 | 0.78125 |
| chr5: 100944956 | 0.884848 | 66.9642857 | 0.73333333 | 0.96969697 |
| chr20: 22557482 | 0.884821 | 85.4152824 | 0.875 | 0.91428571 |
| chr18: 74782855 | 0.884615 | 91.1067194 | 0.72727273 | 1 |
| chr19: 3656386 | 0.884444 | 92.5824176 | 0.81481481 | 0.88 |
| chr7: 26397761 | 0.884375 | 64.3858478 | 0.8 | 0.9375 |
| chr19: 13463491 | 0.884314 | 67.556719 | 0.8 | 0.85294118 |
| chr22: 38087144 | 0.884314 | 75 | 0.73333333 | 0.91176471 |
| chr9: 130535984 | 0.884314 | 0.93338069 | 0.93333333 | 0.85294118 |
| chr9: 134892203 | 0.884314 | 35.4444444 | 1 | 0.67647059 |
| chr13: 107429178 | 0.884199 | 94.3939394 | 0.85714286 | 0.81818182 |
| chr19: 38253194 | 0.884199 | 39.8275862 | 0.76190476 | 0.90909091 |
| chr17: 82078952 | 0.884164 | 82.5757576 | 0.81818182 | 0.87096774 |
| chr1: 1174126 | 0.883987 | 94.6428571 | 0.94117647 | 0.75 |
| chr1: 232307467 | 0.883929 | 85.4497354 | 1 | 0.71875 |
| chr6: 14499964 | 0.883929 | 66.9642857 | 0.91666667 | 0.89285714 |
| chr16: 4260566 | 0.883657 | 32.7380952 | 0.78947368 | 0.89473684 |
| chr16: 85286422 | 0.883556 | 99.4897959 | 0.92 | 0.77777778 |
| chr17: 74006568 | 0.883553 | 81.9293478 | 0.8 | 0.86842105 |
| chr7: 101707772 | 0.883523 | 15.6923077 | 1 | 0.84375 |
| chr6: 168496991 | 0.883484 | 77.7047414 | 0.84615385 | 0.91176471 |
| chr18: 27786339 | 0.883403 | 14.9339301 | 0.78571429 | 0.85294118 |
| chr13: 113793557 | 0.883333 | 92.9802956 | 0.93333333 | 0.8 |
| chr19: 47209566 | 0.883333 | 84.5 | 0.86666667 | 0.85294118 |
| chr2: 113325550 | 0.883333 | 59.8039216 | 0.8 | 0.85294118 |
| chr5: 176529022 | 0.883333 | 89.1176471 | 0.86666667 | 0.79411765 |
| chr8: 52150549 | 0.883333 | 83.75 | 0.8 | 0.94117647 |
| chr9: 113594013 | 0.883182 | 49.3589744 | 0.84 | 0.81818182 |
| chr9: 134490841 | 0.883117 | 86.0576923 | 0.85714286 | 0.90909091 |
| chr7: 1620862 | 0.883108 | 57.6705882 | 0.7 | 0.97297297 |
| chr9: 134179005 | 0.883107 | 15.5152672 | 0.94117647 | 0.87179487 |
| chr15: 63053313 | 0.882883 | 76.6304348 | 0.77777778 | 0.94594595 |
| chr12: 124617614 | 0.882828 | 20.7142857 | 0.93333333 | 0.72727273 |
| chr21: 45032480 | 0.882828 | 57.5554773 | 0.73333333 | 0.93939394 |
| chr13: 113723025 | 0.882813 | 58.4770115 | 0.92857143 | 0.71875 |
| chr7: 150945169 | 0.882813 | 76.3888889 | 0.9375 | 0.75 |
| chr12: 6961918 | 0.882796 | 8.39160839 | 0.8 | 0.90322581 |
| chr19: 39307859 | 0.882407 | 16.2280702 | 0.875 | 0.8 |
| chr16: 89948104 | 0.882353 | 1.77343619 | 0.88235294 | 0.78787879 |
| chr19: 8153491 | 0.882353 | 56.9354839 | 0.73333333 | 0.91176471 |
| chr20: 20365487 | 0.882353 | 8.11688312 | 0.94117647 | 0.73684211 |
| chr20: 38294974 | 0.882353 | 68.627451 | 0.8 | 0.85294118 |
| chr3: 73790643 | 0.882353 | 92.3001311 | 0.92857143 | 0.73529412 |
| chr6: 153237290 | 0.882353 | 67.6435407 | 0.73333333 | 0.91176471 |
| chr6: 168130594 | 0.882353 | 83.7985742 | 0.8 | 0.94117647 |
| chr7: 2524564 | 0.882353 | 15.7198306 | 0.8 | 0.88235294 |
| chr9: 134785301 | 0.882353 | 67.9487179 | 0.94117647 | 0.8 |
| chr9: 136367817 | 0.882353 | 52.7777778 | 0.86206897 | 0.80392157 |
| chr19: 38253176 | 0.882325 | 43.4734735 | 0.86956522 | 0.7826087 |
| chr19: 406561 | 0.882212 | 86.6071429 | 0.92307692 | 0.8125 |
| chr1: 52633297 | 0.882143 | 6.94444444 | 0.8 | 0.92857143 |
| chr9: 93819344 | 0.882125 | 80.9975603 | 0.72413793 | 0.97959184 |
| chr11: 76784948 | 0.882102 | 49.2424242 | 0.83333333 | 0.86363636 |
| chr14: 36524358 | 0.882086 | 72.4747475 | 0.85185185 | 0.81632653 |
| chr13: 112106739 | 0.882035 | 87.75 | 0.92857143 | 0.78787879 |
| chr6: 157228665 | 0.8819 | 80.2175665 | 0.78947368 | 0.87804878 |
| chr7: 157953525 | 0.881875 | 77.3504274 | 0.85 | 0.85 |
| chr10: 101780858 | 0.881818 | 91.2878788 | 0.86666667 | 0.78787879 |
| chr12: 110942244 | 0.881818 | 35.4166667 | 0.86666667 | 0.87878788 |
| chr6: 43578006 | 0.881766 | 95.5824972 | 0.94444444 | 0.71794872 |
| chr2: 51027486 | 0.88172 | 97.4804382 | 0.93333333 | 0.77419355 |
| chr7: 157685871 | 0.881506 | 62.5567151 | 0.71428571 | 1 |
| chr21: 45032448 | 0.881434 | 40.3947368 | 0.75 | 0.91176471 |
| chr21: 45032452 | 0.881434 | 59.8684211 | 0.75 | 0.88235294 |
| chr7: 157017829 | 0.881422 | 17.3118548 | 0.75862069 | 0.89795918 |
| chr11: 65551413 | 0.881373 | 44.5175439 | 0.73333333 | 0.91176471 |
| chr7: 157544388 | 0.881303 | 84.8739496 | 0.78571429 | 0.91176471 |
| chr6: 50845964 | 0.881222 | 47.8103176 | 0.84615385 | 0.85294118 |
| chr5: 138650055 | 0.881169 | 73.6111111 | 0.86363636 | 0.8 |
| chr1: 229147368 | 0.881081 | 88.4444444 | 0.86666667 | 0.83783784 |
| chr9: 134782536 | 0.881076 | 19.375 | 0.95833333 | 0.79166667 |
| chr9: 134316337 | 0.880878 | 82.2134387 | 1 | 0.65517241 |
| chr12: 124259764 | 0.880831 | 30.2173913 | 0.82142857 | 0.85714286 |
| chr11: 11578685 | 0.880808 | 75.1282051 | 0.93333333 | 0.81818182 |
| chr20: 17068489 | 0.880808 | 94.8684211 | 0.86666667 | 0.84848485 |
| chr20: 20365493 | 0.880805 | 4.23280423 | 1 | 0.73684211 |
| chr22: 28923940 | 0.880759 | 9.90338164 | 0.83333333 | 0.87804878 |
| chr22: 28923966 | 0.880759 | 15.0323774 | 0.77777778 | 0.87804878 |
| chr1: 153536136 | 0.880682 | 1.61290323 | 0.90909091 | 0.75 |
| chr9: 93861253 | 0.880435 | 85.1648352 | 0.8 | 0.91304348 |
| chr11: 64540473 | 0.880392 | 47.7272727 | 0.73333333 | 0.91176471 |
| chr2: 229672283 | 0.880392 | 59.6590909 | 0.86666667 | 0.79411765 |
| chr7: 157633759 | 0.880392 | 74.2946708 | 0.86666667 | 0.91176471 |
| chr9: 134534796 | 0.880392 | 43.0262046 | 0.86666667 | 0.85294118 |
| chr9: 114170276 | 0.880342 | 27.5362319 | 1 | 0.71794872 |
| chr19: 5950505 | 0.880303 | 89.1812865 | 0.72727273 | 0.93333333 |
| chr1: 32885121 | 0.880252 | 32.0512821 | 0.64285714 | 0.97058824 |
| chr12: 6962651 | 0.880252 | 7.10784314 | 0.85714286 | 0.76470588 |
| chr18: 27786338 | 0.880252 | 16.5158371 | 0.92857143 | 0.79411765 |
| chr18: 63207261 | 0.880252 | 7.73809524 | 0.85714286 | 0.82352941 |
| chr19: 41704681 | 0.880252 | 23.4280792 | 0.78571429 | 0.91176471 |
| chr8: 141289585 | 0.880252 | 62.5874126 | 0.78571429 | 1 |
| chr11: 124895535 | 0.880208 | 40.8730159 | 0.95833333 | 0.75 |
| chr17: 57874844 | 0.880124 | 66.2284546 | 0.7826087 | 0.91428571 |
| chr11: 75335191 | 0.88 | 71.7482517 | 0.67857143 | 0.98 |
| chr18: 71337086 | 0.88 | 11.1742424 | 0.86666667 | 0.8 |
| chr2: 46983412 | 0.879939 | 84.6989967 | 0.89285714 | 0.85106383 |
| chr1: 212589163 | 0.87987 | 42.1296296 | 0.85714286 | 0.81818182 |
| chr20: 48751499 | 0.87987 | 59.0909091 | 0.85714286 | 0.81818182 |
| chr4: 78056611 | 0.879798 | 0.67241038 | 0.86666667 | 0.84848485 |
| chr3: 176814702 | 0.879658 | 86.6025641 | 0.78571429 | 0.93478261 |
| chr17: 81246299 | 0.879639 | 81.3852814 | 0.7826087 | 0.92682927 |
| chr2: 3940818 | 0.879596 | 40.5837344 | 0.75 | 0.97058824 |
| chr12: 98238503 | 0.879583 | 81.4646465 | 0.8 | 0.875 |
| chr9: 114170300 | 0.879487 | 50.228833 | 0.93333333 | 0.71794872 |
| chr11: 924647 | 0.879424 | 81.9264069 | 0.88888889 | 0.84444444 |
| chr19: 55254024 | 0.879412 | 46.1061947 | 0.86666667 | 0.79411765 |
| chr2: 208703401 | 0.879412 | 84.8728814 | 0.86666667 | 0.82352941 |
| chr20: 22598941 | 0.879412 | 92.2619048 | 0.8 | 0.91176471 |

TABLE 28
DNA methylation sites associated with SCLC-N

| MethylationSite | AUC | threshold | specificity | sensitivity |
|---|---|---|---|---|
| chr1:54356529 | 1 | 7.56298908 | 1 | 1 |
| chr1:109296245 | 1 | 66.0714286 | 1 | 1 |
| chr1:109296247 | 1 | 78.021978 | 1 | 1 |
| chr1:220653171 | 1 | 48.9473684 | 1 | 1 |
| chr1:220653195 | 1 | 53.5028249 | 1 | 1 |
| chr11:35853956 | 1 | 69.047619 | 1 | 1 |
| chr11:64154503 | 1 | 37.0689655 | 1 | 1 |
| chr11:64154571 | 1 | 52.8287462 | 1 | 1 |
| chr11:64154574 | 1 | 68.75 | 1 | 1 |
| chr12:108792675 | 1 | 60.3896104 | 1 | 1 |
| chr17:72440313 | 1 | 32.0175439 | 1 | 1 |
| chr17:72440361 | 1 | 43.3035714 | 1 | 1 |
| chr2:223901322 | 1 | 60.4609929 | 1 | 1 |
| chr20:19919227 | 1 | 59.6972564 | 1 | 1 |
| chr3:52468694 | 1 | 44.5856354 | 1 | 1 |
| chr5:160245925 | 1 | 26.3241625 | 1 | 1 |
| chr6:44261803 | 1 | 68.1818182 | 1 | 1 |
| chr8:42275934 | 1 | 70.5376344 | 1 | 1 |
| chr9:131721310 | 1 | 34.5744681 | 1 | 1 |
| chr9:131721341 | 1 | 72.3169192 | 1 | 1 |
| chr9:131721355 | 1 | 28.125 | 1 | 1 |
| chr9:131721389 | 1 | 61.030303 | 1 | 1 |
| chr2:216909627 | 0.998476 | 43.9102564 | 0.97560976 | 1 |
| chr20:19919173 | 0.996951 | 58.5885886 | 0.97560976 | 1 |
| chr9:131721356 | 0.996951 | 51.8405797 | 0.97560976 | 1 |
| chr22:50276667 | 0.996795 | 63.3012821 | 0.97435897 | 1 |
| chr17:72440360 | 0.996711 | 46.422956 | 0.97368421 | 1 |
| chr6:37537738 | 0.996599 | 28.5714286 | 0.97619048 | 1 |
| chr1:220653165 | 0.996516 | 55.6140351 | 0.97560976 | 1 |
| chr1:220653180 | 0.996516 | 58.245614 | 0.97560976 | 1 |
| chr8:140270696 | 0.996516 | 63.8045541 | 0.97560976 | 1 |
| chr12:108792684 | 0.996429 | 74.6031746 | 0.975 | 1 |
| chr19:3656274 | 0.995556 | 59.1269841 | 0.95555556 | 1 |
| chr1:54356527 | 0.995192 | 7.07146427 | 0.97435897 | 1 |
| chr19:47650943 | 0.995192 | 70.375 | 0.97435897 | 1 |
| chr7:149261799 | 0.995098 | 12.1428571 | 0.97058824 | 1 |
| chr2:216380208 | 0.994709 | 23.5429448 | 0.95238095 | 1 |
| chr17:72440372 | 0.993902 | 21.8781598 | 0.97560976 | 1 |
| chr9:131721340 | 0.993902 | 87.9503546 | 0.95121951 | 1 |
| chr9:131721342 | 0.993902 | 87.3670213 | 0.95121951 | 1 |
| chr2:216380274 | 0.99375 | 23.5663082 | 0.975 | 1 |
| chr11:67493309 | 0.99359 | 79.5833333 | 0.94871795 | 1 |
| chr2:65583301 | 0.993243 | 71.0164835 | 0.94594595 | 1 |
| chr5:160245988 | 0.993243 | 47.7272727 | 0.94594595 | 1 |
| chr3:52468622 | 0.992674 | 50 | 0.94871795 | 1 |
| chr17:74823299 | 0.992593 | 86.0576923 | 0.95555556 | 1 |
| chr1:160362850 | 0.992188 | 23.8636364 | 0.95 | 1 |
| chr12:31009628 | 0.991071 | 35.625 | 0.92857143 | 1 |
| chr14:64633473 | 0.990956 | 55 | 0.97674419 | 1 |
| chr18:59049918 | 0.990854 | 18.414548 | 0.97560976 | 1 |
| chr20:63878908 | 0.990854 | 62.6436782 | 0.92682927 | 1 |
| chr5:16538225 | 0.990854 | 55.3655713 | 0.92682927 | 1 |
| chr6:19719869 | 0.990854 | 56.7176871 | 0.92682927 | 1 |
| chr6:34128215 | 0.990854 | 73.0952381 | 0.97560976 | 1 |
| chr6:34128233 | 0.990854 | 79.1666667 | 0.97560976 | 1 |
| chr1:109296235 | 0.990625 | 43.0960086 | 0.95 | 1 |
| chr3:52522225 | 0.990625 | 39.3939394 | 0.95 | 1 |
| chr8:134941130 | 0.990625 | 64.2857143 | 0.925 | 1 |
| chr7:149261769 | 0.990196 | 30.3571429 | 0.97058824 | 1 |
| chr19:3656352 | 0.990018 | 42.6406926 | 0.94827586 | 0.94736842 |
| chr2:85733778 | 0.989987 | 58.7121212 | 0.95744681 | 0.94117647 |
| chr4:850796 | 0.989286 | 85.2071006 | 0.925 | 1 |
| chr3:126013960 | 0.988722 | 57.9831933 | 0.97368421 | 1 |
| chr19:3655899 | 0.988372 | 32.8282828 | 0.93023256 | 1 |
| chr22:44049853 | 0.988176 | 67.0192308 | 0.94594595 | 1 |
| chr9:120242580 | 0.987879 | 46.9008264 | 0.95555556 | 1 |
| chr1:41657833 | 0.987805 | 80.9090909 | 0.90243902 | 1 |
| chr1:220653181 | 0.987805 | 82.5757576 | 0.90243902 | 1 |
| chr11:8239208 | 0.987805 | 32.3643411 | 0.97560976 | 1 |
| chr2:65583300 | 0.987805 | 61.7647059 | 0.95121951 | 1 |
| chr5:16538207 | 0.987805 | 55.8731855 | 0.92682927 | 1 |
| chr6:34128222 | 0.987805 | 73.8095238 | 0.97560976 | 1 |
| chr9:131721343 | 0.987805 | 85.7777778 | 0.95121951 | 1 |
| chr7:149261778 | 0.987745 | 38.0952381 | 0.94117647 | 1 |
| chr14:105178678 | 0.9875 | 64.6381579 | 0.925 | 1 |
| chr12:31009602 | 0.987179 | 38.1619938 | 0.97435897 | 1 |
| chr1:28869236 | 0.986111 | 20.9183673 | 0.98275862 | 1 |
| chr1:220653223 | 0.986063 | 82.3984526 | 0.90243902 | 1 |
| chr7:44254153 | 0.986063 | 37.5718454 | 0.95121951 | 1 |
| chr6:43475701 | 0.984756 | 26.2531861 | 0.97560976 | 1 |
| chr15:68347588 | 0.984375 | 67.9060665 | 0.925 | 1 |
| chr20:49345755 | 0.983232 | 16.3605642 | 0.97560976 | 1 |
| chr5:160245950 | 0.983108 | 42.0761671 | 0.89189189 | 1 |
| chr19:3656298 | 0.982222 | 67.4242424 | 0.93333333 | 1 |
| chr19:3655920 | 0.981912 | 24.5384615 | 1 | 0.88888889 |
| chr20:49345760 | 0.981707 | 17.8739348 | 0.95121951 | 1 |
| chr6:34128216 | 0.981707 | 85.4166667 | 0.95121951 | 1 |
| chr3:52468649 | 0.981685 | 33.7037037 | 0.97435897 | 1 |
| chr1:944700 | 0.98125 | 12.7717391 | 0.9 | 1 |
| chr1:54356503 | 0.98125 | 22.3680124 | 0.9 | 1 |
| chr1:54356526 | 0.98125 | 20.11604 | 0.95 | 1 |
| chr1:54356528 | 0.98125 | 21.4285714 | 0.925 | 1 |
| chr2:216909691 | 0.98125 | 75.7142857 | 0.9 | 1 |
| chr5:160245949 | 0.980769 | 15.6059768 | 1 | 0.875 |
| chr21:41490949 | 0.980392 | 13.5714286 | 0.97058824 | 1 |
| chr17:50076230 | 0.980263 | 64.9616368 | 0.94736842 | 1 |
| chr2:25702014 | 0.979688 | 36.6898148 | 0.95 | 1 |
| chr19:3655770 | 0.979167 | 57.4074074 | 0.92592593 | 1 |
| chr9:120242557 | 0.979167 | 57.8495562 | 0.95833333 | 1 |
| chr16:85439239 | 0.978723 | 60.8990148 | 0.89361702 | 1 |
| chr1:53320062 | 0.978659 | 75.7142857 | 1 | 0.875 |
| chr5:16538206 | 0.978659 | 29.3956044 | 1 | 0.875 |
| chr6:19719865 | 0.978659 | 17.1929825 | 1 | 0.875 |
| chr11:855989 | 0.978571 | 62.4675325 | 0.975 | 1 |
| chr19:3655921 | 0.978405 | 43.9912281 | 0.88372093 | 1 |
| chr6:107633786 | 0.97832 | 87.4632233 | 0.87804878 | 1 |
| chr19:14182660 | 0.978125 | 73.7938596 | 0.9 | 1 |
| chr19:48350598 | 0.978022 | 38.75 | 0.92307692 | 1 |
| chr3:52468650 | 0.978022 | 15.7575758 | 0.97435897 | 1 |
| chr16:10622418 | 0.977941 | 64.5833333 | 0.97058824 | 1 |
| chr3:52468678 | 0.977564 | 46.3142219 | 0.92307692 | 1 |
| chr11:20055879 | 0.977352 | 86.0062893 | 0.95121951 | 1 |
| chr2:216909621 | 0.977134 | 74.6848739 | 0.90243902 | 1 |
| chr6:34128193 | 0.977134 | 63.8713745 | 0.97560976 | 1 |
| chr16:85287295 | 0.976916 | 70.3756881 | 0.85964912 | 1 |
| chr1:53320061 | 0.976744 | 84.4129555 | 0.90697674 | 1 |
| chr7:100890535 | 0.97619 | 28.7828947 | 0.97619048 | 1 |
| chr12:48675723 | 0.97561 | 79.0117417 | 0.90243902 | 1 |
| chr2:25701951 | 0.97561 | 83.1845238 | 0.87804878 | 1 |
| chr20:63835624 | 0.97561 | 28.4090909 | 0.90243902 | 1 |
| chr4:76612797 | 0.97561 | 69.6969697 | 0.95121951 | 1 |
| chr6:43475769 | 0.97561 | 34.6153846 | 0.97560976 | 1 |
| chr3:16132696 | 0.975 | 18.3969466 | 0.925 | 1 |
| chr11:66866493 | 0.974359 | 24.1808133 | 0.94871795 | 0.9 |
| chr12:31009572 | 0.974359 | 31.1121857 | 0.92307692 | 1 |
| chr1:226588954 | 0.974069 | 67.3611111 | 0.87234043 | 1 |
| chr16:66960251 | 0.973958 | 85.3571429 | 0.89583333 | 0.9375 |
| chr19:3656369 | 0.973635 | 60.8242304 | 0.94915254 | 0.94444444 |
| chr17:4809230 | 0.973545 | 52.8971029 | 0.88095238 | 1 |
| chr7:157018053 | 0.973545 | 25.8333333 | 0.88095238 | 1 |
| chr20:11271495 | 0.973238 | 67.4459459 | 0.88135593 | 1 |
| chr17:82001560 | 0.973214 | 72.1153846 | 0.9047619 | 1 |
| chr17:82001559 | 0.972561 | 85.1818182 | 0.87804878 | 1 |
| chr2:85770228 | 0.972561 | 57.8703704 | 0.95121951 | 1 |
| chr20:49345801 | 0.972561 | 26.2962963 | 0.90243902 | 1 |
| chr21:41458778 | 0.972561 | 89.9541284 | 0.90243902 | 1 |
| chr6:34128249 | 0.972561 | 94.2788686 | 0.87804878 | 1 |
| chr11:44963230 | 0.972222 | 55.0480769 | 0.94827586 | 0.94444444 |
| chr1:944730 | 0.971875 | 7.29166667 | 1 | 0.875 |
| chr12:108792716 | 0.971875 | 71.7839375 | 0.9 | 1 |
| chr22:44049787 | 0.971875 | 61.4709852 | 0.925 | 1 |
| chr3:52522201 | 0.971875 | 79.1666667 | 0.925 | 1 |
| chr4:6308154 | 0.971602 | 47.5334448 | 0.96551724 | 0.94117647 |
| chr2:85733793 | 0.971214 | 50.1602564 | 0.93617021 | 0.94117647 |
| chr19:12953781 | 0.971154 | 7.91105671 | 0.92307692 | 1 |
| chr1:43550353 | 0.970588 | 88.75 | 0.94117647 | 1 |
| chr21:41490965 | 0.970588 | 3.57142857 | 0.94117647 | 1 |
| chr12:108792685 | 0.970313 | 86.1904762 | 0.875 | 1 |
| chr3:16132702 | 0.970313 | 18.2194617 | 0.9 | 1 |
| chr1:15723882 | 0.969925 | 67.0138889 | 0.86842105 | 1 |
| chr1:54356555 | 0.969551 | 19.375 | 0.92307692 | 1 |
| chr12:130484433 | 0.969512 | 35.0877193 | 0.92682927 | 1 |
| chr17:4809161 | 0.969512 | 55.4824561 | 0.97560976 | 0.875 |
| chr6:19719861 | 0.969512 | 34.4170772 | 0.97560976 | 0.875 |
| chr6:26385134 | 0.969512 | 84.6927966 | 0.97560976 | 0.875 |
| chr6:168130602 | 0.969512 | 82.8571429 | 0.95121951 | 1 |
| chr19:13913675 | 0.969444 | 80.8823529 | 0.86666667 | 1 |
| chr16:85287080 | 0.969298 | 29.5833333 | 0.8 | 1 |
| chr11:888785 | 0.969231 | 91.9871795 | 0.82 | 1 |
| chr19:3655898 | 0.968992 | 55.3977273 | 0.86046512 | 1 |
| chr1:1921168 | 0.967949 | 67.4242424 | 0.84615385 | 1 |
| chr2:200087052 | 0.967949 | 38.0986938 | 0.92307692 | 1 |
| chr19:2354832 | 0.967901 | 90.1576684 | 0.82222222 | 1 |
| chr11:129699789 | 0.967857 | 76.3931889 | 0.85 | 1 |
| chr1:28869229 | 0.967433 | 43.6507937 | 0.94827586 | 1 |
| chr19:18422970 | 0.967376 | 71.1111111 | 0.93617021 | 0.86666667 |
| chr1:944737 | 0.967188 | 4.16666667 | 0.975 | 0.875 |
| chr22:44049781 | 0.967188 | 68.9370485 | 0.9 | 1 |
| chr20:25239829 | 0.966667 | 86.987782 | 0.91666667 | 0.94736842 |
| chr9:120242569 | 0.966667 | 57.9022989 | 0.95833333 | 0.9 |
| chr6:148509695 | 0.96657 | 9.42982456 | 0.97674419 | 0.875 |
| chr1:153536157 | 0.966216 | 24.047619 | 0.94594595 | 1 |
| chr9:120242617 | 0.966071 | 55.2380952 | 0.875 | 1 |
| chr12:31009603 | 0.965774 | 55.2255639 | 0.9047619 | 1 |
| chr18:62333487 | 0.965608 | 29.8469388 | 0.97619048 | 0.88888889 |
| chr8:140270737 | 0.965157 | 68.8577586 | 0.97560976 | 0.85714286 |
| chr19:3655888 | 0.965116 | 39.6011396 | 0.90697674 | 1 |
| chr17:82001529 | 0.964939 | 31.9123601 | 0.92682927 | 1 |
| chr7:102424329 | 0.964939 | 86.1904762 | 0.92682927 | 0.875 |
| chr2:200087071 | 0.964744 | 44.1520468 | 0.8974359 | 1 |
| chr16:85287095 | 0.964474 | 47.0208333 | 0.83333333 | 1 |
| chr11:855972 | 0.964286 | 83.7792461 | 0.925 | 1 |
| chr11:855981 | 0.964286 | 50.6097561 | 0.925 | 1 |
| chr9:120242622 | 0.964286 | 5.55555556 | 0.975 | 0.85714286 |
| chr19:3656250 | 0.964115 | 79.3706294 | 0.90909091 | 0.94736842 |
| chr20:19836028 | 0.964063 | 31.1987705 | 0.975 | 0.875 |
| chr1:944699 | 0.963816 | 6.10795455 | 0.97368421 | 0.875 |
| chr17:2418940 | 0.963816 | 83.0143541 | 0.86842105 | 1 |
| chr3:185397845 | 0.963816 | 77.3504274 | 0.81578947 | 1 |
| chr7:44151120 | 0.963508 | 80.9090909 | 0.83333333 | 0.94117647 |
| chr11:35853892 | 0.963415 | 66.3793103 | 0.87804878 | 1 |
| chr12:123253744 | 0.963415 | 87.8676471 | 0.90243902 | 1 |
| chr20:49345788 | 0.963415 | 10.8930864 | 0.90243902 | 1 |
| chr6:34128234 | 0.963415 | 72.2222222 | 0.97560976 | 0.875 |
| chr7:750747 | 0.963415 | 31.127451 | 1 | 0.875 |
| chr2:205764439 | 0.963141 | 31.40625 | 0.8974359 | 1 |
| chr18:59050139 | 0.962963 | 73.030303 | 0.87037037 | 0.94117647 |
| chr5:172103442 | 0.962798 | 54.8717949 | 0.88095238 | 1 |
| chr9:120242544 | 0.9625 | 65.1515152 | 0.875 | 1 |
| chr1:28869207 | 0.962165 | 27.6794035 | 0.87931034 | 1 |
| chr16:11997774 | 0.961794 | 89.4444444 | 0.88372093 | 1 |
| chr8:134499858 | 0.961749 | 78.3482143 | 1 | 0.88888889 |
| chr17:29158240 | 0.961672 | 85.2419355 | 0.85365854 | 1 |
| chr1:54356559 | 0.961538 | 14.2294713 | 0.92307692 | 1 |
| chr6:34545466 | 0.96131 | 61.5196078 | 0.88095238 | 1 |
| chr8:134941060 | 0.961111 | 49.137931 | 0.825 | 1 |
| chr10:101780858 | 0.960938 | 87.75 | 0.85 | 1 |
| chr19:12953855 | 0.960938 | 12.3737374 | 0.9 | 1 |
| chr3:53702770 | 0.960714 | 81.3580247 | 0.9 | 1 |
| chr12:56997304 | 0.960366 | 60.2955665 | 0.97560976 | 0.875 |
| chr6:34128223 | 0.960366 | 75 | 0.97560976 | 0.875 |
| chr18:77053845 | 0.960317 | 87.3015873 | 0.92857143 | 0.88888889 |
| chr20:22557408 | 0.960227 | 60.2564103 | 0.90909091 | 1 |
| chr6:110414306 | 0.96 | 65.1515152 | 0.88 | 1 |
| chr9:136013178 | 0.96 | 76.8421053 | 0.91428571 | 1 |
| chr1:230759612 | 0.959936 | 47.008547 | 0.92307692 | 1 |
| chr6:44261814 | 0.959936 | 86.1904762 | 0.87179487 | 1 |
| chr6:42963345 | 0.959472 | 70.094086 | 0.91803279 | 0.88888889 |
| chr9:95738191 | 0.959375 | 69.8591549 | 0.9 | 1 |
| chr16:85355101 | 0.95935 | 78.4615385 | 0.87804878 | 1 |
| chr19:2354863 | 0.959302 | 81.6666667 | 0.90697674 | 1 |
| chr1:53320016 | 0.959302 | 20.0910931 | 0.90697674 | 1 |
| chr16:70318894 | 0.958929 | 89.6057348 | 0.925 | 0.85714286 |
| chr16:85696894 | 0.958333 | 83.2329317 | 0.78333333 | 1 |
| chr17:82001584 | 0.958333 | 12.5966944 | 0.95238095 | 0.875 |
| chr2:200087032 | 0.958333 | 48.8529015 | 0.8974359 | 1 |
| chr7:128075308 | 0.958333 | 69.6153846 | 0.92105263 | 1 |
| chr1:32885121 | 0.958188 | 11.9767442 | 0.97560976 | 0.85714286 |
| chr21:41665312 | 0.958188 | 47.7272727 | 0.87804878 | 1 |
| chr7:128881062 | 0.957955 | 46.875 | 0.85454545 | 1 |
| chr6:169160984 | 0.957849 | 54.5694502 | 0.88372093 | 1 |
| chr3:196992182 | 0.957722 | 63.7934603 | 0.91803279 | 0.94736842 |
| chr6:40349319 | 0.957672 | 18.7280702 | 0.95238095 | 0.88888889 |
| chr7:44151126 | 0.957639 | 26.1363636 | 0.95833333 | 0.86666667 |
| chr1:54356214 | 0.957483 | 38.8560158 | 0.97619048 | 0.85714286 |
| chr7:5276684 | 0.957447 | 40.6565657 | 0.95744681 | 0.88888889 |
| chr19:38253104 | 0.957418 | 67.5438596 | 0.80769231 | 1 |
| chr1:204921255 | 0.957317 | 63.9423077 | 0.85365854 | 1 |
| chr1:224184849 | 0.957317 | 86.2263185 | 0.90243902 | 1 |
| chr11:48025532 | 0.957317 | 38.6540354 | 0.87804878 | 1 |
| chr5:16538197 | 0.957317 | 58.5883313 | 0.90243902 | 1 |
| chr1:153536113 | 0.957207 | 19.2156863 | 0.89189189 | 1 |
| chr1:153536123 | 0.957207 | 20 | 0.86486486 | 1 |
| chr9:124820965 | 0.957023 | 46.4102564 | 0.8490566 | 1 |
| chr21:41639350 | 0.956944 | 86.1642743 | 0.8 | 1 |
| chr9:113901422 | 0.956897 | 69.924812 | 0.89655172 | 0.94444444 |
| chr1:11965505 | 0.956731 | 67.9487179 | 0.87179487 | 1 |
| chr6:168638645 | 0.956446 | 84.5238095 | 0.87804878 | 1 |
| chr17:29582881 | 0.95625 | 92.1538462 | 0.85 | 1 |
| chr3:71074388 | 0.95625 | 97.2222222 | 0.8 | 1 |
| chr9:134486435 | 0.956044 | 93.0952381 | 0.8974359 | 1 |
| chr19:48350605 | 0.956044 | 38.1944444 | 0.97435897 | 0.85714286 |
| chr19:3656178 | 0.955997 | 68.967033 | 0.86885246 | 0.94736842 |
| chr11:118901571 | 0.955966 | 50.5555556 | 0.93181818 | 0.875 |
| chr1:43550383 | 0.955882 | 89.4444444 | 0.94117647 | 1 |
| chr12:123253763 | 0.955793 | 83.8068182 | 0.87804878 | 1 |
| chr16:30030208 | 0.955793 | 16.1764706 | 0.85365854 | 1 |
| chr21:41648785 | 0.955793 | 79.1025641 | 0.80487805 | 1 |
| chr9:91810447 | 0.955744 | 67.8466077 | 0.79661017 | 1 |
| chr10:79313956 | 0.955592 | 63.0409357 | 0.97368421 | 0.875 |
| chr6:169190341 | 0.955592 | 51.0683761 | 0.86842105 | 1 |
| chr16:85436880 | 0.955182 | 16.9047619 | 0.94117647 | 0.85714286 |
| chr1:1921165 | 0.955128 | 60.7142857 | 0.92307692 | 0.875 |
| chr1:205447760 | 0.955 | 67.2619048 | 0.925 | 1 |
| chr1:153536136 | 0.954955 | 8.89328063 | 0.89189189 | 1 |
| chr1:153536146 | 0.954955 | 24.047619 | 0.86486486 | 1 |
| chr11:19730746 | 0.954688 | 51.8965517 | 0.975 | 0.875 |
| chr3:141093639 | 0.954688 | 77.8954802 | 0.875 | 1 |
| chr2:159228539 | 0.954545 | 83.2167832 | 0.87755102 | 1 |
| chr21:17508899 | 0.954392 | 68.3333333 | 0.91891892 | 0.875 |
| chr11:66866541 | 0.954268 | 29.122807 | 0.92682927 | 1 |
| chr17:66830069 | 0.954268 | 10.1010101 | 0.85365854 | 1 |
| chr20:49345770 | 0.954268 | 14.7244806 | 0.87804878 | 1 |
| chr20:63835571 | 0.954268 | 16.4241486 | 0.87804878 | 1 |
| chr9:127200650 | 0.954268 | 84.9306431 | 0.85365854 | 1 |
| chr19:4055918 | 0.954082 | 85.3571429 | 0.9047619 | 1 |
| chr9:131810494 | 0.954023 | 47.6842105 | 0.84482759 | 1 |
| chr19:38253123 | 0.953984 | 39.5833333 | 0.96153846 | 0.85714286 |
| chr1:60903781 | 0.953832 | 32.7387802 | 0.9122807 | 0.94736842 |
| chr22:20014786 | 0.953782 | 70.8712121 | 0.80357143 | 1 |
| chr1:25771857 | 0.953721 | 49.5967742 | 0.9137931 | 0.94736842 |
| chr17:67652635 | 0.953408 | 78.8433908 | 0.95081967 | 0.89473684 |
| chr9:93166951 | 0.953333 | 45.2685422 | 0.98 | 0.86666667 |
| chr13:73218843 | 0.953125 | 78.6290323 | 0.825 | 1 |
| chr7:136045406 | 0.952744 | 31.0990338 | 0.97560976 | 0.875 |
| chr14:73228468 | 0.952721 | 40.9803922 | 0.88135593 | 1 |
| chr17:50831246 | 0.952721 | 57.1777547 | 0.88135593 | 0.94736842 |
| chr7:30171980 | 0.952641 | 89.2857143 | 0.86885246 | 0.94444444 |
| chr17:67652633 | 0.952545 | 84.841954 | 0.98360656 | 0.89473684 |
| chr1:28869065 | 0.952448 | 45.5882353 | 0.86440678 | 1 |
| chr13:113818528 | 0.952381 | 75.5744256 | 0.83333333 | 1 |
| chr2:200087031 | 0.952381 | 50 | 0.91666667 | 1 |
| chr2:200087051 | 0.952381 | 50 | 0.91666667 | 1 |
| chr6:168517547 | 0.952381 | 92.476489 | 0.81632653 | 1 |
| chr6:35534674 | 0.952326 | 18.3333333 | 0.86046512 | 1 |
| chr6:35534714 | 0.952326 | 27.8846154 | 0.90697674 | 1 |
| chr6:148509713 | 0.952035 | 14.2992424 | 0.81395349 | 1 |
| chr19:3656386 | 0.951977 | 80.8551308 | 0.96610169 | 0.88888889 |
| chr11:19832444 | 0.951961 | 52.7777778 | 0.95 | 0.82352941 |
| chr13:112775959 | 0.951754 | 85.5147059 | 0.92105263 | 1 |
| chr22:19632813 | 0.951705 | 68.8194444 | 0.84090909 | 1 |
| chr5:760546 | 0.951705 | 45.9961998 | 0.93181818 | 0.875 |
| chr19:3950318 | 0.951681 | 30.3846154 | 0.88235294 | 1 |
| chr1:60903756 | 0.951524 | 25.6363636 | 0.92982456 | 0.94736842 |
| chr7:8070969 | 0.951302 | 94.8684211 | 0.90163934 | 0.88235294 |
| chr16:70730157 | 0.951267 | 71.4912281 | 0.92982456 | 0.88888889 |
| chr20:49345761 | 0.95122 | 26.8518519 | 0.90243902 | 1 |
| chr3:45819148 | 0.95122 | 86.1904762 | 0.85365854 | 1 |
| chr6:19719834 | 0.95122 | 7.35632184 | 0.97560976 | 0.875 |
| chr6:34128254 | 0.95122 | 62.962963 | 0.97560976 | 0.875 |
| chr6:34128257 | 0.95122 | 90.2777778 | 0.90243902 | 0.875 |
| chr6:168130634 | 0.95122 | 75.990566 | 0.82926829 | 1 |
| chr1:15723893 | 0.951128 | 89.5833333 | 0.81578947 | 1 |
| chr16:85287162 | 0.950998 | 19.2265481 | 0.93103448 | 0.84210526 |
| chr7:1815269 | 0.95098 | 27.7777778 | 0.85294118 | 1 |
| chr9:93166950 | 0.950893 | 39.2592593 | 0.97619048 | 0.875 |
| chr16:85287116 | 0.950877 | 23.0424528 | 0.9 | 0.94736842 |
| chr17:67652568 | 0.95082 | 78.0357143 | 0.93442623 | 0.88888889 |
| chr12:123253794 | 0.95068 | 66.5789474 | 0.9047619 | 1 |
| chr16:85287098 | 0.95 | 34.3918011 | 0.85 | 1 |
| chr18:77077978 | 0.95 | 89.6808511 | 0.8 | 1 |
| chr19:46714207 | 0.95 | 6.11111111 | 0.82857143 | 1 |
| chr3:53702765 | 0.95 | 48.0769231 | 0.975 | 0.85714286 |
| chr7:3998211 | 0.95 | 52.2727273 | 0.85 | 1 |
| chr9:92729620 | 0.95 | 30.625 | 0.8 | 1 |
| chr16:85436709 | 0.949957 | 33.5486405 | 0.8852459 | 0.89473684 |
| chr11:66866538 | 0.949695 | 67.7083333 | 0.82926829 | 1 |
| chr22:44248796 | 0.949695 | 80.0986842 | 0.97560976 | 0.875 |
| chr6:34545471 | 0.949405 | 45.8333333 | 0.9047619 | 1 |
| chr7:102424320 | 0.949187 | 54.1666667 | 0.97560976 | 0.83333333 |
| chr17:602149 | 0.949153 | 78.1889764 | 0.96610169 | 0.8125 |
| chr20:22557482 | 0.949128 | 82.3076923 | 0.8372093 | 1 |
| chr6:3444679 | 0.94898 | 73.6231884 | 0.9047619 | 1 |
| chr17:57707866 | 0.94883 | 57.7777778 | 0.89473684 | 0.88888889 |
| chr16:11654442 | 0.948803 | 54.5454545 | 0.85106383 | 0.9375 |
| chr19:3655812 | 0.948765 | 30.3846154 | 0.98148148 | 0.86666667 |
| chr13:109334658 | 0.948718 | 22.1774194 | 0.87179487 | 1 |
| chr1:43550373 | 0.948529 | 88.75 | 0.91176471 | 1 |
| chr2:73080399 | 0.948413 | 89.8989899 | 0.8 | 1 |
| chr9:131810462 | 0.948343 | 75.6188119 | 0.9122807 | 0.94444444 |
| chr1:227920141 | 0.94832 | 40.6593407 | 1 | 0.77777778 |
| chr1:153536121 | 0.948198 | 10.8333333 | 0.89189189 | 1 |
| chr20:50035301 | 0.948171 | 88.7067395 | 0.80487805 | 1 |
| chr20:38700627 | 0.948171 | 3.01036683 | 0.95121951 | 0.875 |
| chr21:41553133 | 0.948171 | 70.2256245 | 0.85365854 | 1 |
| chr21:41648778 | 0.948171 | 66.1202186 | 0.82926829 | 1 |
| chr6:168130595 | 0.948171 | 76.3888889 | 0.82926829 | 1 |
| chr6:168539582 | 0.948122 | 64.4144144 | 0.95348837 | 0.92307692 |
| chr7:5276649 | 0.947991 | 39.514652 | 0.78723404 | 1 |
| chr21:40144935 | 0.947917 | 88.3484163 | 0.88095238 | 1 |
| chr19:3656261 | 0.947847 | 62.8289474 | 0.89090909 | 0.94736842 |
| chr3:139718818 | 0.947727 | 44.9494949 | 0.88636364 | 0.93333333 |
| chr1:10625828 | 0.947674 | 89.4444444 | 0.81395349 | 1 |
| chr3:185397835 | 0.947368 | 80.1282051 | 0.78947368 | 1 |
| chr7:110084403 | 0.947368 | 77.3504274 | 0.81578947 | 1 |
| chr5:176843791 | 0.947154 | 46.6403162 | 0.90243902 | 0.88888889 |
| chr6:40349351 | 0.94709 | 21.5384615 | 0.88095238 | 1 |
| chr9:113901566 | 0.94697 | 78.8781275 | 0.89090909 | 0.88888889 |
| chr21:41179996 | 0.946875 | 83.1300813 | 0.825 | 1 |
| chr4:528986 | 0.94666 | 71.657754 | 0.88135593 | 0.88235294 |
| chr12:48675786 | 0.946646 | 70.0892857 | 0.95121951 | 0.875 |
| chr19:13634667 | 0.946646 | 89.2720307 | 0.82926829 | 1 |
| chr6:168130574 | 0.946646 | 78.6764706 | 0.87804878 | 1 |
| chr19:3656190 | 0.946506 | 78.9181287 | 0.86885246 | 0.94736842 |
| chr1:202168439 | 0.946461 | 55.9382992 | 0.77586207 | 1 |
| chr12:124428932 | 0.946445 | 26.3988095 | 0.89473684 | 0.89473684 |
| chr6:34545461 | 0.946429 | 34.3137255 | 0.85714286 | 1 |
| chr7:3998240 | 0.946429 | 16.5178571 | 0.875 | 1 |
| chr3:127304410 | 0.945946 | 5.01253133 | 0.78378378 | 1 |
| chr16:89847327 | 0.945833 | 82.9326923 | 0.925 | 0.83333333 |
| chr1:226638169 | 0.945685 | 65.8181818 | 0.9047619 | 0.875 |
| chr1:181128695 | 0.945626 | 28.230042 | 0.85106383 | 1 |
| chr1:54356181 | 0.945578 | 79.6697626 | 0.78571429 | 1 |
| chr12:123253764 | 0.945578 | 85.4138133 | 0.85714286 | 1 |
| chr11:64056580 | 0.945513 | 86.0576923 | 0.88461538 | 0.91666667 |
| chr3:47236186 | 0.945437 | 65.4761905 | 0.85714286 | 0.94444444 |
| chr16:85436855 | 0.945378 | 17.4710425 | 0.90196078 | 0.85714286 |
| chr11:888597 | 0.945313 | 80.625 | 0.90384615 | 0.9375 |
| chr19:4010290 | 0.945313 | 15.6174334 | 0.9 | 1 |
| chr1:53320091 | 0.945122 | 61.1642743 | 1 | 0.75 |
| chr1:205257276 | 0.945122 | 61.5455665 | 0.7804878 | 1 |
| chr19:4010298 | 0.945122 | 19.1967325 | 0.87804878 | 1 |
| chr21:41648770 | 0.945122 | 87.7622378 | 0.92682927 | 1 |
| chr7:122306890 | 0.94494 | 44.3344892 | 0.9047619 | 1 |
| chr20:11271554 | 0.944907 | 27.2030651 | 0.86666667 | 0.94444444 |
| chr4:7911302 | 0.944643 | 44.8809524 | 0.95 | 0.85714286 |
| chr1:28869195 | 0.944444 | 10.0335149 | 0.93103448 | 0.94444444 |
| chr16:2455154 | 0.944444 | 89.2857143 | 0.86206897 | 1 |
| chr5:176843804 | 0.944444 | 59.1666667 | 0.7804878 | 1 |
| chr7:5276677 | 0.944444 | 57.7777778 | 0.87234043 | 0.88888889 |
| chr1:220653196 | 0.944251 | 54.2063492 | 0.97560976 | 0.85714286 |
| chr11:888641 | 0.944111 | 72.2506394 | 0.92307692 | 0.9375 |
| chr19:38253076 | 0.94391 | 44.8091382 | 0.90384615 | 0.91666667 |
| chr4:1858204 | 0.94391 | 70.0320513 | 0.97435897 | 0.75 |
| chr8:38495138 | 0.94391 | 32.2516026 | 0.94230769 | 0.88888889 |
| chr3:141093636 | 0.94375 | 78.4782609 | 0.85 | 1 |
| chr1:31627824 | 0.943598 | 43.6507937 | 0.85365854 | 1 |
| chr12:48675768 | 0.943598 | 86.6071429 | 0.95121951 | 0.875 |
| chr2:190034714 | 0.943598 | 92.1538462 | 0.82926829 | 1 |
| chr6:168638690 | 0.943598 | 65.1515152 | 0.87804878 | 1 |
| chr19:3656351 | 0.943503 | 33.3116883 | 0.96610169 | 0.83333333 |
| chr12:131864061 | 0.943434 | 48.6842105 | 0.83636364 | 1 |
| chr9:89591607 | 0.943414 | 58.0662651 | 0.91836735 | 0.90909091 |
| chr17:74823356 | 0.943277 | 78.5361842 | 0.94117647 | 0.92857143 |
| chr16:85287017 | 0.94303 | 27.9220779 | 0.98181818 | 0.8 |
| chr16:88887476 | 0.943027 | 28.7466844 | 0.95918367 | 0.91666667 |
| chr2:96174325 | 0.942982 | 33.5164835 | 0.89473684 | 1 |
| chr6:3444622 | 0.942935 | 52.2727273 | 0.91304348 | 0.9375 |
| chr1:5917479 | 0.942929 | 62.2580645 | 0.90909091 | 0.88888889 |
| chr1:1240684 | 0.942857 | 29.8148148 | 0.88571429 | 1 |
| chr12:130483799 | 0.942857 | 77.5 | 0.95 | 0.85714286 |
| chr13:110123705 | 0.942857 | 30.4377283 | 0.875 | 1 |
| chr6:168496991 | 0.942857 | 65.6862745 | 0.875 | 1 |
| chr8:53733503 | 0.942593 | 34.7486704 | 0.91666667 | 0.88888889 |
| chr7:105689129 | 0.942544 | 71.0606061 | 0.95 | 0.84210526 |
| chr2:113974997 | 0.942455 | 16.025641 | 0.97826087 | 0.82352941 |
| chr12:591924 | 0.942308 | 8.22072072 | 0.92307692 | 0.875 |
| chr15:65333873 | 0.942308 | 54.1666667 | 0.97435897 | 0.875 |
| chr3:194305584 | 0.942105 | 81.5636201 | 0.9 | 0.89473684 |
| chr19:4059381 | 0.942105 | 57.0082285 | 0.93333333 | 0.89473684 |
| chr18:59049874 | 0.942073 | 6.27185315 | 0.85365854 | 1 |
| chr1:224184822 | 0.942073 | 86.9747899 | 0.82926829 | 1 |
| chr11:76175216 | 0.942073 | 82.9090909 | 0.95121951 | 0.875 |
| chr6:168130594 | 0.942073 | 81.3043478 | 0.87804878 | 1 |
| chr19:3655954 | 0.941964 | 19.4444444 | 0.85416667 | 1 |
| chr19:34999970 | 0.941667 | 9.33908046 | 0.9 | 1 |
| chr6:26614205 | 0.941667 | 26.1363636 | 0.9 | 1 |
| chr10:78139388 | 0.941558 | 16.0106383 | 0.96969697 | 0.78571429 |
| chr11:122984321 | 0.941463 | 0.57142857 | 0.82926829 | 1 |
| chr19:3656172 | 0.941329 | 52.8532525 | 0.90163934 | 0.89473684 |
| chr1:43550344 | 0.941176 | 35 | 0.88235294 | 1 |
| chr12:129694731 | 0.941176 | 37.5 | 0.88235294 | 1 |
| chr19:3655828 | 0.941176 | 75.5555556 | 0.90909091 | 0.94117647 |
| chr7:151045548 | 0.941176 | 17.9487179 | 1 | 0.83333333 |
| chr12:130483815 | 0.941071 | 89.7368421 | 0.8 | 1 |
| chr5:78519838 | 0.941057 | 83.7719298 | 0.7804878 | 1 |
| chr11:888359 | 0.940897 | 55.1101073 | 0.95081967 | 0.84210526 |
| chr1:205032318 | 0.940776 | 34.4344609 | 0.9245283 | 0.94444444 |
| chr9:120242579 | 0.940625 | 65.1515152 | 0.89583333 | 0.9 |
| chr1:16486605 | 0.940549 | 87.5 | 0.90243902 | 0.875 |
| chr1:20348597 | 0.940549 | 56.8206522 | 0.90243902 | 1 |
| chr2:43203382 | 0.940549 | 81.6666667 | 0.95121951 | 0.875 |
| chr6:168130603 | 0.940549 | 84.0705128 | 0.90243902 | 0.875 |
| chr17:3695871 | 0.940541 | 16.25 | 0.89189189 | 1 |
| chr11:19714522 | 0.940476 | 0.75757576 | 0.88095238 | 1 |
| chr16:85439179 | 0.940476 | 81.5340909 | 0.94117647 | 0.92857143 |
| chr7:122306891 | 0.940476 | 45.7391304 | 0.88095238 | 1 |
| chr9:130233164 | 0.940407 | 69.047619 | 0.88372093 | 0.875 |
| chr2:47020392 | 0.940404 | 81.0790274 | 0.83636364 | 0.94444444 |
| chr19:3655938 | 0.940199 | 28.4271284 | 0.79069767 | 1 |
| chr8:58989422 | 0.94 | 94.6540881 | 0.825 | 1 |
| chr17:80001489 | 0.939815 | 85.9782609 | 0.91666667 | 0.83333333 |
| chr2:5530198 | 0.939815 | 35.5567227 | 0.85 | 1 |
| chr19:3656393 | 0.939736 | 91.2518854 | 0.89830508 | 0.94444444 |
| chr7:1815299 | 0.939542 | 22.875817 | 0.85294118 | 1 |
| chr21:43860194 | 0.939353 | 48.7903226 | 0.94339623 | 0.85714286 |
| chr6:168355227 | 0.939333 | 79.4736842 | 0.88 | 0.93333333 |
| chr1:20348596 | 0.939286 | 64.9122807 | 0.875 | 1 |
| chr7:5478011 | 0.93913 | 64.4137931 | 0.82608696 | 1 |
| chr2:200087040 | 0.939103 | 45.0067476 | 0.87179487 | 1 |
| chr19:48371166 | 0.939084 | 86.1620235 | 0.92982456 | 0.88888889 |
| chr16:22288921 | 0.939063 | 80.6134529 | 0.75 | 1 |
| chr19:12953833 | 0.939063 | 3.44064386 | 0.9 | 1 |
| chr11:888360 | 0.939024 | 78.4090909 | 0.92682927 | 1 |
| chr5:16538196 | 0.939024 | 71.8939394 | 0.7804878 | 1 |
| chr1:9251757 | 0.939024 | 39.5743146 | 0.90243902 | 1 |
| chr11:924646 | 0.939024 | 50.4716981 | 0.95121951 | 0.875 |
| chr12:131864094 | 0.939024 | 64.9122807 | 0.82926829 | 1 |
| chr2:25701937 | 0.939024 | 67.4107143 | 0.7804878 | 1 |
| chr6:168538915 | 0.939024 | 57.7380952 | 0.85365854 | 1 |
| chr9:127636823 | 0.939024 | 92.010582 | 0.75609756 | 1 |
| chr1:22809032 | 0.938953 | 42.2619048 | 0.86046512 | 1 |
| chr17:29065994 | 0.938802 | 85.1449275 | 0.83333333 | 1 |
| chr6:168517537 | 0.938776 | 94.7169059 | 0.7755102 | 1 |
| chr4:6308178 | 0.938641 | 36.3697706 | 0.89655172 | 0.94117647 |
| chr1:61198659 | 0.938519 | 82.9710145 | 0.93333333 | 0.93333333 |
| chr20:11271453 | 0.938448 | 69.2420682 | 0.79661017 | 0.94736842 |
| chr7:44151141 | 0.938194 | 54.4155844 | 0.8125 | 0.93333333 |
| chr19:18745096 | 0.938158 | 57.6973684 | 0.9 | 0.84210526 |
| chr7:30172111 | 0.937963 | 38.9285714 | 0.83333333 | 0.94444444 |
| chr6:34433316 | 0.937937 | 81.3852814 | 0.79545455 | 1 |
| chr12:119869919 | 0.9375 | 76.3888889 | 0.82926829 | 1 |
| chr13:110123752 | 0.9375 | 36.2899178 | 0.9 | 1 |
| chr17:29582888 | 0.9375 | 95.7935711 | 0.825 | 1 |
| chr18:36468445 | 0.9375 | 42.0168067 | 0.90243902 | 0.875 |
| chr19:4010297 | 0.9375 | 22.9813665 | 0.87804878 | 1 |
| chr19:39307494 | 0.9375 | 26.6590389 | 0.875 | 0.9 |
| chr20:63835620 | 0.9375 | 16.4224664 | 0.80487805 | 1 |
| chr21:41375919 | 0.9375 | 60.6060606 | 0.95121951 | 0.875 |
| chr5:6859175 | 0.9375 | 43.4210526 | 0.97560976 | 0.875 |
| chr6:37537680 | 0.9375 | 79.5726496 | 0.75609756 | 1 |
| chr7:100122236 | 0.9375 | 15.8852678 | 0.82926829 | 1 |
| chr9:93959419 | 0.9375 | 69.047619 | 0.91428571 | 0.875 |
| chr9:91810580 | 0.937382 | 77.8846154 | 0.89830508 | 0.94444444 |
| chr17:73726103 | 0.937281 | 54.4117647 | 0.91666667 | 0.94736842 |
| chr1:226589216 | 0.937276 | 81.6666667 | 0.74193548 | 1 |
| chr9:23452269 | 0.937255 | 52.1200144 | 0.86666667 | 0.88235294 |
| chr6:42963307 | 0.937158 | 61.8055556 | 0.91803279 | 0.88888889 |
| chr8:140321186 | 0.936911 | 91.5341812 | 0.86440678 | 0.94444444 |
| chr16:85287074 | 0.936842 | 34.5788043 | 0.8 | 1 |

TABLE 29
DNA methylation sites associated with SCLC-P

| MethylationSite | AUC | threshold | specificity | sensitivity |
|---|---|---|---|---|
| chr1:11032833 | 1 | 27.8846154 | 1 | 1 |
| chr1:15207848 | 1 | 8.33333333 | 1 | 1 |
| chr1:15945017 | 1 | 86.0576923 | 1 | 1 |
| chr1:17026179 | 1 | 81.6666667 | 1 | 1 |
| chr1:25741499 | 1 | 76.6666667 | 1 | 1 |
| chr1:27551002 | 1 | 68.4848485 | 1 | 1 |
| chr1:32327585 | 1 | 70.8333333 | 1 | 1 |
| chr1:42682988 | 1 | 3.03571429 | 1 | 1 |
| chr1:52365896 | 1 | 10.1010101 | 1 | 1 |
| chr1:56552275 | 1 | 53.9393939 | 1 | 1 |
| chr1:116407596 | 1 | 79.3706294 | 1 | 1 |
| chr1:147829076 | 1 | 96.7204797 | 1 | 1 |
| chr1:156538486 | 1 | 80.9615385 | 1 | 1 |
| chr1:156750879 | 1 | 5.60606061 | 1 | 1 |
| chr1:164576909 | 1 | 65.6160823 | 1 | 1 |
| chr1:197915932 | 1 | 61.3636364 | 1 | 1 |
| chr1:204289083 | 1 | 5 | 1 | 1 |
| chr1:204571233 | 1 | 66.6666667 | 1 | 1 |
| chr1:212618744 | 1 | 28.5714286 | 1 | 1 |
| chr1:212618752 | 1 | 15.7142857 | 1 | 1 |
| chr1:212638013 | 1 | 42.032967 | 1 | 1 |
| chr1:231162869 | 1 | 21.031746 | 1 | 1 |
| chr1:231162872 | 1 | 20.3216374 | 1 | 1 |
| chr1:231162880 | 1 | 29.9707602 | 1 | 1 |
| chr10:14365352 | 1 | 18.8995215 | 1 | 1 |
| chr10:55631088 | 1 | 95 | 1 | 1 |
| chr10:55631097 | 1 | 50.4444444 | 1 | 1 |
| chr10:97713353 | 1 | 54.1666667 | 1 | 1 |
| chr10:97713362 | 1 | 80.5555556 | 1 | 1 |
| chr10:123017613 | 1 | 11.7816092 | 1 | 1 |
| chr11:63997490 | 1 | 77.0833333 | 1 | 1 |
| chr11:65536690 | 1 | 78.4615385 | 1 | 1 |
| chr11:75410397 | 1 | 71.3636364 | 1 | 1 |
| chr11:77750252 | 1 | 27.1428571 | 1 | 1 |
| chr11:126313324 | 1 | 41.375969 | 1 | 1 |
| chr12:433809 | 1 | 61.525974 | 1 | 1 |
| chr12:7291128 | 1 | 53.6375661 | 1 | 1 |
| chr12:57128650 | 1 | 10.2678571 | 1 | 1 |
| chr12:67389978 | 1 | 65.1515152 | 1 | 1 |
| chr12:75861004 | 1 | 84.6590909 | 1 | 1 |
| chr12:81759542 | 1 | 73.8888889 | 1 | 1 |
| chr12:85489437 | 1 | 66.6666667 | 1 | 1 |
| chr12:101309330 | 1 | 70.0680272 | 1 | 1 |
| chr12:103993054 | 1 | 78.3625731 | 1 | 1 |
| chr12:104599362 | 1 | 86.1111111 | 1 | 1 |
| chr12:104599370 | 1 | 84.1666667 | 1 | 1 |
| chr12:105977083 | 1 | 47.7777778 | 1 | 1 |
| chr12:110245057 | 1 | 69.3549601 | 1 | 1 |
| chr12:110245058 | 1 | 70.7064793 | 1 | 1 |
| chr12:110245093 | 1 | 84.8154802 | 1 | 1 |
| chr12:111841988 | 1 | 6.79824561 | 1 | 1 |
| chr12:120456258 | 1 | 50 | 1 | 1 |
| chr12:122032753 | 1 | 33.8135593 | 1 | 1 |
| chr12:122032775 | 1 | 32.3584906 | 1 | 1 |
| chr12:124497504 | 1 | 20.7142857 | 1 | 1 |
| chr12:124524949 | 1 | 53.2467532 | 1 | 1 |
| chr12:124631301 | 1 | 76.7857143 | 1 | 1 |
| chr13:20345144 | 1 | 98.828125 | 1 | 1 |
| chr14:89701670 | 1 | 6.9047619 | 1 | 1 |
| chr14:105392906 | 1 | 80.3571429 | 1 | 1 |
| chr14:105392911 | 1 | 77.0833333 | 1 | 1 |
| chr15:43746172 | 1 | 6.54761905 | 1 | 1 |
| chr15:58701617 | 1 | 15.3947368 | 1 | 1 |
| chr15:74789232 | 1 | 25.3205128 | 1 | 1 |
| chr15:74789276 | 1 | 11.025641 | 1 | 1 |
| chr15:74789281 | 1 | 12.0289855 | 1 | 1 |
| chr16:234790 | 1 | 6.06617647 | 1 | 1 |
| chr16:234826 | 1 | 6.06617647 | 1 | 1 |
| chr16:648840 | 1 | 75 | 1 | 1 |
| chr16:1498961 | 1 | 32.9062406 | 1 | 1 |
| chr16:1746192 | 1 | 84.9906191 | 1 | 1 |
| chr16:2075993 | 1 | 5.08474576 | 1 | 1 |
| chr16:2964040 | 1 | 49.1666667 | 1 | 1 |
| chr16:4767447 | 1 | 8.11688312 | 1 | 1 |
| chr16:8621572 | 1 | 11.25 | 1 | 1 |
| chr16:28193303 | 1 | 3.57142857 | 1 | 1 |
| chr16:28863649 | 1 | 5 | 1 | 1 |
| chr16:29906931 | 1 | 83.7662338 | 1 | 1 |
| chr16:30125736 | 1 | 67.1328671 | 1 | 1 |
| chr16:30973692 | 1 | 66.6666667 | 1 | 1 |
| chr16:66696829 | 1 | 6.06617647 | 1 | 1 |
| chr16:68341637 | 1 | 46.4285714 | 1 | 1 |
| chr16:85611879 | 1 | 9.72222222 | 1 | 1 |
| chr17:21665928 | 1 | 22.8947368 | 1 | 1 |
| chr17:29576080 | 1 | 55.4179567 | 1 | 1 |
| chr17:34252590 | 1 | 92.8571429 | 1 | 1 |
| chr17:43187274 | 1 | 74.2857143 | 1 | 1 |
| chr17:48932449 | 1 | 77.5 | 1 | 1 |
| chr17:49816143 | 1 | 3.84615385 | 1 | 1 |
| chr17:63383824 | 1 | 38.4615385 | 1 | 1 |
| chr17:63383830 | 1 | 36.8055556 | 1 | 1 |
| chr17:63739476 | 1 | 52.0833333 | 1 | 1 |
| chr17:64661931 | 1 | 12.6984127 | 1 | 1 |
| chr17:68205724 | 1 | 7.63157895 | 1 | 1 |
| chr17:72165644 | 1 | 7.69230769 | 1 | 1 |
| chr17:76420086 | 1 | 4.76190476 | 1 | 1 |
| chr17:77439476 | 1 | 6.25 | 1 | 1 |
| chr17:77439513 | 1 | 9.375 | 1 | 1 |
| chr17:81248481 | 1 | 58.7912088 | 1 | 1 |
| chr17:81248517 | 1 | 80.4615385 | 1 | 1 |
| chr18:5543758 | 1 | 46.8137255 | 1 | 1 |
| chr18:5543760 | 1 | 47.9166667 | 1 | 1 |
| chr18:5543767 | 1 | 39.7058824 | 1 | 1 |
| chr18:5543774 | 1 | 64.5833333 | 1 | 1 |
| chr18:14907472 | 1 | 85.4545455 | 1 | 1 |
| chr18:28175723 | 1 | 51.3888889 | 1 | 1 |
| chr18:28175730 | 1 | 62.5 | 1 | 1 |
| chr18:28175734 | 1 | 65.4121864 | 1 | 1 |
| chr18:28175743 | 1 | 59.8566308 | 1 | 1 |
| chr18:57574427 | 1 | 67.9251701 | 1 | 1 |
| chr19:807444 | 1 | 20.3007519 | 1 | 1 |
| chr19:811705 | 1 | 62.7403846 | 1 | 1 |
| chr19:991153 | 1 | 57.7586207 | 1 | 1 |
| chr19:991161 | 1 | 49.1666667 | 1 | 1 |
| chr19:1633524 | 1 | 22.5524476 | 1 | 1 |
| chr19:1993008 | 1 | 13.2575758 | 1 | 1 |
| chr19:2115209 | 1 | 78.4090909 | 1 | 1 |
| chr19:5618219 | 1 | 79.2717087 | 1 | 1 |
| chr19:11483864 | 1 | 5 | 1 | 1 |
| chr19:11483872 | 1 | 5 | 1 | 1 |
| chr19:11483877 | 1 | 5 | 1 | 1 |
| chr19:11483882 | 1 | 15 | 1 | 1 |
| chr19:11483887 | 1 | 16.3461538 | 1 | 1 |
| chr19:11483902 | 1 | 8.33333333 | 1 | 1 |
| chr19:11483907 | 1 | 10 | 1 | 1 |
| chr19:11483912 | 1 | 5.55555556 | 1 | 1 |
| chr19:14206288 | 1 | 25.3846154 | 1 | 1 |
| chr19:14559510 | 1 | 51.754386 | 1 | 1 |
| chr19:16628375 | 1 | 12.5 | 1 | 1 |
| chr19:17209718 | 1 | 31.9642857 | 1 | 1 |
| chr19:17209725 | 1 | 29.1071429 | 1 | 1 |
| chr19:17209733 | 1 | 12.8571429 | 1 | 1 |
| chr19:17209752 | 1 | 18.5714286 | 1 | 1 |
| chr19:18778224 | 1 | 1.13636364 | 1 | 1 |
| chr19:35721194 | 1 | 34.1666667 | 1 | 1 |
| chr19:35944911 | 1 | 74.9016522 | 1 | 1 |
| chr19:38907710 | 1 | 60.3896104 | 1 | 1 |
| chr19:42211188 | 1 | 81.6666667 | 1 | 1 |
| chr19:45712682 | 1 | 45.2380952 | 1 | 1 |
| chr19:49464557 | 1 | 76.9736842 | 1 | 1 |
| chr19:49881697 | 1 | 65.4605263 | 1 | 1 |
| chr19:55284281 | 1 | 5.57275542 | 1 | 1 |
| chr19:55615832 | 1 | 81.5340909 | 1 | 1 |
| chr2:9003857 | 1 | 6.79824561 | 1 | 1 |
| chr2:61166980 | 1 | 59.1666667 | 1 | 1 |
| chr2:89848744 | 1 | 50.5050505 | 1 | 1 |
| chr2:190440760 | 1 | 28.5714286 | 1 | 1 |
| chr2:200981406 | 1 | 11.4074074 | 1 | 1 |
| chr2:202250239 | 1 | 21.3315217 | 1 | 1 |
| chr2:209772183 | 1 | 19.7115385 | 1 | 1 |
| chr2:224690031 | 1 | 48.0392157 | 1 | 1 |
| chr20:1185694 | 1 | 6.69642857 | 1 | 1 |
| chr20:62122215 | 1 | 3.61290323 | 1 | 1 |
| chr20:62309811 | 1 | 81.9444444 | 1 | 1 |
| chr20:62328882 | 1 | 87.593985 | 1 | 1 |
| chr20:63667368 | 1 | 76.3888889 | 1 | 1 |
| chr21:5137123 | 1 | 83.7719298 | 1 | 1 |
| chr21:25735328 | 1 | 13.3333333 | 1 | 1 |
| chr21:41879374 | 1 | 7.41758242 | 1 | 1 |
| chr21:43882191 | 1 | 30.5555556 | 1 | 1 |
| chr21:45277832 | 1 | 65.2941176 | 1 | 1 |
| chr22:31098838 | 1 | 64.375 | 1 | 1 |
| chr22:33058579 | 1 | 77.5 | 1 | 1 |
| chr22:40026246 | 1 | 73.8636364 | 1 | 1 |
| chr22:49884754 | 1 | 69.7777778 | 1 | 1 |
| chr3:13366052 | 1 | 73.3276158 | 1 | 1 |
| chr3:112523580 | 1 | 9.52380952 | 1 | 1 |
| chr3:112523614 | 1 | 29.1666667 | 1 | 1 |
| chr3:128428346 | 1 | 10.4761905 | 1 | 1 |
| chr3:138798882 | 1 | 86.8534483 | 1 | 1 |
| chr3:150408978 | 1 | 5.26675787 | 1 | 1 |
| chr3:157067444 | 1 | 2.60869565 | 1 | 1 |
| chr3:165348114 | 1 | 18.3333333 | 1 | 1 |
| chr3:165348116 | 1 | 18.3333333 | 1 | 1 |
| chr3:186783828 | 1 | 11.9505495 | 1 | 1 |
| chr3:197297920 | 1 | 5.04807692 | 1 | 1 |
| chr4:1229607 | 1 | 83.3333333 | 1 | 1 |
| chr4:4438102 | 1 | 57.0454545 | 1 | 1 |
| chr5:3750940 | 1 | 99.6296296 | 1 | 1 |
| chr5:6615113 | 1 | 79.5138889 | 1 | 1 |
| chr5:16179849 | 1 | 80 | 1 | 1 |
| chr5:16179857 | 1 | 55.7142857 | 1 | 1 |
| chr5:16179865 | 1 | 62.8571429 | 1 | 1 |
| chr5:16179878 | 1 | 80 | 1 | 1 |
| chr5:16179881 | 1 | 85 | 1 | 1 |
| chr5:16179883 | 1 | 95 | 1 | 1 |
| chr5:16180008 | 1 | 84.6153846 | 1 | 1 |
| chr5:178204129 | 1 | 30.6100218 | 1 | 1 |
| chr5:178204131 | 1 | 20.0980392 | 1 | 1 |
| chr6:3577167 | 1 | 21.8864469 | 1 | 1 |
| chr6:27279630 | 1 | 95.8333333 | 1 | 1 |
| chr6:73310426 | 1 | 77.8571429 | 1 | 1 |
| chr6:73310429 | 1 | 80 | 1 | 1 |
| chr6:73310454 | 1 | 54.4642857 | 1 | 1 |
| chr6:73310463 | 1 | 83.3333333 | 1 | 1 |
| chr6:125791186 | 1 | 10.7142857 | 1 | 1 |
| chr6:136290429 | 1 | 10.4761905 | 1 | 1 |
| chr7:996193 | 1 | 14.6428571 | 1 | 1 |
| chr7:1551166 | 1 | 87.4509804 | 1 | 1 |
| chr7:1849861 | 1 | 59.0277778 | 1 | 1 |
| chr7:1854236 | 1 | 14.8351648 | 1 | 1 |
| chr7:4730195 | 1 | 27.5641026 | 1 | 1 |
| chr7:5070131 | 1 | 79.1666667 | 1 | 1 |
| chr7:6333296 | 1 | 43.1818182 | 1 | 1 |
| chr7:15848453 | 1 | 22.2222222 | 1 | 1 |
| chr7:26247657 | 1 | 31.3131313 | 1 | 1 |
| chr7:29643153 | 1 | 78.8888889 | 1 | 1 |
| chr7:30043812 | 1 | 84.5238095 | 1 | 1 |
| chr7:43877706 | 1 | 80.1948052 | 1 | 1 |
| chr7:44146254 | 1 | 10.7142857 | 1 | 1 |
| chr7:44748739 | 1 | 8.88888889 | 1 | 1 |
| chr7:149022546 | 1 | 70.8333333 | 1 | 1 |
| chr8:31640044 | 1 | 15.2631579 | 1 | 1 |
| chr8:31640053 | 1 | 16.2962963 | 1 | 1 |
| chr8:31640067 | 1 | 17.3219373 | 1 | 1 |
| chr8:42842942 | 1 | 8.05555556 | 1 | 1 |
| chr8:80578027 | 1 | 9.54545455 | 1 | 1 |
| chr9:107245787 | 1 | 71.25 | 1 | 1 |
| chr9:120913537 | 1 | 62.5 | 1 | 1 |
| chr9:126408197 | 1 | 86.5100976 | 1 | 1 |
| chr9:131584010 | 1 | 75.877193 | 1 | 1 |
| chr9:136197462 | 1 | 3.125 | 1 | 1 |
| chr9:136451036 | 1 | 63.8586957 | 1 | 1 |
| chr9:136451222 | 1 | 64.2857143 | 1 | 1 |
| chr9:136451519 | 1 | 71.6008772 | 1 | 1 |
| chr9:137013461 | 1 | 18.5185185 | 1 | 1 |
| chr9:137013470 | 1 | 28.5714286 | 1 | 1 |
| chr9:137013473 | 1 | 60.7142857 | 1 | 1 |
| chr9:137013486 | 1 | 43.3982684 | 1 | 1 |
| chr9:137278360 | 1 | 43.75 | 1 | 1 |
| chr9:137278384 | 1 | 68.3333333 | 1 | 1 |
| chr9:137278402 | 1 | 43.75 | 1 | 1 |
| chr9:137278488 | 1 | 13.2716049 | 1 | 1 |
| chrX:150899022 | 1 | 65.7738095 | 1 | 1 |
| chr6:34553001 | 0.996711 | 29.4047619 | 0.98684211 | 1 |
| chr12:118980854 | 0.996667 | 11.7592593 | 0.98666667 | 1 |
| chr19:811773 | 0.996667 | 60.9090909 | 0.98666667 | 1 |
| chr2:130345606 | 0.996622 | 79.9342105 | 0.98648649 | 1 |
| chr2:202250236 | 0.996377 | 17.5 | 0.98550725 | 1 |
| chr7:1005784 | 0.996377 | 87.3015873 | 0.98550725 | 1 |
| chr19:7934275 | 0.995763 | 50.6756757 | 0.98305085 | 1 |
| chr14:34024477 | 0.995726 | 38.0392157 | 0.97435897 | 1 |
| chr7:107390004 | 0.995614 | 69.8198198 | 0.98245614 | 1 |
| chr1:25216043 | 0.99537 | 41.6666667 | 0.98148148 | 1 |
| chr15:85610808 | 0.995169 | 82.5098039 | 0.98550725 | 1 |
| chr19:5668214 | 0.995 | 31.832265 | 0.98 | 1 |
| chr7:5297184 | 0.995 | 35.6504932 | 0.98 | 1 |
| chr1:241356405 | 0.994898 | 12.406015 | 0.97959184 | 1 |
| chr10:102714534 | 0.994898 | 3.22439567 | 0.97959184 | 1 |
| chr8:31640064 | 0.994872 | 16.1403509 | 0.98461538 | 1 |
| chr12:120967099 | 0.994792 | 58.3333333 | 0.984375 | 1 |
| chr19:1393965 | 0.994792 | 61.6082603 | 0.97916667 | 1 |
| chr15:25438570 | 0.994643 | 4.375 | 0.97142857 | 1 |
| chr15:74789270 | 0.994565 | 27.8861789 | 0.97826087 | 1 |
| chr7:5297124 | 0.994565 | 40.4304029 | 0.97826087 | 1 |
| chr8:123443315 | 0.994565 | 49.2068619 | 0.97826087 | 1 |
| chr1:180269892 | 0.994444 | 44.3069887 | 0.97777778 | 1 |
| chr19:1456332 | 0.994444 | 19.1919192 | 0.97777778 | 1 |
| chr16:73028748 | 0.994186 | 45.0657895 | 0.97674419 | 1 |
| chr2:75056446 | 0.994186 | 35.485348 | 0.97674419 | 1 |
| chr19:18543346 | 0.994152 | 0.64750789 | 0.98245614 | 1 |
| chr2:75056435 | 0.994048 | 36.6649755 | 0.97619048 | 1 |
| chr2:75056445 | 0.994048 | 45.625 | 0.97619048 | 1 |
| chr16:29876586 | 0.99375 | 83.7719298 | 0.975 | 1 |
| chr6:43771035 | 0.99359 | 4.46428571 | 0.97435897 | 1 |
| chr12:124344199 | 0.993421 | 68.6065574 | 0.98684211 | 1 |
| chr8:139649190 | 0.993333 | 65.6862745 | 0.98666667 | 1 |
| chr2:230068915 | 0.993243 | 93.75 | 0.98648649 | 1 |
| chr1:6461087 | 0.993151 | 72.1661055 | 0.97260274 | 1 |
| chr1:234661304 | 0.993151 | 68.3333333 | 0.98630137 | 1 |
| chr12:7824990 | 0.993151 | 56.5860215 | 0.98630137 | 1 |
| chr16:71931876 | 0.993151 | 80.2777778 | 0.97260274 | 1 |
| chr21:36736273 | 0.993151 | 98.3870968 | 0.98630137 | 1 |
| chr3:183636771 | 0.993151 | 8.71212121 | 0.98630137 | 1 |
| chr5:143405413 | 0.993151 | 10.1010101 | 0.98630137 | 1 |
| chr10:69402478 | 0.993056 | 92.1212121 | 0.97222222 | 1 |
| chr14:104887313 | 0.993056 | 72.3809524 | 0.98611111 | 1 |
| chr16:1315523 | 0.993056 | 85.7843137 | 0.97222222 | 1 |
| chr7:2295681 | 0.993056 | 79.1666667 | 0.97222222 | 1 |
| chr1:214108494 | 0.992857 | 55.7692308 | 0.98571429 | 1 |
| chr1:236064214 | 0.992857 | 96.4285714 | 0.98571429 | 1 |
| chr2:44168465 | 0.992857 | 10.960961 | 0.98571429 | 1 |
| chr2:114662163 | 0.992857 | 96.1538462 | 0.98571429 | 1 |
| chr3:107999730 | 0.992857 | 55.7692308 | 0.98571429 | 1 |
| chr6:3577155 | 0.992857 | 24.9762583 | 0.98571429 | 1 |
| chr6:3577177 | 0.992857 | 25.3407984 | 0.98571429 | 1 |
| chr7:57818168 | 0.992857 | 54.2053446 | 0.97142857 | 1 |
| chr1:41324219 | 0.992754 | 52.1008403 | 0.98550725 | 1 |
| chr1:213051051 | 0.992754 | 78.8888889 | 0.98550725 | 1 |
| chr11:4521815 | 0.992754 | 96.875 | 0.98550725 | 1 |
| chr12:43552603 | 0.992754 | 96.6666667 | 0.98550725 | 1 |
| chr17:35488134 | 0.992754 | 97.7272727 | 0.98550725 | 1 |
| chr3:14401986 | 0.992754 | 1.92307692 | 0.98550725 | 1 |
| chr6:13450944 | 0.992754 | 82.0855615 | 0.98550725 | 1 |
| chr9:101738883 | 0.992754 | 53.015873 | 0.98550725 | 1 |
| chr1:167221470 | 0.992647 | 3.125 | 0.97058824 | 1 |
| chr16:88880826 | 0.992647 | 2.63157895 | 0.98529412 | 1 |
| chr19:3652127 | 0.992647 | 83.974359 | 0.98529412 | 1 |
| chr20:17396399 | 0.992647 | 1.5625 | 0.98529412 | 1 |
| chr12:104599364 | 0.992537 | 84.8765432 | 0.98507463 | 1 |
| chr16:87827109 | 0.992537 | 4.54545455 | 0.98507463 | 1 |
| chr19:17873403 | 0.992537 | 0.76923077 | 0.98507463 | 1 |
| chr19:17873409 | 0.992537 | 19.4444444 | 0.98507463 | 1 |
| chr19:17873411 | 0.992537 | 8.33333333 | 0.98507463 | 1 |
| chr6:63656136 | 0.992537 | 75.2525253 | 0.98507463 | 1 |
| chr12:45216305 | 0.992424 | 5.45905707 | 0.98484848 | 1 |
| chr12:57837371 | 0.992424 | 77.3504274 | 0.98484848 | 1 |
| chr16:21557088 | 0.992424 | 95.2380952 | 0.98484848 | 1 |
| chr17:34331252 | 0.992424 | 95 | 0.98484848 | 1 |
| chr21:39054428 | 0.992424 | 20.8333333 | 0.98484848 | 1 |
| chr7:3910633 | 0.992424 | 2.94117647 | 0.98484848 | 1 |
| chr16:3655081 | 0.992308 | 4.28571429 | 0.98461538 | 1 |
| chr18:12324746 | 0.992308 | 82.3076923 | 0.98461538 | 1 |
| chr9:76701152 | 0.992308 | 51.9230769 | 0.98461538 | 1 |
| chr19:1648427 | 0.992248 | 66.1490683 | 0.97674419 | 1 |
| chr20:47468584 | 0.992188 | 8.33333333 | 0.984375 | 1 |
| chr3:96841981 | 0.992188 | 71.875 | 0.984375 | 1 |
| chr1:239386481 | 0.992063 | 41.0526316 | 0.98412698 | 1 |
| chr19:3088153 | 0.992063 | 31.541769 | 0.98412698 | 1 |
| chr20:17396185 | 0.992063 | 14.7058824 | 0.98412698 | 1 |
| chr6:27557116 | 0.992063 | 98.8372093 | 0.98412698 | 1 |
| chr6:55680614 | 0.992063 | 95 | 0.98412698 | 1 |
| chr7:5297111 | 0.992063 | 0.69444444 | 0.98412698 | 1 |
| chr19:58440360 | 0.991935 | 96.1538462 | 0.98387097 | 1 |
| chr7:151111578 | 0.991935 | 5.88235294 | 0.98387097 | 1 |
| chr8:139649329 | 0.991935 | 22.5 | 0.98387097 | 1 |
| chr19:28520192 | 0.991803 | 96.875 | 0.98360656 | 1 |
| chr6:34778112 | 0.991803 | 80.5555556 | 0.98360656 | 1 |
| chr9:131112163 | 0.991803 | 64.7163121 | 0.98360656 | 1 |
| chr7:102201787 | 0.991667 | 92.7291886 | 0.96 | 1 |
| chr8:42153025 | 0.991667 | 12.1428571 | 0.98333333 | 1 |
| chr8:42622755 | 0.991667 | 5 | 0.98333333 | 1 |
| chr1:154174789 | 0.991525 | 71.7948718 | 0.98305085 | 1 |
| chr17:49816136 | 0.991525 | 2.5 | 0.98305085 | 1 |
| chr19:7934266 | 0.991525 | 80.625 | 0.98305085 | 1 |
| chr19:13207507 | 0.991525 | 83.7116423 | 0.96610169 | 1 |
| chr2:17317571 | 0.991525 | 98.3870968 | 0.98305085 | 1 |
| chr5:3754702 | 0.991525 | 80.5555556 | 0.98305085 | 1 |
| chr6:24626028 | 0.991525 | 8.68486352 | 0.98305085 | 1 |
| chr6:44653805 | 0.991525 | 58.4848485 | 0.98305085 | 1 |
| chr2:173307231 | 0.991477 | 74.4166667 | 0.97727273 | 1 |
| chr6:22509101 | 0.991379 | 3.84615385 | 0.98275862 | 1 |
| chr8:107889762 | 0.991379 | 4.16666667 | 0.98275862 | 1 |
| chr5:128879466 | 0.991197 | 88.3333333 | 0.98591549 | 1 |
| chr19:3094373 | 0.991111 | 3.44466537 | 0.98666667 | 1 |
| chr17:72235698 | 0.991071 | 4.54545455 | 0.98214286 | 1 |
| chr21:34510358 | 0.991071 | 7.14285714 | 0.98214286 | 1 |
| chr9:137013468 | 0.990909 | 20.3007519 | 0.98181818 | 1 |
| chr9:137013480 | 0.990909 | 36.5079365 | 0.98181818 | 1 |
| chr1:220001751 | 0.990868 | 27.8361345 | 0.98630137 | 1 |
| chr5:308909 | 0.990868 | 76.7857143 | 0.97260274 | 1 |
| chr1:155544582 | 0.990741 | 79.4736842 | 0.98148148 | 1 |
| chr1:204089544 | 0.990741 | 58.5714286 | 0.97222222 | 1 |
| chr11:107970310 | 0.990741 | 49.95671 | 0.97222222 | 1 |
| chr12:95066688 | 0.990741 | 34.1346154 | 0.98148148 | 1 |
| chr19:16553992 | 0.990741 | 70.5802969 | 0.97222222 | 1 |
| chr5:16179995 | 0.990741 | 85.2941176 | 0.98148148 | 1 |
| chr5:16180003 | 0.990741 | 90.2777778 | 0.98148148 | 1 |
| chr5:25900907 | 0.990741 | 3.03030303 | 0.98148148 | 1 |
| chr8:35235230 | 0.990741 | 96.4285714 | 0.98148148 | 1 |
| chr16:28617820 | 0.990672 | 31.712963 | 0.98507463 | 1 |
| chr17:76419894 | 0.99061 | 18.8461538 | 0.98591549 | 1 |
| chr19:48194427 | 0.99061 | 20.0909091 | 0.98591549 | 1 |
| chr7:14695759 | 0.99061 | 33.0602637 | 0.97183099 | 1 |
| chr1:6470978 | 0.990566 | 63.1877023 | 0.98113208 | 1 |
| chr1:23636028 | 0.990566 | 73.2142857 | 0.98113208 | 1 |
| chr20:350196 | 0.990476 | 75.9090909 | 0.97142857 | 1 |
| chr1:31193833 | 0.990385 | 44.1558442 | 0.98076923 | 1 |
| chr8:126862783 | 0.990385 | 3.84615385 | 0.98076923 | 1 |
| chr11:78046094 | 0.990196 | 24.4047619 | 0.97058824 | 1 |
| chr12:132528585 | 0.990196 | 52.2727273 | 0.96078431 | 1 |
| chr18:79375872 | 0.990196 | 77.3182957 | 0.97058824 | 1 |
| chr19:652099 | 0.990196 | 78.8847118 | 0.96078431 | 1 |
| chr21:9873175 | 0.990196 | 47.3214286 | 0.98039216 | 1 |
| chr7:57821358 | 0.990196 | 68.0555556 | 0.98529412 | 1 |
| chr7:101967581 | 0.990196 | 66.0714286 | 0.98039216 | 1 |
| chr8:144107705 | 0.990196 | 26.3931889 | 0.98529412 | 1 |
| chr12:62602516 | 0.99005 | 6.11111111 | 0.97014925 | 1 |
| chr1:6460819 | 0.99 | 33.3854286 | 0.96 | 1 |
| chr1:244198034 | 0.99 | 41.6373239 | 0.96 | 1 |
| chr19:5704451 | 0.99 | 78.5691971 | 0.96 | 1 |
| chr9:137278356 | 0.99 | 21.6715332 | 0.98 | 1 |
| chr10:76887333 | 0.989865 | 65.3370787 | 0.95945946 | 1 |
| chr19:1433766 | 0.989865 | 72.0779221 | 0.95945946 | 1 |
| chr19:13830675 | 0.989865 | 77.6344086 | 0.98648649 | 1 |
| chr20:37985816 | 0.989865 | 61.25 | 0.95945946 | 1 |
| chr1:15945048 | 0.989796 | 85.1648352 | 0.97959184 | 1 |
| chr14:23078131 | 0.989796 | 73.8636364 | 0.97959184 | 1 |
| chr9:133459357 | 0.989796 | 11.6883117 | 0.95918367 | 1 |
| chr8:31640070 | 0.989744 | 19.0046296 | 0.98461538 | 1 |
| chr12:62799867 | 0.989726 | 41.3483931 | 0.98630137 | 1 |
| chr5:32446706 | 0.989726 | 36.0988896 | 0.98630137 | 1 |
| chr12:120967103 | 0.989583 | 70.2380952 | 0.96875 | 1 |
| chr5:128879583 | 0.989437 | 44.820296 | 0.95774648 | 1 |
| chr15:69087040 | 0.989418 | 34.5771144 | 0.96825397 | 1 |
| chr20:347269 | 0.989418 | 6.69642857 | 0.98412698 | 1 |
| chr9:132325224 | 0.989362 | 92.5824176 | 0.95744681 | 1 |
| chr16:78195948 | 0.989362 | 7.14285714 | 0.9787234 | 1 |
| chr19:28824412 | 0.989362 | 97.3684211 | 0.9787234 | 1 |
| chr19:44753544 | 0.989362 | 97.0588235 | 0.9787234 | 1 |
| chr22:11288465 | 0.989362 | 63.0681818 | 0.9787234 | 1 |
| chr17:72123931 | 0.989286 | 68.1885125 | 0.95714286 | 1 |
| chr17:72124220 | 0.989286 | 12.3161765 | 0.98571429 | 1 |
| chr17:72124238 | 0.989286 | 17.7871148 | 0.98571429 | 1 |
| chr2:36973823 | 0.98913 | 79.5138889 | 0.97826087 | 1 |
| chr5:16179886 | 0.98913 | 75 | 0.97826087 | 1 |
| chr20:44560834 | 0.988971 | 19.6428571 | 0.97058824 | 1 |
| chr12:50057840 | 0.988889 | 3.82414353 | 0.97777778 | 1 |
| chr19:1456317 | 0.988889 | 16.6666667 | 0.97777778 | 1 |
| chr7:1421100 | 0.988889 | 2.94117647 | 0.97777778 | 1 |
| chr8:123443370 | 0.988889 | 46.6494845 | 0.95555556 | 1 |
| chr17:82626176 | 0.988806 | 86.9334719 | 0.95522388 | 1 |
| chr18:63283742 | 0.988701 | 59.1269841 | 0.96610169 | 1 |
| chr11:65650576 | 0.988636 | 88.3965517 | 0.95454545 | 1 |
| chr12:31591065 | 0.988636 | 11.8055556 | 0.97727273 | 1 |
| chr16:73028716 | 0.988636 | 76.7857143 | 0.95454545 | 1 |
| chr6:106135145 | 0.988636 | 45.6547619 | 0.96969697 | 1 |
| chr1:230399219 | 0.988462 | 21.2053571 | 0.98461538 | 1 |
| chr17:80599748 | 0.988462 | 12.8264209 | 0.98461538 | 1 |
| chr3:136614019 | 0.988462 | 87.8571429 | 0.96923077 | 1 |
| chr9:93826472 | 0.988462 | 44.2307692 | 0.98461538 | 1 |
| chr19:812407 | 0.988372 | 70.8333333 | 0.97674419 | 1 |
| chr6:135771171 | 0.988372 | 92.0454545 | 0.97674419 | 1 |
| chr9:137278500 | 0.988304 | 45.2008929 | 0.98245614 | 1 |
| chr16:3987194 | 0.988281 | 83.5034014 | 0.96875 | 1 |
| chr17:49810078 | 0.988281 | 28.8797062 | 0.984375 | 1 |
| chr2:164717792 | 0.988095 | 86.6071429 | 0.97142857 | 1 |
| chr22:40600900 | 0.988095 | 84.2751843 | 0.97619048 | 1 |
| chr3:123976907 | 0.988095 | 56.4393939 | 0.97619048 | 1 |
| chr3:123976934 | 0.988095 | 81.953867 | 0.97619048 | 1 |
| chr2:89244905 | 0.988014 | 91 | 0.98630137 | 1 |
| chr19:5668194 | 0.987981 | 28.709893 | 0.96153846 | 1 |
| chr11:78046073 | 0.987923 | 20.8333333 | 0.95652174 | 1 |
| chr9:127507984 | 0.987923 | 56.8181818 | 0.95652174 | 1 |
| chr9:120877473 | 0.987805 | 7.48663102 | 0.97560976 | 1 |
| chr1:159210159 | 0.987705 | 90.2380952 | 0.98360656 | 1 |
| chr6:44441882 | 0.987705 | 14.1666667 | 0.98360656 | 1 |
| chr8:101950035 | 0.987705 | 89.9038462 | 0.98360656 | 1 |
| chr1:11331701 | 0.9875 | 51.4705882 | 0.96666667 | 1 |
| chr19:1992998 | 0.9875 | 26.1363636 | 0.975 | 1 |
| chr19:1993003 | 0.9875 | 23.3333333 | 0.975 | 1 |
| chr9:128704978 | 0.9875 | 67.816092 | 0.975 | 1 |
| chr9:124453638 | 0.987421 | 2.95745921 | 0.98113208 | 1 |
| chr19:1468243 | 0.987288 | 31.6666667 | 0.94915254 | 1 |
| chr16:2429260 | 0.987179 | 5.20833333 | 0.97435897 | 1 |
| chr20:62309824 | 0.987179 | 75.7352941 | 0.97435897 | 1 |
| chr7:44146237 | 0.987179 | 1.72413793 | 0.97435897 | 1 |
| chr8:31640050 | 0.987179 | 14.1750842 | 0.98461538 | 1 |
| chr9:94914976 | 0.987179 | 62.2222222 | 0.98461538 | 1 |
| chr12:37419025 | 0.987069 | 83.8888889 | 0.98275862 | 1 |
| chr12:45776142 | 0.986979 | 22.3296888 | 0.984375 | 1 |
| chr17:42670166 | 0.986842 | 78.125 | 0.97368421 | 1 |
| chr19:1633551 | 0.986842 | 25.625 | 0.97368421 | 1 |
| chr9:137281710 | 0.986842 | 70 | 0.97368421 | 1 |
| chr1:214281447 | 0.986842 | 5.15873016 | 0.98684211 | 1 |
| chr6:34553086 | 0.986842 | 68.7062937 | 0.96052632 | 1 |
| chr1:167918494 | 0.986742 | 64.1121495 | 0.98484848 | 1 |
| chr17:72123590 | 0.986667 | 2.08333333 | 0.97333333 | 1 |
| chr19:45067160 | 0.986667 | 84.8287238 | 0.98666667 | 1 |
| chr6:157300486 | 0.986667 | 78.9784946 | 0.97333333 | 1 |
| chr7:1734991 | 0.986667 | 2.17391304 | 0.97333333 | 1 |
| chr7:1734996 | 0.986667 | 2.17391304 | 0.97333333 | 1 |
| chr1:70338695 | 0.986607 | 82.3611111 | 0.96428571 | 1 |
| chr19:18868002 | 0.986607 | 55.6818182 | 0.94642857 | 1 |
| chr9:14041343 | 0.986607 | 86.1904762 | 0.96428571 | 1 |
| chr10:76887324 | 0.986486 | 71.657754 | 0.94594595 | 1 |
| chr19:7934351 | 0.986486 | 49.5961175 | 0.94594595 | 1 |
| chr19:13644740 | 0.986486 | 55.2083333 | 0.98648649 | 1 |
| chr2:5691280 | 0.986486 | 89.2857143 | 0.97297297 | 1 |
| chr5:134550960 | 0.986486 | 59.1025641 | 0.97297297 | 1 |
| chr12:95633568 | 0.986364 | 24.9482402 | 0.98181818 | 1 |
| chr1:6460956 | 0.986301 | 50.9671993 | 0.98630137 | 1 |
| chr16:17134583 | 0.986301 | 16.6666667 | 0.97260274 | 1 |
| chr16:17134595 | 0.986301 | 8.69565217 | 0.97260274 | 1 |
| chr16:61149835 | 0.986301 | 85.9375 | 0.97260274 | 1 |
| chr19:4101376 | 0.986301 | 92.0807453 | 0.97260274 | 1 |
| chr19:37692212 | 0.986301 | 97.3684211 | 0.97260274 | 1 |
| chr6:1494847 | 0.986301 | 99.7641509 | 0.97260274 | 1 |
| chr7:38178231 | 0.986301 | 4.2791762 | 0.97260274 | 1 |
| chr7:71132295 | 0.986301 | 98.5915493 | 0.97260274 | 1 |
| chr11:134072686 | 0.986111 | 59.6296296 | 0.95833333 | 1 |
| chr16:2720277 | 0.986111 | 1.02040816 | 0.97222222 | 1 |
| chr16:11345580 | 0.986111 | 6.9047619 | 0.95833333 | 1 |
| chr17:29613358 | 0.986111 | 87.7777778 | 0.95555556 | 1 |
| chr18:14867181 | 0.986111 | 20.1923077 | 0.97222222 | 1 |
| chr19:3613344 | 0.986111 | 91.1538462 | 0.97222222 | 1 |
| chr2:209772188 | 0.986111 | 23.9548495 | 0.98611111 | 1 |
| chr6:34552991 | 0.986111 | 75.9090909 | 0.97222222 | 1 |
| chr6:88962788 | 0.986111 | 93.3333333 | 0.97222222 | 1 |
| chr12:105977085 | 0.985915 | 16.3333333 | 0.98591549 | 1 |
| chr17:7479367 | 0.985915 | 4.62765957 | 0.97183099 | 1 |
| chr17:21379559 | 0.985915 | 79.9498747 | 0.95774648 | 1 |
| chr19:10502839 | 0.985915 | 4.63609172 | 0.98591549 | 1 |
| chr19:39178093 | 0.985915 | 74.3421053 | 0.98591549 | 1 |
| chr21:43313606 | 0.985915 | 59.9431818 | 0.97183099 | 1 |
| chr22:31306646 | 0.985915 | 5.88678433 | 0.97183099 | 1 |
| chr3:11717633 | 0.985915 | 50.0938967 | 0.97183099 | 1 |
| chr3:139539693 | 0.985915 | 15.4761905 | 0.98591549 | 1 |
| chr7:4730228 | 0.985915 | 59.0277778 | 0.97183099 | 1 |
| chr4:154375969 | 0.985849 | 90.1785714 | 0.98113208 | 1 |

TABLE 30
DNA methylation sites associated with SCLC-I

| MethylationSite | AUC | threshold | specificity | sensitivity |
|---|---|---|---|---|
| chr1:1040462 | 1 | 87.4509804 | 1 | 1 |
| chr1:1040475 | 1 | 90.3571429 | 1 | 1 |
| chr1:1682754 | 1 | 69.6153846 | 1 | 1 |
| chr1:6249894 | 1 | 56.8181818 | 1 | 1 |
| chr1:6249902 | 1 | 65.9090909 | 1 | 1 |
| chr1:6249914 | 1 | 66.8831169 | 1 | 1 |
| chr1:6249917 | 1 | 70.4545455 | 1 | 1 |
| chr1:12191832 | 1 | 79.7979798 | 1 | 1 |
| chr1:15741578 | 1 | 8.57142857 | 1 | 1 |
| chr1:16980570 | 1 | 94.4444444 | 1 | 1 |
| chr1:16980579 | 1 | 71.7948718 | 1 | 1 |
| chr1:16980593 | 1 | 94.4444444 | 1 | 1 |
| chr1:18630506 | 1 | 82.5757576 | 1 | 1 |
| chr1:20487144 | 1 | 10.7954545 | 1 | 1 |
| chr1:21574155 | 1 | 60.3896104 | 1 | 1 |
| chr1:23801501 | 1 | 77.8195489 | 1 | 1 |
| chr1:24730777 | 1 | 26.8965517 | 1 | 1 |
| chr1:24730787 | 1 | 60 | 1 | 1 |
| chr1:24730788 | 1 | 57.2139303 | 1 | 1 |
| chr1:24730796 | 1 | 44.1304348 | 1 | 1 |
| chr1:24730797 | 1 | 51.6583748 | 1 | 1 |
| chr1:25195867 | 1 | 87.1052632 | 1 | 1 |
| chr1:31065986 | 1 | 4.4834308 | 1 | 1 |
| chr1:36323440 | 1 | 8.07354117 | 1 | 1 |
| chr1:39491967 | 1 | 3.74331551 | 1 | 1 |
| chr1:43285575 | 1 | 49.2324561 | 1 | 1 |
| chr1:43285577 | 1 | 51.8640351 | 1 | 1 |
| chr1:43369421 | 1 | 87.3826903 | 1 | 1 |
| chr1:89843439 | 1 | 84.4444444 | 1 | 1 |
| chr1:89843473 | 1 | 92.2222222 | 1 | 1 |
| chr1:145960916 | 1 | 7.57142857 | 1 | 1 |
| chr1:153532886 | 1 | 83.1521739 | 1 | 1 |
| chr1:153532890 | 1 | 89.4927536 | 1 | 1 |
| chr1:156908050 | 1 | 97.5 | 1 | 1 |
| chr1:163404039 | 1 | 52.5252525 | 1 | 1 |
| chr1:163423088 | 1 | 63.125 | 1 | 1 |
| chr1:163423133 | 1 | 67.5 | 1 | 1 |
| chr1:163423188 | 1 | 77.540107 | 1 | 1 |
| chr1:163423194 | 1 | 60.2240896 | 1 | 1 |
| chr1:163423199 | 1 | 75.6493506 | 1 | 1 |
| chr1:163546298 | 1 | 70.1929366 | 1 | 1 |
| chr1:164136579 | 1 | 64.4409938 | 1 | 1 |
| chr1:164136587 | 1 | 73.5714286 | 1 | 1 |
| chr1:164136632 | 1 | 4.16666667 | 1 | 1 |
| chr1:164236230 | 1 | 62.2159091 | 1 | 1 |
| chr1:164236242 | 1 | 49.1477273 | 1 | 1 |
| chr1:165235591 | 1 | 5.75016523 | 1 | 1 |
| chr1:167796909 | 1 | 75 | 1 | 1 |
| chr1:208601823 | 1 | 67.7071006 | 1 | 1 |
| chr1:213771516 | 1 | 27.3026316 | 1 | 1 |
| chr1:213820223 | 1 | 61.6135084 | 1 | 1 |
| chr1:213861854 | 1 | 56.5789474 | 1 | 1 |
| chr1:213861907 | 1 | 78.3648498 | 1 | 1 |
| chr1:213910457 | 1 | 81.1011905 | 1 | 1 |
| chr1:225962005 | 1 | 63.6029412 | 1 | 1 |
| chr1:230322908 | 1 | 77.4725275 | 1 | 1 |
| chr1:240992226 | 1 | 68.7782805 | 1 | 1 |
| chr1:241128149 | 1 | 85.3571429 | 1 | 1 |
| chr1:243916475 | 1 | 70.5032302 | 1 | 1 |
| chr10:99431109 | 1 | 2.48144712 | 1 | 1 |
| chr10:132518774 | 1 | 88.0952381 | 1 | 1 |
| chr11:366066 | 1 | 17.6022176 | 1 | 1 |
| chr11:438565 | 1 | 25.9972955 | 1 | 1 |
| chr11:438607 | 1 | 62.5 | 1 | 1 |
| chr11:726201 | 1 | 27.5641026 | 1 | 1 |
| chr11:848890 | 1 | 83.7662338 | 1 | 1 |
| chr11:848923 | 1 | 63.3333333 | 1 | 1 |
| chr11:848927 | 1 | 26.7676768 | 1 | 1 |
| chr11:848933 | 1 | 38.8888889 | 1 | 1 |
| chr11:849115 | 1 | 86.1111111 | 1 | 1 |
| chr11:849137 | 1 | 23.3766234 | 1 | 1 |
| chr11:849141 | 1 | 56.547619 | 1 | 1 |
| chr11:849142 | 1 | 86.1111111 | 1 | 1 |
| chr11:849148 | 1 | 25.8333333 | 1 | 1 |
| chr11:849149 | 1 | 78.968254 | 1 | 1 |
| chr11:17734970 | 1 | 3.57142857 | 1 | 1 |
| chr11:44566012 | 1 | 40.3743316 | 1 | 1 |
| chr11:45922046 | 1 | 63.25 | 1 | 1 |
| chr11:45922074 | 1 | 45.4285714 | 1 | 1 |
| chr11:45922076 | 1 | 42.3469388 | 1 | 1 |
| chr11:45922090 | 1 | 43.8571429 | 1 | 1 |
| chr11:45922100 | 1 | 40.9937888 | 1 | 1 |
| chr11:45922275 | 1 | 14.0873016 | 1 | 1 |
| chr11:45922276 | 1 | 18.0725524 | 1 | 1 |
| chr11:45923059 | 1 | 11.25 | 1 | 1 |
| chr11:45923077 | 1 | 11.25 | 1 | 1 |
| chr11:46381467 | 1 | 11.25 | 1 | 1 |
| chr11:64299743 | 1 | 62.3931624 | 1 | 1 |
| chr11:64299761 | 1 | 70.0854701 | 1 | 1 |
| chr11:64300844 | 1 | 55 | 1 | 1 |
| chr11:64300851 | 1 | 21.6666667 | 1 | 1 |
| chr11:64300858 | 1 | 50 | 1 | 1 |
| chr11:64300884 | 1 | 86.6666667 | 1 | 1 |
| chr11:64490631 | 1 | 88.8888889 | 1 | 1 |
| chr11:64490632 | 1 | 92.1052632 | 1 | 1 |
| chr11:64712787 | 1 | 87.8205128 | 1 | 1 |
| chr11:65355635 | 1 | 60.625 | 1 | 1 |
| chr11:65355646 | 1 | 58.3333333 | 1 | 1 |
| chr11:65355670 | 1 | 84.375 | 1 | 1 |
| chr11:65551424 | 1 | 66.7582418 | 1 | 1 |
| chr11:65551425 | 1 | 85.2941176 | 1 | 1 |
| chr11:67023317 | 1 | 5.36585366 | 1 | 1 |
| chr11:67464932 | 1 | 79.1666667 | 1 | 1 |
| chr11:67464964 | 1 | 77.0833333 | 1 | 1 |
| chr11:73264689 | 1 | 91.1654135 | 1 | 1 |
| chr11:75240135 | 1 | 50.66313 | 1 | 1 |
| chr11:85811258 | 1 | 12.8787879 | 1 | 1 |
| chr11:89593934 | 1 | 46.4285714 | 1 | 1 |
| chr11:92709117 | 1 | 35.8585859 | 1 | 1 |
| chr11:93542959 | 1 | 12.6984127 | 1 | 1 |
| chr11:114059499 | 1 | 7.17703349 | 1 | 1 |
| chr11:114059530 | 1 | 7.04545455 | 1 | 1 |
| chr11:114059757 | 1 | 13.0252101 | 1 | 1 |
| chr11:114061199 | 1 | 11.6666667 | 1 | 1 |
| chr11:114061210 | 1 | 22.5490196 | 1 | 1 |
| chr11:114061212 | 1 | 22.9166667 | 1 | 1 |
| chr11:114061215 | 1 | 22.9166667 | 1 | 1 |
| chr11:114061239 | 1 | 21.5909091 | 1 | 1 |
| chr11:114631735 | 1 | 4.16666667 | 1 | 1 |
| chr11:115504986 | 1 | 29.0625 | 1 | 1 |
| chr11:115504997 | 1 | 29.5454545 | 1 | 1 |
| chr11:115505001 | 1 | 27.173913 | 1 | 1 |
| chr11:117432347 | 1 | 88.1944444 | 1 | 1 |
| chr11:117795858 | 1 | 86.7521368 | 1 | 1 |
| chr11:118956922 | 1 | 45 | 1 | 1 |
| chr11:119182275 | 1 | 97.826087 | 1 | 1 |
| chr11:119206066 | 1 | 8.01282051 | 1 | 1 |
| chr11:119729116 | 1 | 7.5 | 1 | 1 |
| chr11:120284546 | 1 | 96.4285714 | 1 | 1 |
| chr11:124746873 | 1 | 88.4615385 | 1 | 1 |
| chr11:124746909 | 1 | 93.7728938 | 1 | 1 |
| chr11:124746913 | 1 | 89.9267399 | 1 | 1 |
| chr11:124863373 | 1 | 81.9852941 | 1 | 1 |
| chr11:124863394 | 1 | 97.0588235 | 1 | 1 |
| chr11:125263544 | 1 | 64.6153846 | 1 | 1 |
| chr11:129316134 | 1 | 96.875 | 1 | 1 |
| chr11:130070945 | 1 | 5.69727891 | 1 | 1 |
| chr11:130159700 | 1 | 93.7254902 | 1 | 1 |
| chr11:130159829 | 1 | 96.4285714 | 1 | 1 |
| chr11:130159851 | 1 | 96.4285714 | 1 | 1 |
| chr11:130159854 | 1 | 96.4285714 | 1 | 1 |
| chr11:133064200 | 1 | 41.9642857 | 1 | 1 |
| chr11:133064212 | 1 | 52.3809524 | 1 | 1 |
| chr11:133064221 | 1 | 47.8021978 | 1 | 1 |
| chr11:133064231 | 1 | 54.9450549 | 1 | 1 |
| chr11:133064233 | 1 | 55.4945055 | 1 | 1 |
| chr11:134311382 | 1 | 94.375 | 1 | 1 |
| chr12:6961918 | 1 | 41.6666667 | 1 | 1 |
| chr12:6962633 | 1 | 79.9107143 | 1 | 1 |
| chr12:6962646 | 1 | 85.1648352 | 1 | 1 |
| chr12:6962648 | 1 | 89.1826923 | 1 | 1 |
| chr12:6964182 | 1 | 69.4805195 | 1 | 1 |
| chr12:16504002 | 1 | 72.3809524 | 1 | 1 |
| chr12:16714352 | 1 | 36.6666667 | 1 | 1 |
| chr12:16714353 | 1 | 26.4423077 | 1 | 1 |
| chr12:25939377 | 1 | 99.382716 | 1 | 1 |
| chr12:32470696 | 1 | 78.4090909 | 1 | 1 |
| chr12:32646853 | 1 | 89.1812865 | 1 | 1 |
| chr12:42238116 | 1 | 6.9047619 | 1 | 1 |
| chr12:49346692 | 1 | 25.7525084 | 1 | 1 |
| chr12:51392037 | 1 | 6.9047619 | 1 | 1 |
| chr12:52903995 | 1 | 39.5454545 | 1 | 1 |
| chr12:52904021 | 1 | 43.3333333 | 1 | 1 |
| chr12:52948914 | 1 | 5.39772727 | 1 | 1 |
| chr12:96738497 | 1 | 80.1136364 | 1 | 1 |
| chr12:96738507 | 1 | 79.6969697 | 1 | 1 |
| chr12:97138576 | 1 | 23.6507937 | 1 | 1 |
| chr12:97324958 | 1 | 6.78733032 | 1 | 1 |
| chr12:97325039 | 1 | 69.0224159 | 1 | 1 |
| chr12:97607417 | 1 | 51.3636364 | 1 | 1 |
| chr12:97619683 | 1 | 20.9677419 | 1 | 1 |
| chr12:97755215 | 1 | 19.3050193 | 1 | 1 |
| chr12:97855295 | 1 | 25.7894737 | 1 | 1 |
| chr12:97924591 | 1 | 27.4884259 | 1 | 1 |
| chr12:98350733 | 1 | 69.6428571 | 1 | 1 |
| chr12:103100480 | 1 | 87.7777778 | 1 | 1 |
| chr12:103165897 | 1 | 84.8659004 | 1 | 1 |
| chr12:103165901 | 1 | 88.9520202 | 1 | 1 |
| chr12:103165909 | 1 | 89.4444444 | 1 | 1 |
| chr12:103165933 | 1 | 73.3333333 | 1 | 1 |
| chr12:108310791 | 1 | 69.6969697 | 1 | 1 |
| chr12:108310792 | 1 | 64.4385027 | 1 | 1 |
| chr12:108310807 | 1 | 59.7186701 | 1 | 1 |
| chr12:109291976 | 1 | 39.3939394 | 1 | 1 |
| chr12:109292004 | 1 | 13.6363636 | 1 | 1 |
| chr12:119258242 | 1 | 67.2222222 | 1 | 1 |
| chr12:119258280 | 1 | 46.5384615 | 1 | 1 |
| chr12:132134549 | 1 | 20.1923077 | 1 | 1 |
| chr12:132134577 | 1 | 39.5833333 | 1 | 1 |
| chr12:132134595 | 1 | 43.4210526 | 1 | 1 |
| chr12:132134596 | 1 | 49.2307692 | 1 | 1 |
| chr12:132134600 | 1 | 77.7777778 | 1 | 1 |
| chr12:132134601 | 1 | 66.8859649 | 1 | 1 |
| chr12:132134630 | 1 | 60.0478469 | 1 | 1 |
| chr13:21298314 | 1 | 2.23953262 | 1 | 1 |
| chr13:34918433 | 1 | 55.7692308 | 1 | 1 |
| chr13:35920681 | 1 | 62.7877238 | 1 | 1 |
| chr13:38494047 | 1 | 24.3506494 | 1 | 1 |
| chr13:38545892 | 1 | 54.0530303 | 1 | 1 |
| chr13:39205350 | 1 | 32.798574 | 1 | 1 |
| chr13:39300854 | 1 | 86.7521368 | 1 | 1 |
| chr13:42040772 | 1 | 4.65367965 | 1 | 1 |
| chr13:54129564 | 1 | 30.7142857 | 1 | 1 |
| chr13:54844155 | 1 | 55.2941176 | 1 | 1 |
| chr13:55129970 | 1 | 62.0300752 | 1 | 1 |
| chr13:55166399 | 1 | 20.5357143 | 1 | 1 |
| chr13:59341409 | 1 | 36.7307692 | 1 | 1 |
| chr13:70033781 | 1 | 40.922619 | 1 | 1 |
| chr13:73632816 | 1 | 11.1111111 | 1 | 1 |
| chr13:73632851 | 1 | 67.8571429 | 1 | 1 |
| chr13:78840351 | 1 | 55 | 1 | 1 |
| chr13:101796812 | 1 | 43.9958592 | 1 | 1 |
| chr13:101927978 | 1 | 72.5 | 1 | 1 |
| chr13:101933364 | 1 | 61.3649343 | 1 | 1 |
| chr13:102060417 | 1 | 44.8717949 | 1 | 1 |
| chr13:102060419 | 1 | 53.2051282 | 1 | 1 |
| chr13:102060451 | 1 | 19.0202832 | 1 | 1 |
| chr13:102068532 | 1 | 73.1664471 | 1 | 1 |
| chr13:102068551 | 1 | 29.8701299 | 1 | 1 |
| chr13:102068563 | 1 | 26.8939394 | 1 | 1 |
| chr13:102068572 | 1 | 8.71212121 | 1 | 1 |
| chr13:102390033 | 1 | 40.9722222 | 1 | 1 |
| chr13:110473172 | 1 | 21.5909091 | 1 | 1 |
| chr13:110536576 | 1 | 23.8095238 | 1 | 1 |
| chr13:110825816 | 1 | 76.8888889 | 1 | 1 |
| chr13:110830973 | 1 | 6.11111111 | 1 | 1 |
| chr13:112988667 | 1 | 75.5494505 | 1 | 1 |
| chr13:113016746 | 1 | 93.0952381 | 1 | 1 |
| chr13:113433650 | 1 | 76.3305322 | 1 | 1 |
| chr14:21069139 | 1 | 63.8888889 | 1 | 1 |
| chr14:21069177 | 1 | 83.3333333 | 1 | 1 |
| chr14:21069183 | 1 | 83.3333333 | 1 | 1 |
| chr14:22883379 | 1 | 92.1212121 | 1 | 1 |
| chr14:26553939 | 1 | 79.2366947 | 1 | 1 |
| chr14:29450905 | 1 | 25 | 1 | 1 |
| chr14:29450950 | 1 | 14.2857143 | 1 | 1 |
| chr14:29523990 | 1 | 81.5322581 | 1 | 1 |
| chr14:30391455 | 1 | 87.7777778 | 1 | 1 |
| chr14:37657127 | 1 | 88.4571429 | 1 | 1 |
| chr14:37751915 | 1 | 45.5773956 | 1 | 1 |
| chr14:37751924 | 1 | 28.8697789 | 1 | 1 |
| chr14:37751973 | 1 | 74.6376812 | 1 | 1 |
| chr14:37751985 | 1 | 56.9565217 | 1 | 1 |
| chr14:37772760 | 1 | 65 | 1 | 1 |
| chr14:37772778 | 1 | 68.7271062 | 1 | 1 |
| chr14:37772795 | 1 | 79.2857143 | 1 | 1 |
| chr14:37802591 | 1 | 45.0292398 | 1 | 1 |
| chr14:38033650 | 1 | 9.375 | 1 | 1 |
| chr14:38033658 | 1 | 24.7767857 | 1 | 1 |
| chr14:38115498 | 1 | 17.5403226 | 1 | 1 |
| chr14:38115499 | 1 | 21.5811966 | 1 | 1 |
| chr14:38115532 | 1 | 7.27272727 | 1 | 1 |
| chr14:38129918 | 1 | 41.9871795 | 1 | 1 |
| chr14:39442503 | 1 | 45.9821429 | 1 | 1 |
| chr14:44156407 | 1 | 52.2727273 | 1 | 1 |
| chr14:44156411 | 1 | 61.2299465 | 1 | 1 |
| chr14:44156431 | 1 | 48.4848485 | 1 | 1 |
| chr14:44379746 | 1 | 27.0833333 | 1 | 1 |
| chr14:44379797 | 1 | 14.2857143 | 1 | 1 |
| chr14:53956630 | 1 | 81.3888889 | 1 | 1 |
| chr14:53956642 | 1 | 81.3888889 | 1 | 1 |
| chr14:53956653 | 1 | 67.8431373 | 1 | 1 |
| chr14:53956667 | 1 | 75.8333333 | 1 | 1 |
| chr14:56506990 | 1 | 60.130719 | 1 | 1 |
| chr14:56534824 | 1 | 70.1359388 | 1 | 1 |
| chr14:56832505 | 1 | 75.9090909 | 1 | 1 |
| chr14:56992482 | 1 | 79.7619048 | 1 | 1 |
| chr14:56992483 | 1 | 63.8888889 | 1 | 1 |
| chr14:61348113 | 1 | 58.9098532 | 1 | 1 |
| chr14:62736524 | 1 | 27.7777778 | 1 | 1 |
| chr14:63064707 | 1 | 34.2592593 | 1 | 1 |
| chr14:63114304 | 1 | 51.1508951 | 1 | 1 |
| chr14:63114311 | 1 | 19.8209719 | 1 | 1 |
| chr14:63114337 | 1 | 18.1704261 | 1 | 1 |
| chr14:64741044 | 1 | 89.1812865 | 1 | 1 |
| chr14:64942593 | 1 | 81.127451 | 1 | 1 |
| chr14:64942636 | 1 | 87.0098039 | 1 | 1 |
| chr14:67241487 | 1 | 3.51588911 | 1 | 1 |
| chr14:88791865 | 1 | 12.6923077 | 1 | 1 |
| chr14:88791879 | 1 | 39.7435897 | 1 | 1 |
| chr14:104827290 | 1 | 15.6126482 | 1 | 1 |
| chr14:104895812 | 1 | 93.8888889 | 1 | 1 |
| chr14:104895822 | 1 | 93.8888889 | 1 | 1 |
| chr15:22702324 | 1 | 44.047619 | 1 | 1 |
| chr15:43776557 | 1 | 55.0420168 | 1 | 1 |
| chr15:43776576 | 1 | 53.3182504 | 1 | 1 |
| chr15:53791638 | 1 | 2.27272727 | 1 | 1 |
| chr16:64387 | 1 | 75.2525253 | 1 | 1 |
| chr16:561080 | 1 | 23.6111111 | 1 | 1 |
| chr16:561087 | 1 | 39.2857143 | 1 | 1 |
| chr16:677534 | 1 | 69.4852941 | 1 | 1 |
| chr16:2148616 | 1 | 2.53036152 | 1 | 1 |
| chr16:4260566 | 1 | 69.0384615 | 1 | 1 |
| chr16:14303673 | 1 | 22.0238095 | 1 | 1 |
| chr16:14303701 | 1 | 14.2857143 | 1 | 1 |
| chr16:14303702 | 1 | 18.6813187 | 1 | 1 |
| chr16:14303720 | 1 | 14.8351648 | 1 | 1 |
| chr16:19522006 | 1 | 5.71895425 | 1 | 1 |
| chr16:52395111 | 1 | 17.0212766 | 1 | 1 |
| chr16:52395146 | 1 | 32.5 | 1 | 1 |
| chr16:52504927 | 1 | 44.1666667 | 1 | 1 |
| chr16:52504937 | 1 | 51.0416667 | 1 | 1 |
| chr16:52504992 | 1 | 78.1400966 | 1 | 1 |
| chr16:52621872 | 1 | 21.9198565 | 1 | 1 |
| chr16:56191818 | 1 | 5.50724638 | 1 | 1 |
| chr16:58500253 | 1 | 22.6190476 | 1 | 1 |
| chr16:58500281 | 1 | 37.5 | 1 | 1 |
| chr16:58500286 | 1 | 22.6190476 | 1 | 1 |
| chr16:67666791 | 1 | 6.43939394 | 1 | 1 |
| chr16:67666821 | 1 | 11.2280702 | 1 | 1 |
| chr16:67666825 | 1 | 11.2280702 | 1 | 1 |
| chr16:67666999 | 1 | 51.2987013 | 1 | 1 |
| chr16:67667017 | 1 | 83.7662338 | 1 | 1 |
| chr16:67667021 | 1 | 55.8441558 | 1 | 1 |
| chr16:71626053 | 1 | 14.9305556 | 1 | 1 |
| chr16:84312964 | 1 | 6.77777778 | 1 | 1 |
| chr16:84312972 | 1 | 11.5555556 | 1 | 1 |
| chr16:84312978 | 1 | 9.76190476 | 1 | 1 |
| chr16:84519935 | 1 | 81.6666667 | 1 | 1 |
| chr16:84659693 | 1 | 89.5238095 | 1 | 1 |
| chr16:84842521 | 1 | 96.5517241 | 1 | 1 |
| chr16:85449391 | 1 | 86.6666667 | 1 | 1 |
| chr16:85449403 | 1 | 80 | 1 | 1 |
| chr16:85449420 | 1 | 96.6666667 | 1 | 1 |
| chr16:86467633 | 1 | 45 | 1 | 1 |
| chr16:88291496 | 1 | 97.3684211 | 1 | 1 |
| chr16:88302508 | 1 | 93.9285714 | 1 | 1 |
| chr16:88737568 | 1 | 14.3589744 | 1 | 1 |
| chr17:4263552 | 1 | 3.67063492 | 1 | 1 |
| chr17:4449947 | 1 | 97.826087 | 1 | 1 |
| chr17:7044006 | 1 | 6.11888112 | 1 | 1 |
| chr17:17403801 | 1 | 79.6153846 | 1 | 1 |
| chr17:17403831 | 1 | 58.3333333 | 1 | 1 |
| chr17:19379152 | 1 | 76.1904762 | 1 | 1 |
| chr17:19379174 | 1 | 84.1666667 | 1 | 1 |
| chr17:19379191 | 1 | 85 | 1 | 1 |
| chr17:29176890 | 1 | 10.4166667 | 1 | 1 |
| chr17:29565877 | 1 | 64.2888889 | 1 | 1 |
| chr17:29565896 | 1 | 60.5756359 | 1 | 1 |
| chr17:29565920 | 1 | 27.7058824 | 1 | 1 |
| chr17:31561710 | 1 | 68.2389937 | 1 | 1 |
| chr17:41527810 | 1 | 26.0504202 | 1 | 1 |
| chr17:42951031 | 1 | 92.8205128 | 1 | 1 |
| chr17:43720600 | 1 | 93.75 | 1 | 1 |
| chr17:48892861 | 1 | 3.00679327 | 1 | 1 |
| chr17:65625809 | 1 | 58.7412587 | 1 | 1 |
| chr17:68870337 | 1 | 21.8253968 | 1 | 1 |
| chr17:74213330 | 1 | 5.71895425 | 1 | 1 |
| chr17:74961013 | 1 | 52.2222222 | 1 | 1 |
| chr17:74961036 | 1 | 78.4188034 | 1 | 1 |
| chr17:74972136 | 1 | 22.9166667 | 1 | 1 |
| chr17:79794767 | 1 | 65.8175081 | 1 | 1 |
| chr17:81397114 | 1 | 5.59685641 | 1 | 1 |
| chr17:81397126 | 1 | 6.9047619 | 1 | 1 |
| chr17:81397150 | 1 | 5.74534161 | 1 | 1 |
| chr17:81514240 | 1 | 11.6883117 | 1 | 1 |
| chr17:81902419 | 1 | 65.7142857 | 1 | 1 |
| chr17:81902429 | 1 | 61.1111111 | 1 | 1 |
| chr17:81902430 | 1 | 61.2938596 | 1 | 1 |
| chr17:81902433 | 1 | 73.8095238 | 1 | 1 |
| chr17:81902434 | 1 | 64.7660819 | 1 | 1 |
| chr17:81902440 | 1 | 66.2698413 | 1 | 1 |
| chr17:81902441 | 1 | 72.1362229 | 1 | 1 |
| chr18:6929505 | 1 | 7.94117647 | 1 | 1 |
| chr18:7327489 | 1 | 2.08333333 | 1 | 1 |
| chr18:7327495 | 1 | 2.08333333 | 1 | 1 |
| chr18:32928012 | 1 | 70.6521739 | 1 | 1 |
| chr18:32928017 | 1 | 82 | 1 | 1 |
| chr18:32928020 | 1 | 65.1895044 | 1 | 1 |
| chr18:32928024 | 1 | 83.8271605 | 1 | 1 |
| chr18:33167723 | 1 | 34.0909091 | 1 | 1 |
| chr18:33167727 | 1 | 29.5454545 | 1 | 1 |
| chr18:33225321 | 1 | 28.5714286 | 1 | 1 |
| chr18:33317997 | 1 | 54.6558704 | 1 | 1 |
| chr18:33318065 | 1 | 32.8671329 | 1 | 1 |
| chr18:33570344 | 1 | 55 | 1 | 1 |
| chr18:34609374 | 1 | 83.2167832 | 1 | 1 |
| chr18:34639003 | 1 | 70.7142857 | 1 | 1 |
| chr18:38714972 | 1 | 16.6666667 | 1 | 1 |
| chr18:38714994 | 1 | 21.6666667 | 1 | 1 |
| chr18:43102815 | 1 | 85.645933 | 1 | 1 |
| chr18:47748194 | 1 | 70.7142857 | 1 | 1 |
| chr18:59148758 | 1 | 95.4545455 | 1 | 1 |
| chr18:59148765 | 1 | 97.2222222 | 1 | 1 |
| chr18:63207225 | 1 | 60.4166667 | 1 | 1 |
| chr18:63207233 | 1 | 73.75 | 1 | 1 |
| chr18:63207260 | 1 | 41.25 | 1 | 1 |
| chr18:71409912 | 1 | 59 | 1 | 1 |
| chr18:71652314 | 1 | 38.4057971 | 1 | 1 |
| chr18:71652318 | 1 | 39.2018779 | 1 | 1 |
| chr18:72662276 | 1 | 8.68486352 | 1 | 1 |
| chr18:72677583 | 1 | 26.6590389 | 1 | 1 |
| chr18:76446564 | 1 | 42.8571429 | 1 | 1 |
| chr18:76446573 | 1 | 38.8888889 | 1 | 1 |
| chr18:76446574 | 1 | 40.8333333 | 1 | 1 |
| chr18:76446612 | 1 | 28.1818182 | 1 | 1 |
| chr18:76472706 | 1 | 86.5473236 | 1 | 1 |
| chr18:76580424 | 1 | 61.9047619 | 1 | 1 |
| chr18:76590712 | 1 | 33.5664336 | 1 | 1 |
| chr18:76590755 | 1 | 20.6552707 | 1 | 1 |
| chr18:76590771 | 1 | 50.6256517 | 1 | 1 |
| chr18:77020807 | 1 | 56.6666667 | 1 | 1 |
| chr18:77132839 | 1 | 4.81601732 | 1 | 1 |
| chr18:79334799 | 1 | 85.4166667 | 1 | 1 |
| chr19:460731 | 1 | 8.71212121 | 1 | 1 |
| chr19:460763 | 1 | 8.11688312 | 1 | 1 |
| chr19:511206 | 1 | 49.4152047 | 1 | 1 |
| chr19:511219 | 1 | 48.7616099 | 1 | 1 |
| chr19:537165 | 1 | 88.7362637 | 1 | 1 |
| chr19:1082036 | 1 | 53.4965035 | 1 | 1 |
| chr19:1082073 | 1 | 70.9486166 | 1 | 1 |
| chr19:1082079 | 1 | 50.5928854 | 1 | 1 |
| chr19:1083180 | 1 | 81.043956 | 1 | 1 |
| chr19:2624624 | 1 | 24.2857143 | 1 | 1 |
| chr19:4147445 | 1 | 20.7282913 | 1 | 1 |
| chr19:4566632 | 1 | 96.6666667 | 1 | 1 |
| chr19:4566647 | 1 | 96.875 | 1 | 1 |
| chr19:4955146 | 1 | 81.7813765 | 1 | 1 |
| chr19:6464556 | 1 | 88.8888889 | 1 | 1 |
| chr19:6551811 | 1 | 76.4285714 | 1 | 1 |
| chr19:6583884 | 1 | 60.3896104 | 1 | 1 |
| chr19:7115981 | 1 | 51.0882353 | 1 | 1 |
| chr19:7904264 | 1 | 4.85805861 | 1 | 1 |
| chr19:9953412 | 1 | 76.1904762 | 1 | 1 |
| chr19:9953424 | 1 | 83.6870027 | 1 | 1 |
| chr19:9961699 | 1 | 95.8791209 | 1 | 1 |
| chr19:9966503 | 1 | 70.3266788 | 1 | 1 |
| chr19:9966579 | 1 | 58.4586466 | 1 | 1 |
| chr19:9966586 | 1 | 62.6984127 | 1 | 1 |
| chr19:9997385 | 1 | 58.3333333 | 1 | 1 |
| chr19:10625441 | 1 | 7.60263053 | 1 | 1 |
| chr19:10625699 | 1 | 12.9554656 | 1 | 1 |
| chr19:11418683 | 1 | 13.3928571 | 1 | 1 |
| chr19:13099153 | 1 | 85.6037152 | 1 | 1 |
| chr19:13248768 | 1 | 83.974359 | 1 | 1 |
| chr19:14433259 | 1 | 5.25641026 | 1 | 1 |
| chr19:15195814 | 1 | 6.69642857 | 1 | 1 |
| chr19:15195823 | 1 | 10.5555556 | 1 | 1 |
| chr19:15195830 | 1 | 10.1010101 | 1 | 1 |
| chr19:15195831 | 1 | 30.5194805 | 1 | 1 |
| chr19:15195838 | 1 | 10.7142857 | 1 | 1 |
| chr19:15195844 | 1 | 6.9047619 | 1 | 1 |
| chr19:15195847 | 1 | 12.6984127 | 1 | 1 |
| chr19:15195851 | 1 | 21.0653753 | 1 | 1 |
| chr19:17041847 | 1 | 51.6666667 | 1 | 1 |
| chr19:17251851 | 1 | 91.875 | 1 | 1 |
| chr19:28293220 | 1 | 65.625 | 1 | 1 |
| chr19:29892627 | 1 | 95.6481481 | 1 | 1 |
| chr19:33260980 | 1 | 91.6666667 | 1 | 1 |
| chr19:33393584 | 1 | 88.7362637 | 1 | 1 |
| chr19:36008846 | 1 | 53.2388664 | 1 | 1 |
| chr19:36009562 | 1 | 4.94623656 | 1 | 1 |
| chr19:42242259 | 1 | 4.20634921 | 1 | 1 |
| chr19:43757320 | 1 | 30 | 1 | 1 |
| chr19:44796859 | 1 | 36.8421053 | 1 | 1 |
| chr19:45444316 | 1 | 12.6984127 | 1 | 1 |
| chr19:45598430 | 1 | 65.1515152 | 1 | 1 |
| chr19:46016118 | 1 | 34.375 | 1 | 1 |
| chr19:46016119 | 1 | 58.1196581 | 1 | 1 |
| chr19:46016123 | 1 | 34.375 | 1 | 1 |
| chr19:46016124 | 1 | 63.6752137 | 1 | 1 |
| chr19:46016125 | 1 | 26.0416667 | 1 | 1 |
| chr19:46016126 | 1 | 32.0512821 | 1 | 1 |
| chr19:46016130 | 1 | 14.5833333 | 1 | 1 |
| chr19:46016136 | 1 | 21.875 | 1 | 1 |
| chr19:46016138 | 1 | 18.75 | 1 | 1 |
| chr19:46016139 | 1 | 30.952381 | 1 | 1 |
| chr19:46016142 | 1 | 41.4529915 | 1 | 1 |
| chr19:46016144 | 1 | 38.5416667 | 1 | 1 |
| chr19:46016145 | 1 | 62.7090301 | 1 | 1 |
| chr19:46016146 | 1 | 26.6666667 | 1 | 1 |
| chr19:46016147 | 1 | 37.6068376 | 1 | 1 |
| chr19:46016148 | 1 | 30.2083333 | 1 | 1 |
| chr19:46016149 | 1 | 28.2051282 | 1 | 1 |
| chr19:46016465 | 1 | 9.82142857 | 1 | 1 |
| chr19:48752569 | 1 | 12.5 | 1 | 1 |
| chr19:48752624 | 1 | 8.98334794 | 1 | 1 |
| chr19:48752637 | 1 | 9.44170772 | 1 | 1 |
| chr19:48753115 | 1 | 7.39348371 | 1 | 1 |
| chr19:48753117 | 1 | 11.1428571 | 1 | 1 |
| chr19:48753159 | 1 | 15.7509158 | 1 | 1 |
| chr19:48753363 | 1 | 78.604119 | 1 | 1 |
| chr19:48753390 | 1 | 47.2222222 | 1 | 1 |
| chr19:50417522 | 1 | 36.6071429 | 1 | 1 |
| chr19:50418925 | 1 | 76.8115942 | 1 | 1 |
| chr19:51030848 | 1 | 2.38095238 | 1 | 1 |
| chr19:51077450 | 1 | 11.8055556 | 1 | 1 |
| chr19:52028350 | 1 | 4.71491228 | 1 | 1 |
| chr19:53991315 | 1 | 23.0113636 | 1 | 1 |
| chr19:55374611 | 1 | 58.5227273 | 1 | 1 |
| chr19:55554857 | 1 | 68.627451 | 1 | 1 |
| chr2:3303753 | 1 | 6.97115385 | 1 | 1 |
| chr2:4107720 | 1 | 10.1010101 | 1 | 1 |
| chr2:4798818 | 1 | 43.75 | 1 | 1 |
| chr2:6987052 | 1 | 89.2045455 | 1 | 1 |
| chr2:7032109 | 1 | 80.952381 | 1 | 1 |
| chr2:8457740 | 1 | 97.6190476 | 1 | 1 |
| chr2:14507507 | 1 | 31.884058 | 1 | 1 |
| chr2:25190664 | 1 | 53.90625 | 1 | 1 |
| chr2:47369968 | 1 | 20.4273504 | 1 | 1 |

Sample Classification

In order to obtain the highest number of classified samples for the analysis of differential methylation, the results from the RNAseq and DNA methylation analysis were combined with a hierarchical approach in which RNAseq-based classification was used as principal classifier. For samples in which RNAseq-based classification was equivocal or in which RNAseq was not performed the SCLC subtype based on the DNA methylation-based predictor was used to gather a classification for the majority of samples.

Data Analysis

All analysis was performed in R v4.1.1. Binning of the genome was performed based on the BSgenome.Hsapiens.NCBI.GRCh38 database using a tile width of 100.000 bp cutting the last tile of each chromosome. DNA methylation across each tile was averaged excluding missing data. To analyse the genome-wide methylation per subtype, the mean methylation per tile per sample was further averaged per subtype. The rolling average of 500 bins (=50 Mbp) was calculated using the ‘rollmean’ function in the R zoo package.

In order to annotate the methylation sites to regions in the genome associated with genes, the annotatr package was used. The following regions were annotated based on the GRCh38 genome: “hg38_genes_promoters”, “hg38_genes_exons”, “hg38_genes_introns”, “hg38_genes_1to5kb”, “hg38_genes_5UTRs”, “hg38_genes_intergenic”, “hg38_genes_3UTRs”, “hg38_genes_firstexons”, “hg38_genes_intronexonboundaries”, “hg38_genes_exonintronboundaries”.

Association of DNA methylation sites or regions was performed using pROC. Cut-offs were calculated using Youden's J and sensitivity and specificity has been calculated based on the pre-calculated cut-off. For the calculations of differences, unless otherwise highlighted. Wilcoxon test was used with FDR correction for multiple testing using rstatix.

Figures were created using ggplot2 or ComplexHeatmap and Venn Diagrams with ggVennDiagram.

Xenograft Samples

Patient-derived xenograft models were prepared as published previously. Briefly, patients with a confirmed diagnosis of SCLC consented to the LAB10-0442 protocol at the MD Anderson Cancer Center. Circulating tumor cells (CTCs) were isolated using the RosetteSep CTC enrichment cocktail and isolated CTCs were mixed 1:1 with Matrigel and injected subcutaneously into the flank of NOD.Cg-Prkdcscid Il2rgtm1Wj1/SzJ mice.

Cell Line Samples

Cell lines were cultivated as published previously. Contamination with mycoplasma was tested regularly.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The following references, and those cited elsewhere herein, to the extent that they provide exemplary procedural or other details supplementary to those set forth herein, are specifically incorporated herein by reference.
1. Byers, L. A. & Rudin, C. M. Small cell lung cancer: where do we go from here? Cancer 121, 664-672 (2015).
2. Horn. L., et al. First-Line Atezolizumab plus Chemotherapy in Extensive-Stage Small-Cell Lung Cancer. N Engl J Med 379, 2220-2229 (2018).
3. Paz-Ares, L., et al. Durvalumab plus platinum-etoposide versus platinum-etoposide in first-line treatment of extensive-stage small-cell lung cancer (CASPIAN): a randomised, controlled, open-label, phase 3 trial. Lancet 394, 1929-1939 (2019).
4. Uprety, D., Remon, J. & Adjei, A. A. All That Glitters Is Not Gold: The Story of Rovalpituzumab Tesirine in SCLC. J Thorac Oncol 16, 1429-1433 (2021).
5. Byers, L. A., Chiappori, A. & Smit, M.-A. D. Phase 1 study of AMG 119, a chimeric antigen receptor (CAR) T cell therapy targeting DLL3, in patients with relapsed/refractory small cell lung cancer (SCLC). J. Clin. Oncol. 37, 1-TPS8576 (2019).
6. Hipp, S., et al. A Bispecific DLL3/CD3 IgG-Like T-Cell Engaging Antibody Induces Antitumor Responses in Small Cell Lung Cancer. Clin Cancer Res 26, 5258-5268 (2020).
7. Pietanza, M. C., et al. Randomized, Double-Blind, Phase II Study of Temozolomide in Combination With Either Veliparib or Placebo in Patients With Relapsed-Sensitive or Refractory Small-Cell Lung Cancer. J Clin Oncol 36, 2386-2394 (2018).
8. Howlader, N., et al. The Effect of Advances in Lung-Cancer Treatment on Population Mortality. N Engl J Med 383, 640-649 (2020).
9. Rudin, C. M., et al. Molecular subtypes of small cell lung cancer: a synthesis of human and mouse model data. Nat Rev Cancer 19, 289-297 (2019).
10. Baine, M. K., et al. SCLC Subtypes Defined by ASCL1. NEUROD1, POU2F3, and YAP1: A Comprehensive Immunohistochemical and Histopathologic Characterization. J Thorac Oncol 15, 1823-1835 (2020).
11. Gay, C. M., et al. Patterns of transcription factor programs and immune pathway activation define four major subtypes of SCLC with distinct therapeutic vulnerabilities. Cancer Cell 39, 346-360 e347 (2021).
12. Schwendenwein, A., et al. Molecular profiles of small cell lung cancer subtypes: therapeutic implications. Mol Ther Oncolytics 20, 470-483 (2021).
13. National Comprehensive Cancer Network. Small Cell Lung Cancer (2022). Vol. 2021 (2021).
14. Kalari, S., Jung, M., Kernstine, K. H., Takahashi, T. & Pfeifer, G. P. The DNA methylation landscape of small cell lung cancer suggests a differentiation defect of neuroendocrine cells. Oncogene 32, 3559-3568 (2013).
15. Krushkal, J., et al. Epigenome-wide DNA methylation analysis of small cell lung cancer cell lines suggests potential chemotherapy targets. Clin Epigenetics 12, 93 (2020).
16. Zhang, Y., et al. Pan-cancer circulating tumor DNA detection in over 10,000 Chinese patients. Nat Commun 12, 11 (2021).
17. Gaga, M., et al. Validation of Lung EpiCheck, a novel methylation-based blood assay, for the detection of lung cancer in European and Chinese high-risk individuals. Eur Respir J 57(2021).
18. George, J., et al. Comprehensive genomic profiles of small cell lung cancer. Nature 524, 47-53 (2015).
19. Tlemsani, C., et al. SCLC-CellMiner: A Resource for Small Cell Lung Cancer Cell Line Genomics and Pharmacology Based on Genomic Signatures. Cell Rep 33, 108296 (2020).
20. Iorio, F., et al. A Landscape of Pharmacogenomic Interactions in Cancer. Cell 166, 740-754 (2016).
21. Van Paemel, R., et al. Genome-wide study of the effect of blood collection tubes on the cell-free DNA methylome. Epigenetics 16, 797-807 (2021).
22. Tang, M., et al. The histologic phenotype of lung cancers is associated with transcriptomic features rather than genomic characteristics. Nat Commun 12, 7081 (2021).
23. Sato, Y., et al. Integrated Immunohistochemical Study on Small-Cell Carcinoma of the Lung Focusing on Transcription and Co-Transcription Factors. Diagnostics (Basel) 10(2020).
24. Stewart, C. A., et al. Single-cell analyses reveal increased intratumoral heterogeneity after the onset of therapy resistance in small-cell lung cancer. Nat Cancer 1, 423-436 (2020).
25. Poirier, J. T., et al. DNA methylation in small cell lung cancer defines distinct disease subtypes and correlates with high expression of EZH2. Oncogene 34, 5869-5878 (2015).
26. Lin. S. H., et al. Genes suppressed by DNA methylation in non-small cell lung cancer reveal the epigenetics of epithelial-mesenchymal transition. BMC Genomics 15, 1079 (2014).
27. Wang, Z., et al. Complex impact of DNA methylation on transcriptional dysregulation across 22 human cancer types. Nucleic Acids Res 48, 2287-2302 (2020).
28. Liu, M. C., et al. Sensitive and specific multi-cancer detection and localization using methylation signatures in cell-free DNA. Ann Oncol 31, 745-759 (2020).
29. Kilgour, E., Rothwell, D. G., Brady, G. & Dive, C. Liquid Biopsy-Based Biomarkers of Treatment Response and Resistance. Cancer Cell 37, 485-495 (2020).

What is claimed:

1. A method of treating a subject for small cell lung cancer (SCLC), the method comprising administering a BCL2 inhibitor or a DLL3-targeted therapy to a subject having tumor DNA with differential methylation at one or more methylation sites selected from the methyation sites of Table 2, Table 7, Table 15, Table 20, and Table 27 compared to a reference or control sample.

2. The method of claim 1, wherein the one or more methylation sites are 2 or more methylation sites selected from the methyation sites of Table 2, Table 7, Table 15, Table 20, and Table 27.

3. The method of claim 1, wherein the one or more methylation sites are 3 or more methylation sites selected from the methyation sites of Table 2, Table 7, Table 15, Table 20, and Table 27.

4. The method of claim 1, wherein the one or more methylation sites are 4 or more methylation sites selected from the methyation sites of Table 2, Table 7, Table 15, Table 20, and Table 27.

5. The method of claim 1, wherein the one or more methylation sites are 5 or more methylation sites selected from the methyation sites of Table 2, Table 7, Table 15, Table 20, and Table 27.

6. The method of claim 1, wherein the one or more methylation sites are 10 or more methylation sites selected from the methyation sites of Table 2, Table 7, Table 15, Table 20, and Table 27.

7. The method of claim 1, wherein the one or more methylation sites are twenty or more methylation sites selected from the methyation sites of Table 2, Table 7, Table 15, Table 20, and Table 27.

8. The method of claim 1, wherein the one or more methylation sites are fifty or more methylation sites selected from the methyation sites of Table 2, Table 7, Table 15, Table 20, and Table 27.

9. The method of claim 1, wherein the one or more methylation sites are one or more methylation sites of Table 2.

10. The method of claim 1, wherein the one or more methylation sites are 2 or more methylation sites of Table 2.

11. The method of claim 1, wherein the one or more methylation sites are 3 or more methylation sites of Table 2.

12. The method of claim 1, wherein the one or more methylation sites are 4 or more methylation sites of Table 2.

13. The method of claim 1, wherein the one or more methylation sites are 5 or more methylation sites of Table 2.

14. The method of claim 1, wherein the one or more methylation sites are 10 or more methylation sites of Table 2.

15. The method of claim 1, wherein the one or more methylation sites are all of the methylation sites of Table 2.

16. The method of claim 1, wherein the one or more methylation sites are one or more methylation sites of Table 7.

17. The method of claim 1, wherein the one or more methylation sites are 2 or more methylation sites of Table 7.

18. The method of claim 1, wherein the one or more methylation sites are 3 or more methylation sites of Table 7.

19. The method of claim 1, wherein the one or more methylation sites are 4 or more methylation sites of Table 7.

20. The method of claim 1, wherein the one or more methylation sites are one or more methylation sites of Table 7.

21. The method of claim 1, wherein the one or more methylation sites are 10 or more methylation sites of Table 7.

22. The method of claim 1, wherein the one or more methylation sites are all of the methylation sites of Table 7.

23. The method of claim 1, wherein the one or more methylation sites are one or more methylation sites of Table 15.

24. The method of claim 1, wherein the one or more methylation sites are 2 or more methylation sites of Table 15.

25. The method of claim 1, wherein the one or more methylation sites are 3 or more methylation sites of Table 15.

26. The method of claim 1, wherein the one or more methylation sites are 4 or more methylation sites of Table 15.

27. The method of claim 1, wherein the one or more methylation sites are 5 or more methylation sites of Table 15.

28. The method of claim 1, wherein the one or more methylation sites are 10 or more methylation sites of Table 15.

29. The method of claim 1, wherein the one or more methylation sites are all of the methylation sites of Table 15.

30. The method of claim 1, wherein the one or more methylation sites are one or more methylation sites of Table 20.

31. The method of claim 1, wherein the one or more methylation sites are 2 or more methylation sites of Table 20.

32. The method of claim 1, wherein the one or more methylation sites are 3 or more methylation sites of Table 20.

33. The method of claim 1, wherein the one or more methylation sites are 4 or more methylation sites of Table 20.

34. The method of claim 1, wherein the one or more methylation sites are 5 or more methylation sites of Table 20.

35. The method of claim 1, wherein the one or more methylation sites are 10 or more methylation sites of Table 20.

36. The method of claim 1, wherein the one or more methylation sites are all of the methylation sites of Table 20.

37. The method of claim 1, wherein the one or more methylation sites are one or more methylation sites of Table 27.

38. The method of claim 1, wherein the one or more methylation sites are 2 or more methylation sites of Table 27.

39. The method of claim 1, wherein the one or more methylation sites are 3 or more methylation sites of Table 27.

40. The method of claim 1, wherein the one or more methylation sites are 4 or more methylation sites of Table 27.

41. The method of claim 1, wherein the one or more methylation sites are 5 or more methylation sites of Table 27.

42. The method of claim 1, wherein the one or more methylation sites are 10 or more methylation sites of Table 27.

43. The method of claim 1, wherein the one or more methylation sites are all of the methylation sites of Table 27.

44. The method of any of claims 1-43, wherein the tumor DNA is ctDNA.

45. The method of any of claims 1-22, wherein the method comprises administering to the subject the BCL2 inhibitor.

46. The method of claim 45, wherein the BCL2 inhibitor is ABT-737 or navitoclax.

47. The method of any of claims 1-45, wherein the method comprises administering to the subject the DLL3-targeted therapeutic.

48. The method of claim 47, wherein the DLL3-targeted therapeutic comprises an anti-DLL3 antibody or fragment thereof.

49. The method of claim 48, wherein the anti-DLL3 antibody or fragment thereof is rovalpituzumab.

50. The method of claim 47, wherein the DLL3-targeted therapeutic is an antibody-drug conjugate.

51. The method of claim 50, wherein the antibody-drug conjugate is rovalpituzumab tesirine.

52. The method of claim 47, wherein the DLL3-targeted therapeutic is a DLL3-targeted cellular therapy.

53. The method of claim 52, wherein the DLL3-targeted cellular therapy is a DLL3-targeted chimeric antigen receptor T-cell.

54. The method of any of claims 1-52, further comprising administering to the subject an additional cancer therapy.

55. The method of claim 54, wherein the additional cancer therapy comprises chemotherapy, radiotherapy, immunotherapy, or a combination thereof.

56. The method of any of claims 1-55, wherein the subject was previously treated for SCLC.

57. The method of claim 1-56, wherein the subject was resistant to the previous treatment.

58. The method of any of claims 1-57, wherein the subject was determined to have SCLC-A based on the analysis of the tumor DNA.

59. The method of any of claims 1-58, wherein the subject was further determined, from the analysis of the tumor DNA from the subject, to have differential methylation of one or more methylation sites of Table 13 compared to the reference or control sample.

60. The method of any of claims 1-59, wherein the reference or control sample is a DNA sample obtained from healthy cells from the subject.

61. The method of any of claims 1-59, wherein the reference or control sample is a DNA sample obtained from healthy cells from a reference subject.

62. A method of treating a subject for SCLC, the method comprising administering an AURK inhibitor to a subject having tumor DNA with differential methylation at one or more methylation sites selected from the methylation sites of Table 3, Table 8, Table 16, Table 21, and Table 28 compared to a reference or control sample.

63. The method of claim 62, wherein the one or more methylation sites are 2 or more methylation sites selected from the methyation sites of Table 3, Table 8, Table 16, Table 21, and Table 28.

64. The method of claim 62, wherein the one or more methylation sites are 3 or more methylation sites selected from the methyation sites of Table 3, Table 8, Table 16, Table 21, and Table 28.

65. The method of claim 62, wherein the one or more methylation sites are 4 or more methylation sites selected from the methyation sites of Table 3, Table 8, Table 16, Table 21, and Table 28.

66. The method of claim 62, wherein the one or more methylation sites are 5 or more methylation sites selected from the methyation sites of Table 3, Table 8, Table 16, Table 21, and Table 28.

67. The method of claim 62, wherein the one or more methylation sites are 10 or more methylation sites selected from the methyation sites of Table 3, Table 8, Table 16, Table 21, and Table 28.

68. The method of claim 62, wherein the one or more methylation sites are twenty or more methylation sites selected from the methyation sites of Table 3, Table 8, Table 16, Table 21, and Table 28.

69. The method of claim 62, wherein the one or more methylation sites are fifty or more methylation sites selected from the methyation sites of Table 3, Table 8, Table 16, Table 21, and Table 28.

70. The method of claim 62, wherein the one or more methylation sites are one or more methylation sites of Table 3.

71. The method of claim 62, wherein the one or more methylation sites are 2 or more methylation sites of Table 3.

72. The method of claim 62, wherein the one or more methylation sites are 3 or more methylation sites of Table 3.

73. The method of claim 62, wherein the one or more methylation sites are 4 or more methylation sites of Table 3.

74. The method of claim 62, wherein the one or more methylation sites are 5 or more methylation sites of Table 3.

75. The method of claim 62, wherein the one or more methylation sites are 10 or more methylation sites of Table 3.

76. The method of claim 62, wherein the one or more methylation sites are all of the methylation sites of Table 3.

77. The method of claim 62, wherein the one or more methylation sites are one or more methylation sites of Table 8.

78. The method of claim 62, wherein the one or more methylation sites are 2 or more methylation sites of Table 8.

79. The method of claim 62, wherein the one or more methylation sites are 3 or more methylation sites of Table 8.

80. The method of claim 62, wherein the one or more methylation sites are 4 or more methylation sites of Table 8.

81. The method of claim 62, wherein the one or more methylation sites are 5 or more methylation sites of Table 8.

82. The method of claim 62, wherein the one or more methylation sites are 10 or more methylation sites of Table 8.

83. The method of claim 62, wherein the one or more methylation sites are all of the methylation sites of Table 8.

84. The method of claim 62, wherein the one or more methylation sites are one or more methylation sites of Table 16.

85. The method of claim 62, wherein the one or more methylation sites are 2 or more methylation sites of Table 16.

86. The method of claim 62, wherein the one or more methylation sites are 3 or more methylation sites of Table 16.

87. The method of claim 62, wherein the one or more methylation sites are 4 or more methylation sites of Table 16.

88. The method of claim 62, wherein the one or more methylation sites are 5 or more methylation sites of Table 16.

89. The method of claim 62, wherein the one or more methylation sites are 10 or more methylation sites of Table 16.

90. The method of claim 62, wherein the one or more methylation sites are all of the methylation sites of Table 16.

91. The method of claim 62, wherein the one or more methylation sites are one or more methylation sites of Table 21.

92. The method of claim 62, wherein the one or more methylation sites are 2 or more methylation sites of Table 21.

93. The method of claim 62, wherein the one or more methylation sites are 3 or more methylation sites of Table 21.

94. The method of claim 62, wherein the one or more methylation sites are 4 or more methylation sites of Table 21.

95. The method of claim 62, wherein the one or more methylation sites are 5 or more methylation sites of Table 21.

96. The method of claim 62, wherein the one or more methylation sites are 10 or more methylation sites of Table 21.

97. The method of claim 62, wherein the one or more methylation sites are all of the methylation sites of Table 21.

98. The method of claim 62, wherein the one or more methylation sites are one or more methylation sites of Table 28.

99. The method of claim 62, wherein the one or more methylation sites are 2 or more methylation sites of Table 28.

100. The method of claim 62, wherein the one or more methylation sites are 3 or more methylation sites of Table 28.

101. The method of claim 62, wherein the one or more methylation sites are 4 or more methylation sites of Table 28.

102. The method of claim 62, wherein the one or more methylation sites are 5 or more methylation sites of Table 28.

103. The method of claim 62, wherein the one or more methylation sites are 10 or more methylation sites of Table 28.

104. The method of claim 62, wherein the one or more methylation sites are all of the methylation sites of Table 28.

105. The method of any of claims 62-104, wherein the tumor DNA is ctDNA.

106. The method of any of claims 62-105, wherein the AURK inhibitor is CYC-116, alisertib, or AS-703569.

107. The method of any of claims 62-106, further comprising administering to the subject an additional cancer therapy.

108. The method of claim 107, wherein the additional cancer therapy comprises chemotherapy, radiotherapy, immunotherapy, or a combination thereof.

109. The method of any of claims 62-108, wherein the subject was previously treated for SCLC.

110. The method of claim 109, wherein the subject was resistant to the previous treatment.

111. The method of any of claims 62-110, wherein the subject was determined to have SCLC-N based on the analysis of the tumor DNA.

112. The method of any of claims 62-111, wherein the subject was further determined, from the analysis of the tumor DNA from the subject, to have differential methylation of one or more methylation sites of Table 13 compared to the reference or control sample.

113. The method of any of claims 62-112, wherein the reference or control sample is a DNA sample obtained from healthy cells from the subject.

114. The method of any of claims 62-112, wherein the reference or control sample is a DNA sample obtained from healthy cells from a reference subject.

115. A method of treating a subject for SCLC, the method comprising administering a platinum-based chemotherapeutic agent, a PARP inhibitor, an anti-metabolite, or a nucleoside analog to a subject having tumor DNA with differential methylation at one or more methylation sites selected from the methylation sites of Table 4, Table 9, Table 17, Table 22, and Table 29 compared to a reference or control sample.

116. The method of claim 115, wherein the one or more methylation sites are 2 or more methylation sites selected from the methyation sites of Table 4, Table 9, Table 17, Table 22, and Table 29.

117. The method of claim 115, wherein the one or more methylation sites are 3 or more methylation sites selected from the methyation sites of Table 4, Table 9, Table 17, Table 22, and Table 29.

118. The method of claim 115, wherein the one or more methylation sites are 4 or more methylation sites selected from the methyation sites of Table 4, Table 9, Table 17, Table 22, and Table 29.

119. The method of claim 115, wherein the one or more methylation sites are 5 or more methylation sites selected from the methyation sites of Table 4, Table 9, Table 17, Table 22, and Table 29.

120. The method of claim 115, wherein the one or more methylation sites are 10 or more methylation sites selected from the methyation sites of Table 4, Table 9, Table 17, Table 22, and Table 29.

121. The method of claim 115, wherein the one or more methylation sites are twenty or more methylation sites selected from the methyation sites of Table 4, Table 9, Table 17, Table 22, and Table 29.

122. The method of claim 115, wherein the one or more methylation sites are fifty or more methylation sites selected from the methyation sites of Table 4, Table 9, Table 17, Table 22, and Table 29.

123. The method of claim 115, wherein the one or more methylation sites are one or more methylation sites of Table 4.

124. The method of claim 115, wherein the one or more methylation sites are 2 or more methylation sites of Table 4.

125. The method of claim 115, wherein the one or more methylation sites are 3 or more methylation sites of Table 4.

126. The method of claim 115, wherein the one or more methylation sites are 4 or more methylation sites of Table 4.

127. The method of claim 115, wherein the one or more methylation sites are 5 or more methylation sites of Table 4.

128. The method of claim 115, wherein the one or more methylation sites are 10 or more methylation sites of Table 4.

129. The method of claim 115, wherein the one or more methylation sites are all of the methylation sites of Table 4.

130. The method of claim 115, wherein the one or more methylation sites are one or more methylation sites of Table 9.

131. The method of claim 115, wherein the one or more methylation sites are 2 or more methylation sites of Table 9.

132. The method of claim 115, wherein the one or more methylation sites are 3 or more methylation sites of Table 9.

133. The method of claim 115, wherein the one or more methylation sites are 4 or more methylation sites of Table 9.

134. The method of claim 115, wherein the one or more methylation sites are 5 or more methylation sites of Table 9.

135. The method of claim 115, wherein the one or more methylation sites are 10 or more methylation sites of Table 9.

136. The method of claim 115, wherein the one or more methylation sites are all of the methylation sites of Table 9.

137. The method of claim 115, wherein the one or more methylation sites are one or more methylation sites of Table 17.

138. The method of claim 115, wherein the one or more methylation sites are 2 or more methylation sites of Table 17.

139. The method of claim 115, wherein the one or more methylation sites are 3 or more methylation sites of Table 17.

140. The method of claim 115, wherein the one or more methylation sites are 4 or more methylation sites of Table 17.

141. The method of claim 115, wherein the one or more methylation sites are 5 or more methylation sites of Table 17.

142. The method of claim 115, wherein the one or more methylation sites are 10 or more methylation sites of Table 17.

143. The method of claim 115, wherein the one or more methylation sites are all of the methylation sites of Table 17.

144. The method of claim 115, wherein the one or more methylation sites are one or more methylation sites of Table 22.

145. The method of claim 115, wherein the one or more methylation sites are 2 or more methylation sites of Table 22.

146. The method of claim 115, wherein the one or more methylation sites are 3 or more methylation sites of Table 22.

147. The method of claim 115, wherein the one or more methylation sites are 4 or more methylation sites of Table 22.

148. The method of claim 115, wherein the one or more methylation sites are 5 or more methylation sites of Table 22.

149. The method of claim 115, wherein the one or more methylation sites are 10 or more methylation sites of Table 22.

150. The method of claim 115, wherein the one or more methylation sites are all of the methylation sites of Table 22.

151. The method of claim 115, wherein the one or more methylation sites are one or more methylation sites of Table 29.

152. The method of claim 115, wherein the one or more methylation sites are 2 or more methylation sites of Table 29.

153. The method of claim 115, wherein the one or more methylation sites are 3 or more methylation sites of Table 29.

154. The method of claim 115, wherein the one or more methylation sites are 4 or more methylation sites of Table 29.

155. The method of claim 115, wherein the one or more methylation sites are 5 or more methylation sites of Table 29.

156. The method of claim 115, wherein the one or more methylation sites are 10 or more methylation sites of Table 29.

157. The method of claim 115, wherein the one or more methylation sites are all of the methylation sites of Table 29.

158. The method of any of claims 115-157, wherein the tumor DNA is ctDNA.

159. The method of any of claims 115-136, wherein the method comprises administering to the subject the platinum-containing chemotherapeutic agent.

160. The method of claim 159, wherein the platinum-containing chemotherapeutic agent is cisplatin, carboplatin, oxaliplatin, nedaplatin, picoplatin, or satraplatin.

161. The method of any of claims 115-136, wherein the method comprises administering to the subject the PARP inhibitor.

162. The method of claim 161, wherein the PARP inhibitor is talazoparib, olaparib, niraparib, AZD-2461, or rucaparib.

163. The method of any of claims 115-136, wherein the method comprises administering to the subject the anti-metabolite.

164. The method of claim 163, wherein the anti-metabolite is pemetrexed, methotrexate, or pralatrexate.

165. The method of any of claims 115-136, wherein the method comprises administering to the subject the nucleoside analog.

166. The method of claim 165, wherein the nucleoside analog is floxuridine, cytarabine, clofarabine, or fludarabine.

167. The method of any of claims 115-166, further comprising administering to the subject an additional cancer therapy.

168. The method of claim 167, wherein the additional cancer therapy comprises chemotherapy, radiotherapy, immunotherapy, or a combination thereof.

169. The method of any of claims 115-168, wherein the subject was previously treated for SCLC.

170. The method of claim 169, wherein the subject was resistant to the previous treatment.

171. The method of any of claims 115-170, wherein the subject was determined to have SCLC-N based on the analysis of the tumor DNA.

172. The method of any of claims 115-171, wherein the subject was further determined, from the analysis of the tumor DNA from the subject, to have differential methylation of one or more methylation sites of Table 13 compared to the reference or control sample.

173. The method of any of claims 115-172, wherein the reference or control sample is a DNA sample obtained from healthy cells from the subject.

174. The method of any of claims 115-172, wherein the reference or control sample is a DNA sample obtained from healthy cells from a reference subject.

175. A method of treating a subject for SCLC, the method comprising administering an immunotherapy to a subject having tumor DNA with differential methylation at one or more methylation sites selected from the methylation sites of Table 5, Table 10, Table 18, Table 23, and Table 30 compared to a reference or control sample.

176. The method of claim 175, wherein the one or more methylation sites are 2 or more methylation sites selected from the methyation sites of Table 5, Table 10, Table 18, Table 23, and Table 30.

177. The method of claim 175, wherein the one or more methylation sites are 3 or more methylation sites selected from the methyation sites of Table 5, Table 10, Table 18, Table 23, and Table 30.

178. The method of claim 175, wherein the one or more methylation sites are 4 or more methylation sites selected from the methyation sites of Table 5, Table 10, Table 18, Table 23, and Table 30.

179. The method of claim 175, wherein the one or more methylation sites are 5 or more methylation sites selected from the methyation sites of Table 5, Table 10, Table 18, Table 23, and Table 30.

180. The method of claim 175, wherein the one or more methylation sites are 5 or more methylation sites selected from the methyation sites of Table 5, Table 10, Table 18, Table 23, and Table 30.

181. The method of claim 175, wherein the one or more methylation sites are 10 or more methylation sites selected from the methyation sites of Table 5, Table 10, Table 18, Table 23, and Table 30.

182. The method of claim 175, wherein the one or more methylation sites are twenty or more methylation sites selected from the methyation sites of Table 5, Table 10, Table 18, Table 23, and Table 30.

183. The method of claim 175, wherein the one or more methylation sites are fifty or more methylation sites selected from the methyation sites of Table 5, Table 10, Table 18, Table 23, and Table 30.

184. The method of claim 175, wherein the one or more methylation sites are one or more methylation sites of Table 5.

185. The method of claim 175, wherein the one or more methylation sites are 2 or more methylation sites of Table 5.

186. The method of claim 175, wherein the one or more methylation sites are 3 or more methylation sites of Table 5.

187. The method of claim 175, wherein the one or more methylation sites are 4 or more methylation sites of Table 5.

188. The method of claim 175, wherein the one or more methylation sites are 5 or more methylation sites of Table 5.

189. The method of claim 175, wherein the one or more methylation sites are 10 or more methylation sites of Table 5.

190. The method of claim 175, wherein the one or more methylation sites are all of the methylation sites of Table 5.

191. The method of claim 175, wherein the one or more methylation sites are one or more methylation sites of Table 10.

192. The method of claim 175, wherein the one or more methylation sites are 2 or more methylation sites of Table 10.

193. The method of claim 175, wherein the one or more methylation sites are 3 or more methylation sites of Table 10.

194. The method of claim 175, wherein the one or more methylation sites are 4 or more methylation sites of Table 10.

195. The method of claim 175, wherein the one or more methylation sites are 5 or more methylation sites of Table 10.

196. The method of claim 175, wherein the one or more methylation sites are 10 or more methylation sites of Table 10.

197. The method of claim 175, wherein the one or more methylation sites are all of the methylation sites of Table 10.

198. The method of claim 175, wherein the one or more methylation sites are one or more methylation sites of Table 18.

199. The method of claim 175, wherein the one or more methylation sites are 2 or more methylation sites of Table 18.

200. The method of claim 175, wherein the one or more methylation sites are 3 or more methylation sites of Table 18.

201. The method of claim 175, wherein the one or more methylation sites are 4 or more methylation sites of Table 18.

202. The method of claim 175, wherein the one or more methylation sites are 5 or more methylation sites of Table 18.

203. The method of claim 175, wherein the one or more methylation sites are 10 or more methylation sites of Table 18.

204. The method of claim 175, wherein the one or more methylation sites are all of the methylation sites of Table 18.

205. The method of claim 175, wherein the one or more methylation sites are one or more methylation sites of Table 23.

206. The method of claim 175, wherein the one or more methylation sites are 2 or more methylation sites of Table 23.

207. The method of claim 175, wherein the one or more methylation sites are 3 or more methylation sites of Table 23.

208. The method of claim 175, wherein the one or more methylation sites are 4 or more methylation sites of Table 23.

209. The method of claim 175, wherein the one or more methylation sites are 5 or more methylation sites of Table 23.

210. The method of claim 175, wherein the one or more methylation sites are 10 or more methylation sites of Table 23.

211. The method of claim 175, wherein the one or more methylation sites are all of the methylation sites of Table 23.

212. The method of claim 175, wherein the one or more methylation sites are one or more methylation sites of Table 30.

213. The method of claim 175, wherein the one or more methylation sites are 2 or more methylation sites of Table 30.

214. The method of claim 175, wherein the one or more methylation sites are 3 or more methylation sites of Table 30.

215. The method of claim 175, wherein the one or more methylation sites are 4 or more methylation sites of Table 30.

216. The method of claim 175, wherein the one or more methylation sites are 5 or more methylation sites of Table 30.

217. The method of claim 175, wherein the one or more methylation sites are 10 or more methylation sites of Table 30.

218. The method of claim 175, wherein the one or more methylation sites are all of the methylation sites of Table 30.

219. The method of any of claims 175-218, wherein the tumor DNA is ctDNA.

220. The method of any of claims 175-219, wherein the immunotherapy is an immune checkpoint inhibitor therapy.

221. The method of any of claims 175-220, further comprising administering to the subject an additional cancer therapy.

222. The method of claim 221, wherein the additional cancer therapy comprises chemotherapy, radiotherapy, immunotherapy, or a combination thereof.

223. The method of any of claims 175-222, wherein the subject was previously treated for SCLC.

224. The method of claim 223, wherein the subject was resistant to the previous treatment.

225. The method of any of claims 175-224, wherein the subject was determined to have SCLC-I based on the analysis of the tumor DNA.

226. The method of any of claims 175-225, wherein the subject was further determined, from the analysis of the tumor DNA from the subject, to have differential methylation of one or more methylation sites of Table 13 compared to the reference or control sample.

227. The method of any of claims 175-226, wherein the reference or control sample is a DNA sample obtained from healthy cells from the subject.

228. The method of any of claims 175-226, wherein the reference or control sample is a DNA sample obtained from healthy cells from a reference subject.

229. A method for classifying a subject having small cell lung cancer (SCLC), the method comprising:
(a) determining, from DNA from the subject, a methylation status of one or more methylation sites selected from the methylation sites of Tables 1-10, 15-18, 20-23, and 27-30; and
(b) classifying the subject as having SCLC-A, SCLC-N, SCLC-P, or SCLC-I based on the methylation status of the one or more methylation sites.

230. The method of claim 229, wherein (b) comprises classifying the subject as having SCLC-A.

231. The method of claim 230, wherein the one or more methylation sites are one or more methylation sites selected from the methylation sites of Table 2, Table 7, Table 15, Table 20, and Table 27.

232. The method of claim 230, wherein the one or more methylation sites are 2 or more methylation sites selected from the methylation sites of Table 2, Table 7, Table 15, Table 20, and Table 27.

233. The method of claim 230, wherein the one or more methylation sites are 3 or more methylation sites selected from the methylation sites of Table 2, Table 7, Table 15, Table 20, and Table 27.

234. The method of claim 230, wherein the one or more methylation sites are 4 or more methylation sites selected from the methylation sites of Table 2, Table 7, Table 15, Table 20, and Table 27.

235. The method of claim 232, wherein the one or more methylation sites are 5 or more methylation sites selected from the methyation sites of Table 2, Table 7, Table 15, Table 20, and Table 27.

236. The method of claim 232, wherein the one or more methylation sites are 10 or more methylation sites selected from the methyation sites of Table 2, Table 7, Table 15, Table 20, and Table 27.

237. The method of claim 232, wherein the one or more methylation sites are twenty or more methylation sites selected from the methyation sites of Table 2, Table 7, Table 15, Table 20, and Table 27.

238. The method of claim 232, wherein the one or more methylation sites are fifty or more methylation sites selected from the methyation sites of Table 2, Table 7, Table 15, Table 20, and Table 27.

239. The method of claim 230, wherein the one or more methylation sites are one or more methylation sites of Table 2.

240. The method of claim 230, wherein the one or more methylation sites are 2 or more methylation sites of Table 2.

241. The method of claim 230, wherein the one or more methylation sites are 3 or more methylation sites of Table 2.

242. The method of claim 230, wherein the one or more methylation sites are 4 or more methylation sites of Table 2.

243. The method of claim 230, wherein the one or more methylation sites are 5 or more methylation sites of Table 2.

244. The method of claim 230, wherein the one or more methylation sites are 10 or more methylation sites of Table 2.

245. The method of claim 230, wherein the one or more methylation sites are all of the methylation sites of Table 2.

246. The method of claim 230, wherein the one or more methylation sites are one or more methylation sites of Table 7.

247. The method of claim 230, wherein the one or more methylation sites are 2 or more methylation sites of Table 7.

248. The method of claim 230, wherein the one or more methylation sites are 3 or more methylation sites of Table 7.

249. The method of claim 230, wherein the one or more methylation sites are 4 or more methylation sites of Table 7.

250. The method of claim 230, wherein the one or more methylation sites are 5 or more methylation sites of Table 7.

251. The method of claim 230, wherein the one or more methylation sites are 10 or more methylation sites of Table 7.

252. The method of claim 230, wherein the one or more methylation sites are all of the methylation sites of Table 7.

253. The method of claim 230, wherein the one or more methylation sites are one or more methylation sites of Table 15.

254. The method of claim 230, wherein the one or more methylation sites are 2 or more methylation sites of Table 15.

255. The method of claim 230, wherein the one or more methylation sites are 3 or more methylation sites of Table 15.

256. The method of claim 230, wherein the one or more methylation sites are 4 or more methylation sites of Table 15.

257. The method of claim 230, wherein the one or more methylation sites are 5 or more methylation sites of Table 15.

258. The method of claim 230, wherein the one or more methylation sites are 10 or more methylation sites of Table 15.

259. The method of claim 230, wherein the one or more methylation sites are all of the methylation sites of Table 15.

260. The method of claim 230, wherein the one or more methylation sites are one or more methylation sites of Table 20.

261. The method of claim 230, wherein the one or more methylation sites are 2 or more methylation sites of Table 20.

262. The method of claim 230, wherein the one or more methylation sites are 3 or more methylation sites of Table 20.

263. The method of claim 230, wherein the one or more methylation sites are 4 or more methylation sites of Table 20.

264. The method of claim 230, wherein the one or more methylation sites are 5 or more methylation sites of Table 20.

265. The method of claim 230, wherein the one or more methylation sites are 10 or more methylation sites of Table 20.

266. The method of claim 230, wherein the one or more methylation sites are all of the methylation sites of Table 20.

267. The method of claim 230, wherein the one or more methylation sites are one or more methylation sites of Table 27.

268. The method of claim 230, wherein the one or more methylation sites are 2 or more methylation sites of Table 27.

269. The method of claim 230, wherein the one or more methylation sites are 3 or more methylation sites of Table 27.

270. The method of claim 230, wherein the one or more methylation sites are 4 or more methylation sites of Table 27.

271. The method of claim 230, wherein the one or more methylation sites are 50 or more methylation sites of Table 27.

272. The method of claim 230, wherein the one or more methylation sites are 10 or more methylation sites of Table 27.

273. The method of claim 230, wherein the one or more methylation sites are all of the methylation sites of Table 27.

274. The method of any of claims 230-273, further comprising administering to the subject a therapeutically effective amount of a BCL2 inhibitor.

275. The method of claim 274, wherein the BCL2 inhibitor is ABT-737 or navitoclax.

276. The method of any of claims 230-274, further comprising administering to the subject a therapeutically effective amount of a DLL3-targeted therapeutic.

277. The method of claim 276, wherein the DLL3-targeted therapeutic comprises an anti-DLL3 antibody or fragment thereof.

278. The method of claim 277, wherein the anti-DLL3 antibody or fragment thereof is rovalpituzumab.

279. The method of claim 276, wherein the DLL3-targeted therapeutic is an antibody-drug conjugate.

280. The method of claim 279, wherein the antibody-drug conjugate is rovalpituzumab tesirine.

281. The method of claim 229, wherein (b) comprises classifying the subject as having SCLC-N.

282. The method of claim 281, wherein the one or more methylation sites are one or more methylation sites selected from the methyation sites of Table 3, Table 8, Table 16, Table 21, and Table 28.

283. The method of claim 281, wherein the one or more methylation sites are 2 or more methylation sites selected from the methyation sites of Table 3, Table 8, Table 16, Table 21, and Table 28.

284. The method of claim 281, wherein the one or more methylation sites are 3 or more methylation sites selected from the methyation sites of Table 3, Table 8, Table 16, Table 21, and Table 28.

285. The method of claim 281, wherein the one or more methylation sites are 4 or more methylation sites selected from the methyation sites of Table 3, Table 8, Table 16, Table 21, and Table 28.

286. The method of claim 281, wherein the one or more methylation sites are 5 or more methylation sites selected from the methyation sites of Table 3, Table 8, Table 16, Table 21, and Table 28.

287. The method of claim 281, wherein the one or more methylation sites are 10 or more methylation sites selected from the methyation sites of Table 3, Table 8, Table 16, Table 21, and Table 28.

288. The method of claim 281, wherein the one or more methylation sites are twenty or more methylation sites selected from the methyation sites of Table 3, Table 8, Table 16, Table 21, and Table 28.

289. The method of claim 281, wherein the one or more methylation sites are fifty or more methylation sites selected from the methyation sites of Table 3, Table 8, Table 16, Table 21, and Table 28.

290. The method of claim 281, wherein the one or more methylation sites are one or more methylation sites of Table 3.

291. The method of claim 281, wherein the one or more methylation sites are 2 or more methylation sites of Table 3.

292. The method of claim 281, wherein the one or more methylation sites are 3 or more methylation sites of Table 3.

293. The method of claim 281, wherein the one or more methylation sites are 4 or more methylation sites of Table 3.

294. The method of claim 281, wherein the one or more methylation sites are 5 or more methylation sites of Table 3.

295. The method of claim 281, wherein the one or more methylation sites are 10 or more methylation sites of Table 3.

296. The method of claim 281, wherein the one or more methylation sites are all of the methylation sites of Table 3.

297. The method of claim 281, wherein the one or more methylation sites are one or more methylation sites of Table 8.

298. The method of claim 281, wherein the one or more methylation sites are 2 or more methylation sites of Table 8.

299. The method of claim 281, wherein the one or more methylation sites are 3 or more methylation sites of Table 8.

300. The method of claim 281, wherein the one or more methylation sites are 4 or more methylation sites of Table 8.

301. The method of claim 281, wherein the one or more methylation sites are 5 or more methylation sites of Table 8.

302. The method of claim 281, wherein the one or more methylation sites are 10 or more methylation sites of Table 8.

303. The method of claim 281, wherein the one or more methylation sites are all of the methylation sites of Table 8.

304. The method of claim 281, wherein the one or more methylation sites are one or more methylation sites of Table 16.

305. The method of claim 281, wherein the one or more methylation sites are 2 or more methylation sites of Table 16.

306. The method of claim 281, wherein the one or more methylation sites are 3 or more methylation sites of Table 16.

307. The method of claim 281, wherein the one or more methylation sites are 4 or more methylation sites of Table 16.

308. The method of claim 281, wherein the one or more methylation sites are 5 or more methylation sites of Table 16.

309. The method of claim 281, wherein the one or more methylation sites are 10 or more methylation sites of Table 16.

310. The method of claim 281, wherein the one or more methylation sites are all of the methylation sites of Table 16.

311. The method of claim 281, wherein the one or more methylation sites are one or more methylation sites of Table 21.

312. The method of claim 281, wherein the one or more methylation sites are 2 or more methylation sites of Table 21.

313. The method of claim 281, wherein the one or more methylation sites are 3 or more methylation sites of Table 21.

314. The method of claim 281, wherein the one or more methylation sites are 4 or more methylation sites of Table 21.

315. The method of claim 281, wherein the one or more methylation sites are 5 or more methylation sites of Table 21.

316. The method of claim 281, wherein the one or more methylation sites are 10 or more methylation sites of Table 21.

317. The method of claim 281, wherein the one or more methylation sites are all of the methylation sites of Table 21.

318. The method of claim 281, wherein the one or more methylation sites are one or more methylation sites of Table 28.

319. The method of claim 281, wherein the one or more methylation sites are 2 or more methylation sites of Table 28.

320. The method of claim 281, wherein the one or more methylation sites are 3 or more methylation sites of Table 28.

321. The method of claim 281, wherein the one or more methylation sites are 4 or more methylation sites of Table 28.

322. The method of claim 281, wherein the one or more methylation sites are 5 or more methylation sites of Table 28.

323. The method of claim 281, wherein the one or more methylation sites are 10 or more methylation sites of Table 28.

324. The method of claim 281, wherein the one or more methylation sites are all of the methylation sites of Table 28.

325. The method of claim 281-324, further comprising administering to the subject a therapeutically effective amount of an AURK inhibitor.

326. The method of claim 325, wherein the AURK inhibitor is CYC-116, alisertib, or AS-703569.

327. The method of claim 229, wherein (b) comprises classifying the subject as having SCLC-P.

328. The method of claim 327, wherein the one or more methylation sites are one or more methylation sites selected from the methyation sites of Table 4, Table 9, Table 17, Table 22, and Table 29.

329. The method of claim 327, wherein the one or more methylation sites are 2 or more methylation sites selected from the methyation sites of Table 4, Table 9, Table 17, Table 22, and Table 29.

330. The method of claim 327, wherein the one or more methylation sites are 3 or more methylation sites selected from the methyation sites of Table 4, Table 9, Table 17, Table 22, and Table 29.

331. The method of claim 327, wherein the one or more methylation sites are 4 or more methylation sites selected from the methyation sites of Table 4, Table 9, Table 17, Table 22, and Table 29.

332. The method of claim 327, wherein the one or more methylation sites are 5 or more methylation sites selected from the methyation sites of Table 4, Table 9, Table 17, Table 22, and Table 29.

333. The method of claim 327, wherein the one or more methylation sites are 10 or more methylation sites selected from the methyation sites of Table 4, Table 9, Table 17, Table 22, and Table 29.

334. The method of claim 327, wherein the one or more methylation sites are twenty or more methylation sites selected from the methyation sites of Table 4, Table 9, Table 17, Table 22, and Table 29.

335. The method of claim 327, wherein the one or more methylation sites are fifty or more methylation sites selected from the methyation sites of Table 4, Table 9, Table 17, Table 22, and Table 29.

336. The method of claim 327, wherein the one or more methylation sites are one or more methylation sites of Table 4.

337. The method of claim 327, wherein the one or more methylation sites are 2 or more methylation sites of Table 4.

338. The method of claim 327, wherein the one or more methylation sites are three or more methylation sites of Table 4.

339. The method of claim 327, wherein the one or more methylation sites are 4 or more methylation sites of Table 4.

340. The method of claim 327, wherein the one or more methylation sites are 5 or more methylation sites of Table 4.

341. The method of claim 327, wherein the one or more methylation sites are 10 or more methylation sites of Table 4.

342. The method of claim 327, wherein the one or more methylation sites are all of the methylation sites of Table 4.

343. The method of claim 327, wherein the one or more methylation sites are one or more methylation sites of Table 9.

344. The method of claim 327, wherein the one or more methylation sites are 2 or more methylation sites of Table 9.

345. The method of claim 327, wherein the one or more methylation sites are 3 or more methylation sites of Table 9.

346. The method of claim 327, wherein the one or more methylation sites are 4 or more methylation sites of Table 9.

347. The method of claim 327, wherein the one or more methylation sites are 5 or more methylation sites of Table 9.

348. The method of claim 327, wherein the one or more methylation sites are 10 or more methylation sites of Table 9.

349. The method of claim 327, wherein the one or more methylation sites are all of the methylation sites of Table 9.

350. The method of claim 327, wherein the one or more methylation sites are one or more methylation sites of Table 17.

351. The method of claim 327, wherein the one or more methylation sites are 2 or more methylation sites of Table 17.

352. The method of claim 327, wherein the one or more methylation sites are 3 or more methylation sites of Table 17.

353. The method of claim 327, wherein the one or more methylation sites are 4 or more methylation sites of Table 17.

354. The method of claim 327, wherein the one or more methylation sites are 5 or more methylation sites of Table 17.

355. The method of claim 327, wherein the one or more methylation sites are 10 or more methylation sites of Table 17.

356. The method of claim 327, wherein the one or more methylation sites are all of the methylation sites of Table 17.

357. The method of claim 327, wherein the one or more methylation sites are one or more methylation sites of Table 22.

358. The method of claim 327, wherein the one or more methylation sites are 2 or more methylation sites of Table 22.

359. The method of claim 327, wherein the one or more methylation sites are 3 or more methylation sites of Table 22.

360. The method of claim 327, wherein the one or more methylation sites are 4 or more methylation sites of Table 22.

361. The method of claim 327, wherein the one or more methylation sites are 5 or more methylation sites of Table 22.

362. The method of claim 327, wherein the one or more methylation sites are 10 or more methylation sites of Table 22.

363. The method of claim 327, wherein the one or more methylation sites are all of the methylation sites of Table 22.

364. The method of claim 327, wherein the one or more methylation sites are one or more methylation sites of Table 29.

365. The method of claim 327, wherein the one or more methylation sites are 2 or more methylation sites of Table 29.

366. The method of claim 327, wherein the one or more methylation sites are 3 or more methylation sites of Table 29.

367. The method of claim 327, wherein the one or more methylation sites are 4 or more methylation sites of Table 29.

368. The method of claim 327, wherein the one or more methylation sites are 5 or more methylation sites of Table 29.

369. The method of claim 327, wherein the one or more methylation sites are 10 or more methylation sites of Table 29.

370. The method of claim 327, wherein the one or more methylation sites are all of the methylation sites of Table 29.

371. The method of any of claims 327-370, further comprising administering to the subject a therapeutically effective amount of a platinum-containing chemotherapeutic agent.

372. The method of claim 371, wherein the platinum-containing chemotherapeutic agent is cisplatin, carboplatin, oxaliplatin, nedaplatin, picoplatin, or satraplatin.

373. The method of any of claims 327-349, further comprising administering to the subject a therapeutically effective amount of a PARP inhibitor.

374. The method of claim 373, wherein the PARP inhibitor is talazoparib, olaparib, niraparib, AZD-2461, or rucaparib.

375. The method of any of claims 327-349, further comprising administering to the subject a therapeutically effective amount of an anti-metabolite.

376. The method of claim 375, wherein the anti-metabolite is pemetrexed, methotrexate, or pralatrexate.

377. The method of any of claims 327-349, further comprising administering to the subject a therapeutically effective amount of a nucleoside analog.

378. The method of claim 377, wherein the nucleoside analog is floxuridine, cytarabine, clofarabine, or fludarabine.

379. The method of claim 229, wherein (b) comprises classifying the subject as having SCLC-I.

380. The method of claim 379, wherein the one or more methylation sites are one or more methylation sites selected from the methyation sites of Table 5, Table 10, Table 18, Table 23, and Table 30.

381. The method of claim 379, wherein the one or more methylation sites are 2 or more methylation sites selected from the methyation sites of Table 5, Table 10, Table 18, Table 23, and Table 30.

382. The method of claim 379, wherein the one or more methylation sites are 3 or more methylation sites selected from the methyation sites of Table 5, Table 10, Table 18, Table 23, and Table 30.

383. The method of claim 379, wherein the one or more methylation sites are 4 or more methylation sites selected from the methyation sites of Table 5, Table 10, Table 18, Table 23, and Table 30.

384. The method of claim 379, wherein the one or more methylation sites are 5 or more methylation sites selected from the methyation sites of Table 5, Table 10, Table 18, Table 23, and Table 30.

385. The method of claim 379, wherein the one or more methylation sites are 10 or more methylation sites selected from the methyation sites of Table 5, Table 10, Table 18, Table 23, and Table 30.

386. The method of claim 379, wherein the one or more methylation sites are twenty or more methylation sites selected from the methyation sites of Table 5, Table 10, Table 18, Table 23, and Table 30.

387. The method of claim 379, wherein the one or more methylation sites are fifty or more methylation sites selected from the methyation sites of Table 5, Table 10, Table 18, Table 23, and Table 30.

388. The method of claim 379, wherein the one or more methylation sites are one or more methylation sites of Table 5.

389. The method of claim 379, wherein the one or more methylation sites are 2 or more methylation sites of Table 5.

390. The method of claim 379, wherein the one or more methylation sites are 3 or more methylation sites of Table 5.

391. The method of claim 379, wherein the one or more methylation sites are 4 or more methylation sites of Table 5.

392. The method of claim 379, wherein the one or more methylation sites are 5 or more methylation sites of Table 5.

393. The method of claim 379, wherein the one or more methylation sites are 10 or more methylation sites of Table 5.

394. The method of claim 379, wherein the one or more methylation sites are all of the methylation sites of Table 5.

395. The method of claim 379, wherein the one or more methylation sites are one or more methylation sites of Table 10.

396. The method of claim 379, wherein the one or more methylation sites are 2 or more methylation sites of Table 10.

397. The method of claim 379, wherein the one or more methylation sites are 3 or more methylation sites of Table 10.

398. The method of claim 379, wherein the one or more methylation sites are 4 or more methylation sites of Table 10.

399. The method of claim 379, wherein the one or more methylation sites are 5 or more methylation sites of Table 10.

400. The method of claim 379, wherein the one or more methylation sites are 10 or more methylation sites of Table 10.

401. The method of claim 379, wherein the one or more methylation sites are all of the methylation sites of Table 10.

402. The method of claim 379, wherein the one or more methylation sites are one or more methylation sites of Table 18.

403. The method of claim 379, wherein the one or more methylation sites are 2 or more methylation sites of Table 18.

404. The method of claim 379, wherein the one or more methylation sites are 3 or more methylation sites of Table 18.

405. The method of claim 379, wherein the one or more methylation sites are 4 or more methylation sites of Table 18.

406. The method of claim 379, wherein the one or more methylation sites are 5 or more methylation sites of Table 18.

407. The method of claim 379, wherein the one or more methylation sites are 10 or more methylation sites of Table 18.

408. The method of claim 379, wherein the one or more methylation sites are all of the methylation sites of Table 18.

409. The method of claim 379, wherein the one or more methylation sites are one or more methylation sites of Table 23.

410. The method of claim 379, wherein the one or more methylation sites are 2 or more methylation sites of Table 23.

411. The method of claim 379, wherein the one or more methylation sites are 3 or more methylation sites of Table 23.

412. The method of claim 379, wherein the one or more methylation sites are 4 or more methylation sites of Table 23.

413. The method of claim 379, wherein the one or more methylation sites are 5 or more methylation sites of Table 23.

414. The method of claim 379, wherein the one or more methylation sites are 10 or more methylation sites of Table 23.

415. The method of claim 379, wherein the one or more methylation sites are all of the methylation sites of Table 23.

416. The method of claim 379, wherein the one or more methylation sites are one or more methylation sites of Table 30.

417. The method of claim 379, wherein the one or more methylation sites are 2 or more methylation sites of Table 30.

418. The method of claim 379, wherein the one or more methylation sites are 3 or more methylation sites of Table 30.

419. The method of claim 379, wherein the one or more methylation sites are 4 or more methylation sites of Table 30.

420. The method of claim 379, wherein the one or more methylation sites are 5 or more methylation sites of Table 30.

421. The method of claim 379, wherein the one or more methylation sites are 10 or more methylation sites of Table 30.

422. The method of claim 379, wherein the one or more methylation sites are all of the methylation sites of Table 30.

423. The method of any of claims 379-422, further comprising administering to the subject a therapeutically effective amount of an immunotherapy.

424. The method of claim 423, wherein the immunotherapy is a checkpoint blockade therapy.

425. The method of any of claims 379-401, further comprising administering to the subject a therapeutically effective amount of a BTK inhibitor.

426. The method of claim 425, wherein the BTK inhibitor is ibrutinib.

427. The method of any of claims 229-426, wherein the DNA is obtained from blood or plasma from the subject.

428. The method of any of claims 229-427, wherein the DNA is circulating tumor DNA (ctDNA).

429. The method of any of claims 229-426, wherein the DNA is obtained from cancer tissue from the subject.

430. The method of any of claims 229-429, further comprising determining, from the DNA from the subject, a methylation status of one or more methylation sites of Table 13.

431. The method of claim 430, wherein the one or more methylation sites are one or more methylation sites of Table 13.

432. The method of claim 430, wherein the one or more methylation sites are 5 or more methylation sites of Table 13.

433. A method of identifying a subject with cancer as having SCLC, the method comprising:
(a) determining, from DNA from the subject, a methylation status of one or more methylation sites of Table 13; and
(b) identifying the subject as having SCLC based on the methylation status of the one or more methylation sites.

434. The method of claim 433, wherein the one or more methylation sites are 2 or more methylation sites of Table 13.

435. The method of claim 433, wherein the one or more methylation sites are 5 or more methylation sites of Table 13.

436. The method of claim 433, wherein the one or more methylation sites are 10 or more methylation sites of Table 13.

437. The method of claim 433, wherein the one or more methylation sites are twenty or more methylation sites of Table 13.

438. The method of claim 433, wherein the one or more methylation sites are all of the methylation sites of Table 13.

439. The method of any of claims 433-438, wherein the DNA is obtained from blood or plasma from the subject.

440. The method of any of claims 433-439, wherein the DNA is ctDNA.

441. The method of any of claims 433-438, wherein the DNA is obtained from cancer tissue from the subject.

442. The method of any of claims 433-441, further comprising treating the subject for SCLC.

443. A method for treating a subject for SCLC comprising administering an SCLC therapy to a subject determined, from analysis of tumor DNA from the subject, to have differential methylation at one or more methylation sites of Table 13 compared to a reference or control sample.

444. The method of claim 443, wherein the SCLC therapy comprises chemotherapy, immunotherapy, radiotherapy, or a combination thereof.

445. The method of claim 443 or 444, wherein the SCLC therapy comprises a platinum-containing chemotherapeutic agent.

446. The method of claim 445, wherein the platinum-containing chemotherapeutic agent is cisplatin, carboplatin, oxaliplatin, nedaplatin, picoplatin, or satraplatin.

447. The method of any of claims 443-446, wherein the one or more methylation sites are 2 or more methylation sites of Table 13.

448. The method of any of claims 443-446, wherein the one or more methylation sites are 5 or more methylation sites of Table 13.

449. A method of evaluating tumor burden in a subject having SCLC, the method comprising determining, from DNA from the subject, a methylation status of one or more methylation sites of Table 24.

450. The method of claim 449, wherein the one or more methylation sites are 2 or more methylation sites of Table 24.

451. The method of claim 449, wherein the one or more methylation sites are 3 or more methylation sites of Table 24.

452. The method of claim 449, wherein the one or more methylation sites are 4 or more methylation sites of Table 24.

453. The method of claim 449, wherein the one or more methylation sites are 5 or more methylation sites of Table 24.

454. The method of claim 449, wherein the one or more methylation sites are 10 or more methylation sites of Table 24.

455. The method of claim 449, wherein the one or more methylation sites are twenty or more methylation sites of Table 24.

456. The method of claim 449, wherein the one or more methylation sites are all of the methylation sites of Table 24.

457. The method of any of claims 449-456, wherein the DNA is obtained from blood or plasma from the subject.

458. The method of any of claims 449-456, wherein the DNA is ctDNA.

459. The method of any of claims 449-456, wherein the DNA is obtained from cancer tissue from the subject.

460. A method for determining a response of a subject having SCLC to a cancer therapy, the method comprising determining, from DNA from the subject, a methylation status of one or more methylation sites of Table 24.

461. The method of claim 460, wherein the one or more methylation sites are 2 or more methylation sites of Table 24.

462. The method of claim 460, wherein the one or more methylation sites are 3 or more methylation sites of Table 24.

463. The method of claim 460, wherein the one or more methylation sites are 5 or more methylation sites of Table 24.

464. The method of claim 460, wherein the one or more methylation sites are 10 or more methylation sites of Table 24.

465. The method of claim 460, wherein the one or more methylation sites are twenty or more methylation sites of Table 24.

466. The method of claim 460, wherein the one or more methylation sites are all of the methylation sites of Table 24.

467. The method of any of claims 460-466, wherein the DNA is obtained from blood or plasma from the subject.

468. The method of any of claims 460-466, wherein the DNA is ctDNA.

469. The method of any of claims 460-466, wherein the DNA is obtained from cancer tissue from the subject.

470. A method for treating a subject having SCLC, the method comprising:
(a) determining, from DNA from the subject, a methylation level of one or more methylation sites of Table 24;
(b) administering a dose of a first cancer therapy to the subject;
(c) determining, from DNA from the subject, an additional methylation level of the one or more methylation sites of Table 24; and
if the additional methylation level is not increased compared with the methylation level, administering an additional dose of the first cancer therapy to the subject: or
if the additional methylation level is increased compared with the methylation level, administering to the subject a second cancer therapy that is different from the first cancer therapy.

471. The method of claim 470, wherein the one or more methylation sites are 2 or more methylation sites of Table 24.

472. The method of claim 470, wherein the one or more methylation sites are 3 or more methylation sites of Table 24.

473. The method of claim 470, wherein the one or more methylation sites are 5 or more methylation sites of Table 24.

474. The method of claim 470, wherein the one or more methylation sites are 10 or more methylation sites of Table 24.

475. The method of claim 470, wherein the one or more methylation sites are twenty or more methylation sites of Table 24.

476. The method of claim 470, wherein the one or more methylation sites are all of the methylation sites of Table 24.

477. The method of any of claims 470-476, wherein the DNA is obtained from blood or plasma from the subject.

478. The method of any of claims 470-476, wherein the DNA is ctDNA.

479. The method of any of claims 470-476, wherein the DNA is obtained from cancer tissue from the subject.

480. The method of any of claims 470-479, wherein the method comprises administering an additional dose of the cancer therapy to the subject if the additional methylation level is reduced compared with the methylation level.

481. The method of claim 480, wherein the method comprises administering the additional dose of the cancer therapy to the subject if the additional methylation level is reduced by at least 10% compared with the methylation level.

482. The method of claim 481, wherein the method comprises administering the additional dose of the cancer therapy to the subject if the additional methylation level is reduced by at least 20% compared with the methylation level.
